(19) United States
(12) Patent Application Publication
(10) Pub. No.: US 2014/0065172 A1
(43) Pub. Date: Mar. 6, 2014

(54) DELIVERY SYSTEM AND CONJUGATES FOR COMPOUND DELIVERY VIA NATURALLY OCCURRING INTRACELLULAR TRANSPORT ROUTES

(71) Applicant: CENIX BIOSCIENCE GMBH, Dresden (DE)

(72) Inventors: Christophe J. Echeverri, Roseville, MN (US); Birte Sönnichsen, Dresden (DE); Reinhard Wähler, Wenden (DE); Mike Werner Helms, Kriftel (DE); Brian S. Sproat, Booischot (BE)

(21) Appl. No.: 13/982,105

(22) PCT Filed: Jan. 26, 2012
(86) PCT No.: PCT/EP2012/051274

§ 371 Date: Nov. 15, 2013

Related U.S. Application Data
(60) Provisional application No. 61/436,579, filed on Jan. 26, 2011.

(30) Foreign Application Priority Data
Jan. 27, 2011 (EP) 11000673.1

(51) Int. Cl. C12N 15/113

(52) U.S. Cl. CPC C12N 15/113
USPC 424/179.1; 530/358; 514/21.2; 435/375; 435/256.1; 435/255.1; 435/255.2; 435/255.5; 435/348; 435/358; 435/363; 435/366; 435/410

(57) ABSTRACT

The present invention relates to a delivery system that comprises a conjugate that facilitates the delivery of a compound such as a biologically-active macromolecule, a nucleic acid or a peptide in particular, into a cell. The present invention also relates to said conjugate for delivery of a compound, such as a biologically-active macromolecule, a nucleic acid or a peptide, into a cell. The present invention further relates to a pharmaceutical composition comprising said conjugate and to its use. The present invention also relates to a method of delivering a compound to a cell or an organism, preferably a patient.

### CROSS REFERENCES

This application is a US national phase entry for PCT/EP2012/051274, filed on Jan. 26, 2012, which claims the priority date of U.S. 61/436,579, filed on Jan. 26, 2011, and EP 11000673.1, filed on Jan. 27, 2011. The disclosure of therein is incorporated entirely.

### FIELD OF INVENTION

The present invention relates to a delivery system that comprises a conjugate that facilitates the delivery of a compound such as a biologically-active macromolecule, a nucleic acid or a peptide in particular, into a cell. The present invention also relates to said conjugate for delivery of a compound, such as a biologically-active macromolecule, nucleic acid or peptide, into a cell. The present invention further relates to a pharmaceutical composition comprising said conjugate and to its use. The present invention also relates to a method of delivering a compound to a cell or organism, such as a patient.

### BACKGROUND OF THE INVENTION

New therapies are under development, which seek to address diseased states at the molecular level. A major problem in the practical application of many of these new therapeutic compounds is that the compounds do not readily cross cellular membranes and, thus, cannot reach compartments within the cell where their sites of action may reside.

The inability of most large molecules to efficiently cross the plasma membrane of animal cells has typically restricted their application for research and therapeutic purposes to those involving mechanisms of action occurring outside of the cells, most often through interactions on the cell surface. However, certain types of biologically-active macromolecules, such as antisense oligonucleotides, ribozymes, RNAi-inducing nucleic acid duplexes such as siRNAs and longer nucleic acids such as plasmids, must be present within intracellular compartments such as the cytosol or the nucleus to produce their intended biological effects. Unfortunately, in addition to the problem posed by the high net charges typically carried by such molecules for getting across the hydrophobic environment of cellular membranes, their overall size also greatly exceeds the upper limits, generally estimated at around 500 Da, of what can readily diffuse across those membranes unassisted. As such, the utility of these molecules for both research and therapeutic applications is strongly dependent on the use of delivery technologies designed to facilitate their efficient accumulation at their intended site of activity.

While in vitro applications in cultured cells require this delivery process to also include the transfer of the macromolecules intact through the growth medium, in vivo applications in living animals often impose a more challenging path. This starts with introduction into the body, continues with passage through various body fluids, tissues and structures, any of which may present significant chemical or physical barriers, and ends with eventual entry into the targeted cells to reach the intended site of action. For the in vivo context, this process also implies the need to avoid or at least delay excretion out of the body long enough to allow useful amounts of uptake into targeted cells. In all contexts, the delivery solution must also minimize undesired modifications either to the introduced molecules, or to any of the tissues, fluids, structures and cells encountered along the way. For example, many lipid-based nanoparticles and liposomal formulations are significantly limited in their applicability by their restricted bio-distribution (accumulating primarily in the liver) and their inherent risks for causing cytotoxic effects [1].

In some cases, minimizing risks of undesirable secondary effects can also imply preventing unwanted interactions of the delivered macromolecules with unintended binding partners along the way. Examples of this include unspecific immune stimulation that can be unintentionally triggered by certain nucleic acid constructs. While some delivery technologies help to resolve this problem by physically shielding or encapsulating the macromolecule during transit and only releasing it or activating it at the appropriate time/location (see, for example, WO 2009/045457), others lack this functionality and rely on optimization of the molecule itself to address this issue. In the case of siRNAs and other RNAi-inducing agents, the latter has indeed been possible, both by avoiding sequence motifs known to bear higher risks of immune stimulation, and through chemical alterations to the nucleic acid backbone, which render such molecules poor substrates for unintended pathways [such as Toll Like Receptor (TLR)-based immune responses], while preserving maximal activity with the targeted machinery [such as the RNA-induced Silencing Complex (RISC)].

Ultimately, once the delivery vehicle has successfully brought its cargo to the surface of the targeted cells, it still faces one of the most formidable barriers common to all delivery paths, i.e. the targeted cell's plasma membrane, through which, as noted above, large and/or highly charged macromolecules typically cannot pass unassisted. While some delivery technologies attempt to address this by triggering cellular uptake through natural internalization processes such as endocytosis, pinocytosis or phagocytosis, all such currently-available solutions only delay the problem without actually solving it, since access to the cytosol will still require the same membrane to be crossed from within the resulting endocytic, pinocytic or phagocytic vesicles. Indeed, the successful crossing of this crucial biological membrane, whether it occurs on the cell surface or from within such intracellular vesicles, has proven to be a particularly challenging and rate-limiting step for virtually all delivery technologies tested to date.

One common approach to addressing this challenge has been to take advantage of the acidification process that virtually all cells naturally drive inside many newly-internalized vesicles of endocytic, pinocytic or phagocytic origin, typically as these get sorted towards a lysosomal fate. To this end, these delivery technologies integrate various molecules, which carry a pH-dependent ability to “force” the destabilization or permeabilization of these vesicular membranes under appropriately acidic conditions, and hopefully before the delivered molecules get damaged in the lysosome. Sometimes referred to as “endosomolytic activity”, this form of endosomal escape has been realized through several different strategies in recent years [discussed in US 2008/0200661 A1, including the inclusion of fusogenic lipids within liposomes and so-called stable nucleic acid lipid particles (SNALPs)]. Another example makes use of so-called peptide transduction domains (PTDs) derived from various proteins that have naturally evolved to mediate the transfer of macromolecules or even larger cargo such as entire viruses across cellular membranes, including some known to become activated by acidication of the endosome (US 2006/0222657 A1). A third notable example has been the use of PBAVE, an amphipathic poly(vinyl ether) whose endosomolytic activity was reversibly shielded by PEG groups linked via acid-labile maleamate bonds [2, and US 2007/0036865 A1). However, despite the variable successes noted with such technologies to date, their “forced endosomal escape” processes still represent the key rate-limiting step in most, if not all, of these solutions, thus indicating that these approaches have still not met this challenge optimally.

Finally, an important but often-overlooked issue in designing delivery solutions is the question of what happens to the delivery vehicle or construct once it has completed its mission. The possibility that these delivery molecules will fail to be metabolized and will thus accumulate within the targeted cells imposes a further requirement on the design of these molecules, especially in the context of repeated or sustained long-term treatments. In particular, the components used within the delivery vehicles or constructs should not cause any deleterious effects in this context. As a result, delivery molecules that are known to be readily and safely metabolized by targeted cells present a preferred solution, whereas those making use of artificial, non-biodegradable chemistries or molecules whose long-term effects have not been adequately characterized present increased risks.

Thus, there is an urgent need for a delivery system that can efficiently deliver compounds such as biologically-active macromolecules, nucleic acids or peptides in particular, into living cells. There is also an urgent need for a delivery system that does not cause any deleterious side effects within the cell. A delivery system that utilizes components that are readily and safely metabolized by targeted cells would also be highly desirable.

### SUMMARY OF THE INVENTION

The present invention relates to a delivery system that comprises a conjugate that facilitates the delivery of a compound such as a biologically-active macromolecule, a nucleic acid or a peptide in particular, into living cells of interest, preferably into the cytosol or nucleus of said living cells of interest. The delivery systems and conjugates of the present invention are designed to harness and/or exploit fully natural pathways for initial cell targeting and internalization, followed by retrograde transport through membranous compartments to the endoplasmic reticulum (ER) and retro-translocation from the ER to the cytosol via the ER-associated degradation pathway (ERAD). Upon reaching the cytosol, the delivery systems and conjugates of the present invention may either deliver a compound to the cytosol or continue on to deliver a compound to the nucleus.

As such, the present invention provides delivery systems and conjugates which can effectively deliver compounds such as biologically active macromolecules, nucleic acids or peptides in particular, to a targeted cytosol or nucleus by using endogenous processes that occur ubiquitously within all cells. The conjugates of the present invention maximally utilize and exploit the benefits of these endogenous processes, which are fully natural and evolutionary optimized and thus, the delivery systems and conjugates are able to deliver compounds with high efficiency, low toxicity and a broad range of application into target cells. The delivery systems and conjugates provided by the present invention allow the effective delivery of biologically active compounds into both cultured cells and living organisms, for research, therapeutic and diagnostic purposes. The conjugates provided by the present invention are designed to be degraded and therefore, not accumulate within the targeted cells. Thus, the delivery systems and the conjugates of the present invention provide at least a solution to the cytosol delivery problem in the art as well as a solution to the toxicity problems in the art that result from accumulation of non-metabolized or undegraded delivery vehicles/constructs in the targeted cell.

In a first aspect, the present invention relates to a delivery system for delivery of a compound into a cell comprising or consisting of at least one conjugate comprising, essentially consisting of or consisting of:
(a) at least one module (a) that mediates cell targeting and facilitates cellular uptake,
(b) at least one module (b) that facilitates transport to the endoplasmic reticulum (ER),
(c) at least one module (c) that mediates translocation from the ER to the cytosol, and
(d) at least one compound (d),
   wherein the at least one module (a), the at least one module (b), the at least one module (c), and the at least one compound (d) are linked to each other in any arrangement. The delivery systems of the present invention optionally comprise a nuclear localization signal.

In a second aspect, the present invention relates to a delivery system for delivery of a compound into a cell comprising or consisting of at least one conjugate comprising, essentially consisting of or consisting of:
(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
   wherein at least two of the at least one module (a), the at least one module (b), and the at least one module (c) are comprised or contained within a multi-module protein or peptide, and wherein the multi-module protein or peptide, any remaining at least one module (a), at least one module (b), and at least one module (c) that are not comprised or contained within the multi-module protein or peptide, and the at least one compound (d) are linked to each other in any arrangement. The conjugates of the present invention optionally comprise a nuclear localization signal. Preferably, the multi-module protein or peptide comprises, consists essentially of, or consists of a contiguous protein or peptide or a protein that comprises, consists essentially of or contains at least two protein or peptide subunits or domains.

In a preferred embodiment of the second aspect, a conjugate of the present invention comprises, essentially consists of, or consists of or contains:
(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
   wherein the at least one module (a) and the at least one module (b) are comprised or contained within a [module (a)+module (b)] protein or peptide, and wherein the [module (a)+module (b)] protein or peptide, the at least one module (c), and the at least one compound (d) are linked to each other in any arrangement. The conjugates of the present invention optionally comprise a nuclear localization signal.

In a preferred embodiment of the second aspect, the present invention relates to a delivery system for delivery of a compound into a cell comprising or consisting of at least one conjugate comprising, essentially consisting of or consisting of:
(a) at least one module (a) that mediates cell targeting and facilitates cellular uptake,
b) at least one module (b) that facilitates transport to the ER,
(c) at least one module (c) that mediates translocation from the ER to the cytosol, and
(d) at least one compound (d),
   wherein the at least one module (b) and the at least one module (c) are comprised or contained within a [module (b)+module (c)] protein or peptide, and wherein the at least one module (a), the [module (b)+module (c)] protein or peptide, and the at least one compound (d) are linked to each other in any arrangement. The conjugates of the present invention optionally comprise a nuclear localization signal.

Preferably, within the conjugates of the present invention, the [module (b)+module (c)] protein or peptide is selected from the group consisting of a CX1a peptide (SEQ ID NO: 2), a CX2a peptide (SEQ ID NO: 3), a peptide comprising an amino acid sequence comprising SEQ ID NO: 4, a reduced toxicity or non-toxic toxin A-subunit comprising a module (b) protein or peptide, a reduced toxicity or non-toxic cholera toxin A-subunit, a reduced toxicity or non-toxic LT A-subunit, a reduced toxicity or non-toxic LT-II A-subunit, a reduced toxicity or non-toxic Pseudomonas exotoxin A Domain IA, and an acetylcholine esterase (AChE) protein or peptide comprising an amino acid sequence selected from the group consisting of In another preferred embodiment, a [module (b)+module (c)] protein or peptide comprises, consists essentially, or consists of an AChE protein or peptide comprising an amino acid sequence selected from the group consisting of SEQ ID NO: 292, SEQ ID NO: 293, SEQ ID NO: 294, SEQ ID NO: 295, SEQ ID NO: 296, SEQ ID NO: 297, SEQ ID NO: 298, SEQ ID NO: 299, (SEQ ID NO: 300, SEQ ID NO: 301, SEQ ID NO: 302, SEQ ID NO: 303, and SEQ ID NO: 304.

In a preferred embodiment of the second aspect, the present invention relates to a delivery system for delivery of a compound into a cell comprising or consisting of at least one conjugate comprising, essentially consisting of or consisting of:
(a) at least one module (a) that mediates cell targeting and facilitates cellular uptake,
b) at least one module (b) that facilitates transport to the ER,
(c) at least one module (c) that mediates translocation from the ER to the cytosol, and
(d) at least one compound (d),
   wherein the at least one module (a) and the at least one module (c) are comprised or contained within a [module (a)+module (c)] protein or peptide, and wherein the [module (a)+module (c)] protein or peptide, the at least one module (b), and the at least one compound (d) are linked to each other in any arrangement. The conjugates of the present invention optionally comprise a nuclear localization signal.

In a preferred embodiment of the second aspect, the present invention relates to a delivery system for delivery of a compound into a cell comprising or consisting of at least one conjugate comprising, essentially consisting of or consisting of:
(a) at least one module (a) that mediates cell targeting and facilitates cellular uptake,
b) at least one module (b) that facilitates transport to the ER,
(c) at least one module (c) that mediates translocation from the ER to the cytosol, and
(d) at least one compound (d),
   wherein the at least one module (a), the at least one module (b), and the at least one module (c) are comprised or contained within a [module (a)+module (b)+module (c)] protein or peptide, and wherein the [module (a)+module (b)+module (c)] protein or peptide, and the at least one compound (d) are linked to each other in any arrangement. The conjugates of the present invention optionally comprise a nuclear localization signal.

Preferably, within the conjugates of the present invention, the multi-module protein or peptide comprising, consisting essentially of, consisting of or containing the at least one module (a), the at least one module (b), and the at least one module (c) is selected from the group consisting of a non-toxic or reduced toxicity holo-toxin, a non-toxic or reduced toxicity ricin holo-toxin, a non-toxic ricin holo-toxin wherein in the ricin A subunit has an R180H mutation (SEQ ID NO: 1), a non-toxic or reduced toxicity Shiga holo-toxin, a non-toxic or reduced toxicity abrin holo-toxin, a non-toxic or reduced toxicity modeccin, a non-toxic or reduced toxicity viscumin, a non-toxic or reduced toxicity volkensin, a non-toxic or reduced toxicity cholera toxin, a non-toxic or reduced toxicity heat-labile enterotoxin, a non-toxic or reduced toxicity E. coli heat-labile enterotoxin, a non-toxic or reduced toxicity Pseudomonas exotoxin A, and a non-toxic or reduced toxicity pertussis toxin.

In a preferred embodiment of the second aspect, the present invention relates to a conjugate of the delivery system of the invention.

In a third aspect, the present invention relates to methods of preparing a delivery system or conjugate of the invention.

In a fourth aspect, the present invention relates to the use of the delivery system or conjugate of the invention as a pharmaceutical.

In a fifth aspect, the present invention relates to a pharmaceutical composition comprising the delivery system or conjugate of the present invention and a pharmaceutically acceptable excipient, carrier, and/or diluent.

In a sixth aspect, the present invention relates to the use of a delivery system or conjugate of the invention as a diagnostic reagent.

In a seventh aspect, the present invention relates to a use of the delivery system or conjugate of the invention for the manufacture of a medicament.

In an eighth aspect, the present invention relates to a method of delivering the compound (d) to a cell using the delivery system or conjugate of the invention.

In a ninth aspect, the present invention relates to a method of delivering the compound (d) to an organism using the delivery system or conjugate of the invention.

In a tenth aspect, the present invention relates to a method of delivering the compound (d) to a patient using the delivery system or conjugate of the invention.

### BRIEF DESCRIPTION OF THE DRAWINGS

FIG. 1 (A) to (D). (A), (B), (C), and (D) contain preferred embodiments of the conjugate of the present invention. The modules, or the modules and the compound may be linked to each other either covalently, non-covalently, via an adapter molecule or via a linker molecule that optimally comprises an adapter molecule.

FIGS. 2 (A and B). Detailed drawing of conjugate R-AK-CX described in Example 1. (A) illustrates a conjugate of the present invention, in which the cell targeting/uptake peptide [module (a)] is ricin toxin subunit B, the ERAD targeting/sorting peptide [module (c)] is from COX2, the ER targeting peptide [module (b)] is AKDEL, and the cargo [compound (d)] is an siRNA. The RTb is connected by a biodegradable disulfide bond to the N-terminus of the linkage peptide which carries modules (c) and (b) at the carboxy end. The siRNA cargo is linked, via the 5′-end of the sense strand containing a biodegradable (reducible) disulfide bond and an aminolinker, to the linkage peptide through an adapter derived from succinimidyl 4-formylbenzoate. The connection is made through a stable oxime bond generated by reaction of the formyl group with the aminooxy group of the branch point N-beta-aminooxyacetyl L-diaminopropionyl residue. The (SG)₃ units function as spacers to ensure that the various modules do not interfere with one another. (B) Illustrates the same molecule as described in FIG. 2 (A), but which includes a fluorescent dye at the 5′-end of the sense strand of the siRNA, to allow detection of the siRNA once it is released into the cytosol of the cell.

FIG. 3 (A) to (E). (A) illustrates a conjugate according to the present invention, wherein the modules and compound (d) are linked to each other in the following arrangement: module (a) is covalently linked to module (c) via a peptide linker molecule that comprises a cysteine side chain as branch point and a cleavage site upstream of the branch point, module (c) is covalently linked to module (b), and compound (d) is covalently linked via a disulfide-linkage to the cysteine side chain. (B) illustrates a conjugate according to the present invention, wherein the modules and compound (d) are linked to each other in the following arrangement: module (a) is covalently linked to module (c) via a first peptide linker molecule which comprises a cysteine side chain as branch point and a cleavage site upstream of the branch point, module (c) is covalently linked to module (b) via a second peptide linker molecule, and compound (d) is covalently linked via a disulfide-linkage to the cysteine side chain of the branch point. (C) illustrates another preferred embodiment, wherein compound (d) is linked via an enzymatic cleavage site instead of a disulfide-linkage to a cysteine side chain. Preferably, module (a) is cleaved off of the conjugate in the endosome or TGN, whereby making module (b) available for cellular receptors or other cellular proteins that bind to cellular receptors and then facilitate further transport to the ER. (D) illustrates a conjugate according to the present invention, wherein the at least one module (a), the at least one module (b), the at least module (c) and the at least one compound (d) are linked to each other in the following arrangements: the at least one module (a) is covalently linked to the at least one module (c) via a peptide linker molecule which comprises a cysteine side chain as a branch point and a cleavage site upstream of the branch point, the at least one module (c) is covalently linked to the at least one module (b) and the at least one compound (d) is non-covalently linked to the branch point via an ionic (electrostatic) linkage to DRBD that is covalently linked via a disulfide-linkage to the cysteine side chain. (E) illustrates a conjugate according to the present invention, wherein the modules and the compound are linked to each other in the following arrangement or combination: module (a) is covalently linked to module (c) via a peptide linker molecule which comprises a cysteine side chain as branch point and a cleavage site upstream of the branch point, module (c) is covalently linked to module (b) via a peptide linker molecule and compound (d) is non-covalently linked to the branch point via an ionic linkage to DRBD that is covalently linked via a disulfide-linkage to the cysteine side chain.

FIG. 4. Illustrates a conjugate of the present invention, in which module (a) is the non-toxic ricin toxin subunit B, RTb, the module (b) does not exist as a separate module but is part of RTb and module (c) does not exist as a separate module but is provided by part of RTb. Generally, 1-4 siRNAs as compound(s) (d) can be coupled to each RTB molecule via accessible amino groups such as those on lysine side chains plus the N-terminal amino group. The construct depicted in this Figure is referred to as DARE™ 1.01/DARE-R1/RTB-siRNA (via Lys). Briefly, the free thiol at Cys-4 is first inactivated by treatment with N-ethylmaleimide and the RTb is activated by reaction with an excess of a bifunctional crosslinker, e.g., sulfo-LC-SMPT, that contains an activated disulfide. Treatment of this intermediate with siRNA with a free thiol on the 5′-terminus of the antisense strand generates the conjugate illustrated by a simple disulfide exchange reaction. The location and number of siRNA coupling is not limited to the example shown in this Figure. Since RTB is activated with an excess of the bifunctional crosslinker sulfo-LC-SPDP (or sulfo-LC-SMPT), several molecules of siRNA per RTB monomer can be added. Separation of the entities with multiple siRNAs attached can be done by anion-exchange HPLC. The “N”s in the figure are only exemplary and do not represent actual locations of free amino side groups (except for the N-terminus).

FIG. 5. Illustrates a conjugate of the present invention, in which module (a) is the non-toxic ricin toxin subunit B, RTb, the module (b) does not exist as a separate module but is part of RTb and module (c) does not exist as a separate module but is provided as part of RTb. The cargo, compound (d), is an siRNA directly coupled via the 5′-end of the sense strand to the cysteine residue at position 4 of the RTb molecule through a biodegradable (reducible) disulfide bond. The construct depicted in this Figure is referred to as DARE™ 1.02/DARE-R2/RTB-siRNA (via Cys).

FIGS. 6 (A and B). (A) illustrates a conjugate of the present invention, in which the cell targeting/uptake peptide, module (a), is ricin toxin subunit B, the ERAD targeting/sorting peptide, module (c), is from COX2, the ER targeting functionality of module (b) is provided by RTb, and the cargo, compound (d), is an siRNA. The RTb is connected by a biodegradable disulfide bond to a cysteine residue at the N-terminus of the linkage peptide which carries module (c) at the C-terminus. The siRNA cargo is linked, via the 5′-end of the sense strand containing a biodegradable (reducible) disulfide bond and an aminolinker, to the linkage peptide through an adapter derived from succinimidyl 4-formylbenzoate. The connection is made through a stable oxime bond generated by reaction of the formyl group with the aminooxy group of the branch point N-beta-aminooxyacetyl L-diaminopropionyl residue. The (SG)₃ units function as spacers to ensure that the various modules do not interfere with one another. The construct depicted in this Figure is referred to as DARE™-2.01/DARE-R-CX/RTB—Cox2-ERSTEL-siRNA (B) illustrates the same molecule as described in FIG. 6 (A) but the (SG)₃ spacers are replaced by PEG spacers. The synthesis is described in Example 2. The construct depicted in this Figure is referred to as DARE™-2.02/DARE-R-CXpeg/RTB-peg—Cox2-ERSTEL-siRNA.

FIG. 7. Illustrates a conjugate of the present invention, in which the cell targeting/uptake protein or peptide, module (a), is ricin toxin subunit B, the ERAD targeting/sorting peptide, module (c), is from COX2, the ER targeting peptide, module (b), is KDEL, and the cargo, compound (d), is an siRNA. The RTb is connected by a biodegradable disulfide bond to the N-terminus of the linkage peptide which carries modules (c) and (b) at the C-terminus. The siRNA cargo is linked via the 5′-end of the sense strand containing a biodegradable (reducible) disulfide bond and an aminolinker, to the linkage peptide through an adapter derived from succinimidyl 4-formylbenzoate. The connection is made through a stable oxime bond generated by reaction of the formyl group with the aminooxy group of the branch point N-beta-aminooxyacetyl L-diaminopropionyl residue. The (SG)₃ units function as spacers to ensure that the various modules do not interfere with one another. The construct depicted in this Figure is referred to as DARE™-2.03/DARE-R-AK-CX/RTB—Cox2-AKDEL-siRNA.

FIG. 8. Illustrates a conjugate of the present invention identical to that illustrated in FIG. 7, with the exception that module (c), the ERAD targeting peptide, is omitted. The construct depicted in this Figure is referred to as DARE™-2.04/DARE-R-AK/RTB-AKDEL-siRNA.

FIG. 9. Illustrates a conjugate of the present invention, in which the cell targeting/uptake peptide, module (a), is ricin toxin subunit B, the ERAD targeting/sorting peptide, module (c), is from Sgk1, and the ER targeting peptide, module (b), is KDEL, and the cargo, compound (d), is an siRNA. The RTb is connected by a biodegradable disulfide bond to a cysteine residue at the N-terminus of the linkage peptide which carries modules (b) and (c). The siRNA cargo is linked, via the 5′-end of the sense strand containing a biodegradable (reducible) disulfide bond and an aminolinker, to the linkage peptide through an adapter derived from succinimidyl 4-formylbenzoate. The connection is made through a stable oxime bond generated by reaction of the formyl group with the aminooxy group of the branch point N-beta-aminooxyacetyl L-diaminopropionyl residue. The (SG)₃ units function as spacers to ensure that the various modules do not interfere with one another. The construct depicted in this Figure is referred to as DARE™ 2.05/DARE-R-AK-SGK/RTB—Sgk1-AKDEL-siRNA.

FIGS. 10 (A and B). (A) illustrates a conjugate of the present invention, in which module (a) is a transferrin receptor binding peptide, module (b) is KDEL and module (c) is a Cox2 peptide. All three modules are linked as a contiguous peptide. The (SG)₃ units function as spacers to ensure that the various modules do not interfere with one another. Compound (d) is an siRNA. The siRNA cargo is linked, via the 5′-end of the sense strand containing a biodegradable (reducible) disulfide bond to a cysteine residue of the peptide, located between the two (SG)₃ spacers. The construct depicted in this Figure is referred to as DARE™-3.01a/DARE-T-AK-CX_NC/TfR—Cox2-AKDEL-siRNA (N→C). (B) illustrates a conjugate of the present invention, in which the modules are the same as in FIG. 10 (A) however the construct is such that both modules (a) and (b) have their C-termini free. Module (a) is connected via its N-terminus to the branch point N-beta-aminooxyacetyl L-diaminopropionyl residue via a disulfide bond formed from 2 cysteine residues. Compound (d) is an siRNA. The siRNA cargo is linked, via the 5′-end of the sense strand containing an aminolinker, to the linkage peptide through an adapter derived from succinimidyl 4-formylbenzoate. The connection is made through a stable oxime bond generated by reaction of the formyl group of the adapter with the aminooxy group of the branch point N-beta-aminooxyacetyl L-diaminopropionyl residue. The (SG)₃ units function as spacers to ensure that the various modules do not interfere with one another. The construct depicted in this Figure is referred to as DARE™-3.01b/DARE-T-AK-CX_CC/TfR—Cox2-AKDEL-siRNA (→C; →C).

FIG. 11. Illustrates a conjugate of the present invention, in which module (a) is a transferrin receptor binding peptide, module (b) is KDEL and module (c) is an Sgk1 peptide. All three modules are linked as a contiguous peptide, with module (c) at the N-terminus and module (b) at the C-terminus. The (SG)₃ units function as spacers to ensure that the various modules do not interfere with one another. Compound (d) is an siRNA and is linked via the 5′-end of the sense strand through a biodegradable (reducible) disulfide bond to a cysteine residue of the peptide, located between the two (SG)₃ spacers. The construct depicted in this Figure is referred to as DARE™-3.02/DARE-T-AK-SGK/Sgk1—TfR-AKDEL-siRNA.

FIG. 12. Illustrates a conjugate of the present invention in which module (a) is a transferrin receptor binding peptide, module (b) is KDEL and is C-terminally linked to module (a), and module (c) is IgM(μ). Module (a) is connected via its N-terminus to the branch point N-beta-aminooxyacetyl L-diaminopropionyl residue via a disulfide bond formed from 2 cysteine residues. Compound (d) is an siRNA and is linked, via the 5′-end of the sense strand containing an aminolinker, to the linkage peptide through an adapter derived from succinimidyl 4-formylbenzoate. The connection is made through a stable oxime bond generated by reaction of the formyl group of the adapter with the aminooxy group of the branch point N-beta-aminooxyacetyl L-diaminopropionyl residue. The (SG)₃ units function as spacers to ensure that the various modules do not interfere with one another. The construct depicted in this Figure is referred to as DARE™-3.03/DARE-T-AK-IgM/TfR-AKDEL—IgM(μ)-siRNA.

FIG. 13. Illustrates a conjugate with an identical configuration to the conjugate depicted in FIG. 12 with the exception that module (b), which is the KDEL motif in this example, is now at the C-terminus of module (c), which is the IgM(μ) sequence. The construct depicted in this Figure is referred to as DARE™-3.04/DARE-T-IgM-AK/TfR—IgM(μ)-AKDEL-siRNA.

FIG. 14. Illustrates a conjugate of the present invention, whereby 2 cargo molecules, 2 compounds (d), are attached via biodegradable disulfide bonds. The cell targeting/uptake peptide, module (a), is ricin toxin subunit B, and the ERAD targeting/sorting peptide, module (c), and the ER targeting peptide, module (b), can be any module (c) and module (b) of use in a conjugate of the invention, but are located at the C-terminus of the linkage peptide. Module (a), RTb, is connected via a biodegradable (reducible) disulfide bond to a cysteine residue at the N-terminus of the linkage peptide which contains two branch point N-beta-aminooxyacetyl L-diaminopropionyl residues that are separated by a dPEG₁₂ spacer. The cargo molecules, 2 compounds (d), are siRNAs, each of which is linked via the 5′-end of the sense strand containing an aminolinker, to the linkage peptide through an adapter derived from succinimidyl 4-formylbenzoate. The connection is made through a stable oxime bond generated by reaction of the formyl group of the adapter with the aminooxy groups of the 2 branch point N-beta-aminooxyacetyl L-diaminopropionyl residues. The synthesis of an exemplary construct, in which module (c) is a Cox2 peptide and module (b) is KDEL, is described in Example 19.

FIG. 15. Illustrates the preparative anion-exchange HPLC trace of the DARE™ 3.02 construct, DARE™-T-AK-SGK with fLuc-siRNA as cargo, as described in Example 20. Separation was performed on a 1 mL Resource Q column with a linear gradient elution from 0 to 0.8 M sodium bromide in 25 mM Tris-HCl buffer, pH 7.4 containing 6 M urea during 60 min at a flow rate of 3 mL/min. The column effluent was monitored by UV at 260 and 550 nm. The x-axis is time in min and the y-axis is absorbance at 260 nm in mAU. The first peak is the desired DARE™ 3.02 construct.

FIG. 16. Illustrates the preparative anion-exchange HPLC trace of the DARE™ 3.02 construct, DARE™-T-AK-SGK with GAPDH-siRNA as cargo, as described in Example 20. Separation was performed on a 1 mL Resource Q column with a linear gradient elution from 0 to 0.8 M sodium bromide in 25 mM Tris-HCl buffer, pH 7.4 containing 6 M urea during 60 min at a flow rate of 3 mL/min. The column effluent was monitored by UV at 260 and 550 nm. The x-axis is time in min and the y-axis is absorbance at 260 nm in mAU. The first peak is the desired DARE™ 3.02 construct.

FIG. 17. Shown are PAGE analyses of the HPLC purified DARE™ 3.02 constructs with fLuc and GAPDH siRNA cargoes as described in Example 20. 15% PAGE gel, 8×6.5 cm, run for 1-1.5 h at 220 V and 25 mA with Tris-borate running buffer containing 6 M urea.

FIG. 18. MALDI-TOF mass spectrum of HPLC purified DARE™ 3.02 construct with fLuc-siRNA cargo (see Example 20). The construct is not completely stable to the MS conditions such that only a weak molecular ion with an m/z in the region of the calculated mass of 20544 Da can be observed. The observed main peak at m/z of 6830 is due to the antisense strand of the fLuc-siRNA (calculated mass 6827 Da), while the broad peak centered at m/z ˜13700 is due to the sense strand conjugated to the peptide.

FIG. 19. MALDI-TOF mass spectrum of HPLC purified DARE™ 3.02 construct with GAPDH-siRNA cargo (see Example 20). The construct is not completely stable to the MS conditions such that only a weak molecular ion with an m/z in the region of the calculated mass of 20577 Da can be observed. The observed main peak at m/z of 6799 is due to the antisense strand of the GAPDH-siRNA (calculated mass 6796 Da), while the broad peak centered at m/z ˜13800 is due to the sense strand conjugated to the peptide (calculated mass 13781 Da).

FIG. 20A. Elution profile of preparative gel filtration purification of crude DARE 2.03, viz. RTB-COX2-KDEL-siRNA (Gapdh). HiLoad 16/60 Superdex 75 prep grade column eluted at 1 mL/min with sterile PBS, pH 7.4. UV/VIS monitoring performed at 260, 285 and 550 nm. Peak 1 eluting at 55 min corresponds to the desired product. Peak 2 at 66 min is unreacted delivery carrier (RTB-COX2-KDEL) plus unreacted adapter-siRNA (Gapdh). The peak at 81 min corresponds to some excess antisense strand RNA.

FIG. 20B. Native PAGE of the peaks 1 and 2 from the gel filtration purifications of RTB-COX2-KDEL-siRNA (Gapdh) and RTB-COX2-KDEL-siRNA (Luc) with starting materials as markers. 20% pre-cast polyacrylamide gel, 8.0×6.5 cm and 1 mm thick, run for 1 h at 220 V and 25 mA with 50 mM Tris-borate, 1 mM EDTA, pH 8.3, running buffer. Top picture shows band detection by UV, lower picture shows band detection by “stains-all”. Lane 1 is peak 1 from the DARE-2.03-Gapdh purification showing product band at top plus an siRNA dimer impurity low down. Lane 2 is peak 2 from the DARE-2.03-Gapdh purification and shows unreacted RTB-COX2-KDEL high up (faint band by “stains-all”) plus unreacted adapter Gapdh-siRNA. Lane 3 is peak 1 from the DARE-2.03-Luc purification showing product band at top plus an siRNA dimer impurity low down. Lane 4 is peak 2 from the DARE-2.03-Luc purification and shows unreacted RTB-COX2-KDEL high up (faint band by “stains-all”) plus unreacted adapter Luc-siRNA. Lane 5 shows the delivery carrier marker, RTB-COX2-KDEL, high up on the gel as a faint band detected by “stains-all”. Lanes 6 & 7 show the adapter Gapdh-siRNA and adapter Luc-siRNA markers respectively; the antisense strand contaminant in the Gapdh-siRNA is clearly visible at the bottom of the gel as are the dimer siRNA impurities in both siRNAs at the position of the contaminant bands in lanes 1 and 3 respectively.

FIG. 20C. Native PAGE of DTT treated DARE 2.03-siRNA-Gapdh and DARE 2.03-siRNA-Luc plus controls and markers. 20% pre-cast polyacrylamide gel, 8.0×6.5 cm and 1 mm thick, run for 1 h at 220 V and 25 mA with 50 mM Tris-borate, 1 mM EDTA, pH 8.3, running buffer. Top picture shows band detection by UV, lower picture shows band detection by “stains-all”. Lane 1 is untreated DARE 2.03-Gapdh (RTB-COX2-KDEL-siRNA-Gapdh), with the top band being the correct product. Lane 2 is DTT treated DARE 2.03-Gapdh, showing total loss of the top product band to give the siRNA band at the bottom plus a very faint band high up from the RTB; the COX2-KDEL fragment is too faint to be observed. Lane 3 is untreated DARE 2.03-Luc (RTB-COX2-KDEL-siRNA-Luc), with the top band being the correct product and the lower band an impurity. Lane 4 is DTT treated DARE 2.03-Luc, showing almost total loss of the top product band to give the siRNA band at the bottom plus a very faint band high up from the RTB; the COX2-KDEL fragment is too faint to be observed. Lane 5 is the adapter Gapdh-siRNA marker showing the antisense strand contaminant. Lane 6 is the adapter Luc-siRNA. Lane 7 is the modified Luc sense strand RNA marker. Lane 8 is the unmodified Luc antisense strand RNA marker.

FIG. 21A. Depicts preferred reactions schemes that can be used to connect two parts of the conjugates of the present invention. Panel (I) depicts the reaction between a first compound containing a primary amine with a second compound containing a sulfosuccinimidyl ester to generate a new compound via an amide bond. The second compound may be a bifunctional crosslinker such as sulfo-LC-SPDP, sulfo-LC-SMPT, sulfo-SMCC, sulfo-GMBS, sulfo-S-4FB or sulfo-S-HyNic for example. Panel (II) depicts the reaction between a first compound containing a thiol with a second compound containing a 2-pyridyldithio moiety to generate a new compound with a (biodegradable) disulfide linkage. The second compound may be a bifunctional crosslinker such as 3-(2-pyridyldithio)propionyl hydrazide (PDPH). Panel (III) depicts the reaction between a first compound containing a thiol with a second compound containing a maleimido moiety to generate a new compound via a stable thioether linkage. The second compound may be a bifunctional crosslinker such as sulfo-SMCC, sulfo-GMBS or M2C2H for example. Panel (IV) depicts the reaction between a first compound containing a thiol with a second compound containing an iodoacetyl moiety to generate a new compound via a stable thioether linkage. The second compound may be a bifunctional crosslinker such as sulfo-STAB. Panel (V) depicts the reaction between a first compound containing an aminooxy moiety with a second compound containing an aryl aldehyde to generate a new compound via an aryl oxime linkage. The reaction rate is greatly enhanced by addition of aniline.

FIG. 21B. Depicts preferred reactions schemes that can be used to connect two parts of the conjugates of the present invention. Panel (VI) depicts the reaction between a first compound containing an aryl hydrazine with a second compound containing an aryl aldehyde to generate a new compound via a bis-aryl hydrazone linkage. The reaction rate is greatly enhanced by addition of aniline. Panel (VII) depicts the copper (I) catalyzed “click-reaction” between a first compound containing an alkynyl moiety with a second compound containing an azido moiety to generate a new compound containing a stable 1,2,3-triazine linkage. Panel (VIII) depicts the Diels-Alder 4+2 cycloaddition reaction between a first compound containing a 1,3-diene moiety with a second compound containing a dienophile, in this case a maleimide, to generate a new compound containing a cyclohexene ring.

FIG. 22: AMF-COX2STEL-siRNA structure, n=ratio. Illustrated is a conjugate of the present invention, in which the ER and cell targeting/uptake protein or peptide, module [(a)+(b)], is AMF, the ERAD targeting/sorting peptide, module (c), is from COX2, and the cargo, compound (d), is an siRNA. The AMF is connected by a biodegradable disulfide bond to the N-terminus of the linkage peptide which carries module (c) at the C-terminus. The siRNA cargo is linked via the 5P-end of the sense strand containing a biodegradable (reducible) disulfide bond and an aminolinker, to the linkage peptide through an adapter derived from succinimidyl 4-formylbenzoate. The connection is made through a stable oxime bond generated by reaction of the formyl group with the aminooxy group of the branch point N-beta-aminooxyacetyl L-diaminopropionyl residue. The (SG)₃ units function as spacers to ensure that the various modules do not interfere with one another. The construct depicted in this Figure is referred to as DARE™ AMF—COX2STEL-siRNA.

FIG. 23: AMF-MYCIGMμ-siRNA structure, n=ratio. Illustrated is a conjugate of the present invention, in which the ER and cell targeting/uptake protein or peptide, module [(a)+(b)], is AMF, the ERAD targeting/sorting peptide, module (c), is from mycIgM(μ) and the cargo, compound (d), is an siRNA. The AMF is connected by a biodegradable disulfide bond to the N-terminus of the linkage peptide which carries module (c) at the C-terminus. The siRNA cargo is linked via the 5′-end of the sense strand containing a biodegradable (reducible) disulfide bond and an aminolinker, to the linkage peptide through an adapter derived from succinimidyl 4-formylbenzoate. The connection is made through a stable oxime bond generated by reaction of the formyl group with the aminooxy group of the branch point N-beta-aminooxyacetyl L-diaminopropionyl residue. The (SG)₃ units function as spacers to ensure that the various modules do not interfere with one another. The construct depicted in this Figure is referred to as DARE′ AMF—mycIgMu-siRNA.

FIG. 24. CTB-COX2STEL-siRNA structure, n=ratio. Illustrates a conjugate of the present invention, in which the ER and cell targeting/uptake protein or peptide, module [(a)+(b)], is cholera toxin subunit B, the ERAD targeting/sorting peptide, module (c), is from COX2, the ER targeting peptide and the cargo, compound (d), is an siRNA. The CTB is connected by a biodegradable disulfide bond to the N-terminus of the linkage peptide which carries module (c) the C-terminus. The siRNA cargo is linked via the 5′-end of the sense strand containing a biodegradable (reducible) disulfide bond and an aminolinker, to the linkage peptide through an adapter derived from succinimidyl 4-formylbenzoate. The connection is made through a stable oxime bond generated by reaction of the formyl group with the aminooxy group of the branch point N-beta-aminooxyacetyl L-diaminopropionyl residue. The (SG)₃ units function as spacers to ensure that the various modules do not interfere with one another. The construct depicted in this Figure is referred to as DARE™ CTB—COX2STEL-siRNA.

FIG. 25. CTB-mycIgMu-siRNA structure, n=ratio. Illustrates a conjugate of the present invention, in which the ER and cell targeting/uptake protein or peptide, module [(a)+(b)], is cholera toxin subunit B, the ERAD targeting/sorting peptide, module (c), is from mycIgM(μ) and the cargo, compound (d), is an siRNA. The CTB is connected by a biodegradable disulfide bond to the N-terminus of the linkage peptide which carries module (c) at the C-terminus. The siRNA cargo is linked via the 5′-end of the sense strand containing a biodegradable (reducible) disulfide bond and an aminolinker, to the linkage peptide through an adapter derived from succinimidyl 4-formylbenzoate. The connection is made through a stable oxime bond generated by reaction of the formyl group with the aminooxy group of the branch point N-beta-aminooxyacetyl L-diaminopropionyl residue. The (SG)₃ units function as spacers to ensure that the various modules do not interfere with one another. The construct depicted in this Figure is referred to as DARE™ CTB—myclgMu-siRNA.

FIG. 26. CTB-(-COX2STEL)-(-siRNA) structure, n=m=ratio. Illustrates a conjugate of the present invention, in which the ER and cell targeting/uptake protein or peptide, module [(a)+(b)], is cholera toxin subunit B, the ERAD targeting/sorting peptide, module (c), is from COX2 and the cargo, compound (d), is an siRNA. The CTB is connected by a biodegradable disulfide bond to the N-terminus of the linkage peptide which carries module (c) at the C-terminus. The siRNA cargo is linked via the 5′-end of the sense strand containing a biodegradable (reducible) disulfide bond to CTB. The construct depicted in this Figure is referred to as DARE™ CTB-(-COX2STEL)-(-siRNA).

FIG. 27. CTB-(-myclgMu)-(-siRNA) structure, n=m=ratio. Illustrates a conjugate of the present invention, in which the cell targeting/uptake protein or peptide, module [(a)+(b)], is cholera toxin subunit B, the ERAD targeting/sorting peptide, module (c), is from mycIgM(μ), and the cargo, compound (d), is an siRNA. The CTB is connected by a biodegradable disulfide bond to the N-terminus of the linkage peptide which carries module (c) at the C-terminus. The siRNA cargo is linked via the 5′-end of the sense strand containing a biodegradable (reducible) disulfide bond to CTB. The construct depicted in this Figure is referred to as DARE™ CTB-(-myclgMu)-(-siRNA).

FIG. 28. CTB-COX2STEL-siRNA structure, n=ratio. CTB has residual reduced SPDP. Illustrates a conjugate of the present invention, in which the ER and cell targeting/uptake protein or peptide, module [(a)+(b)], is cholera toxin subunit B, the ERAD targeting/sorting peptide, module (c), is from COX2 and the cargo, compound (d), is an siRNA. The CTB is connected by a biodegradable disulfide bond to the N-terminus of the linkage peptide which carries module (c) at the C-terminus. The siRNA cargo is linked via the 5′-end of the sense strand containing a biodegradable (reducible) disulfide bond and an aminolinker, to the linkage peptide through an adapter derived from succinimidyl 4-formylbenzoate. The connection is made through a stable oxime bond generated by reaction of the formyl group with the aminooxy group of the branch point N-beta-aminooxyacetyl L-diaminopropionyl residue. The (SG)₃ units function as spacers to ensure that the various modules do not interfere with one another. The construct depicted in this Figure is referred to as DARE™ CTB—COX2STEL-siRNA.

FIG. 29. CTB-MYCIgMu-siRNA structure, n=ratio. CTB has residual reduced SPDP. Illustrates a conjugate of the present invention, in which the cell targeting/uptake protein or peptide, module [(a)+(b)], is cholera toxin subunit B, the ERAD targeting/sorting peptide, module (c), is from mycIgM(μ) and the cargo, compound (d), is an siRNA. The CTB is connected by a biodegradable disulfide bond to the N-terminus of the linkage peptide which carries module (c) at the C-terminus. The siRNA cargo is linked via the 5′-end of the sense strand containing a biodegradable (reducible) disulfide bond and an aminolinker, to the linkage peptide through an adapter derived from succinimidyl 4-formylbenzoate. The connection is made through a stable oxime bond generated by reaction of the formyl group with the aminooxy group of the branch point N-beta-aminooxyacetyl L-diaminopropionyl residue. The (SG)₃ units function as spacers to ensure that the various modules do not interfere with one another. The construct depicted in this Figure is referred to as DARE™ CTB—myclgMu-siRNA.

FIG. 30. CTB-CTA2-siRNA structure, Illustrates a conjugate of the present invention, in which the cell targeting/uptake protein or peptide, module [(a)+(b)], is cholera toxin subunit B, and the cargo, compound (d), is an siRNA. The CTB is non-covalently complexed with an N-terminal modified version of the natural CTA2 peptide (this has a natural KDEL sequence at the C-terminus and, thus, also comprises a module (b)), which connects through a stable triazole linkage to the 5′-end of the siRNA cargo. The connection is made through a [3+2] cycloaddition reaction between the alkynyl moiety of the propargylglycyl group on CTA2 with the azido group on the 5′-aminolinker of the siRNA using click chemistry conditions. The 5′-aminolinker contains a biodegradable (reducible) disulphide bond. The construct depicted in this Figure is referred to as DARE™ CTB—CTA2-siRNA.

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention. Unless defined otherwise, all technical and scientific terms used herein generally have the same meanings as commonly understood by one of ordinary skill in the art to which this invention belongs. Generally, the nomenclature used herein and the laboratory procedures in cell culture, molecular genetics, organic chemistry, and nucleic acid chemistry and hybridization are those well known and commonly employed in the art. Standard techniques are used for nucleic acid and peptide synthesis. The techniques and procedures are generally performed according to conventional methods in the art and various general references [e.g., 3], which are provided throughout this document. The nomenclature used herein and the laboratory procedures used in analytical chemistry and organic syntheses described below are those well known and commonly employed in the art. Standard techniques or modifications thereof are used for chemical syntheses and chemical analyses.

Preferably, the terms used herein are defined as previously described [4].

The articles “a” and “an” are used herein to refer to one or to more than one (i.e. to at least one) of the grammatical object of the article. By way of example, “an element” means one element or more than one element.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Several documents are cited throughout the text of this specification. Each of the documents cited herein (including all patents, patent applications, scientific publications, manufacturer's specifications, instructions, GenBank Accession Number sequence submissions etc.), whether supra or infra, is hereby incorporated by reference in its entirety. Nothing herein is to be construed as an admission that the invention is not entitled to antedate such disclosure by virtue of prior invention.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments that combine the explicitly described embodiments with any number of the disclosed and/or preferred elements. Furthermore, any permutations and combinations of all described elements in this application should be considered disclosed by the description of the present application unless the context indicates otherwise.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The sequences on a DNA strand that are located 5′ to a reference point on the DNA are referred to as “upstream sequences”; sequences on a DNA strand which are 3′ to a reference point on the DNA are referred to as “downstream sequences.”

A “polynucleotide” means a single strand or parallel and anti-parallel strands of a nucleic acid. Thus, a polynucleotide may be either a single-stranded or a double-stranded nucleic acid.

The term “nucleic acid” typically refers to a polynucleotide. Preferably, the nucleic acid of the conjugate of the present invention is single stranded or double stranded DNA, single stranded or double stranded RNA, siRNA, tRNA, mRNA, micro RNA (miRNA), small nuclear RNA (snRNA), small hairpin RNA (shRNA), morpholino modified iRNA (as described by Manoharan et al. in US2010/0076056 and U.S. Pat. No. 7,745,608), anti-gene RNA (agRNA), or the like.

“Homologous” as used herein, refers to the subunit sequence similarity between two polymeric molecules, e.g., between two nucleic acid molecules, e.g., two DNA molecules or two RNA molecules; or between two peptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 5′ATTGCC3′ and 5′TATGGC3′ share 50% homology.

As used herein, “homology” is used synonymously with “identity.” The determination of percent identity between two nucleotide or amino acid sequences can be accomplished using a mathematical algorithm. For example, a mathematical algorithm useful for comparing two sequences is the algorithm of Karlin and Altschul, 1990 [5], modified as in Karlin and Altschul, 1993 [6]. This algorithm is incorporated into the NBLAST and XBLAST programs of Altschul, et al., 1990 [7], and can be accessed, for example at the National Center for Biotechnology Information (NCBI) world wide web site having the universal resource locator “http://www.ncbi.nlm.nih.gov/BLAST/”. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al., 1997 [8]. Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used. See http://www.ncbi.nlm.nih.gov.

The percent identity between two sequences can be determined using techniques similar to those described above, with or without allowing gaps. In calculating percent identity, typically exact matches are counted.

A “protein” according to the present invention refers to a chain of amino acid residues which may be naturally occurring or derivatives of naturally occurring amino acid residues and which are preferably linked via peptide bonds, wherein the protein consists of at least 251 amino acid residues or amino acid residue derivatives.

A “peptide” according to the present invention refers to a chain of amino acid residues which may be naturally occurring or derivatives of naturally occurring amino acid residues and which are preferably linked via peptide bonds, wherein the peptide consists of not more than 250 amino acid residues or amino acid residue derivatives. Preferably, a peptide for use in the present invention is between 10 and 250 amino acid residues or amino acid residue derivatives in length. More preferably, a peptide for use in the present invention is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 amino acids in length.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine.

As used herein, amino acids are represented by the full name thereof, by the three letter code corresponding thereto, or by the one-letter code corresponding thereto, as indicated in the following Table 1:

TABLE 1
Amino acids and their three letter and one letter codes.

| Full Name | Three Letter Code | One Letter Code |
|---|---|---|
| Alanine | Ala | A |
| Arginine | Arg | R |
| Asparagine | Asn | N |
| Aspartic Acid | Asp | D |
| Cysteine | Cys | C |
| Glutamic Acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

“Amino acid analogs” refer to compounds that have the same basic chemical structure as a naturally occurring amino acid, i.e., an alpha (α) carbon that is linked to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid.

“Amino acid mimetics” refer to chemical compounds that have a structure that is different from the general chemical structure of an amino acid, but that function in a manner similar to a naturally occurring amino acid.

The present invention also provides for conjugates comprising an analog of a protein or peptide as described herein. Analogs may differ from naturally occurring proteins or peptides by conservative amino acid sequence differences or by modifications that do not affect sequence, or by both. For example, conservative amino acid changes may be made, which although they alter the primary sequence of the protein or peptide, do not normally alter its function. Conservative amino acid substitutions typically include substitutions within the following groups: glycine, alanine; valine, isoleucine, leucine; aspartic acid, glutamic acid; asparagine, glutamine; serine, threonine; lysine, arginine; and phenylalanine, tyrosine.

The present invention also provides for conjugates comprising a modified protein or peptide. Modifications that do not normally alter primary sequence include in vivo or in vitro chemical derivatization of proteins and peptides, e.g., acetylation, or carboxylation. Also included in the present invention are modified proteins or peptides that are glycosylated, e.g., those made by modifying the glycosylation patterns of a protein or peptide during its synthesis and processing or in further processing steps; e.g., by exposing the protein or peptide to enzymes which affect glycosylation, e.g., mammalian glycosylating or deglycosylating enzymes. Also embraced by the present invention are proteins or peptides that have phosphorylated amino acid residues, e.g., phosphotyrosine, phosphoserine, or phosphothreonine.

It will be appreciated, of course, that the proteins and peptides of use in the conjugates of the present invention may incorporate amino acid residues that are modified without affecting activity. For example, the termini may be derivatized to include blocking groups, i.e. chemical substituents suitable to protect and/or stabilize the N- and C-termini from “undesirable degradation”, a term meant to encompass any type of enzymatic, chemical or biochemical breakdown of the compound at its termini which is likely to affect the function of the compound, i.e. sequential degradation of the compound at a terminal end thereof.

Blocking groups include protecting groups conventionally used in the art of peptide chemistry that will not adversely affect the in vivo activities of the peptide. For example, suitable N-terminal blocking groups can be introduced by alkylation or acylation of the N-terminus Examples of suitable N-terminal blocking groups include C₁-C₅ branched or unbranched alkyl groups, acyl groups such as formyl and acetyl groups, as well as substituted forms thereof, such as the acetamidomethyl (Acm), Fmoc or Boc groups. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not incorporated, include esters, ketones or amides. Ester or ketone-forming alkyl groups, particularly lower alkyl groups such as methyl, ethyl and propyl, and amide-forming amino groups such as primary amines (—NH₂), and mono- and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without affect on peptide activity.

Other modifications can also be incorporated without adversely affecting the activity and these include, but are not limited to, substitution of one or more of the amino acids in the natural L-isomeric form with amino acids in the D-isomeric form. Thus, the protein or peptide of use in a conjugate of the present invention may include one or more D-amino acid residues, or may comprise amino acids that are all in the D-form. Retro-inverso forms of proteins or peptides in accordance with the present invention are also contemplated, for example, inverted peptides in which all amino acids are substituted with D-amino acid forms.

Acid addition salts of the proteins or peptides of use in a conjugate of the present invention are also contemplated as functional equivalents. Thus, a protein or peptide in accordance with the present invention that is treated with an inorganic acid such as hydrochloric, hydrobromic, sulfuric, nitric, phosphoric, hexafluorophosphoric, tetrafluoroboric, and the like, or an organic acid such as an acetic, propionic, glycolic, pyruvic, oxalic, malic, malonic, succinic, maleic, fumaric, tataric, citric, benzoic, trifluoroacetic, cinnamic, mandelic, methanesulfonic, ethanesulfonic, p-toluenesulfonic, salicyclic and the like, provides a water soluble salt of the peptide that is suitable for use in the conjugates of the present invention.

Also included are proteins and peptides that have been modified using ordinary molecular biological techniques so as to improve their resistance to proteolytic degradation or to optimize solubility properties or to render them more suitable as a therapeutic agent [e.g., when used as compound (d) in the conjugates of the invention]. Analogs of such peptides include those containing residues other than naturally occurring L-amino acids, e.g., D-amino acids or non-naturally occurring synthetic amino acids.

In addition, proteins and peptides that have been modified using ordinary molecular biological techniques so as to increase their susceptibility to proteolytic degradation [e.g., when used as modules (a), (b) and/or (c) in the conjugates of the invention] are also of use in the conjugates of the present invention. Preferably, the proteolytically susceptible protein or peptide comprises an ubiquitination site or motif. For the identification of such motifs see http://iclab.life.nctu.edu.tw/ubipred/[9, 10]. In a preferred embodiment, a module (a), module (b), or module (c) protein or peptide of use in the conjugate of the present invention comprises a ubiquitination site or motif, whereby a polyubiquitin chain is formed on the module (a), module (b), or module (c) protein or peptide. Preferably, the polyubiquitin chain is generated at lysine 11 or lysine 48 of ubiquitin [11, 12]. Preferably, at least four ubiquitin molecules are attached to a lysine residue(s) on the proteolytically susceptible module (a), module (b), or module (c) to increase its probability of recognition and degradation by the 26S-proteasome. In addition or alternatively, the proteolytically susceptible protein or peptide has been modified to add one or more lysine residues and/or have one or more of its amino acids substituted with one or more lysine residues to create a ubiquitination site within the proteolytically susceptible protein or peptide.

It should be understood that the proteins and peptides of use in the conjugates of the invention are not limited to products of any of the specific exemplary processes listed herein.

As used herein, a “variant” of a peptide or polypeptide of use in the present invention that comprises at least one change in its amino acid sequence, wherein the at least one change is an amino acid substitution, insertion, deletion, N-terminal truncation, C-terminal truncation, or any combination of these changes. A variant of the peptide or polypeptide of use in the present invention may comprise a change at more than one of its amino acid residues. In preferred embodiments, a variant usable in the present invention exhibits a total number of up to 200 (up to 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195 or 200) changes in the amino acid sequence (i.e. substitutions, insertions, deletions, N-terminal truncations, C-terminal truncations, and/or any combination thereof). The amino acid substitutions may be conservative or non-conservative. In preferred embodiments, a variant usable in the present invention differs from the protein or domain from which it is derived by up to 1, 2, 3, 4, 5, 6, 7, 8, 9, 10, 15, 20, 25, 30, 35, 40, 45, 50, 55, 60, 65, 70, 75, 80, 85, 90, 95, or 100 amino acid substitutions, preferably conservative amino acid changes. Variants may additionally or alternatively comprise deletions of amino acids, which may be N-terminal truncations, C-terminal truncations or internal deletions or any combination of these. Such variants comprising N-terminal truncations, C-terminal truncations and/or internal deletions are referred to as “deletion variants” or “fragments” in the context of the present application. The terms “deletion variant” and “fragment” are used interchangeably herein. A deletion variant may be naturally occurring (e.g. splice variants) or it may be constructed artificially, preferably by genetic engineering means, using recombinant DNA techniques.

A “conjugate” according to the present invention refers to the physical association of the compound (d) of interest (for example, a nucleic acid molecule or a peptide) with the modules (a), (b) and (c). In some embodiments, “conjugate” refers to the non-covalent association (e.g. electrostatic interaction, hydrogen bonding interaction or hydrophobic interaction) or covalent association of the afore-mentioned components. In other embodiments, all of the components of the conjugate may be covalently attached to each other, while in other embodiments, only a subset of the components are covalently attached to each other.

“Delivery” according to the present invention refers to a process by which the compound is transported into a cell, e.g. preferably into the cytosol (cytoplasm) of a cell, or into a cell organelle, preferably the nucleus.

A “compound” in the context of the present invention refers to a biologically active compound, i.e., a compound having the potential to react with biological components. More particularly, the compounds of use in the present invention are designed to change the natural cellular processes associated with a living cell. For purposes of this specification, a natural cellular process is a process that is associated with a cell before delivery of a compound that is biologically active. In the present invention, the cellular production of, or inhibition of a material, such as a protein or an mRNA, caused by the compound of the invention that is delivered to the cell, in vivo or in vitro, is an example of a delivered compound that is biologically active. Pharmaceuticals, peptides, proteins, and nucleic acids, cytotoxic agents, radioactive agents, and other therapeutic or diagnostic moieties are examples of compounds of the present invention.

As used herein, a “biologically active compound” is a biological molecule in a form in which it exhibits a property by which it is characterized. A functional enzyme, for example, is one which exhibits the characteristic catalytic activity by which the enzyme is characterized.

In the context of the present invention, the term “linked” means that the modules and the compound are physically attached to each other or associated with each other. In some embodiments, “linked” refers to a non-covalent association (e.g., electrostatic interaction, hydrogen bonding interaction or hydrophobic interaction) or covalent association of the afore-mentioned components. In other embodiments, all of the components may be covalently attached to each other, while in other embodiments, only a subset of the components are covalently attached to each other.

The term “linked to each other in any arrangement” further means that the modules and the compound can be linked linearly and/or non-linearly with each other, and in equal or different stoichiometries to each other.

The phrase “module that mediates cell targeting and facilitates cellular uptake also referred to herein as a “cell targeting module” or “module (a)”, refers in the context of the present invention to a chemical entity, e.g. a polypeptide or oligopeptide, preferably a polypeptide, capable of (i) specifically binding to the surface of a cell of interest, wherein preferably the cell is a vertebrate cell, more preferably a mammalian cell, such as a mouse, rat, goat, sheep, dog, cat, pig, cow, horse, primate, or human cell, etc., even more preferably a human cell, and (ii) mediating entry of the module and further components of the conjugate linked thereto into an intact cell via a natural process that might be an endocytosis process, which might be a receptor-mediated uptake, pinocytosis, phagocytosis, macropinocytosis or fluid-phase endocytosis allowing access to intracellular membrane-bound organelles or vesicles. Preferably, the module that mediates cell targeting and facilitates cellular uptake is taken up by the cell by a process that results in an intracellular membrane-bound vesicle, a membrane bound tubule or a membrane bound tubular vesicular structure. The structures, which are specifically bound by the module, are preferably cell surface receptors. One of ordinary skill in the art can readily assess whether a module mediates cell targeting and facilitates cellular uptake, e.g., by (i) labelling said module, for example, with a radioactive or fluorescent marker, (ii) incubating the labelled module with intact cells, preferably mammalian cells, for example human cells, and (iii) assessing whether the labelled module can be detected inside the cells, i.e. in an intracellular membrane-bound organelle or vesicle in the cytoplasm of the intact cells, e.g. by fluorescence microscopy [see for example, 13-15].

The phrase “module that facilitates the transport to the endoplasmic reticulum (ER)”, also referred to herein as an “ER targeting module” or “module (b)”, refers in the context of the present invention to a chemical entity, e.g. polypeptide or oligopeptide, preferable an oligopeptide, capable of mediating the transport of the module and further components of the conjugate linked thereto to the ER. The transport to the ER via the Golgi apparatus is in the opposite direction to the biosynthetic-secretory transport delivering molecules destined for secretion from the ER to the Golgi apparatus and further to the plasma membrane and is, therefore, also known as retrograde transport pathway to the ER. One of ordinary skill in the art can readily assess whether a module facilitates the transport to the ER, e.g., by (i) labelling said module, for example, with a radioactive or fluorescent marker, (ii) linking said labelled module to a module that mediates cell targeting and facilitates cellular uptake [module (a)], (iii) incubating both modules with intact cells, preferably mammalian cells, for example human cells, and (iv) assessing whether said labelled module can be detected in the ER of a cell, e.g. by fluorescence microscopy or assessment of its N-glycosylation status [14, 16].

The phrase “module that mediates translocation from the ER to the cytosol”, also referred to herein as an “ERAD targeting module” or “module (c)”, refers in the context of the present invention to a chemical entity, preferably a polypeptide or oligopeptide, capable of mediating the entry of the module and further components of the conjugate linked thereto, into the cytosol from the lumen of the ER, e.g. by acting as a substrate for ER-associated degradation (ERAD). The transport out of the ER into the cytosol is also known as retro-translocation. The ERAD pathway is a cellular pathway that normally targets misfolded or mis-glycosylated proteins for ubiquitination and subsequent degradation by a protein-degrading complex, called the proteasome. By exploiting the ERAD pathway using the module that mediates translocation from the ER to the cytosol, a conjugate of the present invention is able to deliver a compound to the cytoplasm, and whereby the cell targeting, ER targeting and ERAD targeting modules of the conjugate, if still remaining, will preferably be degraded by the proteosome. One of ordinary skill in the art can readily assess whether a module mediates translocation from the ER to the cytosol, e.g., by (i) labelling said module, for example, with a radioactive or fluorescent marker, (ii) linking said labelled module to a module that mediates cell targeting and facilitates cellular uptake [module (a)] and to a module that facilitates transport to the ER [module (b)], (iii) incubating the conjugated modules with intact cells, preferably mammalian cells, for example human cells, and (iv) assessing whether said labelled module can be detected in the cytosol of a cell and is degraded over time, presumably by the proteosome, e.g. by fluorescence microscopy or western blotting [See for example, 17].

One of ordinary skill in the art can also readily assess whether the modules (a), (b) and (c) carrying the above mentioned functionalities are able to deliver a compound into a cell, by (i) labelling the modules and the compound (d), for example, with different radioactive or fluorescent markers, (ii) linking the modules (a), (b) and (c) and the compound (d) to each other, (iii) incubating the conjugated modules and compound with intact cells, preferably mammalian cells, for example human cells, and (iv) assessing whether the compound (d) and modules can be detected in the cytosol of a cell, e.g. by fluorescence microscopy.

One of ordinary skill in the art can also use co-staining of the cells to determine the intracellular sorting of the module (a); of the modules (a) and (b); of the modules (a), (b) and (c); and of the modules (a), (b) and (c) and of the compound (d), i.e. of the conjugate. For example, cells comprising a module, modules, or the conjugate can be co-stained for intracellular compartments, e.g. endosomes, lysosomes, trans-golgi network, golgi apparatus, ER, caveolae and cytoplasm using immunohistochemistry as described below in Example 7.

In a first aspect, the present invention relates to a delivery system comprising or consisting of a conjugate for delivery of a compound into a cell, wherein the conjugate comprises, essentially consisting of or consists of:

(a) at least one module (a) that mediates cell targeting and facilitates cellular uptake, wherein the at least one module (a) is selected from the group consisting of a peptide (a1), a protein (a2), a toxin protein or peptide having reduced or no toxicity (a3), an A/B type toxin protein or peptide having reduced or no toxicity (a4), an A/B₅ type toxin protein or peptide having reduced or no toxicity (a5), an A/B type toxin subunit having reduced or no toxicity (a6), an A/B₅ type toxin subunit having reduced or no toxicity (a7), an A/B type holo-toxin having reduced or no toxicity (a8), an A/B₅ type holo-toxin having reduced or no toxicity (a9), an A/B type toxin B subunit (a10), an A/B₅ type toxin B-subunit (all), a non-toxic ricin holo-toxin (a12), a non-toxic ricin holotoxin wherein in the ricin A subunit has an R180H mutation (SEQ ID NO: 1) (a13), a mutant ricin holotoxin with reduced or no toxicity (a14), a ricin B-subunit (RTB) (a15), a ricin B-subunit peptide (a16), a cholera toxin (CT) B-subunit (CTB) (a17), a cholera toxin B-subunit peptide (a18), a non-toxic Shiga holo-toxin (a19), a mutant Shiga holo-toxin having reduced or no toxicity (a20), a Shiga toxin B-subunit (STB) (a21), a Shiga toxin B-subunit peptide (a22), an STx1a Shiga toxin B-subunit (a23), an Stx1b [Verotoxin (VT) 1b (VT1b)] Shiga toxin B-subunit (a24), an Stx1c (VT1c) Shiga toxin B-subunit (a25), an Stx1d (VT1d) Shiga toxin B-subunit (a26), an Stx2a (VT2a) Shiga toxin B-subunit (a27), an Stx2b (VT2b) Shiga toxin B-subunit (a28), an Stx2c (VT2c) Shiga toxin B-subunit (a29), an Stx2d (VT2d) Shiga toxin B-subunit (a30), an Stx2e (VT2e) Shiga toxin B-subunit (a31), an Stx2f (VT2f) Shiga toxin B-subunit (a32), an Escherichia coli heat labile enterotoxin (LT) B-subunit (a33), an LT-IIa B-subunit (a34), an LT-IIb B-subunit (a35), an Abrin-a B-subunit (a36), an Abrin-b B-subunit (a37), an Abrin-c B-subunit (a38), an Abrin-d B-subunit (a39), a Pertussis B-subunit (a40), a Modeccin B-subunit (a41), a Volkensin B-subunit (a42), a Viscumin B-subunit (a43), a Pseudomonas exotoxin A Domain IA (a44), an Escherichia coli subtilase cytotoxin B-subunit (a45), a Tetanus toxin C-fragment (a46), a hybrid AB toxin with reduced or no toxicity (a47), a hybrid ricin-abrin toxin with reduced or no toxicity (a48), a hybrid AB₅ toxin with reduced or no toxicity (a49), a hybrid LT-CT toxin with reduced or no toxicity (a50), a hybrid A1(LT1)-A2(CT)-B5(CT) toxin with reduced or no toxicity (a51), a hybrid SLT-ST toxin with reduced or no toxicity (a52), a hybrid A1(SLT)-A2(ST)-B5(ST) toxin with reduced or no toxicity (a53), an AMF (a54), an SUMF (a55), an HDL (a56), an LDL (a57), a holo-transferrin (a58), a TfR binding peptide (a59), an antibody (a60), an antibody fragment (a61), a TGN38/42 antibody (a62), a cation independent MPR antibody (a63), a cation dependent MPR antibody (a64), a Sortilin antibody (a65), a polymeric IgA receptor antibody (a66), a Wnt protein ligand or antibody (a67), a Wnt1 protein ligand or antibody (a68), an amyloid precursor protein (APP) ligand or antibody (a69), an apolipoprotein A-V ligand or antibody (a70), an Stx2g (VT2g) Shiga toxin B-subunit (a71), an Stx1a Shiga toxin B-subunit peptide (a72), an Stx1b (VT1b) Shiga toxin B-subunit peptide (a73), an Stx1c (VT1c) Shiga toxin B-subunit peptide (a74), an Stx1d (VT1d) Shiga toxin B-subunit peptide (a75), an Stx2a (VT2a) Shiga toxin B-subunit peptide (a76), an Stx2b (VT2b) Shiga toxin B-subunit peptide (a77), an Stx2c (VT2c) Shiga toxin B-subunit peptide (a78), an Stx2d (VT2d) Shiga toxin B-subunit peptide (a79), an Stx2e (VT2e) Shiga toxin B-subunit peptide (a80), an Stx2f (VT2f) Shiga toxin B-subunit peptide (a81), an Stx2g (VT2g) Shiga toxin B-subunit peptide (a82), a non-toxic STx1a Shiga holo-toxin (a83), a non-toxic Stx1b (VT1b) Shiga holo-toxin (a84), a non-toxic Stx1c (VT1c) Shiga holo-toxin (a85), a non-toxic Stx1d (VT1d) Shiga holo-toxin (a86), a non-toxic Stx2a (VT2a) Shiga holo-toxin (a87), a non-toxic Stx2b (VT2b) Shiga holo-toxin (a88), a non-toxic Stx2c (VT2c) Shiga holo-toxin (a89), a non-toxic Stx2d (VT2d) Shiga holo-toxin (a90), a non-toxic Stx2e (VT2e) Shiga holo-toxin (a91), a non-toxic Stx2f (VT2f) Shiga holo-toxin (a92), a non-toxic Stx2g (VT2g) Shiga holo-toxin (a93), a mutant STx1a Shiga holo-toxin having reduced or no toxicity (a94), a mutant Stx1b (VT1b) Shiga holo-toxin having reduced or no toxicity (a95), a mutant Stx1c (VT1c) Shiga holo-toxin having reduced or no toxicity (a96), a mutant Stx1d (VT1d) Shiga holo-toxin having reduced or no toxicity (a97), a mutant Stx2a (VT2a) Shiga holo-toxin having reduced or no toxicity (a98), a mutant Stx2b (VT2b) Shiga holo-toxin having reduced or no toxicity (a99), a mutant Stx2c (VT2c) Shiga holo-toxin having reduced or no toxicity (a100), a mutant Stx2d (VT2d) Shiga holo-toxin having reduced or no toxicity (a101), a mutant Stx2e (VT2e) Shiga holo-toxin having reduced or no toxicity (a102), a mutant Stx2f (VT2f) Shiga holo-toxin having reduced or no toxicity (a103), and a mutant Stx2g (VT2g) Shiga holo-toxin having reduced or no toxicity (a104).
(b) at least one module (b) that facilitates transport to the endoplasmic reticulum (ER), wherein the at least one module (b) is selected from the group consisting of an oligopeptide comprising one or more of the amino acid sequence X₁X₂X₃X₄ (SEQ ID NO: 5), wherein X₁ is E, H, K, N, P, Q, R or S, preferably K or R; X₂ is D, E, A, T, V, G, S or N, preferably D or E; X₃ is E or D, preferably E; X₄ is L or F, preferably L, and wherein optionally the N-terminus and/or C-terminus comprises 1 to 3 additional amino acid residues. Particularly preferred examples of module (b) are EDEL (SEQ ID NO: 6) (b1), HDEL (SEQ ID NO: 7) (b2), HEEL (SEQ ID NO: 8) (b3), KAEL (SEQ ID NO: 9) (b4), KDEF (SEQ ID NO: 10) (b5), KEDL (SEQ ID NO: 11) (b6), KEEL (SEQ ID NO: 12) (b7), KTEL (SEQ ID NO: 13) (b8), KVEL (SEQ ID NO: 14) (b9), NEDL (SEQ ID NO: 15) (b10), PDEL (SEQ ID NO: 16) (b11), PGEL (SEQ ID NO: 17) (b12), QEDL (SEQ ID NO: 18) (b13), QSEL (SEQ ID NO: 19) (b14), REDL (SEQ ID NO: 20) (b15), RNEL (SEQ ID NO: 21) (b16), RTDL (SEQ ID NO: 22) (b17), RTEL (SEQ ID NO: 23) (b18), ERSTEL (SEQ ID NO: 24) (b19), KDEL (SEQ ID NO: 25) (b20), AKDEL (SEQ ID NO: 26) (b21), PTEL (SEQ ID NO: 27) (b22), STEL (SEQ ID NO: 28) (b23), REDLK (SEQ ID NO: 29) (b24), and RDEL (SEQ ID NO: 30) (b25),
(c) at least one module (c) that mediates translocation from the ER to the cytosol, wherein the at least one module (c) is selected from the group consisting of a peptide (c1), a protein (c2), a C-terminal destabilizing oligopeptide (c3), a C-terminal destabilizing oligopeptide comprising, consisting essentially of, consisting of or containing an amino acid sequence selected from the group consisting of CL1 (SEQ ID NO: 31) (c4), CL2 (SEQ ID NO: 32) (c5), CL6 (SEQ ID NO: 33) (c6), CL9 (SEQ ID NO: 34) (c7), CL10 (SEQ ID NO: 35) (c8), CL11 (SEQ ID NO: 36) (c9), CL12 (SEQ ID NO: 37) (c10), CL15 (SEQ ID NO: 38) (c11), CL16 (SEQ ID NO: 39) (c12), SL17 (SEQ ID NO: 40) (c13), a COX2 peptide (c14), a COX2 peptide comprising, consisting essentially of, consisting of or containing an amino acid sequence selected from the group consisting of SEQ ID NO: 41 (c15), SEQ ID NO: 42 (c16), SEQ ID NO: 43 (c17), SEQ ID NO: 44 (c18), SEQ ID NO: 45 (c19), SEQ ID NO: 46 (c20), SEQ ID NO: 47 (c21), and SEQ ID NO: 48 (c22), an IgM(μ) peptide (c23), an IgM(μ) peptide comprising, consisting essentially of, consisting of or containing an amino acid sequence selected from the group consisting of SEQ ID NO: 49 (c24), SEQ ID NO: 50 (c24), SEQ ID NO: 51 (c25), SEQ ID NO: 52 (c26), SEQ ID NO: 53 (c27), SEQ ID NO: 54 (c28), SEQ ID NO: 55 (c28), SEQ ID NO: 56 (c29), and SEQ ID NO: 57 (c30), an Sgk1 peptide (c31), an Sgk1 peptide comprising, consisting essentially of, consisting of or containing an amino acid sequence selected from the group consisting of SEQ ID NO: 58 (c32), SEQ ID NO: 59 (c33), SEQ ID NO: 60 (c34), SEQ ID NO: 61 (c35), SEQ ID NO: 62 (c36), SEQ ID NO: 63 (c37), SEQ ID NO: 64 (c38), SEQ ID NO: 65 (c39), SEQ ID NO: 66 (c40), SEQ ID NO: 67 (c41), SEQ ID NO: 68 (c42), SEQ ID NO: 69 (c43), SEQ ID NO: 70 (c44), SEQ ID NO: 71 (c45), SEQ ID NO: 72 (c46), SEQ ID NO: 73 (c47), SEQ ID NO: 74 (c48), SEQ ID NO: 75 (c49), SEQ ID NO: 76 (c50), and SEQ ID NO: 77 (c51), an MATα2 peptide (c52), an MATα2 peptide comprising, consisting essentially of, consisting of or containing an amino acid sequence selected from the group consisting of SEQ ID NO: 78 (c53), SEQ ID NO: 79 (c54), SEQ ID NO: 80 (c55), SEQ ID NO: 81 (c56), SEQ ID NO: 82 (c57), SEQ ID NO: 83 (c58), SEQ ID NO: 84 (c59), and SEQ ID NO:85 (c60), an MFα1 peptide (c61), an MFα1 peptide comprising, consisting essentially of, consisting of or containing an amino acid sequence selected from the group consisting of SEQ ID NO: 86 (c62), SEQ ID NO: 87 (c63), SEQ ID NO: 88 (c64), SEQ ID NO: 89 (c65), and SEQ ID NO: 90 (c66), a CPY peptide (c67), a CPY peptide comprising, consisting essentially of, consisting of or containing an amino acid sequence of SEQ ID NO: 91 (c68), a toxin protein or peptide having reduced or no toxicity (c69), an A/B type toxin protein or peptide having reduced or no toxicity (c70), an A/B5 type toxin protein or peptide having reduced or no toxicity (c71), a toxin subunit having reduced or no toxicity (c72), an A/B type toxin subunit having reduced or no toxicity (c73), an A/B5 type toxin subunit having reduced or no toxicity (c74), a mutated toxin A-subunit having reduced or no toxicity (c75), a non-toxic or reduced toxicity toxin A1-subunit (c76), a toxin B-subunit (c77), a mutated ricin toxin A-subunit (RTA) having reduced or no toxicity (c78), a mutated ricin toxin A1-subunit (RTA1) having reduced or no toxicity (c79), a ricin toxin B-subunit (RTB) (c80), a mutated cholera toxin A-subunit (CTA) having reduced or no toxicity (c81), a mutated cholera toxin A1-subunit (CTA1) having reduced or no toxicity (c82), a cholera toxin B-subunit (CTB) (c83), a mutated Shiga toxin (ST) A-subunit having reduced or no toxicity (c84), a mutated Shiga toxin A1-subunit (STA1) having reduced or no toxicity (c85), a Shiga toxin B-subunit (STB) (c86), a mutated Stx1a Shiga toxin A-subunit having reduced or no toxicity (c87), a mutated Stx1b (VT1b) Shiga toxin A-subunit having reduced or no toxicity (c88), a mutated Stx1c (VT1c) Shiga toxin A-subunit having reduced or no toxicity (c89), a mutated Stx1d (VT1d) Shiga toxin A-subunit having reduced or no toxicity (c90), a mutated Stx2a (VT2a) A-subunit having reduced or no toxicity (c91), a mutated Stx2b (VT2b) A-subunit having reduced or no toxicity (c92), a mutated Stx2c (VT2c) A-subunit having reduced or no toxicity (c93), a mutated Stx2d (VT2d) A-subunit having reduced or no toxicity (c94), a mutated Stx2e (VT2e) A-subunit having reduced or no toxicity (c95), a mutated Stx2f (VT2f) A-subunit having reduced or no toxicity (c96), a mutated Stx2g (VT2g) A-subunit having reduced or no toxicity (c97), an Stx1a Shiga toxin B-subunit (c98), an Stx1b (VT1b) Shiga toxin B-subunit (c99), an Stx1c (VT1c) Shiga toxin B-subunit (c100), an Stx1d (VT1d) Shiga toxin B-subunit (c101), an Stx2a (VT2a) Shiga toxin B-subunit (c102), an Stx2b (VT2b) Shiga toxin B-subunit (c103), an Stx2c (VT2c) Shiga toxin B-subunit (c104), an Stx2d (VT2d) Shiga toxin B-subunit (c105), an Stx2e (VT2e) Shiga toxin B-subunit (c106), a mutated Escherichia coli heat labile enterotoxin (LT) A-subunit (LT-A) having reduced or no toxicity (c107), a mutated LT-IIa A-subunit having reduced or no toxicity (c108), a mutated LT-IIa A-subunit peptide having reduced or no toxicity (c109), a mutated LT-IIb A-subunit having reduced or no toxicity (c110), an LT B-subunit (LT-B) (c111), an LT-IIa B-subunit (c112), an LT-IIb B-subunit (c113), a mutated Abrin-a A-subunit having reduced or no toxicity (c114), a mutated Abrin-b A-subunit having reduced or no toxicity (c115), a mutated Abrin-c A-subunit having reduced or no toxicity (c116), a mutated Abrin-d A-subunit having reduced or no toxicity (c117), a mutated pertussis A-subunit having reduced or no toxicity (c118), a pertussis B-subunit (c119), a mutated Modeccin A-subunit having reduced or no toxicity (c120), a Modeccin B-subunit (c121), a mutated Volkensin A-subunit having reduced or no toxicity (c122), a Volkensin B-subunit (c123), a mutated Viscumin A-subunit having reduced or no toxicity (c124), a Viscumin B-subunit (c125), a non-toxic Pseudomonas Exotoxin A holo-toxin (c126), a mutated Pseudomonas Exotoxin A having reduced or no toxicity (c127), a Pseudomonas Exotoxin A Domain II (c128), a mutated Escherichia coli subtilase cytotoxin A-subunit having reduced or no toxicity (c129), an Escherichia coli subtilase cytotoxin B-subunit (c130), a mutated Cinnamomin I toxin A-subunit having reduced or no toxicity (c131), a mutated Cinnamomin II toxin A-subunit having reduced or no toxicity (c132), a mutated Cinnamomin III toxin A-subunit having reduced or no toxicity (c133), a mutated ribosome-inactivating protein SNAI′ A-subunit having reduced or no toxicity (c134), a mutated Ebulin 1 ribosome-inactivating protein (ebu1) A-subunit having reduced or no toxicity (c135), a mutated type 2 ribosome-inactivating protein SNAIf A-subunit having reduced or no toxicity (c136), a mutated lectin [Q41358 (Q41358_SAMNI)] A-subunit having reduced or no toxicity (c137), a mutated ribosome-inactivating protein (AV1) A-subunit having reduced or no toxicity (c138), a mutated type 2 ribosome-inactivating protein Nigrin 1 A-subunit having reduced or no toxicity (c139), a mutated type 2 ribosome-inactivating protein Nigrin b A-subunit having reduced or no toxicity (c140), a mutated Bodinierin toxin A-subunit having reduced or no toxicity (c141), a mutated Porrectin toxin A-subunit having reduced or no toxicity (c142), a mutated cinphorin toxin A-subunit with reduced or no toxicity (c143), an α1-AT peptide (c144), an ASGPR H2a peptide (c145), a BACE457 peptide (c146), a CD3δ peptide (c147), a TCRα peptide (c148), a ΔF508 of CFTR peptide (c149), an HMG-CoA reductase peptide (c150), an IgK LCNS peptide (c151), a KAI1 (CD82) peptide (c152), an MHC class I peptide (c153), a Pael-R peptide (c154), a transthyretin (TTR) peptide (c155), a viral peptide (c156), an SV40 viral peptide (c157), a murine polyomavirus peptide (c158), a BK viral peptide (c159), a JC viral peptide (c160), a KI viral peptide (c161), a WU viral peptide (c162), a Merkel Cell polyomavirus peptide (c163), an Stx2f (VT2f) Shiga toxin B-subunit (c164), an Stx2g (VT2g) Shiga toxin B-subunit (c165), a Shiga toxin A1-subunit peptide (c166), an Stx1a Shiga toxin A1-subunit peptide (c167), an Stx1b (VT1b) Shiga toxin A1-subunit peptide (c168), an Stx1c (VT1c) Shiga toxin A1-subunit peptide (c169), an Stx1d (VT1d) Shiga toxin A1-subunit peptide (c170), an Stx2a (VT2a) Shiga toxin A1-subunit peptide (c171), an Stx2b (VT2b) Shiga toxin A1-subunit peptide (c172), an Stx2c (VT2c) Shiga toxin A1-subunit peptide (c173), an Stx2d (VT2d) Shiga toxin A1-subunit peptide (c174), an Stx2e (VT2e) Shiga toxin A1-subunit peptide (c175), an Stx2f (VT2f) Shiga toxin A1-subunit peptide (c176), an Stx2g (VT2g) Shiga toxin A1-subunit peptide (c177), a mutated Stx1a Shiga toxin A1-subunit having reduced or no toxicity (c178), a mutated Stx1b (VT1b) Shiga toxin A1-subunit having reduced or no toxicity (c179), a mutated Stx1c (VT1c) Shiga toxin A1-subunit having reduced or no toxicity (c180), a mutated Stx1d (VT1d) Shiga toxin A1-subunit having reduced or no toxicity (c181), a mutated Stx2a (VT2a) Shiga toxin A1-subunit having reduced or no toxicity (c182), a mutated Stx2b (VT2b) Shiga toxin A1-subunit having reduced or no toxicity (c183), a mutated Stx2c (VT2c) Shiga toxin A1-subunit having reduced or no toxicity (c184), a mutated Stx2d (VT2d) Shiga toxin A1-subunit having reduced or no toxicity (c185), a mutated Stx2e (VT2e) Shiga toxin A1-subunit having reduced or no toxicity (c186), a mutated Stx2f (VT2f) Shiga toxin A1-subunit having reduced or no toxicity (c187), a mutated Stx2g (VT2g) Shiga toxin A1-subunit having reduced or no toxicity (c188), a Shiga toxin B-subunit peptide (c189), an Stx1a Shiga toxin B-subunit peptide (c190), an Stx1b (VT1b) Shiga toxin B-subunit peptide (c191), an Stx1c (VT1c) Shiga toxin B-subunit peptide (c192), an Stx1d (VT1d) Shiga toxin B-subunit peptide (c193), an Stx2a (VT2a) Shiga toxin B-subunit peptide (c194), an Stx2b (VT2b) Shiga toxin B-subunit peptide (c195), an Stx2c (VT2c) Shiga toxin B-subunit peptide (c196), an Stx2d (VT2d) Shiga toxin B-subunit peptide (c197), an Stx2e (VT2e) Shiga toxin B-subunit peptide (c198), an Stx2f (VT2f) Shiga toxin B-subunit peptide (c199), an Stx2g (VT2g) Shiga toxin B-subunit peptide (c200), a c-myc tagged IgM(μ) peptide (201), and an acetyl choline esterase (AChE) peptide selected from the group consisting of SEQ ID NO: 280 (c202), SEQ ID NO: 281 (c203), SEQ ID NO: 282 (c204), SEQ ID NO: 283 (c205), SEQ ID NO: 284 (c206), SEQ ID NO: 285 (c207), SEQ ID NO: 286 (c208), SEQ ID NO: 287 (c209), SEQ ID NO: 288 (c210), and SEQ ID NO: 289 (c211), and (d) at least one compound (d), wherein the at least one compound (d) is selected from the group consisting of a protein (d1), a peptide (d2), an oligopeptide (d3), a nucleic acid (d4), an oligonucleotide (d5), a DNA molecule (d6), a single stranded DNA molecule (d7), a double stranded DNA molecule (d8), an RNA molecule (d9), a single stranded RNA molecule (d10), a double stranded RNA molecule (d11), an siRNA molecule (d12), a tRNA molecule (d13), an mRNA molecule (d14), a micro RNA (miRNA) molecule (d15), a small nuclear RNA (snRNA) molecule (d16), a small hairpin RNA (shRNA) molecule (d17), a morpholino modified iRNA molecule (d18), an anti-gene RNA (agRNA) molecule (d19), a zippered interfering RNA (ziRNA) (d20), an antisense RNA molecule (d21), a RISC component (d22), a DICER protein (d23), an Argonaute protein (d24), an Argonaute-related protein (d25), a TRBP (d26), a double stranded RNA binding domain protein (d27), a PACT protein (d28), a helicase (d29), a nuclease (d30), an antigen (d31), an NSP4 (d32), an Influenza nucleoprotein NP (d33), an LCMV glycoprotein 1 (d34), an hTRT (d35), a CYFRA 21-1 (d36), a p53 peptide (d37), a ras peptide (d38), a β-catenin (d39), a CDK4 (d40), a CDC27 (d41), an a actinin-4 (d42), a tyrosinase (d43), a TRP1/gp75 (d44), a TRP2 (d45), a gp100 (d46), a Melan-A/MART1 (d47), a ganglioside (d48), a PSMA (d49), an HER2 (d50), a WT1 (d51), an EphA3 (d52), an EGFR (d53), a CD20 (d54), a MAGE (d55), a BAGE (d56), a GAGE (d57), an NY-ESO-1 (d58), a Survivin (d59), a DARE enhancer (d60), a small molecule (d61), tamoxifen (d62), dexamethasone (d63), taxol (d64), paclitaxel (d65), cisplatin (d66), oxaliplatin (d67), carboplatin (d68), a therapeutic molecule (d69), an antibody (d70), an antibody fragment (d71), a peptoid (d72), a decoy oligonucleotide (d73), a diagnostic molecule (d74), an imaging molecule (d75), Herpes simplex virus thymidine kinase (HSV1-TK) (d76), a fluorochrome (d77), a quantum dot (d78), a (super-) (para-) magnetic nanoparticle (d79), a labelled antibody (d80), a labelled antibody fragment (d81), a molecular beacon (d82), a biosensor (d83), carbonic anhydrase (d84), an oligopeptide-based probe (d85), an oligopeptide-based probe for detection of protease activity (d86), a peptide-based fluorescent sensor (d87), a peptide-based fluorescent sensor of protein kinase activity (d88), a radioactively-labeled metabolite (d89), D2R (d90), a tumor suppressor protein (d91), a tumor suppressor peptide (d92), p53 (d93), p21 (d94), p15 (d95), BRCA1(d96), BRCA2 (d97), IRF-1 (d98), PTEN (d99), RB (d100), APC (d101), DCC (d102), NF-1 (d103), NF-2 (d104), WT-1 (d105), MEN I (d106), MEN-II (d107), zac1 (d108), p73 (d109), VHL (d110), MMAC1 (d111), FCC (d112), MCC (d113), an enzyme (d114), cytosine deaminase (d115), adenosine deaminase (d116), hypoxanthine-guanine phosphoribosyltransferase (d116), galactose-1-phosphate uridyltransferase (d117), phenylalanine hydroxylase (d118), glucocerebrosidase (d119), sphingomyelinase (d120), a-L-iduronidase (d121), glucose-6-phosphate dehydrogenase (d122), HSV thymidine kinase (d123), human thymidine kinase (d124), an interleukin (d125), a cytokine (d126), IL-1 (d127), IL-2 (d128), IL-3 (d129), IL-4 (d130), IL-5 (d131), IL-6 (d132), IL-7 (d133), IL-8 (d134), IL-9 (d135), IL-10 (d136), IL-11 (d137), IL-12 (d138), IL-13 (d139), IL-14 (d140), IL-15 (d141), P-interferon (d142), alpha-interferon (d143), beta-interferon (d144), gamma-interferon (d145), angiostatin (d146), thrombospondin (d147), endostatin (d148), METH-1 (d149), METH-2 (d150), GM-CSF (D151), G-CSF (d152), M-CSF (d153), tumor necrosis factor (d154), a cell cycle regulator (d155), p27 (d156), p16 (d157), p21 (d158), p57 (d159), p18 (d160), p73 (d161), p19 (d162), p15 (d163), E2F-1 (d164), E2F-2 (d165), E2F-3 (d165), p107 (d166), p130 (d167), E2F-4 (d168), a transcription factor (d169), or a small molecule that regulates transcription (d170), wherein the at least one module (a), the at least one module (b), the at least one module (c), and the at least one compound (d) are linked to each other in any arrangement. In the above lists of preferred embodiments of modules (a), (b), and (c) and compound (d), respectively, an abbreviation is indicated for the specific module in brackets, which is used interchangeably with the full designation to refer to that specific module.

Preferably, a delivery system comprising or consisting of a conjugate for delivery of a compound into a cell according to the present invention comprises, essentially consists of, or consists of
(a) at least one module (a) that mediates cell targeting and facilitates cellular uptake, wherein the at least one module (a) is selected from the group consisting of a1, a2, a3, a4, a5, a6,a7, a8, a9, a10, a11, a12, a13, a14, a15, a16, a17, a18, a19, a20, a21, a22, a23, a24, a25, a26, a27, a28, a29, a30, a31, a32, a33, a34, a35, a36, a37, a38, a39, a40, a41, a42, a43, a44, a45, a46, a47, a48, a49, a50, a51, a52, a53, a54, a55, a56, a57, a58, a59, a60, a61, a62, a63, a64, a65, a66, a67, a68, a69, a70, a71, a72, a73, a74, a75, a76, a77, a78, a79, a80, a81, a82, a83, a84, a85, a86, a87, a88, a89, a90, a91, a92, a93, a94, a95, a96, a97, a98, a99, a100, a101, a102, a103, and a104,
(b) at least one module (b) that facilitates transport of modules (b) and (c) and compound (d) and, optionally module (a) to the endoplasmic reticulum (ER), wherein the at least one module (b) is selected from the group consisting of b1, b2, b3, b4, b5, b6, b7, b8, b9, b10, 11b, b12, b13, b14, b15, b16, b17, b18, b19, b20, b21, b22, b23, b24, and b25,
(c) at least one module (c) that mediates translocation of at least one compound (d) and, optionally one or more of the modules (a), (b) or (c) from the ER to the cytosol, wherein the at least one module (c) is selected from the group consisting of c1, c2, c3, c4, c5, c6,c7, c8, c9, c10, c11, c12, c13, c14, c15, c16, c17, c18, c19, c20, c21, c22, c23, c24, c25, c26, c27, c28, c29, c30, c31, c32, c33, c34, c35, c36, c37, c38, c39, c40, c41, c42, c43, c44, c45, c46, c47, c48, c49, c50, c51, c52, c53, c54, c55, c56, c57, c58, c59, c60, c61, c62, c63, c64, c65, c66, c67, c68, c69, c70, c71, c72, c73, c74, c75, c76, c77, c78, c79, c80, c81, c82, c83, c84, c85, c86, c87, c88, c89, c90, c91, c92, c93, c94, c95, c96, c97, c98, c99, c100, c101, c102, c103, c104, c105, c106, c107, c108, c109, c110, c111, c112, c113, c114, c115, c116, c117, c118, c119, c120, c121, c122, c123, c124, c125, c126, c127, c128, c129, c130, c131, c132, c133, c134, c135, c136, c137, c138, c139, c140, c141, c142, c143, c144, c145, c146, c147, c148, c149, c150, c151, c152, c153, c154, c155, c156, c157, c158, c159, c160, c161, c162, c163, c164, c165, c166, c167, c168, c169, c170, c171, c172, c173, c174, c175, c176, c177, c178, c179, c180, c181, c182, c183, c184, c185, c186, c187, c188, c189, c190, c191, c192, c193, c194, c195, c196, c197, c198, c199, c200, c201, c202, c203, c204, c205, c206, c207, c208, c209, 210, and c211, and
(d) at least one compound (d), wherein the at least one compound (d) is selected from the group consisting of d1, d2, d3, d4, d5, d6, d7, d8, d9, d10, d11, d12, d13, d14, d15, d16, d17, d18, d19, d20, d21, d22, d23, d24, d25, d26, d27, d28, d29, d30, d31, d32, d33, d34, d35, d36, d37, d38, d39, d40, d41, d42, d43, d44, d45, d46, d47, d48, d49, d50, d51, d52, d53, d54, d55, d56, d57, d58, d59, d60, d61, d62, d63, d64, d65, d66, d67, d68, d69, d70, d71, d72, d73, d74, d75, d76, d77, d78, d79, d80, d81, d82, d83, d84, d85, d86, d87, d88, d89, d90, d91, d92, d93, d94, d95, d96, d97, d98, d99, d100, d101, d102, d103, d104, d105, d106, d107, d108, d109, d110, d111, d112, d113, d114, d115, d116, d117, d118, d119, d120, d121, d122, d123, d124, d125, d126, d127, d128, d129, d130, d131, d132, d133, d134, d135, d136, d137, d138, d139, d140, d141, d142, d143, d144, d145, d146, d147, d148, d149, d150, d151, d152, d153, d154, d155, d156, d157, d158, d159, d160, d161, d162, d163, d164, d165, d166, d167, d168, d169, and d170,
   wherein the at least one module (a), the at least one module (b), the at least one module (c), and the at least one compound (d) are linked to each other in any arrangement.

In a preferred embodiment, the delivery system of the present invention further comprises a nuclear localization signal.

Preferably, the delivery system according to the first aspect of the invention comprises, essentially consists or consists of a conjugate of the second aspect of the invention.

The conjugate comprised in the delivery system according to the present invention comprises, essentially consists of or consists of at least one module (a), at least one module (b), at least one module (c) and at least one compound (d). The at least one module (a), the at least one module (b), the at least one module (c) and the at least one compound (d) of the conjugate of the present invention are linked to each other in any arrangement, combination, or stoichiometry.

It is noted that in those aspects of the first aspect, wherein the identical molecule is indicated as a preferred component for both module (a) and module (b), or module (a) and module (c), or modules (a), (b) and (c) it is preferred that this molecule is comprised only once in the conjugate comprised in the delivery system of the invention. Specific examples of such molecules, wherein a protein or, preferably a protein comprising several subunits not linked by peptide bonds, e.g. reduced toxicity of non-toxic variant of an AB-type or AB₅-type toxin, fulfills both the role of module (a) and (c) or (a), (b) and (c) are provided below as a second aspect of this invention, which, thus, may also be viewed as a preferred embodiment of the first aspect of the invention.

In a second aspect, the present invention relates to a delivery system for delivery of a compound into a cell comprising or consisting of at least one conjugate comprising, essentially consisting of or consisting of:
(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
   wherein at least two of the at least one module (a), the at least one module (b), and the at least one module (c) are comprised or contained within a multi-module protein or peptide, and wherein the multi-module protein or peptide, any remaining at least one module (a), at least one module (b), and at least one module (c) that are not comprised or contained within the multi-module protein or peptide, and the at least one compound (d) are linked to each other in any arrangement. The conjugates of the present invention optionally comprise a nuclear localization signal.

Thus, in this embodiment, the conjugate comprises or contains two or more of the modules of the within a single protein or peptide, i.e., a protein or peptide that comprises a cell targeting/uptake functionality [module (a)] and an ER transport functionality [module (b)], hereinafter defined as a [module (a)+module (b)] protein or peptide, a protein or peptide that comprises a cell targeting/uptake functionality [module (a)] and an ER to the cytosol translocation functionality [module (c)], hereinafter defined as a [module (a)+module (c)] protein or peptide, a protein or peptide that comprises an ER transport functionality [module (b)] and an ER to the cytosol translocation functionality [module (c)], hereinafter defined as a [module (b)+module (c)]protein or peptide, or a protein or peptide that comprises a cell targeting/uptake functionality [module (a)], an ER transport functionality [module (b)], and an ER to the cytosol translocation functionality [module (c)], hereinafter defined as a [module (a)+module (b)+module (c)] protein or peptide. Within these embodiments, the two or more modules may be linked to each other as a contiguous protein or peptide or may be provided by different domains or subunits of a protein or peptide which are preferably linked via disulfide bonds formed between Cys-residues in each of the two or more protein chains forming the protein, and may be linked to or associated with each other in any arrangement, combination, or stoichiometry. Preferred examples of such proteins, which are present in different domains, are AB-type or AB₅-type holotoxins, which are known from plants and bacteria. Various examples of such holotoxins are provided herein. The AB-type holotoxins comprise one subunit chain of type A and one subunit chain of type B, which are preferably not linked by peptide bonds but rather by disulfide bonds. The AB₅-type holotoxins comprise one A-type chain subunit and five B-type chain subunits, which are preferably not linked by peptide bonds but by disulfide bonds.

### Preferred Arrangements of the Modules in the Various Aspects of the Invention

Unless it is specifically indicated above, that two or more modules are linked in a particular arrangement, e.g. if modules (b) and (c) form a contiguous peptide or protein and thus, the relative linkage of the two or more modules is predetermined, the modules may be linked in any of the following arrangements. Preferably, the modules (a), (b), and (c) and the compound (d) of the conjugate of the present invention are linked to each other in one of the following arrangements or combinations: (a), (b), (c) and (d); (b), (a), (c) and (d); (b), (c), (a) and (d); (c), (b), (a) and (d); (a), (c), (b) and (d); (c), (a), (b) and (d); (c), (d), (b) and (a); (d), (c), (b) and (a); (b), (d), (c) and (a); (d), (b), (c) and (a); (b), (c), (d) and (a); (c), (b), (d) and (a); (c), (d), (a) and (b); (d), (c), (a) and (b); (a), (d), (c) and (b); (d), (a), (c) and (b); (a), (c), (d) and (b); (c), (a), (d) and (b); (b), (d), (a) and (c); (d), (b), (a) and (c); (a), (d), (b) and (c); (d), (a), (b) and (c); (a), (b), (d) and (c); or (b), (a), (d) and (c), wherein in each arrangement or combination at least one module (a), at least one module (b), at least one module (c) and at least one compound (d) is present. The respectively indicated order of the modules (a), (b) and (c) and the compound (d) signifies the links between the modules and compound, respectively. Thus, in the arrangement (a), (b), (c) and (d), (a) is linked to (b), (b) is linked to (c) and (c) is linked to (d).

The term “linked” in this context has the meaning as defined above and as more specifically taught below, e.g. includes covalent linkages, non-covalent linkages and linkages via linker molecules.

It is particularly preferred that the modules (a), (b), and (c) and the compound (d) of the conjugate of the present invention are linked to each other in one of the following arrangements or combinations: (a)ₓ, (b)_{y}, (c)_{z} and (d)ₙ; (b)_{y}, (a)ₓ, (c)_{z} and (d)ₙ; (b)_{y}, (c)_{z}, (a)ₓ and (d)ₙ; (c)_{z}, (b)_{y}, (a)ₓ and (d)ₙ; (a)ₓ, (c)_{z}, (b)_{y} and (d)ₙ; (c)_{z}, (a)ₓ, (b)_{y} and (d)ₙ; (c)_{z}, (d)ₙ, (b)_{y} and (a)ₓ; (d)ₙ, (c)_{z}, (b)_{y} and (a)ₓ; (b)_{y}, (d)ₙ, (c)_{z} and (a)ₓ; (d)ₙ, (b)_{y}, (c)_{z} and (a)ₓ; (b)_{y}, (c)_{z}, (d)ₙ and (a)ₓ; (c)_{z}, (b)_{y}, (d)ₙ and (a)ₓ; (c)_{z}, (d)ₙ, (a)ₓ and (b)_{y}; (d)ₙ, (c)_{z}, (a)ₓ and (b)_{y}; (a)ₓ, (d)ₙ, (c)_{z} and (b)_{y}; (d)ₙ, (a)ₓ, (c)_{z} and (b)_{y}; (a)ₓ, (c)_{z}, (d)ₙ and (b)_{y}; (c)_{z}, (a)ₓ, (d)ₙ and (b)_{y}; (b)_{y}, (d)ₙ, (a)ₓ and (c)_{z}; (d)ₙ, (b)_{y}, (a)ₓ and (c)_{z}; (a)ₓ, (d)ₙ, (b)_{y} and (c)_{z}; (d)ₙ, (a)ₓ, (b)_{y} and (c)_{z}; (a)ₓ, (b)_{y}, (d)ₙ and (c)_{z}; or (b)_{y}, (a)ₓ, (d)ₙ and (c)_{z}, wherein x is an integer of 1 to 5, i.e. 1, 2, 3, 4, or 5, preferably of 1; y is an integer of 1 to 5, i.e. 1, 2, 3, 4, or 5, preferably of 1; z is an integer of 1 to 5, i.e. 1, 2, 3, 4, or 5, preferably of 1; and n is an integer of 1 to 50, i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, preferably of 2, 3, 4, 5, 6, 7, 8, 9, or 10, more preferably of 2, 3, 4, or 5.

A conjugate according to the present invention that comprises more than one compound (d) can deliver more compounds (d) into a cell, thus the efficiency of delivering a compound (d) can be increased compared to a conjugate according to the present invention that comprises modules (a), (b) and (c) and only one compound (d). Preferably, the conjugate according to the present invention comprises at least 2-50 compounds (d). More preferably, the conjugate according to the present invention comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 compounds (d). More preferably, the conjugate according to the present invention comprises at least 2, 3, 4, or 5 compounds (d). Preferably, the conjugate comprising more than one compound (d) comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 compounds (d) that are the same or different.

In a preferred embodiment, the conjugate comprising more than one compound (d) comprises at least 2 of the same compounds (d). Preferably, the at least 2 of the same compounds (d) are selected from the group consisting of 2 nucleic acids, 2 proteins, 2 peptides, 2 antigens, 2 enzymes, 2 small molecules, 2 therapeutic molecules, 2 diagnostic molecules, and 2 imaging molecules. Preferably, the at least 2 same compounds (d) comprise at least 2 of the same nucleic acids. More preferably, the at least 2 same compounds (d) comprise at least 2 of the same siRNAs.

In another preferred embodiment, the conjugate comprising more than one compound (d) comprises at least 2 different compounds (d). Preferably, the at least 2 different compounds (d) comprise a first compound (d) selected from the group consisting of a nucleic acid, a protein, a peptide, an antigen, an enzyme, a small molecule, a therapeutic molecule, a diagnostic molecule, and an imaging molecule; and a second compound (d) selected from the group consisting of a nucleic acid, a protein, a peptide, an antigen, an enzyme, a small molecule, a therapeutic molecule, a diagnostic molecule, and an imaging molecule, wherein the first compound (d) and the second compound (d) are different from each other. In a preferred embodiment, the at least 2 different compounds (d) comprise at least 2 different nucleic acids. Preferably, the at least 2 different compounds (d) comprise at least 2 different siRNAs directed to the same target. In another preferred embodiment, the at least 2 different compounds (d) comprise at least 2 different siRNAs directed to at least 2 different targets. In another preferred embodiment, the at least 2 different compounds (d) comprise at least one nucleic acid and at least one protein or peptide. Preferably, the at least one nucleic acid is an siRNA and the at least one protein or peptide is a RISC protein or peptide.

Conjugates of the present invention, wherein the module (b) or the modules (b) are positioned within the arrangement in a way that they are linked to only one other module or compound are preferred to avoid or to at least minimize steric hindrance by the other modules and/or compound(s) of the conjugate or other undesired interactions. Thus, preferred embodiments of the conjugate of the present invention are (c), (d), (a) and (b); (d), (c), (a) and (b); (a), (d), (c) and (b); (d), (a), (c) and (b); (a), (c), (d) and (b); and (c), (a), (d) and (b), wherein in each embodiment at least one module (a), at least one module (b) and at least one module (c) and at least one compound (d) is present. The presence of module (b) in the indicated position has the advantage that module (b) is free and unhindered by the other modules (a) and (c) and by compound (d) so that steric hindrance or other undesired interactions can be avoided or at least minimized. If module (b) comprises, essentially consists or consists of an oligopeptide, it is preferred that the C-terminus of such oligopeptide is free and that any linkage, be it covalent or non-covalent, to further modules, compound(s) or linker molecule occurs at or close to the N-terminus of such oligopeptide.

Particularly preferred embodiments of the conjugate of the present invention are the following arrangements (c)_{z}, (d)ₙ, (a)ₓ and (b)_{y}; (d)ₙ, (c)_{z}, (a)ₓ and (b)_{y}; (a)ₓ, (d)ₙ, (c)_{z} and (b)_{y}; (d)ₙ, (a), (c)_{z} and (b)_{y}; (a)ₓ, (c)_{z}, (d)ₙ and (b)_{y}; and (c)_{z}, (a)ₓ, (d)ₙ and (b)_{y}, wherein x is an integer of 1 to 5, i.e. 1, 2, 3, 4, or 5, preferably of 1; y is an integer of 1 to 5, i.e. 1, 2, 3, 4, or 5, preferably of 1; z is an integer of 1 to 5, i.e. 1, 2, 3, 4, or 5; preferably of 1; and n is an integer of 1 to 10, i.e. 1, 2, 3, 4, 5, 6, 7, 8, 9 or 10, preferably of 3. Accordingly, it is particularly preferred that x is 1, y is 1, z is 1 and n is an integer of 1 to 50, i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, preferably of 2, 3, 4, 5, 6, 7, 8, 9, or 10, more preferably of 2, 3, 4, or 5.

Conjugates of the present invention, wherein compound (d) or compounds (d) are positioned in second position or third position and module (b) or modules (b) are positioned within the arrangement in a way that they are linked to only one other module or compound, e.g. positioned in last position of the arrangement, i.e., wherein the C-terminus of module (b) or modules (b) is free, are preferred. Therefore, particularly preferred embodiments of the conjugate of the present invention are (c), (d), (a) and (b); (a), (d), (c) and (b); (a), (c), (d) and (b); and (c), (a), (d) and (b), wherein in each embodiment at least one module (a), at least one module (b), at least one module (c) and at least one compound (d) is present. The presence of compound (d) in second or third position has the advantage that the entrance of compound (d) into the cell and further within the cell is facilitated by avoiding steric hindrance by compound (d) for the biological action of modules (a), (b) and (c). In addition, module (b) is free and unhindered by the other modules (a) and (c) and by compound (d) so that steric hindrance and other undesired interactions can be avoided or at least minimized.

Particularly preferred embodiments of the conjugate of the present invention are (c)_{z}, (d)ₙ, (a)ₓ and (b)_{y}; (a)ₓ, (d)ₙ, (c)_{z} and (b)_{y}; (a)ₓ, (c)_{z}, (d)ₙ and (b)_{y}; and (c), (a)ₓ, (d)ₙ and (b)_{y}, wherein x is an integer of 1 to 5, i.e. 1, 2, 3, 4, or 5, preferably of 1; y is an integer of 1 to 5, i.e. 1, 2, 3, 4, or 5, preferably of 1; z is an integer of 1 to 5, i.e. 1, 2, 3, 4, or 5; preferably of 1; and n is an integer of 1 to 50, i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, preferably of 2, 3, 4, 5, 6, 7, 8, 9, or 10, more preferably of 2, 3, 4, or 5. Accordingly, it is particularly preferred that x is 1, y is 1, z is 1 and n is an integer of 1 to 50, i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, preferably of 2, 3, 4, 5, 6, 7, 8, 9, or 10, more preferably of 2, 3, 4, or 5.

In the most preferred embodiments of the conjugate of the present invention, wherein module (b) is arranged terminally, preferably in last position, wherein its C-terminus is free, and compound (d) in second or third position, the arrangements of the modules (a), (b) and (c) and of the compound (d) and the number of the modules (a), (b) and (c) and of the compound (d) are as follows:
(i) (a)ₓ, (c)_{z}, (d)ₙ, and (b)_{y}, wherein x is an integer of 1, z is an integer of 1, n is an integer of 1 and y is an integer of 1,
(ii) (a)ₓ, (c)_{z}, (d)ₙ, and (b)_{y}, wherein x is an integer of 1, z is an integer of 1, n is an integer of 2 and y is an integer of 1,
(iii) (a)ₓ, (c)_{z}, (d)ₙ, and (b)_{y}, wherein x is an integer of 1, z is an integer of 1, n is an integer of 3 and y is an integer of 1,
(iv) (a)ₓ, (d)ₙ, (c)_{z} and (b)_{y}, wherein x is an integer of 1, n is an integer of 1, z is an integer of 1 and y is an integer of 1,
(v) (a)ₓ, (d)ₙ, (c)_{z} and (b)_{y}, wherein x is an integer of 1, n is an integer of 2, z is an integer of 1 and y is an integer of 1, or
(vi) (a)ₓ, (d)ₙ, (c)_{z} and (b)_{y}, wherein x is an integer of 1, n is an integer of 3, z is an integer of 1 and y is an integer of 1.

Preferably, the at least one module (a), the at least one module (b), the at least one module (c) and the at least one compound (d) of the conjugate of the present invention, which are arranged to each other in any order, combination, or stoichiometry, are linked to each other via a covalent linkage, are linked to each other via a non-covalent linkage, are linked to each other via at least one adapter molecule and/or are linked to each other via at least one linker molecule that optionally comprises at least one adapter molecule.

The term “covalent linkage” means a type of chemical linkage, wherein each atom of a bond pair contributes one electron to form a pair of electrons in a chemical bond.

The term “non-covalent linkage” means a type of chemical linkage, typically between macromolecules, that does not involve the sharing of pairs of electrons, but rather involves more dispersed variations of electromagnetic interactions.

The term “linker molecule” in the context of the present invention refers to a molecule that is able to attach or conjugate two molecules or compounds to each other. This attachment or conjugation can be achieved via a covalent linkage. Thus, any molecule having the above mentioned characteristics can be used to link the modules and the compound of the conjugate of the present invention to each other. Preferably, the linker molecule serves the purpose of spatially separating the various modules and the compound(s) to avoid steric hindrance between the modules and the compound. Such steric hindrance may inhibit access and/or interaction with the cellular structures, e.g. proteins, lipids or carbohydrate chains, to which the modules have to bind or to interact; to exert their respective function as outlined herein. Linker molecules may also be used within the conjugates of the invention to covalently modify the terminus of an siRNA to enable its covalent connection to an aminooxyacetyl comprising delivery vehicle or conjugate.

The term “adapter molecule” in the context of the present invention refers to a molecule that forms an indirect and non-covalent linkage, e.g. between a module [e.g. module (a)] and a compound (d). For example, the adapter molecule, wherein it is covalently linked to module (a), can be used to indirectly and non-covalently link module (a) to compound (d), wherein the adaptor molecule forms a non-covalent linkage to compound (d). As such, the adapter molecule also functions as a spacer to keep the compound (d) at a distance from the module (a). The indirect and non-covalent linkage is based on ionic (electrostatic) interactions or hydrophobic interactions.

The different types of linkages are exemplified in the following description for the conjugation of module (a) to compound (d). It shall be understood that this exemplification is applicable to any module-module, any module-compound (d), or any compound (d)-compound (d) conjugation. For example, module (a) of the conjugate of the present invention can be directly linked to compound (d) via a non-covalent linkage. Module (a) of the conjugate of the present invention can also be directly linked to compound (d) via a covalent linkage. Module (a) of the conjugate of the present invention can further be linked indirectly and covalently to compound (d) via a linker molecule, which forms a covalent linkage with module (a) and with compound (d). In addition, compound (d) can be linked indirectly to module (a) via an adapter molecule, wherein the adapter molecule and compound (d) are connected to each other via a non-covalent linkage and the adapter molecule is covalently linked to module (a). Further, compound (d) can be indirectly linked to module (a) via an adapter molecule and a linker molecule, wherein the adapter molecule and compound (d) are connected to each other via a non-covalent linkage, and the adapter molecule is covalently linked to a linker molecule which links module (a) and an adjacent module [e.g. module (c) or (b)].

The modules and the compound of the conjugate of the present invention can be linked via different linkage types to each other. Thus, the conjugate of the present invention does not necessarily comprise modules and a compound linked to each other via the same linkage type. For example, covalent linkages can be used with non-covalent linkages and/or with covalent linkages via linker molecules or adapter molecules. Depending upon the desired target cell delivery strategy, the conjugate can be designed with specific covalent and/or non-covalent linkages, with or without an adapter molecule and/or linker molecule. In this way, one of ordinary skill in the art can make different types of conjugates that are useful for different applications.

Preferably, the at least one module (a), the at least one module (b), the at least one module (c) and the at least one compound (d) of the conjugate according to the present invention are covalently linked to each other, preferably via a disulfide-linkage, an amide-linkage, an oxime-linkage and/or a hydrazone-linkage.

The term “disulfide-linkage” (disulfide-bond) refers to a chemical bond, which is usually derived by the coupling of two thiol groups. The linkage is also called an SS-bond or disulfide bridge. Disulfide bonds in proteins are formed between the thiol groups of cysteine residues.

The term “amide-linkage” (peptide bond) refers to a chemical bond formed between two proteins or peptides when the carboxyl group of one molecule reacts with the amine group of the other molecule, thereby releasing a molecule of water (H₂O).

The term “oxime-linkage” refers to a chemical bond, which is derived by coupling of a protein or peptide carrying aglyoxylic aldehyde functionality to a protein or peptide functionalized with an aminooxy group. The oxime linkage is obtained by reaction of an aldehyde or ketone with a hydroxylamine or aminooxy modified component. It can be used to link together all manner of molecules, i.e. small molecules, sugars, peptides, proteins, oligonucleotides, etc. These functionalities may be present in a synthesized component of a conjugate of the invention, or one or both of the functionalities may be introduced into a component of a conjugate of the invention. In a preferred method of preparing a conjugate of the present invention, an aminooxy modification is included in a synthetic peptide and a benzaldehyde function is attached to an siRNA.

The term “hydrazone-linkage” (hydrazone-bond) refers to a chemical bond, which is derived by condensing proteins or peptides with each other that are modified at their amino groups to contain an average of three to six aryl aldehyde or acyl hydrazide groups. The hydrazone linkage is obtained by reaction of an aldehyde or ketone with a hydrazine or acylhydrazine modified component. An “acylhydrazone linkage” is obtained by reaction of an aldehyde or ketone with an acylhydrazine modified component. Commercial reagent kits are available and may be used within the methods of the present invention to couple or connect two biomolecules of use in a conjugate of the present invention.

There are four commonly known types of non-covalent interactions: hydrogen bonds, ionic bonds, Van der Waals forces, and hydrophobic interactions, which may be the basis for the interaction of the modules and/or compound(s) used in the conjugates of the present invention.

Preferably, the at least one module (a), the at least one module (b), the at least one module (c) and/or the at least one compound (d) of the conjugate according to the present invention are linked to each other via non-covalent linkage, preferably an ionic (electrostatic) linkage and/or via a hydrophobic linkage.

The term “hydrophobic interaction” (hydrophobic linkage) refers to an interaction dependent from the tendency of hydrocarbons (or of lipophilic hydrocarbon-like groups in solutes) to form intermolecular aggregates in an aqueous medium.

The term “ionic (electrostatic) linkage” (ionic bond or electrostatic bond) refers to a non-covalent bond in which one atom loses an electron to form a positive ion and the other atom gains to electron to form a negative ion. In biological systems, most electrostatic bonds or interactions are between groups that are protonated and others that are deprotonated, i.e., a lysine or arginine side chain amino group interacting with either a carboxylate group of a protein or a phosphate group in a DNA or RNA molecule.

A particularly preferred linker molecule according to the present invention is a protein, a peptide, a modified peptide, an amino acid residue, a modified amino acid residue or a hydrophilic carbohydrate chain, preferably a polydiol chain with between 1 to 20 repeat units, i.e. 1, 2, 3, 4, 5, 6, 7, 8, 9, 10, 11, 12, 13, 14, 15, 16, 17, 18, 19 or 20, preferably polyethylene glycol (PEG), wherein between 1 to 20, i.e. 1, 2, 3, 4, 5, 6, 7, 8, 9, 10, 11, 12, 13, 14, 15, 16, 17, 18, 19 or 20, ethyleneglycol units are connected to each other. These linker molecules link the at least one module (a), the at least one module (b), the at least one module (c) and/or the at least one compound (d) to each other via a covalent linkage, preferably via an amide-linkage or a disulfide-linkage.

Said linker molecules can also be combined with each other, e.g. a peptide linker can be combined with a modified amino acid residue linker, or a modified amino acid residue linker can be combined with a modified peptide linker to covalently link 1) at least one module (a) to at least one module (b) or at least one module (c); 2) at least one module (b) to at least one module (a) or at least one module (c); 3) at least one module (a) to at least one module (b) and at least one module (c); or 4) at least one module (a), at least one module (b), and/or at least one module (c) to at least one compound (d). Preferably, the at least one module (a), the at least one module (b), or the at least one module (c) are covalently linked via an amide linkage. Preferably, the at least one module (a), the at least one module (b), and/or the at least one module (c) are/is covalently linked to the at least one compound (d) via a disulfide linkage.

The term “peptide linker” according to the present invention means a chain of amino acid residues which may be naturally occurring or derivatives of naturally occurring amino acid residues and which are preferably linked via peptide or disulfide bonds.

Preferably, the peptide linker of the present invention consists of between 2 and 50 or between 2 and 30 amino acid residues or amino acid residue derivatives, preferably of between 2 and 20 or between 2 and 15 amino acid residues or amino acid residue derivatives, and more preferably of between 2 and 10, between 2 and 5, or 2, 3, 4, 5, 6, 7, 8, 9 or 10 amino acid residues or amino acid residue derivatives. Preferably, the linker sequence is flexible so as not to hold the conjugate in a single rigid conformation. The peptide linker can be used to space the modules (a), (b) and (c) from each other and/or to space the modules (a), (b) and (c) from the compound (d). For example, two peptide linkers can be positioned in a conjugate of the present invention having the precise arrangement: module (a), a first peptide linker, compound (d), a second peptide linker, module (c) and module (b), such that a first peptide linker is positioned between module (a) and compound (d) and a second peptide linker is positioned between compound (d) and module (c), to provide molecular flexibility of and/or around compound (d). One of ordinary skill in the art can position the peptide linker or peptide linkers within the conjugate as necessary and specific to the modules, compound and intended use of the conjugate, and without undue experimentation. The length of the peptide linker is chosen to optimize the biological activity of the conjugate comprising the compound and can be determined empirically without undue experimentation. The linker peptide should be long enough and flexible enough to allow unhindered functionality of the modules and of the compound and to avoid steric or other undesired interactions. Examples of peptide linkers include but are not limited to GGGGS (SEQ ID NO: 92), GKSSGSGSESKS (SEQ ID NO: 93), GSTSGSGKSSEGKG (SEQ ID NO: 94), GSTSGSGKSSEGSGSTKG (SEQ ID NO: 95), GSTSGSGKPGSGEG STKG (SEQ ID NO: 96), EGKSSGSGSESKEF (SEQ ID NO: 97), and SGSGSG [(SG)₃; SEQ ID NO: 98]. Other suitable linker peptides are those as previously described in the literature [18-20] and in U.S. Pat. No. 4,751,180, U.S. Pat. No. 4,935,233, and the like.

In a particularly preferred embodiment, a peptide linker for use in a conjugate of the present invention comprises a degron peptide. A degron peptide may be used in the linker peptide of the conjugates of the present invention to link the at least one compound (d) to the conjugate, preferably in lieu of a disulfide bridge, and to target degradation of the delivery vehicle while delivering the compound (d) to the cell cytoplasm. Preferably, the degron peptide comprises, consists essentially of, consists of, or contains a degron based on the F protein derived from the HCV-1 isolate (genotype 1a; http://www.uniprot.org/uniprot/P0C045; see Yuksek et al., J. Virol. 2009 83(2):612-21. Epub 2008 Oct. 29), a degron peptide comprising HRTSSSRVAVRSLVEFT CCRAGALDWVCARRGRLPSGRNLE (SEQ ID NO: 99), a degron peptide comprising MPVAGSELPRRPLPPAAQERDAEPRPPHGELQYLGQIQHILRCGV (SEQ ID NO: 100, from human thymidylate synthase; see Pena et al., J Biol. Chem. 2009, 284(46):31597-607. Epub 2009 Sep. 21), a degron peptide comprising FPPEVEEQDDGTLPMSCAQES GMDRHPAACASARINV (SEQ ID NO: 101; from mouse ornithine decarboxylase; see Takeuchi et al., Biochem J. 2008, 410:401-407), a degron peptide comprising PTSPDRPGSTSPFAPSATDLPSMPEPALTSR (SEQ ID NO: 102; see Bhat et al., J. Biol. Chem. 2010, 285:25893-25903), or a degron peptide comprising EDEDSDWDSVSNDSEFY ADEDDEEYDDYNEEEAD (SEQ ID NO: 103; from yeast Mks1P; see Liu et al., 2005. Mol. Biol. Cell 16:4893-4904).

The term “modified peptide linker” according to the present invention means a chain of amino acid residues that may be naturally occurring or a derivative of naturally occurring amino acid residues, preferably linked via peptide bonds, which are further chemically modified. A preferred modified peptide linker is a peptide covalently bound to polyethyleneglycol (PEG). Such a modified peptide linker can be predominantly composed of short polyethylenglycol (PEG) repeats that facilitate its synthesis. PEG is already approved for delivery and stabilization of peptide based therapeutics and is non-toxic. For example, N-Fmoc-amido-dPEG₁₂-acid can be utilized as a spacer to replace a repeat of several amino acid residues to simplify the synthesis, improve solubility, and ensure flexibility of the linker that connects the various functional domains within the synthetic peptide.

The term “amino acid residue linker” encompasses naturally occurring amino acids as well as amino acid derivatives. Preferably, the amino acids of the amino acid linker are small amino acids or hydrophobic non-aromatic amino acids. A small amino acid in the context of the present invention is preferably an amino acid having a molecular weight of less than 125 Dalton. Preferably, a small amino acid is selected from the group consisting of the amino acids glycine, alanine, serine, cysteine, threonine, valine, and derivatives thereof. A hydrophobic non-aromatic amino acid in the context of the present invention is preferably any amino acid which has a Kyte-Doolittle hydropathy index of higher than 0.5, more preferably of higher than 1.0, even more preferably of higher than 1.5 and is not aromatic. Preferably, a hydrophobic non-aromatic amino acid in the context of the present invention, is selected from the group consisting of the amino acids alanine (Kyte Doolittle hydropathy index 1.8), methionine (Kyte Doolittle hydropathy index 1.9), isoleucine (Kyte Doolittle hydropathy index 4.5), leucine (Kyte Doolittle hydropathy index 3.8), valine (Kyte Doolittle hydropathy index 4.2), and derivatives thereof having a Kyte Doolittle hydropathy index as defined above.

The term “modified amino acid residue linker” encompasses naturally occurring amino acids as well as amino acid derivatives that are chemically modified. For example, modified amino acids are prepared by reacting single amino acids with an acylating or sulfonating agent that reacts with free amino moieties present in the amino acids to form amides or sulfonamides, respectively. A preferred modified amino acid linker is an amino acid that is acetylated or sulfonated. Also preferred is the use of activated cysteine [C(NPyS)] as a modified amino acid linker.

In another embodiment, a conjugate of the present invention comprises a “toxin-based linker”, wherein the toxin-based linker comprises a toxin A2-subunit or a non-toxic or reduced toxicity toxin A1-subunit. Preferably, toxin-based linkers are used to link a toxin B subunit to any remaining module(s) and/or compound (d) of the conjugate. Thus, these toxin-based linkers, e.g., a toxin A2-subunit or non-toxic or reduced toxicity toxin A1 subunit protein or peptide, provide a natural linker for use in the conjugates according to the invention. In a preferred embodiment, a conjugate of the present invention comprises a toxin A2 subunit peptide linker comprising, consisting essentially, or consists of acetyl-(L-propargylglycyl)-MASDEFPS MSPADGRVRGITHNKILWDSSTLGAILMRRTISS (SEQ ID NO: 308; an Stx1b Shiga toxin A2 subunit-modified peptide linker in which the naturally occurring C at position 10 is replaced by the isosteric S to avoid problems with the cysteine thiol); acetyl-(L-propargylglycyl)-AVNEESQPESQITGDRPVIKINNTLWESNTAAAFLNRKSQFLYTTGK (SEQ ID NO: 309, an Stx2a Shiga toxin A2 subunit-modified peptide linker in which the naturally occurring C at position 10 is replaced by the isosteric S to avoid problems with the cysteine thiol); or acetyl-(L-propargylglycine)-MSNTSDEKTQSLGVKFLDEYQSKVKRQIFSGYQSDIDTHNRIKDEL (SEQ ID NO: 310, a cholera toxin A2 subunit-modified peptide linker).

An adapter molecule forms an indirect and non-covalent linkage, e.g. between a module [e.g. module (a), (b) or (c), preferably module (a)] and a compound (d), preferably via ionic (electrostatic) interactions or hydrophobic interactions.

In a preferred embodiment of a conjugate of the present invention, the adapter molecule indirectly and non-covalently links module (a) to compound (d) by forming a non-covalent linkage to compound (d), e.g. via hydrophobic interactions, wherein the adapter molecule is covalently linked to module (a). In addition, module (a) is covalently linked to module (c) and module (c) is covalently linked to module (b).

In another preferred embodiment of a conjugate of the present invention, an adapter molecule interacts with a compound (d) via an ionic (e.g., electrostatic) interaction or a hydrophobic interaction, wherein the adapter molecule is covalently linked to a linker molecule that connects a module (a) with a module (c). In addition, the module (c) is covalently linked to a module (b). As a result, the module (a) and the compound (d) are indirectly and non-covalently linked to each other via the adapter molecule. Thus, a conjugate of the present invention preferably comprises a linker molecule between module (a) and module (c), wherein the linker molecule is covalently linked to an adaptor molecule that is non-covalently linked to the compound (d). Preferably, the adapter molecule branches off from a side chain of the linker molecule.

Generally, one or more adapter molecules can be used to indirectly and non-covalently link, e.g. a compound (d) and a module, e.g. module (a), (b) or (c), preferably module (a), to each other. In a preferred embodiment of the conjugate of the present invention, 2, 3, 4, or 5 adapter molecules are used to indirectly and non-covalently link a compound (d) and a module, e.g. module (a), (b) or (c), preferably module (a), to each other. More preferably, 2 adapter molecules are used in the conjugate of the present invention to indirectly and non-covalently link a compound (d) and a module, e.g. module (a), (b) or (c), preferably module (a), to each other.

For example, in a preferred embodiment, a conjugate of the present invention comprises two (2) adapter molecules that each interact with a compound (d) via ionic (electrostatic) interactions and/or hydrophobic interactions, and wherein each of the two adapter molecules are covalently linked to a module (a) of the conjugate. In addition, the module (a) is covalently linked to a module (c), and the module (c) is covalently linked to a module (b). Thus, as a result, the module (a) and the compound (d) are indirectly and non-covalently linked to each other via the two adapter molecules. Preferably, the two adaptor molecules are the same. The resulting conjugate of this preferred embodiment of the invention has an increased ratio of compound (d) to delivery vehicle [i.e., modules (a), (b), and (c)].

Preferably, modules (b) and (c) are not used to covalent link to the adapter molecule to minimize the risk of interfering with their functionalities.

Preferred adapter molecules are nucleic acid binding domains of proteins such as RNA binding proteins or double stranded RNA (dsRNA) binding proteins (DRBPs), double stranded DNA (dsDNA) binding proteins (DDBPs), single chain antibodies or ligand binding domains of surface receptors. More preferred adapter molecules that may be used in the conjugates of the present invention to indirectly and non-covalently link or conjugate a module and a compound to each other are double stranded RNA binding proteins (DRBPs). The DRBP may be used in the present invention for different functions. It may function as a spacer to keep compound (d) at a distance from module(s) (a), (b), and/or (c). It may also form a stable indirect and non-covalent linkage between a compound (d) and a module, e.g. module (a), (b) or (c), preferably module (a). DRBP may also serve to neutralize or reduce the anionic charge of a compound (d) to be delivered using modules (a), (b) and (c). DRBP may further promote the uptake of a conjugate of the present invention by sufficiently reducing the anionic charge of a compound (d) such that the cationic charge of the modules (a), (b) and (c) is sufficient to enter the cell by an endocytic event.

The use of a DRBP adaptor(s) or a DDBP adaptor(s) is preferred when compound (d) is a nucleic acid. When compound (d) is a double stranded RNA (dsRNA), a conjugate of the present invention comprises a DRBP adaptor(s). When compound (d) is a double stranded DNA (dsDNA), a conjugate of the present invention comprises a DDBP adaptor(s).

Preferred dsRNA binding proteins (DRBPs) that can be employed as adapter molecules in the conjugates of the present invention and their Accession numbers in parenthesis include: PKR (AAA36409, AAA61926, Q03963), TRBP (P97473, AAA36765), PACT (AAC25672, AAA49947, NP_{—}609646), Staufen (AAD17531, AAF98119, AAD17529, P25159), NFAR1 (AF167569), NFAR2 (AF167570, AAF31446, AAC71052, AAA19960, AAA19961, AAG22859), SPNR (AAK20832, AAF59924, A57284), RHA (CAA71668, AAC05725, AAF57297), NREBP (AAK07692, AAF23120, AAF54409, T33856), kanadaptin (AAK29177, AAB88191, AAF55582, NP_{—}499172, NP_{—}198700, BAB19354), HYLL (NP_{—}563850), hyponastic leaves (CAC05659, BAB00641), ADAR1 (AAB97118, P55266, AAK16102, AAB51687, AF051275), ADAR2P78563, P51400, AAK17102, AAF63702), ADAR3 (AAF78094, AAB41862, AAF76894), TENR (XP059592, CAA59168), RNaseIII (AAF80558, AAF59169, Z81070Q02555/S55784, P05797), and Dicer (BAA78691, AF408-401, AAF56056, 544849, AAF03534, Q9884), RDE-4 (AY071926), FLJ20399 (NP_{—}060273, BAB26260), CG1434 (AAF48360, EAA12065, CAA21662), CG13139 (XP059208, XP143416, XP110450, AAF52926, EEA14824), DGCRK6 (BAB83032, XP110167) CG1800 (AAF57175, EAA08039), FLJ20036 (AAH22270, XP134159), MRP-L45 (BAB14234, XP129893), CG2109 (AAF52025), CG12493 (NP_{—}647927), CG10630 (AAF50777), CG17686 (AAD50502), T22A3.5 (CAB03384) and accession number EAA14308. The sequences of such DRBPs are known in the art and can be obtained via their corresponding accession numbers.

A DRBP sequence for use in the present invention is FFMEELNTYRQKQGVVLKYQELP NSGPPHDRRFTFQVIIDGREFPEGEGRSKKEAKNAAAKLAVEILNKE (SEQ ID NO: 104; see also [21-22]). This preferred DRBP sequence is a dsRNA binding domain (DRBD) sequence, rather than a full DRBP sequence and is derived by truncation from PKR (Accession numbers AAA36409, AAA61926, Q03963).

More preferred adaptor molecules are variants of wild-type double stranded RNA binding proteins (DRBP variants) that have a reduced ability to bind dsRNA than the respective naturally occurring DRBPs mentioned above and are, therefore, less likely to interfere with the intended biological activity of the compound in the cell.

A DRBP variant which is more preferred in the present invention differs from the DRBP protein from which it is derived by up to 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145 or 150 amino acid changes in the amino acid sequence (i.e., substitutions, insertions, deletions, N-terminal truncations and/or C-terminal truncations). The amino acid substitutions may be conservative or non-conservative. A DRBP variant, which is preferred in the present invention can alternatively or additionally be characterised by a certain degree of sequence identity to the DRBP protein from which it is derived. Thus, the DRBP variants, which are preferred in the present invention have a sequence identity of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to the respective reference (i.e., wild-type) DRBP.

Additionally, a DRBP variant is only regarded as a DRBP variant within the context of the present invention, if it exhibits the relevant biological activity to a degree of at least 30% of the activity of the wild-type DRBP protein. The relevant “biological activity” in the context of the present invention is the “binding activity”, i.e. the ability of the DRBP variant to bind the compound. One of ordinary skill in the art can readily assess whether a DRBP variant has a reduced dsRNA binding activity, i.e. at least 30% of the activity of the wild-type DRBP protein. Suitable assays, e.g. binding assays, for determining the “binding activity” of the DRBP variant compared to the binding activity of the wild-type DRBP are known to the person of ordinary skill in the art [22, 23].

Preferred dsDNA binding proteins (DDBPs) that can be employed as adapter molecules in the conjugates of the present invention are any protein or protein domain that comprising one of the following known DNA binding motifs: a helix-turn-helix motif, a zinc finger motif, a leucine zipper motif, a winged helix (turn helix) motif, a helix-loop-helix motif, or an HMG-box motif In a particular embodiment, a conjugate of the present invention comprises a DDBP selected from the group consisting of HMGB1/2 (high-mobility group box 1 and 2 proteins, GeneIDs: 3146 and 3148, respectively), crp (GeneID 947867), Egr1 (GeneID 1958), Jun (GeneID 3725), FOXA1(forkhead box A1; GeneID 3169), ETS1 (GeneID 2113), Twist1 (GeneID 22160), HIST2H2AC (histone cluster 2, GeneID 8338), and the like.

It is particularly preferred that the modules (a), (b), (c) and the compound (d) of the conjugate of the present invention have the following arrangements or combinations and comprise the following linkage types:
(i) (a)ₓ, (c)_{z}, (d)ₙ and (b)_{y}, wherein (a)ₓ is covalently linked to (c)_{z}, (c)_{z} is covalently linked to (d)ₙ, and (d)ₙ is covalently linked to (b)_{y};
(ii) (a)ₓ, (c)_{z}, (d)ₙ and (b)_{y}, wherein (a)ₓ is covalently linked to (c)_{z}, (c)_{z} is covalently linked to (d)ₙ, and (d)ₙ is non-covalently linked to (b)_{y};
(iii) (a)ₓ, (d)ₙ, (c)_{z} and (b)_{y}, wherein (a)ₓ is covalently linked to (d)ₙ, (d)ₙ is covalently linked to (c)_{z}, and (c)_{z} is covalently linked to (b)_{y};
(iv) (a)ₓ, (d)ₙ, (c)_{z} and (b)_{y}, wherein (a)ₓ is non-covalently linked to (d)ₙ, (d)ₙ is non-covalently linked to (c)_{z}, and (c)_{z} is covalently linked to (b)_{y};
(v) (a)ₓ, (c)_{z}, (d)ₙ, and (b)_{y}, wherein (a)ₓ is covalently linked to (c)_{z} via a linker molecule, (c)_{z} is covalently linked to (d)ₙ via a linker molecule, and (d)ₙ is covalently linked to (b)_{y} via a linker molecule;
(vi) (a)ₓ, (c)_{z}, (d)ₙ, and (b)_{y}, wherein (a)ₓ is covalently linked to (c)_{z} via a linker molecule, (c)_{z} is covalently linked to (d)ₙ via a linker molecule, and (d)ₙ is non-covalently linked to (b)_{y};
(vii) (a)ₓ, (d)ₙ, (c)_{z} and (b)_{y}, wherein (a)ₓ is covalently linked to (d)ₙ via a linker molecule, (d)ₙ is covalently linked to (c)_{z} via a linker molecule and (c)_{z} is covalently linked to (b)_{y} via a linker molecule;
(viii) (a)ₓ, (d)ₙ, (c)_{z} and (b)_{y}, wherein (a)ₓ is non-covalently linked to (d)ₙ, (d)ₙ is non-covalently linked to (c)_{z}, and (c)_{z} is covalently linked to (b)_{y} via a linker molecule, or
(ix) (a)ₓ, (d)ₙ, (c)_{z} and (b)_{y}, wherein (a)ₓ is non-covalently linked to (d)ₙ via an adapter molecule that is covalently linked to (a)ₓ, (d)ₙ is non-covalently linked to (c)_{z} via an adapter molecule that is covalently linked to (c), and (c)_{z} is covalently linked to (b)_{y} via a linker molecule, and wherein
   x is an integer of 1 to 5, preferably of 1;
   y is an integer of 1 to 5; preferably of 1;
   z is an integer of 1 to 5; preferably of 1; and
   n is an integer of 1 to 50, preferably of 2, 3, 4, 5, 6, 7, 8, 9, or 10.

It is preferred that there are no other linkages, preferably no covalent linkages, between the respective modules other than the linkages specifically indicated above or below with respect to the more preferred embodiments.

Thus, conjugates according to the present invention are particularly preferred that carry module (b) in a terminal position, preferably in last (i.e., C-terminal) position, and wherein modules (a), (b) and (c), and compound (d) are completely covalently linked to each other or partially covalently linked to each other, e.g., conjugate: (a)ₓ, (c)_{z}, (d)ₙ and (b)_{y}, wherein (a)ₓ is covalently linked to (c)_{z}, (c)_{z} is covalently linked to (d)ₙ, and (d)ₙ is covalently linked to (b)_{y}; or conjugate: (a)ₓ, (c)_{z}, (d)ₙ and (b)_{y}, wherein (a)ₓ is covalently linked to (c)_{z}, (c)_{z} is covalently linked to (d)ₙ, and (d)ₙ is non-covalently linked to (b)_{y}. In these examples, module (b) is unhindered by the other modules (a) and (c) and by the compound (d). Module (b) is also not extended by linkages of other modules. Hence, steric or other undesired interactions can be avoided or at least minimized.

For in vivo applications, it is preferred to use conjugates that comprise module (b) in the C-terminal position, and wherein modules (a), (b) and (c), and compound (d) are completely covalently linked to each other and/or covalently linked to each other via a linker molecule, e.g. conjugate: (a)ₓ, (c)_{z}, (d)ₙ and (b)_{y}, wherein (a)ₓ is covalently linked to (c)_{z}, (c)_{z} is covalently linked to (d)ₙ, and (d)ₙ is covalently linked to (b)_{y}; or conjugate: (a)ₓ, (c)_{z}, (d)ₙ, and (b)_{y}, wherein (a)ₓ is covalently linked to (c)_{z} via a linker molecule, (c)_{z} is covalently linked to (d)ₙ via a linker molecule, and (d)ₙ is covalently linked to (b)_{y} via a linker molecule; or conjugate: (a)ₓ, (d)ₙ, (c)_{z} and (b)_{y}, wherein (a)ₓ is covalently linked to (d)ₙ, (d)ₙ is covalently linked to (c)_{z}, and (c)_{z} is covalently linked to (b)_{y}. These exemplary conjugates are more stable compared to conjugates that comprise modules and compounds that are only non-covalently linked or partially non-covalently linked to each other and, thus are more preferred for in vivo applications.

For in vitro applications, e.g. in cell culture, it is preferred to use conjugates that comprise module (b) in the C-terminal position, and wherein modules (a), (b) and (c) are only partially covalently linked, e.g. conjugate: (a)ₓ, (d)ₙ, (c)_{z} and (b)_{y}, wherein (a)ₓ is non-covalently linked to (d)ₙ, (d)ₙ is non-covalently linked to (c)_{z}, and (c)_{z} is covalently linked to (b)_{y} via a linker molecule. This exemplary conjugate is less complex and easier to synthesize and, thus, more preferred for in vitro applications as predominant test systems. Nucleic acid compounds in this exemplary conjugate can also more readily be exchanged in order to test libraries of compound molecules for their biological activity in cells. Thus, the conjugates of the invention are also useful in screening assays.

Conjugates are also preferred that comprise compound (d) in second or third position, and wherein compound (d) is directly covalently linked or indirectly covalently linked via a linkage molecule to modules (a) or (c), e.g. conjugate: (a)ₓ, (d)ₙ, (c)_{z} and (b)_{y}, wherein (a)ₓ is covalently linked to (d)ₙ, (d)ₙ is covalently linked to (c)_{z}, and (c)_{z} is covalently linked to (b)_{y}; or conjugate: (a)ₓ, (d)ₙ, (c)_{z} and (b)_{y}, wherein (a)ₓ is covalently linked to (d)ₙ via a linker molecule, (d)ₙ is covalently linked to (c)_{z} via a linker molecule and (c)_{z} is covalently linked to (b)_{y} via a linker molecule. These exemplary conjugates assure flexibility of compound (d). In addition, the linker molecules connecting compound (d) with modules (a) and (c) have a spacer function, which keeps modules (a) and (c) safely away from the compound (d). Thus, steric and other undesired interactions can be avoided or at least minimized.

More preferred are conjugates according to the present invention that comprise the following arrangement:

(a)ₓ, (d)ₙ, (c)_{z} and (b)_{y}, wherein (a)ₓ is covalently linked to (d)ₙ, (d)ₙ is covalently linked to (c)_{z}, and (c)_{z} is covalently linked to (b)_{y}, and wherein x is an integer of 1, n is an integer of 2 or 3, z is an integer of 1, and y is an integer of 1, or
(a)ₓ, (d)ₙ, (c)_{z} and (b)_{y}, wherein (a)ₓ is covalently linked to (d)ₙ via a linker molecule, (d)ₙ is covalently linked to (c)_{z} via a linker molecule and (c)_{z} is covalently linked to (b)_{y} via a linker molecule, and wherein x is an integer of 1, n is an integer of 2, 3, 4, 5, 6, 7, 8, 9, or 10, z is an integer of 1 and y is an integer of 1.

It is particularly preferred that the modules (a), (b), (c) and the compound (d) of the conjugate of the present invention are linked to each other in the following arrangements, wherein
(i) (a)ₓ is covalently linked to (c)_{z}, (c)_{z} is covalently linked to (d)ₙ, and (c)_{z} is covalently linked to (b)_{y};
(ii) (a)ₓ is covalently linked to (c)_{z}, (c)_{z} is non-covalently linked to (d)ₙ, and (c)_{z} is covalently linked to (b)_{y};
(iii) (a)ₓ is covalently linked to (d)ₙ, (a)ₓ is covalently linked to (c)_{z}, and (c)_{z} is covalently linked to (b)_{y};
(iv) (a)ₓ is non-covalently linked to (d)ₙ, (a)ₓ is covalently linked to (c), and (c)_{z} is covalently linked to (b)_{y};
(v) (a)ₓ is covalently linked to (c)_{z} via a linker molecule, (c)_{z} is covalently linked to (d)ₙ via a linker molecule, and (c)_{z} is covalently linked to (b)_{y} via a linker molecule;
(vi) (a)ₓ is covalently linked to (c)_{z} via a linker molecule, (c)_{z} is non-covalently linked to (d)ₙ via an adapter molecule that is covalently linked to (c)_{z}, and (c)_{z} is covalently linked to (b)_{y} via a linker molecule;
(vii) (a)ₓ is covalently linked to (d)ₙ via a linker molecule, (a)ₓ is covalently linked to (c)_{z} via a linker molecule and (c)_{z} is covalently linked to (b)_{y} via a linker molecule; or
(viii) (a)ₓ is non-covalently linked to (d)ₙ via an adapter molecule that is covalently linked to (a)ₓ, (a)ₓ is covalently linked to (c)_{z} via a linker molecule, and (c)_{z} is covalently linked to (b)_{y} via a linker molecule.

It is preferred that there are no other linkages, preferably no covalent linkages, between the respective modules other than the covalent linkages and non-covalent linkages, respectively, specifically indicated above.

More preferred, the modules (a), (b), (c) and the compound (d) of the conjugate of the present invention are linked to each other in the following arrangements (in each case a structural drawing indicating the respective modules and their spatial arrangements is also provided), wherein
(i) (a)ₓ is covalently linked to (c)_{z}, (c)_{z}, is covalently linked to (d)ₙ, and (c)_{z} is covalently linked to (b)_{y}; (a)ₓ-((c)_{z}-(b)_{y})-(d)ₙ,
(ii) (a)ₓ is covalently linked to (c)_{z}, (c)_{z} is non-covalently linked to (d)ₙ, and (c)_{z} is covalently linked to (b)_{y}; (a)ₓ-((c)_{z}-(b)_{y})—(d)ₙ,
(iii) (a)ₓ is covalently linked to (d)ₙ, (a)ₓ is covalently linked to (c)_{z}, and (c)_{z} is covalently linked to (b)_{y}; ((a)ₓ-(d)ₙ)-(c)_{z}-(b)_{y},
(iv) (a)ₓ is non-covalently linked to (d)ₙ, (a)ₓ is covalently linked to (c)_{z}, and (c)_{z} is covalently linked to (b)_{y}; ((a)ₓ-(d)ₙ)-(c)_{z}-(b)_{y},
(v) (a)ₓ is covalently linked to (c)_{z} via a linker molecule, (c)_{z} is covalently linked to (d)ₙ via a linker molecule, and (c)_{z} is covalently linked to (b)_{y} via a linker molecule; (a)ₓ-L-((c)_{z}-L-(b)_{y})-L-(d)ₙ,
(vi) (a)ₓ is covalently linked to (c)_{z} via a linker molecule, (c)_{z} is non-covalently linked to (d)ₙ via an adapter molecule that is covalently linked to (c)_{z}, and (c)_{z} is covalently linked to (b)_{y} via a linker molecule; (a)ₓ-L-((c)_{z}-L-(b)_{y})-A---(d)ₙ,
(vii) (a)ₓ is covalently linked to (c)_{z} via a linker molecule, (c)_{z} is non-covalently linked to (d)ₙ via an adapter molecule that is covalently linked via a linker to (c)_{z}, and (c)_{z} is covalently linked to (b)_{y} via a linker molecule; (a)ₓ-L-((c)_{z}-L-(b)_{y})-L-A---(d)ₙ,
(viii) (a)ₓ is covalently linked to (c)_{z}, (c)_{z} is covalently linked to (d)ₙ via a linker molecule, and (c)_{z} is covalently linked to (b)_{y} via a linker molecule; (a)ₓ-((c)_{z}-L-(b)_{y})-L-(d)ₙ,
(ix) (a)ₓ is covalently linked to (c)_{z}, (c)_{z} is non-covalently linked to (d)ₙ via an adapter molecule that is covalently linked to (c)_{z}, and (c)_{z} is covalently linked to (b)_{y} via a linker molecule; (a)ₓ-((c)_{z}-L-(b)_{y})-A---(d)ₙ,
(x) (a)ₓ is covalently linked to (c)_{z}, (c)_{z} is non-covalently linked to (d)ₙ via an adapter molecule that is covalently linked via a linker to (c)_{z}, and (c)_{z} is covalently linked to (b)_{y} via a linker molecule; (a)ₓ-((c)_{z}-L-(b)_{y})-L-A---(d)ₙ,
(xi) (a)ₓ is covalently linked to (c)_{z}, (c)_{z} is covalently linked to (d)ₙ via a linker molecule, and (c)_{z} is covalently linked to (b)_{y}; (a)ₓ-((c)_{z}-(b)_{y})-L-(d)ₙ,
(xii) (a)ₓ is covalently linked to (c)_{z}, (c)_{z} is non-covalently linked to (d)ₙ via an adapter molecule that is covalently linked to (c)_{z}, and (c)_{z} is covalently linked to (b)_{y}; (a)ₓ-((c)_{z}-(b)_{y})-A---(d)ₙ,
(xiii) (a)ₓ is covalently linked to (c)_{z}, (c)_{z} is non-covalently linked to (d)ₙ via an adapter molecule that is covalently linked via a linker to (c)_{z}, and (c)_{z} is covalently linked to (b)_{y}; (a)ₓ-((c)_{z}-(b)_{y})-L-A---(d)ₙ,
(xiv) (a)ₓ is covalently linked to (c)_{z} via a linker, (c)_{z} is covalently linked to (d)ₙ via a linker molecule, and (c)_{z} is covalently linked to (b)_{y}; (a)ₓ-L-((c)_{z}-(b)_{y})-L-(d)ₙ,
(xv) (a)ₓ is covalently linked to (c)_{z} via a linker, (c)_{z} is non-covalently linked to (d)ₙ via an adapter molecule that is covalently linked to (c)_{z}, and (c)_{z} is covalently linked to (b)_{y}; (a)ₓ-L-((c)_{z}-(b)_{y})-A---(d)ₙ,
(xvi) (a)ₓ is covalently linked to (c)_{z} via a linker, (c)_{z} is non-covalently linked to (d)ₙ via an adapter molecule that is covalently linked via a linker to (c)_{z}, and (c)_{z} is covalently linked to (b)_{y}; (a)ₓ-L-((c)_{z}-(b)_{y})-L-A---(d)ₙ,
(xvii) (a)ₓ is covalently linked to (c)_{z} via a linker, (c)_{z} is covalently linked to (d)ₙ, and (c)_{z} is covalently linked to (b)_{y}; (a)ₓ-L-((c)_{z}-(b)_{y})-(d)ₙ,
(xviii) (a)ₓ is covalently linked to (c)_{z} via a linker, (c)_{z} is non-covalently linked to (d)ₙ via an adapter molecule that is covalently linked to (c)_{z}, and (c)_{z} is covalently linked to (b)_{y}; (a)ₓ-L-((c)_{z}-(b)_{y})-A---(d)ₙ,
(xix) (a)ₓ is covalently linked to (c)_{z} via a linker, (c)_{z} is covalently linked to (d)ₙ, and (c)_{z} is covalently linked to (b)_{y}; (a)ₓ-((c)_{z}-L-(b)_{y})-(d)ₙ,
(xx) (a)ₓ is covalently linked to (c)_{z} via a linker, (4 is non-covalently linked to (d)ₙ via an adapter molecule that is covalently linked to (c)_{z}, and (c)_{z} is covalently linked to (b)_{y}; (a)ₓ-((c)_{z}-L-(b)_{y})-A---(d)ₙ,
(xxi) (a)ₓ is covalently linked to (d)ₙ via a linker molecule, (a)ₓ is covalently linked to (c)_{z} via a linker molecule and (c)_{z} is covalently linked to (b)_{y} via a linker molecule; ((a)ₓ-L-(d)ₙ)-L-(c)_{z}-L-(b)_{y},
(xxii) (a)ₓ is non-covalently linked to (d)ₙ via an adapter molecule that is covalently linked to (a)ₓ, (a)ₓ is covalently linked to (c)_{z} via a linker molecule, and (c)_{z} is covalently linked to (b)_{y} via a linker molecule; ((a)ₓ-A---(d)ₙ)-L-(c)_{z}-L-(b)_{y},
(xxiii) (a)ₓ is non-covalently linked to (d)ₙ via an adapter molecule that is covalently linked to (a)ₓ, (a)ₓ is covalently linked to (c)_{z} via a linker molecule, and (c)_{z} is covalently linked to (b)_{y}; ((a)ₓ-A---(d)ₙ)-L-(c)_{z}-(b)_{y},
(xxiv) (a)ₓ is non-covalently linked to (d)ₙ via an adapter molecule that is covalently linked via a linker to (a)ₓ, (a)ₓ is covalently linked to (c)_{z} via a linker molecule, and (c)_{z} is covalently linked to (b)_{y} via a linker molecule; ((a)ₓ-L-A---(d)ₙ)-L-(c)_{z}-L-(b)_{y},
(xxv) (a)ₓ is non-covalently linked to (d)ₙ via an adapter molecule that is covalently linked linked via a linker to (a)ₓ, (a)ₓ is covalently linked to (c)_{z} via a linker molecule, and (c)_{z} is covalently linked to (b)_{y}; ((a)ₓ-L-A---(d)ₙ)-L-(c)_{z}-(b)_{y},
(xxvi) (a)ₓ is covalently linked to (d)ₙ via a linker molecule, (a)ₓ is covalently linked to (c)_{z} and (c)_{z} is covalently linked to (b)_{y} via a linker molecule; ((a)ₓ-L-(d)ₙ)-(c)_{z}-L-(b)_{y},
(xxvii) (a)ₓ is non-covalently linked to (d)ₙ via an adapter molecule that is covalently linked to (a)ₓ, (a)ₓ is covalently linked to (c)_{z}, and (c)_{z} is covalently linked to (b)_{y} via a linker molecule; ((a)_{X}-A---(d)ₙ)-(c)_{z}-L-(b)_{y},
(xxviii) (a)ₓ is non-covalently linked to (d)ₙ via an adapter molecule that is covalently linked to (a)ₓ, (a)ₓ is covalently linked to (c)_{z}, and (c)_{z} is covalently linked to (b)_{y}; ((a)ₓ-A—(d)ₙ)-(c)_{z}-(b)_{y},
(xxix) (a)ₓ is non-covalently linked to (d)ₙ via an adapter molecule that is covalently linked via a linker to (a)ₓ, (a)ₓ is covalently linked to (c)_{z}, and (c)_{z} is covalently linked to (b)_{y} via a linker molecule; ((a)_{X}-L-A---(d)ₙ-(c)_{z}-L-(b)_{y},
(xxx) (a)ₓ is non-covalently linked to (d)ₙ via an adapter molecule that is covalently linked linked via a linker to (a)ₓ, (a)ₓ is covalently linked to (c)_{z}, and (c)_{z} is covalently linked to (b)_{y}, ((a)ₓ-L-A---(d)ₙ)-(c)_{z}-(b)_{y},
(xxxi) (a)ₓ is non-covalently linked to (d)ₙ via an adapter molecule that is covalently linked to (a)ₓ, (a)ₓ is covalently linked to (c-b)ₖ, via a linker molecule; ((a)ₓ-A---(d)ₙ)-L-(c-b)ₖ,
(xxxii) (a)ₓ is non-covalently linked to (d)ₙ via an adapter molecule that is covalently linked linked via a linker to (a)ₓ, (a)ₓ is covalently linked to (c-b)ₖ via a linker molecule; ((a)ₓ-L-A---(d)ₙ)-L-(c-b)ₖ,
(xxxiii) (a)ₓ is non-covalently linked to (d)ₙ via an adapter molecule that is covalently linked to (a)ₓ, (a)ₓ is covalently linked to (c-b)ₖ; ((a)ₓ-A---(d)ₙ)-(c-b)ₖ, or
(xxxiv) (a)ₓ is non-covalently linked to (d)ₙ via an adapter molecule that is covalently linked linked via a linker to (a)ₓ, (a)ₓ is covalently linked to (c-b)ₖ; ((a)ₓ-L-A---(d)ₙ)-(c-b)ₖ,
   x is an integer of 1, 2, 3, 4, or 5, preferably of 1;
   y is an integer of 1, 2, 3, 4, or 5, preferably of 1;
   z is an integer of 1, 2, 3, 4, or 5, preferably of 1;
   k is an integer of 1, 2, 3, 4, or 5, preferably of 1; and
   n is an integer of 1 to 50, i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, preferably of 1, 2, 3, 4, 5, 6, 7, 8, 9, or 10;
   A is an adapter within above-defined meaning;
   (c-b) is used to indicate an embodiment according to the second aspect of the invention discussed in more detail below, wherein modules (c-b) are comprised within one peptide, protein or multisubunit complex;
   - indicates a covalent bond; and
   --- indicates a non-covalent bond.

It is preferred that there are no other linkages, preferably no covalent linkages, between the respective modules other than the covalent linkages and non-covalent linkages, respectively, specifically indicated above. Additionally it is preferred that the C-terminus of module (b) is accessible.

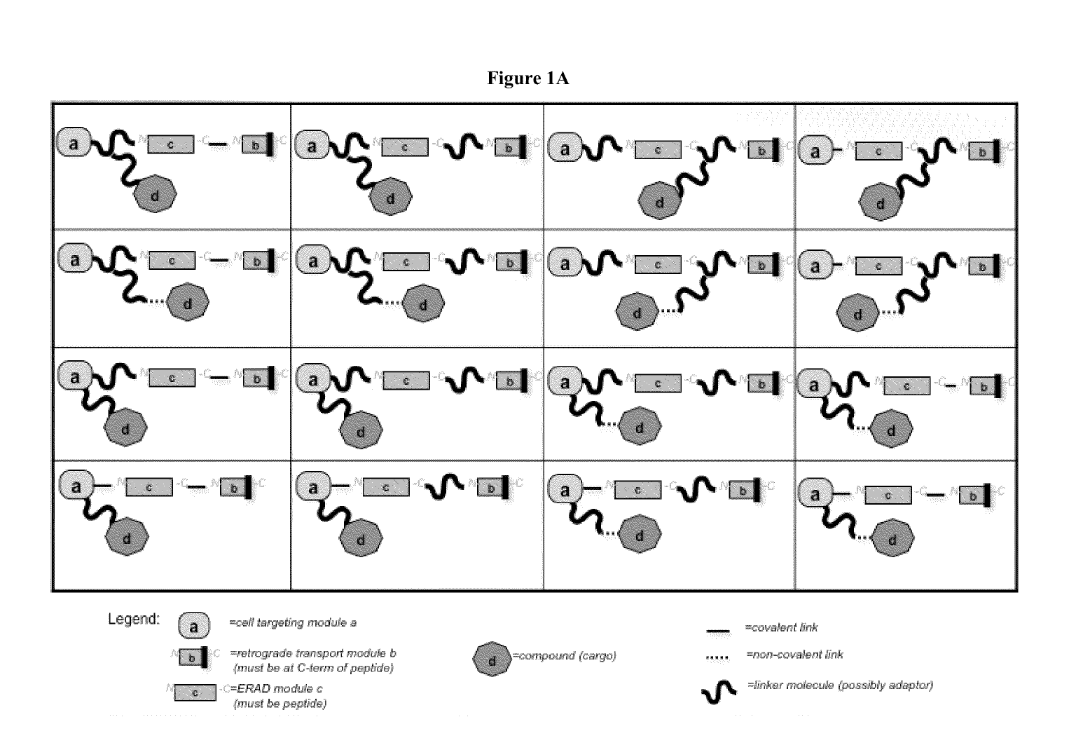

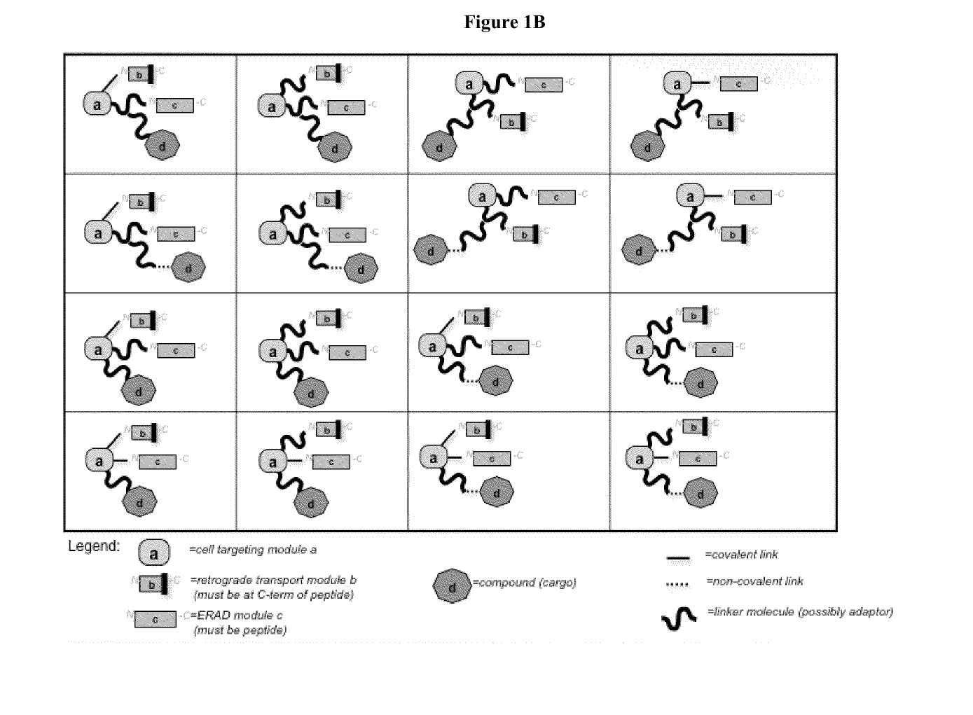

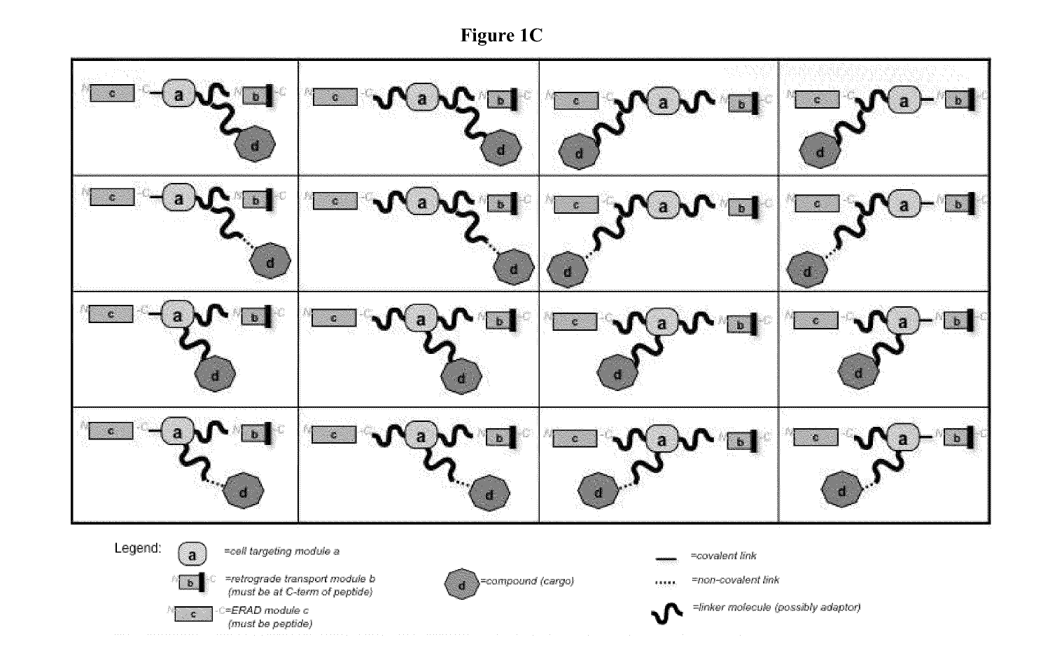

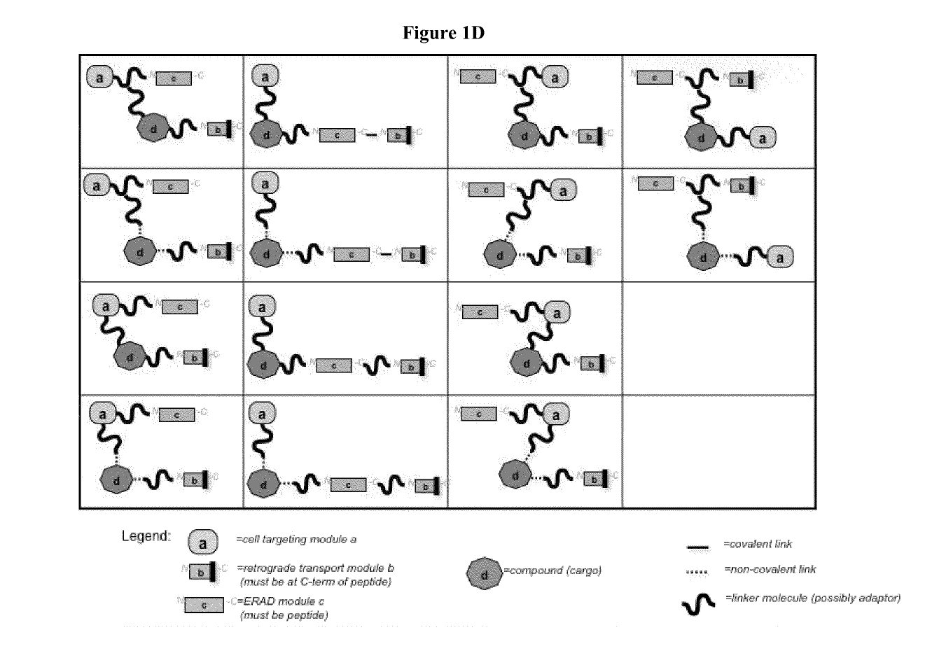

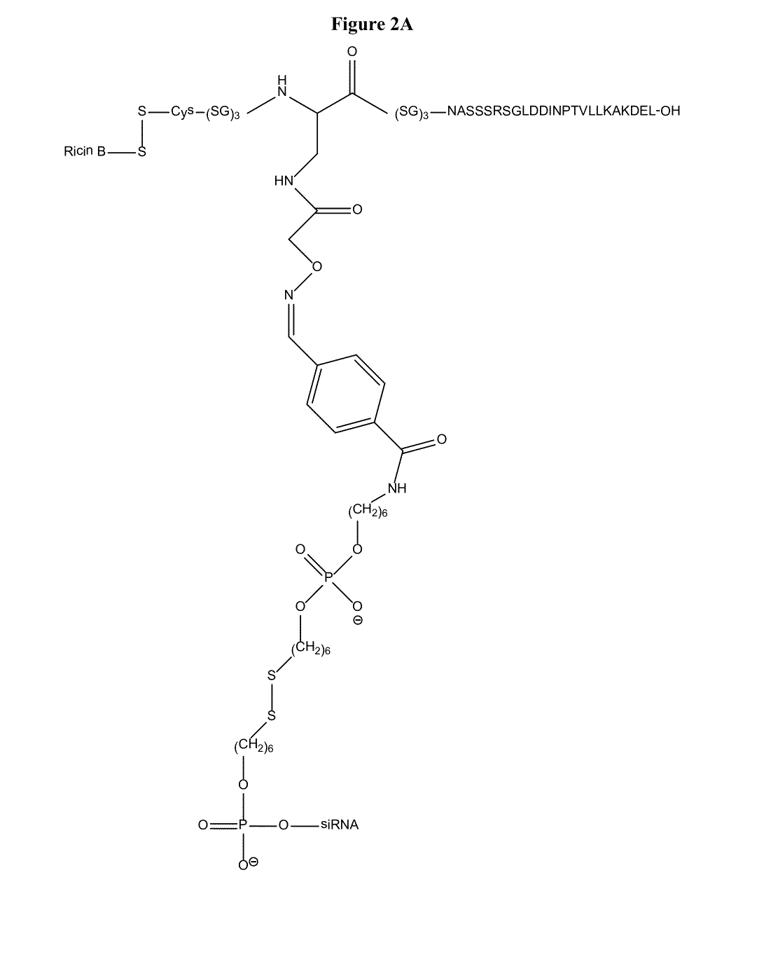

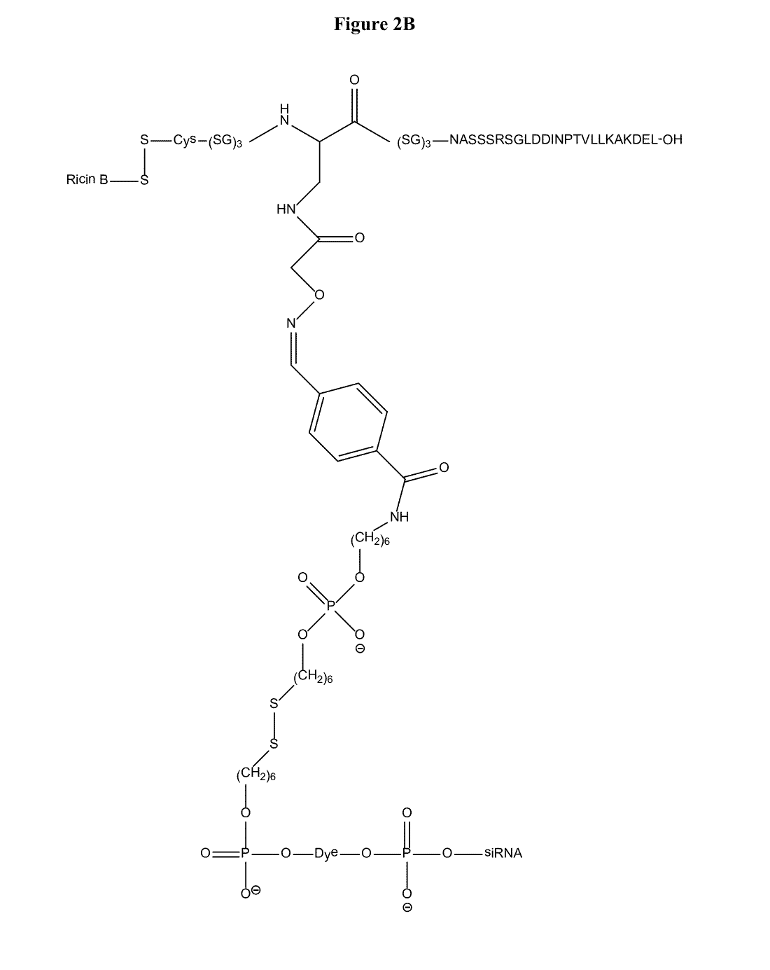

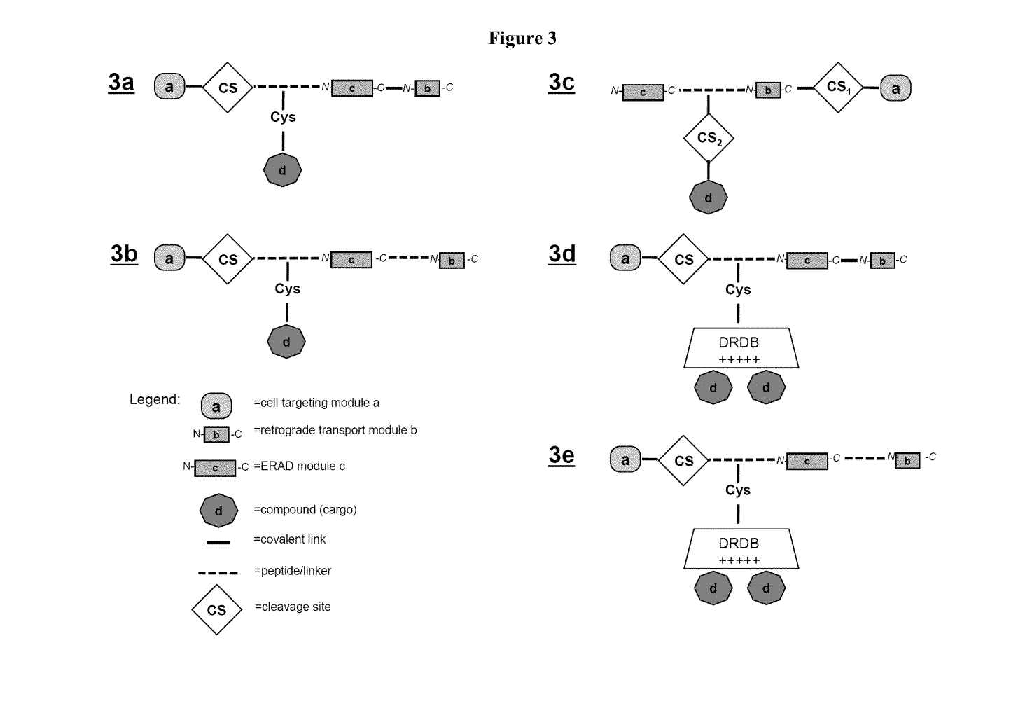

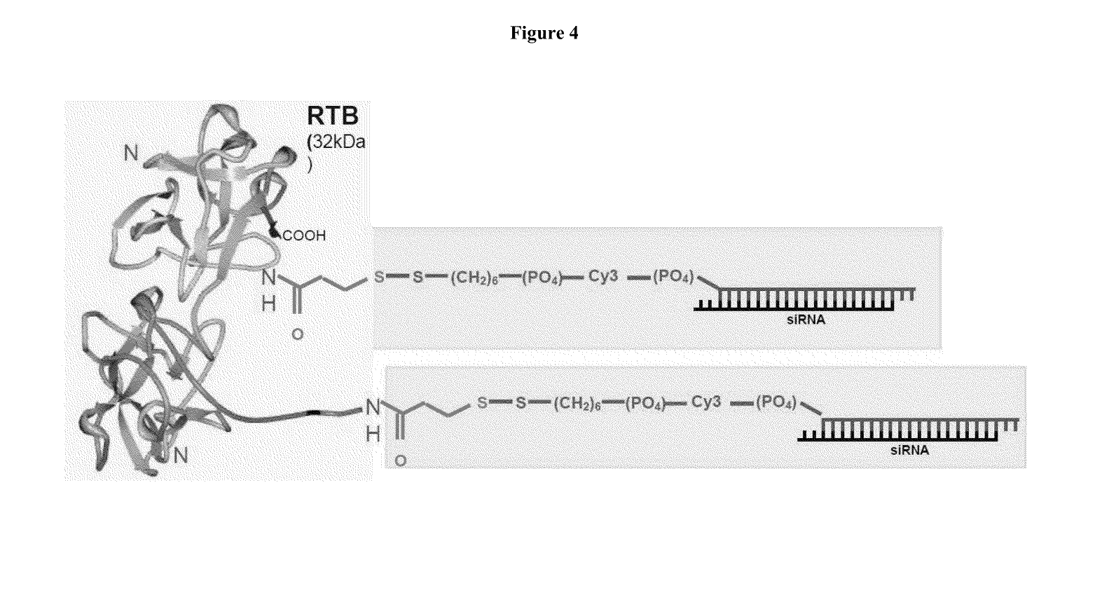

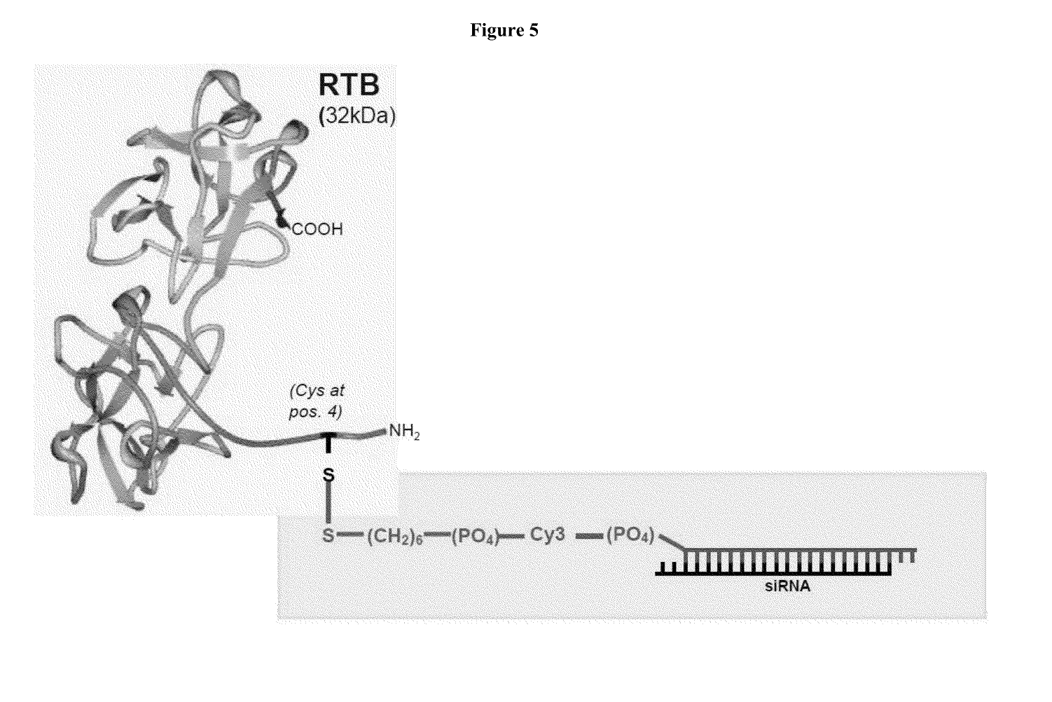

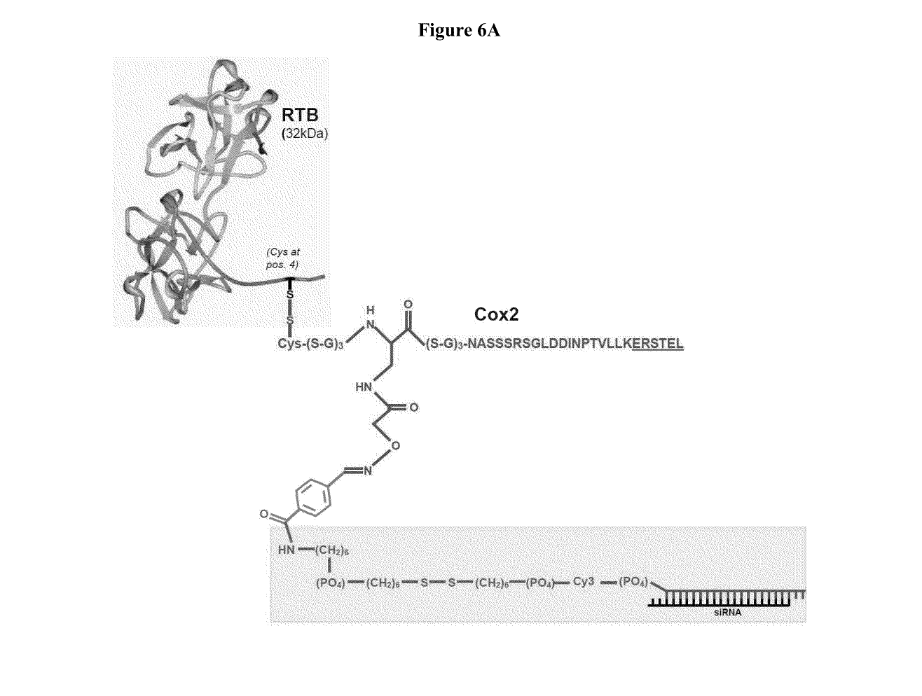

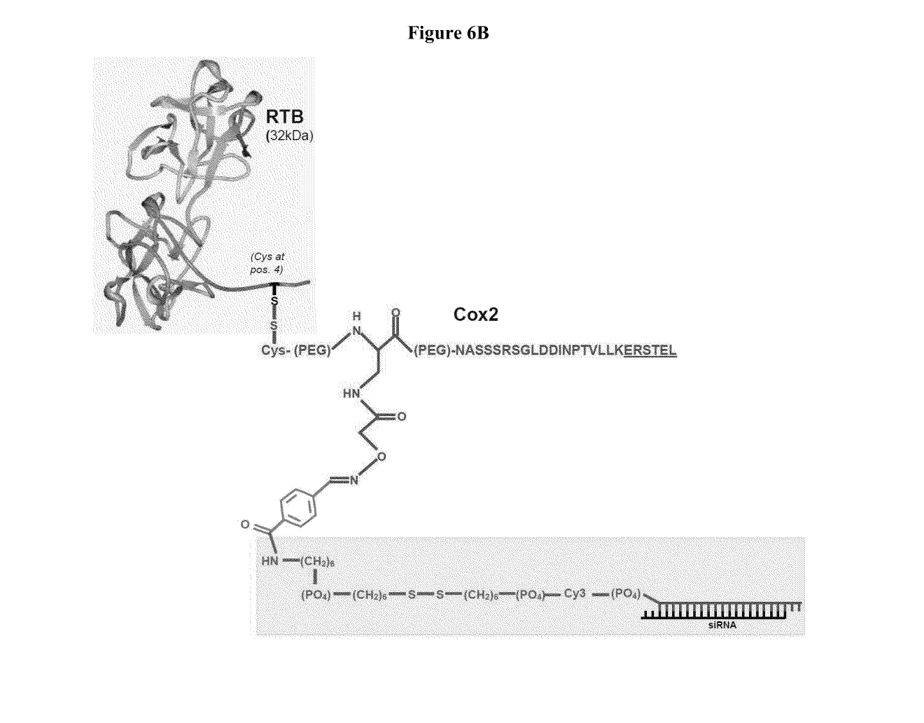

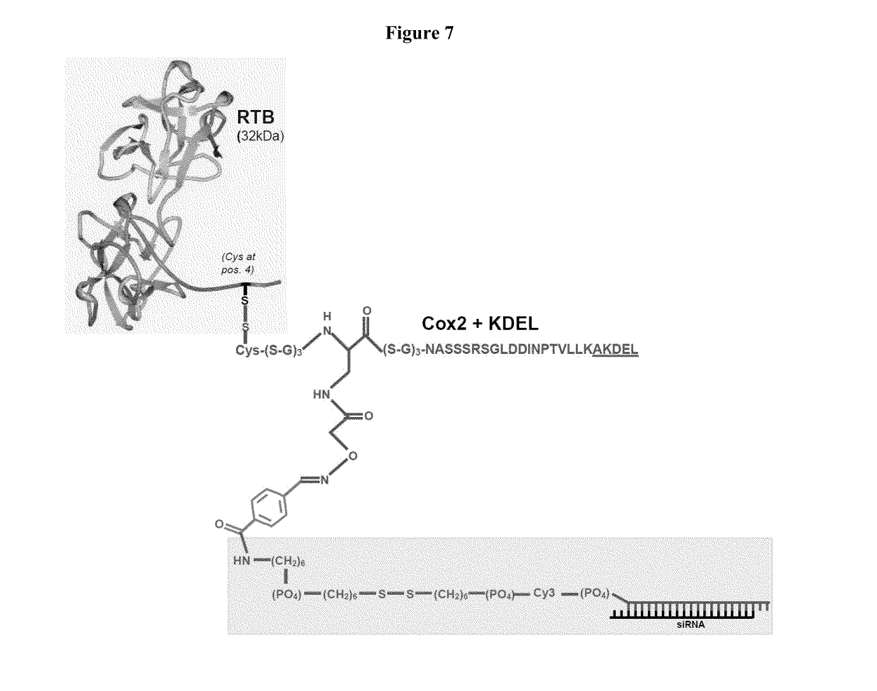

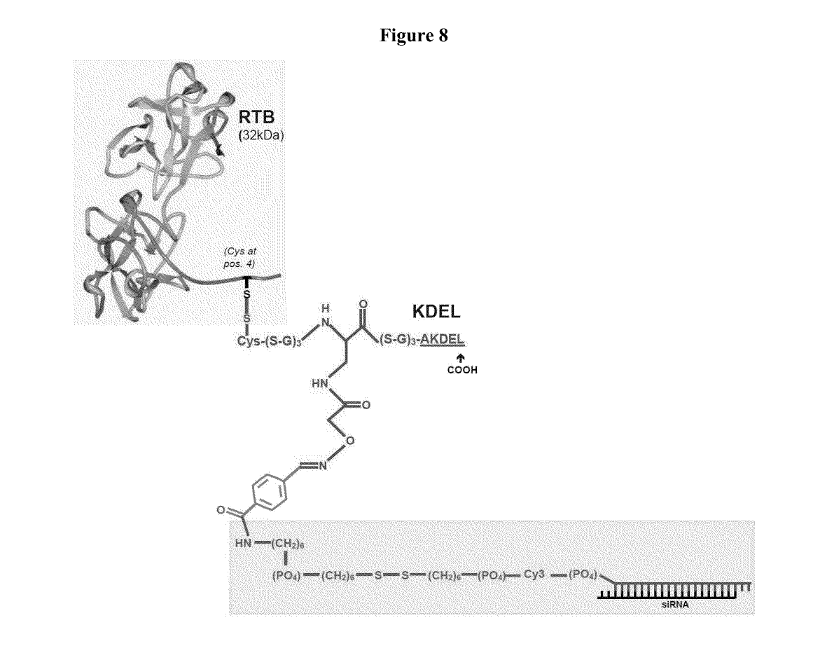

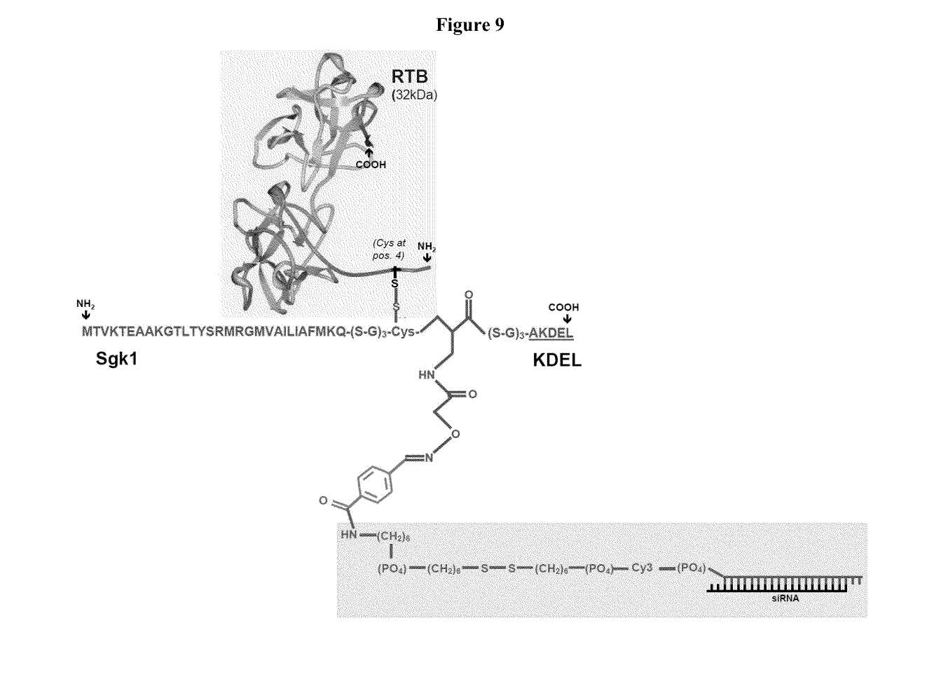

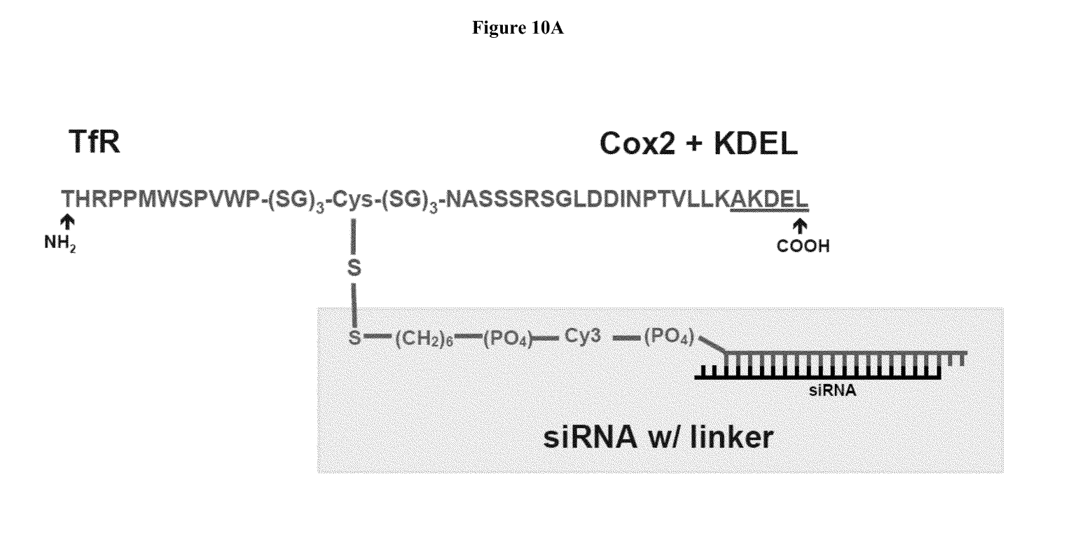

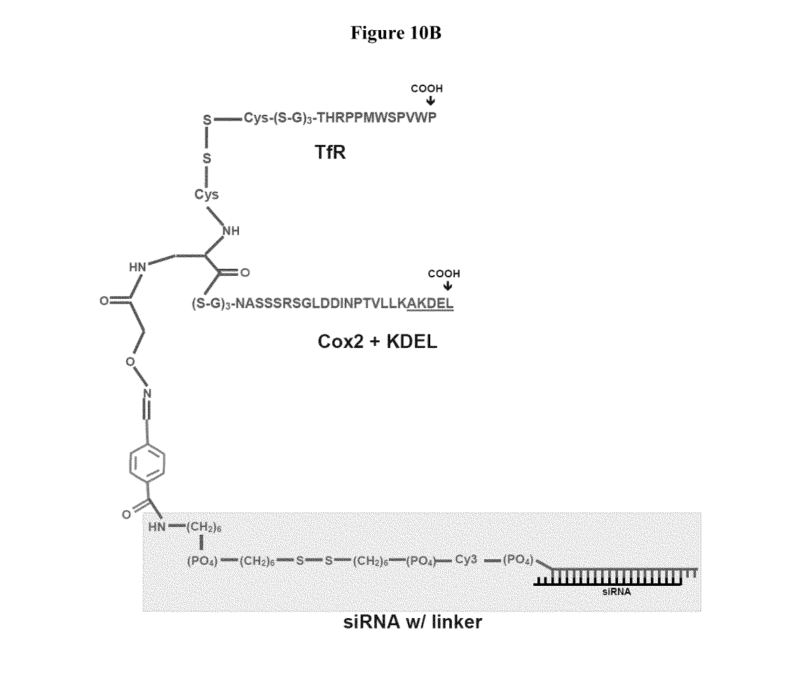

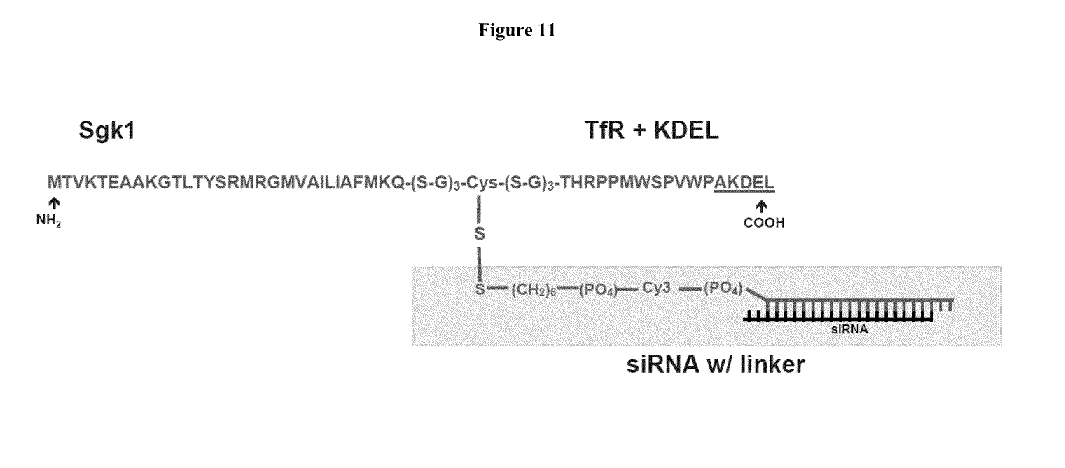

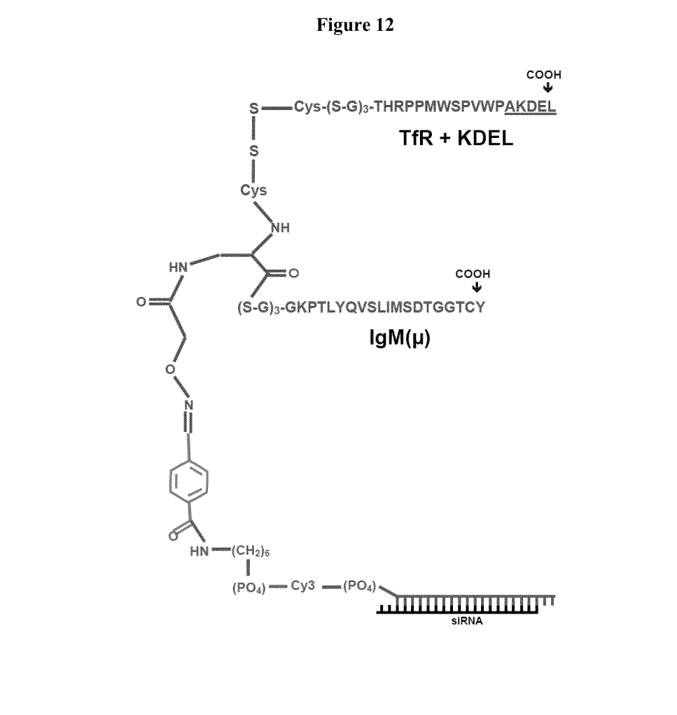

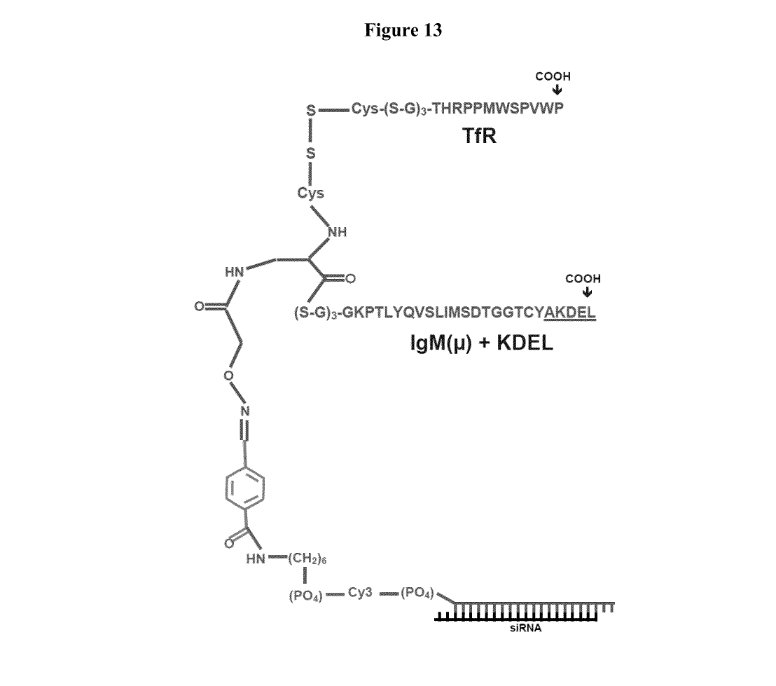

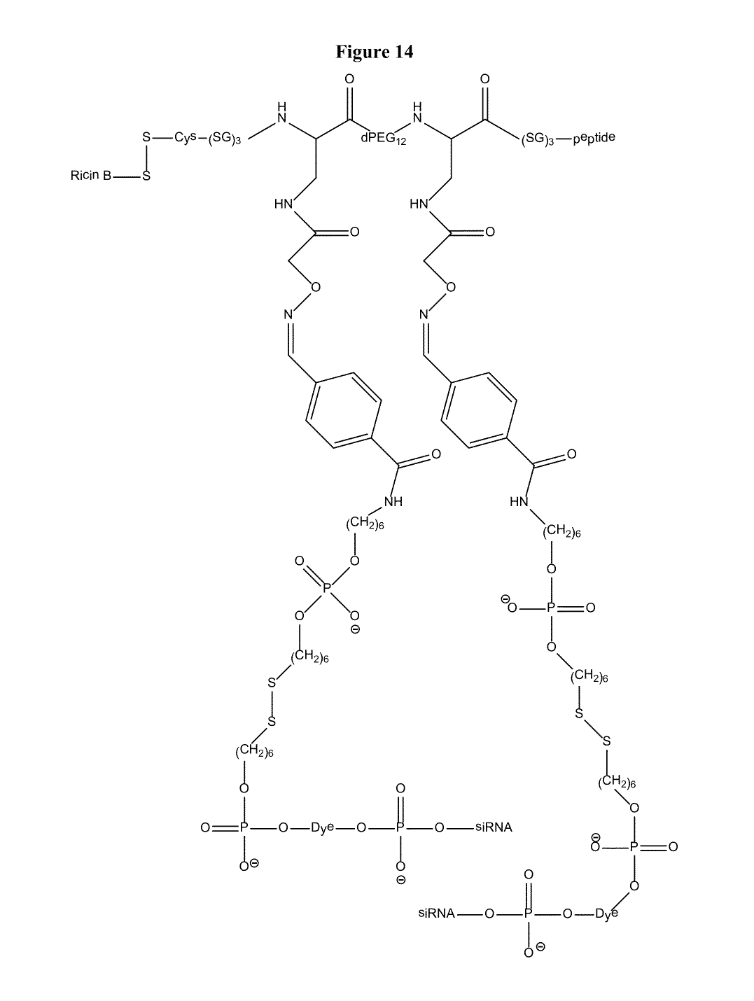

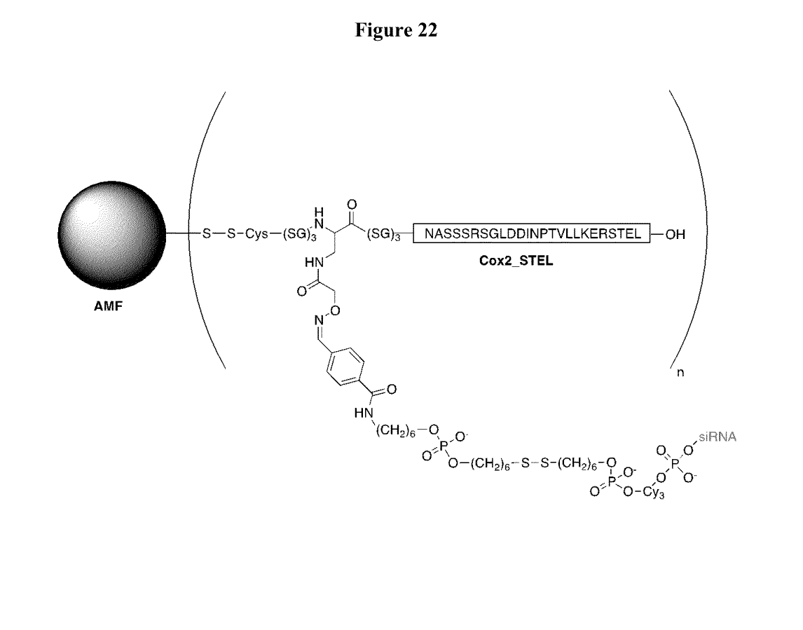

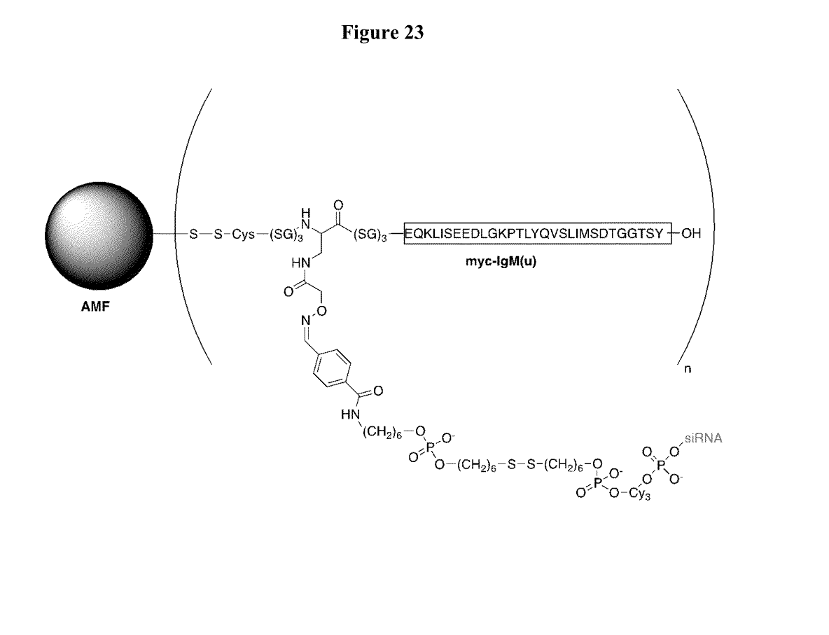

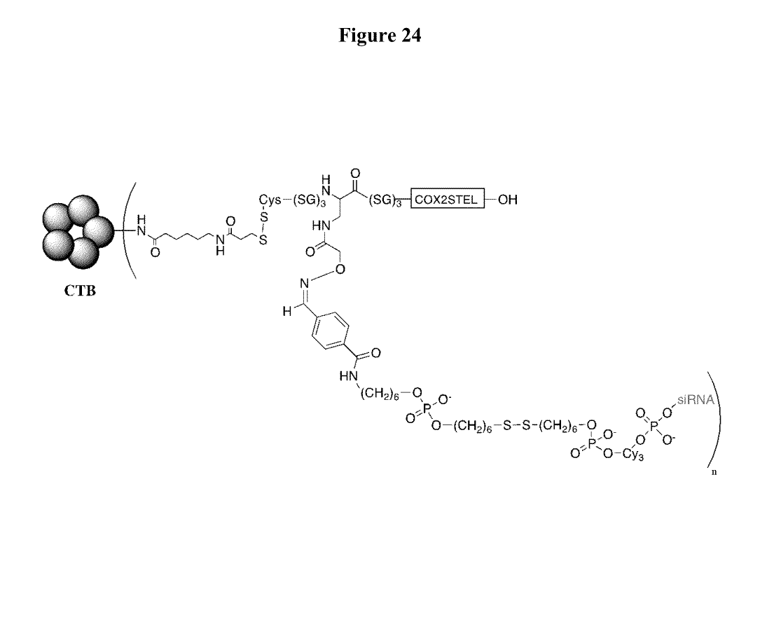

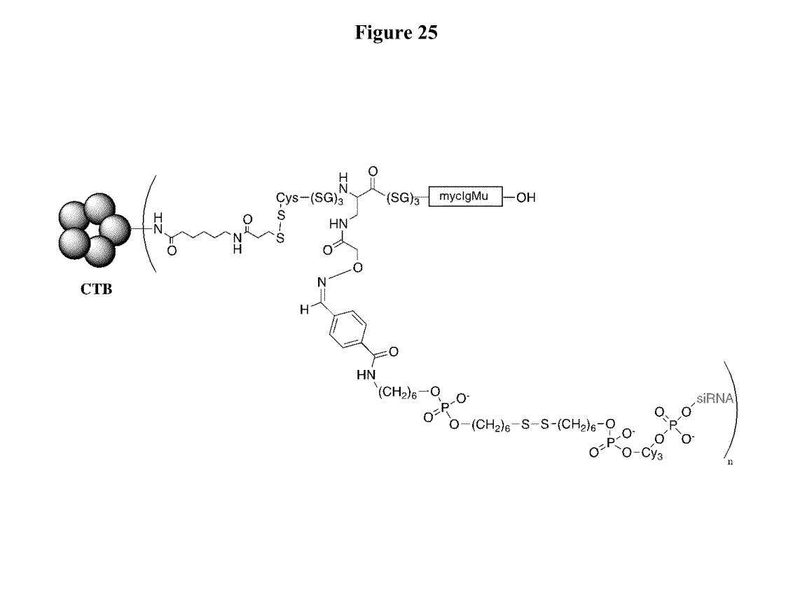

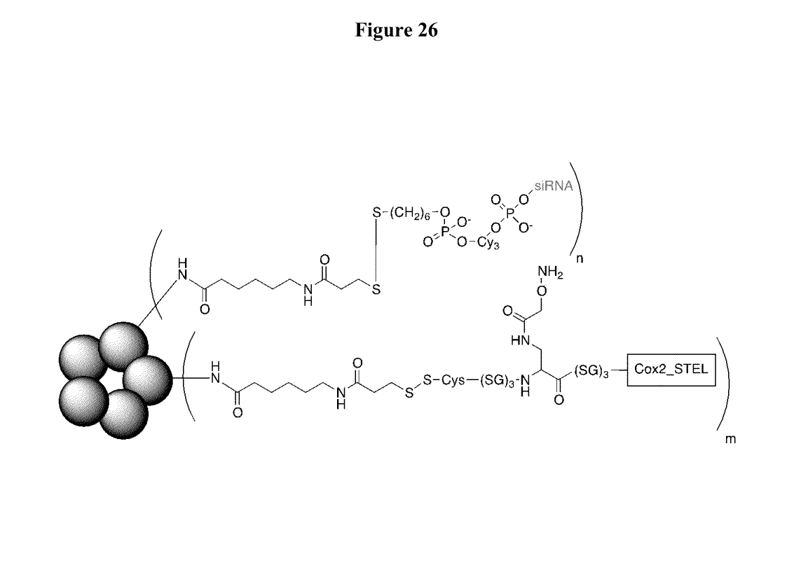

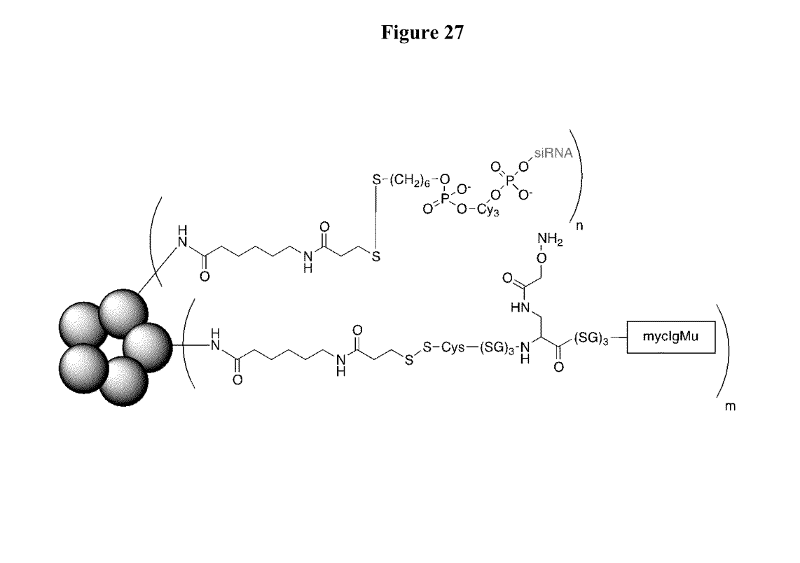

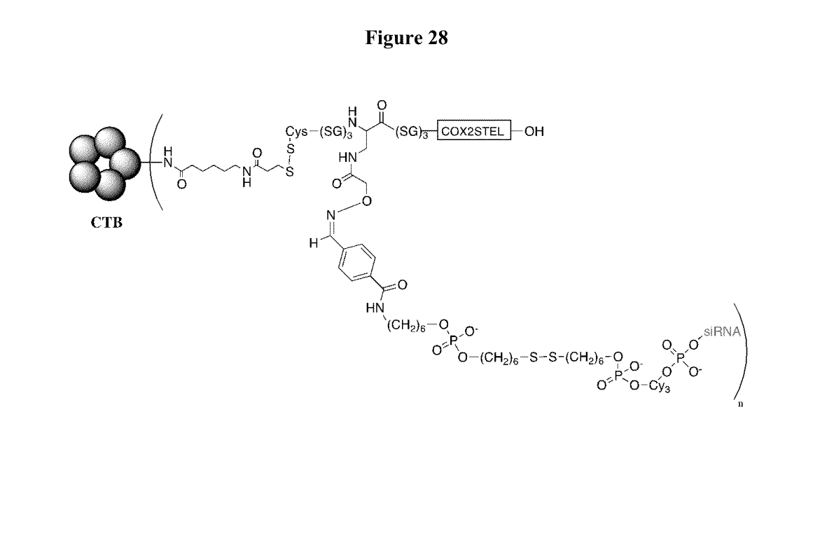

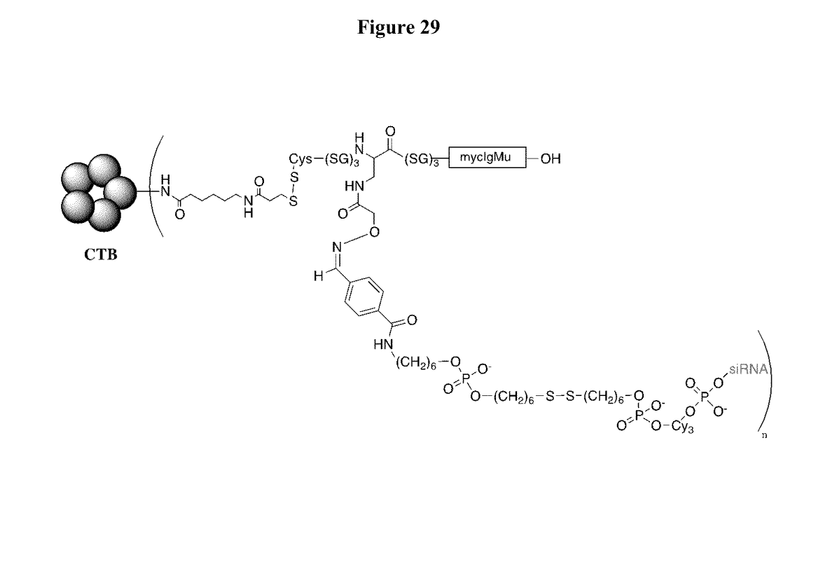

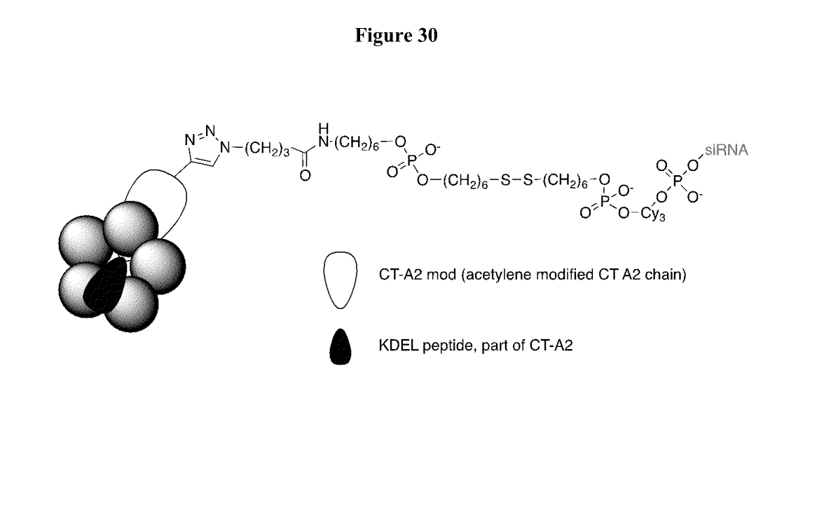

Preferred embodiments of the conjugate of the present invention are illustrated in FIGS. 1 (A) to (D), FIGS. 2 (A) and (B), FIGS. 3 (A) to (E), FIG. 4, FIG. 5, FIGS. 6 (A) and (B), FIG. 7, FIG. 8, FIG. 9, FIGS. 10 (A) and (B), FIG. 11, FIG. 12, FIG. 13, FIG. 14, 22, FIG. 23, FIG. 24, FIG. 25, FIG. 26, FIG. 27, FIG. 28, FIG. 29 and FIG. 30. FIGS. 1 (A) to (D) illustrate preferred embodiments of the conjugate of the present invention, wherein the modules, either separately among each other, or together with the compound (d), may be linked either covalently, non-covalently, via an adapter molecule or via a linker molecule that optimally comprises an adapter molecule. FIGS. 2 (A) and (B), FIGS. 3 (A) to (E), FIG. 4, FIG. 5, FIGS. 6 (A) and (B), FIG. 7, FIG. 8, FIG. 9, FIGS. 10 (A) and (B), FIG. 11, FIG. 12, FIG. 13, FIG. 14, FIG. 22, FIG. 23, FIG. 24, FIG. 25, FIG. 26, FIG. 27, FIG. 28, and FIG. 29 illustrate additional preferred embodiments of a conjugate of the present invention as described herein and in the Examples below.

In another preferred embodiment, the linker molecule, e.g. a peptide, a modified peptide, an amino acid residue or a modified amino acid residue, of the conjugate of the present invention that covalently links the at least one module (a) and/or the at least one module (b) and/or the at least one module (c) and/or the at least one compound (d), arranged in any combination, order, or stoichiometry to each other, further comprises
(i) at least one branch point, preferably a cysteine side chain, a lysine side chain, or an unnatural amino acid containing an aminooxy moiety on the side chain, and/or
(ii) at least one cleavage site, preferably an endosomal enzyme, a trans-Golgi network enzyme, a Golgi enzyme, an ER enzyme, a cytosolic enzyme or a nuclear enzyme cleavage site.

The term “branch point” in the context of the present invention means a position in a linker molecule, e.g. in a peptide linker, preferably an amino acid side chain, to which molecules, preferably a compound, an adapter molecule, a linker covalently attached to a compound, a linker covalently attached to an adapter, can be linked or coupled.

Preferred examples of arrangements of modules (a), (b), (c) and (d) comprising linkers with branch points (LB) and linkers (L) are as follows:
(i) (a)ₓ-(LB-(d)ₙ)-(c)_{z}-(b)_{y};
(ii) (a)ₓ-(LB-L-(d)ₙ)-(c)_{z}-(b)_{y};
(iii) (a)ₓ-(LB-(d)ₙ)-(c)_{z}-L-(b)_{y};
(iv) (a)ₓ-(LB-L-(d)ₙ)-(c)_{z}-L-(b)_{y};
(v) (a)ₓ-(LB-A---(d)ₙ)-(c)_{z}-(b)_{y};
(vi) (a)ₓ-(LB-L-A---(d)ₙ)-(c)_{z}-(b)_{y};
(vii) (a)ₓ-(LB-A---(d)ₙ)-(c)_{z}-L-(b)_{y};
(viii) (a)ₓ-(LB-L-A---(d)ₙ)-(c)_{z}-L-(b)_{y};
(ix) (a)ₓ-(c)_{z}-(LB-(d)ₙ)-(b)_{y};
(x) (a)ₓ-(c)_{z}-(LB-L-(d)ₙ)-(b)_{y};
(xi) (a)ₓ-L-(c)_{z}-(LB-(d)ₙ)-(b)_{y};
(xii) (a)ₓ-L-(c)_{z}-(LB-L-(d)ₙ)-(b)_{y};
(xiii) (a)ₓ-(c)_{z}-(LB-A---(d)ₙ)-(b)_{y};
(xiv) (a)ₓ-(c)_{z}-(LB-A---(d)ₙ)-(b)_{y};
(xv) (a)ₓ-L-(c)_{z}-(LB-A---(d)ₙ)-(b)_{y};
(xvi) (a)ₓ-L-(c)_{z}-(LB-A---(d)ₙ)-(b)_{y};
(xvii) (a)ₓ-(LB-(d)ₙ)-(c)_{z}-(LB-(d)ₙ)-(b)_{y};
(xviii) (a)ₓ-(LB-L-(d)ₙ)-(c)_{z}-(LB-(d)ₙ)-(b)_{y};
(xix) (a)ₓ-(LB-(d)ₙ)-(c)_{z}-(LB-L-(d)ₙ)-(b)_{y};
(xx) (a)ₓ-(LB-L-(d)ₙ)-(c)_{z}-(LB-L-(d)ₙ)-(b)_{y};
(xxi) (a)ₓ-(LB-A---(d)ₙ)-(c)_{z}-(LB-(d)ₙ)-(b)_{y};
(xxii) (a)ₓ-(LB-L-A---(d)ₙ)-(c)_{z}-(LB-(d)ₙ)-(b)_{y};
(xxiii) (a)ₓ-(LB-A---(d)ₙ)-(c)_{z}-(LB-L-(d)ₙ)-(b)_{y};
(xxiv) (a)ₓ-(LB-L-A---(d)ₙ)-(c)_{z}-(LB-L-(d)ₙ)-(b)_{y};
(xxv) (a)ₓ-(LB-(d)ₙ)-(c)_{z}-(LB-A---(d)ₙ)-(b)_{y};
(xxvi) (a)ₓ-(LB-L-(d)ₙ)-(c)_{z}-(LB-A---(d)ₙ) b)_{y};
(xxvii) (a)ₓ-(LB-(d)ₙ)-(c)_{z}-(LB-L-A---(d)ₙ)-b)_{y};
(xxviii) (a)ₓ-(LB-L-(d)ₙ)-(c)_{z}-(LB-L-A---(d)ₙ)b)_{y};
(ixxx) (a)ₓ-(LB-(d)ₙ)-(c-b)ₖ;
(xxx) (a)ₓ-(LB-L-(d)ₙ)-(c-b)ₖ;
(xxxi) (a)ₓ-(LB-A---(d)ₙ)-(c-b)ₖ;
(xxxii) (a)ₓ-(LB-L-A---(d)ₙ-(c-b)ₖ;
(xxxiii) (a)ₓ-(LB-(c)_{z})-(d)ₙ-(b)_{y};
(xxxiv) (a)ₓ-(LB-L-(c)_{z})-(d)ₙ-(b)_{y};
(xxxv) (a)ₓ-(LB-(c)_{z})-(d)ₙ-L-(b)_{y};
(xxxvi) (a)ₓ-(LB-L-(c)_{z})-(d)ₙ-L-(b)_{y};
(xxxvii) (a)ₓ-(d)ₙ-(LB-(c)_{z})-(b)_{y};
(xxxviii) (a)ₓ-(d)ₙ-(LB-L-(c)_{z})-(b)_{y};
(xxxxiv) (a)ₓ-L-(d)ₙ-(LB-(c)_{z})-(b)_{y};
(xxxv) (a)ₓ-L-(d)ₙ-(LB-L-(c)_{z})-(b)_{y};
(xxxvi) (a)ₓ-(LB-(c)_{z})-(d)ₙ-(LB-(c)_{z})-(b)_{y};
(xxxvii) (a)ₓ-(LB-L-(c)_{z})-(d)ₙ-(LB-(c)_{z})-(b)_{y};
(xxxviii) (a)ₓ-(LB-(c)_{z})-(d)ₙ-(LB-L-(c)_{z})-(b)_{y};
(xxxix) (a)ₓ-(LB-L-(c)_{z})-(d)ₙ-(LB-L-(c)_{z})-(b)_{y};
   wherein
   x is an integer of 1, 2, 3, 4, or 5, preferably of 1;
   y is an integer of 1, 2, 3, 4, or 5, preferably of 1;
   z is an integer of 1, 2, 3, 4, or 5, preferably of 1;
   k is an integer of 1, 2, 3, 4, or 5, preferably of 1; and
   n is an integer of 1 to 50, i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, preferably of 1, 2, 3, 4, 5, 6, 7, 8, 9, or 10.

Within the above preferred arrangements, “A” is an adapter within the above-defined meaning; “(c-b)” is used to indicate an embodiment according to the second aspect of the invention discussed in more detail below, wherein module (c) and -module (b) are comprised within one molecule; “-” indicates a covalent bond; and “---” indicates a non-covalent bond; the linker “L” in each instance can have, independently, all of above and below outlined meanings, i.e., a conjugate of the invention can comprise different types of linker molecules; and the linker “LB” is a linker as outlined above and below, which further comprises a branch point. The linker is preferably a carbohydrate chain of 1 to 40 carbon atoms in a linear or branched arrangement. It may additionally comprise between 1 to 15 heteroatoms, preferably oxygen, disulfide bonds, peptide bonds, and/or between 1 to 4 cycloalkyl, heterocycloalkyl, aromatic and/or heteroaromatic rings. Preferred examples of such linkers are provided in the Example section and in the figures. The linker may also be a chain of 1 to 20 amino acids, which are preferably linked by peptide bonds. Preferred amino acids comprised in the linker are small amino acids, which are preferably selected from the group consisting of Gly, Ala and Ser. It is understood that a wide variety of chemical bonds can be used to connect the linker with the respective module (a), (b), (c) and/or (d) as the case may b, preferably the bonds are formed by the reaction according to the general reaction schemes outlined below. Particularly preferred bonds are peptide or disulfide bonds. If two elements are to be connected by disulfide bonds, it is preferred that cysteine residues are located at the terminus of the respective module, linker or linker branch point to be connected. The branch point of the linker “LB” may be arranged at any position within the linker, e.g. at one of its ends or in the middle. Preferred branch points are side chains of amino acids, which are functionalized to allow coupling.

The term “cleavage site” in the context of the present invention means a specific amino acid sequence (e.g. a specific sequence within the amino acid sequence of the peptide linker molecule) or a specific chemical bond [e.g. a disulfide bond (S—S)] within the conjugate that is cleavable, e.g. via chemical cleavage or via cleavage by an enzyme, for example via a protease or peptidase that recognizes the specific sequence or via an enzyme which recognizes the specific chemical bond.

Wherein the linker molecule of the conjugate of the present invention comprises both a branch point and a cleavage site, it is preferred that the cleavage site is located upstream, e.g., 3′, of the branch point.

The presence of a cleavage site in the linker molecule connecting the at least one module (a), the at least one module (b), the at least one module (c), and/or the at least one compound (d) that may be arranged in any order, combination, or stoichiometry, of the conjugate of the present invention enables the separation of one or more of the modules and/or the at least one compound (d) during delivery of the compound (d) into a cell, e.g. after cellular uptake, after targeting the endoplasmic reticulum (ER), after delivery to the cytosol, or after delivery to the nucleus. Preferably, a conjugate of the present invention comprises at least 1, 2, 3, 4, 5, 6, 7, 8, 9, or 10 cleavage sites. More preferably, the conjugate comprises at least 1, 2, 3, 4, or 5 cleavage sites. Even more preferably, the conjugate comprises 1, 2, 3, 4, or 5 cleavage sites.

Preferably, a conjugate of the present invention comprises a cleavage site that is recognized by an enzyme, wherein the enzyme cleaves the conjugate at the cleavage site. The conjugate can be prepared with a cleavage site that is preferably recognized and cleaved by an enzyme that is located and active in a particular compartment or organelle of a cell or in the cell's cytosol. In a preferred embodiment, the conjugate comprises a cleavage site that is recognized and cleaved by an enzyme that is located and active in a target cell's endosome, a trans-Golgi network, Golgi, ER, cytosol, or nucleus. In another preferred embodiment, the conjugate comprises at least 2 cleavage sites, wherein each cleavage site is recognized and cleaved by at least 2 different enzymes, wherein the at least 2 different enzymes are each located and active in a different compartment, organelle or cytosol of a target cell.

In a specific embodiment, a conjugate of the present invention comprises a cleavage site that is recognized and cleaved by an endosomal enzyme, wherein the endosomal enzyme is preferably located and active in an early/recycling endosome. Preferably, the cleavage site is recognized and cleaved by furin, CHMP1A, ECE1, STAMBP, USP10, USP6, ZFYVE9, or the like.

In a specific embodiment, a conjugate of the present invention comprises a cleavage site that is recognized and cleaved by a trans-Golgi network enzyme. Preferably, the cleavage site is recognized and cleaved by furin and the like.

In a specific embodiment, a conjugate of the present invention comprises a cleavage site that is recognized and cleaved by a Golgi enzyme. Preferably, the cleavage site is recognized and cleaved by ADAM10, BACE1, CAPN8, CTSC, ECE2, MBTPS1, NCSTN, PCSK1, PCSK6, PCSK7, PSEN1, PSEN2, RHBDF1, Site-1 protease (S1P), Site-2 protease (S2P), SPPL2B, ZMPSTE24, or the like. In a particularly preferred embodiment, the cleavage site is recognized and cleaved by a Golgi-specific enzyme ECE2, PCSK7, SPPL2B, or the like.

In a specific embodiment, a conjugate of the present invention comprises a cleavage site that is recognized and cleaved by an ER enzyme. Preferably, the cleavage site is recognized and cleaved by a protein from the protein disulfide isomerase (PDI) family, BACE1, BACE2, CASP7, CTSA, CTSC, CTSH, CTSZ, cysteine protease ER-60, DPP4, ERAP2, ERMP1, HTRA2, KLK6, MBTPS1, NCLN, NCSTN, PCSK, PRSS50, RCE1, SPCS, TMPRSS3, ZMPSTE24, or the like.

In a specific embodiment, a conjugate of the present invention comprises a cleavage site that is recognized and cleaved by a cytosolic enzyme. Preferably, the cleavage site is recognized and cleaved by calpain or the like.

In a specific embodiment, a conjugate of the present invention comprises a cleavage site that is recognized and cleaved by a nuclear enzyme. Preferably, the cleavage site is recognized and cleaved by CAPN7, CASP1, CASP2, CASP3, CASP6, CASP7, CASP8, CASP14, GZMB, LONP_{—}2, PITRM1, PSMA1, PSMB1, PSMC1, PSME3, SENP_{—}1 or the like.

In a preferred embodiment, the cleavage site is positioned in the conjugate such that, when cleaved by the enzyme, the at least one module (a) of the conjugate is released from the conjugate. In this embodiment, the cleavage site is preferably positioned between module (a) and module (c) or module (b), or between module (a) and compound (d). Preferably, the cleavage site that releases module (a) from the conjugate is recognized and cleaved by an enzyme that is located and active in an endosome, the trans-Golgi network, the Golgi, the ER, the cytosol, or the nucleus of a target cell. More preferably, the cleavage site that releases module (a) from the conjugate is recognized and cleaved by an endosomal enzyme, a trans-Golgi network enzyme, a Golgi enzyme, an ER enzyme, a cytosolic enzyme, or a nuclear enzyme.

In another preferred embodiment, the cleavage site is positioned in the conjugate such that, when cleaved by the enzyme, the at least one module (b) of the conjugate is released from the conjugate. In this embodiment, the cleavage site is preferably positioned between module (b) and module (a) or module (c), or between module (b) and compound (d). Preferably, the cleavage site that releases module (b) from the conjugate is recognized and cleaved by an enzyme that is located and active in the ER, the cytosol, or the nucleus (e.g., calpain, a PDI family protein, BACE1, BACE2, CAPN7, CASP1, CASP2, CASP3, CASP6, CASP7, CASP8, CASP14, CTSA, CTSC, CTSH, CTSZ, DPP4, cysteine protease ER-60, ERAP2, ERMP1, GZMB, HTRA2, KLK6, LONP_{—}2, MBTPS1, NCLN, NCSTN, PCSK, PITRM1, PSMA1, PSMB1, PSMC1, PSME3, PRSS50, RCE1, SENP_{—}1, SPCS, TMPRSS3, ZMPSTE24, and the like). Preferably, the enzyme that is active in the ER, the cytosol, and/or the nucleus does not cleave off module (b) from the conjugate until the conjugate reaches the ER, the cytosol or the nucleus. More preferably, the cleavage site that releases module (b) from the conjugate is recognized and cleaved by an enzyme that is located and active in the ER, cytosol and/or nucleus but is not located or active in any of the cell compartments or organelles through which the conjugate of the present invention travels before reaching the ER, cytosol or nucleus. Even more preferably, the cleavage site that releases module (b) from the conjugate is recognized and cleaved by an enzyme that is located and active solely in the ER, the cytosol, and/or the nucleus.

In a specific embodiment, a conjugate of the present invention comprises a cleavage site within a peptide linker that is recognized and cleaved by an enzyme, wherein the enzyme is located and active in the ER, cytosol and/or nucleus but is not located or active in any of the cell compartments or organelles (e.g., endosomes, the Golgi, etc.) through which the conjugate of the present invention travels before reaching the ER, cytosol or nucleus (i.e., upstream of the ER, cytosol, or nucleus). Preferably, the cleavage site is recognized and cleaved by CASP7, CTSA, CTSH, CTSZ, ER-60, HTRA2, KLK6, NCLN, a PDI family protein, PRSS50, RCE1, TOR1A, and the like.

In another specific embodiment, a conjugate of the present invention comprises a cleavage site within a peptide linker that is recognized and cleaved by an enzyme, wherein the enzyme is located and active solely in the ER. Preferably, the cleavage site is recognized and cleaved by ER-60, ERMP1, a PDI family protein, SPCS1, TMPRSS3, or the like.

In another preferred embodiment, the cleavage site is positioned in the conjugate such that, when cleaved by the enzyme, the at least one compound (d) of the conjugate is released from the conjugate. In this embodiment, the cleavage site is preferably positioned between compound (d) and module (a), module (b) or module (c). When the compound (d) is desired to be delivered to the nucleus and the conjugate comprises a nuclear localization signal, the cleavage site is preferably positioned between compound (d) and the nuclear localization signal, and module (a), module (b) or module (c) such that, when cleaved by the enzyme, the at least one compound (d) and the nuclear localization signal are released from the conjugate. Preferably, the cleavage site that releases compound (d) or compound (d) and the nuclear localization signal from the conjugate is recognized and cleaved by an enzyme that is located and active in the cytosol or the nucleus.

In a preferred embodiment, the enzyme that is active in the cytosol or the nucleus does not cleave off compound (d) or compound (d) and the nuclear localization signal from the conjugate until the conjugate reaches the cytosol or the nucleus. More preferably, the cleavage site that releases compound (d) or compound (d) and the nuclear localization signal from the conjugate is recognized and cleaved by an enzyme that is located and active solely in the cytosol and/or the nucleus.

In a specific embodiment, a conjugate of the present invention comprises a cleavage site within a peptide linker that is recognized and cleaved by an enzyme, wherein the enzyme is located and active in the cytosol and/or nucleus but is not located or active in any of the cell compartments or organelles (e.g., endosomes, the trans Golgi network, the Golgi, the ER) through which the conjugate of the present invention travels before reaching the cytosol or nucleus (i.e., upstream of the cytosol or nucleus). Preferably, the cleavage site within a peptide linker is recognized and cleaved by calpain, ATG4A, CAPN10, CASP2, CASP3, CASP6, CASP9, GZMB, PREP, PREPL or the like.

In a preferred embodiment, a conjugate of the present invention comprises a cleavage site within a peptide linker that is recognized and cleaved by an enzyme, wherein the enzyme is located and active solely in the cytosol. Preferably, the cleavage site within a peptide linker is recognized and cleaved by calpain, PREPL or the like.

In another preferred embodiment, a conjugate of the present invention comprises a cleavage site within a peptide linker that is recognized and cleaved by an enzyme, wherein the enzyme is located and active solely in the nucleus. Preferably, the cleavage site within the peptide linker is recognized and cleaved by CAPN7, PITRM1, or the like.

In an alternative embodiment of the invention, the cleavage site within the conjugate is masked, such that the cleavage site is not available for cleavage until the conjugate reaches the intended compartment, organelle or cytosol in which cleavage at the cleavage site is desired. Masking of the cleavage site can be accomplished by a molecule that binds or interacts with the cleavage site within the conjugate, such that the masking molecule is released from the conjugate and the cleavage site is exposed when the conjugate reaches the intended compartment, organelle or cytosol in which cleavage of the conjugate is desired. Release of the masking molecule from the conjugate allows the cleavage enzyme to recognize and cleave the cleavage site and release the intended module, compound (d), or compound (d) and nuclear localization signal at the desired location within the cell. Alternatively, masking of a cleavage site within the conjugate of the invention may be due to the three-dimensional (3D) structure of the conjugate. In this alternative embodiment, a cleavage site is positioned within the conjugate such that it is internal (and therefore masked) within the 3D structure of the conjugate and is preferably made available for cleavage by removal of a portion of the conjugate (for example, when module (a) and/or module (b) is cleaved off from the conjugate, a cleavage site that is positioned between module (c) and compound (d) is no longer masked and is available for cleavage by its corresponding enzyme). Preferably, the masking molecule or the portion of the conjugate that is masking an internal cleavage site is released in the endosome, the TGN/Golgi Apparatus, the ER, the cytosol or the nucleus.

A preferred embodiment of the conjugate of the present invention comprises, for example, the following configuration: (a)ₓ, (d)ₙ, (c)_{z} and (b)_{y}, wherein (a)ₓ is covalently linked to (d)ₙ via a linker molecule comprising a cleavage site, (d)ₙ is covalently linked to (c)_{z} via a linker molecule comprising a different cleavage site and (c)_{z} is covalently linked to (b)_{y} and wherein x is an integer of 1, n is an integer of 1, 2, 3, 4, 5, 6, 7, 8, 9, or 10, z is an integer of 1 and y is an integer of 1. Thus, via the cleavage site between module (a) and module (d), it is possible to separate module (a) from the compound (d) and from the modules (c) and (b), e.g. after cellular uptake of the conjugate. As module (a) mediates cell targeting and facilitates cellular uptake, its function is no longer necessary after cell entry and thus, the presence of module (a) is no longer needed. It is further possible to separate compound (d) from the modules (b) and (c) via the cleavage site between compound (d) and module (c), e.g. after transfer to the cytosol.

In a preferred embodiment of the present invention, it is preferred to add a furin cleavage site within a peptide linker molecule, preferably within a peptide linker molecule that covalently links module (a) to compound (d) and modules (c) or (b) in order to separate module (a) from the compound (d) and from modules (c) and/or (b) after uptake into the cell and/or upon reaching the Golgi apparatus. The minimal furin cleavage site is Arg-X-X-Arg (SEQ ID NO: 105). However, the furin enzyme prefers the site Arg-X-(Lys/Arg)-Arg (SEQ ID NO: 106). Furin is the major processing enzyme of the secretory pathway and is localized in the trans-golgi network. It cleaves proteins or peptides and, thus, also peptide linkers, carrying an Arg-X-X-Arg (SEQ ID NO: 105) or Arg-X-(Lys/Arg)-Arg (SEQ ID NO: 106) sequence. As a result, furin will cleave the peptide linker at the furin cleavage site between module (a) and compound (d) and modules (c) or (b), during transport of the conjugate to the ER via the TGN/Golgi Apparatus and thus, separate the module (a) from compound (d) and from the modules (c) and/or (b). It is preferred to add a calpain cleavage site within the peptide linker molecule, preferably within the peptide linker molecule that covalently links compound (d) to modules (c) or (b) in order to separate compound (d) from modules (c) and/or (b) after transfer to the cytosol. The peptide TPLKSPPPSPR (SEQ ID NO: 107) can act as a calpain cleavage site [24].

In another preferred embodiment, a conjugate of the present invention may alternatively or additionally comprise a calpain cleavage site. Suitable cleavage sites occur commonly in various proteins and are known in the art. Preferred calpain cleavage sites are those present in the following proteins: ABP, Actin, Annexin I, Arrestin, Calpain 30K, Alpain 80K, CaMK IV, CaM-PDE1A2, Caspase-9, c-Fos, c-Jun, Connexin50, Beta-Crystallin A3, dystrophin, EGFR, GluR-1, a-Hemoglobin, b-Hemoglobin, Histone H2A, Histone H₂B, Histone H3.2, HMG-CoA reductase, Integrin beta 2, Integrin beta 3, Interleukin-1a, Interleukin-1a, MAP2c, MBP, Merlin, Phosphorylase kinase g, MIP, Myosin-V (brain), NKEF-B, NMDAR 2A, p35, p53, pADPRT, Phospholipase C-beta1, PKC-alpha, PKC-beta, PKC-gamma, PMCA-2, RyR1, Spectrin all, Spectrin b, Talin, Tau Tyrosine 3-hydroxylase, Vimentin and von Willebrand factor.

One of skill in the art can easily use another cleavage site(s) in place of or in addition to the cleavage sites recited herein. Cleavage recognition sequences for other enzymes are available and accessible to anyone skilled in the art.

Preferably, the compound of a conjugate of the present invention is covalently linked to the branch point, preferably via an amide-linkage to the lysine side chain, via a disulfide-linkage to the cysteine side chain or via an unnatural amino acid containing an aminooxy moiety on the side chain.

Thus, in a preferred embodiment of a conjugate according to the present invention, the modules and the compound (d) are linked to each other in the following arrangement, wherein module (a) is covalently linked to module (c) via a peptide linker molecule which comprises a cysteine side chain as branch point and a cleavage site upstream of the branch point, module (c) is covalently linked to module (b), and compound (d) is covalently linked via a disulfide-linkage to the cysteine side chain [for example, see FIG. 3(A)].

In another preferred embodiment of the conjugate according to the present invention, the modules and the compound are linked to each other in the following arrangement, wherein module (a) is covalently linked to module (c) via a peptide linker molecule which comprises a cysteine side chain as branch point and a cleavage site upstream of the branch point, module (c) is covalently linked to module (b) via a peptide linker molecule, and compound (d) is covalently linked via a disulfide-linkage to the cysteine side chain of the branch point [for example, see FIG. 3(B)].

The cleavage site in the peptide linker molecule connecting module (a) and module (c) enables the separation of module (a), e.g., after cell entry, from the modules (c) and (b). As the cleavage site is located upstream of the branch point of the peptide linker to which the compound (d) is covalently linked, compound (d) and modules (c) and (b) can be separated from module (a).

In another preferred embodiment, compound (d) is linked via an enzymatic cleavage site instead of the disulfide-linkage to the cysteine side chain [for example, see FIG. 3(C)]. Preferably, module (a) is cleaved off of the conjugate in the endosome or TGN, whereby making module (b) available for cellular receptors or other cellular proteins that bind to cellular receptors and then facilitate further transport to the ER. In a preferred embodiment, a furin (active in the endosome and TGN) cleavage site or another proprotein convertase cleavage site may be designed in the peptide linker molecules of the present invention to cleave off a module(s) that is no longer required for further transport within the cell. Such cleavage could occur in any cell organelle (e.g. endosome, TGN, Golgi, etc.) and one of ordinary skill in the art is able to synthesize a peptide linker molecule comprising a desired cleavage site using standard methods and without undue experimentation.

Preferably, the compound (d) of a conjugate of the present invention is non-covalently linked to the branch point via an ionic linkage or via a hydrophobic linkage to DRBD or a variant thereof that is covalently linked via a disulfide linkage to the cysteine side chain.

Thus, in a preferred embodiment of a conjugate according to the present invention, the at least one module (a), the at least one module (b), the at least module (c) and the at least one compound (d) are linked to each other in the following arrangements, wherein the at least one module (a) is covalently linked to the at least one module (c) via a peptide linker molecule which comprises a cysteine side chain as a branch point and a cleavage site upstream of the branch point, the at least one module (c) is covalently linked to the at least one module (b) and the at least one compound (d) is non-covalently linked to the branch point via an ionic (electrostatic) linkage to DRBD that is covalently linked via a disulfide-linkage to the cysteine side chain [for example, see FIG. 3(D)].

In another preferred embodiment, at least two (2) compounds (d) are non-covalently linked to the branch point via an ionic linkage to the DRBD that is covalently linked via the disulfide-linkage to the cysteine side chain.

In another preferred embodiment of the conjugate according to the present invention, for example, the modules and the compound are linked to each other in the following arrangement or combination, wherein module (a) is covalently linked to module (c) via a peptide linker molecule which comprises a cysteine side chain as branch point and a cleavage site upstream of the branch point, module (c) is covalently linked to module (b) via a peptide linker molecule and compound (d) is non-covalently linked to the branch point via an ionic linkage to DRBD that is covalently linked via a disulfide-linkage to the cysteine side chain [for example, see FIG. 3(E)].

It shall be understood that the conjugates described in FIGS. 1 (A) to (D), FIGS. 2 (A) and (B), FIGS. 3 (A) to (E), FIG. 4, FIG. 5, FIGS. 6 (A) and (B), FIG. 7, FIG. 8, FIG. 9, FIGS. 10 (A) and (B), FIG. 11, FIG. 12, FIG. 13, FIG. 14, FIG. 22, FIG. 23, FIG. 24, FIG. 25, FIG. 26, FIG. 27, FIG. 28, and FIG. 29 represent only a small portion of the possible configurations of a conjugate of the present invention. One of skill in the art can make conjugates of other configurations without undue experimentation, and these conjugates are also encompassed within the scope of the present invention.

The conjugate of the present invention preferably comprises modules that are of endogenous origin in order to minimize the risk of unexpected immune reactions. Modules from exogenous sources may also be used within a conjugate of the present invention. If a module(s) from an exogenous source is used within a conjugate of the present invention, it is preferred that the exogenous module carries minimal risk of toxicity, or other unwanted activities such as immune activation, or oncogenicity.

The conjugate of the present invention comprises at least one module that mediates cell targeting and facilitates cellular uptake, designated as module (a), and is preferably of human origin.

Basically any molecule or structure that has high affinity binding to one or more than one molecule or structure on the surface of a target cell is suitable as module (a), and preferably triggers internalization into vesicular compartments capable of undergoing retrograde transport. Alternatively, module (a) can provide this target cell uptake functionality indirectly by binding to a molecule outside the target cells (i.e., in a pre-incubation before use, in the cell culture media or in an organism's blood, spinal fluid, interstitial fluid, etc., and defined herein as a “indirect targeting adapter molecule”), wherein the target cells directly recognize the indirect targeting adapter molecule, and wherein the indirect targeting adapter molecule preferably triggers internalization into vesicular compartments capable of undergoing retrograde transport.

In a preferred embodiment, a bispecific antibody (e.g., diabody or single-chain antibody) is used to bind both module (a) of the conjugate and a cell surface receptor on a desired target cell. Briefly, the bispecific antibody is pre-incubated with a conjugate comprising a module (a) that is recognized by the bispecific antibody before exposure or administration of the conjugate to a target cell. Upon exposure or administration to the target cell, the bispecific antibody-conjugate complex binds to the cell surface receptor that is recognized by the bispecific antibody. As a result of binding to the cell surface receptor, the bispecific antibody-conjugate complex preferably triggers internalization into a vesicular compartment from which retrograde transport can be initiated. In another embodiment, module (a) comprises an antibody (immunoglobulin, Ig) binding domain that is able to bind to an antibody that binds to a cell surface receptor on a desired target cell, thereby indirectly targeting the conjugate of the present invention to a cell of interest. In another preferred embodiment, module (a) comprises a biotin acceptor peptide that is able to bind to a biotinylated ligand that binds to a cell surface receptor on a desired target cell to indirectly target the conjugate of the present invention to the cell of interest.

Thus, the present invention provides a flexible platform for cell targeting since any ligand or binding particle that is able to enter a cell using endocytosis, and preferably triggers internalization into vesicular compartments capable of undergoing retrograde transport, can be exploited to target the conjugates of the present invention to a desired cell. Indeed, such targeting approaches are commonly used for targeting viral vectors and are well described in the literature (see for example, [25]). In addition, this indirect targeting approach is advantageous for the development of reagents for use with a delivery system or conjugate of the present invention, or kits comprising the same. Thus, one of skill in the art will be able to recognize and use different combinations of a module (a) and an indirect targeting adapter molecule to indirectly target conjugates encompassed by the present invention to a cell of interest, without undue experimentation.

In a particularly preferred embodiment, a conjugate of the present invention comprises a module (a) that either directly or indirectly confers a transcytosis functionality, whereby the conjugate can penetrate through or within a tissue, a tumor, an endothelial cell, and the like. Examples of molecules that may be used as module (a) for trancystosis functionality include but are not limited to albumin, orosomucoid, IgG, low density lipoprotein (LDL) cholesterol (not via LDL receptor), gonadotrophin, transferrin (not via transferrin receptor), melanotransferrin (p97; [26]), insulin, LDL, dIgA (dimeric immunoglobulin (Ig)A), vitamin B12, vitamin D, vitamin A, iron, HRP (horseradish peroxidase), ferritin, thyroglobulin, and the like (for a review, see [27]). Alternatively, one can use an antibody directed to albumin, orosomucoid, IgG, LDL cholesterol (not via LDL receptor), gonadotrophin, transferrin (not via transferrin receptor), melanotransferrin (p97), insulin, LDL, dIgA, vitamin B12, vitamin D, vitamin A, iron, HRP, ferritin, thyroglobulin, and the like, as a module (a) comprising a transcytosis functionality for use in a conjugate of the present invention.

All molecules, which are naturally taken up by any cell with high efficiency and fast kinetics can be used as module (a) or indirectly, to bind to module (a), provided that the molecule is internalized into or arrives in an intracellular membranous organelle. Such molecules preferably carry a low risk of eliciting an immune response or toxicity. Other molecules known to undergo cellular uptake, but which also carry certain secondary activities, such as an increased risk of immune stimulation may also be used as module (a).

Preferably, module (a), or the indirect targeting adapter molecule to which module (a) binds, of the conjugate of the present invention comprises a ligand of a cell surface marker that allows, causes and/or results in specific cell targeting and cellular uptake. Preferably, said ligand of a cell surface marker is a cell surface receptor ligand, an antibody, a sugar, a lipid or a nanoparticle, preferably of human origin.

It is particularly preferred that the cell surface receptor ligand is a ligand selected from the group consisting of a growth factor, a autocrine motility factor (AMF), a lipoprotein, a transferrin, a surface binding lectin, a galectin, a c-type lectin, a toxin, a Wnt related protein or peptide, an amyloid precursor protein (APP), an apolipoprotein A-V, a fragment thereof, and a variant thereof.

Preferably, the cell surface receptor ligand is a growth factor selected from the group consisting of EGF, VEGF, BMPs, FGF, G-CSF, GM-CSF, HGF, GDFs, IGFs, NGF, TGFs, PGF, and PDGF.

In a preferred embodiment, the cell surface receptor ligand is an Autocrine Motility Factor [AMF, also known as phosphoglucose isomerase (PGI)]. AMF or other peptides, proteins, and small molecules that bind to AMF receptors and trigger its internalization are preferred cell surface receptor ligands of the present invention. Preferably, an AMF peptide of use in the conjugates of the present invention comprises an amino acid sequence comprising SEQ ID NO: 108 (full length human AMF), or a fragment or variant thereof. In another embodiment, an AMF peptide of use in the conjugates of the present invention comprises an amino acid sequence comprising SEQ ID NO: 109 (full length mouse AMF), or a fragment or variant thereof. In another embodiment, an AMF peptide of use in the conjugates of the present invention comprises an amino acid sequence comprising SEQ ID NO: 311 (full length rabbit AMF), or a fragment or variant thereof.

In another preferred embodiment, the cell surface receptor ligand is a sulfatase-modifying factor (SUMF). SUMF or other peptides, proteins, and small molecules that bind to SUMF receptors and trigger its internalization are preferred cell surface receptor ligands of the present invention. Preferably, an SUMF peptide or protein of use in the conjugates of the present invention comprises an amino acid sequence comprising human SUMF1 protein (SEQ ID NO: 110; UniProtKB/Swiss-Prot Q8NBK3 [28]), or a fragment of variant thereof.

Preferably, the cell surface ligand is a lipoprotein selected from the group consisting of a high density liproprotein (HDL) receptor/scavenger receptor family lipoprotein, a low density lipoprotein (LDL) receptor family lipoprotein, and an apolipoprotein A-V (Nilsson et al., J Biol. Chem. 2008. 283(38):25920-7. Epub 2008 Jul. 3).

Preferably, the cell surface ligand is a transferrin receptor (TfR) binding peptide selected from the group consisting of THRPPMWSPVWP (SEQ ID NO: 111; [29] and U.S. Pat. No. 6,743,893), GHKVKRPKG (SEQ ID NO: 112; [30] and WO2003/050238), and HAIYPRH (SEQ ID NO: 113; [29]).

Preferably, the cell surface ligand is a lectin selected from the group consisting of a soluble lectin, a collectin, and an intelectin (ITLN).

Preferably, the cell surface ligand is a galectin selected from the group consisting of LGALS1, LGALS2, LGALS3, LGALS4, LGALS5, LGALS6, LGALS7, LGALS8, LGALS9, LGALS10, LGALS11, LGALS12, and LGALS13.

Preferably, the cell surface ligand is a toxin selected from the group consisting of a bacterial toxin and a plant toxin. In a preferred embodiment, module (a) of the conjugate of the present invention comprises or consists of a toxin protein or peptide selected from the group consisting of a toxin protein or peptide having reduced or no toxicity, an A/B type toxin protein or peptide having reduced or no toxicity, an A/B₅ type toxin protein or peptide having reduced or no toxicity, an A/B type toxin subunit having reduced or no toxicity, an A/B₅ type toxin subunit having reduced or no toxicity, an A/B type holo-toxin having reduced or no toxicity, an A/B₅ type holo-toxin having reduced or no toxicity, an A/B type toxin B subunit, an A/B₅ type toxin B-subunit, a non-toxic ricin holo-toxin, a non-toxic ricin holotoxin wherein in the ricin A subunit has an R180H mutation (SEQ ID NO: 1), a mutant ricin holotoxin with reduced or no toxicity, a ricin toxin B-subunit (RTB), a ricin toxin B-subunit peptide, a cholera toxin (CT) B-subunit (CTB), a cholera toxin B-subunit peptide, a non-toxic Shiga holo-toxin, a non-toxic Stx1a Shiga holo-toxin, a non-toxic Stx1b (VT1b) Shiga holo-toxin, a non-toxic Stx1c (VT1c) Shiga holo-toxin, a non-toxic Stx1d (VT1d) Shiga holo-toxin, a non-toxic Stx2a (VT2a) Shiga holo-toxin, a non-toxic Stx2b (VT2b) Shiga holo-toxin, a non-toxic Stx2c (VT2c) Shiga holo-toxin, a non-toxic Stx2d (VT2d) Shiga holo-toxin, a non-toxic Stx2e (VT2e) Shiga holo-toxin, a non-toxic Stx2f (VT2f) Shiga holo-toxin, a non-toxic Stx2g (VT2g) Shiga holo-toxin, a mutant Shiga holo-toxin having reduced or no toxicity, a mutant Stx1a Shiga holo-toxin having reduced or no toxicity, a mutant Stx1b (VT1b) Shiga holo-toxin having reduced or no toxicity, a mutant Stx1c (VT1c) Shiga holo-toxin having reduced or no toxicity, a mutant Stx1d (VT1d) Shiga holo-toxin having reduced or no toxicity, a mutant Stx2a (VT2a) Shiga holo-toxin having reduced or no toxicity, a mutant Stx2b (VT2b) Shiga holo-toxin having reduced or no toxicity, a mutant Stx2c (VT2c) Shiga holo-toxin having reduced or no toxicity, a mutant Stx2d (VT2d) Shiga holo-toxin having reduced or no toxicity, a mutant Stx2e (VT2e) Shiga holo-toxin having reduced or no toxicity, a mutant Stx2f (VT2f) Shiga holo-toxin having reduced or no toxicity, a mutant Stx2g (VT2g) Shiga holo-toxin having reduced or no toxicity, a Shiga toxin (ST) B-subunit (STB), an Stx1a Shiga toxin B-subunit, an Stx1b (VT1b) Shiga toxin B-subunit, an Stx1c (VT1c) Shiga toxin B-subunit, an Stx1d (VT1d) Shiga toxin B-subunit, an Stx2a (VT2a) Shiga toxin B-subunit, an Stx2b (VT2b) Shiga toxin B-subunit, an Stx2c (VT2c) Shiga toxin B-subunit, an Stx2d (VT2d) Shiga toxin B-subunit, an Stx2e (VT2e) Shiga toxin B-subunit, an Stx2f (VT2f) Shiga toxin B-subunit, an Stx2g (VT2g) Shiga toxin B-subunit, a Shiga toxin B-subunit peptide, an Stx1a Shiga toxin B-subunit peptide, an Stx1b (VT1b) Shiga toxin B-subunit peptide, an Stx1c (VT1c) Shiga toxin B-subunit peptide, an Stx1d (VT1d) Shiga toxin B-subunit peptide, an Stx2a (VT2a) Shiga toxin B-subunit peptide, an Stx2b (VT2b) Shiga toxin B-subunit peptide, an Stx2c (VT2c) Shiga toxin B-subunit peptide, an Stx2d (VT2d) Shiga toxin B-subunit peptide, an Stx2e (VT2e) Shiga toxin B-subunit peptide, an Stx2f (VT2f) Shiga toxin B-subunit peptide, an Stx2g (VT2g) Shiga toxin B-subunit peptide, an Escherichia coli heat labile enterotoxin (LT) B-subunit, an LT-IIa B-subunit, an LT-IIb B-subunit, an Abrin-a B-subunit, an Abrin-b B-subunit, an Abrin-c B-subunit, an Abrin-d B-subunit, a Pertussis B-subunit, a Modeccin B-subunit, a Volkensin B-subunit, a Viscumin B-subunit, a Pseudomonas exotoxin A Domain IA, an Escherichia coli subtilase cytotoxin B-subunit, a Tetanus toxin C-fragment, a hybrid AB toxin with reduced or no toxicity, a hybrid ricin-abrin toxin with reduced or no toxicity, a hybrid ricin A-subunit (RTA)-abrin B-subunit (AB-B) toxin with reduced or no toxicity, a hybrid abrin A-subunit (AB-A)-ricin B-subunit (RTB) with reduced or no toxicity, a hybrid AB₅ toxin with reduced or no toxicity, a hybrid LT-CT toxin with reduced or no toxicity, a hybrid A1(LT1)-A2(CT)-B5(CT) toxin with reduced or no toxicity, a hybrid SLT-ST toxin with reduced or no toxicity, and a hybrid A1(SLT)-A2(ST)-B5(ST) toxin with reduced or no toxicity. Preferably, the toxin protein or peptide for use in the conjugates of the invention lacks a signal peptide.

In a preferred embodiment, wherein one or more modules of the conjugate of the invention comprises a Shiga toxin protein, peptide, subunit, subunit peptide or holo-toxin, the Shiga toxin may be selected from type Stx1 or type Stx2. There are 4 subtypes of Stx1 Shiga toxins: Stx1a, which is the “true” Shiga toxin from Shigella dysenteriae or Shigella sonnei and the three closely related “Shiga-like toxin I” [(SLT-I) and also referred to as “Verotoxin” (VT)] Shiga subtypes Stx1b (VT1b), Stx1c (VT1c), and Stx1d (VT1d) from E. coli. There are 7 subtypes of Stx2 Shiga toxins, all of which are closely related “Shiga-like toxin II” (“SLT-II” or “VT2”) Shiga toxins from E. coli and are designated as Stx2a (VT2a), Stx2b (VT2b), Stx2c (VT2c), Stx2d (VT2d), Stx2e (VT2e), Stx2f (VT2f), and Stx2g (VT2g). Shiga toxin is an A/B toxin, meaning that a Shiga holo-toxin comprises an A subunit (comprising A and A2) and a B subunit.

Preferably, when a conjugate of the invention comprises one or more modules comprising, containing, or consisting of a Shiga toxin protein, peptide, subunit, subunit peptide or holo-toxin, the Shiga toxin comprises, consists essentially of or consists of at least one amino acid sequence selected from the group consisting of SEQ ID NO: 119 (Stx1a B subunit), SEQ ID NO: 120 (Stx1b B subunit, NCBI Ref. Seq.: NP_{—}288672.1), SEQ ID NO: 121 (Stx1c B subunit, GenBank: ABE02588.1), SEQ ID NO: 122 (Stx1d B subunit, GenBank: AA019476.1), SEQ ID NO: 123 (Stx2a B subunit, GenBank: AAG55588.1), SEQ ID NO: 124 (Stx2b B subunit, GenBank: BAB82993.1), SEQ ID NO: 125 (Stx2c B subunit, reference strain, GenBank: CAC05566.1), SEQ ID NO: 126 (Stx2c B subunit, sub type variant, NCBI Ref Seq.: YP_{—}003078595.1), SEQ ID NO: 127 (Stx2d B subunit, reference strain, GenBank: AAM88313.2), SEQ ID NO: 128 (Stx2d B subunit, variant strain 1, GenBank: AAN77056.1), SEQ ID NO: 129 (Stx2d B subunit, variant strain 2, GenBank: AAN77064.1), SEQ ID NO: 227 (Stx2d B subunit, variant strain 3, GenBank: ADV16384.1), SEQ ID NO: 228 (Stx2e B subunit, GenBank: AAQ63639.1), SEQ ID NO: 229 (Stx2f B subunit, reference strain, GenBank: CAC05561.1), SEQ ID NO: 230 (Stx2f B subunit, variant strain 1, GenBank: BAH86760.1), variant strain 2), SEQ ID NO: 231 (Stx2g B subunit, GenBank: ADN64240.1), SEQ ID NO: 157 (Stx1a A1 subunit peptide, GenBank: AAF28121.1), SEQ ID NO: 158 (Stx1b A1 subunit peptide, reference strain, Swiss-Prot: P08026.1), SEQ ID NO: 232 (Stx1b A1 subunit peptide, variant strain, NCBI Ref Seq.: NP 288673.1), SEQ ID NO: 233 (Stx1c A1 subunit peptide, GenBank: ABE02587.1), SEQ ID NO: 234 (Stx1d A1 subunit peptide, GenBank: AA019475.1), SEQ ID NO: 235 (Stx2a A1 subunit peptide, GenBank: AAG55587.1), SEQ ID NO: 236 (Stx2b A1 subunit peptide, Swiss-Prot: Q9S5J3), SEQ ID NO: 237 (Stx2c A1 subunit peptide, reference strain, GenBank: ADF56034.1), SEQ ID NO: 238 (Stx2c A1 subunit peptide, variant strain 1, GenBank: CCA65428.1), SEQ ID NO: 239 (Stx2c A1 subunit peptide, variant strain 2, GenBank: CCA65430.1), SEQ ID NO: 240 (Stx2d A1 subunit peptide, reference strain, GenBank: AAN77059.1), SEQ ID NO: 241 (Stx2d A1 subunit peptide, variant strain 1, GenBank: AAN77063.1), SEQ ID NO: 242 (Stx2d A1 subunit peptide, variant strain 2, GenBank: AAN77057.1), SEQ ID NO: 243 (Stx2d A1 subunit peptide, variant strain 3, GenBank: AAN77065.1), SEQ ID NO: 244 (Stx2d A1 subunit peptide, variant strain 4, GenBank: AAN77061.1), SEQ ID NO: 245 (Stx2d A1 subunit peptide, variant strain 5, GenBank: CAX45706.1), SEQ ID NO: 246 (Stx2e A1 subunit peptide, reference strain, GenBank: AAQ63638.1), SEQ ID NO: 247 (Stx2e A1 subunit peptide, variant strain 1, GenBank: CAX51710.1), SEQ ID NO: 248 (Stx2e A1 subunit peptide, variant strain 2, GenBank: CAX45724.1), SEQ ID NO: 249 (Stx2e A1 subunit peptide, variant strain 3, GenBank: CAX45714.1), SEQ ID NO: 250 (Stx2e A1 subunit peptide, variant strain 4, GenBank: CAX45702.1), SEQ ID NO: 251 (Stx2e A1 subunit peptide, variant strain 5, GenBank: CAX51714.1), SEQ ID NO: 252 (Stx2f A1 subunit peptide, reference strain, GenBank: CAC05560.1), SEQ ID NO: 253 (Stx2f A1 subunit peptide, variant strain, GenBank: BAH86759.1), SEQ ID NO: 254 (Stx2g A1 subunit peptide, reference strain, GenBank: ADN64239.1), SEQ ID NO: 255 (Stx2g A1 subunit peptide, variant strain 1, GenBank: ADN34746.1), SEQ ID NO: 256 (Stx2 A1 subunit peptide, GenBank: AAM22256.1), SEQ ID NO: 162 (Stx1a A subunit, GenBank: AAF28121.1), SEQ ID NO: 163 (Stx1b A subunit, reference strain, Swiss-Prot: P08026.1), SEQ ID NO: 164 (Stx1b A subunit, variant strain, NCBI Ref. Seq.: NP_{—}288673.1), SEQ ID NO: 165 (Stx1c A subunit, GenBank: ABE02587.1), SEQ ID NO: 166 (Stx1d A subunit, GenBank: AA019475.1), SEQ ID NO: 257 (Stx2a A subunit, GenBank: AAG55587.1), SEQ ID NO: 258 (Stx2b A subunit, Swiss-Prot: Q9S5J3), SEQ ID NO: 259 (Stx2c A subunit, reference strain, GenBank: ADF56034.1), SEQ ID NO: 260 (Stx2c A subunit, variant strain 1, GenBank: CCA65428.1), SEQ ID NO: 261 (Stx2c A subunit, variant strain 2, GenBank: CCA65430.1), SEQ ID NO: 262 (Stx2d A subunit, reference strain, GenBank: AAN77059.1), SEQ ID NO: 263 (Stx2d A subunit, variant strain 1, GenBank: AAN77063.1), SEQ ID NO: 264 (Stx2d A subunit, variant strain 2, GenBank: AAN77057.1), SEQ ID NO: 265 (Stx2d A subunit, variant strain 3, GenBank: AAN77065.1), SEQ ID NO: 266 (Stx2d A subunit, variant strain 4, GenBank: AAN77061.1), SEQ ID NO: 267 (Stx2d A subunit, variant strain 5, GenBank: CAX45706.1), SEQ ID NO: 268 (Stx2e A subunit, reference strain, GenBank: AAQ63638.1), SEQ ID NO: 269 (Stx2e A subunit, variant strain 1, GenBank: CAX51710.1), SEQ ID NO: 270 (Stx2e A subunit, variant strain 2, GenBank: CAX45724.1), SEQ ID NO: 271 (Stx2e A subunit, variant strain 3, GenBank: CAX45714.1), SEQ ID NO: 272 (Stx2e A subunit, variant strain 4, GenBank: CAX45702.1), SEQ ID NO: 273 (Stx2e A subunit, variant strain 5, GenBank: CAX51714.1), SEQ ID NO: 274 (Stx2f A subunit, reference strain, GenBank: CAC05560.1), SEQ ID NO: 275 (Stx2f A subunit, variant strain 1, GenBank: BAH86759.1), SEQ ID NO: 276 (Stx2f A subunit, variant strain 2, GenBank: BAE79483.1), SEQ ID NO: 277 (Stx2g A subunit, reference strain, GenBank: ADN64239.1), SEQ ID NO: 278 (Stx2g A subunit, variant strain 1, GenBank: ADN34746.1), and SEQ ID NO: 279 (Stx2 A subunit, GenBank: AAM22256.1).

In a particular embodiment, a conjugate of the present invention comprises a module (a) comprising, essentially consisting of, or consisting of a holo-toxin, a toxin, or a hybrid protein or peptide, wherein the holo-toxin, toxin, or hybrid protein or peptide is non-toxic or has reduced toxicity. In a preferred embodiment, a non-toxic or reduced toxicity holo-toxin, toxin, or hybrid protein or peptide comprises an amino acid deletion, substitution, or insertion that results in a mutated holo-toxin, mutated toxin, or mutated hybrid protein or peptide having reduced or no toxicity compared to the wild-type holo-toxin, toxin, or hybrid protein or peptide.

In a preferred embodiment, module (a) comprises or consists of a holo-toxin or hybrid toxin comprising a non-toxic or reduced toxicity protein or peptide of ricin toxin A1-subunit (SEQ ID NO: 1; ricin toxin A comprising an R180H substitution). In another preferred embodiment, module (a) comprises or consists of a holo-toxin or hybrid toxin comprising a mutated ricin toxin A1-subunit having reduced or no toxicity, wherein the mutated ricin toxin A1-subunit comprises a G247W substitution, an S250P substitution, a G247Q substitution, a W246R substitution, an E212D substitution, an E212K substitution, an 1287R substitution (Frankel et al., Mol Cell Biol. 1989. 9(2):415-20), an R215Q substitution, an E212Q substitution, a Y115S substitution, a Y158S substitution (Kim and Robertus, Protein Eng. 1992 December; 5(8):775-9), a deletion of amino acids 110-115 (DVTNAY; Ricin-Δ110-115; May et al., EMBO J. 1989. 8(1):301-8), or a Y115A/V111M double substitution (RiVax; Vitetta et al., Proc Natl Acad Sci USA. 2006 Feb. 14; 103(7):2268-73. Epub 2006 Feb. 3), and wherein the numerical position of the mutated ricin toxin A1-subunit's amino acid substitution or deletion is based upon the Uniprot sequence P02879 that comprises the full length ricin amino acid sequence, including the signal peptide. While the reference sequence used here (i.e., Uniprot sequence P02879) to identify the location of the mutations in the ricin toxin A-subunit comprises a signal peptide (amino acids 1-25 of Uniprot P02879), the mutated ricin toxin A1-subunit protein or peptide for use in a holo-toxin or hybrid toxin module (a) of the invention preferably lacks this signal peptide.

In another preferred embodiment, module (a) comprises or consists of a holo-toxin or hybrid toxin comprising a non-toxic or reduced toxicity protein or peptide of Pseudomonas exotoxin A (http://www.uniprot.org/uniprot/P11439>sp|P11439|26-638 lacking the signal peptide sequence). Preferably, module (a) comprises or consists of a non-toxic or reduced toxicity holo-toxin or hybrid toxin comprising or consisting of an amino acid sequence selected from the group consisting of amino acids 1-613 of SEQ ID NO: 114 (holo-toxin Pseudomonas exotoxin A lacking a signal peptide) and a mutated Pseudomonas exotoxin A having reduced or no toxicity, wherein the mutated Pseudomonas exotoxin A comprises a D599C substitution or an E553D substitution [see Benhar et al., J Biol. Chem. 1994. 269(18):13398-404, and Douglas and Collier, J. Bacteriol. 1987. 169(11):4967-71, respectively and P11439 (TOXA_PSEAE)]. Preferably, a non-toxic or reduced toxicity Pseudomonas exotoxin A protein or peptide for use in a holo-toxin or hybrid toxin module (a) of the invention preferably lacks a signal peptide.

In yet another preferred embodiment, module (a) comprises, consists essentially, or consists of a toxin protein or peptide selected from the group consisting of a ricin toxin B-subunit protein or peptide comprising or consisting of an amino acid sequence according to SEQ ID NO: 115 or SEQ ID NO: 116, or a recombinantly produced ricin toxin B-subunit as described in WO2008/157263; a cholera toxin B-subunit protein or peptide comprising or consisting of an amino acid sequence according to SEQ ID NO: 117 or SEQ ID NO: 118; a Shiga toxin (Stx) B-subunit protein or peptide comprising or consisting of an amino acid sequence according to SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 227, SEQ ID NO: 228, SEQ ID NO: 229, SEQ ID NO: 230, SEQ ID NO: 231, fragment thereof, or variant thereof; an LT-B B-subunit protein or peptide comprising or consisting of an amino acid sequence according to SEQ ID NO: 130 or SEQ ID NO: 131; an LT-Ha B-subunit protein or peptide comprising or consisting of an amino acid sequence according to SEQ ID NO: 132; an LT-IIb B-subunit protein or peptide comprising or consisting of an amino acid sequence according to SEQ ID NO: 133; an abrin toxin B-subunit protein or peptide comprising or consisting of an amino acid sequence according to SEQ ID NO: 134, amino acids 262-528 of SEQ ID NO: 135 (Abrin a toxin), amino acids 261-527 of SEQ ID NO: 136 (Abrin b toxin), amino acids 296-562 of SEQ ID NO: 137 (Abrin c toxin), or amino acids 262-528 of SEQ ID NO: 138 (Abrin d toxin); a pertussis toxin B-subunit comprising or consisting of an S2 protein, an S3 protein, two S4 proteins, and an S5 protein, wherein the S2 protein comprises an amino acid sequence comprising SEQ ID NO: 139 (Pertussis toxin subunit 2 (PTX S2); http://www.uniprot.org/uniprot/P04978), the S3 protein comprises an amino acid sequence comprising SEQ ID NO: 140 (Pertussis toxin subunit 3 (PTX S3); http://www.uniprot.org/uniprot/P04979), each of the two S4 proteins comprise an amino acid sequence comprising SEQ ID NO: 141 (Pertussis toxin subunit 4 (PTX S4); http://www.uniprot.org/uniprot/P0A3R5), and the S5 protein comprises an amino acid sequence comprising SEQ ID NO: 142 (Pertussis toxin subunit 5 (PTX S5); http://www.uniprot.org/uniprot/P04981); an E. coli subtilase cytotoxin B-subunit comprising or consisting of an amino acid sequence of SEQ ID NO: 143, SEQ ID NO: 144, or SEQ ID NO: 145; a volkensin toxin B-subunit comprising or consisting of an amino acid sequence comprising SEQ ID NO: 146 (Chambery et al., Eur J. Biochem. 2004. 271(1):108-17); a viscumin B-subunit comprising or consisting of an amino acid sequence comprising SEQ ID NO: 147 (http://www.uniprot.org/uniprot/P81446); a tetanus toxin C-fragment comprising or consisting of an amino acid sequence comprising SEQ ID NO: 148 and SEQ ID NO: 149; a Pseudomonas exotoxin A Domain IA comprising or consisting of amino acids 1-252 of SEQ ID NO: 114, a fragment thereof, and a variant thereof.

Preferably, a conjugate of the present invention comprises at least one module (a) that comprises an Escherichia coli subtilase cytotoxin (SubAB). SubAB exerts its effect in the ER, a characteristic that can be exploited to deliver the DARE payload, i.e., at least one compound (d), into the ER. A combination of a SubAB and a second module (c), e.g. Cox2 or Sgk1, will facilitate transport to the cytosol. The amino acid sequence of E. coli SubAB toxin is published (see http://www.uniprot.org/uniprot/?query=subtilase+cytotoxin&sort=score, B subunit: http://www.ncbi.nlm.nih.gov/protein/ABI06311.1, and A subunit: http://www.ncbi.nlm.nih.gov/protein/ABI06310.1).

Preferably, a conjugate of the present invention comprises at least one module (a) that comprises a tetanus toxin C-fragment. The tetanus toxin C-fragment is the C-terminal fragment of the heavy chain (fragment C or HC) and is similar in function to the B-subunits of other toxins. The tetanus toxin C-fragment facilitates binding to a cell and retrograde transport in neurons.

In another embodiment, a module (a) or a [module (a)+module (b)+module (c)] protein or peptide of the conjugate of the present invention comprises or consists of a reduced toxicity or non-toxic hybrid toxin protein or peptide. Preferably, the reduced toxicity or non-toxic hybrid toxin protein or peptide comprises a reduced toxicity or non-toxic A-subunit and a B-subunit, each of which are from at least two different toxins. In the case of AB toxins, a reduced toxicity or non-toxic A-subunit from one AB toxin is combined with a B-subunit from a second AB toxin to result in a reduced toxicity or non-toxic hybrid AB toxin protein or peptide. Preferable AB toxins of use in the conjugates of the present invention include ricin, abrin a, abrin b, abrin c, abrin d, modeccin, viscumin, volkensin, and the like. Alternatively, a reduced toxicity or non-toxic A-subunit from one AB₅ toxin is combined with a B₅-subunit from a second AB₅ toxin to result in a reduced toxicity or non-toxic hybrid AB₅ toxin protein or peptide. Preferable AB₅ toxins of use in the conjugates of the present invention include cholera toxin, Shiga toxin, Shiga-like toxins, E. coli heat-labile enterotoxins, pertussis toxin, and the like. Preferably, the hybrid AB₅ toxin protein or peptide comprises a non-toxic A2-subunit and B-subunit pentamer (B₅) from one AB₅ toxin and a reduced toxicity or non-toxic A1-subunit from a second AB₅ toxin, e.g., an A1(LTI) having reduced or no toxicity+an A2(CTx)+B5(CTx) hybrid toxin protein. Preferably, the reduced toxicity or non-toxic A1-subunit of the hybrid toxin protein or peptide comprises a mutation that results in reduced or no toxicity, e.g., a mutated A1(LTI) having reduced or no toxicity+an A2(CTx)+B5(CTx) hybrid toxin protein.

Thus, a particularly preferred module (a) or [module (a)+module (b)+module (c)] protein or peptide of the conjugate of the present invention comprises or consists of a hybrid toxin with reduced or no toxicity, wherein the hybrid toxin comprises a mutated A-subunit of a first AB toxin and a B-subunit of a second and different AB toxin, wherein the first AB toxin and the second and different AB toxin are each selected from the group consisting of a ricin, an abrin a, an abrin b, an abrin c, an abrin d, a modeccin, a viscumin, and a volkensin toxin. Preferably, the hybrid AB toxin with reduced or no toxicity comprises: a mutated A-subunit of a ricin toxin and a B-subunit of an abrin a, an abrin b, an abrin c, an abrin d, a modeccin, a viscumin, or a volkensin toxin; a mutated A-subunit of an abrin a toxin and a B-subunit of a ricin, an abrin b, an abrin c, an abrin d, a modeccin, a viscumin, or a volkensin toxin; a mutated A-subunit of an abrin b toxin and a B-subunit of a ricin, an abrin a, an abrin c, an abrin d, a modeccin, a viscumin, or a volkensin toxin; a mutated A-subunit of an abrin c toxin and a B-subunit of a ricin, an abrin a, an abrin b, an abrin d, a modeccin, a viscumin, or a volkensin toxin; a mutated A-subunit of an abrin d toxin and a B-subunit of a ricin, an abrin a, an abrin b, an abrin c, a modeccin, a viscumin, or a volkensin toxin; a mutated A-subunit of a modeccin toxin and a B-subunit of a ricin, an abrin a, an abrin b, an abrin c, an abrin d, a viscumin, or a volkensin toxin; a mutated A-subunit of a viscumin toxin and a B-subunit of a ricin, an abrin a, an abrin b, an abrin c, an abrin d, a modeccin, or a volkensin toxin; or a mutated A-subunit of a volkensin toxin and a B-subunit of a ricin, an abrin a, an abrin b, an abrin c, an abrin d, a modeccin, a viscumin, or a volkensin toxin. Exemplary but non-limiting embodiments of a hybrid AB toxin include a hybrid ricin A-subunit (RTA)-abrin a B-subunit (ABa-B) toxin with reduced or no toxicity and a hybrid abrin a A-subunit (ABa-A)-ricin B-subunit (RTB) with reduced or no toxicity.

Another particularly preferred module (a) or [module (a)+module (b)+module (c)] protein or peptide of the conjugate of the present invention comprises or consists of a hybrid toxin with reduced or no toxicity, wherein the hybrid toxin comprises a mutated A1-subunit of a first AB₅ toxin and a B-subunit of a second and different AB₅ toxin, wherein the first AB₅ toxin and the second and different AB₅ toxin are each selected from the group consisting of a cholera toxin, a Shiga toxin, a heat-labile enterotoxin, an E. coli heat-labile enterotoxin, and a pertussis toxin. Preferably, the hybrid AB₅ toxin with reduced or no toxicity comprises: a mutated A1-subunit of a cholera toxin and a B-subunit of a Shiga toxin, a heat-labile enterotoxin, an E. coli heat-labile enterotoxin, or a pertussis toxin; a mutated A1-subunit of a Shiga toxin and a B-subunit of a cholera toxin, a different Shiga toxin, a heat-labile enterotoxin, an E. coli heat-labile enterotoxin, or a pertussis toxin; a mutated A1-subunit of a heat-labile enterotoxin and a B-subunit of a cholera toxin, a Shiga toxin, an E. coli heat-labile enterotoxin, or a pertussis toxin; a mutated A1-subunit of an E. coli heat-labile enterotoxin and a B-subunit of a cholera toxin, a Shiga toxin, a heat-labile enterotoxin, or a pertussis toxin; or a mutated A1-subunit of a pertussis toxin and a B-subunit of a cholera toxin, a Shiga toxin, a heat-labile enterotoxin, or an E. coli heat-labile enterotoxin. Exemplary but not limiting embodiments of a hybrid AB₅ toxin include an A1(LT1)-A2(CT)-B₅(CT) toxin with reduced or no toxicity and an A1(Stx2a)-A2(Stx1a)-B₅(Stx1a) toxin with reduced or no toxicity.

In another preferred embodiment, the cell surface ligand of use as module (a) in a conjugate of the present invention is a molecule (e.g. natural ligand, short receptor binding peptide) that binds to a protein or peptide selected from the group consisting a TGN38/42, a CI-MPR (cation-independent mannose-6-phosphate receptor), a CD-MPR (cation-dependent mannose-6-phosphate receptor), a Sortilin protein or peptide, a polymeric IgA receptor, a Wnt protein or peptide, a Wnt1 protein or peptide, an apolipoprotein A-V protein or peptide, and an amyloid precursor protein or peptide.

Preferably, the cell surface ligand is a Wnt protein or peptide, a Wnt1 protein or peptide comprising or consisting of SEQ ID NO: 150 (human Wnt1; http://www.uniprot.org/uniprot/P04628), an apolipoprotein A-V protein or peptide comprising or consisting of an amino acid sequence of SEQ ID NO: 151 (http://www.uniprot.org/uniprot/Q6Q788), or an amyloid precursor protein or peptide comprising or consisting of SEQ ID NO: 152 (human APP; http://www.uniprot.org/uniprot/P05067) or an APP related protein or peptide.

A growth factor, lipoprotein, transferrin, surface binding lectin, galectin, c-type lectin, toxin, Wnt related protein or peptide, amyloid precursor protein, or apolipoprotein A-V variant differs from the wild-type growth factor, lipoprotein, transferrin, surface binding lectin, galectin, c-type lectin, toxin Wnt related protein or peptide, amyloid precursor protein, or apolipoprotein A-V protein or peptide from which it is derived by up to 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 200, 250, 300, 350, 400, 450, 500, 550 or 600 amino acid changes in the amino acid sequence (i.e. substitutions, insertions, deletions, N-terminal truncations and/or C-terminal truncations). Such a variant can alternatively or additionally be characterised by a certain degree of sequence identity to the wild-type protein from which it is derived. Thus, a growth factor, lipoprotein, transferrin, surface binding lectin, galectin, c-type lectin, toxin, Wnt related protein or peptide, amyloid precursor protein, or apolipoprotein A-V variant has a sequence identity of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% to the respective reference (wild-type) growth factor, lipoprotein, transferrin, surface binding lectin, galectin, c-type lectin, toxin, Wnt related protein or peptide, amyloid precursor protein, or apolipoprotein A-V.

A fragment (or deletion variant) of the growth factor, lipoprotein, transferrin, surface binding lectin, galectin, c-type lectin, toxin, Wnt related protein or peptide, amyloid precursor protein, or apolipoprotein A-V protein or peptide has preferably a deletion of up to 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 170, 200, 250, 300, 350, 400, 450, 500, 550 or 600 amino acids at its N-terminus and/or at its C-terminus and/or internally.

Additionally, a growth factor, lipoprotein, transferrin, surface binding lectin, galectin, c-type lectin, toxin, Wnt related protein or peptide, amyloid precursor protein, or apolipoprotein A-V protein or peptide variant or fragment is only regarded as a growth factor, lipoprotein, transferrin, surface binding lectin, galectin, c-type lectin, toxin, Wnt related protein or peptide, amyloid precursor protein, or apolipoprotein A-V protein or peptide variant or fragment within the context of the present invention, if it exhibits a relevant biological activity to a degree of at least 3 to 50%, preferably at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50% of the activity of the wild-type growth factor, lipoprotein, transferrin, surface binding lectin, galectin, c-type lectin or toxin protein. In a preferred embodiment, the growth factor, lipoprotein, transferrin, surface binding lectin, galectin, c-type lectin, toxin, Wnt related protein or peptide, amyloid precursor protein, or apolipoprotein A-V protein or peptide variant or fragment for use in a conjugate of the present invention, exhibits its relevant biological activity to a degree of at least 4 to 50%, at least 5 to 50%, at least 10 to 50%, at least 20 to 50%, at least 30 to 50%, at least 40 to 50%, or at least 45 to 50% of the activity of the wild-type growth factor, lipoprotein, transferrin, surface binding lectin, galectin, c-type lectin, toxin, Wnt related protein or peptide, amyloid precursor protein, or apolipoprotein A-V protein or peptide. The relevant “biological activity” in this context is the “activity to mediate cell targeting and to facilitate cellular uptake”, i.e. the ability of the variant or fragment to contact a cell and to enter the cell. One of ordinary skill in the art can readily assess whether a growth factor, lipoprotein, transferrin, surface binding lectin, galectin, c-type lectin, toxin, Wnt related protein or peptide, amyloid precursor protein, or apolipoprotein A-V protein or peptide variant or fragment has the ability to mediate cell targeting and to facilitate cellular uptake, i.e. at least 3 to 50%, at least 4 to 50%, at least 5 to 50%, at least 10 to 50%, at least 20 to 50%, at least 30 to 50%, at least 40 to 50%, or at least 45 to 50%, preferably at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50% of the activity of the wild-type growth factor, lipoprotein, transferrin, surface binding lectin, galectin, c-type lectin, toxin, Wnt related protein or peptide, amyloid precursor protein, on apolipoprotein A-V protein or peptide. Suitable assays, e.g. in vitro tracing of fluorescently labelled variants or fragments, for determining the “activity to mediate cell targeting and to facilitate cellular uptake” of a growth factor, lipoprotein, transferrin, surface binding lectin, galectin, c-type lectin, toxin, Wnt related protein or peptide, amyloid precursor protein, or apolipoprotein A-V protein or peptide variant or fragment compared to the binding activity of the respective wild-type protein are known to the person of ordinary skill in the art. Examples of suitable wild-type activity standards/in vitro tracing assays of use with the present invention are well described [for example, 14, 16 and 31-34), incorporated herein in their entirety and the like].

In another embodiment of the present invention, module (a), or the indirect targeting adapter molecule to which module (a) binds, comprises an antibody. Preferably, the antibody is selected from the group consisting of an anti-TGN38/46, an anti-transferrin receptor, and an anti-growth factor receptor, an anti-CI-MPR (cation-independent mannose-6-phosphate receptor), an anti-CD-MPR (cation-dependent mannose-6-phosphate receptor), an anti-Sortilin, an anti-polymeric IgA receptor, an anti-Wnt, an anti-Wnt1, an anti-apolipoprotein A-V, an anti-amyloid precursor, and an anti-pro-neurotrophin.

In another embodiment of the present invention, module (a), or the indirect targeting adapter molecule to which module (a) binds, comprises a sugar. Preferably, the sugar is selected from the group consisting of glucose, mannose, galactose, N-acetylglucosamine, N-acetylgalactosamine, fucose, N-acetylneuraminic acid and xylose.

In another embodiment of the present invention, module (a), or the indirect targeting adapter molecule to which module (a) binds, comprises a lipid. Preferably, the lipid is selected from the group consisting of a phospholipid, a glycolipid, a sphingolipid, and a sterol lipid.

In another embodiment of the present invention, module (a), or the indirect targeting adapter molecule to which module (a) binds, comprises a nanoparticle. Preferably, the nanoparticle is selected from the group consisting of a metal, a silicate, and a polymer. More preferably, the nanoparticle is a polymer selected from the group consisting of a poly(urethane), a poly(methyl methacrylate), a poly(vinyl alcohol), a poly(ethylene), a poly(vinyl pyrrolidone), a polylactide (PLA), a polyglycolide (PGA), a poly(lactide-co-glycolide) (PLGA), a polyanhydride and a polyorthoester.

In another embodiment of the present invention, module (a), or the indirect targeting adapter molecule to which module (a) binds, comprises a viral peptide that causes and/or results in specific cell targeting and cellular uptake. Preferably, said viral peptide is from a polyomavirus. More preferably, said viral peptide is from SV40, murine polyomavirus, BK virus, JC virus, KI virus, WU virus, and Merkel Cell polyomavirus. In the case of SV40, it has been shown to bind its cell surface receptor sialic acid on GM1 and its co-receptor MHC I, and is then transported to caveolae and from there into caveosomes; further transport brings SV40 into the smooth ER [35]. A second pathway has also been described in which SV40 avoids caveolae but exploits caveosomes to transport it from the caveosome to the ER [36]. Similar intracellular transport pathways have been described for the mouse polyomavirus (mPyV) and for other polyomaviruses [37]. Thus, a viral peptide, fragment or variant from SV40, murine polyomavirus, BK virus, JC virus, KI virus, WU virus, or Merkel Cell polyomavirus may be used as a module (a) or bound by a module (a) in the conjugates of the present invention.

The conjugate of the present invention comprises at least one module that facilitates the transport to the endoplasmic reticulum (ER), designated as module (b), and is preferably of human origin. Basically any molecule or structure that facilitates transport to the ER is suitable as module (b). Preferably, the module (b) of the conjugate of the present invention is an oligopeptide, preferably of human origin, which facilitates transport to the ER. In a conjugate of the present invention, module (b) can provide retrograde transport functionality either directly by comprising an oligopeptide that facilitates transport to the ER, or indirectly by binding to an endogenous protein, peptide or oligopeptide that facilitates transport to the ER (defined herein as an “endogenous ER transport protein, peptide or oligopeptide”).

The term “oligopeptide” in the context of the present invention means an amino acid sequence that comprises or consists of between 2 and 9 amino acid residues. Preferably, the oligopeptide of use with the conjugate of the present invention comprises between 2 and 9 amino acid residues in length. More preferably, the oligopeptide of use with the conjugate of the present invention comprises between 4 and 9 amino acid residues in length. More preferably, the oligopeptide of use with the conjugate of the present invention is 2, 3, 4, 5, 6, 7, 8 or 9 amino acid residues in length.

It is particularly preferred that the module (b), or the endogenous ER transport protein, peptide or oligopeptide to which module (b) binds, of the conjugate of the present invention comprises an oligopeptide comprising one or more of the amino acid sequence X₁X₂X₃X₄ (SEQ ID NO: 5), wherein X₁ is E, H, K, N, P, Q, R or S, preferably K or R; X₂ is D, E, A, T, V, G, S or N, preferably D or E; X₃ is E or D, preferably E; X₄ is L or F, preferably L, and wherein optionally the N-terminus and/or C-terminus comprises 1 to 3 additional amino acid residues.

More preferably, the module (b), or the endogenous ER transport protein, peptide or oligopeptide to which module (b) binds, of the conjugate of the present invention comprises an oligopeptide comprising one or more EDEL (SEQ ID NO: 6); HDEL (SEQ ID NO: 7); HEEL (SEQ ID NO: 8); KAEL (SEQ ID NO: 9); KDEF (SEQ ID NO: 10); KEDL (SEQ ID NO: 11); KEEL (SEQ ID NO: 12); KTEL (SEQ ID NO: 13); KVEL (SEQ ID NO: 14); NEDL (SEQ ID NO: 15); PDEL (SEQ ID NO: 16); PGEL (SEQ ID NO: 17); QEDL (SEQ ID NO: 18); QSEL (SEQ ID NO: 19); REDL (SEQ ID NO: 20); RNEL (SEQ ID NO: 21); RTDL (SEQ ID NO: 22); RTEL (SEQ ID NO: 23); ERSTEL (SEQ ID NO: 24); KDEL (SEQ ID NO: 25); AKDEL (SEQ ID NO: 26), PTEL (SEQ ID NO: 27); STEL (SEQ ID NO: 28); REDLK (SEQ ID NO: 29); or RDEL (SEQ ID NO: 30) motifs or variants thereof [38, 39].

The EDEL (SEQ ID NO: 6); HDEL (SEQ ID NO: 7); HEEL (SEQ ID NO: 8); KAEL (SEQ ID NO: 9); KDEF (SEQ ID NO: 10); KEDL (SEQ ID NO: 11); KEEL (SEQ ID NO: 12); KTEL (SEQ ID NO: 13); KVEL (SEQ ID NO: 14); NEDL (SEQ ID NO: 15); PDEL (SEQ ID NO: 16); PGEL (SEQ ID NO: 17); QEDL (SEQ ID NO: 18); QSEL (SEQ ID NO: 19); REDL (SEQ ID NO: 20); RNEL (SEQ ID NO: 21); RTDL (SEQ ID NO: 22); RTEL (SEQ ID NO: 23); ERSTEL (SEQ ID NO: 24); KDEL (SEQ ID NO: 25); AKDEL (SEQ ID NO: 26), PTEL (SEQ ID NO: 27); STEL (SEQ ID NO: 28); REDLK (SEQ ID NO: 29); or RDEL (SEQ ID NO: 30) motif variant differs from the respective wild-type motif from which it is derived by up to 1, 2, or 3 amino acid changes in the motif sequence (i.e. substitutions, insertions, deletions, N-terminal truncations and/or C-terminal truncations), preferably, conservative substitutions.

Additionally, said motif variant is only regarded as a motif variant within the context of the present invention, if it exhibits the relevant biological activity to a degree of at least 30%, preferably at least 50%, of the activity of the respective wild-type motif. The relevant “biological activity” in this context is the “activity to facilitate the transport to the endoplasmic reticulum (ER)”, i.e. the ability of the variant to target the conjugate to the endoplasmic recticulum (ER). The skilled person can readily assess whether an EDEL (SEQ ID NO: 6); HDEL (SEQ ID NO: 7); HEEL (SEQ ID NO: 8); KAEL (SEQ ID NO: 9); KDEF (SEQ ID NO: 10); KEDL (SEQ ID NO: 11); KEEL (SEQ ID NO: 12); KTEL (SEQ ID NO: 13); KVEL (SEQ ID NO: 14); NEDL (SEQ ID NO: 15); PDEL (SEQ ID NO: 16); PGEL (SEQ ID NO: 17); QEDL (SEQ ID NO: 18); QSEL (SEQ ID NO: 19); REDL (SEQ ID NO: 20); RNEL (SEQ ID NO: 21); RTDL (SEQ ID NO: 22); RTEL (SEQ ID NO: 23); ERSTEL (SEQ ID NO: 24); KDEL (SEQ ID NO: 25); AKDEL (SEQ ID NO: 26), PTEL (SEQ ID NO: 27); STEL (SEQ ID NO: 28); REDLK (SEQ ID NO: 29); or RDEL (SEQ ID NO: 30) motif variant has the ability to facilitate the transport to the ER, i.e. at least 30%, preferably at least 50%, of the activity of the respective wild-type motif. Suitable assays, e.g. in vitro tracing of fluorescently labelled variants, for determining the “activity to facilitate the transport to the endoplasmic reticulum (ER)” of an EDEL (SEQ ID NO: 6); HDEL (SEQ ID NO: 7); HEEL (SEQ ID NO: 8); KAEL (SEQ ID NO: 9); KDEF (SEQ ID NO: 10); KEDL (SEQ ID NO: 11); KEEL (SEQ ID NO: 12); KTEL (SEQ ID NO: 13); KVEL (SEQ ID NO: 14); NEDL (SEQ ID NO: 15); PDEL (SEQ ID NO: 16); PGEL (SEQ ID NO: 17); QEDL (SEQ ID NO: 18); QSEL (SEQ ID NO: 19); REDL (SEQ ID NO: 20); RNEL (SEQ ID NO: 21); RTDL (SEQ ID NO: 22); RTEL (SEQ ID NO: 23); ERSTEL (SEQ ID NO: 24); KDEL (SEQ ID NO: 25); AKDEL (SEQ ID NO: 26), PTEL (SEQ ID NO: 27); STEL (SEQ ID NO: 28); REDLK (SEQ ID NO: 29); or RDEL (SEQ ID NO: 30) variant compared to the binding activity of the respective wild-type motif are known to the person skilled in the art (see for example, [31]).

In another embodiment, module (b), or preferably the endogenous ER transport protein, peptide or oligopeptide to which module (b) binds, of the conjugate of the present invention is a Sortilin, SorLA, or S or CS protein, peptide or oligopeptide, or a fragment or variant thereof [40].

In another embodiment, module (b), or the endogenous ER transport protein, peptide or oligopeptide to which module (b) binds, of the conjugate of the present invention comprises a viral peptide that facilitates the transport to the ER. Preferably, said viral peptide is from a polyomavirus. More preferably, said viral peptide is from SV40, murine polyomavirus, BK virus, JC virus, KI virus, WU virus, and Merkel Cell polyomavirus. As described above, SV40 has been shown to bind its cell surface receptor sialic acid on GM1 and its co-receptor MHC I, and be transported to caveolae, then into caveosomes, and ultimately into the smooth ER [35]. SV40 has also been shown to avoid caveolae but exploit caveosomes to transport it from the caveosome to the ER [36]. Similar intracellular transport pathways have been described for the mouse polyomavirus (mPyV) and for other polyomaviruses [37]. Thus, a viral peptide, fragment or variant from SV40, murine polyomavirus, BK virus, JC virus, KI virus, WU virus, or Merkel Cell polyomavirus may be used as a module (b) or bound by module (b) in the conjugates of the present invention.

The conjugate of the present invention comprises or consists of at least one module that facilitates translocation from the endoplasmic reticulum (ER) to the cytosol (i.e., ERAD targeting), designated as module (c), and is preferably of mouse or human origin. Alternatively, module (c) can provide this ER to the cytosol translocation functionality indirectly by binding to an endogenous molecule that is capable of or is undergoing ERAD in the target cell. Examples of endogenous cellular molecule that may be bound by a module (c) of a conjugate of the present invention include but are not limited to COX2, Sgk1, null Hong Kong (NHK) variant of α1-antitrypsin (α1-AT), ASGPR H2a (a subunit of the asialoglycoprotein receptor), BACE457 [a pancreatic isoform of β-secretase (BACE)], CD3δ, TCRα, ΔF508 of CFTR (cystic fibrosis conductance regulator), HMG-CoA reductase (3-hydroxy-3-methyl-glutaryl-CoA reductase), IgK LC NS (a transport-incompetent immuno-globulin light chain), KAI1 (also known as CD82), MHC (major histocompatibility complex) class I molecules, Pael-R (Pael receptor), transthyretin (TTR [41], and the like (see for example, [42]).

In a preferred embodiment, module (c) binds to a cellular molecule that has a naturally short half-life due to rapid ERAD mediated degradation. Preferably, module (c) binds to an endogenous COX2 or Sgk1 protein or peptide.

Preferably, module (c) of the conjugate of the present invention comprises or consists of a protein or peptide selected from the group consisting of Cyclooxygenase-2 (COX2), Immunoglobulin M heavy chain [IgM(μ)], Igh6 [the rat homolog to IgM (0], Serum/glucocorticoid regulated kinase 1 (Sgk1), MATα2, Deg1, Mating pheromone alpha-factor 1 protein (MFα1; also referred to as yeast prepro-alpha factor), yeast carboxypeptidase (CPY), a toxin protein or peptide having reduced or no toxicity, an A/B type toxin protein or peptide having reduced or no toxicity, an A/B₅ type toxin protein or peptide having reduced or no toxicity, a toxin subunit having reduced or no toxicity, an A/B type toxin subunit having reduced or no toxicity, an A/B₅ type toxin subunit having reduced or no toxicity, a mutated toxin A-subunit having reduced or no toxicity, a non-toxic toxin A1-subunit, a mutated toxin A1-subunit having reduced or no toxicity, a toxin B-subunit, an α1-AT peptide, an ASGPR H2a peptide, a BACE457 peptide, a CD3δ peptide, a TCRα peptide, a ΔF508 of CFTR peptide, an HMG-CoA reductase peptide, an IgK LCNS peptide, a KAI1 (CD82) peptide, an MHC class I peptide, a Pael-R peptide, a transthyretin (TTR) peptide, a viral peptide, an acetylcholine esterase (AChE) peptide, a peptide fragment thereof, and a variant thereof.

In another embodiment, module (c) of the conjugate of the present invention is preferably selected from the group of C-terminal destabilizing oligopeptides consisting of CL1 (SEQ ID NO: 31), CL2 (SEQ ID NO: 32), CL6 (SEQ ID NO: 33), CL9 (SEQ ID NO: 34), CL10 (SEQ ID NO: 35), CL11 (SEQ ID NO: 36), CL12 (SEQ ID NO: 37), CL15 (SEQ ID NO: 38), CL16 (SEQ ID NO: 39), SL17 (SEQ ID NO: 40), a fragment thereof, and a variant thereof. Preferably, CL1 has the amino acid sequence ACKNWFSSLSHFVIHL (SEQ ID NO: 31); CL2 has the amino acid sequence SLISLPLPTRVKFSSLLLIRIMKIITMTFPKKLRS (SEQ ID NO: 32); CL6 has the amino acid sequence FYYPIWFARVLLVHYQ (SEQ ID NO: 33); CL9 has the amino acid sequence SNPFSSLFGASLLIDSVSLKSNWDTSSSSCLISFFSSVMFSSTTRS (SEQ ID NO: 34); CL10 has the amino acid sequence CRQRFSCHLTASYPQSTVTPFLAFLRRDFFFLR HNSSAD (SEQ ID NO: 35); CL11 has the amino acid sequence GAPHVVLFDFELRITNPLSHI QSVSLQITLIFCSLPSLILSKFLQV (SEQ ID NO: 36); CL12 has the amino acid sequence NTPLFSKSFSTTCGVAKKTLLLAQISSLFFLLLSSNIAV (SEQ ID NO: 37); CL15 has the amino acid sequence PTVKNSPKIFCLSSSPYLAFNLEYLSLRIFSTLSKCSNTLLTSLS (SEQ ID NO: 38); CL16 has the amino acid sequence SNQLKRLWLWLLEVRSFDRTLRRPWIHLPS (SEQ ID NO: 39); and SL17 has the amino acid sequence SISFVIRSHASIRMGASNDFFHKL YFTKCLTSVILSKFLIHLLLRSTPRV (SEQ ID NO: 40).

More preferably, the module (c) of the conjugate of the present invention comprises, essentially consists of or consists of
(a) a peptide of a protein selected from the group consisting of (COX2), IgM(μ), Sgk1, MATα2, MFα1, Igh6, Deg1, CPY, a toxin protein or peptide having reduced or no toxicity, an A/B type toxin protein or peptide having reduced or no toxicity, an A/B₅ type toxin protein or peptide having reduced or no toxicity, a toxin subunit having reduced or no toxicity, an A/B type toxin subunit having reduced or no toxicity, an A/B₅ type toxin subunit having reduced or no toxicity, a mutated toxin A-subunit having reduced or no toxicity, a non-toxic toxin A1-subunit, a mutated toxin A1-subunit having reduced or no toxicity, a toxin B-subunit, a mutated ricin toxin A-subunit (RTA) having reduced or no toxicity, a mutated ricin toxin A1-subunit (RTA1) having reduced or no toxicity, a ricin toxin B-subunit (RTB), a mutated cholera toxin A-subunit (CTA) having reduced or no toxicity, a mutated cholera toxin A1-subunit (CTA1) having reduced or no toxicity, a cholera toxin B-subunit (CTB), a mutated Shiga toxin (ST) A-subunit (STA) having reduced or no toxicity, a mutated Stx1a Shiga toxin A-subunit having reduced or no toxicity, a mutated Stx1b (VT1b) Shiga toxin A-subunit having reduced or no toxicity, a mutated Stx1c (VT1c) Shiga toxin A-subunit having reduced or no toxicity, a mutated Stx1d (VT1d) Shiga toxin A-subunit having reduced or no toxicity, a mutated Stx2a (VT2a) Shiga toxin A-subunit having reduced or no toxicity, a mutated Stx2b (VT2b) Shiga toxin A-subunit having reduced or no toxicity, a mutated Stx2c (VT2c) Shiga toxin A-subunit having reduced or no toxicity, a mutated Stx2d (VT2d) Shiga toxin A-subunit having reduced or no toxicity, a mutated Stx2e (VT2e) Shiga toxin A-subunit having reduced or no toxicity, a mutated Stx2f (VT2f) Shiga toxin A-subunit having reduced or no toxicity, a mutated Stx2g (VT2g) Shiga toxin A-subunit having reduced or no toxicity, a mutated Shiga toxin A1-subunit (STA1) having reduced or no toxicity, a mutated Stx1a Shiga toxin A1-subunit having reduced or no toxicity, a mutated Stx1b (VT1b) Shiga toxin A1-subunit having reduced or no toxicity, a mutated Stx1c (VT1c) Shiga toxin A1-subunit having reduced or no toxicity, a mutated Stx1d (VT1d) Shiga toxin A1-subunit having reduced or no toxicity, a mutated Stx2a (VT2a) Shiga toxin A1-subunit having reduced or no toxicity, a mutated Stx2b (VT2b) Shiga toxin A1-subunit having reduced or no toxicity, a mutated Stx2c (VT2c) Shiga toxin A1-subunit having reduced or no toxicity, a mutated Stx2d (VT2d) Shiga toxin A1-subunit having reduced or no toxicity, a mutated Stx2e (VT2e) Shiga toxin A1-subunit having reduced or no toxicity, a mutated Stx2f (VT2f) Shiga toxin A1-subunit having reduced or no toxicity, and a mutated Stx2g (VT2g) Shiga toxin A1-subunit having reduced or no toxicity, a Shiga toxin A1-subunit peptide, an Stx1a Shiga toxin A1-subunit peptide, an Stx1b (VT1b) Shiga toxin A1-subunit peptide, an Stx1c (VT1c) Shiga toxin A1-subunit peptide, an Stx1d (VT1d) Shiga toxin A1-subunit peptide, an Stx2a (VT2a) Shiga toxin A1-subunit peptide, an Stx2b (VT2b) Shiga toxin A1-subunit peptide, an Stx2c (VT2c) Shiga toxin A1-subunit peptide, an Stx2d (VT2d) Shiga toxin A1-subunit peptide, an Stx2e (VT2e) Shiga toxin A1-subunit peptide, an Stx2f (VT2f) Shiga toxin A1-subunit peptide, an Stx2g (VT2g) Shiga toxin A1-subunit peptide, a Shiga toxin B-subunit (STB), an Stx1a Shiga toxin B-subunit, an Stx1b (VT1b) Shiga toxin B-subunit, an Stx1c (VT1c) Shiga toxin B-subunit, an Stx1d (VT1d) Shiga toxin B-subunit, an Stx2a (VT2a) Shiga toxin B-subunit, an Stx2b (VT2b) Shiga toxin B-subunit, an Stx2c (VT2c) Shiga toxin B-subunit, an Stx2d (VT2d) Shiga toxin B-subunit, an Stx2e (VT2e) Shiga toxin B-subunit, an Stx2g (VT2g) Shiga toxin B-subunit, a Shiga toxin B-subunit peptide, an Stx1a Shiga toxin B-subunit peptide, an Stx1b (VT1b) Shiga toxin B-subunit peptide, an Stx1c (VT1c) Shiga toxin B-subunit peptide, an Stx1d (VT1d) Shiga toxin B-subunit peptide, an Stx2a (VT2a) Shiga toxin B-subunit peptide, an Stx2b (VT2b) Shiga toxin B-subunit peptide, an Stx2c (VT2c) Shiga toxin B-subunit peptide, an Stx2d (VT2d) Shiga toxin B-subunit peptide, an Stx2e (VT2e) Shiga toxin B-subunit peptide, an Stx2f (VT2f) Shiga toxin B-subunit peptide, an Stx2g (VT2g) Shiga toxin B-subunit peptide, a mutated Escherichia coli heat labile enterotoxin (LT) A-subunit (LT-A) having reduced or no toxicity, a mutated LT-IIa A-subunit having reduced or no toxicity, a mutated LT-IIa A-subunit peptide having reduced or no toxicity, a mutated LT-IIb A-subunit having reduced or no toxicity, an LT B-subunit (LT-B), an LT-IIa B-subunit, an LT-IIb B-subunit, a mutated Abrin-a A-subunit having reduced or no toxicity, a mutated Abrin-b A-subunit having reduced or no toxicity, a mutated Abrin-c A-subunit having reduced or no toxicity, a mutated Abrin-d A-subunit having reduced or no toxicity, a mutated Pertussis A-subunit having reduced or no toxicity, a Pertussis B-subunit, a mutated Modeccin A-subunit having reduced or no toxicity, a Modeccin B-subunit, a mutated Volkensin A-subunit having reduced or no toxicity, a Volkensin B-subunit, a mutated Viscumin A-subunit having reduced or no toxicity, a Viscumin B-subunit, a mutated Pseudomonas Exotoxin A protein or peptide having reduced or no toxicity, a Pseudomonas Exotoxin A Domain II, a mutated Escherichia coli subtilase cytotoxin A-subunit having reduced or no toxicity, an Escherichia coli subtilase cytotoxin B-subunit, a mutated Cinnamomin I toxin A-subunit having reduced or no toxicity, a mutated Cinnamomin II toxin A-subunit having reduced or no toxicity, a mutated Cinnamomin III toxin A-subunit having reduced or no toxicity, a mutated Sambucus ribosome-inactivating protein A-subunit having reduced or no toxicity, a mutated ribosome-inactivating protein SNAI′ A-subunit having reduced or no toxicity, a mutated Ebulin 1 ribosome-inactivating protein (ebu1) A-subunit having reduced or no toxicity, a mutated type 2 ribosome-inactivating protein SNAIf A-subunit having reduced or no toxicity, a mutated lectin [Q41358 (Q41358—SAMNI)] A-subunit having reduced or no toxicity, a mutated ribosome-inactivating protein (AV1) A-subunit having reduced or no toxicity, a mutated type 2 ribosome-inactivating protein Nigrin 1 A-subunit having reduced or no toxicity, a mutated type 2 ribosome-inactivating protein Nigrin b A-subunit having reduced or no toxicity, a mutated Bodinierin toxin A-subunit having reduced or no toxicity, a mutated Porrectin toxin A-subunit having reduced or no toxicity, a mutated cinphorin toxin A-subunit with reduced or no toxicity, α1-AT peptide, ASGPR H2a peptide, BACE457 peptide, CD3δ peptide, TCRα peptide, ΔF508 of CFTR peptide, HMG-CoA reductase peptide, IgK LCNS peptide, KAI1 (CD82) peptide, MHC class I peptide, Pael-R peptide, transthyretin (TTR) peptide, viral peptide, SV40 viral peptide, murine polyomavirus peptide, BK viral peptide, JC viral peptide, KI viral peptide, WU viral peptide, Merkel Cell polyomavirus peptide, an AChE peptide, fragments thereof, and variants thereof, or
(b) a peptide comprising, essentially consisting of or consisting of the amino acid sequence

CL1 (SEQ ID NO: 31), CL2 (SEQ ID NO: 32), CL6 (SEQ ID NO: 33), CL9 (SEQ ID NO: 34), CL10 (SEQ ID NO: 35), CL11 (SEQ ID NO₃₆), CL12 (SEQ ID NO: 37), CL15 (SEQ ID NO: 38), CL16 (SEQ ID NO: 39), SL17 (SEQ ID NO: 40), or a fragment or variant thereof.

A COX2, IgM(μ), Sgk1, MATα2, MFα1, Igh6, Deg1, CPY, toxin protein or peptide having reduced or no toxicity, A/B type toxin protein or peptide having reduced or no toxicity, A/B₅ type toxin protein or peptide having reduced or no toxicity, toxin subunit having reduced or no toxicity, toxin domain having reduced or no toxicity, A/B type toxin subunit having reduced or no toxicity, A/B₅ type toxin subunit having reduced or no toxicity, mutated toxin A-subunit having reduced or no toxicity, non-toxic toxin A1-subunit, mutated toxin A1-subunit having reduced or no toxicity, toxin B-subunit, α1-AT peptide, ASGPR H2a peptide, BACE457 peptide, CD3δ peptide, TCRα peptide, ΔF508 of CFTR peptide, HMG-CoA reductase peptide, IgK LCNS peptide, KAI1 (CD82) peptide, MHC class I peptide, Pael-R peptide, transthyretin (TTR) peptide, viral peptide, SV40 viral peptide, murine polyomavirus peptide, BK viral peptide, JC viral peptide, KI viral peptide, WU viral peptide, Merkel Cell polyomavirus, or AChE peptide variant differs from the respective wild-type COX2, IgM(μ), Sgk1, MATα2, MFα1, Igh6, Deg1, CPY, toxin protein or peptide, A/B type toxin protein or peptide, A/B₅ type toxin protein or peptide, toxin subunit, toxin domain, A/B type toxin subunit, A/B₅ type toxin subunit, toxin A-subunit, toxin A1-subunit, toxin B-subunit, α1-AT peptide, ASGPR H2a peptide, BACE457 peptide, CD3δ peptide, TCRα peptide, ΔF508 of CFTR peptide, HMG-CoA reductase peptide, IgK LCNS peptide, KAI1 (CD82) peptide, MHC class I peptide, Pael-R peptide, transthyretin (TTR) peptide, viral peptide, SV40 viral peptide, murine polyomavirus peptide, BK viral peptide, JC viral peptide, KI viral peptide, WU viral peptide, Merkel Cell polyomavirus, or AChE peptide or protein, respectively, in that the variant comprises an amino acid sequence comprising up to 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 148, 150, 160, 170, 180, 190, 200, 220, 250, 270, 300, 331, 350, 368, 370, 371, 387, 400, 410, 415, 417, 420, 422, 424, 435, 440, 450, 470, 500, 504, 505, 510, 515, 520, 550, 560, 570, 579, 585 or 590 amino acid changes in the variant's amino acid sequence (i.e. substitutions, insertions, deletions, N-terminal truncations and/or C-terminal truncations) as compared to its corresponding wild-type protein's/peptide's amino acid sequence. Such a variant can alternatively or additionally be characterized by a certain degree of sequence identity to the wild-type protein from which it is derived. Thus, a COX2, IgM(μ), Sgk1, MATα2, MFα1, Igh6, Deg1, CPY, toxin protein or peptide, A/B type toxin protein or peptide, A/B₅ type toxin protein or peptide, toxin subunit, toxin domain, A/B type toxin subunit, A/B₅ type toxin subunit, toxin A-subunit, toxin A1-subunit, toxin B-subunit, α1-AT peptide, ASGPR H2a peptide, BACE457 peptide, CD3δ peptide, TCRα peptide, ΔF508 of CFTR peptide, HMG-CoA reductase peptide, IgK LCNS peptide, KAI1 (CD82) peptide, MHC class I peptide, Pael-R peptide, transthyretin (TTR) peptide, viral peptide, SV40 viral peptide, murine polyomavirus peptide, BK viral peptide, JC viral peptide, KI viral peptide, WU viral peptide, Merkel Cell polyomavirus or AChE peptide variant has a sequence identity of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% to the respective reference (wild-type) COX2, IgM(μ), Sgk1, MATα2, MFα1, Igh6, Deg1, CPY, toxin protein or peptide, A/B type toxin protein or peptide, A/B₅ type toxin protein or peptide, toxin subunit, toxin domain, A/B type toxin subunit, A/B₅ type toxin subunit, toxin A-subunit, toxin A1-subunit, toxin B-subunit, α1-AT peptide, ASGPR H2a peptide, BACE457 peptide, CD36 peptide, TCRα peptide, ΔF508 of CFTR peptide, HMG-CoA reductase peptide, IgK LCNS peptide, KAI1 (CD82) peptide, MHC class I peptide, Pael-R peptide, transthyretin (TTR) peptide, viral peptide, SV40 viral peptide, murine polyomavirus peptide, BK viral peptide, JC viral peptide, KI viral peptide, WU viral peptide, Merkel Cell polyomavirus, or AChE peptide amino acid sequence.

A peptide fragment (or deletion variant) of the COX2, IgM(μ), Sgk1, MATα2, MFα1, Igh6, Deg1, CPY, toxin protein or peptide, A/B type toxin protein or peptide, A/B₅ type toxin protein or peptide, toxin subunit, toxin domain, A/B type toxin subunit, A/B₅ type toxin subunit, toxin A-subunit, toxin A1-subunit, toxin B-subunit, ricin toxin A-subunit (RTA), ricin toxin A1-subunit (RTA1), ricin toxin B-subunit (RTB), cholera toxin A-subunit (CTA), cholera toxin A1-subunit (CTA1), cholera toxin B-subunit (CTB), Shiga toxin (ST) A-subunit (STA), Stx1a Shiga toxin A-subunit, Stx1b (VT1b) Shiga toxin A-subunit, Stx1c (VT1c) Shiga toxin A-subunit, Stx1d (VT1d) Shiga toxin A-subunit, Stx2a (VT2a) A-subunit, Stx2b (VT2b) Shiga toxin A-subunit, Stx2c (VT2c) Shiga toxin A-subunit, a Stx2d (VT2d) Shiga toxin A-subunit, Stx2e (VT2e) Shiga toxin A-subunit, Stx2f (VT2f) Shiga toxin A-subunit, Stx2g (VT2g) Shiga toxin A-subunit, Shiga toxin A1-subunit (STA1), Stx1a Shiga toxin A1-subunit, Stx1b (VT1b) Shiga toxin A 1-subunit, Stx1c (VT1c) Shiga toxin A 1-subunit, Stx1d (VT1d) Shiga toxin A 1-subunit, Stx2a (VT2a) Shiga toxin A1-subunit, Stx2b (VT2b) Shiga toxin A1-subunit, Stx2c (VT2c) Shiga toxin A1-subunit, a Stx2d (VT2d) Shiga toxin A1-subunit, Stx2e (VT2e) Shiga toxin A1-subunit, Stx2f (VT2f) Shiga toxin A1-subunit, Stx2g (VT2g) Shiga toxin A1-subunit, a Shiga toxin B-subunit (STB), an Stx1a Shiga toxin B-subunit, an Stx1b (VT1b) Shiga toxin B-subunit, an Stx1c (VT1c) Shiga toxin B-subunit, an Stx1d (VT1d) Shiga toxin B-subunit, an Stx2a (VT2a) Shiga toxin B-subunit, an Stx2b (VT2b) Shiga toxin B-subunit, an Stx2c (VT2c) Shiga toxin B-subunit, an Stx2d (VT2d) Shiga toxin B-subunit, an Stx2e (VT2e) Shiga toxin B-subunit, an Stx2f (VT2f) Shiga toxin B-subunit, an Stx2g (VT2g) Shiga toxin B-subunit, Escherichia coli heat labile enterotoxin (LT) A-subunit (LT-A), LT-IIa A-subunit, LT-IIa A-subunit peptide, LT-IIb A-subunit, LT B-subunit (LT-B), LT-IIa B-subunit, LT-IIb B-subunit, Abrin-a A-subunit, Abrin-b A-subunit, Abrin-c A-subunit, Abrin-d A-subunit, pertussis A-subunit, pertussis B-subunit, Modeccin A-subunit, Modeccin B-subunit, Volkensin A-subunit, Volkensin B-subunit, Viscumin A-subunit, Viscumin B-subunit, Pseudomonas Exotoxin A, Pseudomonas Exotoxin A Domain II, Escherichia coli subtilase cytotoxin A-subunit, Escherichia coli subtilase cytotoxin B-subunit, Cinnamomin I toxin A-subunit, Cinnamomin II toxin A-subunit, Cinnamomin III toxin A-subunit, Sambucus ribosome-inactivating protein A-subunit, ribosome-inactivating protein SNAI′ A-subunit, Ebulin 1 ribosome-inactivating protein (ebu1) A-subunit, type 2 ribosome-inactivating protein SNAIf A-subunit, lectin [Q41358 (Q41358 SAMNI)] A-subunit, ribosome-inactivating protein (AV1) A-subunit, type 2 ribosome-inactivating protein Nigrin 1 A-subunit, type 2 ribosome-inactivating protein Nigrin b A-subunit, Bodinierin toxin A-subunit, Porrectin toxin A-subunit, cinphorin toxin A-subunit toxin protein or peptide, α1-AT peptide, ASGPR H2a peptide, BACE457 peptide, CD3δ peptide, TCRα peptide, ΔF508 of CFTR peptide, HMG-CoA reductase peptide, IgK LCNS peptide, KAI1 (CD82) peptide, MHC class I peptide, Pael-R peptide, transthyretin (TTR) peptide, viral peptide, SV40 viral peptide, murine polyomavirus peptide, BK viral peptide, JC viral peptide, KI viral peptide, WU viral peptide, Merkel Cell polyomavirus, or AChE protein or peptide preferably has a deletion of up to 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 148, 150, 160, 170, 180, 190, 200, 220, 250, 270, 300, 331, 350, 368, 370, 371, 387, 400, 410, 415, 417, 420, 422, 424, 435, 440, 450, 470, 500, 504, 505, 510, 515, 520, 550, 560, 570, 579, 585 or 590 amino acids at its N-terminus and/or at its C-terminus and/or internally.

Additionally, a COX2, IgM(μ), Sgk1, MATα2, MFα1, Igh6, Deg1, CPY, toxin protein or peptide, A/B type toxin protein or peptide, A/B₅ type toxin protein or peptide, toxin subunit, toxin domain, A/B type toxin subunit, A/B₅ type toxin subunit, toxin A-subunit, toxin A1-subunit, toxin B-subunit, ricin toxin A-subunit (RTA), ricin toxin A1-subunit (RTA1), ricin toxin B-subunit (RTB), cholera toxin A-subunit (CTA), cholera toxin A1-subunit (CTA1), cholera toxin B-subunit (CTB), Shiga toxin (ST) A-subunit (STA), Stx1a Shiga toxin A-subunit, Stx1b (VT1b) Shiga toxin A-subunit, Stx1c (VT1c) Shiga toxin A-subunit, Stx1d (VT1d) Shiga toxin A-subunit, Stx2a (VT2a) A-subunit, Stx2b (VT2b) Shiga toxin A-subunit, Stx2c (VT2c) Shiga toxin A-subunit, a Stx2d (VT2d) Shiga toxin A-subunit, Stx2e (VT2e) Shiga toxin A-subunit, Stx2f (VT2f) Shiga toxin A-subunit, Stx2g (VT2g) Shiga toxin A-subunit, Shiga toxin A1-subunit (STA1), Stx1a Shiga toxin A1-subunit, Stx1b (VT1b) Shiga toxin A1-subunit, Stx1c (VT1c) Shiga toxin A1-subunit, Stx1d (VT1d) Shiga toxin A1-subunit, Stx2a (VT2a) Shiga toxin A1-subunit, Stx2b (VT2b) Shiga toxin A1-subunit, Stx2c (VT2c) Shiga toxin A1-subunit, a Stx2d (VT2d) Shiga toxin A1-subunit, Stx2e (VT2e) Shiga toxin A1-subunit, Stx2f (VT2f) Shiga toxin A1-subunit, Stx2g (VT2g) Shiga toxin A1-subunit, a Shiga toxin B-subunit (STB), an Stx1a Shiga toxin B-subunit, an Stx1b (VT1b) Shiga toxin B-subunit, an Stx1c (VT1c) Shiga toxin B-subunit, an Stx1d (VT1d) Shiga toxin B-subunit, an Stx2a (VT2a) Shiga toxin B-subunit, an Stx2b (VT2b) Shiga toxin B-subunit, an Stx2c (VT2c) Shiga toxin B-subunit, an Stx2d (VT2d) Shiga toxin B-subunit, an Stx2e (VT2e) Shiga toxin B-subunit, an Stx2f (VT2f) Shiga toxin B-subunit, an Stx2g (VT2g) Shiga toxin B-subunit, Escherichia coli heat labile enterotoxin (LT) A-subunit (LT-A), LT-IIa A-subunit, LT-IIa A-subunit peptide, LT-IIb A-subunit, LT B-subunit (LT-B), LT-IIa B-subunit, LT-IIb B-subunit, Abrin-a A-subunit, Abrin-b A-subunit, Abrin-c A-subunit, Abrin-d A-subunit, pertussis A-subunit, pertussis B-subunit, Modeccin A-subunit, Modeccin B-subunit, Volkensin A-subunit, Volkensin B-subunit, Viscumin A-subunit, Viscumin B-subunit, Pseudomonas Exotoxin A, Pseudomonas Exotoxin A Domain II, Escherichia coli subtilase cytotoxin A-subunit, Escherichia coli subtilase cytotoxin B-subunit, Cinnamomin I toxin A-subunit, Cinnamomin II toxin A-subunit, Cinnamomin III toxin A-subunit, Sambucus ribosome-inactivating protein A-subunit, ribosome-inactivating protein SNAI′ A-subunit, Ebulin 1 ribosome-inactivating protein (ebu1) A-subunit, type 2 ribosome-inactivating protein SNAIf A-subunit, lectin [Q41358 (Q41358—SAMNI)] A-subunit, ribosome-inactivating protein (AV1) A-subunit, type 2 ribosome-inactivating protein Nigrin 1 A-subunit, type 2 ribosome-inactivating protein Nigrin b A-subunit, Bodinierin toxin A-subunit, Porrectin toxin A-subunit, cinphorin toxin A-subunit toxin protein or peptide, α1-AT peptide, ASGPR H2a peptide, BACE457 peptide, CD3δ peptide, TCRα peptide, ΔF508 of CFTR peptide, HMG-CoA reductase peptide, IgK LCNS peptide, KAI1 (CD82) peptide, MHC class I peptide, Pael-R peptide, transthyretin (TTR) peptide, viral peptide, SV40 viral peptide, murine polyomavirus peptide, BK viral peptide, JC viral peptide, KI viral peptide, WU viral peptide, Merkel Cell polyomavirus, or AChE protein/peptide variant or protein/peptide fragment is only regarded as a COX2, IgM(μ), Sgk1, MATalpha2, MATα2, MFα1, Igh6, Deg1, CPY, toxin protein or peptide, A/B type toxin protein or peptide, A/B₅ type toxin protein or peptide, toxin subunit, toxin domain, A/B type toxin subunit, A/B₅ type toxin subunit, toxin A-subunit, toxin A1-subunit, toxin B-subunit, ricin toxin A-subunit (RTA), ricin toxin A1-subunit (RTA1), ricin toxin B-subunit (RTB), cholera toxin A-subunit (CTA), cholera toxin A1-subunit (CTA1), cholera toxin B-subunit (CTB), Shiga toxin (ST) A-subunit (STA), Stx1a Shiga toxin A-subunit, Stx1b (VT1b) Shiga toxin A-subunit, Stx1c (VT1c) Shiga toxin A-subunit, Stx1d (VT1d) Shiga toxin A-subunit, Stx2a (VT2a) A-subunit, Stx2b (VT2b) Shiga toxin A-subunit, Stx2c (VT2c) Shiga toxin A-subunit, a Stx2d (VT2d) Shiga toxin A-subunit, Stx2e (VT2e) Shiga toxin A-subunit, Stx2f (VT2f) Shiga toxin A-subunit, Stx2g (VT2g) Shiga toxin A-subunit, Shiga toxin A1-subunit (STA1), Stx1a Shiga toxin A1-subunit, Stx1b (VT1b) Shiga toxin A1-subunit, Stx1c (VT1c) Shiga toxin A1-subunit, Stx1d (VT1d) Shiga toxin A1-subunit, Stx2a (VT2a) Shiga toxin A1-subunit, Stx2b (VT2b) Shiga toxin A1-subunit, Stx2c (VT2c) Shiga toxin A1-subunit, a Stx2d (VT2d) Shiga toxin A1-subunit, Stx2e (VT2e) Shiga toxin A1-subunit, Stx2f (VT2f) Shiga toxin A1-subunit, Stx2g (VT2g) Shiga toxin A1-subunit, a Shiga toxin B-subunit (STB), an Stx1a Shiga toxin B-subunit, an Stx1b (VT1b) Shiga toxin B-subunit, an Stx1c (VT1c) Shiga toxin B-subunit, an Stx1d (VT1d) Shiga toxin B-subunit, an Stx2a (VT2a) Shiga toxin B-subunit, an Stx2b (VT2b) Shiga toxin B-subunit, an Stx2c (VT2c) Shiga toxin B-subunit, an Stx2d (VT2d) Shiga toxin B-subunit, an Stx2e (VT2e) Shiga toxin B-subunit, an Stx2f (VT2f) Shiga toxin B-subunit, an Stx2g (VT2g) Shiga toxin B-subunit, Escherichia coli heat labile enterotoxin (LT) A-subunit (LT-A), LT-IIa A-subunit, LT-IIa A-subunit peptide, LT-IIb A-subunit, LT B-subunit (LT-B), LT-IIa B-subunit, LT-IIb B-subunit, Abrin-a A-subunit, Abrin-b A-subunit, Abrin-c A-subunit, Abrin-d A-subunit, pertussis A-subunit, pertussis B-subunit, Modeccin A-subunit, Modeccin B-subunit, Volkensin A-subunit, Volkensin B-subunit, Viscumin A-subunit, Viscumin B-subunit, Pseudomonas Exotoxin A, Pseudomonas Exotoxin A Domain II, Escherichia coli subtilase cytotoxin A-subunit, Escherichia coli subtilase cytotoxin B-subunit, Cinnamomin I toxin A-subunit, Cinnamomin II toxin A-subunit, Cinnamomin III toxin A-subunit, Sambucus ribosome-inactivating protein A-subunit, ribosome-inactivating protein SNAI′ A-subunit, Ebulin 1 ribosome-inactivating protein (ebu1) A-subunit, type 2 ribosome-inactivating protein SNAIf A-subunit, lectin [Q41358 (Q41358—SAMNI)] A-subunit, ribosome-inactivating protein (AV1) A-subunit, type 2 ribosome-inactivating protein Nigrin 1 A-subunit, type 2 ribosome-inactivating protein Nigrin b A-subunit, Bodinierin toxin A-subunit, Porrectin toxin A-subunit, cinphorin toxin A-subunit toxin protein or peptide, α1-AT peptide, ASGPR H2a peptide, BACE457 peptide, CD3δ peptide, TCRα peptide, ΔF508 of CFTR peptide, HMG-CoA reductase peptide, IgK LCNS peptide, KAI1 (CD82) peptide, MHC class I peptide, Pael-R peptide, transthyretin (TTR) peptide, viral peptide, SV40 viral peptide, murine polyomavirus peptide, BK viral peptide, JC viral peptide, KI viral peptide, WU viral peptide, Merkel Cell polyomavirus, AChE protein/peptide variant or protein/peptide fragment within the context of the present invention, if it exhibits the relevant biological activity to a degree of at least 30%, preferably at least 50% of the activity of the corresponding wild-type COX2, IgM(μ), Sgk1, MATα2, MFα1, Igh6, Deg1, CPY, toxin protein or peptide, A/B type toxin protein or peptide, A/B₅ type toxin protein or peptide, toxin subunit, A/B type toxin subunit, A/B₅ type toxin subunit, toxin domain, toxin A-subunit, toxin A1-subunit, toxin B-subunit, ricin toxin A-subunit (RTA), ricin toxin A1-subunit (RTA1), ricin toxin B-subunit (RTB), cholera toxin A-subunit (CTA), cholera toxin A1-subunit (CTA1), cholera toxin B-subunit (CTB), Shiga toxin (ST) A-subunit (STA), Stx1a Shiga toxin A-subunit, Stx1b (VT1b) Shiga toxin A-subunit, Stx1c (VT1c) Shiga toxin A-subunit, Stx1d (VT1d) Shiga toxin A-subunit, Stx2a (VT2a) A-subunit, Stx2b (VT2b) Shiga toxin A-subunit, Stx2c (VT2c) Shiga toxin A-subunit, a Stx2d (VT2d) Shiga toxin A-subunit, Stx2e (VT2e) Shiga toxin A-subunit, Stx2f (VT2f) Shiga toxin A-subunit, Stx2g (VT2g) Shiga toxin A-subunit, Shiga toxin A1-subunit (STA1), Stx1a Shiga toxin A1-subunit, Stx1b (VT1b) Shiga toxin A1-subunit, Stx1c (VT1c) Shiga toxin A1-subunit, Stx1d (VT1d) Shiga toxin A1-subunit, Stx2a (VT2a) Shiga toxin A1-subunit, Stx2b (VT2b) Shiga toxin A1-subunit, Stx2c (VT2c) Shiga toxin A1-subunit, a Stx2d (VT2d) Shiga toxin A1-subunit, Stx2e (VT2e) Shiga toxin A1-subunit, Stx2f (VT2f) Shiga toxin A1-subunit, Stx2g (VT2g) Shiga toxin A1-subunit, a Shiga toxin B-subunit (STB), an Stx1a Shiga toxin B-subunit, an Stx1b (VT1b) Shiga toxin B-subunit, an Stx1c (VT1c) Shiga toxin B-subunit, an Stx1d (VT1d) Shiga toxin B-subunit, an Stx2a (VT2a) Shiga toxin B-subunit, an Stx2b (VT2b) Shiga toxin B-subunit, an Stx2c (VT2c) Shiga toxin B-subunit, an Stx2d (VT2d) Shiga toxin B-subunit, an Stx2e (VT2e) Shiga toxin B-subunit, an Stx2f (VT2f) Shiga toxin B-subunit, an Stx2g (VT2g) Shiga toxin B-subunit, Escherichia coli heat labile enterotoxin (LT) A-subunit (LT-A), LT-IIa A-subunit, LT-IIa A-subunit peptide, LT-IIb A-subunit, LT B-subunit (LT-B), LT-IIa B-subunit, LT-IIb B-subunit, Abrin-a A-subunit, Abrin-b A-subunit, Abrin-c A-subunit, Abrin-d A-subunit, pertussis A-subunit, pertussis B-subunit, Modeccin A-subunit, Modeccin B-subunit, Volkensin A-subunit, Volkensin B-subunit, Viscumin A-subunit, Viscumin B-subunit, Pseudomonas Exotoxin A, Pseudomonas Exotoxin A Domain II, Escherichia coli subtilase cytotoxin A-subunit, Escherichia coli subtilase cytotoxin B-subunit, Cinnamomin I toxin A-subunit, Cinnamomin II toxin A-subunit, Cinnamomin III toxin A-subunit, Sambucus ribosome-inactivating protein A-subunit, ribosome-inactivating protein SNAI′ A-subunit, Ebulin 1 ribosome-inactivating protein (ebu1) A-subunit, type 2 ribosome-inactivating protein SNAIf A-subunit, lectin [Q41358 (Q41358—SAMNI)] A-subunit, ribosome-inactivating protein (AV1) A-subunit, type 2 ribosome-inactivating protein Nigrin 1 A-subunit, type 2 ribosome-inactivating protein Nigrin b A-subunit, Bodinierin toxin A-subunit, Porrectin toxin A-subunit, cinphorin toxin A-subunit toxin protein or peptide, α1-AT peptide, ASGPR H2a peptide, BACE457 peptide, CD3δ peptide, TCRα peptide, ΔF508 of CFTR peptide, HMG-CoA reductase peptide, IgK LCNS peptide, KAI1 (CD82) peptide, MHC class I peptide, Pael-R peptide, transthyretin (TTR) peptide, viral peptide, SV40 viral peptide, murine polyomavirus peptide, BK viral peptide, JC viral peptide, KI viral peptide, WU viral peptide, Merkel Cell polyomavirus, AChE respectively. The relevant “biological activity” in this context is the “activity to mediate translocation from the endoplasmic reticulum (ER) to the cytosol”, i.e. the ability of the variant or fragment to translocate from the lumen of the ER in the cytosol of a cell.

One of ordinary skill in the art can readily assess whether a protein/peptide variant or protein/peptide fragment according to the present invention has the ability to translocate from the lumen of the ER in the cytosol, i.e. at least 30%, preferably at least 50% of the activity of its corresponding wild-type protein/peptide. Suitable assays, e.g. in vitro tracing of variants or fragments, for determining the “activity to mediate translocation from the endoplasmic reticulum (ER) to the cytosol” of a protein/peptide, protein/peptide variant or protein/peptide fragment according to the invention compared to the binding activity of the respective wild-type protein/peptide are known in the art (see for example, [17]).

A peptide fragment of the COX2 protein has preferably a deletion of up to 1, 2, 3, 4, 5, 6, 7, 8, 9, 10, 15, 20, 25, 30, 35, 40, 45, 50, 55, 60, 65, 70, 75, 80, 85, 90, 95, 100, 120, 150, 170, 200, 220, 250, 270, 300, 350, 370, 400, 420, 450, 470, 500, 504, 520, 550, 560, 570, 579, 585 or 590 amino acids at its N-terminus and/or at its C-terminus and/or internally, preferably at its N-terminus.

A peptide fragment of the IgM(μ) protein has preferably a deletion of up to 1, 2, 3, 4, 5, 6, 7, 8, 9, 10, 15, 20, 25, 30, 35, 40, 45, 50, 55, 60, 65, 70, 75, 80, 85, 90, 95, 100, 120, 150, 170, 200, 250, 270, 300, 320, 350, 360, 370, 380, 390, 400, 410, 420, 435 or 440 amino acids at its N-terminus and/or at its C-terminus and/or internally, preferably at its N-terminus

A peptide fragment of the Sgk1 protein has preferably a deletion of up to 1, 2, 3, 4, 5, 6, 7, 8, 9, 10, 15, 20, 25, 30, 35, 40, 45, 50, 55, 60, 65, 70, 75, 80, 85, 90, 95, 100, 120, 150, 170, 200, 220, 250, 270, 300, 320, 325, 331, 350, 360, 368, 371, 380, 387, 400, 410, 415, 417, 422, or 424 amino acids at its N-terminus and/or at its C-terminus and/or internally, preferably at its C-terminus.

A peptide fragment of the MATα2 peptide has preferably a deletion of up to 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 135, 140, 148, 150, or 160 amino acids at its N-terminus and/or at its C-terminus and/or internally, preferably at its C-terminus.

A peptide fragment of the MFα1 peptide has preferably a deletion of up to 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 135, 140, 148, 150, or 160 amino acids at its N-terminus and/or at its C-terminus and/or internally, preferably at its C-terminus.

Preferably, module (c) of the conjugate of the present invention comprises or consists of a peptide of the human COX2 protein (UniProt P35354; SEQ ID NO: 41). It is particularly preferred that module (c) of the conjugate of the present invention comprises or consists of a C-terminal peptide fragment of the human COX2 protein comprising or consisting of, preferably consisting of amino acids 504 through 604 (SEQ ID NO: 42) of human COX2. More preferably, module (c) of the conjugate of the present invention comprises or consists of a C-terminal peptide fragment of the human COX2 protein comprising or consisting of, preferably consisting of either amino acids 580 through 598 (SEQ ID NO: 43) or amino acids 580 through 604 (SEQ ID NO: 44) of human COX2.

In a particular preferred embodiment of the conjugate of the present invention, module (c) comprises, essentially consists or consists of a peptide comprising or consisting of the amino acid sequence NX₁SX₂X₃X₄X₅X₆X₇X₈X₉INPTX₁₀X₁₁X₁₂X₁₃ (SEQ ID NO: 45) of COX2, wherein X₁ is A, S or V; X₂ is S, A or T; X₃ is S or V; X₄ is R, H or N; X₅ is S or T; X₆ is G, R, T or A; X₇ is L, V or M; X₈ is D, N or E; X₉ is D or N; X₁₀ is V or L; X₁₁ is L or V; X₁₂ is L or I; and X₁₃ is K or N.

In a more preferred embodiment of the conjugate of the present invention, module (c) comprises, essentially consists of or consists of a peptide comprising or consisting of the amino acid sequence NASSSRSGLDDINPTVLLK (SEQ ID NO: 43); NASASHSRLDDINPTVLIK (SEQ ID NO: 46); or NASSSHSGLDDINPTVLLK (SEQ ID NO: 47) of COX2.

In a particular preferred embodiment of the conjugate of the present invention, module (c) comprises, essentially consists of or consists of a peptide comprising or consisting of the amino acid sequence NX₁SSX₂X₃SX₄X₅DDINPTVLLK (SEQ ID NO: 48), wherein X₁ is A, G or V, X₂ is S or A, X₃ is R, H or N, X₄ is G, R or A, X₅ is L or S.

In a more particularly preferred embodiment of the conjugate of the present invention, module (c) comprises, essentially consists of or consists of a peptide comprising or consisting of the amino acid sequence NASSSRSGLDDINPTVLLKERSTEL (SEQ ID NO: 44) of human COX2.

Preferably, module (c) of the conjugate of the present invention comprises, essentially consists of or consists of a peptide of the mouse IgM(μ) protein (Accession number CAA27326; SEQ ID NO: 49). It is particularly preferred that module (c) of the conjugate of the present invention comprises or consists of a C-terminal peptide fragment of the mouse IgM(μ) protein comprising or consisting of, preferably consisting of amino acids 421 through 455 (SEQ ID NO: 50) of mouse IgM(μ). More preferably, module (c) of the conjugate of the present invention comprises or consists of a C-terminal peptide fragment of the mouse IgM(μ) protein comprising or consisting of, preferably consisting of amino acids 436 through 455 (SEQ ID NO: 51) of mouse IgM(μ).

In a more preferred embodiment of the conjugate of the present invention, module (c) comprises, essentially consists of or consists of a peptide comprising or consisting of the amino acid sequence GKPTLYNVSLIMSDTGGTCY (SEQ ID NO: 51); GKPTLYNVSLVMSDTAGTCY (SEQ ID NO: 52); GKPTLYQVSLIMSDTGGTCY (SEQ ID NO: 53); or GKPTLYQVSLIM SDTGGTSY (SEQ ID NO: 54) of IgM(μ).

In an even more preferred embodiment of the conjugate of the present invention, module (c) comprises, essentially consists of or consists of a peptide comprising or consisting of the amino acid sequence EQKLISEEDLGKPTLYQVSLIMSDTGGTSY [SEQ ID NO: 226; human c-myc tagged-IgM(0].

Preferably, module (c) of the conjugate of the present invention comprises, essentially consists of or consists of a peptide of the human IgM(μ) protein (Accession number CAC20458; SEQ ID NO: 55). It is particularly preferred that module (c) of the conjugate of the present invention comprises or consists of a C-terminal peptide fragment of the human IgM(μ) protein comprising or consisting of, preferably consisting of amino acids 421 through 455 (SEQ ID NO: 56) of human IgM(μ). More preferably, module (c) of the conjugate of the present invention comprises or consists of a C-terminal peptide fragment of the human IgM(μ) protein comprising or consisting of, preferably consisting of amino acids 436 through 455 (SEQ ID NO: 52) of human IgM(μ).

In a particularly preferred embodiment of the conjugate of the present invention, module (c) comprises, essentially consists of or consists of a peptide comprising or consisting of the amino acid sequence GKPTLYX₁VSLX₂MSDTX₃GTX₄Y (SEQ ID NO: 57) of IgM(0, wherein X₁ is N or Q; X₂ is I or V; X₃ is G or A; and X₄ is C or S.

Preferably, module (c) of the conjugate of the present invention comprises, essentially consists of or consists of a peptide of the mouse Sgk1 protein (UniProt Q9WVC6; SEQ ID NO: 58). It is particularly preferred that module (c) of the conjugate of the present invention comprises, essentially consists of or consists of an N-terminal peptide fragment of the mouse Sgk1 protein comprising or consisting of, preferably consisting of amino acids 1 through 100 (SEQ ID NO: 59) of mouse Sgk1. Preferably, module (c) of the conjugate of the present invention comprises, essentially consists of or consists of an N-terminal peptide fragment of the mouse Sgk1 protein comprising or consisting of, preferably consisting of amino acids 1 through 60 (SEQ ID NO: 60) of mouse Sgk1 protein. Preferably, module (c) of the conjugate of the present invention comprises, essentially consists of or consists of an N-terminal peptide fragment of the mouse Sgk1 protein comprising or consisting of, preferably consisting of amino acids 1 through 33 (SEQ ID NO: 61) of mouse Sgk1 protein.

Preferably, module (c) of the conjugate of the present invention comprises, essentially consists of or consists of a peptide of the human Sgk1 protein (UniProt accession number O0014; SEQ ID NO: 62). It is particularly preferred that module (c) of the conjugate of the present invention comprises, essentially consists of or consists of an N-terminal peptide fragment of the human Sgk1 protein comprising or consisting of, preferably consisting of amino acids 1 through 100 (SEQ ID NO: 63) of human Sgk1. Preferably, module (c) of the conjugate of the present invention comprises, essentially consists of or consists of an N-terminal peptide fragment of the human Sgk1 protein comprising or consisting of preferably, consisting of amino acids 1 through 60 (SEQ ID NO: 64) of human Sgk1 protein. Preferably, module (c) of the conjugate of the present invention comprises, essentially consists of or consists of an N-terminal peptide fragment of the human Sgk1 protein comprising or consisting of, preferably consisting of amino acids 1 through 33 (SEQ ID NO: 65) of human Sgk1 protein. Preferably, module (c) of the conjugate of the present invention comprises, essentially consists of or consists of an N-terminal peptide fragment of the human Sgk1 protein comprising or consisting of, preferably consisting of amino acids 1 through 30 (SEQ ID NO: 66) of human Sgk1 protein.

In a particular preferred embodiment of the conjugate of the present invention, module (c) comprises, essentially consists of or consists of a peptide comprising the amino acid sequence MTX₁X₂X₃X₄EX₅X₆X₇X₈X₉X₁₀X₁₁LTYSX₁₂X₁₃RGX₁₄VAX₁₅LX₁₆AFMKQRX₁₇MGLNDFIQK X₁₈X₁₉X₂₀NX₂₁YACKHX₂₂EVQSX₂₃LX₂₄X₂₅ (SEQ ID NO: 67) of mouse Sgk1, wherein X₁ is V or I; X₂ is K or Q; X₃ is A or T; X₄ is X [X is zero (0) amino acid] or A; X₅ is A or T; X₆ is A or S; X₇ is R, K, G or V; X₈ is S, G or P; X₉ is T, P or A; X₁₀ is X or P; X₁₁ is X or D; X₁₂ is R or K; X₁₃ is M or T; X₁₄ is M or L; X₁₅ is I or N; X₁₆ is I or S; X₁₇ is R or K; X₁₈ is I or L; X₁₉ is A or S; X₂₀ is S, N, A or T; X₂₁ is T or S; X₂₂ is A, P or T; X₂₃ is I or Y; X₂₄ is K or N; and X₂₅ is M, I or L.

In a more preferred embodiment of the conjugate of the present invention, module (c) comprises, essentially consists of or consists of a peptide comprising the amino acid sequence MTVKAEAARSTLTYSRMRGMVAILIAFMKQRRMGLNDFIQKIASNTYACKHAEVQSIL KM of mouse Sgk1 (SEQ ID NO: 60); MTVKTEAAKGTLTYSRMRGMVAILIA FMKQRRMGLNDFIQKIANNSYACKHPEVQSILKI (SEQ ID NO: 64) of human Sgk1; MTVKTEAAKGTLTYSRMRGMVAILIAFMKQ (SEQ ID NO: 66) of human Sgk1; MTVKTEAARSTLTYSRMRGMVAILIAFMKQRRMGLNDFIQKLANNSYACKHPEVQSYL KI (SEQ ID NO: 68) of rat Sgk1 (also referred to as Igh6; Accession number AAI05826); MTVKTEAARGPLTYSRMRGMVAILIAFMKQRRMGLNDFIQKIANNSYACKHTEVQSIL KI (SEQ ID NO: 69) of rabbit Sgk1; MTVKAAEASGPALTYSKMRGMVAILIAFMKQRRM GLNDFIQKIATNSYACKHPEVQSILK (SEQ ID NO: 70) of chicken Sgk1; or MTIQTETSV SAPDLTYSKTRGLVANLSAFMKQRKMGLNDFIQKLSANSYACKHPEVQSIL (SEQ ID NO: 71) of zebrafish Sgk1.

In a more preferred embodiment of the conjugate of the present invention, module (c) comprises, essentially consists of or consists of a peptide comprising the amino acid sequence MTVKTEAAKGTLTYSRMRGMVAILIAFMKQ (SEQ ID NO: 66), MRGMVAILIAF MKQRRMGLNDFIQKIASNTYACKHAEVQSILKM (SEQ ID NO: 72); MRGMVAIL IAFMKQ (SEQ ID NO: 73); GMVAILIAF (SEQ ID NO: 74); MRGMVAILIAFM KQRRM (SEQ ID NO: 75), GMVAILI (SEQ ID NO: 76), or MRGMVAILIAFMKQRR MGLNDFIQKIANNSYACKHPEVQSILKI (SEQ ID NO: 77) of Sgk1, designated as an Sgk1 peptide fragment.

Preferably, module (c) of the conjugate of the present invention comprises, essentially consists of or consists of a peptide of the MATα2 peptide from yeast (NCBI RefSeq NP_{—}009868) (SEQ ID NO: 78). It is particularly preferred that module (c) of the conjugate of the present invention comprises or consists of an N-terminal peptide fragment of the MATα2 peptide from yeast comprising amino acids 1 through 100 (SEQ ID NO: 79). More preferably, module (c) of the conjugate of the present invention comprises, essentially consists of or consists of an N-terminal peptide fragment of the MATα2 protein from yeast comprising amino acids 1 through 62 (SEQ ID NO: 80; also referred to as Deg1 degradation signal) of MATα2.

In a particular preferred embodiment of the conjugate of the present invention, module (c) comprises, essentially consists of or consists of a peptide comprising the amino acid sequence MNKIPIKDLLNPQITDEFKSSILDINKKLFSICCNLPKLPESVTTEEEVELRDILX₁FLSRAN (SEQ ID NO: 81) of MATα2, wherein X₁ is G, V or L.

In a more preferred embodiment of the conjugate of the present invention, module (c) comprises, essentially consists of or consists of a peptide comprising the amino acid sequence MNKIPIKDLLNPQITDEFKSSILDINKKLFSICCNLPKLPESVTTEEEVELRDILGFLSRAN (SEQ ID NO: 80); MNKIPIKDLLNPQITDEFKSSILDINKKLFSICCNLPKLPESVTT EEEVELRDILVFLSRAN (SEQ ID NO: 82); or MNKIPIKDLLNPQITDEFKSSIL DINKKLFSICCNLPKLPESVTTEEEVELRDI LLFLSRAN (SEQ ID NO: 83) of MATα2.

In a more preferred embodiment of the conjugate of the present invention, module (c) comprises, essentially consists of or consists of a peptide comprising the amino acid sequence ITDEFKSSILDINKKLFSI (SEQ ID NO: 84); or ITDEFKSSILDINKKLFSICCNL PKLPESV (SEQ ID NO: 85) of MATα2, designated as a MATα2 peptide fragment.

Preferably, module (c) of the conjugate of the present invention comprises, essentially consists of or consists of the yeast MFα1 peptide (SEQ ID NO: 86 [9]; UniProt P01149; Accession numbers CAA25738; AAA88727).

In a particular preferred embodiment of the conjugate of the present invention, module (c) comprises, essentially consists of or consists of a peptide comprising the amino acid sequence MRFPSIFTAVLFAASSALAAPVX₁TTTEDETAQIPAEAVIGYLDLEGDFDVAVLPFSX₁STN NGLLFIX₁TTIASIAAKEEGVSLDKREAEAWHWLQLKPGQPMYKREAEAEAWHWLQLK PGQPMYKREADAEAWHWLQLKPGQPMYKREADAEAWHWLQLKPGQPMY (SEQ ID NO: 87) of MFα1, wherein X₁ is N or Q.

In a more preferred embodiment of the conjugate of the present invention, module (c) comprises, essentially consists of or consists of a peptide comprising the amino acid sequence MRFPSIFTAVLFAASSALAAPVQTTTEDETAQIPAEAVIGYLDLEGDFDVAVLPFSQSTN NGLLFIQTTIASIAAKEEGVSLDKREAEAWHWLQLKPGQPMYKREAEAEAWHWLQLKP GQPMYKREADAEAWHWLQLKPGQPMYKREADAEAWHWLQLKPGQPMY (SEQ ID NO: 88); MRFPSIFTAVLFAASSALAAPVNTTTEDETAQIPAEAVIGYLDLEGDFDV AVLPFSNSTNNGLLFINTTIASIAAKEEGVSLDKREAEAWHWLQLKPGQPMYKREAEAE AWHWLQLKPGQPMYKREADAEAWHWLQLKPGQPMYKREADAEAWHWLQLKPGQP MY (SEQ ID NO: 86); MRFPSIFTAVLFAASSALAAPVNTTTEDETAQIPAEAVIGYLD LEGDFDVAVLPFSNSTNNGLLFIQTTIASIAAKEEGVSLDKREAEAWHWLQLKPGQPMY KREAEAEAWHWLQLKPGQPMYKREADAEAWHWLQLKPGQPMYKREADAEAWHWL QLKPGQPMY (SEQ ID NO: 89); or MRFPSIFTAVLFAASSALAAPVQTTTEDET AQIPAEAVIGYLDLEGDFDVAVLPFSNSTNNGLLFINTTIASIAAKEEGVSLDKREAEAW HWLQLKPGQPMYKREAEAEAWHWLQLKPGQPMYKREADAEAWHWLQLKPGQPMY KREADAEAWHWLQLKPGQPMY (SEQ ID NO: 90) of MFα1.

Preferably, module (c) of the conjugate of the present invention comprises, essentially consists of or consists of a peptide of the yeast CPY protein (Accession number P52710; SEQ ID NO: 91).

In another preferred embodiment, a peptide fragment of the CPY protein has a deletion of up to 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 135, 140, 148, 150, 160, 170, 180, 190, 200, 220, 250, 270, 300, 350, 370, 400, 420, 450, 470, 500, 505, 510, 515, 520 amino acids at its N-terminus, at its C-terminus, and/or internally.

Preferably, module (c) of the conjugate of the present invention comprises, essentially consists of or consists of a protein or a peptide of a toxin protein. A peptide or peptide fragment of a toxin protein preferably has a deletion of up to 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 135, 140, 148, 150, 160, 170, 180, 190, 200, 220, 250, 251, 258, 259, 270, 300, 315, 319, 350, 370, 400, 420, 450, 470, 500, 505, 510, 515, 520, 541, amino acids at its N-terminus and/or at its C-terminus and/or internally.

In a preferred embodiment, module (c) of the conjugate of the present invention comprises, essentially consists of or consists of a toxin protein or peptide selected from the group consisting of a toxin protein or peptide having reduced or no toxicity, an A/B type toxin protein or peptide having reduced or no toxicity, an A/B₅ type toxin protein or peptide having reduced or no toxicity, a toxin subunit having reduced or no toxicity, a toxin domain having reduced or no toxicity, an A/B type toxin subunit having reduced or no toxicity, an A/B₅ type toxin subunit having reduced or no toxicity, a mutated toxin A-subunit having reduced or no toxicity, a non-toxic toxin A1-subunit, a mutated toxin A1-subunit having reduced or no toxicity, and a toxin B-subunit. Preferably, module (c) comprises a mutated ricin toxin A-subunit (RTA) having reduced or no toxicity, a mutated ricin toxin A1-subunit (RTA1) having reduced or no toxicity, a ricin toxin B-subunit (RTB), a protein or peptide from a recombinantly produced ricin toxin B-subunit (e.g., as described in WO2008/157263), mutated cholera toxin A-subunit (CTA) having reduced or no toxicity, a mutated cholera toxin A1-subunit (CTA1) having reduced or no toxicity, a cholera toxin B-subunit (CTB), a mutated Shiga toxin (ST) A-subunit (STA) having reduced or no toxicity, a mutated Stx1a Shiga toxin A-subunit having reduced or no toxicity, a mutated Stx1b (VT1b) Shiga toxin A-subunit having reduced or no toxicity, a mutated Stx1c (VT1c) Shiga toxin A-subunit having reduced or no toxicity, a mutated Stx1d (VT1d) Shiga toxin A-subunit having reduced or no toxicity, a mutated Stx2a (VT2a) Shiga toxin A-subunit having reduced or no toxicity, a mutated Stx2b (VT2b) Shiga toxin A-subunit having reduced or no toxicity, a mutated Stx2c (VT2c) Shiga toxin A-subunit having reduced or no toxicity, a mutated Stx2d (VT2d) Shiga toxin A-subunit having reduced or no toxicity, a mutated Stx2e (VT2e) Shiga toxin A-subunit having reduced or no toxicity, a mutated Stx2f (VT2f) Shiga toxin A-subunit having reduced or no toxicity, a mutated Stx2g (VT2g) Shiga toxin A-subunit having reduced or no toxicity, a mutated Shiga toxin A1-subunit (STA1) having reduced or no toxicity, a mutated Stx1a Shiga toxin A1-subunit having reduced or no toxicity, a mutated Stx1b (VT1b) Shiga toxin A1-subunit having reduced or no toxicity, a mutated Stx1c (VT1c) Shiga toxin A1-subunit having reduced or no toxicity, a mutated Stx1d (VT1d) Shiga toxin A1-subunit having reduced or no toxicity, a mutated Stx2a (VT2a) Shiga toxin A1-subunit having reduced or no toxicity, a mutated Stx2b (VT2b) Shiga toxin A1-subunit having reduced or no toxicity, a mutated Stx2c (VT2c) Shiga toxin A1-subunit having reduced or no toxicity, a mutated Stx2d (VT2d) Shiga toxin A1-subunit having reduced or no toxicity, a mutated Stx2e (VT2e) Shiga toxin A1-subunit having reduced or no toxicity, a mutated Stx2f (VT2f) Shiga toxin A1-subunit having reduced or no toxicity, and a mutated Stx2g (VT2g) Shiga toxin A1-subunit having reduced or no toxicity, a Shiga toxin A1-subunit peptide, an Stx1a Shiga toxin A1-subunit peptide, an Stx1b (VT1b) Shiga toxin A1-subunit peptide, an Stx1c (VT1c) Shiga toxin A1-subunit peptide, an Stx1d (VT1d) Shiga toxin A1-subunit peptide, an Stx2a (VT2a) Shiga toxin A1-subunit peptide, an Stx2b (VT2b) Shiga toxin A1-subunit peptide, an Stx2c (VT2c) Shiga toxin A1-subunit peptide, an Stx2d (VT2d) Shiga toxin A1-subunit peptide, an Stx2e (VT2e) Shiga toxin A1-subunit peptide, an Stx2f (VT2f) Shiga toxin A1-subunit peptide, an Stx2g (VT2g) Shiga toxin A1-subunit peptide, a Shiga toxin B-subunit (STB), an Stx1a Shiga toxin B-subunit, an Stx1b (VT1b) Shiga toxin B-subunit, an Stx1c (VT1c) Shiga toxin B-subunit, an Stx1d (VT1d) Shiga toxin B-subunit, an Stx2a (VT2a) Shiga toxin B-subunit, an Stx2b (VT2b) Shiga toxin B-subunit, an Stx2c (VT2c) Shiga toxin B-subunit, an Stx2d (VT2d) Shiga toxin B-subunit, an Stx2e (VT2e) Shiga toxin B-subunit, an Stx2f (VT2f) Shiga toxin B-subunit, an Stx2g (VT2g) Shiga toxin B-subunit, a Shiga toxin B-subunit peptide, an Stx1a Shiga toxin B-subunit peptide, an Stx1b (VT1b) Shiga toxin B-subunit peptide, an Stx1c (VT1c) Shiga toxin B-subunit peptide, an Stx1d (VT1d) Shiga toxin B-subunit peptide, an Stx2a (VT2a) Shiga toxin B-subunit peptide, an Stx2b (VT2b) Shiga toxin B-subunit peptide, an Stx2c (VT2c) Shiga toxin B-subunit peptide, an Stx2d (VT2d) Shiga toxin B-subunit peptide, an Stx2e (VT2e) Shiga toxin B-subunit peptide, an Stx2f (VT2f) Shiga toxin B-subunit peptide, an Stx2g (VT2g) Shiga toxin B-subunit peptide, a mutated Escherichia coli heat labile enterotoxin (LT) A-subunit (LT-A) having reduced or no toxicity, a mutated LT-IIa A-subunit having reduced or no toxicity, a mutated LT-IIa A-subunit peptide having reduced or no toxicity, a mutated LT-IIb A-subunit having reduced or no toxicity, an LT B-subunit (LT-B), an LT-IIa B-subunit, an LT-IIb B-subunit, a mutated Abrin-a A-subunit having reduced or no toxicity, a mutated Abrin-b A-subunit having reduced or no toxicity, a mutated Abrin-c A-subunit having reduced or no toxicity, a mutated Abrin-d A-subunit having reduced or no toxicity, a mutated Pertussis A-subunit having reduced or no toxicity, a Pertussis B-subunit, a mutated Modeccin A-subunit having reduced or no toxicity, a Modeccin B-subunit, a mutated Volkensin A-subunit having reduced or no toxicity, a Volkensin B-subunit, a mutated Viscumin A-subunit having reduced or no toxicity, a Viscumin B-subunit, a mutated Pseudomonas Exotoxin A having reduced or no toxicity, a Pseudomonas Exotoxin Domain II, a mutated Escherichia coli subtilase cytotoxin A-subunit having reduced or no toxicity, an Escherichia coli subtilase cytotoxin B-subunit, a mutated Cinnamomin I toxin A-subunit having reduced or no toxicity, a mutated Cinnamomin II toxin A-subunit having reduced or no toxicity, a mutated Cinnamomin III toxin A-subunit having reduced or no toxicity, a mutated Sambucus ribosome-inactivating protein A-subunit having reduced or no toxicity, a mutated ribosome-inactivating protein SNAI′ A-subunit having reduced or no toxicity, a mutated Ebulin 1 ribosome-inactivating protein (ebu1) A-subunit having reduced or no toxicity, a mutated type 2 ribosome-inactivating protein SNAIf A-subunit having reduced or no toxicity, a mutated lectin [Q41358 (Q41358_SAMNI)] A-subunit having reduced or no toxicity, a mutated ribosome-inactivating protein (AV1) A-subunit having reduced or no toxicity, a mutated type 2 ribosome-inactivating protein Nigrin 1 A-subunit having reduced or no toxicity, a mutated type 2 ribosome-inactivating protein Nigrin b A-subunit having reduced or no toxicity, a mutated Bodinierin toxin A-subunit having reduced or no toxicity, a mutated Porrectin toxin A-subunit having reduced or no toxicity, or a mutated cinphorin toxin A-subunit with reduced or no toxicity. Preferably, a toxin protein or peptide for use as a module (c) in a conjugate of the invention lacks a signal peptide.

In a particular embodiment, a conjugate of the present invention comprises a module (c) comprising, essentially consisting of, or consisting of a toxin protein or peptide, wherein the protein or peptide is preferably non-toxic or has reduced toxicity. Preferably, a conjugate of the present invention comprises a module (c) comprising, essentially consisting of, or consisting of a toxin protein or peptide that is non-toxic or a mutated toxin protein or peptide, wherein the mutated toxin protein or peptide comprises an amino acid deletion, substitution, or insertion that renders the mutated toxin protein or peptide to have reduced or abolished toxicity compared to the wild-type toxin protein or peptide.

In a preferred embodiment, module (c) comprises or consists of a non-toxic or reduced toxicity protein or peptide of ricin toxin A1-subunit (SEQ ID NO: 1; ricin toxin A comprising an R180H substitution). In another preferred embodiment, module (c) comprises or consists of a mutated ricin toxin A1-subunit having reduced or no toxicity, wherein the mutated ricin toxin A1-subunit comprises a G247W substitution, an S250P substitution, a G247Q substitution, a W246R substitution, an E212D substitution, an E212K substitution, an 1287R substitution (Frankel et al., Mol Cell Biol. 1989. 9(2):415-20), an R215Q substitution, an E212Q substitution, a Y115S substitution, a Y158S substitution (Kim and Robertus, Protein Eng. 1992 December; 5(8):775-9), a deletion of amino acids 110-115 (DVTNAY; Ricin-A110-115; May et al., EMBO J. 1989. 8(1):301-8), or a Y115AN111M double substitution (RiVax; Vitetta et al., Proc Natl Acad Sci U S A. 2006 Feb. 14; 103(7):2268-73. Epub 2006 Feb. 3), and wherein the numerical position of the mutated ricin toxin A1-subunit's amino acid substitution or deletion is based upon the Uniprot sequence P02879 that comprises the full length ricin amino acid sequence, including the signal peptide. Preferably, a mutated ricin toxin A1-subunit having reduced or no toxicity for use as a module (c) in a conjugate of the invention lacks a signal peptide.

In a preferred embodiment, module (c) comprises or consists of a non-toxic or reduced toxicity protein or peptide of cholera toxin A1-subunit (SEQ ID NO: 153; cholera toxin A). More preferably, module (c) comprises or consists of a mutated cholera toxin A1-subunit comprising or consisting of SEQ ID NO: 154 (six amino acid insertion APRPGP at position 1 that renders the mutant CT more than 10 fold less toxic than wild-type CT, see Sanchez et al., J Biol. Chem. 2002. 277(36):33369-77. Epub 2002 Jun. 27), SEQ ID NO: 155 (sixteen amino acid insertion ASRCAELCCNPACPAP at position 1 that renders the mutant CT more than 100 fold less toxic than wild-type CT, Ibid.), SEQ ID NO: 156 (twenty-three amino acid insertion ANSSNYCCELCCNPACTGCYPGP at position 1 that renders the mutant CT more than 1000 fold less toxic than wild-type CT, Ibid.), an E112K substitution (Yamamoto et al., J Exp Med. 1997. 185(7):1203-10), an S61F substitution (Ibid.), or an E29H substitution (Periwal et al., Vaccine 2003. 21(5-6):376-85 and Tebbey et al., Vaccine 2000. 18(24):2723-34). Additional sequence information can also be found at http://www.uniprotorg/blast/?about=P01555[19-212]. Preferably, the mutated cholera toxin A-subunit for use as a module (c) lacks a signal peptide.

In a preferred embodiment, module (c) comprises or consists of a non-toxic or reduced toxicity protein or peptide of Shiga toxin A1-subunit peptide comprising or consisting of an amino acid sequence according to SEQ ID NO: 157, SEQ ID NO: 158, SEQ ID NO: 232, SEQ ID NO: 233, SEQ ID NO: 234, SEQ ID NO: 235, SEQ ID NO: 236, SEQ ID NO: 237, SEQ ID NO: 238, SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO: 256, fragment thereof, or variant thereof. In a preferred embodiment, module (c) comprises or consists of a non-toxic or reduced toxicity Shiga A1 subunit peptide. Preferably, the Shiga A1 peptide comprises or consists of an amino acid sequence according to ISFGSINAILGSVALILNCHHHASRVAR (SEQ ID NO: 159), ISFGSINAILGSVALILNCHHH (SEQ ID NO: 160), ISFGSINAILGSVALIL (SEQ ID NO: 161), or a fragment or variant thereof.

In another preferred embodiment, module (c) comprises or consists of a mutated Stx1b (VT1b) A subunit having reduced or no toxicity, wherein the mutated Stx1b (VT1b) A subunit comprises an E189Q/R192L double substitution, an E189Q substitution, or an R192L substitution, and wherein the numerical position of the mutated Stx1b (VT1b) A subunit's amino acid substitution is based upon the Uniprot Q9S5J3 (Q9S5J3_ECOLX) sequence (SEQ ID NO: 306). These mutants have been characterized by Ohmura et al., 1993 (Microb Pathog. 15(3):169-76). Preferably, the mutated Stx1b (VT1b) A subunit for use as a module (c) lacks a signal peptide.

In another preferred embodiment, module (c) comprises or consists of a mutated Shiga toxin Stx2e (VT2e) A subunit having reduced or no toxicity, wherein the mutated Shigatoxin Stx2e (VT2e) A subunit comprises an E189Q/R192L double substitution, an E189Q substitution, or an R192L substitution, and wherein the numerical position of the mutated Shiga toxin Stx2e (VT2e) A subunit's amino acid substitution is based upon the Stx2e/VT2e: Uniprot A9ZMR8 (A9ZMR8_ECOLX) sequence; SEQ ID NO: 307). These mutants have been characterized by Cao et al., 1994 (Microbiol Immunol. 38(6):441-7).

In a preferred embodiment, module (c) comprises or consists of a non-toxic or reduced toxicity protein or peptide of E. coli heat-labile enterotoxin LT A-subunit [SEQ ID NO: 167 (LT A human strain) or SEQ ID NO: 168 (LT A porcine strain)].

In another preferred embodiment, module (c) comprises or consists of a mutated LT A-subunit having reduced or no toxicity, wherein the mutated LT A-subunit comprises a S81K substitution, an A90R substitution, an S81Y substitution, a deletion of amino acids 128-130, or an E130K substitution, and wherein the numerical position of the mutated LT A-subunit's amino acid substitution or deletion is indicated according to the reference sequence Uniprot sequence P43530 containing a signal peptide. While the reference sequence used here (i.e., Uniprot sequence P43530) to identify the location of these mutations in the LT A-subunit comprises a signal peptide, the mutated LT A-subunit protein or peptide for use as a module (c) of the invention preferably lacks this signal peptide. These mutants have been described by Pizza et al. J Exp Med. 1994. 180(6):2147-53; Giuliani et al., 1998. J Exp Med. 187(7):1123-32; Douce et al. Infect Immun. 1999. 67(9):4400-6; Park et al., Exp Mol. Med. 2000. 32(2):72-8; Park et al., Exp Mol. Med. 1999. 31(2):101-7; and Sanchez and Holmgren, 2008 (Cell Mol Life Sci., 65(9):1347-60).

In a preferred embodiment, module (c) comprises or consists of a non-toxic or reduced toxicity protein or peptide of E. coli heat-labile enterotoxin LT-IIa A-subunit (SEQ ID NO: 169; LT-IIa A). Preferably, module (c) of the conjugate of the present invention comprises or consists of a non-toxic or reduced toxicity peptide of LT-IIa A-subunit that comprises an amino acid sequence according to YQLAGFPSNFPAWREMPWSTFAPEQCVPNNK (SEQ ID NO: 170),

In another preferred embodiment, module (c) comprises or consists a non-toxic or reduced toxicity protein or peptide of E. coli heat-labile enterotoxin LT-IIb A-subunit (SEQ ID NO: 171; LT-IIb A).

Pertussis toxin A-subunit substitution and deletion mutants have been described in the art (see Loosmore et al., Infect Immun 1990. 58(11):3653-62). Preferably, a mutated pertussis toxin A-subunit of use in the present invention comprises a residual toxicity of 1% or less compared to the wild-type pertussis toxin A-subunit. More preferably, a mutated pertussis toxin A-subunit of use in the present invention comprises a residual toxicity of less than 0.01% compared to the wild-type pertussis toxin A-subunit. Even more preferably, a mutated pertussis toxin A-subunit of use in the present invention comprises no residual toxicity compared to the wild-type pertussis toxin A-subunit.

In a preferred embodiment, module (c) comprises or consists a non-toxic or reduced toxicity protein or peptide of pertussis toxin A-subunit (SEQ ID NO: 172; Pertussis toxin subunit 1 (=PTX 51); http://www.uniprot.org/uniprot/P04977; which comprises a signal peptide).

In another preferred embodiment, module (c) comprises or consists of a mutated pertussis toxin A-subunit having reduced or no toxicity, wherein the mutated pertussis toxin A-subunit comprises an R43 amino acid deletion, an R43K substitution, an R43H substitution, a five (5) amino acid deletion of R43 to R47, an R92E substitution, a W60A substitution, an H69A substitution, a C75A substitution, an E163 amino acid deletion, an E163G substitution, an E163Q substitution, an E163D substitution, an E163N substitution, an E163K substitution, an E163H substitution, an E163P substitution, an E163S substitution, an E163G/Y164A double substitution, an E163G/Y164F double substitution, a C75A/E163G double substitution, an R43K/E163G double substitution, an R43K/R92E/E163G triple substitution, or an R92E/E163G double substitution, wherein the numerical position of the amino acid deletion or substitution is indicated according to the reference sequence Uniprot sequence P04977. A particularly preferred mutant pertussis A-subunit protein or peptide comprises or contains an R43 amino acid deletion, an R43K substitution, an R43K/R92E/E163G triple substitution, or an R92E/E163G double substitution, wherein the numerical position of the amino acid deletion or substitution is indicated according to the reference sequence Uniprot sequence P04977. While the reference sequence used here (i.e., Uniprot sequence P04977) to identify the location of these mutations in the pertussis toxin A-subunit comprises a signal peptide, the mutated pertussis toxin A-subunit protein or peptide for use as a module (c) of the invention preferably lacks this signal peptide.

Preferably, a mutated E. coli subtilase cytotoxin A-subunit of use in the present invention comprises a residual toxicity of 1% or less compared to the wild-type E. coli subtilase cytotoxin A-subunit. More preferably, a mutated E. coli subtilase cytotoxin A-subunit of use in the present invention comprises a residual toxicity of 0.1% or less compared to the wild-type E. coli subtilase cytotoxin A-subunit. Even more preferably, a mutated E. coli subtilase cytotoxin A-subunit of use in the present invention comprises no residual toxicity compared to the wild-type E. coli subtilase cytotoxin A-subunit.

In a preferred embodiment, module (c) comprises or consists a non-toxic or reduced toxicity protein or peptide of an E. coli subtilase cytotoxin A-subunit comprising or consisting of an amino acid sequence selected from the group consisting of SEQ ID NO: 173, SEQ ID NO: 174, and SEQ ID NO: 175.

In another preferred embodiment, module (c) comprises or consists of a mutated E. coli subtilase cytotoxin A-subunit having reduced or no toxicity, wherein the mutated E. coli subtilase cytotoxin A-subunit comprises a S272A substitution, and wherein the numerical position of the mutated E. coli subtilase cytotoxin A-subunit's amino acid substitution is based upon the http://www.uniprot.org/uniprot/Q6EZC2 sequence. This mutant has been described by Paton et al., 2004. (J Exp Med. 2004. 200(1):35-46. Epub 2004 Jun. 28. Erratum in: J Exp Med. 2004. 200(11):1525. PMID: 15226357). Preferably, the mutated E. coli subtilase cytotoxin A-subunit for use as a module (c) lacks a signal peptide.

In a preferred embodiment, module (c) comprises or consists a non-toxic or reduced toxicity protein or peptide of an Abrin toxin A-subunit comprising or consisting of an amino acid sequence selected from the group consisting of amino acids 1-251 of SEQ ID NO: 135 (Abrin a toxin; http://www.uniprot.org/uniprot/P11140), amino acids 1-250 of SEQ ID NO: 136 (Abrin b toxin; http://www.uniprot.org/uniprot/Q06077), amino acids 35-285 of SEQ ID NO: 137 (Abrin c toxin; http://www.uniprot.org/uniprot/P28590), and amino acids 1-251 of SEQ ID NO: 138 (Abrin d toxin; http://www.uniprot.org/uniprot/Q06076).

In another preferred embodiment, module (c) comprises or consists of a mutated Abrin a toxin A-subunit having reduced or no toxicity, wherein the mutated Abrin A-subunit comprises an E164A/R167L double substitution, an E164A substitution, or an R167L substitution, and wherein the numerical position of the mutated Abrin a toxin A-subunit's amino acid substitution is based upon the Uniprot P11140 (ABRA_ABRPR) sequence. These mutants have been described by Hung et al., 1994. (Eur J. Biochem. 219(1-2):83-7). Preferably, the mutated Abrin a toxin A-subunit for use as a module (c) lacks a signal peptide.

In a preferred embodiment, module (c) comprises or consists a non-toxic or reduced toxicity protein or peptide of a volkensin toxin A-subunit comprising or consisting of an amino acid sequence comprising SEQ ID NO: 176 (Chambery et al., Eur J. Biochem. 2004. 271(1):108-17).

In a preferred embodiment, module (c) comprises or consists a non-toxic or reduced toxicity protein or peptide of a viscumin toxin A-subunit comprising or consisting of an amino acid sequence comprising SEQ ID NO: 177 (http://www.uniprot.org/uniprot/P81446).

In a preferred embodiment, module (c) comprises or consists a non-toxic or reduced toxicity protein or peptide of a Pseudomonas exotoxin A-subunit (http://www.uniprot.org/uniprot/P11439>sp|P11439|26-638 that lacks the signal peptide sequence) comprising or consisting of an amino acid sequence comprising amino acids selected from the group consisting of amino acids 1-613 of SEQ ID NO: 114 (exotoxin A) and amino acids 253-364 of SEQ ID NO: 114 (exotoxin II).

In another preferred embodiment, module (c) comprises or consists of a mutated Pseudomonas exotoxin A having reduced or no toxicity, wherein the mutated Pseudomonas exotoxin A comprises a D599C substitution or an E553D substitution [see Benhar et al., J Biol. Chem. 1994. 269(18):13398-404, and Douglas and Collier, J. Bacteriol. 1987. 169(11):4967-71, respectively and P11439 (TOXA_PSEAE)]. Preferably, the mutated Pseudomonas exotoxin A-subunit for use as a module (c) lacks a signal peptide.

In a preferred embodiment, module (c) comprises or consists a non-toxic or reduced toxicity protein or peptide of a cinnamomin A-subunit comprising or consisting of an amino acid sequence selected from the group consisting of SEQ ID NO: 178 (cinnamomin I A-subunit), SEQ ID NO: 179 (cinnamomin II A-subunit), and SEQ ID NO: 180 (cinnamomin III A-subunit).

In a preferred embodiment, module (c) comprises or consists a non-toxic or reduced toxicity protein or peptide of a Sambucus ribosome-inactivating protein or peptide, a ribosome-inactivating protein SNAI′ A-subunit (SEQ ID NO: 181; http://www.uniprot.org/uniprot/P93543), an Ebulin 1 ribosome-inactivating protein (ebu1) A-subunit (SEQ ID NO: 182; http://www.uniprot.org/uniprot/Q9AVR2), a type 2 ribosome-inactivating protein SNAIf A-subunit (SEQ ID NO: 183; http://www.uniprot.org/uniprot/O22415), a lectin [Q41358 (Q41358_SAMNI)] A-subunit (SEQ ID NO: 184; http://www.uniprot.org/uniprot/Q41358.html), a ribosome-inactivating protein (AV1) A-subunit (SEQ ID NO: 185; http://www.uniprot.org/uniprot/Q945S2), a type 2 ribosome-inactivating protein Nigrin 1 A-subunit (SEQ ID NO: 186; http://www.uniprot.org/uniprot/Q8GT32), or a type 2 ribosome-inactivating protein Nigrin b A-subunit (SEQ ID NO: 187; http://www.uniprot.org/uniprot/P33183).

In another particularly preferred embodiment, module (c) comprises, consists essentially, or consists of a toxin protein or peptide selected from the group consisting of a ricin toxin B-subunit protein or peptide comprising or consisting of an amino acid sequence according to SEQ ID NO: 115 or SEQ ID NO: 116, or a recombinantly produced ricin toxin B-subunit as described in WO2008/157263; a cholera toxin B-subunit protein or peptide comprising or consisting of an amino acid sequence according to SEQ ID NO: 117 or SEQ ID NO: 118; a Shiga toxin (Stx) B-subunit protein or peptide comprising or consisting of an amino acid sequence according to SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 227, SEQ ID NO: 228, SEQ ID NO: 229, SEQ ID NO: 230, SEQ ID NO: 231, fragment thereof, or variant thereof; an LT-B B-subunit protein or peptide comprising or consisting of an amino acid sequence according to SEQ ID NO: 130 or SEQ ID NO: 131; an LT-IIa B-subunit protein or peptide comprising or consisting of an amino acid sequence according to SEQ ID NO: 132; an LT-IIb B-subunit protein or peptide comprising or consisting of an amino acid sequence according to SEQ ID NO: 133; an abrin toxin B-subunit protein or peptide comprising or consisting of an amino acid sequence according to SEQ ID NO: 134, amino acids 262-528 of SEQ ID NO: 135 (Abrin a toxin), amino acids 261-527 of SEQ ID NO: 136 (Abrin b toxin), amino acids 296-562 of SEQ ID NO: 137 (Abrin c toxin), or amino acids 262-528 of SEQ ID NO: 138 (Abrin d toxin); a pertussis toxin B-subunit comprising or consisting of an S2 protein, an S3 protein, two S4 proteins, and an S5 protein, wherein the S2 protein comprises an amino acid sequence comprising SEQ ID NO: 139 (Pertussis toxin subunit 2 (PTX S2); http://www.uniprot.org/uniprot/P04978), the S3 protein comprises an amino acid sequence comprising SEQ ID NO: 140 (Pertussis toxin subunit 3 (PTX S3); http://www.uniprot.org/uniprot/P04979), each of the two S4 proteins comprise an amino acid sequence comprising SEQ ID NO: 141 (Pertussis toxin subunit 4 (PTX S4); http://www.uniprot.org/uniprot/P0A3R5), and the S5 protein comprises an amino acid sequence comprising SEQ ID NO: 142 (Pertussis toxin subunit 5 (PTX S5); http://www.uniprot.org/uniprot/P04981); an E. coli subtilase cytotoxin B-subunit comprising or consisting of an amino acid sequence of SEQ ID NO: 143, SEQ ID NO: 144, or SEQ ID NO: 145; a volkensin toxin B-subunit comprising or consisting of an amino acid sequence comprising SEQ ID NO: 146 (Chambery et al., Eur J. Biochem. 2004. 271(1):108-17); a viscumin B-subunit comprising or consisting of an amino acid sequence comprising SEQ ID NO: 147 (http://www.uniprot.org/uniprot/P81446); a tetanus toxin C-fragment comprising or consisting of an amino acid sequence comprising SEQ ID NO: 148 and SEQ ID NO: 149; a fragment thereof, and a variant thereof.

In another embodiment, module (c) of the conjugate of the present invention comprises or consists of a viral peptide that facilitates translocation from the ER to the cytosol. Preferably, said viral peptide is from a polyomavirus. More preferably, said viral peptide is from SV40, murine polyomavirus, BK virus, JC virus, KI virus, WU virus, and Merkel Cell polyomavirus. Even more preferably, said viral peptide is from SV40 or murine polyomavirus. Polyomaviruses (e.g., mPyV and SV40) have been shown to be recognized as misfolded proteins within the ER by the ER associated degradation machinery and are subsequently transported to the cytosol by ERAD [37]. Thus, a viral peptide, fragment or variant from SV40, murine polyomavirus, BK virus, JC virus, KI virus, WU virus, or Merkel Cell polyomavirus may be used as a module (c) in the conjugates of the present invention.

In another particularly preferred embodiment, module (c) comprises, consists essentially, or consists of an AChE protein or peptide comprising an amino acid sequence selected from the group consisting of DTLDEAERQWKAEFHRWSSYMVHWKNQFDHYSKQERCSDL (SEQ ID NO: 280, rat AchE peptide), DTLDEAERQWKAEFHRWSSYMVHWKNQFDHYS KQERSSDL (SEQ ID NO: 281, rat AchE peptide), ETIDEAERQWKTEFHRWSSYMMH WKNQFDQYSRHENCA EL (SEQ ID NO: 282, Torpedo californica AchE peptide), ETIDEAERQWKTEFHRWSSYM MHWKNQFDQYSRHENSAEL (SEQ ID NO: 283, Torpedo californica AchE peptide), ETIDEAERQWKTEFHRWSCYMMHWKNQFDQY SRHENCAEL (SEQ ID NO: 284, Torpedo californica AchE peptide), ETIDEAERQWKTEFHRWSCYMMHWKNQFDQYSRHENSAEL (SEQ ID NO: 285, Torpedo californica AchE peptide), ETIDEAERQWKTEFHRWSSYCMH WKNQFDQYSRHENCAEL (SEQ ID NO: 286, Torpedo californica AchE peptide), ETIDEAERQWKTEFHRWSSY CMHWKNQFDQYSRHENSAEL (SEQ ID NO: 287, Torpedo californica AchE peptide), ETIDEAERQWKTEFHRWSCYCMHWKNQFDQYSRHENCAEL (SEQ ID NO: 288, Torpedo californica AchE peptide), and ETIDEAERQWKTEFHRWSCYCMHWKNQFDQY SRHENSAEL (SEQ ID NO: 289, Torpedo californica AchE peptide). For more information on AChE, see Belbeoc'h et al., 2003. EMBO J. 22:3536-3545. and Belbeoc'h et al., 2004. Eur J. Biochem. 271:1476-1487.

In another preferred embodiment, module (c) comprises, consists essentially, or consists of an AChE peptide selected from the group consisting of DTLDEAERQWRAEFHRWSSYMVH WKNQFDHYSKQERX₁SDL, wherein X₁ is C or S (SEQ ID NO: 290), and ETIDEAERQWKTEFHRWSX₁YX₂MHWKNQFDQYSRHENX₃AEL, wherein X₁ is C or S; X₂ is C or M; X₃ is C or S (SEQ ID NO: 291).

In a preferred embodiment of the conjugate of the present invention, module (a), module (b) and/or module (c) also comprises a peptide comprising or consisting of the amino acid sequence EQKLISEEDL [SEQ ID NO: 305; human c-myc epitope tag]. One purpose for incorporating such an epitope tag into a module of the invention is to facilitate purification of that module during synthesis and the resulting tagged module-comprising conjugate using an anti-c-myc antibody. Another purpose for incorporating such an epitope tag into a module (a), module (b), and/or module (c) of the invention is to allow one of skill in the art to track the intracellular distribution and protein localization of the resulting tagged module-comprising conjugate using an anti-c-myc antibody. Preferably, a mouse anti-c-myc 1-9e10 antibody (Roche, catalog #11667149001) is used according to standard methods (see also Frieden et al., 2004. Chem. BioDivers., 1:930-938, Gottschling et al., 1998. Bioconjugate Chem., 9: 831-837, and Shapira et al., 2007. J. Cell Sci. 120:4377-4387) to purify and/or detect the c-myc epitope tagged module (c) and the resulting tagged module (c) comprising conjugate. One of skill in the art will recognize that other epitope tags may be used in place of the human c-myc epitope tag in the modules (c) and resulting conjugates of the invention, and that are then exploited for purification and/or intracellular detection/localization using an antibody that recognizes the substituted epitope tag.

One of ordinary skill in the art is well aware of methods for producing module (c) according to the present invention. For example, the module (c) may be chemically synthesized, e.g., by liquid phase or solid phase peptide synthesis, or the peptide may be genetically engineered using recombinant DNA techniques and a cellular expression system, such as bacteria, e.g., Escherichia coli, yeast cells, insect cells, mammalian cells, etc., or an in vitro expression system.

In a preferred embodiment, module (a) and module (b) are comprised in a single contiguous protein or peptide or are comprised within two separate domains or subunits of a protein or peptide, and is referred to herein as a [module (a)+module (b)] protein or peptide.

Preferably, the [module (a)+module (b)] protein or peptide comprises, consists essentially of, consists of or contains a mutated holo-toxin having reduced or no toxicity, preferably an AB₅ or AB type of holo-toxin (ab1), a non-toxic subunit of a toxin protein (ab2), a mutated subunit of a toxin protein having reduced or no toxicity (ab3), a mutated A-subunit of a toxin protein having reduced or no toxicity (ab4), a mutated A+B-subunit of a toxin protein having reduced or no toxicity (ab5), a mutated ricin holo-toxin having reduced or no toxicity (ab6), a non-toxic subunit of a ricin toxin protein (ab7), a mutated subunit of a ricin toxin protein having reduced or no toxicity (ab8), a mutated A-subunit of a ricin toxin protein having reduced or no toxicity (ab9), an A-subunit of a ricin toxin protein that comprises an R180H mutation (SEQ ID NO: 1) (ab10), a mutated A+B-subunit of a ricin toxin protein having reduced or no toxicity (ab11), a mutated Shiga holo-toxin having reduced or no toxicity (ab12), a non-toxic subunit of a Shiga toxin protein (ab13), a mutated subunit of a Shiga toxin protein having reduced or no toxicity (ab14), a mutated A-subunit of a Shiga toxin protein having reduced or no toxicity (ab15), a mutated A+B-subunit of a Shiga toxin protein having reduced or no toxicity (ab16), a mutated Stx1a holo-toxin having reduced or no toxicity (ab17), a non-toxic subunit of an Stx1a Shiga toxin protein (ab18), a mutated subunit of an Stx1a Shiga toxin protein having reduced or no toxicity (ab19), a mutated A-subunit of an Stx1a Shiga toxin protein having reduced or no toxicity (ab20), a mutated A+B-subunit of an Stx1a Shiga toxin protein having reduced or no toxicity (ab21), a mutated Stx1b holo-toxin having reduced or no toxicity (ab22), a non-toxic subunit of an Stx1b Shiga toxin protein (ab23), a mutated subunit of an Stx1b Shiga toxin protein having reduced or no toxicity (ab24), a mutated A-subunit of an Stx1b Shiga toxin protein having reduced or no toxicity (ab25), a mutated A+B-subunit of an Stx1b Shiga toxin protein having reduced or no toxicity (ab26), a mutated Stx1c holo-toxin having reduced or no toxicity (ab27), a non-toxic subunit of an Stx1c Shiga toxin protein (ab28), a mutated subunit of an Stx1c Shiga toxin protein having reduced or no toxicity (ab29), a mutated A-subunit of an Stx1c Shiga toxin protein having reduced or no toxicity (ab30), a mutated A+B-subunit of an Stx1c Shiga toxin protein having reduced or no toxicity (ab31), a mutated Stx1d holo-toxin having reduced or no toxicity (ab32), a non-toxic subunit of an Stx1d Shiga toxin protein (ab33), a mutated subunit of an Stx1d Shiga toxin protein having reduced or no toxicity (ab34), a mutated A-subunit of an Stx1d Shiga toxin protein having reduced or no toxicity (ab35), a mutated A+B-subunit of an Stx1d Shiga toxin protein having reduced or no toxicity (ab36), a mutated Stx2a holo-toxin having reduced or no toxicity (ab37), a non-toxic subunit of an Stx2a Shiga toxin protein (ab38), a mutated subunit of an Stx2a Shiga toxin protein having reduced or no toxicity (ab39), a mutated A-subunit of an Stx2a Shiga toxin protein having reduced or no toxicity (ab40), a mutated A+B-subunit of an Stx2a Shiga toxin protein having reduced or no toxicity (ab41), a mutated Stx2b holo-toxin having reduced or no toxicity (ab42), a non-toxic subunit of an Stx2b Shiga toxin protein (ab43), a mutated subunit of an Stx2b Shiga toxin protein having reduced or no toxicity (ab44), a mutated A-subunit of an Stx2b Shiga toxin protein having reduced or no toxicity (ab45), a mutated A+B-subunit of an Stx2b Shiga toxin protein having reduced or no toxicity (ab46), a mutated Stx2c holo-toxin having reduced or no toxicity (ab47), a non-toxic subunit of an Stx2c Shiga toxin protein (ab48), a mutated subunit of an Stx2c Shiga toxin protein having reduced or no toxicity (ab49), a mutated A-subunit of an Stx2c Shiga toxin protein having reduced or no toxicity (ab50), a mutated A+B-subunit of an Stx2c Shiga toxin protein having reduced or no toxicity (ab51), a mutated Stx2d holo-toxin having reduced or no toxicity (ab52), a non-toxic subunit of an Stx2d Shiga toxin protein (ab53), a mutated subunit of an Stx2d Shiga toxin protein having reduced or no toxicity (ab54), a mutated A-subunit of an Stx2d Shiga toxin protein having reduced or no toxicity (ab55), a mutated A+B-subunit of an Stx2d Shiga toxin protein having reduced or no toxicity (ab56), a mutated Stx2e holo-toxin having reduced or no toxicity (ab57), a non-toxic subunit of an Stx2e Shiga toxin protein (ab58), a mutated subunit of an Stx2e Shiga toxin protein having reduced or no toxicity (ab59), a mutated A-subunit of an Stx2e Shiga toxin protein having reduced or no toxicity (ab60), a mutated A+B-subunit of an Stx2e Shiga toxin protein having reduced or no toxicity (ab61), a mutated Stx2f holo-toxin having reduced or no toxicity (ab62), a non-toxic subunit of an Stx2f Shiga toxin protein (ab63), a mutated subunit of an Stx2f Shiga toxin protein having reduced or no toxicity (ab64), a mutated A-subunit of an Stx2f Shiga toxin protein having reduced or no toxicity (ab65), a mutated A+B-subunit of an Stx2f Shiga toxin protein having reduced or no toxicity (ab66), a mutated Stx2g holo-toxin having reduced or no toxicity (ab67), a non-toxic subunit of an Stx2g Shiga toxin protein (ab68), a mutated subunit of an Stx2g Shiga toxin protein having reduced or no toxicity (ab69), a mutated A-subunit of an Stx2g Shiga toxin protein having reduced or no toxicity (ab70), a mutated A+B-subunit of an Stx2g Shiga toxin protein having reduced or no toxicity (ab71). a mutated cholera holo-toxin having reduced or no toxicity (ab72), a non-toxic subunit of a cholera toxin protein (ab73), a mutated subunit of a cholera toxin protein having reduced or no toxicity (ab74), a mutated A-subunit of a cholera toxin protein having reduced or no toxicity (ab75), or an AMF (ab76).

Preferably when the [module (a)+module (b)] protein or peptide is a non-toxic Shiga holo-toxin, a Shiga holo-toxin having reduced toxicity, a non-toxic subunit of a Shiga toxin protein, a subunit of a Shiga toxin protein having reduced toxicity, a non-toxic A-subunit of a Shiga toxin protein, an A-subunit of a Shiga toxin protein having reduced toxicity, a non-toxic A+B-subunit of a Shiga toxin protein, or an A+B-subunit of a Shiga toxin protein having reduced toxicity, the [module (a)+module (b)] protein or peptide is from a Shiga toxin selected from the group consisting of Stx1a, Stx1b (VT1b), Stx 1c (VT1c), Stx 1d (VT1d), Stx2a (VT2a), Stx2b (VT2b), Stx2c (VT2c), Stx2d (VT2d), Stx2e (VT2e), Stx2f (VT2f) and Stx2g (VT2g).

In another preferred embodiment, module (b) and module (c) are comprised in a single contiguous protein or peptide, or are comprised within two separate domains or subunits of a protein, and is referred to herein as a [module (b)+module (c)] protein or peptide. Preferably, the [module (b)+module (c)] protein or peptide is selected from the group consisting of NASSSRSGLDDINPTVLLKERSTEL (CX1a; SEQ ID NO: 2), NASSSRSGLDDINPT VLLKAKDEL (CX2a; SEQ ID NO: 3), GKPTLYQVSLIMSDTGGTSYKDEL (SEQ ID NO: 4), a reduced toxicity or non-toxic cholera toxin A-subunit, a reduced toxicity of non-toxic cholera toxin A2-subunit (http://www.uniprot.org/blast/?about=P01555 [213-258]), a reduced toxicity or non-toxic LT A-subunit (http://www.uniprot.org/blast/?about=P43530[19-258] and http://www.uniprot.org/blast/?about=P06717[19-258]), a reduced toxicity or non-toxic LT-II A-subunit (http://www.uniprot.org/b last/? about=P13810[19-259]), a reduced toxicity or non-toxic Pseudomonas exotoxin A-subunit (also known as NAD-dependent ADP-ribosyltransferase; Wolf and Elsasser, Int J Med. Microbiol. 2009 March; 299(3):161-76. Epub 2008 Oct. 23), and an AChE protein or peptide comprising an amino acid sequence selected from the group consisting of DTLDEAERQWRAEFHRWSSYMVHWKNQFDHYSKQERKDEL (SEQ ID NO: 292), ETIDEAERQWKTEFHRWSSYMMHWKNQFDQYSRHENKDEL (SEQ ID NO: 293), ETIDEAERQWKTEFHRWSCYMMHWKNQFDQYSRHENKDEL (SEQ ID NO: 294), ETIDEAERQWKTEFHRWSSYCMHWKNQFDQYSRHENKDEL (SEQ ID NO: 295), ETIDEAERQWKTEFHRWSCYCMHWKNQFDQYSRHENKDEL (SEQ ID NO: 296), ETIDEAERQWKTEFHRWSSYMMHWKNQFKDEL (SEQ ID NO: 297), ETIDEAERQWK TEFHRWSCYMMHWKNQFKDEL (SEQ ID NO: 298), ETIDEAERQWKTEFHRWSSYCM HWKNQFKDEL (SEQ ID NO: 299), ETIDEAERQWKTEFHRWSCYCMHWKNQFKDEL (SEQ ID NO: 300), ETIDEAERQWKTEFHRWSSYMMHWKNQFDQYKDEL (SEQ ID NO: 301), ETIDEAERQWKTEFHRWSCYMMHWKNQFDQYKDEL (SEQ ID NO: 302), ET IDEA ERQWKTEFHRWSSYCMHWKNQFDQYKDEL (SEQ ID NO: 303), and ETIDEAERQ WKTEFHRWSCYCMHWKNQFDQYKDEL (SEQ ID NO: 304; mutated Torpedo californica).

In another preferred embodiment, module (a) and module (c) are comprised in a single contiguous protein or peptide, or are comprised within two separate domains or subunits of a protein, and is referred to herein as a [module (a)+module (c)] protein or peptide.

In another preferred embodiment, module (a), module (b), and module (c) are comprised in a single contiguous protein or peptide, or are comprised within at least two different domains or subunits of a protein, and is referred to herein as a [module (a)+module (b)+module (c)] protein or peptide. Preferably, the [module (a)+module (b)+module (c)] protein or peptide is selected from the group consisting of a holo-toxin having reduced or no toxicity, a toxin protein comprising a subunit having reduced or toxicity, a toxin protein comprising an A-subunit having reduced or no toxicity, a toxin protein comprising an A-subunit having reduced or no toxicity, a ricin holo-toxin having reduced or no toxicity, a ricin toxin protein comprising a subunit having reduced or no toxicity, a ricin toxin protein comprising an A-subunit having reduced or no toxicity, a ricin toxin protein comprising an A-subunit that comprises an R180H mutation (SEQ ID NO: 1), a ricin holo-toxin comprising an A-subunit having reduced or no toxicity, a cholera holo-toxin having reduced or no toxicity, a cholera toxin protein comprising a subunit having reduced or no toxicity, a cholera toxin protein comprising a subunit having reduced or no toxicity, a cholera toxin protein comprising an A-subunit having reduced or no toxicity, mutated subunit of a cholera toxin protein having reduced or no toxicity, a mutated A-subunit of a cholera toxin protein having reduced or no toxicity, a cholera holo-toxin comprising an A-subunit having reduced or no toxicity, a Shiga holo-toxin having reduced or no toxicity, a Shiga toxin protein comprising a subunit having reduced or no toxicity, a Shiga toxin protein comprising an A-subunit having reduced or no toxicity, a Pseudomonas exotoxin A holo-toxin having reduced or no toxicity, a Pseudomonas exotoxin A protein having reduced or no toxicity, a hybrid toxin having reduced or no toxicity and comprising a mutated A-subunit of a first AB toxin and a B-subunit of a second and different AB toxin, a hybrid toxin having reduced or no toxicity and comprising a mutated A1-subunit of a first AB₅ toxin and a B-subunit of a second and different AB₅ toxin, a hybrid ricin-abrin toxin having reduced or no toxicity, a hybrid ricin-modeccin toxin having reduced or no toxicity, a hybrid ricin-viscumin toxin having reduced or no toxicity, a hybrid ricin-volkensin toxin having reduced or no toxicity, a hybrid abrin-modeccin toxin having reduced or no toxicity, a hybrid abrin-viscumin toxin having reduced or no toxicity, a hybrid abrin-volkensin toxin having reduced or no toxicity, a hybrid modeccin-viscumin toxin having reduced or no toxicity, a hybrid modeccin-volkensin toxin having reduced or no toxicity, a hybrid viscumin-volkensin toxin having reduced or no toxicity, a hybrid LT-cholera toxin having reduced or no toxicity, a hybrid cholera-Shiga toxin having reduced or no toxicity, a hybrid cholera-pertussis toxin having reduced or no toxicity, a hybrid Shiga-Shiga toxin having reduced or no toxicity, a hybrid Shiga-LT toxin having reduced or no toxicity, a hybrid Shiga-pertussis toxin having reduced or no toxicity, and a hybrid LT-pertussis toxin having reduced or no toxicity. Preferably when the [module (a)+module (b)+module (c)] protein or peptide is a Shiga holo-toxin having reduced or no toxicity, a Shiga toxin protein comprising a subunit having reduced or no toxicity, a Shiga toxin protein comprising an A-subunit having reduced or no toxicity, a hybrid cholera-Shiga toxin having reduced or no toxicity, a hybrid Shiga-Shiga toxin having reduced or no toxicity, a hybrid Shiga-LT toxin having reduced or no toxicity, or a hybrid Shiga-pertussis toxin having reduced or no toxicity, the Shiga toxin protein or peptide portion of the [module (a)+module (b)+module (c)] protein or peptide is from a Shiga toxin selected from the group consisting of Stx1a, Stx1b (VT1b), Stx1c (VT1c), Stx1d (VT1d), Stx2a (VT2a), Stx2b (VT2b), Stx2c (VT2c), Stx2d (VT2d), Stx2e (VT2e), Stx2f (VT2f) and Stx2g (VT2g). Preferably when the [module (a)+module (b)+module (c)] protein or peptide is a hybrid Shiga-Shiga toxin having reduced or no toxicity, the hybrid Shiga-Shiga toxin having reduced or no toxicity is a hybrid of two different Shiga toxins selected from the group consisting of Stx1a, Stx1b (VT1b), Stx1c (VT1c), Stx1d (VT1d), Stx2a (VT2a), Stx2b (VT2b), Stx2c (VT2c), Stx2d (VT2d), Stx2e (VT2e), Stx2f (VT2f) and Stx2g (VT2g).

In another embodiment, a [module (a)+module (b)+module (c)] protein or peptide of the conjugate of the present invention comprises or consists of a reduced toxicity or non-toxic hybrid toxin protein or peptide. Preferably, the reduced toxicity or non-toxic hybrid toxin protein or peptide comprises an A-subunit and a B-subunit from at least two different toxins. In the case of AB toxins, an A-subunit from one AB toxin is combined with a B-subunit from a second AB toxin to result in a hybrid AB toxin protein or peptide. Preferable AB toxins of use in the conjugates of the present invention include ricin, abrins, modeccin, viscumin, volkensin, and the like. Alternatively, a reduced toxicity or non-toxic A-subunit from one AB₅ toxin is combined with a B₅-subunit from a second AB₅ toxin to result in a hybrid AB₅ toxin protein or peptide. Preferable AB₅ toxins of use in the conjugates of the present invention include cholera toxin, Shiga toxins, E. coli heat-labile enterotoxins, pertussis toxin, and the like. Preferably, the hybrid AB₅ toxin protein or peptide comprises a non-toxic A2-subunit and B-subunit pentamer (B₅) from one AB₅ toxin and a reduced toxicity or non-toxic A1-subunit from a second AB₅ toxin, e.g., an A1(LTI) having reduced or no toxicity+an A2(CTx)+B5(CTx) hybrid toxin protein. Preferably the reduced toxicity or non-toxic A1-subunit of the hybrid toxin protein or peptide comprises a mutation that results in reduced or no toxicity, e.g., a mutated A1(LTI) having reduced or no toxicity+an A2(CTx)+B5(CTx) hybrid toxin protein.

Thus, a particularly preferred [module (a)+module (b)+module (c)] protein or peptide of the conjugate of the present invention comprises or consists of a hybrid AB toxin with reduced or no toxicity, a hybrid ricin A-subunit (RTA)-abrin B-subunit (AB-B) toxin with reduced or no toxicity, a hybrid abrin A-subunit (AB-A)-ricin B-subunit (RTB) with reduced or no toxicity, a hybrid AB₅ toxin with reduced or no toxicity, a hybrid LT-CT toxin with reduced or no toxicity, a hybrid A1(LT1)-A2(CT)-B₅(CT) toxin with reduced or no toxicity, a hybrid ST-ST toxin with reduced or no toxicity, or a hybrid A l(ST)-A2(ST)-B₅(ST) toxin with reduced or no toxicity. Preferably when the [module (a)+module (b)+module (c)] protein or peptide is a hybrid ST-ST toxin with reduced or no toxicity or a hybrid A l(ST)-A2(ST)-B₅(ST) toxin with reduced or no toxicity, the hybrid ST-ST toxin with reduced or no toxicity or the hybrid A1(ST)-A2(ST)-B₅(ST) toxin with reduced or no toxicity is a hybrid of at least two different Shiga toxins selected from the group consisting of Stx1a, Stx1b (VT1b), Stx1c (VT1c), Stx1d (VT1d), Stx2a (VT2a), Stx2b (VT2b), Stx2c (VT2c), Stx2d (VT2d), Stx2e (VT2e), Stx2f (VT2f) and Stx2g (VT2g).

Within the context of the present invention, the “at least one module (a), at least one module (b), and at least one module (c)” is also defined as a “delivery carrier” of the invention. Preferably, the delivery carrier comprises at least one module (a), at least one module (b), and at least one module (c), wherein the at least one module (a), the at least one module (b), and the at least one module (c) are linked to each other in any arrangement. More preferably, the delivery carrier of the present invention comprises any of the module combinations designated below as K1 to K4918, either alone or in combination with any other module or compound (d) according to the invention.

The conjugate of the present invention comprises at least one compound (d), wherein compound (d) is preferably a nucleic acid, a peptide, a protein, a pharmaceutical, a cytotoxic agent, a radioactive agent, or another therapeutic or diagnostic moiety.

In a preferred embodiment, compound (d) is a protein or peptide that enhances the effectiveness or efficiency of the delivery system (DARE™) or conjugates of the present invention. These preferred compound (d) proteins and peptides are referred to herein as “DARE enhancer” proteins and peptides and include but are not limited to Derlin-1 (Degradation in endoplasmic reticulum protein 1), Derlin-2, Derlin-3, KDEL receptor, ER oxidase (ERO1), and any protein involved in ERAD, such as Sec61 complex, BIP (also known as GRP78), HSP70 and HSC70, ERDJ1-5, p58, HDJ1-2, HSJ1, Cys string protein, SIL1, GRP170, BAP, BAG1-2, HSPBP1, HSP110, alpha-Crystallin, GRP94, HSP90, Calnexin, Calreticulin, Protein disulfide isomerase (PDI), ERP29, ERP57, ERP72, ERDJS, EDEM1-3, OS9, XTP3-B, HERP, HRD1, HERP, VIMP, BAP31, SVIP, and the like (see also Vembar and Brodsky, Nat Rev Mol Cell Biol. 2008 December; 9(12):944-57. Epub 2008 Nov. 12).

In a preferred embodiment, compound (d) is a nucleic acid. Preferably, the nucleic acid is a single stranded or double stranded DNA, a single stranded or double stranded RNA, an siRNA, a tRNA, an mRNA, a micro RNA (miRNA), a small nuclear RNA (snRNA), a small hairpin RNA (shRNA), a morpholino modified iRNA (for example, as described in US2010/0076056 and U.S. Pat. No. 7,745,608), a zippered inhibitory RNA (ziRNA, as described in WO 2009/074076), an anti-gene RNA (agRNA, for example [44]), or the like.

Preferably, the conjugate of the present invention is configured such that it comprises RTB-siRNA, RTB linked to an siRNA via a lysine linkage (for example, see FIG. 4), RTB linked to an siRNA via a cysteine linkage (for example, see FIG. 5), RTB-COX2 peptide-siRNA [for example, see FIGS. 6 (A) and (B)], RTB-COX2 peptide-AKDEL peptide-siRNA (for example, see FIG. 7), RTB-AKDEL peptide-siRNA (for example, see FIG. 8), RTB-Sgk1 peptide-AKDEL peptide-siRNA (for example, see FIG. 9), TfR peptide-COX2 peptide-AKDEL peptide-siRNA [for example, see FIGS. 10(A) and (B)], Sgk1 peptide-TfR peptide-AKDEL peptide-siRNA (for example, see FIG. 11), TfR peptide-AKDEL peptide-IgM(p) peptide-siRNA (for example, see FIG. 12), TfR peptide-IgM(p) peptide-AKDEL peptide-siRNA (for example, see FIG. 13), RTB-COX2 peptide-AKDEL peptide-2 siRNAs (for example, see FIG. 14), AMF-COX2STEL-siRNA (for example, see FIG. 22), AMF-MYCIGM_{[}]-siRNA (for example, see FIG. 23), CTB-COX₂STEL-siRNA (for example, see FIG. 24), CTB-mycIgMu-siRNA (for example, see FIG. 25), CTB-(—COX2STEL)-(-siRNA) (for example, see FIG. 26), CTB-(-mycIgMu)-(-siRNA) (for example, see FIG. 27), CTB-00X₂STEL-siRNA, wherein the CTB has residual reduced SPDP (for example, see FIG. 28), and CTB-MYCIgMu-siRNA, wherein the CTB has residual reduced SPDP (for example, see FIG. 29), any of the module combinations designated below as K1 to K20609 in combination with any siRNA according to the invention

Preferably, the conjugate of the present invention comprises a configuration as depicted in FIG. 4, FIG. 5, FIG. 6(A), FIG. 6(B), FIG. 7, FIG. 8, FIG. 9, FIG. 10(A), FIG. 10(B), FIG. 11, FIG. 12, FIG. 13, FIG. 14, FIG. 22, FIG. 23, FIG. 24, FIG. 25, FIG. 26, FIG. 27, FIG. 28, or FIG. 29.

As stated earlier, there is often a problem with delivering a nucleic acid molecule into a cell. The use of the conjugate of the present invention provides a suitable delivery system of delivering nucleic acid molecules into a cell, preferably into the cytoplasm of a cell. The nucleic acid molecules delivered by the conjugate of the present invention may be used, for example, to achieve targeted gene silencing in a wide range of experimental systems from plants to human cells. Preferably, the nucleic acid molecules delivered by the conjugate of the present invention are therapeutic nucleic acid molecules that may be used, for example, to achieve targeted gene silencing in an organism, wherein the organism is a mammal, preferably a human.

RNAi, or RNA-mediated interference, is a method of choice for achieving targeted gene silencing in a wide range of experimental systems from plants to human cells. Following introduction of siRNA or miRNA into the cell cytoplasm, these double-stranded RNA constructs can bind to a protein termed RISC. The sense strand of the siRNA or miRNA is displaced from the RISC complex providing a template within RISC that can recognize and bind mRNA with a complementary sequence to that of the bound siRNA or miRNA. Having bound the complementary mRNA, the RISC complex cleaves the mRNA and releases the cleaved strands. RNAi can provide down-regulation of specific proteins by targeting specific destruction of the corresponding mRNA that encodes for protein synthesis.

In a preferred embodiment, a conjugate of the present invention comprises a compound (d) that is an siRNA. In a more preferred embodiment, a conjugate of the present invention comprises at least 2 compounds (d) that are siRNAs. Preferably, the conjugate comprises at least 2-20 siRNAs, i.e., at least 2, 3, 4, 5, 6, 7, 8, 9, 10, 11, 12, 13, 14, 15, 16, 17, 18, 19, or 20 siRNAs. In a preferred embodiment, a conjugate of the present invention comprises at least 2-10 siRNAs. In another preferred embodiment, a conjugate of the present invention comprises 2-10, i.e., 1, 2, 3, 4, 5, 6, 7, 8, 9, or 10 siRNAs. Within certain preferred embodiments of the invention, it may be necessary to neutralize the charge of the at least 2-20 siRNAs comprised within a conjugate of the present invention using methods available in the art.

As mentioned above, a preferred conjugate of the present invention comprises at least 2 compounds (d). In a preferred embodiment, the conjugate comprises at least two compounds (d), wherein the first of the at least 2 compounds (d) is an siRNA, and the second of the at least 2 compounds (d) is a RISC component. In this preferred embodiment, co-delivery of at least one targeted siRNA and at least one RISC component as compounds (d) in a conjugate of the present invention, is useful to enhance the efficiency of RNAi in a target cell, particularly in target cells in which the RNAi machinery is limited, either endogenously or as a result of when multiple siRNAs/conjugate are delivered to the target cells.

The term “RISC component” means any protein or peptide that is a component or an associated protein of a RISC complex. Examples of RISC components for use in the conjugates of the present invention include but are not limited to Dicer (e.g., Dicer-1, Dicer-2, and the like), Argonaute family proteins (e.g., Argonaute 2, and the like), transactivating response RNA-binding protein (TRBP), double stranded RNA binding domain proteins and peptides (e.g., R2D2, R3D1, and the like), protein activator of protein kinase R(PACT), Argonaute-related proteins (e.g., Piwi and the like), helicases, and nucleases.

Antisense constructs can also inhibit mRNA translation into protein. Antisense constructs are single stranded oligonucleotides and are non-coding. These single stranded oligonucleotides have a complementary sequence to that of the target protein mRNA and can bind to the mRNA by Watson-Crick base pairing. This binding either prevents translation of the target mRNA and/or triggers RNase H degradation of the mRNA transcripts, depending upon the type of chemical modifications used in the antisense construct. Consequently, antisense oligonucleotides have tremendous potential for specificity of action (i.e., down-regulation of a specific disease-related protein). To date, these compounds have shown promise in several in vitro and in vivo models, including models of inflammatory disease, cancer, and HIV [reviewed in 45]. Antisense can also affect cellular activity by hybridizing specifically with chromosomal DNA.

Coding nucleic acid molecules can also be used. Coding nucleic acid molecules (e.g. DNA) designed to function as a substrate for relevant RNA polymerases or ribosomes to directly drive transcription or translation of encoded product contained within its sequence, typically contain an open reading frame and appropriate regulatory motifs, e.g. promoter sequences, start, stop, poly A signals, and the like.

Preferably, the nucleic acid of the conjugate of the present invention is chemically modified. Nucleic acids comprising single or multiple modifications of the phosphodiester backbone or of the backbone, the sugar, and/or the nucleobases are preferred for use in the present invention. These chemically modifications have the positive effect that they stabilize the nucleic acid and have little impact on their activity. These chemical modifications can further prevent unwanted side effects of the nucleic acid like immune reactions via TLR's and/or the interferon pathway, or expression regulation of unintended target genes [i.e., Off Target Effects (OTEs)].

Preferred modifications of the phosphodiester backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thiono-alkylphosphonates, thionoalkylphosphotriesters, phosphoroselenate, methylphosphonate, or O-alkyl phosphotriester linkages, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′.

Modified nucleobases include other synthetic and natural nucleobases such as 5-methylcytosine (5-Me-C or m5C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-aza uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine, and 3-deazaguanine and 3-deazaadenine.

Modified nucleic acids may also contain one or more substituted sugar moieties. For example, the invention includes nucleic acids that comprise one of the following at the 2′ position: OH; F; O-, S-, or N-alkyl, O-alkyl-O-alkyl, O-, S-, or N-alkenyl, or O-, S- or N-alkynyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)₂ON(CH₃)₂, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred modified nucleic acids comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Further sugar modifications include, e.g. 2′-O-methyl, a locked nucleic acid (LNA), 2′-F, an unlocked nucleic acid (UNA), etc. Preferred backbone modifications include, e.g. peptide nucleic acid (PNA), morpholino, etc.

A “locked nucleic acid” (LNA) according to the present invention, often referred to as inaccessible RNA, is a modified RNA nucleotide. The ribose moiety of an LNA nucleotide is modified with an extra bridge connecting the 2′ oxygen and 4′ carbon. The bridge “locks” the ribose in the 3′-endo (North) conformation.

An “unlocked nucleic acid” (UNA) according to the present invention is comprised of monomers that are acyclic derivatives of RNA that lack the C2′-C3′-bond of the ribose ring of RNA.

A “peptide nucleic acid” (PNA) according to the present invention has a backbone composed of repeating N-(2-aminoethyl)-glycine units linked by peptide bonds.

In another preferred embodiment, compound (d) is a protein or a peptide. Proteins and peptides that may be delivered preferably include single chain antibodies, kinases, phosphatases, nucleases, inflammatory proteins, anti-infectious proteins, anti-angiogenic proteins, anti-inflammatory proteins, or any other protein or peptide or small molecule that is desired to be delivered to a cell, preferably to the cytosol of a cell.

Preferably, a compound (d) comprising a protein or peptide is coupled to modules (a), (b), and (c) via a disulfide linkage, in similar fashion as an siRNA described above and within the Examples, whereby the protein or peptide is cleaved from the delivery modules of the conjugate upon reaching the cytoplasm and is able to perform its intended function within the target cell. In an alternative preferred embodiment, an enzymatic cleavage site, as described above, is preferably present within the conjugate to enable release of compound (d) at the target cell's desired compartment, organelle or cytosol, or to separate compound (d) from the conjugate modules. In a particularly preferred embodiment, a conjugate of the present invention comprises a compound (d) comprising a protein or peptide, wherein the compound (d) is coupled to modules (a), (b), and (c) via a disulfide linkage, and wherein an enzymatic cleavage site is positioned within the conjugate, that when cleaved by an enzyme, releases compound (d) from the conjugate.

In a preferred embodiment, the compound (d) is an antigen that is desired to be delivered to the cytosol. Within this embodiment, an enzymatic cleavage site is preferably present within the conjugate to enable release of the antigen in the target cell's cytosol. Preferably, when compound (d) is an antigen, module (a) comprises a B-subunit of a toxin or a fragment or variant thereof. Preferably, the B-subunit of a toxin is a ricin B-subunit (RTB) or a Shiga toxin B-subunit selected from the groupd consisting of an Stx1a Shiga toxin B-subunit, an Stx1b (VT1b) Shiga toxin B-subunit, an Stx1c (VT1c) Shiga toxin B-subunit, an Stx1d (VT1d) Shiga toxin B-subunit, an Stx2a (VT2a) Shiga toxin B-subunit, an Stx2b (VT2b) Shiga toxin B-subunit, an Stx2c (VT2c) Shiga toxin B-subunit, an Stx2d (VT2d) Shiga toxin B-subunit, an Stx2e (VT2e) Shiga toxin B-subunit, an Stx2f (VT2f) Shiga toxin B-subunit, and an Stx2g (VT2g) Shiga toxin B-subunit. Such B-subunit toxin-antigen comprising conjugates of the invention are useful as vaccines to immunize an animal, preferably a mammal, more preferably a human (see for example, [46, 47].

In another preferred embodiment, module (a) comprises a non-toxic holo-toxin, wherein the non-toxic holo-toxin is preferably a non-toxic ricin holo-toxin or a non-toxic Shiga holo-toxin selected from the group consisting of a non-toxic Stx1a Shiga holo-toxin, a non-toxic Stx1b (VT1b) Shiga holo-toxin, a non-toxic Stx1c (VT1c) Shiga holo-toxin, a non-toxic Stx1d (VT1d) Shiga holo-toxin, a non-toxic Stx2a (VT2a) Shiga holo-toxin, a non-toxic Stx2b (VT2b) Shiga holo-toxin, a non-toxic Stx2c (VT2c) Shiga holo-toxin, a non-toxic Stx2d (VT2d) Shiga holo-toxin, a non-toxic Stx2e (VT2e) Shiga holo-toxin, a non-toxic Stx2f (VT2f) Shiga holo-toxin, and a non-toxic Stx2g (VT2g) Shiga holo-toxin. Preferably, the non-toxic holo-toxin comprises an A-subunit, wherein the A-subunit comprises a mutation that eliminates or greatly reduces the toxicity of the holo-toxin. A non-toxic holo-toxin comprising a mutated A-subunit is able to provide the functionalities of modules (a), (b) and (c) of a conjugate of the invention. Preferably, the non-toxic holo-toxin is a non-toxic ricin holo-toxin, wherein ricin A-subunit comprises an R→H substitution mutation at amino acid 180 (an R180H mutation) of ricin A-subunit (SEQ ID NO: 1).

Preferably, the functionality of modules (a) and (b) are comprised within the non-toxic holo-toxin B-subunit and the functionality of module (c) is comprised within the non-toxic holo-toxin mutated A-subunit. Preferably, compound (d) is an antigen coupled to the mutated A-subunit of the non-toxic holo-toxin that comprises module (a), module (b), and module (c). Such mutated A-subunit comprising holo-toxin-antigen comprising conjugates of the invention are useful as vaccines to immunize an animal, preferably a mammal, more preferably a human (see for example, [48]).

Antigens that are contemplated to be delivered using the present invention include but are not limited to NSP4, Influenza nucleoprotein NP, LCMV glycoprotein 1, hTRT, CYFRA 21-1, p53, ras, β-catenin, CDK4, CDC27, a actinin-4, tyrosinase, TRP1/gp75, TRP2, gp100, Melan-A/MART1, gangliosides, PSMA, HER2, WT1, EphA3, EGFR, CD20, MAGE, BAGE, GAGE, NY-ESO-1, and Survivin.

In another preferred embodiment, compound (d) comprises a protein or peptide, wherein the protein or peptide has been engineered to avoid or greatly reduce the risk of degradation by the target cell's proteasome. Preferably, compound (d) comprises a protein or peptide whose site of activity is either in the cytosol or in one of the target cell's compartments or organelles through which the conjugates of the present invention travel. Within this embodiment, an enzymatic cleavage site is preferably present within the conjugate to enable release of the protein or peptide at the target cell's desired compartment, organelle or cytosol.

In another embodiment of the present invention, small molecules (i.e., drugs), therapeutic molecules, diagnostic/imaging molecules, and the like that are desired to be delivered to either the cytosol or one of the target cell's compartments or organelles through which the conjugates of the present invention travel of a particular cell. Within this embodiment, an enzymatic cleavage site, as described above, is preferably present within the conjugate to enable release of the small molecule, therapeutic molecule, diagnostic molecule, or the like at the target cell's desired compartment, organelle or cytosol.

Small molecules that are contemplated to be delivered using the present invention include but are not limited to tamoxifen, dexamethasone, taxol, paclitaxel, cisplatin, oxaliplatin, and carboplatin.

Therapeutic molecules that are contemplated to be delivered using the present invention include but are not limited to antibodies, antibody fragments, peptides, peptoids, and decoy oligonucleotides.

Diagnostic or imaging molecules that are contemplated to be delivered using the present invention include but are not limited to Herpes simplex virus thymidine kinase (HSV1-TK, i.e., for tumor cell diagnostics/imaging), fluorochromes, quantum dots, (super-)(para-) magnetic nanoparticles, labelled antibodies, labelled antibody fragments, molecular beacons, biosensors (e.g. carbonic anhydrase), oligopeptide-based probes for detection of protease activity, peptide-based fluorescent sensors of protein kinase activity, radioactively-labeled metabolites, and D2R.

Tumor suppressor proteins and peptides that may be delivered according to the present invention include but are not limited to p53, p21, p15, BRCA1, BRCA2, IRF-1, PTEN, RB, APC, DCC, NF-1, NF-2, WT-1, MEN I, MEN-II, zacl, p73, VHL, MMAC1, FCC and MCC peptides.

Various enzymes also are of interest and may be delivered using the present invention. Such enzymes include but are not limited to cytosine deaminase, adenosine deaminase, hypoxanthine-guanine phosphoribosyltransferase, galactose-1-phosphate uridyltransferase, phenylalanine hydroxylase, glucocerebrosidase, sphingomyelinase, α-L-iduronidase, glucose-6-phosphate dehydrogenase, HSV thymidine kinase and human thymidine kinase.

Another class of proteins that is contemplated to be delivered using the present invention include interleukins (IL) and cytokines. These include but are not limited to interleukin 1 (IL-1), IL-2, IL-3 IL-4, IL-5, IL-6, IL-7, IL-8, IL-9, IL-10, IL-11, IL-12, IL-13, IL-14, IL-15, P-interferon, alpha-interferon, beta-interferon, gamma-interferon, angiostatin, thrombospondin, endostatin, METH-1, METH-2, GM-CSF, G-CSF, M-CSF and tumor necrosis factor.

Cell cycle regulators may also be delivered using the present invention. Such cell cycle regulators include but are not limited to p27, p16, p21, p57, p18, p73, p19, p15, E2F-1, E2F-2, E2F-3, p107, p130 and E2F-4.

In a preferred embodiment, a conjugate of the present invention further comprises a nuclear localization signal. Use of a nuclear localization signal peptide is preferred within a conjugate of the present invention when delivery of compound (d) to the nucleus is desired. Examples of nuclear localization signals of use in the conjugates of the present invention include but are not limited to PKKKRKV of SV40 Large T-antigen (SEQ ID NO: 188) or KRPAATKKAGQAKKKK of nucleoplasmin (SEQ ID NO: 189) [49]. Preferably, a nuclear localization signal is positioned within the conjugate such that if any of the delivery carrier modules (a), (b), or (c) are released from the conjugate via enzymatic or chemical cleavage at a cleavage site within the conjugate, the nuclear localization signal remains linked to compound (d). In another preferred embodiment, a nuclear localization signal is positioned within the conjugate such that if when compound (d) is released from the conjugate via enzymatic or chemical cleavage at a cleavage site within the conjugate, the nuclear localization signal remains linked to compound (d).

In another preferred embodiment, a conjugate of the present invention can be prepared and used to deliver a compound (d) from the ER directly to the nucleus by exploiting the linked membranes of the ER and nucleus (see for example, [50]). Preferably, the conjugate comprises a compound (d) that comprises a DNA molecule, a transcription factor or a small molecule that modulates transcription. In a particularly preferred embodiment, the conjugate comprises at least 2 compounds (d), wherein the first compound (d) is a DNA molecule and the second compound (d) is a transcription factor or a small molecule that modulates transcription.

Preferably, the conjugate comprises, essentially consists of, consists of or contains:

(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
wherein the at least one module (a) is selected from the group consisting of a1, a2, a3, a4, a5, a6, a7, a8, a9, a10, a11, a12, a13, a14, a15, a16, a17, a18, a19, a20, a21, a22, a23, a24, a25, a26, a27, a28, a29, a30, a31, a32, a33, a34, a35, a36, a37, a38, a39, a40, a41, a42, a43, a44, a45, a46, a47, a48, a49, a50, a51, a52, a53, a54, a55, a56, a57, a58, a59, a60, a61, a62, a63, a64, a65, a66, a67, a68, a69, a70, a71, a72, a73, a74, a75, a76, a77, a78, a79, a80, a81, a82, a83, a84, a85, a86, a87, a88, a89, a90, a91, a92, a93, a94, a95, a96, a97, a98, a99, a100, a101, a102, a103, and a104, and wherein the at least one module (a), the at least one module (b), and the at least one module (c), and the at least one compound (d) are linked to each other in any arrangement and stoichiometry.

Preferably, the conjugate comprises, essentially consists of, consists of or contains:

(a) at least one module (a) that mediates cell targeting and facilitates cellular uptake,
b) at least one module (b) that facilitates transport to the ER,
(c) at least one module (c) that mediates translocation from the ER to the cytosol, and
(d) at least one compound (d),
wherein the at least one module (b) is selected from the group consisting of b1, b2, b3, b4, b5, b6, b7, b8, b9, b10, 11b, b12, b13, b14, b15, b16, b17, b18, b19, b20, b21, b22, b23, b24, and b25, and wherein the at least one module (a), the at least one module (b), and the at least one module (c), and the at least one compound (d) are linked to each other in any arrangement and stoichiometry.

Preferably, the conjugate comprises, essentially consists of, consists of or contains:

(a) at least one module (a) that mediates cell targeting and facilitates cellular uptake,
b) at least one module (b) that facilitates transport to the ER,
(c) at least one module (c) that mediates translocation from the ER to the cytosol, and
(d) at least one compound (d),
wherein the at least one module (c) is selected from the group consisting of c1, c2, c3, c4, c5, c6, c7, c8, c9, c10, c11, c12, c13, c14, c15, c16, c17, c18, c19, c20, c21, c22, c23, c24, c25, c26, c27, c28, c29, c30, c31, c32, c33, c34, c35, c36, c37, c38, c39, c40, c41, c42, c43, c44, c45, c46, c47, c48, c49, c50, c51, c52, c53, c54, c55, c56, c57, c58, c59, c60, c61, c62, c63, c64, c65, c66, c67, c68, c69, c70, c71, c72, c73, c74, c75, c76, c77, c78, c79, c80, c81, c82, c83, c84, c85, c86, c87, c88, c89, c90, c91, c92, c93, c94, c95, c96, c97, c98, c99, c100, c101, c102, c103, c104, c105, c106, c107, c108, c109, c110, c111, c112, c113, c114, c115, c116, c117, c118, c119, c120, c121, c122, c123, c124, c125, c126, c127, c128, c129, c130, c131, c132, c133, c134, c135, c136, c137, c138, c139, c140, c141, c142, c143, c144, c145, c146, c147, c148, c149, c150, c151, c152, c153, c154, c155, c156, c157, c158, c159, c160, c161, c162, c163, c164, c165, c166, c167, c168, c169, c170, c171, c172, c173, c174, c175, c176, c177, c178, c179, c180, c181, c182, c183, c184, c185, c186, c187, c188, c189, c190, c191, c192, c193, c194, c195, c196, c197, c198, c199, c200, c201, c202, c203, c204, c205, c206, c207, c208, c209, c210, and c211, and wherein the at least one module (a), the at least one module (b), and the at least one module (c), and the at least one compound (d) are linked to each other in any arrangement and stoichiometry.

Preferably, the conjugate comprises, essentially consists of, consists of or contains:

(a) at least one module (a) that mediates cell targeting and facilitates cellular uptake,
b) at least one module (b) that facilitates transport to the ER,
(c) at least one module (c) that mediates translocation from the ER to the cytosol, and
(d) at least one compound (d),
wherein the at least one compound (d) selected from the group consisting of d1, d2, d3, d4, d5, d6,d7, d8, d9, d10, d11, d12, d13, d14, d15, d16, d17, d18, d19, d20, d21, d22, d23, d24, d25, d26, d27, d28, d29, d30, d31, d32, d33, d34, d35, d36, d37, d38, d39, d40, d41, d42, d43, d44, d45, d46, d47, d48, d49, d50, d51, d52, d53, d54, d55, d56, d57, d58, d59, d60, d61, d62, d63, d64, d65, d66, d67, d68, d69, d70, d71, d72, d73, d74, d75, d76, d77, d78, d79, d80, d81, d82, d83, d84, d85, d86, d87, d88, d89, d90, d91, d92, d93, d94, d95, d96, d97, d98, d99, d100, d101, d102, d103, d104, d105, d106, d107, d108, d109, d110, dill, d112, d113, d114, d115, d116, d117, d118, d119, d120, d121, d122, d123, d124, d125, d126, d127, d128, d129, d130, d131, d132, d133, d134, d135, d136, d137, d138, d139, d140, d141, d142, d143, d144, d145, d146, d147, d148, d149, d150, d151, d152, d153, d154, d155, d156, d157, d158, d159, d160, d161, d162, d163, d164, d165, d166, d167, d168, d169, and d170, and wherein the at least one module (a), the at least one module (b), and the at least one module (c), and the at least one compound (d) are linked to each other in any arrangement and stoichiometry.

Preferably, the conjugate comprises, essentially consists of, consists of or contains:

(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
wherein the at least one module (a) and the at least one module (b) are selected from the group of combinations consisting of a1+b1 (K1), a1+b2 (K2), a1+b3 (K3), a1+b4 (K4), a1+b5 (K5), a1+b6 (K6), a1+b7 (K7), a1+b8 (K8), a1+b9 (K9), a1+b10 (K10), a1+b11 (K11), a1+b12 (K12), a1+b13 (K13), a1+b14 (K14), a1+b15 (K15), a1+b16 (K16), a1+b17 (K17), a1+b18 (K18), a1+b19 (K19), a1+b20 (K20), a1+b21 (K21), a1+b22 (K22), a1+b23 (K23), a1+b24 (K24), a1+b25 (K25), a2+b1 (K26), a2+b2 (K27), a2+b3 (K28), a2+b4 (K29), a2+b5 (K30), a2+b6 (K31), a2+b7 (K32), a2+b8 (K33), a2+b9 (K34), a2+b10 (K35), a2+b11 (K36), a2+b12 (K37), a2+b13 (K38), a2+b14 (K39), a2+b15 (K40), a2+b16 (K41), a2+b17 (K42), a2+b18 (K43), a2+b19 (K44), a2+b20 (K45), a2+b21 (K46), a2+b22 (K47), a2+b23 (K48), a2+b24 (K49), a2+b25 (K50), a3+b1 (K51), a3+b2 (K52), a3+b3 (K53), a3+b4 (K54), a3+b5 (K55), a3+b6 (K56), a3+b7 (K57), a3+b8 (K58), a3+b9 (K59), a3+b10 (K60), a3+b11 (K61), a3+b12 (K62), a3+b13 (K63), a3+b14 (K64), a3+b15 (K65), a3+b16 (K66), a3+b17 (K67), a3+b18 (K68), a3+b19 (K69), a3+b20 (K70), a3+b21 (K71), a3+b22 (K72), a3+b23 (K73), a3+b24 (K74), a3+b25 (K75), a4+b1 (K76), a4+b2 (K77), a4+b3 (K78), a4+b4 (K79), a4+b5 (K80), a4+b6 (K81), a4+b7 (K82), a4+b8 (K83), a4+b9 (K84), a4+b10 (K85), a4+b11 (K86), a4+b12 (K87), a4+b13 (K88), a4+b14 (K89), a4+b15 (K90), a4+b16 (K91), a4+b17 (K92), a4+b18 (K93), a4+b19 (K94), a4+b20 (K95), a4+b21 (K96), a4+b22 (K97), a4+b23 (K98), a4+b24 (K99), a4+b25 (K100), a5+b1 (K101), a5+b2 (K102), a5+b3 (K103), a5+b4 (K104), a5+b5 (K105), a5+b6 (K106), a5+b7 (K107), a5+b8 (K108), a5+b9 (K109), a5+b10 (K110), a5+b11 (K111), a5+b12 (K112), a5+b13 (K113), a5+b14 (K114), a5+b15 (K115), a5+b16 (K116), a5+b17 (K117), a5+b18 (K118), a5+b19 (K119), a5+b20 (K120), a5+b21 (K121), a5+b22 (K122), a5+b23 (K123), a5+b24 (K124), a5+b25 (K125), a6+b1 (K126), a6+b2 (K127), a6+b3 (K128), a6+b4 (K129), a6+b5 (K130), a6+b6 (K131), a6+b7 (K132), a6+b8 (K133), a6+b9 (K134), a6+b10 (K135), a6+b11 (K136), a6+b12 (K137), a6+b13 (K138), a6+b14 (K139), a6+b15 (K140), a6+b16 (K141), a6+b17 (K142), a6+b18 (K143), a6+b19 (K144), a6+b20 (K145), a6+b21 (K146), a6+b22 (K147), a6+b23 (K148), a6+b24 (K149), a6+b25 (K150), a7+b1 (K151), a7+b2 (K152), a7+b3 (K153), a7+b4 (K154), a7+b5 (K155), a7+b6 (K156), a7+b7 (K157), a7+b8 (K158), a7+b9 (K159), a7+b10 (K160), a7+b11 (K161), a7+b12 (K162), a7+b13 (K163), a7+b14 (K164), a7+b15 (K165), a7+b16 (K166), a7+b17 (K167), a7+b18 (K168), a7+b19 (K169), a7+b20 (K170), a7+b21 (K171), a7+b22 (K172), a7+b23 (K173), a7+b24 (K174), a7+b25 (K175), a8+b1 (K176), a8+b2 (K177), a8+b3 (K178), a8+b4 (K179), a8+b5 (K180), a8+b6 (K181), a8+b7 (K182), a8+b8 (K183), a8+b9 (K184), a8+b10 (K185), a8+b11 (K186), a8+b12 (K187), a8+b13 (K188), a8+b14 (K189), a8+b15 (K190), a8+b16 (K191), a8+b17 (K192), a8+b18 (K193), a8+b19 (K194), a8+b20 (K195), a8+b21 (K196), a8+b22 (K197), a8+b23 (K198), a8+b24 (K199), a8+b25 (K200), a9+b1 (K201), a9+b2 (K202), a9+b3 (K203), a9+b4 (K204), a9+b5 (K205), a9+b6 (K206), a9+b7 (K207), a9+b8 (K208), a9+b9 (K209), a9+b10 (K210), a9+b11 (K211), a9+b12 (K212), a9+b13 (K213), a9+b14 (K214), a9+b15 (K215), a9+b16 (K216), a9+b17 (K217), a9+b18 (K218), a9+b19 (K219), a9+b20 (K220), a9+b21 (K221), a9+b22 (K222), a9+b23 (K223), a9+b24 (K224), a9+b25 (K225), a10+b1 (K226), a10+b2 (K227), a10+b3 (K228), a10+b4 (K229), a10+b5 (K230), a10+b6 (K231), a10+b7 (K232), a10+b8 (K233), a10+b9 (K234), a10+b10 (K235), a10+b11 (K236), a10+b12 (K237), a10+b13 (K238), a10+b14 (K239), a10+b15 (K240), a10+b16 (K241), a10+b17 (K242), a10+b18 (K243), a10+b19 (K244), a10+b20 (K245), a10+b21 (K246), a10+b22 (K247), a10+b23 (K248), a10+b24 (K249), a10+b25 (K250), a11+b1 (K251), a11+b2 (K252), a11+b3 (K253), a11+b4 (K254), a11+b5 (K255), a11+b6 (K256), a11+b7 (K257), a11+b8 (K258), a11+b9 (K259), a11+b10 (K260), a11+b11 (K261), a11+b12 (K262), a11+b13 (K263), a11+b14 (K264), a11+b15 (K265), a11+b16 (K266), a11+b17 (K267), a11+b18 (K268), a11+b19 (K269), a11+b20 (K270), a11+b21 (K271), a11+b22 (K272), a11+b23 (K273), a11+b24 (K274), a11+b25 (K275), a12+b1 (K276), a12+b2 (K277), a12+b3 (K278), a12+b4 (K279), a12+b5 (K280), a12+b6 (K281), a12+b7 (K282), a12+b8 (K283), a12+b9 (K284), a12+b10 (K285), a12+b11 (K286), a12+b12 (K287), a12+b13 (K288), a12+b14 (K289), a12+b15 (K290), a12+b16 (K291), a12+b17 (K292), a12+b18 (K293), a12+b19 (K294), a12+b20 (K295), a12+b21 (K296), a12+b22 (K297), a12+b23 (K298), a12+b24 (K299), a12+b25 (K300), a13+b1 (K301), a13+b2 (K302), a13+b3 (K303), a13+b4 (K304), a13+b5 (K305), a13+b6 (K306), a13+b7 (K307), a13+b8 (K308), a13+b9 (K309), a13+b10 (K310), a13+b11 (K311), a13+b12 (K312), a13+b13 (K313), a13+b14 (K314), a13+b15 (K315), a13+b16 (K316), a13+b17 (K317), a13+b18 (K318), a13+b19 (K319), a13+b20 (K320), a13+b21 (K321), a13+b22 (K322), a13+b23 (K323), a13+b24 (K324), a13+b25 (K325), a14+b1 (K326), a14+b2 (K327), a14+b3 (K328), a14+b4 (K2329), a14+b5 (K330), a14+b6 (K331), a14+b7 (K332), a14+b8 (K333), a14+b9 (K334), a14+b10 (K335), a14+b11 (K336), a14+b12 (K337), a14+b13 (K338), a14+b14 (K339), a14+b15 (K340), a14+b16 (K341), a14+b17 (K342), a14+b18 (K343), a14+b19 (K344), a14+b20 (K345), a14+b21 (K346), a14+b22 (K347), a14+b23 (K348), a14+b24 (K349), a14+b25 (K350), a15+b1 (K351), a15+b2 (K352), a15+b3 (K353), a15+b4 (K354), a15+b5 (K355), a15+b6 (K356), a15+b7 (K357), a15+b8 (K358), a15+b9 (K359), a15+b10 (K360), a15+b11 (K361), a15+b12 (K362), a15+b13 (K363), a15+b14 (K364), a15+b15 (K365), a15+b16 (K366), a15+b17 (K367), a15+b18 (K368), a15+b19 (K369), a15+b20 (K370), a15+b21 (K371), a15+b22 (K372), a15+b23 (K373), a15+b24 (K374), a15+b25 (K375), a16+b1 (K376), a16+b2 (K377), a16+b3 (K378), a16+b4 (K379), a16+b5 (K380), a16+b6 (K381), a16+b7 (K382), a16+b8 (K383), a16+b9 (K384), a16+b10 (K385), a16+b11 (K386), a16+b12 (K387), a16+b13 (K388), a16+b14 (K389), a16+b15 (K390), a16+b16 (K391), a16+b17 (K392), a16+b18 (K393), a16+b19 (K394), a16+b20 (K395), a16+b21 (K396), a16+b22 (K397), a16+b23 (K398), a16+b24 (K399), a16+b25 (K400), a17+b1 (K401), a17+b2 (K402), a17+b3 (K403), a17+b4 (K404), a17+b5 (K405), a17+b6 (K406), a17+b7 (K407), a17+b8 (K408), a17+b9 (K409), a17+b10 (K410), a17+b11 (K411), a17+b12 (K412), a17+b13 (K413), a17+b14 (K414), a17+b15 (K415), a17+b16 (K416), a17+b17 (K417), a17+b18 (K418), a17+b19 (K419), a17+b20 (K420), a17+b21 (K421), a17+b22 (K422), a17+b23 (K423), a17+b24 (K424), a17+b25 (K425), a18+b1 (K426), a18+b2 (K427), a18+b3 (K428), a18+b4 (K429), a18+b5 (K430), a18+b6 (K431), a18+b7 (K432), a18+b8 (K433), a18+b9 (K434), a18+b10 (K435), a18+b11 (K436), a18+b12 (K437), a18+b13 (K438), a18+b14 (K439), a18+b15 (K440), a18+b16 (K441), a18+b17 (K442), a18+b18 (K443), a18+b19 (K444), a18+b20 (K445), a18+b21 (K446), a18+b22 (K447), a18+b23 (K448), a18+b24 (K449), a18+b25 (K450), a19+b1 (K451), a19+b2 (K452), a19+b3 (K453), a19+b4 (K454), a19+b5 (K455), a19+b6 (K456), a19+b7 (K457), a19+b8 (K458), a19+b9 (K459), a19+b10 (K460), a19+b11 (K461), a19+b12 (K462), a19+b13 (K463), a19+b14 (K464), a19+b15 (K465), a19+b16 (K466), a19+b17 (K467), a19+b18 (K468), a19+b19 (K469), a19+b20 (K470), a19+b21 (K471), a19+b22 (K472), a19+b23 (K473), a19+b24 (K474), a19+b25 (K475), a20+b1 (K476), a20+b2 (K477), a20+b3 (K478), a20+b4 (K479), a20+b5 (K480), a20+b6 (K481), a20+b7 (K482), a20+b8 (K483), a20+b9 (K484), a20+b10 (K485), a20+b11 (K486), a20+b12 (K487), a20+b13 (K488), a20+b14 (K489), a20+b15 (K490), a20+b16 (K491), a20+b17 (K492), a20+b18 (K493), a20+b19 (K494), a20+b20 (K495), a20+b21 (K496), a20+b22 (K497), a20+b23 (K498), a20+b24 (K499), a20+b25 (K500), a21+b1 (K501), a21+b2 (K502), a21+b3 (K503), a21+b4 (K504), a21+b5 (K505), a21+b6 (K506), a21+b7 (K507), a21+b8 (K508), a21+b9 (K509), a21+b10 (K510), a21+b11 (K511), a21+b12 (K512), a21+b13 (K513), a21+b14 (K514), a21+b15 (K515), a21+b16 (K516), a21+b17 (K517), a21+b18 (K518), a21+b19 (K519), a21+b20 (K520), a21+b21 (K521), a21+b22 (K522), a21+b23 (K523), a21+b24 (K524), a21+b25 (K525), a22+b1 (K526), a22+b2 (K527), a22+b3 (K528), a22+b4 (K529), a22+b5 (K530), a22+b6 (K531), a22+b7 (K532), a22+b8 (K533), a22+b9 (K534), a22+b10 (K535), a22+b11 (K536), a22+b12 (K537), a22+b13 (K538), a22+b14 (K539), a22+b15 (K540), a22+b16 (K541), a22+b17 (K542), a22+b18 (K543), a22+b19 (K544), a22+b20 (K545), a22+b21 (K546), a22+b22 (K547), a22+b23 (K548), a22+b24 (K549), a22+b25 (K550), a23+b1 (K551), a23+b2 (K552), a23+b3 (K553), a23+b4 (K554), a23+b5 (K555), a23+b6 (K556), a23+b7 (K557), a23+b8 (K558), a23+b9 (K559), a23+b10 (K560), a23+b11 (K561), a23+b12 (K562), a23+b13 (K563), a23+b14 (K564), a23+b15 (K565), a23+b16 (K566), a23+b17 (K567), a23+b18 (K568), a23+b19 (K569), a23+b20 (K570), a23+b21 (K571), a23+b22 (K572), a23+b23 (K573), a23+b24 (K574), a23+b25 (K575), a24+b1 (K576), a24+b2 (K577), a24+b3 (K578), a24+b4 (K579), a24+b5 (K580), a24+b6 (K581), a24+b7 (K582), a24+b8 (K583), a24+b9 (K584), a24+b10 (K585), a24+b11 (K586), a24+b12 (K587), a24+b13 (K588), a24+b14 (K589), a24+b15 (K590), a24+b16 (K591), a24+b17 (K592), a24+b18 (K593), a24+b19 (K594), a24+b20 (K595), a24+b21 (K596), a24+b22 (K597), a24+b23 (K598), a24+b24 (K599), a24+b25 (K600), a25+b1 (K601), a25+b2 (K602), a25+b3 (K603), a25+b4 (K604), a25+b5 (K605), a25+b6 (K606), a25+b7 (K607), a25+b8 (K608), a25+b9 (K609), a25+b10 (K610), a25+b11 (K611), a25+b12 (K612), a25+b13 (K613), a25+b14 (K614), a25+b15 (K615), a25+b16 (K616), a25+b17 (K617), a25+b18 (K618), a25+b19 (K619), a25+b20 (K620), a25+b21 (K621), a25+b22 (K622), a25+b23 (K623), a25+b24 (K624), a25+b25 (K625), a26+b1 (K626), a26+b2 (K627), a26+b3 (K628), a26+b4 (K629), a26+b5 (K630), a26+b6 (K631), a26+b7 (K632), a26+b8 (K633), a26+b9 (K634), a26+b10 (K635), a26+b11 (K636), a26+b12 (K637), a26+b13 (K638), a26+b14 (K639), a26+b15 (K640), a26+b16 (K641), a26+b17 (K642), a26+b18 (K643), a26+b19 (K644), a26+b20 (K645), a26+b21 (K646), a26+b22 (K647), a26+b23 (K648), a26+b24 (K649), a26+b25 (K650), a27+b1 (K651), a27+b2 (K652), a27+b3 (K653), a27+b4 (K654), a27+b5 (K655), a27+b6 (K656), a27+b7 (K657), a27+b8 (K658), a27+b9 (K659), a27+b10 (K660), a27+b11 (K661), a27+b12 (K662), a27+b13 (K663), a27+b14 (K664), a27+b15 (K665), a27+b16 (K666), a27+b17 (K667), a27+b18 (K668), a27+b19 (K669), a27+b20 (K670), a27+b21 (K671), a27+b22 (K672), a27+b23 (K673), a27+b24 (K674), a27+b25 (K675), a28+b1 (K676), a28+b2 (K677), a28+b3 (K678), a28+b4 (K679), a28+b5 (K680), a28+b6 (K681), a28+b7 (K682), a28+b8 (K683), a28+b9 (K684), a28+b10 (K685), a28+b11 (K686), a28+b12 (K687), a28+b13 (K688), a28+b14 (K689), a28+b15 (K690), a28+b16 (K691), a28+b17 (K692), a28+b18 (K693), a28+b19 (K694), a28+b20 (K695), a28+b21 (K696), a28+b22 (K697), a28+b23 (K698), a28+b24 (K699), a28+b25 (K700), a29+b1 (K701), a29+b2 (K702), a29+b3 (K703), a29+b4 (K704), a29+b5 (K705), a29+b6 (K706), a29+b7 (K707), a29+b8 (K708), a29+b9 (K709), a29+b10 (K710), a29+b11 (K711), a29+b12 (K712), a29+b13 (K713), a29+b14 (K714), a29+b15 (K715), a29+b16 (K716), a29+b17 (K717), a29+b18 (K718), a29+b19 (K719), a29+b20 (K720), a29+b21 (K721), a29+b22 (K722), a29+b23 (K723), a29+b24 (K724), a29+b25 (K725), a30+b1 (K726), a30+b2 (K727), a30+b3 (K728), a30+b4 (K729), a30+b5 (K730), a30+b6 (K731), a30+b7 (K732), a30+b8 (K733), a30+b9 (K734), a30+b10 (K735), a30+b11 (K736), a30+b12 (K737), a30+b13 (K738), a30+b14 (K739), a30+b15 (K740), a30+b16 (K741), a30+b17 (K742), a30+b18 (K743), a30+b19 (K744), a30+b20 (K745), a30+b21 (K746), a30+b22 (K747), a30+b23 (K748), a30+b24 (K749), a30+b25 (K750), a31+b1 (K751), a31+b2 (K752), a31+b3 (K753), a31+b4 (K754), a31+b5 (K755), a31+b6 (K756), a31+b7 (K757), a31+b8 (K758), a31+b9 (K759), a31+b10 (K760), a31+b11 (K761), a31+b12 (K762), a31+b13 (K763), a31+b14 (K764), a31+b15 (K765), a31+b16 (K766), a31+b17 (K767), a31+b18 (K768), a31+b19 (K769), a31+b20 (K770), a31+b21 (K771), a31+b22 (K772), a31+b23 (K773), a31+b24 (K774), a31+b25 (K775), a32+b1 (776), a32+b2 (K777), a32+b3 (K778), a32+b4 (K779), a32+b5 (K780), a32+b6 (K781), a32+b7 (K782), a32+b8 (K783), a32+b9 (K784), a32+b10 (K785), a32+b11 (K786), a32+b12 (K787), a32+b13 (K788), a32+b14 (K789), a32+b15 (K790), a32+b16 (K791), a32+b17 (K792), a32+b18 (K793), a32+b19 (K794), a32+b20 (K795), a32+b21 (K796), a32+b22 (K797), a32+b23 (K798), a32+b24 (K799), a32+b25 (K800), a33+b1 (K801), a33+b2 (K802), a33+b3 (K803), a33+b4 (K804), a33+b5 (K805), a33+b6 (K806), a33+b7 (K807), a33+b8 (K808), a33+b9 (K809), a33+b10 (K810), a33+b11 (K811), a33+b12 (K812), a33+b13 (K813), a33+b14 (K814), a33+b15 (K815), a33+b16 (K816), a33+b17 (K817), a33+b18 (K818), a33+b19 (K819), a33+b20 (K820), a33+b21 (K821), a33+b22 (K822), a33+b23 (K823), a33+b24 (K824), a33+b25 (K825), a34+b1 (K826), a34+b2 (K827), a34+b3 (K828), a34+b4 (K829), a34+b5 (K830), a34+b6 (K831), a34+b7 (K832), a34+b8 (K833), a34+b9 (K834), a34+b10 (K835), a34+b11 (K836), a34+b12 (K837), a34+b13 (K838), a34+b14 (K839), a34+b15 (K840), a34+b16 (K841), a34+b17 (K842), a34+b18 (K843), a34+b19 (K844), a34+b20 (K845), a34+b21 (K846), a34+b22 (K847), a34+b23 (K848), a34+b24 (K849), a34+b25 (K850), a35+b1 (K851), a35+b2 (K852), a35+b3 (K853), a35+b4 (K854), a35+b5 (K855), a35+b6 (K856), a35+b7 (K857), a35+b8 (K858), a35+b9 (K859), a35+b10 (K860), a35+b11 (K861), a35+b12 (K862), a35+b13 (K863), a35+b14 (K864), a35+b15 (K865), a35+b16 (K866), a35+b17 (K867), a35+b18 (K868), a35+b19 (K869), a35+b20 (K870), a35+b21 (K871), a35+b22 (K872), a35+b23 (K873), a35+b24 (K874), a35+b25 (K875), a36+b1 (K876), a36+b2 (K877), a36+b3 (K878), a36+b4 (K879), a36+b5 (K880), a36+b6 (K881), a36+b7 (K882), a36+b8 (K883), a36+b9 (K884), a36+b10 (K885), a36+b11 (K886), a36+b12 (K887), a36+b13 (K888), a36+b14 (K889), a36+b15 (K890), a36+b16 (K891), a36+b17 (K892), a36+b18 (K893), a36+b19 (K894), a36+b20 (K895), a36+b21 (K896), a36+b22 (K897), a36+b23 (K898), a36+b24 (K899), a36+b25 (K900), a37+b1 (K901), a37+b2 (K902), a37+b3 (K903), a37+b4 (K904), a37+b5 (K905), a37+b6 (K906), a37+b7 (K907), a37+b8 (K908), a37+b9 (K909), a37+b10 (K910), a37+b11 (K911), a37+b12 (K912), a37+b13 (K913), a37+b14 (K914), a37+b15 (K915), a37+b16 (K916), a37+b17 (K917), a37+b18 (K918), a37+b19 (K919), a37+b20 (K920), a37+b21 (K921), a37+b22 (K922), a37+b23 (K923), a37+b24 (K924), a37+b25 (K925), a38+b1 (K926), a38+b2 (K927), a38+b3 (K928), a38+b4 (K929), a38+b5 (K930), a38+b6 (K931), a38+b7 (K932), a38+b8 (K933), a38+b9 (K934), a38+b10 (K935), a38+b11 (K936), a38+b12 (K937), a38+b13 (K938), a38+b14 (K939), a38+b15 (K940), a38+b16 (K941), a38+b17 (K942), a38+b18 (K943), a38+b19 (K944), a38+b20 (K945), a38+b21 (K946), a38+b22 (K947), a38+b23 (K948), a38+b24 (K949), a38+b25 (K950), a39+b1 (K951), a39+b2 (K952), a39+b3 (K953), a39+b4 (K954), a39+b5 (K955), a39+b6 (K956), a39+b7 (K957), a39+b8 (K958), a39+b9 (K959), a39+b10 (K960), a39+b11 (K961), a39+b12 (K962), a39+b13 (K963), a39+b14 (K964), a39+b15 (K965), a39+b16 (K966), a39+b17 (K967), a39+b18 (K968), a39+b19 (K969), a39+b20 (K970), a39+b21 (K971), a39+b22 (K972), a39+b23 (K973), a39+b24 (K974), a39+b25 (K975), a40+b1 (K976), a40+b2 (K977), a40+b3 (K978), a40+b4 (K979), a40+b5 (K980), a40+b6 (K981), a40+b7 (K982), a40+b8 (K983), a40+b9 (K984), a40+b10 (K985), a40+b11 (K986), a40+b12 (K987), a40+b13 (K988), a40+b14 (K989), a40+b15 (K990), a40+b16 (K991), a40+b17 (K992), a40+b18 (K993), a40+b19 (K994), a40+b20 (K995), a40+b21 (K996), a40+b22 (K997), a40+b23 (K998), a40+b24 (K999), a40+b25 (K1000), a41+b1 (K1001), a41+b2 (K1002), a41+b3 (K1003), a41+b4 (K1004), a41+b5 (K1005), a41+b6 (K1006), a41+b7 (K1007), a41+b8 (K1008), a41+b9 (K1009), a41+b10 (K1010), a41+b11 (K1011), a41+b12 (K1012), a41+b13 (K1013), a41+b14 (K1014), a41+b15 (K1015), a41+b16 (K1016), a41+b17 (K1017), a41+b18 (K1018), a41+b19 (K1019), a41+b20 (K1020), a41+b21 (K1021), a41+b22 (K1022), a41+b23 (K1023), a41+b24 (K1024), a41+b25 (K1025), a42+b1 (K1026), a42+b2 (K1027), a42+b3 (K1028), a42+b4 (K1029), a42+b5 (K1030), a42+b6 (K1031), a42+b7 (K1032), a42+b8 (K1033), a42+b9 (K1034), a42+b10 (K1035), a42+b11 (K1036), a42+b12 (K1037), a42+b13 (K1038), a42+b14 (K1039), a42+b15 (K1040), a42+b16 (K1041), a42+b17 (K1042), a42+b18 (K1043), a42+b19 (K1044), a42+b20 (K1045), a42+b21 (K1046), a42+b22 (K1047), a42+b23 (K1048), a42+b24 (K1049), a42+b25 (K1050), a43+b1 (K1051), a43+b2 (K1052), a43+b3 (K1053), a43+b4 (K1054), a43+b5 (K1055), a43+b6 (K1056), a43+b7 (K1057), a43+b8 (K1058), a43+b9 (K1059), a43+b10 (K1060), a43+b11 (K1061), a43+b12 (K1062), a43+b13 (K1063), a43+b14 (K1064), a43+b15 (K1065), a43+b16 (K1066), a43+b17 (K1067), a43+b18 (K1068), a43+b19 (K1069), a43+b20 (K1070), a43+b21 (K1071), a43+b22 (K1072), a43+b23 (K1073), a43+b24 (K1074), a43+b25 (K1075), a44+b1 (K1076), a44+b2 (K1077), a44+b3 (K1078), a44+b4 (K1079), a44+b5 (K1080), a44+b6 (K1081), a44+b7 (K1082), a44+b8 (K1083), a44+b9 (K1084), a44+b10 (K1085), a44+b11 (K1086), a44+b12 (K1087), a44+b13 (K1088), a44+b14 (K1089), a44+b15 (K1090), a44+b16 (K1091), a44+b17 (K1092), a44+b18 (K1093), a44+b19 (K1094), a44+b20 (K1095), a44+b21 (K1096), a44+b22 (K1097), a44+b23 (K1098), a44+b24 (K1099), a44+b25 (K1100), a45+b1 (K1101), a45+b2 (K1102), a45+b3 (K1103), a45+b4 (K1104), a45+b5 (K1105), a45+b6 (K1106), a45+b7 (K1107), a45+b8 (K1108), a45+b9 (K1109), a45+b10 (K1110), a45+b11 (K1111), a45+b12 (K1112), a45+b13 (K1113), a45+b14 (K1114), a45+b15 (K1115), a45+b16 (K1116), a45+b17 (K1117), a45+b18 (K1118), a45+b19 (K1119), a45+b20 (K1120), a45+b21 (K1121), a45+b22 (K1122), a45+b23 (K1123), a45+b24 (K1124), a45+b25 (K1125), a46+b1 (K1126), a46+b2 (K1127), a46+b3 (K1128), a46+b4 (K1129), a46+b5 (K1130), a46+b6 (K1131), a46+b7 (K1132), a46+b8 (K1133), a46+b9 (K1134), a46+b10 (K1135), a46+b11 (K1136), a46+b12 (K1137), a46+b13 (K1138), a46+b14 (K1139), a46+b15 (K1140), a46+b16 (K1141), a46+b17 (K1142), a46+b18 (K1143), a46+b19 (K1144), a46+b20 (K1145), a46+b21 (K1146), a46+b22 (K1147), a46+b23 (K1148), a46+b24 (K1149), a46+b25 (K1150), a47+b1 (K1151), a47+b2 (K1152), a47+b3 (K1153), a47+b4 (K1154), a47+b5 (K1155), a47+b6 (K1156), a47+b7 (K1157), a47+b8 (K1158), a47+b9 (K1159), a47+b10 (K1160), a47+b11 (K1161), a47+b12 (K1162), a47+b13 (K1163), a47+b14 (K1164), a47+b15 (K1165), a47+b16 (K1166), a47+b17 (K1167), a47+b18 (K1168), a47+b19 (K1169), a47+b20 (K1170), a47+b21 (K1171), a47+b22 (K1172), a47+b23 (K1173), a47+b24 (K1174), a47+b25 (K1175), a48+b1 (K1176), a48+b2 (K1177), a48+b3 (K1178), a48+b4 (1179), a48+b5 (K1180), a48+b6 (K1181), a48+b7 (K1182), a48+b8 (K1183), a48+b9 (K1184), a48+b10 (K1185), a48+b11 (K1186), a48+b12 (K1187), a48+b13 (K1188), a48+b14 (K1189), a48+b15 (K1190), a48+b16 (K1191), a48+b17 (K1192), a48+b18 (K1193), a48+b19 (K1194), a48+b20 (K1195), a48+b21 (K1196), a48+b22 (K1197), a48+b23 (K1198), a48+b24 (K1199), a48+b25 (K1200), a49+b1 (K1201), a49+b2 (K1202), a49+b3 (K1203), a49+b4 (K1204), a49+b5 (K1205), a49+b6 (K1206), a49+b7 (K1207), a49+b8 (K1208), a49+b9 (K1209), a49+b10 (K1210), a49+b11 (K1211), a49+b12 (K1212), a49+b13 (K1213), a49+b14 (K1214), a49+b15 (K1215), a49+b16 (K1216), a49+b17 (K1217), a49+b18 (K1218), a49+b19 (K1219), a49+b20 (K1220), a49+b21 (K1221), a49+b22 (K1222), a49+b23 (K1223), a49+b24 (K1224), a49+b25 (K1225), a50+b1 (K1226), a50+b2 (K1227), a50+b3 (K1228), a50+b4 (K1229), a50+b5 (K1230), a50+b6 (K1231), a50+b7 (K1232), a50+b8 (K1233), a50+b9 (K1234), a50+b10 (K1235), a50+b11 (K1236), a50+b12 (K1237), a50+b13 (K1238), a50+b14 (K1239), a50+b15 (K1240), a50+b16 (K1241), a50+b17 (K1242), a50+b18 (K1243), a50+b19 (K1244), a50+b20 (K1245), a50+b21 (K1246), a50+b22 (K1247), a50+b23 (K1248), a50+b24 (K1249), a50+b25 (K1250), a51+b1 (K1251), a51+b2 (K1252), a51+b3 (K1253), a51+b4 (K1254), a51+b5 (K1255), a51+b6 (K1256), a51+b7 (K1257), a51+b8 (K1258), a51+b9 (K1259), a51+b10 (K1260), a51+b11 (K1261), a51+b12 (K1262), a51+b13 (K1263), a51+b14 (K1264), a51+b15 (K1265), a51+b16 (K1266), a51+b17 (K1267), a51+b18 (K1268), a51+b19 (K1269), a51+b20 (K1270), a51+b21 (K1271), a51+b22 (K1272), a51+b23 (K1273), a51+b24 (K1274), a51+b25 (K1275), a52+b1 (K1276), a52+b2 (K1277), a52+b3 (K1278), a52+b4 (K1279), a52+b5 (K1280), a52+b6 (K1281), a52+b7 (K1282), a52+b8 (K1283), a52+b9 (K1284), a52+b10 (K1285), a52+b11 (K1286), a52+b12 (K1287), a52+b13 (K1288), a52+b14 (K1289), a52+b15 (K1290), a52+b16 (K1291), a52+b17 (K1292), a52+b18 (K1293), a52+b19 (K1294), a52+b20 (K1295), a52+b21 (K1296), a52+b22 (K1297), a52+b23 (K1298), a52+b24 (K1299), a52+b25 (K1300), a53+b1 (K1301), a53+b2 (K1302), a53+b3 (K1303), a53+b4 (K1304), a53+b5 (K1305), a53+b6 (K1306), a53+b7 (K1307), a53+b8 (K1308), a53+b9 (K1309), a53+b10 (K1310), a53+b11 (K1311), a53+b12 (K1312), a53+b13 (K1313), a53+b14 (K1314), a53+b15 (K1315), a53+b16 (K1316), a53+b17 (K1317), a53+b18 (K1318), a53+b19 (K1319), a53+b20 (K1320), a53+b21 (K1321), a53+b22 (K1322), a53+b23 (K1323), a53+b24 (K1324), a53+b25 (K1325), a54+b1 (K1326), a54+b2 (K1327), a54+b3 (K1328), a54+b4 (K1329), a54+b5 (K1330), a54+b6 (K1331), a54+b7 (K1332), a54+b8 (K1333), a54+b9 (K1334), a54+b10 (K1335), a54+b11 (K1336), a54+b12 (K1337), a54+b13 (K1338), a54+b14 (K1339), a54+b15 (K1340), a54+b16 (K1341), a54+b17 (K1342), a54+b18 (K1343), a54+b19 (K1344), a54+b20 (K1345), a54+b21 (K1346), a54+b22 (K1347), a54+b23 (K1348), a54+b24 (K1349), a54+b25 (K1350), a55+b1 (K1351), a55+b2 (K1352), a55+b3 (K1353), a55+b4 (K1354), a55+b5 (K1355), a55+b6 (K1356), a55+b7 (K1357), a55+b8 (K1358), a55+b9 (K1359), a55+b10 (K1360), a55+b11 (K1361), a55+b12 (K1362), a55+b13 (K1363), a55+b14 (K1364), a55+b15 (K1365), a55+b16 (K1366), a55+b17 (K1367), a55+b18 (K1368), a55+b19 (K1369), a55+b20 (K1370), a55+b21 (K1371), a55+b22 (K1372), a55+b23 (K1373), a55+b24 (K1374), a55+b25 (K1375), a56+b1 (K1376), a56+b2 (K1377), a56+b3 (K1378), a56+b4 (K1379), a56+b5 (K1380), a56+b6 (K1381), a56+b7 (K1382), a56+b8 (K1383), a56+b9 (K1384), a56+b10 (K1385), a56+b11 (K1386), a56+b12 (K1387), a56+b13 (K1388), a56+b14 (K1389), a56+b15 (K1390), a56+b16 (K1391), a56+b17 (K1392), a56+b18 (K1393), a56+b19 (K1394), a56+b20 (K1395), a56+b21 (K1396), a56+b22 (K1397), a56+b23 (K1398), a56+b24 (K1399), a56+b25 (K1400), a57+b1 (K1401), a57+b2 (K1402), a57+b3 (K1403), a57+b4 (K1404), a57+b5 (K1405), a57+b6 (K1406), a57+b7 (K1407), a57+b8 (K1408), a57+b9 (K1409), a57+b10 (K1410), a57+b11 (K1411), a57+b12 (K1412), a57+b13 (K1413), a57+b14 (K1414), a57+b15 (K1415), a57+b16 (K1416), a57+b17 (K1417), a57+b18 (K1418), a57+b19 (K1419), a57+b20 (K1420), a57+b21 (K1421), a57+b22 (K1422), a57+b23 (K1423), a57+b24 (K1424), a57+b25 (K1425), a58+b1 (K1426), a58+b2 (K1427), a58+b3 (K1428), a58+b4 (K1429), a58+b5 (K1430), a58+b6 (K1431), a58+b7 (K1432), a58+b8 (K1433), a58+b9 (K1434), a58+b10 (K1435), a58+b11 (K1436), a58+b12 (K1437), a58+b13 (K1438), a58+b14 (K1439), a58+b15 (K1440), a58+b16 (K1441), a58+b17 (K1442), a58+b18 (K1443), a58+b19 (K1444), a58+b20 (K1445), a58+b21 (K1446), a58+b22 (K1447), a58+b23 (K1448), a58+b24 (K1449), a58+b25 (K1450), a59+b1 (K1451), a59+b2 (K1452), a59+b3 (K1453), a59+b4 (K1454), a59+b5 (K1455), a59+b6 (K1456), a59+b7 (K1457), a59+b8 (K1458), a59+b9 (K1459), a59+b10 (K1460), a59+b11 (K1461), a59+b12 (K1462), a59+b13 (K1463), a59+b14 (K1464), a59+b15 (K1465), a59+b16 (K1466), a59+b17 (K1467), a59+b18 (K1468), a59+b19 (K1469), a59+b20 (K1470), a59+b21 (K1471), a59+b22 (K1472), a59+b23 (K1473), a59+b24 (K1474), a59+b25 (K1475), a60+b1 (K1476), a60+b2 (K1477), a60+b3 (K1478), a60+b4 (K1479), a60+b5 (K1480), a60+b6 (K1481), a60+b7 (K1482), a60+b8 (K1483), a60+b9 (K1484), a60+b10 (K1485), a60+b11 (K1486), a60+b12 (K1487), a60+b13 (K1488), a60+b14 (K1489), a60+b15 (K1490), a60+b16 (K1491), a60+b17 (K1492), a60+b18 (K1493), a60+b19 (K1494), a60+b20 (K1495), a60+b21 (K1496), a60+b22 (K1497), a60+b23 (K1498), a60+b24 (K1499), a60+b25 (K1500), a61+b1 (K1501), a61+b2 (K1502), a61+b3 (K1503), a61+b4 (K1504), a61+b5 (K1505), a61+b6 (K1506), a61+b7 (K1507), a61+b8 (K1508), a61+b9 (K1509), a61+b10 (K1510), a61+b11 (K1511), a61+b12 (K1512), a61+b13 (K1513), a61+b14 (K1514), a61+b15 (K1515), a61+b16 (K1516), a61+b17 (K1517), a61+b18 (K1518), a61+b19 (K1519), a61+b20 (K1520), a61+b21 (K1521), a61+b22 (K1522), a61+b23 (K1523), a61+b24 (K1524), a61+b25 (K1525), a62+b1 (K1526), a62+b2 (K1527), a62+b3 (K1528), a62+b4 (K1529), a62+b5 (K1530), a62+b6 (K1531), a62+b7 (K1532), a62+b8 (K1533), a62+b9 (K1534), a62+b10 (K1535), a62+b11 (K1536), a62+b12 (K1537), a62+b13 (K1538), a62+b14 (K1539), a62+b15 (K1540), a62+b16 (K1541), a62+b17 (K1542), a62+b18 (K1543), a62+b19 (K1544), a62+b20 (K1545), a62+b21 (K1546), a62+b22 (K1547), a62+b23 (K1548), a62+b24 (K1549), a62+b25 (K1550), a63+b1 (K1551), a63+b2 (K1552), a63+b3 (K1553), a63+b4 (K1554), a63+b5 (K1555), a63+b6 (K1556), a63+b7 (K1557), a63+b8 (K1558), a63+b9 (K1559), a63+b10 (K1560), a63+b11 (K1561), a63+b12 (K1562), a63+b13 (K1563), a63+b14 (K1564), a63+b15 (K1565), a63+b16 (K1566), a63+b17 (K1567), a63+b18 (K1568), a63+b19 (K1569), a63+b20 (K1570), a63+b21 (K1571), a63+b22 (K1572), a63+b23 (K1573), a63+b24 (K1574), a63+b25 (K1575), a64+b1 (K1576), a64+b2 (K1577), a64+b3 (K1578), a64+b4 (K1579), a64+b5 (K1580), a64+b6 (K1581), a64+b7 (K1582), a64+b8 (K1583), a64+b9 (K1584), a64+b10 (K1585), a64+b11 (K1586), a64+b12 (K1587), a64+b13 (K1588), a64+b14 (K1589), a64+b15 (K1590), a64+b16 (K1591), a64+b17 (K1592), a64+b18 (K1593), a64+b19 (K1594), a64+b20 (K1595), a64+b21 (K1596), a64+b22 (K1597), a64+b23 (K1598), a64+b24 (K1599), a64+b25 (K1600), a65+b1 (K1601), a65+b2 (K1602), a65+b3 (K1603), a65+b4 (K1604), a65+b5 (K1605), a65+b6 (K1606), a65+b7 (K1607), a65+b8 (K1608), a65+b9 (K1609), a65+b10 (K1610), a65+b11 (K1611), a65+b12 (K1612), a65+b13 (K1613), a65+b14 (K1614), a65+b15 (K1615), a65+b16 (K1616), a65+b17 (K1617), a65+b18 (K1618), a65+b19 (K1619), a65+b20 (K1620), a65+b21 (K1621), a65+b22 (K1622), a65+b23 (K1623), a65+b24 (K1624), a65+b25 (K1625), a66+b1 (K1626), a66+b2 (K1627), a66+b3 (K1628), a66+b4 (K1629), a66+b5 (K1630), a66+b6 (K1631), a66+b7 (K1632), a66+b8 (K1633), a66+b9 (K1634), a66+b10 (K1635), a66+b11 (K1636), a66+b12 (K1637), a66+b13 (K1638), a66+b14 (K1639), a66+b15 (K1640), a66+b16 (K1641), a66+b17 (K1642), a66+b18 (K1643), a66+b19 (K1644), a66+b20 (K1645), a66+b21 (K1646), a66+b22 (K1647), a66+b23 (K1648), a66+b24 (K1649), a66+b25 (K1650), a67+b1 (K1651), a67+b2 (K1652), a67+b3 (K1653), a67+b4 (K1654), a67+b5 (K1655), a67+b6 (K1656), a67+b7 (K1657), a67+b8 (K1658), a67+b9 (K1659), a67+b10 (K1660), a67+b11 (K1661), a67+b12 (K1662), a67+b13 (K1663), a67+b14 (K1664), a67+b15 (K1665), a67+b16 (K1666), a67+b17 (K1667), a67+b18 (K1668), a67+b19 (K1669), a67+b20 (K1670), a67+b21 (K1671), a67+b22 (K1672), a67+b23 (K1673), a67+b24 (K1674), a67+b25 (K1675), a68+b1 (K1676), a68+b2 (K1677), a68+b3 (K1678), a68+b4 (K1679), a68+b5 (K1680), a68+b6 (K1681), a68+b7 (K1682), a68+b8 (K1683), a68+b9 (K1684), a68+b10 (K1685), a68+b11 (K1686), a68+b12 (K1687), a68+b13 (K1688), a68+b14 (K1689), a68+b15 (K1690), a68+b16 (K1691), a68+b17 (K1692), a68+b18 (K1693), a68+b19 (K1694), a68+b20 (K1695), a68+b21 (K1696), a68+b22 (K1697), a68+b23 (K1698), a68+b24 (K1699), a68+b25 (K1700), a69+b1 (K1701), a69+b2 (K1702), a69+b3 (K1703), a69+b4 (K1704), a69+b5 (K1705), a69+b6 (K1706), a69+b7 (K1707), a69+b8 (K1708), a69+b9 (K1709), a69+b10 (K1710), a69+b11 (K1711), a69+b12 (K1712), a69+b13 (K1713), a69+b14 (K1714), a69+b15 (K1715), a69+b16 (K1716), a69+b17 (K1717), a69+b18 (K1718), a69+b19 (K1719), a69+b20 (K1720), a69+b21 (K1721), a69+b22 (K1722), a69+b23 (K1723), a69+b24 (K1724), a69+b25 (K1725), a70+b1 (K1726), a70+b2 (K1727), a70+b3 (K1728), a70+b4 (K1729), a70+b5 (K1730), a70+b6 (K1731), a70+b7 (K1732), a70+b8 (K1733), a70+b9 (K1734), a70+b10 (K1735), a70+b11 (K1736), a70+b12 (K1737), a70+b13 (K1738), a70+b14 (K1739), a70+b15 (K1740), a70+b16 (K1741), a70+b17 (K1742), a70+b18 (K1743), a70+b19 (K1744), a70+b20 (K1745), a70+b21 (K1746), a70+b22 (K1747), a70+b23 (K1748), a70+b24 (K1749), a70+b25 (K1750), a71+b1 (K4919), a71+b2 (K4920), a71+b3 (K4921), a71+b4 (K4922), a71+b5 (K4923), a71+b6 (K4924), a71+b7 (K4925), a71+b8 (K4926), a71+b9 (K4927), a71+b10 (K4928), a71+b11 (K4929), a71+b12 (K4930), a71+b13 (K4931), a71+b14 (K4932), a71+b15 (K4933), a71+b16 (K4934), a71+b17 (K4935), a71+b18 (K4936), a71+b19 (K4937), a71+b20 (K4938), a71+b21 (K4939), a71+b22 (K4940), a71+b23 (K4941), a71+b24 (K4942), a71+b25 (K4943), a72+b1 (K4944), a72+b2 (K4945), a72+b3 (K4946), a72+b4 (K4947), a72+b5 (K4948), a72+b6 (K4949), a72+b7 (K4950), a72+b8 (K4951), a72+b9 (K4952), a72+b10 (K4953), a72+b11 (K4954), a72+b12 (K4955), a72+b13 (K4956), a72+b14 (K4957), a72+b15 (K4958), a72+b16 (K4959), a72+b17 (K4960), a72+b18 (K4961), a72+b19 (K4962), a72+b20 (K4963), a72+b21 (K4964), a72+b22 (K4965), a72+b23 (K4966), a72+b24 (K4967), a72+b25 (K4968), a73+b1 (K4969), a73+b2 (K4970), a73+b3 (K4971), a73+b4 (K4972), a73+b5 (K4973), a73+b6 (K4974), a73+b7 (K4975), a73+b8 (K4976), a73+b9 (K4977), a73+b10 (K4978), a73+b11 (K4979), a73+b12 (K4980), a73+b13 (K4981), a73+b14 (K4982), a73+b15 (K4983), a73+b16 (K4984), a73+b17 (K4985), a73+b18 (K4986), a73+b19 (K4987), a73+b20 (K4988), a73+b21 (K4989), a73+b22 (K4990), a73+b23 (K4991), a73+b24 (K4992), a73+b25 (K4993), a74+b1 (K4994), a74+b2 (K4995), a74+b3 (K4996), a74+b4 (K4997), a74+b5 (K4998), a74+b6 (K4999), a74+b7 (K5000), a74+b8 (K5001), a74+b9 (K5002), a74+b10 (K5003), a74+b11 (K5004), a74+b12 (K5005), a74+b13 (K5006), a74+b14 (K5007), a74+b15 (K5008), a74+b16 (K5009), a74+b17 (K5010), a74+b18 (K5011), a74+b19 (K5012), a74+b20 (K5013), a74+b21 (K5014), a74+b22 (K5015), a74+b23 (K5016), a74+b24 (K5017), a74+b25 (K5018), a75+b1 (K5019), a75+b2 (K5020), a75+b3 (K5021), a75+b4 (K5022), a75+b5 (K5023), a75+b6 (K5024), a75+b7 (K5025), a75+b8 (K5026), a75+b9 (K5027), a75+b10 (K5028), a75+b11 (K5029), a75+b12 (K5030), a75+b13 (K5031), a75+b14 (K5032), a75+b15 (K5033), a75+b16 (K5034), a75+b17 (K5035), a75+b18 (K5036), a75+b19 (K5037), a75+b20 (K5038), a75+b21 (K5039), a75+b22 (K5040), a75+b23 (K5041), a75+b24 (K5042), a75+b25 (K5043), a76+b1 (K5044), a76+b2 (K5045), a76+b3 (K5046), a76+b4 (K5047), a76+b5 (K5048), a76+b6 (K5049), a76+b7 (K5050), a76+b8 (K5051), a76+b9 (K5052), a76+b10 (K5053), a76+b11 (K5054), a76+b12 (K5055), a76+b13 (K5056), a76+b14 (K5057), a76+b15 (K5058), a76+b16 (K5059), a76+b17 (K5060), a76+b18 (K5061), a76+b19 (K5062), a76+b20 (K5063), a76+b21 (K5064), a76+b22 (K5065), a76+b23 (K5066), a76+b24 (K5067), a76+b25 (K5068), a77+b1 (K5069), a77+b2 (K5070), a77+b3 (K5071), a77+b4 (K5072), a77+b5 (K5073), a77+b6 (K5074), a77+b7 (K5075), a77+b8 (K5076), a77+b9 (K5077), a77+b10 (K5078), a77+b11 (K5079), a77+b12 (K5080), a77+b13 (K5081), a77+b14 (K5082), a77+b15 (K5083), a77+b16 (K5084), a77+b17 (K5085), a77+b18 (K5086), a77+b19 (K5087), a77+b20 (K5088), a77+b21 (K5089), a77+b22 (K5090), a77+b23 (K5091), a77+b24 (K5092), a77+b25 (K5093), a78+b1 (K5094), a78+b2 (K5095), a78+b3 (K5096), a78+b4 (K5097), a78+b5 (K5098), a78+b6 (K5099), a78+b7 (K5100), a78+b8 (K5101), a78+b9 (K5102), a78+b10 (K5103), a78+b11 (K5104), a78+b12 (K5105), a78+b13 (K5106), a78+b14 (K5107), a78+b15 (K5108), a78+b16 (K5109), a78+b17 (K5110), a78+b18 (K5111), a78+b19 (K5112), a78+b20 (K5113), a78+b21 (K5114), a78+b22 (K5115), a78+b23 (K5116), a78+b24 (K5117), a78+b25 (K5118), a79+b1 (K5119), a79+b2 (K5120), a79+b3 (K5121), a79+b4 (K5122), a79+b5 (K5123), a79+b6 (K5124), a79+b7 (K5125), a79+b8 (K5126), a79+b9 (K5127), a79+b10 (K5128), a79+b11 (K5129), a79+b12 (K5130), a79+b13 (K5131), a79+b14 (K5132), a79+b15 (K5133), a79+b16 (K5134), a79+b17 (K5135), a79+b18 (K5136), a79+b19 (K5137), a79+b20 (K5138), a79+b21 (K5139), a79+b22 (K5140), a79+b23 (K5141), a79+b24 (K5142), a79+b25 (K5143), a80+b1 (K5144), a80+b2 (K5145), a80+b3 (K5146), a80+b4 (K5147), a80+b5 (K5148), a80+b6 (K5149), a80+b7 (K5150), a80+b8 (K5151), a80+b9 (K5152), a80+b10 (K5153), a80+b11 (K5154), a80+b12 (K5155), a80+b13 (K5156), a80+b14 (K5157), a80+b15 (K5158), a80+b16 (K5159), a80+b17 (K5160), a80+b18 (K5161), a80+b19 (K5162), a80+b20 (K5163), a80+b21 (K5164), a80+b22 (K5165), a80+b23 (K5166), a80+b24 (K5167), a80+b25 (K5168), a81+b1 (K5169), a81+b2 (K5170), a81+b3 (K5171), a81+b4 (K5172), a81+b5 (K5173), a81+b6 (K5174), a81+b7 (K5175), a81+b8 (K5176), a81+b9 (K5177), a81+b10 (K5178), a81+b11 (K5179), a81+b12 (K5180), a81+b13 (K5181), a81+b14 (K5182), a81+b15 (K5183), a81+b16 (K5184), a81+b17 (K5185), a81+b18 (K5186), a81+b19 (K5187), a81+b20 (K5188), a81+b21 (K5189), a81+b22 (K5190), a81+b23 (K5191), a81+b24 (K5192), a81+b25 (K5193), a82+b1 (K5194), a82+b2 (K5195), a82+b3 (K5196), a82+b4 (K5197), a82+b5 (K5198), a82+b6 (K5199), a82+b7 (K5200), a82+b8 (K5201), a82+b9 (K5202), a82+b10 (K5203), a82+b11 (K5204), a82+b12 (K5205), a82+b13 (K5206), a82+b14 (K5207), a82+b15 (K5208), a82+b16 (K5209), a82+b17 (K5210), a82+b18 (K5211), a82+b19 (K5212), a82+b20 (K5213), a82+b21 (K5214), a82+b22 (K5215), a82+b23 (K5216), a82+b24 (K5217), a82+b25 (K5218), a83+b1 (K5219), a83+b2 (K5220), a83+b3 (K5221), a83+b4 (K5222), a83+b5 (K5223), a83+b6 (K5224), a83+b7 (K5225), a83+b8 (K5226), a83+b9 (K5227), a83+b10 (K5228), a83+b11 (K5229), a83+b12 (K5230), a83+b13 (K5231), a83+b14 (K5232), a83+b15 (K5233), a83+b16 (K5234), a83+b17 (K5235), a83+b18 (K5236), a83+b19 (K5237), a83+b20 (K5238), a83+b21 (K5239), a83+b22 (K5240), a83+b23 (K5241), a83+b24 (K5242), a83+b25 (K5243), a84+b1 (K5244), a84+b2 (K5245), a84+b3 (K5246), a84+b4 (K5247), a84+b5 (K5248), a84+b6 (K5249), a84+b7 (K5250), a84+b8 (K5251), a84+b9 (K5252), a84+b10 (K5253), a84+b11 (K5254), a84+b12 (K5255), a84+b13 (K5256), a84+b14 (K5257), a84+b15 (K5258), a84+b16 (K5259), a84+b17 (K5260), a84+b18 (K5261), a84+b19 (K5262), a84+b20 (K5263), a84+b21 (K5264), a84+b22 (K5265), a84+b23 (K5266), a84+b24 (K5267), a84+b25 (K5268), a85+b1 (K5269), a85+b2 (K5270), a85+b3 (K5271), a85+b4 (K5272), a85+b5 (K5273), a85+b6 (K5274), a85+b7 (K5275), a85+b8 (K5276), a85+b9 (K5277), a85+b10 (K5278), a85+b11 (K5279), a85+b12 (K5280), a85+b13 (K5281), a85+b14 (K5282), a85+b15 (K5283), a85+b16 (K5284), a85+b17 (K5285), a85+b18 (K5286), a85+b19 (K5287), a85+b20 (K5288), a85+b21 (K5289), a85+b22 (K5290), a85+b23 (K5291), a85+b24 (K5292), a85+b25 (K5293), a86+b1 (K5294), a86+b2 (K5295), a86+b3 (K5296), a86+b4 (K5297), a86+b5 (K5298), a86+b6 (K5299), a86+b7 (K5300), a86+b8 (K5301), a86+b9 (K5302), a86+b10 (K5303), a86+b11 (K5304), a86+b12 (K5305), a86+b13 (K5306), a86+b14 (K5307), a86+b15 (K5308), a86+b16 (K5309), a86+b17 (K5310), a86+b18 (K5311), a86+b19 (K5312), a86+b20 (K5313), a86+b21 (K5314), a86+b22 (K5315), a86+b23 (K5316), a86+b24 (K5317), a86+b25 (K5318), a87+b1 (K5319), a87+b2 (K5320), a87+b3 (K5321), a87+b4 (K5322), a87+b5 (K5323), a87+b6 (K5324), a87+b7 (K5325), a87+b8 (K5326), a87+b9 (K5327), a87+b10 (K5328), a87+b11 (K5329), a87+b12 (K5330), a87+b13 (K5331), a87+b14 (K5332), a87+b15 (K5333), a87+b16 (K5334), a87+b17 (K5335), a87+b18 (K5336), a87+b19 (K5337), a87+b20 (K5338), a87+b21 (K5339), a87+b22 (K5340), a87+b23 (K5341), a87+b24 (K5342), a87+b25 (K5343), a88+b1 (K5344), a88+b2 (K5345), a88+b3 (K5346), a88+b4 (K5347), a88+b5 (K5348), a88+b6 (K5349), a88+b7 (K5350), a88+b8 (K5351), a88+b9 (K5352), a88+b10 (K5353), a88+b11 (K5354), a88+b12 (K5355), a88+b13 (K5356), a88+b14 (K5357), a88+b15 (K5358), a88+b16 (K5359), a88+b17 (K5360), a88+b18 (K5361), a88+b19 (K5362), a88+b20 (K5363), a88+b21 (K5364), a88+b22 (K5365), a88+b23 (K5366), a88+b24 (K5367), a88+b25 (K5368), a89+b1 (K5369), a89+b2 (K5370), a89+b3 (K5371), a89+b4 (K5372), a89+b5 (K5373), a89+b6 (K5374), a89+b7 (K5375), a89+b8 (K5376), a89+b9 (K5377), a89+b10 (K5378), a89+b11 (K5379), a89+b12 (K5380), a89+b13 (K5381), a89+b14 (K5382), a89+b15 (K5383), a89+b16 (K5384), a89+b17 (K5385), a89+b18 (K5386), a89+b19 (K5387), a89+b20 (K5388), a89+b21 (K5389), a89+b22 (K5390), a89+b23 (K5391), a89+b24 (K5392), a89+b25 (K5393), a90+b1 (K5394), a90+b2 (K5395), a90+b3 (K5396), a90+b4 (K5397), a90+b5 (K5398), a90+b6 (K5399), a90+b7 (K5400), a90+b8 (K5401), a90+b9 (K5402), a90+b10 (K5403), a90+b11 (K5404), a90+b12 (K5405), a90+b13 (K5406), a90+b14 (K5407), a90+b15 (K5408), a90+b16 (K5409), a90+b17 (K5410), a90+b18 (K5411), a90+b19 (K5412), a90+b20 (K5413), a90+b21 (K5414), a90+b22 (K5415), a90+b23 (K5416), a90+b24 (K5417), a90+b25 (K5418), a91+b1 (K5419), a91+b2 (K5420), a91+b3 (K5421), a91+b4 (K5422), a91+b5 (K5423), a91+b6 (K5424), a91+b7 (K5425), a91+b8 (K5426), a91+b9 (K5427), a91+b10 (K5428), a91+b11 (K5429), a91+b12 (K5430), a91+b13 (K5431), a91+b14 (K5432), a91+b15 (K5433), a91+b16 (K5434), a91+b17 (K5435), a91+b18 (K5436), a91+b19 (K5437), a91+b20 (K5438), a91+b21 (K5439), a91+b22 (K5440), a91+b23 (K5441), a91+b24 (K5442), a91+b25 (K5443), a92+b1 (K5444), a92+b2 (K5445), a92+b3 (K5446), a92+b4 (K5447), a92+b5 (K5448), a92+b6 (K5449), a92+b7 (K5450), a92+b8 (K5451), a92+b9 (K5452), a92+b10 (K5453), a92+b11 (K5454), a92+b12 (K5455), a92+b13 (K5456), a92+b14 (K5457), a92+b15 (K5458), a92+b16 (K5459), a92+b17 (K5460), a92+b18 (K5461), a92+b19 (K5462), a92+b20 (K5463), a92+b21 (K5464), a92+b22 (K5465), a92+b23 (K5466), a92+b24 (K5467), a92+b25 (K5468), a93+b1 (K5469), a93+b2 (K5470), a93+b3 (K5471), a93+b4 (K5472), a93+b5 (K5473), a93+b6 (K5474), a93+b7 (K5475), a93+b8 (K5476), a93+b9 (K5477), a93+b10 (K5478), a93+b11 (K5479), a93+b12 (K5480), a93+b13 (K5481), a93+b14 (K5482), a93+b15 (K5483), a93+b16 (K5484), a93+b17 (K5485), a93+b18 (K5486), a93+b19 (K5487), a93+b20 (K5488), a93+b21 (K5489), a93+b22 (K5490), a93+b23 (K5491), a93+b24 (K5492), a93+b25 (K5493), a94+b1 (K5494), a94+b2 (K5495), a94+b3 (K5496), a94+b4 (K5497), a94+b5 (K5498), a94+b6 (K5499), a94+b7 (K5500), a94+b8 (K5501), a94+b9 (K5502), a94+b10 (K5503), a94+b11 (K5504), a94+b12 (K5505), a94+b13 (K5506), a94+b14 (K5507), a94+b15 (K5508), a94+b16 (K5509), a94+b17 (K5510), a94+b18 (K5511), a94+b19 (K5512), a94+b20 (K5513), a94+b21 (K5514), a94+b22 (K5515), a94+b23 (K5516), a94+b24 (K5517), a94+b25 (K5518), a95+b1 (K5519), a95+b2 (K5520), a95+b3 (K5521), a95+b4 (K5522), a95+b5 (K5523), a95+b6 (K5524), a95+b7 (K5525), a95+b8 (K5526), a95+b9 (K5527), a95+b10 (K5528), a95+b11 (K5529), a95+b12 (K5530), a95+b13 (K5531), a95+b14 (K5532), a95+b15 (K5533), a95+b16 (K5534), a95+b17 (K5535), a95+b18 (K5536), a95+b19 (K5537), a95+b20 (K5538), a95+b21 (K5539), a95+b22 (K5540), a95+b23 (K5541), a95+b24 (K5542), a95+b25 (K5543), a96+b1 (K5544), a96+b2 (K5545), a96+b3 (K5546), a96+b4 (K5547), a96+b5 (K5548), a96+b6 (K5549), a96+b7 (K5550), a96+b8 (K5551), a96+b9 (K5552), a96+b10 (K5553), a96+b11 (K5554), a96+b12 (K5555), a96+b13 (K5556), a96+b14 (K5557), a96+b15 (K5558), a96+b16 (K5559), a96+b17 (K5560), a96+b18 (K5561), a96+b19 (K5562), a96+b20 (K5563), a96+b21 (K5564), a96+b22 (K5565), a96+b23 (K5566), a96+b24 (K5567), a96+b25 (K5568), a97+b1 (K5569), a97+b2 (K5570), a97+b3 (K5571), a97+b4 (K5572), a97+b5 (K5573), a97+b6 (K5574), a97+b7 (K5575), a97+b8 (K5576), a97+b9 (K5577), a97+b10 (K5578), a97+b11 (K5579), a97+b12 (K5580), a97+b13 (K5581), a97+b14 (K5582), a97+b15 (K5583), a97+b16 (K5584), a97+b17 (K5585), a97+b18 (K5586), a97+b19 (K5587), a97+b20 (K5588), a97+b21 (K5589), a97+b22 (K5590), a97+b23 (K5591), a97+b24 (K5592), a97+b25 (K5593), a98+b1 (K5594), a98+b2 (K5595), a98+b3 (K5596), a98+b4 (K5597), a98+b5 (K5598), a98+b6 (K5599), a98+b7 (K5600), a98+b8 (K5601), a98+b9 (K5602), a98+b10 (K5603), a98+b11 (K5604), a98+b12 (K5605), a98+b13 (K5606), a98+b14 (K5607), a98+b15 (K5608), a98+b16 (K5609), a98+b17 (K5610), a98+b18 (K5611), a98+b19 (K5612), a98+b20 (K5613), a98+b21 (K5614), a98+b22 (K5615), a98+b23 (K5616), a98+b24 (K5617), a98+b25 (K5618), a99+b1 (K5619), a99+b2 (K5620), a99+b3 (K5621), a99+b4 (K5622), a99+b5 (K5623), a99+b6 (K5624), a99+b7 (K5625), a99+b8 (K5626), a99+b9 (K5627), a99+b10 (K5628), a99+b11 (K5629), a99+b12 (K5630), a99+b13 (K5631), a99+b14 (K5632), a99+b15 (K5633), a99+b16 (K5634), a99+b17 (K5635), a99+b18 (K5636), a99+b19 (K5637), a99+b20 (K5638), a99+b21 (K5639), a99+b22 (K5640), a99+b23 (K5641), a99+b24 (K5642), a99+b25 (K5643), a100+b1 (K5644), a100+b2 (K5645), a100+b3 (K5646), a100+b4 (K5647), a100+b5 (K5648), a100+b6 (K5649), a100+b7 (K5650), a100+b8 (K5651), a100+b9 (K5652), a100+b10 (K5653), a100+b11 (K5654), a100+b12 (K5655), a100+b13 (K5656), a100+b14 (K5657), a100+b15 (K5658), a100+b16 (K5659), a100+b17 (K5660), a100+b18 (K5661), a100+b19 (K5662), a100+b20 (K5663), a100+b21 (K5664), a100+b22 (K5665), a100+b23 (K5666), a100+b24 (K5667), a100+b25 (K5668), a101+b1 (K5669), a101+b2 (K5670), a101+b3 (K5671), a101+b4 (K5672), a101+b5 (K5673), a101+b6 (K5674), a101+b7 (K5675), a101+b8 (K5676), a101+b9 (K5677), a101+b10 (K5678), a101+b11 (K5679), a101+b12 (K5680), a101+b13 (K5681), a101+b14 (K5682), a101+b15 (K5683), a101+b16 (K5684), a101+b17 (K5685), a101+b18 (K5686), a101+b19 (K5687), a101+b20 (K5688), a101+b21 (K5689), a101+b22 (K5690), a101+b23 (K5691), a101+b24 (K5692), a101+b25 (K5693), a102+b1 (K5694), a102+b2 (K5695), a102+b3 (K5696), a102+b4 (K5697), a102+b5 (K5698), a102+b6 (K5699), a102+b7 (K5700), a102+b8 (K5701), a102+b9 (K5702), a102+b10 (K5703), a102+b11 (K5704), a102+b12 (K5705), a102+b13 (K5706), a102+b14 (K5707), a102+b15 (K5708), a102+b16 (K5709), a102+b17 (K5710), a102+b18 (K5711), a102+b19 (K5712), a102+b20 (K5713), a102+b21 (K5714), a102+b22 (K5715), a102+b23 (K5716), a102+b24 (K5717), a102+b25 (K5718), a103+b1 (K5719), a103+b2 (K5720), a103+b3 (K5721), a103+b4 (K5722), a103+b5 (K5723), a103+b6 (K5724), a103+b7 (K5725), a103+b8 (K5726), a103+b9 (K5727), a103+b10 (K5728), a103+b11 (K5729), a103+b12 (K5730), a103+b13 (K5731), a103+b14 (K5732), a103+b15 (K5733), a103+b16 (K5734), a103+b17 (K5735), a103+b18 (K5736), a103+b19 (K5737), a103+b20 (K5738), a103+b21 (K5739), a103+b22 (K5740), a103+b23 (K5741), a103+b24 (K5742), a103+b25 (K5743), a104+b1 (K5744), a104+b2 (K5745), a104+b3 (K5746), a104+b4 (K5747), a104+b5 (K5748), a104+b6 (K5749), a104+b7 (K5750), a104+b8 (K5751), a104+b9 (K5752), a104+b10 (K5753), a104+b11 (K5754), a104+b12 (K5755), a104+b13 (K5756), a104+b14 (K5757), a104+b15 (K5758), a104+b16 (K5759), a104+b17 (K5760), a104+b18 (K5761), a104+b19 (K5762), a104+b20 (K5763), a104+b21 (K5764), a104+b22 (K5765), a104+b23 (K5766), a104+b24 (K5767), and a104+b25 (K5768), and wherein the at least one module (a), the at least one module (b), and the at least one module (c), and the at least one compound (d) are linked to each other in any arrangement and stoichiometry.

Preferably, the conjugate comprises, essentially consists of, consists of or contains:

(a) at least one module (a) that mediates cell targeting and facilitates cellular uptake,
b) at least one module (b) that facilitates transport to the ER,
(c) at least one module (c) that mediates translocation from the ER to the cytosol, and
(d) at least one compound (d),
wherein the at least one module (a) and the at least one module (c) are selected from the group of combinations consisting of a1+c1, a1+c2, a1+c3, a1+c4, a1+c5, a1+c6, a1+c7, a1+c8, a1+c9, a1+c10, a1+c11, a1+c12, a1+c13, a1+c14, a1+c15, a1+c16, a1+c17, a1+c18, a1+c19, a1+c20, a1+c21, a1+c22, a1+c23, a1+c24, a1+c25, a1+c26, a1+c27, a1+c28, a1+c29, a1+c30, a1+c31, a1+c32, a1+c33, a1+c34, a1+c35, a1+c36, a1+c37, a1+c38, a1+c39, a1+c40, a1+c41, a1+c42, a1+c43, a1+c44, a1+c45, a1+c46, a1+c47, a1+c48, a1+c49, a1+c50, a1+c51, a1+c52, a1+c53, a1+c54, a1+c55, a1+c56, a1+c57, a1+c58, a1+c59, a1+c60, a1+c61, a1+c62, a1+c63, a1+c64, a1+c65, a1+c66, a1+c67, a1+c68, a1+c69, a1+c70, a1+c71, a1+c72, a1+c73, a1+c74, a1+c75, a1+c76, a1+c77, a1+c78, a1+c79, a1+c80, a1+c81, a1+c82, a1+c83, a1+c84, a1+c85, a1+c86, a1+c87, a1+c88, a1+c89, a1+c90, a1+c91, a1+c92, a1+c93, a1+c94, a1+c95, a1+c96, a1+c97, a1+c98, a1+c99, a1+c100, a1+c101, a1+c102, a1+c103, a1+c104, a1+c105, a1+c106, a1+c107, a1+c108, a1+c109, a1+c110, a1+c111, a1+c112, a1+c113, a1+c114, a1+c115, a1+c116, a1+c117, a1+c118, a1+c119, a1+c120, a1+c121, a1+c122, a1+c123, a1+c124, a1+c125, a1+c126, a1+c127, a1+c128, a1+c129, a1+c130, a1+c131, a1+c132, a1+c133, a1+c134, a1+c135, a1+c136, a1+c137, a1+c138, a1+c139, a1+c140, a1+c141, a1+c142, a1+c143, a1+c144, a1+c145, a1+c146, a1+c147, a1+c148, a1+c149, a1+c150, a1+c151, a1+c152, a1+c153, a1+c154, a1+c155, a1+c156, a1+c157, a1+c158, a1+c159, a1+c160, a1+c161, a1+c162, a1+c163, a1+c164, a1+c165, a1+c166, a1+c167, a1+c168, a1+c169, a1+c170, a1+c171, a1+c172, a1+c173, a1+c174, a1+c175, a1+c176, a1+c177, a1+c178, a1+c179, a1+c180, a1+c181, a1+c182, a1+c183, a1+c184, a1+c185, a1+c186, a1+c187, a1+c188, a1+c189, a1+c190, a1+c191, a1+c192, a1+c193, a1+c194, a1+c195, a1+c196, a1+c197, a1+c198, a1+c199, a1+c200, a1+c201, a1+c202, a1+c203, a1+c204, a1+c205, a1+c206, a1+c207, a1+c208, a1+c209, a1+c210, a1+c211, a2+c1, a2+c2, a2+c3, a2+c4, a2+c5, a2+c6, a2+c7, a2+c8, a2+c9, a2+c10, a2+c11, a2+c12, a2+c13, a2+c14, a2+c15, a2+c16, a2+c17, a2+c18, a2+c19, a2+c20, a2+c21, a2+c22, a2+c23, a2+c24, a2+c25, a2+c26, a2+c27, a2+c28, a2+c29, a2+c30, a2+c31, a2+c32, a2+c33, a2+c34, a2+c35, a2+c36, a2+c37, a2+c38, a2+c39, a2+c40, a2+c41, a2+c42, a2+c43, a2+c44, a2+c45, a2+c46, a2+c47, a2+c48, a2+c49, a2+c50, a2+c51, a2+c52, a2+c53, a2+c54, a2+c55, a2+c56, a2+c57, a2+c58, a2+c59, a2+c60, a2+c61, a2+c62, a2+c63, a2+c64, a2+c65, a2+c66, a2+c67, a2+c68, a2+c69, a2+c70, a2+c71, a2+c72, a2+c73, a2+c74, a2+c75, a2+c76, a2+c77, a2+c78, a2+c79, a2+c80, a2+c81, a2+c82, a2+c83, a2+c84, a2+c85, a2+c86, a2+c87, a2+c88, a2+c89, a2+c90, a2+c91, a2+c92, a2+c93, a2+c94, a2+c95, a2+c96, a2+c97, a2+c98, a2+c99, a2+c100, a2+c101, a2+c102, a2+c103, a2+c104, a2+c105, a2+c106, a2+c107, a2+c108, a2+c109, a2+c110, a2+c111, a2+c112, a2+c113, a2+c114, a2+c115, a2+c116, a2+c117, a2+c118, a2+c119, a2+c120, a2+c121, a2+c122, a2+c123, a2+c124, a2+c125, a2+c126, a2+c127, a2+c128, a2+c129, a2+c130, a2+c131, a2+c132, a2+c133, a2+c134, a2+c135, a2+c136, a2+c137, a2+c138, a2+c139, a2+c140, a2+c141, a2+c142, a2+c143, a2+c144, a2+c145, a2+c146, a2+c147, a2+c148, a2+c149, a2+c150, a2+c151, a2+c152, a2+c153, a2+c154, a2+c155, a2+c156, a2+c157, a2+c158, a2+c159, a2+c160, a2+c161, a2+c162, a2+c163, a2+c164, a2+c165, a2+c166, a2+c167, a2+c168, a2+c169, a2+c170, a2+c171, a2+c172, a2+c173, a2+c174, a2+c175, a2+c176, a2+c177, a2+c178, a2+c179, a2+c180, a2+c181, a2+c182, a2+c183, a2+c184, a2+c185, a2+c186, a2+c187, a2+c188, a2+c189, a2+c190, a2+c191, a2+c192, a2+c193, a2+c194, a2+c195, a2+c196, a2+c197, a2+c198, a2+c199, a2+c200, a2+c201, a2+c202, a2+c203, a2+c204, a2+c205, a2+c206, a2+c207, a2+c208, a2+c209, a2+c210, a2+c211, a3+c1, a3+c2, a3+c3, a3+c4, a3+c5, a3+c6, a3+c7, a3+c8, a3+c9, a3+c10, a3+c11, a3+c12, a3+c13, a3+c14, a3+c15, a3+c16, a3+c17, a3+c18, a3+c19, a3+c20, a3+c21, a3+c22, a3+c23, a3+c24, a3+c25, a3+c26, a3+c27, a3+c28, a3+c29, a3+c30, a3+c31, a3+c32, a3+c33, a3+c34, a3+c35, a3+c36, a3+c37, a3+c38, a3+c39, a3+c40, a3+c41, a3+c42, a3+c43, a3+c44, a3+c45, a3+c46, a3+c47, a3+c48, a3+c49, a3+c50, a3+c51, a3+c52, a3+c53, a3+c54, a3+c55, a3+c56, a3+c57, a3+c58, a3+c59, a3+c60, a3+c61, a3+c62, a3+c63, a3+c64, a3+c65, a3+c66, a3+c67, a3+c68, a3+c69, a3+c70, a3+c71, a3+c72, a3+c73, a3+c74, a3+c75, a3+c76, a3+c77, a3+c78, a3+c79, a3+c80, a3+c81, a3+c82, a3+c83, a3+c84, a3+c85, a3+c86, a3+c87, a3+c88, a3+c89, a3+c90, a3+c91, a3+c92, a3+c93, a3+c94, a3+c95, a3+c96, a3+c97, a3+c98, a3+c99, a3+c100, a3+c101, a3+c102, a3+c103, a3+c104, a3+c105, a3+c106, a3+c107, a3+c108, a3+c109, a3+c110, a3+c111, a3+c112, a3+c113, a3+c114, a3+c115, a3+c116, a3+c117, a3+c118, a3+c119, a3+c120, a3+c121, a3+c122, a3+c123, a3+c124, a3+c125, a3+c126, a3+c127, a3+c128, a3+c129, a3+c130, a3+c131, a3+c132, a3+c133, a3+c134, a3+c135, a3+c136, a3+c137, a3+c138, a3+c139, a3+c140, a3+c141, a3+c142, a3+c143, a3+c144, a3+c145, a3+c146, a3+c147, a3+c148, a3+c149, a3+c150, a3+c151, a3+c152, a3+c153, a3+c154, a3+c155, a3+c156, a3+c157, a3+c158, a3+c159, a3+c160, a3+c161, a3+c162, a3+c163, a3+c164, a3+c165, a3+c166, a3+c167, a3+c168, a3+c169, a3+c170, a3+c171, a3+c172, a3+c173, a3+c174, a3+c175, a3+c176, a3+c177, a3+c178, a3+c179, a3+c180, a3+c181, a3+c182, a3+c183, a3+c184, a3+c185, a3+c186, a3+c187, a3+c188, a3+c189, a3+c190, a3+c191, a3+c192, a3+c193, a3+c194, a3+c195, a3+c196, a3+c197, a3+c198, a3+c199, a3+c200, a3+c201, a3+c202, a3+c203, a3+c204, a3+c205, a3+c206, a3+c207, a3+c208, a3+c209, a3+c210, a3+c211, a4+c1, a4+c2, a4+c3, a4+c4, a4+c5, a4+c6, a4+c7, a4+c8, a4+c9, a4+c10, a4+c11, a4+c12, a4+c13, a4+c14, a4+c15, a4+c16, a4+c17, a4+c18, a4+c19, a4+c20, a4+c21, a4+c22, a4+c23, a4+c24, a4+c25, a4+c26, a4+c27, a4+c28, a4+c29, a4+c30, a4+c31, a4+c32, a4+c33, a4+c34, a4+c35, a4+c36, a4+c37, a4+c38, a4+c39, a4+c40, a4+c41, a4+c42, a4+c43, a4+c44, a4+c45, a4+c46, a4+c47, a4+c48, a4+c49, a4+c50, a4+c51, a4+c52, a4+c53, a4+c54, a4+c55, a4+c56, a4+c57, a4+c58, a4+c59, a4+c60, a4+c61, a4+c62, a4+c63, a4+c64, a4+c65, a4+c66, a4+c67, a4+c68, a4+c69, a4+c70, a4+c71, a4+c72, a4+c73, a4+c74, a4+c75, a4+c76, a4+c77, a4+c78, a4+c79, a4+c80, a4+c81, a4+c82, a4+c83, a4+c84, a4+c85, a4+c86, a4+c87, a4+c88, a4+c89, a4+c90, a4+c91, a4+c92, a4+c93, a4+c94, a4+c95, a4+c96, a4+c97, a4+c98, a4+c99, a4+c100, a4+c101, a4+c102, a4+c103, a4+c104, a4+c105, a4+c106, a4+c107, a4+c108, a4+c109, a4+c110, a4+c111, a4+c112, a4+c113, a4+c114, a4+c115, a4+c116, a4+c117, a4+c118, a4+c119, a4+c120, a4+c121, a4+c122, a4+c123, a4+c124, a4+c125, a4+c126, a4+c127, a4+c128, a4+c129, a4+c130, a4+c131, a4+c132, a4+c133, a4+c134, a4+c135, a4+c136, a4+c137, a4+c138, a4+c139, a4+c140, a4+c141, a4+c142, a4+c143, a4+c144, a4+c145, a4+c146, a4+c147, a4+c148, a4+c149, a4+c150, a4+c151, a4+c152, a4+c153, a4+c154, a4+c155, a4+c156, a4+c157, a4+c158, a4+c159, a4+c160, a4+c161, a4+c162, a4+c163, a4+c164, a4+c165, a4+c166, a4+c167, a4+c168, a4+c169, a4+c170, a4+c171, a4+c172, a4+c173, a4+c174, a4+c175, a4+c176, a4+c177, a4+c178, a4+c179, a4+c180, a4+c181, a4+c182, a4+c183, a4+c184, a4+c185, a4+c186, a4+c187, a4+c188, a4+c189, a4+c190, a4+c191, a4+c192, a4+c193, a4+c194, a4+c195, a4+c196, a4+c197, a4+c198, a4+c199, a4+c200, a4+c201, a4+c202, a4+c203, a4+c204, a4+c205, a4+c206, a4+c207, a4+c208, a4+c209, a4+c210, a4+c211, a5+c1, a5+c2, a5+c3, a5+c4, a5+c5, a5+c6, a5+c7, a5+c8, a5+c9, a5+c10, a5+c11, a5+c12, a5+c13, a5+c14, a5+c15, a5+c16, a5+c17, a5+c18, a5+c19, a5+c20, a5+c21, a5+c22, a5+c23, a5+c24, a5+c25, a5+c26, a5+c27, a5+c28, a5+c29, a5+c30, a5+c31, a5+c32, a5+c33, a5+c34, a5+c35, a5+c36, a5+c37, a5+c38, a5+c39, a5+c40, a5+c41, a5+c42, a5+c43, a5+c44, a5+c45, a5+c46, a5+c47, a5+c48, a5+c49, a5+c50, a5+c51, a5+c52, a5+c53, a5+c54, a5+c55, a5+c56, a5+c57, a5+c58, a5+c59, a5+c60, a5+c61, a5+c62, a5+c63, a5+c64, a5+c65, a5+c66, a5+c67, a5+c68, a5+c69, a5+c70, a5+c71, a5+c72, a5+c73, a5+c74, a5+c75, a5+c76, a5+c77, a5+c78, a5+c79, a5+c80, a5+c81, a5+c82, a5+c83, a5+c84, a5+c85, a5+c86, a5+c87, a5+c88, a5+c89, a5+c90, a5+c91, a5+c92, a5+c93, a5+c94, a5+c95, a5+c96, a5+c97, a5+c98, a5+c99, a5+c100, a5+c101, a5+c102, a5+c103, a5+c104, a5+c105, a5+c106, a5+c107, a5+c108, a5+c109, a5+c110, a5+c111, a5+c112, a5+c113, a5+c114, a5+c115, a5+c116, a5+c117, a5+c118, a5+c119, a5+c120, a5+c121, a5+c122, a5+c123, a5+c124, a5+c125, a5+c126, a5+c127, a5+c128, a5+c129, a5+c130, a5+c131, a5+c132, a5+c133, a5+c134, a5+c135, a5+c136, a5+c137, a5+c138, a5+c139, a5+c140, a5+c141, a5+c142, a5+c143, a5+c144, a5+c145, a5+c146, a5+c147, a5+c148, a5+c149, a5+c150, a5+c151, a5+c152, a5+c153, a5+c154, a5+c155, a5+c156, a5+c157, a5+c158, a5+c159, a5+c160, a5+c161, a5+c162, a5+c163, a5+c164, a5+c165, a5+c166, a5+c167, a5+c168, a5+c169, a5+c170, a5+c171, a5+c172, a5+c173, a5+c174, a5+c175, a5+c176, a5+c177, a5+c178, a5+c179, a5+c180, a5+c181, a5+c182, a5+c183, a5+c184, a5+c185, a5+c186, a5+c187, a5+c188, a5+c189, a5+c190, a5+c191, a5+c192, a5+c193, a5+c194, a5+c195, a5+c196, a5+c197, a5+c198, a5+c199, a5+c200, a5+c201, a5+c202, a5+c203, a5+c204, a5+c205, a5+c206, a5+c207, a5+c208, a5+c209, a5+c210, a5+c211, a6+c1, a6+c2, a6+c3, a6+c4, a6+c5, a6+c6, a6+c7, a6+c8, a6+c9, a6+c10, a6+c11, a6+c12, a6+c13, a6+c14, a6+c15, a6+c16, a6+c17, a6+c18, a6+c19, a6+c20, a6+c21, a6+c22, a6+c23, a6+c24, a6+c25, a6+c26, a6+c27, a6+c28, a6+c29, a6+c30, a6+c31, a6+c32, a6+c33, a6+c34, a6+c35, a6+c36, a6+c37, a6+c38, a6+c39, a6+c40, a6+c41, a6+c42, a6+c43, a6+c44, a6+c45, a6+c46, a6+c47, a6+c48, a6+c49, a6+c50, a6+c51, a6+c52, a6+c53, a6+c54, a6+c55, a6+c56, a6+c57, a6+c58, a6+c59, a6+c60, a6+c61, a6+c62, a6+c63, a6+c64, a6+c65, a6+c66, a6+c67, a6+c68, a6+c69, a6+c70, a6+c71, a6+c72, a6+c73, a6+c74, a6+c75, a6+c76, a6+c77, a6+c78, a6+c79, a6+c80, a6+c81, a6+c82, a6+c83, a6+c84, a6+c85, a6+c86, a6+c87, a6+c88, a6+c89, a6+c90, a6+c91, a6+c92, a6+c93, a6+c94, a6+c95, a6+c96, a6+c97, a6+c98, a6+c99, a6+c100, a6+c101, a6+c102, a6+c103, a6+c104, a6+c105, a6+c106, a6+c107, a6+c108, a6+c109, a6+c110, a6+c111, a6+c112, a6+c113, a6+c114, a6+c115, a6+c116, a6+c117, a6+c118, a6+c119, a6+c120, a6+c121, a6+c122, a6+c123, a6+c124, a6+c125, a6+c126, a6+c127, a6+c128, a6+c129, a6+c130, a6+c131, a6+c132, a6+c133, a6+c134, a6+c135, a6+c136, a6+c137, a6+c138, a6+c139, a6+c140, a6+c141, a6+c142, a6+c143, a6+c144, a6+c145, a6+c146, a6+c147, a6+c148, a6+c149, a6+c150, a6+c151, a6+c152, a6+c153, a6+c154, a6+c155, a6+c156, a6+c157, a6+c158, a6+c159, a6+c160, a6+c161, a6+c162, a6+c163, a6+c164, a6+c165, a6+c166, a6+c167, a6+c168, a6+c169, a6+c170, a6+c171, a6+c172, a6+c173, a6+c174, a6+c175, a6+c176, a6+c177, a6+c178, a6+c179, a6+c180, a6+c181, a6+c182, a6+c183, a6+c184, a6+c185, a6+c186, a6+c187, a6+c188, a6+c189, a6+c190, a6+c191, a6+c192, a6+c193, a6+c194, a6+c195, a6+c196, a6+c197, a6+c198, a6+c199, a6+c200, a6+c201, a6+c202, a6+c203, a6+c204, a6+c205, a6+c206, a6+c207, a6+c208, a6+c209, a6+c210, a6+c211, a7+c1, a7+c2, a7+c3, a7+c4, a7+c5, a7+c6, a7+c7, a7+c8, a7+c9, a7+c10, a7+c11, a7+c12, a7+c13, a7+c14, a7+c15, a7+c16, a7+c17, a7+c18, a7+c19, a7+c20, a7+c21, a7+c22, a7+c23, a7+c24, a7+c25, a7+c26, a7+c27, a7+c28, a7+c29, a7+c30, a7+c31, a7+c32, a7+c33, a7+c34, a7+c35, a7+c36, a7+c37, a7+c38, a7+c39, a7+c40, a7+c41, a7+c42, a7+c43, a7+c44, a7+c45, a7+c46, a7+c47, a7+c48, a7+c49, a7+c50, a7+c51, a7+c52, a7+c53, a7+c54, a7+c55, a7+c56, a7+c57, a7+c58, a7+c59, a7+c60, a7+c61, a7+c62, a7+c63, a7+c64, a7+c65, a7+c66, a7+c67, a7+c68, a7+c69, a7+c70, a7+c71, a7+c72, a7+c73, a7+c74, a7+c75, a7+c76, a7+c77, a7+c78, a7+c79, a7+c80, a7+c81, a7+c82, a7+c83, a7+c84, a7+c85, a7+c86, a7+c87, a7+c88, a7+c89, a7+c90, a7+c91, a7+c92, a7+c93, a7+c94, a7+c95, a7+c96, a7+c97, a7+c98, a7+c99, a7+c100, a7+c101, a7+c102, a7+c103, a7+c104, a7+c105, a7+c106, a7+c107, a7+c108, a7+c109, a7+c110, a7+c111, a7+c112, a7+c113, a7+c114, a7+c115, a7+c116, a7+c117, a7+c118, a7+c119, a7+c120, a7+c121, a7+c122, a7+c123, a7+c124, a7+c125, a7+c126, a7+c127, a7+c128, a7+c129, a7+c130, a7+c131, a7+c132, a7+c133, a7+c134, a7+c135, a7+c136, a7+c137, a7+c138, a7+c139, a7+c140, a7+c141, a7+c142, a7+c143, a7+c144, a7+c145, a7+c146, a7+c147, a7+c148, a7+c149, a7+c150, a7+c151, a7+c152, a7+c153, a7+c154, a7+c155, a7+c156, a7+c157, a7+c158, a7+c159, a7+c160, a7+c161, a7+c162, a7+c163, a7+c164, a7+c165, a7+c166, a7+c167, a7+c168, a7+c169, a7+c170, a7+c171, a7+c172, a7+c173, a7+c174, a7+c175, a7+c176, a7+c177, a7+c178, a7+c179, a7+c180, a7+c181, a7+c182, a7+c183, a7+c184, a7+c185, a7+c186, a7+c187, a7+c188, a7+c189, a7+c190, a7+c191, a7+c192, a7+c193, a7+c194, a7+c195, a7+c196, a7+c197, a7+c198, a7+c199, a7+c200, a7+c201, a7+c202, a7+c203, a7+c204, a7+c205, a7+c206, a7+c207, a7+c208, a7+c209, a7+c210, a7+c211, a8+c1, a8+c2, a8+c3, a8+c4, a8+c5, a8+c6, a8+c7, a8+c8, a8+c9, a8+c10, a8+c11, a8+c12, a8+c13, a8+c14, a8+c15, a8+c16, a8+c17, a8+c18, a8+c19, a8+c20, a8+c21, a8+c22, a8+c23, a8+c24, a8+c25, a8+c26, a8+c27, a8+c28, a8+c29, a8+c30, a8+c31, a8+c32, a8+c33, a8+c34, a8+c35, a8+c36, a8+c37, a8+c38, a8+c39, a8+c40, a8+c41, a8+c42, a8+c43, a8+c44, a8+c45, a8+c46, a8+c47, a8+c48, a8+c49, a8+c50, a8+c51, a8+c52, a8+c53, a8+c54, a8+c55, a8+c56, a8+c57, a8+c58, a8+c59, a8+c60, a8+c61, a8+c62, a8+c63, a8+c64, a8+c65, a8+c66, a8+c67, a8+c68, a8+c69, a8+c70, a8+c71, a8+c72, a8+c73, a8+c74, a8+c75, a8+c76, a8+c77, a8+c78, a8+c79, a8+c80, a8+c81, a8+c82, a8+c83, a8+c84, a8+c85, a8+c86, a8+c87, a8+c88, a8+c89, a8+c90, a8+c91, a8+c92, a8+c93, a8+c94, a8+c95, a8+c96, a8+c97, a8+c98, a8+c99, a8+c100, a8+c101, a8+c102, a8+c103, a8+c104, a8+c105, a8+c106, a8+c107, a8+c108, a8+c109, a8+c110, a8+c111, a8+c112, a8+c113, a8+c114, a8+c115, a8+c116, a8+c117, a8+c118, a8+c119, a8+c120, a8+c121, a8+c122, a8+c123, a8+c124, a8+c125, a8+c126, a8+c127, a8+c128, a8+c129, a8+c130, a8+c131, a8+c132, a8+c133, a8+c134, a8+c135, a8+c136, a8+c137, a8+c138, a8+c139, a8+c140, a8+c141, a8+c142, a8+c143, a8+c144, a8+c145, a8+c146, a8+c147, a8+c148, a8+c149, a8+c150, a8+c151, a8+c152, a8+c153, a8+c154, a8+c155, a8+c156, a8+c157, a8+c158, a8+c159, a8+c160, a8+c161, a8+c162, a8+c163, a8+c164, a8+c165, a8+c166, a8+c167, a8+c168, a8+c169, a8+c170, a8+c171, a8+c172, a8+c173, a8+c174, a8+c175, a8+c176, a8+c177, a8+c178, a8+c179, a8+c180, a8+c181, a8+c182, a8+c183, a8+c184, a8+c185, a8+c186, a8+c187, a8+c188, a8+c189, a8+c190, a8+c191, a8+c192, a8+c193, a8+c194, a8+c195, a8+c196, a8+c197, a8+c198, a8+c199, a8+c200, a8+c201, a8+c202, a8+c203, a8+c204, a8+c205, a8+c206, a8+c207, a8+c208, a8+c209, a8+c210, a8+c211, a9+c1, a9+c2, a9+c3, a9+c4, a9+c5, a9+c6, a9+c7, a9+c8, a9+c9, a9+c10, a9+c11, a9+c12, a9+c13, a9+c14, a9+c15, a9+c16, a9+c17, a9+c18, a9+c19, a9+c20, a9+c21, a9+c22, a9+c23, a9+c24, a9+c25, a9+c26, a9+c27, a9+c28, a9+c29, a9+c30, a9+c31, a9+c32, a9+c33, a9+c34, a9+c35, a9+c36, a9+c37, a9+c38, a9+c39, a9+c40, a9+c41, a9+c42, a9+c43, a9+c44, a9+c45, a9+c46, a9+c47, a9+c48, a9+c49, a9+c50, a9+c51, a9+c52, a9+c53, a9+c54, a9+c55, a9+c56, a9+c57, a9+c58, a9+c59, a9+c60, a9+c61, a9+c62, a9+c63, a9+c64, a9+c65, a9+c66, a9+c67, a9+c68, a9+c69, a9+c70, a9+c71, a9+c72, a9+c73, a9+c74, a9+c75, a9+c76, a9+c77, a9+c78, a9+c79, a9+c80, a9+c81, a9+c82, a9+c83, a9+c84, a9+c85, a9+c86, a9+c87, a9+c88, a9+c89, a9+c90, a9+c91, a9+c92, a9+c93, a9+c94, a9+c95, a9+c96, a9+c97, a9+c98, a9+c99, a9+c100, a9+c101, a9+c102, a9+c103, a9+c104, a9+c105, a9+c106, a9+c107, a9+c108, a9+c109, a9+c110, a9+c111, a9+c112, a9+c113, a9+c114, a9+c115, a9+c116, a9+c117, a9+c118, a9+c119, a9+c120, a9+c121, a9+c122, a9+c123, a9+c124, a9+c125, a9+c126, a9+c127, a9+c128, a9+c129, a9+c130, a9+c131, a9+c132, a9+c133, a9+c134, a9+c135, a9+c136, a9+c137, a9+c138, a9+c139, a9+c140, a9+c141, a9+c142, a9+c143, a9+c144, a9+c145, a9+c146, a9+c147, a9+c148, a9+c149, a9+c150, a9+c151, a9+c152, a9+c153, a9+c154, a9+c155, a9+c156, a9+c157, a9+c158, a9+c159, a9+c160, a9+c161, a9+c162, a9+c163, a9+c164, a9+c165, a9+c166, a9+c167, a9+c168, a9+c169, a9+c170, a9+c171, a9+c172, a9+c173, a9+c174, a9+c175, a9+c176, a9+c177, a9+c178, a9+c179, a9+c180, a9+c181, a9+c182, a9+c183, a9+c184, a9+c185, a9+c186, a9+c187, a9+c188, a9+c189, a9+c190, a9+c191, a9+c192, a9+c193, a9+c194, a9+c195, a9+c196, a9+c197, a9+c198, a9+c199, a9+c200, a9+c201, a9+c202, a9+c203, a9+c204, a9+c205, a9+c206, a9+c207, a9+c208, a9+c209, a9+c210, a9+c211, a10+c1, a10+c2, a10+c3, a10+c4, a10+c5, a10+c6, a10+c7, a10+c8, a10+c9, a10+c10, a10+c11, a10+c12, a10+c13, a10+c14, a10+c15, a10+c16, a10+c17, a10+c18, a10+c19, a10+c20, a10+c21, a10+c22, a10+c23, a10+c24, a10+c25, a10+c26, a10+c27, a10+c28, a10+c29, a10+c30, a10+c31, a10+c32, a10+c33, a10+c34, a10+c35, a10+c36, a10+c37, a10+c38, a10+c39, a10+c40, a10+c41, a10+c42, a10+c43, a10+c44, a10+c45, a10+c46, a10+c47, a10+c48, a10+c49, a10+c50, a10+c51, a10+c52, a10+c53, a10+c54, a10+c55, a10+c56, a10+c57, a10+c58, a10+c59, a10+c60, a10+c61, a10+c62, a10+c63, a10+c64, a10+c65, a10+c66, a10+c67, a10+c68, a10+c69, a10+c70, a10+c71, a10+c72, a10+c73, a10+c74, a10+c75, a10+c76, a10+c77, a10+c78, a10+c79, a10+c80, a10+c81, a10+c82, a10+c83, a10+c84, a10+c85, a10+c86, a10+c87, a10+c88, a10+c89, a10+c90, a10+c91, a10+c92, a10+c93, a10+c94, a10+c95, a10+c96, a10+c97, a10+c98, a10+c99, a10+c100, a10+c101, a10+c102, a10+c103, a10+c104, a10+c105, a10+c106, a10+c107, a10+c108, a10+c109, a10+c110, a10+c111, a10+c112, a10+c113, a10+c114, a10+c115, a10+c116, a10+c117, a10+c118, a10+c119, a10+c120, a10+c121, a10+c122, a10+c123, a10+c124, a10+c125, a10+c126, a10+c127, a10+c128, a10+c129, a10+c130, a10+c131, a10+c132, a10+c133, a10+c134, a10+c135, a10+c136, a10+c137, a10+c138, a10+c139, a10+c140, a10+c141, a10+c142, a10+c143, a10+c144, a10+c145, a10+c146, a10+c147, a10+c148, a10+c149, a10+c150, a10+c151, a10+c152, a10+c153, a10+c154, a10+c155, a10+c156, a10+c157, a10+c158, a10+c159, a10+c160, a10+c161, a10+c162, a10+c163, a10+c164, a10+c165, a10+c166, a10+c167, a10+c168, a10+c169, a10+c170, a10+c171, a10+c172, a10+c173, a10+c174, a10+c175, a10+c176, a10+c177, a10+c178, a10+c179, a10+c180, a10+c181, a10+c182, a10+c183, a10+c184, a10+c185, a10+c186, a10+c187, a10+c188, a10+c189, a10+c190, a10+c191, a10+c192, a10+c193, a10+c194, a10+c195, a10+c196, a10+c197, a10+c198, a10+c199, a10+c200, a10+c201, a10+c202, a10+c203, a10+c204, a10+c205, a10+c206, a10+c207, a10+c208, a10+c209, a10+c210, a10+c211, a11+c1, a11+c2, a11+c3, a11+c4, a11+c5, a11+c6, a11+c7, a11+c8, a11+c9, a11+c10, a11+c11, a11+c12, a11+c13, a11+c14, a11+c15, a11+c16, a11+c17, a11+c18, a11+c19, a11+c20, a11+c21, a11+c22, a11+c23, a11+c24, a11+c25, a11+c26, a11+c27, a11+c28, a11+c29, a11+c30, a11+c31, a11+c32, a11+c33, a11+c34, a11+c35, a11+c36, a11+c37, a11+c38, a11+c39, a11+c40, a11+c41, a11+c42, a11+c43, a11+c44, a11+c45, a11+c46, a11+c47, a11+c48, a11+c49, a11+c50, a11+c51, a11+c52, a11+c53, a11+c54, a11+c55, a11+c56, a11+c57, a11+c58, a11+c59, a11+c60, a11+c61, a11+c62, a11+c63, a11+c64, a11+c65, a11+c66, a11+c67, a11+c68, a11+c69, a11+c70, a11+c71, a11+c72, a11+c73, a11+c74, a11+c75, a11+c76, a11+c77, a11+c78, a11+c79, a11+c80, a11+c81, a11+c82, a11+c83, a11+c84, a11+c85, a11+c86, a11+c87, a11+c88, a11+c89, a11+c90, a11+c91, a11+c92, a11+c93, a11+c94, a11+c95, a11+c96, a11+c97, a11+c98, a11+c99, a11+c100, a11+c101, a11+c102, a11+c103, a11+c104, a11+c105, a11+c106, a11+c107, a11+c108, a11+c109, a11+c110, a11+c111, a11+c112, a11+c113, a11+c114, a11+c115, a11+c116, a11+c117, a11+c118, a11+c119, a11+c120, a11+c121, a11+c122, a11+c123, a11+c124, a11+c125, a11+c126, a11+c127, a11+c128, a11+c129, a11+c130, a11+c131, a11+c132, a11+c133, a11+c134, a11+c135, a11+c136, a11+c137, a11+c138, a11+c139, a11+c140, a11+c141, a11+c142, a11+c143, a11+c144, a11+c145, a11+c146, a11+c147, a11+c148, a11+c149, a11+c150, a11+c151, a11+c152, a11+c153, a11+c154, a11+c155, a11+c156, a11+c157, a11+c158, a11+c159, a11+c160, a11+c161, a11+c162, a11+c163, a11+c164, a11+c165, a11+c166, a11+c167, a11+c168, a11+c169, a11+c170, a11+c171, a11+c172, a11+c173, a11+c174, a11+c175, a11+c176, a11+c177, a11+c178, a11+c179, a11+c180, a11+c181, a11+c182, a11+c183, a11+c184, a11+c185, a11+c186, a11+c187, a11+c188, a11+c189, a11+c190, a11+c191, a11+c192, a11+c193, a11+c194, a11+c195, a11+c196, a11+c197, a11+c198, a11+c199, a11+c200, a11+c201, a11+c202, a11+c203, a11+c204, a11+c205, a11+c206, a11+c207, a11+c208, a11+c209, a11+c210, a11+c211, a12+c1, a12+c2, a12+c3, a12+c4, a12+c5, a12+c6, a12+c7, a12+c8, a12+c9, a12+c10, a12+c11, a12+c12, a12+c13, a12+c14, a12+c15, a12+c16, a12+c17, a12+c18, a12+c19, a12+c20, a12+c21, a12+c22, a12+c23, a12+c24, a12+c25, a12+c26, a12+c27, a12+c28, a12+c29, a12+c30, a12+c31, a12+c32, a12+c33, a12+c34, a12+c35, a12+c36, a12+c37, a12+c38, a12+c39, a12+c40, a12+c41, a12+c42, a12+c43, a12+c44, a12+c45, a12+c46, a12+c47, a12+c48, a12+c49, a12+c50, a12+c51, a12+c52, a12+c53, a12+c54, a12+c55, a12+c56, a12+c57, a12+c58, a12+c59, a12+c60, a12+c61, a12+c62, a12+c63, a12+c64, a12+c65, a12+c66, a12+c67, a12+c68, a12+c69, a12+c70, a12+c71, a12+c72, a12+c73, a12+c74, a12+c75, a12+c76, a12+c77, a12+c78, a12+c79, a12+c80, a12+c81, a12+c82, a12+c83, a12+c84, a12+c85, a12+c86, a12+c87, a12+c88, a12+c89, a12+c90, a12+c91, a12+c92, a12+c93, a12+c94, a12+c95, a12+c96, a12+c97, a12+c98, a12+c99, a12+c100, a12+c101, a12+c102, a12+c103, a12+c104, a12+c105, a12+c106, a12+c107, a12+c108, a12+c109, a12+c110, a12+c111, a12+c112, a12+c113, a12+c114, a12+c115, a12+c116, a12+c117, a12+c118, a12+c119, a12+c120, a12+c121, a12+c122, a12+c123, a12+c124, a12+c125, a12+c126, a12+c127, a12+c128, a12+c129, a12+c130, a12+c131, a12+c132, a12+c133, a12+c134, a12+c135, a12+c136, a12+c137, a12+c138, a12+c139, a12+c140, a12+c141, a12+c142, a12+c143, a12+c144, a12+c145, a12+c146, a12+c147, a12+c148, a12+c149, a12+c150, a12+c151, a12+c152, a12+c153, a12+c154, a12+c155, a12+c156, a12+c157, a12+c158, a12+c159, a12+c160, a12+c161, a12+c162, a12+c163, a12+c164, a12+c165, a12+c166, a12+c167, a12+c168, a12+c169, a12+c170, a12+c171, a12+c172, a12+c173, a12+c174, a12+c175, a12+c176, a12+c177, a12+c178, a12+c179, a12+c180, a12+c181, a12+c182, a12+c183, a12+c184, a12+c185, a12+c186, a12+c187, a12+c188, a12+c189, a12+c190, a12+c191, a12+c192, a12+c193, a12+c194, a12+c195, a12+c196, a12+c197, a12+c198, a12+c199, a12+c200, a12+c201, a12+c202, a12+c203, a12+c204, a12+c205, a12+c206, a12+c207, a12+c208, a12+c209, a12+c210, a12+c211, a13+c1, a13+c2, a13+c3, a13+c4, a13+c5, a13+c6, a13+c7, a13+c8, a13+c9, a13+c10, a13+c11, a13+c12, a13+c13, a13+c14, a13+c15, a13+c16, a13+c17, a13+c18, a13+c19, a13+c20, a13+c21, a13+c22, a13+c23, a13+c24, a13+c25, a13+c26, a13+c27, a13+c28, a13+c29, a13+c30, a13+c31, a13+c32, a13+c33, a13+c34, a13+c35, a13+c36, a13+c37, a13+c38, a13+c39, a13+c40, a13+c41, a13+c42, a13+c43, a13+c44, a13+c45, a13+c46, a13+c47, a13+c48, a13+c49, a13+c50, a13+c51, a13+c52, a13+c53, a13+c54, a13+c55, a13+c56, a13+c57, a13+c58, a13+c59, a13+c60, a13+c61, a13+c62, a13+c63, a13+c64, a13+c65, a13+c66, a13+c67, a13+c68, a13+c69, a13+c70, a13+c71, a13+c72, a13+c73, a13+c74, a13+c75, a13+c76, a13+c77, a13+c78, a13+c79, a13+c80, a13+c81, a13+c82, a13+c83, a13+c84, a13+c85, a13+c86, a13+c87, a13+c88, a13+c89, a13+c90, a13+c91, a13+c92, a13+c93, a13+c94, a13+c95, a13+c96, a13+c97, a13+c98, a13+c99, a13+c100, a13+c101, a13+c102, a13+c103, a13+c104, a13+c105, a13+c106, a13+c107, a13+c108, a13+c109, a13+c110, a13+c111, a13+c112, a13+c113, a13+c114, a13+c115, a13+c116, a13+c117, a13+c118, a13+c119, a13+c120, a13+c121, a13+c122, a13+c123, a13+c124, a13+c125, a13+c126, a13+c127, a13+c128, a13+c129, a13+c130, a13+c131, a13+c132, a13+c133, a13+c134, a13+c135, a13+c136, a13+c137, a13+c138, a13+c139, a13+c140, a13+c141, a13+c142, a13+c143, a13+c144, a13+c145, a13+c146, a13+c147, a13+c148, a13+c149, a13+c150, a13+c151, a13+c152, a13+c153, a13+c154, a13+c155, a13+c156, a13+c157, a13+c158, a13+c159, a13+c160, a13+c161, a13+c162, a13+c163, a13+c164, a13+c165, a13+c166, a13+c167, a13+c168, a13+c169, a13+c170, a13+c171, a13+c172, a13+c173, a13+c174, a13+c175, a13+c176, a13+c177, a13+c178, a13+c179, a13+c180, a13+c181, a13+c182, a13+c183, a13+c184, a13+c185, a13+c186, a13+c187, a13+c188, a13+c189, a13+c190, a13+c191, a13+c192, a13+c193, a13+c194, a13+c195, a13+c196, a13+c197, a13+c198, a13+c199, a13+c200, a13+c201, a13+c202, a13+c203, a13+c204, a13+c205, a13+c206, a13+c207, a13+c208, a13+c209, a13+c210, a13+c211, a14+c1, a14+c2, a14+c3, a14+c4, a14+c5, a14+c6, a14+c7, a14+c8, a14+c9, a14+c10, a14+c11, a14+c12, a14+c13, a14+c14, a14+c15, a14+c16, a14+c17, a14+c18, a14+c19, a14+c20, a14+c21, a14+c22, a14+c23, a14+c24, a14+c25, a14+c26, a14+c27, a14+c28, a14+c29, a14+c30, a14+c31, a14+c32, a14+c33, a14+c34, a14+c35, a14+c36, a14+c37, a14+c38, a14+c39, a14+c40, a14+c41, a14+c42, a14+c43, a14+c44, a14+c45, a14+c46, a14+c47, a14+c48, a14+c49, a14+c50, a14+c51, a14+c52, a14+c53, a14+c54, a14+c55, a14+c56, a14+c57, a14+c58, a14+c59, a14+c60, a14+c61, a14+c62, a14+c63, a14+c64, a14+c65, a14+c66, a14+c67, a14+c68, a14+c69, a14+c70, a14+c71, a14+c72, a14+c73, a14+c74, a14+c75, a14+c76, a14+c77, a14+c78, a14+c79, a14+c80, a14+c81, a14+c82, a14+c83, a14+c84, a14+c85, a14+c86, a14+c87, a14+c88, a14+c89, a14+c90, a14+c91, a14+c92, a14+c93, a14+c94, a14+c95, a14+c96, a14+c97, a14+c98, a14+c99, a14+c100, a14+c101, a14+c102, a14+c103, a14+c104, a14+c105, a14+c106, a14+c107, a14+c108, a14+c109, a14+c110, a14+c111, a14+c112, a14+c113, a14+c114, a14+c115, a14+c116, a14+c117, a14+c118, a14+c119, a14+c120, a14+c121, a14+c122, a14+c123, a14+c124, a14+c125, a14+c126, a14+c127, a14+c128, a14+c129, a14+c130, a14+c131, a14+c132, a14+c133, a14+c134, a14+c135, a14+c136, a14+c137, a14+c138, a14+c139, a14+c140, a14+c141, a14+c142, a14+c143, a14+c144, a14+c145, a14+c146, a14+c147, a14+c148, a14+c149, a14+c150, a14+c151, a14+c152, a14+c153, a14+c154, a14+c155, a14+c156, a14+c157, a14+c158, a14+c159, a14+c160, a14+c161, a14+c162, a14+c163, a14+c164, a14+c165, a14+c166, a14+c167, a14+c168, a14+c169, a14+c170, a14+c171, a14+c172, a14+c173, a14+c174, a14+c175, a14+c176, a14+c177, a14+c178, a14+c179, a14+c180, a14+c181, a14+c182, a14+c183, a14+c184, a14+c185, a14+c186, a14+c187, a14+c188, a14+c189, a14+c190, a14+c191, a14+c192, a14+c193, a14+c194, a14+c195, a14+c196, a14+c197, a14+c198, a14+c199, a14+c200, a14+c201, a14+c202, a14+c203, a14+c204, a14+c205, a14+c206, a14+c207, a14+c208, a14+c209, a14+c210, a14+c211, a15+c1, a15+c2, a15+c3, a15+c4, a15+c5, a15+c6, a15+c7, a15+c8, a15+c9, a15+c10, a15+c11, a15+c12, a15+c13, a15+c14, a15+c15, a15+c16, a15+c17, a15+c18, a15+c19, a15+c20, a15+c21, a15+c22, a15+c23, a15+c24, a15+c25, a15+c26, a15+c27, a15+c28, a15+c29, a15+c30, a15+c31, a15+c32, a15+c33, a15+c34, a15+c35, a15+c36, a15+c37, a15+c38, a15+c39, a15+c40, a15+c41, a15+c42, a15+c43, a15+c44, a15+c45, a15+c46, a15+c47, a15+c48, a15+c49, a15+c50, a15+c51, a15+c52, a15+c53, a15+c54, a15+c55, a15+c56, a15+c57, a15+c58, a15+c59, a15+c60, a15+c61, a15+c62, a15+c63, a15+c64, a15+c65, a15+c66, a15+c67, a15+c68, a15+c69, a15+c70, a15+c71, a15+c72, a15+c73, a15+c74, a15+c75, a15+c76, a15+c77, a15+c78, a15+c79, a15+c80, a15+c81, a15+c82, a15+c83, a15+c84, a15+c85, a15+c86, a15+c87, a15+c88, a15+c89, a15+c90, a15+c91, a15+c92, a15+c93, a15+c94, a15+c95, a15+c96, a15+c97, a15+c98, a15+c99, a15+c100, a15+c101, a15+c102, a15+c103, a15+c104, a15+c105, a15+c106, a15+c107, a15+c108, a15+c109, a15+c110, a15+c111, a15+c112, a15+c113, a15+c114, a15+c115, a15+c116, a15+c117, a15+c118, a15+c119, a15+c120, a15+c121, a15+c122, a15+c123, a15+c124, a15+c125, a15+c126, a15+c127, a15+c128, a15+c129, a15+c130, a15+c131, a15+c132, a15+c133, a15+c134, a15+c135, a15+c136, a15+c137, a15+c138, a15+c139, a15+c140, a15+c141, a15+c142, a15+c143, a15+c144, a15+c145, a15+c146, a15+c147, a15+c148, a15+c149, a15+c150, a15+c151, a15+c152, a15+c153, a15+c154, a15+c155, a15+c156, a15+c157, a15+c158, a15+c159, a15+c160, a15+c161, a15+c162, a15+c163, a15+c164, a15+c165, a15+c166, a15+c167, a15+c168, a15+c169, a15+c170, a15+c171, a15+c172, a15+c173, a15+c174, a15+c175, a15+c176, a15+c177, a15+c178, a15+c179, a15+c180, a15+c181, a15+c182, a15+c183, a15+c184, a15+c185, a15+c186, a15+c187, a15+c188, a15+c189, a15+c190, a15+c191, a15+c192, a15+c193, a15+c194, a15+c195, a15+c196, a15+c197, a15+c198, a15+c199, a15+c200, a15+c201, a15+c202, a15+c203, a15+c204, a15+c205, a15+c206, a15+c207, a15+c208, a15+c209, a15+c210, a15+c211, a16+c1, a16+c2, a16+c3, a16+c4, a16+c5, a16+c6, a16+c7, a16+c8, a16+c9, a16+c10, a16+c11, a16+c12, a16+c13, a16+c14, a16+c15, a16+c16, a16+c17, a16+c18, a16+c19, a16+c20, a16+c21, a16+c22, a16+c23, a16+c24, a16+c25, a16+c26, a16+c27, a16+c28, a16+c29, a16+c30, a16+c31, a16+c32, a16+c33, a16+c34, a16+c35, a16+c36, a16+c37, a16+c38, a16+c39, a16+c40, a16+c41, a16+c42, a16+c43, a16+c44, a16+c45, a16+c46, a16+c47, a16+c48, a16+c49, a16+c50, a16+c51, a16+c52, a16+c53, a16+c54, a16+c55, a16+c56, a16+c57, a16+c58, a16+c59, a16+c60, a16+c61, a16+c62, a16+c63, a16+c64, a16+c65, a16+c66, a16+c67, a16+c68, a16+c69, a16+c70, a16+c71, a16+c72, a16+c73, a16+c74, a16+c75, a16+c76, a16+c77, a16+c78, a16+c79, a16+c80, a16+c81, a16+c82, a16+c83, a16+c84, a16+c85, a16+c86, a16+c87, a16+c88, a16+c89, a16+c90, a16+c91, a16+c92, a16+c93, a16+c94, a16+c95, a16+c96, a16+c97, a16+c98, a16+c99, a16+c100, a16+c101, a16+c102, a16+c103, a16+c104, a16+c105, a16+c106, a16+c107, a16+c108, a16+c109, a16+c110, a16+c111, a16+c112, a16+c113, a16+c114, a16+c115, a16+c116, a16+c117, a16+c118, a16+c119, a16+c120, a16+c121, a16+c122, a16+c123, a16+c124, a16+c125, a16+c126, a16+c127, a16+c128, a16+c129, a16+c130, a16+c131, a16+c132, a16+c133, a16+c134, a16+c135, a16+c136, a16+c137, a16+c138, a16+c139, a16+c140, a16+c141, a16+c142, a16+c143, a16+c144, a16+c145, a16+c146, a16+c147, a16+c148, a16+c149, a16+c150, a16+c151, a16+c152, a16+c153, a16+c154, a16+c155, a16+c156, a16+c157, a16+c158, a16+c159, a16+c160, a16+c161, a16+c162, a16+c163, a16+c164, a16+c165, a16+c166, a16+c167, a16+c168, a16+c169, a16+c170, a16+c171, a16+c172, a16+c173, a16+c174, a16+c175, a16+c176, a16+c177, a16+c178, a16+c179, a16+c180, a16+c181, a16+c182, a16+c183, a16+c184, a16+c185, a16+c186, a16+c187, a16+c188, a16+c189, a16+c190, a16+c191, a16+c192, a16+c193, a16+c194, a16+c195, a16+c196, a16+c197, a16+c198, a16+c199, a16+c200, a16+c201, a16+c202, a16+c203, a16+c204, a16+c205, a16+c206, a16+c207, a16+c208, a16+c209, a16+c210, a16+c211, a17+c1, a17+c2, a17+c3, a17+c4, a17+c5, a17+c6, a17+c7, a17+c8, a17+c9, a17+c10, a17+c11, a17+c12, a17+c13, a17+c14, a17+c15, a17+c16, a17+c17, a17+c18, a17+c19, a17+c20, a17+c21, a17+c22, a17+c23, a17+c24, a17+c25, a17+c26, a17+c27, a17+c28, a17+c29, a17+c30, a17+c31, a17+c32, a17+c33, a17+c34, a17+c35, a17+c36, a17+c37, a17+c38, a17+c39, a17+c40, a17+c41, a17+c42, a17+c43, a17+c44, a17+c45, a17+c46, a17+c47, a17+c48, a17+c49, a17+c50, a17+c51, a17+c52, a17+c53, a17+c54, a17+c55, a17+c56, a17+c57, a17+c58, a17+c59, a17+c60, a17+c61, a17+c62, a17+c63, a17+c64, a17+c65, a17+c66, a17+c67, a17+c68, a17+c69, a17+c70, a17+c71, a17+c72, a17+c73, a17+c74, a17+c75, a17+c76, a17+c77, a17+c78, a17+c79, a17+c80, a17+c81, a17+c82, a17+c83, a17+c84, a17+c85, a17+c86, a17+c87, a17+c88, a17+c89, a17+c90, a17+c91, a17+c92, a17+c93, a17+c94, a17+c95, a17+c96, a17+c97, a17+c98, a17+c99, a17+c100, a17+c101, a17+c102, a17+c103, a17+c104, a17+c105, a17+c106, a17+c107, a17+c108, a17+c109, a17+c110, a17+c111, a17+c112, a17+c113, a17+c114, a17+c115, a17+c116, a17+c117, a17+c118, a17+c119, a17+c120, a17+c121, a17+c122, a17+c123, a17+c124, a17+c125, a17+c126, a17+c127, a17+c128, a17+c129, a17+c130, a17+c131, a17+c132, a17+c133, a17+c134, a17+c135, a17+c136, a17+c137, a17+c138, a17+c139, a17+c140, a17+c141, a17+c142, a17+c143, a17+c144, a17+c145, a17+c146, a17+c147, a17+c148, a17+c149, a17+c150, a17+c151, a17+c152, a17+c153, a17+c154, a17+c155, a17+c156, a17+c157, a17+c158, a17+c159, a17+c160, a17+c161, a17+c162, a17+c163, a17+c164, a17+c165, a17+c166, a17+c167, a17+c168, a17+c169, a17+c170, a17+c171, a17+c172, a17+c173, a17+c174, a17+c175, a17+c176, a17+c177, a17+c178, a17+c179, a17+c180, a17+c181, a17+c182, a11+c183, a11+c184, a17+c185, a17+c186, a17+c187, a17+c188, a17+c189, a17+c190, a17+c191, a17+c192, a17+c193, a17+c194, a17+c195, a17+c196, a17+c197, a17+c198, a17+c199, a17+c200, a17+c201, a17+c202, a17+c203, a17+c204, a17+c205, a17+c206, a17+c207, a17+c208, a17+c209, a17+c210, a17+c211, a18+c1, a18+c2, a18+c3, a18+c4, a18+c5, a18+c6, a18+c7, a18+c8, a18+c9, a18+c10, a18+c11, a18+c12, a18+c13, a18+c14, a18+c15, a18+c16, a18+c17, a18+c18, a18+c19, a18+c20, a18+c21, a18+c22, a18+c23, a18+c24, a18+c25, a18+c26, a18+c27, a18+c28, a18+c29, a18+c30, a18+c31, a18+c32, a18+c33, a18+c34, a18+c35, a18+c36, a18+c37, a18+c38, a18+c39, a18+c40, a18+c41, a18+c42, a18+c43, a18+c44, a18+c45, a18+c46, a18+c47, a18+c48, a18+c49, a18+c50, a18+c51, a18+c52, a18+c53, a18+c54, a18+c55, a18+c56, a18+c57, a18+c58, a18+c59, a18+c60, a18+c61, a18+c62, a18+c63, a18+c64, a18+c65, a18+c66, a18+c67, a18+c68, a18+c69, a18+c70, a18+c71, a18+c72, a18+c73, a18+c74, a18+c75, a18+c76, a18+c77, a18+c78, a18+c79, a18+c80, a18+c81, a18+c82, a18+c83, a18+c84, a18+c85, a18+c86, a18+c87, a18+c88, a18+c89, a18+c90, a18+c91, a18+c92, a18+c93, a18+c94, a18+c95, a18+c96, a18+c97, a18+c98, a18+c99, a18+c100, a18+c101, a18+c102, a18+c103, a18+c104, a18+c105, a18+c106, a18+c107, a18+c108, a18+c109, a18+c110, a18+c111, a18+c112, a18+c113, a18+c114, a18+c115, a18+c116, a18+c117, a18+c118, a18+c119, a18+c120, a18+c121, a18+c122, a18+c123, a18+c124, a18+c125, a18+c126, a18+c127, a18+c128, a18+c129, a18+c130, a18+c131, a18+c132, a18+c133, a18+c134, a18+c135, a18+c136, a18+c137, a18+c138, a18+c139, a18+c140, a18+c141, a18+c142, a18+c143, a18+c144, a18+c145, a18+c146, a18+c147, a18+c148, a18+c149, a18+c150, a18+c151, a18+c152, a18+c153, a18+c154, a18+c155, a18+c156, a18+c157, a18+c158, a18+c159, a18+c160, a18+c161, a18+c162, a18+c163, a18+c164, a18+c165, a18+c166, a18+c167, a18+c168, a18+c169, a18+c170, a18+c171, a18+c172, a18+c173, a18+c174, a18+c175, a18+c176, a18+c177, a18+c178, a18+c179, a18+c180, a18+c181, a18+c182, a18+c183, a18+c184, a18+c185, a18+c186, a18+c187, a18+c188, a18+c189, a18+c190, a18+c191, a18+c192, a18+c193, a18+c194, a18+c195, a18+c196, a18+c197, a18+c198, a18+c199, a18+c200, a18+c201, a18+c202, a18+c203, a18+c204, a18+c205, a18+c206, a18+c207, a18+c208, a18+c209, a18+c210, a18+c211, a19+c1, a19+c2, a19+c3, a19+c4, a19+c5, a19+c6, a19+c7, a19+c8, a19+c9, a19+c10, a19+c11, a19+c12, a19+c13, a19+c14, a19+c15, a19+c16, a19+c17, a19+c18, a19+c19, a19+c20, a19+c21, a19+c22, a19+c23, a19+c24, a19+c25, a19+c26, a19+c27, a19+c28, a19+c29, a19+c30, a19+c31, a19+c32, a19+c33, a19+c34, a19+c35, a19+c36, a19+c37, a19+c38, a19+c39, a19+c40, a19+c41, a19+c42, a19+c43, a19+c44, a19+c45, a19+c46, a19+c47, a19+c48, a19+c49, a19+c50, a19+c51, a19+c52, a19+c53, a19+c54, a19+c55, a19+c56, a19+c57, a19+c58, a19+c59, a19+c60, a19+c61, a19+c62, a19+c63, a19+c64, a19+c65, a19+c66, a19+c67, a19+c68, a19+c69, a19+c70, a19+c71, a19+c72, a19+c73, a19+c74, a19+c75, a19+c76, a19+c77, a19+c78, a19+c79, a19+c80, a19+c81, a19+c82, a19+c83, a19+c84, a19+c85, a19+c86, a19+c87, a19+c88, a19+c89, a19+c90, a19+c91, a19+c92, a19+c93, a19+c94, a19+c95, a19+c96, a19+c97, a19+c98, a19+c99, a19+c100, a19+c101, a19+c102, a19+c103, a19+c104, a19+c105, a19+c106, a19+c107, a19+c108, a19+c109, a19+c110, a19+c111, a19+c112, a19+c113, a19+c114, a19+c115, a19+c116, a19+c117, a19+c118, a19+c119, a19+c120, a19+c121, a19+c122, a19+c123, a19+c124, a19+c125, a19+c126, a19+c127, a19+c128, a19+c129, a19+c130, a19+c131, a19+c132, a19+c133, a19+c134, a19+c135, a19+c136, a19+c137, a19+c138, a19+c139, a19+c140, a19+c141, a19+c142, a19+c143, a19+c144, a19+c145, a19+c146, a19+c147, a19+c148, a19+c149, a19+c150, a19+c151, a19+c152, a19+c153, a19+c154, a19+c155, a19+c156, a19+c157, a19+c158, a19+c159, a19+c160, a19+c161, a19+c162, a19+c163, a19+c164, a19+c165, a19+c166, a19+c167, a19+c168, a19+c169, a19+c170, a19+c171, a19+c172, a19+c173, a19+c174, a19+c175, a19+c176, a19+c177, a19+c178, a19+c179, a19+c180, a19+c181, a19+c182, a19+c183, a19+c184, a19+c185, a19+c186, a19+c187, a19+c188, a19+c189, a19+c190, a19+c191, a19+c192, a19+c193, a19+c194, a19+c195, a19+c196, a19+c197, a19+c198, a19+c199, a19+c200, a19+c201, a19+c202, a19+c203, a19+c204, a19+c205, a19+c206, a19+c207, a19+c208, a19+c209, a19+c210, a19+c211, a20+c1, a20+c2, a20+c3, a20+c4, a20+c5, a20+c6, a20+c7, a20+c8, a20+c9, a20+c10, a20+c11, a20+c12, a20+c13, a20+c14, a20+c15, a20+c16, a20+c17, a20+c18, a20+c19, a20+c20, a20+c21, a20+c22, a20+c23, a20+c24, a20+c25, a20+c26, a20+c27, a20+c28, a20+c29, a20+c30, a20+c31, a20+c32, a20+c33, a20+c34, a20+c35, a20+c36, a20+c37, a20+c38, a20+c39, a20+c40, a20+c41, a20+c42, a20+c43, a20+c44, a20+c45, a20+c46, a20+c47, a20+c48, a20+c49, a20+c50, a20+c51, a20+c52, a20+c53, a20+c54, a20+c55, a20+c56, a20+c57, a20+c58, a20+c59, a20+c60, a20+c61, a20+c62, a20+c63, a20+c64, a20+c65, a20+c66, a20+c67, a20+c68, a20+c69, a20+c70, a20+c71, a20+c72, a20+c73, a20+c74, a20+c75, a20+c76, a20+c77, a20+c78, a20+c79, a20+c80, a20+c81, a20+c82, a20+c83, a20+c84, a20+c85, a20+c86, a20+c87, a20+c88, a20+c89, a20+c90, a20+c91, a20+c92, a20+c93, a20+c94, a20+c95, a20+c96, a20+c97, a20+c98, a20+c99, a20+c100, a20+c101, a20+c102, a20+c103, a20+c104, a20+c105, a20+c106, a20+c107, a20+c108, a20+c109, a20+c110, a20+c111, a20+c112, a20+c113, a20+c114, a20+c115, a20+c116, a20+c117, a20+c118, a20+c119, a20+c120, a20+c121, a20+c122, a20+c123, a20+c124, a20+c125, a20+c126, a20+c127, a20+c128, a20+c129, a20+c130, a20+c131, a20+c132, a20+c133, a20+c134, a20+c135, a20+c136, a20+c137, a20+c138, a20+c139, a20+c140, a20+c141, a20+c142, a20+c143, a20+c144, a20+c145, a20+c146, a20+c147, a20+c148, a20+c149, a20+c150, a20+c151, a20+c152, a20+c153, a20+c154, a20+c155, a20+c156, a20+c157, a20+c158, a20+c159, a20+c160, a20+c161, a20+c162, a20+c163, a20+c164, a20+c165, a20+c166, a20+c167, a20+c168, a20+c169, a20+c170, a20+c171, a20+c172, a20+c173, a20+c174, a20+c175, a20+c176, a20+c177, a20+c178, a20+c179, a20+c180, a20+c181, a20+c182, a20+c183, a20+c184, a20+c185, a20+c186, a20+c187, a20+c188, a20+c189, a20+c190, a20+c191, a20+c192, a20+c193, a20+c194, a20+c195, a20+c196, a20+c197, a20+c198, a20+c199, a20+c200, a20+c201, a20+c202, a20+c203, a20+c204, a20+c205, a20+c206, a20+c207, a20+c208, a20+c209, a20+c210, a20+c211, a21+c1, a21+c2, a21+c3, a21+c4, a21+c5, a21+c6, a21+c7, a21+c8, a21+c9, a21+c10, a21+c11, a21+c12, a21+c13, a21+c14, a21+c15, a21+c16, a21+c17, a21+c18, a21+c19, a21+c20, a21+c21, a21+c22, a21+c23, a21+c24, a21+c25, a21+c26, a21+c27, a21+c28, a21+c29, a21+c30, a21+c31, a21+c32, a21+c33, a21+c34, a21+c35, a21+c36, a21+c37, a21+c38, a21+c39, a21+c40, a21+c41, a21+c42, a21+c43, a21+c44, a21+c45, a21+c46, a21+c47, a21+c48, a21+c49, a21+c50, a21+c51, a21+c52, a21+c53, a21+c54, a21+c55, a21+c56, a21+c57, a21+c58, a21+c59, a21+c60, a21+c61, a21+c62, a21+c63, a21+c64, a21+c65, a21+c66, a21+c67, a21+c68, a21+c69, a21+c70, a21+c71, a21+c72, a21+c73, a21+c74, a21+c75, a21+c76, a21+c77, a21+c78, a21+c79, a21+c80, a21+c81, a21+c82, a21+c83, a21+c84, a21+c85, a21+c86, a21+c87, a21+c88, a21+c89, a21+c90, a21+c91, a21+c92, a21+c93, a21+c94, a21+c95, a21+c96, a21+c97, a21+c98, a21+c99, a21+c100, a21+c101, a21+c102, a21+c103, a21+c104, a21+c105, a21+c106, a21+c107, a21+c108, a21+c109, a21+c110, a21+c111, a21+c112, a21+c113, a21+c114, a21+c115, a21+c116, a21+c117, a21+c118, a21+c119, a21+c120, a21+c121, a21+c122, a21+c123, a21+c124, a21+c125, a21+c126, a21+c127, a21+c128, a21+c129, a21+c130, a21+c131, a21+c132, a21+c133, a21+c134, a21+c135, a21+c136, a21+c137, a21+c138, a21+c139, a21+c140, a21+c141, a21+c142, a21+c143, a21+c144, a21+c145, a21+c146, a21+c147, a21+c148, a21+c149, a21+c150, a21+c151, a21+c152, a21+c153, a21+c154, a21+c155, a21+c156, a21+c157, a21+c158, a21+c159, a21+c160, a21+c161, a21+c162, a21+c163, a21+c164, a21+c165, a21+c166, a21+c167, a21+c168, a21+c169, a21+c170, a21+c171, a21+c172, a21+c173, a21+c174, a21+c175, a21+c176, a21+c177, a21+c178, a21+c179, a21+c180, a21+c181, a21+c182, a21+c183, a21+c184, a21+c185, a21+c186, a21+c187, a21+c188, a21+c189, a21+c190, a21+c191, a21+c192, a21+c193, a21+c194, a21+c195, a21+c196, a21+c197, a21+c198, a21+c199, a21+c200, a21+c201, a21+c202, a21+c203, a21+c204, a21+c205, a21+c206, a21+c207, a21+c208, a21+c209, a21+c210, a21+c211, a22+c1, a22+c2, a22+c3, a22+c4, a22+c5, a22+c6, a22+c7, a22+c8, a22+c9, a22+c10, a22+c11, a22+c12, a22+c13, a22+c14, a22+c15, a22+c16, a22+c17, a22+c18, a22+c19, a22+c20, a22+c21, a22+c22, a22+c23, a22+c24, a22+c25, a22+c26, a22+c27, a22+c28, a22+c29, a22+c30, a22+c31, a22+c32, a22+c33, a22+c34, a22+c35, a22+c36, a22+c37, a22+c38, a22+c39, a22+c40, a22+c41, a22+c42, a22+c43, a22+c44, a22+c45, a22+c46, a22+c47, a22+c48, a22+c49, a22+c50, a22+c51, a22+c52, a22+c53, a22+c54, a22+c55, a22+c56, a22+c57, a22+c58, a22+c59, a22+c60, a22+c61, a22+c62, a22+c63, a22+c64, a22+c65, a22+c66, a22+c67, a22+c68, a22+c69, a22+c70, a22+c71, a22+c72, a22+c73, a22+c74, a22+c75, a22+c76, a22+c77, a22+c78, a22+c79, a22+c80, a22+c81, a22+c82, a22+c83, a22+c84, a22+c85, a22+c86, a22+c87, a22+c88, a22+c89, a22+c90, a22+c91, a22+c92, a22+c93, a22+c94, a22+c95, a22+c96, a22+c97, a22+c98, a22+c99, a22+c100, a22+c101, a22+c102, a22+c103, a22+c104, a22+c105, a22+c106, a22+c107, a22+c108, a22+c109, a22+c110, a22+c111, a22+c112, a22+c113, a22+c114, a22+c115, a22+c116, a22+c117, a22+c118, a22+c119, a22+c120, a22+c121, a22+c122, a22+c123, a22+c124, a22+c125, a22+c126, a22+c127, a22+c128, a22+c129, a22+c130, a22+c131, a22+c132, a22+c133, a22+c134, a22+c135, a22+c136, a22+c137, a22+c138, a22+c139, a22+c140, a22+c141, a22+c142, a22+c143, a22+c144, a22+c145, a22+c146, a22+c147, a22+c148, a22+c149, a22+c150, a22+c151, a22+c152, a22+c153, a22+c154, a22+c155, a22+c156, a22+c157, a22+c158, a22+c159, a22+c160, a22+c161, a22+c162, a22+c163, a22+c164, a22+c165, a22+c166, a22+c167, a22+c168, a22+c169, a22+c170, a22+c171, a22+c172, a22+c173, a22+c174, a22+c175, a22+c176, a22+c177, a22+c178, a22+c179, a22+c180, a22+c181, a22+c182, a22+c183, a22+c184, a22+c185, a22+c186, a22+c187, a22+c188, a22+c189, a22+c190, a22+c191, a22+c192, a22+c193, a22+c194, a22+c195, a22+c196, a22+c197, a22+c198, a22+c199, a22+c200, a22+c201, a22+c202, a22+c203, a22+c204, a22+c205, a22+c206, a22+c207, a22+c208, a22+c209, a22+c210, a22+c211, a23+c1, a23+c2, a23+c3, a23+c4, a23+c5, a23+c6, a23+c7, a23+c8, a23+c9, a23+c10, a23+c11, a23+c12, a23+c13, a23+c14, a23+c15, a23+c16, a23+c17, a23+c18, a23+c19, a23+c20, a23+c21, a23+c22, a23+c23, a23+c24, a23+c25, a23+c26, a23+c27, a23+c28, a23+c29, a23+c30, a23+c31, a23+c32, a23+c33, a23+c34, a23+c35, a23+c36, a23+c37, a23+c38, a23+c39, a23+c40, a23+c41, a23+c42, a23+c43, a23+c44, a23+c45, a23+c46, a23+c47, a23+c48, a23+c49, a23+c50, a23+c51, a23+c52, a23+c53, a23+c54, a23+c55, a23+c56, a23+c57, a23+c58, a23+c59, a23+c60, a23+c61, a23+c62, a23+c63, a23+c64, a23+c65, a23+c66, a23+c67, a23+c68, a23+c69, a23+c70, a23+c71, a23+c72, a23+c73, a23+c74, a23+c75, a23+c76, a23+c77, a23+c78, a23+c79, a23+c80, a23+c81, a23+c82, a23+c83, a23+c84, a23+c85, a23+c86, a23+c87, a23+c88, a23+c89, a23+c90, a23+c91, a23+c92, a23+c93, a23+c94, a23+c95, a23+c96, a23+c97, a23+c98, a23+c99, a23+c100, a23+c101, a23+c102, a23+c103, a23+c104, a23+c105, a23+c106, a23+c107, a23+c108, a23+c109, a23+c110, a23+c111, a23+c112, a23+c113, a23+c114, a23+c115, a23+c116, a23+c117, a23+c118, a23+c119, a23+c120, a23+c121, a23+c122, a23+c123, a23+c124, a23+c125, a23+c126, a23+c127, a23+c128, a23+c129, a23+c130, a23+c131, a23+c132, a23+c133, a23+c134, a23+c135, a23+c136, a23+c137, a23+c138, a23+c139, a23+c140, a23+c141, a23+c142, a23+c143, a23+c144, a23+c145, a23+c146, a23+c147, a23+c148, a23+c149, a23+c150, a23+c151, a23+c152, a23+c153, a23+c154, a23+c155, a23+c156, a23+c157, a23+c158, a23+c159, a23+c160, a23+c161, a23+c162, a23+c163, a23+c164, a23+c165, a23+c166, a23+c167, a23+c168, a23+c169, a23+c170, a23+c171, a23+c172, a23+c173, a23+c174, a23+c175, a23+c176, a23+c177, a23+c178, a23+c179, a23+c180, a23+c181, a23+c182, a23+c183, a23+c184, a23+c185, a23+c186, a23+c187, a23+c188, a23+c189, a23+c190, a23+c191, a23+c192, a23+c193, a23+c194, a23+c195, a23+c196, a23+c197, a23+c198, a23+c199, a23+c200, a23+c201, a23+c202, a23+c203, a23+c204, a23+c205, a23+c206, a23+c207, a23+c208, a23+c209, a23+c210, a23+c211, a24+c1, a24+c2, a24+c3, a24+c4, a24+c5, a24+c6, a24+c7, a24+c8, a24+c9, a24+c10, a24+c11, a24+c12, a24+c13, a24+c14, a24+c15, a24+c16, a24+c17, a24+c18, a24+c19, a24+c20, a24+c21, a24+c22, a24+c23, a24+c24, a24+c25, a24+c26, a24+c27, a24+c28, a24+c29, a24+c30, a24+c31, a24+c32, a24+c33, a24+c34, a24+c35, a24+c36, a24+c37, a24+c38, a24+c39, a24+c40, a24+c41, a24+c42, a24+c43, a24+c44, a24+c45, a24+c46, a24+c47, a24+c48, a24+c49, a24+c50, a24+c51, a24+c52, a24+c53, a24+c54, a24+c55, a24+c56, a24+c57, a24+c58, a24+c59, a24+c60, a24+c61, a24+c62, a24+c63, a24+c64, a24+c65, a24+c66, a24+c67, a24+c68, a24+c69, a24+c70, a24+c71, a24+c72, a24+c73, a24+c74, a24+c75, a24+c76, a24+c77, a24+c78, a24+c79, a24+c80, a24+c81, a24+c82, a24+c83, a24+c84, a24+c85, a24+c86, a24+c87, a24+c88, a24+c89, a24+c90, a24+c91, a24+c92, a24+c93, a24+c94, a24+c95, a24+c96, a24+c97, a24+c98, a24+c99, a24+c100, a24+c101, a24+c102, a24+c103, a24+c104, a24+c105, a24+c106, a24+c107, a24+c108, a24+c109, a24+c110, a24+c111, a24+c112, a24+c113, a24+c114, a24+c115, a24+c116, a24+c117, a24+c118, a24+c119, a24+c120, a24+c121, a24+c122, a24+c123, a24+c124, a24+c125, a24+c126, a24+c127, a24+c128, a24+c129, a24+c130, a24+c131, a24+c132, a24+c133, a24+c134, a24+c135, a24+c136, a24+c137, a24+c138, a24+c139, a24+c140, a24+c141, a24+c142, a24+c143, a24+c144, a24+c145, a24+c146, a24+c147, a24+c148, a24+c149, a24+c150, a24+c151, a24+c152, a24+c153, a24+c154, a24+c155, a24+c156, a24+c157, a24+c158, a24+c159, a24+c160, a24+c161, a24+c162, a24+c163, a24+c164, a24+c165, a24+c166, a24+c167, a24+c168, a24+c169, a24+c170, a24+c171, a24+c172, a24+c173, a24+c174, a24+c175, a24+c176, a24+c177, a24+c178, a24+c179, a24+c180, a24+c181, a24+c182, a24+c183, a24+c184, a24+c185, a24+c186, a24+c187, a24+c188, a24+c189, a24+c190, a24+c191, a24+c192, a24+c193, a24+c194, a24+c195, a24+c196, a24+c197, a24+c198, a24+c199, a24+c200, a24+c201, a24+c202, a24+c203, a24+c204, a24+c205, a24+c206, a24+c207, a24+c208, a24+c209, a24+c210, a24+c211, a25+c1, a25+c2, a25+c3, a25+c4, a25+c5, a25+c6, a25+c7, a25+c8, a25+c9, a25+c10, a25+c11, a25+c12, a25+c13, a25+c14, a25+c15, a25+c16, a25+c17, a25+c18, a25+c19, a25+c20, a25+c21, a25+c22, a25+c23, a25+c24, a25+c25, a25+c26, a25+c27, a25+c28, a25+c29, a25+c30, a25+c31, a25+c32, a25+c33, a25+c34, a25+c35, a25+c36, a25+c37, a25+c38, a25+c39, a25+c40, a25+c41, a25+c42, a25+c43, a25+c44, a25+c45, a25+c46, a25+c47, a25+c48, a25+c49, a25+c50, a25+c51, a25+c52, a25+c53, a25+c54, a25+c55, a25+c56, a25+c57, a25+c58, a25+c59, a25+c60, a25+c61, a25+c62, a25+c63, a25+c64, a25+c65, a25+c66, a25+c67, a25+c68, a25+c69, a25+c70, a25+c71, a25+c72, a25+c73, a25+c74, a25+c75, a25+c76, a25+c77, a25+c78, a25+c79, a25+c80, a25+c81, a25+c82, a25+c83, a25+c84, a25+c85, a25+c86, a25+c87, a25+c88, a25+c89, a25+c90, a25+c91, a25+c92, a25+c93, a25+c94, a25+c95, a25+c96, a25+c97, a25+c98, a25+c99, a25+c100, a25+c101, a25+c102, a25+c103, a25+c104, a25+c105, a25+c106, a25+c107, a25+c108, a25+c109, a25+c110, a25+c111, a25+c112, a25+c113, a25+c114, a25+c115, a25+c116, a25+c117, a25+c118, a25+c119, a25+c120, a25+c121, a25+c122, a25+c123, a25+c124, a25+c125, a25+c126, a25+c127, a25+c128, a25+c129, a25+c130, a25+c131, a25+c132, a25+c133, a25+c134, a25+c135, a25+c136, a25+c137, a25+c138, a25+c139, a25+c140, a25+c141, a25+c142, a25+c143, a25+c144, a25+c145, a25+c146, a25+c147, a25+c148, a25+c149, a25+c150, a25+c151, a25+c152, a25+c153, a25+c154, a25+c155, a25+c156, a25+c157, a25+c158, a25+c159, a25+c160, a25+c161, a25+c162, a25+c163, a25+c164, a25+c165, a25+c166, a25+c167, a25+c168, a25+c169, a25+c170, a25+c171, a25+c172, a25+c173, a25+c174, a25+c175, a25+c176, a25+c177, a25+c178, a25+c179, a25+c180, a25+c181, a25+c182, a25+c183, a25+c184, a25+c185, a25+c186, a25+c187, a25+c188, a25+c189, a25+c190, a25+c191, a25+c192, a25+c193, a25+c194, a25+c195, a25+c196, a25+c197, a25+c198, a25+c199, a25+c200, a25+c201, a25+c202, a25+c203, a25+c204, a25+c205, a25+c206, a25+c207, a25+c208, a25+c209, a25+c210, a25+c211, a26+c1, a26+c2, a26+c3, a26+c4, a26+c5, a26+c6, a26+c7, a26+c8, a26+c9, a26+c10, a26+c11, a26+c12, a26+c13, a26+c14, a26+c15, a26+c16, a26+c17, a26+c18, a26+c19, a26+c20, a26+c21, a26+c22, a26+c23, a26+c24, a26+c25, a26+c26, a26+c27, a26+c28, a26+c29, a26+c30, a26+c31, a26+c32, a26+c33, a26+c34, a26+c35, a26+c36, a26+c37, a26+c38, a26+c39, a26+c40, a26+c41, a26+c42, a26+c43, a26+c44, a26+c45, a26+c46, a26+c47, a26+c48, a26+c49, a26+c50, a26+c51, a26+c52, a26+c53, a26+c54, a26+c55, a26+c56, a26+c57, a26+c58, a26+c59, a26+c60, a26+c61, a26+c62, a26+c63, a26+c64, a26+c65, a26+c66, a26+c67, a26+c68, a26+c69, a26+c70, a26+c71, a26+c72, a26+c73, a26+c74, a26+c75, a26+c76, a26+c77, a26+c78, a26+c79, a26+c80, a26+c81, a26+c82, a26+c83, a26+c84, a26+c85, a26+c86, a26+c87, a26+c88, a26+c89, a26+c90, a26+c91, a26+c92, a26+c93, a26+c94, a26+c95, a26+c96, a26+c97, a26+c98, a26+c99, a26+c100, a26+c101, a26+c102, a26+c103, a26+c104, a26+c105, a26+c106, a26+c107, a26+c108, a26+c109, a26+c110, a26+c111, a26+c112, a26+c113, a26+c114, a26+c115, a26+c116, a26+c117, a26+c118, a26+c119, a26+c120, a26+c121, a26+c122, a26+c123, a26+c124, a26+c125, a26+c126, a26+c127, a26+c128, a26+c129, a26+c130, a26+c131, a26+c132, a26+c133, a26+c134, a26+c135, a26+c136, a26+c137, a26+c138, a26+c139, a26+c140, a26+c141, a26+c142, a26+c143, a26+c144, a26+c145, a26+c146, a26+c147, a26+c148, a26+c149, a26+c150, a26+c151, a26+c152, a26+c153, a26+c154, a26+c155, a26+c156, a26+c157, a26+c158, a26+c159, a26+c160, a26+c161, a26+c162, a26+c163, a26+c164, a26+c165, a26+c166, a26+c167, a26+c168, a26+c169, a26+c170, a26+c171, a26+c172, a26+c173, a26+c174, a26+c175, a26+c176, a26+c177, a26+c178, a26+c179, a26+c180, a26+c181, a26+c182, a26+c183, a26+c184, a26+c185, a26+c186, a26+c187, a26+c188, a26+c189, a26+c190, a26+c191, a26+c192, a26+c193, a26+c194, a26+c195, a26+c196, a26+c197, a26+c198, a26+c199, a26+c200, a26+c201, a26+c202, a26+c203, a26+c204, a26+c205, a26+c206, a26+c207, a26+c208, a26+c209, a26+c210, a26+c211, a27+c1, a27+c2, a27+c3, a27+c4, a27+c5, a27+c6, a27+c7, a27+c8, a27+c9, a27+c10, a27+c11, a27+c12, a27+c13, a27+c14, a27+c15, a27+c16, a27+c17, a27+c18, a27+c19, a27+c20, a27+c21, a27+c22, a27+c23, a27+c24, a27+c25, a27+c26, a27+c27, a27+c28, a27+c29, a27+c30, a27+c31, a27+c32, a27+c33, a27+c34, a27+c35, a27+c36, a27+c37, a27+c38, a27+c39, a27+c40, a27+c41, a27+c42, a27+c43, a27+c44, a27+c45, a27+c46, a27+c47, a27+c48, a27+c49, a27+c50, a27+c51, a27+c52, a27+c53, a27+c54, a27+c55, a27+c56, a27+c57, a27+c58, a27+c59, a27+c60, a27+c61, a27+c62, a27+c63, a27+c64, a27+c65, a27+c66, a27+c67, a27+c68, a27+c69, a27+c70, a27+c71, a27+c72, a27+c73, a27+c74, a27+c75, a27+c76, a27+c77, a27+c78, a27+c79, a27+c80, a27+c81, a27+c82, a27+c83, a27+c84, a27+c85, a27+c86, a27+c87, a27+c88, a27+c89, a27+c90, a27+c91, a27+c92, a27+c93, a27+c94, a27+c95, a27+c96, a27+c97, a27+c98, a27+c99, a27+c100, a27+c101, a27+c102, a27+c103, a27+c104, a27+c105, a27+c106, a27+c107, a27+c108, a27+c109, a27+c110, a27+c111, a27+c112, a27+c113, a27+c114, a27+c115, a27+c116, a27+c117, a27+c118, a27+c119, a27+c120, a27+c121, a27+c122, a27+c123, a27+c124, a27+c125, a27+c126, a27+c127, a27+c128, a27+c129, a27+c130, a27+c131, a27+c132, a27+c133, a27+c134, a27+c135, a27+c136, a27+c137, a27+c138, a27+c139, a27+c140, a27+c141, a27+c142, a27+c143, a27+c144, a27+c145, a27+c146, a27+c147, a27+c148, a27+c149, a27+c150, a27+c151, a27+c152, a27+c153, a27+c154, a27+c155, a27+c156, a27+c157, a27+c158, a27+c159, a27+c160, a27+c161, a27+c162, a27+c163, a27+c164, a27+c165, a27+c166, a27+c167, a27+c168, a27+c169, a27+c170, a27+c171, a27+c172, a27+c173, a27+c174, a27+c175, a27+c176, a27+c177, a27+c178, a27+c179, a27+c180, a27+c181, a27+c182, a27+c183, a27+c184, a27+c185, a27+c186, a27+c187, a27+c188, a27+c189, a27+c190, a27+c191, a27+c192, a27+c193, a27+c194, a27+c195, a27+c196, a27+c197, a27+c198, a27+c199, a27+c200, a27+c201, a27+c202, a27+c203, a27+c204, a27+c205, a27+c206, a27+c207, a27+c208, a27+c209, a27+c210, a27+c211, a28+c1, a28+c2, a28+c3, a28+c4, a28+c5, a28+c6, a28+c7, a28+c8, a28+c9, a28+c10, a28+c11, a28+c12, a28+c13, a28+c14, a28+c15, a28+c16, a28+c17, a28+c18, a28+c19, a28+c20, a28+c21, a28+c22, a28+c23, a28+c24, a28+c25, a28+c26, a28+c27, a28+c28, a28+c29, a28+c30, a28+c31, a28+c32, a28+c33, a28+c34, a28+c35, a28+c36, a28+c37, a28+c38, a28+c39, a28+c40, a28+c41, a28+c42, a28+c43, a28+c44, a28+c45, a28+c46, a28+c47, a28+c48, a28+c49, a28+c50, a28+c51, a28+c52, a28+c53, a28+c54, a28+c55, a28+c56, a28+c57, a28+c58, a28+c59, a28+c60, a28+c61, a28+c62, a28+c63, a28+c64, a28+c65, a28+c66, a28+c67, a28+c68, a28+c69, a28+c70, a28+c71, a28+c72, a28+c73, a28+c74, a28+c75, a28+c76, a28+c77, a28+c78, a28+c79, a28+c80, a28+c81, a28+c82, a28+c83, a28+c84, a28+c85, a28+c86, a28+c87, a28+c88, a28+c89, a28+c90, a28+c91, a28+c92, a28+c93, a28+c94, a28+c95, a28+c96, a28+c97, a28+c98, a28+c99, a28+c100, a28+c101, a28+c102, a28+c103, a28+c104, a28+c105, a28+c106, a28+c107, a28+c108, a28+c109, a28+c110, a28+c111, a28+c112, a28+c113, a28+c114, a28+c115, a28+c116, a28+c117, a28+c118, a28+c119, a28+c120, a28+c121, a28+c122, a28+c123, a28+c124, a28+c125, a28+c126, a28+c127, a28+c128, a28+c129, a28+c130, a28+c131, a28+c132, a28+c133, a28+c134, a28+c135, a28+c136, a28+c137, a28+c138, a28+c139, a28+c140, a28+c141, a28+c142, a28+c143, a28+c144, a28+c145, a28+c146, a28+c147, a28+c148, a28+c149, a28+c150, a28+c151, a28+c152, a28+c153, a28+c154, a28+c155, a28+c156, a28+c157, a28+c158, a28+c159, a28+c160, a28+c161, a28+c162, a28+c163, a28+c164, a28+c165, a28+c166, a28+c167, a28+c168, a28+c169, a28+c170, a28+c171, a28+c172, a28+c173, a28+c174, a28+c175, a28+c176, a28+c177, a28+c178, a28+c179, a28+c180, a28+c181, a28+c182, a28+c183, a28+c184, a28+c185, a28+c186, a28+c187, a28+c188, a28+c189, a28+c190, a28+c191, a28+c192, a28+c193, a28+c194, a28+c195, a28+c196, a28+c197, a28+c198, a28+c199, a28+c200, a28+c201, a28+c202, a28+c203, a28+c204, a28+c205, a28+c206, a28+c207, a28+c208, a28+c209, a28+c210, a28+c211, a29+c1, a29+c2, a29+c3, a29+c4, a29+c5, a29+c6, a29+c7, a29+c8, a29+c9, a29+c10, a29+c11, a29+c12, a29+c13, a29+c14, a29+c15, a29+c16, a29+c17, a29+c18, a29+c19, a29+c20, a29+c21, a29+c22, a29+c23, a29+c24, a29+c25, a29+c26, a29+c27, a29+c28, a29+c29, a29+c30, a29+c31, a29+c32, a29+c33, a29+c34, a29+c35, a29+c36, a29+c37, a29+c38, a29+c39, a29+c40, a29+c41, a29+c42, a29+c43, a29+c44, a29+c45, a29+c46, a29+c47, a29+c48, a29+c49, a29+c50, a29+c51, a29+c52, a29+c53, a29+c54, a29+c55, a29+c56, a29+c57, a29+c58, a29+c59, a29+c60, a29+c61, a29+c62, a29+c63, a29+c64, a29+c65, a29+c66, a29+c67, a29+c68, a29+c69, a29+c70, a29+c71, a29+c72, a29+c73, a29+c74, a29+c75, a29+c76, a29+c77, a29+c78, a29+c79, a29+c80, a29+c81, a29+c82, a29+c83, a29+c84, a29+c85, a29+c86, a29+c87, a29+c88, a29+c89, a29+c90, a29+c91, a29+c92, a29+c93, a29+c94, a29+c95, a29+c96, a29+c97, a29+c98, a29+c99, a29+c100, a29+c101, a29+c102, a29+c103, a29+c104, a29+c105, a29+c106, a29+c107, a29+c108, a29+c109, a29+c110, a29+c111, a29+c112, a29+c113, a29+c114, a29+c115, a29+c116, a29+c117, a29+c118, a29+c119, a29+c120, a29+c121, a29+c122, a29+c123, a29+c124, a29+c125, a29+c126, a29+c127, a29+c128, a29+c129, a29+c130, a29+c131, a29+c132, a29+c133, a29+c134, a29+c135, a29+c136, a29+c137, a29+c138, a29+c139, a29+c140, a29+c141, a29+c142, a29+c143, a29+c144, a29+c145, a29+c146, a29+c147, a29+c148, a29+c149, a29+c150, a29+c151, a29+c152, a29+c153, a29+c154, a29+c155, a29+c156, a29+c157, a29+c158, a29+c159, a29+c160, a29+c161, a29+c162, a29+c163, a29+c164, a29+c165, a29+c166, a29+c167, a29+c168, a29+c169, a29+c170, a29+c171, a29+c172, a29+c173, a29+c174, a29+c175, a29+c176, a29+c177, a29+c178, a29+c179, a29+c180, a29+c181, a29+c182, a29+c183, a29+c184, a29+c185, a29+c186, a29+c187, a29+c188, a29+c189, a29+c190, a29+c191, a29+c192, a29+c193, a29+c194, a29+c195, a29+c196, a29+c197, a29+c198, a29+c199, a29+c200, a29+c201, a29+c202, a29+c203, a29+c204, a29+c205, a29+c206, a29+c207, a29+c208, a29+c209, a29+c210, a29+c211, a30+c1, a30+c2, a30+c3, a30+c4, a30+c5, a30+c6, a30+c7, a30+c8, a30+c9, a30+c10, a30+c11, a30+c12, a30+c13, a30+c14, a30+c15, a30+c16, a30+c17, a30+c18, a30+c19, a30+c20, a30+c21, a30+c22, a30+c23, a30+c24, a30+c25, a30+c26, a30+c27, a30+c28, a30+c29, a30+c30, a30+c31, a30+c32, a30+c33, a30+c34, a30+c35, a30+c36, a30+c37, a30+c38, a30+c39, a30+c40, a30+c41, a30+c42, a30+c43, a30+c44, a30+c45, a30+c46, a30+c47, a30+c48, a30+c49, a30+c50, a30+c51, a30+c52, a30+c53, a30+c54, a30+c55, a30+c56, a30+c57, a30+c58, a30+c59, a30+c60, a30+c61, a30+c62, a30+c63, a30+c64, a30+c65, a30+c66, a30+c67, a30+c68, a30+c69, a30+c70, a30+c71, a30+c72, a30+c73, a30+c74, a30+c75, a30+c76, a30+c77, a30+c78, a30+c79, a30+c80, a30+c81, a30+c82, a30+c83, a30+c84, a30+c85, a30+c86, a30+c87, a30+c88, a30+c89, a30+c90, a30+c91, a30+c92, a30+c93, a30+c94, a30+c95, a30+c96, a30+c97, a30+c98, a30+c99, a30+c100, a30+c101, a30+c102, a30+c103, a30+c104, a30+c105, a30+c106, a30+c107, a30+c108, a30+c109, a30+c110, a30+c111, a30+c112, a30+c113, a30+c114, a30+c115, a30+c116, a30+c117, a30+c118, a30+c119, a30+c120, a30+c121, a30+c122, a30+c123, a30+c124, a30+c125, a30+c126, a30+c127, a30+c128, a30+c129, a30+c130, a30+c131, a30+c132, a30+c133, a30+c134, a30+c135, a30+c136, a30+c137, a30+c138, a30+c139, a30+c140, a30+c141, a30+c142, a30+c143, a30+c144, a30+c145, a30+c146, a30+c147, a30+c148, a30+c149, a30+c150, a30+c151, a30+c152, a30+c153, a30+c154, a30+c155, a30+c156, a30+c157, a30+c158, a30+c159, a30+c160, a30+c161, a30+c162, a30+c163, a30+c164, a30+c165, a30+c166, a30+c167, a30+c168, a30+c169, a30+c170, a30+c171, a30+c172, a30+c173, a30+c174, a30+c175, a30+c176, a30+c177, a30+c178, a30+c179, a30+c180, a30+c181, a30+c182, a30+c183, a30+c184, a30+c185, a30+c186, a30+c187, a30+c188, a30+c189, a30+c190, a30+c191, a30+c192, a30+c193, a30+c194, a30+c195, a30+c196, a30+c197, a30+c198, a30+c199, a30+c200, a30+c201, a30+c202, a30+c203, a30+c204, a30+c205, a30+c206, a30+c207, a30+c208, a30+c209, a30+c210, a30+c211, a31+c1, a31+c2, a31+c3, a31+c4, a31+c5, a31+c6, a31+c7, a31+c8, a31+c9, a31+c10, a31+c11, a31+c12, a31+c13, a31+c14, a31+c15, a31+c16, a31+c17, a31+c18, a31+c19, a31+c20, a31+c21, a31+c22, a31+c23, a31+c24, a31+c25, a31+c26, a31+c27, a31+c28, a31+c29, a31+c30, a31+c31, a31+c32, a31+c33, a31+c34, a31+c35, a31+c36, a31+c37, a31+c38, a31+c39, a31+c40, a31+c41, a31+c42, a31+c43, a31+c44, a31+c45, a31+c46, a31+c47, a31+c48, a31+c49, a31+c50, a31+c51, a31+c52, a31+c53, a31+c54, a31+c55, a31+c56, a31+c57, a31+c58, a31+c59, a31+c60, a31+c61, a31+c62, a31+c63, a31+c64, a31+c65, a31+c66, a31+c67, a31+c68, a31+c69, a31+c70, a31+c71, a31+c72, a31+c73, a31+c74, a31+c75, a31+c76, a31+c77, a31+c78, a31+c79, a31+c80, a31+c81, a31+c82, a31+c83, a31+c84, a31+c85, a31+c86, a31+c87, a31+c88, a31+c89, a31+c90, a31+c91, a31+c92, a31+c93, a31+c94, a31+c95, a31+c96, a31+c97, a31+c98, a31+c99, a31+c100, a31+c101, a31+c102, a31+c103, a31+c104, a31+c105, a31+c106, a31+c107, a31+c108, a31+c109, a31+c110, a31+c111, a31+c112, a31+c113, a31+c114, a31+c115, a31+c116, a31+c117, a31+c118, a31+c119, a31+c120, a31+c121, a31+c122, a31+c123, a31+c124, a31+c125, a31+c126, a31+c127, a31+c128, a31+c129, a31+c130, a31+c131, a31+c132, a31+c133, a31+c134, a31+c135, a31+c136, a31+c137, a31+c138, a31+c139, a31+c140, a31+c141, a31+c142, a31+c143, a31+c144, a31+c145, a31+c146, a31+c147, a31+c148, a31+c149, a31+c150, a31+c151, a31+c152, a31+c153, a31+c154, a31+c155, a31+c156, a31+c157, a31+c158, a31+c159, a31+c160, a31+c161, a31+c162, a31+c163, a31+c164, a31+c165, a31+c166, a31+c167, a31+c168, a31+c169, a31+c170, a31+c171, a31+c172, a31+c173, a31+c174, a31+c175, a31+c176, a31+c177, a31+c178, a31+c179, a31+c180, a31+c181, a31+c182, a31+c183, a31+c184, a31+c185, a31+c186, a31+c187, a31+c188, a31+c189, a31+c190, a31+c191, a31+c192, a31+c193, a31+c194, a31+c195, a31+c196, a31+c197, a31+c198, a31+c199, a31+c200, a31+c201, a31+c202, a31+c203, a31+c204, a31+c205, a31+c206, a31+c207, a31+c208, a31+c209, a31+c210, a31+c211, a32+c1, a32+c2, a32+c3, a32+c4, a32+c5, a32+c6, a32+c7, a32+c8, a32+c9, a32+c10, a32+c11, a32+c12, a32+c13, a32+c14, a32+c15, a32+c16, a32+c17, a32+c18, a32+c19, a32+c20, a32+c21, a32+c22, a32+c23, a32+c24, a32+c25, a32+c26, a32+c27, a32+c28, a32+c29, a32+c30, a32+c31, a32+c32, a32+c33, a32+c34, a32+c35, a32+c36, a32+c37, a32+c38, a32+c39, a32+c40, a32+c41, a32+c42, a32+c43, a32+c44, a32+c45, a32+c46, a32+c47, a32+c48, a32+c49, a32+c50, a32+c51, a32+c52, a32+c53, a32+c54, a32+c55, a32+c56, a32+c57, a32+c58, a32+c59, a32+c60, a32+c61, a32+c62, a32+c63, a32+c64, a32+c65, a32+c66, a32+c67, a32+c68, a32+c69, a32+c70, a32+c71, a32+c72, a32+c73, a32+c74, a32+c75, a32+c76, a32+c77, a32+c78, a32+c79, a32+c80, a32+c81, a32+c82, a32+c83, a32+c84, a32+c85, a32+c86, a32+c87, a32+c88, a32+c89, a32+c90, a32+c91, a32+c92, a32+c93, a32+c94, a32+c95, a32+c96, a32+c97, a32+c98, a32+c99, a32+c100, a32+c101, a32+c102, a32+c103, a32+c104, a32+c105, a32+c106, a32+c107, a32+c108, a32+c109, a32+c110, a32+c111, a32+c112, a32+c113, a32+c114, a32+c115, a32+c116, a32+c117, a32+c118, a32+c119, a32+c120, a32+c121, a32+c122, a32+c123, a32+c124, a32+c125, a32+c126, a32+c127, a32+c128, a32+c129, a32+c130, a32+c131, a32+c132, a32+c133, a32+c134, a32+c135, a32+c136, a32+c137, a32+c138, a32+c139, a32+c140, a32+c141, a32+c142, a32+c143, a32+c144, a32+c145, a32+c146, a32+c147, a32+c148, a32+c149, a32+c150, a32+c151, a32+c152, a32+c153, a32+c154, a32+c155, a32+c156, a32+c157, a32+c158, a32+c159, a32+c160, a32+c161, a32+c162, a32+c163, a32+c164, a32+c165, a32+c166, a32+c167, a32+c168, a32+c169, a32+c170, a32+c171, a32+c172, a32+c173, a32+c174, a32+c175, a32+c176, a32+c177, a32+c178, a32+c179, a32+c180, a32+c181, a32+c182, a32+c183, a32+c184, a32+c185, a32+c186, a32+c187, a32+c188, a32+c189, a32+c190, a32+c191, a32+c192, a32+c193, a32+c194, a32+c195, a32+c196, a32+c197, a32+c198, a32+c199, a32+c200, a32+c201, a32+c202, a32+c203, a32+c204, a32+c205, a32+c206, a32+c207, a32+c208, a32+c209, a32+c210, a32+c211, a33+c1, a33+c2, a33+c3, a33+c4, a33+c5, a33+c6, a33+c7, a33+c8, a33+c9, a33+c10, a33+c11, a33+c12, a33+c13, a33+c14, a33+c15, a33+c16, a33+c17, a33+c18, a33+c19, a33+c20, a33+c21, a33+c22, a33+c23, a33+c24, a33+c25, a33+c26, a33+c27, a33+c28, a33+c29, a33+c30, a33+c31, a33+c32, a33+c33, a33+c34, a33+c35, a33+c36, a33+c37, a33+c38, a33+c39, a33+c40, a33+c41, a33+c42, a33+c43, a33+c44, a33+c45, a33+c46, a33+c47, a33+c48, a33+c49, a33+c50, a33+c51, a33+c52, a33+c53, a33+c54, a33+c55, a33+c56, a33+c57, a33+c58, a33+c59, a33+c60, a33+c61, a33+c62, a33+c63, a33+c64, a33+c65, a33+c66, a33+c67, a33+c68, a33+c69, a33+c70, a33+c71, a33+c72, a33+c73, a33+c74, a33+c75, a33+c76, a33+c77, a33+c78, a33+c79, a33+c80, a33+c81, a33+c82, a33+c83, a33+c84, a33+c85, a33+c86, a33+c87, a33+c88, a33+c89, a33+c90, a33+c91, a33+c92, a33+c93, a33+c94, a33+c95, a33+c96, a33+c97, a33+c98, a33+c99, a33+c100, a33+c101, a33+c102, a33+c103, a33+c104, a33+c105, a33+c106, a33+c107, a33+c108, a33+c109, a33+c110, a33+c111, a33+c112, a33+c113, a33+c114, a33+c115, a33+c116, a33+c117, a33+c118, a33+c119, a33+c120, a33+c121, a33+c122, a33+c123, a33+c124, a33+c125, a33+c126, a33+c127, a33+c128, a33+c129, a33+c130, a33+c131, a33+c132, a33+c133, a33+c134, a33+c135, a33+c136, a33+c137, a33+c138, a33+c139, a33+c140, a33+c141, a33+c142, a33+c143, a33+c144, a33+c145, a33+c146, a33+c147, a33+c148, a33+c149, a33+c150, a33+c151, a33+c152, a33+c153, a33+c154, a33+c155, a33+c156, a33+c157, a33+c158, a33+c159, a33+c160, a33+c161, a33+c162, a33+c163, a33+c164, a33+c165, a33+c166, a33+c167, a33+c168, a33+c169, a33+c170, a33+c171, a33+c172, a33+c173, a33+c174, a33+c175, a33+c176, a33+c177, a33+c178, a33+c179, a33+c180, a33+c181, a33+c182, a33+c183, a33+c184, a33+c185, a33+c186, a33+c187, a33+c188, a33+c189, a33+c190, a33+c191, a33+c192, a33+c193, a33+c194, a33+c195, a33+c196, a33+c197, a33+c198, a33+c199, a33+c200, a33+c201, a33+c202, a33+c203, a33+c204, a33+c205, a33+c206, a33+c207, a33+c208, a33+c209, a33+c210, a33+c211, a34+c1, a34+c2, a34+c3, a34+c4, a34+c5, a34+c6, a34+c7, a34+c8, a34+c9, a34+c10, a34+c11, a34+c12, a34+c13, a34+c14, a34+c15, a34+c16, a34+c17, a34+c18, a34+c19, a34+c20, a34+c21, a34+c22, a34+c23, a34+c24, a34+c25, a34+c26, a34+c27, a34+c28, a34+c29, a34+c30, a34+c31, a34+c32, a34+c33, a34+c34, a34+c35, a34+c36, a34+c37, a34+c38, a34+c39, a34+c40, a34+c41, a34+c42, a34+c43, a34+c44, a34+c45, a34+c46, a34+c47, a34+c48, a34+c49, a34+c50, a34+c51, a34+c52, a34+c53, a34+c54, a34+c55, a34+c56, a34+c57, a34+c58, a34+c59, a34+c60, a34+c61, a34+c62, a34+c63, a34+c64, a34+c65, a34+c66, a34+c67, a34+c68, a34+c69, a34+c70, a34+c71, a34+c72, a34+c73, a34+c74, a34+c75, a34+c76, a34+c77, a34+c78, a34+c79, a34+c80, a34+c81, a34+c82, a34+c83, a34+c84, a34+c85, a34+c86, a34+c87, a34+c88, a34+c89, a34+c90, a34+c91, a34+c92, a34+c93, a34+c94, a34+c95, a34+c96, a34+c97, a34+c98, a34+c99, a34+c100, a34+c101, a34+c102, a34+c103, a34+c104, a34+c105, a34+c106, a34+c107, a34+c108, a34+c109, a34+c110, a34+c111, a34+c112, a34+c113, a34+c114, a34+c115, a34+c116, a34+c117, a34+c118, a34+c119, a34+c120, a34+c121, a34+c122, a34+c123, a34+c124, a34+c125, a34+c126, a34+c127, a34+c128, a34+c129, a34+c130, a34+c131, a34+c132, a34+c133, a34+c134, a34+c135, a34+c136, a34+c137, a34+c138, a34+c139, a34+c140, a34+c141, a34+c142, a34+c143, a34+c144, a34+c145, a34+c146, a34+c147, a34+c148, a34+c149, a34+c150, a34+c151, a34+c152, a34+c153, a34+c154, a34+c155, a34+c156, a34+c157, a34+c158, a34+c159, a34+c160, a34+c161, a34+c162, a34+c163, a34+c164, a34+c165, a34+c166, a34+c167, a34+c168, a34+c169, a34+c170, a34+c171, a34+c172, a34+c173, a34+c174, a34+c175, a34+c176, a34+c177, a34+c178, a34+c179, a34+c180, a34+c181, a34+c182, a34+c183, a34+c184, a34+c185, a34+c186, a34+c187, a34+c188, a34+c189, a34+c190, a34+c191, a34+c192, a34+c193, a34+c194, a34+c195, a34+c196, a34+c197, a34+c198, a34+c199, a34+c200, a34+c201, a34+c202, a34+c203, a34+c204, a34+c205, a34+c206, a34+c207, a34+c208, a34+c209, a34+c210, a34+c211, a35+c1, a35+c2, a35+c3, a35+c4, a35+c5, a35+c6, a35+c7, a35+c8, a35+c9, a35+c10, a35+c11, a35+c12, a35+c13, a35+c14, a35+c15, a35+c16, a35+c17, a35+c18, a35+c19, a35+c20, a35+c21, a35+c22, a35+c23, a35+c24, a35+c25, a35+c26, a35+c27, a35+c28, a35+c29, a35+c30, a35+c31, a35+c32, a35+c33, a35+c34, a35+c35, a35+c36, a35+c37, a35+c38, a35+c39, a35+c40, a35+c41, a35+c42, a35+c43, a35+c44, a35+c45, a35+c46, a35+c47, a35+c48, a35+c49, a35+c50, a35+c51, a35+c52, a35+c53, a35+c54, a35+c55, a35+c56, a35+c57, a35+c58, a35+c59, a35+c60, a35+c61, a35+c62, a35+c63, a35+c64, a35+c65, a35+c66, a35+c67, a35+c68, a35+c69, a35+c70, a35+c71, a35+c72, a35+c73, a35+c74, a35+c75, a35+c76, a35+c77, a35+c78, a35+c79, a35+c80, a35+c81, a35+c82, a35+c83, a35+c84, a35+c85, a35+c86, a35+c87, a35+c88, a35+c89, a35+c90, a35+c91, a35+c92, a35+c93, a35+c94, a35+c95, a35+c96, a35+c97, a35+c98, a35+c99, a35+c100, a35+c101, a35+c102, a35+c103, a35+c104, a35+c105, a35+c106, a35+c107, a35+c108, a35+c109, a35+c110, a35+c111, a35+c112, a35+c113, a35+c114, a35+c115, a35+c116, a35+c117, a35+c118, a35+c119, a35+c120, a35+c121, a35+c122, a35+c123, a35+c124, a35+c125, a35+c126, a35+c127, a35+c128, a35+c129, a35+c130, a35+c131, a35+c132, a35+c133, a35+c134, a35+c135, a35+c136, a35+c137, a35+c138, a35+c139, a35+c140, a35+c141, a35+c142, a35+c143, a35+c144, a35+c145, a35+c146, a35+c147, a35+c148, a35+c149, a35+c150, a35+c151, a35+c152, a35+c153, a35+c154, a35+c155, a35+c156, a35+c157, a35+c158, a35+c159, a35+c160, a35+c161, a35+c162, a35+c163, a35+c164, a35+c165, a35+c166, a35+c167, a35+c168, a35+c169, a35+c170, a35+c171, a35+c172, a35+c173, a35+c174, a35+c175, a35+c176, a35+c177, a35+c178, a35+c179, a35+c180, a35+c181, a35+c182, a35+c183, a35+c184, a35+c185, a35+c186, a35+c187, a35+c188, a35+c189, a35+c190, a35+c191, a35+c192, a35+c193, a35+c194, a35+c195, a35+c196, a35+c197, a35+c198, a35+c199, a35+c200, a35+c201, a35+c202, a35+c203, a35+c204, a35+c205, a35+c206, a35+c207, a35+c208, a35+c209, a35+c210, a35+c211, a36+c1, a36+c2, a36+c3, a36+c4, a36+c5, a36+c6, a36+c7, a36+c8, a36+c9, a36+c10, a36+c11, a36+c12, a36+c13, a36+c14, a36+c15, a36+c16, a36+c17, a36+c18, a36+c19, a36+c20, a36+c21, a36+c22, a36+c23, a36+c24, a36+c25, a36+c26, a36+c27, a36+c28, a36+c29, a36+c30, a36+c31, a36+c32, a36+c33, a36+c34, a36+c35, a36+c36, a36+c37, a36+c38, a36+c39, a36+c40, a36+c41, a36+c42, a36+c43, a36+c44, a36+c45, a36+c46, a36+c47, a36+c48, a36+c49, a36+c50, a36+c51, a36+c52, a36+c53, a36+c54, a36+c55, a36+c56, a36+c57, a36+c58, a36+c59, a36+c60, a36+c61, a36+c62, a36+c63, a36+c64, a36+c65, a36+c66, a36+c67, a36+c68, a36+c69, a36+c70, a36+c71, a36+c72, a36+c73, a36+c74, a36+c75, a36+c76, a36+c77, a36+c78, a36+c79, a36+c80, a36+c81, a36+c82, a36+c83, a36+c84, a36+c85, a36+c86, a36+c87, a36+c88, a36+c89, a36+c90, a36+c91, a36+c92, a36+c93, a36+c94, a36+c95, a36+c96, a36+c97, a36+c98, a36+c99, a36+c100, a36+c101, a36+c102, a36+c103, a36+c104, a36+c105, a36+c106, a36+c107, a36+c108, a36+c109, a36+c110, a36+c111, a36+c112, a36+c113, a36+c114, a36+c115, a36+c116, a36+c117, a36+c118, a36+c119, a36+c120, a36+c121, a36+c122, a36+c123, a36+c124, a36+c125, a36+c126, a36+c127, a36+c128, a36+c129, a36+c130, a36+c131, a36+c132, a36+c133, a36+c134, a36+c135, a36+c136, a36+c137, a36+c138, a36+c139, a36+c140, a36+c141, a36+c142, a36+c143, a36+c144, a36+c145, a36+c146, a36+c147, a36+c148, a36+c149, a36+c150, a36+c151, a36+c152, a36+c153, a36+c154, a36+c155, a36+c156, a36+c157, a36+c158, a36+c159, a36+c160, a36+c161, a36+c162, a36+c163, a36+c164, a36+c165, a36+c166, a36+c167, a36+c168, a36+c169, a36+c170, a36+c171, a36+c172, a36+c173, a36+c174, a36+c175, a36+c176, a36+c177, a36+c178, a36+c179, a36+c180, a36+c181, a36+c182, a36+c183, a36+c184, a36+c185, a36+c186, a36+c187, a36+c188, a36+c189, a36+c190, a36+c191, a36+c192, a36+c193, a36+c194, a36+c195, a36+c196, a36+c197, a36+c198, a36+c199, a36+c200, a36+c201, a36+c202, a36+c203, a36+c204, a36+c205, a36+c206, a36+c207, a36+c208, a36+c209, a36+c210, a36+c211, a37+c1, a37+c2, a37+c3, a37+c4, a37+c5, a37+c6, a37+c7, a37+c8, a37+c9, a37+c10, a37+c11, a37+c12, a37+c13, a37+c14, a37+c15, a37+c16, a37+c17, a37+c18, a37+c19, a37+c20, a37+c21, a37+c22, a37+c23, a37+c24, a37+c25, a37+c26, a37+c27, a37+c28, a37+c29, a37+c30, a37+c31, a37+c32, a37+c33, a37+c34, a37+c35, a37+c36, a37+c37, a37+c38, a37+c39, a37+c40, a37+c41, a37+c42, a37+c43, a37+c44, a37+c45, a37+c46, a37+c47, a37+c48, a37+c49, a37+c50, a37+c51, a37+c52, a37+c53, a37+c54, a37+c55, a37+c56, a37+c57, a37+c58, a37+c59, a37+c60, a37+c61, a37+c62, a37+c63, a37+c64, a37+c65, a37+c66, a37+c67, a37+c68, a37+c69, a37+c70, a37+c71, a37+c72, a37+c73, a37+c74, a37+c75, a37+c76, a37+c77, a37+c78, a37+c79, a37+c80, a37+c81, a37+c82, a37+c83, a37+c84, a37+c85, a37+c86, a37+c87, a37+c88, a37+c89, a37+c90, a37+c91, a37+c92, a37+c93, a37+c94, a37+c95, a37+c96, a37+c97, a37+c98, a37+c99, a37+c100, a37+c101, a37+c102, a37+c103, a37+c104, a37+c105, a37+c106, a37+c107, a37+c108, a37+c109, a37+c110, a37+c111, a37+c112, a37+c113, a37+c114, a37+c115, a37+c116, a37+c117, a37+c118, a37+c119, a37+c120, a37+c121, a37+c122, a37+c123, a37+c124, a37+c125, a37+c126, a37+c127, a37+c128, a37+c129, a37+c130, a37+c131, a37+c132, a37+c133, a37+c134, a37+c135, a37+c136, a37+c137, a37+c138, a37+c139, a37+c140, a37+c141, a37+c142, a37+c143, a37+c144, a37+c145, a37+c146, a37+c147, a37+c148, a37+c149, a37+c150, a37+c151, a37+c152, a37+c153, a37+c154, a37+c155, a37+c156, a37+c157, a37+c158, a37+c159, a37+c160, a37+c161, a37+c162, a37+c163, a37+c164, a37+c165, a37+c166, a37+c167, a37+c168, a37+c169, a37+c170, a37+c171, a37+c172, a37+c173, a37+c174, a37+c175, a37+c176, a37+c177, a37+c178, a37+c179, a37+c180, a37+c181, a37+c182, a37+c183, a37+c184, a37+c185, a37+c186, a37+c187, a37+c188, a37+c189, a37+c190, a37+c191, a37+c192, a37+c193, a37+c194, a37+c195, a37+c196, a37+c197, a37+c198, a37+c199, a37+c200, a37+c201, a37+c202, a37+c203, a37+c204, a37+c205, a37+c206, a37+c207, a37+c208, a37+c209, a37+c210, a37+c211, a38+c1, a38+c2, a38+c3, a38+c4, a38+c5, a38+c6, a38+c7, a38+c8, a38+c9, a38+c10, a38+c11, a38+c12, a38+c13, a38+c14, a38+c15, a38+c16, a38+c17, a38+c18, a38+c19, a38+c20, a38+c21, a38+c22, a38+c23, a38+c24, a38+c25, a38+c26, a38+c27, a38+c28, a38+c29, a38+c30, a38+c31, a38+c32, a38+c33, a38+c34, a38+c35, a38+c36, a38+c37, a38+c38, a38+c39, a38+c40, a38+c41, a38+c42, a38+c43, a38+c44, a38+c45, a38+c46, a38+c47, a38+c48, a38+c49, a38+c50, a38+c51, a38+c52, a38+c53, a38+c54, a38+c55, a38+c56, a38+c57, a38+c58, a38+c59, a38+c60, a38+c61, a38+c62, a38+c63, a38+c64, a38+c65, a38+c66, a38+c67, a38+c68, a38+c69, a38+c70, a38+c71, a38+c72, a38+c73, a38+c74, a38+c75, a38+c76, a38+c77, a38+c78, a38+c79, a38+c80, a38+c81, a38+c82, a38+c83, a38+c84, a38+c85, a38+c86, a38+c87, a38+c88, a38+c89, a38+c90, a38+c91, a38+c92, a38+c93, a38+c94, a38+c95, a38+c96, a38+c97, a38+c98, a38+c99, a38+c100, a38+c101, a38+c102, a38+c103, a38+c104, a38+c105, a38+c106, a38+c107, a38+c108, a38+c109, a38+c110, a38+c111, a38+c112, a38+c113, a38+c114, a38+c115, a38+c116, a38+c117, a38+c118, a38+c119, a38+c120, a38+c121, a38+c122, a38+c123, a38+c124, a38+c125, a38+c126, a38+c127, a38+c128, a38+c129, a38+c130, a38+c131, a38+c132, a38+c133, a38+c134, a38+c135, a38+c136, a38+c137, a38+c138, a38+c139, a38+c140, a38+c141, a38+c142, a38+c143, a38+c144, a38+c145, a38+c146, a38+c147, a38+c148, a38+c149, a38+c150, a38+c151, a38+c152, a38+c153, a38+c154, a38+c155, a38+c156, a38+c157, a38+c158, a38+c159, a38+c160, a38+c161, a38+c162, a38+c163, a38+c164, a38+c165, a38+c166, a38+c167, a38+c168, a38+c169, a38+c170, a38+c171, a38+c172, a38+c173, a38+c174, a38+c175, a38+c176, a38+c177, a38+c178, a38+c179, a38+c180, a38+c181, a38+c182, a38+c183, a38+c184, a38+c185, a38+c186, a38+c187, a38+c188, a38+c189, a38+c190, a38+c191, a38+c192, a38+c193, a38+c194, a38+c195, a38+c196, a38+c197, a38+c198, a38+c199, a38+c200, a38+c201, a38+c202, a38+c203, a38+c204, a38+c205, a38+c206, a38+c207, a38+c208, a38+c209, a38+c210, a38+c211, a39+c1, a39+c2, a39+c3, a39+c4, a39+c5, a39+c6, a39+c7, a39+c8, a39+c9, a39+c10, a39+c11, a39+c12, a39+c13, a39+c14, a39+c15, a39+c16, a39+c17, a39+c18, a39+c19, a39+c20, a39+c21, a39+c22, a39+c23, a39+c24, a39+c25, a39+c26, a39+c27, a39+c28, a39+c29, a39+c30, a39+c31, a39+c32, a39+c33, a39+c34, a39+c35, a39+c36, a39+c37, a39+c38, a39+c39, a39+c40, a39+c41, a39+c42, a39+c43, a39+c44, a39+c45, a39+c46, a39+c47, a39+c48, a39+c49, a39+c50, a39+c51, a39+c52, a39+c53, a39+c54, a39+c55, a39+c56, a39+c57, a39+c58, a39+c59, a39+c60, a39+c61, a39+c62, a39+c63, a39+c64, a39+c65, a39+c66, a39+c67, a39+c68, a39+c69, a39+c70, a39+c71, a39+c72, a39+c73, a39+c74, a39+c75, a39+c76, a39+c77, a39+c78, a39+c79, a39+c80, a39+c81, a39+c82, a39+c83, a39+c84, a39+c85, a39+c86, a39+c87, a39+c88, a39+c89, a39+c90, a39+c91, a39+c92, a39+c93, a39+c94, a39+c95, a39+c96, a39+c97, a39+c98, a39+c99, a39+c100, a39+c101, a39+c102, a39+c103, a39+c104, a39+c105, a39+c106, a39+c107, a39+c108, a39+c109, a39+c110, a39+c111, a39+c112, a39+c113, a39+c114, a39+c115, a39+c116, a39+c117, a39+c118, a39+c119, a39+c120, a39+c121, a39+c122, a39+c123, a39+c124, a39+c125, a39+c126, a39+c127, a39+c128, a39+c129, a39+c130, a39+c131, a39+c132, a39+c133, a39+c134, a39+c135, a39+c136, a39+c137, a39+c138, a39+c139, a39+c140, a39+c141, a39+c142, a39+c143, a39+c144, a39+c145, a39+c146, a39+c147, a39+c148, a39+c149, a39+c150, a39+c151, a39+c152, a39+c153, a39+c154, a39+c155, a39+c156, a39+c157, a39+c158, a39+c159, a39+c160, a39+c161, a39+c162, a39+c163, a39+c164, a39+c165, a39+c166, a39+c167, a39+c168, a39+c169, a39+c170, a39+c171, a39+c172, a39+c173, a39+c174, a39+c175, a39+c176, a39+c177, a39+c178, a39+c179, a39+c180, a39+c181, a39+c182, a39+c183, a39+c184, a39+c185, a39+c186, a39+c187, a39+c188, a39+c189, a39+c190, a39+c191, a39+c192, a39+c193, a39+c194, a39+c195, a39+c196, a39+c197, a39+c198, a39+c199, a39+c200, a39+c201, a39+c202, a39+c203, a39+c204, a39+c205, a39+c206, a39+c207, a39+c208, a39+c209, a39+c210, a39+c211, a40+c1, a40+c2, a40+c3, a40+c4, a40+c5, a40+c6, a40+c7, a40+c8, a40+c9, a40+c10, a40+c11, a40+c12, a40+c13, a40+c14, a40+c15, a40+c16, a40+c17, a40+c18, a40+c19, a40+c20, a40+c21, a40+c22, a40+c23, a40+c24, a40+c25, a40+c26, a40+c27, a40+c28, a40+c29, a40+c30, a40+c31, a40+c32, a40+c33, a40+c34, a40+c35, a40+c36, a40+c37, a40+c38, a40+c39, a40+c40, a40+c41, a40+c42, a40+c43, a40+c44, a40+c45, a40+c46, a40+c47, a40+c48, a40+c49, a40+c50, a40+c51, a40+c52, a40+c53, a40+c54, a40+c55, a40+c56, a40+c57, a40+c58, a40+c59, a40+c60, a40+c61, a40+c62, a40+c63, a40+c64, a40+c65, a40+c66, a40+c67, a40+c68, a40+c69, a40+c70, a40+c71, a40+c72, a40+c73, a40+c74, a40+c75, a40+c76, a40+c77, a40+c78, a40+c79, a40+c80, a40+c81, a40+c82, a40+c83, a40+c84, a40+c85, a40+c86, a40+c87, a40+c88, a40+c89, a40+c90, a40+c91, a40+c92, a40+c93, a40+c94, a40+c95, a40+c96, a40+c97, a40+c98, a40+c99, a40+c100, a40+c101, a40+c102, a40+c103, a40+c104, a40+c105, a40+c106, a40+c107, a40+c108, a40+c109, a40+c110, a40+c111, a40+c112, a40+c113, a40+c114, a40+c115, a40+c116, a40+c117, a40+c118, a40+c119, a40+c120, a40+c121, a40+c122, a40+c123, a40+c124, a40+c125, a40+c126, a40+c127, a40+c128, a40+c129, a40+c130, a40+c131, a40+c132, a40+c133, a40+c134, a40+c135, a40+c136, a40+c137, a40+c138, a40+c139, a40+c140, a40+c141, a40+c142, a40+c143, a40+c144, a40+c145, a40+c146, a40+c147, a40+c148, a40+c149, a40+c150, a40+c151, a40+c152, a40+c153, a40+c154, a40+c155, a40+c156, a40+c157, a40+c158, a40+c159, a40+c160, a40+c161, a40+c162, a40+c163, a40+c164, a40+c165, a40+c166, a40+c167, a40+c168, a40+c169, a40+c170, a40+c171, a40+c172, a40+c173, a40+c174, a40+c175, a40+c176, a40+c177, a40+c178, a40+c179, a40+c180, a40+c181, a40+c182, a40+c183, a40+c184, a40+c185, a40+c186, a40+c187, a40+c188, a40+c189, a40+c190, a40+c191, a40+c192, a40+c193, a40+c194, a40+c195, a40+c196, a40+c197, a40+c198, a40+c199, a40+c200, a40+c201, a40+c202, a40+c203, a40+c204, a40+c205, a40+c206, a40+c207, a40+c208, a40+c209, a40+c210, a40+c211, a41+c1, a41+c2, a41+c3, a41+c4, a41+c5, a41+c6, a41+c7, a41+c8, a41+c9, a41+c10, a41+c11, a41+c12, a41+c13, a41+c14, a41+c15, a41+c16, a41+c17, a41+c18, a41+c19, a41+c20, a41+c21, a41+c22, a41+c23, a41+c24, a41+c25, a41+c26, a41+c27, a41+c28, a41+c29, a41+c30, a41+c31, a41+c32, a41+c33, a41+c34, a41+c35, a41+c36, a41+c37, a41+c38, a41+c39, a41+c40, a41+c41, a41+c42, a41+c43, a41+c44, a41+c45, a41+c46, a41+c47, a41+c48, a41+c49, a41+c50, a41+c51, a41+c52, a41+c53, a41+c54, a41+c55, a41+c56, a41+c57, a41+c58, a41+c59, a41+c60, a41+c61, a41+c62, a41+c63, a41+c64, a41+c65, a41+c66, a41+c67, a41+c68, a41+c69, a41+c70, a41+c71, a41+c72, a41+c73, a41+c74, a41+c75, a41+c76, a41+c77, a41+c78, a41+c79, a41+c80, a41+c81, a41+c82, a41+c83, a41+c84, a41+c85, a41+c86, a41+c87, a41+c88, a41+c89, a41+c90, a41+c91, a41+c92, a41+c93, a41+c94, a41+c95, a41+c96, a41+c97, a41+c98, a41+c99, a41+c100, a41+c101, a41+c102, a41+c103, a41+c104, a41+c105, a41+c106, a41+c107, a41+c108, a41+c109, a41+c110, a41+c111, a41+c112, a41+c113, a41+c114, a41+c115, a41+c116, a41+c117, a41+c118, a41+c119, a41+c120, a41+c121, a41+c122, a41+c123, a41+c124, a41+c125, a41+c126, a41+c127, a41+c128, a41+c129, a41+c130, a41+c131, a41+c132, a41+c133, a41+c134, a41+c135, a41+c136, a41+c137, a41+c138, a41+c139, a41+c140, a41+c141, a41+c142, a41+c143, a41+c144, a41+c145, a41+c146, a41+c147, a41+c148, a41+c149, a41+c150, a41+c151, a41+c152, a41+c153, a41+c154, a41+c155, a41+c156, a41+c157, a41+c158, a41+c159, a41+c160, a41+c161, a41+c162, a41+c163, a41+c164, a41+c165, a41+c166, a41+c167, a41+c168, a41+c169, a41+c170, a41+c171, a41+c172, a41+c173, a41+c174, a41+c175, a41+c176, a41+c177, a41+c178, a41+c179, a41+c180, a41+c181, a41+c182, a41+c183, a41+c184, a41+c185, a41+c186, a41+c187, a41+c188, a41+c189, a41+c190, a41+c191, a41+c192, a41+c193, a41+c194, a41+c195, a41+c196, a41+c197, a41+c198, a41+c199, a41+c200, a41+c201, a41+c202, a41+c203, a41+c204, a41+c205, a41+c206, a41+c207, a41+c208, a41+c209, a41+c210, a41+c211, a42+c1, a42+c2, a42+c3, a42+c4, a42+c5, a42+c6, a42+c7, a42+c8, a42+c9, a42+c10, a42+c11, a42+c12, a42+c13, a42+c14, a42+c15, a42+c16, a42+c17, a42+c18, a42+c19, a42+c20, a42+c21, a42+c22, a42+c23, a42+c24, a42+c25, a42+c26, a42+c27, a42+c28, a42+c29, a42+c30, a42+c31, a42+c32, a42+c33, a42+c34, a42+c35, a42+c36, a42+c37, a42+c38, a42+c39, a42+c40, a42+c41, a42+c42, a42+c43, a42+c44, a42+c45, a42+c46, a42+c47, a42+c48, a42+c49, a42+c50, a42+c51, a42+c52, a42+c53, a42+c54, a42+c55, a42+c56, a42+c57, a42+c58, a42+c59, a42+c60, a42+c61, a42+c62, a42+c63, a42+c64, a42+c65, a42+c66, a42+c67, a42+c68, a42+c69, a42+c70, a42+c71, a42+c72, a42+c73, a42+c74, a42+c75, a42+c76, a42+c77, a42+c78, a42+c79, a42+c80, a42+c81, a42+c82, a42+c83, a42+c84, a42+c85, a42+c86, a42+c87, a42+c88, a42+c89, a42+c90, a42+c91, a42+c92, a42+c93, a42+c94, a42+c95, a42+c96, a42+c97, a42+c98, a42+c99, a42+c100, a42+c101, a42+c102, a42+c103, a42+c104, a42+c105, a42+c106, a42+c107, a42+c108, a42+c109, a42+c110, a42+c111, a42+c112, a42+c113,a42+c114, a42+c115,a42+c116, a42+c117, a42+c118, a42+c119, a42+c120, a42+c121, a42+c122, a42+c123, a42+c124, a42+c125, a42+c126, a42+c127, a42+c128, a42+c129, a42+c130, a42+c131, a42+c132, a42+c133, a42+c134, a42+c135, a42+c136, a42+c137, a42+c138, a42+c139, a42+c140, a42+c141, a42+c142, a42+c143, a42+c144, a42+c145, a42+c146, a42+c147, a42+c148, a42+c149, a42+c150, a42+c151, a42+c152, a42+c153, a42+c154, a42+c155, a42+c156, a42+c157, a42+c158, a42+c159, a42+c160, a42+c161, a42+c162, a42+c163, a42+c164, a42+c165, a42+c166, a42+c167, a42+c168, a42+c169, a42+c170, a42+c171, a42+c172, a42+c173, a42+c174, a42+c175, a42+c176, a42+c177, a42+c178, a42+c179, a42+c180, a42+c181, a42+c182, a42+c183, a42+c184, a42+c185, a42+c186, a42+c187, a42+c188, a42+c189, a42+c190, a42+c191, a42+c192, a42+c193, a42+c194, a42+c195, a42+c196, a42+c197, a42+c198, a42+c199, a42+c200, a42+c201, a42+c202, a42+c203, a42+c204, a42+c205, a42+c206, a42+c207, a42+c208, a42+c209, a42+c210, a42+c211, a43+c1, a43+c2, a43+c3, a43+c4, a43+c5, a43+c6, a43+c7, a43+c8, a43+c9, a43+c10, a43+c11, a43+c12, a43+c13, a43+c14, a43+c15, a43+c16, a43+c17, a43+c18, a43+c19, a43+c20, a43+c21, a43+c22, a43+c23, a43+c24, a43+c25, a43+c26, a43+c27, a43+c28, a43+c29, a43+c30, a43+c31, a43+c32, a43+c33, a43+c34, a43+c35, a43+c36, a43+c37, a43+c38, a43+c39, a43+c40, a43+c41, a43+c42, a43+c43, a43+c44, a43+c45, a43+c46, a43+c47, a43+c48, a43+c49, a43+c50, a43+c51, a43+c52, a43+c53, a43+c54, a43+c55, a43+c56, a43+c57, a43+c58, a43+c59, a43+c60, a43+c61, a43+c62, a43+c63, a43+c64, a43+c65, a43+c66, a43+c67, a43+c68, a43+c69, a43+c70, a43+c71, a43+c72, a43+c73, a43+c74, a43+c75, a43+c76, a43+c77, a43+c78, a43+c79, a43+c80, a43+c81, a43+c82, a43+c83, a43+c84, a43+c85, a43+c86, a43+c87, a43+c88, a43+c89, a43+c90, a43+c91, a43+c92, a43+c93, a43+c94, a43+c95, a43+c96, a43+c97, a43+c98, a43+c99, a43+c100, a43+c101, a43+c102, a43+c103, a43+c104, a43+c105, a43+c106, a43+c107, a43+c108, a43+c109, a43+c110, a43+c111, a43+c112, a43+c113, a43+c114, a43+c115, a43+c116, a43+c117, a43+c118, a43+c119, a43+c120, a43+c121, a43+c122, a43+c123, a43+c124, a43+c125, a43+c126, a43+c127, a43+c128, a43+c129, a43+c130, a43+c131, a43+c132, a43+c133, a43+c134, a43+c135, a43+c136, a43+c137, a43+c138, a43+c139, a43+c140, a43+c141, a43+c142, a43+c143, a43+c144, a43+c145, a43+c146, a43+c147, a43+c148, a43+c149, a43+c150, a43+c151, a43+c152, a43+c153, a43+c154, a43+c155, a43+c156, a43+c157, a43+c158, a43+c159, a43+c160, a43+c161, a43+c162, a43+c163, a43+c164, a43+c165, a43+c166, a43+c167, a43+c168, a43+c169, a43+c170, a43+c171, a43+c172, a43+c173, a43+c174, a43+c175, a43+c176, a43+c177, a43+c178, a43+c179, a43+c180, a43+c181, a43+c182, a43+c183, a43+c184, a43+c185, a43+c186, a43+c187, a43+c188, a43+c189, a43+c190, a43+c191, a43+c192, a43+c193, a43+c194, a43+c195, a43+c196, a43+c197, a43+c198, a43+c199, a43+c200, a43+c201, a43+c202, a43+c203, a43+c204, a43+c205, a43+c206, a43+c207, a43+c208, a43+c209, a43+c210, a43+c211, a44+c1, a44+c2, a44+c3, a44+c4, a44+c5, a44+c6, a44+c7, a44+c8, a44+c9, a44+c10, a44+c11, a44+c12, a44+c13, a44+c14, a44+c15, a44+c16, a44+c17, a44+c18, a44+c19, a44+c20, a44+c21, a44+c22, a44+c23, a44+c24, a44+c25, a44+c26, a44+c27, a44+c28, a44+c29, a44+c30, a44+c31, a44+c32, a44+c33, a44+c34, a44+c35, a44+c36, a44+c37, a44+c38, a44+c39, a44+c40, a44+c41, a44+c42, a44+c43, a44+c44, a44+c45, a44+c46, a44+c47, a44+c48, a44+c49, a44+c50, a44+c51, a44+c52, a44+c53, a44+c54, a44+c55, a44+c56, a44+c57, a44+c58, a44+c59, a44+c60, a44+c61, a44+c62, a44+c63, a44+c64, a44+c65, a44+c66, a44+c67, a44+c68, a44+c69, a44+c70, a44+c71, a44+c72, a44+c73, a44+c74, a44+c75, a44+c76, a44+c77, a44+c78, a44+c79, a44+c80, a44+c81, a44+c82, a44+c83, a44+c84, a44+c85, a44+c86, a44+c87, a44+c88, a44+c89, a44+c90, a44+c91, a44+c92, a44+c93, a44+c94, a44+c95, a44+c96, a44+c97, a44+c98, a44+c99, a44+c100, a44+c101, a44+c102, a44+c103, a44+c104, a44+c105, a44+c106, a44+c107, a44+c108, a44+c109, a44+c110, a44+c111, a44+c112, a44+c113, a44+c114, a44+c115, a44+c116, a44+c117, a44+c118, a44+c119, a44+c120, a44+c121, a44+c122, a44+c123, a44+c124, a44+c125, a44+c126, a44+c127, a44+c128, a44+c129, a44+c130, a44+c131, a44+c132, a44+c133, a44+c134, a44+c135, a44+c136, a44+c137, a44+c138, a44+c139, a44+c140, a44+c141, a44+c142, a44+c143, a44+c144, a44+c145, a44+c146, a44+c147, a44+c148, a44+c149, a44+c150, a44+c151, a44+c152, a44+c153, a44+c154, a44+c155, a44+c156, a44+c157, a44+c158, a44+c159, a44+c160, a44+c161, a44+c162, a44+c163, a44+c164, a44+c165, a44+c166, a44+c167, a44+c168, a44+c169, a44+c170, a44+c171, a44+c172, a44+c173, a44+c174, a44+c175, a44+c176, a44+c177, a44+c178, a44+c179, a44+c180, a44+c181, a44+c182, a44+c183, a44+c184, a44+c185, a44+c186, a44+c187, a44+c188, a44+c189, a44+c190, a44+c191, a44+c192, a44+c193, a44+c194, a44+c195, a44+c196, a44+c197, a44+c198, a44+c199, a44+c200, a44+c201, a44+c202, a44+c203, a44+c204, a44+c205, a44+c206, a44+c207, a44+c208, a44+c209, a44+c210, a44+c211, a45+c1, a45+c2, a45+c3, a45+c4, a45+c5, a45+c6, a45+c7, a45+c8, a45+c9, a45+c10, a45+c11, a45+c12, a45+c13, a45+c14, a45+c15, a45+c16, a45+c17, a45+c18, a45+c19, a45+c20, a45+c21, a45+c22, a45+c23, a45+c24, a45+c25, a45+c26, a45+c27, a45+c28, a45+c29, a45+c30, a45+c31, a45+c32, a45+c33, a45+c34, a45+c35, a45+c36, a45+c37, a45+c38, a45+c39, a45+c40, a45+c41, a45+c42, a45+c43, a45+c44, a45+c45, a45+c46, a45+c47, a45+c48, a45+c49, a45+c50, a45+c51, a45+c52, a45+c53, a45+c54, a45+c55, a45+c56, a45+c57, a45+c58, a45+c59, a45+c60, a45+c61, a45+c62, a45+c63, a45+c64, a45+c65, a45+c66, a45+c67, a45+c68, a45+c69, a45+c70, a45+c71, a45+c72, a45+c73, a45+c74, a45+c75, a45+c76, a45+c77, a45+c78, a45+c79, a45+c80, a45+c81, a45+c82, a45+c83, a45+c84, a45+c85, a45+c86, a45+c87, a45+c88, a45+c89, a45+c90, a45+c91, a45+c92, a45+c93, a45+c94, a45+c95, a45+c96, a45+c97, a45+c98, a45+c99, a45+c100, a45+c101, a45+c102, a45+c103, a45+c104, a45+c105, a45+c106, a45+c107, a45+c108, a45+c109, a45+c110, a45+c111, a45+c112, a45+c113, a45+c114, a45+c115, a45+c116, a45+c117, a45+c118, a45+c119, a45+c120, a45+c121, a45+c122, a45+c123, a45+c124, a45+c125, a45+c126, a45+c127, a45+c128, a45+c129, a45+c130, a45+c131, a45+c132, a45+c133, a45+c134, a45+c135, a45+c136, a45+c137, a45+c138, a45+c139, a45+c140, a45+c141, a45+c142, a45+c143, a45+c144, a45+c145, a45+c146, a45+c147, a45+c148, a45+c149, a45+c150, a45+c151, a45+c152, a45+c153, a45+c154, a45+c155, a45+c156, a45+c157, a45+c158, a45+c159, a45+c160, a45+c161, a45+c162, a45+c163, a45+c164, a45+c165, a45+c166, a45+c167, a45+c168, a45+c169, a45+c170, a45+c171, a45+c172, a45+c173, a45+c174, a45+c175, a45+c176, a45+c177, a45+c178, a45+c179, a45+c180, a45+c181, a45+c182, a45+c183, a45+c184, a45+c185, a45+c186, a45+c187, a45+c188, a45+c189, a45+c190, a45+c191, a45+c192, a45+c193, a45+c194, a45+c195, a45+c196, a45+c197, a45+c198, a45+c199, a45+c200, a45+c201, a45+c202, a45+c203, a45+c204, a45+c205, a45+c206, a45+c207, a45+c208, a45+c209, a45+c210, a45+c211, a46+c1, a46+c2, a46+c3, a46+c4, a46+c5, a46+c6, a46+c7, a46+c8, a46+c9, a46+c10, a46+c11, a46+c12, a46+c13, a46+c14, a46+c15, a46+c16, a46+c17, a46+c18, a46+c19, a46+c20, a46+c21, a46+c22, a46+c23, a46+c24, a46+c25, a46+c26, a46+c27, a46+c28, a46+c29, a46+c30, a46+c31, a46+c32, a46+c33, a46+c34, a46+c35, a46+c36, a46+c37, a46+c38, a46+c39, a46+c40, a46+c41, a46+c42, a46+c43, a46+c44, a46+c45, a46+c46, a46+c47, a46+c48, a46+c49, a46+c50, a46+c51, a46+c52, a46+c53, a46+c54, a46+c55, a46+c56, a46+c57, a46+c58, a46+c59, a46+c60, a46+c61, a46+c62, a46+c63, a46+c64, a46+c65, a46+c66, a46+c67, a46+c68, a46+c69, a46+c70, a46+c71, a46+c72, a46+c73, a46+c74, a46+c75, a46+c76, a46+c77, a46+c78, a46+c79, a46+c80, a46+c81, a46+c82, a46+c83, a46+c84, a46+c85, a46+c86, a46+c87, a46+c88, a46+c89, a46+c90, a46+c91, a46+c92, a46+c93, a46+c94, a46+c95, a46+c96, a46+c97, a46+c98, a46+c99, a46+c100, a46+c101, a46+c102, a46+c103, a46+c104, a46+c105, a46+c106, a46+c107, a46+c108, a46+c109, a46+c110, a46+c111, a46+c112, a46+c113, a46+c114, a46+c115, a46+c116, a46+c117, a46+c118, a46+c119, a46+c120, a46+c121, a46+c122, a46+c123, a46+c124, a46+c125, a46+c126, a46+c127, a46+c128, a46+c129, a46+c130, a46+c131, a46+c132, a46+c133, a46+c134, a46+c135, a46+c136, a46+c137, a46+c138, a46+c139, a46+c140, a46+c141, a46+c142, a46+c143, a46+c144, a46+c145, a46+c146, a46+c147, a46+c148, a46+c149, a46+c150, a46+c151, a46+c152, a46+c153, a46+c154, a46+c155, a46+c156, a46+c157, a46+c158, a46+c159, a46+c160, a46+c161, a46+c162, a46+c163, a46+c164, a46+c165, a46+c166, a46+c167, a46+c168, a46+c169, a46+c170, a46+c171, a46+c172, a46+c173, a46+c174, a46+c175, a46+c176, a46+c177, a46+c178, a46+c179, a46+c180, a46+c181, a46+c182, a46+c183, a46+c184, a46+c185, a46+c186, a46+c187, a46+c188, a46+c189, a46+c190, a46+c191, a46+c192, a46+c193, a46+c194, a46+c195, a46+c196, a46+c197, a46+c198, a46+c199, a46+c200, a46+c201, a46+c202, a46+c203, a46+c204, a46+c205, a46+c206, a46+c207, a46+c208, a46+c209, a46+c210, a46+c211, a47+c1, a47+c2, a47+c3, a47+c4, a47+c5, a47+c6, a47+c7, a47+c8, a47+c9, a47+c10, a47+c11, a47+c12, a47+c13, a47+c14, a47+c15, a47+c16, a47+c17, a47+c18, a47+c19, a47+c20, a47+c21, a47+c22, a47+c23, a47+c24, a47+c25, a47+c26, a47+c27, a47+c28, a47+c29, a47+c30, a47+c31, a47+c32, a47+c33, a47+c34, a47+c35, a47+c36, a47+c37, a47+c38, a47+c39, a47+c40, a47+c41, a47+c42, a47+c43, a47+c44, a47+c45, a47+c46, a47+c47, a47+c48, a47+c49, a47+c50, a47+c51, a47+c52, a47+c53, a47+c54, a47+c55, a47+c56, a47+c57, a47+c58, a47+c59, a47+c60, a47+c61, a47+c62, a47+c63, a47+c64, a47+c65, a47+c66, a47+c67, a47+c68, a47+c69, a47+c70, a47+c71, a47+c72, a47+c73, a47+c74, a47+c75, a47+c76, a47+c77, a47+c78, a47+c79, a47+c80, a47+c81, a47+c82, a47+c83, a47+c84, a47+c85, a47+c86, a47+c87, a47+c88, a47+c89, a47+c90, a47+c91, a47+c92, a47+c93, a47+c94, a47+c95, a47+c96, a47+c97, a47+c98, a47+c99, a47+c100, a47+c101, a47+c102, a47+c103, a47+c104, a47+c105, a47+c106, a47+c107, a47+c108, a47+c109, a47+c110, a47+c111, a47+c112, a47+c113, a47+c114, a47+c115, a47+c116, a47+c117, a47+c118, a47+c119, a47+c120, a47+c121, a47+c122, a47+c123, a47+c124, a47+c125, a47+c126, a47+c127, a47+c128, a47+c129, a47+c130, a47+c131, a47+c132, a47+c133, a47+c134, a47+c135, a47+c136, a47+c137, a47+c138, a47+c139, a47+c140, a47+c141, a47+c142, a47+c143, a47+c144, a47+c145, a47+c146, a47+c147, a47+c148, a47+c149, a47+c150, a47+c151, a47+c152, a47+c153, a47+c154, a47+c155, a47+c156, a47+c157, a47+c158, a47+c159, a47+c160, a47+c161, a47+c162, a47+c163, a47+c164, a47+c165, a47+c166, a47+c167, a47+c168, a47+c169, a47+c170, a47+c171, a47+c172, a47+c173, a47+c174, a47+c175, a47+c176, a47+c177, a47+c178, a47+c179, a47+c180, a47+c181, a47+c182, a47+c183, a47+c184, a47+c185, a47+c186, a47+c187, a47+c188, a47+c189, a47+c190, a47+c191, a47+c192, a47+c193, a47+c194, a47+c195, a47+c196, a47+c197, a47+c198, a47+c199, a47+c200, a47+c201, a47+c202, a47+c203, a47+c204, a47+c205, a47+c206, a47+c207, a47+c208, a47+c209, a47+c210, a47+c211, a48+c1, a48+c2, a48+c3, a48+c4, a48+c5, a48+c6, a48+c7, a48+c8, a48+c9, a48+c10, a48+c11, a48+c12, a48+c13, a48+c14, a48+c15, a48+c16, a48+c17, a48+c18, a48+c19, a48+c20, a48+c21, a48+c22, a48+c23, a48+c24, a48+c25, a48+c26, a48+c27, a48+c28, a48+c29, a48+c30, a48+c31, a48+c32, a48+c33, a48+c34, a48+c35, a48+c36, a48+c37, a48+c38, a48+c39, a48+c40, a48+c41, a48+c42, a48+c43, a48+c44, a48+c45, a48+c46, a48+c47, a48+c48, a48+c49, a48+c50, a48+c51, a48+c52, a48+c53, a48+c54, a48+c55, a48+c56, a48+c57, a48+c58, a48+c59, a48+c60, a48+c61, a48+c62, a48+c63, a48+c64, a48+c65, a48+c66, a48+c67, a48+c68, a48+c69, a48+c70, a48+c71, a48+c72, a48+c73, a48+c74, a48+c75, a48+c76, a48+c77, a48+c78, a48+c79, a48+c80, a48+c81, a48+c82, a48+c83, a48+c84, a48+c85, a48+c86, a48+c87, a48+c88, a48+c89, a48+c90, a48+c91, a48+c92, a48+c93, a48+c94, a48+c95, a48+c96, a48+c97, a48+c98, a48+c99, a48+c100, a48+c101, a48+c102, a48+c103, a48+c104, a48+c105, a48+c106, a48+c107, a48+c108, a48+c109, a48+c110, a48+c111, a48+c112, a48+c113, a48+c114, a48+c115, a48+c116, a48+c117, a48+c118, a48+c119, a48+c120, a48+c121, a48+c122, a48+c123, a48+c124, a48+c125, a48+c126, a48+c127, a48+c128, a48+c129, a48+c130, a48+c131, a48+c132, a48+c133, a48+c134, a48+c135, a48+c136, a48+c137, a48+c138, a48+c139, a48+c140, a48+c141, a48+c142, a48+c143, a48+c144, a48+c145, a48+c146, a48+c147, a48+c148, a48+c149, a48+c150, a48+c151, a48+c152, a48+c153, a48+c154, a48+c155, a48+c156, a48+c157, a48+c158, a48+c159, a48+c160, a48+c161, a48+c162, a48+c163, a48+c164, a48+c165, a48+c166, a48+c167, a48+c168, a48+c169, a48+c170, a48+c171, a48+c172, a48+c173, a48+c174, a48+c175, a48+c176, a48+c177, a48+c178, a48+c179, a48+c180, a48+c181, a48+c182, a48+c183, a48+c184, a48+c185, a48+c186, a48+c187, a48+c188, a48+c189, a48+c190, a48+c191, a48+c192, a48+c193, a48+c194, a48+c195, a48+c196, a48+c197, a48+c198, a48+c199, a48+c200, a48+c201, a48+c202, a48+c203, a48+c204, a48+c205, a48+c206, a48+c207, a48+c208, a48+c209, a48+c210, a48+c211, a49+c1, a49+c2, a49+c3, a49+c4, a49+c5, a49+c6, a49+c7, a49+c8, a49+c9, a49+c10, a49+c11, a49+c12, a49+c13, a49+c14, a49+c15, a49+c16, a49+c17, a49+c18, a49+c19, a49+c20, a49+c21, a49+c22, a49+c23, a49+c24, a49+c25, a49+c26, a49+c27, a49+c28, a49+c29, a49+c30, a49+c31, a49+c32, a49+c33, a49+c34, a49+c35, a49+c36, a49+c37, a49+c38, a49+c39, a49+c40, a49+c41, a49+c42, a49+c43, a49+c44, a49+c45, a49+c46, a49+c47, a49+c48, a49+c49, a49+c50, a49+c51, a49+c52, a49+c53, a49+c54, a49+c55, a49+c56, a49+c57, a49+c58, a49+c59, a49+c60, a49+c61, a49+c62, a49+c63, a49+c64, a49+c65, a49+c66, a49+c67, a49+c68, a49+c69, a49+c70, a49+c71, a49+c72, a49+c73, a49+c74, a49+c75, a49+c76, a49+c77, a49+c78, a49+c79, a49+c80, a49+c81, a49+c82, a49+c83, a49+c84, a49+c85, a49+c86, a49+c87, a49+c88, a49+c89, a49+c90, a49+c91, a49+c92, a49+c93, a49+c94, a49+c95, a49+c96, a49+c97, a49+c98, a49+c99, a49+c100, a49+c101, a49+c102, a49+c103, a49+c104, a49+c105, a49+c106, a49+c107, a49+c108, a49+c109, a49+c110, a49+c111, a49+c112, a49+c113, a49+c114, a49+c115, a49+c116, a49+c117, a49+c118, a49+c119, a49+c120, a49+c121, a49+c122, a49+c123, a49+c124, a49+c125, a49+c126, a49+c127, a49+c128, a49+c129, a49+c130, a49+c131, a49+c132, a49+c133, a49+c134, a49+c135, a49+c136, a49+c137, a49+c138, a49+c139, a49+c140, a49+c141, a49+c142, a49+c143, a49+c144, a49+c145, a49+c146, a49+c147, a49+c148, a49+c149, a49+c150, a49+c151, a49+c152, a49+c153, a49+c154, a49+c155, a49+c156, a49+c157, a49+c158, a49+c159, a49+c160, a49+c161, a49+c162, a49+c163, a49+c164, a49+c165, a49+c166, a49+c167, a49+c168, a49+c169, a49+c170, a49+c171, a49+c172, a49+c173, a49+c174, a49+c175, a49+c176, a49+c177, a49+c178, a49+c179, a49+c180, a49+c181, a49+c182, a49+c183, a49+c184, a49+c185, a49+c186, a49+c187, a49+c188, a49+c189, a49+c190, a49+c191, a49+c192, a49+c193, a49+c194, a49+c195, a49+c196, a49+c197, a49+c198, a49+c199, a49+c200, a49+c201, a49+c202, a49+c203, a49+c204, a49+c205, a49+c206, a49+c207, a49+c208, a49+c209, a49+c210, a49+c211, a50+c1, a50+c2, a50+c3, a50+c4, a50+c5, a50+c6, a50+c7, a50+c8, a50+c9, a50+c10, a50+c11, a50+c12, a50+c13, a50+c14, a50+c15, a50+c16, a50+c17, a50+c18, a50+c19, a50+c20, a50+c21, a50+c22, a50+c23, a50+c24, a50+c25, a50+c26, a50+c27, a50+c28, a50+c29, a50+c30, a50+c31, a50+c32, a50+c33, a50+c34, a50+c35, a50+c36, a50+c37, a50+c38, a50+c39, a50+c40, a50+c41, a50+c42, a50+c43, a50+c44, a50+c45, a50+c46, a50+c47, a50+c48, a50+c49, a50+c50, a50+c51, a50+c52, a50+c53, a50+c54, a50+c55, a50+c56, a50+c57, a50+c58, a50+c59, a50+c60, a50+c61, a50+c62, a50+c63, a50+c64, a50+c65, a50+c66, a50+c67, a50+c68, a50+c69, a50+c70, a50+c71, a50+c72, a50+c73, a50+c74, a50+c75, a50+c76, a50+c77, a50+c78, a50+c79, a50+c80, a50+c81, a50+c82, a50+c83, a50+c84, a50+c85, a50+c86, a50+c87, a50+c88, a50+c89, a50+c90, a50+c91, a50+c92, a50+c93, a50+c94, a50+c95, a50+c96, a50+c97, a50+c98, a50+c99, a50+c100, a50+c101, a50+c102, a50+c103, a50+c104, a50+c105, a50+c106, a50+c107, a50+c108, a50+c109, a50+c110, a50+c111, a50+c112, a50+c113, a50+c114, a50+c115, a50+c116, a50+c117, a50+c118, a50+c119, a50+c120, a50+c121, a50+c122, a50+c123, a50+c124, a50+c125, a50+c126, a50+c127, a50+c128, a50+c129, a50+c130, a50+c131, a50+c132, a50+c133, a50+c134, a50+c135, a50+c136, a50+c137, a50+c138, a50+c139, a50+c140, a50+c141, a50+c142, a50+c143, a50+c144, a50+c145, a50+c146, a50+c147, a50+c148, a50+c149, a50+c150, a50+c151, a50+c152, a50+c153, a50+c154, a50+c155, a50+c156, a50+c157, a50+c158, a50+c159, a50+c160, a50+c161, a50+c162, a50+c163, a50+c164, a50+c165, a50+c166, a50+c167, a50+c168, a50+c169, a50+c170, a50+c171, a50+c172, a50+c173, a50+c174, a50+c175, a50+c176, a50+c177, a50+c178, a50+c179, a50+c180, a50+c181, a50+c182, a50+c183, a50+c184, a50+c185, a50+c186, a50+c187, a50+c188, a50+c189, a50+c190, a50+c191, a50+c192, a50+c193, a50+c194, a50+c195, a50+c196, a50+c197, a50+c198, a50+c199, a50+c200, a50+c201, a50+c202, a50+c203, a50+c204, a50+c205, a50+c206, a50+c207, a50+c208, a50+c209, a50+c210, a50+c211, a51+c1, a51+c2, a51+c3, a51+c4, a51+c5, a51+c6, a51+c7, a51+c8, a51+c9, a51+c10, a51+c11, a51+c12, a51+c13, a51+c14, a51+c15, a51+c16, a51+c17, a51+c18, a51+c19, a51+c20, a51+c21, a51+c22, a51+c23, a51+c24, a51+c25, a51+c26, a51+c27, a51+c28, a51+c29, a51+c30, a51+c31, a51+c32, a51+c33, a51+c34, a51+c35, a51+c36, a51+c37, a51+c38, a51+c39, a51+c40, a51+c41, a51+c42, a51+c43, a51+c44, a51+c45, a51+c46, a51+c47, a51+c48, a51+c49, a51+c50, a51+c51, a51+c52, a51+c53, a51+c54, a51+c55, a51+c56, a51+c57, a51+c58, a51+c59, a51+c60, a51+c61, a51+c62, a51+c63, a51+c64, a51+c65, a51+c66, a51+c67, a51+c68, a51+c69, a51+c70, a51+c71, a51+c72, a51+c73, a51+c74, a51+c75, a51+c76, a51+c77, a51+c78, a51+c79, a51+c80, a51+c81, a51+c82, a51+c83, a51+c84, a51+c85, a51+c86, a51+c87, a51+c88, a51+c89, a51+c90, a51+c91, a51+c92, a51+c93, a51+c94, a51+c95, a51+c96, a51+c97, a51+c98, a51+c99, a51+c100, a51+c101, a51+c102, a51+c103, a51+c104, a51+c105, a51+c106, a51+c107, a51+c108, a51+c109, a51+c110, a51+c111, a51+c112, a51+c113, a51+c114, a51+c115, a51+c116, a51+c117, a51+c118, a51+c119, a51+c120, a51+c121, a51+c122, a51+c123, a51+c124, a51+c125, a51+c126, a51+c127, a51+c128, a51+c129, a51+c130, a51+c131, a51+c132, a51+c133, a51+c134, a51+c135, a51+c136, a51+c137, a51+c138, a51+c139, a51+c140, a51+c141, a51+c142, a51+c143, a51+c144, a51+c145, a51+c146, a51+c147, a51+c148, a51+c149, a51+c150, a51+c151, a51+c152, a51+c153, a51+c154, a51+c155, a51+c156, a51+c157, a51+c158, a51+c159, a51+c160, a51+c161, a51+c162, a51+c163, a51+c164, a51+c165, a51+c166, a51+c167, a51+c168, a51+c169, a51+c170, a51+c171, a51+c172, a51+c173, a51+c174, a51+c175, a51+c176, a51+c177, a51+c178, a51+c179, a51+c180, a51+c181, a51+c182, a51+c183, a51+c184, a51+c185, a51+c186, a51+c187, a51+c188, a51+c189, a51+c190, a51+c191, a51+c192, a51+c193, a51+c194, a51+c195, a51+c196, a51+c197, a51+c198, a51+c199, a51+c200, a51+c201, a51+c202, a51+c203, a51+c204, a51+c205, a51+c206, a51+c207, a51+c208, a51+c209, a51+c210, a51+c211, a52+c1, a52+c2, a52+c3, a52+c4, a52+c5, a52+c6, a52+c7, a52+c8, a52+c9, a52+c10, a52+c11, a52+c12, a52+c13, a52+c14, a52+c15, a52+c16, a52+c17, a52+c18, a52+c19, a52+c20, a52+c21, a52+c22, a52+c23, a52+c24, a52+c25, a52+c26, a52+c27, a52+c28, a52+c29, a52+c30, a52+c31, a52+c32, a52+c33, a52+c34, a52+c35, a52+c36, a52+c37, a52+c38, a52+c39, a52+c40, a52+c41, a52+c42, a52+c43, a52+c44, a52+c45, a52+c46, a52+c47, a52+c48, a52+c49, a52+c50, a52+c51, a52+c52, a52+c53, a52+c54, a52+c55, a52+c56, a52+c57, a52+c58, a52+c59, a52+c60, a52+c61, a52+c62, a52+c63, a52+c64, a52+c65, a52+c66, a52+c67, a52+c68, a52+c69, a52+c70, a52+c71, a52+c72, a52+c73, a52+c74, a52+c75, a52+c76, a52+c77, a52+c78, a52+c79, a52+c80, a52+c81, a52+c82, a52+c83, a52+c84, a52+c85, a52+c86, a52+c87, a52+c88, a52+c89, a52+c90, a52+c91, a52+c92, a52+c93, a52+c94, a52+c95, a52+c96, a52+c97, a52+c98, a52+c99, a52+c100, a52+c101, a52+c102, a52+c103, a52+c104, a52+c105, a52+c106, a52+c107, a52+c108, a52+c109, a52+c110, a52+c111, a52+c112, a52+c113, a52+c114, a52+c115, a52+c116, a52+c117, a52+c118, a52+c119, a52+c120, a52+c121, a52+c122, a52+c123, a52+c124, a52+c125, a52+c126, a52+c127, a52+c128, a52+c129, a52+c130, a52+c131, a52+c132, a52+c133, a52+c134, a52+c135, a52+c136, a52+c137, a52+c138, a52+c139, a52+c140, a52+c141, a52+c142, a52+c143, a52+c144, a52+c145, a52+c146, a52+c147, a52+c148, a52+c149, a52+c150, a52+c151, a52+c152, a52+c153, a52+c154, a52+c155, a52+c156, a52+c157, a52+c158, a52+c159, a52+c160, a52+c161, a52+c162, a52+c163, a52+c164, a52+c165, a52+c166, a52+c167, a52+c168, a52+c169, a52+c170, a52+c171, a52+c172, a52+c173, a52+c174, a52+c175, a52+c176, a52+c177, a52+c178, a52+c179, a52+c180, a52+c181, a52+c182, a52+c183, a52+c184, a52+c185, a52+c186, a52+c187, a52+c188, a52+c189, a52+c190, a52+c191, a52+c192, a52+c193, a52+c194, a52+c195, a52+c196, a52+c197, a52+c198, a52+c199, a52+c200, a52+c201, a52+c202, a52+c203, a52+c204, a52+c205, a52+c206, a52+c207, a52+c208, a52+c209, a52+c210, a52+c211, a53+c1, a53+c2, a53+c3, a53+c4, a53+c5, a53+c6, a53+c7, a53+c8, a53+c9, a53+c10, a53+c11, a53+c12, a53+c13, a53+c14, a53+c15, a53+c16, a53+c17, a53+c18, a53+c19, a53+c20, a53+c21, a53+c22, a53+c23, a53+c24, a53+c25, a53+c26, a53+c27, a53+c28, a53+c29, a53+c30, a53+c31, a53+c32, a53+c33, a53+c34, a53+c35, a53+c36, a53+c37, a53+c38, a53+c39, a53+c40, a53+c41, a53+c42, a53+c43, a53+c44, a53+c45, a53+c46, a53+c47, a53+c48, a53+c49, a53+c50, a53+c51, a53+c52, a53+c53, a53+c54, a53+c55, a53+c56, a53+c57, a53+c58, a53+c59, a53+c60, a53+c61, a53+c62, a53+c63, a53+c64, a53+c65, a53+c66, a53+c67, a53+c68, a53+c69, a53+c70, a53+c71, a53+c72, a53+c73, a53+c74, a53+c75, a53+c76, a53+c77, a53+c78, a53+c79, a53+c80, a53+c81, a53+c82, a53+c83, a53+c84, a53+c85, a53+c86, a53+c87, a53+c88, a53+c89, a53+c90, a53+c91, a53+c92, a53+c93, a53+c94, a53+c95, a53+c96, a53+c97, a53+c98, a53+c99, a53+c100, a53+c101, a53+c102, a53+c103, a53+c104, a53+c105, a53+c106, a53+c107, a53+c108, a53+c109, a53+c110, a53+c111, a53+c112, a53+c113, a53+c114, a53+c115, a53+c116, a53+c117, a53+c118, a53+c119, a53+c120, a53+c121, a53+c122, a53+c123, a53+c124, a53+c125, a53+c126, a53+c127, a53+c128, a53+c129, a53+c130, a53+c131, a53+c132, a53+c133, a53+c134, a53+c135, a53+c136, a53+c137, a53+c138, a53+c139, a53+c140, a53+c141, a53+c142, a53+c143, a53+c144, a53+c145, a53+c146, a53+c147, a53+c148, a53+c149, a53+c150, a53+c151, a53+c152, a53+c153, a53+c154, a53+c155, a53+c156, a53+c157, a53+c158, a53+c159, a53+c160, a53+c161, a53+c162, a53+c163, a53+c164, a53+c165, a53+c166, a53+c167, a53+c168, a53+c169, a53+c170, a53+c171, a53+c172, a53+c173, a53+c174, a53+c175, a53+c176, a53+c177, a53+c178, a53+c179, a53+c180, a53+c181, a53+c182, a53+c183, a53+c184, a53+c185, a53+c186, a53+c187, a53+c188, a53+c189, a53+c190, a53+c191, a53+c192, a53+c193, a53+c194, a53+c195, a53+c196, a53+c197, a53+c198, a53+c199, a53+c200, a53+c201, a53+c202, a53+c203, a53+c204, a53+c205, a53+c206, a53+c207, a53+c208, a53+c209, a53+c210, a53+c211, a54+c1, a54+c2, a54+c3, a54+c4, a54+c5, a54+c6, a54+c7, a54+c8, a54+c9, a54+c10, a54+c11, a54+c12, a54+c13, a54+c14, a54+c15, a54+c16, a54+c17, a54+c18, a54+c19, a54+c20, a54+c21, a54+c22, a54+c23, a54+c24, a54+c25, a54+c26, a54+c27, a54+c28, a54+c29, a54+c30, a54+c31, a54+c32, a54+c33, a54+c34, a54+c35, a54+c36, a54+c37, a54+c38, a54+c39, a54+c40, a54+c41, a54+c42, a54+c43, a54+c44, a54+c45, a54+c46, a54+c47, a54+c48, a54+c49, a54+c50, a54+c51, a54+c52, a54+c53, a54+c54, a54+c55, a54+c56, a54+c57, a54+c58, a54+c59, a54+c60, a54+c61, a54+c62, a54+c63, a54+c64, a54+c65, a54+c66, a54+c67, a54+c68, a54+c69, a54+c70, a54+c71, a54+c72, a54+c73, a54+c74, a54+c75, a54+c76, a54+c77, a54+c78, a54+c79, a54+c80, a54+c81, a54+c82, a54+c83, a54+c84, a54+c85, a54+c86, a54+c87, a54+c88, a54+c89, a54+c90, a54+c91, a54+c92, a54+c93, a54+c94, a54+c95, a54+c96, a54+c97, a54+c98, a54+c99, a54+c100, a54+c101, a54+c102, a54+c103, a54+c104, a54+c105, a54+c106, a54+c107, a54+c108, a54+c109, a54+c110, a54+c111, a54+c112, a54+c113, a54+c114, a54+c115, a54+c116, a54+c117, a54+c118, a54+c119, a54+c120, a54+c121, a54+c122, a54+c123, a54+c124, a54+c125, a54+c126, a54+c127, a54+c128, a54+c129, a54+c130, a54+c131, a54+c132, a54+c133, a54+c134, a54+c135, a54+c136, a54+c137, a54+c138, a54+c139, a54+c140, a54+c141, a54+c142, a54+c143, a54+c144, a54+c145, a54+c146, a54+c147, a54+c148, a54+c149, a54+c150, a54+c151, a54+c152, a54+c153, a54+c154, a54+c155, a54+c156, a54+c157, a54+c158, a54+c159, a54+c160, a54+c161, a54+c162, a54+c163, a54+c164, a54+c165, a54+c166, a54+c167, a54+c168, a54+c169, a54+c170, a54+c171, a54+c172, a54+c173, a54+c174, a54+c175, a54+c176, a54+c177, a54+c178, a54+c179, a54+c180, a54+c181, a54+c182, a54+c183, a54+c184, a54+c185, a54+c186, a54+c187, a54+c188, a54+c189, a54+c190, a54+c191, a54+c192, a54+c193, a54+c194, a54+c195, a54+c196, a54+c197, a54+c198, a54+c199, a54+c200, a54+c201, a54+c202, a54+c203, a54+c204, a54+c205, a54+c206, a54+c207, a54+c208, a54+c209, a54+c210, a54+c211, a55+c1, a55+c2, a55+c3, a55+c4, a55+c5, a55+c6, a55+c7, a55+c8, a55+c9, a55+c10, a55+c11, a55+c12, a55+c13, a55+c14, a55+c15, a55+c16, a55+c17, a55+c18, a55+c19, a55+c20, a55+c21, a55+c22, a55+c23, a55+c24, a55+c25, a55+c26, a55+c27, a55+c28, a55+c29, a55+c30, a55+c31, a55+c32, a55+c33, a55+c34, a55+c35, a55+c36, a55+c37, a55+c38, a55+c39, a55+c40, a55+c41, a55+c42, a55+c43, a55+c44, a55+c45, a55+c46, a55+c47, a55+c48, a55+c49, a55+c50, a55+c51, a55+c52, a55+c53, a55+c54, a55+c55, a55+c56, a55+c57, a55+c58, a55+c59, a55+c60, a55+c61, a55+c62, a55+c63, a55+c64, a55+c65, a55+c66, a55+c67, a55+c68, a55+c69, a55+c70, a55+c71, a55+c72, a55+c73, a55+c74, a55+c75, a55+c76, a55+c77, a55+c78, a55+c79, a55+c80, a55+c81, a55+c82, a55+c83, a55+c84, a55+c85, a55+c86, a55+c87, a55+c88, a55+c89, a55+c90, a55+c91, a55+c92, a55+c93, a55+c94, a55+c95, a55+c96, a55+c97, a55+c98, a55+c99, a55+c100, a55+c101, a55+c102, a55+c103, a55+c104, a55+c105, a55+c106, a55+c107, a55+c108, a55+c109, a55+c110, a55+c111, a55+c112, a55+c113, a55+c114, a55+c115, a55+c116, a55+c117, a55+c118, a55+c119, a55+c120, a55+c121, a55+c122, a55+c123, a55+c124, a55+c125, a55+c126, a55+c127, a55+c128, a55+c129, a55+c130, a55+c131, a55+c132, a55+c133, a55+c134, a55+c135, a55+c136, a55+c137, a55+c138, a55+c139, a55+c140, a55+c141, a55+c142, a55+c143, a55+c144, a55+c145, a55+c146, a55+c147, a55+c148, a55+c149, a55+c150, a55+c151, a55+c152, a55+c153, a55+c154, a55+c155, a55+c156, a55+c157, a55+c158, a55+c159, a55+c160, a55+c161, a55+c162, a55+c163, a55+c164, a55+c165, a55+c166, a55+c167, a55+c168, a55+c169, a55+c170, a55+c171, a55+c172, a55+c173, a55+c174, a55+c175, a55+c176, a55+c177, a55+c178, a55+c179, a55+c180, a55+c181, a55+c182, a55+c183, a55+c184, a55+c185, a55+c186, a55+c187, a55+c188, a55+c189, a55+c190, a55+c191, a55+c192, a55+c193, a55+c194, a55+c195, a55+c196, a55+c197, a55+c198, a55+c199, a55+c200, a55+c201, a55+c202, a55+c203, a55+c204, a55+c205, a55+c206, a55+c207, a55+c208, a55+c209, a55+c210, a55+c211, a56+c1, a56+c2, a56+c3, a56+c4, a56+c5, a56+c6, a56+c7, a56+c8, a56+c9, a56+c10, a56+c11, a56+c12, a56+c13, a56+c14, a56+c15, a56+c16, a56+c17, a56+c18, a56+c19, a56+c20, a56+c21, a56+c22, a56+c23, a56+c24, a56+c25, a56+c26, a56+c27, a56+c28, a56+c29, a56+c30, a56+c31, a56+c32, a56+c33, a56+c34, a56+c35, a56+c36, a56+c37, a56+c38, a56+c39, a56+c40, a56+c41, a56+c42, a56+c43, a56+c44, a56+c45, a56+c46, a56+c47, a56+c48, a56+c49, a56+c50, a56+c51, a56+c52, a56+c53, a56+c54, a56+c55, a56+c56, a56+c57, a56+c58, a56+c59, a56+c60, a56+c61, a56+c62, a56+c63, a56+c64, a56+c65, a56+c66, a56+c67, a56+c68, a56+c69, a56+c70, a56+c71, a56+c72, a56+c73, a56+c74, a56+c75, a56+c76, a56+c77, a56+c78, a56+c79, a56+c80, a56+c81, a56+c82, a56+c83, a56+c84, a56+c85, a56+c86, a56+c87, a56+c88, a56+c89, a56+c90, a56+c91, a56+c92, a56+c93, a56+c94, a56+c95, a56+c96, a56+c97, a56+c98, a56+c99, a56+c100, a56+c101, a56+c102, a56+c103, a56+c104, a56+c105, a56+c106, a56+c107, a56+c108, a56+c109, a56+c110, a56+c111, a56+c112, a56+c113, a56+c114, a56+c115, a56+c116, a56+c117, a56+c118, a56+c119, a56+c120, a56+c121, a56+c122, a56+c123, a56+c124, a56+c125, a56+c126, a56+c127, a56+c128, a56+c129, a56+c130, a56+c131, a56+c132, a56+c133, a56+c134, a56+c135, a56+c136, a56+c137, a56+c138, a56+c139, a56+c140, a56+c141, a56+c142, a56+c143, a56+c144, a56+c145, a56+c146, a56+c147, a56+c148, a56+c149, a56+c150, a56+c151, a56+c152, a56+c153, a56+c154, a56+c155, a56+c156, a56+c157, a56+c158, a56+c159, a56+c160, a56+c161, a56+c162, a56+c163, a56+c164, a56+c165, a56+c166, a56+c167, a56+c168, a56+c169, a56+c170, a56+c171, a56+c172, a56+c173, a56+c174, a56+c175, a56+c176, a56+c177, a56+c178, a56+c179, a56+c180, a56+c181, a56+c182, a56+c183, a56+c184, a56+c185, a56+c186, a56+c187, a56+c188, a56+c189, a56+c190, a56+c191, a56+c192, a56+c193, a56+c194, a56+c195, a56+c196, a56+c197, a56+c198, a56+c199, a56+c200, a56+c201, a56+c202, a56+c203, a56+c204, a56+c205, a56+c206, a56+c207, a56+c208, a56+c209, a56+c210, a56+c211, a57+c1, a57+c2, a57+c3, a57+c4, a57+c5, a57+c6, a57+c7, a57+c8, a57+c9, a57+c10, a57+c11, a57+c12, a57+c13, a57+c14, a57+c15, a57+c16, a57+c17, a57+c18, a57+c19, a57+c20, a57+c21, a57+c22, a57+c23, a57+c24, a57+c25, a57+c26, a57+c27, a57+c28, a57+c29, a57+c30, a57+c31, a57+c32, a57+c33, a57+c34, a57+c35, a57+c36, a57+c37, a57+c38, a57+c39, a57+c40, a57+c41, a57+c42, a57+c43, a57+c44, a57+c45, a57+c46, a57+c47, a57+c48, a57+c49, a57+c50, a57+c51, a57+c52, a57+c53, a57+c54, a57+c55, a57+c56, a57+c57, a57+c58, a57+c59, a57+c60, a57+c61, a57+c62, a57+c63, a57+c64, a57+c65, a57+c66, a57+c67, a57+c68, a57+c69, a57+c70, a57+c71, a57+c72, a57+c73, a57+c74, a57+c75, a57+c76, a57+c77, a57+c78, a57+c79, a57+c80, a57+c81, a57+c82, a57+c83, a57+c84, a57+c85, a57+c86, a57+c87, a57+c88, a57+c89, a57+c90, a57+c91, a57+c92, a57+c93, a57+c94, a57+c95, a57+c96, a57+c97, a57+c98, a57+c99, a57+c100, a57+c101, a57+c102, a57+c103, a57+c104, a57+c105, a57+c106, a57+c107, a57+c108, a57+c109, a57+c110, a57+c111, a57+c112, a57+c113, a57+c114, a57+c115, a57+c116, a57+c117, a57+c118, a57+c119, a57+c120, a57+c121, a57+c122, a57+c123, a57+c124, a57+c125, a57+c126, a57+c127, a57+c128, a57+c129, a57+c130, a57+c131, a57+c132, a57+c133, a57+c134, a57+c135, a57+c136, a57+c137, a57+c138, a57+c139, a57+c140, a57+c141, a57+c142, a57+c143, a57+c144, a57+c145, a57+c146, a57+c147, a57+c148, a57+c149, a57+c150, a57+c151, a57+c152, a57+c153, a57+c154, a57+c155, a57+c156, a57+c157, a57+c158, a57+c159, a57+c160, a57+c161, a57+c162, a57+c163, a57+c164, a57+c165, a57+c166, a57+c167, a57+c168, a57+c169, a57+c170, a57+c171, a57+c172, a57+c173, a57+c174, a57+c175, a57+c176, a57+c177, a57+c178, a57+c179, a57+c180, a57+c181, a57+c182, a57+c183, a57+c184, a57+c185, a57+c186, a57+c187, a57+c188, a57+c189, a57+c190, a57+c191, a57+c192, a57+c193, a57+c194, a57+c195, a57+c196, a57+c197, a57+c198, a57+c199, a57+c200, a57+c201, a57+c202, a57+c203, a57+c204, a57+c205, a57+c206, a57+c207, a57+c208, a57+c209, a57+c210, a57+c211, a58+c1, a58+c2, a58+c3, a58+c4, a58+c5, a58+c6, a58+c7, a58+c8, a58+c9, a58+c10, a58+c11, a58+c12, a58+c13, a58+c14, a58+c15, a58+c16, a58+c17, a58+c18, a58+c19, a58+c20, a58+c21, a58+c22, a58+c23, a58+c24, a58+c25, a58+c26, a58+c27, a58+c28, a58+c29, a58+c30, a58+c31, a58+c32, a58+c33, a58+c34, a58+c35, a58+c36, a58+c37, a58+c38, a58+c39, a58+c40, a58+c41, a58+c42, a58+c43, a58+c44, a58+c45, a58+c46, a58+c47, a58+c48, a58+c49, a58+c50, a58+c51, a58+c52, a58+c53, a58+c54, a58+c55, a58+c56, a58+c57, a58+c58, a58+c59, a58+c60, a58+c61, a58+c62, a58+c63, a58+c64, a58+c65, a58+c66, a58+c67, a58+c68, a58+c69, a58+c70, a58+c71, a58+c72, a58+c73, a58+c74, a58+c75, a58+c76, a58+c77, a58+c78, a58+c79, a58+c80, a58+c81, a58+c82, a58+c83, a58+c84, a58+c85, a58+c86, a58+c87, a58+c88, a58+c89, a58+c90, a58+c91, a58+c92, a58+c93, a58+c94, a58+c95, a58+c96, a58+c97, a58+c98, a58+c99, a58+c100, a58+c101, a58+c102, a58+c103, a58+c104, a58+c105, a58+c106, a58+c107, a58+c108, a58+c109, a58+c110, a58+c111, a58+c112, a58+c113, a58+c114, a58+c115, a58+c116, a58+c117, a58+c118, a58+c119, a58+c120, a58+c121, a58+c122, a58+c123, a58+c124, a58+c125, a58+c126, a58+c127, a58+c128, a58+c129, a58+c130, a58+c131, a58+c132, a58+c133, a58+c134, a58+c135, a58+c136, a58+c137, a58+c138, a58+c139, a58+c140, a58+c141, a58+c142, a58+c143, a58+c144, a58+c145, a58+c146, a58+c147, a58+c148, a58+c149, a58+c150, a58+c151, a58+c152, a58+c153, a58+c154, a58+c155, a58+c156, a58+c157, a58+c158, a58+c159, a58+c160, a58+c161, a58+c162, a58+c163, a58+c164, a58+c165, a58+c166, a58+c167, a58+c168, a58+c169, a58+c170, a58+c171, a58+c172, a58+c173, a58+c174, a58+c175, a58+c176, a58+c177, a58+c178, a58+c179, a58+c180, a58+c181, a58+c182, a58+c183, a58+c184, a58+c185, a58+c186, a58+c187, a58+c188, a58+c189, a58+c190, a58+c191, a58+c192, a58+c193, a58+c194, a58+c195, a58+c196, a58+c197, a58+c198, a58+c199, a58+c200, a58+c201, a58+c202, a58+c203, a58+c204, a58+c205, a58+c206, a58+c207, a58+c208, a58+c209, a58+c210, a58+c211, a59+c1, a59+c2, a59+c3, a59+c4, a59+c5, a59+c6, a59+c7, a59+c8, a59+c9, a59+c10, a59+c11, a59+c12, a59+c13, a59+c14, a59+c15, a59+c16, a59+c17, a59+c18, a59+c19, a59+c20, a59+c21, a59+c22, a59+c23, a59+c24, a59+c25, a59+c26, a59+c27, a59+c28, a59+c29, a59+c30, a59+c31, a59+c32, a59+c33, a59+c34, a59+c35, a59+c36, a59+c37, a59+c38, a59+c39, a59+c40, a59+c41, a59+c42, a59+c43, a59+c44, a59+c45, a59+c46, a59+c47, a59+c48, a59+c49, a59+c50, a59+c51, a59+c52, a59+c53, a59+c54, a59+c55, a59+c56, a59+c57, a59+c58, a59+c59, a59+c60, a59+c61, a59+c62, a59+c63, a59+c64, a59+c65, a59+c66, a59+c67, a59+c68, a59+c69, a59+c70, a59+c71, a59+c72, a59+c73, a59+c74, a59+c75, a59+c76, a59+c77, a59+c78, a59+c79, a59+c80, a59+c81, a59+c82, a59+c83, a59+c84, a59+c85, a59+c86, a59+c87, a59+c88, a59+c89, a59+c90, a59+c91, a59+c92, a59+c93, a59+c94, a59+c95, a59+c96, a59+c97, a59+c98, a59+c99, a59+c100, a59+c101, a59+c102, a59+c103, a59+c104, a59+c105, a59+c106, a59+c107, a59+c108, a59+c109, a59+c110, a59+c111, a59+c112, a59+c113, a59+c114, a59+c115, a59+c116, a59+c117, a59+c118, a59+c119, a59+c120, a59+c121, a59+c122, a59+c123, a59+c124, a59+c125, a59+c126, a59+c127, a59+c128, a59+c129, a59+c130, a59+c131, a59+c132, a59+c133, a59+c134, a59+c135, a59+c136, a59+c137, a59+c138, a59+c139, a59+c140, a59+c141, a59+c142, a59+c143, a59+c144, a59+c145, a59+c146, a59+c147, a59+c148, a59+c149, a59+c150, a59+c151, a59+c152, a59+c153, a59+c154, a59+c155, a59+c156, a59+c157, a59+c158, a59+c159, a59+c160, a59+c161, a59+c162, a59+c163, a59+c164, a59+c165, a59+c166, a59+c167, a59+c168, a59+c169, a59+c170, a59+c171, a59+c172, a59+c173, a59+c174, a59+c175, a59+c176, a59+c177, a59+c178, a59+c179, a59+c180, a59+c181, a59+c182, a59+c183, a59+c184, a59+c185, a59+c186, a59+c187, a59+c188, a59+c189, a59+c190, a59+c191, a59+c192, a59+c193, a59+c194, a59+c195, a59+c196, a59+c197, a59+c198, a59+c199, a59+c200, a59+c201, a59+c202, a59+c203, a59+c204, a59+c205, a59+c206, a59+c207, a59+c208, a59+c209, a59+c210, a59+c211, a60+c1, a60+c2, a60+c3, a60+c4, a60+c5, a60+c6, a60+c7, a60+c8, a60+c9, a60+c10, a60+c11, a60+c12, a60+c13, a60+c14, a60+c15, a60+c16, a60+c17, a60+c18, a60+c19, a60+c20, a60+c21, a60+c22, a60+c23, a60+c24, a60+c25, a60+c26, a60+c27, a60+c28, a60+c29, a60+c30, a60+c31, a60+c32, a60+c33, a60+c34, a60+c35, a60+c36, a60+c37, a60+c38, a60+c39, a60+c40, a60+c41, a60+c42, a60+c43, a60+c44, a60+c45, a60+c46, a60+c47, a60+c48, a60+c49, a60+c50, a60+c51, a60+c52, a60+c53, a60+c54, a60+c55, a60+c56, a60+c57, a60+c58, a60+c59, a60+c60, a60+c61, a60+c62, a60+c63, a60+c64, a60+c65, a60+c66, a60+c67, a60+c68, a60+c69, a60+c70, a60+c71, a60+c72, a60+c73, a60+c74, a60+c75, a60+c76, a60+c77, a60+c78, a60+c79, a60+c80, a60+c81, a60+c82, a60+c83, a60+c84, a60+c85, a60+c86, a60+c87, a60+c88, a60+c89, a60+c90, a60+c91, a60+c92, a60+c93, a60+c94, a60+c95, a60+c96, a60+c97, a60+c98, a60+c99, a60+c100, a60+c101, a60+c102, a60+c103, a60+c104, a60+c105, a60+c106, a60+c107, a60+c108, a60+c109, a60+c110, a60+c111, a60+c112, a60+c113, a60+c114, a60+c115, a60+c116, a60+c117, a60+c118, a60+c119, a60+c120, a60+c121, a60+c122, a60+c123, a60+c124, a60+c125, a60+c126, a60+c127, a60+c128, a60+c129, a60+c130, a60+c131, a60+c132, a60+c133, a60+c134, a60+c135, a60+c136, a60+c137, a60+c138, a60+c139, a60+c140, a60+c141, a60+c142, a60+c143, a60+c144, a60+c145, a60+c146, a60+c147, a60+c148, a60+c149, a60+c150, a60+c151, a60+c152, a60+c153, a60+c154, a60+c155, a60+c156, a60+c157, a60+c158, a60+c159, a60+c160, a60+c161, a60+c162, a60+c163, a60+c164, a60+c165, a60+c166, a60+c167, a60+c168, a60+c169, a60+c170, a60+c171, a60+c172, a60+c173, a60+c174, a60+c175, a60+c176, a60+c177, a60+c178, a60+c179, a60+c180, a60+c181, a60+c182, a60+c183, a60+c184, a60+c185, a60+c186, a60+c187, a60+c188, a60+c189, a60+c190, a60+c191, a60+c192, a60+c193, a60+c194, a60+c195, a60+c196, a60+c197, a60+c198, a60+c199, a60+c200, a60+c201, a60+c202, a60+c203, a60+c204, a60+c205, a60+c206, a60+c207, a60+c208, a60+c209, a60+c210, a60+c211, a61+c1, a61+c2, a61+c3, a61+c4, a61+c5, a61+c6, a61+c7, a61+c8, a61+c9, a61+c10, a61+c11, a61+c12, a61+c13, a61+c14, a61+c15, a61+c16, a61+c17, a61+c18, a61+c19, a61+c20, a61+c21, a61+c22, a61+c23, a61+c24, a61+c25, a61+c26, a61+c27, a61+c28, a61+c29, a61+c30, a61+c31, a61+c32, a61+c33, a61+c34, a61+c35, a61+c36, a61+c37, a61+c38, a61+c39, a61+c40, a61+c41, a61+c42, a61+c43, a61+c44, a61+c45, a61+c46, a61+c47, a61+c48, a61+c49, a61+c50, a61+c51, a61+c52, a61+c53, a61+c54, a61+c55, a61+c56, a61+c57, a61+c58, a61+c59, a61+c60, a61+c61, a61+c62, a61+c63, a61+c64, a61+c65, a61+c66, a61+c67, a61+c68, a61+c69, a61+c70, a61+c71, a61+c72, a61+c73, a61+c74, a61+c75, a61+c76, a61+c77, a61+c78, a61+c79, a61+c80, a61+c81, a61+c82, a61+c83, a61+c84, a61+c85, a61+c86, a61+c87, a61+c88, a61+c89, a61+c90, a61+c91, a61+c92, a61+c93, a61+c94, a61+c95, a61+c96, a61+c97, a61+c98, a61+c99, a61+c100, a61+c101, a61+c102, a61+c103, a61+c104, a61+c105, a61+c106, a61+c107, a61+c108, a61+c109, a61+c110, a61+c111, a61+c112, a61+c113, a61+c114, a61+c115, a61+c116, a61+c117, a61+c118, a61+c119, a61+c120, a61+c121, a61+c122, a61+c123, a61+c124, a61+c125, a61+c126, a61+c127, a61+c128, a61+c129, a61+c130, a61+c131, a61+c132, a61+c133, a61+c134, a61+c135, a61+c136, a61+c137, a61+c138, a61+c139, a61+c140, a61+c141, a61+c142, a61+c143, a61+c144, a61+c145, a61+c146, a61+c147, a61+c148, a61+c149, a61+c150, a61+c151, a61+c152, a61+c153, a61+c154, a61+c155, a61+c156, a61+c157, a61+c158, a61+c159, a61+c160, a61+c161, a61+c162, a61+c163, a61+c164, a61+c165, a61+c166, a61+c167, a61+c168, a61+c169, a61+c170, a61+c171, a61+c172, a61+c173, a61+c174, a61+c175, a61+c176, a61+c177, a61+c178, a61+c179, a61+c180, a61+c181, a61+c182, a61+c183, a61+c184, a61+c185, a61+c186, a61+c187, a61+c188, a61+c189, a61+c190, a61+c191, a61+c192, a61+c193, a61+c194, a61+c195, a61+c196, a61+c197, a61+c198, a61+c199, a61+c200, a61+c201, a61+c202, a61+c203, a61+c204, a61+c205, a61+c206, a61+c207, a61+c208, a61+c209, a61+c210, a61+c211, a62+c1, a62+c2, a62+c3, a62+c4, a62+c5, a62+c6, a62+c7, a62+c8, a62+c9, a62+c10, a62+c11, a62+c12, a62+c13, a62+c14, a62+c15, a62+c16, a62+c17, a62+c18, a62+c19, a62+c20, a62+c21, a62+c22, a62+c23, a62+c24, a62+c25, a62+c26, a62+c27, a62+c28, a62+c29, a62+c30, a62+c31, a62+c32, a62+c33, a62+c34, a62+c35, a62+c36, a62+c37, a62+c38, a62+c39, a62+c40, a62+c41, a62+c42, a62+c43, a62+c44, a62+c45, a62+c46, a62+c47, a62+c48, a62+c49, a62+c50, a62+c51, a62+c52, a62+c53, a62+c54, a62+c55, a62+c56, a62+c57, a62+c58, a62+c59, a62+c60, a62+c61, a62+c62, a62+c63, a62+c64, a62+c65, a62+c66, a62+c67, a62+c68, a62+c69, a62+c70, a62+c71, a62+c72, a62+c73, a62+c74, a62+c75, a62+c76, a62+c77, a62+c78, a62+c79, a62+c80, a62+c81, a62+c82, a62+c83, a62+c84, a62+c85, a62+c86, a62+c87, a62+c88, a62+c89, a62+c90, a62+c91, a62+c92, a62+c93, a62+c94, a62+c95, a62+c96, a62+c97, a62+c98, a62+c99, a62+c100, a62+c101, a62+c102, a62+c103, a62+c104, a62+c105, a62+c106, a62+c107, a62+c108, a62+c109, a62+c110, a62+c111, a62+c112, a62+c113, a62+c114, a62+c115, a62+c116, a62+c117, a62+c118, a62+c119, a62+c120, a62+c121, a62+c122, a62+c123, a62+c124, a62+c125, a62+c126, a62+c127, a62+c128, a62+c129, a62+c130, a62+c131, a62+c132, a62+c133, a62+c134, a62+c135, a62+c136, a62+c137, a62+c138, a62+c139, a62+c140, a62+c141, a62+c142, a62+c143, a62+c144, a62+c145, a62+c146, a62+c147, a62+c148, a62+c149, a62+c150, a62+c151, a62+c152, a62+c153, a62+c154, a62+c155, a62+c156, a62+c157, a62+c158, a62+c159, a62+c160, a62+c161, a62+c162, a62+c163, a62+c164, a62+c165, a62+c166, a62+c167, a62+c168, a62+c169, a62+c170, a62+c171, a62+c172, a62+c173, a62+c174, a62+c175, a62+c176, a62+c177, a62+c178, a62+c179, a62+c180, a62+c181, a62+c182, a62+c183, a62+c184, a62+c185, a62+c186, a62+c187, a62+c188, a62+c189, a62+c190, a62+c191, a62+c192, a62+c193, a62+c194, a62+c195, a62+c196, a62+c197, a62+c198, a62+c199, a62+c200, a62+c201, a62+c202, a62+c203, a62+c204, a62+c205, a62+c206, a62+c207, a62+c208, a62+c209, a62+c210, a62+c211, a63+c1, a63+c2, a63+c3, a63+c4, a63+c5, a63+c6, a63+c7, a63+c8, a63+c9, a63+c10, a63+c11, a63+c12, a63+c13, a63+c14, a63+c15, a63+c16, a63+c17, a63+c18, a63+c19, a63+c20, a63+c21, a63+c22, a63+c23, a63+c24, a63+c25, a63+c26, a63+c27, a63+c28, a63+c29, a63+c30, a63+c31, a63+c32, a63+c33, a63+c34, a63+c35, a63+c36, a63+c37, a63+c38, a63+c39, a63+c40, a63+c41, a63+c42, a63+c43, a63+c44, a63+c45, a63+c46, a63+c47, a63+c48, a63+c49, a63+c50, a63+c51, a63+c52, a63+c53, a63+c54, a63+c55, a63+c56, a63+c57, a63+c58, a63+c59, a63+c60, a63+c61, a63+c62, a63+c63, a63+c64, a63+c65, a63+c66, a63+c67, a63+c68, a63+c69, a63+c70, a63+c71, a63+c72, a63+c73, a63+c74, a63+c75, a63+c76, a63+c77, a63+c78, a63+c79, a63+c80, a63+c81, a63+c82, a63+c83, a63+c84, a63+c85, a63+c86, a63+c87, a63+c88, a63+c89, a63+c90, a63+c91, a63+c92, a63+c93, a63+c94, a63+c95, a63+c96, a63+c97, a63+c98, a63+c99, a63+c100, a63+c101, a63+c102, a63+c103, a63+c104, a63+c105, a63+c106, a63+c107, a63+c108, a63+c109, a63+c110, a63+c111, a63+c112, a63+c113, a63+c114, a63+c115, a63+c116, a63+c117, a63+c118, a63+c119, a63+c120, a63+c121, a63+c122, a63+c123, a63+c124, a63+c125, a63+c126, a63+c127, a63+c128, a63+c129, a63+c130, a63+c131, a63+c132, a63+c133, a63+c134, a63+c135, a63+c136, a63+c137, a63+c138, a63+c139, a63+c140, a63+c141, a63+c142, a63+c143, a63+c144, a63+c145, a63+c146, a63+c147, a63+c148, a63+c149, a63+c150, a63+c151, a63+c152, a63+c153, a63+c154, a63+c155, a63+c156, a63+c157, a63+c158, a63+c159, a63+c160, a63+c161, a63+c162, a63+c163, a63+c164, a63+c165, a63+c166, a63+c167, a63+c168, a63+c169, a63+c170, a63+c171, a63+c172, a63+c173, a63+c174, a63+c175, a63+c176, a63+c177, a63+c178, a63+c179, a63+c180, a63+c181, a63+c182, a63+c183, a63+c184, a63+c185, a63+c186, a63+c187, a63+c188, a63+c189, a63+c190, a63+c191, a63+c192, a63+c193, a63+c194, a63+c195, a63+c196, a63+c197, a63+c198, a63+c199, a63+c200, a63+c201, a63+c202, a63+c203, a63+c204, a63+c205, a63+c206, a63+c207, a63+c208, a63+c209, a63+c210, a63+c211, a64+c1, a64+c2, a64+c3, a64+c4, a64+c5, a64+c6, a64+c7, a64+c8, a64+c9, a64+c10, a64+c11, a64+c12, a64+c13, a64+c14, a64+c15, a64+c16, a64+c17, a64+c18, a64+c19, a64+c20, a64+c21, a64+c22, a64+c23, a64+c24, a64+c25, a64+c26, a64+c27, a64+c28, a64+c29, a64+c30, a64+c31, a64+c32, a64+c33, a64+c34, a64+c35, a64+c36, a64+c37, a64+c38, a64+c39, a64+c40, a64+c41, a64+c42, a64+c43, a64+c44, a64+c45, a64+c46, a64+c47, a64+c48, a64+c49, a64+c50, a64+c51, a64+c52, a64+c53, a64+c54, a64+c55, a64+c56, a64+c57, a64+c58, a64+c59, a64+c60, a64+c61, a64+c62, a64+c63, a64+c64, a64+c65, a64+c66, a64+c67, a64+c68, a64+c69, a64+c70, a64+c71, a64+c72, a64+c73, a64+c74, a64+c75, a64+c76, a64+c77, a64+c78, a64+c79, a64+c80, a64+c81, a64+c82, a64+c83, a64+c84, a64+c85, a64+c86, a64+c87, a64+c88, a64+c89, a64+c90, a64+c91, a64+c92, a64+c93, a64+c94, a64+c95, a64+c96, a64+c97, a64+c98, a64+c99, a64+c100, a64+c101, a64+c102, a64+c103, a64+c104, a64+c105, a64+c106, a64+c107, a64+c108, a64+c109, a64+c110, a64+c111, a64+c112, a64+c113, a64+c114, a64+c115, a64+c116, a64+c117, a64+c118, a64+c119, a64+c120, a64+c121, a64+c122, a64+c123, a64+c124, a64+c125, a64+c126, a64+c127, a64+c128, a64+c129, a64+c130, a64+c131, a64+c132, a64+c133, a64+c134, a64+c135, a64+c136, a64+c137, a64+c138, a64+c139, a64+c140, a64+c141, a64+c142, a64+c143, a64+c144, a64+c145, a64+c146, a64+c147, a64+c148, a64+c149, a64+c150, a64+c151, a64+c152, a64+c153, a64+c154, a64+c155, a64+c156, a64+c157, a64+c158, a64+c159, a64+c160, a64+c161, a64+c162, a64+c163, a64+c164, a64+c165, a64+c166, a64+c167, a64+c168, a64+c169, a64+c170, a64+c171, a64+c172, a64+c173, a64+c174, a64+c175, a64+c176, a64+c177, a64+c178, a64+c179, a64+c180, a64+c181, a64+c182, a64+c183, a64+c184, a64+c185, a64+c186, a64+c187, a64+c188, a64+c189, a64+c190, a64+c191, a64+c192, a64+c193, a64+c194, a64+c195, a64+c196, a64+c197, a64+c198, a64+c199, a64+c200, a64+c201, a64+c202, a64+c203, a64+c204, a64+c205, a64+c206, a64+c207, a64+c208, a64+c209, a64+c210, a64+c211, a65+c1, a65+c2, a65+c3, a65+c4, a65+c5, a65+c6, a65+c7, a65+c8, a65+c9, a65+c10, a65+c11, a65+c12, a65+c13, a65+c14, a65+c15, a65+c16, a65+c17, a65+c18, a65+c19, a65+c20, a65+c21, a65+c22, a65+c23, a65+c24, a65+c25, a65+c26, a65+c27, a65+c28, a65+c29, a65+c30, a65+c31, a65+c32, a65+c33, a65+c34, a65+c35, a65+c36, a65+c37, a65+c38, a65+c39, a65+c40, a65+c41, a65+c42, a65+c43, a65+c44, a65+c45, a65+c46, a65+c47, a65+c48, a65+c49, a65+c50, a65+c51, a65+c52, a65+c53, a65+c54, a65+c55, a65+c56, a65+c57, a65+c58, a65+c59, a65+c60, a65+c61, a65+c62, a65+c63, a65+c64, a65+c65, a65+c66, a65+c67, a65+c68, a65+c69, a65+c70, a65+c71, a65+c72, a65+c73, a65+c74, a65+c75, a65+c76, a65+c77, a65+c78, a65+c79, a65+c80, a65+c81, a65+c82, a65+c83, a65+c84, a65+c85, a65+c86, a65+c87, a65+c88, a65+c89, a65+c90, a65+c91, a65+c92, a65+c93, a65+c94, a65+c95, a65+c96, a65+c97, a65+c98, a65+c99, a65+c100, a65+c101, a65+c102, a65+c103, a65+c104, a65+c105, a65+c106, a65+c107, a65+c108, a65+c109, a65+c110, a65+c111, a65+c112, a65+c113, a65+c114, a65+c115, a65+c116, a65+c117, a65+c118, a65+c119, a65+c120, a65+c121, a65+c122, a65+c123, a65+c124, a65+c125, a65+c126, a65+c127, a65+c128, a65+c129, a65+c130, a65+c131, a65+c132, a65+c133, a65+c134, a65+c135, a65+c136, a65+c137, a65+c138, a65+c139, a65+c140, a65+c141, a65+c142, a65+c143, a65+c144, a65+c145, a65+c146, a65+c147, a65+c148, a65+c149, a65+c150, a65+c151, a65+c152, a65+c153, a65+c154, a65+c155, a65+c156, a65+c157, a65+c158, a65+c159, a65+c160, a65+c161, a65+c162, a65+c163, a65+c164, a65+c165, a65+c166, a65+c167, a65+c168, a65+c169, a65+c170, a65+c171, a65+c172, a65+c173, a65+c174, a65+c175, a65+c176, a65+c177, a65+c178, a65+c179, a65+c180, a65+c181, a65+c182, a65+c183, a65+c184, a65+c185, a65+c186, a65+c187, a65+c188, a65+c189, a65+c190, a65+c191, a65+c192, a65+c193, a65+c194, a65+c195, a65+c196, a65+c197, a65+c198, a65+c199, a65+c200, a65+c201, a65+c202, a65+c203, a65+c204, a65+c205, a65+c206, a65+c207, a65+c208, a65+c209, a65+c210, a65+c211, a66+c1, a66+c2, a66+c3, a66+c4, a66+c5, a66+c6, a66+c7, a66+c8, a66+c9, a66+c10, a66+c11, a66+c12, a66+c13, a66+c14, a66+c15, a66+c16, a66+c17, a66+c18, a66+c19, a66+c20, a66+c21, a66+c22, a66+c23, a66+c24, a66+c25, a66+c26, a66+c27, a66+c28, a66+c29, a66+c30, a66+c31, a66+c32, a66+c33, a66+c34, a66+c35, a66+c36, a66+c37, a66+c38, a66+c39, a66+c40, a66+c41, a66+c42, a66+c43, a66+c44, a66+c45, a66+c46, a66+c47, a66+c48, a66+c49, a66+c50, a66+c51, a66+c52, a66+c53, a66+c54, a66+c55, a66+c56, a66+c57, a66+c58, a66+c59, a66+c60, a66+c61, a66+c62, a66+c63, a66+c64, a66+c65, a66+c66, a66+c67, a66+c68, a66+c69, a66+c70, a66+c71, a66+c72, a66+c73, a66+c74, a66+c75, a66+c76, a66+c77, a66+c78, a66+c79, a66+c80, a66+c81, a66+c82, a66+c83, a66+c84, a66+c85, a66+c86, a66+c87, a66+c88, a66+c89, a66+c90, a66+c91, a66+c92, a66+c93, a66+c94, a66+c95, a66+c96, a66+c97, a66+c98, a66+c99, a66+c100, a66+c101, a66+c102, a66+c103, a66+c104, a66+c105, a66+c106, a66+c107, a66+c108, a66+c109, a66+c110, a66+c111, a66+c112, a66+c113, a66+c114, a66+c115, a66+c116, a66+c117, a66+c118, a66+c119, a66+c120, a66+c121, a66+c122, a66+c123, a66+c124, a66+c125, a66+c126, a66+c127, a66+c128, a66+c129, a66+c130, a66+c131, a66+c132, a66+c133, a66+c134, a66+c135, a66+c136, a66+c137, a66+c138, a66+c139, a66+c140, a66+c141, a66+c142, a66+c143, a66+c144, a66+c145, a66+c146, a66+c147, a66+c148, a66+c149, a66+c150, a66+c151, a66+c152, a66+c153, a66+c154, a66+c155, a66+c156, a66+c157, a66+c158, a66+c159, a66+c160, a66+c161, a66+c162, a66+c163, a66+c164, a66+c165, a66+c166, a66+c167, a66+c168, a66+c169, a66+c170, a66+c171, a66+c172, a66+c173, a66+c174, a66+c175, a66+c176, a66+c177, a66+c178, a66+c179, a66+c180, a66+c181, a66+c182, a66+c183, a66+c184, a66+c185, a66+c186, a66+c187, a66+c188, a66+c189, a66+c190, a66+c191, a66+c192, a66+c193, a66+c194, a66+c195, a66+c196, a66+c197, a66+c198, a66+c199, a66+c200, a66+c201, a66+c202, a66+c203, a66+c204, a66+c205, a66+c206, a66+c207, a66+c208, a66+c209, a66+c210, a66+c211, a67+c1, a67+c2, a67+c3, a67+c4, a67+c5, a67+c6, a67+c7, a67+c8, a67+c9, a67+c10, a67+c11, a67+c12, a67+c13, a67+c14, a67+c15, a67+c16, a67+c17, a67+c18, a67+c19, a67+c20, a67+c21, a67+c22, a67+c23, a67+c24, a67+c25, a67+c26, a67+c27, a67+c28, a67+c29, a67+c30, a67+c31, a67+c32, a67+c33, a67+c34, a67+c35, a67+c36, a67+c37, a67+c38, a67+c39, a67+c40, a67+c41, a67+c42, a67+c43, a67+c44, a67+c45, a67+c46, a67+c47, a67+c48, a67+c49, a67+c50, a67+c51, a67+c52, a67+c53, a67+c54, a67+c55, a67+c56, a67+c57, a67+c58, a67+c59, a67+c60, a67+c61, a67+c62, a67+c63, a67+c64, a67+c65, a67+c66, a67+c67, a67+c68, a67+c69, a67+c70, a67+c71, a67+c72, a67+c73, a67+c74, a67+c75, a67+c76, a67+c77, a67+c78, a67+c79, a67+c80, a67+c81, a67+c82, a67+c83, a67+c84, a67+c85, a67+c86, a67+c87, a67+c88, a67+c89, a67+c90, a67+c91, a67+c92, a67+c93, a67+c94, a67+c95, a67+c96, a67+c97, a67+c98, a67+c99, a67+c100, a67+c101, a67+c102, a67+c103, a67+c104, a67+c105, a67+c106, a67+c107, a67+c108, a67+c109, a67+c110, a67+c111, a67+c112, a67+c113, a67+c114, a67+c115, a67+c116, a67+c117, a67+c118, a67+c119, a67+c120, a67+c121, a67+c122, a67+c123, a67+c124, a67+c125, a67+c126, a67+c127, a67+c128, a67+c129, a67+c130, a67+c131, a67+c132, a67+c133, a67+c134, a67+c135, a67+c136, a67+c137, a67+c138, a67+c139, a67+c140, a67+c141, a67+c142, a67+c143, a67+c144, a67+c145, a67+c146, a67+c147, a67+c148, a67+c149, a67+c150, a67+c151, a67+c152, a67+c153, a67+c154, a67+c155, a67+c156, a67+c157, a67+c158, a67+c159, a67+c160, a67+c161, a67+c162, a67+c163, a67+c164, a67+c165, a67+c166, a67+c167, a67+c168, a67+c169, a67+c170, a67+c171, a67+c172, a67+c173, a67+c174, a67+c175, a67+c176, a67+c177, a67+c178, a67+c179, a67+c180, a67+c181, a67+c182, a67+c183, a67+c184, a67+c185, a67+c186, a67+c187, a67+c188, a67+c189, a67+c190, a67+c191, a67+c192, a67+c193, a67+c194, a67+c195, a67+c196, a67+c197, a67+c198, a67+c199, a67+c200, a67+c201, a67+c202, a67+c203, a67+c204, a67+c205, a67+c206, a67+c207, a67+c208, a67+c209, a67+c210, a67+c211, a68+c1, a68+c2, a68+c3, a68+c4, a68+c5, a68+c6, a68+c7, a68+c8, a68+c9, a68+c10, a68+c11, a68+c12, a68+c13, a68+c14, a68+c15, a68+c16, a68+c17, a68+c18, a68+c19, a68+c20, a68+c21, a68+c22, a68+c23, a68+c24, a68+c25, a68+c26, a68+c27, a68+c28, a68+c29, a68+c30, a68+c31, a68+c32, a68+c33, a68+c34, a68+c35, a68+c36, a68+c37, a68+c38, a68+c39, a68+c40, a68+c41, a68+c42, a68+c43, a68+c44, a68+c45, a68+c46, a68+c47, a68+c48, a68+c49, a68+c50, a68+c51, a68+c52, a68+c53, a68+c54, a68+c55, a68+c56, a68+c57, a68+c58, a68+c59, a68+c60, a68+c61, a68+c62, a68+c63, a68+c64, a68+c65, a68+c66, a68+c67, a68+c68, a68+c69, a68+c70, a68+c71, a68+c72, a68+c73, a68+c74, a68+c75, a68+c76, a68+c77, a68+c78, a68+c79, a68+c80, a68+c81, a68+c82, a68+c83, a68+c84, a68+c85, a68+c86, a68+c87, a68+c88, a68+c89, a68+c90, a68+c91, a68+c92, a68+c93, a68+c94, a68+c95, a68+c96, a68+c97, a68+c98, a68+c99, a68+c100, a68+c101, a68+c102, a68+c103, a68+c104, a68+c105, a68+c106, a68+c107, a68+c108, a68+c109, a68+c110, a68+c111, a68+c112, a68+c113, a68+c114, a68+c115, a68+c116, a68+c117, a68+c118, a68+c119, a68+c120, a68+c121, a68+c122, a68+c123, a68+c124, a68+c125, a68+c126, a68+c127, a68+c128, a68+c129, a68+c130, a68+c131, a68+c132, a68+c133, a68+c134, a68+c135, a68+c136, a68+c137, a68+c138, a68+c139, a68+c140, a68+c141, a68+c142, a68+c143, a68+c144, a68+c145, a68+c146, a68+c147, a68+c148, a68+c149, a68+c150, a68+c151, a68+c152, a68+c153, a68+c154, a68+c155, a68+c156, a68+c157, a68+c158, a68+c159, a68+c160, a68+c161, a68+c162, a68+c163, a68+c164, a68+c165, a68+c166, a68+c167, a68+c168, a68+c169, a68+c170, a68+c171, a68+c172, a68+c173, a68+c174, a68+c175, a68+c176, a68+c177, a68+c178, a68+c179, a68+c180, a68+c181, a68+c182, a68+c183, a68+c184, a68+c185, a68+c186, a68+c187, a68+c188, a68+c189, a68+c190, a68+c191, a68+c192, a68+c193, a68+c194, a68+c195, a68+c196, a68+c197, a68+c198, a68+c199, a68+c200, a68+c201, a68+c202, a68+c203, a68+c204, a68+c205, a68+c206, a68+c207, a68+c208, a68+c209, a68+c210, a68+c211, a69+c1, a69+c2, a69+c3, a69+c4, a69+c5, a69+c6, a69+c7, a69+c8, a69+c9, a69+c10, a69+c11, a69+c12, a69+c13, a69+c14, a69+c15, a69+c16, a69+c17, a69+c18, a69+c19, a69+c20, a69+c21, a69+c22, a69+c23, a69+c24, a69+c25, a69+c26, a69+c27, a69+c28, a69+c29, a69+c30, a69+c31, a69+c32, a69+c33, a69+c34, a69+c35, a69+c36, a69+c37, a69+c38, a69+c39, a69+c40, a69+c41, a69+c42, a69+c43, a69+c44, a69+c45, a69+c46, a69+c47, a69+c48, a69+c49, a69+c50, a69+c51, a69+c52, a69+c53, a69+c54, a69+c55, a69+c56, a69+c57, a69+c58, a69+c59, a69+c60, a69+c61, a69+c62, a69+c63, a69+c64, a69+c65, a69+c66, a69+c67, a69+c68, a69+c69, a69+c70, a69+c71, a69+c72, a69+c73, a69+c74, a69+c75, a69+c76, a69+c77, a69+c78, a69+c79, a69+c80, a69+c81, a69+c82, a69+c83, a69+c84, a69+c85, a69+c86, a69+c87, a69+c88, a69+c89, a69+c90, a69+c91, a69+c92, a69+c93, a69+c94, a69+c95, a69+c96, a69+c97, a69+c98, a69+c99, a69+c100, a69+c101, a69+c102, a69+c103, a69+c104, a69+c105, a69+c106, a69+c107, a69+c108, a69+c109, a69+c110, a69+c111, a69+c112, a69+c113, a69+c114, a69+c115, a69+c116, a69+c117, a69+c118, a69+c119, a69+c120, a69+c121, a69+c122, a69+c123, a69+c124, a69+c125, a69+c126, a69+c127, a69+c128, a69+c129, a69+c130, a69+c131, a69+c132, a69+c133, a69+c134, a69+c135, a69+c136, a69+c137, a69+c138, a69+c139, a69+c140, a69+c141, a69+c142, a69+c143, a69+c144, a69+c145, a69+c146, a69+c147, a69+c148, a69+c149, a69+c150, a69+c151, a69+c152, a69+c153, a69+c154, a69+c155, a69+c156, a69+c157, a69+c158, a69+c159, a69+c160, a69+c161, a69+c162, a69+c163, a69+c164, a69+c165, a69+c166, a69+c167, a69+c168, a69+c169, a69+c170, a69+c171, a69+c172, a69+c173, a69+c174, a69+c175, a69+c176, a69+c177, a69+c178, a69+c179, a69+c180, a69+c181, a69+c182, a69+c183, a69+c184, a69+c185, a69+c186, a69+c187, a69+c188, a69+c189, a69+c190, a69+c191, a69+c192, a69+c193, a69+c194, a69+c195, a69+c196, a69+c197, a69+c198, a69+c199, a69+c200, a69+c201, a69+c202, a69+c203, a69+c204, a69+c205, a69+c206, a69+c207, a69+c208, a69+c209, a69+c210, a69+c211, a70+c1, a70+c2, a70+c3, a70+c4, a70+c5, a70+c6, a70+c7, a70+c8, a70+c9, a70+c10, a70+c11, a70+c12, a70+c13, a70+c14, a70+c15, a70+c16, a70+c17, a70+c18, a70+c19, a70+c20, a70+c21, a70+c22, a70+c23, a70+c24, a70+c25, a70+c26, a70+c27, a70+c28, a70+c29, a70+c30, a70+c31, a70+c32, a70+c33, a70+c34, a70+c35, a70+c36, a70+c37, a70+c38, a70+c39, a70+c40, a70+c41, a70+c42, a70+c43, a70+c44, a70+c45, a70+c46, a70+c47, a70+c48, a70+c49, a70+c50, a70+c51, a70+c52, a70+c53, a70+c54, a70+c55, a70+c56, a70+c57, a70+c58, a70+c59, a70+c60, a70+c61, a70+c62, a70+c63, a70+c64, a70+c65, a70+c66, a70+c67, a70+c68, a70+c69, a70+c70, a70+c71, a70+c72, a70+c73, a70+c74, a70+c75, a70+c76, a70+c77, a70+c78, a70+c79, a70+c80, a70+c81, a70+c82, a70+c83, a70+c84, a70+c85, a70+c86, a70+c87, a70+c88, a70+c89, a70+c90, a70+c91, a70+c92, a70+c93, a70+c94, a70+c95, a70+c96, a70+c97, a70+c98, a70+c99, a70+c100, a70+c101, a70+c102, a70+c103, a70+c104, a70+c105, a70+c106, a70+c107, a70+c108, a70+c109, a70+c110, a70+c111, a70+c112, a70+c113, a70+c114, a70+c115, a70+c116, a70+c117, a70+c118, a70+c119, a70+c120, a70+c121, a70+c122, a70+c123, a70+c124, a70+c125, a70+c126, a70+c127, a70+c128, a70+c129, a70+c130, a70+c131, a70+c132, a70+c133, a70+c134, a70+c135, a70+c136, a70+c137, a70+c138, a70+c139, a70+c140, a70+c141, a70+c142, a70+c143, a70+c144, a70+c145, a70+c146, a70+c147, a70+c148, a70+c149, a70+c150, a70+c151, a70+c152, a70+c153, a70+c154, a70+c155, a70+c156, a70+c157, a70+c158, a70+c159, a70+c160, a70+c161, a70+c162, a70+c163, a70+c164, a70+c165, a70+c166, a70+c167, a70+c168, a70+c169, a70+c170, a70+c171, a70+c172, a70+c173, a70+c174, a70+c175, a70+c176, a70+c177, a70+c178, a70+c179, a70+c180, a70+c181, a70+c182, a70+c183, a70+c184, a70+c185, a70+c186, a70+c187, a70+c188, a70+c189, a70+c190, a70+c191, a70+c192, a70+c193, a70+c194, a70+c195, a70+c196, a70+c197, a70+c198, a70+c199, a70+c200, a70+c201, a70+c202, a70+c203, a70+c204, a70+c205, a70+c206, a70+c207, a70+c208, a70+c209, a70+c210, a70+c211, a71+c1, a71+c2, a71+c3, a71+c4, a71+c5, a71+c6, a71+c7, a71+c8, a71+c9, a71+c10, a71+c11, a71+c12, a71+c13, a71+c14, a71+c15, a71+c16, a71+c17, a71+c18, a71+c19, a71+c20, a71+c21, a71+c22, a71+c23, a71+c24, a71+c25, a71+c26, a71+c27, a71+c28, a71+c29, a71+c30, a71+c31, a71+c32, a71+c33, a71+c34, a71+c35, a71+c36, a71+c37, a71+c38, a71+c39, a71+c40, a71+c41, a71+c42, a71+c43, a71+c44, a71+c45, a71+c46, a71+c47, a71+c48, a71+c49, a71+c50, a71+c51, a71+c52, a71+c53, a71+c54, a71+c55, a71+c56, a71+c57, a71+c58, a71+c59, a71+c60, a71+c61, a71+c62, a71+c63, a71+c64, a71+c65, a71+c66, a71+c67, a71+c68, a71+c69, a71+c70, a71+c71, a71+c72, a71+c73, a71+c74, a71+c75, a71+c76, a71+c77, a71+c78, a71+c79, a71+c80, a71+c81, a71+c82, a71+c83, a71+c84, a71+c85, a71+c86, a71+c87, a71+c88, a71+c89, a71+c90, a71+c91, a71+c92, a71+c93, a71+c94, a71+c95, a71+c96, a71+c97, a71+c98, a71+c99, a71+c100, a71+c101, a71+c102, a71+c103, a71+c104, a71+c105, a71+c106, a71+c107, a71+c108, a71+c109, a71+c110, a71+c111, a71+c112, a71+c113, a71+c114, a71+c115, a71+c116, a71+c117, a71+c118, a71+c119, a71+c120, a71+c121, a71+c122, a71+c123, a71+c124, a71+c125, a71+c126, a71+c127, a71+c128, a71+c129, a71+c130, a71+c131, a71+c132, a71+c133, a71+c134, a71+c135, a71+c136, a71+c137, a71+c138, a71+c139, a71+c140, a71+c141, a71+c142, a71+c143, a71+c144, a71+c145, a71+c146, a71+c147, a71+c148, a71+c149, a71+c150, a71+c151, a71+c152, a71+c153, a71+c154, a71+c155, a71+c156, a71+c157, a71+c158, a71+c159, a71+c160, a71+c161, a71+c162, a71+c163, a71+c164, a71+c165, a71+c166, a71+c167, a71+c168, a71+c169, a71+c170, a71+c171, a71+c172, a71+c173, a71+c174, a71+c175, a71+c176, a71+c177, a71+c178, a71+c179, a71+c180, a71+c181, a71+c182, a71+c183, a71+c184, a71+c185, a71+c186, a71+c187, a71+c188, a71+c189, a71+c190, a71+c191, a71+c192, a71+c193, a71+c194, a71+c195, a71+c196, a71+c197, a71+c198, a71+c199, a71+c200, a71+c201, a71+c202, a71+c203, a71+c204, a71+c205, a71+c206, a71+c207, a71+c208, a71+c209, a71+c210, a71+c211, a72+c1, a72+c2, a72+c3, a72+c4, a72+c5, a72+c6, a72+c7, a72+c8, a72+c9, a72+c10, a72+c11, a72+c12, a72+c13, a72+c14, a72+c15, a72+c16, a72+c17, a72+c18, a72+c19, a72+c20, a72+c21, a72+c22, a72+c23, a72+c24, a72+c25, a72+c26, a72+c27, a72+c28, a72+c29, a72+c30, a72+c31, a72+c32, a72+c33, a72+c34, a72+c35, a72+c36, a72+c37, a72+c38, a72+c39, a72+c40, a72+c41, a72+c42, a72+c43, a72+c44, a72+c45, a72+c46, a72+c47, a72+c48, a72+c49, a72+c50, a72+c51, a72+c52, a72+c53, a72+c54, a72+c55, a72+c56, a72+c57, a72+c58, a72+c59, a72+c60, a72+c61, a72+c62, a72+c63, a72+c64, a72+c65, a72+c66, a72+c67, a72+c68, a72+c69, a72+c70, a72+c71, a72+c72, a72+c73, a72+c74, a72+c75, a72+c76, a72+c77, a72+c78, a72+c79, a72+c80, a72+c81, a72+c82, a72+c83, a72+c84, a72+c85, a72+c86, a72+c87, a72+c88, a72+c89, a72+c90, a72+c91, a72+c92, a72+c93, a72+c94, a72+c95, a72+c96, a72+c97, a72+c98, a72+c99, a72+c100, a72+c101, a72+c102, a72+c103, a72+c104, a72+c105, a72+c106, a72+c107, a72+c108, a72+c109, a72+c110, a72+c111, a72+c112, a72+c113, a72+c114, a72+c115, a72+c116, a72+c117, a72+c118, a72+c119, a72+c120, a72+c121, a72+c122, a72+c123, a72+c124, a72+c125, a72+c126, a72+c127, a72+c128, a72+c129, a72+c130, a72+c131, a72+c132, a72+c133, a72+c134, a72+c135, a72+c136, a72+c137, a72+c138, a72+c139, a72+c140, a72+c141, a72+c142, a72+c143, a72+c144, a72+c145, a72+c146, a72+c147, a72+c148, a72+c149, a72+c150, a72+c151, a72+c152, a72+c153, a72+c154, a72+c155, a72+c156, a72+c157, a72+c158, a72+c159, a72+c160, a72+c161, a72+c162, a72+c163, a72+c164, a72+c165, a72+c166, a72+c167, a72+c168, a72+c169, a72+c170, a72+c171, a72+c172, a72+c173, a72+c174, a72+c175, a72+c176, a72+c177, a72+c178, a72+c179, a72+c180, a72+c181, a72+c182, a72+c183, a72+c184, a72+c185, a72+c186, a72+c187, a72+c188, a72+c189, a72+c190, a72+c191, a72+c192, a72+c193, a72+c194, a72+c195, a72+c196, a72+c197, a72+c198, a72+c199, a72+c200, a72+c201, a72+c202, a72+c203, a72+c204, a72+c205, a72+c206, a72+c207, a72+c208, a72+c209, a72+c210, a72+c211, a73+c1, a73+c2, a73+c3, a73+c4, a73+c5, a73+c6, a73+c7, a73+c8, a73+c9, a73+c10, a73+c11, a73+c12, a73+c13, a73+c14, a73+c15, a73+c16, a73+c17, a73+c18, a73+c19, a73+c20, a73+c21, a73+c22, a73+c23, a73+c24, a73+c25, a73+c26, a73+c27, a73+c28, a73+c29, a73+c30, a73+c31, a73+c32, a73+c33, a73+c34, a73+c35, a73+c36, a73+c37, a73+c38, a73+c39, a73+c40, a73+c41, a73+c42, a73+c43, a73+c44, a73+c45, a73+c46, a73+c47, a73+c48, a73+c49, a73+c50, a73+c51, a73+c52, a73+c53, a73+c54, a73+c55, a73+c56, a73+c57, a73+c58, a73+c59, a73+c60, a73+c61, a73+c62, a73+c63, a73+c64, a73+c65, a73+c66, a73+c67, a73+c68, a73+c69, a73+c70, a73+c71, a73+c72, a73+c73, a73+c74, a73+c75, a73+c76, a73+c77, a73+c78, a73+c79, a73+c80, a73+c81, a73+c82, a73+c83, a73+c84, a73+c85, a73+c86, a73+c87, a73+c88, a73+c89, a73+c90, a73+c91, a73+c92, a73+c93, a73+c94, a73+c95, a73+c96, a73+c97, a73+c98, a73+c99, a73+c100, a73+c101, a73+c102, a73+c103, a73+c104, a73+c105, a73+c106, a73+c107, a73+c108, a73+c109, a73+c110, a73+c111, a73+c112, a73+c113, a73+c114, a73+c115, a73+c116, a73+c117, a73+c118, a73+c119, a73+c120, a73+c121, a73+c122, a73+c123, a73+c124, a73+c125, a73+c126, a73+c127, a73+c128, a73+c129, a73+c130, a73+c131, a73+c132, a73+c133, a73+c134, a73+c135, a73+c136, a73+c137, a73+c138, a73+c139, a73+c140, a73+c141, a73+c142, a73+c143, a73+c144, a73+c145, a73+c146, a73+c147, a73+c148, a73+c149, a73+c150, a73+c151, a73+c152, a73+c153, a73+c154, a73+c155, a73+c156, a73+c157, a73+c158, a73+c159, a73+c160, a73+c161, a73+c162, a73+c163, a73+c164, a73+c165, a73+c166, a73+c167, a73+c168, a73+c169, a73+c170, a73+c171, a73+c172, a73+c173, a73+c174, a73+c175, a73+c176, a73+c177, a73+c178, a73+c179, a73+c180, a73+c181, a73+c182, a73+c183, a73+c184, a73+c185, a73+c186, a73+c187, a73+c188, a73+c189, a73+c190, a73+c191, a73+c192, a73+c193, a73+c194, a73+c195, a73+c196, a73+c197, a73+c198, a73+c199, a73+c200, a73+c201, a73+c202, a73+c203, a73+c204, a73+c205, a73+c206, a73+c207, a73+c208, a73+c209, a73+c210, a73+c211, a74+c1, a74+c2, a74+c3, a74+c4, a74+c5, a74+c6, a74+c7, a74+c8, a74+c9, a74+c10, a74+c11, a74+c12, a74+c13, a74+c14, a74+c15, a74+c16, a74+c17, a74+c18, a74+c19, a74+c20, a74+c21, a74+c22, a74+c23, a74+c24, a74+c25, a74+c26, a74+c27, a74+c28, a74+c29, a74+c30, a74+c31, a74+c32, a74+c33, a74+c34, a74+c35, a74+c36, a74+c37, a74+c38, a74+c39, a74+c40, a74+c41, a74+c42, a74+c43, a74+c44, a74+c45, a74+c46, a74+c47, a74+c48, a74+c49, a74+c50, a74+c51, a74+c52, a74+c53, a74+c54, a74+c55, a74+c56, a74+c57, a74+c58, a74+c59, a74+c60, a74+c61, a74+c62, a74+c63, a74+c64, a74+c65, a74+c66, a74+c67, a74+c68, a74+c69, a74+c70, a74+c71, a74+c72, a74+c73, a74+c74, a74+c75, a74+c76, a74+c77, a74+c78, a74+c79, a74+c80, a74+c81, a74+c82, a74+c83, a74+c84, a74+c85, a74+c86, a74+c87, a74+c88, a74+c89, a74+c90, a74+c91, a74+c92, a74+c93, a74+c94, a74+c95, a74+c96, a74+c97, a74+c98, a74+c99, a74+c100, a74+c101, a74+c102, a74+c103, a74+c104, a74+c105, a74+c106, a74+c107, a74+c108, a74+c109, a74+c110, a74+c111, a74+c112, a74+c113, a74+c114, a74+c115, a74+c116, a74+c117, a74+c118, a74+c119, a74+c120, a74+c121, a74+c122, a74+c123, a74+c124, a74+c125, a74+c126, a74+c127, a74+c128, a74+c129, a74+c130, a74+c131, a74+c132, a74+c133, a74+c134, a74+c135, a74+c136, a74+c137, a74+c138, a74+c139, a74+c140, a74+c141, a74+c142, a74+c143, a74+c144, a74+c145, a74+c146, a74+c147, a74+c148, a74+c149, a74+c150, a74+c151, a74+c152, a74+c153, a74+c154, a74+c155, a74+c156, a74+c157, a74+c158, a74+c159, a74+c160, a74+c161, a74+c162, a74+c163, a74+c164, a74+c165, a74+c166, a74+c167, a74+c168, a74+c169, a74+c170, a74+c171, a74+c172, a74+c173, a74+c174, a74+c175, a74+c176, a74+c177, a74+c178, a74+c179, a74+c180, a74+c181, a74+c182, a74+c183, a74+c184, a74+c185, a74+c186, a74+c187, a74+c188, a74+c189, a74+c190, a74+c191, a74+c192, a74+c193, a74+c194, a74+c195, a74+c196, a74+c197, a74+c198, a74+c199, a74+c200, a74+c201, a74+c202, a74+c203, a74+c204, a74+c205, a74+c206, a74+c207, a74+c208, a74+c209, a74+c210, a74+c211, a75+c1, a75+c2, a75+c3, a75+c4, a75+c5, a75+c6, a75+c7, a75+c8, a75+c9, a75+c10, a75+c11, a75+c12, a75+c13, a75+c14, a75+c15, a75+c16, a75+c17, a75+c18, a75+c19, a75+c20, a75+c21, a75+c22, a75+c23, a75+c24, a75+c25, a75+c26, a75+c27, a75+c28, a75+c29, a75+c30, a75+c31, a75+c32, a75+c33, a75+c34, a75+c35, a75+c36, a75+c37, a75+c38, a75+c39, a75+c40, a75+c41, a75+c42, a75+c43, a75+c44, a75+c45, a75+c46, a75+c47, a75+c48, a75+c49, a75+c50, a75+c51, a75+c52, a75+c53, a75+c54, a75+c55, a75+c56, a75+c57, a75+c58, a75+c59, a75+c60, a75+c61, a75+c62, a75+c63, a75+c64, a75+c65, a75+c66, a75+c67, a75+c68, a75+c69, a75+c70, a75+c71, a75+c72, a75+c73, a75+c74, a75+c75, a75+c76, a75+c77, a75+c78, a75+c79, a75+c80, a75+c81, a75+c82, a75+c83, a75+c84, a75+c85, a75+c86, a75+c87, a75+c88, a75+c89, a75+c90, a75+c91, a75+c92, a75+c93, a75+c94, a75+c95, a75+c96, a75+c97, a75+c98, a75+c99, a75+c100, a75+c101, a75+c102, a75+c103, a75+c104, a75+c105, a75+c106, a75+c107, a75+c108, a75+c109, a75+c110, a75+c111, a75+c112, a75+c113, a75+c114, a75+c115, a75+c116, a75+c117, a75+c118, a75+c119, a75+c120, a75+c121, a75+c122, a75+c123, a75+c124, a75+c125, a75+c126, a75+c127, a75+c128, a75+c129, a75+c130, a75+c131, a75+c132, a75+c133, a75+c134, a75+c135, a75+c136, a75+c137, a75+c138, a75+c139, a75+c140, a75+c141, a75+c142, a75+c143, a75+c144, a75+c145, a75+c146, a75+c147, a75+c148, a75+c149, a75+c150, a75+c151, a75+c152, a75+c153, a75+c154, a75+c155, a75+c156, a75+c157, a75+c158, a75+c159, a75+c160, a75+c161, a75+c162, a75+c163, a75+c164, a75+c165, a75+c166, a75+c167, a75+c168, a75+c169, a75+c170, a75+c171, a75+c172, a75+c173, a75+c174, a75+c175, a75+c176, a75+c177, a75+c178, a75+c179, a75+c180, a75+c181, a75+c182, a75+c183, a75+c184, a75+c185, a75+c186, a75+c187, a75+c188, a75+c189, a75+c190, a75+c191, a75+c192, a75+c193, a75+c194, a75+c195, a75+c196, a75+c197, a75+c198, a75+c199, a75+c200, a75+c201, a75+c202, a75+c203, a75+c204, a75+c205, a75+c206, a75+c207, a75+c208, a75+c209, a75+c210, a75+c211, a76+c1, a76+c2, a76+c3, a76+c4, a76+c5, a76+c6, a76+c7, a76+c8, a76+c9, a76+c10, a76+c11, a76+c12, a76+c13, a76+c14, a76+c15, a76+c16, a76+c17, a76+c18, a76+c19, a76+c20, a76+c21, a76+c22, a76+c23, a76+c24, a76+c25, a76+c26, a76+c27, a76+c28, a76+c29, a76+c30, a76+c31, a76+c32, a76+c33, a76+c34, a76+c35, a76+c36, a76+c37, a76+c38, a76+c39, a76+c40, a76+c41, a76+c42, a76+c43, a76+c44, a76+c45, a76+c46, a76+c47, a76+c48, a76+c49, a76+c50, a76+c51, a76+c52, a76+c53, a76+c54, a76+c55, a76+c56, a76+c57, a76+c58, a76+c59, a76+c60, a76+c61, a76+c62, a76+c63, a76+c64, a76+c65, a76+c66, a76+c67, a76+c68, a76+c69, a76+c70, a76+c71, a76+c72, a76+c73, a76+c74, a76+c75, a76+c76, a76+c77, a76+c78, a76+c79, a76+c80, a76+c81, a76+c82, a76+c83, a76+c84, a76+c85, a76+c86, a76+c87, a76+c88, a76+c89, a76+c90, a76+c91, a76+c92, a76+c93, a76+c94, a76+c95, a76+c96, a76+c97, a76+c98, a76+c99, a76+c100, a76+c101, a76+c102, a76+c103, a76+c104, a76+c105, a76+c106, a76+c107, a76+c108, a76+c109, a76+c110, a76+c111, a76+c112, a76+c113, a76+c114, a76+c115, a76+c116, a76+c117, a76+c118, a76+c119, a76+c120, a76+c121, a76+c122, a76+c123, a76+c124, a76+c125, a76+c126, a76+c127, a76+c128, a76+c129, a76+c130, a76+c131, a76+c132, a76+c133, a76+c134, a76+c135, a76+c136, a76+c137, a76+c138, a76+c139, a76+c140, a76+c141, a76+c142, a76+c143, a76+c144, a76+c145, a76+c146, a76+c147, a76+c148, a76+c149, a76+c150, a76+c151, a76+c152, a76+c153, a76+c154, a76+c155, a76+c156, a76+c157, a76+c158, a76+c159, a76+c160, a76+c161, a76+c162, a76+c163, a76+c164, a76+c165, a76+c166, a76+c167, a76+c168, a76+c169, a76+c170, a76+c171, a76+c172, a76+c173, a76+c174, a76+c175, a76+c176, a76+c177, a76+c178, a76+c179, a76+c180, a76+c181, a76+c182, a76+c183, a76+c184, a76+c185, a76+c186, a76+c187, a76+c188, a76+c189, a76+c190, a76+c191, a76+c192, a76+c193, a76+c194, a76+c195, a76+c196, a76+c197, a76+c198, a76+c199, a76+c200, a76+c201, a76+c202, a76+c203, a76+c204, a76+c205, a76+c206, a76+c207, a76+c208, a76+c209, a76+c210, a76+c211, a77+c1, a77+c2, a77+c3, a77+c4, a77+c5, a77+c6, a77+c7, a77+c8, a77+c9, a77+c10, a77+c11, a77+c12, a77+c13, a77+c14, a77+c15, a77+c16, a77+c17, a77+c18, a77+c19, a77+c20, a77+c21, a77+c22, a77+c23, a77+c24, a77+c25, a77+c26, a77+c27, a77+c28, a77+c29, a77+c30, a77+c31, a77+c32, a77+c33, a77+c34, a77+c35, a77+c36, a77+c37, a77+c38, a77+c39, a77+c40, a77+c41, a77+c42, a77+c43, a77+c44, a77+c45, a77+c46, a77+c47, a77+c48, a77+c49, a77+c50, a77+c51, a77+c52, a77+c53, a77+c54, a77+c55, a77+c56, a77+c57, a77+c58, a77+c59, a77+c60, a77+c61, a77+c62, a77+c63, a77+c64, a77+c65, a77+c66, a77+c67, a77+c68, a77+c69, a77+c70, a77+c71, a77+c72, a77+c73, a77+c74, a77+c75, a77+c76, a77+c77, a77+c78, a77+c79, a77+c80, a77+c81, a77+c82, a77+c83, a77+c84, a77+c85, a77+c86, a77+c87, a77+c88, a77+c89, a77+c90, a77+c91, a77+c92, a77+c93, a77+c94, a77+c95, a77+c96, a77+c97, a77+c98, a77+c99, a77+c100, a77+c101, a77+c102, a77+c103, a77+c104, a77+c105, a77+c106, a77+c107, a77+c108, a77+c109, a77+c110, a77+c111, a77+c112, a77+c113, a77+c114, a77+c115, a77+c116, a77+c117, a77+c118, a77+c119, a77+c120, a77+c121, a77+c122, a77+c123, a77+c124, a77+c125, a77+c126, a77+c127, a77+c128, a77+c129, a77+c130, a77+c131, a77+c132, a77+c133, a77+c134, a77+c135, a77+c136, a77+c137, a77+c138, a77+c139, a77+c140, a77+c141, a77+c142, a77+c143, a77+c144, a77+c145, a77+c146, a77+c147, a77+c148, a77+c149, a77+c150, a77+c151, a77+c152, a77+c153, a77+c154, a77+c155, a77+c156, a77+c157, a77+c158, a77+c159, a77+c160, a77+c161, a77+c162, a77+c163, a77+c164, a77+c165, a77+c166, a77+c167, a77+c168, a77+c169, a77+c170, a77+c171, a77+c172, a77+c173, a77+c174, a77+c175, a77+c176, a77+c177, a77+c178, a77+c179, a77+c180, a77+c181, a77+c182, a77+c183, a77+c184, a77+c185, a77+c186, a77+c187, a77+c188, a77+c189, a77+c190, a77+c191, a77+c192, a77+c193, a77+c194, a77+c195, a77+c196, a77+c197, a77+c198, a77+c199, a77+c200, a77+c201, a77+c202, a77+c203, a77+c204, a77+c205, a77+c206, a77+c207, a77+c208, a77+c209, a77+c210, a77+c211, a78+c1, a78+c2, a78+c3, a78+c4, a78+c5, a78+c6, a78+c7, a78+c8, a78+c9, a78+c10, a78+c11, a78+c12, a78+c13, a78+c14, a78+c15, a78+c16, a78+c17, a78+c18, a78+c19, a78+c20, a78+c21, a78+c22, a78+c23, a78+c24, a78+c25, a78+c26, a78+c27, a78+c28, a78+c29, a78+c30, a78+c31, a78+c32, a78+c33, a78+c34, a78+c35, a78+c36, a78+c37, a78+c38, a78+c39, a78+c40, a78+c41, a78+c42, a78+c43, a78+c44, a78+c45, a78+c46, a78+c47, a78+c48, a78+c49, a78+c50, a78+c51, a78+c52, a78+c53, a78+c54, a78+c55, a78+c56, a78+c57, a78+c58, a78+c59, a78+c60, a78+c61, a78+c62, a78+c63, a78+c64, a78+c65, a78+c66, a78+c67, a78+c68, a78+c69, a78+c70, a78+c71, a78+c72, a78+c73, a78+c74, a78+c75, a78+c76, a78+c77, a78+c78, a78+c79, a78+c80, a78+c81, a78+c82, a78+c83, a78+c84, a78+c85, a78+c86, a78+c87, a78+c88, a78+c89, a78+c90, a78+c91, a78+c92, a78+c93, a78+c94, a78+c95, a78+c96, a78+c97, a78+c98, a78+c99, a78+c100, a78+c101, a78+c102, a78+c103, a78+c104, a78+c105, a78+c106, a78+c107, a78+c108, a78+c109, a78+c110, a78+c111, a78+c112, a78+c113, a78+c114, a78+c115, a78+c116, a78+c117, a78+c118, a78+c119, a78+c120, a78+c121, a78+c122, a78+c123, a78+c124, a78+c125, a78+c126, a78+c127, a78+c128, a78+c129, a78+c130, a78+c131, a78+c132, a78+c133, a78+c134, a78+c135, a78+c136, a78+c137, a78+c138, a78+c139, a78+c140, a78+c141, a78+c142, a78+c143, a78+c144, a78+c145, a78+c146, a78+c147, a78+c148, a78+c149, a78+c150, a78+c151, a78+c152, a78+c153, a78+c154, a78+c155, a78+c156, a78+c157, a78+c158, a78+c159, a78+c160, a78+c161, a78+c162, a78+c163, a78+c164, a78+c165, a78+c166, a78+c167, a78+c168, a78+c169, a78+c170, a78+c171, a78+c172, a78+c173, a78+c174, a78+c175, a78+c176, a78+c177, a78+c178, a78+c179, a78+c180, a78+c181, a78+c182, a78+c183, a78+c184, a78+c185, a78+c186, a78+c187, a78+c188, a78+c189, a78+c190, a78+c191, a78+c192, a78+c193, a78+c194, a78+c195, a78+c196, a78+c197, a78+c198, a78+c199, a78+c200, a78+c201, a78+c202, a78+c203, a78+c204, a78+c205, a78+c206, a78+c207, a78+c208, a78+c209, a78+c210, a78+c211, a79+c1, a79+c2, a79+c3, a79+c4, a79+c5, a79+c6, a79+c7, a79+c8, a79+c9, a79+c10, a79+c11, a79+c12, a79+c13, a79+c14, a79+c15, a79+c16, a79+c17, a79+c18, a79+c19, a79+c20, a79+c21, a79+c22, a79+c23, a79+c24, a79+c25, a79+c26, a79+c27, a79+c28, a79+c29, a79+c30, a79+c31, a79+c32, a79+c33, a79+c34, a79+c35, a79+c36, a79+c37, a79+c38, a79+c39, a79+c40, a79+c41, a79+c42, a79+c43, a79+c44, a79+c45, a79+c46, a79+c47, a79+c48, a79+c49, a79+c50, a79+c51, a79+c52, a79+c53, a79+c54, a79+c55, a79+c56, a79+c57, a79+c58, a79+c59, a79+c60, a79+c61, a79+c62, a79+c63, a79+c64, a79+c65, a79+c66, a79+c67, a79+c68, a79+c69, a79+c70, a79+c71, a79+c72, a79+c73, a79+c74, a79+c75, a79+c76, a79+c77, a79+c78, a79+c79, a79+c80, a79+c81, a79+c82, a79+c83, a79+c84, a79+c85, a79+c86, a79+c87, a79+c88, a79+c89, a79+c90, a79+c91, a79+c92, a79+c93, a79+c94, a79+c95, a79+c96, a79+c97, a79+c98, a79+c99, a79+c100, a79+c101, a79+c102, a79+c103, a79+c104, a79+c105, a79+c106, a79+c107, a79+c108, a79+c109, a79+c110, a79+c111, a79+c112, a79+c113, a79+c114, a79+c115, a79+c116, a79+c117, a79+c118, a79+c119, a79+c120, a79+c121, a79+c122, a79+c123, a79+c124, a79+c125, a79+c126, a79+c127, a79+c128, a79+c129, a79+c130, a79+c131, a79+c132, a79+c133, a79+c134, a79+c135, a79+c136, a79+c137, a79+c138, a79+c139, a79+c140, a79+c141, a79+c142, a79+c143, a79+c144, a79+c145, a79+c146, a79+c147, a79+c148, a79+c149, a79+c150, a79+c151, a79+c152, a79+c153, a79+c154, a79+c155, a79+c156, a79+c157, a79+c158, a79+c159, a79+c160, a79+c161, a79+c162, a79+c163, a79+c164, a79+c165, a79+c166, a79+c167, a79+c168, a79+c169, a79+c170, a79+c171, a79+c172, a79+c173, a79+c174, a79+c175, a79+c176, a79+c177, a79+c178, a79+c179, a79+c180, a79+c181, a79+c182, a79+c183, a79+c184, a79+c185, a79+c186, a79+c187, a79+c188, a79+c189, a79+c190, a79+c191, a79+c192, a79+c193, a79+c194, a79+c195, a79+c196, a79+c197, a79+c198, a79+c199, a79+c200, a79+c201, a79+c202, a79+c203, a79+c204, a79+c205, a79+c206, a79+c207, a79+c208, a79+c209, a79+c210, a79+c211, a80+c1, a80+c2, a80+c3, a80+c4, a80+c5, a80+c6, a80+c7, a80+c8, a80+c9, a80+c10, a80+c11, a80+c12, a80+c13, a80+c14, a80+c15, a80+c16, a80+c17, a80+c18, a80+c19, a80+c20, a80+c21, a80+c22, a80+c23, a80+c24, a80+c25, a80+c26, a80+c27, a80+c28, a80+c29, a80+c30, a80+c31, a80+c32, a80+c33, a80+c34, a80+c35, a80+c36, a80+c37, a80+c38, a80+c39, a80+c40, a80+c41, a80+c42, a80+c43, a80+c44, a80+c45, a80+c46, a80+c47, a80+c48, a80+c49, a80+c50, a80+c51, a80+c52, a80+c53, a80+c54, a80+c55, a80+c56, a80+c57, a80+c58, a80+c59, a80+c60, a80+c61, a80+c62, a80+c63, a80+c64, a80+c65, a80+c66, a80+c67, a80+c68, a80+c69, a80+c70, a80+c71, a80+c72, a80+c73, a80+c74, a80+c75, a80+c76, a80+c77, a80+c78, a80+c79, a80+c80, a80+c81, a80+c82, a80+c83, a80+c84, a80+c85, a80+c86, a80+c87, a80+c88, a80+c89, a80+c90, a80+c91, a80+c92, a80+c93, a80+c94, a80+c95, a80+c96, a80+c97, a80+c98, a80+c99, a80+c100, a80+c101, a80+c102, a80+c103, a80+c104, a80+c105, a80+c106, a80+c107, a80+c108, a80+c109, a80+c110, a80+c111, a80+c112, a80+c113, a80+c114, a80+c115, a80+c116, a80+c117, a80+c118, a80+c119, a80+c120, a80+c121, a80+c122, a80+c123, a80+c124, a80+c125, a80+c126, a80+c127, a80+c128, a80+c129, a80+c130, a80+c131, a80+c132, a80+c133, a80+c134, a80+c135, a80+c136, a80+c137, a80+c138, a80+c139, a80+c140, a80+c141, a80+c142, a80+c143, a80+c144, a80+c145, a80+c146, a80+c147, a80+c148, a80+c149, a80+c150, a80+c151, a80+c152, a80+c153, a80+c154, a80+c155, a80+c156, a80+c157, a80+c158, a80+c159, a80+c160, a80+c161, a80+c162, a80+c163, a80+c164, a80+c165, a80+c166, a80+c167, a80+c168, a80+c169, a80+c170, a80+c171, a80+c172, a80+c173, a80+c174, a80+c175, a80+c176, a80+c177, a80+c178, a80+c179, a80+c180, a80+c181, a80+c182, a80+c183, a80+c184, a80+c185, a80+c186, a80+c187, a80+c188, a80+c189, a80+c190, a80+c191, a80+c192, a80+c193, a80+c194, a80+c195, a80+c196, a80+c197, a80+c198, a80+c199, a80+c200, a80+c201, a80+c202, a80+c203, a80+c204, a80+c205, a80+c206, a80+c207, a80+c208, a80+c209, a80+c210, a80+c211, a81+c1, a81+c2, a81+c3, a81+c4, a81+c5, a81+c6, a81+c7, a81+c8, a81+c9, a81+c10, a81+c11, a81+c12, a81+c13, a81+c14, a81+c15, a81+c16, a81+c17, a81+c18, a81+c19, a81+c20, a81+c21, a81+c22, a81+c23, a81+c24, a81+c25, a81+c26, a81+c27, a81+c28, a81+c29, a81+c30, a81+c31, a81+c32, a81+c33, a81+c34, a81+c35, a81+c36, a81+c37, a81+c38, a81+c39, a81+c40, a81+c41, a81+c42, a81+c43, a81+c44, a81+c45, a81+c46, a81+c47, a81+c48, a81+c49, a81+c50, a81+c51, a81+c52, a81+c53, a81+c54, a81+c55, a81+c56, a81+c57, a81+c58, a81+c59, a81+c60, a81+c61, a81+c62, a81+c63, a81+c64, a81+c65, a81+c66, a81+c67, a81+c68, a81+c69, a81+c70, a81+c71, a81+c72, a81+c73, a81+c74, a81+c75, a81+c76, a81+c77, a81+c78, a81+c79, a81+c80, a81+c81, a81+c82, a81+c83, a81+c84, a81+c85, a81+c86, a81+c87, a81+c88, a81+c89, a81+c90, a81+c91, a81+c92, a81+c93, a81+c94, a81+c95, a81+c96, a81+c97, a81+c98, a81+c99, a81+c100, a81+c101, a81+c102, a81+c103, a81+c104, a81+c105, a81+c106, a81+c107, a81+c108, a81+c109, a81+c110, a81+c111, a81+c112, a81+c113, a81+c114, a81+c115, a81+c116, a81+c117, a81+c118, a81+c119, a81+c120, a81+c121, a81+c122, a81+c123, a81+c124, a81+c125, a81+c126, a81+c127, a81+c128, a81+c129, a81+c130, a81+c131, a81+c132, a81+c133, a81+c134, a81+c135, a81+c136, a81+c137, a81+c138, a81+c139, a81+c140, a81+c141, a81+c142, a81+c143, a81+c144, a81+c145, a81+c146, a81+c147, a81+c148, a81+c149, a81+c150, a81+c151, a81+c152, a81+c153, a81+c154, a81+c155, a81+c156, a81+c157, a81+c158, a81+c159, a81+c160, a81+c161, a81+c162, a81+c163, a81+c164, a81+c165, a81+c166, a81+c167, a81+c168, a81+c169, a81+c170, a81+c171, a81+c172, a81+c173, a81+c174, a81+c175, a81+c176, a81+c177, a81+c178, a81+c179, a81+c180, a81+c181, a81+c182, a81+c183, a81+c184, a81+c185, a81+c186, a81+c187, a81+c188, a81+c189, a81+c190, a81+c191, a81+c192, a81+c193, a81+c194, a81+c195, a81+c196, a81+c197, a81+c198, a81+c199, a81+c200, a81+c201, a81+c202, a81+c203, a81+c204, a81+c205, a81+c206, a81+c207, a81+c208, a81+c209, a81+c210, a81+c211, a82+c1, a82+c2, a82+c3, a82+c4, a82+c5, a82+c6, a82+c7, a82+c8, a82+c9, a82+c10, a82+c11, a82+c12, a82+c13, a82+c14, a82+c15, a82+c16, a82+c17, a82+c18, a82+c19, a82+c20, a82+c21, a82+c22, a82+c23, a82+c24, a82+c25, a82+c26, a82+c27, a82+c28, a82+c29, a82+c30, a82+c31, a82+c32, a82+c33, a82+c34, a82+c35, a82+c36, a82+c37, a82+c38, a82+c39, a82+c40, a82+c41, a82+c42, a82+c43, a82+c44, a82+c45, a82+c46, a82+c47, a82+c48, a82+c49, a82+c50, a82+c51, a82+c52, a82+c53, a82+c54, a82+c55, a82+c56, a82+c57, a82+c58, a82+c59, a82+c60, a82+c61, a82+c62, a82+c63, a82+c64, a82+c65, a82+c66, a82+c67, a82+c68, a82+c69, a82+c70, a82+c71, a82+c72, a82+c73, a82+c74, a82+c75, a82+c76, a82+c77, a82+c78, a82+c79, a82+c80, a82+c81, a82+c82, a82+c83, a82+c84, a82+c85, a82+c86, a82+c87, a82+c88, a82+c89, a82+c90, a82+c91, a82+c92, a82+c93, a82+c94, a82+c95, a82+c96, a82+c97, a82+c98, a82+c99, a82+c100, a82+c101, a82+c102, a82+c103, a82+c104, a82+c105, a82+c106, a82+c107, a82+c108, a82+c109, a82+c110, a82+c111, a82+c112, a82+c113, a82+c114, a82+c115, a82+c116, a82+c117, a82+c118, a82+c119, a82+c120, a82+c121, a82+c122, a82+c123, a82+c124, a82+c125, a82+c126, a82+c127, a82+c128, a82+c129, a82+c130, a82+c131, a82+c132, a82+c133, a82+c134, a82+c135, a82+c136, a82+c137, a82+c138, a82+c139, a82+c140, a82+c141, a82+c142, a82+c143, a82+c144, a82+c145, a82+c146, a82+c147, a82+c148, a82+c149, a82+c150, a82+c151, a82+c152, a82+c153, a82+c154, a82+c155, a82+c156, a82+c157, a82+c158, a82+c159, a82+c160, a82+c161, a82+c162, a82+c163, a82+c164, a82+c165, a82+c166, a82+c167, a82+c168, a82+c169, a82+c170, a82+c171, a82+c172, a82+c173, a82+c174, a82+c175, a82+c176, a82+c177, a82+c178, a82+c179, a82+c180, a82+c181, a82+c182, a82+c183, a82+c184, a82+c185, a82+c186, a82+c187, a82+c188, a82+c189, a82+c190, a82+c191, a82+c192, a82+c193, a82+c194, a82+c195, a82+c196, a82+c197, a82+c198, a82+c199, a82+c200, a82+c201, a82+c202, a82+c203, a82+c204, a82+c205, a82+c206, a82+c207, a82+c208, a82+c209, a82+c210, a82+c211, a83+c1, a83+c2, a83+c3, a83+c4, a83+c5, a83+c6, a83+c7, a83+c8, a83+c9, a83+c10, a83+c11, a83+c12, a83+c13, a83+c14, a83+c15, a83+c16, a83+c17, a83+c18, a83+c19, a83+c20, a83+c21, a83+c22, a83+c23, a83+c24, a83+c25, a83+c26, a83+c27, a83+c28, a83+c29, a83+c30, a83+c31, a83+c32, a83+c33, a83+c34, a83+c35, a83+c36, a83+c37, a83+c38, a83+c39, a83+c40, a83+c41, a83+c42, a83+c43, a83+c44, a83+c45, a83+c46, a83+c47, a83+c48, a83+c49, a83+c50, a83+c51, a83+c52, a83+c53, a83+c54, a83+c55, a83+c56, a83+c57, a83+c58, a83+c59, a83+c60, a83+c61, a83+c62, a83+c63, a83+c64, a83+c65, a83+c66, a83+c67, a83+c68, a83+c69, a83+c70, a83+c71, a83+c72, a83+c73, a83+c74, a83+c75, a83+c76, a83+c77, a83+c78, a83+c79, a83+c80, a83+c81, a83+c82, a83+c83, a83+c84, a83+c85, a83+c86, a83+c87, a83+c88, a83+c89, a83+c90, a83+c91, a83+c92, a83+c93, a83+c94, a83+c95, a83+c96, a83+c97, a83+c98, a83+c99, a83+c100, a83+c101, a83+c102, a83+c103, a83+c104, a83+c105, a83+c106, a83+c107, a83+c108, a83+c109, a83+c110, a83+c111, a83+c112, a83+c113, a83+c114, a83+c115, a83+c116, a83+c117, a83+c118, a83+c119, a83+c120, a83+c121, a83+c122, a83+c123, a83+c124, a83+c125, a83+c126, a83+c127, a83+c128, a83+c129, a83+c130, a83+c131, a83+c132, a83+c133, a83+c134, a83+c135, a83+c136, a83+c137, a83+c138, a83+c139, a83+c140, a83+c141, a83+c142, a83+c143, a83+c144, a83+c145, a83+c146, a83+c147, a83+c148, a83+c149, a83+c150, a83+c151, a83+c152, a83+c153, a83+c154, a83+c155, a83+c156, a83+c157, a83+c158, a83+c159, a83+c160, a83+c161, a83+c162, a83+c163, a83+c164, a83+c165, a83+c166, a83+c167, a83+c168, a83+c169, a83+c170, a83+c171, a83+c172, a83+c173, a83+c174, a83+c175, a83+c176, a83+c177, a83+c178, a83+c179, a83+c180, a83+c181, a83+c182, a83+c183, a83+c184, a83+c185, a83+c186, a83+c187, a83+c188, a83+c189, a83+c190, a83+c191, a83+c192, a83+c193, a83+c194, a83+c195, a83+c196, a83+c197, a83+c198, a83+c199, a83+c200, a83+c201, a83+c202, a83+c203, a83+c204, a83+c205, a83+c206, a83+c207, a83+c208, a83+c209, a83+c210, a83+c211, a84+c1, a84+c2, a84+c3, a84+c4, a84+c5, a84+c6, a84+c7, a84+c8, a84+c9, a84+c10, a84+c11, a84+c12, a84+c13, a84+c14, a84+c15, a84+c16, a84+c17, a84+c18, a84+c19, a84+c20, a84+c21, a84+c22, a84+c23, a84+c24, a84+c25, a84+c26, a84+c27, a84+c28, a84+c29, a84+c30, a84+c31, a84+c32, a84+c33, a84+c34, a84+c35, a84+c36, a84+c37, a84+c38, a84+c39, a84+c40, a84+c41, a84+c42, a84+c43, a84+c44, a84+c45, a84+c46, a84+c47, a84+c48, a84+c49, a84+c50, a84+c51, a84+c52, a84+c53, a84+c54, a84+c55, a84+c56, a84+c57, a84+c58, a84+c59, a84+c60, a84+c61, a84+c62, a84+c63, a84+c64, a84+c65, a84+c66, a84+c67, a84+c68, a84+c69, a84+c70, a84+c71, a84+c72, a84+c73, a84+c74, a84+c75, a84+c76, a84+c77, a84+c78, a84+c79, a84+c80, a84+c81, a84+c82, a84+c83, a84+c84, a84+c85, a84+c86, a84+c87, a84+c88, a84+c89, a84+c90, a84+c91, a84+c92, a84+c93, a84+c94, a84+c95, a84+c96, a84+c97, a84+c98, a84+c99, a84+c100, a84+c101, a84+c102, a84+c103, a84+c104, a84+c105, a84+c106, a84+c107, a84+c108, a84+c109, a84+c110, a84+c111, a84+c112, a84+c113, a84+c114, a84+c115, a84+c116, a84+c117, a84+c118, a84+c119, a84+c120, a84+c121, a84+c122, a84+c123, a84+c124, a84+c125, a84+c126, a84+c127, a84+c128, a84+c129, a84+c130, a84+c131, a84+c132, a84+c133, a84+c134, a84+c135, a84+c136, a84+c137, a84+c138, a84+c139, a84+c140, a84+c141, a84+c142, a84+c143, a84+c144, a84+c145, a84+c146, a84+c147, a84+c148, a84+c149, a84+c150, a84+c151, a84+c152, a84+c153, a84+c154, a84+c155, a84+c156, a84+c157, a84+c158, a84+c159, a84+c160, a84+c161, a84+c162, a84+c163, a84+c164, a84+c165, a84+c166, a84+c167, a84+c168, a84+c169, a84+c170, a84+c171, a84+c172, a84+c173, a84+c174, a84+c175, a84+c176, a84+c177, a84+c178, a84+c179, a84+c180, a84+c181, a84+c182, a84+c183, a84+c184, a84+c185, a84+c186, a84+c187, a84+c188, a84+c189, a84+c190, a84+c191, a84+c192, a84+c193, a84+c194, a84+c195, a84+c196, a84+c197, a84+c198, a84+c199, a84+c200, a84+c201, a84+c202, a84+c203, a84+c204, a84+c205, a84+c206, a84+c207, a84+c208, a84+c209, a84+c210, a84+c211, a85+c1, a85+c2, a85+c3, a85+c4, a85+c5, a85+c6, a85+c7, a85+c8, a85+c9, a85+c10, a85+c11, a85+c12, a85+c13, a85+c14, a85+c15, a85+c16, a85+c17, a85+c18, a85+c19, a85+c20, a85+c21, a85+c22, a85+c23, a85+c24, a85+c25, a85+c26, a85+c27, a85+c28, a85+c29, a85+c30, a85+c31, a85+c32, a85+c33, a85+c34, a85+c35, a85+c36, a85+c37, a85+c38, a85+c39, a85+c40, a85+c41, a85+c42, a85+c43, a85+c44, a85+c45, a85+c46, a85+c47, a85+c48, a85+c49, a85+c50, a85+c51, a85+c52, a85+c53, a85+c54, a85+c55, a85+c56, a85+c57, a85+c58, a85+c59, a85+c60, a85+c61, a85+c62, a85+c63, a85+c64, a85+c65, a85+c66, a85+c67, a85+c68, a85+c69, a85+c70, a85+c71, a85+c72, a85+c73, a85+c74, a85+c75, a85+c76, a85+c77, a85+c78, a85+c79, a85+c80, a85+c81, a85+c82, a85+c83, a85+c84, a85+c85, a85+c86, a85+c87, a85+c88, a85+c89, a85+c90, a85+c91, a85+c92, a85+c93, a85+c94, a85+c95, a85+c96, a85+c97, a85+c98, a85+c99, a85+c100, a85+c101, a85+c102, a85+c103, a85+c104, a85+c105, a85+c106, a85+c107, a85+c108, a85+c109, a85+c110, a85+c111, a85+c112, a85+c113, a85+c114, a85+c115, a85+c116, a85+c117, a85+c118, a85+c119, a85+c120, a85+c121, a85+c122, a85+c123, a85+c124, a85+c125, a85+c126, a85+c127, a85+c128, a85+c129, a85+c130, a85+c131, a85+c132, a85+c133, a85+c134, a85+c135, a85+c136, a85+c137, a85+c138, a85+c139, a85+c140, a85+c141, a85+c142, a85+c143, a85+c144, a85+c145, a85+c146, a85+c147, a85+c148, a85+c149, a85+c150, a85+c151, a85+c152, a85+c153, a85+c154, a85+c155, a85+c156, a85+c157, a85+c158, a85+c159, a85+c160, a85+c161, a85+c162, a85+c163, a85+c164, a85+c165, a85+c166, a85+c167, a85+c168, a85+c169, a85+c170, a85+c171, a85+c172, a85+c173, a85+c174, a85+c175, a85+c176, a85+c177, a85+c178, a85+c179, a85+c180, a85+c181, a85+c182, a85+c183, a85+c184, a85+c185, a85+c186, a85+c187, a85+c188, a85+c189, a85+c190, a85+c191, a85+c192, a85+c193, a85+c194, a85+c195, a85+c196, a85+c197, a85+c198, a85+c199, a85+c200, a85+c201, a85+c202, a85+c203, a85+c204, a85+c205, a85+c206, a85+c207, a85+c208, a85+c209, a85+c210, a85+c211, a86+c1, a86+c2, a86+c3, a86+c4, a86+c5, a86+c6, a86+c7, a86+c8, a86+c9, a86+c10, a86+c11, a86+c12, a86+c13, a86+c14, a86+c15, a86+c16, a86+c17, a86+c18, a86+c19, a86+c20, a86+c21, a86+c22, a86+c23, a86+c24, a86+c25, a86+c26, a86+c27, a86+c28, a86+c29, a86+c30, a86+c31, a86+c32, a86+c33, a86+c34, a86+c35, a86+c36, a86+c37, a86+c38, a86+c39, a86+c40, a86+c41, a86+c42, a86+c43, a86+c44, a86+c45, a86+c46, a86+c47, a86+c48, a86+c49, a86+c50, a86+c51, a86+c52, a86+c53, a86+c54, a86+c55, a86+c56, a86+c57, a86+c58, a86+c59, a86+c60, a86+c61, a86+c62, a86+c63, a86+c64, a86+c65, a86+c66, a86+c67, a86+c68, a86+c69, a86+c70, a86+c71, a86+c72, a86+c73, a86+c74, a86+c75, a86+c76, a86+c77, a86+c78, a86+c79, a86+c80, a86+c81, a86+c82, a86+c83, a86+c84, a86+c85, a86+c86, a86+c87, a86+c88, a86+c89, a86+c90, a86+c91, a86+c92, a86+c93, a86+c94, a86+c95, a86+c96, a86+c97, a86+c98, a86+c99, a86+c100, a86+c101, a86+c102, a86+c103, a86+c104, a86+c105, a86+c106, a86+c107, a86+c108, a86+c109, a86+c110, a86+c111, a86+c112, a86+c113, a86+c114, a86+c115, a86+c116, a86+c117, a86+c118, a86+c119, a86+c120, a86+c121, a86+c122, a86+c123, a86+c124, a86+c125, a86+c126, a86+c127, a86+c128, a86+c129, a86+c130, a86+c131, a86+c132, a86+c133, a86+c134, a86+c135, a86+c136, a86+c137, a86+c138, a86+c139, a86+c140, a86+c141, a86+c142, a86+c143, a86+c144, a86+c145, a86+c146, a86+c147, a86+c148, a86+c149, a86+c150, a86+c151, a86+c152, a86+c153, a86+c154, a86+c155, a86+c156, a86+c157, a86+c158, a86+c159, a86+c160, a86+c161, a86+c162, a86+c163, a86+c164, a86+c165, a86+c166, a86+c167, a86+c168, a86+c169, a86+c170, a86+c171, a86+c172, a86+c173, a86+c174, a86+c175, a86+c176, a86+c177, a86+c178, a86+c179, a86+c180, a86+c181, a86+c182, a86+c183, a86+c184, a86+c185, a86+c186, a86+c187, a86+c188, a86+c189, a86+c190, a86+c191, a86+c192, a86+c193, a86+c194, a86+c195, a86+c196, a86+c197, a86+c198, a86+c199, a86+c200, a86+c201, a86+c202, a86+c203, a86+c204, a86+c205, a86+c206, a86+c207, a86+c208, a86+c209, a86+c210, a86+c211, a87+c1, a87+c2, a87+c3, a87+c4, a87+c5, a87+c6, a87+c7, a87+c8, a87+c9, a87+c10, a87+c11, a87+c12, a87+c13, a87+c14, a87+c15, a87+c16, a87+c17, a87+c18, a87+c19, a87+c20, a87+c21, a87+c22, a87+c23, a87+c24, a87+c25, a87+c26, a87+c27, a87+c28, a87+c29, a87+c30, a87+c31, a87+c32, a87+c33, a87+c34, a87+c35, a87+c36, a87+c37, a87+c38, a87+c39, a87+c40, a87+c41, a87+c42, a87+c43, a87+c44, a87+c45, a87+c46, a87+c47, a87+c48, a87+c49, a87+c50, a87+c51, a87+c52, a87+c53, a87+c54, a87+c55, a87+c56, a87+c57, a87+c58, a87+c59, a87+c60, a87+c61, a87+c62, a87+c63, a87+c64, a87+c65, a87+c66, a87+c67, a87+c68, a87+c69, a87+c70, a87+c71, a87+c72, a87+c73, a87+c74, a87+c75, a87+c76, a87+c77, a87+c78, a87+c79, a87+c80, a87+c81, a87+c82, a87+c83, a87+c84, a87+c85, a87+c86, a87+c87, a87+c88, a87+c89, a87+c90, a87+c91, a87+c92, a87+c93, a87+c94, a87+c95, a87+c96, a87+c97, a87+c98, a87+c99, a87+c100, a87+c101, a87+c102, a87+c103, a87+c104, a87+c105, a87+c106, a87+c107, a87+c108, a87+c109, a87+c110, a87+c111, a87+c112, a87+c113, a87+c114, a87+c115, a87+c116, a87+c117, a87+c118, a87+c119, a87+c120, a87+c121, a87+c122, a87+c123, a87+c124, a87+c125, a87+c126, a87+c127, a87+c128, a87+c129, a87+c130, a87+c131, a87+c132, a87+c133, a87+c134, a87+c135, a87+c136, a87+c137, a87+c138, a87+c139, a87+c140, a87+c141, a87+c142, a87+c143, a87+c144, a87+c145, a87+c146, a87+c147, a87+c148, a87+c149, a87+c150, a87+c151, a87+c152, a87+c153, a87+c154, a87+c155, a87+c156, a87+c157, a87+c158, a87+c159, a87+c160, a87+c161, a87+c162, a87+c163, a87+c164, a87+c165, a87+c166, a87+c167, a87+c168, a87+c169, a87+c170, a87+c171, a87+c172, a87+c173, a87+c174, a87+c175, a87+c176, a87+c177, a87+c178, a87+c179, a87+c180, a87+c181, a87+c182, a87+c183, a87+c184, a87+c185, a87+c186, a87+c187, a87+c188, a87+c189, a87+c190, a87+c191, a87+c192, a87+c193, a87+c194, a87+c195, a87+c196, a87+c197, a87+c198, a87+c199, a87+c200, a87+c201, a87+c202, a87+c203, a87+c204, a87+c205, a87+c206, a87+c207, a87+c208, a87+c209, a87+c210, a87+c211, a88+c1, a88+c2, a88+c3, a88+c4, a88+c5, a88+c6, a88+c7, a88+c8, a88+c9, a88+c10, a88+c11, a88+c12, a88+c13, a88+c14, a88+c15, a88+c16, a88+c17, a88+c18, a88+c19, a88+c20, a88+c21, a88+c22, a88+c23, a88+c24, a88+c25, a88+c26, a88+c27, a88+c28, a88+c29, a88+c30, a88+c31, a88+c32, a88+c33, a88+c34, a88+c35, a88+c36, a88+c37, a88+c38, a88+c39, a88+c40, a88+c41, a88+c42, a88+c43, a88+c44, a88+c45, a88+c46, a88+c47, a88+c48, a88+c49, a88+c50, a88+c51, a88+c52, a88+c53, a88+c54, a88+c55, a88+c56, a88+c57, a88+c58, a88+c59, a88+c60, a88+c61, a88+c62, a88+c63, a88+c64, a88+c65, a88+c66, a88+c67, a88+c68, a88+c69, a88+c70, a88+c71, a88+c72, a88+c73, a88+c74, a88+c75, a88+c76, a88+c77, a88+c78, a88+c79, a88+c80, a88+c81, a88+c82, a88+c83, a88+c84, a88+c85, a88+c86, a88+c87, a88+c88, a88+c89, a88+c90, a88+c91, a88+c92, a88+c93, a88+c94, a88+c95, a88+c96, a88+c97, a88+c98, a88+c99, a88+c100, a88+c101, a88+c102, a88+c103, a88+c104, a88+c105, a88+c106, a88+c107, a88+c108, a88+c109, a88+c110, a88+c111, a88+c112, a88+c113, a88+c114, a88+c115, a88+c116, a88+c117, a88+c118, a88+c119, a88+c120, a88+c121, a88+c122, a88+c123, a88+c124, a88+c125, a88+c126, a88+c127, a88+c128, a88+c129, a88+c130, a88+c131, a88+c132, a88+c133, a88+c134, a88+c135, a88+c136, a88+c137, a88+c138, a88+c139, a88+c140, a88+c141, a88+c142, a88+c143, a88+c144, a88+c145, a88+c146, a88+c147, a88+c148, a88+c149, a88+c150, a88+c151, a88+c152, a88+c153, a88+c154, a88+c155, a88+c156, a88+c157, a88+c158, a88+c159, a88+c160, a88+c161, a88+c162, a88+c163, a88+c164, a88+c165, a88+c166, a88+c167, a88+c168, a88+c169, a88+c170, a88+c171, a88+c172, a88+c173, a88+c174, a88+c175, a88+c176, a88+c177, a88+c178, a88+c179, a88+c180, a88+c181, a88+c182, a88+c183, a88+c184, a88+c185, a88+c186, a88+c187, a88+c188, a88+c189, a88+c190, a88+c191, a88+c192, a88+c193, a88+c194, a88+c195, a88+c196, a88+c197, a88+c198, a88+c199, a88+c200, a88+c201, a88+c202, a88+c203, a88+c204, a88+c205, a88+c206, a88+c207, a88+c208, a88+c209, a88+c210, a88+c211, a89+c1, a89+c2, a89+c3, a89+c4, a89+c5, a89+c6, a89+c7, a89+c8, a89+c9, a89+c10, a89+c11, a89+c12, a89+c13, a89+c14, a89+c15, a89+c16, a89+c17, a89+c18, a89+c19, a89+c20, a89+c21, a89+c22, a89+c23, a89+c24, a89+c25, a89+c26, a89+c27, a89+c28, a89+c29, a89+c30, a89+c31, a89+c32, a89+c33, a89+c34, a89+c35, a89+c36, a89+c37, a89+c38, a89+c39, a89+c40, a89+c41, a89+c42, a89+c43, a89+c44, a89+c45, a89+c46, a89+c47, a89+c48, a89+c49, a89+c50, a89+c51, a89+c52, a89+c53, a89+c54, a89+c55, a89+c56, a89+c57, a89+c58, a89+c59, a89+c60, a89+c61, a89+c62, a89+c63, a89+c64, a89+c65, a89+c66, a89+c67, a89+c68, a89+c69, a89+c70, a89+c71, a89+c72, a89+c73, a89+c74, a89+c75, a89+c76, a89+c77, a89+c78, a89+c79, a89+c80, a89+c81, a89+c82, a89+c83, a89+c84, a89+c85, a89+c86, a89+c87, a89+c88, a89+c89, a89+c90, a89+c91, a89+c92, a89+c93, a89+c94, a89+c95, a89+c96, a89+c97, a89+c98, a89+c99, a89+c100, a89+c101, a89+c102, a89+c103, a89+c104, a89+c105, a89+c106, a89+c107, a89+c108, a89+c109, a89+c110, a89+c111, a89+c112, a89+c113, a89+c114, a89+c115, a89+c116, a89+c117, a89+c118, a89+c119, a89+c120, a89+c121, a89+c122, a89+c123, a89+c124, a89+c125, a89+c126, a89+c127, a89+c128, a89+c129, a89+c130, a89+c131, a89+c132, a89+c133, a89+c134, a89+c135, a89+c136, a89+c137, a89+c138, a89+c139, a89+c140, a89+c141, a89+c142, a89+c143, a89+c144, a89+c145, a89+c146, a89+c147, a89+c148, a89+c149, a89+c150, a89+c151, a89+c152, a89+c153, a89+c154, a89+c155, a89+c156, a89+c157, a89+c158, a89+c159, a89+c160, a89+c161, a89+c162, a89+c163, a89+c164, a89+c165, a89+c166, a89+c167, a89+c168, a89+c169, a89+c170, a89+c171, a89+c172, a89+c173, a89+c174, a89+c175, a89+c176, a89+c177, a89+c178, a89+c179, a89+c180, a89+c181, a89+c182, a89+c183, a89+c184, a89+c185, a89+c186, a89+c187, a89+c188, a89+c189, a89+c190, a89+c191, a89+c192, a89+c193, a89+c194, a89+c195, a89+c196, a89+c197, a89+c198, a89+c199, a89+c200, a89+c201, a89+c202, a89+c203, a89+c204, a89+c205, a89+c206, a89+c207, a89+c208, a89+c209, a89+c210, a89+c211, a90+c1, a90+c2, a90+c3, a90+c4, a90+c5, a90+c6, a90+c7, a90+c8, a90+c9, a90+c10, a90+c11, a90+c12, a90+c13, a90+c14, a90+c15, a90+c16, a90+c17, a90+c18, a90+c19, a90+c20, a90+c21, a90+c22, a90+c23, a90+c24, a90+c25, a90+c26, a90+c27, a90+c28, a90+c29, a90+c30, a90+c31, a90+c32, a90+c33, a90+c34, a90+c35, a90+c36, a90+c37, a90+c38, a90+c39, a90+c40, a90+c41, a90+c42, a90+c43, a90+c44, a90+c45, a90+c46, a90+c47, a90+c48, a90+c49, a90+c50, a90+c51, a90+c52, a90+c53, a90+c54, a90+c55, a90+c56, a90+c57, a90+c58, a90+c59, a90+c60, a90+c61, a90+c62, a90+c63, a90+c64, a90+c65, a90+c66, a90+c67, a90+c68, a90+c69, a90+c70, a90+c71, a90+c72, a90+c73, a90+c74, a90+c75, a90+c76, a90+c77, a90+c78, a90+c79, a90+c80, a90+c81, a90+c82, a90+c83, a90+c84, a90+c85, a90+c86, a90+c87, a90+c88, a90+c89, a90+c90, a90+c91, a90+c92, a90+c93, a90+c94, a90+c95, a90+c96, a90+c97, a90+c98, a90+c99, a90+c100, a90+c101, a90+c102, a90+c103, a90+c104, a90+c105, a90+c106, a90+c107, a90+c108, a90+c109, a90+c110, a90+c111, a90+c112, a90+c113, a90+c114, a90+c115, a90+c116, a90+c117, a90+c118, a90+c119, a90+c120, a90+c121, a90+c122, a90+c123, a90+c124, a90+c125, a90+c126, a90+c127, a90+c128, a90+c129, a90+c130, a90+c131, a90+c132, a90+c133, a90+c134, a90+c135, a90+c136, a90+c137, a90+c138, a90+c139, a90+c140, a90+c141, a90+c142, a90+c143, a90+c144, a90+c145, a90+c146, a90+c147, a90+c148, a90+c149, a90+c150, a90+c151, a90+c152, a90+c153, a90+c154, a90+c155, a90+c156, a90+c157, a90+c158, a90+c159, a90+c160, a90+c161, a90+c162, a90+c163, a90+c164, a90+c165, a90+c166, a90+c167, a90+c168, a90+c169, a90+c170, a90+c171, a90+c172, a90+c173, a90+c174, a90+c175, a90+c176, a90+c177, a90+c178, a90+c179, a90+c180, a90+c181, a90+c182, a90+c183, a90+c184, a90+c185, a90+c186, a90+c187, a90+c188, a90+c189, a90+c190, a90+c191, a90+c192, a90+c193, a90+c194, a90+c195, a90+c196, a90+c197, a90+c198, a90+c199, a90+c200, a90+c201, a90+c202, a90+c203, a90+c204, a90+c205, a90+c206, a90+c207, a90+c208, a90+c209, a90+c210, a90+c211, a91+c1, a91+c2, a91+c3, a91+c4, a91+c5, a91+c6, a91+c7, a91+c8, a91+c9, a91+c10, a91+c11, a91+c12, a91+c13, a91+c14, a91+c15, a91+c16, a91+c17, a91+c18, a91+c19, a91+c20, a91+c21, a91+c22, a91+c23, a91+c24, a91+c25, a91+c26, a91+c27, a91+c28, a91+c29, a91+c30, a91+c31, a91+c32, a91+c33, a91+c34, a91+c35, a91+c36, a91+c37, a91+c38, a91+c39, a91+c40, a91+c41, a91+c42, a91+c43, a91+c44, a91+c45, a91+c46, a91+c47, a91+c48, a91+c49, a91+c50, a91+c51, a91+c52, a91+c53, a91+c54, a91+c55, a91+c56, a91+c57, a91+c58, a91+c59, a91+c60, a91+c61, a91+c62, a91+c63, a91+c64, a91+c65, a91+c66, a91+c67, a91+c68, a91+c69, a91+c70, a91+c71, a91+c72, a91+c73, a91+c74, a91+c75, a91+c76, a91+c77, a91+c78, a91+c79, a91+c80, a91+c81, a91+c82, a91+c83, a91+c84, a91+c85, a91+c86, a91+c87, a91+c88, a91+c89, a91+c90, a91+c91, a91+c92, a91+c93, a91+c94, a91+c95, a91+c96, a91+c97, a91+c98, a91+c99, a91+c100, a91+c101, a91+c102, a91+c103, a91+c104, a91+c105, a91+c106, a91+c107, a91+c108, a91+c109, a91+c110, a91+c111, a91+c112, a91+c113, a91+c114, a91+c115, a91+c116, a91+c117, a91+c118, a91+c119, a91+c120, a91+c121, a91+c122, a91+c123, a91+c124, a91+c125, a91+c126, a91+c127, a91+c128, a91+c129, a91+c130, a91+c131, a91+c132, a91+c133, a91+c134, a91+c135, a91+c136, a91+c137, a91+c138, a91+c139, a91+c140, a91+c141, a91+c142, a91+c143, a91+c144, a91+c145, a91+c146, a91+c147, a91+c148, a91+c149, a91+c150, a91+c151, a91+c152, a91+c153, a91+c154, a91+c155, a91+c156, a91+c157, a91+c158, a91+c159, a91+c160, a91+c161, a91+c162, a91+c163, a91+c164, a91+c165, a91+c166, a91+c167, a91+c168, a91+c169, a91+c170, a91+c171, a91+c172, a91+c173, a91+c174, a91+c175, a91+c176, a91+c177, a91+c178, a91+c179, a91+c180, a91+c181, a91+c182, a91+c183, a91+c184, a91+c185, a91+c186, a91+c187, a91+c188, a91+c189, a91+c190, a91+c191, a91+c192, a91+c193, a91+c194, a91+c195, a91+c196, a91+c197, a91+c198, a91+c199, a91+c200, a91+c201, a91+c202, a91+c203, a91+c204, a91+c205, a91+c206, a91+c207, a91+c208, a91+c209, a91+c210, a91+c211, a92+c1, a92+c2, a92+c3, a92+c4, a92+c5, a92+c6, a92+c7, a92+c8, a92+c9, a92+c10, a92+c11, a92+c12, a92+c13, a92+c14, a92+c15, a92+c16, a92+c17, a92+c18, a92+c19, a92+c20, a92+c21, a92+c22, a92+c23, a92+c24, a92+c25, a92+c26, a92+c27, a92+c28, a92+c29, a92+c30, a92+c31, a92+c32, a92+c33, a92+c34, a92+c35, a92+c36, a92+c37, a92+c38, a92+c39, a92+c40, a92+c41, a92+c42, a92+c43, a92+c44, a92+c45, a92+c46, a92+c47, a92+c48, a92+c49, a92+c50, a92+c51, a92+c52, a92+c53, a92+c54, a92+c55, a92+c56, a92+c57, a92+c58, a92+c59, a92+c60, a92+c61, a92+c62, a92+c63, a92+c64, a92+c65, a92+c66, a92+c67, a92+c68, a92+c69, a92+c70, a92+c71, a92+c72, a92+c73, a92+c74, a92+c75, a92+c76, a92+c77, a92+c78, a92+c79, a92+c80, a92+c81, a92+c82, a92+c83, a92+c84, a92+c85, a92+c86, a92+c87, a92+c88, a92+c89, a92+c90, a92+c91, a92+c92, a92+c93, a92+c94, a92+c95, a92+c96, a92+c97, a92+c98, a92+c99, a92+c100, a92+c101, a92+c102, a92+c103, a92+c104, a92+c105, a92+c106, a92+c107, a92+c108, a92+c109, a92+c110, a92+c111, a92+c112, a92+c113, a92+c114, a92+c115, a92+c116, a92+c117, a92+c118, a92+c119, a92+c120, a92+c121, a92+c122, a92+c123, a92+c124, a92+c125, a92+c126, a92+c127, a92+c128, a92+c129, a92+c130, a92+c131, a92+c132, a92+c133, a92+c134, a92+c135, a92+c136, a92+c137, a92+c138, a92+c139, a92+c140, a92+c141, a92+c142, a92+c143, a92+c144, a92+c145, a92+c146, a92+c147, a92+c148, a92+c149, a92+c150, a92+c151, a92+c152, a92+c153, a92+c154, a92+c155, a92+c156, a92+c157, a92+c158, a92+c159, a92+c160, a92+c161, a92+c162, a92+c163, a92+c164, a92+c165, a92+c166, a92+c167, a92+c168, a92+c169, a92+c170, a92+c171, a92+c172, a92+c173, a92+c174, a92+c175, a92+c176, a92+c177, a92+c178, a92+c179, a92+c180, a92+c181, a92+c182, a92+c183, a92+c184, a92+c185, a92+c186, a92+c187, a92+c188, a92+c189, a92+c190, a92+c191, a92+c192, a92+c193, a92+c194, a92+c195, a92+c196, a92+c197, a92+c198, a92+c199, a92+c200, a92+c201, a92+c202, a92+c203, a92+c204, a92+c205, a92+c206, a92+c207, a92+c208, a92+c209, a92+c210, a92+c211, a93+c1, a93+c2, a93+c3, a93+c4, a93+c5, a93+c6, a93+c7, a93+c8, a93+c9, a93+c10, a93+c11, a93+c12, a93+c13, a93+c14, a93+c15, a93+c16, a93+c17, a93+c18, a93+c19, a93+c20, a93+c21, a93+c22, a93+c23, a93+c24, a93+c25, a93+c26, a93+c27, a93+c28, a93+c29, a93+c30, a93+c31, a93+c32, a93+c33, a93+c34, a93+c35, a93+c36, a93+c37, a93+c38, a93+c39, a93+c40, a93+c41, a93+c42, a93+c43, a93+c44, a93+c45, a93+c46, a93+c47, a93+c48, a93+c49, a93+c50, a93+c51, a93+c52, a93+c53, a93+c54, a93+c55, a93+c56, a93+c57, a93+c58, a93+c59, a93+c60, a93+c61, a93+c62, a93+c63, a93+c64, a93+c65, a93+c66, a93+c67, a93+c68, a93+c69, a93+c70, a93+c71, a93+c72, a93+c73, a93+c74, a93+c75, a93+c76, a93+c77, a93+c78, a93+c79, a93+c80, a93+c81, a93+c82, a93+c83, a93+c84, a93+c85, a93+c86, a93+c87, a93+c88, a93+c89, a93+c90, a93+c91, a93+c92, a93+c93, a93+c94, a93+c95, a93+c96, a93+c97, a93+c98, a93+c99, a93+c100, a93+c101, a93+c102, a93+c103, a93+c104, a93+c105, a93+c106, a93+c107, a93+c108, a93+c109, a93+c110, a93+c111, a93+c112, a93+c113, a93+c114, a93+c115, a93+c116, a93+c117, a93+c118, a93+c119, a93+c120, a93+c121, a93+c122, a93+c123, a93+c124, a93+c125, a93+c126, a93+c127, a93+c128, a93+c129, a93+c130, a93+c131, a93+c132, a93+c133, a93+c134, a93+c135, a93+c136, a93+c137, a93+c138, a93+c139, a93+c140, a93+c141, a93+c142, a93+c143, a93+c144, a93+c145, a93+c146, a93+c147, a93+c148, a93+c149, a93+c150, a93+c151, a93+c152, a93+c153, a93+c154, a93+c155, a93+c156, a93+c157, a93+c158, a93+c159, a93+c160, a93+c161, a93+c162, a93+c163, a93+c164, a93+c165, a93+c166, a93+c167, a93+c168, a93+c169, a93+c170, a93+c171, a93+c172, a93+c173, a93+c174, a93+c175, a93+c176, a93+c177, a93+c178, a93+c179, a93+c180, a93+c181, a93+c182, a93+c183, a93+c184, a93+c185, a93+c186, a93+c187, a93+c188, a93+c189, a93+c190, a93+c191, a93+c192, a93+c193, a93+c194, a93+c195, a93+c196, a93+c197, a93+c198, a93+c199, a93+c200, a93+c201, a93+c202, a93+c203, a93+c204, a93+c205, a93+c206, a93+c207, a93+c208, a93+c209, a93+c210, a93+c211, a94+c1, a94+c2, a94+c3, a94+c4, a94+c5, a94+c6, a94+c7, a94+c8, a94+c9, a94+c10, a94+c11, a94+c12, a94+c13, a94+c14, a94+c15, a94+c16, a94+c17, a94+c18, a94+c19, a94+c20, a94+c21, a94+c22, a94+c23, a94+c24, a94+c25, a94+c26, a94+c27, a94+c28, a94+c29, a94+c30, a94+c31, a94+c32, a94+c33, a94+c34, a94+c35, a94+c36, a94+c37, a94+c38, a94+c39, a94+c40, a94+c41, a94+c42, a94+c43, a94+c44, a94+c45, a94+c46, a94+c47, a94+c48, a94+c49, a94+c50, a94+c51, a94+c52, a94+c53, a94+c54, a94+c55, a94+c56, a94+c57, a94+c58, a94+c59, a94+c60, a94+c61, a94+c62, a94+c63, a94+c64, a94+c65, a94+c66, a94+c67, a94+c68, a94+c69, a94+c70, a94+c71, a94+c72, a94+c73, a94+c74, a94+c75, a94+c76, a94+c77, a94+c78, a94+c79, a94+c80, a94+c81, a94+c82, a94+c83, a94+c84, a94+c85, a94+c86, a94+c87, a94+c88, a94+c89, a94+c90, a94+c91, a94+c92, a94+c93, a94+c94, a94+c95, a94+c96, a94+c97, a94+c98, a94+c99, a94+c100, a94+c101, a94+c102, a94+c103, a94+c104, a94+c105, a94+c106, a94+c107, a94+c108, a94+c109, a94+c110, a94+c111, a94+c112, a94+c113, a94+c114, a94+c115, a94+c116, a94+c117, a94+c118, a94+c119, a94+c120, a94+c121, a94+c122, a94+c123, a94+c124, a94+c125, a94+c126, a94+c127, a94+c128, a94+c129, a94+c130, a94+c131, a94+c132, a94+c133, a94+c134, a94+c135, a94+c136, a94+c137, a94+c138, a94+c139, a94+c140, a94+c141, a94+c142, a94+c143, a94+c144, a94+c145, a94+c146, a94+c147, a94+c148, a94+c149, a94+c150, a94+c151, a94+c152, a94+c153, a94+c154, a94+c155, a94+c156, a94+c157, a94+c158, a94+c159, a94+c160, a94+c161, a94+c162, a94+c163, a94+c164, a94+c165, a94+c166, a94+c167, a94+c168, a94+c169, a94+c170, a94+c171, a94+c172, a94+c173, a94+c174, a94+c175, a94+c176, a94+c177, a94+c178, a94+c179, a94+c180, a94+c181, a94+c182, a94+c183, a94+c184, a94+c185, a94+c186, a94+c187, a94+c188, a94+c189, a94+c190, a94+c191, a94+c192, a94+c193, a94+c194, a94+c195, a94+c196, a94+c197, a94+c198, a94+c199, a94+c200, a94+c201, a94+c202, a94+c203, a94+c204, a94+c205, a94+c206, a94+c207, a94+c208, a94+c209, a94+c210, a94+c211, a95+c1, a95+c2, a95+c3, a95+c4, a95+c5, a95+c6, a95+c7, a95+c8, a95+c9, a95+c10, a95+c11, a95+c12, a95+c13, a95+c14, a95+c15, a95+c16, a95+c17, a95+c18, a95+c19, a95+c20, a95+c21, a95+c22, a95+c23, a95+c24, a95+c25, a95+c26, a95+c27, a95+c28, a95+c29, a95+c30, a95+c31, a95+c32, a95+c33, a95+c34, a95+c35, a95+c36, a95+c37, a95+c38, a95+c39, a95+c40, a95+c41, a95+c42, a95+c43, a95+c44, a95+c45, a95+c46, a95+c47, a95+c48, a95+c49, a95+c50, a95+c51, a95+c52, a95+c53, a95+c54, a95+c55, a95+c56, a95+c57, a95+c58, a95+c59, a95+c60, a95+c61, a95+c62, a95+c63, a95+c64, a95+c65, a95+c66, a95+c67, a95+c68, a95+c69, a95+c70, a95+c71, a95+c72, a95+c73, a95+c74, a95+c75, a95+c76, a95+c77, a95+c78, a95+c79, a95+c80, a95+c81, a95+c82, a95+c83, a95+c84, a95+c85, a95+c86, a95+c87, a95+c88, a95+c89, a95+c90, a95+c91, a95+c92, a95+c93, a95+c94, a95+c95, a95+c96, a95+c97, a95+c98, a95+c99, a95+c100, a95+c101, a95+c102, a95+c103, a95+c104, a95+c105, a95+c106, a95+c107, a95+c108, a95+c109, a95+c110, a95+c111, a95+c112, a95+c113, a95+c114, a95+c115, a95+c116, a95+c117, a95+c118, a95+c119, a95+c120, a95+c121, a95+c122, a95+c123, a95+c124, a95+c125, a95+c126, a95+c127, a95+c128, a95+c129, a95+c130, a95+c131, a95+c132, a95+c133, a95+c134, a95+c135, a95+c136, a95+c137, a95+c138, a95+c139, a95+c140, a95+c141, a95+c142, a95+c143, a95+c144, a95+c145, a95+c146, a95+c147, a95+c148, a95+c149, a95+c150, a95+c151, a95+c152, a95+c153, a95+c154, a95+c155, a95+c156, a95+c157, a95+c158, a95+c159, a95+c160, a95+c161, a95+c162, a95+c163, a95+c164, a95+c165, a95+c166, a95+c167, a95+c168, a95+c169, a95+c170, a95+c171, a95+c172, a95+c173, a95+c174, a95+c175, a95+c176, a95+c177, a95+c178, a95+c179, a95+c180, a95+c181, a95+c182, a95+c183, a95+c184, a95+c185, a95+c186, a95+c187, a95+c188, a95+c189, a95+c190, a95+c191, a95+c192, a95+c193, a95+c194, a95+c195, a95+c196, a95+c197, a95+c198, a95+c199, a95+c200, a95+c201, a95+c202, a95+c203, a95+c204, a95+c205, a95+c206, a95+c207, a95+c208, a95+c209, a95+c210, a95+c211, a96+c1, a96+c2, a96+c3, a96+c4, a96+c5, a96+c6, a96+c7, a96+c8, a96+c9, a96+c10, a96+c11, a96+c12, a96+c13, a96+c14, a96+c15, a96+c16, a96+c17, a96+c18, a96+c19, a96+c20, a96+c21, a96+c22, a96+c23, a96+c24, a96+c25, a96+c26, a96+c27, a96+c28, a96+c29, a96+c30, a96+c31, a96+c32, a96+c33, a96+c34, a96+c35, a96+c36, a96+c37, a96+c38, a96+c39, a96+c40, a96+c41, a96+c42, a96+c43, a96+c44, a96+c45, a96+c46, a96+c47, a96+c48, a96+c49, a96+c50, a96+c51, a96+c52, a96+c53, a96+c54, a96+c55, a96+c56, a96+c57, a96+c58, a96+c59, a96+c60, a96+c61, a96+c62, a96+c63, a96+c64, a96+c65, a96+c66, a96+c67, a96+c68, a96+c69, a96+c70, a96+c71, a96+c72, a96+c73, a96+c74, a96+c75, a96+c76, a96+c77, a96+c78, a96+c79, a96+c80, a96+c81, a96+c82, a96+c83, a96+c84, a96+c85, a96+c86, a96+c87, a96+c88, a96+c89, a96+c90, a96+c91, a96+c92, a96+c93, a96+c94, a96+c95, a96+c96, a96+c97, a96+c98, a96+c99, a96+c100, a96+c101, a96+c102, a96+c103, a96+c104, a96+c105, a96+c106, a96+c107, a96+c108, a96+c109, a96+c110, a96+c111, a96+c112, a96+c113, a96+c114, a96+c115, a96+c116, a96+c117, a96+c118, a96+c119, a96+c120, a96+c121, a96+c122, a96+c123, a96+c124, a96+c125, a96+c126, a96+c127, a96+c128, a96+c129, a96+c130, a96+c131, a96+c132, a96+c133, a96+c134, a96+c135, a96+c136, a96+c137, a96+c138, a96+c139, a96+c140, a96+c141, a96+c142, a96+c143, a96+c144, a96+c145, a96+c146, a96+c147, a96+c148, a96+c149, a96+c150, a96+c151, a96+c152, a96+c153, a96+c154, a96+c155, a96+c156, a96+c157, a96+c158, a96+c159, a96+c160, a96+c161, a96+c162, a96+c163, a96+c164, a96+c165, a96+c166, a96+c167, a96+c168, a96+c169, a96+c170, a96+c171, a96+c172, a96+c173, a96+c174, a96+c175, a96+c176, a96+c177, a96+c178, a96+c179, a96+c180, a96+c181, a96+c182, a96+c183, a96+c184, a96+c185, a96+c186, a96+c187, a96+c188, a96+c189, a96+c190, a96+c191, a96+c192, a96+c193, a96+c194, a96+c195, a96+c196, a96+c197, a96+c198, a96+c199, a96+c200, a96+c201, a96+c202, a96+c203, a96+c204, a96+c205, a96+c206, a96+c207, a96+c208, a96+c209, a96+c210, a96+c211, a97+c1, a97+c2, a97+c3, a97+c4, a97+c5, a97+c6, a97+c7, a97+c8, a97+c9, a97+c10, a97+c11, a97+c12, a97+c13, a97+c14, a97+c15, a97+c16, a97+c17, a97+c18, a97+c19, a97+c20, a97+c21, a97+c22, a97+c23, a97+c24, a97+c25, a97+c26, a97+c27, a97+c28, a97+c29, a97+c30, a97+c31, a97+c32, a97+c33, a97+c34, a97+c35, a97+c36, a97+c37, a97+c38, a97+c39, a97+c40, a97+c41, a97+c42, a97+c43, a97+c44, a97+c45, a97+c46, a97+c47, a97+c48, a97+c49, a97+c50, a97+c51, a97+c52, a97+c53, a97+c54, a97+c55, a97+c56, a97+c57, a97+c58, a97+c59, a97+c60, a97+c61, a97+c62, a97+c63, a97+c64, a97+c65, a97+c66, a97+c67, a97+c68, a97+c69, a97+c70, a97+c71, a97+c72, a97+c73, a97+c74, a97+c75, a97+c76, a97+c77, a97+c78, a97+c79, a97+c80, a97+c81, a97+c82, a97+c83, a97+c84, a97+c85, a97+c86, a97+c87, a97+c88, a97+c89, a97+c90, a97+c91, a97+c92, a97+c93, a97+c94, a97+c95, a97+c96, a97+c97, a97+c98, a97+c99, a97+c100, a97+c101, a97+c102, a97+c103, a97+c104, a97+c105, a97+c106, a97+c107, a97+c108, a97+c109, a97+c110, a97+c111, a97+c112, a97+c113, a97+c114, a97+c115, a97+c116, a97+c117, a97+c118, a97+c119, a97+c120, a97+c121, a97+c122, a97+c123, a97+c124, a97+c125, a97+c126, a97+c127, a97+c128, a97+c129, a97+c130, a97+c131, a97+c132, a97+c133, a97+c134, a97+c135, a97+c136, a97+c137, a97+c138, a97+c139, a97+c140, a97+c141, a97+c142, a97+c143, a97+c144, a97+c145, a97+c146, a97+c147, a97+c148, a97+c149, a97+c150, a97+c151, a97+c152, a97+c153, a97+c154, a97+c155, a97+c156, a97+c157, a97+c158, a97+c159, a97+c160, a97+c161, a97+c162, a97+c163, a97+c164, a97+c165, a97+c166, a97+c167, a97+c168, a97+c169, a97+c170, a97+c171, a97+c172, a97+c173, a97+c174, a97+c175, a97+c176, a97+c177, a97+c178, a97+c179, a97+c180, a97+c181, a97+c182, a97+c183, a97+c184, a97+c185, a97+c186, a97+c187, a97+c188, a97+c189, a97+c190, a97+c191, a97+c192, a97+c193, a97+c194, a97+c195, a97+c196, a97+c197, a97+c198, a97+c199, a97+c200, a97+c201, a97+c202, a97+c203, a97+c204, a97+c205, a97+c206, a97+c207, a97+c208, a97+c209, a97+c210, a97+c211, a98+c1, a98+c2, a98+c3, a98+c4, a98+c5, a98+c6, a98+c7, a98+c8, a98+c9, a98+c10, a98+c11, a98+c12, a98+c13, a98+c14, a98+c15, a98+c16, a98+c17, a98+c18, a98+c19, a98+c20, a98+c21, a98+c22, a98+c23, a98+c24, a98+c25, a98+c26, a98+c27, a98+c28, a98+c29, a98+c30, a98+c31, a98+c32, a98+c33, a98+c34, a98+c35, a98+c36, a98+c37, a98+c38, a98+c39, a98+c40, a98+c41, a98+c42, a98+c43, a98+c44, a98+c45, a98+c46, a98+c47, a98+c48, a98+c49, a98+c50, a98+c51, a98+c52, a98+c53, a98+c54, a98+c55, a98+c56, a98+c57, a98+c58, a98+c59, a98+c60, a98+c61, a98+c62, a98+c63, a98+c64, a98+c65, a98+c66, a98+c67, a98+c68, a98+c69, a98+c70, a98+c71, a98+c72, a98+c73, a98+c74, a98+c75, a98+c76, a98+c77, a98+c78, a98+c79, a98+c80, a98+c81, a98+c82, a98+c83, a98+c84, a98+c85, a98+c86, a98+c87, a98+c88, a98+c89, a98+c90, a98+c91, a98+c92, a98+c93, a98+c94, a98+c95, a98+c96, a98+c97, a98+c98, a98+c99, a98+c100, a98+c101, a98+c102, a98+c103, a98+c104, a98+c105, a98+c106, a98+c107, a98+c108, a98+c109, a98+c110, a98+c111, a98+c112, a98+c113, a98+c114, a98+c115, a98+c116, a98+c117, a98+c118, a98+c119, a98+c120, a98+c121, a98+c122, a98+c123, a98+c124, a98+c125, a98+c126, a98+c127, a98+c128, a98+c129, a98+c130, a98+c131, a98+c132, a98+c133, a98+c134, a98+c135, a98+c136, a98+c137, a98+c138, a98+c139, a98+c140, a98+c141, a98+c142, a98+c143, a98+c144, a98+c145, a98+c146, a98+c147, a98+c148, a98+c149, a98+c150, a98+c151, a98+c152, a98+c153, a98+c154, a98+c155, a98+c156, a98+c157, a98+c158, a98+c159, a98+c160, a98+c161, a98+c162, a98+c163, a98+c164, a98+c165, a98+c166, a98+c167, a98+c168, a98+c169, a98+c170, a98+c171, a98+c172, a98+c173, a98+c174, a98+c175, a98+c176, a98+c177, a98+c178, a98+c179, a98+c180, a98+c181, a98+c182, a98+c183, a98+c184, a98+c185, a98+c186, a98+c187, a98+c188, a98+c189, a98+c190, a98+c191, a98+c192, a98+c193, a98+c194, a98+c195, a98+c196, a98+c197, a98+c198, a98+c199, a98+c200, a98+c201, a98+c202, a98+c203, a98+c204, a98+c205, a98+c206, a98+c207, a98+c208, a98+c209, a98+c210, a98+c211, a99+c1, a99+c2, a99+c3, a99+c4, a99+c5, a99+c6, a99+c7, a99+c8, a99+c9, a99+c10, a99+c11, a99+c12, a99+c13, a99+c14, a99+c15, a99+c16, a99+c17, a99+c18, a99+c19, a99+c20, a99+c21, a99+c22, a99+c23, a99+c24, a99+c25, a99+c26, a99+c27, a99+c28, a99+c29, a99+c30, a99+c31, a99+c32, a99+c33, a99+c34, a99+c35, a99+c36, a99+c37, a99+c38, a99+c39, a99+c40, a99+c41, a99+c42, a99+c43, a99+c44, a99+c45, a99+c46, a99+c47, a99+c48, a99+c49, a99+c50, a99+c51, a99+c52, a99+c53, a99+c54, a99+c55, a99+c56, a99+c57, a99+c58, a99+c59, a99+c60, a99+c61, a99+c62, a99+c63, a99+c64, a99+c65, a99+c66, a99+c67, a99+c68, a99+c69, a99+c70, a99+c71, a99+c72, a99+c73, a99+c74, a99+c75, a99+c76, a99+c77, a99+c78, a99+c79, a99+c80, a99+c81, a99+c82, a99+c83, a99+c84, a99+c85, a99+c86, a99+c87, a99+c88, a99+c89, a99+c90, a99+c91, a99+c92, a99+c93, a99+c94, a99+c95, a99+c96, a99+c97, a99+c98, a99+c99, a99+c100, a99+c101, a99+c102, a99+c103, a99+c104, a99+c105, a99+c106, a99+c107, a99+c108, a99+c109, a99+c110, a99+c111, a99+c112, a99+c113, a99+c114, a99+c115, a99+c116, a99+c117, a99+c118, a99+c119, a99+c120, a99+c121, a99+c122, a99+c123, a99+c124, a99+c125, a99+c126, a99+c127, a99+c128, a99+c129, a99+c130, a99+c131, a99+c132, a99+c133, a99+c134, a99+c135, a99+c136, a99+c137, a99+c138, a99+c139, a99+c140, a99+c141, a99+c142, a99+c143, a99+c144, a99+c145, a99+c146, a99+c147, a99+c148, a99+c149, a99+c150, a99+c151, a99+c152, a99+c153, a99+c154, a99+c155, a99+c156, a99+c157, a99+c158, a99+c159, a99+c160, a99+c161, a99+c162, a99+c163, a99+c164, a99+c165, a99+c166, a99+c167, a99+c168, a99+c169, a99+c170, a99+c171, a99+c172, a99+c173, a99+c174, a99+c175, a99+c176, a99+c177, a99+c178, a99+c179, a99+c180, a99+c181, a99+c182, a99+c183, a99+c184, a99+c185, a99+c186, a99+c187, a99+c188, a99+c189, a99+c190, a99+c191, a99+c192, a99+c193, a99+c194, a99+c195, a99+c196, a99+c197, a99+c198, a99+c199, a99+c200, a99+c201, a99+c202, a99+c203, a99+c204, a99+c205, a99+c206, a99+c207, a99+c208, a99+c209, a99+c210, a99+c211, a100+c1, a100+c2, a100+c3, a100+c4, a100+c5, a100+c6, a100+c7, a100+c8, a100+c9, a100+c10, a100+c11, a100+c12, a100+c13, a100+c14, a100+c15, a100+c16, a100+c17, a100+c18, a100+c19, a100+c20, a100+c21, a100+c22, a100+c23, a100+c24, a100+c25, a100+c26, a100+c27, a100+c28, a100+c29, a100+c30, a100+c31, a100+c32, a100+c33, a100+c34, a100+c35, a100+c36, a100+c37, a100+c38, a100+c39, a100+c40, a100+c41, a100+c42, a100+c43, a100+c44, a100+c45, a100+c46, a100+c47, a100+c48, a100+c49, a100+c50, a100+c51, a100+c52, a100+c53, a100+c54, a100+c55, a100+c56, a100+c57, a100+c58, a100+c59, a100+c60, a100+c61, a100+c62, a100+c63, a100+c64, a100+c65, a100+c66, a100+c67, a100+c68, a100+c69, a100+c70, a100+c71, a100+c72, a100+c73, a100+c74, a100+c75, a100+c76, a100+c77, a100+c78, a100+c79, a100+c80, a100+c81, a100+c82, a100+c83, a100+c84, a100+c85, a100+c86, a100+c87, a100+c88, a100+c89, a100+c90, a100+c91, a100+c92, a100+c93, a100+c94, a100+c95, a100+c96, a100+c97, a100+c98, a100+c99, a100+c100, a100+c101, a100+c102, a100+c103, a100+c104, a100+c105, a100+c106, a100+c107, a100+c108, a100+c109, a100+c110, a100+c111, a100+c112, a100+c113, a100+c114, a100+c115, a100+c116, a100+c117, a100+c118, a100+c119, a100+c120, a100+c121, a100+c122, a100+c123, a100+c124, a100+c125, a100+c126, a100+c127, a100+c128, a100+c129, a100+c130, a100+c131, a100+c132, a100+c133, a100+c134, a100+c135, a100+c136, a100+c137, a100+c138, a100+c139, a100+c140, a100+c141, a100+c142, a100+c143, a100+c144, a100+c145, a100+c146, a100+c147, a100+c148, a100+c149, a100+c150, a100+c151, a100+c152, a100+c153, a100+c154, a100+c155, a100+c156, a100+c157, a100+c158, a100+c159, a100+c160, a100+c161, a100+c162, a100+c163, a100+c164, a100+c165, a100+c166, a100+c167, a100+c168, a100+c169, a100+c170, a100+c171, a100+c172, a100+c173, a100+c174, a100+c175, a100+c176, a100+c177, a100+c178, a100+c179, a100+c180, a100+c181, a100+c182, a100+c183, a100+c184, a100+c185, a100+c186, a100+c187, a100+c188, a100+c189, a100+c190, a100+c191, a100+c192, a100+c193, a100+c194, a100+c195, a100+c196, a100+c197, a100+c198, a100+c199, a100+c200, a100+c201, a100+c202, a100+c203, a100+c204, a100+c205, a100+c206, a100+c207, a100+c208, a100+c209, a100+c210, a100+c211, a101+c1, a101+c2, a101+c3, a101+c4, a101+c5, a101+c6, a101+c7, a101+c8, a101+c9, a101+c10, a101+c11, a101+c12, a101+c13, a101+c14, a101+c15, a101+c16, a101+c17, a101+c18, a101+c19, a101+c20, a101+c21, a101+c22, a101+c23, a101+c24, a101+c25, a101+c26, a101+c27, a101+c28, a101+c29, a101+c30, a101+c31, a101+c32, a101+c33, a101+c34, a101+c35, a101+c36, a101+c37, a101+c38, a101+c39, a101+c40, a101+c41, a101+c42, a101+c43, a101+c44, a101+c45, a101+c46, a101+c47, a101+c48, a101+c49, a101+c50, a101+c51, a101+c52, a101+c53, a101+c54, a101+c55, a101+c56, a101+c57, a101+c58, a101+c59, a101+c60, a101+c61, a101+c62, a101+c63, a101+c64, a101+c65, a101+c66, a101+c67, a101+c68, a101+c69, a101+c70, a101+c71, a101+c72, a101+c73, a101+c74, a101+c75, a101+c76, a101+c77, a101+c78, a101+c79, a101+c80, a101+c81, a101+c82, a101+c83, a101+c84, a101+c85, a101+c86, a101+c87, a101+c88, a101+c89, a101+c90, a101+c91, a101+c92, a101+c93, a101+c94, a101+c95, a101+c96, a101+c97, a101+c98, a101+c99, a101+c100, a101+c101, a101+c102, a101+c103, a101+c104, a101+c105, a101+c106, a101+c107, a101+c108, a101+c109, a101+c110, a101+c111, a101+c112, a101+c113, a101+c114, a101+c115, a101+c116, a101+c117, a101+c118, a101+c119, a101+c120, a101+c121, a101+c122, a101+c123, a101+c124, a101+c125, a101+c126, a101+c127, a101+c128, a101+c129, a101+c130, a101+c131, a101+c132, a101+c133, a101+c134, a101+c135, a101+c136, a101+c137, a101+c138, a101+c139, a101+c140, a101+c141, a101+c142, a101+c143, a101+c144, a101+c145, a101+c146, a101+c147, a101+c148, a101+c149, a101+c150, a101+c151, a101+c152, a101+c153, a101+c154, a101+c155, a101+c156, a101+c157, a101+c158, a101+c159, a101+c160, a101+c161, a101+c162, a101+c163, a101+c164, a101+c165, a101+c166, a101+c167, a101+c168, a101+c169, a101+c170, a101+c171, a101+c172, a101+c173, a101+c174, a101+c175, a101+c176, a101+c177, a101+c178, a101+c179, a101+c180, a101+c181, a101+c182, a101+c183, a101+c184, a101+c185, a101+c186, a101+c187, a101+c188, a101+c189, a101+c190, a101+c191, a101+c192, a101+c193, a101+c194, a101+c195, a101+c196, a101+c197, a101+c198, a101+c199, a101+c200, a101+c201, a101+c202, a101+c203, a101+c204, a101+c205, a101+c206, a101+c207, a101+c208, a101+c209, a101+c210, a101+c211, a102+c1, a102+c2, a102+c3, a102+c4, a102+c5, a102+c6, a102+c7, a102+c8, a102+c9, a102+c10, a102+c11, a102+c12, a102+c13, a102+c14, a102+c15, a102+c16, a102+c17, a102+c18, a102+c19, a102+c20, a102+c21, a102+c22, a102+c23, a102+c24, a102+c25, a102+c26, a102+c27, a102+c28, a102+c29, a102+c30, a102+c31, a102+c32, a102+c33, a102+c34, a102+c35, a102+c36, a102+c37, a102+c38, a102+c39, a102+c40, a102+c41, a102+c42, a102+c43, a102+c44, a102+c45, a102+c46, a102+c47, a102+c48, a102+c49, a102+c50, a102+c51, a102+c52, a102+c53, a102+c54, a102+c55, a102+c56, a102+c57, a102+c58, a102+c59, a102+c60, a102+c61, a102+c62, a102+c63, a102+c64, a102+c65, a102+c66, a102+c67, a102+c68, a102+c69, a102+c70, a102+c71, a102+c72, a102+c73, a102+c74, a102+c75, a102+c76, a102+c77, a102+c78, a102+c79, a102+c80, a102+c81, a102+c82, a102+c83, a102+c84, a102+c85, a102+c86, a102+c87, a102+c88, a102+c89, a102+c90, a102+c91, a102+c92, a102+c93, a102+c94, a102+c95, a102+c96, a102+c97, a102+c98, a102+c99, a102+c100, a102+c101, a102+c102, a102+c103, a102+c104, a102+c105, a102+c106, a102+c107, a102+c108, a102+c109, a102+c110, a102+c111, a102+c112, a102+c113, a102+c114, a102+c115, a102+c116, a102+c117, a102+c118, a102+c119, a102+c120, a102+c121, a102+c122, a102+c123, a102+c124, a102+c125, a102+c126, a102+c127, a102+c128, a102+c129, a102+c130, a102+c131, a102+c132, a102+c133, a102+c134, a102+c135, a102+c136, a102+c137, a102+c138, a102+c139, a102+c140, a102+c141, a102+c142, a102+c143, a102+c144, a102+c145, a102+c146, a102+c147, a102+c148, a102+c149, a102+c150, a102+c151, a102+c152, a102+c153, a102+c154, a102+c155, a102+c156, a102+c157, a102+c158, a102+c159, a102+c160, a102+c161, a102+c162, a102+c163, a102+c164, a102+c165, a102+c166, a102+c167, a102+c168, a102+c169, a102+c170, a102+c171, a102+c172, a102+c173, a102+c174, a102+c175, a102+c176, a102+c177, a102+c178, a102+c179, a102+c180, a102+c181, a102+c182, a102+c183, a102+c184, a102+c185, a102+c186, a102+c187, a102+c188, a102+c189, a102+c190, a102+c191, a102+c192, a102+c193, a102+c194, a102+c195, a102+c196, a102+c197, a102+c198, a102+c199, a102+c200, a102+c201, a102+c202, a102+c203, a102+c204, a102+c205, a102+c206, a102+c207, a102+c208, a102+c209, a102+c210, a102+c211, a103+c1, a103+c2, a103+c3, a103+c4, a103+c5, a103+c6, a103+c7, a103+c8, a103+c9, a103+c10, a103+c11, a103+c12, a103+c13, a103+c14, a103+c15, a103+c16, a103+c17, a103+c18, a103+c19, a103+c20, a103+c21, a103+c22, a103+c23, a103+c24, a103+c25, a103+c26, a103+c27, a103+c28, a103+c29, a103+c30, a103+c31, a103+c32, a103+c33, a103+c34, a103+c35, a103+c36, a103+c37, a103+c38, a103+c39, a103+c40, a103+c41, a103+c42, a103+c43, a103+c44, a103+c45, a103+c46, a103+c47, a103+c48, a103+c49, a103+c50, a103+c51, a103+c52, a103+c53, a103+c54, a103+c55, a103+c56, a103+c57, a103+c58, a103+c59, a103+c60, a103+c61, a103+c62, a103+c63, a103+c64, a103+c65, a103+c66, a103+c67, a103+c68, a103+c69, a103+c70, a103+c71, a103+c72, a103+c73, a103+c74, a103+c75, a103+c76, a103+c77, a103+c78, a103+c79, a103+c80, a103+c81, a103+c82, a103+c83, a103+c84, a103+c85, a103+c86, a103+c87, a103+c88, a103+c89, a103+c90, a103+c91, a103+c92, a103+c93, a103+c94, a103+c95, a103+c96, a103+c97, a103+c98, a103+c99, a103+c100, a103+c101, a103+c102, a103+c103, a103+c104, a103+c105, a103+c106, a103+c107, a103+c108, a103+c109, a103+c110, a103+c111, a103+c112, a103+c113, a103+c114, a103+c115, a103+c116, a103+c117, a103+c118, a103+c119, a103+c120, a103+c121, a103+c122, a103+c123, a103+c124, a103+c125, a103+c126, a103+c127, a103+c128, a103+c129, a103+c130, a103+c131, a103+c132, a103+c133, a103+c134, a103+c135, a103+c136, a103+c137, a103+c138, a103+c139, a103+c140, a103+c141, a103+c142, a103+c143, a103+c144, a103+c145, a103+c146, a103+c147, a103+c148, a103+c149, a103+c150, a103+c151, a103+c152, a103+c153, a103+c154, a103+c155, a103+c156, a103+c157, a103+c158, a103+c159, a103+c160, a103+c161, a103+c162, a103+c163, a103+c164, a103+c165, a103+c166, a103+c167, a103+c168, a103+c169, a103+c170, a103+c171, a103+c172, a103+c173, a103+c174, a103+c175, a103+c176, a103+c177, a103+c178, a103+c179, a103+c180, a103+c181, a103+c182, a103+c183, a103+c184, a103+c185, a103+c186, a103+c187, a103+c188, a103+c189, a103+c190, a103+c191, a103+c192, a103+c193, a103+c194, a103+c195, a103+c196, a103+c197, a103+c198, a103+c199, a103+c200, a103+c201, a103+c202, a103+c203, a103+c204, a103+c205, a103+c206, a103+c207, a103+c208, a103+c209, a103+c210, a103+c211, a104+c1, a104+c2, a104+c3, a104+c4, a104+c5, a104+c6, a104+c7, a104+c8, a104+c9, a104+c10, a104+c11, a104+c12, a104+c13, a104+c14, a104+c15, a104+c16, a104+c17, a104+c18, a104+c19, a104+c20, a104+c21, a104+c22, a104+c23, a104+c24, a104+c25, a104+c26, a104+c27, a104+c28, a104+c29, a104+c30, a104+c31, a104+c32, a104+c33, a104+c34, a104+c35, a104+c36, a104+c37, a104+c38, a104+c39, a104+c40, a104+c41, a104+c42, a104+c43, a104+c44, a104+c45, a104+c46, a104+c47, a104+c48, a104+c49, a104+c50, a104+c51, a104+c52, a104+c53, a104+c54, a104+c55, a104+c56, a104+c57, a104+c58, a104+c59, a104+c60, a104+c61, a104+c62, a104+c63, a104+c64, a104+c65, a104+c66, a104+c67, a104+c68, a104+c69, a104+c70, a104+c71, a104+c72, a104+c73, a104+c74, a104+c75, a104+c76, a104+c77, a104+c78, a104+c79, a104+c80, a104+c81, a104+c82, a104+c83, a104+c84, a104+c85, a104+c86, a104+c87, a104+c88, a104+c89, a104+c90, a104+c91, a104+c92, a104+c93, a104+c94, a104+c95, a104+c96, a104+c97, a104+c98, a104+c99, a104+c100, a104+c101, a104+c102, a104+c103, a104+c104, a104+c105, a104+c106, a104+c107, a104+c108, a104+c109, a104+c110, a104+c111, a104+c112, a104+c113, a104+c114, a104+c115, a104+c116, a104+c117, a104+c118, a104+c119, a104+c120, a104+c121, a104+c122, a104+c123, a104+c124, a104+c125, a104+c126, a104+c127, a104+c128, a104+c129, a104+c130, a104+c131, a104+c132, a104+c133, a104+c134, a104+c135, a104+c136, a104+c137, a104+c138, a104+c139, a104+c140, a104+c141, a104+c142, a104+c143, a104+c144, a104+c145, a104+c146, a104+c147, a104+c148, a104+c149, a104+c150, a104+c151, a104+c152, a104+c153, a104+c154, a104+c155, a104+c156, a104+c157, a104+c158, a104+c159, a104+c160, a104+c161, a104+c162, a104+c163, a104+c164, a104+c165, a104+c166, a104+c167, a104+c168, a104+c169, a104+c170, a104+c171, a104+c172, a104+c173, a104+c174, a104+c175, a104+c176, a104+c177, a104+c178, a104+c179, a104+c180, a104+c181, a104+c182, a104+c183, a104+c184, a104+c185, a104+c186, a104+c187, a104+c188, a104+c189, a104+c190, a104+c191, a104+c192, a104+c193, a104+c194, a104+c195, a104+c196, a104+c197, a104+c198, a104+c199, a104+c200, a104+c201, a104+c202, a104+c203, a104+c204, a104+c205, a104+c206, a104+c207, a104+c208, a104+c209, a104+c210, and a104+c211, and wherein the at least one module (a), the at least one module (b), and the at least one module (c), and the at least one compound (d) are linked to each other in any arrangement and stoichiometry.

Preferably, the conjugate comprises, essentially consists of, consists of or contains:

(a) at least one module (a) that mediates cell targeting and facilitates cellular uptake,
b) at least one module (b) that facilitates transport to the ER,
(c) at least one module (c) that mediates translocation from the ER to the cytosol, and
(d) at least one compound (d),
wherein the at least one module (b) and the at least one module (c) are selected from the group of combinations consisting of b1+c1, b1+c2, b1+c3, b1+c4, b1+c5, b1+c6, b1+c7, b1+c8, b1+c9, b1+c10, b1+c11, b1+c12, b1+c13, b1+c14, b1+c15, b1+c16, b1+c17, b1+c18, b1+c19, b1+c20, b1+c21, b1+c22, b1+c23, b1+c24, b1+c25, b1+c26, b1+c27, b1+c28, b1+c29, b1+c30, b1+c31, b1+c32, b1+c33, b1+c34, b1+c35, b1+c36, b1+c37, b1+c38, b1+c39, b1+c40, b1+c41, b1+c42, b1+c43, b1+c44, b1+c45, b1+c46, b1+c47, b1+c48, b1+c49, b1+c50, b1+c51, b1+c52, b1+c53, b1+c54, b1+c55, b1+c56, b1+c57, b1+c58, b1+c59, b1+c60, b1+c61, b1+c62, b1+c63, b1+c64, b1+c65, b1+c66, b1+c67, b1+c68, b1+c69, b1+c70, b1+c71, b1+c72, b1+c73, b1+c74, b1+c75, b1+c76, b1+c77, b1+c78, b1+c79, b1+c80, b1+c81, b1+c82, b1+c83, b1+c84, b1+c85, b1+c86, b1+c87, b1+c88, b1+c89, b1+c90, b1+c91, b1+c92, b1+c93, b1+c94, b1+c95, b1+c96, b1+c97, b1+c98, b1+c99, b1+c100, b1+c101, b1+c102, b1+c103, b1+c104, b1+c105, b1+c106, b1+c107, b1+c108, b1+c109, b1+c110, b1+c111, b1+c112, b1+c113, b1+c114, b1+c115, b1+c116, b1+c117, b1+c118, b1+c119, b1+c120, b1+c121, b1+c122, b1+c123, b1+c124, b1+c125, b1+c126, b1+c127, b1+c128, b1+c129, b1+c130, b1+c131, b1+c132, b1+c133, b1+c134, b1+c135, b1+c136, b1+c137, b1+c138, b1+c139, b1+c140, b1+c141, b1+c142, b1+c143, b1+c144, b1+c145, b1+c146, b1+c147, b1+c148, b1+c149, b1+c150, b1+c151, b1+c152, b1+c153, b1+c154, b1+c155, b1+c156, b1+c157, b1+c158, b1+c159, b1+c160, b1+c161, b1+c162, b1+c163, b1+c164, b1+c165, b1+c166, b1+c167, b1+c168, b1+c169, b1+c170, b1+c171, b1+c172, b1+c173, b1+c174, b1+c175, b1+c176, b1+c177, b1+c178, b1+c179, b1+c180, b1+c181, b1+c182, b1+c183, b1+c184, b1+c185, b1+c186, b1+c187, b1+c188, b1+c189, b1+c190, b1+c191, b1+c192, b1+c193, b1+c194, b1+c195, b1+c196, b1+c197, b1+c198, b1+c199, b1+c200, b1+c201, b1+c202, b1+c203, b1+c204, b1+c205, b1+c206, b1+c207, b1+c208, b1+c209, b1+c210, b1+c211, b2+c1, b2+c2, b2+c3, b2+c4, b2+c5, b2+c6, b2+c7, b2+c8, b2+c9, b2+c10, b2+c11, b2+c12, b2+c13, b2+c14, b2+c15, b2+c16, b2+c17, b2+c18, b2+c19, b2+c20, b2+c21, b2+c22, b2+c23, b2+c24, b2+c25, b2+c26, b2+c27, b2+c28, b2+c29, b2+c30, b2+c31, b2+c32, b2+c33, b2+c34, b2+c35, b2+c36, b2+c37, b2+c38, b2+c39, b2+c40, b2+c41, b2+c42, b2+c43, b2+c44, b2+c45, b2+c46, b2+c47, b2+c48, b2+c49, b2+c50, b2+c51, b2+c52, b2+c53, b2+c54, b2+c55, b2+c56, b2+c57, b2+c58, b2+c59, b2+c60, b2+c61, b2+c62, b2+c63, b2+c64, b2+c65, b2+c66, b2+c67, b2+c68, b2+c69, b2+c70, b2+c71, b2+c72, b2+c73, b2+c74, b2+c75, b2+c76, b2+c77, b2+c78, b2+c79, b2+c80, b2+c81, b2+c82, b2+c83, b2+c84, b2+c85, b2+c86, b2+c87, b2+c88, b2+c89, b2+c90, b2+c91, b2+c92, b2+c93, b2+c94, b2+c95, b2+c96, b2+c97, b2+c98, b2+c99, b2+c100, b2+c101, b2+c102, b2+c103, b2+c104, b2+c105, b2+c106, b2+c107, b2+c108, b2+c109, b2+c110, b2+c111, b2+c112, b2+c113, b2+c114, b2+c115, b2+c116, b2+c117, b2+c118, b2+c119, b2+c120, b2+c121, b2+c122, b2+c123, b2+c124, b2+c125, b2+c126, b2+c127, b2+c128, b2+c129, b2+c130, b2+c131, b2+c132, b2+c133, b2+c134, b2+c135, b2+c136, b2+c137, b2+c138, b2+c139, b2+c140, b2+c141, b2+c142, b2+c143, b2+c144, b2+c145, b2+c146, b2+c147, b2+c148, b2+c149, b2+c150, b2+c151, b2+c152, b2+c153, b2+c154, b2+c155, b2+c156, b2+c157, b2+c158, b2+c159, b2+c160, b2+c161, b2+c162, b2+c163, b2+c164, b2+c165, b2+c166, b2+c167, b2+c168, b2+c169, b2+c170, b2+c171, b2+c172, b2+c173, b2+c174, b2+c175, b2+c176, b2+c177, b2+c178, b2+c179, b2+c180, b2+c181, b2+c182, b2+c183, b2+c184, b2+c185, b2+c186, b2+c187, b2+c188, b2+c189, b2+c190, b2+c191, b2+c192, b2+c193, b2+c194, b2+c195, b2+c196, b2+c197, b2+c198, b2+c199, b2+c200, b2+c201, b2+c202, b2+c203, b2+c204, b2+c205, b2+c206, b2+c207, b2+c208, b2+c209, b2+c210, b2+c211, b3+c1, b3+c2, b3+c3, b3+c4, b3+c5, b3+c6, b3+c7, b3+c8, b3+c9, b3+c10, b3+c11, b3+c12, b3+c13, b3+c14, b3+c15, b3+c16, b3+c17, b3+c18, b3+c19, b3+c20, b3+c21, b3+c22, b3+c23, b3+c24, b3+c25, b3+c26, b3+c27, b3+c28, b3+c29, b3+c30, b3+c31, b3+c32, b3+c33, b3+c34, b3+c35, b3+c36, b3+c37, b3+c38, b3+c39, b3+c40, b3+c41, b3+c42, b3+c43, b3+c44, b3+c45, b3+c46, b3+c47, b3+c48, b3+c49, b3+c50, b3+c51, b3+c52, b3+c53, b3+c54, b3+c55, b3+c56, b3+c57, b3+c58, b3+c59, b3+c60, b3+c61, b3+c62, b3+c63, b3+c64, b3+c65, b3+c66, b3+c67, b3+c68, b3+c69, b3+c70, b3+c71, b3+c72, b3+c73, b3+c74, b3+c75, b3+c76, b3+c77, b3+c78, b3+c79, b3+c80, b3+c81, b3+c82, b3+c83, b3+c84, b3+c85, b3+c86, b3+c87, b3+c88, b3+c89, b3+c90, b3+c91, b3+c92, b3+c93, b3+c94, b3+c95, b3+c96, b3+c97, b3+c98, b3+c99, b3+c100, b3+c101, b3+c102, b3+c103, b3+c104, b3+c105, b3+c106, b3+c107, b3+c108, b3+c109, b3+c110, b3+c111, b3+c112, b3+c113, b3+c114, b3+c115, b3+c116, b3+c117, b3+c118, b3+c119, b3+c120, b3+c121, b3+c122, b3+c123, b3+c124, b3+c125, b3+c126, b3+c127, b3+c128, b3+c129, b3+c130, b3+c131, b3+c132, b3+c133, b3+c134, b3+c135, b3+c136, b3+c137, b3+c138, b3+c139, b3+c140, b3+c141, b3+c142, b3+c143, b3+c144, b3+c145, b3+c146, b3+c147, b3+c148, b3+c149, b3+c150, b3+c151, b3+c152, b3+c153, b3+c154, b3+c155, b3+c156, b3+c157, b3+c158, b3+c159, b3+c160, b3+c161, b3+c162, b3+c163, b3+c164, b3+c165, b3+c166, b3+c167, b3+c168, b3+c169, b3+c170, b3+c171, b3+c172, b3+c173, b3+c174, b3+c175, b3+c176, b3+c177, b3+c178, b3+c179, b3+c180, b3+c181, b3+c182, b3+c183, b3+c184, b3+c185, b3+c186, b3+c187, b3+c188, b3+c189, b3+c190, b3+c191, b3+c192, b3+c193, b3+c194, b3+c195, b3+c196, b3+c197, b3+c198, b3+c199, b3+c200, b3+c201, b3+c202, b3+c203, b3+c204, b3+c205, b3+c206, b3+c207, b3+c208, b3+c209, b3+c210, b3+c211, b4+c1, b4+c2, b4+c3, b4+c4, b4+c5, b4+c6, b4+c7, b4+c8, b4+c9, b4+c10, b4+c11, b4+c12, b4+c13, b4+c14, b4+c15, b4+c16, b4+c17, b4+c18, b4+c19, b4+c20, b4+c21, b4+c22, b4+c23, b4+c24, b4+c25, b4+c26, b4+c27, b4+c28, b4+c29, b4+c30, b4+c31, b4+c32, b4+c33, b4+c34, b4+c35, b4+c36, b4+c37, b4+c38, b4+c39, b4+c40, b4+c41, b4+c42, b4+c43, b4+c44, b4+c45, b4+c46, b4+c47, b4+c48, b4+c49, b4+c50, b4+c51, b4+c52, b4+c53, b4+c54, b4+c55, b4+c56, b4+c57, b4+c58, b4+c59, b4+c60, b4+c61, b4+c62, b4+c63, b4+c64, b4+c65, b4+c66, b4+c67, b4+c68, b4+c69, b4+c70, b4+c71, b4+c72, b4+c73, b4+c74, b4+c75, b4+c76, b4+c77, b4+c78, b4+c79, b4+c80, b4+c81, b4+c82, b4+c83, b4+c84, b4+c85, b4+c86, b4+c87, b4+c88, b4+c89, b4+c90, b4+c91, b4+c92, b4+c93, b4+c94, b4+c95, b4+c96, b4+c97, b4+c98, b4+c99, b4+c100, b4+c101, b4+c102, b4+c103, b4+c104, b4+c105, b4+c106, b4+c107, b4+c108, b4+c109, b4+c110, b4+c111, b4+c112, b4+c113, b4+c114, b4+c115, b4+c116, b4+c117, b4+c118, b4+c119, b4+c120, b4+c121, b4+c122, b4+c123, b4+c124, b4+c125, b4+c126, b4+c127, b4+c128, b4+c129, b4+c130, b4+c131, b4+c132, b4+c133, b4+c134, b4+c135, b4+c136, b4+c137, b4+c138, b4+c139, b4+c140, b4+c141, b4+c142, b4+c143, b4+c144, b4+c145, b4+c146, b4+c147, b4+c148, b4+c149, b4+c150, b4+c151, b4+c152, b4+c153, b4+c154, b4+c155, b4+c156, b4+c157, b4+c158, b4+c159, b4+c160, b4+c161, b4+c162, b4+c163, b4+c164, b4+c165, b4+c166, b4+c167, b4+c168, b4+c169, b4+c170, b4+c171, b4+c172, b4+c173, b4+c174, b4+c175, b4+c176, b4+c177, b4+c178, b4+c179, b4+c180, b4+c181, b4+c182, b4+c183, b4+c184, b4+c185, b4+c186, b4+c187, b4+c188, b4+c189, b4+c190, b4+c191, b4+c192, b4+c193, b4+c194, b4+c195, b4+c196, b4+c197, b4+c198, b4+c199, b4+c200, b4+c201, b4+c202, b4+c203, b4+c204, b4+c205, b4+c206, b4+c207, b4+c208, b4+c209, b4+c210, b4+c211, b5+c1, b5+c2, b5+c3, b5+c4, b5+c5, b5+c6, b5+c7, b5+c8, b5+c9, b5+c10, b5+c11, b5+c12, b5+c13, b5+c14, b5+c15, b5+c16, b5+c17, b5+c18, b5+c19, b5+c20, b5+c21, b5+c22, b5+c23, b5+c24, b5+c25, b5+c26, b5+c27, b5+c28, b5+c29, b5+c30, b5+c31, b5+c32, b5+c33, b5+c34, b5+c35, b5+c36, b5+c37, b5+c38, b5+c39, b5+c40, b5+c41, b5+c42, b5+c43, b5+c44, b5+c45, b5+c46, b5+c47, b5+c48, b5+c49, b5+c50, b5+c51, b5+c52, b5+c53, b5+c54, b5+c55, b5+c56, b5+c57, b5+c58, b5+c59, b5+c60, b5+c61, b5+c62, b5+c63, b5+c64, b5+c65, b5+c66, b5+c67, b5+c68, b5+c69, b5+c70, b5+c71, b5+c72, b5+c73, b5+c74, b5+c75, b5+c76, b5+c77, b5+c78, b5+c79, b5+c80, b5+c81, b5+c82, b5+c83, b5+c84, b5+c85, b5+c86, b5+c87, b5+c88, b5+c89, b5+c90, b5+c91, b5+c92, b5+c93, b5+c94, b5+c95, b5+c96, b5+c97, b5+c98, b5+c99, b5+c100, b5+c101, b5+c102, b5+c103, b5+c104, b5+c105, b5+c106, b5+c107, b5+c108, b5+c109, b5+c110, b5+c111, b5+c112, b5+c113, b5+c114, b5+c115, b5+c116, b5+c117, b5+c118, b5+c119, b5+c120, b5+c121, b5+c122, b5+c123, b5+c124, b5+c125, b5+c126, b5+c127, b5+c128, b5+c129, b5+c130, b5+c131, b5+c132, b5+c133, b5+c134, b5+c135, b5+c136, b5+c137, b5+c138, b5+c139, b5+c140, b5+c141, b5+c142, b5+c143, b5+c144, b5+c145, b5+c146, b5+c147, b5+c148, b5+c149, b5+c150, b5+c151, b5+c152, b5+c153, b5+c154, b5+c155, b5+c156, b5+c157, b5+c158, b5+c159, b5+c160, b5+c161, b5+c162, b5+c163, b5+c164, b5+c165, b5+c166, b5+c167, b5+c168, b5+c169, b5+c170, b5+c171, b5+c172, b5+c173, b5+c174, b5+c175, b5+c176, b5+c177, b5+c178, b5+c179, b5+c180, b5+c181, b5+c182, b5+c183, b5+c184, b5+c185, b5+c186, b5+c187, b5+c188, b5+c189, b5+c190, b5+c191, b5+c192, b5+c193, b5+c194, b5+c195, b5+c196, b5+c197, b5+c198, b5+c199, b5+c200, b5+c201, b5+c202, b5+c203, b5+c204, b5+c205, b5+c206, b5+c207, b5+c208, b5+c209, b5+c210, b5+c211, b6+c1, b6+c2, b6+c3, b6+c4, b6+c5, b6+c6, b6+c7, b6+c8, b6+c9, b6+c10, b6+c11, b6+c12, b6+c13, b6+c14, b6+c15, b6+c16, b6+c17, b6+c18, b6+c19, b6+c20, b6+c21, b6+c22, b6+c23, b6+c24, b6+c25, b6+c26, b6+c27, b6+c28, b6+c29, b6+c30, b6+c31, b6+c32, b6+c33, b6+c34, b6+c35, b6+c36, b6+c37, b6+c38, b6+c39, b6+c40, b6+c41, b6+c42, b6+c43, b6+c44, b6+c45, b6+c46, b6+c47, b6+c48, b6+c49, b6+c50, b6+c51, b6+c52, b6+c53, b6+c54, b6+c55, b6+c56, b6+c57, b6+c58, b6+c59, b6+c60, b6+c61, b6+c62, b6+c63, b6+c64, b6+c65, b6+c66, b6+c67, b6+c68, b6+c69, b6+c70, b6+c71, b6+c72, b6+c73, b6+c74, b6+c75, b6+c76, b6+c77, b6+c78, b6+c79, b6+c80, b6+c81, b6+c82, b6+c83, b6+c84, b6+c85, b6+c86, b6+c87, b6+c88, b6+c89, b6+c90, b6+c91, b6+c92, b6+c93, b6+c94, b6+c95, b6+c96, b6+c97, b6+c98, b6+c99, b6+c100, b6+c101, b6+c102, b6+c103, b6+c104, b6+c105, b6+c106, b6+c107, b6+c108, b6+c109, b6+c110, b6+c111, b6+c112, b6+c113, b6+c114, b6+c115, b6+c116, b6+c117, b6+c118, b6+c119, b6+c120, b6+c121, b6+c122, b6+c123, b6+c124, b6+c125, b6+c126, b6+c127, b6+c128, b6+c129, b6+c130, b6+c131, b6+c132, b6+c133, b6+c134, b6+c135, b6+c136, b6+c137, b6+c138, b6+c139, b6+c140, b6+c141, b6+c142, b6+c143, b6+c144, b6+c145, b6+c146, b6+c147, b6+c148, b6+c149, b6+c150, b6+c151, b6+c152, b6+c153, b6+c154, b6+c155, b6+c156, b6+c157, b6+c158, b6+c159, b6+c160, b6+c161, b6+c162, b6+c163, b6+c164, b6+c165, b6+c166, b6+c167, b6+c168, b6+c169, b6+c170, b6+c171, b6+c172, b6+c173, b6+c174, b6+c175, b6+c176, b6+c177, b6+c178, b6+c179, b6+c180, b6+c181, b6+c182, b6+c183, b6+c184, b6+c185, b6+c186, b6+c187, b6+c188, b6+c189, b6+c190, b6+c191, b6+c192, b6+c193, b6+c194, b6+c195, b6+c196, b6+c197, b6+c198, b6+c199, b6+c200, b6+c201, b6+c202, b6+c203, b6+c204, b6+c205, b6+c206, b6+c207, b6+c208, b6+c209, b6+c210, b6+c211, b7+c1, b7+c2, b7+c3, b7+c4, b7+c5, b7+c6, b7+c7, b7+c8, b7+c9, b7+c10, b7+c11, b7+c12, b7+c13, b7+c14, b7+c15, b7+c16, b7+c17, b7+c18, b7+c19, b7+c20, b7+c21, b7+c22, b7+c23, b7+c24, b7+c25, b7+c26, b7+c27, b7+c28, b7+c29, b7+c30, b7+c31, b7+c32, b7+c33, b7+c34, b7+c35, b7+c36, b7+c37, b7+c38, b7+c39, b7+c40, b7+c41, b7+c42, b7+c43, b7+c44, b7+c45, b7+c46, b7+c47, b7+c48, b7+c49, b7+c50, b7+c51, b7+c52, b7+c53, b7+c54, b7+c55, b7+c56, b7+c57, b7+c58, b7+c59, b7+c60, b7+c61, b7+c62, b7+c63, b7+c64, b7+c65, b7+c66, b7+c67, b7+c68, b7+c69, b7+c70, b7+c71, b7+c72, b7+c73, b7+c74, b7+c75, b7+c76, b7+c77, b7+c78, b7+c79, b7+c80, b7+c81, b7+c82, b7+c83, b7+c84, b7+c85, b7+c86, b7+c87, b7+c88, b7+c89, b7+c90, b7+c91, b7+c92, b7+c93, b7+c94, b7+c95, b7+c96, b7+c97, b7+c98, b7+c99, b7+c100, b7+c101, b7+c102, b7+c103, b7+c104, b7+c105, b7+c106, b7+c107, b7+c108, b7+c109, b7+c110, b7+c111, b7+c112, b7+c113, b7+c114, b7+c115, b7+c116, b7+c117, b7+c118, b7+c119, b7+c120, b7+c121, b7+c122, b7+c123, b7+c124, b7+c125, b7+c126, b7+c127, b7+c128, b7+c129, b7+c130, b7+c131, b7+c132, b7+c133, b7+c134, b7+c135, b7+c136, b7+c137, b7+c138, b7+c139, b7+c140, b7+c141, b7+c142, b7+c143, b7+c144, b7+c145, b7+c146, b7+c147, b7+c148, b7+c149, b7+c150, b7+c151, b7+c152, b7+c153, b7+c154, b7+c155, b7+c156, b7+c157, b7+c158, b7+c159, b7+c160, b7+c161, b7+c162, b7+c163, b7+c164, b7+c165, b7+c166, b7+c167, b7+c168, b7+c169, b7+c170, b7+c171, b7+c172, b7+c173, b7+c174, b7+c175, b7+c176, b7+c177, b7+c178, b7+c179, b7+c180, b7+c181, b7+c182, b7+c183, b7+c184, b7+c185, b7+c186, b7+c187, b7+c188, b7+c189, b7+c190, b7+c191, b7+c192, b7+c193, b7+c194, b7+c195, b7+c196, b7+c197, b7+c198, b7+c199, b7+c200, b7+c201, b7+c202, b7+c203, b7+c204, b7+c205, b7+c206, b7+c207, b7+c208, b7+c209, b7+c210, b7+c211, b8+c1, b8+c2, b8+c3, b8+c4, b8+c5, b8+c6, b8+c7, b8+c8, b8+c9, b8+c10, b8+c11, b8+c12, b8+c13, b8+c14, b8+c15, b8+c16, b8+c17, b8+c18, b8+c19, b8+c20, b8+c21, b8+c22, b8+c23, b8+c24, b8+c25, b8+c26, b8+c27, b8+c28, b8+c29, b8+c30, b8+c31, b8+c32, b8+c33, b8+c34, b8+c35, b8+c36, b8+c37, b8+c38, b8+c39, b8+c40, b8+c41, b8+c42, b8+c43, b8+c44, b8+c45, b8+c46, b8+c47, b8+c48, b8+c49, b8+c50, b8+c51, b8+c52, b8+c53, b8+c54, b8+c55, b8+c56, b8+c57, b8+c58, b8+c59, b8+c60, b8+c61, b8+c62, b8+c63, b8+c64, b8+c65, b8+c66, b8+c67, b8+c68, b8+c69, b8+c70, b8+c71, b8+c72, b8+c73, b8+c74, b8+c75, b8+c76, b8+c77, b8+c78, b8+c79, b8+c80, b8+c81, b8+c82, b8+c83, b8+c84, b8+c85, b8+c86, b8+c87, b8+c88, b8+c89, b8+c90, b8+c91, b8+c92, b8+c93, b8+c94, b8+c95, b8+c96, b8+c97, b8+c98, b8+c99, b8+c100, b8+c101, b8+c102, b8+c103, b8+c104, b8+c105, b8+c106, b8+c107, b8+c108, b8+c109, b8+c110, b8+c111, b8+c112, b8+c113, b8+c114, b8+c115, b8+c116, b8+c117, b8+c118, b8+c119, b8+c120, b8+c121, b8+c122, b8+c123, b8+c124, b8+c125, b8+c126, b8+c127, b8+c128, b8+c129, b8+c130, b8+c131, b8+c132, b8+c133, b8+c134, b8+c135, b8+c136, b8+c137, b8+c138, b8+c139, b8+c140, b8+c141, b8+c142, b8+c143, b8+c144, b8+c145, b8+c146, b8+c147, b8+c148, b8+c149, b8+c150, b8+c151, b8+c152, b8+c153, b8+c154, b8+c155, b8+c156, b8+c157, b8+c158, b8+c159, b8+c160, b8+c161, b8+c162, b8+c163, b8+c164, b8+c165, b8+c166, b8+c167, b8+c168, b8+c169, b8+c170, b8+c171, b8+c172, b8+c173, b8+c174, b8+c175, b8+c176, b8+c177, b8+c178, b8+c179, b8+c180, b8+c181, b8+c182, b8+c183, b8+c184, b8+c185, b8+c186, b8+c187, b8+c188, b8+c189, b8+c190, b8+c191, b8+c192, b8+c193, b8+c194, b8+c195, b8+c196, b8+c197, b8+c198, b8+c199, b8+c200, b8+c201, b8+c202, b8+c203, b8+c204, b8+c205, b8+c206, b8+c207, b8+c208, b8+c209, b8+c210, b8+c211, b9+c1, b9+c2, b9+c3, b9+c4, b9+c5, b9+c6, b9+c7, b9+c8, b9+c9, b9+c10, b9+c11, b9+c12, b9+c13, b9+c14, b9+c15, b9+c16, b9+c17, b9+c18, b9+c19, b9+c20, b9+c21, b9+c22, b9+c23, b9+c24, b9+c25, b9+c26, b9+c27, b9+c28, b9+c29, b9+c30, b9+c31, b9+c32, b9+c33, b9+c34, b9+c35, b9+c36, b9+c37, b9+c38, b9+c39, b9+c40, b9+c41, b9+c42, b9+c43, b9+c44, b9+c45, b9+c46, b9+c47, b9+c48, b9+c49, b9+c50, b9+c51, b9+c52, b9+c53, b9+c54, b9+c55, b9+c56, b9+c57, b9+c58, b9+c59, b9+c60, b9+c61, b9+c62, b9+c63, b9+c64, b9+c65, b9+c66, b9+c67, b9+c68, b9+c69, b9+c70, b9+c71, b9+c72, b9+c73, b9+c74, b9+c75, b9+c76, b9+c77, b9+c78, b9+c79, b9+c80, b9+c81, b9+c82, b9+c83, b9+c84, b9+c85, b9+c86, b9+c87, b9+c88, b9+c89, b9+c90, b9+c91, b9+c92, b9+c93, b9+c94, b9+c95, b9+c96, b9+c97, b9+c98, b9+c99, b9+c100, b9+c101, b9+c102, b9+c103, b9+c104, b9+c105, b9+c106, b9+c107, b9+c108, b9+c109, b9+c110, b9+c111, b9+c112, b9+c113, b9+c114, b9+c115, b9+c116, b9+c117, b9+c118, b9+c119, b9+c120, b9+c121, b9+c122, b9+c123, b9+c124, b9+c125, b9+c126, b9+c127, b9+c128, b9+c129, b9+c130, b9+c131, b9+c132, b9+c133, b9+c134, b9+c135, b9+c136, b9+c137, b9+c138, b9+c139, b9+c140, b9+c141, b9+c142, b9+c143, b9+c144, b9+c145, b9+c146, b9+c147, b9+c148, b9+c149, b9+c150, b9+c151, b9+c152, b9+c153, b9+c154, b9+c155, b9+c156, b9+c157, b9+c158, b9+c159, b9+c160, b9+c161, b9+c162, b9+c163, b9+c164, b9+c165, b9+c166, b9+c167, b9+c168, b9+c169, b9+c170, b9+c171, b9+c172, b9+c173, b9+c174, b9+c175, b9+c176, b9+c177, b9+c178, b9+c179, b9+c180, b9+c181, b9+c182, b9+c183, b9+c184, b9+c185, b9+c186, b9+c187, b9+c188, b9+c189, b9+c190, b9+c191, b9+c192, b9+c193, b9+c194, b9+c195, b9+c196, b9+c197, b9+c198, b9+c199, b9+c200, b9+c201, b9+c202, b9+c203, b9+c204, b9+c205, b9+c206, b9+c207, b9+c208, b9+c209, b9+c210, b9+c211, b10+c1, b10+c2, b10+c3, b10+c4, b10+c5, b10+c6, b10+c7, b10+c8, b10+c9, b10+c10, b10+c11, b10+c12, b10+c13, b10+c14, b10+c15, b10+c16, b10+c17, b10+c18, b10+c19, b10+c20, b10+c21, b10+c22, b10+c23, b10+c24, b10+c25, b10+c26, b10+c27, b10+c28, b10+c29, b10+c30, b10+c31, b10+c32, b10+c33, b10+c34, b10+c35, b10+c36, b10+c37, b10+c38, b10+c39, b10+c40, b10+c41, b10+c42, b10+c43, b10+c44, b10+c45, b10+c46, b10+c47, b10+c48, b10+c49, b10+c50, b10+c51, b10+c52, b10+c53, b10+c54, b10+c55, b10+c56, b10+c57, b10+c58, b10+c59, b10+c60, b10+c61, b10+c62, b10+c63, b10+c64, b10+c65, b10+c66, b10+c67, b10+c68, b10+c69, b10+c70, b10+c71, b10+c72, b10+c73, b10+c74, b10+c75, b10+c76, b10+c77, b10+c78, b10+c79, b10+c80, b10+c81, b10+c82, b10+c83, b10+c84, b10+c85, b10+c86, b10+c87, b10+c88, b10+c89, b10+c90, b10+c91, b10+c92, b10+c93, b10+c94, b10+c95, b10+c96, b10+c97, b10+c98, b10+c99, b10+c100, b10+c101, b10+c102, b10+c103, b10+c104, b10+c105, b10+c106, b10+c107, b10+c108, b10+c109, b10+c110, b10+c111, b10+c112, b10+c113, b10+c114, b10+c115, b10+c116, b10+c117, b10+c118, b10+c119, b10+c120, b10+c121, b10+c122, b10+c123, b10+c124, b10+c125, b10+c126, b10+c127, b10+c128, b10+c129, b10+c130, b10+c131, b10+c132, b10+c133, b10+c134, b10+c135, b10+c136, b10+c137, b10+c138, b10+c139, b10+c140, b10+c141, b10+c142, b10+c143, b10+c144, b10+c145, b10+c146, b10+c147, b10+c148, b10+c149, b10+c150, b10+c151, b10+c152, b10+c153, b10+c154, b10+c155, b10+c156, b10+c157, b10+c158, b10+c159, b10+c160, b10+c161, b10+c162, b10+c163, b10+c164, b10+c165, b10+c166, b10+c167, b10+c168, b10+c169, b10+c170, b10+c171, b10+c172, b10+c173, b10+c174, b10+c175, b10+c176, b10+c177, b10+c178, b10+c179, b10+c180, b10+c181, b10+c182, b10+c183, b10+c184, b10+c185, b10+c186, b10+c187, b10+c188, b10+c189, b10+c190, b10+c191, b10+c192, b10+c193, b10+c194, b10+c195, b10+c196, b10+c197, b10+c198, b10+c199, b10+c200, b10+c201, b10+c202, b10+c203, b10+c204, b10+c205, b10+c206, b10+c207, b10+c208, b10+c209, b10+c210, b10+c211, b11+c1, b11+c2, b11+c3, b11+c4, b11+c5, b11+c6, b11+c7, b11+c8, b11+c9, b11+c10, b11+c11, b11+c12, b11+c13, b11+c14, b11+c15, b11+c16, b11+c17, b11+c18, b11+c19, b11+c20, b11+c21, b11+c22, b11+c23, b11+c24, b11+c25, b11+c26, b11+c27, b11+c28, b11+c29, b11+c30, b11+c31, b11+c32, b11+c33, b11+c34, b11+c35, b11+c36, b11+c37, b11+c38, b11+c39, b11+c40, b11+c41, b11+c42, b11+c43, b11+c44, b11+c45, b11+c46, b11+c47, b11+c48, b11+c49, b11+c50, b11+c51, b11+c52, b11+c53, b11+c54, b11+c55, b11+c56, b11+c57, b11+c58, b11+c59, b11+c60, b11+c61, b11+c62, b11+c63, b11+c64, b11+c65, b11+c66, b11+c67, b11+c68, b11+c69, b11+c70, b11+c71, b11+c72, b11+c73, b11+c74, b11+c75, b11+c76, b11+c77, b11+c78, b11+c79, b11+c80, b11+c81, b11+c82, b11+c83, b11+c84, b11+c85, b11+c86, b11+c87, b11+c88, b11+c89, b11+c90, b11+c91, b11+c92, b11+c93, b11+c94, b11+c95, b11+c96, b11+c97, b11+c98, b11+c99, b11+c100, b11+c101, b11+c102, b11+c103, b11+c104, b11+c105, b11+c106, b11+c107, b11+c108, b11+c109, b11+c110, b11+c111, b11+c112, b11+c113, b11+c114, b11+c115, b11+c116, b11+c117, b11+c118, b11+c119, b11+c120, b11+c121, b11+c122, b11+c123, b11+c124, b11+c125, b11+c126, b11+c127, b11+c128, b11+c129, b11+c130, b11+c131, b11+c132, b11+c133, b11+c134, b11+c135, b11+c136, b11+c137, b11+c138, b11+c139, b11+c140, b11+c141, b11+c142, b11+c143, b11+c144, b11+c145, b11+c146, b11+c147, b11+c148, b11+c149, b11+c150, b11+c151, b11+c152, b11+c153, b11+c154, b11+c155, b11+c156, b11+c157, b11+c158, b11+c159, b11+c160, b11+c161, b11+c162, b11+c163, b11+c164, b11+c165, b11+c166, b11+c167, b11+c168, b11+c169, b11+c170, b11+c171, b11+c172, b11+c173, b11+c174, b11+c175, b11+c176, b11+c177, b11+c178, b11+c179, b11+c180, b11+c181, b11+c182, b11+c183, b11+c184, b11+c185, b11+c186, b11+c187, b11+c188, b11+c189, b11+c190, b11+c191, b11+c192, b11+c193, b11+c194, b11+c195, b11+c196, b11+c197, b11+c198, b11+c199, b11+c200, b11+c201, b11+c202, b11+c203, b11+c204, b11+c205, b11+c206, b11+c207, b11+c208, b11+c209, b11+c210, b11+c211, b12+c1, b12+c2, b12+c3, b12+c4, b12+c5, b12+c6, b12+c7, b12+c8, b12+c9, b12+c10, b12+c11, b12+c12, b12+c13, b12+c14, b12+c15, b12+c16, b12+c17, b12+c18, b12+c19, b12+c20, b12+c21, b12+c22, b12+c23, b12+c24, b12+c25, b12+c26, b12+c27, b12+c28, b12+c29, b12+c30, b12+c31, b12+c32, b12+c33, b12+c34, b12+c35, b12+c36, b12+c37, b12+c38, b12+c39, b12+c40, b12+c41, b12+c42, b12+c43, b12+c44, b12+c45, b12+c46, b12+c47, b12+c48, b12+c49, b12+c50, b12+c51, b12+c52, b12+c53, b12+c54, b12+c55, b12+c56, b12+c57, b12+c58, b12+c59, b12+c60, b12+c61, b12+c62, b12+c63, b12+c64, b12+c65, b12+c66, b12+c67, b12+c68, b12+c69, b12+c70, b12+c71, b12+c72, b12+c73, b12+c74, b12+c75, b12+c76, b12+c77, b12+c78, b12+c79, b12+c80, b12+c81, b12+c82, b12+c83, b12+c84, b12+c85, b12+c86, b12+c87, b12+c88, b12+c89, b12+c90, b12+c91, b12+c92, b12+c93, b12+c94, b12+c95, b12+c96, b12+c97, b12+c98, b12+c99, b12+c100, b12+c101, b12+c102, b12+c103, b12+c104, b12+c105, b12+c106, b12+c107, b12+c108, b12+c109, b12+c110, b12+c111, b12+c112, b12+c113, b12+c114, b12+c115, b12+c116, b12+c117, b12+c118, b12+c119, b12+c120, b12+c121, b12+c122, b12+c123, b12+c124, b12+c125, b12+c126, b12+c127, b12+c128, b12+c129, b12+c130, b12+c131, b12+c132, b12+c133, b12+c134, b12+c135, b12+c136, b12+c137, b12+c138, b12+c139, b12+c140, b12+c141, b12+c142, b12+c143, b12+c144, b12+c145, b12+c146, b12+c147, b12+c148, b12+c149, b12+c150, b12+c151, b12+c152, b12+c153, b12+c154, b12+c155, b12+c156, b12+c157, b12+c158, b12+c159, b12+c160, b12+c161, b12+c162, b12+c163, b12+c164, b12+c165, b12+c166, b12+c167, b12+c168, b12+c169, b12+c170, b12+c171, b12+c172, b12+c173, b12+c174, b12+c175, b12+c176, b12+c177, b12+c178, b12+c179, b12+c180, b12+c181, b12+c182, b12+c183, b12+c184, b12+c185, b12+c186, b12+c187, b12+c188, b12+c189, b12+c190, b12+c191, b12+c192, b12+c193, b12+c194, b12+c195, b12+c196, b12+c197, b12+c198, b12+c199, b12+c200, b12+c201, b12+c202, b12+c203, b12+c204, b12+c205, b12+c206, b12+c207, b12+c208, b12+c209, b12+c210, b12+c211, b13+c1, b13+c2, b13+c3, b13+c4, b13+c5, b13+c6, b13+c7, b13+c8, b13+c9, b13+c10, b13+c11, b13+c12, b13+c13, b13+c14, b13+c15, b13+c16, b13+c17, b13+c18, b13+c19, b13+c20, b13+c21, b13+c22, b13+c23, b13+c24, b13+c25, b13+c26, b13+c27, b13+c28, b13+c29, b13+c30, b13+c31, b13+c32, b13+c33, b13+c34, b13+c35, b13+c36, b13+c37, b13+c38, b13+c39, b13+c40, b13+c41, b13+c42, b13+c43, b13+c44, b13+c45, b13+c46, b13+c47, b13+c48, b13+c49, b13+c50, b13+c51, b13+c52, b13+c53, b13+c54, b13+c55, b13+c56, b13+c57, b13+c58, b13+c59, b13+c60, b13+c61, b13+c62, b13+c63, b13+c64, b13+c65, b13+c66, b13+c67, b13+c68, b13+c69, b13+c70, b13+c71, b13+c72, b13+c73, b13+c74, b13+c75, b13+c76, b13+c77, b13+c78, b13+c79, b13+c80, b13+c81, b13+c82, b13+c83, b13+c84, b13+c85, b13+c86, b13+c87, b13+c88, b13+c89, b13+c90, b13+c91, b13+c92, b13+c93, b13+c94, b13+c95, b13+c96, b13+c97, b13+c98, b13+c99, b13+c100, b13+c101, b13+c102, b13+c103, b13+c104, b13+c105, b13+c106, b13+c107, b13+c108, b13+c109, b13+c110, b13+c111, b13+c112, b13+c113, b13+c114, b13+c115, b13+c116, b13+c117, b13+c118, b13+c119, b13+c120, b13+c121, b13+c122, b13+c123, b13+c124, b13+c125, b13+c126, b13+c127, b13+c128, b13+c129, b13+c130, b13+c131, b13+c132, b13+c133, b13+c134, b13+c135, b13+c136, b13+c137, b13+c138, b13+c139, b13+c140, b13+c141, b13+c142, b13+c143, b13+c144, b13+c145, b13+c146, b13+c147, b13+c148, b13+c149, b13+c150, b13+c151, b13+c152, b13+c153, b13+c154, b13+c155, b13+c156, b13+c157, b13+c158, b13+c159, b13+c160, b13+c161, b13+c162, b13+c163, b13+c164, b13+c165, b13+c166, b13+c167, b13+c168, b13+c169, b13+c170, b13+c171, b13+c172, b13+c173, b13+c174, b13+c175, b13+c176, b13+c177, b13+c178, b13+c179, b13+c180, b13+c181, b13+c182, b13+c183, b13+c184, b13+c185, b13+c186, b13+c187, b13+c188, b13+c189, b13+c190, b13+c191, b13+c192, b13+c193, b13+c194, b13+c195, b13+c196, b13+c197, b13+c198, b13+c199, b13+c200, b13+c201, b13+c202, b13+c203, b13+c204, b13+c205, b13+c206, b13+c207, b13+c208, b13+c209, b13+c210, b13+c211, b14+c1, b14+c2, b14+c3, b14+c4, b14+c5, b14+c6, b14+c7, b14+c8, b14+c9, b14+c10, b14+c11, b14+c12, b14+c13, b14+c14, b14+c15, b14+c16, b14+c17, b14+c18, b14+c19, b14+c20, b14+c21, b14+c22, b14+c23, b14+c24, b14+c25, b14+c26, b14+c27, b14+c28, b14+c29, b14+c30, b14+c31, b14+c32, b14+c33, b14+c34, b14+c35, b14+c36, b14+c37, b14+c38, b14+c39, b14+c40, b14+c41, b14+c42, b14+c43, b14+c44, b14+c45, b14+c46, b14+c47, b14+c48, b14+c49, b14+c50, b14+c51, b14+c52, b14+c53, b14+c54, b14+c55, b14+c56, b14+c57, b14+c58, b14+c59, b14+c60, b14+c61, b14+c62, b14+c63, b14+c64, b14+c65, b14+c66, b14+c67, b14+c68, b14+c69, b14+c70, b14+c71, b14+c72, b14+c73, b14+c74, b14+c75, b14+c76, b14+c77, b14+c78, b14+c79, b14+c80, b14+c81, b14+c82, b14+c83, b14+c84, b14+c85, b14+c86, b14+c87, b14+c88, b14+c89, b14+c90, b14+c91, b14+c92, b14+c93, b14+c94, b14+c95, b14+c96, b14+c97, b14+c98, b14+c99, b14+c100, b14+c101, b14+c102, b14+c103, b14+c104, b14+c105, b14+c106, b14+c107, b14+c108, b14+c109, b14+c110, b14+c111, b14+c112, b14+c113, b14+c114, b14+c115, b14+c116, b14+c117, b14+c118, b14+c119, b14+c120, b14+c121, b14+c122, b14+c123, b14+c124, b14+c125, b14+c126, b14+c127, b14+c128, b14+c129, b14+c130, b14+c131, b14+c132, b14+c133, b14+c134, b14+c135, b14+c136, b14+c137, b14+c138, b14+c139, b14+c140, b14+c141, b14+c142, b14+c143, b14+c144, b14+c145, b14+c146, b14+c147, b14+c148, b14+c149, b14+c150, b14+c151, b14+c152, b14+c153, b14+c154, b14+c155, b14+c156, b14+c157, b14+c158, b14+c159, b14+c160, b14+c161, b14+c162, b14+c163, b14+c164, b14+c165, b14+c166, b14+c167, b14+c168, b14+c169, b14+c170, b14+c171, b14+c172, b14+c173, b14+c174, b14+c175, b14+c176, b14+c177, b14+c178, b14+c179, b14+c180, b14+c181, b14+c182, b14+c183, b14+c184, b14+c185, b14+c186, b14+c187, b14+c188, b14+c189, b14+c190, b14+c191, b14+c192, b14+c193, b14+c194, b14+c195, b14+c196, b14+c197, b14+c198, b14+c199, b14+c200, b14+c201, b14+c202, b14+c203, b14+c204, b14+c205, b14+c206, b14+c207, b14+c208, b14+c209, b14+c210, b14+c211, b15+c1, b15+c2, b15+c3, b15+c4, b15+c5, b15+c6, b15+c7, b15+c8, b15+c9, b15+c10, b15+c11, b15+c12, b15+c13, b15+c14, b15+c15, b15+c16, b15+c17, b15+c18, b15+c19, b15+c20, b15+c21, b15+c22, b15+c23, b15+c24, b15+c25, b15+c26, b15+c27, b15+c28, b15+c29, b15+c30, b15+c31, b15+c32, b15+c33, b15+c34, b15+c35, b15+c36, b15+c37, b15+c38, b15+c39, b15+c40, b15+c41, b15+c42, b15+c43, b15+c44, b15+c45, b15+c46, b15+c47, b15+c48, b15+c49, b15+c50, b15+c51, b15+c52, b15+c53, b15+c54, b15+c55, b15+c56, b15+c57, b15+c58, b15+c59, b15+c60, b15+c61, b15+c62, b15+c63, b15+c64, b15+c65, b15+c66, b15+c67, b15+c68, b15+c69, b15+c70, b15+c71, b15+c72, b15+c73, b15+c74, b15+c75, b15+c76, b15+c77, b15+c78, b15+c79, b15+c80, b15+c81, b15+c82, b15+c83, b15+c84, b15+c85, b15+c86, b15+c87, b15+c88, b15+c89, b15+c90, b15+c91, b15+c92, b15+c93, b15+c94, b15+c95, b15+c96, b15+c97, b15+c98, b15+c99, b15+c100, b15+c101, b15+c102, b15+c103, b15+c104, b15+c105, b15+c106, b15+c107, b15+c108, b15+c109, b15+c110, b15+c111, b15+c112, b15+c113, b15+c114, b15+c115, b15+c116, b15+c117, b15+c118, b15+c119, b15+c120, b15+c121, b15+c122, b15+c123, b15+c124, b15+c125, b15+c126, b15+c127, b15+c128, b15+c129, b15+c130, b15+c131, b15+c132, b15+c133, b15+c134, b15+c135, b15+c136, b15+c137, b15+c138, b15+c139, b15+c140, b15+c141, b15+c142, b15+c143, b15+c144, b15+c145, b15+c146, b15+c147, b15+c148, b15+c149, b15+c150, b15+c151, b15+c152, b15+c153, b15+c154, b15+c155, b15+c156, b15+c157, b15+c158, b15+c159, b15+c160, b15+c161, b15+c162, b15+c163, b15+c164, b15+c165, b15+c166, b15+c167, b15+c168, b15+c169, b15+c170, b15+c171, b15+c172, b15+c173, b15+c174, b15+c175, b15+c176, b15+c177, b15+c178, b15+c179, b15+c180, b15+c181, b15+c182, b15+c183, b15+c184, b15+c185, b15+c186, b15+c187, b15+c188, b15+c189, b15+c190, b15+c191, b15+c192, b15+c193, b15+c194, b15+c195, b15+c196, b15+c197, b15+c198, b15+c199, b15+c200, b15+c201, b15+c202, b15+c203, b15+c204, b15+c205, b15+c206, b15+c207, b15+c208, b15+c209, b15+c210, b15+c211, b16+c1, b16+c2, b16+c3, b16+c4, b16+c5, b16+c6, b16+c7, b16+c8, b16+c9, b16+c10, b16+c11, b16+c12, b16+c13, b16+c14, b16+c15, b16+c16, b16+c17, b16+c18, b16+c19, b16+c20, b16+c21, b16+c22, b16+c23, b16+c24, b16+c25, b16+c26, b16+c27, b16+c28, b16+c29, b16+c30, b16+c31, b16+c32, b16+c33, b16+c34, b16+c35, b16+c36, b16+c37, b16+c38, b16+c39, b16+c40, b16+c41, b16+c42, b16+c43, b16+c44, b16+c45, b16+c46, b16+c47, b16+c48, b16+c49, b16+c50, b16+c51, b16+c52, b16+c53, b16+c54, b16+c55, b16+c56, b16+c57, b16+c58, b16+c59, b16+c60, b16+c61, b16+c62, b16+c63, b16+c64, b16+c65, b16+c66, b16+c67, b16+c68, b16+c69, b16+c70, b16+c71, b16+c72, b16+c73, b16+c74, b16+c75, b16+c76, b16+c77, b16+c78, b16+c79, b16+c80, b16+c81, b16+c82, b16+c83, b16+c84, b16+c85, b16+c86, b16+c87, b16+c88, b16+c89, b16+c90, b16+c91, b16+c92, b16+c93, b16+c94, b16+c95, b16+c96, b16+c97, b16+c98, b16+c99, b16+c100, b16+c101, b16+c102, b16+c103, b16+c104, b16+c105, b16+c106, b16+c107, b16+c108, b16+c109, b16+c110, b16+c111, b16+c112, b16+c113, b16+c114, b16+c115, b16+c116, b16+c117, b16+c118, b16+c119, b16+c120, b16+c121, b16+c122, b16+c123, b16+c124, b16+c125, b16+c126, b16+c127, b16+c128, b16+c129, b16+c130, b16+c131, b16+c132, b16+c133, b16+c134, b16+c135, b16+c136, b16+c137, b16+c138, b16+c139, b16+c140, b16+c141, b16+c142, b16+c143, b16+c144, b16+c145, b16+c146, b16+c147, b16+c148, b16+c149, b16+c150, b16+c151, b16+c152, b16+c153, b16+c154, b16+c155, b16+c156, b16+c157, b16+c158, b16+c159, b16+c160, b16+c161, b16+c162, b16+c163, b16+c164, b16+c165, b16+c166, b16+c167, b16+c168, b16+c169, b16+c170, b16+c171, b16+c172, b16+c173, b16+c174, b16+c175, b16+c176, b16+c177, b16+c178, b16+c179, b16+c180, b16+c181, b16+c182, b16+c183, b16+c184, b16+c185, b16+c186, b16+c187, b16+c188, b16+c189, b16+c190, b16+c191, b16+c192, b16+c193, b16+c194, b16+c195, b16+c196, b16+c197, b16+c198, b16+c199, b16+c200, b16+c201, b16+c202, b16+c203, b16+c204, b16+c205, b16+c206, b16+c207, b16+c208, b16+c209, b16+c210, b16+c211, b17+c1, b17+c2, b17+c3, b17+c4, b17+c5, b17+c6, b17+c7, b17+c8, b17+c9, b17+c10, b17+c11, b17+c12, b17+c13, b17+c14, b17+c15, b17+c16, b17+c17, b17+c18, b17+c19, b17+c20, b17+c21, b17+c22, b17+c23, b17+c24, b17+c25, b17+c26, b17+c27, b17+c28, b17+c29, b17+c30, b17+c31, b17+c32, b17+c33, b17+c34, b17+c35, b17+c36, b17+c37, b17+c38, b17+c39, b17+c40, b17+c41, b17+c42, b17+c43, b17+c44, b17+c45, b17+c46, b17+c47, b17+c48, b17+c49, b17+c50, b17+c51, b17+c52, b17+c53, b17+c54, b17+c55, b17+c56, b17+c57, b17+c58, b17+c59, b17+c60, b17+c61, b17+c62, b17+c63, b17+c64, b17+c65, b17+c66, b17+c67, b17+c68, b17+c69, b17+c70, b17+c71, b17+c72, b17+c73, b17+c74, b17+c75, b17+c76, b17+c77, b17+c78, b17+c79, b17+c80, b17+c81, b17+c82, b17+c83, b17+c84, b17+c85, b17+c86, b17+c87, b17+c88, b17+c89, b17+c90, b17+c91, b17+c92, b17+c93, b17+c94, b17+c95, b17+c96, b17+c97, b17+c98, b17+c99, b17+c100, b17+c101, b17+c102, b17+c103, b17+c104, b17+c105, b17+c106, b17+c107, b17+c108, b17+c109, b17+c110, b17+c111, b17+c112, b17+c113, b17+c114, b17+c115, b17+c116, b17+c117, b17+c118, b17+c119, b17+c120, b17+c121, b17+c122, b17+c123, b17+c124, b17+c125, b17+c126, b17+c127, b17+c128, b17+c129, b17+c130, b17+c131, b17+c132, b17+c133, b17+c134, b17+c135, b17+c136, b17+c137, b17+c138, b17+c139, b17+c140, b17+c141, b17+c142, b17+c143, b17+c144, b17+c145, b17+c146, b17+c147, b17+c148, b17+c149, b17+c150, b17+c151, b17+c152, b17+c153, b17+c154, b17+c155, b17+c156, b17+c157, b17+c158, b17+c159, b17+c160, b17+c161, b17+c162, b17+c163, b17+c164, b17+c165, b17+c166, b17+c167, b17+c168, b17+c169, b17+c170, b17+c171, b17+c172, b17+c173, b17+c174, b17+c175, b17+c176, b17+c177, b17+c178, b17+c179, b17+c180, b17+c181, b17+c182, b17+c183, b17+c184, b17+c185, b17+c186, b17+c187, b17+c188, b17+c189, b17+c190, b17+c191, b17+c192, b17+c193, b17+c194, b17+c195, b17+c196, b17+c197, b17+c198, b17+c199, b17+c200, b17+c201, b17+c202, b17+c203, b17+c204, b17+c205, b17+c206, b17+c207, b17+c208, b17+c209, b17+c210, b17+c211, b18+c1, b18+c2, b18+c3, b18+c4, b18+c5, b18+c6, b18+c7, b18+c8, b18+c9, b18+c10, b18+c11, b18+c12, b18+c13, b18+c14, b18+c15, b18+c16, b18+c17, b18+c18, b18+c19, b18+c20, b18+c21, b18+c22, b18+c23, b18+c24, b18+c25, b18+c26, b18+c27, b18+c28, b18+c29, b18+c30, b18+c31, b18+c32, b18+c33, b18+c34, b18+c35, b18+c36, b18+c37, b18+c38, b18+c39, b18+c40, b18+c41, b18+c42, b18+c43, b18+c44, b18+c45, b18+c46, b18+c47, b18+c48, b18+c49, b18+c50, b18+c51, b18+c52, b18+c53, b18+c54, b18+c55, b18+c56, b18+c57, b18+c58, b18+c59, b18+c60, b18+c61, b18+c62, b18+c63, b18+c64, b18+c65, b18+c66, b18+c67, b18+c68, b18+c69, b18+c70, b18+c71, b18+c72, b18+c73, b18+c74, b18+c75, b18+c76, b18+c77, b18+c78, b18+c79, b18+c80, b18+c81, b18+c82, b18+c83, b18+c84, b18+c85, b18+c86, b18+c87, b18+c88, b18+c89, b18+c90, b18+c91, b18+c92, b18+c93, b18+c94, b18+c95, b18+c96, b18+c97, b18+c98, b18+c99, b18+c100, b18+c101, b18+c102, b18+c103, b18+c104, b18+c105, b18+c106, b18+c107, b18+c108, b18+c109, b18+c110, b18+c111, b18+c112, b18+c113, b18+c114, b18+c115, b18+c116, b18+c117, b18+c118, b18+c119, b18+c120, b18+c121, b18+c122, b18+c123, b18+c124, b18+c125, b18+c126, b18+c127, b18+c128, b18+c129, b18+c130, b18+c131, b18+c132, b18+c133, b18+c134, b18+c135, b18+c136, b18+c137, b18+c138, b18+c139, b18+c140, b18+c141, b18+c142, b18+c143, b18+c144, b18+c145, b18+c146, b18+c147, b18+c148, b18+c149, b18+c150, b18+c151, b18+c152, b18+c153, b18+c154, b18+c155, b18+c156, b18+c157, b18+c158, b18+c159, b18+c160, b18+c161, b18+c162, b18+c163, b18+c164, b18+c165, b18+c166, b18+c167, b18+c168, b18+c169, b18+c170, b18+c171, b18+c172, b18+c173, b18+c174, b18+c175, b18+c176, b18+c177, b18+c178, b18+c179, b18+c180, b18+c181, b18+c182, b18+c183, b18+c184, b18+c185, b18+c186, b18+c187, b18+c188, b18+c189, b18+c190, b18+c191, b18+c192, b18+c193, b18+c194, b18+c195, b18+c196, b18+c197, b18+c198, b18+c199, b18+c200, b18+c201, b18+c202, b18+c203, b18+c204, b18+c205, b18+c206, b18+c207, b18+c208, b18+c209, b18+c210, b18+c211, b19+c1, b19+c2, b19+c3, b19+c4, b19+c5, b19+c6, b19+c7, b19+c8, b19+c9, b19+c10, b19+c11, b19+c12, b19+c13, b19+c14, b19+c15, b19+c16, b19+c17, b19+c18, b19+c19, b19+c20, b19+c21, b19+c22, b19+c23, b19+c24, b19+c25, b19+c26, b19+c27, b19+c28, b19+c29, b19+c30, b19+c31, b19+c32, b19+c33, b19+c34, b19+c35, b19+c36, b19+c37, b19+c38, b19+c39, b19+c40, b19+c41, b19+c42, b19+c43, b19+c44, b19+c45, b19+c46, b19+c47, b19+c48, b19+c49, b19+c50, b19+c51, b19+c52, b19+c53, b19+c54, b19+c55, b19+c56, b19+c57, b19+c58, b19+c59, b19+c60, b19+c61, b19+c62, b19+c63, b19+c64, b19+c65, b19+c66, b19+c67, b19+c68, b19+c69, b19+c70, b19+c71, b19+c72, b19+c73, b19+c74, b19+c75, b19+c76, b19+c77, b19+c78, b19+c79, b19+c80, b19+c81, b19+c82, b19+c83, b19+c84, b19+c85, b19+c86, b19+c87, b19+c88, b19+c89, b19+c90, b19+c91, b19+c92, b19+c93, b19+c94, b19+c95, b19+c96, b19+c97, b19+c98, b19+c99, b19+c100, b19+c101, b19+c102, b19+c103, b19+c104, b19+c105, b19+c106, b19+c107, b19+c108, b19+c109, b19+c110, b19+c111, b19+c112, b19+c113, b19+c114, b19+c115, b19+c116, b19+c117, b19+c118, b19+c119, b19+c120, b19+c121, b19+c122, b19+c123, b19+c124, b19+c125, b19+c126, b19+c127, b19+c128, b19+c129, b19+c130, b19+c131, b19+c132, b19+c133, b19+c134, b19+c135, b19+c136, b19+c137, b19+c138, b19+c139, b19+c140, b19+c141, b19+c142, b19+c143, b19+c144, b19+c145, b19+c146, b19+c147, b19+c148, b19+c149, b19+c150, b19+c151, b19+c152, b19+c153, b19+c154, b19+c155, b19+c156, b19+c157, b19+c158, b19+c159, b19+c160, b19+c161, b19+c162, b19+c163, b19+c164, b19+c165, b19+c166, b19+c167, b19+c168, b19+c169, b19+c170, b19+c171, b19+c172, b19+c173, b19+c174, b19+c175, b19+c176, b19+c177, b19+c178, b19+c179, b19+c180, b19+c181, b19+c182, b19+c183, b19+c184, b19+c185, b19+c186, b19+c187, b19+c188, b19+c189, b19+c190, b19+c191, b19+c192, b19+c193, b19+c194, b19+c195, b19+c196, b19+c197, b19+c198, b19+c199, b19+c200, b19+c201, b19+c202, b19+c203, b19+c204, b19+c205, b19+c206, b19+c207, b19+c208, b19+c209, b19+c210, b19+c211, b20+c1, b20+c2, b20+c3, b20+c4, b20+c5, b20+c6, b20+c7, b20+c8, b20+c9, b20+c10, b20+c11, b20+c12, b20+c13, b20+c14, b20+c15, b20+c16, b20+c17, b20+c18, b20+c19, b20+c20, b20+c21, b20+c22, b20+c23, b20+c24, b20+c25, b20+c26, b20+c27, b20+c28, b20+c29, b20+c30, b20+c31, b20+c32, b20+c33, b20+c34, b20+c35, b20+c36, b20+c37, b20+c38, b20+c39, b20+c40, b20+c41, b20+c42, b20+c43, b20+c44, b20+c45, b20+c46, b20+c47, b20+c48, b20+c49, b20+c50, b20+c51, b20+c52, b20+c53, b20+c54, b20+c55, b20+c56, b20+c57, b20+c58, b20+c59, b20+c60, b20+c61, b20+c62, b20+c63, b20+c64, b20+c65, b20+c66, b20+c67, b20+c68, b20+c69, b20+c70, b20+c71, b20+c72, b20+c73, b20+c74, b20+c75, b20+c76, b20+c77, b20+c78, b20+c79, b20+c80, b20+c81, b20+c82, b20+c83, b20+c84, b20+c85, b20+c86, b20+c87, b20+c88, b20+c89, b20+c90, b20+c91, b20+c92, b20+c93, b20+c94, b20+c95, b20+c96, b20+c97, b20+c98, b20+c99, b20+c100, b20+c101, b20+c102, b20+c103, b20+c104, b20+c105, b20+c106, b20+c107, b20+c108, b20+c109, b20+c110, b20+c111, b20+c112, b20+c113, b20+c114, b20+c115, b20+c116, b20+c117, b20+c118, b20+c119, b20+c120, b20+c121, b20+c122, b20+c123, b20+c124, b20+c125, b20+c126, b20+c127, b20+c128, b20+c129, b20+c130, b20+c131, b20+c132, b20+c133, b20+c134, b20+c135, b20+c136, b20+c137, b20+c138, b20+c139, b20+c140, b20+c141, b20+c142, b20+c143, b20+c144, b20+c145, b20+c146, b20+c147, b20+c148, b20+c149, b20+c150, b20+c151, b20+c152, b20+c153, b20+c154, b20+c155, b20+c156, b20+c157, b20+c158, b20+c159, b20+c160, b20+c161, b20+c162, b20+c163, b20+c164, b20+c165, b20+c166, b20+c167, b20+c168, b20+c169, b20+c170, b20+c171, b20+c172, b20+c173, b20+c174, b20+c175, b20+c176, b20+c177, b20+c178, b20+c179, b20+c180, b20+c181, b20+c182, b20+c183, b20+c184, b20+c185, b20+c186, b20+c187, b20+c188, b20+c189, b20+c190, b20+c191, b20+c192, b20+c193, b20+c194, b20+c195, b20+c196, b20+c197, b20+c198, b20+c199, b20+c200, b20+c201, b20+c202, b20+c203, b20+c204, b20+c205, b20+c206, b20+c207, b20+c208, b20+c209, b20+c210, b20+c211, b21+c1, b21+c2, b21+c3, b21+c4, b21+c5, b21+c6, b21+c7, b21+c8, b21+c9, b21+c10, b21+c11, b21+c12, b21+c13, b21+c14, b21+c15, b21+c16, b21+c17, b21+c18, b21+c19, b21+c20, b21+c21, b21+c22, b21+c23, b21+c24, b21+c25, b21+c26, b21+c27, b21+c28, b21+c29, b21+c30, b21+c31, b21+c32, b21+c33, b21+c34, b21+c35, b21+c36, b21+c37, b21+c38, b21+c39, b21+c40, b21+c41, b21+c42, b21+c43, b21+c44, b21+c45, b21+c46, b21+c47, b21+c48, b21+c49, b21+c50, b21+c51, b21+c52, b21+c53, b21+c54, b21+c55, b21+c56, b21+c57, b21+c58, b21+c59, b21+c60, b21+c61, b21+c62, b21+c63, b21+c64, b21+c65, b21+c66, b21+c67, b21+c68, b21+c69, b21+c70, b21+c71, b21+c72, b21+c73, b21+c74, b21+c75, b21+c76, b21+c77, b21+c78, b21+c79, b21+c80, b21+c81, b21+c82, b21+c83, b21+c84, b21+c85, b21+c86, b21+c87, b21+c88, b21+c89, b21+c90, b21+c91, b21+c92, b21+c93, b21+c94, b21+c95, b21+c96, b21+c97, b21+c98, b21+c99, b21+c100, b21+c101, b21+c102, b21+c103, b21+c104, b21+c105, b21+c106, b21+c107, b21+c108, b21+c109, b21+c110, b21+c111, b21+c112, b21+c113, b21+c114, b21+c115, b21+c116, b21+c117, b21+c118, b21+c119, b21+c120, b21+c121, b21+c122, b21+c123, b21+c124, b21+c125, b21+c126, b21+c127, b21+c128, b21+c129, b21+c130, b21+c131, b21+c132, b21+c133, b21+c134, b21+c135, b21+c136, b21+c137, b21+c138, b21+c139, b21+c140, b21+c141, b21+c142, b21+c143, b21+c144, b21+c145, b21+c146, b21+c147, b21+c148, b21+c149, b21+c150, b21+c151, b21+c152, b21+c153, b21+c154, b21+c155, b21+c156, b21+c157, b21+c158, b21+c159, b21+c160, b21+c161, b21+c162, b21+c163, b21+c164, b21+c165, b21+c166, b21+c167, b21+c168, b21+c169, b21+c170, b21+c171, b21+c172, b21+c173, b21+c174, b21+c175, b21+c176, b21+c177, b21+c178, b21+c179, b21+c180, b21+c181, b21+c182, b21+c183, b21+c184, b21+c185, b21+c186, b21+c187, b21+c188, b21+c189, b21+c190, b21+c191, b21+c192, b21+c193, b21+c194, b21+c195, b21+c196, b21+c197, b21+c198, b21+c199, b21+c200, b21+c201, b21+c202, b21+c203, b21+c204, b21+c205, b21+c206, b21+c207, b21+c208, b21+c209, b21+c210, b21+c211, b22+c1, b22+c2, b22+c3, b22+c4, b22+c5, b22+c6, b22+c7, b22+c8, b22+c9, b22+c10, b22+c11, b22+c12, b22+c13, b22+c14, b22+c15, b22+c16, b22+c17, b22+c18, b22+c19, b22+c20, b22+c21, b22+c22, b22+c23, b22+c24, b22+c25, b22+c26, b22+c27, b22+c28, b22+c29, b22+c30, b22+c31, b22+c32, b22+c33, b22+c34, b22+c35, b22+c36, b22+c37, b22+c38, b22+c39, b22+c40, b22+c41, b22+c42, b22+c43, b22+c44, b22+c45, b22+c46, b22+c47, b22+c48, b22+c49, b22+c50, b22+c51, b22+c52, b22+c53, b22+c54, b22+c55, b22+c56, b22+c57, b22+c58, b22+c59, b22+c60, b22+c61, b22+c62, b22+c63, b22+c64, b22+c65, b22+c66, b22+c67, b22+c68, b22+c69, b22+c70, b22+c71, b22+c72, b22+c73, b22+c74, b22+c75, b22+c76, b22+c77, b22+c78, b22+c79, b22+c80, b22+c81, b22+c82, b22+c83, b22+c84, b22+c85, b22+c86, b22+c87, b22+c88, b22+c89, b22+c90, b22+c91, b22+c92, b22+c93, b22+c94, b22+c95, b22+c96, b22+c97, b22+c98, b22+c99, b22+c100, b22+c101, b22+c102, b22+c103, b22+c104, b22+c105, b22+c106, b22+c107, b22+c108, b22+c109, b22+c110, b22+c111, b22+c112, b22+c113, b22+c114, b22+c115, b22+c116, b22+c117, b22+c118, b22+c119, b22+c120, b22+c121, b22+c122, b22+c123, b22+c124, b22+c125, b22+c126, b22+c127, b22+c128, b22+c129, b22+c130, b22+c131, b22+c132, b22+c133, b22+c134, b22+c135, b22+c136, b22+c137, b22+c138, b22+c139, b22+c140, b22+c141, b22+c142, b22+c143, b22+c144, b22+c145, b22+c146, b22+c147, b22+c148, b22+c149, b22+c150, b22+c151, b22+c152, b22+c153, b22+c154, b22+c155, b22+c156, b22+c157, b22+c158, b22+c159, b22+c160, b22+c161, b22+c162, b22+c163, b22+c164, b22+c165, b22+c166, b22+c167, b22+c168, b22+c169, b22+c170, b22+c171, b22+c172, b22+c173, b22+c174, b22+c175, b22+c176, b22+c177, b22+c178, b22+c179, b22+c180, b22+c181, b22+c182, b22+c183, b22+c184, b22+c185, b22+c186, b22+c187, b22+c188, b22+c189, b22+c190, b22+c191, b22+c192, b22+c193, b22+c194, b22+c195, b22+c196, b22+c197, b22+c198, b22+c199, b22+c200, b22+c201, b22+c202, b22+c203, b22+c204, b22+c205, b22+c206, b22+c207, b22+c208, b22+c209, b22+c210, b22+c211, b23+c1, b23+c2, b23+c3, b23+c4, b23+c5, b23+c6, b23+c7, b23+c8, b23+c9, b23+c10, b23+c11, b23+c12, b23+c13, b23+c14, b23+c15, b23+c16, b23+c17, b23+c18, b23+c19, b23+c20, b23+c21, b23+c22, b23+c23, b23+c24, b23+c25, b23+c26, b23+c27, b23+c28, b23+c29, b23+c30, b23+c31, b23+c32, b23+c33, b23+c34, b23+c35, b23+c36, b23+c37, b23+c38, b23+c39, b23+c40, b23+c41, b23+c42, b23+c43, b23+c44, b23+c45, b23+c46, b23+c47, b23+c48, b23+c49, b23+c50, b23+c51, b23+c52, b23+c53, b23+c54, b23+c55, b23+c56, b23+c57, b23+c58, b23+c59, b23+c60, b23+c61, b23+c62, b23+c63, b23+c64, b23+c65, b23+c66, b23+c67, b23+c68, b23+c69, b23+c70, b23+c71, b23+c72, b23+c73, b23+c74, b23+c75, b23+c76, b23+c77, b23+c78, b23+c79, b23+c80, b23+c81, b23+c82, b23+c83, b23+c84, b23+c85, b23+c86, b23+c87, b23+c88, b23+c89, b23+c90, b23+c91, b23+c92, b23+c93, b23+c94, b23+c95, b23+c96, b23+c97, b23+c98, b23+c99, b23+c100, b23+c101, b23+c102, b23+c103, b23+c104, b23+c105, b23+c106, b23+c107, b23+c108, b23+c109, b23+c110, b23+c111, b23+c112, b23+c113, b23+c114, b23+c115, b23+c116, b23+c117, b23+c118, b23+c119, b23+c120, b23+c121, b23+c122, b23+c123, b23+c124, b23+c125, b23+c126, b23+c127, b23+c128, b23+c129, b23+c130, b23+c131, b23+c132, b23+c133, b23+c134, b23+c135, b23+c136, b23+c137, b23+c138, b23+c139, b23+c140, b23+c141, b23+c142, b23+c143, b23+c144, b23+c145, b23+c146, b23+c147, b23+c148, b23+c149, b23+c150, b23+c151, b23+c152, b23+c153, b23+c154, b23+c155, b23+c156, b23+c157, b23+c158, b23+c159, b23+c160, b23+c161, b23+c162, b23+c163, b23+c164, b23+c165, b23+c166, b23+c167, b23+c168, b23+c169, b23+c170, b23+c171, b23+c172, b23+c173, b23+c174, b23+c175, b23+c176, b23+c177, b23+c178, b23+c179, b23+c180, b23+c181, b23+c182, b23+c183, b23+c184, b23+c185, b23+c186, b23+c187, b23+c188, b23+c189, b23+c190, b23+c191, b23+c192, b23+c193, b23+c194, b23+c195, b23+c196, b23+c197, b23+c198, b23+c199, b23+c200, b23+c201, b23+c202, b23+c203, b23+c204, b23+c205, b23+c206, b23+c207, b23+c208, b23+c209, b23+c210, b23+c211, b24+c1, b24+c2, b24+c3, b24+c4, b24+c5, b24+c6, b24+c7, b24+c8, b24+c9, b24+c10, b24+c11, b24+c12, b24+c13, b24+c14, b24+c15, b24+c16, b24+c17, b24+c18, b24+c19, b24+c20, b24+c21, b24+c22, b24+c23, b24+c24, b24+c25, b24+c26, b24+c27, b24+c28, b24+c29, b24+c30, b24+c31, b24+c32, b24+c33, b24+c34, b24+c35, b24+c36, b24+c37, b24+c38, b24+c39, b24+c40, b24+c41, b24+c42, b24+c43, b24+c44, b24+c45, b24+c46, b24+c47, b24+c48, b24+c49, b24+c50, b24+c51, b24+c52, b24+c53, b24+c54, b24+c55, b24+c56, b24+c57, b24+c58, b24+c59, b24+c60, b24+c61, b24+c62, b24+c63, b24+c64, b24+c65, b24+c66, b24+c67, b24+c68, b24+c69, b24+c70, b24+c71, b24+c72, b24+c73, b24+c74, b24+c75, b24+c76, b24+c77, b24+c78, b24+c79, b24+c80, b24+c81, b24+c82, b24+c83, b24+c84, b24+c85, b24+c86, b24+c87, b24+c88, b24+c89, b24+c90, b24+c91, b24+c92, b24+c93, b24+c94, b24+c95, b24+c96, b24+c97, b24+c98, b24+c99, b24+c100, b24+c101, b24+c102, b24+c103, b24+c104, b24+c105, b24+c106, b24+c107, b24+c108, b24+c109, b24+c110, b24+c111, b24+c112, b24+c113, b24+c114, b24+c115, b24+c116, b24+c117, b24+c118, b24+c119, b24+c120, b24+c121, b24+c122, b24+c123, b24+c124, b24+c125, b24+c126, b24+c127, b24+c128, b24+c129, b24+c130, b24+c131, b24+c132, b24+c133, b24+c134, b24+c135, b24+c136, b24+c137, b24+c138, b24+c139, b24+c140, b24+c141, b24+c142, b24+c143, b24+c144, b24+c145, b24+c146, b24+c147, b24+c148, b24+c149, b24+c150, b24+c151, b24+c152, b24+c153, b24+c154, b24+c155, b24+c156, b24+c157, b24+c158, b24+c159, b24+c160, b24+c161, b24+c162, b24+c163, b24+c164, b24+c165, b24+c166, b24+c167, b24+c168, b24+c169, b24+c170, b24+c171, b24+c172, b24+c173, b24+c174, b24+c175, b24+c176, b24+c177, b24+c178, b24+c179, b24+c180, b24+c181, b24+c182, b24+c183, b24+c184, b24+c185, b24+c186, b24+c187, b24+c188, b24+c189, b24+c190, b24+c191, b24+c192, b24+c193, b24+c194, b24+c195, b24+c196, b24+c197, b24+c198, b24+c199, b24+c200, b24+c201, b24+c202, b24+c203, b24+c204, b24+c205, b24+c206, b24+c207, b24+c208, b24+c209, b24+c210, b24+c211, b25+c1, b25+c2, b25+c3, b25+c4, b25+c5, b25+c6, b25+c7, b25+c8, b25+c9, b25+c10, b25+c11, b25+c12, b25+c13, b25+c14, b25+c15, b25+c16, b25+c17, b25+c18, b25+c19, b25+c20, b25+c21, b25+c22, b25+c23, b25+c24, b25+c25, b25+c26, b25+c27, b25+c28, b25+c29, b25+c30, b25+c31, b25+c32, b25+c33, b25+c34, b25+c35, b25+c36, b25+c37, b25+c38, b25+c39, b25+c40, b25+c41, b25+c42, b25+c43, b25+c44, b25+c45, b25+c46, b25+c47, b25+c48, b25+c49, b25+c50, b25+c51, b25+c52, b25+c53, b25+c54, b25+c55, b25+c56, b25+c57, b25+c58, b25+c59, b25+c60, b25+c61, b25+c62, b25+c63, b25+c64, b25+c65, b25+c66, b25+c67, b25+c68, b25+c69, b25+c70, b25+c71, b25+c72, b25+c73, b25+c74, b25+c75, b25+c76, b25+c77, b25+c78, b25+c79, b25+c80, b25+c81, b25+c82, b25+c83, b25+c84, b25+c85, b25+c86, b25+c87, b25+c88, b25+c89, b25+c90, b25+c91, b25+c92, b25+c93, b25+c94, b25+c95, b25+c96, b25+c97, b25+c98, b25+c99, b25+c100, b25+c101, b25+c102, b25+c103, b25+c104, b25+c105, b25+c106, b25+c107, b25+c108, b25+c109, b25+c110, b25+c111, b25+c112, b25+c113, b25+c114, b25+c115, b25+c116, b25+c117, b25+c118, b25+c119, b25+c120, b25+c121, b25+c122, b25+c123, b25+c124, b25+c125, b25+c126, b25+c127, b25+c128, b25+c129, b25+c130, b25+c131, b25+c132, b25+c133, b25+c134, b25+c135, b25+c136, b25+c137, b25+c138, b25+c139, b25+c140, b25+c141, b25+c142, b25+c143, b25+c144, b25+c145, b25+c146, b25+c147, b25+c148, b25+c149, b25+c150, b25+c151, b25+c152, b25+c153, b25+c154, b25+c155, b25+c156, b25+c157, b25+c158, b25+c159, b25+c160, b25+c161, b25+c162, b25+c163, b25+c164, b25+c165, b25+c166, b25+c167, b25+c168, b25+c169, b25+c170, b25+c171, b25+c172, b25+c173, b25+c174, b25+c175, b25+c176, b25+c177, b25+c178, b25+c179, b25+c180, b25+c181, b25+c182, b25+c183, b25+c184, b25+c185, b25+c186, b25+c187, b25+c188, b25+c189, b25+c190, b25+c191, b25+c192, b25+c193, b25+c194, b25+c195, b25+c196, b25+c197, b25+c198, b25+c199, b25+c200, b25+c201, b25+c202, b25+c203, b25+c204, b25+c205, b25+c206, b25+c207, b25+c208, b25+c209, b25+c210, and b25+c211, and wherein the at least one module (a), the at least one module (b), and the at least one module (c), and the at least one compound (d) are linked to each other in any arrangement and stoichiometry.

Preferably, the conjugate comprises, essentially consists of, consists of or contains:

(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
wherein the at least one module (a) and the at least one module (b) are combined in a combination as indicated by a numerical from K1 to K1750, K4919 to K5768, and wherein the combination of the at least one module (a) and the at least one module (b) is combined with the at least one module (c) according to the following scheme:
KX, in each case combined with at least one module c1; KX, in each case combined with at least one module c2; KX, in each case combined with at least one module c3; KX, in each case combined with at least one module c4; KX, in each case combined with at least one module c5; KX, in each case combined with at least one module c6; KX, in each case combined with at least one module c7; KX, in each case combined with at least one module c8; KX, in each case combined with at least one module c9; KX, in each case combined with at least one module c10; KX, in each case combined with at least one module e11; KX, in each case combined with at least one module c12; KX, in each case combined with at least one module c13; KX, in each case combined with at least one module c14; KX, in each case combined with at least one module c15; KX, in each case combined with at least one module c16; KX, in each case combined with at least one module c17; KX, in each case combined with at least one module c18; KX, in each case combined with at least one module c19; KX, in each case combined with at least one module c20; KX, in each case combined with at least one module c21; KX, in each case combined with at least one module c22; KX, in each case combined with at least one module c23; KX, in each case combined with at least one module c24; KX, in each case combined with at least one module c25; KX, in each case combined with at least one module c26; KX, in each case combined with at least one module c27; KX, in each case combined with at least one module c28; KX, in each case combined with at least one module c29; KX, in each case combined with at least one module c30; KX, in each case combined with at least one module c31; KX, in each case combined with at least one module c32; KX, in each case combined with at least one module c33; KX, in each case combined with at least one module c34; KX, in each case combined with at least one module c35; KX, in each case combined with at least one module c36; KX, in each case combined with at least one module c37; KX, in each case combined with at least one module c38; KX, in each case combined with at least one module c39; KX, in each case combined with at least one module c40; KX, in each case combined with at least one module c41; KX, in each case combined with at least one module c42; KX, in each case combined with at least one module c43; KX, in each case combined with at least one module c44; KX, in each case combined with at least one module c45; KX, in each case combined with at least one module c46; KX, in each case combined with at least one module c47; KX, in each case combined with at least one module c48; KX, in each case combined with at least one module c49; KX, in each case combined with at least one module c50; KX, in each case combined with at least one module c51; KX, in each case combined with at least one module c52; KX, in each case combined with at least one module c53; KX, in each case combined with at least one module c54; KX, in each case combined with at least one module c55; KX, in each case combined with at least one module c56; KX, in each case combined with at least one module c57; KX, in each case combined with at least one module c58; KX, in each case combined with at least one module c59; KX, in each case combined with at least one module c60; KX, in each case combined with at least one module c61; KX, in each case combined with at least one module c62; KX, in each case combined with at least one module c63; KX, in each case combined with at least one module c64; KX, in each case combined with at least one module c65; KX, in each case combined with at least one module c66; KX, in each case combined with at least one module c67; KX, in each case combined with at least one module c68; KX, in each case combined with at least one module c69; KX, in each case combined with at least one module c70; KX, in each case combined with at least one module c71; KX, in each case combined with at least one module c72; KX, in each case combined with at least one module c73; KX, in each case combined with at least one module c74; KX, in each case combined with at least one module c75; KX, in each case combined with at least one module c76; KX, in each case combined with at least one module c77; KX, in each case combined with at least one module c78; KX, in each case combined with at least one module c79; KX, in each case combined with at least one module c80; KX, in each case combined with at least one module c81; KX, in each case combined with at least one module c82; KX, in each case combined with at least one module c83; KX, in each case combined with at least one module c84; KX, in each case combined with at least one module c85; KX, in each case combined with at least one module c86; KX, in each case combined with at least one module c87; KX, in each case combined with at least one module c88; KX, in each case combined with at least one module c89; KX, in each case combined with at least one module c90; KX in each case combined with at least one module c91; KX, in each case combined with at least one module c92; KX, in each case combined with at least one module c93; KX, in each case combined with at least one module c94; KX, in each case combined with at least one module c95; KX, in each case combined with at least one module c96; KX, in each case combined with at least one module c97; KX, in each case combined with at least one module c98; KX, in each case combined with at least one module c99; KX, in each case combined with at least one module c100; KX, in each case combined with at least one module c101; KX, in each case combined with at least one module c102; KX, in each case combined with at least one module c103; KX, in each case combined with at least one module c104; KX, in each case combined with at least one module c105; KX, in each case combined with at least one module c106; KX, in each case combined with at least one module c107; KX, in each case combined with at least one module c108; KX, in each case combined with at least one module c109; KX, in each case combined with at least one module c110; KX, in each case combined with at least one module c111; KX, in each case combined with at least one module c112; KX, in each case combined with at least one module c113; KX, in each case combined with at least one module c114; KX, in each case combined with at least one module c115; KX, in each case combined with at least one module c116; KX, in each case combined with at least one module c117; KX, in each case combined with at least one module c118; KX, in each case combined with at least one module c119; KX, in each case combined with at least one module c120; KX, in each case combined with at least one module c121; KX, in each case combined with at least one module c122; KX, in each case combined with at least one module c123; KX, in each case combined with at least one module c124; KX, in each case combined with at least one module c125; KX, in each case combined with at least one module c126; KX, in each case combined with at least one module c127; KX, in each case combined with at least one module c128; KX, in each case combined with at least one module c129; KX, in each case combined with at least one module c130; KX, in each case combined with at least one module c131; KX, in each case combined with at least one module c132; KX, in each case combined with at least one module c133; KX, in each case combined with at least one module c134; KX, in each case combined with at least one module c135; KX, in each case combined with at least one module c136; KX, in each case combined with at least one module c137; KX, in each case combined with at least one module c138; KX, in each case combined with at least one module c139; KX, in each case combined with at least one module c140; KX, in each case combined with at least one module c141; KX, in each case combined with at least one module c142; KX, in each case combined with at least one module c143; KX, in each case combined with at least one module c144; KX, in each case combined with at least one module c145; KX, in each case combined with at least one module c146; KX, in each case combined with at least one module c147; KX, in each case combined with at least one module c148; KX, in each case combined with at least one module c149; KX, in each case combined with at least one module c150; KX, in each case combined with at least one module c151; KX, in each case combined with at least one module c152; KX, in each case combined with at least one module c153; KX, in each case combined with at least one module c154; KX, in each case combined with at least one module c155; KX, in each case combined with at least one module c156; KX, in each case combined with at least one module c157; KX, in each case combined with at least one module c158; KX, in each case combined with at least one module c159; KX, in each case combined with at least one module c160; KX, in each case combined with at least one module c161; KX, in each case combined with at least one module c162; KX, in each case combined with at least one module c163; KX, in each case combined with at least one module c164; KX, in each case combined with at least one module c165; KX, in each case combined with at least one module c166; KX, in each case combined with at least one module c167; KX, in each case combined with at least one module c168; KX, in each case combined with at least one module c169; KX, in each case combined with at least one module c170; KX, in each case combined with at least one module c171; KX, in each case combined with at least one module c172; KX, in each case combined with at least one module c173; KX, in each case combined with at least one module c174; KX, in each case combined with at least one module c175; KX, in each case combined with at least one module c176; KX, in each case combined with at least one module c177; KX, in each case combined with at least one module c178; KX, in each case combined with at least one module c179; KX, in each case combined with at least one module c180; KX, in each case combined with at least one module c181; KX, in each case combined with at least one module c182; KX, in each case combined with at least one module c183; KX, in each case combined with at least one module c184; KX, in each case combined with at least one module c185; KX, in each case combined with at least one module c186; KX, in each case combined with at least one module c187; KX, in each case combined with at least one module c188; KX, in each case combined with at least one module c189; KX, in each case combined with at least one module c190; KX in each case combined with at least one module c191; KX, in each case combined with at least one module c192; KX, in each case combined with at least one module c193; KX, in each case combined with at least one module c194; KX, in each case combined with at least one module c195; KX, in each case combined with at least one module c196; KX, in each case combined with at least one module c197; KX, in each case combined with at least one module c198; KX, in each case combined with at least one module c199; KX, in each case combined with at least one module c200; KX, in each case combined with at least one module c201; KX, in each case combined with at least one module c202; KX, in each case combined with at least one module c203; KX, in each case combined with at least one module c204; KX, in each case combined with at least one module c205; KX, in each case combined with at least one module c206; KX, in each case combined with at least one module c207; KX, in each case combined with at least one module c208; KX, in each case combined with at least one module c209; KX, in each case combined with at least one module c210; and KX in each case combined with at least one module c211;
wherein X has the following meaning: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1245, 1246, 1247, 1248, 1249, 1250, 1251, 1252, 1253, 1254, 1255, 1256, 1257, 1258, 1259, 1260, 1261, 1262, 1263, 1264, 1265, 1266, 1267, 1268, 1269, 1270, 1271, 1272, 1273, 1274, 1275, 1276, 1277, 1278, 1279, 1280, 1281, 1282, 1283, 1284, 1285, 1286, 1287, 1288, 1289, 1290, 1291, 1292, 1293, 1294, 1295, 1296, 1297, 1298, 1299, 1300, 1301, 1302, 1303, 1304, 1305, 1306, 1307, 1308, 1309, 1310, 1311, 1312, 1313, 1314, 1315, 1316, 1317, 1318, 1319, 1320, 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, 1349, 1350, 1351, 1352, 1353, 1354, 1355, 1356, 1357, 1358, 1359, 1360, 1361, 1362, 1363, 1364, 1365, 1366, 1367, 1368, 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, 1394, 1395, 1396, 1397, 1398, 1399, 1400, 1401, 1402, 1403, 1404, 1405, 1406, 1407, 1408, 1409, 1410, 1411, 1412, 1413, 1414, 1415, 1416, 1417, 1418, 1419, 1420, 1421, 1422, 1423, 1424, 1425, 1426, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, 1503, 1504, 1505, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1604, 1605, 1606, 1607, 1608, 1609, 1610, 1611, 1612, 1613, 1614, 1615, 1616, 1617, 1618, 1619, 1620, 1621, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1649, 1650, 1651, 1652, 1653, 1654, 1655, 1656, 1657, 1658, 1659, 1660, 1661, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, 1732, 1733, 1734, 1735, 1736, 1737, 1738, 1739, 1740, 1741, 1742, 1743, 1744, 1745, 1746, 1747, 1748, 1749, 1750, 4919, 4920, 4921, 4922, 4923, 4924, 4925, 4926, 4927, 4928, 4929, 4930, 4931, 4932, 4933, 4934, 4935, 4936, 4937, 4938, 4939, 4940, 4941, 4942, 4943, 4944, 4945, 4946, 4947, 4948, 4949, 4950, 4951, 4952, 4953, 4954, 4955, 4956, 4957, 4958, 4959, 4960, 4961, 4962, 4963, 4964, 4965, 4966, 4967, 4968, 4969, 4970, 4971, 4972, 4973, 4974, 4975, 4976, 4977, 4978, 4979, 4980, 4981, 4982, 4983, 4984, 4985, 4986, 4987, 4988, 4989, 4990, 4991, 4992, 4993, 4994, 4995, 4996, 4997, 4998, 4999, 5000, 5001, 5002, 5003, 5004, 5005, 5006, 5007, 5008, 5009, 5010, 5011, 5012, 5013, 5014, 5015, 5016, 5017, 5018, 5019, 5020, 5021, 5022, 5023, 5024, 5025, 5026, 5027, 5028, 5029, 5030, 5031, 5032, 5033, 5034, 5035, 5036, 5037, 5038, 5039, 5040, 5041, 5042, 5043, 5044, 5045, 5046, 5047, 5048, 5049, 5050, 5051, 5052, 5053, 5054, 5055, 5056, 5057, 5058, 5059, 5060, 5061, 5062, 5063, 5064, 5065, 5066, 5067, 5068, 5069, 5070, 5071, 5072, 5073, 5074, 5075, 5076, 5077, 5078, 5079, 5080, 5081, 5082, 5083, 5084, 5085, 5086, 5087, 5088, 5089, 5090, 5091, 5092, 5093, 5094, 5095, 5096, 5097, 5098, 5099, 5100, 5101, 5102, 5103, 5104, 5105, 5106, 5107, 5108, 5109, 5110, 5111, 5112, 5113, 5114, 5115, 5116, 5117, 5118, 5119, 5120, 5121, 5122, 5123, 5124, 5125, 5126, 5127, 5128, 5129, 5130, 5131, 5132, 5133, 5134, 5135, 5136, 5137, 5138, 5139, 5140, 5141, 5142, 5143, 5144, 5145, 5146, 5147, 5148, 5149, 5150, 5151, 5152, 5153, 5154, 5155, 5156, 5157, 5158, 5159, 5160, 5161, 5162, 5163, 5164, 5165, 5166, 5167, 5168, 5169, 5170, 5171, 5172, 5173, 5174, 5175, 5176, 5177, 5178, 5179, 5180, 5181, 5182, 5183, 5184, 5185, 5186, 5187, 5188, 5189, 5190, 5191, 5192, 5193, 5194, 5195, 5196, 5197, 5198, 5199, 5200, 5201, 5202, 5203, 5204, 5205, 5206, 5207, 5208, 5209, 5210, 5211, 5212, 5213, 5214, 5215, 5216, 5217, 5218, 5219, 5220, 5221, 5222, 5223, 5224, 5225, 5226, 5227, 5228, 5229, 5230, 5231, 5232, 5233, 5234, 5235, 5236, 5237, 5238, 5239, 5240, 5241, 5242, 5243, 5244, 5245, 5246, 5247, 5248, 5249, 5250, 5251, 5252, 5253, 5254, 5255, 5256, 5257, 5258, 5259, 5260, 5261, 5262, 5263, 5264, 5265, 5266, 5267, 5268, 5269, 5270, 5271, 5272, 5273, 5274, 5275, 5276, 5277, 5278, 5279, 5280, 5281, 5282, 5283, 5284, 5285, 5286, 5287, 5288, 5289, 5290, 5291, 5292, 5293, 5294, 5295, 5296, 5297, 5298, 5299, 5300, 5301, 5302, 5303, 5304, 5305, 5306, 5307, 5308, 5309, 5310, 5311, 5312, 5313, 5314, 5315, 5316, 5317, 5318, 5319, 5320, 5321, 5322, 5323, 5324, 5325, 5326, 5327, 5328, 5329, 5330, 5331, 5332, 5333, 5334, 5335, 5336, 5337, 5338, 5339, 5340, 5341, 5342, 5343, 5344, 5345, 5346, 5347, 5348, 5349, 5350, 5351, 5352, 5353, 5354, 5355, 5356, 5357, 5358, 5359, 5360, 5361, 5362, 5363, 5364, 5365, 5366, 5367, 5368, 5369, 5370, 5371, 5372, 5373, 5374, 5375, 5376, 5377, 5378, 5379, 5380, 5381, 5382, 5383, 5384, 5385, 5386, 5387, 5388, 5389, 5390, 5391, 5392, 5393, 5394, 5395, 5396, 5397, 5398, 5399, 5400, 5401, 5402, 5403, 5404, 5405, 5406, 5407, 5408, 5409, 5410, 5411, 5412, 5413, 5414, 5415, 5416, 5417, 5418, 5419, 5420, 5421, 5422, 5423, 5424, 5425, 5426, 5427, 5428, 5429, 5430, 5431, 5432, 5433, 5434, 5435, 5436, 5437, 5438, 5439, 5440, 5441, 5442, 5443, 5444, 5445, 5446, 5447, 5448, 5449, 5450, 5451, 5452, 5453, 5454, 5455, 5456, 5457, 5458, 5459, 5460, 5461, 5462, 5463, 5464, 5465, 5466, 5467, 5468, 5469, 5470, 5471, 5472, 5473, 5474, 5475, 5476, 5477, 5478, 5479, 5480, 5481, 5482, 5483, 5484, 5485, 5486, 5487, 5488, 5489, 5490, 5491, 5492, 5493, 5494, 5495, 5496, 5497, 5498, 5499, 5500, 5501, 5502, 5503, 5504, 5505, 5506, 5507, 5508, 5509, 5510, 5511, 5512, 5513, 5514, 5515, 5516, 5517, 5518, 5519, 5520, 5521, 5522, 5523, 5524, 5525, 5526, 5527, 5528, 5529, 5530, 5531, 5532, 5533, 5534, 5535, 5536, 5537, 5538, 5539, 5540, 5541, 5542, 5543, 5544, 5545, 5546, 5547, 5548, 5549, 5550, 5551, 5552, 5553, 5554, 5555, 5556, 5557, 5558, 5559, 5560, 5561, 5562, 5563, 5564, 5565, 5566, 5567, 5568, 5569, 5570, 5571, 5572, 5573, 5574, 5575, 5576, 5577, 5578, 5579, 5580, 5581, 5582, 5583, 5584, 5585, 5586, 5587, 5588, 5589, 5590, 5591, 5592, 5593, 5594, 5595, 5596, 5597, 5598, 5599, 5600, 5601, 5602, 5603, 5604, 5605, 5606, 5607, 5608, 5609, 5610, 5611, 5612, 5613, 5614, 5615, 5616, 5617, 5618, 5619, 5620, 5621, 5622, 5623, 5624, 5625, 5626, 5627, 5628, 5629, 5630, 5631, 5632, 5633, 5634, 5635, 5636, 5637, 5638, 5639, 5640, 5641, 5642, 5643, 5644, 5645, 5646, 5647, 5648, 5649, 5650, 5651, 5652, 5653, 5654, 5655, 5656, 5657, 5658, 5659, 5660, 5661, 5662, 5663, 5664, 5665, 5666, 5667, 5668, 5669, 5670, 5671, 5672, 5673, 5674, 5675, 5676, 5677, 5678, 5679, 5680, 5681, 5682, 5683, 5684, 5685, 5686, 5687, 5688, 5689, 5690, 5691, 5692, 5693, 5694, 5695, 5696, 5697, 5698, 5699, 5700, 5701, 5702, 5703, 5704, 5705, 5706, 5707, 5708, 5709, 5710, 5711, 5712, 5713, 5714, 5715, 5716, 5717, 5718, 5719, 5720, 5721, 5722, 5723, 5724, 5725, 5726, 5727, 5728, 5729, 5730, 5731, 5732, 5733, 5734, 5735, 5736, 5737, 5738, 5739, 5740, 5741, 5742, 5743, 5744, 5745, 5746, 5747, 5748, 5749, 5750, 5751, 5752, 5753, 5754, 5755, 5756, 5757, 5758, 5759, 5760, 5761, 5762, 5763, 5764, 5765, 5766, 5767, or 5768, and wherein the at least one module (a), the at least one module (b), the at least one module (c), and the at least one compound (d) are linked to each other in any arrangement and stoichiometry.

Preferably, the conjugate comprises, essentially consists of, consists of or contains:

(a) at least one module (a) that mediates cell targeting and facilitates cellular uptake,
b) at least one module (b) that facilitates transport to the ER,
(c) at least one module (c) that mediates translocation from the ER to the cytosol, and
(d) at least one compound (d),
wherein the at least one module (a) and at least one module (b) are combined in a combination as indicated by a numerical from K1 to K1750, K4919 to K5768, and wherein the combination of the at least one module (a) and the at least one module (b) is combined with at least one module (c) as indicated by a numerical from c1 to c211 and at least one compound (d) according to the following scheme:
KXY, in each case combined with at least one compound d1; KXY, in each case combined with at least one compound d2; KXY, in each case combined with at least one compound d3; KXY, in each case combined with at least one compound d4; KXY, in each case combined with at least one compound d5; KXY, in each case combined with at least one compound d6; KXY, in each case combined with at least one compound d7; KXY, in each case combined with at least one compound d8; KXY, in each case combined with at least one compound d9; KXY, in each case combined with at least one compound d10; KXY, in each case combined with at least one compound d11; KXY, in each case combined with at least one compound d12; KXY, in each case combined with at least one compound d13; KXY, in each case combined with at least one compound d14; KXY, in each case combined with at least one compound d15; KXY, in each case combined with at least one compound d16; KXY, in each case combined with at least one compound d17; KXY, in each case combined with at least one compound d18; KXY, in each case combined with at least one compound d19; KXY, in each case combined with at least one compound d20; KXY, in each case combined with at least one compound d21; KXY, in each case combined with at least one compound d22; KXY, in each case combined with at least one compound d23; KXY, in each case combined with at least one compound d24; KXY, in each case combined with at least one compound d25; KXY, in each case combined with at least one compound d26; KXY, in each case combined with at least one compound d27; KXY, in each case combined with at least one compound d28; KXY, in each case combined with at least one compound d29; KXY, in each case combined with at least one compound d30; KXY, in each case combined with at least one compound d31; KXY, in each case combined with at least one compound d32; KXY, in each case combined with at least one compound d33; KXY, in each case combined with at least one compound d34; KXY, in each case combined with at least one compound d35; KXY, in each case combined with at least one compound d36; KXY, in each case combined with at least one compound d37; KXY, in each case combined with at least one compound d38; KXY, in each case combined with at least one compound d39; KXY, in each case combined with at least one compound d40; KXY, in each case combined with at least one compound d41; KXY, in each case combined with at least one compound d42; KXY, in each case combined with at least one compound d43; KXY, in each case combined with at least one compound d44; KXY, in each case combined with at least one compound d45; KXY, in each case combined with at least one compound d46; KXY, in each case combined with at least one compound d47; KXY, in each case combined with at least one compound d48; KXY, in each case combined with at least one compound d49; KXY, in each case combined with at least one compound d50; KXY, in each case combined with at least one compound d51; KXY, in each case combined with at least one compound d52; KXY, in each case combined with at least one compound d53; KXY, in each case combined with at least one compound d54; KXY, in each case combined with at least one compound d55; KXY, in each case combined with at least one compound d56; KXY, in each case combined with at least one compound d57; KXY, in each case combined with at least one compound d58; KXY, in each case combined with at least one compound d59; KXY, in each case combined with at least one compound d60; KXY, in each case combined with at least one compound d61; KXY, in each case combined with at least one compound d62; KXY, in each case combined with at least one compound d63; KXY, in each case combined with at least one compound d64; KXY, in each case combined with at least one compound d65; KXY, in each case combined with at least one compound d66; KXY, in each case combined with at least one compound d67; KXY, in each case combined with at least one compound d68; KXY, in each case combined with at least one compound d69; KXY, in each case combined with at least one compound d70; KXY, in each case combined with at least one compound d71; KXY, in each case combined with at least one compound d72; KXY, in each case combined with at least one compound d73; KXY, in each case combined with at least one compound d74; KXY, in each case combined with at least one compound d75; KXY, in each case combined with at least one compound d76; KXY, in each case combined with at least one compound d77; KXY, in each case combined with at least one compound d78; KXY, in each case combined with at least one compound d79; KXY, in each case combined with at least one compound d80; KXY, in each case combined with at least one compound d81; KXY, in each case combined with at least one compound d82; KXY, in each case combined with at least one compound d83; KXY, in each case combined with at least one compound d84; KXY, in each case combined with at least one compound d85; KXY, in each case combined with at least one module d86; KXY, in each case combined with at least one compound d87; KXY, in each case combined with at least one compound d88; KXY, in each case combined with at least one compound d89; KXY, in each case combined with at least one compound d90; KXY in each case combined with at least one compound d91; KXY, in each case combined with at least one compound d92; KXY, in each case combined with at least one compound d93; KXY, in each case combined with at least one compound d94; KXY, in each case combined with at least one compound d95; KXY, in each case combined with at least one module d96; KXY, in each case combined with at least one compound d97; KXY, in each case combined with at least one compound d98; KXY, in each case combined with at least one compound d99; KXY, in each case combined with at least one compound d100; KXY, in each case combined with at least one compound d101; KXY, in each case combined with at least one compound d102; KXY, in each case combined with at least one compound d103; KXY, in each case combined with at least one compound d104; KXY, in each case combined with at least one compound d105; KXY, in each case combined with at least one compound d106; KXY, in each case combined with at least one compound d107; KXY, in each case combined with at least one compound d108; KXY, in each case combined with at least one compound d109; KXY, in each case combined with at least one compound d110; KXY, in each case combined with at least one compound d111; KXY, in each case combined with at least one compound d112; KXY, in each case combined with at least one compound d113; KXY, in each case combined with at least one compound d114; KXY, in each case combined with at least one compound d115; KXY, in each case combined with at least one compound d116; KXY, in each case combined with at least one compound d117; KXY, in each case combined with at least one compound d118; KXY, in each case combined with at least one compound d119; KXY, in each case combined with at least one compound d120; KXY, in each case combined with at least one compound d121; KXY, in each case combined with at least one compound d122; KXY, in each case combined with at least one compound d123; KXY, in each case combined with at least one compound d124; KXY, in each case combined with at least one compound d125; KXY, in each case combined with at least one compound d126; KXY, in each case combined with at least one compound d127; KXY, in each case combined with at least one compound d128; KXY, in each case combined with at least one compound d129; KXY, in each case combined with at least one compound d130; KXY, in each case combined with at least one compound d131; KXY, in each case combined with at least one compound d132; KXY, in each case combined with at least one compound d133; KXY, in each case combined with at least one compound d134; KXY, in each case combined with at least one compound d135; KXY, in each case combined with at least one compound d136; KXY, in each case combined with at least one compound d137; KXY, in each case combined with at least one compound d138; KXY, in each case combined with at least one compound d139; KXY, in each case combined with at least one compound d140; KXY, in each case combined with at least one compound d141; KXY, in each case combined with at least one compound d142; KXY, in each case combined with at least one compound d143; KXY, in each case combined with at least one compound d144; KXY, in each case combined with at least one compound d145; KXY, in each case combined with at least one compound d146; KXY, in each case combined with at least one compound d147; KXY, in each case combined with at least one compound d148; KXY, in each case combined with at least one compound d149; KXY, in each case combined with at least one compound d150; KXY, in each case combined with at least one compound d151; KXY, in each case combined with at least one compound d152; KXY, in each case combined with at least one compound d153; KXY, in each case combined with at least one compound d154; KXY, in each case combined with at least one compound d155; KXY, in each case combined with at least one compound d156; KXY, in each case combined with at least one compound d157; KXY, in each case combined with at least one compound d158; KXY, in each case combined with at least one compound d159; KXY, in each case combined with at least one compound d160; KXY, in each case combined with at least one compound d161; KXY, in each case combined with at least one compound d162; KXY, in each case combined with at least one compound d163; KXY, in each case combined with at least one compound d164; KXY, in each case combined with at least one compound d165; KXY, in each case combined with at least one compound d166; KXY, in each case combined with at least one compound d167; KXY, in each case combined with at least one compound d168; KXY, in each case combined with at least one compound d169; or KXY, in each case combined with at least one compound d170;
wherein X is the combination of the at least one module (a) and the at least one module (b) and has the following meaning: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000. 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 0177, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1245, 1246, 1247, 1248, 1249, 1250, 1251, 1252, 1253, 1254, 1255, 1256, 1257, 1258, 1259, 1260, 1261, 1262, 1263, 1264, 1265, 1266, 1267, 1268, 1269, 1270, 1271, 1272, 1273, 1274, 1275, 1276, 1277, 1278, 1279, 1280, 1281, 1282, 1283, 1284, 1285, 1286, 1287, 1288, 1289, 1290, 1291, 1292, 1293, 1294, 1295, 1296, 1297, 1298, 1299, 1300, 1301, 1302, 1303, 1304, 1305, 1306, 1307, 1308, 1309, 1310, 1311, 1312, 1313, 1314, 1315, 1316, 1317, 1318, 1319, 1320, 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, 1349, 1350, 1351, 1352, 1353, 1354, 1355, 1356, 1357, 1358, 1359, 1360, 1361, 1362, 1363, 1364, 1365, 1366, 1367, 1368, 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, 1394, 1395, 1396, 1397, 1398, 1399, 1400, 1401, 1402, 1403, 1404, 1405, 1406, 1407, 1408, 1409, 1410, 1411, 1412, 1413, 1414, 1415, 1416, 1417, 1418, 1419, 1420, 1421, 1422, 1423, 1424, 1425, 1426, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, 1503, 1504, 1505, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1604, 1605, 1606, 1607, 1608, 1609, 1610, 1611, 1612, 1613, 1614, 1615, 1616, 1617, 1618, 1619, 1620, 1621, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1649, 1650, 1651, 1652, 1653, 1654, 1655, 1656, 1657, 1658, 1659, 1660, 1661, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, 1732, 1733, 1734, 1735, 1736, 1737, 1738, 1739, 1740, 1741, 1742, 1743, 1744, 1745, 1746, 1747, 1748, 1749, 1750, 4919, 4920, 4921, 4922, 4923, 4924, 4925, 4926, 4927, 4928, 4929, 4930, 4931, 4932, 4933, 4934, 4935, 4936, 4937, 4938, 4939, 4940, 4941, 4942, 4943, 4944, 4945, 4946, 4947, 4948, 4949, 4950, 4951, 4952, 4953, 4954, 4955, 4956, 4957, 4958, 4959, 4960, 4961, 4962, 4963, 4964, 4965, 4966, 4967, 4968, 4969, 4970, 4971, 4972, 4973, 4974, 4975, 4976, 4977, 4978, 4979, 4980, 4981, 4982, 4983, 4984, 4985, 4986, 4987, 4988, 4989, 4990, 4991, 4992, 4993, 4994, 4995, 4996, 4997, 4998, 4999, 5000, 5001, 5002, 5003, 5004, 5005, 5006, 5007, 5008, 5009, 5010, 5011, 5012, 5013, 5014, 5015, 5016, 5017, 5018, 5019, 5020, 5021, 5022, 5023, 5024, 5025, 5026, 5027, 5028, 5029, 5030, 5031, 5032, 5033, 5034, 5035, 5036, 5037, 5038, 5039, 5040, 5041, 5042, 5043, 5044, 5045, 5046, 5047, 5048, 5049, 5050, 5051, 5052, 5053, 5054, 5055, 5056, 5057, 5058, 5059, 5060, 5061, 5062, 5063, 5064, 5065, 5066, 5067, 5068, 5069, 5070, 5071, 5072, 5073, 5074, 5075, 5076, 5077, 5078, 5079, 5080, 5081, 5082, 5083, 5084, 5085, 5086, 5087, 5088, 5089, 5090, 5091, 5092, 5093, 5094, 5095, 5096, 5097, 5098, 5099, 5100, 5101, 5102, 5103, 5104, 5105, 5106, 5107, 5108, 5109, 5110, 5111, 5112, 5113, 5114, 5115, 5116, 5117, 5118, 5119, 5120, 5121, 5122, 5123, 5124, 5125, 5126, 5127, 5128, 5129, 5130, 5131, 5132, 5133, 5134, 5135, 5136, 5137, 5138, 5139, 5140, 5141, 5142, 5143, 5144, 5145, 5146, 5147, 5148, 5149, 5150, 5151, 5152, 5153, 5154, 5155, 5156, 5157, 5158, 5159, 5160, 5161, 5162, 5163, 5164, 5165, 5166, 5167, 5168, 5169, 5170, 5171, 5172, 5173, 5174, 5175, 5176, 5177, 5178, 5179, 5180, 5181, 5182, 5183, 5184, 5185, 5186, 5187, 5188, 5189, 5190, 5191, 5192, 5193, 5194, 5195, 5196, 5197, 5198, 5199, 5200, 5201, 5202, 5203, 5204, 5205, 5206, 5207, 5208, 5209, 5210, 5211, 5212, 5213, 5214, 5215, 5216, 5217, 5218, 5219, 5220, 5221, 5222, 5223, 5224, 5225, 5226, 5227, 5228, 5229, 5230, 5231, 5232, 5233, 5234, 5235, 5236, 5237, 5238, 5239, 5240, 5241, 5242, 5243, 5244, 5245, 5246, 5247, 5248, 5249, 5250, 5251, 5252, 5253, 5254, 5255, 5256, 5257, 5258, 5259, 5260, 5261, 5262, 5263, 5264, 5265, 5266, 5267, 5268, 5269, 5270, 5271, 5272, 5273, 5274, 5275, 5276, 5277, 5278, 5279, 5280, 5281, 5282, 5283, 5284, 5285, 5286, 5287, 5288, 5289, 5290, 5291, 5292, 5293, 5294, 5295, 5296, 5297, 5298, 5299, 5300, 5301, 5302, 5303, 5304, 5305, 5306, 5307, 5308, 5309, 5310, 5311, 5312, 5313, 5314, 5315, 5316, 5317, 5318, 5319, 5320, 5321, 5322, 5323, 5324, 5325, 5326, 5327, 5328, 5329, 5330, 5331, 5332, 5333, 5334, 5335, 5336, 5337, 5338, 5339, 5340, 5341, 5342, 5343, 5344, 5345, 5346, 5347, 5348, 5349, 5350, 5351, 5352, 5353, 5354, 5355, 5356, 5357, 5358, 5359, 5360, 5361, 5362, 5363, 5364, 5365, 5366, 5367, 5368, 5369, 5370, 5371, 5372, 5373, 5374, 5375, 5376, 5377, 5378, 5379, 5380, 5381, 5382, 5383, 5384, 5385, 5386, 5387, 5388, 5389, 5390, 5391, 5392, 5393, 5394, 5395, 5396, 5397, 5398, 5399, 5400, 5401, 5402, 5403, 5404, 5405, 5406, 5407, 5408, 5409, 5410, 5411, 5412, 5413, 5414, 5415, 5416, 5417, 5418, 5419, 5420, 5421, 5422, 5423, 5424, 5425, 5426, 5427, 5428, 5429, 5430, 5431, 5432, 5433, 5434, 5435, 5436, 5437, 5438, 5439, 5440, 5441, 5442, 5443, 5444, 5445, 5446, 5447, 5448, 5449, 5450, 5451, 5452, 5453, 5454, 5455, 5456, 5457, 5458, 5459, 5460, 5461, 5462, 5463, 5464, 5465, 5466, 5467, 5468, 5469, 5470, 5471, 5472, 5473, 5474, 5475, 5476, 5477, 5478, 5479, 5480, 5481, 5482, 5483, 5484, 5485, 5486, 5487, 5488, 5489, 5490, 5491, 5492, 5493, 5494, 5495, 5496, 5497, 5498, 5499, 5500, 5501, 5502, 5503, 5504, 5505, 5506, 5507, 5508, 5509, 5510, 5511, 5512, 5513, 5514, 5515, 5516, 5517, 5518, 5519, 5520, 5521, 5522, 5523, 5524, 5525, 5526, 5527, 5528, 5529, 5530, 5531, 5532, 5533, 5534, 5535, 5536, 5537, 5538, 5539, 5540, 5541, 5542, 5543, 5544, 5545, 5546, 5547, 5548, 5549, 5550, 5551, 5552, 5553, 5554, 5555, 5556, 5557, 5558, 5559, 5560, 5561, 5562, 5563, 5564, 5565, 5566, 5567, 5568, 5569, 5570, 5571, 5572, 5573, 5574, 5575, 5576, 5577, 5578, 5579, 5580, 5581, 5582, 5583, 5584, 5585, 5586, 5587, 5588, 5589, 5590, 5591, 5592, 5593, 5594, 5595, 5596, 5597, 5598, 5599, 5600, 5601, 5602, 5603, 5604, 5605, 5606, 5607, 5608, 5609, 5610, 5611, 5612, 5613, 5614, 5615, 5616, 5617, 5618, 5619, 5620, 5621, 5622, 5623, 5624, 5625, 5626, 5627, 5628, 5629, 5630, 5631, 5632, 5633, 5634, 5635, 5636, 5637, 5638, 5639, 5640, 5641, 5642, 5643, 5644, 5645, 5646, 5647, 5648, 5649, 5650, 5651, 5652, 5653, 5654, 5655, 5656, 5657, 5658, 5659, 5660, 5661, 5662, 5663, 5664, 5665, 5666, 5667, 5668, 5669, 5670, 5671, 5672, 5673, 5674, 5675, 5676, 5677, 5678, 5679, 5680, 5681, 5682, 5683, 5684, 5685, 5686, 5687, 5688, 5689, 5690, 5691, 5692, 5693, 5694, 5695, 5696, 5697, 5698, 5699, 5700, 5701, 5702, 5703, 5704, 5705, 5706, 5707, 5708, 5709, 5710, 5711, 5712, 5713, 5714, 5715, 5716, 5717, 5718, 5719, 5720, 5721, 5722, 5723, 5724, 5725, 5726, 5727, 5728, 5729, 5730, 5731, 5732, 5733, 5734, 5735, 5736, 5737, 5738, 5739, 5740, 5741, 5742, 5743, 5744, 5745, 5746, 5747, 5748, 5749, 5750, 5751, 5752, 5753, 5754, 5755, 5756, 5757, 5758, 5759, 5760, 5761, 5762, 5763, 5764, 5765, 5766, 5767, or 5768, wherein Y is the at least one module (c) and has the following meaning: c1, c2, c3, c4, c5, c6, c7, c8, c9, c10, c11, c12, c13, c14, c15, c16, c17, c18, c19, c20, c21, c22, c23, c24, c25, c26, c27, c28, c29, c30, c31, c32, c33, c34, c35, c36, c37, c38, c39, c40, c41, c42, c43, c44, c45, c46, c47, c48, c49, c50, c51, c52, c53, c54, c55, c56, c57, c58, c59, c60, c61, c62, c63, c64, c65, c66, c67, c68, c69, c70, c71, c72, c73, c74, c75, c76, c77, c78, c79, c80, c81, c82, c83, c84, c85, c86, c87, c88, c89, c90, c91, c92, c93, c94, c95, c96, c97, c98, c99, c100, c101, c102, c103, c104, c105, c106, c107, c108, c109, c110, c111, c112, c113, c114, c115, c116, c117, c118, c119, c120, c121, c122, c123, c124, c125, c126, c127, c128, c129, c130, c131, c132, c133, c134, c135, c136, c137, c138, c139, c140, c141, c142, c143, c144, c145, c146, c147, c148, c149, c150, c151, c152, c153, c154, c155, c156, c157, c158, c159, c160, c161, c162, c163, c164, c165, c166, c167, c168, c169, c170, c171, c172, c173, c174, c175, c176, c177, c178, c179, c180, c181, c182, c183, c184, c185, c186, c187, c188, c189, c190, c191, c192, c193, c194, c195, c196, c197, c198, c199, c200, c201, c202, c203, c204, c205, c206, c207, c208, c209, c210, or c211, and wherein the at least one module (a), the at least one module (b), the at least one module (c), and the at least one compound (d) are linked to each other in any arrangement and stoichiometry.

Preferably, the conjugate comprises, essentially consists of, consists of or contains:

(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
wherein the at least one module (a) is a ricin B-subunit and wherein the at least one module (a), the at least one module (b), and the at least one module (c), and the at least one compound (d) are linked to each other in any arrangement and stoichiometry.

Preferably, the conjugate comprises, essentially consists of, consists of or contains:

(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
wherein the at least one module (a) is a cholera toxin B-subunit and wherein the at least one module (a), the at least one module (b), and the at least one module (c), and the at least one compound (d) are linked to each other in any arrangement and stoichiometry.

Preferably, the conjugate comprises, essentially consists of, consists of or contains:

(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
wherein the at least one module (a) is a Shiga toxin B-subunit and wherein the at least one module (a), the at least one module (b), and the at least one module (c), and the at least one compound (d) are linked to each other in any arrangement and stoichiometry.

Preferably, the conjugate comprises, essentially consists of, consists of or contains:

(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
wherein the at least one module (a) is a viscumin toxin B-subunit and wherein the at least one module (a), the at least one module (b), and the at least one module (c), and the at least one compound (d) are linked to each other in any arrangement and stoichiometry.

Preferably, the conjugate comprises, essentially consists of, consists of or contains:

(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
wherein the at least one module (a) is a volkensin toxin B-subunit and wherein the at least one module (a), the at least one module (b), and the at least one module (c), and the at least one compound (d) are linked to each other in any arrangement and stoichiometry.

Preferably, the conjugate comprises, essentially consists of, consists of or contains:

(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
wherein the at least one module (a) is a pertussis toxin B-subunit and wherein the at least one module (a), the at least one module (b), and the at least one module (c), and the at least one compound (d) are linked to each other in any arrangement and stoichiometry.

Preferably, the conjugate comprises, essentially consists of, consists of or contains:

(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
wherein the at least one module (a) is an E. coli heat labile enterotoxin LT B-subunit and wherein the at least one module (a), the at least one module (b), and the at least one module (c), and the at least one compound (d) are linked to each other in any arrangement and stoichiometry.

Preferably, the conjugate comprises, essentially consists of, consists of or contains:

(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
wherein the at least one module (a) is an E. coli subtilase cytotoxin B-subunit and wherein the at least one module (a), the at least one module (b), and the at least one module (c), and the at least one compound (d) are linked to each other in any arrangement and stoichiometry.

Preferably, the conjugate comprises, essentially consists of, consists of or contains:

(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
wherein the at least one module (a) is a tetanus toxin C-fragment and wherein the at least one module (a), the at least one module (b), and the at least one module (c), and the at least one compound (d) are linked to each other in any arrangement and stoichiometry.

Preferably, the conjugate comprises, essentially consists of, consists of or contains:

(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
wherein the at least one module (a) is an AMF protein or peptide and wherein the at least one module (a), the at least one module (b), and the at least one module (c), and the at least one compound (d) are linked to each other in any arrangement and stoichiometry.

Preferably, the conjugate comprises, essentially consists of, consists of or contains:

(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
wherein the at least one module (a) is an SUMF protein or peptide and wherein the at least one module (a), the at least one module (b), and the at least one module (c), and the at least one compound (d) are linked to each other in any arrangement and stoichiometry.

Specific module (b)+generic (a), (c), and (d):

Preferably, the conjugate comprises, essentially consists of, consists of or contains:

(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
wherein the at least one module (b) is a protein or peptide that comprises, consists essentially of, consists of or contains an amino acid sequence of SEQ ID NO: 24, and wherein the at least one module (a), the at least one module (b), and the at least one module (c), and the at least one compound (d) are linked to each other in any arrangement and stoichiometry.

Preferably, the conjugate comprises, essentially consists of, consists of or contains:

(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
wherein the at least one module (b) is a protein or peptide that comprises, consists essentially of, consists of or contains an amino acid sequence of SEQ ID NO: 25, and wherein the at least one module (a), the at least one module (b), and the at least one module (c), and the at least one compound (d) are linked to each other in any arrangement and stoichiometry.

Preferably, the conjugate comprises, essentially consists of, consists of or contains:

(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
wherein the at least one module (b) is a protein or peptide that comprises, consists essentially of, consists of or contains an amino acid sequence of SEQ ID NO: 26, and wherein the at least one module (a), the at least one module (b), and the at least one module (c), and the at least one compound (d) are linked to each other in any arrangement and stoichiometry.

Specific module (c)+generic (a), (b), and (d):

Preferably, the conjugate comprises, essentially consists of, consists of or contains:

(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
wherein the at least one module (c) is a COX2 protein or peptide, and wherein the at least one module (a), the at least one module (b), and the at least one module (c), and the at least one compound (d) are linked to each other in any arrangement and stoichiometry. Preferably the COX2 protein or peptide comprises, consists essentially of, consists of or contains an amino acid sequence that comprises SEQ ID NO: 43 or SEQ ID NO: 44.

Preferably, the conjugate comprises, essentially consists of, consists of or contains:

(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
wherein the at least one module (c) is an IgM(μ) protein or peptide, and wherein the at least one module (a), the at least one module (b), and the at least one module (c), and the at least one compound (d) are linked to each other in any arrangement and stoichiometry. Preferably the IgM(μ) protein or peptide comprises, consists essentially of, consists of or contains an amino acid sequence that comprises SEQ ID NO: 53.

Preferably, the conjugate comprises, essentially consists of, consists of or contains:

(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
wherein the at least one module (c) is a Sgk1 protein or peptide, and wherein the at least one module (a), the at least one module (b), and the at least one module (c), and the at least one compound (d) are linked to each other in any arrangement and stoichiometry. Preferably the Sgk1 protein or peptide comprises, consists essentially of, consists of or contains an amino acid sequence that comprises SEQ ID NO: 66, SEQ ID NO: 72, or SEQ ID NO: 73.

Preferably, the conjugate comprises, essentially consists of, consists of or contains:

(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
wherein the at least one module (c) is an AChE protein or peptide, and wherein the at least one module (a), the at least one module (b), and the at least one module (c), and the at least one compound (d) are linked to each other in any arrangement and stoichiometry. Preferably the AChE protein or peptide comprises, consists essentially of, consists of or contains an amino acid sequence selected from the group consisting of SEQ ID NO: 280, SEQ ID NO: 281, SEQ ID NO: 282, SEQ ID NO: 283, SEQ ID NO: 284, SEQ ID NO: 285, SEQ ID NO: 286, SEQ ID NO: 287, SEQ ID NO: 288, and SEQ ID NO: 289.

Preferably, the conjugate comprises, essentially consists of, consists of or contains:

(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
wherein the at least one module (c) is a mutant RTA protein or peptide, and wherein the at least one module (a), the at least one module (b), and the at least one module (c), and the at least one compound (d) are linked to each other in any arrangement and stoichiometry. Preferably the mutant RTA protein or peptide comprises, consists essentially of, consists of or contains an A1-subunit comprising a G247W substitution, an S250P substitution, a G247Q substitution, a W246R substitution, an E212D substitution, an E212K substitution, an 1287R substitution, an R215Q substitution, an E212Q substitution, a Y115S substitution, a Y158S substitution, a deletion of amino acids 110-115 (DVTNAY), or a Y115AN111M double substitution (RiVax), and wherein the numerical position of the A1-subunit's amino acid substitution or deletion is indicated according to the reference sequence Uniprot sequence P02879 that comprises the full length ricin amino acid sequence, including the signal peptide. Preferably, the mutant RTA protein or peptide lacks a signal peptide.

Preferably, the conjugate comprises, essentially consists of, consists of or contains:

(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
wherein the at least one module (c) is a mutant CTA protein or peptide, and wherein the at least one module (a), the at least one module (b), and the at least one module (c), and the at least one compound (d) are linked to each other in any arrangement and stoichiometry. Preferably the mutant CTA protein or peptide comprises, consists essentially of, consists of or contains an A1-subunit comprising or consisting of an amino acid sequence of SEQ ID NO: 154, SEQ ID NO: 155, SEQ ID NO: 156, an E112K substitution, an S61F substitution, or an E29H substitution. Preferably, the mutated cholera toxin A-subunit for use as a module (c) lacks a signal peptide.

Preferably, the conjugate comprises, essentially consists of, consists of or contains:

(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
wherein the at least one module (c) is a mutant Shiga toxin A-subunit protein or peptide, and wherein the at least one module (a), the at least one module (b), and the at least one module (c), and the at least one compound (d) are linked to each other in any arrangement and stoichiometry.

Preferably, the conjugate comprises, essentially consists of, consists of or contains:

(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
wherein the at least one module (c) is a mutant PTA protein or peptide, and wherein the at least one module (a), the at least one module (b), and the at least one module (c), and the at least one compound (d) are linked to each other in any arrangement and stoichiometry. Preferably the mutant PTA protein or peptide comprises, consists essentially of, consists of or contains a pertussis toxin A-subunit comprising an R43 amino acid deletion, an R43K substitution, an R43H substitution, a five (5) amino acid deletion of R43 to R47, an R92E substitution, a W60A substitution, an H69A substitution, a C75A substitution, an E163 amino acid deletion, an E163G substitution, an E163Q substitution, an E163D substitution, an E163N substitution, an E163K substitution, an E163H substitution, an E163P substitution, an E163S substitution, an E163G/Y164A double substitution, an E163G/Y164F double substitution, a C75A/E163G double substitution, an R43K/E163G double substitution, an R43K/R92E/E163G triple substitution, or an R92E/E163G double substitution, wherein the numerical position of the amino acid deletion or substitution is indicated according to the reference sequence Uniprot sequence PO4977. While the reference sequence used here (i.e., Uniprot sequence P04977) to identify the location of these mutations in the pertussis toxin A-subunit comprises a signal peptide, the mutated pertussis toxin A-subunit protein or peptide for use as a module (c) of the invention preferably lacks this signal peptide.

Preferably, the conjugate comprises, essentially consists of, consists of or contains:

(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
wherein the at least one module (c) is a mutant abrin toxin A-subunit protein or peptide, and wherein the at least one module (a), the at least one module (b), and the at least one module (c), and the at least one compound (d) are linked to each other in any arrangement and stoichiometry. Preferably the mutant abrin toxin A-subunit protein or peptide is a mutated abrin a toxin A-subunit protein or peptide that comprises, consists essentially of, consists of or contains an E164A/R167L double substitution, an E164A substitution, or an R167L substitution, wherein the numerical position of the mutated Abrin a A-subunit's amino acid substitution is indicated according to the reference sequence Uniprot P11140 (ABRA_ABRPR; abrin a). Preferably, the mutated abrin toxin A-subunit protein or peptide and mutated abrin a toxin A-subunit for use as a module (c) of the invention preferably lack a signal peptide.

Preferably, the conjugate comprises, essentially consists of, consists of or contains:

(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
wherein the at least one module (c) is a mutant E. coli subtilase cytotoxin A-subunit protein or peptide, and wherein the at least one module (a), the at least one module (b), and the at least one module (c), and the at least one compound (d) are linked to each other in any arrangement and stoichiometry. Preferably the mutant E. coli subtilase cytotoxin A-subunit protein or peptide comprises, consists essentially of, consists of or contains an S272A substitution, wherein the numerical position of the mutated E. coli subtilase cytotoxin A-subunit's amino acid substitution is indicated according to the reference sequence http://www.uniprot.org/uniprot/Q6EZC2. Preferably, the mutated E. coli subtilase cytotoxin A-subunit protein or peptide for use as a module (c) of the invention preferably lacks a signal peptide.

Preferably, the conjugate comprises, essentially consists of, consists of or contains:

(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
wherein the at least one module (c) is a mutant LT A-subunit protein or peptide, and wherein the at least one module (a), the at least one module (b), and the at least one module (c), and the at least one compound (d) are linked to each other in any arrangement and stoichiometry. Preferably the mutant LT A-subunit protein or peptide comprises, consists essentially of, consists of or contains a S81K substitution, an A90R substitution, an S81Y substitution, a deletion of amino acids 128-130, or an E130K substitution, wherein the numerical position of the mutated LT A-subunit's amino acid substitution or deletion is indicated according to the reference sequence Uniprot sequence P43530 containing a signal peptide. While the reference sequence used here (i.e., Uniprot sequence P43530) to identify the location of these mutations in the LT A-subunit comprises a signal peptide, the mutated LT A-subunit protein or peptide for use as a module (c) of the invention preferably lacks this signal peptide.

Preferably, the conjugate comprises, essentially consists of, consists of or contains:

(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
wherein the at least one compound (d) is a nucleic acid, and wherein the at least one module (a), the at least one module (b), and the at least one module (c), and the at least one compound (d) are linked to each other in any arrangement and stoichiometry.

Preferably, a conjugate comprises, essentially consists of, consists of or contains:

(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
wherein the at least one compound (d) is a single stranded RNA molecule, and wherein the at least one module (a), the at least one module (b), and the at least one module (c), and the at least one compound (d) are linked to each other in any arrangement and stoichiometry.

Preferably, a conjugate comprises, essentially consists of, consists of or contains:

(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
wherein the at least one compound (d) is a double stranded RNA molecule, and wherein the at least one module (a), the at least one module (b), and the at least one module (c), and the at least one compound (d) are linked to each other in any arrangement and stoichiometry.

Preferably, a conjugate comprises, essentially consists of, consists of or contains:

(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
wherein the at least one compound (d) is an siRNA molecule, and wherein the at least one module (a), the at least one module (b), and the at least one module (c), and the at least one compound (d) are linked to each other in any arrangement and stoichiometry.

Preferably, a conjugate comprises, essentially consists of, consists of or contains:

(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
wherein the at least one compound (d) is an shRNA molecule, and wherein the at least one module (a), the at least one module (b), and the at least one module (c), and the at least one compound (d) are linked to each other in any arrangement and stoichiometry.

Preferably, a conjugate comprises, essentially consists of, consists of or contains:

(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
wherein the at least one compound (d) is an miRNA molecule, and wherein the at least one module (a), the at least one module (b), and the at least one module (c), and the at least one compound (d) are linked to each other in any arrangement and stoichiometry.

### Particularly Preferred Conjugates

Particularly preferred conjugates of the present invention include: an RTB-KDEL-Cox2 conjugate comprising a15+b20+(one of c14-c22); an RTB-KDEL-Cox2 conjugate comprising a15+b20+c17; an RTB-KDEL-Cox2 conjugate comprising a15+b20+c18; an RTB-KDEL-IgM(μ) conjugate comprising a15+b20+(one of c23-c30); an RTB-KDEL-IgM(μ) conjugate comprising a15+b20+c27; an RTB-KDEL-IgM(μ) conjugate comprising a15+b20+c28; an RTB-KDEL-Sgk1 conjugate comprising a15+b20+(one of c31-c51); an RTB-KDEL-Sgk1 conjugate comprising a15+b20+c40; an RTB-KDEL-Sgk1 conjugate comprising a15+b20+c46; an RTB-KDEL-Sgk1 conjugate comprising a15+b20+c47; an RTB-KDEL-AChE conjugate comprising a15+b20+(one of c202-c211); a CTB-KDEL-Cox2 conjugate comprising a17+b20+(one of c14-c22); a CTB-KDEL-Cox2 conjugate comprising a17+b20+c17; a CTB-KDEL-Cox2 conjugate comprising a17+b20+c18; a CTB-KDEL-IgM(μ) conjugate comprising a17+b20+(one of c23-c30); a CTB-KDEL-IgM(μ) conjugate comprising a17+b20+c27; a CTB-KDEL-IgM(μ) conjugate comprising a17+b20+c28; a CTB-KDEL-Sgk1 conjugate comprising a17+b20+(one of c31-c51); a CTB-KDEL-Sgk1 conjugate comprising a17+b20+c40; aCTB-KDEL-Sgk1 conjugate comprising a17+b20+c46; a CTB-KDEL-Sgk1 conjugate comprising a17+b20+c47; a CTB-KDEL-AChE conjugate comprising a17+b20+(one of c202-c211); an STB-KDEL-Cox2 conjugate comprising (one of a21, a23-a32, or a71)+b20+(one of c14-c22); an STB-KDEL-Cox2 conjugate comprising (one of a21, a23-a32, or a71)+b20+c17; an STB-KDEL-Cox2 conjugate comprising (one of a21, a23-a32, or a71)+b20+c18; an STB-KDEL-IgM(μ) conjugate comprising (one of a21, a23-a32, or a71)+b20+(one of c23-c30); an STB-KDEL-IgM(μ) conjugate comprising (one of a21, a23-a32, or a71)+b20+c27; an STB-KDEL-IgM(μ) conjugate comprising (one of a21, a23-a32, or a71)+b20+c28; an STB-KDEL-Sgk1 conjugate comprising (one of a21, a23-a32, or a71)+b20+(one of c31-c51); an STB-KDEL-Sgk1 conjugate comprising (one of a21, a23-a32, or a71)+b20+c40; an STB-KDEL-Sgk1 conjugate comprising (one of a21, a23-a32, or a71)+b20+c46; an STB-KDEL-Sgk1 conjugate comprising a21+b20+c47; an STB-KDEL-AChE conjugate comprising (one of a21, a23-a32, or a71)+b20+(one of c202-c211); an AMF-KDEL-Cox2 conjugate comprising a54+b20+(one of c14-c22); an AMF-KDEL-Cox2 conjugate comprising a54+b20+c17; an AMF-KDEL-Cox2 conjugate comprising a54+b20+c18; an AMF-KDEL-IgM(μ) conjugate comprising a54+b20+(one of c23-c30); an AMF-KDEL-IgM(μ) conjugate comprising a54+b20+c27; an AMF-KDEL-IgM(μ) conjugate comprising a54+b20+c28; an AMF-KDEL-Sgk1 conjugate comprising a54+b20+(one of c31-c51); an AMF-KDEL-Sgk1 conjugate comprising a54+b20+c40; an AMF-KDEL-Sgk1 conjugate comprising a54+b20+c46; an AMF-KDEL-Sgk1 conjugate comprising a54+b20+c47; an AMF-KDEL-AChE conjugate comprising a54+b20+(one of c202-c211); a Viscumin B-KDEL-Cox conjugate comprising a43+b20+(one of c14-c22); a Viscumin B-KDEL-Cox2 conjugate comprising a43+b20+c17; a Viscumin B-KDEL-Cox2 conjugate comprising a43+b20+c18; a Viscumin B-KDEL-IgM(μ) conjugate comprising a43+b20+(one of c23-c30); a Viscumin B-KDEL-IgM(μ) conjugate comprising a43+b20+c27; a Viscumin B-KDEL-IgM(μ) conjugate comprising a43+b20+c28; a Viscumin B-KDEL-Sgk1 conjugate comprising a43+b20+(one of c31-c51); a Viscumin B-KDEL-Sgk1 conjugate comprising a43+b20+c40; a Viscumin B-KDEL-Sgk1 conjugate comprising a43+b20+c46; a Viscumin B-KDEL-Sgk1 conjugate comprising a43+b20+c47; a Viscumin B-KDEL-AChE conjugate comprising a43+b20+(one of c202-c211); a Volkensin B-KDEL-Cox2 conjugate comprising a42+b20+(one of c14-c22); a Volkensin B-KDEL-Cox2 conjugate comprising a42+b20+c17; a Volkensin B-KDEL-Cox2 conjugate comprising a42+b20+c18; a Volkensin B-KDEL-IgM(μ) conjugate comprising a42+b20+(one of c23-c30); a Volkensin B-KDEL-IgM(μ) conjugate comprising a42+b20+c27; a Volkensin B-KDEL-IgM(μ) conjugate comprising a42+b20+c28; a Volkensin B-KDEL-Sgk1 conjugate comprising a42+b20+(one of c31-c51); a Volkensin B-KDEL-Sgk1 conjugate comprising a42+b20+c40; a Volkensin B-KDEL-Sgk1 conjugate comprising a42+b20+c46; a Volkensin B-KDEL-Sgk1 conjugate comprising a42+b20+c47; a Volkensin B-KDEL-AChE conjugate comprising a42+b20+(one of c202-c211); a PTB-KDEL-Cox2 conjugate comprising a40+b20+(one of c14-c22); a PTB-KDEL-Cox2 conjugate comprising a40+b20+c17; a PTB-KDEL-Cox2 conjugate comprising a40+b20+c18; a PTB-KDEL-IgM(μ) conjugate comprising a40+b20+(one of c23-c30); a PTB-KDEL-IgM(μ) conjugate comprising a40+b20+c27; a PTB-KDEL-IgM(μ) conjugate comprising a40+b20+c28; a PTB-KDEL-Sgk1 conjugate comprising a40+b20+(one of c31-c51); a PTB-KDEL-Sgk1 conjugate comprising a40+b20+c40; a PTB-KDEL-Sgk1 conjugate comprising a40+b20+c46; a PTB-KDEL-Sgk1 conjugate comprising a40+b20+c47; a PTB-KDEL-AChE conjugate comprising a40+b20+(one of c202-c211); an LT B-KDEL-Cox2 conjugate comprising (one of a33-a35)+b20+(one of c14-c22); an LT B-KDEL-Cox2 conjugate comprising (one of a33-a35)+b20+c17; an LT B-KDEL-Cox2 conjugate comprising (one of a33-a35)+b20+c18; an LT B-KDEL-IgM(μ) conjugate comprising (one of a33-a35)+b20+(one of c23-c30); an LT B-KDEL-IgM(μ) conjugate comprising (one of a33-a35)+b20+c27; an LT B-KDEL-IgM(μ) conjugate comprising (one of a33-a35)+b20+c28; an LT B-KDEL-Sgk1 conjugate comprising (one of a33-a35)+b20+(one of c31-c51); an LT B-KDEL-Sgk1 conjugate comprising (one of a33-a35)+b20+c40; an LT B-KDEL-Sgk1 conjugate comprising (one of a33-a35)+b20+c46; an LT B-KDEL-Sgk1 conjugate comprising (one of a33-a35)+b20+c47; an LT B-KDEL-AChE conjugate comprising (one of a33-a35)+b20+(one of c202-c211); an E. coli subtilase B-KDEL-Cox2 conjugate comprising a45+b20+(one of c14-c22); an E. coli subtilase B-KDEL-Cox2 conjugate comprising a45+b20+c17; an E. coli subtilase B-KDEL-Cox2 conjugate comprising a45+b20+c18; an E. coli subtilase B-KDEL-IgM(μ) conjugate comprising a45+b20+(one of c23-c30); an E. coli subtilase B-KDEL-IgM(μ) conjugate comprising a45+b20+c27; an E. coli subtilase B-KDEL-IgM(μ) conjugate comprising a45+b20+c28; an E. coli subtilase B-KDEL-Sgk1 conjugate comprising a45+b20+(one of c31-c51); an E. coli subtilase B-KDEL-Sgk1 conjugate comprising a45+b20+c40; an E. coli subtilase B-KDEL-Sgk1 conjugate comprising a45+b20+c46; an E. coli subtilase B-KDEL-Sgk1 conjugate comprising a45+b20+c47; an E. coli subtilase B-KDEL-AChE conjugate comprising a45+b20+(one of c202-c211); a Tetanus C-fragment-KDEL-Cox2 conjugate comprising a46+b20+(one of c14-c22); a Tetanus C-fragment-KDEL-Cox2 conjugate comprising a46+b20+c17; a Tetanus C-fragment-KDEL-Cox2 conjugate comprising a46+b20+c18; a Tetanus C-fragment-KDEL-IgM(μ) conjugate comprising a46+b20+(one of c23-c30); a Tetanus C-fragment-KDEL-IgM(μ) conjugate comprising a46+b20+c27; a Tetanus C-fragment-KDEL-IgM(μ) conjugate comprising a46+b20+c28; a Tetanus C-fragment-KDEL-Sgk1 conjugate comprising a46+b20+(one of c31-c51); a Tetanus C-fragment-KDEL-Sgk1 conjugate comprising a46+b20+c40; a Tetanus C-fragment-KDEL-Sgk1 conjugate comprising a46+b20+c46; a Tetanus C-fragment-KDEL-Sgk1 conjugate comprising a46+b20+c47; a Tetanus C-fragment-KDEL-AChE conjugate comprising a46+b20+(one of c202-c211); an SUMF-KDEL-Cox2 conjugate comprising a55+b20+(one of c14-c22); an SUMF-KDEL-Cox2 conjugate comprising a55+b20+c17; an SUMF-KDEL-Cox2 conjugate comprising a55+b20+c18; an SUMF-KDEL-IgM(μ) conjugate comprising a55+b20+(one of c23-c30); an SUMF-KDEL-IgM(μ) conjugate comprising a55+b20+c27; an SUMF-KDEL-IgM(μ) conjugate comprising a55+b20+c28; an SUMF-KDEL-Sgk1 conjugate comprising a55+b20+(one of c31-c51); an SUMF-KDEL-Sgk1 conjugate comprising a55+b20+c40; an SUMF-KDEL-Sgk1 conjugate comprising a55+b20+c46; an SUMF-KDEL-Sgk1 conjugate comprising a55+b20+c47, and an SUMF-KDEL-AChE conjugate comprising a55+b20+(one of c202-c211). Preferably, these preferred conjugates of the invention further comprise at least one compound (d). More preferably, these preferred conjugates of the invention further comprise at least one compound (d) selected from the group consisting of a nucleic acid, a DNA molecule, an RNA molecule, a single stranded RNA molecule, a double stranded RNA molecule, an siRNA molecule, an shRNA molecule, an miRNA molecule, a protein, and a peptide, most preferably a siRNA.

Preferably, the mutant toxin protein or peptide of use in the present invention as a module (c) or as part of a multi-module protein or peptide comprises a mutation that reduces or abolishes the toxin protein's or toxin peptide's toxicity while maintaining its ERAD substrate functionality. In this regard, particularly preferred conjugates of the present invention include: an RTB-KDEL-mRTA conjugate comprising a15+b20+(one of c78 or c79); an STB-KDEL-mSTA conjugate comprising a21+b20+(one of c84 or c85); an RTB-KDEL-mPTA conjugate comprising a15+b20+c118; an CTB-KDEL-mPTA conjugate comprising a17+b20+c118; an STB-KDEL-mPTA conjugate comprising a21+b20+c118; an RTB-KDEL-mViscumin A conjugate comprising a15+b20+c124; an CTB-KDEL-mViscumin A conjugate comprising a17+b20+c124; an STB-KDEL-mViscumin A conjugate comprising a21+b20+c124; an RTB-KDEL-mVolkensin A conjugate comprising a15+b20+c122; an CTB-KDEL-mVolkensin A conjugate comprising a17+b20+c122; an STB-KDEL-mVolkensin A conjugate comprising a21+b20+c122; an RTB-KDEL-mLTA conjugate comprising a15+b20+(one of c107-c110); an CTB-KDEL-mLTA conjugate comprising a17+b20+(one of c107-c110); an STB-KDEL-mLTA conjugate comprising a21+b20+(one of c107-c110); an RTB-KDEL-m E. coli Subtilase A conjugate comprising a15+b20+c129; an CTB-KDEL-m E. coli Subtilase A conjugate comprising a17+b20+c129; an STB-KDEL-m E. coli Subtilase A conjugate comprising a21+b20+c129; an RTB-KDEL-Sambucus protein conjugate comprising a15+b20+(one of c134-c140); an CTB-KDEL-Sambucus protein conjugate comprising a17+b20+(one of c134-c140); an STB-KDEL-Sambucus protein conjugate comprising a21+b20+(one of c134-c140); an RTB-KDEL-mCinnamomin A conjugate comprising a15+b20+(one of c131-c133); an CTB-KDEL-mCinnamomin A conjugate comprising a17+b20+(one of c131-c133); and an STB-KDEL-mCinnamomin A conjugate comprising a21+b20+(one of c131-c133). Preferably, these preferred conjugates of the invention further comprise at least one compound (d). More preferably, these preferred conjugates of the invention further comprise at least one compound (d) selected from the group consisting of a nucleic acid, a DNA molecule, an RNA molecule, a single stranded RNA molecule, a double stranded RNA molecule, an siRNA molecule, an shRNA molecule, an miRNA molecule, a protein, and a peptide.

(a+b) Multi-Module Protein or Peptide

Preferably, a conjugate of the delivery system according to the second aspect comprises, essentially consists of, or consists of or contains:
(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
   wherein the at least one module (a) and the at least one module (b) are comprised or contained within a [module (a)+module (b)] protein or peptide, and wherein the [module (a)+module (b)] protein or peptide, the at least one module (c), and the at least one compound (d) are linked to each other in any arrangement. The conjugates of the present invention optionally comprise a nuclear localization signal.

Preferably, the [module (a)+module (b)] protein or peptide comprises, consists essentially of, consists of or contains a mutated holo-toxin having reduced or no toxicity (ab1), a non-toxic subunit of a toxin protein (ab2), a mutated subunit of a toxin protein having reduced or no toxicity (ab3), a mutated A-subunit of a toxin protein having reduced or no toxicity (ab4), a mutated A+B-subunit of a toxin protein having reduced or no toxicity (ab5), a mutated ricin holo-toxin having reduced or no toxicity (ab6), a non-toxic subunit of a ricin toxin protein (ab7), a mutated subunit of a ricin toxin protein having reduced or no toxicity (ab8), a mutated A-subunit of a ricin toxin protein having reduced or no toxicity (ab9), an A-subunit of a ricin toxin protein that comprises an R180H mutation (SEQ ID NO: 1) (ab10), a mutated A+B-subunit of a ricin toxin protein having reduced or no toxicity (ab11), a mutated Shiga holo-toxin having reduced or no toxicity (ab12), a non-toxic subunit of a Shiga toxin protein (ab13), a mutated subunit of a Shiga toxin protein having reduced or no toxicity (ab14), a mutated A-subunit of a Shiga toxin protein having reduced or no toxicity (ab15), a mutated A+B-subunit of a Shiga toxin protein having reduced or no toxicity (ab16), a mutated Stx1a holo-toxin having reduced or no toxicity (ab17), a non-toxic subunit of an Stx1a Shiga toxin protein (ab18), a mutated subunit of an Stx1a Shiga toxin protein having reduced or no toxicity (ab19), a mutated A-subunit of an Stx1a Shiga toxin protein having reduced or no toxicity (ab20), a mutated A+B-subunit of an Stx1a Shiga toxin protein having reduced or no toxicity (ab21), a mutated Stx1b holo-toxin having reduced or no toxicity (ab22), a non-toxic subunit of an Stx1b Shiga toxin protein (ab23), a mutated subunit of an Stx1b Shiga toxin protein having reduced or no toxicity (ab24), a mutated A-subunit of an Stx1b Shiga toxin protein having reduced or no toxicity (ab25), a mutated A+B-subunit of an Stx1b Shiga toxin protein having reduced or no toxicity (ab26), a mutated Stx1c holo-toxin having reduced or no toxicity (ab27), a non-toxic subunit of an Stx1c Shiga toxin protein (ab28), a mutated subunit of an Stx1c Shiga toxin protein having reduced or no toxicity (ab29), a mutated A-subunit of an Stx1c Shiga toxin protein having reduced or no toxicity (ab30), a mutated A+B-subunit of an Stx1c Shiga toxin protein having reduced or no toxicity (ab31), a mutated Stx1d holo-toxin having reduced or no toxicity (ab32), a non-toxic subunit of an Stx1d Shiga toxin protein (ab33), a mutated subunit of an Stx1d Shiga toxin protein having reduced or no toxicity (ab34), a mutated A-subunit of an Stx1d Shiga toxin protein having reduced or no toxicity (ab35), a mutated A+B-subunit of an Stx1d Shiga toxin protein having reduced or no toxicity (ab36), a mutated Stx2a holo-toxin having reduced or no toxicity (ab37), a non-toxic subunit of an Stx2a Shiga toxin protein (ab38), a mutated subunit of an Stx2a Shiga toxin protein having reduced or no toxicity (ab39), a mutated A-subunit of an Stx2a Shiga toxin protein having reduced or no toxicity (ab40), a mutated A+B-subunit of an Stx2a Shiga toxin protein having reduced or no toxicity (ab41), a mutated Stx2b holo-toxin having reduced or no toxicity (ab42), a non-toxic subunit of an Stx2b Shiga toxin protein (ab43), a mutated subunit of an Stx2b Shiga toxin protein having reduced or no toxicity (ab44), a mutated A-subunit of an Stx2b Shiga toxin protein having reduced or no toxicity (ab45), a mutated A+B-subunit of an Stx2b Shiga toxin protein having reduced or no toxicity (ab46), a mutated Stx2c holo-toxin having reduced or no toxicity (ab47), a non-toxic subunit of an Stx2c Shiga toxin protein (ab48), a mutated subunit of an Stx2c Shiga toxin protein having reduced or no toxicity (ab49), a mutated A-subunit of an Stx2c Shiga toxin protein having reduced or no toxicity (ab50), a mutated A+B-subunit of an Stx2c Shiga toxin protein having reduced or no toxicity (ab51), a mutated Stx2d holo-toxin having reduced or no toxicity (ab52), a non-toxic subunit of an Stx2d Shiga toxin protein (ab53), a mutated subunit of an Stx2d Shiga toxin protein having reduced or no toxicity (ab54), a mutated A-subunit of an Stx2d Shiga toxin protein having reduced or no toxicity (ab55), a mutated A+B-subunit of an Stx2d Shiga toxin protein having reduced or no toxicity (ab56), a mutated Stx2e holo-toxin having reduced or no toxicity (ab57), a non-toxic subunit of an Stx2e Shiga toxin protein (ab58), a mutated subunit of an Stx2e Shiga toxin protein having reduced or no toxicity (ab59), a mutated A-subunit of an Stx2e Shiga toxin protein having reduced or no toxicity (ab60), a mutated A+B-subunit of an Stx2e Shiga toxin protein having reduced or no toxicity (ab61), a mutated Stx2f holo-toxin having reduced or no toxicity (ab62), a non-toxic subunit of an Stx2f Shiga toxin protein (ab63), a mutated subunit of an Stx2f Shiga toxin protein having reduced or no toxicity (ab64), a mutated A-subunit of an Stx2f Shiga toxin protein having reduced or no toxicity (ab65), a mutated A+B-subunit of an Stx2f Shiga toxin protein having reduced or no toxicity (ab66), a mutated Stx2g holo-toxin having reduced or no toxicity (ab67), a non-toxic subunit of an Stx2g Shiga toxin protein (ab68), a mutated subunit of an Stx2g Shiga toxin protein having reduced or no toxicity (ab69), a mutated A-subunit of an Stx2g Shiga toxin protein having reduced or no toxicity (ab70), a mutated A+B-subunit of an Stx2g Shiga toxin protein having reduced or no toxicity (ab71), a mutated cholera holo-toxin having reduced or no toxicity (ab72), a non-toxic subunit of a cholera toxin protein (ab73), a mutated subunit of a cholera toxin protein having reduced or no toxicity (ab74), a mutated A-subunit of a cholera toxin protein having reduced or no toxicity (ab75), or an AMF (ab76).

Thus, a preferred conjugate of the present invention comprises, essentially consists of, or consists of or contains:
(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
   wherein the at least one module (a) and the at least one module (b) are comprised or contained within a [module (a)+module (b)] protein or peptide, wherein the [module (a)+module (b)] protein or peptide is selected from the group consisting of ab1, ab2, ab3, ab4, ab5, ab6, ab7, ab8, ab9, ab10, ab11, ab12, ab13, ab14, ab15, ab16, ab17, ab18, ab19, ab20, ab21, ab22, ab23, ab24, ab25, ab26, ab27, ab28, ab29, ab30, ab31, ab32, ab33, ab34, ab35, ab36, ab37, ab38, ab39, ab40, ab41, ab42, ab43, ab44, ab45, ab46, ab47, ab48, ab49, ab50, ab51, ab52, ab53, ab54, ab55, ab56, ab57, ab58, ab59, ab60, ab61, ab62, ab63, ab64, ab65, ab66, ab67, ab68, ab69, ab70, ab71, ab72, ab73, ab74, ab75, and ab76, and wherein the [module (a)+module (b)] protein or peptide, the at least one module (c), and the at least one compound (d) are linked to each other in any arrangement. The conjugates of the present invention optionally comprise a nuclear localization signal.
   (a+b) Peptide with Specific (c):

Another preferred conjugate of the present invention comprises, consists essentially of, consists of or contains:
(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
   wherein the at least one module (a) and the at least one module (b) are comprised or contained within a [module (a)+module (b)] protein or peptide, wherein the [module (a)+module (b)] protein or peptide and the at least one module (c) are selected from the group of combinations consisting of ab1+c1 (K1751), ab1+c2 (K1752), ab1+c3 (K1753), ab1+c4 (K1754), ab1+c5 (K1755), ab1+c6 (K1756), ab1+c7 (K1757), ab1+c8 (K1758), ab1+c9 (K1759), ab1+c10 (K1760), ab1+c11 (K1761), ab1+c12 (K1762), ab1+c13 (K1763), ab1+c14 (K1764), ab1+c15 (K1765), ab1+c16 (K1766), ab1+c17 (K1767), ab1+c18 (K1768), ab1+c19 (K1769), ab1+c20 (K1770), ab1+c21 (K1771), ab1+c22 (K1772), ab1+c23 (K1773), ab1+c24 (K1774), ab1+c25 (K1775), ab1+c26 (K1776), ab1+c27 (K1777), ab1+c28 (K1778), ab1+c29 (K1779), ab1+c30 (K1780), ab1+c31 (K1781), ab1+c32 (K1782), ab1+c33 (K1783), ab1+c34 (K1784), ab1+c35 (K1785), ab1+c36 (K1786), ab1+c37 (K1787), ab1+c38 (K1788), ab1+c39 (K1789), ab1+c40 (K1790), ab1+c41 (K1791), ab1+c42 (K1792), ab1+c43 (K1793), ab1+c44 (K1794), ab1+c45 (K1795), ab1+c46 (K1796), ab1+c47

(K1797), ab1+c48 (K1798), ab1+c49 (K1799), ab1+c50 (K1800), ab1+c51 (K1801), ab1+c52 (K1802), ab1+c53 (K1803), ab1+c54 (K1804), ab1+c55 (K1805), ab1+c56 (K1806), ab1+c57 (K1807), ab1+c58 (K1808), ab1+c59 (K1809), ab1+c60 (K1810), ab1+c61 (K1811), ab1+c62 (K1812), ab1+c63 (K1813), ab1+c64 (K1814), ab1+c65 (K1815), ab1+c66 (K1816), ab1+c67 (K1817), ab1+c68 (K1818), ab1+c69 (K1819), ab1+c70 (K1820), ab1+c71 (K1821), ab1+c72 (K1822), ab1+c73 (K1823), ab1+c74 (K1824), ab1+c75 (K1825), ab1+c76 (K1826), ab1+c77 (K1827), ab1+c78 (K1828), ab1+c79 (K1829), ab1+c80 (K1830), ab1+c81 (K1831), ab1+c82 (K1832), ab1+c83 (K1833), ab1+c84 (K1834), ab1+c85 (K1835), ab1+c86 (K1836), ab1+c87 (K1837), ab1+c88 (K1838), ab1+c89 (K1839), ab1+c90 (K1840), ab1+c91 (K1841), ab1+c92 (K1842), ab1+c93 (K1843), ab1+c94 (K1844), ab1+c95 (K1845), ab1+c96 (K1846), ab1+c97 (K1847), ab1+c98 (K1848), ab1+c99 (K1849), ab1+c100 (K1850), ab1+c101 (K1851), ab1+c102 (K1852), ab1+c103 (K1853), ab1+c104 (K1854), ab1+c105 (K1855), ab1+c106 (K1856), ab1+c107 (K1857), ab1+c108 (K1858), ab1+c109 (K1859), ab1+c110 (K1860), ab1+c111 (K1861), ab1+c112 (K1862), ab1+c113 (K1863), ab1+c114 (K1864), ab1+c115 (K1865), ab1+c116 (K1866), ab1+c117 (K1867), ab1+c118 (K1868), ab1+c119 (K1869), ab1+c120 (K1870), ab1+c121 (K1871), ab1+c122 (K1872), ab1+c123 (K1873), ab1+c124 (K1874), ab1+c125 (K1875), ab1+c126 (K1876), ab1+c127 (K1877), ab1+c128 (K1878), ab1+c129 (K1879), ab1+c130 (K1880), ab1+c131 (K1881), ab1+c132 (K1882), ab1+c133 (K1883), ab1+c134 (K1884), ab1+c135 (K1885), ab1+c136 (K1886), ab1+c137 (K1887), ab1+c138 (K1888), ab1+c139 (K1889), ab1+c140 (K1890), ab1+c141 (K1891), ab1+c142 (K1892), ab1+c143 (K1893), ab1+c144 (K1894), ab1+c145 (K1895), ab1+c146 (K1896), ab1+c147 (K1897), ab1+c148 (K1898), ab1+c149 (K1899), ab1+c150 (K1900), ab1+c151 (K1901), ab1+c152 (K1902), ab1+c153 (K1903), ab1+c154 (K1904), ab1+c155 (K1905), ab1+c156 (K1906), ab1+c157 (K1907), ab1+c158 (K1908), ab1+c159 (K1909), ab1+c160 (K1910), ab1+c161 (K1911), ab1+c162 (K1912), ab1+c163 (K1913), ab2+c1 (K1914), ab2+c2 (K1915), ab2+c3 (K1916), ab2+c4 (K1917), ab2+c5 (K1918), ab2+c6 (K1919), ab2+c7 (K1920), ab2+c8 (K1921), ab2+c9 (K1922), ab2+c10 (K1923), ab2+c11 (K1924), ab2+c12 (K1925), ab2+c13 (K1926), ab2+c14 (K1927), ab2+c15 (K1928), ab2+c16 (K1929), ab2+c17 (K1930), ab2+c18 (K1931), ab2+c19 (K1932), ab2+c20 (K1933), ab2+c21 (K1934), ab2+c22 (K1935), ab2+c23 (K1936), ab2+c24 (K1937), ab2+c25 (K1938), ab2+c26 (K1939), ab2+c27 (K1940), ab2+c28 (K1941), ab2+c29 (K1942), ab2+c30 (K1943), ab2+c31 (K1944), ab2+c32 (K1945), ab2+c33 (K1946), ab2+c34 (K1947), ab2+c35 (K1948), ab2+c36 (K1949), ab2+c37 (K1950), ab2+c38 (K1951), ab2+c39 (K1952), ab2+c40 (K1953), ab2+c41 (K1954), ab2+c42 (K1955), ab2+c43 (K1956), ab2+c44 (K1957), ab2+c45 (K1958), ab2+c46 (K1959), ab2+c47 (K1960), ab2+c48 (K1961), ab2+c49 (K1962), ab2+c50 (K1963), ab2+c51 (K1964), ab2+c52 (K1965), ab2+c53 (K1966), ab2+c54 (K1967), ab2+c55 (K1968), ab2+c56 (K1969), ab2+c57 (K1970), ab2+c58 (K1971), ab2+c59 (K1972), ab2+c60 (K1973), ab2+c61 (K1974), ab2+c62 (K1975), ab2+c63 (K1976), ab2+c64 (K1977), ab2+c65 (K1978), ab2+c66 (K1979), ab2+c67 (K1980), ab2+c68 (K1981), ab2+c69 (K1982), ab2+c70 (K1983), ab2+c71 (K1984), ab2+c72 (K1985), ab2+c73 (K1986), ab2+c74 (K1987), ab2+c75 (K1988), ab2+c76 (K1989), ab2+c77 (K1990), ab2+c78 (K1991), ab2+c79 (K1992), ab2+c80 (K1993), ab2+c81 (K1994), ab2+c82 (K1995), ab2+c83 (K1996), ab2+c84 (K1997), ab2+c85 (K1998), ab2+c86 (K1999), ab2+c87 (K2000), ab2+c88 (K2001), ab2+c89 (K2002), ab2+c90 (K2003), ab2+c91 (K2004), ab2+c92 (K2005), ab2+c93 (K2006), ab2+c94 (K2007), ab2+c95 (K2008), ab2+c96 (K2009), ab2+c97 (K2010), ab2+c98 (K2011), ab2+c99 (K2012), ab2+c100 (K2013), ab2+c101 (K2014), ab2+c102 (K2015), ab2+c103 (K2016), ab2+c104 (K2017), ab2+c105 (K2018), ab2+c106 (K2019), ab2+c107 (K2020), ab2+c108 (K2021), ab2+c109 (K2022), ab2+c110 (K2023), ab2+c111 (K2024), ab2+c112 (K2025), ab2+c113 (K2026), ab2+c114 (K2027), ab2+c115 (K2028), ab2+c116 (K2029), ab2+c117 (K2030), ab2+c118 (K2031), ab2+c119 (K2032), ab2+c120 (K2033), ab2+c121 (K2034), ab2+c122 (K2035), ab2+c123 (K2036), ab2+c124 (K2037), ab2+c125 (K2038), ab2+c126 (K2039), ab2+c127 (K2040), ab2+c128 (K2041), ab2+c129 (K2042), ab2+c130 (K2043), ab2+c131 (K2044), ab2+c132 (K2045), ab2+c133 (K2046), ab2+c134 (K2047), ab2+c135 (K2048), ab2+c136 (K2049), ab2+c137 (K2050), ab2+c138 (K2051), ab2+c139 (K2052), ab2+c140 (K2053), ab2+c141 (K2054), ab2+c142 (K2055), ab2+c143 (K2056), ab2+c144 (K2057), ab2+c145 (K2058), ab2+c146 (K2059), ab2+c147 (K2060), ab2+c148 (K2061), ab2+c149 (K2062), ab2+c150 (K2063), ab2+c151 (K2064), ab2+c152 (K2065), ab2+c153 (K2066), ab2+c154 (K2067), ab2+c155 (K2068), ab2+c156 (K2069), ab2+c157 (K2070), ab2+c158 (K2071), ab2+c159 (K2072), ab2+c160 (K2073), ab2+c161 (K2074), ab2+c162 (K2075), ab2+c163 (K2076), ab3+c1 (K2077), ab3+c2 (K2078), ab3+c3 (K2079), ab3+c4 (K2080), ab3+c5 (K2081), ab3+c6 (K2082), ab3+c7 (K2083), ab3+c8 (K2084), ab3+c9 (K2085), ab3+c10 (K2086), ab3+c11 (K2087), ab3+c12 (K2088), ab3+c13 (K2089), ab3+c14 (K2090), ab3+c15 (K2091), ab3+c16 (K2092), ab3+c17 (K2093), ab3+c18 (K2094), ab3+c19 (K2095), ab3+c20 (K2096), ab3+c21 (K2097), ab3+c22 (K2098), ab3+c23 (K2099), ab3+c24 (K2100), ab3+c25 (K2101), ab3+c26 (K2102), ab3+c27 (K2103), ab3+c28 (K2104), ab3+c29 (K2105), ab3+c30 (K2106), ab3+c31 (K2107), ab3+c32 (K2108), ab3+c33 (K2109), ab3+c34 (K2110), ab3+c35 (K2111), ab3+c36 (K2112), ab3+c37 (K2113), ab3+c38 (K2114), ab3+c39 (K2115), ab3+c40 (K2116), ab3+c41 (K2117), ab3+c42 (K2118), ab3+c43 (K2119), ab3+c44 (K2120), ab3+c45 (K2121), ab3+c46 (K2122), ab3+c47 (K2123), ab3+c48 (K2124), ab3+c49 (K2125), ab3+c50 (K2126), ab3+c51 (K2127), ab3+c52 (K2128), ab3+c53 (K2129), ab3+c54 (K2130), ab3+c55 (K2131), ab3+c56 (K2132), ab3+c57 (K2133), ab3+c58 (K2134), ab3+c59 (K2135), ab3+c60 (K2136), ab3+c61 (K2137), ab3+c62 (K2138), ab3+c63 (K2139), ab3+c64 (K2140), ab3+c65 (K2141), ab3+c66 (K2142), ab3+c67 (K2143), ab3+c68 (K2144), ab3+c69 (K2145), ab3+c70 (K2146), ab3+c71 (K2147), ab3+c72 (K2148), ab3+c73 (K2149), ab3+c74 (K2150), ab3+c75 (K2151), ab3+c76 (K2152), ab3+c77 (K2153), ab3+c78 (K2154), ab3+c79 (K2155), ab3+c80 (K2156), ab3+c81 (K2157), ab3+c82 (K2158), ab3+c83 (K2159), ab3+c84 (K2160), ab3+c85 (K2161), ab3+c86 (K2162), ab3+c87 (K2163), ab3+c88 (K2164), ab3+c89 (K2165), ab3+c90 (K2166), ab3+c91 (K2167), ab3+c92 (K2168), ab3+c93 (K2169), ab3+c94 (K2170), ab3+c95 (K2171), ab3+c96 (K2172), ab3+c97 (K2173), ab3+c98 (K2174), ab3+c99 (K2175), ab3+c100 (K2176), ab3+c101 (K2177), ab3+c102 (K2178), ab3+c103 (K2179), ab3+c104 (K2180), ab3+c105 (K2181), ab3+c106 (K2182), ab3+c107 (K2183), ab3+c108 (K2184), ab3+c109 (K2185), ab3+c110 (K2186), ab3+c111 (K2187), ab3+c112 (K2188), ab3+c113 (K2189), ab3+c114 (K2190), ab3+c115 (K2191), ab3+c116 (K2192), ab3+c117 (K2193), ab3+c118 (K2194), ab3+c119 (K2195), ab3+c120 (K2196), ab3+c121 (K2197), ab3+c122 (K2198), ab3+c123 (K2199), ab3+c124 (K2200), ab3+c125 (K2201), ab3+c126 (K2202), ab3+c127 (K2203), ab3+c128 (K2204), ab3+c129 (K2205), ab3+c130 (K2206), ab3+c131 (K2207), ab3+c132 (K2208), ab3+c133 (K2209), ab3+c134 (K2210), ab3+c135 (K2211), ab3+c136 (K2212), ab3+c137 (K2213), ab3+c138 (K2214), ab3+c139 (K2215), ab3+c140 (K2216), ab3+c141 (K2217), ab3+c142 (K2218), ab3+c143 (K2219), ab3+c144 (K2220), ab3+c145 (K2221), ab3+c146 (K2222), ab3+c147 (K2223), ab3+c148 (K2224), ab3+c149 (K2225), ab3+c150 (K2226), ab3+c151 (K2227), ab3+c152 (K2228), ab3+c153 (K2229), ab3+c154 (K2230), ab3+c155 (K2231), ab3+c156 (K2232), ab3+c157 (K2233), ab3+c158 (K2234), ab3+c159 (K2235), ab3+c160 (K2236), ab3+c161 (K2237), ab3+c162 (K2238), ab3+c163 (K2239), ab4+c1 (K2240), ab4+c2 (K2241), ab4+c3 (K2242), ab4+c4 (K2243), ab4+c5 (K2244), ab4+c6 (K2245), ab4+c7 (K2246), ab4+c8 (K2247), ab4+c9 (K2248), ab4+c10 (K2249), ab4+c11 (K2250), ab4+c12 (K2251), ab4+c13 (K2252), ab4+c14 (K2253), ab4+c15 (K2254), ab4+c16 (K2255), ab4+c17 (K2256), ab4+c18 (K2257), ab4+c19 (K2258), ab4+c20 (K2259), ab4+c21 (K2260), ab4+c22 (K2261), ab4+c23 (K2262), ab4+c24 (K2263), ab4+c25 (K2264), ab4+c26 (K2265), ab4+c27 (K2266), ab4+c28 (K2267), ab4+c29 (K2268), ab4+c30 (K2269), ab4+c31 (K2270), ab4+c32 (K2271), ab4+c33 (K2272), ab4+c34 (K2273), ab4+c35 (K2274), ab4+c36 (K2275), ab4+c37 (K2276), ab4+c38 (K2277), ab4+c39 (K2278), ab4+c40 (K2279), ab4+c41 (K2280), ab4+c42 (K2281), ab4+c43 (K2282), ab4+c44 (K2283), ab4+c45 (K2284), ab4+c46 (K2285), ab4+c47 (K2286), ab4+c48 (K2287), ab4+c49 (K2288), ab4+c50 (K2289), ab4+c51 (K2290), ab4+c52 (K2291), ab4+c53 (K2292), ab4+c54 (K2293), ab4+c55 (K2294), ab4+c56 (K2295), ab4+c57 (K2296), ab4+c58 (K2297), ab4+c59 (K2298), ab4+c60 (K2299), ab4+c61 (K2300), ab4+c62 (K2301), ab4+c63 (K2302), ab4+c64 (K2303), ab4+c65 (K2304), ab4+c66 (K2305), ab4+c67 (K2306), ab4+c68 (K2307), ab4+c69 (K2308), ab4+c70 (K2309), ab4+c71 (K2310), ab4+c72 (K2311), ab4+c73 (K2312), ab4+c74 (K2313), ab4+c75 (K2314), ab4+c76 (K2315), ab4+c77 (K2316), ab4+c78 (K2317), ab4+c79 (K2318), ab4+c80 (K2319), ab4+c81 (K2320), ab4+c82 (K2321), ab4+c83 (K2322), ab4+c84 (K2323), ab4+c85 (K2324), ab4+c86 (K2325), ab4+c87 (K2326), ab4+c88 (K2327), ab4+c89 (K2328), ab4+c90 (K2329), ab4+c91 (K2330), ab4+c92 (K2331), ab4+c93 (K2332), ab4+c94 (K2333), ab4+c95 (K2334), ab4+c96 (K2335), ab4+c97 (K2336), ab4+c98 (K2337), ab4+c99 (K2338), ab4+c100 (K2339), ab4+c101 (K2340), ab4+c102 (K2341), ab4+c103 (K2342), ab4+c104 (K2343), ab4+c105 (K2344), ab4+c106 (K2345), ab4+c107 (K2346), ab4+c108 (K2347), ab4+c109 (K2348), ab4+c110 (K2349), ab4+c111 (K2350), ab4+c112 (K2351), ab4+c113 (K2352), ab4+c114 (K2353), ab4+c115 (K2354), ab4+c116 (K2355), ab4+c117 (K2356), ab4+c118 (K2357), ab4+c119 (K2358), ab4+c120 (K2359), ab4+c121 (K2360), ab4+c122 (K2361), ab4+c123 (K2362), ab4+c124 (K2363), ab4+c125 (K2364), ab4+c126 (K2365), ab4+c127 (K2366), ab4+c128 (K2367), ab4+c129 (K2368), ab4+c130 (K2369), ab4+c131 (K2370), ab4+c132 (K2371), ab4+c133 (K2372), ab4+c134 (K2373), ab4+c135 (K2374), ab4+c136 (K2375), ab4+c137 (K2376), ab4+c138 (K2377), ab4+c139 (K2378), ab4+c140 (K2379), ab4+c141 (K2380), ab4+c142 (K2381), ab4+c143 (K2382), ab4+c144 (K2383), ab4+c145 (K2384), ab4+c146 (K2385), ab4+c147 (K2386), ab4+c148 (K2387), ab4+c149 (K2388), ab4+c150 (K2389), ab4+c151 (K2390), ab4+c152 (K2391), ab4+c153 (K2392), ab4+c154 (K2393), ab4+c155 (K2394), ab4+c156 (K2395), ab4+c157 (K2396), ab4+c158 (K2397), ab4+c159 (K2398), ab4+c160 (K2399), ab4+c161 (K2400), ab4+c162 (K2401), ab4+c163 (K2402), ab5+c1 (K2403), ab5+c2 (K2404), ab5+c3 (K2405), ab5+c4 (K2406), ab5+c5 (K2407), ab5+c6 (K2408), ab5+c7 (K2409), ab5+c8 (K2410), ab5+c9 (K2411), ab5+c10 (K2412), ab5+c11 (K2413), ab5+c12 (K2414), ab5+c13 (K2415), ab5+c14 (K2316), ab5+c15 (K2417), ab5+c16 (K2418), ab5+c17 (K2419), ab5+c18 (K2420), ab5+c19 (K2421), ab5+c20 (K2422), ab5+c21 (K2423), ab5+c22 (K2424), ab5+c23 (K2425), ab5+c24 (K2426), ab5+c25 (K2427), ab5+c26 (K2428), ab5+c27 (K2429), ab5+c28 (K2430), ab5+c29 (K2431), ab5+c30 (K2432), ab5+c31 (K2433), ab5+c32 (K2434), ab5+c33 (K2435), ab5+c34 (K2436), ab5+c35 (K2437), ab5+c36 (K2438), ab5+c37 (K2439), ab5+c38 (K2440), ab5+c39 (K2441), ab5+c40 (K2442), ab5+c41 (K2443), ab5+c42 (K2444), ab5+c43 (K2445), ab5+c44 (K2446), ab5+c45 (K2447), ab5+c46 (K2448), ab5+c47 (K2449), ab5+c48 (K2450), ab5+c49 (K2451), ab5+c50 (K2452), ab5+c51 (K2453), ab5+c52 (K2454), ab5+c53 (K2455), ab5+c54 (K2456), ab5+c55 (K2457), ab5+c56 (K2458), ab5+c57 (K2459), ab5+c58 (K2460), ab5+c59 (K2461), ab5+c60 (K2462), ab5+c61 (K2463), ab5+c62 (K2464), ab5+c63 (K2465), ab5+c64 (K2466), ab5+c65 (K2467), ab5+c66 (K2468), ab5+c67 (K2469), ab5+c68 (K2470), ab5+c69 (K2471), ab5+c70 (K2472), ab5+c71 (K2473), ab5+c72 (K2474), ab5+c73 (K2475), ab5+c74 (K2476), ab5+c75 (K2477), ab5+c76 (K2478), ab5+c77 (K2479), ab5+c78 (K2480), ab5+c79 (K2481), ab5+c80 (K2482), ab5+c81 (K2483), ab5+c82 (K2484), ab5+c83 (K2485), ab5+c84 (K2486), ab5+c85 (K2487), ab5+c86 (K2488), ab5+c87 (K2489), ab5+c88 (K2490), ab5+c89 (K2491), ab5+c90 (K2492), ab5+c91 (K2493), ab5+c92 (K2494), ab5+c93 (K2495), ab5+c94 (K2496), ab5+c95 (K2497), ab5+c96 (K2498), ab5+c97 (K2499), ab5+c98 (K2500), ab5+c99 (K2501), ab5+c100 (K2502), ab5+c101 (K2503), ab5+c102 (K2504), ab5+c103 (K2505), ab5+c104 (K2506), ab5+c105 (K2507), ab5+c106 (K2508), ab5+c107 (K2509), ab5+c108 (K2510), ab5+c109 (K2511), ab5+c110 (K2512), ab5+c111 (K2513), ab5+c112 (K2514), ab5+c113 (K2515), ab5+c114 (K2516), ab5+c115 (K2517), ab5+c116 (K2518), ab5+c117 (K2519), ab5+c118 (K2520), ab5+c119 (K2521), ab5+c120 (K2522), ab5+c121 (K2523), ab5+c122 (K2524), ab5+c123 (K2525), ab5+c124 (K2526), ab5+c125 (K2527), ab5+c126 (K2528), ab5+c127 (K2529), ab5+c128 (K2530), ab5+c129 (K2531), ab5+c130 (K2532), ab5+c131 (K2533), ab5+c132 (K2534), ab5+c133 (K2535), ab5+c134 (K2536), ab5+c135 (K2537), ab5+c136 (K2538), ab5+c137 (K2539), ab5+c138 (K2540), ab5+c139 (K2541), ab5+c140 (K2542), ab5+c141 (K2543), ab5+c142 (K2544), ab5+c143 (K2545), ab5+c144 (K2546), ab5+c145 (K2547), ab5+c146 (K2548), ab5+c147 (K2549), ab5+c148 (K2550), ab5+c149 (K2551), ab5+c150 (K2552), ab5+c151 (K2553), ab5+c152 (K2554), ab5+c153 (K2555), ab5+c154 (K2556), ab5+c155 (K2557), ab5+c156 (K2558), ab5+c157 (K2559), ab5+c158 (K2560), ab5+c159 (K2561), ab5+c160 (K2562), ab5+c161 (K2563), ab5+c162 (K2564), ab5+c163 (K2565), ab6+c1 (K2566), ab6+c2 (K2567), ab6+c3 (K2568), ab6+c4 (K2569), ab6+c5 (K2570), ab6+c6 (K2571), ab6+c7 (K2572), ab6+c8 (K2573), ab6+c9 (K2574), ab6+c10 (K2575), ab6+c11 (K2576), ab6+c12 (K2577), ab6+c13 (K2578), ab6+c14 (K2579), ab6+c15 (K2580), ab6+c16 (K2581), ab6+c17 (K2582), ab6+c18 (K2583), ab6+c19 (K2584), ab6+c20 (K2585), ab6+c21 (K2586), ab6+c22 (K2587), ab6+c23 (K2588), ab6+c24 (K2589), ab6+c25 (K2590), ab6+c26 (K2591), ab6+c27 (K2592), ab6+c28 (K2593), ab6+c29 (K2594), ab6+c30 (K2595), ab6+c31 (K2596), ab6+c32 (K2597), ab6+c33 (K2598), ab6+c34 (K2599), ab6+c35 (K2600), ab6+c36 (K2601), ab6+c37 (K2602), ab6+c38 (K2603), ab6+c39 (K2604), ab6+c40 (K2605), ab6+c41 (K2606), ab6+c42 (K2607), ab6+c43 (K2608), ab6+c44 (K2609), ab6+c45 (K2610), ab6+c46 (K2611), ab6+c47 (K2612), ab6+c48 (K2613), ab6+c49 (K2614), ab6+c50 (K2615), ab6+c51 (K2616), ab6+c52 (K2617), ab6+c53 (K2618), ab6+c54 (K2619), ab6+c55 (K2620), ab6+c56 (K2621), ab6+c57 (K2622), ab6+c58 (K2623), ab6+c59 (K2624), ab6+c60 (K2625), ab6+c61 (K2626), ab6+c62 (K2627), ab6+c63 (K2628), ab6+c64 (K2629), ab6+c65 (K2630), ab6+c66 (K2631), ab6+c67 (K2632), ab6+c68 (K2633), ab6+c69 (K2634), ab6+c70 (K2635), ab6+c71 (K2636), ab6+c72 (K2637), ab6+c73 (K2638), ab6+c74 (K2639), ab6+c75 (K2640), ab6+c76 (K2641), ab6+c77 (K2642), ab6+c78 (K2643), ab6+c79 (K2644), ab6+c80 (K2645), ab6+c81 (K2646), ab6+c82 (K2647), ab6+c83 (K2648), ab6+c84 (K2649), ab6+c85 (K2650), ab6+c86 (K2651), ab6+c87 (K2652), ab6+c88 (K2653), ab6+c89 (K2654), ab6+c90 (K2655), ab6+c91 (K2656), ab6+c92 (K2657), ab6+c93 (K2658), ab6+c94 (K2659), ab6+c95 (K2660), ab6+c96 (K2661), ab6+c97 (K2662), ab6+c98 (K2663), ab6+c99 (K2664), ab6+c100 (K2665), ab6+c101 (K2666), ab6+c102 (K2667), ab6+c103 (K2668), ab6+c104 (K2669), ab6+c105 (2670), ab6+c106 (K2671), ab6+c107 (K2672), ab6+c108 (K2673), ab6+c109 (K2674), ab6+c110 (K2675), ab6+c111 (K2676), ab6+c112 (K2677), ab6+c113 (K2678), ab6+c114 (K2679), ab6+c115 (K2680), ab6+c116 (K2681), ab6+c117 (K2682), ab6+c118 (K2683), ab6+c119 (K2684), ab6+c120 (K2685), ab6+c121 (K2686), ab6+c122 (K2687), ab6+c123 (K2688), ab6+c124 (K2689), ab6+c125 (K2690), ab6+c126 (K2691), ab6+c127 (K2692), ab6+c128 (K2693), ab6+c129 (K2694), ab6+c130 (K2695), ab6+c131 (K2696), ab6+c132 (K2697), ab6+c133 (K2698), ab6+c134 (K2699), ab6+c135 (K2700), ab6+c136 (K2701), ab6+c137 (K2702), ab6+c138 (K2703), ab6+c139 (K2704), ab6+c140 (K2705), ab6+c141 (K2706), ab6+c142 (K2707), ab6+c143 (K2708), ab6+c144 (K2709), ab6+c145 (K2710), ab6+c146 (K2711), ab6+c147 (K2712), ab6+c148 (K2713), ab6+c149 (K2714), ab6+c150 (K2715), ab6+c151 (K2716), ab6+c152 (K2717), ab6+c153 (K2718), ab6+c154 (K2719), ab6+c155 (K2720), ab6+c156 (K2721), ab6+c157 (K2722), ab6+c158 (K2723), ab6+c159 (K2724), ab6+c160 (K2725), ab6+c161 (K2726), ab6+c162 (K2727), ab6+c163 (K2728), ab7+c1 (K2729), ab7+c2 (K2730), ab7+c3 (K2731), ab7+c4 (K2732), ab7+c5 (K2733), ab7+c6 (K2734), ab7+c7 (K2735), ab7+c8 (K2736), ab7+c9 (K2737), ab7+c10 (K2738), ab7+c11 (K2739), ab7+c12 (K2740), ab7+c13 (K2741), ab7+c14 (K2742), ab7+c15 (K2743), ab7+c16 (K2744), ab7+c17 (K2745), ab7+c18 (K2746), ab7+c19 (K2747), ab7+c20 (K2748), ab7+c21 (K2749), ab7+c22 (K2750), ab7+c23 (K2751), ab7+c24 (K2752), ab7+c25 (K2753), ab7+c26 (K2754), ab7+c27 (K2755), ab7+c28 (K2756), ab7+c29 (K2757), ab7+c30 (K2758), ab7+c31 (K2759), ab7+c32 (K2760), ab7+c33 (K2761), ab7+c34 (K2762), ab7+c35 (K2763), ab7+c36 (K2764), ab7+c37 (K2765), ab7+c38 (K2766), ab7+c39 (K2767), ab7+c40 (K2768), ab7+c41 (K2769), ab7+c42 (K2770), ab7+c43 (K2771), ab7+c44 (K2772), ab7+c45 (K2773), ab7+c46 (K2774), ab7+c47 (K2775), ab7+c48 (K2776), ab7+c49 (K2777), ab7+c50 (K2778), ab7+c51 (K2779), ab7+c52 (K2780), ab7+c53 (K2781), ab7+c54 (K2782), ab7+c55 (K2783), ab7+c56 (K2784), ab7+c57 (K2785), ab7+c58 (K2786), ab7+c59 (K2787), ab7+c60 (K2788), ab7+c61 (K2789), ab7+c62 (K2790), ab7+c63 (K2791), ab7+c64 (K2792), ab7+c65 (K2793), ab7+c66 (K2794), ab7+c67 (K2795), ab7+c68 (K2796), ab7+c69 (K2797), ab7+c70 (K2798), ab7+c71 (K2799), ab7+c72 (K2800), ab7+c73 (K2801), ab7+c74 (K2802), ab7+c75 (K2803), ab7+c76 (K2804), ab7+c77 (K2805), ab7+c78 (K2806), ab7+c79 (2807), ab7+c80 (K2808), ab7+c81 (K2809), ab7+c82 (K2810), ab7+c83 (K2811), ab7+c84 (K2812), ab7+c85 (K2813), ab7+c86 (K2814), ab7+c87 (K2815), ab7+c88 (K2816), ab7+c89 (K2817), ab7+c90 (K2818), ab7+c91 (K2819), ab7+c92 (K2820), ab7+c93 (K2821), ab7+c94 (K2822), ab7+c95 (K2823), ab7+c96 (K2824), ab7+c97 (K2825), ab7+c98 (K2826), ab7+c99 (K2827), ab7+c100 (K2828), ab7+c101 (K2829), ab7+c102 (K2830), ab7+c103 (K2831), ab7+c104 (K2832), ab7+c105 (K2833), ab7+c106 (K2834), ab7+c107 (K2835), ab7+c108 (K2836), ab7+c109 (K2837), ab7+c110 (K2838), ab7+c111 (K2839), ab7+c112 (K2840), ab7+c113 (K2841), ab7+c114 (K2842), ab7+c115 (K2843), ab7+c116 (K2844), ab7+c117 (K2845), ab7+c118 (K2846), ab7+c119 (K2847), ab7+c120 (K2848), ab7+c121 (K2849), ab7+c122 (K2850), ab7+c123 (K2851), ab7+c124 (K2852), ab7+c125 (K2853), ab7+c126 (K2854), ab7+c127 (K2855), ab7+c128 (K2856), ab7+c129 (K2857), ab7+c130 (K2858), ab7+c131 (K2859), ab7+c132 (K2860), ab7+c133 (K2861), ab7+c134 (K2862), ab7+c135 (K2863), ab7+c136 (K2864), ab7+c137 (K2865), ab7+c138 (K2866), ab7+c139 (K2867), ab7+c140 (K2868), ab7+c141 (K2869), ab7+c142 (K2870), ab7+c143 (K2871), ab7+c144 (K2872), ab7+c145 (K2873), ab7+c146 (K2874), ab7+c147 (K2875), ab7+c148 (K2876), ab7+c149 (K2877), ab7+c150 (K2878), ab7+c151 (K2879), ab7+c152 (K2880), ab7+c153 (K2881), ab7+c154 (K2882), ab7+c155 (K2883), ab7+c156 (K2884), ab7+c157 (K2885), ab7+c158 (K2886), ab7+c159 (K2887), ab7+c160 (K2888), ab7+c161 (K2889), ab7+c162 (K2890), ab7+c163 (K2891), ab8+c1 (K2892), ab8+c2 (K2893), ab8+c3 (K2894), ab8+c4 (K2895), ab8+c5 (K2896), ab8+c6 (K2897), ab8+c7 (K2898), ab8+c8 (K2899), ab8+c9 (K2900), ab8+c10 (K2901), ab8+c11 (K2902), ab8+c12 (K2903), ab8+c13 (K2904), ab8+c14 (K2905), ab8+c15 (K2906), ab8+c16 (K2907), ab8+c17 (K2908), ab8+c18 (K2909), ab8+c19 (K2910), ab8+c20 (K2911), ab8+c21 (K2912), ab8+c22 (K2913), ab8+c23 (K2914), ab8+c24 (K2915), ab8+c25 (K2916), ab8+c26 (K2917), ab8+c27 (K2918), ab8+c28 (K2919), ab8+c29 (K2920), ab8+c30 (K2921), ab8+c31 (K2922), ab8+c32 (K2923), ab8+c33 (K2924), ab8+c34 (K2925), ab8+c35 (K2926), ab8+c36 (K2927), ab8+c37 (K2928), ab8+c38 (K2929), ab8+c39 (K2930), ab8+c40 (K2931), ab8+c41 (K2932), ab8+c42 (K2933), ab8+c43 (K2934), ab8+c44 (K2935), ab8+c45 (K2936), ab8+c46 (K2937), ab8+c47 (K2938), ab8+c48 (K2939), ab8+c49 (K2940), ab8+c50 (K2941), ab8+c51 (K2942), ab8+c52 (K2943), ab8+c53 (K2944), ab8+c54 (K2945), ab8+c55 (K2946), ab8+c56 (K2947), ab8+c57 (K2948), ab8+c58 (K2949), ab8+c59 (K2950), ab8+c60 (K2951), ab8+c61 (K2952), ab8+c62 (K2953), ab8+c63 (K2954), ab8+c64 (K2955), ab8+c65 (K2956), ab8+c66 (K2957), ab8+c67 (K2958), ab8+c68 (K2959), ab8+c69 (K2960), ab8+c70 (K2961), ab8+c71 (K2962), ab8+c72 (K2963), ab8+c73 (K2964), ab8+c74 (K2965), ab8+c75 (K2966), ab8+c76 (K2967), ab8+c77 (K2968), ab8+c78 (K2969), ab8+c79 (K2970), ab8+c80 (K2971), ab8+c81 (K2972), ab8+c82 (K2973), ab8+c83 (K2974), ab8+c84 (K2975), ab8+c85 (K2976), ab8+c86 (K2977), ab8+c87 (K2978), ab8+c88 (K2979), ab8+c89 (K2980), ab8+c90 (K2981), ab8+c91 (K2982), ab8+c92 (K2983), ab8+c93 (K2984), ab8+c94 (K2985), ab8+c95 (K2986), ab8+c96 (K2987), ab8+c97 (K2988), ab8+c98 (K2989), ab8+c99 (K2990), ab8+c100 (K2991), ab8+c101 (K2992), ab8+c102 (K2993), ab8+c103 (K2994), ab8+c104 (K2995), ab8+c105 (K2996), ab8+c106 (K2997), ab8+c107 (K2998), ab8+c108 (K2999), ab8+c109 (K3000), ab8+c110 (K3001), ab8+c111 (K3002), ab8+c112 (K3003), ab8+c113 (K3004), ab8+c114 (K3005), ab8+c115 (K3006), ab8+c116 (K3007), ab8+c117 (K3008), ab8+c118 (K3009), ab8+c119 (K3010), ab8+c120 (K3011), ab8+c121 (K3012), ab8+c122 (K3013), ab8+c123 (K3014), ab8+c124 (K3015), ab8+c125 (K3016), ab8+c126 (K3017), ab8+c127 (K3018), ab8+c128 (K3019), ab8+c129 (K3020), ab8+c130 (K3021), ab8+c131 (K3022), ab8+c132 (K3023), ab8+c133 (K3024), ab8+c134 (K3025), ab8+c135 (K3026), ab8+c136 (K3027), ab8+c137 (K3028), ab8+c138 (K3029), ab8+c139 (K3030), ab8+c140 (K3031), ab8+c141 (K3032), ab8+c142 (K3033), ab8+c143 (K3034), ab8+c144 (K3035), ab8+c145 (K3036), ab8+c146 (K3037), ab8+c147 (K3038), ab8+c148 (K3039), ab8+c149 (K3040), ab8+c150 (K3041), ab8+c151 (K3042), ab8+c152 (K3043), ab8+c153 (K3044), ab8+c154 (K3045), ab8+c155 (K3046), ab8+c156 (K3047), ab8+c157 (K3048), ab8+c158 (K3049), ab8+c159 (K3050), ab8+c160 (K3051), ab8+c161 (K3052), ab8+c162 (K3053), ab8+c163 (K3054), ab9+c1 (K3055), ab9+c2 (K3056), ab9+c3 (K3057), ab9+c4 (K3058), ab9+c5 (K3059), ab9+c6 (K3060), ab9+c7 (K3061), ab9+c8 (K3062), ab9+c9 (K3063), ab9+c10 (K3064), ab9+c11 (K3065), ab9+c12 (K3066), ab9+c13 (K3067), ab9+c14 (K3068), ab9+c15 (K3069), ab9+c16 (K3070), ab9+c17 (K3071), ab9+c18 (K3072), ab9+c19 (K3073), ab9+c20 (K3074), ab9+c21 (K3075), ab9+c22 (K3076), ab9+c23 (K3077), ab9+c24 (K3078), ab9+c25 (K3079), ab9+c26 (K3080), ab9+c27 (K3081), ab9+c28 (K3082), ab9+c29 (K3083), ab9+c30 (K3084), ab9+c31 (K3085), ab9+c32 (K3086), ab9+c33 (K3087), ab9+c34 (K3088), ab9+c35 (K3089), ab9+c36 (K3090), ab9+c37 (K3091), ab9+c38 (K3092), ab9+c39 (K3093), ab9+c40 (K3094), ab9+c41 (K3095), ab9+c42 (K3096), ab9+c43 (K3097), ab9+c44 (K3098), ab9+c45 (K3099), ab9+c46 (K3100), ab9+c47 (K3101), ab9+c48 (K3102), ab9+c49 (K3103), ab9+c50 (K3104), ab9+c51 (K3105), ab9+c52 (K3106), ab9+c53 (K3107), ab9+c54 (K3108), ab9+c55 (K3109), ab9+c56 (K3110), ab9+c57 (K3111), ab9+c58 (K3112), ab9+c59 (K3113), ab9+c60 (K3114), ab9+c61 (K3115), ab9+c62 (K3116), ab9+c63 (K3117), ab9+c64 (K3118), ab9+c65 (K3119), ab9+c66 (K3120), ab9+c67 (K3121), ab9+c68 (K3122), ab9+c69 (K3123), ab9+c70 (K3124), ab9+c71 (K3125), ab9+c72 (K3126), ab9+c73 (K3127), ab9+c74 (K3128), ab9+c75 (K3129), ab9+c76 (K3130), ab9+c77 (K3131), ab9+c78 (K3132), ab9+c79 (K3133), ab9+c80 (K3134), ab9+c81 (K3135), ab9+c82 (K3136), ab9+c83 (K3137), ab9+c84 (K3138), ab9+c85 (K3139), ab9+c86 (K3140), ab9+c87 (K3141), ab9+c88 (K3142), ab9+c89 (K3143), ab9+c90 (K3144), ab9+c91 (K3145), ab9+c92 (K3146), ab9+c93 (K3147), ab9+c94 (K3148), ab9+c95 (K3149), ab9+c96 (K3150), ab9+c97 (K3151), ab9+c98 (K3152), ab9+c99 (K3153), ab9+c100 (K3154), ab9+c101 (K3155), ab9+c102 (K3156), ab9+c103 (K3157), ab9+c104 (K3158), ab9+c105 (K3159), ab9+c106 (K3160), ab9+c107 (K3161), ab9+c108 (K3162), ab9+c109 (K3163), ab9+c110 (K3164), ab9+c111 (K3165), ab9+c112 (K3166), ab9+c113 (K3167), ab9+c114 (K3168), ab9+c115 (K3169), ab9+c116 (K3170), ab9+c117 (K3171), ab9+c118 (K3172), ab9+c119 (K3173), ab9+c120 (K3174), ab9+c121 (K3175), ab9+c122 (K3176), ab9+c123 (K3177), ab9+c124 (K3178), ab9+c125 (K3179), ab9+c126 (K3180), ab9+c127 (K3181), ab9+c128 (K3182), ab9+c129 (K3183), ab9+c130 (K3184), ab9+c131 (K3185), ab9+c132 (K3186), ab9+c133 (K3187), ab9+c134 (K3188), ab9+c135 (K3189), ab9+c136 (K3190), ab9+c137 (K3191), ab9+c138 (K3192), ab9+c139 (K3193), ab9+c140 (K3194), ab9+c141 (K3195), ab9+c142 (K3196), ab9+c143 (K3197), ab9+c144 (K3198), ab9+c145 (K3199), ab9+c146 (K3200), ab9+c147 (K3201), ab9+c148 (K3202), ab9+c149 (K3203), ab9+c150 (K3204), ab9+c151 (K3205), ab9+c152 (K3206), ab9+c153 (K3207), ab9+c154 (K3208), ab9+c155 (K3209), ab9+c156 (K3210), ab9+c157 (K3211), ab9+c158 (K3212), ab9+c159 (K3213), ab9+c160 (K3214), ab9+c161 (K3215), ab9+c162 (K3216), ab9+c163 (K3217), ab10+c1 (K3218), ab10+c2 (K3219), ab10+c3 (K3220), ab10+c4 (K3221), ab10+c5 (K3222), ab10+c6 (K3223), ab10+c7 (K3224), ab10+c8 (K3225), ab10+c9 (K3226), ab10+c10 (K3227), ab10+c11 (K3228), ab10+c12 (K3229), ab10+c13 (K3230), ab10+c14 (K3231), ab10+c15 (K3232), ab10+c16 (K3233), ab10+c17 (K3234), ab10+c18 (K3235), ab10+c19 (K3236), ab10+c20 (K3237), ab10+c21 (K3238), ab10+c22 (K3239), ab10+c23 (K3240), ab10+c24 (K3241), ab10+c25 (K3242), ab10+c26 (K3243), ab10+c27 (K3244), ab10+c28 (K3245), ab10+c29 (K3246), ab10+c30 (K3247), ab10+c31 (K3248), ab10+c32 (K3249), ab10+c33 (K3250), ab10+c34 (K3251), ab10+c35 (K3252), ab10+c36 (K3253), ab10+c37 (K3254), ab10+c38 (K3255), ab10+c39 (K3256), ab10+c40 (K3257), ab10+c41 (K3258), ab10+c42 (K3259), ab10+c43 (K3260), ab10+c44 (K3261), ab10+c45 (K3262), ab10+c46 (K3263), ab10+c47 (K3264), ab10+c48 (K3265), ab10+c49 (K3266), ab10+c50 (K3267), ab10+c51 (K3268), ab10+c52 (K3269), ab10+c53 (K3270), ab10+c54 (K3271), ab10+c55 (K3272), ab10+c56 (K3273), ab10+c57 (K3274), ab10+c58 (K3275), ab10+c59 (K3276), ab10+c60 (K3277), ab10+c61 (K3278), ab10+c62 (K3279), ab10+c63 (K3280), ab10+c64 (K3281), ab10+c65 (K3282), ab10+c66 (K3283), ab10+c67 (K3284), ab10+c68 (K3285), ab10+c69 (K3286), ab10+c70 (K3287), ab10+c71 (K3288), ab10+c72 (K3289), ab10+c73 (K3290), ab10+c74 (K3291), ab10+c75 (K3292), ab10+c76 (K3293), ab10+c77 (K3294), ab10+c78 (K3295), ab10+c79 (K3296), ab10+c80 (K3297), ab10+c81 (K3298), ab10+c82 (K3299), ab10+c83 (K3300), ab10+c84 (K3301), ab10+c85 (K3302), ab10+c86 (K3303), ab10+c87 (K3304), ab10+c88 (K3305), ab10+c89 (K3306), ab10+c90 (K3307), ab10+c91 (K3308), ab10+c92 (K3309), ab10+c93 (K3310), ab10+c94 (K3311), ab10+c95 (K3312), ab10+c96 (K3313), ab10+c97 (K3314), ab10+c98 (K3315), ab10+c99 (K3316), ab10+c100 (K3317), ab10+c101 (K3318), ab10+c102 (K3319), ab10+c103 (K3320), ab10+c104 (K3321), ab10+c105 (K3322), ab10+c106 (K3323), ab10+c107 (K3324), ab10+c108 (K3325), ab10+c109 (K3326), ab10+c110 (K3327), ab10+c111 (K3328), ab10+c112 (K3329), ab10+c113 (K3330), ab10+c114 (K3331), ab10+c115 (K3332), ab10+c116 (K3333), ab10+c117 (K3334), ab10+c118 (K3335), ab10+c119 (K3336), ab10+c120 (K3337), ab10+c121 (K3338), ab10+c122 (K3339), ab10+c123 (K3340), ab10+c124 (K3341), ab10+c125 (K3342), ab10+c126 (K3343), ab10+c127 (K3344), ab10+c128 (K3345), ab10+c129 (K3346), ab10+c130 (K3347), ab10+c131 (K3348), ab10+c132 (K3349), ab10+c133 (K3350), ab10+c134 (K3351), ab10+c135 (K3352), ab10+c136 (K3353), ab10+c137 (K3354), ab10+c138 (K3355), ab10+c139 (K3356), ab10+c140 (K3357), ab10+c141 (K3358), ab10+c142 (K3359), ab10+c143 (K3360), ab10+c144 (K3361), ab10+c145 (K3362), ab10+c146 (K3363), ab10+c147 (K3364), ab10+c148 (K3365), ab10+c149 (K3366), ab10+c150 (K3367), ab10+c151 (K3368), ab10+c152 (K3369), ab10+c153 (K3370), ab10+c154 (K3371), ab10+c155 (K3372), ab10+c156 (K3373), ab10+c157 (K3374), ab10+c158 (K3375), ab10+c159 (K3376), ab10+c160 (K3377), ab10+c161 (K3378), ab10+c162 (K3379), ab10+c163 (K3380), ab11+c1 (K3381), ab11+c2 (K3382), ab11+c3 (K3383), ab11+c4 (K3384), ab11+c5 (K3385), ab11+c6 (K3386), ab11+c7 (K3387), ab11+c8 (K3388), ab11+c9 (K3389), ab11+c10 (K3390), ab11+c11 (K3391), ab11+c12 (K3392), ab11+c13 (K3393), ab11+c14 (K3394), ab11+c15 (K3395), ab11+c16 (K3396), ab11+c17 (K3397), ab11+c18 (K3398), ab11+c19 (K3399), ab11+c20 (K3400), ab11+c21 (K3401), ab11+c22 (K3402), ab11+c23 (K3403), ab11+c24 (K3404), ab11+c25 (K3405), ab11+c26 (3406), ab11+c27 (K3407), ab11+c28 (3408), ab11+c29 (K3409), ab11+c30 (K3410), ab11+c31 (K3411), ab11+c32 (K3412), ab11+c33 (K3413), ab11+c34 (K3414), ab11+c35 (K3415), ab11+c36 (K3416), ab11+c37 (K3417), ab11+c38 (K3418), ab11+c39 (K3419), ab11+c40 (K3420), ab11+c41 (K3421), ab11+c42 (K3422), ab11+c43 (K3423), ab11+c44 (K3424), ab11+c45 (K3425), ab11+c46 (K3426), ab11+c47 (K3427), ab11+c48 (K3428), ab11+c49 (K3429), ab11+c50 (K3430), ab11+c51 (K3431), ab11+c52 (K3432), ab11+c53 (K3433), ab11+c54 (K3434), ab11+c55 (K3435), ab11+c56 (K3436), ab11+c57 (K3437), ab11+c58 (K3438), ab11+c59 (K3439), ab11+c60 (K3440), ab11+c61 (K3441), ab11+c62 (K3442), ab11+c63 (K3443), ab11+c64 (K3444), ab11+c65 (K3445), ab11+c66 (K3446), ab11+c67 (K3447), ab11+c68 (K3448), ab11+c69 (K3449), ab11+c70 (K3450), ab11+c71 (K3451), ab11+c72 (K3452), ab11+c73 (K3453), ab11+c74 (K3454), ab11+c75 (K3455), ab11+c76 (K3456), ab11+c77 (K3457), ab11+c78 (K3458), ab11+c79 (K3459), ab11+c80 (K3460), ab11+c81 (K3461), ab11+c82 (K3462), ab11+c83 (K3463), ab11+c84 (K3464), ab11+c85 (K3465), ab11+c86 (K3466), ab11+c87 (K3467), ab11+c88 (K3468), ab11+c89 (K3469), ab11+c90 (K3470), ab11+c91 (K3471), ab11+c92 (K3472), ab11+c93 (K3473), ab11+c94 (K3474), ab11+c95 (K3475), ab11+c96 (K3476), ab11+c97 (K3477), ab11+c98 (K3478), ab11+c99 (K3479), ab11+c100 (K3480), ab11+c101 (K3481), ab11+c102 (K3482), ab11+c103 (K3483), ab11+c104 (K3484), ab11+c105 (K3485), ab11+c106 (K3486), ab11+c107 (K3487), ab11+c108 (K3488), ab11+c109 (K3489), ab11+c110 (K3490), ab11+c111 (K3491), ab11+c112 (K3492), ab11+c113 (K3493), ab11+c114 (K3494), ab11+c115 (K3495), ab11+c116 (K3496), ab11+c117 (K3497), ab11+c118 (K3498), ab11+c119 (3499), ab11+c120 (K3500), ab11+c121 (K3501), ab11+c122 (K3502), ab11+c123 (K3503), ab11+c124 (K3504), ab11+c125 (K3505), ab11+c126 (K3506), ab11+c127 (K3507), ab11+c128 (K3508), ab11+c129 (K3509), ab11+c130 (K3510), ab11+c131 (K3511), ab11+c132 (K3512), ab11+c133 (K3513), ab11+c134 (K3514), ab11+c135 (K3515), ab11+c136 (K3516), ab11+c137 (K3517), ab11+c138 (K3518), ab11+c139 (K3519), ab11+c140 (K3520), ab11+c141 (K3521), ab11+c142 (K3522), ab11+c143 (K3523), ab11+c144 (K3524), ab11+c145 (K3525), ab11+c146 (K3526), ab11+c147 (K3527), ab11+c148 (K3528), ab11+c149 (K3529), ab11+c150 (K3530), ab11+c151 (K3531), ab11+c152 (K3532), ab11+c153 (K3533), ab11+c154 (K3534), ab11+c155 (K3535), ab11+c156 (K3536), ab11+c157 (K3537), ab11+c158 (K3538), ab11+c159 (K3539), ab11+c160 (K3540), ab11+c161 (K3541), ab11+c162 (K3542), ab11+c163 (K3543), ab12+c1 (K3544), ab12+c2 (K3545), ab12+c3 (K3546), ab12+c4 (K3547), ab12+c5 (K3548), ab12+c6 (K3549), ab12+c7 (K3550), ab12+c8 (K3551), ab12+c9 (K3552), ab12+c10 (K3553), ab12+c11 (K3554), ab12+c12 (K3555), ab12+c13 (K3556), ab12+c14 (K3557), ab12+c15 (K3558), ab12+c16 (K3559), ab12+c17 (K3560), ab12+c18 (K3561), ab12+c19 (K3562), ab12+c20 (K3563), ab12+c21 (K3564), ab12+c22 (K3565), ab12+c23 (K3566), ab12+c24 (K3567), ab12+c25 (K3568), ab12+c26 (K3569), ab12+c27 (K3570), ab12+c28 (K3571), ab12+c29 (K3572), ab12+c30 (K3573), ab12+c31 (K3574), ab12+c32 (K3575), ab12+c33 (K3576), ab12+c34 (K3577), ab12+c35 (K3578), ab12+c36 (K3579), ab12+c37 (K3580), ab12+c38 (K3581), ab12+c39 (K3582), ab12+c40 (K3583), ab12+c41 (K3584), ab12+c42 (K3585), ab12+c43 (K3586), ab12+c44 (K3587), ab12+c45 (K3588), ab12+c46 (K3589), ab12+c47 (K3590), ab12+c48 (K3591), ab12+c49 (K3592), ab12+c50 (K3593), ab12+c51 (K3594), ab12+c52 (K3595), ab12+c53 (K3596), ab12+c54 (K3597), ab12+c55 (K3598), ab12+c56 (K3599), ab12+c57 (K3600), ab12+c58 (K3601), ab12+c59 (K3602), ab12+c60 (K3603), ab12+c61 (K3604), ab12+c62 (K3605), ab12+c63 (K3606), ab12+c64 (K3607), ab12+c65 (K3608), ab12+c66 (K3609), ab12+c67 (K3610), ab12+c68 (K3611), ab12+c69 (K3612), ab12+c70 (K3613), ab12+c71 (K3614), ab12+c72 (K3615), ab12+c73 (K3616), ab12+c74 (K3617), ab12+c75 (K3618), ab12+c76 (K3619), ab12+c77 (K3620), ab12+c78 (K3621), ab12+c79 (K3622), ab12+c80 (K3623), ab12+c81 (K3624), ab12+c82 (K3625), ab12+c83 (K3626), ab12+c84 (K3527), ab12+c85 (K3628), ab12+c86 (K3629), ab12+c87 (K3630), ab12+c88 (K3631), ab12+c89 (K3632), ab12+c90 (K3533), ab12+c91 (K3634), ab12+c92 (K3635), ab12+c93 (K3636), ab12+c94 (K3637), ab12+c95 (K3638), ab12+c96 (K3639), ab12+c97 (K3640), ab12+c98 (K3641), ab12+c99 (K3642), ab12+c100 (K3643), ab12+c101 (K3644), ab12+c102 (K3645), ab12+c103 (K3646), ab12+c104 (K3647), ab12+c105 (K3648), ab12+c106 (K3649), ab12+c107 (K3650), ab12+c108 (K3651), ab12+c109 (K3652), ab12+c110 (K3653), ab12+c111 (K3654), ab12+c112 (K3655), ab12+c113 (K3656), ab12+c114 (K3657), ab12+c115 (K3658), ab12+c116 (K3659), ab12+c117 (K3660), ab12+c118 (K3661), ab12+c119 (K3662), ab12+c120 (K3663), ab12+c121 (K3664), ab12+c122 (K3665), ab12+c123 (K3666), ab12+c124 (K3667), ab12+c125 (K3668), ab12+c126 (K3669), ab12+c127 (K3670), ab12+c128 (K3671), ab12+c129 (K3672), ab12+c130 (K3673), ab12+c131 (K3674), ab12+c132 (K3675), ab12+c133 (K3676), ab12+c134 (K3677), ab12+c135 (K3678), ab12+c136 (K3679), ab12+c137 (K3680), ab12+c138 (K3681), ab12+c139 (K3682), ab12+c140 (K3683), ab12+c141 (K3684), ab12+c142 (K3685), ab12+c143 (K3686), ab12+c144 (K3687), ab12+c145 (K3688), ab12+c146 (K3689), ab12+c147 (K3690), ab12+c148 (K3691), ab12+c149 (K3692), ab12+c150 (K3693), ab12+c151 (K3694), ab12+c152 (K3695), ab12+c153 (K3696), ab12+c154 (K3697), ab12+c155 (K3698), ab12+c156 (K3699), ab12+c157 (K3700), ab12+c158 (K3701), ab12+c159 (K3702), ab12+c160 (K3703), ab12+c161 (K3704), ab12+c162 (K3705), ab12+c163 (K3706), ab13+c1 (K3707), ab13+c2 (K3708), ab13+c3 (K3709), ab13+c4 (K3710), ab13+c5 (K3711), ab13+c6 (K3712), ab13+c7 (K3713), ab13+c8 (K3714), ab13+c9 (K3715), ab13+c10 (K3716), ab13+c11 (K3717), ab13+c12 (K3718), ab13+c13 (K3719), ab13+c14 (K3720), ab13+c15 (K3721), ab13+c16 (K3722), ab13+c17 (K3723), ab13+c18 (K3724), ab13+c19 (K3725), ab13+c20 (K3726), ab13+c21 (K3727), ab13+c22 (K3728), ab13+c23 (K3729), ab13+c24 (K3730), ab13+c25 (K3731), ab13+c26 (K3732), ab13+c27 (K3733), ab13+c28 (K3734), ab13+c29 (K3735), ab13+c30 (K3736), ab13+c31 (K3737), ab13+c32 (K3738), ab13+c33 (K3739), ab13+c34 (K3740), ab13+c35 (K3741), ab13+c36 (K3742), ab13+c37 (K3743), ab13+c38 (K3744), ab13+c39 (K3745), ab13+c40 (K3746), ab13+c41 (K3747), ab13+c42 (K3748), ab13+c43 (K3749), ab13+c44 (K3750), ab13+c45 (K3751), ab13+c46 (K3752), ab13+c47 (K3753), ab13+c48 (K3754), ab13+c49 (K3755), ab13+c50 (K3756), ab13+c51 (K3757), ab13+c52 (K3758), ab13+c53 (K3759), ab13+c54 (K3760), ab13+c55 (K3761), ab13+c56 (K3762), ab13+c57 (K3763), ab13+c58 (K3764), ab13+c59 (K3765), ab13+c60 (K3766), ab13+c61 (K3767), ab13+c62 (K3768), ab13+c63 (K3769), ab13+c64 (K3770), ab13+c65 (K3771), ab13+c66 (K3772), ab13+c67 (K3773), ab13+c68 (K3774), ab13+c69 (K3775), ab13+c70 (K3776), ab13+c71 (K3777), ab13+c72 (K3778), ab13+c73 (K3779), ab13+c74 (K3780), ab13+c75 (K3781), ab13+c76 (K3782), ab13+c77 (K3783), ab13+c78 (K3784), ab13+c79 (K3785), ab13+c80 (K3786), ab13+c81 (K3787), ab13+c82 (K3788), ab13+c83 (K3789), ab13+c84 (K3790), ab13+c85 (K3791), ab13+c86 (K3792), ab13+c87 (K3793), ab13+c88 (K3794), ab13+c89 (K3795), ab13+c90 (K3796), ab13+c91 (K3797), ab13+c92 (K3798), ab13+c93 (K3799), ab13+c94 (K3800), ab13+c95 (K3801), ab13+c96 (K3802), ab13+c97 (K3803), ab13+c98 (K3804), ab13+c99 (K3805), ab13+c100 (K3806), ab13+c101 (K3807), ab13+c102 (K3808), ab13+c103 (K3809), ab13+c104 (K3810), ab13+c105 (K3811), ab13+c106 (K3812), ab13+c107 (K3813), ab13+c108 (K3814), ab13+c109 (K3815), ab13+c110 (K3816), ab13+c111 (K3817), ab13+c112 (K3818), ab13+c113 (K3819), ab13+c114 (K3820), ab13+c115 (K3821), ab13+c116 (K3822), ab13+c117 (K3823), ab13+c118 (K3824), ab13+c119 (K3825), ab13+c120 (K3826), ab13+c121 (K3827), ab13+c122 (K3828), ab13+c123 (K3829), ab13+c124 (K3830), ab13+c125 (K3831), ab13+c126 (K3832), ab13+c127 (K3833), ab13+c128 (K3834), ab13+c129 (K3835), ab13+c130 (K3836), ab13+c131 (K3837), ab13+c132 (K3838), ab13+c133 (K3839), ab13+c134 (K3840), ab13+c135 (K3841), ab13+c136 (K3842), ab13+c137 (K3843), ab13+c138 (K3844), ab13+c139 (K3845), ab13+c140 (K3846), ab13+c141 (K3847), ab13+c142 (K3848), ab13+c143 (K3849), ab13+c144 (K3850), ab13+c145 (K3851), ab13+c146 (K3852), ab13+c147 (K3853), ab13+c148 (K3854), ab13+c149 (K3855), ab13+c150 (K3856), ab13+c151 (K3857), ab13+c152 (K3858), ab13+c153 (K3859), ab13+c154 (K3860), ab13+c155 (K3861), ab13+c156 (K3862), ab13+c157 (K3863), ab13+c158 (K3864), ab13+c159 (K3865), ab13+c160 (K3866), ab13+c161 (K3867), ab13+c162 (K3868), ab13+c163 (K3869), ab14+c1 (K3870), ab14+c2 (K3871), ab14+c3 (K3872), ab14+c4 (K3873), ab14+c5 (K3874), ab14+c6 (K3875), ab14+c7 (K3876), ab14+c8 (K3877), ab14+c9 (K3878), ab14+c10 (K3879), ab14+c11 (K3880), ab14+c12 (K3881), ab14+c13 (K3882), ab14+c14 (K3883), ab14+c15 (K3884), ab14+c16 (K3885), ab14+c17 (K3886), ab14+c18 (K3887), ab14+c19 (K3888), ab14+c20 (K3889), ab14+c21 (K3890), ab14+c22 (K3891), ab14+c23 (K3892), ab14+c24 (3893), ab14+c25 (K3894), ab14+c26 (K3895), ab14+c27 (K3896), ab14+c28 (K3897), ab14+c29 (K3898), ab14+c30 (K3899), ab14+c31 (K3900), ab14+c32 (K3901), ab14+c33 (K3902), ab14+c34 (K3903), ab14+c35 (K3904), ab14+c36 (K3905), ab14+c37 (K3906), ab14+c38 (K3907), ab14+c39 (K3908), ab14+c40 (K3909), ab14+c41 (K3910), ab14+c42 (K3911), ab14+c43 (K3912), ab14+c44 (K3913), ab14+c45 (K3914), ab14+c46 (K3915), ab14+c47 (K3916), ab14+c48 (K3917), ab14+c49 (K3918), ab14+c50 (K3919), ab14+c51 (K3920), ab14+c52 (K3921), ab14+c53 (K3922), ab14+c54 (K3923), ab14+c55 (K3924), ab14+c56 (K3925), ab14+c57 (K3926), ab14+c58 (K3927), ab14+c59 (K3928), ab14+c60 (K3929), ab14+c61 (K3930), ab14+c62 (K3931), ab14+c63 (K3932), ab14+c64 (K3933), ab14+c65 (K3934), ab14+c66 (3935), ab14+c67 (K3936), ab14+c68 (K3937), ab14+c69 (K3938), ab14+c70 (K3939), ab14+c71 (K3940), ab14+c72 (K3941), ab14+c73 (K3942), ab14+c74 (K3943), ab14+c75 (K3944), ab14+c76 (K3945), ab14+c77 (K3946), ab14+c78 (K3947), ab14+c79 (K3948), ab14+c80 (K3949), ab14+c81 (K3950), ab14+c82 (K3951), ab14+c83 (K3952), ab14+c84 (K3953), ab14+c85 (K3954), ab14+c86 (K3955), ab14+c87 (K3956), ab14+c88 (K3957), ab14+c89 (K3958), ab14+c90 (K3959), ab14+c91 (K3960), ab14+c92 (K3961), ab14+c93 (K3962), ab14+c94 (K3963), ab14+c95 (K3964), ab14+c96 (K3965), ab14+c97 (K3966), ab14+c98 (K3967), ab14+c99 (K3968), ab14+c100 (K3969), ab14+c101 (K3970), ab14+c102 (K3971), ab14+c103 (K3972), ab14+c104 (K3973), ab14+c105 (K3974), ab14+c106 (K3975), ab14+c107 (K3976), ab14+c108 (K3977), ab14+c109 (K3978), ab14+c110 (K3979), ab14+c111 (K3980), ab14+c112 (K3981), ab14+c113 (K3982), ab14+c114 (K3983), ab14+c115 (K3984), ab14+c116 (K3985), ab14+c117 (K3986), ab14+c118 (K3987), ab14+c119 (K3988), ab14+c120 (K3989), ab14+c121 (K3990), ab14+c122 (K3991), ab14+c123 (K3992), ab14+c124 (K3993), ab14+c125 (K3994), ab14+c126 (K3995), ab14+c127 (K3996), ab14+c128 (K3997), ab14+c129 (K3998), ab14+c130 (K3999), ab14+c131 (K4000), ab14+c132 (K4001), ab14+c133 (K4002), ab14+c134 (K4003), ab14+c135 (K4004), ab14+c136 (K4005), ab14+c137 (K4006), ab14+c138 (K4007), ab14+c139 (K4008), ab14+c140 (K4009), ab14+c141 (K4010), ab14+c142 (K4011), ab14+c143 (K4012), ab14+c144 (K4013), ab14+c145 (K4014), ab14+c146 (K4015), ab14+c147 (K4016), ab14+c148 (K4017), ab14+c149 (K4018), ab14+c150 (K4019), ab14+c151 (K4020), ab14+c152 (K4021), ab14+c153 (K4022), ab14+c154 (K4023), ab14+c155 (K4024), ab14+c156 (K4025), ab14+c157 (K4026), ab14+c158 (K4027), ab14+c159 (K4028), ab14+c160 (K4029), ab14+c161 (K4030), ab14+c162 (K4031), ab14+c163 (K4032), ab15+c1 (K4033), ab15+c2 (K4034), ab15+c3 (K4035), ab15+c4 (K4036), ab15+c5 (K4037), ab15+c6 (K4038), ab15+c7 (K4039), ab15+c8 (K4040), ab15+c9 (K4041), ab15+c10 (K4042), ab15+c11 (K4043), ab15+c12 (K4044), ab15+c13 (K4045), ab15+c14 (K4046), ab15+c15 (K4047), ab15+c16 (K4048), ab15+c17 (K4049), ab15+c18 (K4050), ab15+c19 (K4051), ab15+c20 (K4052), ab15+c21 (K4053), ab15+c22 (K4054), ab15+c23 (K4055), ab15+c24 (K4056), ab15+c25 (K4057), ab15+c26 (K4058), ab15+c27 (K4059), ab15+c28 (K4060), ab15+c29 (K4061), ab15+c30 (K4062), ab15+c31 (K4063), ab15+c32 (K4064), ab15+c33 (K4065), ab15+c34 (K4066), ab15+c35 (K4067), ab15+c36 (K4068), ab15+c37 (K4069), ab15+c38 (K4070), ab15+c39 (K4071), ab15+c40 (K4072), ab15+c41 (K4073), ab15+c42 (K4074), ab15+c43 (K4075), ab15+c44 (K4076), ab15+c45 (K4077), ab15+c46 (K4078), ab15+c47 (K4079), ab15+c48 (K4080), ab15+c49 (K4081), ab15+c50 (K4082), ab15+c51 (K4083), ab15+c52 (K4084), ab15+c53 (K4085), ab15+c54 (K4086), ab15+c55 (K4087), ab15+c56 (K4088), ab15+c57 (K4089), ab15+c58 (K4090), ab15+c59 (K4091), ab15+c60 (K4092), ab15+c61 (K4093), ab15+c62 (K4094), ab15+c63 (K4095), ab15+c64 (K4096), ab15+c65 (K4097), ab15+c66 (K4098), ab15+c67 (K4099), ab15+c68 (K4100), ab15+c69 (K4101), ab15+c70 (K4102), ab15+c71 (K4103), ab15+c72 (K4104), ab15+c73 (K4105), ab15+c74 (K4106), ab15+c75 (K4107), ab15+c76 (K4108), ab15+c77 (K4109), ab15+c78 (K4110), ab15+c79 (K4111), ab15+c80 (K4112), ab15+c81 (K4113), ab15+c82 (K4114), ab15+c83 (K4115), ab15+c84 (K4116), ab15+c85 (K4117), ab15+c86 (K4118), ab15+c87 (K4119), ab15+c88 (K4120), ab15+c89 (K4121), ab15+c90 (K4122), ab15+c91 (K4123), ab15+c92 (K4124), ab15+c93 (K4125), ab15+c94 (K4126), ab15+c95 (K4127), ab15+c96 (K4128), ab15+c97 (K4129), ab15+c98 (K4130), ab15+c99 (K4131), ab15+c100 (K4132), ab15+c101 (K4133), ab15+c102 (K4134), ab15+c103 (K4135), ab15+c104 (K4136), ab15+c105 (K4137), ab15+c106 (K4138), ab15+c107 (K4139), ab15+c108 (K4140), ab15+c109 (K4141), ab15+c110 (K4142), ab15+c111 (K4143), ab15+c112 (K4144), ab15+c113 (K4145), ab15+c114 (K4146), ab15+c115 (K4147), ab15+c116 (K4148), ab15+c117 (K4149), ab15+c118 (K4150), ab15+c119 (K4151), ab15+c120 (K4152), ab15+c121 (K4153), ab15+c122 (K4154), ab15+c123 (K4155), ab15+c124 (K4156), ab15+c125 (K4157), ab15+c126 (K4158), ab15+c127 (K4159), ab15+c128 (K4160), ab15+c129 (K4161), ab15+c130 (K4162), ab15+c131 (K4163), ab15+c132 (K4164), ab15+c133 (K4165), ab15+c134 (K4166), ab15+c135 (K4167), ab15+c136 (K4168), ab15+c137 (4169), ab15+c138 (K4170), ab15+c139 (K4171), ab15+c140 (K4172), ab15+c141 (K4173), ab15+c142 (K4174), ab15+c143 (K4175), ab15+c144 (K4176), ab15+c145 (K4177), ab15+c146 (K4178), ab15+c147 (K4179), ab15+c148 (K4180), ab15+c149 (K4181), ab15+c150 (K4182), ab15+c151 (K4183), ab15+c152 (K4184), ab15+c153 (K4185), ab15+c154 (K4186), ab15+c155 (K4187), ab15+c156 (K4188), ab15+c157 (K4189), ab15+c158 (K4190), ab15+c159 (K4191), ab15+c160 (K4192), ab15+c161 (K4193), ab15+c162 (K4194), ab15+c163 (K4195), ab16+c1 (K4196), ab16+c2 (K4197), ab16+c3 (K4198), ab16+c4 (K4199), ab16+c5 (K4200), ab16+c6 (K4201), ab16+c7 (K4202), ab16+c8 (K4203), ab16+c9 (K4204), ab16+c10 (K4205), ab16+c11 (K4206), ab16+c12 (K4207), ab16+c13 (K4208), ab16+c14 (K4209), ab16+c15 (K4210), ab16+c16 (K4211), ab16+c17 (K4212), ab16+c18 (K4213), ab16+c19 (K4214), ab16+c20 (K4215), ab16+c21 (K4216), ab16+c22 (K4217), ab16+c23 (K4218), ab16+c24 (K4219), ab16+c25 (K4220), ab16+c26 (K4221), ab16+c27 (K4222), ab16+c28 (K4223), ab16+c29 (K4224), ab16+c30 (K4225), ab16+c31 (K4226), ab16+c32 (K4227), ab16+c33 (K4228), ab16+c34 (K4229), ab16+c35 (K4230), ab16+c36 (K4231), ab16+c37 (K4232), ab16+c38 (K4233), ab16+c39 (K4234), ab16+c40 (K4235), ab16+c41 (K4236), ab16+c42 (K4237), ab16+c43 (K4238), ab16+c44 (K4239), ab16+c45 (K4240), ab16+c46 (K4241), ab16+c47 (K4242), ab16+c48 (K4243), ab16+c49 (K4244), ab16+c50 (K4245), ab16+c51 (K4246), ab16+c52 (K4247), ab16+c53 (K4248), ab16+c54 (K4249), ab16+c55 (K4250), ab16+c56 (K4251), ab16+c57 (K4252), ab16+c58 (K4253), ab16+c59 (K4254), ab16+c60 (K4255), ab16+c61 (K4256), ab16+c62 (K4257), ab16+c63 (K4258), ab16+c64 (K4259), ab16+c65 (K4260), ab16+c66 (K4261), ab16+c67 (K4262), ab16+c68 (K4263), ab16+c69 (K4264), ab16+c70 (K4265), ab16+c71 (K4266), ab16+c72 (K4267), ab16+c73 (K4268), ab16+c74 (K4269), ab16+c75 (K4270), ab16+c76 (K4271), ab16+c77 (K4272), ab16+c78 (K4273), ab16+c79 (K4274), ab16+c80 (K4275), ab16+c81 (K4276), ab16+c82 (K4277), ab16+c83 (K4278), ab16+c84 (K4279), ab16+c85 (K4280), ab16+c86 (K4281), ab16+c87 (K4282), ab16+c88 (K4283), ab16+c89 (K4284), ab16+c90 (K4285), ab16+c91 (K4286), ab16+c92 (K4287), ab16+c93 (K4288), ab16+c94 (K4289), ab16+c95 (K4290), ab16+c96 (K4291), ab16+c97 (K4292), ab16+c98 (K4293), ab16+c99 (K4294), ab16+c100 (K4295), ab16+c101 (K4296), ab16+c102 (K4297), ab16+c103 (K4298), ab16+c104 (K4299), ab16+c105 (K4300), ab16+c106 (K4301), ab16+c107 (K4302), ab16+c108 (K4303), ab16+c109 (K4304), ab16+c110 (K4305), ab16+c111 (K4306), ab16+c112 (K4307), ab16+c113 (K4308), ab16+c114 (K4309), ab16+c115 (K4310), ab16+c116 (K4311), ab16+c117 (K4312), ab16+c118 (K4313), ab16+c119 (K4314), ab16+c120 (K4315), ab16+c121 (K4316), ab16+c122 (K4317), ab16+c123 (K4318), ab16+c124 (K4319), ab16+c125 (K4320), ab16+c126 (K4321), ab16+c127 (K4322), ab16+c128 (K4323), ab16+c129 (K4324), ab16+c130 (K4325), ab16+c131 (K4326), ab16+c132 (K4327), ab16+c133 (K4328), ab16+c134 (K4329), ab16+c135 (K4330), ab16+c136 (K4331), ab16+c137 (K4332), ab16+c138 (K4333), ab16+c139 (K4334), ab16+c140 (K4335), ab16+c141 (K4336), ab16+c142 (K4337), ab16+c143 (K4338), ab16+c144 (K4339), ab16+c145 (K4340), ab16+c146 (K4341), ab16+c147 (K4342), ab16+c148 (K4343), ab16+c149 (K4344), ab16+c150 (K4345), ab16+c151 (K4346), ab16+c152 (K4347), ab16+c153 (K4348), ab16+c154 (K4349), ab16+c155 (K4350), ab16+c156 (K4351), ab16+c157 (K4352), ab16+c158 (K4353), ab16+c159 (K4354), ab16+c160 (K4355), ab16+c161 (K4356), ab16+c162 (K4357), ab16+c163 (K4358), ab1+c164 (K5769), ab1+c165 (K5770), ab1+c166 (K5771), ab1+c167 (K5772), ab1+c168 (K5773), ab1+c169 (K5774), ab1+c170 (K5775), ab1+c171 (K5776), ab1+c172 (K5777), ab1+c173 (K5778), ab1+c174 (K5779), ab1+c175 (K5780), ab1+c176 (K5781), ab1+c177 (K5782), ab1+c178 (K5783), ab1+c179 (K5784), ab1+c180 (K5785), ab1+c181 (K5786), ab1+c182 (K5787), ab1+c183 (K5788), ab1+c184 (K5789), ab1+c185 (K5790), ab1+c186 (K5791), ab1+c187 (K5792), ab1+c188 (K5793), ab1+c189 (K5794), ab1+c190 (K5795), ab1+c191 (K5796), ab1+c192 (K5797), ab1+c193 (K5798), ab1+c194 (K5799), ab1+c195 (K5800), ab1+c196 (K5801), ab1+c197 (K5802), ab1+c198 (K5803), ab1+c199 (K5804), ab1+c200 (K5805), ab2+c164 (K5806), ab2+c165 (K5807), ab2+c166 (K5808), ab2+c167 (K5809), ab2+c168 (K5810), ab2+c169 (K5811), ab2+c170 (K5812), ab2+c171 (K5813), ab2+c172 (K5814), ab2+c173 (K5815), ab2+c174 (K5816), ab2+c175 (K5817), ab2+c176 (K5818), ab2+c177 (K5819), ab2+c178 (K5820), ab2+c179 (K5821), ab2+c180 (K5822), ab2+c181 (K5823), ab2+c182 (K5824), ab2+c183 (K5825), ab2+c184 (K5826), ab2+c185 (K5827), ab2+c186 (K5828), ab2+c187 (K5829), ab2+c188 (K5830), ab2+c189 (K5831), ab2+c190 (K5832), ab2+c191 (K5833), ab2+c192 (K5834), ab2+c193 (K5835), ab2+c194 (K5836), ab2+c195 (K5837), ab2+c196 (K5838), ab2+c197 (K5839), ab2+c198 (K5840), ab2+c199 (K5841), ab2+c200 (K5842), ab3+c164 (K5843), ab3+c165 (K5844), ab3+c166 (K5845), ab3+c167 (K5846), ab3+c168 (K5847), ab3+c169 (K5848), ab3+c170 (K5849), ab3+c171 (K5850), ab3+c172 (K5851), ab3+c173 (K5852), ab3+c174 (K5853), ab3+c175 (K5854), ab3+c176 (K5855), ab3+c177 (K5856), ab3+c178 (K5857), ab3+c179 (K5858), ab3+c180 (K5859), ab3+c181 (K5860), ab3+c182 (K5861), ab3+c183 (K5862), ab3+c184 (K5863), ab3+c185 (K5864), ab3+c186 (K5865), ab3+c187 (K5866), ab3+c188 (K5867), ab3+c189 (K5868), ab3+c190 (K5869), ab3+c191 (K5870), ab3+c192 (K5871), ab3+c193 (K5872), ab3+c194 (K5873), ab3+c195 (K5874), ab3+c196 (K5875), ab3+c197 (K5876), ab3+c198 (K5877), ab3+c199 (K5878), ab3+c200 (K5879), ab4+c164 (K5880), ab4+c165 (K5881), ab4+c166 (K5882), ab4+c167 (K5883), ab4+c168 (K5884), ab4+c169 (K5885), ab4+c170 (K5886), ab4+c171 (K5887), ab4+c172 (K5888), ab4+c173 (K5889), ab4+c174 (K5890), ab4+c175 (K5891), ab4+c176 (K5892), ab4+c177 (K5893), ab4+c178 (K5894), ab4+c179 (K5895), ab4+c180 (K5896), ab4+c181 (K5897), ab4+c182 (K5898), ab4+c183 (K5899), ab4+c184 (K5900), ab4+c185 (K5901), ab4+c186 (K5902), ab4+c187 (K5903), ab4+c188 (K5904), ab4+c189 (K5905), ab4+c190 (K5906), ab4+c191 (K5907), ab4+c192 (K5908), ab4+c193 (K5909), ab4+c194 (K5910), ab4+c195 (K5911), ab4+c196 (K5912), ab4+c197 (K5913), ab4+c198 (K5914), ab4+c199 (K5915), ab4+c200 (K5916), ab5+c164 (K5917), ab5+c165 (K5918), ab5+c166 (K5919), ab5+c167 (K5920), ab5+c168 (K5921), ab5+c169 (K5922), ab5+c170 (K5923), ab5+c171 (K5924), ab5+c172 (K5925), ab5+c173 (K5926), ab5+c174 (K5927), ab5+c175 (K5928), ab5+c176 (K5929), ab5+c177 (K5930), ab5+c178 (K5931), ab5+c179 (K5932), ab5+c180 (K5933), ab5+c181 (K5934), ab5+c182 (K5935), ab5+c183 (K5936), ab5+c184 (K5937), ab5+c185 (K5938), ab5+c186 (K5939), ab5+c187 (K5940), ab5+c188 (K5941), ab5+c189 (K5942), ab5+c190 (K5943), ab5+c191 (K5944), ab5+c192 (K5945), ab5+c193 (K5946), ab5+c194 (K5947), ab5+c195 (K5948), ab5+c196 (K5949), ab5+c197 (K5950), ab5+c198 (K5951), ab5+c199 (K5952), ab5+c200 (K5953), ab6+c164 (K5954), ab6+c165 (K5955), ab6+c166 (K5956), ab6+c167 (K5957), ab6+c168 (K5958), ab6+c169 (K5959), ab6+c170 (K5960), ab6+c171 (K5961), ab6+c172 (K5962), ab6+c173 (K5963), ab6+c174 (K5964), ab6+c175 (K5965), ab6+c176 (K5966), ab6+c177 (K5967), ab6+c178 (K5968), ab6+c179 (K5969), ab6+c180 (K5970), ab6+c181 (K5971), ab6+c182 (K5972), ab6+c183 (K5973), ab6+c184 (K5974), ab6+c185 (K5975), ab6+c186 (K5976), ab6+c187 (K5977), ab6+c188 (K5978), ab6+c189 (K5979), ab6+c190 (K5980), ab6+c191 (K5981), ab6+c192 (K5982), ab6+c193 (K5983), ab6+c194 (K5984), ab6+c195 (K5985), ab6+c196 (K5986), ab6+c197 (K5987), ab6+c198 (K5988), ab6+c199 (K5989), ab6+c200 (K5990), ab7+c164 (K5991), ab7+c165 (K5992), ab7+c166 (K5993), ab7+c167 (K5994), ab7+c168 (K5995), ab7+c169 (K5996), ab7+c170 (K5997), ab7+c171 (K5998), ab7+c172 (K5999), ab7+c173 (K6000), ab7+c174 (K6001), ab7+c175 (K6002), ab7+c176 (K6003), ab7+c177 (K6004), ab7+c178 (K6005), ab7+c179 (K6006), ab7+c180 (K6007), ab7+c181 (K6008), ab7+c182 (K6009), ab7+c183 (K6010), ab7+c184 (K6011), ab7+c185 (K6012), ab7+c186 (K6013), ab7+c187 (K6014), ab7+c188 (K6015), ab7+c189 (K6016), ab7+c190 (K6017), ab7+c191 (K6018), ab7+c192 (K6019), ab7+c193 (K6020), ab7+c194 (K6021), ab7+c195 (K6022), ab7+c196 (K6023), ab7+c197 (K6024), ab7+c198 (K6025), ab7+c199 (K6026), ab7+c200 (K6027), ab8+c164 (K6028), ab8+c165 (K6029), ab8+c166 (K6030), ab8+c167 (K6031), ab8+c168 (K6032), ab8+c169 (K6033), ab8+c170 (K6034), ab8+c171 (K6035), ab8+c172 (K6036), ab8+c173 (K6037), ab8+c174 (K6038), ab8+c175 (K6039), ab8+c176 (K6040), ab8+c177 (K6041), ab8+c178 (K6042), ab8+c179 (K6043), ab8+c180 (K6044), ab8+c181 (K6045), ab8+c182 (K6046), ab8+c183 (K6047), ab8+c184 (K6048), ab8+c185 (K6049), ab8+c186 (K6050), ab8+c187 (K6051), ab8+c188 (K6052), ab8+c189 (K6053), ab8+c190 (K6054), ab8+c191 (K6055), ab8+c192 (K6056), ab8+c193 (K6057), ab8+c194 (K6058), ab8+c195 (K6059), ab8+c196 (K6060), ab8+c197 (K6061), ab8+c198 (K6062), ab8+c199 (K6063), ab8+c200 (K6064), ab9+c164 (K6065), ab9+c165 (K6066), ab9+c166 (K6067), ab9+c167 (K6068), ab9+c168 (K6069), ab9+c169 (K6070), ab9+c170 (K6071), ab9+c171 (K6072), ab9+c172 (K6073), ab9+c173 (K6074), ab9+c174 (K6075), ab9+c175 (K6076), ab9+c176 (K6077), ab9+c177 (K6078), ab9+c178 (K6079), ab9+c179 (K6080), ab9+c180 (K6081), ab9+c181 (K6082), ab9+c182 (K6083), ab9+c183 (K6084), ab9+c184 (K6085), ab9+c185 (K6086), ab9+c186 (K6087), ab9+c187 (K6088), ab9+c188 (K6089), ab9+c189 (K6090), ab9+c190 (K6091), ab9+c191 (K6092), ab9+c192 (K6093), ab9+c193 (K6094), ab9+c194 (K6095), ab9+c195 (K6096), ab9+c196 (K6097), ab9+c197 (K6098), ab9+c198 (K6099), ab9+c199 (K6100), ab9+c200 (K6101), ab10+c164 (K6102), ab10+c165 (K6103), ab10+c166 (K6104), ab10+c167 (K6105), ab10+c168 (K6106), ab10+c169 (K6107), ab10+c170 (K6108), ab10+c171 (K6109), ab10+c172 (K6110), ab10+c173 (K6111), ab10+c174 (K6112), ab10+c175 (K6113), ab10+c176 (K6114), ab10+c177 (K6115), ab10+c178 (K6116), ab10+c179 (K6117), ab10+c180 (K6118), ab10+c181 (K6119), ab10+c182 (K6120), ab10+c183 (K6121), ab10+c184 (K6122), ab10+c185 (K6123), ab10+c186 (K6124), ab10+c187 (K6125), ab10+c188 (K6126), ab10+c189 (K6127), ab10+c190 (K6128), ab10+c191 (K6129), ab10+c192 (K6130), ab10+c193 (K6131), ab10+c194 (K6132), ab10+c195 (K6133), ab10+c196 (K6134), ab10+c197 (K6135), ab10+c198 (K6136), ab10+c199 (K6137), ab10+c200 (K6138), ab11+c164 (K6139), ab11+c165 (K6140), ab11+c166 (K6141), ab11+c167 (K6142), ab11+c168 (K6143), ab11+c169 (K6144), ab11+c170 (K6145), ab11+c171 (K6146), ab11+c172 (K6147), ab11+c173 (K6148), ab11+c174 (K6149), ab11+c175 (K6150), ab11+c176 (K6151), ab11+c177 (K6152), ab11+c178 (K6153), ab11+c179 (K6154), ab11+c180 (K6155), ab11+c181 (K6156), ab11+c182 (K6157), ab11+c183 (K6158), ab11+c184 (K6159), ab11+c185 (K6160), ab11+c186 (K6161), ab11+c187 (K6162), ab11+c188 (K6163), ab11+c189 (K6164), ab11+c190 (K6165), ab11+c191 (K6166), ab11+c192 (K6167), ab11+c193 (K6168), ab11+c194 (K6169), ab11+c195 (K6170), ab11+c196 (K6171), ab11+c197 (K6172), ab11+c198 (K6173), ab11+c199 (K6174), ab11+c200 (K6175), ab12+c164 (K6176), ab12+c165 (K6177), ab12+c166 (K6178), ab12+c167 (K6179), ab12+c168 (K6180), ab12+c169 (K6181), ab12+c170 (K6182), ab12+c171 (K6183), ab12+c172 (K6184), ab12+c173 (K6185), ab12+c174 (K6186), ab12+c175 (K6187), ab12+c176 (K6188), ab12+c177 (K6189), ab12+c178 (K6190), ab12+c179 (K6191), ab12+c180 (K6192), ab12+c181 (K6193), ab12+c182 (K6194), ab12+c183 (K6195), ab12+c184 (K6196), ab12+c185 (K6197), ab12+c186 (K6198), ab12+c187 (K6199), ab12+c188 (K6200), ab12+c189 (K6201), ab12+c190 (K6202), ab12+c191 (K6203), ab12+c192 (K6204), ab12+c193 (K6205), ab12+c194 (K6206), ab12+c195 (K6207), ab12+c196 (K6208), ab12+c197 (K6209), ab12+c198 (K6210), ab12+c199 (K6211), ab12+c200 (K6212), ab13+c164 (K6213), ab13+c165 (K6214), ab13+c166 (K6215), ab13+c167 (K6216), ab13+c168 (K6217), ab13+c169 (K6218), ab13+c170 (K6219), ab13+c171 (K6220), ab13+c172 (K6221), ab13+c173 (K6222), ab13+c174 (K6223), ab13+c175 (K6224), ab13+c176 (K6225), ab13+c177 (K6226), ab13+c178 (K6227), ab13+c179 (K6228), ab13+c180 (K6229), ab13+c181 (K6230), ab13+c182 (K6231), ab13+c183 (K6232), ab13+c184 (K6233), ab13+c185 (K6234), ab13+c186 (K6235), ab13+c187 (K6236), ab13+c188 (K6237), ab13+c189 (K6238), ab13+c190 (K6239), ab13+c191 (K6240), ab13+c192 (K6241), ab13+c193 (K6242), ab13+c194 (K6243), ab13+c195 (K6244), ab13+c196 (K6245), ab13+c197 (K6246), ab13+c198 (K6247), ab13+c199 (K6248), ab13+c200 (K6249), ab14+c164 (K6250), ab14+c165 (K6251), ab14+c166 (K6252), ab14+c167 (K6253), ab14+c168 (K6254), ab14+c169 (K6255), ab14+c170 (K6256), ab14+c171 (K6257), ab14+c172 (K6258), ab14+c173 (K6259), ab14+c174 (K6260), ab14+c175 (K6261), ab14+c176 (K6262), ab14+c177 (K6263), ab14+c178 (K6264), ab14+c179 (K6265), ab14+c180 (K6266), ab14+c181 (K6267), ab14+c182 (K6268), ab14+c183 (K6269), ab14+c184 (K6270), ab14+c185 (K6271), ab14+c186 (K6272), ab14+c187 (K6273), ab14+c188 (K6274), ab14+c189 (K6275), ab14+c190 (K6276), ab14+c191 (K6277), ab14+c192 (K6278), ab14+c193 (K6279), ab14+c194 (K6280), ab14+c195 (K6281), ab14+c196 (K6282), ab14+c197 (K6283), ab14+c198 (K6284), ab14+c199 (K6285), ab14+c200 (K6286), ab15+c164 (K6287), ab15+c165 (K6288), ab15+c166 (K6289), ab15+c167 (K6290), ab15+c168 (K6291), ab15+c169 (K6292), ab15+c170 (K6293), ab15+c171 (K6294), ab15+c172 (K6295), ab15+c173 (K6296), ab15+c174 (K6297), ab15+c175 (K6298), ab15+c176 (K6299), ab15+c177 (K6300), ab15+c178 (K6301), ab15+c179 (K6302), ab15+c180 (K6303), ab15+c181 (K6304), ab15+c182 (K6305), ab15+c183 (K6306), ab15+c184 (K6307), ab15+c185 (K6308), ab15+c186 (K6309), ab15+c187 (K6310), ab15+c188 (K6311), ab15+c189 (K6312), ab15+c190 (K6313), ab15+c191 (K6314), ab15+c192 (K6315), ab15+c193 (K6316), ab15+c194 (K6317), ab15+c195 (K6318), ab15+c196 (K6319), ab15+c197 (K6320), ab15+c198 (K6321), ab15+c199 (K6322), ab15+c200 (K6323), ab16+c164 (K6324), ab16+c165 (K6325), ab16+c166 (K6326), ab16+c167 (K6327), ab16+c168 (K6328), ab16+c169 (K6329), ab16+c170 (K6330), ab16+c171 (K6331), ab16+c172 (K6332), ab16+c173 (K6333), ab16+c174 (K6334), ab16+c175 (K6335), ab16+c176 (K6336), ab16+c177 (K6337), ab16+c178 (K6338), ab16+c179 (K6339), ab16+c180 (K6340), ab16+c181 (K6341), ab16+c182 (K6342), ab16+c183 (K6343), ab16+c184 (K6344), ab16+c185 (K6345), ab16+c186 (K6346), ab16+c187 (K6347), ab16+c188 (K6348), ab16+c189 (K6349), ab16+c190 (K6350), ab16+c191 (K6351), ab16+c192 (K6352), ab16+c193 (K6353), ab16+c194 (K6354), ab16+c195 (K6355), ab16+c196 (K6356), ab16+c197 (K6357), ab16+c198 (K6358), ab16+c199 (K6359), ab16+c200 (K6360), ab17+c1 (K6361), ab17+c2 (K6362), ab17+c3 (K6363), ab17+c4 (K6364), ab17+c5 (K6365), ab17+c6 (K6366), ab17+c7 (K6367), ab17+c8 (K6368), ab17+c9 (K6369), ab17+c10 (K6370), ab17+c11 (K6371), ab17+c12 (K6372), ab17+c13 (K6373), ab17+c14 (K6374), ab17+c15 (K6375), ab17+c16 (K6376), ab17+c17 (K6377), ab17+c18 (K6378), ab17+c19 (K6379), ab17+c20 (K6380), ab17+c21 (K6381), ab17+c22 (K6382), ab17+c23 (K6383), ab17+c24 (K6384), ab17+c25 (K6385), ab17+c26 (K6386), ab17+c27 (K6387), ab17+c28 (K6388), ab17+c29 (K6389), ab17+c30 (K6390), ab17+c31 (K6391), ab17+c32 (K6392), ab17+c33 (K6393), ab17+c34 (K6394), ab17+c35 (K6395), ab17+c36 (K6396), ab17+c37 (K6397), ab17+c38 (K6398), ab17+c39 (K6399), ab17+c40 (K6400), ab17+c41 (K6401), ab17+c42 (K6402), ab17+c43 (K6403), ab17+c44 (K6404), ab17+c45 (K6405), ab17+c46 (K6406), ab17+c47 (K6407), ab17+c48 (K6408), ab17+c49 (K6409), ab17+c50 (K6410), ab17+c51 (K6411), ab17+c52 (K6412), ab17+c53 (K6413), ab17+c54 (K6414), ab17+c55 (K6415), ab17+c56 (K6416), ab17+c57 (K6417), ab17+c58 (K6418), ab17+c59 (K6419), ab17+c60 (K6420), ab17+c61 (K6421), ab17+c62 (K6422), ab17+c63 (K6423), ab17+c64 (K6424), ab17+c65 (K6425), ab17+c66 (K6426), ab17+c67 (K6427), ab17+c68 (K6428), ab17+c69 (K6429), ab17+c70 (K6430), ab17+c71 (K6431), ab17+c72 (K6432), ab17+c73 (K6433), ab17+c74 (K6434), ab17+c75 (K6435), ab17+c76 (K6436), ab17+c77 (K6437), ab17+c78 (K6438), ab17+c79 (K6439), ab17+c80 (K6440), ab17+c81 (K6441), ab17+c82 (K6442), ab17+c83 (K6443), ab17+c84 (K6444), ab17+c85 (K6445), ab17+c86 (K6446), ab17+c87 (K6447), ab17+c88 (K6448), ab17+c89 (K6449), ab17+c90 (K6450), ab17+c91 (K6451), ab17+c92 (K6452), ab17+c93 (K6453), ab17+c94 (K6454), ab17+c95 (K6455), ab17+c96 (K6456), ab17+c97 (K6457), ab17+c98 (K6458), ab17+c99 (K6459), ab17+c100 (K6460), ab17+c101 (K6461), ab17+c102 (K6462), ab17+c103 (K6463), ab17+c104 (K6464), ab17+c105 (K6465), ab17+c106 (K6466), ab17+c107 (K6467), ab17+c108 (K6468), ab17+c109 (K6469), ab17+c110 (K6470), ab17+c111 (K6471), ab17+c112 (K6472), ab17+c113 (K6473), ab17+c114 (K6474), ab17+c115 (K6475), ab17+c116 (K6476), ab17+c117 (K6477), ab17+c118 (K6478), ab17+c119 (K6479), ab17+c120 (K6480), ab17+c121 (K6481), ab17+c122 (K6482), ab17+c123 (K6483), ab17+c124 (K6484), ab17+c125 (K6485), ab17+c126 (K6486), ab17+c127 (K6487), ab17+c128 (K6488), ab17+c129 (K6489), ab17+c130 (K6490), ab17+c131 (K6491), ab17+c132 (K6492), ab17+c133 (K6493), ab17+c134 (K6494), ab17+c135 (K6495), ab17+c136 (K6496), ab17+c137 (K6497), ab17+c138 (K6498), ab17+c139 (K6499), ab17+c140 (K6500), ab17+c141 (K6501), ab17+c142 (K6502), ab17+c143 (K6503), ab17+c144 (K6504), ab17+c145 (K6505), ab17+c146 (K6506), ab17+c147 (K6507), ab17+c148 (K6508), ab17+c149 (K6509), ab17+c150 (K6510), ab17+c151 (K6511), ab17+c152 (K6512), ab17+c153 (K6513), ab17+c154 (K6514), ab17+c155 (K6515), ab17+c156 (K6516), ab17+c157 (K6517), ab17+c158 (K6518), ab17+c159 (K6519), ab17+c160 (K6520), ab17+c161 (K6521), ab17+c162 (K6522), ab17+c163 (K6523), ab17+c164 (K6524), ab17+c165 (K6525), ab17+c166 (K6526), ab17+c167 (K6527), ab17+c168 (K6528), ab17+c169 (K6529), ab17+c170 (K6530), ab17+c171 (K6531), ab17+c172 (K6532), ab17+c173 (K6533), ab17+c174 (K6534), ab17+c175 (K6535), ab17+c176 (K6536), ab17+c177 (K6537), ab17+c178 (K6538), ab17+c179 (K6539), ab17+c180 (K6540), ab17+c181 (K6541), ab17+c182 (K6542), ab17+c183 (K6543), ab17+c184 (K6544), ab17+c185 (K6545), ab17+c186 (K6546), ab17+c187 (K6547), ab17+c188 (K6548), ab17+c189 (K6549), ab17+c190 (K6550), ab17+c191 (K6551), ab17+c192 (K6552), ab17+c193 (K6553), ab17+c194 (K6554), ab17+c195 (K6555), ab17+c196 (K6556), ab17+c197 (K6557), ab17+c198 (K6558), ab17+c199 (K6559), ab17+c200 (K6560), ab18+c1 (K6561), ab18+c2 (K6562), ab18+c3 (K6563), ab18+c4 (K6564), ab18+c5 (K6565), ab18+c6 (K6566), ab18+c7 (K6567), ab18+c8 (K6568), ab18+c9 (K6569), ab18+c10 (K6570), ab18+c11 (K6571), ab18+c12 (K6572), ab18+c13 (K6573), ab18+c14 (K6574), ab18+c15 (K6575), ab18+c16 (K6576), ab18+c17 (K6577), ab18+c18 (K6578), ab18+c19 (K6579), ab18+c20 (K6580), ab18+c21 (K6581), ab18+c22 (K6582), ab18+c23 (K6583), ab18+c24 (K6584), ab18+c25 (K6585), ab18+c26 (K6586), ab18+c27 (K6587), ab18+c28 (K6588), ab18+c29 (K6589), ab18+c30 (K6590), ab18+c31 (K6591), ab18+c32 (K6592), ab18+c33 (K6593), ab18+c34 (K6594), ab18+c35 (K6595), ab18+c36 (K6596), ab18+c37 (K6597), ab18+c38 (K6598), ab18+c39 (K6599), ab18+c40 (K6600), ab18+c41 (K6601), ab18+c42 (K6602), ab18+c43 (K6603), ab18+c44 (K6604), ab18+c45 (K6605), ab18+c46 (K6606), ab18+c47 (K6607), ab18+c48 (K6608), ab18+c49 (K6609), ab18+c50 (K6610), ab18+c51 (K6611), ab18+c52 (K6612), ab18+c53 (K6613), ab18+c54 (K6614), ab18+c55 (K6615), ab18+c56 (K6616), ab18+c57 (K6617), ab18+c58 (K6618), ab18+c59 (K6619), ab18+c60 (K6620), ab18+c61 (K6621), ab18+c62 (K6622), ab18+c63 (K6623), ab18+c64 (K6624), ab18+c65 (K6625), ab18+c66 (K6626), ab18+c67 (K6627), ab18+c68 (K6628), ab18+c69 (K6629), ab18+c70 (K6630), ab18+c71 (K6631), ab18+c72 (K6632), ab18+c73 (K6633), ab18+c74 (K6634), ab18+c75 (K6635), ab18+c76 (K6636), ab18+c77 (K6637), ab18+c78 (K6638), ab18+c79 (K6639), ab18+c80 (K6640), ab18+c81 (K6641), ab18+c82 (K6642), ab18+c83 (K6643), ab18+c84 (K6644), ab18+c85 (K6645), ab18+c86 (K6646), ab18+c87 (K6647), ab18+c88 (K6648), ab18+c89 (K6649), ab18+c90 (K6650), ab18+c91 (K6651), ab18+c92 (K6652), ab18+c93 (K6653), ab18+c94 (K6654), ab18+c95 (K6655), ab18+c96 (K6656), ab18+c97 (K6657), ab18+c98 (K6658), ab18+c99 (K6659), ab18+c100 (K6660), ab18+c101 (K6661), ab18+c102 (K6662), ab18+c103 (K6663), ab18+c104 (K6664), ab18+c105 (K6665), ab18+c106 (K6666), ab18+c107 (K6667), ab18+c108 (K6668), ab18+c109 (K6669), ab18+c110 (K6670), ab18+c111 (K6671), ab18+c112 (K6672), ab18+c113 (K6673), ab18+c114 (K6674), ab18+c115 (K6675), ab18+c116 (K6676), ab18+c117 (K6677), ab18+c118 (K6678), ab18+c119 (K6679), ab18+c120 (K6680), ab18+c121 (K6681), ab18+c122 (K6682), ab18+c123 (K6683), ab18+c124 (K6684), ab18+c125 (K6685), ab18+c126 (K6686), ab18+c127 (K6687), ab18+c128 (K6688), ab18+c129 (K6689), ab18+c130 (K6690), ab18+c131 (K6691), ab18+c132 (K6692), ab18+c133 (K6693), ab18+c134 (K6694), ab18+c135 (K6695), ab18+c136 (K6696), ab18+c137 (K6697), ab18+c138 (K6698), ab18+c139 (K6699), ab18+c140 (K6700), ab18+c141 (K6701), ab18+c142 (K6702), ab18+c143 (K6703), ab18+c144 (K6704), ab18+c145 (K6705), ab18+c146 (K6706), ab18+c147 (K6707), ab18+c148 (K6708), ab18+c149 (K6709), ab18+c150 (K6710), ab18+c151 (K6711), ab18+c152 (K6712), ab18+c153 (K6713), ab18+c154 (K6714), ab18+c155 (K6715), ab18+c156 (K6716), ab18+c157 (K6717), ab18+c158 (K6718), ab18+c159 (K6719), ab18+c160 (K6720), ab18+c161 (K6721), ab18+c162 (K6722), ab18+c163 (K6723), ab18+c164 (K6724), ab18+c165 (K6725), ab18+c166 (K6726), ab18+c167 (K6727), ab18+c168 (K6728), ab18+c169 (K6729), ab18+c170 (K6730), ab18+c171 (K6731), ab18+c172 (K6732), ab18+c173 (K6733), ab18+c174 (K6734), ab18+c175 (K6735), ab18+c176 (K6736), ab18+c177 (K6737), ab18+c178 (K6738), ab18+c179 (K6739), ab18+c180 (K6740), ab18+c181 (K6741), ab18+c182 (K6742), ab18+c183 (K6743), ab18+c184 (K6744), ab18+c185 (K6745), ab18+c186 (K6746), ab18+c187 (K6747), ab18+c188 (K6748), ab18+c189 (K6749), ab18+c190 (K6750), ab18+c191 (K6751), ab18+c192 (K6752), ab18+c193 (K6753), ab18+c194 (K6754), ab18+c195 (K6755), ab18+c196 (K6756), ab18+c197 (K6757), ab18+c198 (K6758), ab18+c199 (K6759), ab18+c200 (K6760), ab19+c1 (K6761), ab19+c2 (K6762), ab19+c3 (K6763), ab19+c4 (K6764), ab19+c5 (K6765), ab19+c6 (K6766), ab19+c7 (K6767), ab19+c8 (K6768), ab19+c9 (K6769), ab19+c10 (K6770), ab19+c11 (K6771), ab19+c12 (K6772), ab19+c13 (K6773), ab19+c14 (K6774), ab19+c15 (K6775), ab19+c16 (K6776), ab19+c17 (K6777), ab19+c18 (K6778), ab19+c19 (K6779), ab19+c20 (K6780), ab19+c21 (K6781), ab19+c22 (K6782), ab19+c23 (K6783), ab19+c24 (K6784), ab19+c25 (K6785), ab19+c26 (K6786), ab19+c27 (K6787), ab19+c28 (K6788), ab19+c29 (K6789), ab19+c30 (K6790), ab19+c31 (K6791), ab19+c32 (K6792), ab19+c33 (K6793), ab19+c34 (K6794), ab19+c35 (K6795), ab19+c36 (K6796), ab19+c37 (K6797), ab19+c38 (K6798), ab19+c39 (K6799), ab19+c40 (K6800), ab19+c41 (K6801), ab19+c42 (K6802), ab19+c43 (K6803), ab19+c44 (K6804), ab19+c45 (K6805), ab19+c46 (K6806), ab19+c47 (K6807), ab19+c48 (K6808), ab19+c49 (K6809), ab19+c50 (K6810), ab19+c51 (K6811), ab19+c52 (K6812), ab19+c53 (K6813), ab19+c54 (K6814), ab19+c55 (K6815), ab19+c56 (K6816), ab19+c57 (K6817), ab19+c58 (K6818), ab19+c59 (K6819), ab19+c60 (K6820), ab19+c61 (K6821), ab19+c62 (K6822), ab19+c63 (K6823), ab19+c64 (K6824), ab19+c65 (K6825), ab19+c66 (K6826), ab19+c67 (K6827), ab19+c68 (K6828), ab19+c69 (K6829), ab19+c70 (K6830), ab19+c71 (K6831), ab19+c72 (K6832), ab19+c73 (K6833), ab19+c74 (K6834), ab19+c75 (K6835), ab19+c76 (K6836), ab19+c77 (K6837), ab19+c78 (K6838), ab19+c79 (K6839), ab19+c80 (K6840), ab19+c81 (K6841), ab19+c82 (K6842), ab19+c83 (K6843), ab19+c84 (K6844), ab19+c85 (K6845), ab19+c86 (K6846), ab19+c87 (K6847), ab19+c88 (K6848), ab19+c89 (K6849), ab19+c90 (K6850), ab19+c91 (K6851), ab19+c92 (K6852), ab19+c93 (K6853), ab19+c94 (K6854), ab19+c95 (K6855), ab19+c96 (K6856), ab19+c97 (K6857), ab19+c98 (K6858), ab19+c99 (K6859), ab19+c100 (K6860), ab19+c101 (K6861), ab19+c102 (K6862), ab19+c103 (K6863), ab19+c104 (K6864), ab19+c105 (K6865), ab19+c106 (K6866), ab19+c107 (K6867), ab19+c108 (K6868), ab19+c109 (K6869), ab19+c110 (K6870), ab19+c111 (K6871), ab19+c112 (K6872), ab19+c113 (K6873), ab19+c114 (K6874), ab19+c115 (K6875), ab19+c116 (K6876), ab19+c117 (K6877), ab19+c118 (K6878), ab19+c119 (K6879), ab19+c120 (K6880), ab19+c121 (K6881), ab19+c122 (K6882), ab19+c123 (K6883), ab19+c124 (K6884), ab19+c125 (K6885), ab19+c126 (K6886), ab19+c127 (K6887), ab19+c128 (K6888), ab19+c129 (K6889), ab19+c130 (K6890), ab19+c131 (K6891), ab19+c132 (K6892), ab19+c133 (K6893), ab19+c134 (K6894), ab19+c135 (K6895), ab19+c136 (K6896), ab19+c137 (K6897), ab19+c138 (K6898), ab19+c139 (K6899), ab19+c140 (K6900), ab19+c141 (K6901), ab19+c142 (K6902), ab19+c143 (K6903), ab19+c144 (K6904), ab19+c145 (K6905), ab19+c146 (K6906), ab19+c147 (K6907), ab19+c148 (K6908), ab19+c149 (K6909), ab19+c150 (K6910), ab19+c151 (K6911), ab19+c152 (K6912), ab19+c153 (K6913), ab19+c154 (K6914), ab19+c155 (K6915), ab19+c156 (K6916), ab19+c157 (K6917), ab19+c158 (K6918), ab19+c159 (K6919), ab19+c160 (K6920), ab19+c161 (K6921), ab19+c162 (K6922), ab19+c163 (K6923), ab19+c164 (K6924), ab19+c165 (K6925), ab19+c166 (K6926), ab19+c167 (K6927), ab19+c168 (K6928), ab19+c169 (K6929), ab19+c170 (K6930), ab19+c171 (K6931), ab19+c172 (K6932), ab19+c173 (K6933), ab19+c174 (K6934), ab19+c175 (K6935), ab19+c176 (K6936), ab19+c177 (K6937), ab19+c178 (K6938), ab19+c179 (K6939), ab19+c180 (K6940), ab19+c181 (K6941), ab19+c182 (K6942), ab19+c183 (K6943), ab19+c184 (K6944), ab19+c185 (K6945), ab19+c186 (K6946), ab19+c187 (K6947), ab19+c188 (K6948), ab19+c189 (K6949), ab19+c190 (K6950), ab19+c191 (K6951), ab19+c192 (K6952), ab19+c193 (K6953), ab19+c194 (K6954), ab19+c195 (K6955), ab19+c196 (K6956), ab19+c197 (K6957), ab19+c198 (K6958), ab19+c199 (K6959), ab19+c200 (K6960), ab20+c1 (K6961), ab20+c2 (K6962), ab20+c3 (K6963), ab20+c4 (K6964), ab20+c5 (K6965), ab20+c6 (K6966), ab20+c7 (K6967), ab20+c8 (K6968), ab20+c9 (K6969), ab20+c10 (K6970), ab20+c11 (K6971), ab20+c12 (K6972), ab20+c13 (K6973), ab20+c14 (K6974), ab20+c15 (K6975), ab20+c16 (K6976), ab20+c17 (K6977), ab20+c18 (K6978), ab20+c19 (K6979), ab20+c20 (K6980), ab20+c21 (K6981), ab20+c22 (K6982), ab20+c23 (K6983), ab20+c24 (K6984), ab20+c25 (K6985), ab20+c26 (K6986), ab20+c27 (K6987), ab20+c28 (K6988), ab20+c29 (K6989), ab20+c30 (K6990), ab20+c31 (K6991), ab20+c32 (K6992), ab20+c33 (K6993), ab20+c34 (K6994), ab20+c35 (K6995), ab20+c36 (K6996), ab20+c37 (K6997), ab20+c38 (K6998), ab20+c39 (K6999), ab20+c40 (K7000), ab20+c41 (K7001), ab20+c42 (K7002), ab20+c43 (K7003), ab20+c44 (K7004), ab20+c45 (K7005), ab20+c46 (K7006), ab20+c47 (K7007), ab20+c48 (K7008), ab20+c49 (K7009), ab20+c50 (K7010), ab20+c51 (K7011), ab20+c52 (K7012), ab20+c53 (K7013), ab20+c54 (K7014), ab20+c55 (K7015), ab20+c56 (K7016), ab20+c57 (K7017), ab20+c58 (K7018), ab20+c59 (K7019), ab20+c60 (K7020), ab20+c61 (K7021), ab20+c62 (K7022), ab20+c63 (K7023), ab20+c64 (K7024), ab20+c65 (K7025), ab20+c66 (K7026), ab20+c67 (K7027), ab20+c68 (K7028), ab20+c69 (K7029), ab20+c70 (K7030), ab20+c71 (K7031), ab20+c72 (K7032), ab20+c73 (K7033), ab20+c74 (K7034), ab20+c75 (K7035), ab20+c76 (K7036), ab20+c77 (K7037), ab20+c78 (K7038), ab20+c79 (K7039), ab20+c80 (K7040), ab20+c81 (K7041), ab20+c82 (K7042), ab20+c83 (K7043), ab20+c84 (K7044), ab20+c85 (K7045), ab20+c86 (K7046), ab20+c87 (K7047), ab20+c88 (K7048), ab20+c89 (K7049), ab20+c90 (K7050), ab20+c91 (K7051), ab20+c92 (K7052), ab20+c93 (K7053), ab20+c94 (K7054), ab20+c95 (K7055), ab20+c96 (K7056), ab20+c97 (K7057), ab20+c98 (K7058), ab20+c99 (K7059), ab20+c100 (K7060), ab20+c101 (K7061), ab20+c102 (K7062), ab20+c103 (K7063), ab20+c104 (K7064), ab20+c105 (K7065), ab20+c106 (K7066), ab20+c107 (K7067), ab20+c108 (K7068), ab20+c109 (K7069), ab20+c110 (K7070), ab20+c111 (K7071), ab20+c112 (K7072), ab20+c113 (K7073), ab20+c114 (K7074), ab20+c115 (K7075), ab20+c116 (K7076), ab20+c117 (K7077), ab20+c118 (K7078), ab20+c119 (K7079), ab20+c120 (K7080), ab20+c121 (K7081), ab20+c122 (K7082), ab20+c123 (K7083), ab20+c124 (K7084), ab20+c125 (K7085), ab20+c126 (K7086), ab20+c127 (K7087), ab20+c128 (K7088), ab20+c129 (K7089), ab20+c130 (K7090), ab20+c131 (K7091), ab20+c132 (K7092), ab20+c133 (K7093), ab20+c134 (K7094), ab20+c135 (K7095), ab20+c136 (K7096), ab20+c137 (K7097), ab20+c138 (K7098), ab20+c139 (K7099), ab20+c140 (K7100), ab20+c141 (K7101), ab20+c142 (K7102), ab20+c143 (K7103), ab20+c144 (K7104), ab20+c145 (K7105), ab20+c146 (K7106), ab20+c147 (K7107), ab20+c148 (K7108), ab20+c149 (K7109), ab20+c150 (K7110), ab20+c151 (K7111), ab20+c152 (K7112), ab20+c153 (K7113), ab20+c154 (K7114), ab20+c155 (K7115), ab20+c156 (K7116), ab20+c157 (K7117), ab20+c158 (K7118), ab20+c159 (K7119), ab20+c160 (K7120), ab20+c161 (K7121), ab20+c162 (K7122), ab20+c163 (K7123), ab20+c164 (K7124), ab20+c165 (K7125), ab20+c166 (K7126), ab20+c167 (K7127), ab20+c168 (K7128), ab20+c169 (K7129), ab20+c170 (K7130), ab20+c171 (K7131), ab20+c172 (K7132), ab20+c173 (K7133), ab20+c174 (K7134), ab20+c175 (K7135), ab20+c176 (K7136), ab20+c177 (K7137), ab20+c178 (K7138), ab20+c179 (K7139), ab20+c180 (K7140), ab20+c181 (K7141), ab20+c182 (K7142), ab20+c183 (K7143), ab20+c184 (K7144), ab20+c185 (K7145), ab20+c186 (K7146), ab20+c187 (K7147), ab20+c188 (K7148), ab20+c189 (K7149), ab20+c190 (K7150), ab20+c191 (K7151), ab20+c192 (K7152), ab20+c193 (K7153), ab20+c194 (K7154), ab20+c195 (K7155), ab20+c196 (K7156), ab20+c197 (K7157), ab20+c198 (K7158), ab20+c199 (K7159), ab20+c200 (K7160), ab21+c1 (K7161), ab21+c2 (K7162), ab21+c3 (K7163), ab21+c4 (K7164), ab21+c5 (K7165), ab21+c6 (K7166), ab21+c7 (K7167), ab21+c8 (K7168), ab21+c9 (K7169), ab21+c10 (K7170), ab21+c11 (K7171), ab21+c12 (K7172), ab21+c13 (K7173), ab21+c14 (K7174), ab21+c15 (K7175), ab21+c16 (K7176), ab21+c17 (K7177), ab21+c18 (K7178), ab21+c19 (K7179), ab21+c20 (K7180), ab21+c21 (K7181), ab21+c22 (K7182), ab21+c23 (K7183), ab21+c24 (K7184), ab21+c25 (K7185), ab21+c26 (K7186), ab21+c27 (K7187), ab21+c28 (K7188), ab21+c29 (K7189), ab21+c30 (K7190), ab21+c31 (K7191), ab21+c32 (K7192), ab21+c33 (K7193), ab21+c34 (K7194), ab21+c35 (K7195), ab21+c36 (K7196), ab21+c37 (K7197), ab21+c38 (K7198), ab21+c39 (K7199), ab21+c40 (K7200), ab21+c41 (K7201), ab21+c42 (K7202), ab21+c43 (K7203), ab21+c44 (K7204), ab21+c45 (K7205), ab21+c46 (K7206), ab21+c47 (K7207), ab21+c48 (K7208), ab21+c49 (K7209), ab21+c50 (K7210), ab21+c51 (K7211), ab21+c52 (K7212), ab21+c53 (K7213), ab21+c54 (K7214), ab21+c55 (K7215), ab21+c56 (K7216), ab21+c57 (K7217), ab21+c58 (K7218), ab21+c59 (K7219), ab21+c60 (K7220), ab21+c61 (K7221), ab21+c62 (K7222), ab21+c63 (K7223), ab21+c64 (K7224), ab21+c65 (K7225), ab21+c66 (K7226), ab21+c67 (K7227), ab21+c68 (K7228), ab21+c69 (K7229), ab21+c70 (K7230), ab21+c71 (K7231), ab21+c72 (K7232), ab21+c73 (K7233), ab21+c74 (K7234), ab21+c75 (K7235), ab21+c76 (K7236), ab21+c77 (K7237), ab21+c78 (K7238), ab21+c79 (K7239), ab21+c80 (K7240), ab21+c81 (K7241), ab21+c82 (K7242), ab21+c83 (K7243), ab21+c84 (K7244), ab21+c85 (K7245), ab21+c86 (K7246), ab21+c87 (K7247), ab21+c88 (K7248), ab21+c89 (K7249), ab21+c90 (K7250), ab21+c91 (K7251), ab21+c92 (K7252), ab21+c93 (K7253), ab21+c94 (K7254), ab21+c95 (K7255), ab21+c96 (K7256), ab21+c97 (K7257), ab21+c98 (K7258), ab21+c99 (K7259), ab21+c100 (K7260), ab21+c101 (K7261), ab21+c102 (K7262), ab21+c103 (K7263), ab21+c104 (K7264), ab21+c105 (K7265), ab21+c106 (K7266), ab21+c107 (K7267), ab21+c108 (K7268), ab21+c109 (K7269), ab21+c110 (K7270), ab21+c111 (K7271), ab21+c112 (K7272), ab21+c113 (K7273), ab21+c114 (K7274), ab21+c115 (K7275), ab21+c116 (K7276), ab21+c117 (K7277), ab21+c118 (K7278), ab21+c119 (K7279), ab21+c120 (K7280), ab21+c121 (K7281), ab21+c122 (K7282), ab21+c123 (K7283), ab21+c124 (K7284), ab21+c125 (K7285), ab21+c126 (K7286), ab21+c127 (K7287), ab21+c128 (K7288), ab21+c129 (K7289), ab21+c130 (K7290), ab21+c131 (K7291), ab21+c132 (K7292), ab21+c133 (K7293), ab21+c134 (K7294), ab21+c135 (K7295), ab21+c136 (K7296), ab21+c137 (K7297), ab21+c138 (K7298), ab21+c139 (K7299), ab21+c140 (K7300), ab21+c141 (K7301), ab21+c142 (K7302), ab21+c143 (K7303), ab21+c144 (K7304), ab21+c145 (K7305), ab21+c146 (K7306), ab21+c147 (K7307), ab21+c148 (K7308), ab21+c149 (K7309), ab21+c150 (K7310), ab21+c151 (K7311), ab21+c152 (K7312), ab21+c153 (K7313), ab21+c154 (K7314), ab21+c155 (K7315), ab21+c156 (K7316), ab21+c157 (K7317), ab21+c158 (K7318), ab21+c159 (K7319), ab21+c160 (K7320), ab21+c161 (K7321), ab21+c162 (K7322), ab21+c163 (K7323), ab21+c164 (K7324), ab21+c165 (K7325), ab21+c166 (K7326), ab21+c167 (K7327), ab21+c168 (K7328), ab21+c169 (K7329), ab21+c170 (K7330), ab21+c171 (K7331), ab21+c172 (K7332), ab21+c173 (K7333), ab21+c174 (K7334), ab21+c175 (K7335), ab21+c176 (K7336), ab21+c177 (K7337), ab21+c178 (K7338), ab21+c179 (K7339), ab21+c180 (K7340), ab21+c181 (K7341), ab21+c182 (K7342), ab21+c183 (K7343), ab21+c184 (K7344), ab21+c185 (K7345), ab21+c186 (K7346), ab21+c187 (K7347), ab21+c188 (K7348), ab21+c189 (K7349), ab21+c190 (K7350), ab21+c191 (K7351), ab21+c192 (K7352), ab21+c193 (K7353), ab21+c194 (K7354), ab21+c195 (K7355), ab21+c196 (K7356), ab21+c197 (K7357), ab21+c198 (K7358), ab21+c199 (K7359), ab21+c200 (K7360), ab22+c1 (K7361), ab22+c2 (K7362), ab22+c3 (K7363), ab22+c4 (K7364), ab22+c5 (K7365), ab22+c6 (K7366), ab22+c7 (K7367), ab22+c8 (K7368), ab22+c9 (K7369), ab22+c10 (K7370), ab22+c11 (K7371), ab22+c12 (K7372), ab22+c13 (K7373), ab22+c14 (K7374), ab22+c15 (K7375), ab22+c16 (K7376), ab22+c17 (K7377), ab22+c18 (K7378), ab22+c19 (K7379), ab22+c20 (K7380), ab22+c21 (K7381), ab22+c22 (K7382), ab22+c23 (K7383), ab22+c24 (K7384), ab22+c25 (K7385), ab22+c26 (K7386), ab22+c27 (K7387), ab22+c28 (K7388), ab22+c29 (K7389), ab22+c30 (K7390), ab22+c31 (K7391), ab22+c32 (K7392), ab22+c33 (K7393), ab22+c34 (K7394), ab22+c35 (K7395), ab22+c36 (K7396), ab22+c37 (K7397), ab22+c38 (K7398), ab22+c39 (K7399), ab22+c40 (K7400), ab22+c41 (K7401), ab22+c42 (K7402), ab22+c43 (K7403), ab22+c44 (K7404), ab22+c45 (K7405), ab22+c46 (K7406), ab22+c47 (K7407), ab22+c48 (K7408), ab22+c49 (K7409), ab22+c50 (K7410), ab22+c51 (K7411), ab22+c52 (K7412), ab22+c53 (K7413), ab22+c54 (K7414), ab22+c55 (K7415), ab22+c56 (K7416), ab22+c57 (K7417), ab22+c58 (K7418), ab22+c59 (K7419), ab22+c60 (K7420), ab22+c61 (K7421), ab22+c62 (K7422), ab22+c63 (K7423), ab22+c64 (K7424), ab22+c65 (K7425), ab22+c66 (K7426), ab22+c67 (K7427), ab22+c68 (K7428), ab22+c69 (K7429), ab22+c70 (K7430), ab22+c71 (K7431), ab22+c72 (K7432), ab22+c73 (K7433), ab22+c74 (K7434), ab22+c75 (K7435), ab22+c76 (K7436), ab22+c77 (K7437), ab22+c78 (K7438), ab22+c79 (K7439), ab22+c80 (K7440), ab22+c81 (K7441), ab22+c82 (K7442), ab22+c83 (K7443), ab22+c84 (K7444), ab22+c85 (K7445), ab22+c86 (K7446), ab22+c87 (K7447), ab22+c88 (K7448), ab22+c89 (K7449), ab22+c90 (K7450), ab22+c91 (K7451), ab22+c92 (K7452), ab22+c93 (K7453), ab22+c94 (K7454), ab22+c95 (K7455), ab22+c96 (K7456), ab22+c97 (K7457), ab22+c98 (K7458), ab22+c99 (K7459), ab22+c100 (K7460), ab22+c101 (K7461), ab22+c102 (K7462), ab22+c103 (K7463), ab22+c104 (K7464), ab22+c105 (K7465), ab22+c106 (K7466), ab22+c107 (K7467), ab22+c108 (K7468), ab22+c109 (K7469), ab22+c110 (K7470), ab22+c111 (K7471), ab22+c112 (K7472), ab22+c113 (K7473), ab22+c114 (K7474), ab22+c115 (K7475), ab22+c116 (K7476), ab22+c117 (K7477), ab22+c118 (K7478), ab22+c119 (K7479), ab22+c120 (K7480), ab22+c121 (K7481), ab22+c122 (K7482), ab22+c123 (K7483), ab22+c124 (K7484), ab22+c125 (K7485), ab22+c126 (K7486), ab22+c127 (K7487), ab22+c128 (K7488), ab22+c129 (K7489), ab22+c130 (K7490), ab22+c131 (K7491), ab22+c132 (K7492), ab22+c133 (K7493), ab22+c134 (K7494), ab22+c135 (K7495), ab22+c136 (K7496), ab22+c137 (K7497), ab22+c138 (K7498), ab22+c139 (K7499), ab22+c140 (K7500), ab22+c141 (K7501), ab22+c142 (K7502), ab22+c143 (K7503), ab22+c144 (K7504), ab22+c145 (K7505), ab22+c146 (K7506), ab22+c147 (K7507), ab22+c148 (K7508), ab22+c149 (K7509), ab22+c150 (K7510), ab22+c151 (K7511), ab22+c152 (K7512), ab22+c153 (K7513), ab22+c154 (K7514), ab22+c155 (K7515), ab22+c156 (K7516), ab22+c157 (K7517), ab22+c158 (K7518), ab22+c159 (K7519), ab22+c160 (K7520), ab22+c161 (K7521), ab22+c162 (K7522), ab22+c163 (K7523), ab22+c164 (K7524), ab22+c165 (K7525), ab22+c166 (K7526), ab22+c167 (K7527), ab22+c168 (K7528), ab22+c169 (K7529), ab22+c170 (K7530), ab22+c171 (K7531), ab22+c172 (K7532), ab22+c173 (K7533), ab22+c174 (K7534), ab22+c175 (K7535), ab22+c176 (K7536), ab22+c177 (K7537), ab22+c178 (K7538), ab22+c179 (K7539), ab22+c180 (K7540), ab22+c181 (K7541), ab22+c182 (K7542), ab22+c183 (K7543), ab22+c184 (K7544), ab22+c185 (K7545), ab22+c186 (K7546), ab22+c187 (K7547), ab22+c188 (K7548), ab22+c189 (K7549), ab22+c190 (K7550), ab22+c191 (K7551), ab22+c192 (K7552), ab22+c193 (K7553), ab22+c194 (K7554), ab22+c195 (K7555), ab22+c196 (K7556), ab22+c197 (K7557), ab22+c198 (K7558), ab22+c199 (K7559), ab22+c200 (K7560), ab23+c1 (K7561), ab23+c2 (K7562), ab23+c3 (K7563), ab23+c4 (K7564), ab23+c5 (K7565), ab23+c6 (K7566), ab23+c7 (K7567), ab23+c8 (K7568), ab23+c9 (K7569), ab23+c10 (K7570), ab23+c11 (K7571), ab23+c12 (K7572), ab23+c13 (K7573), ab23+c14 (K7574), ab23+c15 (K7575), ab23+c16 (K7576), ab23+c17 (K7577), ab23+c18 (K7578), ab23+c19 (K7579), ab23+c20 (K7580), ab23+c21 (K7581), ab23+c22 (K7582), ab23+c23 (K7583), ab23+c24 (K7584), ab23+c25 (K7585), ab23+c26 (K7586), ab23+c27 (K7587), ab23+c28 (K7588), ab23+c29 (K7589), ab23+c30 (K7590), ab23+c31 (K7591), ab23+c32 (K7592), ab23+c33 (K7593), ab23+c34 (K7594), ab23+c35 (K7595), ab23+c36 (K7596), ab23+c37 (K7597), ab23+c38 (K7598), ab23+c39 (K7599), ab23+c40 (K7600), ab23+c41 (K7601), ab23+c42 (K7602), ab23+c43 (K7603), ab23+c44 (K7604), ab23+c45 (K7605), ab23+c46 (K7606), ab23+c47 (K7607), ab23+c48 (K7608), ab23+c49 (K7609), ab23+c50 (K7610), ab23+c51 (K7611), ab23+c52 (K7612), ab23+c53 (K7613), ab23+c54 (K7614), ab23+c55 (K7615), ab23+c56 (K7616), ab23+c57 (K7617), ab23+c58 (K7618), ab23+c59 (K7619), ab23+c60 (K7620), ab23+c61 (K7621), ab23+c62 (K7622), ab23+c63 (K7623), ab23+c64 (K7624), ab23+c65 (K7625), ab23+c66 (K7626), ab23+c67 (K7627), ab23+c68 (K7628), ab23+c69 (K7629), ab23+c70 (K7630), ab23+c71 (K7631), ab23+c72 (K7632), ab23+c73 (K7633), ab23+c74 (K7634), ab23+c75 (K7635), ab23+c76 (K7636), ab23+c77 (K7637), ab23+c78 (K7638), ab23+c79 (K7639), ab23+c80 (K7640), ab23+c81 (K7641), ab23+c82 (K7642), ab23+c83 (K7643), ab23+c84 (K7644), ab23+c85 (K7645), ab23+c86 (K7646), ab23+c87 (K7647), ab23+c88 (K7648), ab23+c89 (K7649), ab23+c90 (K7650), ab23+c91 (K7651), ab23+c92 (K7652), ab23+c93 (K7653), ab23+c94 (K7654), ab23+c95 (K7655), ab23+c96 (K7656), ab23+c97 (K7657), ab23+c98 (K7658), ab23+c99 (K7659), ab23+c100 (K7660), ab23+c101 (K7661), ab23+c102 (K7662), ab23+c103 (K7663), ab23+c104 (K7664), ab23+c105 (K7665), ab23+c106 (K7666), ab23+c107 (K7667), ab23+c108 (K7668), ab23+c109 (K7669), ab23+c110 (K7670), ab23+c111 (K7671), ab23+c112 (K7672), ab23+c113 (K7673), ab23+c114 (K7674), ab23+c115 (K7675), ab23+c116 (K7676), ab23+c117 (K7677), ab23+c118 (K7678), ab23+c119 (K7679), ab23+c120 (K7680), ab23+c121 (K7681), ab23+c122 (K7682), ab23+c123 (K7683), ab23+c124 (K7684), ab23+c125 (K7685), ab23+c126 (K7686), ab23+c127 (K7687), ab23+c128 (K7688), ab23+c129 (K7689), ab23+c130 (K7690), ab23+c131 (K7691), ab23+c132 (K7692), ab23+c133 (K7693), ab23+c134 (K7694), ab23+c135 (K7695), ab23+c136 (K7696), ab23+c137 (K7697), ab23+c138 (K7698), ab23+c139 (K7699), ab23+c140 (K7700), ab23+c141 (K7701), ab23+c142 (K7702), ab23+c143 (K7703), ab23+c144 (K7704), ab23+c145 (K7705), ab23+c146 (K7706), ab23+c147 (K7707), ab23+c148 (K7708), ab23+c149 (K7709), ab23+c150 (K7710), ab23+c151 (K7711), ab23+c152 (K7712), ab23+c153 (K7713), ab23+c154 (K7714), ab23+c155 (K7715), ab23+c156 (K7716), ab23+c157 (K7717), ab23+c158 (K7718), ab23+c159 (K7719), ab23+c160 (K7720), ab23+c161 (K7721), ab23+c162 (K7722), ab23+c163 (K7723), ab23+c164 (K7724), ab23+c165 (K7725), ab23+c166 (K7726), ab23+c167 (K7727), ab23+c168 (K7728), ab23+c169 (K7729), ab23+c170 (K7730), ab23+c171 (K7731), ab23+c172 (K7732), ab23+c173 (K7733), ab23+c174 (K7734), ab23+c175 (K7735), ab23+c176 (K7736), ab23+c177 (K7737), ab23+c178 (K7738), ab23+c179 (K7739), ab23+c180 (K7740), ab23+c181 (K7741), ab23+c182 (K7742), ab23+c183 (K7743), ab23+c184 (K7744), ab23+c185 (K7745), ab23+c186 (K7746), ab23+c187 (K7747), ab23+c188 (K7748), ab23+c189 (K7749), ab23+c190 (K7750), ab23+c191 (K7751), ab23+c192 (K7752), ab23+c193 (K7753), ab23+c194 (K7754), ab23+c195 (K7755), ab23+c196 (K7756), ab23+c197 (K7757), ab23+c198 (K7758), ab23+c199 (K7759), ab23+c200 (K7760), ab24+c1 (K7761), ab24+c2 (K7762), ab24+c3 (K7763), ab24+c4 (K7764), ab24+c5 (K7765), ab24+c6 (K7766), ab24+c7 (K7767), ab24+c8 (K7768), ab24+c9 (K7769), ab24+c10 (K7770), ab24+c11 (K7771), ab24+c12 (K7772), ab24+c13 (K7773), ab24+c14 (K7774), ab24+c15 (K7775), ab24+c16 (K7776), ab24+c17 (K7777), ab24+c18 (K7778), ab24+c19 (K7779), ab24+c20 (K7780), ab24+c21 (K7781), ab24+c22 (K7782), ab24+c23 (K7783), ab24+c24 (K7784), ab24+c25 (K7785), ab24+c26 (K7786), ab24+c27 (K7787), ab24+c28 (K7788), ab24+c29 (K7789), ab24+c30 (K7790), ab24+c31 (K7791), ab24+c32 (K7792), ab24+c33 (K7793), ab24+c34 (K7794), ab24+c35 (K7795), ab24+c36 (K7796), ab24+c37 (K7797), ab24+c38 (K7798), ab24+c39 (K7799), ab24+c40 (K7800), ab24+c41 (K7801), ab24+c42 (K7802), ab24+c43 (K7803), ab24+c44 (K7804), ab24+c45 (K7805), ab24+c46 (K7806), ab24+c47 (K7807), ab24+c48 (K7808), ab24+c49 (K7809), ab24+c50 (K7810), ab24+c51 (K7811), ab24+c52 (K7812), ab24+c53 (K7813), ab24+c54 (K7814), ab24+c55 (K7815), ab24+c56 (K7816), ab24+c57 (K7817), ab24+c58 (K7818), ab24+c59 (K7819), ab24+c60 (K7820), ab24+c61 (K7821), ab24+c62 (K7822), ab24+c63 (K7823), ab24+c64 (K7824), ab24+c65 (K7825), ab24+c66 (K7826), ab24+c67 (K7827), ab24+c68 (K7828), ab24+c69 (K7829), ab24+c70 (K7830), ab24+c71 (K7831), ab24+c72 (K7832), ab24+c73 (K7833), ab24+c74 (K7834), ab24+c75 (K7835), ab24+c76 (K7836), ab24+c77 (K7837), ab24+c78 (K7838), ab24+c79 (K7839), ab24+c80 (K7840), ab24+c81 (K7841), ab24+c82 (K7842), ab24+c83 (K7843), ab24+c84 (K7844), ab24+c85 (K7845), ab24+c86 (K7846), ab24+c87 (K7847), ab24+c88 (K7848), ab24+c89 (K7849), ab24+c90 (K7850), ab24+c91 (K7851), ab24+c92 (K7852), ab24+c93 (K7853), ab24+c94 (K7854), ab24+c95 (K7855), ab24+c96 (K7856), ab24+c97 (K7857), ab24+c98 (K7858), ab24+c99 (K7859), ab24+c100 (K7860), ab24+c101 (K7861), ab24+c102 (K7862), ab24+c103 (K7863), ab24+c104 (K7864), ab24+c105 (K7865), ab24+c106 (K7866), ab24+c107 (K7867), ab24+c108 (K7868), ab24+c109 (K7869), ab24+c110 (K7870), ab24+c111 (K7871), ab24+c112 (K7872), ab24+c113 (K7873), ab24+c114 (K7874), ab24+c115 (K7875), ab24+c116 (K7876), ab24+c117 (K7877), ab24+c118 (K7878), ab24+c119 (K7879), ab24+c120 (K7880), ab24+c121 (K7881), ab24+c122 (K7882), ab24+c123 (K7883), ab24+c124 (K7884), ab24+c125 (K7885), ab24+c126 (K7886), ab24+c127 (K7887), ab24+c128 (K7888), ab24+c129 (K7889), ab24+c130 (K7890), ab24+c131 (K7891), ab24+c132 (K7892), ab24+c133 (K7893), ab24+c134 (K7894), ab24+c135 (K7895), ab24+c136 (K7896), ab24+c137 (K7897), ab24+c138 (K7898), ab24+c139 (K7899), ab24+c140 (K7900), ab24+c141 (K7901), ab24+c142 (K7902), ab24+c143 (K7903), ab24+c144 (K7904), ab24+c145 (K7905), ab24+c146 (K7906), ab24+c147 (K7907), ab24+c148 (K7908), ab24+c149 (K7909), ab24+c150 (K7910), ab24+c151 (K7911), ab24+c152 (K7912), ab24+c153 (K7913), ab24+c154 (K7914), ab24+c155 (K7915), ab24+c156 (K7916), ab24+c157 (K7917), ab24+c158 (K7918), ab24+c159 (K7919), ab24+c160 (K7920), ab24+c161 (K7921), ab24+c162 (K7922), ab24+c163 (K7923), ab24+c164 (K7924), ab24+c165 (K7925), ab24+c166 (K7926), ab24+c167 (K7927), ab24+c168 (K7928), ab24+c169 (K7929), ab24+c170 (K7930), ab24+c171 (K7931), ab24+c172 (K7932), ab24+c173 (K7933), ab24+c174 (K7934), ab24+c175 (K7935), ab24+c176 (K7936), ab24+c177 (K7937), ab24+c178 (K7938), ab24+c179 (K7939), ab24+c180 (K7940), ab24+c181 (K7941), ab24+c182 (K7942), ab24+c183 (K7943), ab24+c184 (K7944), ab24+c185 (K7945), ab24+c186 (K7946), ab24+c187 (K7947), ab24+c188 (K7948), ab24+c189 (K7949), ab24+c190 (K7950), ab24+c191 (K7951), ab24+c192 (K7952), ab24+c193 (K7953), ab24+c194 (K7954), ab24+c195 (K7955), ab24+c196 (K7956), ab24+c197 (K7957), ab24+c198 (K7958), ab24+c199 (K7959), ab24+c200 (K7960), ab25+c1 (K7961), ab25+c2 (K7962), ab25+c3 (K7963), ab25+c4 (K7964), ab25+c5 (K7965), ab25+c6 (K7966), ab25+c7 (K7967), ab25+c8 (K7968), ab25+c9 (K7969), ab25+c10 (K7970), ab25+c11 (K7971), ab25+c12 (K7972), ab25+c13 (K7973), ab25+c14 (K7974), ab25+c15 (K7975), ab25+c16 (K7976), ab25+c17 (K7977), ab25+c18 (K7978), ab25+c19 (K7979), ab25+c20 (K7980), ab25+c21 (K7981), ab25+c22 (K7982), ab25+c23 (K7983), ab25+c24 (K7984), ab25+c25 (K7985), ab25+c26 (K7986), ab25+c27 (K7987), ab25+c28 (K7988), ab25+c29 (K7989), ab25+c30 (K7990), ab25+c31 (K7991), ab25+c32 (K7992), ab25+c33 (K7993), ab25+c34 (K7994), ab25+c35 (K7995), ab25+c36 (K7996), ab25+c37 (K7997), ab25+c38 (K7998), ab25+c39 (K7999), ab25+c40 (K8000), ab25+c41 (K8001), ab25+c42 (K8002), ab25+c43 (K8003), ab25+c44 (K8004), ab25+c45 (K8005), ab25+c46 (K8006), ab25+c47 (K8007), ab25+c48 (K8008), ab25+c49 (K8009), ab25+c50 (K8010), ab25+c51 (K8011), ab25+c52 (K8012), ab25+c53 (K8013), ab25+c54 (K8014), ab25+c55 (K8015), ab25+c56 (K8016), ab25+c57 (K8017), ab25+c58 (K8018), ab25+c59 (K8019), ab25+c60 (K8020), ab25+c61 (K8021), ab25+c62 (K8022), ab25+c63 (K8023), ab25+c64 (K8024), ab25+c65 (K8025), ab25+c66 (K8026), ab25+c67 (K8027), ab25+c68 (K8028), ab25+c69 (K8029), ab25+c70 (K8030), ab25+c71 (K8031), ab25+c72 (K8032), ab25+c73 (K8033), ab25+c74 (K8034), ab25+c75 (K8035), ab25+c76 (K8036), ab25+c77 (K8037), ab25+c78 (K8038), ab25+c79 (K8039), ab25+c80 (K8040), ab25+c81 (K8041), ab25+c82 (K8042), ab25+c83 (K8043), ab25+c84 (K8044), ab25+c85 (K8045), ab25+c86 (K8046), ab25+c87 (K8047), ab25+c88 (K8048), ab25+c89 (K8049), ab25+c90 (K8050), ab25+c91 (K8051), ab25+c92 (K8052), ab25+c93 (K8053), ab25+c94 (K8054), ab25+c95 (K8055), ab25+c96 (K8056), ab25+c97 (K8057), ab25+c98 (K8058), ab25+c99 (K8059), ab25+c100 (K8060), ab25+c101 (K8061), ab25+c102 (K8062), ab25+c103 (K8063), ab25+c104 (K8064), ab25+c105 (K8065), ab25+c106 (K8066), ab25+c107 (K8067), ab25+c108 (K8068), ab25+c109 (K8069), ab25+c110 (K8070), ab25+c111 (K8071), ab25+c112 (K8072), ab25+c113 (K8073), ab25+c114 (K8074), ab25+c115 (K8075), ab25+c116 (K8076), ab25+c117 (K8077), ab25+c118 (K8078), ab25+c119 (K8079), ab25+c120 (K8080), ab25+c121 (K8081), ab25+c122 (K8082), ab25+c123 (K8083), ab25+c124 (K8084), ab25+c125 (K8085), ab25+c126 (K8086), ab25+c127 (K8087), ab25+c128 (K8088), ab25+c129 (K8089), ab25+c130 (K8090), ab25+c131 (K8091), ab25+c132 (K8092), ab25+c133 (K8093), ab25+c134 (K8094), ab25+c135 (K8095), ab25+c136 (K8096), ab25+c137 (K8097), ab25+c138 (K8098), ab25+c139 (K8099), ab25+c140 (K8100), ab25+c141 (K8101), ab25+c142 (K8102), ab25+c143 (K8103), ab25+c144 (K8104), ab25+c145 (K8105), ab25+c146 (K8106), ab25+c147 (K8107), ab25+c148 (K8108), ab25+c149 (K8109), ab25+c150 (K8110), ab25+c151 (K8111), ab25+c152 (K8112), ab25+c153 (K8113), ab25+c154 (K8114), ab25+c155 (K8115), ab25+c156 (K8116), ab25+c157 (K8117), ab25+c158 (K8118), ab25+c159 (K8119), ab25+c160 (K8120), ab25+c161 (K8121), ab25+c162 (K8122), ab25+c163 (K8123), ab25+c164 (K8124), ab25+c165 (K8125), ab25+c166 (K8126), ab25+c167 (K8127), ab25+c168 (K8128), ab25+c169 (K8129), ab25+c170 (K8130), ab25+c171 (K8131), ab25+c172 (K8132), ab25+c173 (K8133), ab25+c174 (K8134), ab25+c175 (K8135), ab25+c176 (K8136), ab25+c177 (K8137), ab25+c178 (K8138), ab25+c179 (K8139), ab25+c180 (K8140), ab25+c181 (K8141), ab25+c182 (K8142), ab25+c183 (K8143), ab25+c184 (K8144), ab25+c185 (K8145), ab25+c186 (K8146), ab25+c187 (K8147), ab25+c188 (K8148), ab25+c189 (K8149), ab25+c190 (K8150), ab25+c191 (K8151), ab25+c192 (K8152), ab25+c193 (K8153), ab25+c194 (K8154), ab25+c195 (K8155), ab25+c196 (K8156), ab25+c197 (K8157), ab25+c198 (K8158), ab25+c199 (K8159), ab25+c200 (K8160), ab26+c1 (K8161), ab26+c2 (K8162), ab26+c3 (K8163), ab26+c4 (K8164), ab26+c5 (K8165), ab26+c6 (K8166), ab26+c7 (K8167), ab26+c8 (K8168), ab26+c9 (K8169), ab26+c10 (K8170), ab26+c11 (K8171), ab26+c12 (K8172), ab26+c13 (K8173), ab26+c14 (K8174), ab26+c15 (K8175), ab26+c16 (K8176), ab26+c17 (K8177), ab26+c18 (K8178), ab26+c19 (K8179), ab26+c20 (K8180), ab26+c21 (K8181), ab26+c22 (K8182), ab26+c23 (K8183), ab26+c24 (K8184), ab26+c25 (K8185), ab26+c26 (K8186), ab26+c27 (K8187), ab26+c28 (K8188), ab26+c29 (K8189), ab26+c30 (K8190), ab26+c31 (K8191), ab26+c32 (K8192), ab26+c33 (K8193), ab26+c34 (K8194), ab26+c35 (K8195), ab26+c36 (K8196), ab26+c37 (K8197), ab26+c38 (K8198), ab26+c39 (K8199), ab26+c40 (K8200), ab26+c41 (K8201), ab26+c42 (K8202), ab26+c43 (K8203), ab26+c44 (K8204), ab26+c45 (K8205), ab26+c46 (K8206), ab26+c47 (K8207), ab26+c48 (K8208), ab26+c49 (K8209), ab26+c50 (K8210), ab26+c51 (K8211), ab26+c52 (K8212), ab26+c53 (K8213), ab26+c54 (K8214), ab26+c55 (K8215), ab26+c56 (K8216), ab26+c57 (K8217), ab26+c58 (K8218), ab26+c59 (K8219), ab26+c60 (K8220), ab26+c61 (K8221), ab26+c62 (K8222), ab26+c63 (K8223), ab26+c64 (K8224), ab26+c65 (K8225), ab26+c66 (K8226), ab26+c67 (K8227), ab26+c68 (K8228), ab26+c69 (K8229), ab26+c70 (K8230), ab26+c71 (K8231), ab26+c72 (K8232), ab26+c73 (K8233), ab26+c74 (K8234), ab26+c75 (K8235), ab26+c76 (K8236), ab26+c77 (K8237), ab26+c78 (K8238), ab26+c79 (K8239), ab26+c80 (K8240), ab26+c81 (K8241), ab26+c82 (K8242), ab26+c83 (K8243), ab26+c84 (K8244), ab26+c85 (K8245), ab26+c86 (K8246), ab26+c87 (K8247), ab26+c88 (K8248), ab26+c89 (K8249), ab26+c90 (K8250), ab26+c91 (K8251), ab26+c92 (K8252), ab26+c93 (K8253), ab26+c94 (K8254), ab26+c95 (K8255), ab26+c96 (K8256), ab26+c97 (K8257), ab26+c98 (K8258), ab26+c99 (K8259), ab26+c100 (K8260), ab26+c101 (K8261), ab26+c102 (K8262), ab26+c103 (K8263), ab26+c104 (K8264), ab26+c105 (K8265), ab26+c106 (K8266), ab26+c107 (K8267), ab26+c108 (K8268), ab26+c109 (K8269), ab26+c110 (K8270), ab26+c111 (K8271), ab26+c112 (K8272), ab26+c113 (K8273), ab26+c114 (K8274), ab26+c115 (K8275), ab26+c116 (K8276), ab26+c117 (K8277), ab26+c118 (K8278), ab26+c119 (K8279), ab26+c120 (K8280), ab26+c121 (K8281), ab26+c122 (K8282), ab26+c123 (K8283), ab26+c124 (K8284), ab26+c125 (K8285), ab26+c126 (K8286), ab26+c127 (K8287), ab26+c128 (K8288), ab26+c129 (K8289), ab26+c130 (K8290), ab26+c131 (K8291), ab26+c132 (K8292), ab26+c133 (K8293), ab26+c134 (K8294), ab26+c135 (K8295), ab26+c136 (K8296), ab26+c137 (K8297), ab26+c138 (K8298), ab26+c139 (K8299), ab26+c140 (K8300), ab26+c141 (K8301), ab26+c142 (K8302), ab26+c143 (K8303), ab26+c144 (K8304), ab26+c145 (K8305), ab26+c146 (K8306), ab26+c147 (K8307), ab26+c148 (K8308), ab26+c149 (K8309), ab26+c150 (K8310), ab26+c151 (K8311), ab26+c152 (K8312), ab26+c153 (K8313), ab26+c154 (K8314), ab26+c155 (K8315), ab26+c156 (K8316), ab26+c157 (K8317), ab26+c158 (K8318), ab26+c159 (K8319), ab26+c160 (K8320), ab26+c161 (K8321), ab26+c162 (K8322), ab26+c163 (K8323), ab26+c164 (K8324), ab26+c165 (K8325), ab26+c166 (K8326), ab26+c167 (K8327), ab26+c168 (K8328), ab26+c169 (K8329), ab26+c170 (K8330), ab26+c171 (K8331), ab26+c172 (K8332), ab26+c173 (K8333), ab26+c174 (K8334), ab26+c175 (K8335), ab26+c176 (K8336), ab26+c177 (K8337), ab26+c178 (K8338), ab26+c179 (K8339), ab26+c180 (K8340), ab26+c181 (K8341), ab26+c182 (K8342), ab26+c183 (K8343), ab26+c184 (K8344), ab26+c185 (K8345), ab26+c186 (K8346), ab26+c187 (K8347), ab26+c188 (K8348), ab26+c189 (K8349), ab26+c190 (K8350), ab26+c191 (K8351), ab26+c192 (K8352), ab26+c193 (K8353), ab26+c194 (K8354), ab26+c195 (K8355), ab26+c196 (K8356), ab26+c197 (K8357), ab26+c198 (K8358), ab26+c199 (K8359), ab26+c200 (K8360), ab27+c1 (K8361), ab27+c2 (K8362), ab27+c3 (K8363), ab27+c4 (K8364), ab27+c5 (K8365), ab27+c6 (K8366), ab27+c7 (K8367), ab27+c8 (K8368), ab27+c9 (K8369), ab27+c10 (K8370), ab27+c11 (K8371), ab27+c12 (K8372), ab27+c13 (K8373), ab27+c14 (K8374), ab27+c15 (K8375), ab27+c16 (K8376), ab27+c17 (K8377), ab27+c18 (K8378), ab27+c19 (K8379), ab27+c20 (K8380), ab27+c21 (K8381), ab27+c22 (K8382), ab27+c23 (K8383), ab27+c24 (K8384), ab27+c25 (K8385), ab27+c26 (K8386), ab27+c27 (K8387), ab27+c28 (K8388), ab27+c29 (K8389), ab27+c30 (K8390), ab27+c31 (K8391), ab27+c32 (K8392), ab27+c33 (K8393), ab27+c34 (K8394), ab27+c35 (K8395), ab27+c36 (K8396), ab27+c37 (K8397), ab27+c38 (K8398), ab27+c39 (K8399), ab27+c40 (K8400), ab27+c41 (K8401), ab27+c42 (K8402), ab27+c43 (K8403), ab27+c44 (K8404), ab27+c45 (K8405), ab27+c46 (K8406), ab27+c47 (K8407), ab27+c48 (K8408), ab27+c49 (K8409), ab27+c50 (K8410), ab27+c51 (K8411), ab27+c52 (K8412), ab27+c53 (K8413), ab27+c54 (K8414), ab27+c55 (K8415), ab27+c56 (K8416), ab27+c57 (K8417), ab27+c58 (K8418), ab27+c59 (K8419), ab27+c60 (K8420), ab27+c61 (K8421), ab27+c62 (K8422), ab27+c63 (K8423), ab27+c64 (K8424), ab27+c65 (K8425), ab27+c66 (K8426), ab27+c67 (K8427), ab27+c68 (K8428), ab27+c69 (K8429), ab27+c70 (K8430), ab27+c71 (K8431), ab27+c72 (K8432), ab27+c73 (K8433), ab27+c74 (K8434), ab27+c75 (K8435), ab27+c76 (K8436), ab27+c77 (K8437), ab27+c78 (K8438), ab27+c79 (K8439), ab27+c80 (K8440), ab27+c81 (K8441), ab27+c82 (K8442), ab27+c83 (K8443), ab27+c84 (K8444), ab27+c85 (K8445), ab27+c86 (K8446), ab27+c87 (K8447), ab27+c88 (K8448), ab27+c89 (K8449), ab27+c90 (K8450), ab27+c91 (K8451), ab27+c92 (K8452), ab27+c93 (K8453), ab27+c94 (K8454), ab27+c95 (K8455), ab27+c96 (K8456), ab27+c97 (K8457), ab27+c98 (K8458), ab27+c99 (K8459), ab27+c100 (K8460), ab27+c101 (K8461), ab27+c102 (K8462), ab27+c103 (K8463), ab27+c104 (K8464), ab27+c105 (K8465), ab27+c106 (K8466), ab27+c107 (K8467), ab27+c108 (K8468), ab27+c109 (K8469), ab27+c110 (K8470), ab27+c111 (K8471), ab27+c112 (K8472), ab27+c113 (K8473), ab27+c114 (K8474), ab27+c115 (K8475), ab27+c116 (K8476), ab27+c117 (K8477), ab27+c118 (K8478), ab27+c119 (K8479), ab27+c120 (K8480), ab27+c121 (K8481), ab27+c122 (K8482), ab27+c123 (K8483), ab27+c124 (K8484), ab27+c125 (K8485), ab27+c126 (K8486), ab27+c127 (K8487), ab27+c128 (K8488), ab27+c129 (K8489), ab27+c130 (K8490), ab27+c131 (K8491), ab27+c132 (K8492), ab27+c133 (K8493), ab27+c134 (K8494), ab27+c135 (K8495), ab27+c136 (K8496), ab27+c137 (K8497), ab27+c138 (K8498), ab27+c139 (K8499), ab27+c140 (K8500), ab27+c141 (K8501), ab27+c142 (K8502), ab27+c143 (K8503), ab27+c144 (K8504), ab27+c145 (K8505), ab27+c146 (K8506), ab27+c147 (K8507), ab27+c148 (K8508), ab27+c149 (K8509), ab27+c150 (K8510), ab27+c151 (K8511), ab27+c152 (K8512), ab27+c153 (K8513), ab27+c154 (K8514), ab27+c155 (K8515), ab27+c156 (K8516), ab27+c157 (K8517), ab27+c158 (K8518), ab27+c159 (K8519), ab27+c160 (K8520), ab27+c161 (K8521), ab27+c162 (K8522), ab27+c163 (K8523), ab27+c164 (K8524), ab27+c165 (K8525), ab27+c166 (K8526), ab27+c167 (K8527), ab27+c168 (K8528), ab27+c169 (K8529), ab27+c170 (K8530), ab27+c171 (K8531), ab27+c172 (K8532), ab27+c173 (K8533), ab27+c174 (K8534), ab27+c175 (K8535), ab27+c176 (K8536), ab27+c177 (K8537), ab27+c178 (K8538), ab27+c179 (K8539), ab27+c180 (K8540), ab27+c181 (K8541), ab27+c182 (K8542), ab27+c183 (K8543), ab27+c184 (K8544), ab27+c185 (K8545), ab27+c186 (K8546), ab27+c187 (K8547), ab27+c188 (K8548), ab27+c189 (K8549), ab27+c190 (K8550), ab27+c191 (K8551), ab27+c192 (K8552), ab27+c193 (K8553), ab27+c194 (K8554), ab27+c195 (K8555), ab27+c196 (K8556), ab27+c197 (K8557), ab27+c198 (K8558), ab27+c199 (K8559), ab27+c200 (K8560), ab28+c1 (K8561), ab28+c2 (K8562), ab28+c3 (K8563), ab28+c4 (K8564), ab28+c5 (K8565), ab28+c6 (K8566), ab28+c7 (K8567), ab28+c8 (K8568), ab28+c9 (K8569), ab28+c10 (K8570), ab28+c11 (K8571), ab28+c12 (K8572), ab28+c13 (K8573), ab28+c14 (K8574), ab28+c15 (K8575), ab28+c16 (K8576), ab28+c17 (K8577), ab28+c18 (K8578), ab28+c19 (K8579), ab28+c20 (K8580), ab28+c21 (K8581), ab28+c22 (K8582), ab28+c23 (K8583), ab28+c24 (K8584), ab28+c25 (K8585), ab28+c26 (K8586), ab28+c27 (K8587), ab28+c28 (K8588), ab28+c29 (K8589), ab28+c30 (K8590), ab28+c31 (K8591), ab28+c32 (K8592), ab28+c33 (K8593), ab28+c34 (K8594), ab28+c35 (K8595), ab28+c36 (K8596), ab28+c37 (K8597), ab28+c38 (K8598), ab28+c39 (K8599), ab28+c40 (K8600), ab28+c41 (K8601), ab28+c42 (K8602), ab28+c43 (K8603), ab28+c44 (K8604), ab28+c45 (K8605), ab28+c46 (K8606), ab28+c47 (K8607), ab28+c48 (K8608), ab28+c49 (K8609), ab28+c50 (K8610), ab28+c51 (K8611), ab28+c52 (K8612), ab28+c53 (K8613), ab28+c54 (K8614), ab28+c55 (K8615), ab28+c56 (K8616), ab28+c57 (K8617), ab28+c58 (K8618), ab28+c59 (K8619), ab28+c60 (K8620), ab28+c61 (K8621), ab28+c62 (K8622), ab28+c63 (K8623), ab28+c64 (K8624), ab28+c65 (K8625), ab28+c66 (K8626), ab28+c67 (K8627), ab28+c68 (K8628), ab28+c69 (K8629), ab28+c70 (K8630), ab28+c71 (K8631), ab28+c72 (K8632), ab28+c73 (K8633), ab28+c74 (K8634), ab28+c75 (K8635), ab28+c76 (K8636), ab28+c77 (K8637), ab28+c78 (K8638), ab28+c79 (K8639), ab28+c80 (K8640), ab28+c81 (K8641), ab28+c82 (K8642), ab28+c83 (K8643), ab28+c84 (K8644), ab28+c85 (K8645), ab28+c86 (K8646), ab28+c87 (K8647), ab28+c88 (K8648), ab28+c89 (K8649), ab28+c90 (K8650), ab28+c91 (K8651), ab28+c92 (K8652), ab28+c93 (K8653), ab28+c94 (K8654), ab28+c95 (K8655), ab28+c96 (K8656), ab28+c97 (K8657), ab28+c98 (K8658), ab28+c99 (K8659), ab28+c100 (K8660), ab28+c101 (K8661), ab28+c102 (K8662), ab28+c103 (K8663), ab28+c104 (K8664), ab28+c105 (K8665), ab28+c106 (K8666), ab28+c107 (K8667), ab28+c108 (K8668), ab28+c109 (K8669), ab28+c110 (K8670), ab28+c111 (K8671), ab28+c112 (K8672), ab28+c113 (K8673), ab28+c114 (K8674), ab28+c115 (K8675), ab28+c116 (K8676), ab28+c117 (K8677), ab28+c118 (K8678), ab28+c119 (K8679), ab28+c120 (K8680), ab28+c121 (K8681), ab28+c122 (K8682), ab28+c123 (K8683), ab28+c124 (K8684), ab28+c125 (K8685), ab28+c126 (K8686), ab28+c127 (K8687), ab28+c128 (K8688), ab28+c129 (K8689), ab28+c130 (K8690), ab28+c131 (K8691), ab28+c132 (K8692), ab28+c133 (K8693), ab28+c134 (K8694), ab28+c135 (K8695), ab28+c136 (K8696), ab28+c137 (K8697), ab28+c138 (K8698), ab28+c139 (K8699), ab28+c140 (K8700), ab28+c141 (K8701), ab28+c142 (K8702), ab28+c143 (K8703), ab28+c144 (K8704), ab28+c145 (K8705), ab28+c146 (K8706), ab28+c147 (K8707), ab28+c148 (K8708), ab28+c149 (K8709), ab28+c150 (K8710), ab28+c151 (K8711), ab28+c152 (K8712), ab28+c153 (K8713), ab28+c154 (K8714), ab28+c155 (K8715), ab28+c156 (K8716), ab28+c157 (K8717), ab28+c158 (K8718), ab28+c159 (K8719), ab28+c160 (K8720), ab28+c161 (K8721), ab28+c162 (K8722), ab28+c163 (K8723), ab28+c164 (K8724), ab28+c165 (K8725), ab28+c166 (K8726), ab28+c167 (K8727), ab28+c168 (K8728), ab28+c169 (K8729), ab28+c170 (K8730), ab28+c171 (K8731), ab28+c172 (K8732), ab28+c173 (K8733), ab28+c174 (K8734), ab28+c175 (K8735), ab28+c176 (K8736), ab28+c177 (K8737), ab28+c178 (K8738), ab28+c179 (K8739), ab28+c180 (K8740), ab28+c181 (K8741), ab28+c182 (K8742), ab28+c183 (K8743), ab28+c184 (K8744), ab28+c185 (K8745), ab28+c186 (K8746), ab28+c187 (K8747), ab28+c188 (K8748), ab28+c189 (K8749), ab28+c190 (K8750), ab28+c191 (K8751), ab28+c192 (K8752), ab28+c193 (K8753), ab28+c194 (K8754), ab28+c195 (K8755), ab28+c196 (K8756), ab28+c197 (K8757), ab28+c198 (K8758), ab28+c199 (K8759), ab28+c200 (K8760), ab29+c1 (K8761), ab29+c2 (K8762), ab29+c3 (K8763), ab29+c4 (K8764), ab29+c5 (K8765), ab29+c6 (K8766), ab29+c7 (K8767), ab29+c8 (K8768), ab29+c9 (K8769), ab29+c10 (K8770), ab29+c11 (K8771), ab29+c12 (K8772), ab29+c13 (K8773), ab29+c14 (K8774), ab29+c15 (K8775), ab29+c16 (K8776), ab29+c17 (K8777), ab29+c18 (K8778), ab29+c19 (K8779), ab29+c20 (K8780), ab29+c21 (K8781), ab29+c22 (K8782), ab29+c23 (K8783), ab29+c24 (K8784), ab29+c25 (K8785), ab29+c26 (K8786), ab29+c27 (K8787), ab29+c28 (K8788), ab29+c29 (K8789), ab29+c30 (K8790), ab29+c31 (K8791), ab29+c32 (K8792), ab29+c33 (K8793), ab29+c34 (K8794), ab29+c35 (K8795), ab29+c36 (K8796), ab29+c37 (K8797), ab29+c38 (K8798), ab29+c39 (K8799), ab29+c40 (K8800), ab29+c41 (K8801), ab29+c42 (K8802), ab29+c43 (K8803), ab29+c44 (K8804), ab29+c45 (K8805), ab29+c46 (K8806), ab29+c47 (K8807), ab29+c48 (K8808), ab29+c49 (K8809), ab29+c50 (K8810), ab29+c51 (K8811), ab29+c52 (K8812), ab29+c53 (K8813), ab29+c54 (K8814), ab29+c55 (K8815), ab29+c56 (K8816), ab29+c57 (K8817), ab29+c58 (K8818), ab29+c59 (K8819), ab29+c60 (K8820), ab29+c61 (K8821), ab29+c62 (K8822), ab29+c63 (K8823), ab29+c64 (K8824), ab29+c65 (K8825), ab29+c66 (K8826), ab29+c67 (K8827), ab29+c68 (K8828), ab29+c69 (K8829), ab29+c70 (K8830), ab29+c71 (K8831), ab29+c72 (K8832), ab29+c73 (K8833), ab29+c74 (K8834), ab29+c75 (K8835), ab29+c76 (K8836), ab29+c77 (K8837), ab29+c78 (K8838), ab29+c79 (K8839), ab29+c80 (K8840), ab29+c81 (K8841), ab29+c82 (K8842), ab29+c83 (K8843), ab29+c84 (K8844), ab29+c85 (K8845), ab29+c86 (K8846), ab29+c87 (K8847), ab29+c88 (K8848), ab29+c89 (K8849), ab29+c90 (K8850), ab29+c91 (K8851), ab29+c92 (K8852), ab29+c93 (K8853), ab29+c94 (K8854), ab29+c95 (K8855), ab29+c96 (K8856), ab29+c97 (K8857), ab29+c98 (K8858), ab29+c99 (K8859), ab29+c100 (K8860), ab29+c101 (K8861), ab29+c102 (K8862), ab29+c103 (K8863), ab29+c104 (K8864), ab29+c105 (K8865), ab29+c106 (K8866), ab29+c107 (K8867), ab29+c108 (K8868), ab29+c109 (K8869), ab29+c110 (K8870), ab29+c111 (K8871), ab29+c112 (K8872), ab29+c113 (K8873), ab29+c114 (K8874), ab29+c115 (K8875), ab29+c116 (K8876), ab29+c117 (K8877), ab29+c118 (K8878), ab29+c119 (K8879), ab29+c120 (K8880), ab29+c121 (K8881), ab29+c122 (K8882), ab29+c123 (K8883), ab29+c124 (K8884), ab29+c125 (K8885), ab29+c126 (K8886), ab29+c127 (K8887), ab29+c128 (K8888), ab29+c129 (K8889), ab29+c130 (K8890), ab29+c131 (K8891), ab29+c132 (K8892), ab29+c133 (K8893), ab29+c134 (K8894), ab29+c135 (K8895), ab29+c136 (K8896), ab29+c137 (K8897), ab29+c138 (K8898), ab29+c139 (K8899), ab29+c140 (K8900), ab29+c141 (K8901), ab29+c142 (K8902), ab29+c143 (K8903), ab29+c144 (K8904), ab29+c145 (K8905), ab29+c146 (K8906), ab29+c147 (K8907), ab29+c148 (K8908), ab29+c149 (K8909), ab29+c150 (K8910), ab29+c151 (K8911), ab29+c152 (K8912), ab29+c153 (K8913), ab29+c154 (K8914), ab29+c155 (K8915), ab29+c156 (K8916), ab29+c157 (K8917), ab29+c158 (K8918), ab29+c159 (K8919), ab29+c160 (K8920), ab29+c161 (K8921), ab29+c162 (K8922), ab29+c163 (K8923), ab29+c164 (K8924), ab29+c165 (K8925), ab29+c166 (K8926), ab29+c167 (K8927), ab29+c168 (K8928), ab29+c169 (K8929), ab29+c170 (K8930), ab29+c171 (K8931), ab29+c172 (K8932), ab29+c173 (K8933), ab29+c174 (K8934), ab29+c175 (K8935), ab29+c176 (K8936), ab29+c177 (K8937), ab29+c178 (K8938), ab29+c179 (K8939), ab29+c180 (K8940), ab29+c181 (K8941), ab29+c182 (K8942), ab29+c183 (K8943), ab29+c184 (K8944), ab29+c185 (K8945), ab29+c186 (K8946), ab29+c187 (K8947), ab29+c188 (K8948), ab29+c189 (K8949), ab29+c190 (K8950), ab29+c191 (K8951), ab29+c192 (K8952), ab29+c193 (K8953), ab29+c194 (K8954), ab29+c195 (K8955), ab29+c196 (K8956), ab29+c197 (K8957), ab29+c198 (K8958), ab29+c199 (K8959), ab29+c200 (K8960), ab30+c1 (K8961), ab30+c2 (K8962), ab30+c3 (K8963), ab30+c4 (K8964), ab30+c5 (K8965), ab30+c6 (K8966), ab30+c7 (K8967), ab30+c8 (K8968), ab30+c9 (K8969), ab30+c10 (K8970), ab30+c11 (K8971), ab30+c12 (K8972), ab30+c13 (K8973), ab30+c14 (K8974), ab30+c15 (K8975), ab30+c16 (K8976), ab30+c17 (K8977), ab30+c18 (K8978), ab30+c19 (K8979), ab30+c20 (K8980), ab30+c21 (K8981), ab30+c22 (K8982), ab30+c23 (K8983), ab30+c24 (K8984), ab30+c25 (K8985), ab30+c26 (K8986), ab30+c27 (K8987), ab30+c28 (K8988), ab30+c29 (K8989), ab30+c30 (K8990), ab30+c31 (K8991), ab30+c32 (K8992), ab30+c33 (K8993), ab30+c34 (K8994), ab30+c35 (K8995), ab30+c36 (K8996), ab30+c37 (K8997), ab30+c38 (K8998), ab30+c39 (K8999), ab30+c40 (K9000), ab30+c41 (K9001), ab30+c42 (K9002), ab30+c43 (K9003), ab30+c44 (K9004), ab30+c45 (K9005), ab30+c46 (K9006), ab30+c47 (K9007), ab30+c48 (K9008), ab30+c49 (K9009), ab30+c50 (K9010), ab30+c51 (K9011), ab30+c52 (K9012), ab30+c53 (K9013), ab30+c54 (K9014), ab30+c55 (K9015), ab30+c56 (K9016), ab30+c57 (K9017), ab30+c58 (K9018), ab30+c59 (K9019), ab30+c60 (K9020), ab30+c61 (K9021), ab30+c62 (K9022), ab30+c63 (K9023), ab30+c64 (K9024), ab30+c65 (K9025), ab30+c66 (K9026), ab30+c67 (K9027), ab30+c68 (K9028), ab30+c69 (K9029), ab30+c70 (K9030), ab30+c71 (K9031), ab30+c72 (K9032), ab30+c73 (K9033), ab30+c74 (K9034), ab30+c75 (K9035), ab30+c76 (K9036), ab30+c77 (K9037), ab30+c78 (K9038), ab30+c79 (K9039), ab30+c80 (K9040), ab30+c81 (K9041), ab30+c82 (K9042), ab30+c83 (K9043), ab30+c84 (K9044), ab30+c85 (K9045), ab30+c86 (K9046), ab30+c87 (K9047), ab30+c88 (K9048), ab30+c89 (K9049), ab30+c90 (K9050), ab30+c91 (K9051), ab30+c92 (K9052), ab30+c93 (K9053), ab30+c94 (K9054), ab30+c95 (K9055), ab30+c96 (K9056), ab30+c97 (K9057), ab30+c98 (K9058), ab30+c99 (K9059), ab30+c100 (K9060), ab30+c101 (K9061), ab30+c102 (K9062), ab30+c103 (K9063), ab30+c104 (K9064), ab30+c105 (K9065), ab30+c106 (K9066), ab30+c107 (K9067), ab30+c108 (K9068), ab30+c109 (K9069), ab30+c110 (K9070), ab30+c111 (K9071), ab30+c112 (K9072), ab30+c113 (K9073), ab30+c114 (K9074), ab30+c115 (K9075), ab30+c116 (K9076), ab30+c117 (K9077), ab30+c118 (K9078), ab30+c119 (K9079), ab30+c120 (K9080), ab30+c121 (K9081), ab30+c122 (K9082), ab30+c123 (K9083), ab30+c124 (K9084), ab30+c125 (K9085), ab30+c126 (K9086), ab30+c127 (K9087), ab30+c128 (K9088), ab30+c129 (K9089), ab30+c130 (K9090), ab30+c131 (K9091), ab30+c132 (K9092), ab30+c133 (K9093), ab30+c134 (K9094), ab30+c135 (K9095), ab30+c136 (K9096), ab30+c137 (K9097), ab30+c138 (K9098), ab30+c139 (K9099), ab30+c140 (K9100), ab30+c141 (K9101), ab30+c142 (K9102), ab30+c143 (K9103), ab30+c144 (K9104), ab30+c145 (K9105), ab30+c146 (K9106), ab30+c147 (K9107), ab30+c148 (K9108), ab30+c149 (K9109), ab30+c150 (K9110), ab30+c151 (K9111), ab30+c152 (K9112), ab30+c153 (K9113), ab30+c154 (K9114), ab30+c155 (K9115), ab30+c156 (K9116), ab30+c157 (K9117), ab30+c158 (K9118), ab30+c159 (K9119), ab30+c160 (K9120), ab30+c161 (K9121), ab30+c162 (K9122), ab30+c163 (K9123), ab30+c164 (K9124), ab30+c165 (K9125), ab30+c166 (K9126), ab30+c167 (K9127), ab30+c168 (K9128), ab30+c169 (K9129), ab30+c170 (K9130), ab30+c171 (K9131), ab30+c172 (K9132), ab30+c173 (K9133), ab30+c174 (K9134), ab30+c175 (K9135), ab30+c176 (K9136), ab30+c177 (K9137), ab30+c178 (K9138), ab30+c179 (K9139), ab30+c180 (K9140), ab30+c181 (K9141), ab30+c182 (K9142), ab30+c183 (K9143), ab30+c184 (K9144), ab30+c185 (K9145), ab30+c186 (K9146), ab30+c187 (K9147), ab30+c188 (K9148), ab30+c189 (K9149), ab30+c190 (K9150), ab30+c191 (K9151), ab30+c192 (K9152), ab30+c193 (K9153), ab30+c194 (K9154), ab30+c195 (K9155), ab30+c196 (K9156), ab30+c197 (K9157), ab30+c198 (K9158), ab30+c199 (K9159), ab30+c200 (K9160), ab31+c1 (K9161), ab31+c2 (K9162), ab31+c3 (K9163), ab31+c4 (K9164), ab31+c5 (K9165), ab31+c6 (K9166), ab31+c7 (K9167), ab31+c8 (K9168), ab31+c9 (K9169), ab31+c10 (K9170), ab31+c11 (K9171), ab31+c12 (K9172), ab31+c13 (K9173), ab31+c14 (K9174), ab31+c15 (K9175), ab31+c16 (K9176), ab31+c17 (K9177), ab31+c18 (K9178), ab31+c19 (K9179), ab31+c20 (K9180), ab31+c21 (K9181), ab31+c22 (K9182), ab31+c23 (K9183), ab31+c24 (K9184), ab31+c25 (K9185), ab31+c26 (K9186), ab31+c27 (K9187), ab31+c28 (K9188), ab31+c29 (K9189), ab31+c30 (K9190), ab31+c31 (K9191), ab31+c32 (K9192), ab31+c33 (K9193), ab31+c34 (K9194), ab31+c35 (K9195), ab31+c36 (K9196), ab31+c37 (K9197), ab31+c38 (K9198), ab31+c39 (K9199), ab31+c40 (K9200), ab31+c41 (K9201), ab31+c42 (K9202), ab31+c43 (K9203), ab31+c44 (K9204), ab31+c45 (K9205), ab31+c46 (K9206), ab31+c47 (K9207), ab31+c48 (K9208), ab31+c49 (K9209), ab31+c50 (K9210), ab31+c51 (K9211), ab31+c52 (K9212), ab31+c53 (K9213), ab31+c54 (K9214), ab31+c55 (K9215), ab31+c56 (K9216), ab31+c57 (K9217), ab31+c58 (K9218), ab31+c59 (K9219), ab31+c60 (K9220), ab31+c61 (K9221), ab31+c62 (K9222), ab31+c63 (K9223), ab31+c64 (K9224), ab31+c65 (K9225), ab31+c66 (K9226), ab31+c67 (K9227), ab31+c68 (K9228), ab31+c69 (K9229), ab31+c70 (K9230), ab31+c71 (K9231), ab31+c72 (K9232), ab31+c73 (K9233), ab31+c74 (K9234), ab31+c75 (K9235), ab31+c76 (K9236), ab31+c77 (K9237), ab31+c78 (K9238), ab31+c79 (K9239), ab31+c80 (K9240), ab31+c81 (K9241), ab31+c82 (K9242), ab31+c83 (K9243), ab31+c84 (K9244), ab31+c85 (K9245), ab31+c86 (K9246), ab31+c87 (K9247), ab31+c88 (K9248), ab31+c89 (K9249), ab31+c90 (K9250), ab31+c91 (K9251), ab31+c92 (K9252), ab31+c93 (K9253), ab31+c94 (K9254), ab31+c95 (K9255), ab31+c96 (K9256), ab31+c97 (K9257), ab31+c98 (K9258), ab31+c99 (K9259), ab31+c100 (K9260), ab31+c101 (K9261), ab31+c102 (K9262), ab31+c103 (K9263), ab31+c104 (K9264), ab31+c105 (K9265), ab31+c106 (K9266), ab31+c107 (K9267), ab31+c108 (K9268), ab31+c109 (K9269), ab31+c110 (K9270), ab31+c111 (K9271), ab31+c112 (K9272), ab31+c113 (K9273), ab31+c114 (K9274), ab31+c115 (K9275), ab31+c116 (K9276), ab31+c117 (K9277), ab31+c118 (K9278), ab31+c119 (K9279), ab31+c120 (K9280), ab31+c121 (K9281), ab31+c122 (K9282), ab31+c123 (K9283), ab31+c124 (K9284), ab31+c125 (K9285), ab31+c126 (K9286), ab31+c127 (K9287), ab31+c128 (K9288), ab31+c129 (K9289), ab31+c130 (K9290), ab31+c131 (K9291), ab31+c132 (K9292), ab31+c133 (K9293), ab31+c134 (K9294), ab31+c135 (K9295), ab31+c136 (K9296), ab31+c137 (K9297), ab31+c138 (K9298), ab31+c139 (K9299), ab31+c140 (K9300), ab31+c141 (K9301), ab31+c142 (K9302), ab31+c143 (K9303), ab31+c144 (K9304), ab31+c145 (K9305), ab31+c146 (K9306), ab31+c147 (K9307), ab31+c148 (K9308), ab31+c149 (K9309), ab31+c150 (K9310), ab31+c151 (K9311), ab31+c152 (K9312), ab31+c153 (K9313), ab31+c154 (K9314), ab31+c155 (K9315), ab31+c156 (K9316), ab31+c157 (K9317), ab31+c158 (K9318), ab31+c159 (K9319), ab31+c160 (K9320), ab31+c161 (K9321), ab31+c162 (K9322), ab31+c163 (K9323), ab31+c164 (K9324), ab31+c165 (K9325), ab31+c166 (K9326), ab31+c167 (K9327), ab31+c168 (K9328), ab31+c169 (K9329), ab31+c170 (K9330), ab31+c171 (K9331), ab31+c172 (K9332), ab31+c173 (K9333), ab31+c174 (K9334), ab31+c175 (K9335), ab31+c176 (K9336), ab31+c177 (K9337), ab31+c178 (K9338), ab31+c179 (K9339), ab31+c180 (K9340), ab31+c181 (K9341), ab31+c182 (K9342), ab31+c183 (K9343), ab31+c184 (K9344), ab31+c185 (K9345), ab31+c186 (K9346), ab31+c187 (K9347), ab31+c188 (K9348), ab31+c189 (K9349), ab31+c190 (K9350), ab31+c191 (K9351), ab31+c192 (K9352), ab31+c193 (K9353), ab31+c194 (K9354), ab31+c195 (K9355), ab31+c196 (K9356), ab31+c197 (K9357), ab31+c198 (K9358), ab31+c199 (K9359), ab31+c200 (K9360), ab32+c1 (K9361), ab32+c2 (K9362), ab32+c3 (K9363), ab32+c4 (K9364), ab32+c5 (K9365), ab32+c6 (K9366), ab32+c7 (K9367), ab32+c8 (K9368), ab32+c9 (K9369), ab32+c10 (K9370), ab32+c11 (K9371), ab32+c12 (K9372), ab32+c13 (K9373), ab32+c14 (K9374), ab32+c15 (K9375), ab32+c16 (K9376), ab32+c17 (K9377), ab32+c18 (K9378), ab32+c19 (K9379), ab32+c20 (K9380), ab32+c21 (K9381), ab32+c22 (K9382), ab32+c23 (K9383), ab32+c24 (K9384), ab32+c25 (K9385), ab32+c26 (K9386), ab32+c27 (K9387), ab32+c28 (K9388), ab32+c29 (K9389), ab32+c30 (K9390), ab32+c31 (K9391), ab32+c32 (K9392), ab32+c33 (K9393), ab32+c34 (K9394), ab32+c35 (K9395), ab32+c36 (K9396), ab32+c37 (K9397), ab32+c38 (K9398), ab32+c39 (K9399), ab32+c40 (K9400), ab32+c41 (K9401), ab32+c42 (K9402), ab32+c43 (K9403), ab32+c44 (K9404), ab32+c45 (K9405), ab32+c46 (K9406), ab32+c47 (K9407), ab32+c48 (K9408), ab32+c49 (K9409), ab32+c50 (K9410), ab32+c51 (K9411), ab32+c52 (K9412), ab32+c53 (K9413), ab32+c54 (K9414), ab32+c55 (K9415), ab32+c56 (K9416), ab32+c57 (K9417), ab32+c58 (K9418), ab32+c59 (K9419), ab32+c60 (K9420), ab32+c61 (K9421), ab32+c62 (K9422), ab32+c63 (K9423), ab32+c64 (K9424), ab32+c65 (K9425), ab32+c66 (K9426), ab32+c67 (K9427), ab32+c68 (K9428), ab32+c69 (K9429), ab32+c70 (K9430), ab32+c71 (K9431), ab32+c72 (K9432), ab32+c73 (K9433), ab32+c74 (K9434), ab32+c75 (K9435), ab32+c76 (K9436), ab32+c77 (K9437), ab32+c78 (K9438), ab32+c79 (K9439), ab32+c80 (K9440), ab32+c81 (K9441), ab32+c82 (K9442), ab32+c83 (K9443), ab32+c84 (K9444), ab32+c85 (K9445), ab32+c86 (K9446), ab32+c87 (K9447), ab32+c88 (K9448), ab32+c89 (K9449), ab32+c90 (K9450), ab32+c91 (K9451), ab32+c92 (K9452), ab32+c93 (K9453), ab32+c94 (K9454), ab32+c95 (K9455), ab32+c96 (K9456), ab32+c97 (K9457), ab32+c98 (K9458), ab32+c99 (K9459), ab32+c100 (K9460), ab32+c101 (K9461), ab32+c102 (K9462), ab32+c103 (K9463), ab32+c104 (K9464), ab32+c105 (K9465), ab32+c106 (K9466), ab32+c107 (K9467), ab32+c108 (K9468), ab32+c109 (K9469), ab32+c110 (K9470), ab32+c111 (K9471), ab32+c112 (K9472), ab32+c113 (K9473), ab32+c114 (K9474), ab32+c115 (K9475), ab32+c116 (K9476), ab32+c117 (K9477), ab32+c118 (K9478), ab32+c119 (K9479), ab32+c120 (K9480), ab32+c121 (K9481), ab32+c122 (K9482), ab32+c123 (K9483), ab32+c124 (K9484), ab32+c125 (K9485), ab32+c126 (K9486), ab32+c127 (K9487), ab32+c128 (K9488), ab32+c129 (K9489), ab32+c130 (K9490), ab32+c131 (K9491), ab32+c132 (K9492), ab32+c133 (K9493), ab32+c134 (K9494), ab32+c135 (K9495), ab32+c136 (K9496), ab32+c137 (K9497), ab32+c138 (K9498), ab32+c139 (K9499), ab32+c140 (K9500), ab32+c141 (K9501), ab32+c142 (K9502), ab32+c143 (K9503), ab32+c144 (K9504), ab32+c145 (K9505), ab32+c146 (K9506), ab32+c147 (K9507), ab32+c148 (K9508), ab32+c149 (K9509), ab32+c150 (K9510), ab32+c151 (K9511), ab32+c152 (K9512), ab32+c153 (K9513), ab32+c154 (K9514), ab32+c155 (K9515), ab32+c156 (K9516), ab32+c157 (K9517), ab32+c158 (K9518), ab32+c159 (K9519), ab32+c160 (K9520), ab32+c161 (K9521), ab32+c162 (K9522), ab32+c163 (K9523), ab32+c164 (K9524), ab32+c165 (K9525), ab32+c166 (K9526), ab32+c167 (K9527), ab32+c168 (K9528), ab32+c169 (K9529), ab32+c170 (K9530), ab32+c171 (K9531), ab32+c172 (K9532), ab32+c173 (K9533), ab32+c174 (K9534), ab32+c175 (K9535), ab32+c176 (K9536), ab32+c177 (K9537), ab32+c178 (K9538), ab32+c179 (K9539), ab32+c180 (K9540), ab32+c181 (K9541), ab32+c182 (K9542), ab32+c183 (K9543), ab32+c184 (K9544), ab32+c185 (K9545), ab32+c186 (K9546), ab32+c187 (K9547), ab32+c188 (K9548), ab32+c189 (K9549), ab32+c190 (K9550), ab32+c191 (K9551), ab32+c192 (K9552), ab32+c193 (K9553), ab32+c194 (K9554), ab32+c195 (K9555), ab32+c196 (K9556), ab32+c197 (K9557), ab32+c198 (K9558), ab32+c199 (K9559), ab32+c200 (K9560), ab33+c1 (K9561), ab33+c2 (K9562), ab33+c3 (K9563), ab33+c4 (K9564), ab33+c5 (K9565), ab33+c6 (K9566), ab33+c7 (K9567), ab33+c8 (K9568), ab33+c9 (K9569), ab33+c10 (K9570), ab33+c11 (K9571), ab33+c12 (K9572), ab33+c13 (K9573), ab33+c14 (K9574), ab33+c15 (K9575), ab33+c16 (K9576), ab33+c17 (K9577), ab33+c18 (K9578), ab33+c19 (K9579), ab33+c20 (K9580), ab33+c21 (K9581), ab33+c22 (K9582), ab33+c23 (K9583), ab33+c24 (K9584), ab33+c25 (K9585), ab33+c26 (K9586), ab33+c27 (K9587), ab33+c28 (K9588), ab33+c29 (K9589), ab33+c30 (K9590), ab33+c31 (K9591), ab33+c32 (K9592), ab33+c33 (K9593), ab33+c34 (K9594), ab33+c35 (K9595), ab33+c36 (K9596), ab33+c37 (K9597), ab33+c38 (K9598), ab33+c39 (K9599), ab33+c40 (K9600), ab33+c41 (K9601), ab33+c42 (K9602), ab33+c43 (K9603), ab33+c44 (K9604), ab33+c45 (K9605), ab33+c46 (K9606), ab33+c47 (K9607), ab33+c48 (K9608), ab33+c49 (K9609), ab33+c50 (K9610), ab33+c51 (K9611), ab33+c52 (K9612), ab33+c53 (K9613), ab33+c54 (K9614), ab33+c55 (K9615), ab33+c56 (K9616), ab33+c57 (K9617), ab33+c58 (K9618), ab33+c59 (K9619), ab33+c60 (K9620), ab33+c61 (K9621), ab33+c62 (K9622), ab33+c63 (K9623), ab33+c64 (K9624), ab33+c65 (K9625), ab33+c66 (K9626), ab33+c67 (K9627), ab33+c68 (K9628), ab33+c69 (K9629), ab33+c70 (K9630), ab33+c71 (K9631), ab33+c72 (K9632), ab33+c73 (K9633), ab33+c74 (K9634), ab33+c75 (K9635), ab33+c76 (K9636), ab33+c77 (K9637), ab33+c78 (K9638), ab33+c79 (K9639), ab33+c80 (K9640), ab33+c81 (K9641), ab33+c82 (K9642), ab33+c83 (K9643), ab33+c84 (K9644), ab33+c85 (K9645), ab33+c86 (K9646), ab33+c87 (K9647), ab33+c88 (K9648), ab33+c89 (K9649), ab33+c90 (K9650), ab33+c91 (K9651), ab33+c92 (K9652), ab33+c93 (K9653), ab33+c94 (K9654), ab33+c95 (K9655), ab33+c96 (K9656), ab33+c97 (K9657), ab33+c98 (K9658), ab33+c99 (K9659), ab33+c100 (K9660), ab33+c101 (K9661), ab33+c102 (K9662), ab33+c103 (K9663), ab33+c104 (K9664), ab33+c105 (K9665), ab33+c106 (K9666), ab33+c107 (K9667), ab33+c108 (K9668), ab33+c109 (K9669), ab33+c110 (K9670), ab33+c111 (K9671), ab33+c112 (K9672), ab33+c113 (K9673), ab33+c114 (K9674), ab33+c115 (K9675), ab33+c116 (K9676), ab33+c117 (K9677), ab33+c118 (K9678), ab33+c119 (K9679), ab33+c120 (K9680), ab33+c121 (K9681), ab33+c122 (K9682), ab33+c123 (K9683), ab33+c124 (K9684), ab33+c125 (K9685), ab33+c126 (K9686), ab33+c127 (K9687), ab33+c128 (K9688), ab33+c129 (K9689), ab33+c130 (K9690), ab33+c131 (K9691), ab33+c132 (K9692), ab33+c133 (K9693), ab33+c134 (K9694), ab33+c135 (K9695), ab33+c136 (K9696), ab33+c137 (K9697), ab33+c138 (K9698), ab33+c139 (K9699), ab33+c140 (K9700), ab33+c141 (K9701), ab33+c142 (K9702), ab33+c143 (K9703), ab33+c144 (K9704), ab33+c145 (K9705), ab33+c146 (K9706), ab33+c147 (K9707), ab33+c148 (K9708), ab33+c149 (K9709), ab33+c150 (K9710), ab33+c151 (K9711), ab33+c152 (K9712), ab33+c153 (K9713), ab33+c154 (K9714), ab33+c155 (K9715), ab33+c156 (K9716), ab33+c157 (K9717), ab33+c158 (K9718), ab33+c159 (K9719), ab33+c160 (K9720), ab33+c161 (K9721), ab33+c162 (K9722), ab33+c163 (K9723), ab33+c164 (K9724), ab33+c165 (K9725), ab33+c166 (K9726), ab33+c167 (K9727), ab33+c168 (K9728), ab33+c169 (K9729), ab33+c170 (K9730), ab33+c171 (K9731), ab33+c172 (K9732), ab33+c173 (K9733), ab33+c174 (K9734), ab33+c175 (K9735), ab33+c176 (K9736), ab33+c177 (K9737), ab33+c178 (K9738), ab33+c179 (K9739), ab33+c180 (K9740), ab33+c181 (K9741), ab33+c182 (K9742), ab33+c183 (K9743), ab33+c184 (K9744), ab33+c185 (K9745), ab33+c186 (K9746), ab33+c187 (K9747), ab33+c188 (K9748), ab33+c189 (K9749), ab33+c190 (K9750), ab33+c191 (K9751), ab33+c192 (K9752), ab33+c193 (K9753), ab33+c194 (K9754), ab33+c195 (K9755), ab33+c196 (K9756), ab33+c197 (K9757), ab33+c198 (K9758), ab33+c199 (K9759), ab33+c200 (K9760), ab34+c1 (K9761), ab34+c2 (K9762), ab34+c3 (K9763), ab34+c4 (K9764), ab34+c5 (K9765), ab34+c6 (K9766), ab34+c7 (K9767), ab34+c8 (K9768), ab34+c9 (K9769), ab34+c10 (K9770), ab34+c11 (K9771), ab34+c12 (K9772), ab34+c13 (K9773), ab34+c14 (K9774), ab34+c15 (K9775), ab34+c16 (K9776), ab34+c17 (K9777), ab34+c18 (K9778), ab34+c19 (K9779), ab34+c20 (K9780), ab34+c21 (K9781), ab34+c22 (K9782), ab34+c23 (K9783), ab34+c24 (K9784), ab34+c25 (K9785), ab34+c26 (K9786), ab34+c27 (K9787), ab34+c28 (K9788), ab34+c29 (K9789), ab34+c30 (K9790), ab34+c31 (K9791), ab34+c32 (K9792), ab34+c33 (K9793), ab34+c34 (K9794), ab34+c35 (K9795), ab34+c36 (K9796), ab34+c37 (K9797), ab34+c38 (K9798), ab34+c39 (K9799), ab34+c40 (K9800), ab34+c41 (K9801), ab34+c42 (K9802), ab34+c43 (K9803), ab34+c44 (K9804), ab34+c45 (K9805), ab34+c46 (K9806), ab34+c47 (K9807), ab34+c48 (K9808), ab34+c49 (K9809), ab34+c50 (K9810), ab34+c51 (K9811), ab34+c52 (K9812), ab34+c53 (K9813), ab34+c54 (K9814), ab34+c55 (K9815), ab34+c56 (K9816), ab34+c57 (K9817), ab34+c58 (K9818), ab34+c59 (K9819), ab34+c60 (K9820), ab34+c61 (K9821), ab34+c62 (K9822), ab34+c63 (K9823), ab34+c64 (K9824), ab34+c65 (K9825), ab34+c66 (K9826), ab34+c67 (K9827), ab34+c68 (K9828), ab34+c69 (K9829), ab34+c70 (K9830), ab34+c71 (K9831), ab34+c72 (K9832), ab34+c73 (K9833), ab34+c74 (K9834), ab34+c75 (K9835), ab34+c76 (K9836), ab34+c77 (K9837), ab34+c78 (K9838), ab34+c79 (K9839), ab34+c80 (K9840), ab34+c81 (K9841), ab34+c82 (K9842), ab34+c83 (K9843), ab34+c84 (K9844), ab34+c85 (K9845), ab34+c86 (K9846), ab34+c87 (K9847), ab34+c88 (K9848), ab34+c89 (K9849), ab34+c90 (K9850), ab34+c91 (K9851), ab34+c92 (K9852), ab34+c93 (K9853), ab34+c94 (K9854), ab34+c95 (K9855), ab34+c96 (K9856), ab34+c97 (K9857), ab34+c98 (K9858), ab34+c99 (K9859), ab34+c100 (K9860), ab34+c101 (K9861), ab34+c102 (K9862), ab34+c103 (K9863), ab34+c104 (K9864), ab34+c105 (K9865), ab34+c106 (K9866), ab34+c107 (K9867), ab34+c108 (K9868), ab34+c109 (K9869), ab34+c110 (K9870), ab34+c111 (K9871), ab34+c112 (K9872), ab34+c113 (K9873), ab34+c114 (K9874), ab34+c115 (K9875), ab34+c116 (K9876), ab34+c117 (K9877), ab34+c118 (K9878), ab34+c119 (K9879), ab34+c120 (K9880), ab34+c121 (K9881), ab34+c122 (K9882), ab34+c123 (K9883), ab34+c124 (K9884), ab34+c125 (K9885), ab34+c126 (K9886), ab34+c127 (K9887), ab34+c128 (K9888), ab34+c129 (K9889), ab34+c130 (K9890), ab34+c131 (K9891), ab34+c132 (K9892), ab34+c133 (K9893), ab34+c134 (K9894), ab34+c135 (K9895), ab34+c136 (K9896), ab34+c137 (K9897), ab34+c138 (K9898), ab34+c139 (K9899), ab34+c140 (K9900), ab34+c141 (K9901), ab34+c142 (K9902), ab34+c143 (K9903), ab34+c144 (K9904), ab34+c145 (K9905), ab34+c146 (K9906), ab34+c147 (K9907), ab34+c148 (K9908), ab34+c149 (K9909), ab34+c150 (K9910), ab34+c151 (K9911), ab34+c152 (K9912), ab34+c153 (K9913), ab34+c154 (K9914), ab34+c155 (K9915), ab34+c156 (K9916), ab34+c157 (K9917), ab34+c158 (K9918), ab34+c159 (K9919), ab34+c160 (K9920), ab34+c161 (K9921), ab34+c162 (K9922), ab34+c163 (K9923), ab34+c164 (K9924), ab34+c165 (K9925), ab34+c166 (K9926), ab34+c167 (K9927), ab34+c168 (K9928), ab34+c169 (K9929), ab34+c170 (K9930), ab34+c171 (K9931), ab34+c172 (K9932), ab34+c173 (K9933), ab34+c174 (K9934), ab34+c175 (K9935), ab34+c176 (K9936), ab34+c177 (K9937), ab34+c178 (K9938), ab34+c179 (K9939), ab34+c180 (K9940), ab34+c181 (K9941), ab34+c182 (K9942), ab34+c183 (K9943), ab34+c184 (K9944), ab34+c185 (K9945), ab34+c186 (K9946), ab34+c187 (K9947), ab34+c188 (K9948), ab34+c189 (K9949), ab34+c190 (K9950), ab34+c191 (K9951), ab34+c192 (K9952), ab34+c193 (K9953), ab34+c194 (K9954), ab34+c195 (K9955), ab34+c196 (K9956), ab34+c197 (K9957), ab34+c198 (K9958), ab34+c199 (K9959), ab34+c200 (K9960), ab35+c1 (K9961), ab35+c2 (K9962), ab35+c3 (K9963), ab35+c4 (K9964), ab35+c5 (K9965), ab35+c6 (K9966), ab35+c7 (K9967), ab35+c8 (K9968), ab35+c9 (K9969), ab35+c10 (K9970), ab35+c11 (K9971), ab35+c12 (K9972), ab35+c13 (K9973), ab35+c14 (K9974), ab35+c15 (K9975), ab35+c16 (K9976), ab35+c17 (K9977), ab35+c18 (K9978), ab35+c19 (K9979), ab35+c20 (K9980), ab35+c21 (K9981), ab35+c22 (K9982), ab35+c23 (K9983), ab35+c24 (K9984), ab35+c25 (K9985), ab35+c26 (K9986), ab35+c27 (K9987), ab35+c28 (K9988), ab35+c29 (K9989), ab35+c30 (K9990), ab35+c31 (K9991), ab35+c32 (K9992), ab35+c33 (K9993), ab35+c34 (K9994), ab35+c35 (K9995), ab35+c36 (K9996), ab35+c37 (K9997), ab35+c38 (K9998), ab35+c39 (K9999), ab35+c40 (K10000), ab35+c41 (K10001), ab35+c42 (K10002), ab35+c43 (K10003), ab35+c44 (K10004), ab35+c45 (K10005), ab35+c46 (K10006), ab35+c47 (K10007), ab35+c48 (K10008), ab35+c49 (K10009), ab35+c50 (K10010), ab35+c51 (K10011), ab35+c52 (K10012), ab35+c53 (K10013), ab35+c54 (K10014), ab35+c55 (K10015), ab35+c56 (K10016), ab35+c57 (K10017), ab35+c58 (K10018), ab35+c59 (K10019), ab35+c60 (K10020), ab35+c61 (K10021), ab35+c62 (K10022), ab35+c63 (K10023), ab35+c64 (K10024), ab35+c65 (K10025), ab35+c66 (K10026), ab35+c67 (K10027), ab35+c68 (K10028), ab35+c69 (K10029), ab35+c70 (K10030), ab35+c71 (K10031), ab35+c72 (K10032), ab35+c73 (K10033), ab35+c74 (K10034), ab35+c75 (K10035), ab35+c76 (K10036), ab35+c77 (K10037), ab35+c78 (K10038), ab35+c79 (K10039), ab35+c80 (K10040), ab35+c81 (K10041), ab35+c82 (K10042), ab35+c83 (K10043), ab35+c84 (K10044), ab35+c85 (K10045), ab35+c86 (K10046), ab35+c87 (K10047), ab35+c88 (K10048), ab35+c89 (K10049), ab35+c90 (K10050), ab35+c91 (K10051), ab35+c92 (K10052), ab35+c93 (K10053), ab35+c94 (K10054), ab35+c95 (K10055), ab35+c96 (K10056), ab35+c97 (K10057), ab35+c98 (K10058), ab35+c99 (K10059), ab35+c100 (K10060), ab35+c101 (K10061), ab35+c102 (K10062), ab35+c103 (K10063), ab35+c104 (K10064), ab35+c105 (K10065), ab35+c106 (K10066), ab35+c107 (K10067), ab35+c108 (K10068), ab35+c109 (K10069), ab35+c110 (K10070), ab35+c111 (K10071), ab35+c112 (K10072), ab35+c113 (K10073), ab35+c114 (K10074), ab35+c115 (K10075), ab35+c116 (K10076), ab35+c117 (K10077), ab35+c118 (K10078), ab35+c119 (K10079), ab35+c120 (K10080), ab35+c121 (K10081), ab35+c122 (K10082), ab35+c123 (K10083), ab35+c124 (K10084), ab35+c125 (K10085), ab35+c126 (K10086), ab35+c127 (K10087), ab35+c128 (K10088), ab35+c129 (K10089), ab35+c130 (K10090), ab35+c131 (K10091), ab35+c132 (K10092), ab35+c133 (K10093), ab35+c134 (K10094), ab35+c135 (K10095), ab35+c136 (K10096), ab35+c137 (K10097), ab35+c138 (K10098), ab35+c139 (K10099), ab35+c140 (K10100), ab35+c141 (K10101), ab35+c142 (K10102), ab35+c143 (K10103), ab35+c144 (K10104), ab35+c145 (K10105), ab35+c146 (K10106), ab35+c147 (K10107), ab35+c148 (K10108), ab35+c149 (K10109), ab35+c150 (K10110), ab35+c151 (K10111), ab35+c152 (K10112), ab35+c153 (K10113), ab35+c154 (K10114), ab35+c155 (K10115), ab35+c156 (K10116), ab35+c157 (K10117), ab35+c158 (K10118), ab35+c159 (K10119), ab35+c160 (K10120), ab35+c161 (K10121), ab35+c162 (K10122), ab35+c163 (K10123), ab35+c164 (K10124), ab35+c165 (K10125), ab35+c166 (K10126), ab35+c167 (K10127), ab35+c168 (K10128), ab35+c169 (K10129), ab35+c170 (K10130), ab35+c171 (K10131), ab35+c172 (K10132), ab35+c173 (K10133), ab35+c174 (K10134), ab35+c175 (K10135), ab35+c176 (K10136), ab35+c177 (K10137), ab35+c178 (K10138), ab35+c179 (K10139), ab35+c180 (K10140), ab35+c181 (K10141), ab35+c182 (K10142), ab35+c183 (K10143), ab35+c184 (K10144), ab35+c185 (K10145), ab35+c186 (K10146), ab35+c187 (K10147), ab35+c188 (K10148), ab35+c189 (K10149), ab35+c190 (K10150), ab35+c191 (K10151), ab35+c192 (K10152), ab35+c193 (K10153), ab35+c194 (K10154), ab35+c195 (K10155), ab35+c196 (K10156), ab35+c197 (K10157), ab35+c198 (K10158), ab35+c199 (K10159), ab35+c200 (K10160), ab36+c1 (K10161), ab36+c2 (K10162), ab36+c3 (K10163), ab36+c4 (K10164), ab36+c5 (K10165), ab36+c6 (K10166), ab36+c7 (K10167), ab36+c8 (K10168), ab36+c9 (K10169), ab36+c10 (K10170), ab36+c11 (K10171), ab36+c12 (K10172), ab36+c13 (K10173), ab36+c14 (K10174), ab36+c15 (K10175), ab36+c16 (K10176), ab36+c17 (K10177), ab36+c18 (K10178), ab36+c19 (K10179), ab36+c20 (K10180), ab36+c21 (K10181), ab36+c22 (K10182), ab36+c23 (K10183), ab36+c24 (K10184), ab36+c25 (K10185), ab36+c26 (K10186), ab36+c27 (K10187), ab36+c28 (K10188), ab36+c29 (K10189), ab36+c30 (K10190), ab36+c31 (K10191), ab36+c32 (K10192), ab36+c33 (K10193), ab36+c34 (K10194), ab36+c35 (K10195), ab36+c36 (K10196), ab36+c37 (K10197), ab36+c38 (K10198), ab36+c39 (K10199), ab36+c40 (K10200), ab36+c41 (K10201), ab36+c42 (K10202), ab36+c43 (K10203), ab36+c44 (K10204), ab36+c45 (K10205), ab36+c46 (K10206), ab36+c47 (K10207), ab36+c48 (K10208), ab36+c49 (K10209), ab36+c50 (K10210), ab36+c51 (K10211), ab36+c52 (K10212), ab36+c53 (K10213), ab36+c54 (K10214), ab36+c55 (K10215), ab36+c56 (K10216), ab36+c57 (K10217), ab36+c58 (K10218), ab36+c59 (K10219), ab36+c60 (K10220), ab36+c61 (K10221), ab36+c62 (K10222), ab36+c63 (K10223), ab36+c64 (K10224), ab36+c65 (K10225), ab36+c66 (K10226), ab36+c67 (K10227), ab36+c68 (K10228), ab36+c69 (K10229), ab36+c70 (K10230), ab36+c71 (K10231), ab36+c72 (K10232), ab36+c73 (K10233), ab36+c74 (K10234), ab36+c75 (K10235), ab36+c76 (K10236), ab36+c77 (K10237), ab36+c78 (K10238), ab36+c79 (K10239), ab36+c80 (K10240), ab36+c81 (K10241), ab36+c82 (K10242), ab36+c83 (K10243), ab36+c84 (K10244), ab36+c85 (K10245), ab36+c86 (K10246), ab36+c87 (K10247), ab36+c88 (K10248), ab36+c89 (K10249), ab36+c90 (K10250), ab36+c91 (K10251), ab36+c92 (K10252), ab36+c93 (K10253), ab36+c94 (K10254), ab36+c95 (K10255), ab36+c96 (K10256), ab36+c97 (K10257), ab36+c98 (K10258), ab36+c99 (K10259), ab36+c100 (K10260), ab36+c101 (K10261), ab36+c102 (K10262), ab36+c103 (K10263), ab36+c104 (K10264), ab36+c105 (K10265), ab36+c106 (K10266), ab36+c107 (K10267), ab36+c108 (K10268), ab36+c109 (K10269), ab36+c110 (K10270), ab36+c111 (K10271), ab36+c112 (K10272), ab36+c113 (K10273), ab36+c114 (K10274), ab36+c115 (K10275), ab36+c116 (K10276), ab36+c117 (K10277), ab36+c118 (K10278), ab36+c119 (K10279), ab36+c120 (K10280), ab36+c121 (K10281), ab36+c122 (K10282), ab36+c123 (K10283), ab36+c124 (K10284), ab36+c125 (K10285), ab36+c126 (K10286), ab36+c127 (K10287), ab36+c128 (K10288), ab36+c129 (K10289), ab36+c130 (K10290), ab36+c131 (K10291), ab36+c132 (K10292), ab36+c133 (K10293), ab36+c134 (K10294), ab36+c135 (K10295), ab36+c136 (K10296), ab36+c137 (K10297), ab36+c138 (K10298), ab36+c139 (K10299), ab36+c140 (K10300), ab36+c141 (K10301), ab36+c142 (K10302), ab36+c143 (K10303), ab36+c144 (K10304), ab36+c145 (K10305), ab36+c146 (K10306), ab36+c147 (K10307), ab36+c148 (K10308), ab36+c149 (K10309), ab36+c150 (K10310), ab36+c151 (K10311), ab36+c152 (K10312), ab36+c153 (K10313), ab36+c154 (K10314), ab36+c155 (K10315), ab36+c156 (K10316), ab36+c157 (K10317), ab36+c158 (K10318), ab36+c159 (K10319), ab36+c160 (K10320), ab36+c161 (K10321), ab36+c162 (K10322), ab36+c163 (K10323), ab36+c164 (K10324), ab36+c165 (K10325), ab36+c166 (K10326), ab36+c167 (K10327), ab36+c168 (K10328), ab36+c169 (K10329), ab36+c170 (K10330), ab36+c171 (K10331), ab36+c172 (K10332), ab36+c173 (K10333), ab36+c174 (K10334), ab36+c175 (K10335), ab36+c176 (K10336), ab36+c177 (K10337), ab36+c178 (K10338), ab36+c179 (K10339), ab36+c180 (K10340), ab36+c181 (K10341), ab36+c182 (K10342), ab36+c183 (K10343), ab36+c184 (K10344), ab36+c185 (K10345), ab36+c186 (K10346), ab36+c187 (K10347), ab36+c188 (K10348), ab36+c189 (K10349), ab36+c190 (K10350), ab36+c191 (K10351), ab36+c192 (K10352), ab36+c193 (K10353), ab36+c194 (K10354), ab36+c195 (K10355), ab36+c196 (K10356), ab36+c197 (K10357), ab36+c198 (K10358), ab36+c199 (K10359), ab36+c200 (K10360), ab37+c1 (K10361), ab37+c2 (K10362), ab37+c3 (K10363), ab37+c4 (K10364), ab37+c5 (K10365), ab37+c6 (K10366), ab37+c7 (K10367), ab37+c8 (K10368), ab37+c9 (K10369), ab37+c10 (K10370), ab37+c11 (K10371), ab37+c12 (K10372), ab37+c13 (K10373), ab37+c14 (K10374), ab37+c15 (K10375), ab37+c16 (K10376), ab37+c17 (K10377), ab37+c18 (K10378), ab37+c19 (K10379), ab37+c20 (K10380), ab37+c21 (K10381), ab37+c22 (K10382), ab37+c23 (K10383), ab37+c24 (K10384), ab37+c25 (K10385), ab37+c26 (K10386), ab37+c27 (K10387), ab37+c28 (K10388), ab37+c29 (K10389), ab37+c30 (K10390), ab37+c31 (K10391), ab37+c32 (K10392), ab37+c33 (K10393), ab37+c34 (K10394), ab37+c35 (K10395), ab37+c36 (K10396), ab37+c37 (K10397), ab37+c38 (K10398), ab37+c39 (K10399), ab37+c40 (K10400), ab37+c41 (K10401), ab37+c42 (K10402), ab37+c43 (K10403), ab37+c44 (K10404), ab37+c45 (K10405), ab37+c46 (K10406), ab37+c47 (K10407), ab37+c48 (K10408), ab37+c49 (K10409), ab37+c50 (K10410), ab37+c51 (K10411), ab37+c52 (K10412), ab37+c53 (K10413), ab37+c54 (K10414), ab37+c55 (K10415), ab37+c56 (K10416), ab37+c57 (K10417), ab37+c58 (K10418), ab37+c59 (K10419), ab37+c60 (K10420), ab37+c61 (K10421), ab37+c62 (K10422), ab37+c63 (K10423), ab37+c64 (K10424), ab37+c65 (K10425), ab37+c66 (K10426), ab37+c67 (K10427), ab37+c68 (K10428), ab37+c69 (K10429), ab37+c70 (K10430), ab37+c71 (K10431), ab37+c72 (K10432), ab37+c73 (K10433), ab37+c74 (K10434), ab37+c75 (K10435), ab37+c76 (K10436), ab37+c77 (K10437), ab37+c78 (K10438), ab37+c79 (K10439), ab37+c80 (K10440), ab37+c81 (K10441), ab37+c82 (K10442), ab37+c83 (K10443), ab37+c84 (K10444), ab37+c85 (K10445), ab37+c86 (K10446), ab37+c87 (K10447), ab37+c88 (K10448), ab37+c89 (K10449), ab37+c90 (K10450), ab37+c91 (K10451), ab37+c92 (K10452), ab37+c93 (K10453), ab37+c94 (K10454), ab37+c95 (K10455), ab37+c96 (K10456), ab37+c97 (K10457), ab37+c98 (K10458), ab37+c99 (K10459), ab37+c100 (K10460), ab37+c101 (K10461), ab37+c102 (K10462), ab37+c103 (K10463), ab37+c104 (K10464), ab37+c105 (K10465), ab37+c106 (K10466), ab37+c107 (K10467), ab37+c108 (K10468), ab37+c109 (K10469), ab37+c110 (K10470), ab37+c111 (K10471), ab37+c112 (K10472), ab37+c113 (K10473), ab37+c114 (K10474), ab37+c115 (K10475), ab37+c116 (K10476), ab37+c117 (K10477), ab37+c118 (K10478), ab37+c119 (K10479), ab37+c120 (K10480), ab37+c121 (K10481), ab37+c122 (K10482), ab37+c123 (K10483), ab37+c124 (K10484), ab37+c125 (K10485), ab37+c126 (K10486), ab37+c127 (K10487), ab37+c128 (K10488), ab37+c129 (K10489), ab37+c130 (K10490), ab37+c131 (K10491), ab37+c132 (K10492), ab37+c133 (K10493), ab37+c134 (K10494), ab37+c135 (K10495), ab37+c136 (K10496), ab37+c137 (K10497), ab37+c138 (K10498), ab37+c139 (K10499), ab37+c140 (K10500), ab37+c141 (K10501), ab37+c142 (K10502), ab37+c143 (K10503), ab37+c144 (K10504), ab37+c145 (K10505), ab37+c146 (K10506), ab37+c147 (K10507), ab37+c148 (K10508), ab37+c149 (K10509), ab37+c150 (K10510), ab37+c151 (K10511), ab37+c152 (K10512), ab37+c153 (K10513), ab37+c154 (K10514), ab37+c155 (K10515), ab37+c156 (K10516), ab37+c157 (K10517), ab37+c158 (K10518), ab37+c159 (K10519), ab37+c160 (K10520), ab37+c161 (K10521), ab37+c162 (K10522), ab37+c163 (K10523), ab37+c164 (K10524), ab37+c165 (K10525), ab37+c166 (K10526), ab37+c167 (K10527), ab37+c168 (K10528), ab37+c169 (K10529), ab37+c170 (K10530), ab37+c171 (K10531), ab37+c172 (K10532), ab37+c173 (K10533), ab37+c174 (K10534), ab37+c175 (K10535), ab37+c176 (K10536), ab37+c177 (K10537), ab37+c178 (K10538), ab37+c179 (K10539), ab37+c180 (K10540), ab37+c181 (K10541), ab37+c182 (K10542), ab37+c183 (K10543), ab37+c184 (K10544), ab37+c185 (K10545), ab37+c186 (K10546), ab37+c187 (K10547), ab37+c188 (K10548), ab37+c189 (K10549), ab37+c190 (K10550), ab37+c191 (K10551), ab37+c192 (K10552), ab37+c193 (K10553), ab37+c194 (K10554), ab37+c195 (K10555), ab37+c196 (K10556), ab37+c197 (K10557), ab37+c198 (K10558), ab37+c199 (K10559), ab37+c200 (K10560), ab38+c1 (K10561), ab38+c2 (K10562), ab38+c3 (K10563), ab38+c4 (K10564), ab38+c5 (K10565), ab38+c6 (K10566), ab38+c7 (K10567), ab38+c8 (K10568), ab38+c9 (K10569), ab38+c10 (K10570), ab38+c11 (K10571), ab38+c12 (K10572), ab38+c13 (K10573), ab38+c14 (K10574), ab38+c15 (K10575), ab38+c16 (K10576), ab38+c17 (K10577), ab38+c18 (K10578), ab38+c19 (K10579), ab38+c20 (K10580), ab38+c21 (K10581), ab38+c22 (K10582), ab38+c23 (K10583), ab38+c24 (K10584), ab38+c25 (K10585), ab38+c26 (K10586), ab38+c27 (K10587), ab38+c28 (K10588), ab38+c29 (K10589), ab38+c30 (K10590), ab38+c31 (K10591), ab38+c32 (K10592), ab38+c33 (K10593), ab38+c34 (K10594), ab38+c35 (K10595), ab38+c36 (K10596), ab38+c37 (K10597), ab38+c38 (K10598), ab38+c39 (K10599), ab38+c40 (K10600), ab38+c41 (K10601), ab38+c42 (K10602), ab38+c43 (K10603), ab38+c44 (K10604), ab38+c45 (K10605), ab38+c46 (K10606), ab38+c47 (K10607), ab38+c48 (K10608), ab38+c49 (K10609), ab38+c50 (K10610), ab38+c51 (K10611), ab38+c52 (K10612), ab38+c53 (K10613), ab38+c54 (K10614), ab38+c55 (K10615), ab38+c56 (K10616), ab38+c57 (K10617), ab38+c58 (K10618), ab38+c59 (K10619), ab38+c60 (K10620), ab38+c61 (K10621), ab38+c62 (K10622), ab38+c63 (K10623), ab38+c64 (K10624), ab38+c65 (K10625), ab38+c66 (K10626), ab38+c67 (K10627), ab38+c68 (K10628), ab38+c69 (K10629), ab38+c70 (K10630), ab38+c71 (K10631), ab38+c72 (K10632), ab38+c73 (K10633), ab38+c74 (K10634), ab38+c75 (K10635), ab38+c76 (K10636), ab38+c77 (K10637), ab38+c78 (K10638), ab38+c79 (K10639), ab38+c80 (K10640), ab38+c81 (K10641), ab38+c82 (K10642), ab38+c83 (K10643), ab38+c84 (K10644), ab38+c85 (K10645), ab38+c86 (K10646), ab38+c87 (K10647), ab38+c88 (K10648), ab38+c89 (K10649), ab38+c90 (K10650), ab38+c91 (K10651), ab38+c92 (K10652), ab38+c93 (K10653), ab38+c94 (K10654), ab38+c95 (K10655), ab38+c96 (K10656), ab38+c97 (K10657), ab38+c98 (K10658), ab38+c99 (K10659), ab38+c100 (K10660), ab38+c101 (K10661), ab38+c102 (K10662), ab38+c103 (K10663), ab38+c104 (K10664), ab38+c105 (K10665), ab38+c106 (K10666), ab38+c107 (K10667), ab38+c108 (K10668), ab38+c109 (K10669), ab38+c110 (K10670), ab38+c111 (K10671), ab38+c112 (K10672), ab38+c113 (K10673), ab38+c114 (K10674), ab38+c115 (K10675), ab38+c116 (K10676), ab38+c117 (K10677), ab38+c118 (K10678), ab38+c119 (K10679), ab38+c120 (K10680), ab38+c121 (K10681), ab38+c122 (K10682), ab38+c123 (K10683), ab38+c124 (K10684), ab38+c125 (K10685), ab38+c126 (K10686), ab38+c127 (K10687), ab38+c128 (K10688), ab38+c129 (K10689), ab38+c130 (K10690), ab38+c131 (K10691), ab38+c132 (K10692), ab38+c133 (K10693), ab38+c134 (K10694), ab38+c135 (K10695), ab38+c136 (K10696), ab38+c137 (K10697), ab38+c138 (K10698), ab38+c139 (K10699), ab38+c140 (K10700), ab38+c141 (K10701), ab38+c142 (K10702), ab38+c143 (K10703), ab38+c144 (K10704), ab38+c145 (K10705), ab38+c146 (K10706), ab38+c147 (K10707), ab38+c148 (K10708), ab38+c149 (K10709), ab38+c150 (K10710), ab38+c151 (K10711), ab38+c152 (K10712), ab38+c153 (K10713), ab38+c154 (K10714), ab38+c155 (K10715), ab38+c156 (K10716), ab38+c157 (K10717), ab38+c158 (K10718), ab38+c159 (K10719), ab38+c160 (K10720), ab38+c161 (K10721), ab38+c162 (K10722), ab38+c163 (K10723), ab38+c164 (K10724), ab38+c165 (K10725), ab38+c166 (K10726), ab38+c167 (K10727), ab38+c168 (K10728), ab38+c169 (K10729), ab38+c170 (K10730), ab38+c171 (K10731), ab38+c172 (K10732), ab38+c173 (K10733), ab38+c174 (K10734), ab38+c175 (K10735), ab38+c176 (K10736), ab38+c177 (K10737), ab38+c178 (K10738), ab38+c179 (K10739), ab38+c180 (K10740), ab38+c181 (K10741), ab38+c182 (K10742), ab38+c183 (K10743), ab38+c184 (K10744), ab38+c185 (K10745), ab38+c186 (K10746), ab38+c187 (K10747), ab38+c188 (K10748), ab38+c189 (K10749), ab38+c190 (K10750), ab38+c191 (K10751), ab38+c192 (K10752), ab38+c193 (K10753), ab38+c194 (K10754), ab38+c195 (K10755), ab38+c196 (K10756), ab38+c197 (K10757), ab38+c198 (K10758), ab38+c199 (K10759), ab38+c200 (K10760), ab39+c1 (K10761), ab39+c2 (K10762), ab39+c3 (K10763), ab39+c4 (K10764), ab39+c5 (K10765), ab39+c6 (K10766), ab39+c7 (K10767), ab39+c8 (K10768), ab39+c9 (K10769), ab39+c10 (K10770), ab39+c11 (K10771), ab39+c12 (K10772), ab39+c13 (K10773), ab39+c14 (K10774), ab39+c15 (K10775), ab39+c16 (K10776), ab39+c17 (K10777), ab39+c18 (K10778), ab39+c19 (K10779), ab39+c20 (K10780), ab39+c21 (K10781), ab39+c22 (K10782), ab39+c23 (K10783), ab39+c24 (K10784), ab39+c25 (K10785), ab39+c26 (K10786), ab39+c27 (K10787), ab39+c28 (K10788), ab39+c29 (K10789), ab39+c30 (K10790), ab39+c31 (K10791), ab39+c32 (K10792), ab39+c33 (K10793), ab39+c34 (K10794), ab39+c35 (K10795), ab39+c36 (K10796), ab39+c37 (K10797), ab39+c38 (K10798), ab39+c39 (K10799), ab39+c40 (K10800), ab39+c41 (K10801), ab39+c42 (K10802), ab39+c43 (K10803), ab39+c44 (K10804), ab39+c45 (K10805), ab39+c46 (K10806), ab39+c47 (K10807), ab39+c48 (K10808), ab39+c49 (K10809), ab39+c50 (K10810), ab39+c51 (K10811), ab39+c52 (K10812), ab39+c53 (K10813), ab39+c54 (K10814), ab39+c55 (K10815), ab39+c56 (K10816), ab39+c57 (K10817), ab39+c58 (K10818), ab39+c59 (K10819), ab39+c60 (K10820), ab39+c61 (K10821), ab39+c62 (K10822), ab39+c63 (K10823), ab39+c64 (K10824), ab39+c65 (K10825), ab39+c66 (K10826), ab39+c67 (K10827), ab39+c68 (K10828), ab39+c69 (K10829), ab39+c70 (K10830), ab39+c71 (K10831), ab39+c72 (K10832), ab39+c73 (K10833), ab39+c74 (K10834), ab39+c75 (K10835), ab39+c76 (K10836), ab39+c77 (K10837), ab39+c78 (K10838), ab39+c79 (K10839), ab39+c80 (K10840), ab39+c81 (K10841), ab39+c82 (K10842), ab39+c83 (K10843), ab39+c84 (K10844), ab39+c85 (K10845), ab39+c86 (K10846), ab39+c87 (K10847), ab39+c88 (K10848), ab39+c89 (K10849), ab39+c90 (K10850), ab39+c91 (K10851), ab39+c92 (K10852), ab39+c93 (K10853), ab39+c94 (K10854), ab39+c95 (K10855), ab39+c96 (K10856), ab39+c97 (K10857), ab39+c98 (K10858), ab39+c99 (K10859), ab39+c100 (K10860), ab39+c101 (K10861), ab39+c102 (K10862), ab39+c103 (K10863), ab39+c104 (K10864), ab39+c105 (K10865), ab39+c106 (K10866), ab39+c107 (K10867), ab39+c108 (K10868), ab39+c109 (K10869), ab39+c110 (K10870), ab39+c111 (K10871), ab39+c112 (K10872), ab39+c113 (K10873), ab39+c114 (K10874), ab39+c115 (K10875), ab39+c116 (K10876), ab39+c117 (K10877), ab39+c118 (K10878), ab39+c119 (K10879), ab39+c120 (K10880), ab39+c121 (K10881), ab39+c122 (K10882), ab39+c123 (K10883), ab39+c124 (K10884), ab39+c125 (K10885), ab39+c126 (K10886), ab39+c127 (K10887), ab39+c128 (K10888), ab39+c129 (K10889), ab39+c130 (K10890), ab39+c131 (K10891), ab39+c132 (K10892), ab39+c133 (K10893), ab39+c134 (K10894), ab39+c135 (K10895), ab39+c136 (K10896), ab39+c137 (K10897), ab39+c138 (K10898), ab39+c139 (K10899), ab39+c140 (K10900), ab39+c141 (K10901), ab39+c142 (K10902), ab39+c143 (K10903), ab39+c144 (K10904), ab39+c145 (K10905), ab39+c146 (K10906), ab39+c147 (K10907), ab39+c148 (K10908), ab39+c149 (K10909), ab39+c150 (K10910), ab39+c151 (K10911), ab39+c152 (K10912), ab39+c153 (K10913), ab39+c154 (K10914), ab39+c155 (K10915), ab39+c156 (K10916), ab39+c157 (K10917), ab39+c158 (K10918), ab39+c159 (K10919), ab39+c160 (K10920), ab39+c161 (K10921), ab39+c162 (K10922), ab39+c163 (K10923), ab39+c164 (K10924), ab39+c165 (K10925), ab39+c166 (K10926), ab39+c167 (K10927), ab39+c168 (K10928), ab39+c169 (K10929), ab39+c170 (K10930), ab39+c171 (K10931), ab39+c172 (K10932), ab39+c173 (K10933), ab39+c174 (K10934), ab39+c175 (K10935), ab39+c176 (K10936), ab39+c177 (K10937), ab39+c178 (K10938), ab39+c179 (K10939), ab39+c180 (K10940), ab39+c181 (K10941), ab39+c182 (K10942), ab39+c183 (K10943), ab39+c184 (K10944), ab39+c185 (K10945), ab39+c186 (K10946), ab39+c187 (K10947), ab39+c188 (K10948), ab39+c189 (K10949), ab39+c190 (K10950), ab39+c191 (K10951), ab39+c192 (K10952), ab39+c193 (K10953), ab39+c194 (K10954), ab39+c195 (K10955), ab39+c196 (K10956), ab39+c197 (K10957), ab39+c198 (K10958), ab39+c199 (K10959), ab39+c200 (K10960), ab40+c1 (K10961), ab40+c2 (K10962), ab40+c3 (K10963), ab40+c4 (K10964), ab40+c5 (K10965), ab40+c6 (K10966), ab40+c7 (K10967), ab40+c8 (K10968), ab40+c9 (K10969), ab40+c10 (K10970), ab40+c11 (K10971), ab40+c12 (K10972), ab40+c13 (K10973), ab40+c14 (K10974), ab40+c15 (K10975), ab40+c16 (K10976), ab40+c17 (K10977), ab40+c18 (K10978), ab40+c19 (K10979), ab40+c20 (K10980), ab40+c21 (K10981), ab40+c22 (K10982), ab40+c23 (K10983), ab40+c24 (K10984), ab40+c25 (K10985), ab40+c26 (K10986), ab40+c27 (K10987), ab40+c28 (K10988), ab40+c29 (K10989), ab40+c30 (K10990), ab40+c31 (K10991), ab40+c32 (K10992), ab40+c33 (K10993), ab40+c34 (K10994), ab40+c35 (K10995), ab40+c36 (K10996), ab40+c37 (K10997), ab40+c38 (K10998), ab40+c39 (K10999), ab40+c40 (K11000), ab40+c41 (K11001), ab40+c42 (K11002), ab40+c43 (K11003), ab40+c44 (K11004), ab40+c45 (K11005), ab40+c46 (K11006), ab40+c47 (K11007), ab40+c48 (K11008), ab40+c49 (K11009), ab40+c50 (K11010), ab40+c51 (K11011), ab40+c52 (K11012), ab40+c53 (K11013), ab40+c54 (K11014), ab40+c55 (K11015), ab40+c56 (K11016), ab40+c57 (K11017), ab40+c58 (K11018), ab40+c59 (K11019), ab40+c60 (K11020), ab40+c61 (K11021), ab40+c62 (K11022), ab40+c63 (K11023), ab40+c64 (K11024), ab40+c65 (K11025), ab40+c66 (K11026), ab40+c67 (K11027), ab40+c68 (K11028), ab40+c69 (K11029), ab40+c70 (K11030), ab40+c71 (K11031), ab40+c72 (K11032), ab40+c73 (K11033), ab40+c74 (K11034), ab40+c75 (K11035), ab40+c76 (K11036), ab40+c77 (K11037), ab40+c78 (K11038), ab40+c79 (K11039), ab40+c80 (K11040), ab40+c81 (K11041), ab40+c82 (K11042), ab40+c83 (K11043), ab40+c84 (K11044), ab40+c85 (K11045), ab40+c86 (K11046), ab40+c87 (K11047), ab40+c88 (K11048), ab40+c89 (K11049), ab40+c90 (K11050), ab40+c91 (K11051), ab40+c92 (K11052), ab40+c93 (K11053), ab40+c94 (K11054), ab40+c95 (K11055), ab40+c96 (K11056), ab40+c97 (K11057), ab40+c98 (K11058), ab40+c99 (K11059), ab40+c100 (K11060), ab40+c101 (K11061), ab40+c102 (K11062), ab40+c103 (K11063), ab40+c104 (K11064), ab40+c105 (K11065), ab40+c106 (K11066), ab40+c107 (K11067), ab40+c108 (K11068), ab40+c109 (K11069), ab40+c110 (K11070), ab40+c111 (K11071), ab40+c112 (K11072), ab40+c113 (K11073), ab40+c114 (K11074), ab40+c115 (K11075), ab40+c116 (K11076), ab40+c117 (K11077), ab40+c118 (K11078), ab40+c119 (K11079), ab40+c120 (K11080), ab40+c121 (K11081), ab40+c122 (K11082), ab40+c123 (K11083), ab40+c124 (K11084), ab40+c125 (K11085), ab40+c126 (K11086), ab40+c127 (K11087), ab40+c128 (K11088), ab40+c129 (K11089), ab40+c130 (K11090), ab40+c131 (K11091), ab40+c132 (K11092), ab40+c133 (K11093), ab40+c134 (K11094), ab40+c135 (K11095), ab40+c136 (K11096), ab40+c137 (K11097), ab40+c138 (K11098), ab40+c139 (K11099), ab40+c140 (K11100), ab40+c141 (K11101), ab40+c142 (K11102), ab40+c143 (K11103), ab40+c144 (K11104), ab40+c145 (K11105), ab40+c146 (K11106), ab40+c147 (K11107), ab40+c148 (K11108), ab40+c149 (K11109), ab40+c150 (K11110), ab40+c151 (K11111), ab40+c152 (K11112), ab40+c153 (K11113), ab40+c154 (K11114), ab40+c155 (K11115), ab40+c156 (K11116), ab40+c157 (K11117), ab40+c158 (K11118), ab40+c159 (K11119), ab40+c160 (K11120), ab40+c161 (K11121), ab40+c162 (K11122), ab40+c163 (K11123), ab40+c164 (K11124), ab40+c165 (K11125), ab40+c166 (K11126), ab40+c167 (K11127), ab40+c168 (K11128), ab40+c169 (K11129), ab40+c170 (K11130), ab40+c171 (K11131), ab40+c172 (K11132), ab40+c173 (K11133), ab40+c174 (K11134), ab40+c175 (K11135), ab40+c176 (K11136), ab40+c177 (K11137), ab40+c178 (K11138), ab40+c179 (K11139), ab40+c180 (K11140), ab40+c181 (K11141), ab40+c182 (K11142), ab40+c183 (K11143), ab40+c184 (K11144), ab40+c185 (K11145), ab40+c186 (K11146), ab40+c187 (K11147), ab40+c188 (K11148), ab40+c189 (K11149), ab40+c190 (K11150), ab40+c191 (K11151), ab40+c192 (K11152), ab40+c193 (K11153), ab40+c194 (K11154), ab40+c195 (K11155), ab40+c196 (K11156), ab40+c197 (K11157), ab40+c198 (K11158), ab40+c199 (K11159), ab40+c200 (K11160), ab41+c1 (K11161), ab41+c2 (K11162), ab41+c3 (K11163), ab41+c4 (K11164), ab41+c5 (K11165), ab41+c6 (K11166), ab41+c7 (K11167), ab41+c8 (K11168), ab41+c9 (K11169), ab41+c10 (K11170), ab41+c11 (K11171), ab41+c12 (K11172), ab41+c13 (K11173), ab41+c14 (K11174), ab41+c15 (K11175), ab41+c16 (K11176), ab41+c17 (K11177), ab41+c18 (K11178), ab41+c19 (K11179), ab41+c20 (K11180), ab41+c21 (K11181), ab41+c22 (K11182), ab41+c23 (K11183), ab41+c24 (K11184), ab41+c25 (K11185), ab41+c26 (K11186), ab41+c27 (K11187), ab41+c28 (K11188), ab41+c29 (K11189), ab41+c30 (K11190), ab41+c31 (K11191), ab41+c32 (K11192), ab41+c33 (K11193), ab41+c34 (K11194), ab41+c35 (K11195), ab41+c36 (K11196), ab41+c37 (K11197), ab41+c38 (K11198), ab41+c39 (K11199), ab41+c40 (K11200), ab41+c41 (K11201), ab41+c42 (K11202), ab41+c43 (K11203), ab41+c44 (K11204), ab41+c45 (K11205), ab41+c46 (K11206), ab41+c47 (K11207), ab41+c48 (K11208), ab41+c49 (K11209), ab41+c50 (K11210), ab41+c51 (K11211), ab41+c52 (K11212), ab41+c53 (K11213), ab41+c54 (K11214), ab41+c55 (K11215), ab41+c56 (K11216), ab41+c57 (K11217), ab41+c58 (K11218), ab41+c59 (K11219), ab41+c60 (K11220), ab41+c61 (K11221), ab41+c62 (K11222), ab41+c63 (K11223), ab41+c64 (K11224), ab41+c65 (K11225), ab41+c66 (K11226), ab41+c67 (K11227), ab41+c68 (K11228), ab41+c69 (K11229), ab41+c70 (K11230), ab41+c71 (K11231), ab41+c72 (K11232), ab41+c73 (K11233), ab41+c74 (K11234), ab41+c75 (K11235), ab41+c76 (K11236), ab41+c77 (K11237), ab41+c78 (K11238), ab41+c79 (K11239), ab41+c80 (K11240), ab41+c81 (K11241), ab41+c82 (K11242), ab41+c83 (K11243), ab41+c84 (K11244), ab41+c85 (K11245), ab41+c86 (K11246), ab41+c87 (K11247), ab41+c88 (K11248), ab41+c89 (K11249), ab41+c90 (K11250), ab41+c91 (K11251), ab41+c92 (K11252), ab41+c93 (K11253), ab41+c94 (K11254), ab41+c95 (K11255), ab41+c96 (K11256), ab41+c97 (K11257), ab41+c98 (K11258), ab41+c99 (K11259), ab41+c100 (K11260), ab41+c101 (K11261), ab41+c102 (K11262), ab41+c103 (K11263), ab41+c104 (K11264), ab41+c105 (K11265), ab41+c106 (K11266), ab41+c107 (K11267), ab41+c108 (K11268), ab41+c109 (K11269), ab41+c110 (K11270), ab41+c111 (K11271), ab41+c112 (K11272), ab41+c113 (K11273), ab41+c114 (K11274), ab41+c115 (K11275), ab41+c116 (K11276), ab41+c117 (K11277), ab41+c118 (K11278), ab41+c119 (K11279), ab41+c120 (K11280), ab41+c121 (K11281), ab41+c122 (K11282), ab41+c123 (K11283), ab41+c124 (K11284), ab41+c125 (K11285), ab41+c126 (K11286), ab41+c127 (K11287), ab41+c128 (K11288), ab41+c129 (K11289), ab41+c130 (K11290), ab41+c131 (K11291), ab41+c132 (K11292), ab41+c133 (K11293), ab41+c134 (K11294), ab41+c135 (K11295), ab41+c136 (K11296), ab41+c137 (K11297), ab41+c138 (K11298), ab41+c139 (K11299), ab41+c140 (K11300), ab41+c141 (K11301), ab41+c142 (K11302), ab41+c143 (K11303), ab41+c144 (K11304), ab41+c145 (K11305), ab41+c146 (K11306), ab41+c147 (K11307), ab41+c148 (K11308), ab41+c149 (K11309), ab41+c150 (K11310), ab41+c151 (K11311), ab41+c152 (K11312), ab41+c153 (K11313), ab41+c154 (K11314), ab41+c155 (K11315), ab41+c156 (K11316), ab41+c157 (K11317), ab41+c158 (K11318), ab41+c159 (K11319), ab41+c160 (K11320), ab41+c161 (K11321), ab41+c162 (K11322), ab41+c163 (K11323), ab41+c164 (K11324), ab41+c165 (K11325), ab41+c166 (K11326), ab41+c167 (K11327), ab41+c168 (K11328), ab41+c169 (K11329), ab41+c170 (K11330), ab41+c171 (K11331), ab41+c172 (K11332), ab41+c173 (K11333), ab41+c174 (K11334), ab41+c175 (K11335), ab41+c176 (K11336), ab41+c177 (K11337), ab41+c178 (K11338), ab41+c179 (K11339), ab41+c180 (K11340), ab41+c181 (K11341), ab41+c182 (K11342), ab41+c183 (K11343), ab41+c184 (K11344), ab41+c185 (K11345), ab41+c186 (K11346), ab41+c187 (K11347), ab41+c188 (K11348), ab41+c189 (K11349), ab41+c190 (K11350), ab41+c191 (K11351), ab41+c192 (K11352), ab41+c193 (K11353), ab41+c194 (K11354), ab41+c195 (K11355), ab41+c196 (K11356), ab41+c197 (K11357), ab41+c198 (K11358), ab41+c199 (K11359), ab41+c200 (K11360), ab42+c1 (K11361), ab42+c2 (K11362), ab42+c3 (K11363), ab42+c4 (K11364), ab42+c5 (K11365), ab42+c6 (K11366), ab42+c7 (K11367), ab42+c8 (K11368), ab42+c9 (K11369), ab42+c10 (K11370), ab42+c11 (K11371), ab42+c12 (K11372), ab42+c13 (K11373), ab42+c14 (K11374), ab42+c15 (K11375), ab42+c16 (K11376), ab42+c17 (K11377), ab42+c18 (K11378), ab42+c19 (K11379), ab42+c20 (K11380), ab42+c21 (K11381), ab42+c22 (K11382), ab42+c23 (K11383), ab42+c24 (K11384), ab42+c25 (K11385), ab42+c26 (K11386), ab42+c27 (K11387), ab42+c28 (K11388), ab42+c29 (K11389), ab42+c30 (K11390), ab42+c31 (K11391), ab42+c32 (K11392), ab42+c33 (K11393), ab42+c34 (K11394), ab42+c35 (K11395), ab42+c36 (K11396), ab42+c37 (K11397), ab42+c38 (K11398), ab42+c39 (K11399), ab42+c40 (K11400), ab42+c41 (K11401), ab42+c42 (K11402), ab42+c43 (K11403), ab42+c44 (K11404), ab42+c45 (K11405), ab42+c46 (K11406), ab42+c47 (K11407), ab42+c48 (K11408), ab42+c49 (K11409), ab42+c50 (K11410), ab42+c51 (K11411), ab42+c52 (K11412), ab42+c53 (K11413), ab42+c54 (K11414), ab42+c55 (K11415), ab42+c56 (K11416), ab42+c57 (K11417), ab42+c58 (K11418), ab42+c59 (K11419), ab42+c60 (K11420), ab42+c61 (K11421), ab42+c62 (K11422), ab42+c63 (K11423), ab42+c64 (K11424), ab42+c65 (K11425), ab42+c66 (K11426), ab42+c67 (K11427), ab42+c68 (K11428), ab42+c69 (K11429), ab42+c70 (K11430), ab42+c71 (K11431), ab42+c72 (K11432), ab42+c73 (K11433), ab42+c74 (K11434), ab42+c75 (K11435), ab42+c76 (K11436), ab42+c77 (K11437), ab42+c78 (K11438), ab42+c79 (K11439), ab42+c80 (K11440), ab42+c81 (K11441), ab42+c82 (K11442), ab42+c83 (K11443), ab42+c84 (K11444), ab42+c85 (K11445), ab42+c86 (K11446), ab42+c87 (K11447), ab42+c88 (K11448), ab42+c89 (K11449), ab42+c90 (K11450), ab42+c91 (K11451), ab42+c92 (K11452), ab42+c93 (K11453), ab42+c94 (K11454), ab42+c95 (K11455), ab42+c96 (K11456), ab42+c97 (K11457), ab42+c98 (K11458), ab42+c99 (K11459), ab42+c100 (K11460), ab42+c101 (K11461), ab42+c102 (K11462), ab42+c103 (K11463), ab42+c104 (K11464), ab42+c105 (K11465), ab42+c106 (K11466), ab42+c107 (K11467), ab42+c108 (K11468), ab42+c109 (K11469), ab42+c110 (K11470), ab42+c111 (K11471), ab42+c112 (K11472), ab42+c113 (K11473), ab42+c114 (K11474), ab42+c115 (K11475), ab42+c116 (K11476), ab42+c117 (K11477), ab42+c118 (K11478), ab42+c119 (K11479), ab42+c120 (K11480), ab42+c121 (K11481), ab42+c122 (K11482), ab42+c123 (K11483), ab42+c124 (K11484), ab42+c125 (K11485), ab42+c126 (K11486), ab42+c127 (K11487), ab42+c128 (K11488), ab42+c129 (K11489), ab42+c130 (K11490), ab42+c131 (K11491), ab42+c132 (K11492), ab42+c133 (K11493), ab42+c134 (K11494), ab42+c135 (K11495), ab42+c136 (K11496), ab42+c137 (K11497), ab42+c138 (K11498), ab42+c139 (K11499), ab42+c140 (K11500), ab42+c141 (K11501), ab42+c142 (K11502), ab42+c143 (K11503), ab42+c144 (K11504), ab42+c145 (K11505), ab42+c146 (K11506), ab42+c147 (K11507), ab42+c148 (K11508), ab42+c149 (K11509), ab42+c150 (K11510), ab42+c151 (K11511), ab42+c152 (K11512), ab42+c153 (K11513), ab42+c154 (K11514), ab42+c155 (K11515), ab42+c156 (K11516), ab42+c157 (K11517), ab42+c158 (K11518), ab42+c159 (K11519), ab42+c160 (K11520), ab42+c161 (K11521), ab42+c162 (K11522), ab42+c163 (K11523), ab42+c164 (K11524), ab42+c165 (K11525), ab42+c166 (K11526), ab42+c167 (K11527), ab42+c168 (K11528), ab42+c169 (K11529), ab42+c170 (K11530), ab42+c171 (K11531), ab42+c172 (K11532), ab42+c173 (K11533), ab42+c174 (K11534), ab42+c175 (K11535), ab42+c176 (K11536), ab42+c177 (K11537), ab42+c178 (K11538), ab42+c179 (K11539), ab42+c180 (K11540), ab42+c181 (K11541), ab42+c182 (K11542), ab42+c183 (K11543), ab42+c184 (K11544), ab42+c185 (K11545), ab42+c186 (K11546), ab42+c187 (K11547), ab42+c188 (K11548), ab42+c189 (K11549), ab42+c190 (K11550), ab42+c191 (K11551), ab42+c192 (K11552), ab42+c193 (K11553), ab42+c194 (K11554), ab42+c195 (K11555), ab42+c196 (K11556), ab42+c197 (K11557), ab42+c198 (K11558), ab42+c199 (K11559), ab42+c200 (K11560), ab43+c1 (K11561), ab43+c2 (K11562), ab43+c3 (K11563), ab43+c4 (K11564), ab43+c5 (K11565), ab43+c6 (K11566), ab43+c7 (K11567), ab43+c8 (K11568), ab43+c9 (K11569), ab43+c10 (K11570), ab43+c11 (K11571), ab43+c12 (K11572), ab43+c13 (K11573), ab43+c14 (K11574), ab43+c15 (K11575), ab43+c16 (K11576), ab43+c17 (K11577), ab43+c18 (K11578), ab43+c19 (K11579), ab43+c20 (K11580), ab43+c21 (K11581), ab43+c22 (K11582), ab43+c23 (K11583), ab43+c24 (K11584), ab43+c25 (K11585), ab43+c26 (K11586), ab43+c27 (K11587), ab43+c28 (K11588), ab43+c29 (K11589), ab43+c30 (K11590), ab43+c31 (K11591), ab43+c32 (K11592), ab43+c33 (K11593), ab43+c34 (K11594), ab43+c35 (K11595), ab43+c36 (K11596), ab43+c37 (K11597), ab43+c38 (K11598), ab43+c39 (K11599), ab43+c40 (K11600), ab43+c41 (K11601), ab43+c42 (K11602), ab43+c43 (K11603), ab43+c44 (K11604), ab43+c45 (K11605), ab43+c46 (K11606), ab43+c47 (K11607), ab43+c48 (K11608), ab43+c49 (K11609), ab43+c50 (K11610), ab43+c51 (K11611), ab43+c52 (K11612), ab43+c53 (K11613), ab43+c54 (K11614), ab43+c55 (K11615), ab43+c56 (K11616), ab43+c57 (K11617), ab43+c58 (K11618), ab43+c59 (K11619), ab43+c60 (K11620), ab43+c61 (K11621), ab43+c62 (K11622), ab43+c63 (K11623), ab43+c64 (K11624), ab43+c65 (K11625), ab43+c66 (K11626), ab43+c67 (K11627), ab43+c68 (K11628), ab43+c69 (K11629), ab43+c70 (K11630), ab43+c71 (K11631), ab43+c72 (K11632), ab43+c73 (K11633), ab43+c74 (K11634), ab43+c75 (K11635), ab43+c76 (K11636), ab43+c77 (K11637), ab43+c78 (K11638), ab43+c79 (K11639), ab43+c80 (K11640), ab43+c81 (K11641), ab43+c82 (K11642), ab43+c83 (K11643), ab43+c84 (K11644), ab43+c85 (K11645), ab43+c86 (K11646), ab43+c87 (K11647), ab43+c88 (K11648), ab43+c89 (K11649), ab43+c90 (K11650), ab43+c91 (K11651), ab43+c92 (K11652), ab43+c93 (K11653), ab43+c94 (K11654), ab43+c95 (K11655), ab43+c96 (K11656), ab43+c97 (K11657), ab43+c98 (K11658), ab43+c99 (K11659), ab43+c100 (K11660), ab43+c101 (K11661), ab43+c102 (K11662), ab43+c103 (K11663), ab43+c104 (K11664), ab43+c105 (K11665), ab43+c106 (K11666), ab43+c107 (K11667), ab43+c108 (K11668), ab43+c109 (K11669), ab43+c110 (K11670), ab43+c111 (K11671), ab43+c112 (K11672), ab43+c113 (K11673), ab43+c114 (K11674), ab43+c115 (K11675), ab43+c116 (K11676), ab43+c117 (K11677), ab43+c118 (K11678), ab43+c119 (K11679), ab43+c120 (K11680), ab43+c121 (K11681), ab43+c122 (K11682), ab43+c123 (K11683), ab43+c124 (K11684), ab43+c125 (K11685), ab43+c126 (K11686), ab43+c127 (K11687), ab43+c128 (K11688), ab43+c129 (K11689), ab43+c130 (K11690), ab43+c131 (K11691), ab43+c132 (K11692), ab43+c133 (K11693), ab43+c134 (K11694), ab43+c135 (K11695), ab43+c136 (K11696), ab43+c137 (K11697), ab43+c138 (K11698), ab43+c139 (K11699), ab43+c140 (K11700), ab43+c141 (K11701), ab43+c142 (K11702), ab43+c143 (K11703), ab43+c144 (K11704), ab43+c145 (K11705), ab43+c146 (K11706), ab43+c147 (K11707), ab43+c148 (K11708), ab43+c149 (K11709), ab43+c150 (K11710), ab43+c151 (K11711), ab43+c152 (K11712), ab43+c153 (K11713), ab43+c154 (K11714), ab43+c155 (K11715), ab43+c156 (K11716), ab43+c157 (K11717), ab43+c158 (K11718), ab43+c159 (K11719), ab43+c160 (K11720), ab43+c161 (K11721), ab43+c162 (K11722), ab43+c163 (K11723), ab43+c164 (K11724), ab43+c165 (K11725), ab43+c166 (K11726), ab43+c167 (K11727), ab43+c168 (K11728), ab43+c169 (K11729), ab43+c170 (K11730), ab43+c171 (K11731), ab43+c172 (K11732), ab43+c173 (K11733), ab43+c174 (K11734), ab43+c175 (K11735), ab43+c176 (K11736), ab43+c177 (K11737), ab43+c178 (K11738), ab43+c179 (K11739), ab43+c180 (K11740), ab43+c181 (K11741), ab43+c182 (K11742), ab43+c183 (K11743), ab43+c184 (K11744), ab43+c185 (K11745), ab43+c186 (K11746), ab43+c187 (K11747), ab43+c188 (K11748), ab43+c189 (K11749), ab43+c190 (K11750), ab43+c191 (K11751), ab43+c192 (K11752), ab43+c193 (K11753), ab43+c194 (K11754), ab43+c195 (K11755), ab43+c196 (K11756), ab43+c197 (K11757), ab43+c198 (K11758), ab43+c199 (K11759), ab43+c200 (K11760), ab44+c1 (K11761), ab44+c2 (K11762), ab44+c3 (K11763), ab44+c4 (K11764), ab44+c5 (K11765), ab44+c6 (K11766), ab44+c7 (K11767), ab44+c8 (K11768), ab44+c9 (K11769), ab44+c10 (K11770), ab44+c11 (K11771), ab44+c12 (K11772), ab44+c13 (K11773), ab44+c14 (K11774), ab44+c15 (K11775), ab44+c16 (K11776), ab44+c17 (K11777), ab44+c18 (K11778), ab44+c19 (K11779), ab44+c20 (K11780), ab44+c21 (K11781), ab44+c22 (K11782), ab44+c23 (K11783), ab44+c24 (K11784), ab44+c25 (K11785), ab44+c26 (K11786), ab44+c27 (K11787), ab44+c28 (K11788), ab44+c29 (K11789), ab44+c30 (K11790), ab44+c31 (K11791), ab44+c32 (K11792), ab44+c33 (K11793), ab44+c34 (K11794), ab44+c35 (K11795), ab44+c36 (K11796), ab44+c37 (K11797), ab44+c38 (K11798), ab44+c39 (K11799), ab44+c40 (K11800), ab44+c41 (K11801), ab44+c42 (K11802), ab44+c43 (K11803), ab44+c44 (K11804), ab44+c45 (K11805), ab44+c46 (K11806), ab44+c47 (K11807), ab44+c48 (K11808), ab44+c49 (K11809), ab44+c50 (K11810), ab44+c51 (K11811), ab44+c52 (K11812), ab44+c53 (K11813), ab44+c54 (K11814), ab44+c55 (K11815), ab44+c56 (K11816), ab44+c57 (K11817), ab44+c58 (K11818), ab44+c59 (K11819), ab44+c60 (K11820), ab44+c61 (K11821), ab44+c62 (K11822), ab44+c63 (K11823), ab44+c64 (K11824), ab44+c65 (K11825), ab44+c66 (K11826), ab44+c67 (K11827), ab44+c68 (K11828), ab44+c69 (K11829), ab44+c70 (K11830), ab44+c71 (K11831), ab44+c72 (K11832), ab44+c73 (K11833), ab44+c74 (K11834), ab44+c75 (K11835), ab44+c76 (K11836), ab44+c77 (K11837), ab44+c78 (K11838), ab44+c79 (K11839), ab44+c80 (K11840), ab44+c81 (K11841), ab44+c82 (K11842), ab44+c83 (K11843), ab44+c84 (K11844), ab44+c85 (K11845), ab44+c86 (K11846), ab44+c87 (K11847), ab44+c88 (K11848), ab44+c89 (K11849), ab44+c90 (K11850), ab44+c91 (K11851), ab44+c92 (K11852), ab44+c93 (K11853), ab44+c94 (K11854), ab44+c95 (K11855), ab44+c96 (K11856), ab44+c97 (K11857), ab44+c98 (K11858), ab44+c99 (K11859), ab44+c100 (K11860), ab44+c101 (K11861), ab44+c102 (K11862), ab44+c103 (K11863), ab44+c104 (K11864), ab44+c105 (K11865), ab44+c106 (K11866), ab44+c107 (K11867), ab44+c108 (K11868), ab44+c109 (K11869), ab44+c110 (K11870), ab44+c111 (K11871), ab44+c112 (K11872), ab44+c113 (K11873), ab44+c114 (K11874), ab44+c115 (K11875), ab44+c116 (K11876), ab44+c117 (K11877), ab44+c118 (K11878), ab44+c119 (K11879), ab44+c120 (K11880), ab44+c121 (K11881), ab44+c122 (K11882), ab44+c123 (K11883), ab44+c124 (K11884), ab44+c125 (K11885), ab44+c126 (K11886), ab44+c127 (K11887), ab44+c128 (K11888), ab44+c129 (K11889), ab44+c130 (K11890), ab44+c131 (K11891), ab44+c132 (K11892), ab44+c133 (K11893), ab44+c134 (K11894), ab44+c135 (K11895), ab44+c136 (K11896), ab44+c137 (K11897), ab44+c138 (K11898), ab44+c139 (K11899), ab44+c140 (K11900), ab44+c141 (K11901), ab44+c142 (K11902), ab44+c143 (K11903), ab44+c144 (K11904), ab44+c145 (K11905), ab44+c146 (K11906), ab44+c147 (K11907), ab44+c148 (K11908), ab44+c149 (K11909), ab44+c150 (K11910), ab44+c151 (K11911), ab44+c152 (K11912), ab44+c153 (K11913), ab44+c154 (K11914), ab44+c155 (K11915), ab44+c156 (K11916), ab44+c157 (K11917), ab44+c158 (K11918), ab44+c159 (K11919), ab44+c160 (K11920), ab44+c161 (K11921), ab44+c162 (K11922), ab44+c163 (K11923), ab44+c164 (K11924), ab44+c165 (K11925), ab44+c166 (K11926), ab44+c167 (K11927), ab44+c168 (K11928), ab44+c169 (K11929), ab44+c170 (K11930), ab44+c171 (K11931), ab44+c172 (K11932), ab44+c173 (K11933), ab44+c174 (K11934), ab44+c175 (K11935), ab44+c176 (K11936), ab44+c177 (K11937), ab44+c178 (K11938), ab44+c179 (K11939), ab44+c180 (K11940), ab44+c181 (K11941), ab44+c182 (K11942), ab44+c183 (K11943), ab44+c184 (K11944), ab44+c185 (K11945), ab44+c186 (K11946), ab44+c187 (K11947), ab44+c188 (K11948), ab44+c189 (K11949), ab44+c190 (K11950), ab44+c191 (K11951), ab44+c192 (K11952), ab44+c193 (K11953), ab44+c194 (K11954), ab44+c195 (K11955), ab44+c196 (K11956), ab44+c197 (K11957), ab44+c198 (K11958), ab44+c199 (K11959), ab44+c200 (K11960), ab45+c1 (K11961), ab45+c2 (K11962), ab45+c3 (K11963), ab45+c4 (K11964), ab45+c5 (K11965), ab45+c6 (K11966), ab45+c7 (K11967), ab45+c8 (K11968), ab45+c9 (K11969), ab45+c10 (K11970), ab45+c11 (K11971), ab45+c12 (K11972), ab45+c13 (K11973), ab45+c14 (K11974), ab45+c15 (K11975), ab45+c16 (K11976), ab45+c17 (K11977), ab45+c18 (K11978), ab45+c19 (K11979), ab45+c20 (K11980), ab45+c21 (K11981), ab45+c22 (K11982), ab45+c23 (K11983), ab45+c24 (K11984), ab45+c25 (K11985), ab45+c26 (K11986), ab45+c27 (K11987), ab45+c28 (K11988), ab45+c29 (K11989), ab45+c30 (K11990), ab45+c31 (K11991), ab45+c32 (K11992), ab45+c33 (K11993), ab45+c34 (K11994), ab45+c35 (K11995), ab45+c36 (K11996), ab45+c37 (K11997), ab45+c38 (K11998), ab45+c39 (K11999), ab45+c40 (K12000), ab45+c41 (K12001), ab45+c42 (K12002), ab45+c43 (K12003), ab45+c44 (K12004), ab45+c45 (K12005), ab45+c46 (K12006), ab45+c47 (K12007), ab45+c48 (K12008), ab45+c49 (K12009), ab45+c50 (K12010), ab45+c51 (K12011), ab45+c52 (K12012), ab45+c53 (K12013), ab45+c54 (K12014), ab45+c55 (K12015), ab45+c56 (K12016), ab45+c57 (K12017), ab45+c58 (K12018), ab45+c59 (K12019), ab45+c60 (K12020), ab45+c61 (K12021), ab45+c62 (K12022), ab45+c63 (K12023), ab45+c64 (K12024), ab45+c65 (K12025), ab45+c66 (K12026), ab45+c67 (K12027), ab45+c68 (K12028), ab45+c69 (K12029), ab45+c70 (K12030), ab45+c71 (K12031), ab45+c72 (K12032), ab45+c73 (K12033), ab45+c74 (K12034), ab45+c75 (K12035), ab45+c76 (K12036), ab45+c77 (K12037), ab45+c78 (K12038), ab45+c79 (K12039), ab45+c80 (K12040), ab45+c81 (K12041), ab45+c82 (K12042), ab45+c83 (K12043), ab45+c84 (K12044), ab45+c85 (K12045), ab45+c86 (K12046), ab45+c87 (K12047), ab45+c88 (K12048), ab45+c89 (K12049), ab45+c90 (K12050), ab45+c91 (K12051), ab45+c92 (K12052), ab45+c93 (K12053), ab45+c94 (K12054), ab45+c95 (K12055), ab45+c96 (K12056), ab45+c97 (K12057), ab45+c98 (K12058), ab45+c99 (K12059), ab45+c100 (K12060), ab45+c101 (K12061), ab45+c102 (K12062), ab45+c103 (K12063), ab45+c104 (K12064), ab45+c105 (K12065), ab45+c106 (K12066), ab45+c107 (K12067), ab45+c108 (K12068), ab45+c109 (K12069), ab45+c110 (K12070), ab45+c111 (K12071), ab45+c112 (K12072), ab45+c113 (K12073), ab45+c114 (K12074), ab45+c115 (K12075), ab45+c116 (K12076), ab45+c117 (K12077), ab45+c118 (K12078), ab45+c119 (K12079), ab45+c120 (K12080), ab45+c121 (K12081), ab45+c122 (K12082), ab45+c123 (K12083), ab45+c124 (K12084), ab45+c125 (K12085), ab45+c126 (K12086), ab45+c127 (K12087), ab45+c128 (K12088), ab45+c129 (K12089), ab45+c130 (K12090), ab45+c131 (K12091), ab45+c132 (K12092), ab45+c133 (K12093), ab45+c134 (K12094), ab45+c135 (K12095), ab45+c136 (K12096), ab45+c137 (K12097), ab45+c138 (K12098), ab45+c139 (K12099), ab45+c140 (K12100), ab45+c141 (K12101), ab45+c142 (K12102), ab45+c143 (K12103), ab45+c144 (K12104), ab45+c145 (K12105), ab45+c146 (K12106), ab45+c147 (K12107), ab45+c148 (K12108), ab45+c149 (K12109), ab45+c150 (K12110), ab45+c151 (K12111), ab45+c152 (K12112), ab45+c153 (K12113), ab45+c154 (K12114), ab45+c155 (K12115), ab45+c156 (K12116), ab45+c157 (K12117), ab45+c158 (K12118), ab45+c159 (K12119), ab45+c160 (K12120), ab45+c161 (K12121), ab45+c162 (K12122), ab45+c163 (K12123), ab45+c164 (K12124), ab45+c165 (K12125), ab45+c166 (K12126), ab45+c167 (K12127), ab45+c168 (K12128), ab45+c169 (K12129), ab45+c170 (K12130), ab45+c171 (K12131), ab45+c172 (K12132), ab45+c173 (K12133), ab45+c174 (K12134), ab45+c175 (K12135), ab45+c176 (K12136), ab45+c177 (K12137), ab45+c178 (K12138), ab45+c179 (K12139), ab45+c180 (K12140), ab45+c181 (K12141), ab45+c182 (K12142), ab45+c183 (K12143), ab45+c184 (K12144), ab45+c185 (K12145), ab45+c186 (K12146), ab45+c187 (K12147), ab45+c188 (K12148), ab45+c189 (K12149), ab45+c190 (K12150), ab45+c191 (K12151), ab45+c192 (K12152), ab45+c193 (K12153), ab45+c194 (K12154), ab45+c195 (K12155), ab45+c196 (K12156), ab45+c197 (K12157), ab45+c198 (K12158), ab45+c199 (K12159), ab45+c200 (K12160), ab46+c1 (K12161), ab46+c2 (K12162), ab46+c3 (K12163), ab46+c4 (K12164), ab46+c5 (K12165), ab46+c6 (K12166), ab46+c7 (K12167), ab46+c8 (K12168), ab46+c9 (K12169), ab46+c10 (K12170), ab46+c11 (K12171), ab46+c12 (K12172), ab46+c13 (K12173), ab46+c14 (K12174), ab46+c15 (K12175), ab46+c16 (K12176), ab46+c17 (K12177), ab46+c18 (K12178), ab46+c19 (K12179), ab46+c20 (K12180), ab46+c21 (K12181), ab46+c22 (K12182), ab46+c23 (K12183), ab46+c24 (K12184), ab46+c25 (K12185), ab46+c26 (K12186), ab46+c27 (K12187), ab46+c28 (K12188), ab46+c29 (K12189), ab46+c30 (K12190), ab46+c31 (K12191), ab46+c32 (K12192), ab46+c33 (K12193), ab46+c34 (K12194), ab46+c35 (K12195), ab46+c36 (K12196), ab46+c37 (K12197), ab46+c38 (K12198), ab46+c39 (K12199), ab46+c40 (K12200), ab46+c41 (K12201), ab46+c42 (K12202), ab46+c43 (K12203), ab46+c44 (K12204), ab46+c45 (K12205), ab46+c46 (K12206), ab46+c47 (K12207), ab46+c48 (K12208), ab46+c49 (K12209), ab46+c50 (K12210), ab46+c51 (K12211), ab46+c52 (K12212), ab46+c53 (K12213), ab46+c54 (K12214), ab46+c55 (K12215), ab46+c56 (K12216), ab46+c57 (K12217), ab46+c58 (K12218), ab46+c59 (K12219), ab46+c60 (K12220), ab46+c61 (K12221), ab46+c62 (K12222), ab46+c63 (K12223), ab46+c64 (K12224), ab46+c65 (K12225), ab46+c66 (K12226), ab46+c67 (K12227), ab46+c68 (K12228), ab46+c69 (K12229), ab46+c70 (K12230), ab46+c71 (K12231), ab46+c72 (K12232), ab46+c73 (K12233), ab46+c74 (K12234), ab46+c75 (K12235), ab46+c76 (K12236), ab46+c77 (K12237), ab46+c78 (K12238), ab46+c79 (K12239), ab46+c80 (K12240), ab46+c81 (K12241), ab46+c82 (K12242), ab46+c83 (K12243), ab46+c84 (K12244), ab46+c85 (K12245), ab46+c86 (K12246), ab46+c87 (K12247), ab46+c88 (K12248), ab46+c89 (K12249), ab46+c90 (K12250), ab46+c91 (K12251), ab46+c92 (K12252), ab46+c93 (K12253), ab46+c94 (K12254), ab46+c95 (K12255), ab46+c96 (K12256), ab46+c97 (K12257), ab46+c98 (K12258), ab46+c99 (K12259), ab46+c100 (K12260), ab46+c101 (K12261), ab46+c102 (K12262), ab46+c103 (K12263), ab46+c104 (K12264), ab46+c105 (K12265), ab46+c106 (K12266), ab46+c107 (K12267), ab46+c108 (K12268), ab46+c109 (K12269), ab46+c110 (K12270), ab46+c111 (K12271), ab46+c112 (K12272), ab46+c113 (K12273), ab46+c114 (K12274), ab46+c115 (K12275), ab46+c116 (K12276), ab46+c117 (K12277), ab46+c118 (K12278), ab46+c119 (K12279), ab46+c120 (K12280), ab46+c121 (K12281), ab46+c122 (K12282), ab46+c123 (K12283), ab46+c124 (K12284), ab46+c125 (K12285), ab46+c126 (K12286), ab46+c127 (K12287), ab46+c128 (K12288), ab46+c129 (K12289), ab46+c130 (K12290), ab46+c131 (K12291), ab46+c132 (K12292), ab46+c133 (K12293), ab46+c134 (K12294), ab46+c135 (K12295), ab46+c136 (K12296), ab46+c137 (K12297), ab46+c138 (K12298), ab46+c139 (K12299), ab46+c140 (K12300), ab46+c141 (K12301), ab46+c142 (K12302), ab46+c143 (K12303), ab46+c144 (K12304), ab46+c145 (K12305), ab46+c146 (K12306), ab46+c147 (K12307), ab46+c148 (K12308), ab46+c149 (K12309), ab46+c150 (K12310), ab46+c151 (K12311), ab46+c152 (K12312), ab46+c153 (K12313), ab46+c154 (K12314), ab46+c155 (K12315), ab46+c156 (K12316), ab46+c157 (K12317), ab46+c158 (K12318), ab46+c159 (K12319), ab46+c160 (K12320), ab46+c161 (K12321), ab46+c162 (K12322), ab46+c163 (K12323), ab46+c164 (K12324), ab46+c165 (K12325), ab46+c166 (K12326), ab46+c167 (K12327), ab46+c168 (K12328), ab46+c169 (K12329), ab46+c170 (K12330), ab46+c171 (K12331), ab46+c172 (K12332), ab46+c173 (K12333), ab46+c174 (K12334), ab46+c175 (K12335), ab46+c176 (K12336), ab46+c177 (K12337), ab46+c178 (K12338), ab46+c179 (K12339), ab46+c180 (K12340), ab46+c181 (K12341), ab46+c182 (K12342), ab46+c183 (K12343), ab46+c184 (K12344), ab46+c185 (K12345), ab46+c186 (K12346), ab46+c187 (K12347), ab46+c188 (K12348), ab46+c189 (K12349), ab46+c190 (K12350), ab46+c191 (K12351), ab46+c192 (K12352), ab46+c193 (K12353), ab46+c194 (K12354), ab46+c195 (K12355), ab46+c196 (K12356), ab46+c197 (K12357), ab46+c198 (K12358), ab46+c199 (K12359), ab46+c200 (K12360), ab47+c1 (K12361), ab47+c2 (K12362), ab47+c3 (K12363), ab47+c4 (K12364), ab47+c5 (K12365), ab47+c6 (K12366), ab47+c7 (K12367), ab47+c8 (K12368), ab47+c9 (K12369), ab47+c10 (K12370), ab47+c11 (K12371), ab47+c12 (K12372), ab47+c13 (K12373), ab47+c14 (K12374), ab47+c15 (K12375), ab47+c16 (K12376), ab47+c17 (K12377), ab47+c18 (K12378), ab47+c19 (K12379), ab47+c20 (K12380), ab47+c21 (K12381), ab47+c22 (K12382), ab47+c23 (K12383), ab47+c24 (K12384), ab47+c25 (K12385), ab47+c26 (K12386), ab47+c27 (K12387), ab47+c28 (K12388), ab47+c29 (K12389), ab47+c30 (K12390), ab47+c31 (K12391), ab47+c32 (K12392), ab47+c33 (K12393), ab47+c34 (K12394), ab47+c35 (K12395), ab47+c36 (K12396), ab47+c37 (K12397), ab47+c38 (K12398), ab47+c39 (K12399), ab47+c40 (K12400), ab47+c41 (K12401), ab47+c42 (K12402), ab47+c43 (K12403), ab47+c44 (K12404), ab47+c45 (K12405), ab47+c46 (K12406), ab47+c47 (K12407), ab47+c48 (K12408), ab47+c49 (K12409), ab47+c50 (K12410), ab47+c51 (K12411), ab47+c52 (K12412), ab47+c53 (K12413), ab47+c54 (K12414), ab47+c55 (K12415), ab47+c56 (K12416), ab47+c57 (K12417), ab47+c58 (K12418), ab47+c59 (K12419), ab47+c60 (K12420), ab47+c61 (K12421), ab47+c62 (K12422), ab47+c63 (K12423), ab47+c64 (K12424), ab47+c65 (K12425), ab47+c66 (K12426), ab47+c67 (K12427), ab47+c68 (K12428), ab47+c69 (K12429), ab47+c70 (K12430), ab47+c71 (K12431), ab47+c72 (K12432), ab47+c73 (K12433), ab47+c74 (K12434), ab47+c75 (K12435), ab47+c76 (K12436), ab47+c77 (K12437), ab47+c78 (K12438), ab47+c79 (K12439), ab47+c80 (K12440), ab47+c81 (K12441), ab47+c82 (K12442), ab47+c83 (K12443), ab47+c84 (K12444), ab47+c85 (K12445), ab47+c86 (K12446), ab47+c87 (K12447), ab47+c88 (K12448), ab47+c89 (K12449), ab47+c90 (K12450), ab47+c91 (K12451), ab47+c92 (K12452), ab47+c93 (K12453), ab47+c94 (K12454), ab47+c95 (K12455), ab47+c96 (K12456), ab47+c97 (K12457), ab47+c98 (K12458), ab47+c99 (K12459), ab47+c100 (K12460), ab47+c101 (K12461), ab47+c102 (K12462), ab47+c103 (K12463), ab47+c104 (K12464), ab47+c105 (K12465), ab47+c106 (K12466), ab47+c107 (K12467), ab47+c108 (K12468), ab47+c109 (K12469), ab47+c110 (K12470), ab47+c111 (K12471), ab47+c112 (K12472), ab47+c113 (K12473), ab47+c114 (K12474), ab47+c115 (K12475), ab47+c116 (K12476), ab47+c117 (K12477), ab47+c118 (K12478), ab47+c119 (K12479), ab47+c120 (K12480), ab47+c121 (K12481), ab47+c122 (K12482), ab47+c123 (K12483), ab47+c124 (K12484), ab47+c125 (K12485), ab47+c126 (K12486), ab47+c127 (K12487), ab47+c128 (K12488), ab47+c129 (K12489), ab47+c130 (K12490), ab47+c131 (K12491), ab47+c132 (K12492), ab47+c133 (K12493), ab47+c134 (K12494), ab47+c135 (K12495), ab47+c136 (K12496), ab47+c137 (K12497), ab47+c138 (K12498), ab47+c139 (K12499), ab47+c140 (K12500), ab47+c141 (K12501), ab47+c142 (K12502), ab47+c143 (K12503), ab47+c144 (K12504), ab47+c145 (K12505), ab47+c146 (K12506), ab47+c147 (K12507), ab47+c148 (K12508), ab47+c149 (K12509), ab47+c150 (K12510), ab47+c151 (K12511), ab47+c152 (K12512), ab47+c153 (K12513), ab47+c154 (K12514), ab47+c155 (K12515), ab47+c156 (K12516), ab47+c157 (K12517), ab47+c158 (K12518), ab47+c159 (K12519), ab47+c160 (K12520), ab47+c161 (K12521), ab47+c162 (K12522), ab47+c163 (K12523), ab47+c164 (K12524), ab47+c165 (K12525), ab47+c166 (K12526), ab47+c167 (K12527), ab47+c168 (K12528), ab47+c169 (K12529), ab47+c170 (K12530), ab47+c171 (K12531), ab47+c172 (K12532), ab47+c173 (K12533), ab47+c174 (K12534), ab47+c175 (K12535), ab47+c176 (K12536), ab47+c177 (K12537), ab47+c178 (K12538), ab47+c179 (K12539), ab47+c180 (K12540), ab47+c181 (K12541), ab47+c182 (K12542), ab47+c183 (K12543), ab47+c184 (K12544), ab47+c185 (K12545), ab47+c186 (K12546), ab47+c187 (K12547), ab47+c188 (K12548), ab47+c189 (K12549), ab47+c190 (K12550), ab47+c191 (K12551), ab47+c192 (K12552), ab47+c193 (K12553), ab47+c194 (K12554), ab47+c195 (K12555), ab47+c196 (K12556), ab47+c197 (K12557), ab47+c198 (K12558), ab47+c199 (K12559), ab47+c200 (K12560), ab48+c1 (K12561), ab48+c2 (K12562), ab48+c3 (K12563), ab48+c4 (K12564), ab48+c5 (K12565), ab48+c6 (K12566), ab48+c7 (K12567), ab48+c8 (K12568), ab48+c9 (K12569), ab48+c10 (K12570), ab48+c11 (K12571), ab48+c12 (K12572), ab48+c13 (K12573), ab48+c14 (K12574), ab48+c15 (K12575), ab48+c16 (K12576), ab48+c17 (K12577), ab48+c18 (K12578), ab48+c19 (K12579), ab48+c20 (K12580), ab48+c21 (K12581), ab48+c22 (K12582), ab48+c23 (K12583), ab48+c24 (K12584), ab48+c25 (K12585), ab48+c26 (K12586), ab48+c27 (K12587), ab48+c28 (K12588), ab48+c29 (K12589), ab48+c30 (K12590), ab48+c31 (K12591), ab48+c32 (K12592), ab48+c33 (K12593), ab48+c34 (K12594), ab48+c35 (K12595), ab48+c36 (K12596), ab48+c37 (K12597), ab48+c38 (K12598), ab48+c39 (K12599), ab48+c40 (K12600), ab48+c41 (K12601), ab48+c42 (K12602), ab48+c43 (K12603), ab48+c44 (K12604), ab48+c45 (K12605), ab48+c46 (K12606), ab48+c47 (K12607), ab48+c48 (K12608), ab48+c49 (K12609), ab48+c50 (K12610), ab48+c51 (K12611), ab48+c52 (K12612), ab48+c53 (K12613), ab48+c54 (K12614), ab48+c55 (K12615), ab48+c56 (K12616), ab48+c57 (K12617), ab48+c58 (K12618), ab48+c59 (K12619), ab48+c60 (K12620), ab48+c61 (K12621), ab48+c62 (K12622), ab48+c63 (K12623), ab48+c64 (K12624), ab48+c65 (K12625), ab48+c66 (K12626), ab48+c67 (K12627), ab48+c68 (K12628), ab48+c69 (K12629), ab48+c70 (K12630), ab48+c71 (K12631), ab48+c72 (K12632), ab48+c73 (K12633), ab48+c74 (K12634), ab48+c75 (K12635), ab48+c76 (K12636), ab48+c77 (K12637), ab48+c78 (K12638), ab48+c79 (K12639), ab48+c80 (K12640), ab48+c81 (K12641), ab48+c82 (K12642), ab48+c83 (K12643), ab48+c84 (K12644), ab48+c85 (K12645), ab48+c86 (K12646), ab48+c87 (K12647), ab48+c88 (K12648), ab48+c89 (K12649), ab48+c90 (K12650), ab48+c91 (K12651), ab48+c92 (K12652), ab48+c93 (K12653), ab48+c94 (K12654), ab48+c95 (K12655), ab48+c96 (K12656), ab48+c97 (K12657), ab48+c98 (K12658), ab48+c99 (K12659), ab48+c100 (K12660), ab48+c101 (K12661), ab48+c102 (K12662), ab48+c103 (K12663), ab48+c104 (K12664), ab48+c105 (K12665), ab48+c106 (K12666), ab48+c107 (K12667), ab48+c108 (K12668), ab48+c109 (K12669), ab48+c110 (K12670), ab48+c111 (K12671), ab48+c112 (K12672), ab48+c113 (K12673), ab48+c114 (K12674), ab48+c115 (K12675), ab48+c116 (K12676), ab48+c117 (K12677), ab48+c118 (K12678), ab48+c119 (K12679), ab48+c120 (K12680), ab48+c121 (K12681), ab48+c122 (K12682), ab48+c123 (K12683), ab48+c124 (K12684), ab48+c125 (K12685), ab48+c126 (K12686), ab48+c127 (K12687), ab48+c128 (K12688), ab48+c129 (K12689), ab48+c130 (K12690), ab48+c131 (K12691), ab48+c132 (K12692), ab48+c133 (K12693), ab48+c134 (K12694), ab48+c135 (K12695), ab48+c136 (K12696), ab48+c137 (K12697), ab48+c138 (K12698), ab48+c139 (K12699), ab48+c140 (K12700), ab48+c141 (K12701), ab48+c142 (K12702), ab48+c143 (K12703), ab48+c144 (K12704), ab48+c145 (K12705), ab48+c146 (K12706), ab48+c147 (K12707), ab48+c148 (K12708), ab48+c149 (K12709), ab48+c150 (K12710), ab48+c151 (K12711), ab48+c152 (K12712), ab48+c153 (K12713), ab48+c154 (K12714), ab48+c155 (K12715), ab48+c156 (K12716), ab48+c157 (K12717), ab48+c158 (K12718), ab48+c159 (K12719), ab48+c160 (K12720), ab48+c161 (K12721), ab48+c162 (K12722), ab48+c163 (K12723), ab48+c164 (K12724), ab48+c165 (K12725), ab48+c166 (K12726), ab48+c167 (K12727), ab48+c168 (K12728), ab48+c169 (K12729), ab48+c170 (K12730), ab48+c171 (K12731), ab48+c172 (K12732), ab48+c173 (K12733), ab48+c174 (K12734), ab48+c175 (K12735), ab48+c176 (K12736), ab48+c177 (K12737), ab48+c178 (K12738), ab48+c179 (K12739), ab48+c180 (K12740), ab48+c181 (K12741), ab48+c182 (K12742), ab48+c183 (K12743), ab48+c184 (K12744), ab48+c185 (K12745), ab48+c186 (K12746), ab48+c187 (K12747), ab48+c188 (K12748), ab48+c189 (K12749), ab48+c190 (K12750), ab48+c191 (K12751), ab48+c192 (K12752), ab48+c193 (K12753), ab48+c194 (K12754), ab48+c195 (K12755), ab48+c196 (K12756), ab48+c197 (K12757), ab48+c198 (K12758), ab48+c199 (K12759), ab48+c200 (K12760), ab49+c1 (K12761), ab49+c2 (K12762), ab49+c3 (K12763), ab49+c4 (K12764), ab49+c5 (K12765), ab49+c6 (K12766), ab49+c7 (K12767), ab49+c8 (K12768), ab49+c9 (K12769), ab49+c10 (K12770), ab49+c11 (K12771), ab49+c12 (K12772), ab49+c13 (K12773), ab49+c14 (K12774), ab49+c15 (K12775), ab49+c16 (K12776), ab49+c17 (K12777), ab49+c18 (K12778), ab49+c19 (K12779), ab49+c20 (K12780), ab49+c21 (K12781), ab49+c22 (K12782), ab49+c23 (K12783), ab49+c24 (K12784), ab49+c25 (K12785), ab49+c26 (K12786), ab49+c27 (K12787), ab49+c28 (K12788), ab49+c29 (K12789), ab49+c30 (K12790), ab49+c31 (K12791), ab49+c32 (K12792), ab49+c33 (K12793), ab49+c34 (K12794), ab49+c35 (K12795), ab49+c36 (K12796), ab49+c37 (K12797), ab49+c38 (K12798), ab49+c39 (K12799), ab49+c40 (K12800), ab49+c41 (K12801), ab49+c42 (K12802), ab49+c43 (K12803), ab49+c44 (K12804), ab49+c45 (K12805), ab49+c46 (K12806), ab49+c47 (K12807), ab49+c48 (K12808), ab49+c49 (K12809), ab49+c50 (K12810), ab49+c51 (K12811), ab49+c52 (K12812), ab49+c53 (K12813), ab49+c54 (K12814), ab49+c55 (K12815), ab49+c56 (K12816), ab49+c57 (K12817), ab49+c58 (K12818), ab49+c59 (K12819), ab49+c60 (K12820), ab49+c61 (K12821), ab49+c62 (K12822), ab49+c63 (K12823), ab49+c64 (K12824), ab49+c65 (K12825), ab49+c66 (K12826), ab49+c67 (K12827), ab49+c68 (K12828), ab49+c69 (K12829), ab49+c70 (K12830), ab49+c71 (K12831), ab49+c72 (K12832), ab49+c73 (K12833), ab49+c74 (K12834), ab49+c75 (K12835), ab49+c76 (K12836), ab49+c77 (K12837), ab49+c78 (K12838), ab49+c79 (K12839), ab49+c80 (K12840), ab49+c81 (K12841), ab49+c82 (K12842), ab49+c83 (K12843), ab49+c84 (K12844), ab49+c85 (K12845), ab49+c86 (K12846), ab49+c87 (K12847), ab49+c88 (K12848), ab49+c89 (K12849), ab49+c90 (K12850), ab49+c91 (K12851), ab49+c92 (K12852), ab49+c93 (K12853), ab49+c94 (K12854), ab49+c95 (K12855), ab49+c96 (K12856), ab49+c97 (K12857), ab49+c98 (K12858), ab49+c99 (K12859), ab49+c100 (K12860), ab49+c101 (K12861), ab49+c102 (K12862), ab49+c103 (K12863), ab49+c104 (K12864), ab49+c105 (K12865), ab49+c106 (K12866), ab49+c107 (K12867), ab49+c108 (K12868), ab49+c109 (K12869), ab49+c110 (K12870), ab49+c111 (K12871), ab49+c112 (K12872), ab49+c113 (K12873), ab49+c114 (K12874), ab49+c115 (K12875), ab49+c116 (K12876), ab49+c117 (K12877), ab49+c118 (K12878), ab49+c119 (K12879), ab49+c120 (K12880), ab49+c121 (K12881), ab49+c122 (K12882), ab49+c123 (K12883), ab49+c124 (K12884), ab49+c125 (K12885), ab49+c126 (K12886), ab49+c127 (K12887), ab49+c128 (K12888), ab49+c129 (K12889), ab49+c130 (K12890), ab49+c131 (K12891), ab49+c132 (K12892), ab49+c133 (K12893), ab49+c134 (K12894), ab49+c135 (K12895), ab49+c136 (K12896), ab49+c137 (K12897), ab49+c138 (K12898), ab49+c139 (K12899), ab49+c140 (K12900), ab49+c141 (K12901), ab49+c142 (K12902), ab49+c143 (K12903), ab49+c144 (K12904), ab49+c145 (K12905), ab49+c146 (K12906), ab49+c147 (K12907), ab49+c148 (K12908), ab49+c149 (K12909), ab49+c150 (K12910), ab49+c151 (K12911), ab49+c152 (K12912), ab49+c153 (K12913), ab49+c154 (K12914), ab49+c155 (K12915), ab49+c156 (K12916), ab49+c157 (K12917), ab49+c158 (K12918), ab49+c159 (K12919), ab49+c160 (K12920), ab49+c161 (K12921), ab49+c162 (K12922), ab49+c163 (K12923), ab49+c164 (K12924), ab49+c165 (K12925), ab49+c166 (K12926), ab49+c167 (K12927), ab49+c168 (K12928), ab49+c169 (K12929), ab49+c170 (K12930), ab49+c171 (K12931), ab49+c172 (K12932), ab49+c173 (K12933), ab49+c174 (K12934), ab49+c175 (K12935), ab49+c176 (K12936), ab49+c177 (K12937), ab49+c178 (K12938), ab49+c179 (K12939), ab49+c180 (K12940), ab49+c181 (K12941), ab49+c182 (K12942), ab49+c183 (K12943), ab49+c184 (K12944), ab49+c185 (K12945), ab49+c186 (K12946), ab49+c187 (K12947), ab49+c188 (K12948), ab49+c189 (K12949), ab49+c190 (K12950), ab49+c191 (K12951), ab49+c192 (K12952), ab49+c193 (K12953), ab49+c194 (K12954), ab49+c195 (K12955), ab49+c196 (K12956), ab49+c197 (K12957), ab49+c198 (K12958), ab49+c199 (K12959), ab49+c200 (K12960), ab50+c1 (K12961), ab50+c2 (K12962), ab50+c3 (K12963), ab50+c4 (K12964), ab50+c5 (K12965), ab50+c6 (K12966), ab50+c7 (K12967), ab50+c8 (K12968), ab50+c9 (K12969), ab50+c10 (K12970), ab50+c11 (K12971), ab50+c12 (K12972), ab50+c13 (K12973), ab50+c14 (K12974), ab50+c15 (K12975), ab50+c16 (K12976), ab50+c17 (K12977), ab50+c18 (K12978), ab50+c19 (K12979), ab50+c20 (K12980), ab50+c21 (K12981), ab50+c22 (K12982), ab50+c23 (K12983), ab50+c24 (K12984), ab50+c25 (K12985), ab50+c26 (K12986), ab50+c27 (K12987), ab50+c28 (K12988), ab50+c29 (K12989), ab50+c30 (K12990), ab50+c31 (K12991), ab50+c32 (K12992), ab50+c33 (K12993), ab50+c34 (K12994), ab50+c35 (K12995), ab50+c36 (K12996), ab50+c37 (K12997), ab50+c38 (K12998), ab50+c39 (K12999), ab50+c40 (K13000), ab50+c41 (K13001), ab50+c42 (K13002), ab50+c43 (K13003), ab50+c44 (K13004), ab50+c45 (K13005), ab50+c46 (K13006), ab50+c47 (K13007), ab50+c48 (K13008), ab50+c49 (K13009), ab50+c50 (K13010), ab50+c51 (K13011), ab50+c52 (K13012), ab50+c53 (K13013), ab50+c54 (K13014), ab50+c55 (K13015), ab50+c56 (K13016), ab50+c57 (K13017), ab50+c58 (K13018), ab50+c59 (K13019), ab50+c60 (K13020), ab50+c61 (K13021), ab50+c62 (K13022), ab50+c63 (K13023), ab50+c64 (K13024), ab50+c65 (K13025), ab50+c66 (K13026), ab50+c67 (K13027), ab50+c68 (K13028), ab50+c69 (K13029), ab50+c70 (K13030), ab50+c71 (K13031), ab50+c72 (K13032), ab50+c73 (K13033), ab50+c74 (K13034), ab50+c75 (K13035), ab50+c76 (K13036), ab50+c77 (K13037), ab50+c78 (K13038), ab50+c79 (K13039), ab50+c80 (K13040), ab50+c81 (K13041), ab50+c82 (K13042), ab50+c83 (K13043), ab50+c84 (K13044), ab50+c85 (K13045), ab50+c86 (K13046), ab50+c87 (K13047), ab50+c88 (K13048), ab50+c89 (K13049), ab50+c90 (K13050), ab50+c91 (K13051), ab50+c92 (K13052), ab50+c93 (K13053), ab50+c94 (K13054), ab50+c95 (K13055), ab50+c96 (K13056), ab50+c97 (K13057), ab50+c98 (K13058), ab50+c99 (K13059), ab50+c100 (K13060), ab50+c101 (K13061), ab50+c102 (K13062), ab50+c103 (K13063), ab50+c104 (K13064), ab50+c105 (K13065), ab50+c106 (K13066), ab50+c107 (K13067), ab50+c108 (K13068), ab50+c109 (K13069), ab50+c110 (K13070), ab50+c111 (K13071), ab50+c112 (K13072), ab50+c113 (K13073), ab50+c114 (K13074), ab50+c115 (K13075), ab50+c116 (K13076), ab50+c117 (K13077), ab50+c118 (K13078), ab50+c119 (K13079), ab50+c120 (K13080), ab50+c121 (K13081), ab50+c122 (K13082), ab50+c123 (K13083), ab50+c124 (K13084), ab50+c125 (K13085), ab50+c126 (K13086), ab50+c127 (K13087), ab50+c128 (K13088), ab50+c129 (K13089), ab50+c130 (K13090), ab50+c131 (K13091), ab50+c132 (K13092), ab50+c133 (K13093), ab50+c134 (K13094), ab50+c135 (K13095), ab50+c136 (K13096), ab50+c137 (K13097), ab50+c138 (K13098), ab50+c139 (K13099), ab50+c140 (K13100), ab50+c141 (K13101), ab50+c142 (K13102), ab50+c143 (K13103), ab50+c144 (K13104), ab50+c145 (K13105), ab50+c146 (K13106), ab50+c147 (K13107), ab50+c148 (K13108), ab50+c149 (K13109), ab50+c150 (K13110), ab50+c151 (K13111), ab50+c152 (K13112), ab50+c153 (K13113), ab50+c154 (K13114), ab50+c155 (K13115), ab50+c156 (K13116), ab50+c157 (K13117), ab50+c158 (K13118), ab50+c159 (K13119), ab50+c160 (K13120), ab50+c161 (K13121), ab50+c162 (K13122), ab50+c163 (K13123), ab50+c164 (K13124), ab50+c165 (K13125), ab50+c166 (K13126), ab50+c167 (K13127), ab50+c168 (K13128), ab50+c169 (K13129), ab50+c170 (K13130), ab50+c171 (K13131), ab50+c172 (K13132), ab50+c173 (K13133), ab50+c174 (K13134), ab50+c175 (K13135), ab50+c176 (K13136), ab50+c177 (K13137), ab50+c178 (K13138), ab50+c179 (K13139), ab50+c180 (K13140), ab50+c181 (K13141), ab50+c182 (K13142), ab50+c183 (K13143), ab50+c184 (K13144), ab50+c185 (K13145), ab50+c186 (K13146), ab50+c187 (K13147), ab50+c188 (K13148), ab50+c189 (K13149), ab50+c190 (K13150), ab50+c191 (K13151), ab50+c192 (K13152), ab50+c193 (K13153), ab50+c194 (K13154), ab50+c195 (K13155), ab50+c196 (K13156), ab50+c197 (K13157), ab50+c198 (K13158), ab50+c199 (K13159), ab50+c200 (K13160), ab51+c1 (K13161), ab51+c2 (K13162), ab51+c3 (K13163), ab51+c4 (K13164), ab51+c5 (K13165), ab51+c6 (K13166), ab51+c7 (K13167), ab51+c8 (K13168), ab51+c9 (K13169), ab51+c10 (K13170), ab51+c11 (K13171), ab51+c12 (K13172), ab51+c13 (K13173), ab51+c14 (K13174), ab51+c15 (K13175), ab51+c16 (K13176), ab51+c17 (K13177), ab51+c18 (K13178), ab51+c19 (K13179), ab51+c20 (K13180), ab51+c21 (K13181), ab51+c22 (K13182), ab51+c23 (K13183), ab51+c24 (K13184), ab51+c25 (K13185), ab51+c26 (K13186), ab51+c27 (K13187), ab51+c28 (K13188), ab51+c29 (K13189), ab51+c30 (K13190), ab51+c31 (K13191), ab51+c32 (K13192), ab51+c33 (K13193), ab51+c34 (K13194), ab51+c35 (K13195), ab51+c36 (K13196), ab51+c37 (K13197), ab51+c38 (K13198), ab51+c39 (K13199), ab51+c40 (K13200), ab51+c41 (K13201), ab51+c42 (K13202), ab51+c43 (K13203), ab51+c44 (K13204), ab51+c45 (K13205), ab51+c46 (K13206), ab51+c47 (K13207), ab51+c48 (K13208), ab51+c49 (K13209), ab51+c50 (K13210), ab51+c51 (K13211), ab51+c52 (K13212), ab51+c53 (K13213), ab51+c54 (K13214), ab51+c55 (K13215), ab51+c56 (K13216), ab51+c57 (K13217), ab51+c58 (K13218), ab51+c59 (K13219), ab51+c60 (K13220), ab51+c61 (K13221), ab51+c62 (K13222), ab51+c63 (K13223), ab51+c64 (K13224), ab51+c65 (K13225), ab51+c66 (K13226), ab51+c67 (K13227), ab51+c68 (K13228), ab51+c69 (K13229), ab51+c70 (K13230), ab51+c71 (K13231), ab51+c72 (K13232), ab51+c73 (K13233), ab51+c74 (K13234), ab51+c75 (K13235), ab51+c76 (K13236), ab51+c77 (K13237), ab51+c78 (K13238), ab51+c79 (K13239), ab51+c80 (K13240), ab51+c81 (K13241), ab51+c82 (K13242), ab51+c83 (K13243), ab51+c84 (K13244), ab51+c85 (K13245), ab51+c86 (K13246), ab51+c87 (K13247), ab51+c88 (K13248), ab51+c89 (K13249), ab51+c90 (K13250), ab51+c91 (K13251), ab51+c92 (K13252), ab51+c93 (K13253), ab51+c94 (K13254), ab51+c95 (K13255), ab51+c96 (K13256), ab51+c97 (K13257), ab51+c98 (K13258), ab51+c99 (K13259), ab51+c100 (K13260), ab51+c101 (K13261), ab51+c102 (K13262), ab51+c103 (K13263), ab51+c104 (K13264), ab51+c105 (K13265), ab51+c106 (K13266), ab51+c107 (K13267), ab51+c108 (K13268), ab51+c109 (K13269), ab51+c110 (K13270), ab51+c111 (K13271), ab51+c112 (K13272), ab51+c113 (K13273), ab51+c114 (K13274), ab51+c115 (K13275), ab51+c116 (K13276), ab51+c117 (K13277), ab51+c118 (K13278), ab51+c119 (K13279), ab51+c120 (K13280), ab51+c121 (K13281), ab51+c122 (K13282), ab51+c123 (K13283), ab51+c124 (K13284), ab51+c125 (K13285), ab51+c126 (K13286), ab51+c127 (K13287), ab51+c128 (K13288), ab51+c129 (K13289), ab51+c130 (K13290), ab51+c131 (K13291), ab51+c132 (K13292), ab51+c133 (K13293), ab51+c134 (K13294), ab51+c135 (K13295), ab51+c136 (K13296), ab51+c137 (K13297), ab51+c138 (K13298), ab51+c139 (K13299), ab51+c140 (K13300), ab51+c141 (K13301), ab51+c142 (K13302), ab51+c143 (K13303), ab51+c144 (K13304), ab51+c145 (K13305), ab51+c146 (K13306), ab51+c147 (K13307), ab51+c148 (K13308), ab51+c149 (K13309), ab51+c150 (K13310), ab51+c151 (K13311), ab51+c152 (K13312), ab51+c153 (K13313), ab51+c154 (K13314), ab51+c155 (K13315), ab51+c156 (K13316), ab51+c157 (K13317), ab51+c158 (K13318), ab51+c159 (K13319), ab51+c160 (K13320), ab51+c161 (K13321), ab51+c162 (K13322), ab51+c163 (K13323), ab51+c164 (K13324), ab51+c165 (K13325), ab51+c166 (K13326), ab51+c167 (K13327), ab51+c168 (K13328), ab51+c169 (K13329), ab51+c170 (K13330), ab51+c171 (K13331), ab51+c172 (K13332), ab51+c173 (K13333), ab51+c174 (K13334), ab51+c175 (K13335), ab51+c176 (K13336), ab51+c177 (K13337), ab51+c178 (K13338), ab51+c179 (K13339), ab51+c180 (K13340), ab51+c181 (K13341), ab51+c182 (K13342), ab51+c183 (K13343), ab51+c184 (K13344), ab51+c185 (K13345), ab51+c186 (K13346), ab51+c187 (K13347), ab51+c188 (K13348), ab51+c189 (K13349), ab51+c190 (K13350), ab51+c191 (K13351), ab51+c192 (K13352), ab51+c193 (K13353), ab51+c194 (K13354), ab51+c195 (K13355), ab51+c196 (K13356), ab51+c197 (K13357), ab51+c198 (K13358), ab51+c199 (K13359), ab51+c200 (K13360), ab52+c1 (K13361), ab52+c2 (K13362), ab52+c3 (K13363), ab52+c4 (K13364), ab52+c5 (K13365), ab52+c6 (K13366), ab52+c7 (K13367), ab52+c8 (K13368), ab52+c9 (K13369), ab52+c10 (K13370), ab52+c11 (K13371), ab52+c12 (K13372), ab52+c13 (K13373), ab52+c14 (K13374), ab52+c15 (K13375), ab52+c16 (K13376), ab52+c17 (K13377), ab52+c18 (K13378), ab52+c19 (K13379), ab52+c20 (K13380), ab52+c21 (K13381), ab52+c22 (K13382), ab52+c23 (K13383), ab52+c24 (K13384), ab52+c25 (K13385), ab52+c26 (K13386), ab52+c27 (K13387), ab52+c28 (K13388), ab52+c29 (K13389), ab52+c30 (K13390), ab52+c31 (K13391), ab52+c32 (K13392), ab52+c33 (K13393), ab52+c34 (K13394), ab52+c35 (K13395), ab52+c36 (K13396), ab52+c37 (K13397), ab52+c38 (K13398), ab52+c39 (K13399), ab52+c40 (K13400), ab52+c41 (K13401), ab52+c42 (K13402), ab52+c43 (K13403), ab52+c44 (K13404), ab52+c45 (K13405), ab52+c46 (K13406), ab52+c47 (K13407), ab52+c48 (K13408), ab52+c49 (K13409), ab52+c50 (K13410), ab52+c51 (K13411), ab52+c52 (K13412), ab52+c53 (K13413), ab52+c54 (K13414), ab52+c55 (K13415), ab52+c56 (K13416), ab52+c57 (K13417), ab52+c58 (K13418), ab52+c59 (K13419), ab52+c60 (K13420), ab52+c61 (K13421), ab52+c62 (K13422), ab52+c63 (K13423), ab52+c64 (K13424), ab52+c65 (K13425), ab52+c66 (K13426), ab52+c67 (K13427), ab52+c68 (K13428), ab52+c69 (K13429), ab52+c70 (K13430), ab52+c71 (K13431), ab52+c72 (K13432), ab52+c73 (K13433), ab52+c74 (K13434), ab52+c75 (K13435), ab52+c76 (K13436), ab52+c77 (K13437), ab52+c78 (K13438), ab52+c79 (K13439), ab52+c80 (K13440), ab52+c81 (K13441), ab52+c82 (K13442), ab52+c83 (K13443), ab52+c84 (K13444), ab52+c85 (K13445), ab52+c86 (K13446), ab52+c87 (K13447), ab52+c88 (K13448), ab52+c89 (K13449), ab52+c90 (K13450), ab52+c91 (K13451), ab52+c92 (K13452), ab52+c93 (K13453), ab52+c94 (K13454), ab52+c95 (K13455), ab52+c96 (K13456), ab52+c97 (K13457), ab52+c98 (K13458), ab52+c99 (K13459), ab52+c100 (K13460), ab52+c101 (K13461), ab52+c102 (K13462), ab52+c103 (K13463), ab52+c104 (K13464), ab52+c105 (K13465), ab52+c106 (K13466), ab52+c107 (K13467), ab52+c108 (K13468), ab52+c109 (K13469), ab52+c110 (K13470), ab52+c111 (K13471), ab52+c112 (K13472), ab52+c113 (K13473), ab52+c114 (K13474), ab52+c115 (K13475), ab52+c116 (K13476), ab52+c117 (K13477), ab52+c118 (K13478), ab52+c119 (K13479), ab52+c120 (K13480), ab52+c121 (K13481), ab52+c122 (K13482), ab52+c123 (K13483), ab52+c124 (K13484), ab52+c125 (K13485), ab52+c126 (K13486), ab52+c127 (K13487), ab52+c128 (K13488), ab52+c129 (K13489), ab52+c130 (K13490), ab52+c131 (K13491), ab52+c132 (K13492), ab52+c133 (K13493), ab52+c134 (K13494), ab52+c135 (K13495), ab52+c136 (K13496), ab52+c137 (K13497), ab52+c138 (K13498), ab52+c139 (K13499), ab52+c140 (K13500), ab52+c141 (K13501), ab52+c142 (K13502), ab52+c143 (K13503), ab52+c144 (K13504), ab52+c145 (K13505), ab52+c146 (K13506), ab52+c147 (K13507), ab52+c148 (K13508), ab52+c149 (K13509), ab52+c150 (K13510), ab52+c151 (K13511), ab52+c152 (K13512), ab52+c153 (K13513), ab52+c154 (K13514), ab52+c155 (K13515), ab52+c156 (K13516), ab52+c157 (K13517), ab52+c158 (K13518), ab52+c159 (K13519), ab52+c160 (K13520), ab52+c161 (K13521), ab52+c162 (K13522), ab52+c163 (K13523), ab52+c164 (K13524), ab52+c165 (K13525), ab52+c166 (K13526), ab52+c167 (K13527), ab52+c168 (K13528), ab52+c169 (K13529), ab52+c170 (K13530), ab52+c171 (K13531), ab52+c172 (K13532), ab52+c173 (K13533), ab52+c174 (K13534), ab52+c175 (K13535), ab52+c176 (K13536), ab52+c177 (K13537), ab52+c178 (K13538), ab52+c179 (K13539), ab52+c180 (K13540), ab52+c181 (K13541), ab52+c182 (K13542), ab52+c183 (K13543), ab52+c184 (K13544), ab52+c185 (K13545), ab52+c186 (K13546), ab52+c187 (K13547), ab52+c188 (K13548), ab52+c189 (K13549), ab52+c190 (K13550), ab52+c191 (K13551), ab52+c192 (K13552), ab52+c193 (K13553), ab52+c194 (K13554), ab52+c195 (K13555), ab52+c196 (K13556), ab52+c197 (K13557), ab52+c198 (K13558), ab52+c199 (K13559), ab52+c200 (K13560), ab53+c1 (K13561), ab53+c2 (K13562), ab53+c3 (K13563), ab53+c4 (K13564), ab53+c5 (K13565), ab53+c6 (K13566), ab53+c7 (K13567), ab53+c8 (K13568), ab53+c9 (K13569), ab53+c10 (K13570), ab53+c11 (K13571), ab53+c12 (K13572), ab53+c13 (K13573), ab53+c14 (K13574), ab53+c15 (K13575), ab53+c16 (K13576), ab53+c17 (K13577), ab53+c18 (K13578), ab53+c19 (K13579), ab53+c20 (K13580), ab53+c21 (K13581), ab53+c22 (K13582), ab53+c23 (K13583), ab53+c24 (K13584), ab53+c25 (K13585), ab53+c26 (K13586), ab53+c27 (K13587), ab53+c28 (K13588), ab53+c29 (K13589), ab53+c30 (K13590), ab53+c31 (K13591), ab53+c32 (K13592), ab53+c33 (K13593), ab53+c34 (K13594), ab53+c35 (K13595), ab53+c36 (K13596), ab53+c37 (K13597), ab53+c38 (K13598), ab53+c39 (K13599), ab53+c40 (K13600), ab53+c41 (K13601), ab53+c42 (K13602), ab53+c43 (K13603), ab53+c44 (K13604), ab53+c45 (K13605), ab53+c46 (K13606), ab53+c47 (K13607), ab53+c48 (K13608), ab53+c49 (K13609), ab53+c50 (K13610), ab53+c51 (K13611), ab53+c52 (K13612), ab53+c53 (K13613), ab53+c54 (K13614), ab53+c55 (K13615), ab53+c56 (K13616), ab53+c57 (K13617), ab53+c58 (K13618), ab53+c59 (K13619), ab53+c60 (K13620), ab53+c61 (K13621), ab53+c62 (K13622), ab53+c63 (K13623), ab53+c64 (K13624), ab53+c65 (K13625), ab53+c66 (K13626), ab53+c67 (K13627), ab53+c68 (K13628), ab53+c69 (K13629), ab53+c70 (K13630), ab53+c71 (K13631), ab53+c72 (K13632), ab53+c73 (K13633), ab53+c74 (K13634), ab53+c75 (K13635), ab53+c76 (K13636), ab53+c77 (K13637), ab53+c78 (K13638), ab53+c79 (K13639), ab53+c80 (K13640), ab53+c81 (K13641), ab53+c82 (K13642), ab53+c83 (K13643), ab53+c84 (K13644), ab53+c85 (K13645), ab53+c86 (K13646), ab53+c87 (K13647), ab53+c88 (K13648), ab53+c89 (K13649), ab53+c90 (K13650), ab53+c91 (K13651), ab53+c92 (K13652), ab53+c93 (K13653), ab53+c94 (K13654), ab53+c95 (K13655), ab53+c96 (K13656), ab53+c97 (K13657), ab53+c98 (K13658), ab53+c99 (K13659), ab53+c100 (K13660), ab53+c101 (K13661), ab53+c102 (K13662), ab53+c103 (K13663), ab53+c104 (K13664), ab53+c105 (K13665), ab53+c106 (K13666), ab53+c107 (K13667), ab53+c108 (K13668), ab53+c109 (K13669), ab53+c110 (K13670), ab53+c111 (K13671), ab53+c112 (K13672), ab53+c113 (K13673), ab53+c114 (K13674), ab53+c115 (K13675), ab53+c116 (K13676), ab53+c117 (K13677), ab53+c118 (K13678), ab53+c119 (K13679), ab53+c120 (K13680), ab53+c121 (K13681), ab53+c122 (K13682), ab53+c123 (K13683), ab53+c124 (K13684), ab53+c125 (K13685), ab53+c126 (K13686), ab53+c127 (K13687), ab53+c128 (K13688), ab53+c129 (K13689), ab53+c130 (K13690), ab53+c131 (K13691), ab53+c132 (K13692), ab53+c133 (K13693), ab53+c134 (K13694), ab53+c135 (K13695), ab53+c136 (K13696), ab53+c137 (K13697), ab53+c138 (K13698), ab53+c139 (K13699), ab53+c140 (K13700), ab53+c141 (K13701), ab53+c142 (K13702), ab53+c143 (K13703), ab53+c144 (K13704), ab53+c145 (K13705), ab53+c146 (K13706), ab53+c147 (K13707), ab53+c148 (K13708), ab53+c149 (K13709), ab53+c150 (K13710), ab53+c151 (K13711), ab53+c152 (K13712), ab53+c153 (K13713), ab53+c154 (K13714), ab53+c155 (K13715), ab53+c156 (K13716), ab53+c157 (K13717), ab53+c158 (K13718), ab53+c159 (K13719), ab53+c160 (K13720), ab53+c161 (K13721), ab53+c162 (K13722), ab53+c163 (K13723), ab53+c164 (K13724), ab53+c165 (K13725), ab53+c166 (K13726), ab53+c167 (K13727), ab53+c168 (K13728), ab53+c169 (K13729), ab53+c170 (K13730), ab53+c171 (K13731), ab53+c172 (K13732), ab53+c173 (K13733), ab53+c174 (K13734), ab53+c175 (K13735), ab53+c176 (K13736), ab53+c177 (K13737), ab53+c178 (K13738), ab53+c179 (K13739), ab53+c180 (K13740), ab53+c181 (K13741), ab53+c182 (K13742), ab53+c183 (K13743), ab53+c184 (K13744), ab53+c185 (K13745), ab53+c186 (K13746), ab53+c187 (K13747), ab53+c188 (K13748), ab53+c189 (K13749), ab53+c190 (K13750), ab53+c191 (K13751), ab53+c192 (K13752), ab53+c193 (K13753), ab53+c194 (K13754), ab53+c195 (K13755), ab53+c196 (K13756), ab53+c197 (K13757), ab53+c198 (K13758), ab53+c199 (K13759), ab53+c200 (K13760), ab54+c1 (K13761), ab54+c2 (K13762), ab54+c3 (K13763), ab54+c4 (K13764), ab54+c5 (K13765), ab54+c6 (K13766), ab54+c7 (K13767), ab54+c8 (K13768), ab54+c9 (K13769), ab54+c10 (K13770), ab54+c11 (K13771), ab54+c12 (K13772), ab54+c13 (K13773), ab54+c14 (K13774), ab54+c15 (K13775), ab54+c16 (K13776), ab54+c17 (K13777), ab54+c18 (K13778), ab54+c19 (K13779), ab54+c20 (K13780), ab54+c21 (K13781), ab54+c22 (K13782), ab54+c23 (K13783), ab54+c24 (K13784), ab54+c25 (K13785), ab54+c26 (K13786), ab54+c27 (K13787), ab54+c28 (K13788), ab54+c29 (K13789), ab54+c30 (K13790), ab54+c31 (K13791), ab54+c32 (K13792), ab54+c33 (K13793), ab54+c34 (K13794), ab54+c35 (K13795), ab54+c36 (K13796), ab54+c37 (K13797), ab54+c38 (K13798), ab54+c39 (K13799), ab54+c40 (K13800), ab54+c41 (K13801), ab54+c42 (K13802), ab54+c43 (K13803), ab54+c44 (K13804), ab54+c45 (K13805), ab54+c46 (K13806), ab54+c47 (K13807), ab54+c48 (K13808), ab54+c49 (K13809), ab54+c50 (K13810), ab54+c51 (K13811), ab54+c52 (K13812), ab54+c53 (K13813), ab54+c54 (K13814), ab54+c55 (K13815), ab54+c56 (K13816), ab54+c57 (K13817), ab54+c58 (K13818), ab54+c59 (K13819), ab54+c60 (K13820), ab54+c61 (K13821), ab54+c62 (K13822), ab54+c63 (K13823), ab54+c64 (K13824), ab54+c65 (K13825), ab54+c66 (K13826), ab54+c67 (K13827), ab54+c68 (K13828), ab54+c69 (K13829), ab54+c70 (K13830), ab54+c71 (K13831), ab54+c72 (K13832), ab54+c73 (K13833), ab54+c74 (K13834), ab54+c75 (K13835), ab54+c76 (K13836), ab54+c77 (K13837), ab54+c78 (K13838), ab54+c79 (K13839), ab54+c80 (K13840), ab54+c81 (K13841), ab54+c82 (K13842), ab54+c83 (K13843), ab54+c84 (K13844), ab54+c85 (K13845), ab54+c86 (K13846), ab54+c87 (K13847), ab54+c88 (K13848), ab54+c89 (K13849), ab54+c90 (K13850), ab54+c91 (K13851), ab54+c92 (K13852), ab54+c93 (K13853), ab54+c94 (K13854), ab54+c95 (K13855), ab54+c96 (K13856), ab54+c97 (K13857), ab54+c98 (K13858), ab54+c99 (K13859), ab54+c100 (K13860), ab54+c101 (K13861), ab54+c102 (K13862), ab54+c103 (K13863), ab54+c104 (K13864), ab54+c105 (K13865), ab54+c106 (K13866), ab54+c107 (K13867), ab54+c108 (K13868), ab54+c109 (K13869), ab54+c110 (K13870), ab54+c111 (K13871), ab54+c112 (K13872), ab54+c113 (K13873), ab54+c114 (K13874), ab54+c115 (K13875), ab54+c116 (K13876), ab54+c117 (K13877), ab54+c118 (K13878), ab54+c119 (K13879), ab54+c120 (K13880), ab54+c121 (K13881), ab54+c122 (K13882), ab54+c123 (K13883), ab54+c124 (K13884), ab54+c125 (K13885), ab54+c126 (K13886), ab54+c127 (K13887), ab54+c128 (K13888), ab54+c129 (K13889), ab54+c130 (K13890), ab54+c131 (K13891), ab54+c132 (K13892), ab54+c133 (K13893), ab54+c134 (K13894), ab54+c135 (K13895), ab54+c136 (K13896), ab54+c137 (K13897), ab54+c138 (K13898), ab54+c139 (K13899), ab54+c140 (K13900), ab54+c141 (K13901), ab54+c142 (K13902), ab54+c143 (K13903), ab54+c144 (K13904), ab54+c145 (K13905), ab54+c146 (K13906), ab54+c147 (K13907), ab54+c148 (K13908), ab54+c149 (K13909), ab54+c150 (K13910), ab54+c151 (K13911), ab54+c152 (K13912), ab54+c153 (K13913), ab54+c154 (K13914), ab54+c155 (K13915), ab54+c156 (K13916), ab54+c157 (K13917), ab54+c158 (K13918), ab54+c159 (K13919), ab54+c160 (K13920), ab54+c161 (K13921), ab54+c162 (K13922), ab54+c163 (K13923), ab54+c164 (K13924), ab54+c165 (K13925), ab54+c166 (K13926), ab54+c167 (K13927), ab54+c168 (K13928), ab54+c169 (K13929), ab54+c170 (K13930), ab54+c171 (K13931), ab54+c172 (K13932), ab54+c173 (K13933), ab54+c174 (K13934), ab54+c175 (K13935), ab54+c176 (K13936), ab54+c177 (K13937), ab54+c178 (K13938), ab54+c179 (K13939), ab54+c180 (K13940), ab54+c181 (K13941), ab54+c182 (K13942), ab54+c183 (K13943), ab54+c184 (K13944), ab54+c185 (K13945), ab54+c186 (K13946), ab54+c187 (K13947), ab54+c188 (K13948), ab54+c189 (K13949), ab54+c190 (K13950), ab54+c191 (K13951), ab54+c192 (K13952), ab54+c193 (K13953), ab54+c194 (K13954), ab54+c195 (K13955), ab54+c196 (K13956), ab54+c197 (K13957), ab54+c198 (K13958), ab54+c199 (K13959), ab54+c200 (K13960), ab55+c1 (K13961), ab55+c2 (K13962), ab55+c3 (K13963), ab55+c4 (K13964), ab55+c5 (K13965), ab55+c6 (K13966), ab55+c7 (K13967), ab55+c8 (K13968), ab55+c9 (K13969), ab55+c10 (K13970), ab55+c11 (K13971), ab55+c12 (K13972), ab55+c13 (K13973), ab55+c14 (K13974), ab55+c15 (K13975), ab55+c16 (K13976), ab55+c17 (K13977), ab55+c18 (K13978), ab55+c19 (K13979), ab55+c20 (K13980), ab55+c21 (K13981), ab55+c22 (K13982), ab55+c23 (K13983), ab55+c24 (K13984), ab55+c25 (K13985), ab55+c26 (K13986), ab55+c27 (K13987), ab55+c28 (K13988), ab55+c29 (K13989), ab55+c30 (K13990), ab55+c31 (K13991), ab55+c32 (K13992), ab55+c33 (K13993), ab55+c34 (K13994), ab55+c35 (K13995), ab55+c36 (K13996), ab55+c37 (K13997), ab55+c38 (K13998), ab55+c39 (K13999), ab55+c40 (K14000), ab55+c41 (K14001), ab55+c42 (K14002), ab55+c43 (K14003), ab55+c44 (K14004), ab55+c45 (K14005), ab55+c46 (K14006), ab55+c47 (K14007), ab55+c48 (K14008), ab55+c49 (K14009), ab55+c50 (K14010), ab55+c51 (K14011), ab55+c52 (K14012), ab55+c53 (K14013), ab55+c54 (K14014), ab55+c55 (K14015), ab55+c56 (K14016), ab55+c57 (K14017), ab55+c58 (K14018), ab55+c59 (K14019), ab55+c60 (K14020), ab55+c61 (K14021), ab55+c62 (K14022), ab55+c63 (K14023), ab55+c64 (K14024), ab55+c65 (K14025), ab55+c66 (K14026), ab55+c67 (K14027), ab55+c68 (K14028), ab55+c69 (K14029), ab55+c70 (K14030), ab55+c71 (K14031), ab55+c72 (K14032), ab55+c73 (K14033), ab55+c74 (K14034), ab55+c75 (K14035), ab55+c76 (K14036), ab55+c77 (K14037), ab55+c78 (K14038), ab55+c79 (K14039), ab55+c80 (K14040), ab55+c81 (K14041), ab55+c82 (K14042), ab55+c83 (K14043), ab55+c84 (K14044), ab55+c85 (K14045), ab55+c86 (K14046), ab55+c87 (K14047), ab55+c88 (K14048), ab55+c89 (K14049), ab55+c90 (K14050), ab55+c91 (K14051), ab55+c92 (K14052), ab55+c93 (K14053), ab55+c94 (K14054), ab55+c95 (K14055), ab55+c96 (K14056), ab55+c97 (K14057), ab55+c98 (K14058), ab55+c99 (K14059), ab55+c100 (K14060), ab55+c101 (K14061), ab55+c102 (K14062), ab55+c103 (K14063), ab55+c104 (K14064), ab55+c105 (K14065), ab55+c106 (K14066), ab55+c107 (K14067), ab55+c108 (K14068), ab55+c109 (K14069), ab55+c110 (K14070), ab55+c111 (K14071), ab55+c112 (K14072), ab55+c113 (K14073), ab55+c114 (K14074), ab55+c115 (K14075), ab55+c116 (K14076), ab55+c117 (K14077), ab55+c118 (K14078), ab55+c119 (K14079), ab55+c120 (K14080), ab55+c121 (K14081), ab55+c122 (K14082), ab55+c123 (K14083), ab55+c124 (K14084), ab55+c125 (K14085), ab55+c126 (K14086), ab55+c127 (K14087), ab55+c128 (K14088), ab55+c129 (K14089), ab55+c130 (K14090), ab55+c131 (K14091), ab55+c132 (K14092), ab55+c133 (K14093), ab55+c134 (K14094), ab55+c135 (K14095), ab55+c136 (K14096), ab55+c137 (K14097), ab55+c138 (K14098), ab55+c139 (K14099), ab55+c140 (K14100), ab55+c141 (K14101), ab55+c142 (K14102), ab55+c143 (K14103), ab55+c144 (K14104), ab55+c145 (K14105), ab55+c146 (K14106), ab55+c147 (K14107), ab55+c148 (K14108), ab55+c149 (K14109), ab55+c150 (K14110), ab55+c151 (K14111), ab55+c152 (K14112), ab55+c153 (K14113), ab55+c154 (K14114), ab55+c155 (K14115), ab55+c156 (K14116), ab55+c157 (K14117), ab55+c158 (K14118), ab55+c159 (K14119), ab55+c160 (K14120), ab55+c161 (K14121), ab55+c162 (K14122), ab55+c163 (K14123), ab55+c164 (K14124), ab55+c165 (K14125), ab55+c166 (K14126), ab55+c167 (K14127), ab55+c168 (K14128), ab55+c169 (K14129), ab55+c170 (K14130), ab55+c171 (K14131), ab55+c172 (K14132), ab55+c173 (K14133), ab55+c174 (K14134), ab55+c175 (K14135), ab55+c176 (K14136), ab55+c177 (K14137), ab55+c178 (K14138), ab55+c179 (K14139), ab55+c180 (K14140), ab55+c181 (K14141), ab55+c182 (K14142), ab55+c183 (K14143), ab55+c184 (K14144), ab55+c185 (K14145), ab55+c186 (K14146), ab55+c187 (K14147), ab55+c188 (K14148), ab55+c189 (K14149), ab55+c190 (K14150), ab55+c191 (K14151), ab55+c192 (K14152), ab55+c193 (K14153), ab55+c194 (K14154), ab55+c195 (K14155), ab55+c196 (K14156), ab55+c197 (K14157), ab55+c198 (K14158), ab55+c199 (K14159), ab55+c200 (K14160), ab56+c1 (K14161), ab56+c2 (K14162), ab56+c3 (K14163), ab56+c4 (K14164), ab56+c5 (K14165), ab56+c6 (K14166), ab56+c7 (K14167), ab56+c8 (K14168), ab56+c9 (K14169), ab56+c10 (K14170), ab56+c11 (K14171), ab56+c12 (K14172), ab56+c13 (K14173), ab56+c14 (K14174), ab56+c15 (K14175), ab56+c16 (K14176), ab56+c17 (K14177), ab56+c18 (K14178), ab56+c19 (K14179), ab56+c20 (K14180), ab56+c21 (K14181), ab56+c22 (K14182), ab56+c23 (K14183), ab56+c24 (K14184), ab56+c25 (K14185), ab56+c26 (K14186), ab56+c27 (K14187), ab56+c28 (K14188), ab56+c29 (K14189), ab56+c30 (K14190), ab56+c31 (K14191), ab56+c32 (K14192), ab56+c33 (K14193), ab56+c34 (K14194), ab56+c35 (K14195), ab56+c36 (K14196), ab56+c37 (K14197), ab56+c38 (K14198), ab56+c39 (K14199), ab56+c40 (K14200), ab56+c41 (K14201), ab56+c42 (K14202), ab56+c43 (K14203), ab56+c44 (K14204), ab56+c45 (K14205), ab56+c46 (K14206), ab56+c47 (K14207), ab56+c48 (K14208), ab56+c49 (K14209), ab56+c50 (K14210), ab56+c51 (K14211), ab56+c52 (K14212), ab56+c53 (K14213), ab56+c54 (K14214), ab56+c55 (K14215), ab56+c56 (K14216), ab56+c57 (K14217), ab56+c58 (K14218), ab56+c59 (K14219), ab56+c60 (K14220), ab56+c61 (K14221), ab56+c62 (K14222), ab56+c63 (K14223), ab56+c64 (K14224), ab56+c65 (K14225), ab56+c66 (K14226), ab56+c67 (K14227), ab56+c68 (K14228), ab56+c69 (K14229), ab56+c70 (K14230), ab56+c71 (K14231), ab56+c72 (K14232), ab56+c73 (K14233), ab56+c74 (K14234), ab56+c75 (K14235), ab56+c76 (K14236), ab56+c77 (K14237), ab56+c78 (K14238), ab56+c79 (K14239), ab56+c80 (K14240), ab56+c81 (K14241), ab56+c82 (K14242), ab56+c83 (K14243), ab56+c84 (K14244), ab56+c85 (K14245), ab56+c86 (K14246), ab56+c87 (K14247), ab56+c88 (K14248), ab56+c89 (K14249), ab56+c90 (K14250), ab56+c91 (K14251), ab56+c92 (K14252), ab56+c93 (K14253), ab56+c94 (K14254), ab56+c95 (K14255), ab56+c96 (K14256), ab56+c97 (K14257), ab56+c98 (K14258), ab56+c99 (K14259), ab56+c100 (K14260), ab56+c101 (K14261), ab56+c102 (K14262), ab56+c103 (K14263), ab56+c104 (K14264), ab56+c105 (K14265), ab56+c106 (K14266), ab56+c107 (K14267), ab56+c108 (K14268), ab56+c109 (K14269), ab56+c110 (K14270), ab56+c111 (K14271), ab56+c112 (K14272), ab56+c113 (K14273), ab56+c114 (K14274), ab56+c115 (K14275), ab56+c116 (K14276), ab56+c117 (K14277), ab56+c118 (K14278), ab56+c119 (K14279), ab56+c120 (K14280), ab56+c121 (K14281), ab56+c122 (K14282), ab56+c123 (K14283), ab56+c124 (K14284), ab56+c125 (K14285), ab56+c126 (K14286), ab56+c127 (K14287), ab56+c128 (K14288), ab56+c129 (K14289), ab56+c130 (K14290), ab56+c131 (K14291), ab56+c132 (K14292), ab56+c133 (K14293), ab56+c134 (K14294), ab56+c135 (K14295), ab56+c136 (K14296), ab56+c137 (K14297), ab56+c138 (K14298), ab56+c139 (K14299), ab56+c140 (K14300), ab56+c141 (K14301), ab56+c142 (K14302), ab56+c143 (K14303), ab56+c144 (K14304), ab56+c145 (K14305), ab56+c146 (K14306), ab56+c147 (K14307), ab56+c148 (K14308), ab56+c149 (K14309), ab56+c150 (K14310), ab56+c151 (K14311), ab56+c152 (K14312), ab56+c153 (K14313), ab56+c154 (K14314), ab56+c155 (K14315), ab56+c156 (K14316), ab56+c157 (K14317), ab56+c158 (K14318), ab56+c159 (K14319), ab56+c160 (K14320), ab56+c161 (K14321), ab56+c162 (K14322), ab56+c163 (K14323), ab56+c164 (K14324), ab56+c165 (K14325), ab56+c166 (K14326), ab56+c167 (K14327), ab56+c168 (K14328), ab56+c169 (K14329), ab56+c170 (K14330), ab56+c171 (K14331), ab56+c172 (K14332), ab56+c173 (K14333), ab56+c174 (K14334), ab56+c175 (K14335), ab56+c176 (K14336), ab56+c177 (K14337), ab56+c178 (K14338), ab56+c179 (K14339), ab56+c180 (K14340), ab56+c181 (K14341), ab56+c182 (K14342), ab56+c183 (K14343), ab56+c184 (K14344), ab56+c185 (K14345), ab56+c186 (K14346), ab56+c187 (K14347), ab56+c188 (K14348), ab56+c189 (K14349), ab56+c190 (K14350), ab56+c191 (K14351), ab56+c192 (K14352), ab56+c193 (K14353), ab56+c194 (K14354), ab56+c195 (K14355), ab56+c196 (K14356), ab56+c197 (K14357), ab56+c198 (K14358), ab56+c199 (K14359), ab56+c200 (K14360), ab57+c1 (K14361), ab57+c2 (K14362), ab57+c3 (K14363), ab57+c4 (K14364), ab57+c5 (K14365), ab57+c6 (K14366), ab57+c7 (K14367), ab57+c8 (K14368), ab57+c9 (K14369), ab57+c10 (K14370), ab57+c11 (K14371), ab57+c12 (K14372), ab57+c13 (K14373), ab57+c14 (K14374), ab57+c15 (K14375), ab57+c16 (K14376), ab57+c17 (K14377), ab57+c18 (K14378), ab57+c19 (K14379), ab57+c20 (K14380), ab57+c21 (K14381), ab57+c22 (K14382), ab57+c23 (K14383), ab57+c24 (K14384), ab57+c25 (K14385), ab57+c26 (K14386), ab57+c27 (K14387), ab57+c28 (K14388), ab57+c29 (K14389), ab57+c30 (K14390), ab57+c31 (K14391), ab57+c32 (K14392), ab57+c33 (K14393), ab57+c34 (K14394), ab57+c35 (K14395), ab57+c36 (K14396), ab57+c37 (K14397), ab57+c38 (K14398), ab57+c39 (K14399), ab57+c40 (K14400), ab57+c41 (K14401), ab57+c42 (K14402), ab57+c43 (K14403), ab57+c44 (K14404), ab57+c45 (K14405), ab57+c46 (K14406), ab57+c47 (K14407), ab57+c48 (K14408), ab57+c49 (K14409), ab57+c50 (K14410), ab57+c51 (K14411), ab57+c52 (K14412), ab57+c53 (K14413), ab57+c54 (K14414), ab57+c55 (K14415), ab57+c56 (K14416), ab57+c57 (K14417), ab57+c58 (K14418), ab57+c59 (K14419), ab57+c60 (K14420), ab57+c61 (K14421), ab57+c62 (K14422), ab57+c63 (K14423), ab57+c64 (K14424), ab57+c65 (K14425), ab57+c66 (K14426), ab57+c67 (K14427), ab57+c68 (K14428), ab57+c69 (K14429), ab57+c70 (K14430), ab57+c71 (K14431), ab57+c72 (K14432), ab57+c73 (K14433), ab57+c74 (K14434), ab57+c75 (K14435), ab57+c76 (K14436), ab57+c77 (K14437), ab57+c78 (K14438), ab57+c79 (K14439), ab57+c80 (K14440), ab57+c81 (K14441), ab57+c82 (K14442), ab57+c83 (K14443), ab57+c84 (K14444), ab57+c85 (K14445), ab57+c86 (K14446), ab57+c87 (K14447), ab57+c88 (K14448), ab57+c89 (K14449), ab57+c90 (K14450), ab57+c91 (K14451), ab57+c92 (K14452), ab57+c93 (K14453), ab57+c94 (K14454), ab57+c95 (K14455), ab57+c96 (K14456), ab57+c97 (K14457), ab57+c98 (K14458), ab57+c99 (K14459), ab57+c100 (K14460), ab57+c101 (K14461), ab57+c102 (K14462), ab57+c103 (K14463), ab57+c104 (K14464), ab57+c105 (K14465), ab57+c106 (K14466), ab57+c107 (K14467), ab57+c108 (K14468), ab57+c109 (K14469), ab57+c110 (K14470), ab57+c111 (K14471), ab57+c112 (K14472), ab57+c113 (K14473), ab57+c114 (K14474), ab57+c115 (K14475), ab57+c116 (K14476), ab57+c117 (K14477), ab57+c118 (K14478), ab57+c119 (K14479), ab57+c120 (K14480), ab57+c121 (K14481), ab57+c122 (K14482), ab57+c123 (K14483), ab57+c124 (K14484), ab57+c125 (K14485), ab57+c126 (K14486), ab57+c127 (K14487), ab57+c128 (K14488), ab57+c129 (K14489), ab57+c130 (K14490), ab57+c131 (K14491), ab57+c132 (K14492), ab57+c133 (K14493), ab57+c134 (K14494), ab57+c135 (K14495), ab57+c136 (K14496), ab57+c137 (K14497), ab57+c138 (K14498), ab57+c139 (K14499), ab57+c140 (K14500), ab57+c141 (K14501), ab57+c142 (K14502), ab57+c143 (K14503), ab57+c144 (K14504), ab57+c145 (K14505), ab57+c146 (K14506), ab57+c147 (K14507), ab57+c148 (K14508), ab57+c149 (K14509), ab57+c150 (K14510), ab57+c151 (K14511), ab57+c152 (K14512), ab57+c153 (K14513), ab57+c154 (K14514), ab57+c155 (K14515), ab57+c156 (K14516), ab57+c157 (K14517), ab57+c158 (K14518), ab57+c159 (K14519), ab57+c160 (K14520), ab57+c161 (K14521), ab57+c162 (K14522), ab57+c163 (K14523), ab57+c164 (K14524), ab57+c165 (K14525), ab57+c166 (K14526), ab57+c167 (K14527), ab57+c168 (K14528), ab57+c169 (K14529), ab57+c170 (K14530), ab57+c171 (K14531), ab57+c172 (K14532), ab57+c173 (K14533), ab57+c174 (K14534), ab57+c175 (K14535), ab57+c176 (K14536), ab57+c177 (K14537), ab57+c178 (K14538), ab57+c179 (K14539), ab57+c180 (K14540), ab57+c181 (K14541), ab57+c182 (K14542), ab57+c183 (K14543), ab57+c184 (K14544), ab57+c185 (K14545), ab57+c186 (K14546), ab57+c187 (K14547), ab57+c188 (K14548), ab57+c189 (K14549), ab57+c190 (K14550), ab57+c191 (K14551), ab57+c192 (K14552), ab57+c193 (K14553), ab57+c194 (K14554), ab57+c195 (K14555), ab57+c196 (K14556), ab57+c197 (K14557), ab57+c198 (K14558), ab57+c199 (K14559), ab57+c200 (K14560), ab58+c1 (K14561), ab58+c2 (K14562), ab58+c3 (K14563), ab58+c4 (K14564), ab58+c5 (K14565), ab58+c6 (K14566), ab58+c7 (K14567), ab58+c8 (K14568), ab58+c9 (K14569), ab58+c10 (K14570), ab58+c11 (K14571), ab58+c12 (K14572), ab58+c13 (K14573), ab58+c14 (K14574), ab58+c15 (K14575), ab58+c16 (K14576), ab58+c17 (K14577), ab58+c18 (K14578), ab58+c19 (K14579), ab58+c20 (K14580), ab58+c21 (K14581), ab58+c22 (K14582), ab58+c23 (K14583), ab58+c24 (K14584), ab58+c25 (K14585), ab58+c26 (K14586), ab58+c27 (K14587), ab58+c28 (K14588), ab58+c29 (K14589), ab58+c30 (K14590), ab58+c31 (K14591), ab58+c32 (K14592), ab58+c33 (K14593), ab58+c34 (K14594), ab58+c35 (K14595), ab58+c36 (K14596), ab58+c37 (K14597), ab58+c38 (K14598), ab58+c39 (K14599), ab58+c40 (K14600), ab58+c41 (K14601), ab58+c42 (K14602), ab58+c43 (K14603), ab58+c44 (K14604), ab58+c45 (K14605), ab58+c46 (K14606), ab58+c47 (K14607), ab58+c48 (K14608), ab58+c49 (K14609), ab58+c50 (K14610), ab58+c51 (K14611), ab58+c52 (K14612), ab58+c53 (K14613), ab58+c54 (K14614), ab58+c55 (K14615), ab58+c56 (K14616), ab58+c57 (K14617), ab58+c58 (K14618), ab58+c59 (K14619), ab58+c60 (K14620), ab58+c61 (K14621), ab58+c62 (K14622), ab58+c63 (K14623), ab58+c64 (K14624), ab58+c65 (K14625), ab58+c66 (K14626), ab58+c67 (K14627), ab58+c68 (K14628), ab58+c69 (K14629), ab58+c70 (K14630), ab58+c71 (K14631), ab58+c72 (K14632), ab58+c73 (K14633), ab58+c74 (K14634), ab58+c75 (K14635), ab58+c76 (K14636), ab58+c77 (K14637), ab58+c78 (K14638), ab58+c79 (K14639), ab58+c80 (K14640), ab58+c81 (K14641), ab58+c82 (K14642), ab58+c83 (K14643), ab58+c84 (K14644), ab58+c85 (K14645), ab58+c86 (K14646), ab58+c87 (K14647), ab58+c88 (K14648), ab58+c89 (K14649), ab58+c90 (K14650), ab58+c91 (K14651), ab58+c92 (K14652), ab58+c93 (K14653), ab58+c94 (K14654), ab58+c95 (K14655), ab58+c96 (K14656), ab58+c97 (K14657), ab58+c98 (K14658), ab58+c99 (K14659), ab58+c100 (K14660), ab58+c101 (K14661), ab58+c102 (K14662), ab58+c103 (K14663), ab58+c104 (K14664), ab58+c105 (K14665), ab58+c106 (K14666), ab58+c107 (K14667), ab58+c108 (K14668), ab58+c109 (K14669), ab58+c110 (K14670), ab58+c111 (K14671), ab58+c112 (K14672), ab58+c113 (K14673), ab58+c114 (K14674), ab58+c115 (K14675), ab58+c116 (K14676), ab58+c117 (K14677), ab58+c118 (K14678), ab58+c119 (K14679), ab58+c120 (K14680), ab58+c121 (K14681), ab58+c122 (K14682), ab58+c123 (K14683), ab58+c124 (K14684), ab58+c125 (K14685), ab58+c126 (K14686), ab58+c127 (K14687), ab58+c128 (K14688), ab58+c129 (K14689), ab58+c130 (K14690), ab58+c131 (K14691), ab58+c132 (K14692), ab58+c133 (K14693), ab58+c134 (K14694), ab58+c135 (K14695), ab58+c136 (K14696), ab58+c137 (K14697), ab58+c138 (K14698), ab58+c139 (K14699), ab58+c140 (K14700), ab58+c141 (K14701), ab58+c142 (K14702), ab58+c143 (K14703), ab58+c144 (K14704), ab58+c145 (K14705), ab58+c146 (K14706), ab58+c147 (K14707), ab58+c148 (K14708), ab58+c149 (K14709), ab58+c150 (K14710), ab58+c151 (K14711), ab58+c152 (K14712), ab58+c153 (K14713), ab58+c154 (K14714), ab58+c155 (K14715), ab58+c156 (K14716), ab58+c157 (K14717), ab58+c158 (K14718), ab58+c159 (K14719), ab58+c160 (K14720), ab58+c161 (K14721), ab58+c162 (K14722), ab58+c163 (K14723), ab58+c164 (K14724), ab58+c165 (K14725), ab58+c166 (K14726), ab58+c167 (K14727), ab58+c168 (K14728), ab58+c169 (K14729), ab58+c170 (K14730), ab58+c171 (K14731), ab58+c172 (K14732), ab58+c173 (K14733), ab58+c174 (K14734), ab58+c175 (K14735), ab58+c176 (K14736), ab58+c177 (K14737), ab58+c178 (K14738), ab58+c179 (K14739), ab58+c180 (K14740), ab58+c181 (K14741), ab58+c182 (K14742), ab58+c183 (K14743), ab58+c184 (K14744), ab58+c185 (K14745), ab58+c186 (K14746), ab58+c187 (K14747), ab58+c188 (K14748), ab58+c189 (K14749), ab58+c190 (K14750), ab58+c191 (K14751), ab58+c192 (K14752), ab58+c193 (K14753), ab58+c194 (K14754), ab58+c195 (K14755), ab58+c196 (K14756), ab58+c197 (K14757), ab58+c198 (K14758), ab58+c199 (K14759), ab58+c200 (K14760), ab59+c1 (K14761), ab59+c2 (K14762), ab59+c3 (K14763), ab59+c4 (K14764), ab59+c5 (K14765), ab59+c6 (K14766), ab59+c7 (K14767), ab59+c8 (K14768), ab59+c9 (K14769), ab59+c10 (K14770), ab59+c11 (K14771), ab59+c12 (K14772), ab59+c13 (K14773), ab59+c14 (K14774), ab59+c15 (K14775), ab59+c16 (K14776), ab59+c17 (K14777), ab59+c18 (K14778), ab59+c19 (K14779), ab59+c20 (K14780), ab59+c21 (K14781), ab59+c22 (K14782), ab59+c23 (K14783), ab59+c24 (K14784), ab59+c25 (K14785), ab59+c26 (K14786), ab59+c27 (K14787), ab59+c28 (K14788), ab59+c29 (K14789), ab59+c30 (K14790), ab59+c31 (K14791), ab59+c32 (K14792), ab59+c33 (K14793), ab59+c34 (K14794), ab59+c35 (K14795), ab59+c36 (K14796), ab59+c37 (K14797), ab59+c38 (K14798), ab59+c39 (K14799), ab59+c40 (K14800), ab59+c41 (K14801), ab59+c42 (K14802), ab59+c43 (K14803), ab59+c44 (K14804), ab59+c45 (K14805), ab59+c46 (K14806), ab59+c47 (K14807), ab59+c48 (K14808), ab59+c49 (K14809), ab59+c50 (K14810), ab59+c51 (K14811), ab59+c52 (K14812), ab59+c53 (K14813), ab59+c54 (K14814), ab59+c55 (K14815), ab59+c56 (K14816), ab59+c57 (K14817), ab59+c58 (K14818), ab59+c59 (K14819), ab59+c60 (K14820), ab59+c61 (K14821), ab59+c62 (K14822), ab59+c63 (K14823), ab59+c64 (K14824), ab59+c65 (K14825), ab59+c66 (K14826), ab59+c67 (K14827), ab59+c68 (K14828), ab59+c69 (K14829), ab59+c70 (K14830), ab59+c71 (K14831), ab59+c72 (K14832), ab59+c73 (K14833), ab59+c74 (K14834), ab59+c75 (K14835), ab59+c76 (K14836), ab59+c77 (K14837), ab59+c78 (K14838), ab59+c79 (K14839), ab59+c80 (K14840), ab59+c81 (K14841), ab59+c82 (K14842), ab59+c83 (K14843), ab59+c84 (K14844), ab59+c85 (K14845), ab59+c86 (K14846), ab59+c87 (K14847), ab59+c88 (K14848), ab59+c89 (K14849), ab59+c90 (K14850), ab59+c91 (K14851), ab59+c92 (K14852), ab59+c93 (K14853), ab59+c94 (K14854), ab59+c95 (K14855), ab59+c96 (K14856), ab59+c97 (K14857), ab59+c98 (K14858), ab59+c99 (K14859), ab59+c100 (K14860), ab59+c101 (K14861), ab59+c102 (K14862), ab59+c103 (K14863), ab59+c104 (K14864), ab59+c105 (K14865), ab59+c106 (K14866), ab59+c107 (K14867), ab59+c108 (K14868), ab59+c109 (K14869), ab59+c110 (K14870), ab59+c111 (K14871), ab59+c112 (K14872), ab59+c113 (K14873), ab59+c114 (K14874), ab59+c115 (K14875), ab59+c116 (K14876), ab59+c117 (K14877), ab59+c118 (K14878), ab59+c119 (K14879), ab59+c120 (K14880), ab59+c121 (K14881), ab59+c122 (K14882), ab59+c123 (K14883), ab59+c124 (K14884), ab59+c125 (K14885), ab59+c126 (K14886), ab59+c127 (K14887), ab59+c128 (K14888), ab59+c129 (K14889), ab59+c130 (K14890), ab59+c131 (K14891), ab59+c132 (K14892), ab59+c133 (K14893), ab59+c134 (K14894), ab59+c135 (K14895), ab59+c136 (K14896), ab59+c137 (K14897), ab59+c138 (K14898), ab59+c139 (K14899), ab59+c140 (K14900), ab59+c141 (K14901), ab59+c142 (K14902), ab59+c143 (K14903), ab59+c144 (K14904), ab59+c145 (K14905), ab59+c146 (K14906), ab59+c147 (K14907), ab59+c148 (K14908), ab59+c149 (K14909), ab59+c150 (K14910), ab59+c151 (K14911), ab59+c152 (K14912), ab59+c153 (K14913), ab59+c154 (K14914), ab59+c155 (K14915), ab59+c156 (K14916), ab59+c157 (K14917), ab59+c158 (K14918), ab59+c159 (K14919), ab59+c160 (K14920), ab59+c161 (K14921), ab59+c162 (K14922), ab59+c163 (K14923), ab59+c164 (K14924), ab59+c165 (K14925), ab59+c166 (K14926), ab59+c167 (K14927), ab59+c168 (K14928), ab59+c169 (K14929), ab59+c170 (K14930), ab59+c171 (K14931), ab59+c172 (K14932), ab59+c173 (K14933), ab59+c174 (K14934), ab59+c175 (K14935), ab59+c176 (K14936), ab59+c177 (K14937), ab59+c178 (K14938), ab59+c179 (K14939), ab59+c180 (K14940), ab59+c181 (K14941), ab59+c182 (K14942), ab59+c183 (K14943), ab59+c184 (K14944), ab59+c185 (K14945), ab59+c186 (K14946), ab59+c187 (K14947), ab59+c188 (K14948), ab59+c189 (K14949), ab59+c190 (K14950), ab59+c191 (K14951), ab59+c192 (K14952), ab59+c193 (K14953), ab59+c194 (K14954), ab59+c195 (K14955), ab59+c196 (K14956), ab59+c197 (K14957), ab59+c198 (K14958), ab59+c199 (K14959), ab59+c200 (K14960), ab60+c1 (K14961), ab60+c2 (K14962), ab60+c3 (K14963), ab60+c4 (K14964), ab60+c5 (K14965), ab60+c6 (K14966), ab60+c7 (K14967), ab60+c8 (K14968), ab60+c9 (K14969), ab60+c10 (K14970), ab60+c11 (K14971), ab60+c12 (K14972), ab60+c13 (K14973), ab60+c14 (K14974), ab60+c15 (K14975), ab60+c16 (K14976), ab60+c17 (K14977), ab60+c18 (K14978), ab60+c19 (K14979), ab60+c20 (K14980), ab60+c21 (K14981), ab60+c22 (K14982), ab60+c23 (K14983), ab60+c24 (K14984), ab60+c25 (K14985), ab60+c26 (K14986), ab60+c27 (K14987), ab60+c28 (K14988), ab60+c29 (K14989), ab60+c30 (K14990), ab60+c31 (K14991), ab60+c32 (K14992), ab60+c33 (K14993), ab60+c34 (K14994), ab60+c35 (K14995), ab60+c36 (K14996), ab60+c37 (K14997), ab60+c38 (K14998), ab60+c39 (K14999), ab60+c40 (K15000), ab60+c41 (K15001), ab60+c42 (K15002), ab60+c43 (K15003), ab60+c44 (K15004), ab60+c45 (K15005), ab60+c46 (K15006), ab60+c47 (K15007), ab60+c48 (K15008), ab60+c49 (K15009), ab60+c50 (K15010), ab60+c51 (K15011), ab60+c52 (K15012), ab60+c53 (K15013), ab60+c54 (K15014), ab60+c55 (K15015), ab60+c56 (K15016), ab60+c57 (K15017), ab60+c58 (K15018), ab60+c59 (K15019), ab60+c60 (K15020), ab60+c61 (K15021), ab60+c62 (K15022), ab60+c63 (K15023), ab60+c64 (K15024), ab60+c65 (K15025), ab60+c66 (K15026), ab60+c67 (K15027), ab60+c68 (K15028), ab60+c69 (K15029), ab60+c70 (K15030), ab60+c71 (K15031), ab60+c72 (K15032), ab60+c73 (K15033), ab60+c74 (K15034), ab60+c75 (K15035), ab60+c76 (K15036), ab60+c77 (K15037), ab60+c78 (K15038), ab60+c79 (K15039), ab60+c80 (K15040), ab60+c81 (K15041), ab60+c82 (K15042), ab60+c83 (K15043), ab60+c84 (K15044), ab60+c85 (K15045), ab60+c86 (K15046), ab60+c87 (K15047), ab60+c88 (K15048), ab60+c89 (K15049), ab60+c90 (K15050), ab60+c91 (K15051), ab60+c92 (K15052), ab60+c93 (K15053), ab60+c94 (K15054), ab60+c95 (K15055), ab60+c96 (K15056), ab60+c97 (K15057), ab60+c98 (K15058), ab60+c99 (K15059), ab60+c100 (K15060), ab60+c101 (K15061), ab60+c102 (K15062), ab60+c103 (K15063), ab60+c104 (K15064), ab60+c105 (K15065), ab60+c106 (K15066), ab60+c107 (K15067), ab60+c108 (K15068), ab60+c109 (K15069), ab60+c110 (K15070), ab60+c111 (K15071), ab60+c112 (K15072), ab60+c113 (K15073), ab60+c114 (K15074), ab60+c115 (K15075), ab60+c116 (K15076), ab60+c117 (K15077), ab60+c118 (K15078), ab60+c119 (K15079), ab60+c120 (K15080), ab60+c121 (K15081), ab60+c122 (K15082), ab60+c123 (K15083), ab60+c124 (K15084), ab60+c125 (K15085), ab60+c126 (K15086), ab60+c127 (K15087), ab60+c128 (K15088), ab60+c129 (K15089), ab60+c130 (K15090), ab60+c131 (K15091), ab60+c132 (K15092), ab60+c133 (K15093), ab60+c134 (K15094), ab60+c135 (K15095), ab60+c136 (K15096), ab60+c137 (K15097), ab60+c138 (K15098), ab60+c139 (K15099), ab60+c140 (K15100), ab60+c141 (K15101), ab60+c142 (K15102), ab60+c143 (K15103), ab60+c144 (K15104), ab60+c145 (K15105), ab60+c146 (K15106), ab60+c147 (K15107), ab60+c148 (K15108), ab60+c149 (K15109), ab60+c150 (K15110), ab60+c151 (K15111), ab60+c152 (K15112), ab60+c153 (K15113), ab60+c154 (K15114), ab60+c155 (K15115), ab60+c156 (K15116), ab60+c157 (K15117), ab60+c158 (K15118), ab60+c159 (K15119), ab60+c160 (K15120), ab60+c161 (K15121), ab60+c162 (K15122), ab60+c163 (K15123), ab60+c164 (K15124), ab60+c165 (K15125), ab60+c166 (K15126), ab60+c167 (K15127), ab60+c168 (K15128), ab60+c169 (K15129), ab60+c170 (K15130), ab60+c171 (K15131), ab60+c172 (K15132), ab60+c173 (K15133), ab60+c174 (K15134), ab60+c175 (K15135), ab60+c176 (K15136), ab60+c177 (K15137), ab60+c178 (K15138), ab60+c179 (K15139), ab60+c180 (K15140), ab60+c181 (K15141), ab60+c182 (K15142), ab60+c183 (K15143), ab60+c184 (K15144), ab60+c185 (K15145), ab60+c186 (K15146), ab60+c187 (K15147), ab60+c188 (K15148), ab60+c189 (K15149), ab60+c190 (K15150), ab60+c191 (K15151), ab60+c192 (K15152), ab60+c193 (K15153), ab60+c194 (K15154), ab60+c195 (K15155), ab60+c196 (K15156), ab60+c197 (K15157), ab60+c198 (K15158), ab60+c199 (K15159), ab60+c200 (K15160), ab61+c1 (K15161), ab61+c2 (K15162), ab61+c3 (K15163), ab61+c4 (K15164), ab61+c5 (K15165), ab61+c6 (K15166), ab61+c7 (K15167), ab61+c8 (K15168), ab61+c9 (K15169), ab61+c10 (K15170), ab61+c11 (K15171), ab61+c12 (K15172), ab61+c13 (K15173), ab61+c14 (K15174), ab61+c15 (K15175), ab61+c16 (K15176), ab61+c17 (K15177), ab61+c18 (K15178), ab61+c19 (K15179), ab61+c20 (K15180), ab61+c21 (K15181), ab61+c22 (K15182), ab61+c23 (K15183), ab61+c24 (K15184), ab61+c25 (K15185), ab61+c26 (K15186), ab61+c27 (K15187), ab61+c28 (K15188), ab61+c29 (K15189), ab61+c30 (K15190), ab61+c31 (K15191), ab61+c32 (K15192), ab61+c33 (K15193), ab61+c34 (K15194), ab61+c35 (K15195), ab61+c36 (K15196), ab61+c37 (K15197), ab61+c38 (K15198), ab61+c39 (K15199), ab61+c40 (K15200), ab61+c41 (K15201), ab61+c42 (K15202), ab61+c43 (K15203), ab61+c44 (K15204), ab61+c45 (K15205), ab61+c46 (K15206), ab61+c47 (K15207), ab61+c48 (K15208), ab61+c49 (K15209), ab61+c50 (K15210), ab61+c51 (K15211), ab61+c52 (K15212), ab61+c53 (K15213), ab61+c54 (K15214), ab61+c55 (K15215), ab61+c56 (K15216), ab61+c57 (K15217), ab61+c58 (K15218), ab61+c59 (K15219), ab61+c60 (K15220), ab61+c61 (K15221), ab61+c62 (K15222), ab61+c63 (K15223), ab61+c64 (K15224), ab61+c65 (K15225), ab61+c66 (K15226), ab61+c67 (K15227), ab61+c68 (K15228), ab61+c69 (K15229), ab61+c70 (K15230), ab61+c71 (K15231), ab61+c72 (K15232), ab61+c73 (K15233), ab61+c74 (K15234), ab61+c75 (K15235), ab61+c76 (K15236), ab61+c77 (K15237), ab61+c78 (K15238), ab61+c79 (K15239), ab61+c80 (K15240), ab61+c81 (K15241), ab61+c82 (K15242), ab61+c83 (K15243), ab61+c84 (K15244), ab61+c85 (K15245), ab61+c86 (K15246), ab61+c87 (K15247), ab61+c88 (K15248), ab61+c89 (K15249), ab61+c90 (K15250), ab61+c91 (K15251), ab61+c92 (K15252), ab61+c93 (K15253), ab61+c94 (K15254), ab61+c95 (K15255), ab61+c96 (K15256), ab61+c97 (K15257), ab61+c98 (K15258), ab61+c99 (K15259), ab61+c100 (K15260), ab61+c101 (K15261), ab61+c102 (K15262), ab61+c103 (K15263), ab61+c104 (K15264), ab61+c105 (K15265), ab61+c106 (K15266), ab61+c107 (K15267), ab61+c108 (K15268), ab61+c109 (K15269), ab61+c110 (K15270), ab61+c111 (K15271), ab61+c112 (K15272), ab61+c113 (K15273), ab61+c114 (K15274), ab61+c115 (K15275), ab61+c116 (K15276), ab61+c117 (K15277), ab61+c118 (K15278), ab61+c119 (K15279), ab61+c120 (K15280), ab61+c121 (K15281), ab61+c122 (K15282), ab61+c123 (K15283), ab61+c124 (K15284), ab61+c125 (K15285), ab61+c126 (K15286), ab61+c127 (K15287), ab61+c128 (K15288), ab61+c129 (K15289), ab61+c130 (K15290), ab61+c131 (K15291), ab61+c132 (K15292), ab61+c133 (K15293), ab61+c134 (K15294), ab61+c135 (K15295), ab61+c136 (K15296), ab61+c137 (K15297), ab61+c138 (K15298), ab61+c139 (K15299), ab61+c140 (K15300), ab61+c141 (K15301), ab61+c142 (K15302), ab61+c143 (K15303), ab61+c144 (K15304), ab61+c145 (K15305), ab61+c146 (K15306), ab61+c147 (K15307), ab61+c148 (K15308), ab61+c149 (K15309), ab61+c150 (K15310), ab61+c151 (K15311), ab61+c152 (K15312), ab61+c153 (K15313), ab61+c154 (K15314), ab61+c155 (K15315), ab61+c156 (K15316), ab61+c157 (K15317), ab61+c158 (K15318), ab61+c159 (K15319), ab61+c160 (K15320), ab61+c161 (K15321), ab61+c162 (K15322), ab61+c163 (K15323), ab61+c164 (K15324), ab61+c165 (K15325), ab61+c166 (K15326), ab61+c167 (K15327), ab61+c168 (K15328), ab61+c169 (K15329), ab61+c170 (K15330), ab61+c171 (K15331), ab61+c172 (K15332), ab61+c173 (K15333), ab61+c174 (K15334), ab61+c175 (K15335), ab61+c176 (K15336), ab61+c177 (K15337), ab61+c178 (K15338), ab61+c179 (K15339), ab61+c180 (K15340), ab61+c181 (K15341), ab61+c182 (K15342), ab61+c183 (K15343), ab61+c184 (K15344), ab61+c185 (K15345), ab61+c186 (K15346), ab61+c187 (K15347), ab61+c188 (K15348), ab61+c189 (K15349), ab61+c190 (K15350), ab61+c191 (K15351), ab61+c192 (K15352), ab61+c193 (K15353), ab61+c194 (K15354), ab61+c195 (K15355), ab61+c196 (K15356), ab61+c197 (K15357), ab61+c198 (K15358), ab61+c199 (K15359), ab61+c200 (K15360), ab62+c1 (K15361), ab62+c2 (K15362), ab62+c3 (K15363), ab62+c4 (K15364), ab62+c5 (K15365), ab62+c6 (K15366), ab62+c7 (K15367), ab62+c8 (K15368), ab62+c9 (K15369), ab62+c10 (K15370), ab62+c11 (K15371), ab62+c12 (K15372), ab62+c13 (K15373), ab62+c14 (K15374), ab62+c15 (K15375), ab62+c16 (K15376), ab62+c17 (K15377), ab62+c18 (K15378), ab62+c19 (K15379), ab62+c20 (K15380), ab62+c21 (K15381), ab62+c22 (K15382), ab62+c23 (K15383), ab62+c24 (K15384), ab62+c25 (K15385), ab62+c26 (K15386), ab62+c27 (K15387), ab62+c28 (K15388), ab62+c29 (K15389), ab62+c30 (K15390), ab62+c31 (K15391), ab62+c32 (K15392), ab62+c33 (K15393), ab62+c34 (K15394), ab62+c35 (K15395), ab62+c36 (K15396), ab62+c37 (K15397), ab62+c38 (K15398), ab62+c39 (K15399), ab62+c40 (K15400), ab62+c41 (K15401), ab62+c42 (K15402), ab62+c43 (K15403), ab62+c44 (K15404), ab62+c45 (K15405), ab62+c46 (K15406), ab62+c47 (K15407), ab62+c48 (K15408), ab62+c49 (K15409), ab62+c50 (K15410), ab62+c51 (K15411), ab62+c52 (K15412), ab62+c53 (K15413), ab62+c54 (K15414), ab62+c55 (K15415), ab62+c56 (K15416), ab62+c57 (K15417), ab62+c58 (K15418), ab62+c59 (K15419), ab62+c60 (K15420), ab62+c61 (K15421), ab62+c62 (K15422), ab62+c63 (K15423), ab62+c64 (K15424), ab62+c65 (K15425), ab62+c66 (K15426), ab62+c67 (K15427), ab62+c68 (K15428), ab62+c69 (K15429), ab62+c70 (K15430), ab62+c71 (K15431), ab62+c72 (K15432), ab62+c73 (K15433), ab62+c74 (K15434), ab62+c75 (K15435), ab62+c76 (K15436), ab62+c77 (K15437), ab62+c78 (K15438), ab62+c79 (K15439), ab62+c80 (K15440), ab62+c81 (K15441), ab62+c82 (K15442), ab62+c83 (K15443), ab62+c84 (K15444), ab62+c85 (K15445), ab62+c86 (K15446), ab62+c87 (K15447), ab62+c88 (K15448), ab62+c89 (K15449), ab62+c90 (K15450), ab62+c91 (K15451), ab62+c92 (K15452), ab62+c93 (K15453), ab62+c94 (K15454), ab62+c95 (K15455), ab62+c96 (K15456), ab62+c97 (K15457), ab62+c98 (K15458), ab62+c99 (K15459), ab62+c100 (K15460), ab62+c101 (K15461), ab62+c102 (K15462), ab62+c103 (K15463), ab62+c104 (K15464), ab62+c105 (K15465), ab62+c106 (K15466), ab62+c107 (K15467), ab62+c108 (K15468), ab62+c109 (K15469), ab62+c110 (K15470), ab62+c111 (K15471), ab62+c112 (K15472), ab62+c113 (K15473), ab62+c114 (K15474), ab62+c115 (K15475), ab62+c116 (K15476), ab62+c117 (K15477), ab62+c118 (K15478), ab62+c119 (K15479), ab62+c120 (K15480), ab62+c121 (K15481), ab62+c122 (K15482), ab62+c123 (K15483), ab62+c124 (K15484), ab62+c125 (K15485), ab62+c126 (K15486), ab62+c127 (K15487), ab62+c128 (K15488), ab62+c129 (K15489), ab62+c130 (K15490), ab62+c131 (K15491), ab62+c132 (K15492), ab62+c133 (K15493), ab62+c134 (K15494), ab62+c135 (K15495), ab62+c136 (K15496), ab62+c137 (K15497), ab62+c138 (K15498), ab62+c139 (K15499), ab62+c140 (K15500), ab62+c141 (K15501), ab62+c142 (K15502), ab62+c143 (K15503), ab62+c144 (K15504), ab62+c145 (K15505), ab62+c146 (K15506), ab62+c147 (K15507), ab62+c148 (K15508), ab62+c149 (K15509), ab62+c150 (K15510), ab62+c151 (K15511), ab62+c152 (K15512), ab62+c153 (K15513), ab62+c154 (K15514), ab62+c155 (K15515), ab62+c156 (K15516), ab62+c157 (K15517), ab62+c158 (K15518), ab62+c159 (K15519), ab62+c160 (K15520), ab62+c161 (K15521), ab62+c162 (K15522), ab62+c163 (K15523), ab62+c164 (K15524), ab62+c165 (K15525), ab62+c166 (K15526), ab62+c167 (K15527), ab62+c168 (K15528), ab62+c169 (K15529), ab62+c170 (K15530), ab62+c171 (K15531), ab62+c172 (K15532), ab62+c173 (K15533), ab62+c174 (K15534), ab62+c175 (K15535), ab62+c176 (K15536), ab62+c177 (K15537), ab62+c178 (K15538), ab62+c179 (K15539), ab62+c180 (K15540), ab62+c181 (K15541), ab62+c182 (K15542), ab62+c183 (K15543), ab62+c184 (K15544), ab62+c185 (K15545), ab62+c186 (K15546), ab62+c187 (K15547), ab62+c188 (K15548), ab62+c189 (K15549), ab62+c190 (K15550), ab62+c191 (K15551), ab62+c192 (K15552), ab62+c193 (K15553), ab62+c194 (K15554), ab62+c195 (K15555), ab62+c196 (K15556), ab62+c197 (K15557), ab62+c198 (K15558), ab62+c199 (K15559), ab62+c200 (K15560), ab63+c1 (K15561), ab63+c2 (K15562), ab63+c3 (K15563), ab63+c4 (K15564), ab63+c5 (K15565), ab63+c6 (K15566), ab63+c7 (K15567), ab63+c8 (K15568), ab63+c9 (K15569), ab63+c10 (K15570), ab63+c11 (K15571), ab63+c12 (K15572), ab63+c13 (K15573), ab63+c14 (K15574), ab63+c15 (K15575), ab63+c16 (K15576), ab63+c17 (K15577), ab63+c18 (K15578), ab63+c19 (K15579), ab63+c20 (K15580), ab63+c21 (K15581), ab63+c22 (K15582), ab63+c23 (K15583), ab63+c24 (K15584), ab63+c25 (K15585), ab63+c26 (K15586), ab63+c27 (K15587), ab63+c28 (K15588), ab63+c29 (K15589), ab63+c30 (K15590), ab63+c31 (K15591), ab63+c32 (K15592), ab63+c33 (K15593), ab63+c34 (K15594), ab63+c35 (K15595), ab63+c36 (K15596), ab63+c37 (K15597), ab63+c38 (K15598), ab63+c39 (K15599), ab63+c40 (K15600), ab63+c41 (K15601), ab63+c42 (K15602), ab63+c43 (K15603), ab63+c44 (K15604), ab63+c45 (K15605), ab63+c46 (K15606), ab63+c47 (K15607), ab63+c48 (K15608), ab63+c49 (K15609), ab63+c50 (K15610), ab63+c51 (K15611), ab63+c52 (K15612), ab63+c53 (K15613), ab63+c54 (K15614), ab63+c55 (K15615), ab63+c56 (K15616), ab63+c57 (K15617), ab63+c58 (K15618), ab63+c59 (K15619), ab63+c60 (K15620), ab63+c61 (K15621), ab63+c62 (K15622), ab63+c63 (K15623), ab63+c64 (K15624), ab63+c65 (K15625), ab63+c66 (K15626), ab63+c67 (K15627), ab63+c68 (K15628), ab63+c69 (K15629), ab63+c70 (K15630), ab63+c71 (K15631), ab63+c72 (K15632), ab63+c73 (K15633), ab63+c74 (K15634), ab63+c75 (K15635), ab63+c76 (K15636), ab63+c77 (K15637), ab63+c78 (K15638), ab63+c79 (K15639), ab63+c80 (K15640), ab63+c81 (K15641), ab63+c82 (K15642), ab63+c83 (K15643), ab63+c84 (K15644), ab63+c85 (K15645), ab63+c86 (K15646), ab63+c87 (K15647), ab63+c88 (K15648), ab63+c89 (K15649), ab63+c90 (K15650), ab63+c91 (K15651), ab63+c92 (K15652), ab63+c93 (K15653), ab63+c94 (K15654), ab63+c95 (K15655), ab63+c96 (K15656), ab63+c97 (K15657), ab63+c98 (K15658), ab63+c99 (K15659), ab63+c100 (K15660), ab63+c101 (K15661), ab63+c102 (K15662), ab63+c103 (K15663), ab63+c104 (K15664), ab63+c105 (K15665), ab63+c106 (K15666), ab63+c107 (K15667), ab63+c108 (K15668), ab63+c109 (K15669), ab63+c110 (K15670), ab63+c111 (K15671), ab63+c112 (K15672), ab63+c113 (K15673), ab63+c114 (K15674), ab63+c115 (K15675), ab63+c116 (K15676), ab63+c117 (K15677), ab63+c118 (K15678), ab63+c119 (K15679), ab63+c120 (K15680), ab63+c121 (K15681), ab63+c122 (K15682), ab63+c123 (K15683), ab63+c124 (K15684), ab63+c125 (K15685), ab63+c126 (K15686), ab63+c127 (K15687), ab63+c128 (K15688), ab63+c129 (K15689), ab63+c130 (K15690), ab63+c131 (K15691), ab63+c132 (K15692), ab63+c133 (K15693), ab63+c134 (K15694), ab63+c135 (K15695), ab63+c136 (K15696), ab63+c137 (K15697), ab63+c138 (K15698), ab63+c139 (K15699), ab63+c140 (K15700), ab63+c141 (K15701), ab63+c142 (K15702), ab63+c143 (K15703), ab63+c144 (K15704), ab63+c145 (K15705), ab63+c146 (K15706), ab63+c147 (K15707), ab63+c148 (K15708), ab63+c149 (K15709), ab63+c150 (K15710), ab63+c151 (K15711), ab63+c152 (K15712), ab63+c153 (K15713), ab63+c154 (K15714), ab63+c155 (K15715), ab63+c156 (K15716), ab63+c157 (K15717), ab63+c158 (K15718), ab63+c159 (K15719), ab63+c160 (K15720), ab63+c161 (K15721), ab63+c162 (K15722), ab63+c163 (K15723), ab63+c164 (K15724), ab63+c165 (K15725), ab63+c166 (K15726), ab63+c167 (K15727), ab63+c168 (K15728), ab63+c169 (K15729), ab63+c170 (K15730), ab63+c171 (K15731), ab63+c172 (K15732), ab63+c173 (K15733), ab63+c174 (K15734), ab63+c175 (K15735), ab63+c176 (K15736), ab63+c177 (K15737), ab63+c178 (K15738), ab63+c179 (K15739), ab63+c180 (K15740), ab63+c181 (K15741), ab63+c182 (K15742), ab63+c183 (K15743), ab63+c184 (K15744), ab63+c185 (K15745), ab63+c186 (K15746), ab63+c187 (K15747), ab63+c188 (K15748), ab63+c189 (K15749), ab63+c190 (K15750), ab63+c191 (K15751), ab63+c192 (K15752), ab63+c193 (K15753), ab63+c194 (K15754), ab63+c195 (K15755), ab63+c196 (K15756), ab63+c197 (K15757), ab63+c198 (K15758), ab63+c199 (K15759), ab63+c200 (K15760), ab64+c1 (K15761), ab64+c2 (K15762), ab64+c3 (K15763), ab64+c4 (K15764), ab64+c5 (K15765), ab64+c6 (K15766), ab64+c7 (K15767), ab64+c8 (K15768), ab64+c9 (K15769), ab64+c10 (K15770), ab64+c11 (K15771), ab64+c12 (K15772), ab64+c13 (K15773), ab64+c14 (K15774), ab64+c15 (K15775), ab64+c16 (K15776), ab64+c17 (K15777), ab64+c18 (K15778), ab64+c19 (K15779), ab64+c20 (K15780), ab64+c21 (K15781), ab64+c22 (K15782), ab64+c23 (K15783), ab64+c24 (K15784), ab64+c25 (K15785), ab64+c26 (K15786), ab64+c27 (K15787), ab64+c28 (K15788), ab64+c29 (K15789), ab64+c30 (K15790), ab64+c31 (K15791), ab64+c32 (K15792), ab64+c33 (K15793), ab64+c34 (K15794), ab64+c35 (K15795), ab64+c36 (K15796), ab64+c37 (K15797), ab64+c38 (K15798), ab64+c39 (K15799), ab64+c40 (K15800), ab64+c41 (K15801), ab64+c42 (K15802), ab64+c43 (K15803), ab64+c44 (K15804), ab64+c45 (K15805), ab64+c46 (K15806), ab64+c47 (K15807), ab64+c48 (K15808), ab64+c49 (K15809), ab64+c50 (K15810), ab64+c51 (K15811), ab64+c52 (K15812), ab64+c53 (K15813), ab64+c54 (K15814), ab64+c55 (K15815), ab64+c56 (K15816), ab64+c57 (K15817), ab64+c58 (K15818), ab64+c59 (K15819), ab64+c60 (K15820), ab64+c61 (K15821), ab64+c62 (K15822), ab64+c63 (K15823), ab64+c64 (K15824), ab64+c65 (K15825), ab64+c66 (K15826), ab64+c67 (K15827), ab64+c68 (K15828), ab64+c69 (K15829), ab64+c70 (K15830), ab64+c71 (K15831), ab64+c72 (K15832), ab64+c73 (K15833), ab64+c74 (K15834), ab64+c75 (K15835), ab64+c76 (K15836), ab64+c77 (K15837), ab64+c78 (K15838), ab64+c79 (K15839), ab64+c80 (K15840), ab64+c81 (K15841), ab64+c82 (K15842), ab64+c83 (K15843), ab64+c84 (K15844), ab64+c85 (K15845), ab64+c86 (K15846), ab64+c87 (K15847), ab64+c88 (K15848), ab64+c89 (K15849), ab64+c90 (K15850), ab64+c91 (K15851), ab64+c92 (K15852), ab64+c93 (K15853), ab64+c94 (K15854), ab64+c95 (K15855), ab64+c96 (K15856), ab64+c97 (K15857), ab64+c98 (K15858), ab64+c99 (K15859), ab64+c100 (K15860), ab64+c101 (K15861), ab64+c102 (K15862), ab64+c103 (K15863), ab64+c104 (K15864), ab64+c105 (K15865), ab64+c106 (K15866), ab64+c107 (K15867), ab64+c108 (K15868), ab64+c109 (K15869), ab64+c110 (K15870), ab64+c111 (K15871), ab64+c112 (K15872), ab64+c113 (K15873), ab64+c114 (K15874), ab64+c115 (K15875), ab64+c116 (K15876), ab64+c117 (K15877), ab64+c118 (K15878), ab64+c119 (K15879), ab64+c120 (K15880), ab64+c121 (K15881), ab64+c122 (K15882), ab64+c123 (K15883), ab64+c124 (K15884), ab64+c125 (K15885), ab64+c126 (K15886), ab64+c127 (K15887), ab64+c128 (K15888), ab64+c129 (K15889), ab64+c130 (K15890), ab64+c131 (K15891), ab64+c132 (K15892), ab64+c133 (K15893), ab64+c134 (K15894), ab64+c135 (K15895), ab64+c136 (K15896), ab64+c137 (K15897), ab64+c138 (K15898), ab64+c139 (K15899), ab64+c140 (K15900), ab64+c141 (K15901), ab64+c142 (K15902), ab64+c143 (K15903), ab64+c144 (K15904), ab64+c145 (K15905), ab64+c146 (K15906), ab64+c147 (K15907), ab64+c148 (K15908), ab64+c149 (K15909), ab64+c150 (K15910), ab64+c151 (K15911), ab64+c152 (K15912), ab64+c153 (K15913), ab64+c154 (K15914), ab64+c155 (K15915), ab64+c156 (K15916), ab64+c157 (K15917), ab64+c158 (K15918), ab64+c159 (K15919), ab64+c160 (K15920), ab64+c161 (K15921), ab64+c162 (K15922), ab64+c163 (K15923), ab64+c164 (K15924), ab64+c165 (K15925), ab64+c166 (K15926), ab64+c167 (K15927), ab64+c168 (K15928), ab64+c169 (K15929), ab64+c170 (K15930), ab64+c171 (K15931), ab64+c172 (K15932), ab64+c173 (K15933), ab64+c174 (K15934), ab64+c175 (K15935), ab64+c176 (K15936), ab64+c177 (K15937), ab64+c178 (K15938), ab64+c179 (K15939), ab64+c180 (K15940), ab64+c181 (K15941), ab64+c182 (K15942), ab64+c183 (K15943), ab64+c184 (K15944), ab64+c185 (K15945), ab64+c186 (K15946), ab64+c187 (K15947), ab64+c188 (K15948), ab64+c189 (K15949), ab64+c190 (K15950), ab64+c191 (K15951), ab64+c192 (K15952), ab64+c193 (K15953), ab64+c194 (K15954), ab64+c195 (K15955), ab64+c196 (K15956), ab64+c197 (K15957), ab64+c198 (K15958), ab64+c199 (K15959), ab64+c200 (K15960), ab65+c1 (K15961), ab65+c2 (K15962), ab65+c3 (K15963), ab65+c4 (K15964), ab65+c5 (K15965), ab65+c6 (K15966), ab65+c7 (K15967), ab65+c8 (K15968), ab65+c9 (K15969), ab65+c10 (K15970), ab65+c11 (K15971), ab65+c12 (K15972), ab65+c13 (K15973), ab65+c14 (K15974), ab65+c15 (K15975), ab65+c16 (K15976), ab65+c17 (K15977), ab65+c18 (K15978), ab65+c19 (K15979), ab65+c20 (K15980), ab65+c21 (K15981), ab65+c22 (K15982), ab65+c23 (K15983), ab65+c24 (K15984), ab65+c25 (K15985), ab65+c26 (K15986), ab65+c27 (K15987), ab65+c28 (K15988), ab65+c29 (K15989), ab65+c30 (K15990), ab65+c31 (K15991), ab65+c32 (K15992), ab65+c33 (K15993), ab65+c34 (K15994), ab65+c35 (K15995), ab65+c36 (K15996), ab65+c37 (K15997), ab65+c38 (K15998), ab65+c39 (K15999), ab65+c40 (K16000), ab65+c41 (K16001), ab65+c42 (K16002), ab65+c43 (K16003), ab65+c44 (K16004), ab65+c45 (K16005), ab65+c46 (K16006), ab65+c47 (K16007), ab65+c48 (K16008), ab65+c49 (K16009), ab65+c50 (K16010), ab65+c51 (K16011), ab65+c52 (K16012), ab65+c53 (K16013), ab65+c54 (K16014), ab65+c55 (K16015), ab65+c56 (K16016), ab65+c57 (K16017), ab65+c58 (K16018), ab65+c59 (K16019), ab65+c60 (K16020), ab65+c61 (K16021), ab65+c62 (K16022), ab65+c63 (K16023), ab65+c64 (K16024), ab65+c65 (K16025), ab65+c66 (K16026), ab65+c67 (K16027), ab65+c68 (K16028), ab65+c69 (K16029), ab65+c70 (K16030), ab65+c71 (K16031), ab65+c72 (K16032), ab65+c73 (K16033), ab65+c74 (K16034), ab65+c75 (K16035), ab65+c76 (K16036), ab65+c77 (K16037), ab65+c78 (K16038), ab65+c79 (K16039), ab65+c80 (K16040), ab65+c81 (K16041), ab65+c82 (K16042), ab65+c83 (K16043), ab65+c84 (K16044), ab65+c85 (K16045), ab65+c86 (K16046), ab65+c87 (K16047), ab65+c88 (K16048), ab65+c89 (K16049), ab65+c90 (K16050), ab65+c91 (K16051), ab65+c92 (K16052), ab65+c93 (K16053), ab65+c94 (K16054), ab65+c95 (K16055), ab65+c96 (K16056), ab65+c97 (K16057), ab65+c98 (K16058), ab65+c99 (K16059), ab65+c100 (K16060), ab65+c101 (K16061), ab65+c102 (K16062), ab65+c103 (K16063), ab65+c104 (K16064), ab65+c105 (K16065), ab65+c106 (K16066), ab65+c107 (K16067), ab65+c108 (K16068), ab65+c109 (K16069), ab65+c110 (K16070), ab65+c111 (K16071), ab65+c112 (K16072), ab65+c113 (K16073), ab65+c114 (K16074), ab65+c115 (K16075), ab65+c116 (K16076), ab65+c117 (K16077), ab65+c118 (K16078), ab65+c119 (K16079), ab65+c120 (K16080), ab65+c121 (K16081), ab65+c122 (K16082), ab65+c123 (K16083), ab65+c124 (K16084), ab65+c125 (K16085), ab65+c126 (K16086), ab65+c127 (K16087), ab65+c128 (K16088), ab65+c129 (K16089), ab65+c130 (K16090), ab65+c131 (K16091), ab65+c132 (K16092), ab65+c133 (K16093), ab65+c134 (K16094), ab65+c135 (K16095), ab65+c136 (K16096), ab65+c137 (K16097), ab65+c138 (K16098), ab65+c139 (K16099), ab65+c140 (K16100), ab65+c141 (K16101), ab65+c142 (K16102), ab65+c143 (K16103), ab65+c144 (K16104), ab65+c145 (K16105), ab65+c146 (K16106), ab65+c147 (K16107), ab65+c148 (K16108), ab65+c149 (K16109), ab65+c150 (K16110), ab65+c151 (K16111), ab65+c152 (K16112), ab65+c153 (K16113), ab65+c154 (K16114), ab65+c155 (K16115), ab65+c156 (K16116), ab65+c157 (K16117), ab65+c158 (K16118), ab65+c159 (K16119), ab65+c160 (K16120), ab65+c161 (K16121), ab65+c162 (K16122), ab65+c163 (K16123), ab65+c164 (K16124), ab65+c165 (K16125), ab65+c166 (K16126), ab65+c167 (K16127), ab65+c168 (K16128), ab65+c169 (K16129), ab65+c170 (K16130), ab65+c171 (K16131), ab65+c172 (K16132), ab65+c173 (K16133), ab65+c174 (K16134), ab65+c175 (K16135), ab65+c176 (K16136), ab65+c177 (K16137), ab65+c178 (K16138), ab65+c179 (K16139), ab65+c180 (K16140), ab65+c181 (K16141), ab65+c182 (K16142), ab65+c183 (K16143), ab65+c184 (K16144), ab65+c185 (K16145), ab65+c186 (K16146), ab65+c187 (K16147), ab65+c188 (K16148), ab65+c189 (K16149), ab65+c190 (K16150), ab65+c191 (K16151), ab65+c192 (K16152), ab65+c193 (K16153), ab65+c194 (K16154), ab65+c195 (K16155), ab65+c196 (K16156), ab65+c197 (K16157), ab65+c198 (K16158), ab65+c199 (K16159), ab65+c200 (K16160), ab66+c1 (K16161), ab66+c2 (K16162), ab66+c3 (K16163), ab66+c4 (K16164), ab66+c5 (K16165), ab66+c6 (K16166), ab66+c7 (K16167), ab66+c8 (K16168), ab66+c9 (K16169), ab66+c10 (K16170), ab66+c11 (K16171), ab66+c12 (K16172), ab66+c13 (K16173), ab66+c14 (K16174), ab66+c15 (K16175), ab66+c16 (K16176), ab66+c17 (K16177), ab66+c18 (K16178), ab66+c19 (K16179), ab66+c20 (K16180), ab66+c21 (K16181), ab66+c22 (K16182), ab66+c23 (K16183), ab66+c24 (K16184), ab66+c25 (K16185), ab66+c26 (K16186), ab66+c27 (K16187), ab66+c28 (K16188), ab66+c29 (K16189), ab66+c30 (K16190), ab66+c31 (K16191), ab66+c32 (K16192), ab66+c33 (K16193), ab66+c34 (K16194), ab66+c35 (K16195), ab66+c36 (K16196), ab66+c37 (K16197), ab66+c38 (K16198), ab66+c39 (K16199), ab66+c40 (K16200), ab66+c41 (K16201), ab66+c42 (K16202), ab66+c43 (K16203), ab66+c44 (K16204), ab66+c45 (K16205), ab66+c46 (K16206), ab66+c47 (K16207), ab66+c48 (K16208), ab66+c49 (K16209), ab66+c50 (K16210), ab66+c51 (K16211), ab66+c52 (K16212), ab66+c53 (K16213), ab66+c54 (K16214), ab66+c55 (K16215), ab66+c56 (K16216), ab66+c57 (K16217), ab66+c58 (K16218), ab66+c59 (K16219), ab66+c60 (K16220), ab66+c61 (K16221), ab66+c62 (K16222), ab66+c63 (K16223), ab66+c64 (K16224), ab66+c65 (K16225), ab66+c66 (K16226), ab66+c67 (K16227), ab66+c68 (K16228), ab66+c69 (K16229), ab66+c70 (K16230), ab66+c71 (K16231), ab66+c72 (K16232), ab66+c73 (K16233), ab66+c74 (K16234), ab66+c75 (K16235), ab66+c76 (K16236), ab66+c77 (K16237), ab66+c78 (K16238), ab66+c79 (K16239), ab66+c80 (K16240), ab66+c81 (K16241), ab66+c82 (K16242), ab66+c83 (K16243), ab66+c84 (K16244), ab66+c85 (K16245), ab66+c86 (K16246), ab66+c87 (K16247), ab66+c88 (K16248), ab66+c89 (K16249), ab66+c90 (K16250), ab66+c91 (K16251), ab66+c92 (K16252), ab66+c93 (K16253), ab66+c94 (K16254), ab66+c95 (K16255), ab66+c96 (K16256), ab66+c97 (K16257), ab66+c98 (K16258), ab66+c99 (K16259), ab66+c100 (K16260), ab66+c101 (K16261), ab66+c102 (K16262), ab66+c103 (K16263), ab66+c104 (K16264), ab66+c105 (K16265), ab66+c106 (K16266), ab66+c107 (K16267), ab66+c108 (K16268), ab66+c109 (K16269), ab66+c110 (K16270), ab66+c111 (K16271), ab66+c112 (K16272), ab66+c113 (K16273), ab66+c114 (K16274), ab66+c115 (K16275), ab66+c116 (K16276), ab66+c117 (K16277), ab66+c118 (K16278), ab66+c119 (K16279), ab66+c120 (K16280), ab66+c121 (K16281), ab66+c122 (K16282), ab66+c123 (K16283), ab66+c124 (K16284), ab66+c125 (K16285), ab66+c126 (K16286), ab66+c127 (K16287), ab66+c128 (K16288), ab66+c129 (K16289), ab66+c130 (K16290), ab66+c131 (K16291), ab66+c132 (K16292), ab66+c133 (K16293), ab66+c134 (K16294), ab66+c135 (K16295), ab66+c136 (K16296), ab66+c137 (K16297), ab66+c138 (K16298), ab66+c139 (K16299), ab66+c140 (K16300), ab66+c141 (K16301), ab66+c142 (K16302), ab66+c143 (K16303), ab66+c144 (K16304), ab66+c145 (K16305), ab66+c146 (K16306), ab66+c147 (K16307), ab66+c148 (K16308), ab66+c149 (K16309), ab66+c150 (K16310), ab66+c151 (K16311), ab66+c152 (K16312), ab66+c153 (K16313), ab66+c154 (K16314), ab66+c155 (K16315), ab66+c156 (K16316), ab66+c157 (K16317), ab66+c158 (K16318), ab66+c159 (K16319), ab66+c160 (K16320), ab66+c161 (K16321), ab66+c162 (K16322), ab66+c163 (K16323), ab66+c164 (K16324), ab66+c165 (K16325), ab66+c166 (K16326), ab66+c167 (K16327), ab66+c168 (K16328), ab66+c169 (K16329), ab66+c170 (K16330), ab66+c171 (K16331), ab66+c172 (K16332), ab66+c173 (K16333), ab66+c174 (K16334), ab66+c175 (K16335), ab66+c176 (K16336), ab66+c177 (K16337), ab66+c178 (K16338), ab66+c179 (K16339), ab66+c180 (K16340), ab66+c181 (K16341), ab66+c182 (K16342), ab66+c183 (K16343), ab66+c184 (K16344), ab66+c185 (K16345), ab66+c186 (K16346), ab66+c187 (K16347), ab66+c188 (K16348), ab66+c189 (K16349), ab66+c190 (K16350), ab66+c191 (K16351), ab66+c192 (K16352), ab66+c193 (K16353), ab66+c194 (K16354), ab66+c195 (K16355), ab66+c196 (K16356), ab66+c197 (K16357), ab66+c198 (K16358), ab66+c199 (K16359), ab66+c200 (K16360), ab67+c1 (K16361), ab67+c2 (K16362), ab67+c3 (K16363), ab67+c4 (K16364), ab67+c5 (K16365), ab67+c6 (K16366), ab67+c7 (K16367), ab67+c8 (K16368), ab67+c9 (K16369), ab67+c10 (K16370), ab67+c11 (K16371), ab67+c12 (K16372), ab67+c13 (K16373), ab67+c14 (K16374), ab67+c15 (K16375), ab67+c16 (K16376), ab67+c17 (K16377), ab67+c18 (K16378), ab67+c19 (K16379), ab67+c20 (K16380), ab67+c21 (K16381), ab67+c22 (K16382), ab67+c23 (K16383), ab67+c24 (K16384), ab67+c25 (K16385), ab67+c26 (K16386), ab67+c27 (K16387), ab67+c28 (K16388), ab67+c29 (K16389), ab67+c30 (K16390), ab67+c31 (K16391), ab67+c32 (K16392), ab67+c33 (K16393), ab67+c34 (K16394), ab67+c35 (K16395), ab67+c36 (K16396), ab67+c37 (K16397), ab67+c38 (K16398), ab67+c39 (K16399), ab67+c40 (K16400), ab67+c41 (K16401), ab67+c42 (K16402), ab67+c43 (K16403), ab67+c44 (K16404), ab67+c45 (K16405), ab67+c46 (K16406), ab67+c47 (K16407), ab67+c48 (K16408), ab67+c49 (K16409), ab67+c50 (K16410), ab67+c51 (K16411), ab67+c52 (K16412), ab67+c53 (K16413), ab67+c54 (K16414), ab67+c55 (K16415), ab67+c56 (K16416), ab67+c57 (K16417), ab67+c58 (K16418), ab67+c59 (K16419), ab67+c60 (K16420), ab67+c61 (K16421), ab67+c62 (K16422), ab67+c63 (K16423), ab67+c64 (K16424), ab67+c65 (K16425), ab67+c66 (K16426), ab67+c67 (K16427), ab67+c68 (K16428), ab67+c69 (K16429), ab67+c70 (K16430), ab67+c71 (K16431), ab67+c72 (K16432), ab67+c73 (K16433), ab67+c74 (K16434), ab67+c75 (K16435), ab67+c76 (K16436), ab67+c77 (K16437), ab67+c78 (K16438), ab67+c79 (K16439), ab67+c80 (K16440), ab67+c81 (K16441), ab67+c82 (K16442), ab67+c83 (K16443), ab67+c84 (K16444), ab67+c85 (K16445), ab67+c86 (K16446), ab67+c87 (K16447), ab67+c88 (K16448), ab67+c89 (K16449), ab67+c90 (K16450), ab67+c91 (K16451), ab67+c92 (K16452), ab67+c93 (K16453), ab67+c94 (K16454), ab67+c95 (K16455), ab67+c96 (K16456), ab67+c97 (K16457), ab67+c98 (K16458), ab67+c99 (K16459), ab67+c100 (K16460), ab67+c101 (K16461), ab67+c102 (K16462), ab67+c103 (K16463), ab67+c104 (K16464), ab67+c105 (K16465), ab67+c106 (K16466), ab67+c107 (K16467), ab67+c108 (K16468), ab67+c109 (K16469), ab67+c110 (K16470), ab67+c111 (K16471), ab67+c112 (K16472), ab67+c113 (K16473), ab67+c114 (K16474), ab67+c115 (K16475), ab67+c116 (K16476), ab67+c117 (K16477), ab67+c118 (K16478), ab67+c119 (K16479), ab67+c120 (K16480), ab67+c121 (K16481), ab67+c122 (K16482), ab67+c123 (K16483), ab67+c124 (K16484), ab67+c125 (K16485), ab67+c126 (K16486), ab67+c127 (K16487), ab67+c128 (K16488), ab67+c129 (K16489), ab67+c130 (K16490), ab67+c131 (K16491), ab67+c132 (K16492), ab67+c133 (K16493), ab67+c134 (K16494), ab67+c135 (K16495), ab67+c136 (K16496), ab67+c137 (K16497), ab67+c138 (K16498), ab67+c139 (K16499), ab67+c140 (K16500), ab67+c141 (K16501), ab67+c142 (K16502), ab67+c143 (K16503), ab67+c144 (K16504), ab67+c145 (K16505), ab67+c146 (K16506), ab67+c147 (K16507), ab67+c148 (K16508), ab67+c149 (K16509), ab67+c150 (K16510), ab67+c151 (K16511), ab67+c152 (K16512), ab67+c153 (K16513), ab67+c154 (K16514), ab67+c155 (K16515), ab67+c156 (K16516), ab67+c157 (K16517), ab67+c158 (K16518), ab67+c159 (K16519), ab67+c160 (K16520), ab67+c161 (K16521), ab67+c162 (K16522), ab67+c163 (K16523), ab67+c164 (K16524), ab67+c165 (K16525), ab67+c166 (K16526), ab67+c167 (K16527), ab67+c168 (K16528), ab67+c169 (K16529), ab67+c170 (K16530), ab67+c171 (K16531), ab67+c172 (K16532), ab67+c173 (K16533), ab67+c174 (K16534), ab67+c175 (K16535), ab67+c176 (K16536), ab67+c177 (K16537), ab67+c178 (K16538), ab67+c179 (K16539), ab67+c180 (K16540), ab67+c181 (K16541), ab67+c182 (K16542), ab67+c183 (K16543), ab67+c184 (K16544), ab67+c185 (K16545), ab67+c186 (K16546), ab67+c187 (K16547), ab67+c188 (K16548), ab67+c189 (K16549), ab67+c190 (K16550), ab67+c191 (K16551), ab67+c192 (K16552), ab67+c193 (K16553), ab67+c194 (K16554), ab67+c195 (K16555), ab67+c196 (K16556), ab67+c197 (K16557), ab67+c198 (K16558), ab67+c199 (K16559), ab67+c200 (K16560), ab68+c1 (K16561), ab68+c2 (K16562), ab68+c3 (K16563), ab68+c4 (K16564), ab68+c5 (K16565), ab68+c6 (K16566), ab68+c7 (K16567), ab68+c8 (K16568), ab68+c9 (K16569), ab68+c10 (K16570), ab68+c11 (K16571), ab68+c12 (K16572), ab68+c13 (K16573), ab68+c14 (K16574), ab68+c15 (K16575), ab68+c16 (K16576), ab68+c17 (K16577), ab68+c18 (K16578), ab68+c19 (K16579), ab68+c20 (K16580), ab68+c21 (K16581), ab68+c22 (K16582), ab68+c23 (K16583), ab68+c24 (K16584), ab68+c25 (K16585), ab68+c26 (K16586), ab68+c27 (K16587), ab68+c28 (K16588), ab68+c29 (K16589), ab68+c30 (K16590), ab68+c31 (K16591), ab68+c32 (K16592), ab68+c33 (K16593), ab68+c34 (K16594), ab68+c35 (K16595), ab68+c36 (K16596), ab68+c37 (K16597), ab68+c38 (K16598), ab68+c39 (K16599), ab68+c40 (K16600), ab68+c41 (K16601), ab68+c42 (K16602), ab68+c43 (K16603), ab68+c44 (K16604), ab68+c45 (K16605), ab68+c46 (K16606), ab68+c47 (K16607), ab68+c48 (K16608), ab68+c49 (K16609), ab68+c50 (K16610), ab68+c51 (K16611), ab68+c52 (K16612), ab68+c53 (K16613), ab68+c54 (K16614), ab68+c55 (K16615), ab68+c56 (K16616), ab68+c57 (K16617), ab68+c58 (K16618), ab68+c59 (K16619), ab68+c60 (K16620), ab68+c61 (K16621), ab68+c62 (K16622), ab68+c63 (K16623), ab68+c64 (K16624), ab68+c65 (K16625), ab68+c66 (K16626), ab68+c67 (K16627), ab68+c68 (K16628), ab68+c69 (K16629), ab68+c70 (K16630), ab68+c71 (K16631), ab68+c72 (K16632), ab68+c73 (K16633), ab68+c74 (K16634), ab68+c75 (K16635), ab68+c76 (K16636), ab68+c77 (K16637), ab68+c78 (K16638), ab68+c79 (K16639), ab68+c80 (K16640), ab68+c81 (K16641), ab68+c82 (K16642), ab68+c83 (K16643), ab68+c84 (K16644), ab68+c85 (K16645), ab68+c86 (K16646), ab68+c87 (K16647), ab68+c88 (K16648), ab68+c89 (K16649), ab68+c90 (K16650), ab68+c91 (K16651), ab68+c92 (K16652), ab68+c93 (K16653), ab68+c94 (K16654), ab68+c95 (K16655), ab68+c96 (K16656), ab68+c97 (K16657), ab68+c98 (K16658), ab68+c99 (K16659), ab68+c100 (K16660), ab68+c101 (K16661), ab68+c102 (K16662), ab68+c103 (K16663), ab68+c104 (K16664), ab68+c105 (K16665), ab68+c106 (K16666), ab68+c107 (K16667), ab68+c108 (K16668), ab68+c109 (K16669), ab68+c110 (K16670), ab68+c111 (K16671), ab68+c112 (K16672), ab68+c113 (K16673), ab68+c114 (K16674), ab68+c115 (K16675), ab68+c116 (K16676), ab68+c117 (K16677), ab68+c118 (K16678), ab68+c119 (K16679), ab68+c120 (K16680), ab68+c121 (K16681), ab68+c122 (K16682), ab68+c123 (K16683), ab68+c124 (K16684), ab68+c125 (K16685), ab68+c126 (K16686), ab68+c127 (K16687), ab68+c128 (K16688), ab68+c129 (K16689), ab68+c130 (K16690), ab68+c131 (K16691), ab68+c132 (K16692), ab68+c133 (K16693), ab68+c134 (K16694), ab68+c135 (K16695), ab68+c136 (K16696), ab68+c137 (K16697), ab68+c138 (K16698), ab68+c139 (K16699), ab68+c140 (K16700), ab68+c141 (K16701), ab68+c142 (K16702), ab68+c143 (K16703), ab68+c144 (K16704), ab68+c145 (K16705), ab68+c146 (K16706), ab68+c147 (K16707), ab68+c148 (K16708), ab68+c149 (K16709), ab68+c150 (K16710), ab68+c151 (K16711), ab68+c152 (K16712), ab68+c153 (K16713), ab68+c154 (K16714), ab68+c155 (K16715), ab68+c156 (K16716), ab68+c157 (K16717), ab68+c158 (K16718), ab68+c159 (K16719), ab68+c160 (K16720), ab68+c161 (K16721), ab68+c162 (K16722), ab68+c163 (K16723), ab68+c164 (K16724), ab68+c165 (K16725), ab68+c166 (K16726), ab68+c167 (K16727), ab68+c168 (K16728), ab68+c169 (K16729), ab68+c170 (K16730), ab68+c171 (K16731), ab68+c172 (K16732), ab68+c173 (K16733), ab68+c174 (K16734), ab68+c175 (K16735), ab68+c176 (K16736), ab68+c177 (K16737), ab68+c178 (K16738), ab68+c179 (K16739), ab68+c180 (K16740), ab68+c181 (K16741), ab68+c182 (K16742), ab68+c183 (K16743), ab68+c184 (K16744), ab68+c185 (K16745), ab68+c186 (K16746), ab68+c187 (K16747), ab68+c188 (K16748), ab68+c189 (K16749), ab68+c190 (K16750), ab68+c191 (K16751), ab68+c192 (K16752), ab68+c193 (K16753), ab68+c194 (K16754), ab68+c195 (K16755), ab68+c196 (K16756), ab68+c197 (K16757), ab68+c198 (K16758), ab68+c199 (K16759), ab68+c200 (K16760), ab69+c1 (K16761), ab69+c2 (K16762), ab69+c3 (K16763), ab69+c4 (K16764), ab69+c5 (K16765), ab69+c6 (K16766), ab69+c7 (K16767), ab69+c8 (K16768), ab69+c9 (K16769), ab69+c10 (K16770), ab69+c11 (K16771), ab69+c12 (K16772), ab69+c13 (K16773), ab69+c14 (K16774), ab69+c15 (K16775), ab69+c16 (K16776), ab69+c17 (K16777), ab69+c18 (K16778), ab69+c19 (K16779), ab69+c20 (K16780), ab69+c21 (K16781), ab69+c22 (K16782), ab69+c23 (K16783), ab69+c24 (K16784), ab69+c25 (K16785), ab69+c26 (K16786), ab69+c27 (K16787), ab69+c28 (K16788), ab69+c29 (K16789), ab69+c30 (K16790), ab69+c31 (K16791), ab69+c32 (K16792), ab69+c33 (K16793), ab69+c34 (K16794), ab69+c35 (K16795), ab69+c36 (K16796), ab69+c37 (K16797), ab69+c38 (K16798), ab69+c39 (K16799), ab69+c40 (K16800), ab69+c41 (K16801), ab69+c42 (K16802), ab69+c43 (K16803), ab69+c44 (K16804), ab69+c45 (K16805), ab69+c46 (K16806), ab69+c47 (K16807), ab69+c48 (K16808), ab69+c49 (K16809), ab69+c50 (K16810), ab69+c51 (K16811), ab69+c52 (K16812), ab69+c53 (K16813), ab69+c54 (K16814), ab69+c55 (K16815), ab69+c56 (K16816), ab69+c57 (K16817), ab69+c58 (K16818), ab69+c59 (K16819), ab69+c60 (K16820), ab69+c61 (K16821), ab69+c62 (K16822), ab69+c63 (K16823), ab69+c64 (K16824), ab69+c65 (K16825), ab69+c66 (K16826), ab69+c67 (K16827), ab69+c68 (K16828), ab69+c69 (K16829), ab69+c70 (K16830), ab69+c71 (K16831), ab69+c72 (K16832), ab69+c73 (K16833), ab69+c74 (K16834), ab69+c75 (K16835), ab69+c76 (K16836), ab69+c77 (K16837), ab69+c78 (K16838), ab69+c79 (K16839), ab69+c80 (K16840), ab69+c81 (K16841), ab69+c82 (K16842), ab69+c83 (K16843), ab69+c84 (K16844), ab69+c85 (K16845), ab69+c86 (K16846), ab69+c87 (K16847), ab69+c88 (K16848), ab69+c89 (K16849), ab69+c90 (K16850), ab69+c91 (K16851), ab69+c92 (K16852), ab69+c93 (K16853), ab69+c94 (K16854), ab69+c95 (K16855), ab69+c96 (K16856), ab69+c97 (K16857), ab69+c98 (K16858), ab69+c99 (K16859), ab69+c100 (K16860), ab69+c101 (K16861), ab69+c102 (K16862), ab69+c103 (K16863), ab69+c104 (K16864), ab69+c105 (K16865), ab69+c106 (K16866), ab69+c107 (K16867), ab69+c108 (K16868), ab69+c109 (K16869), ab69+c110 (K16870), ab69+c111 (K16871), ab69+c112 (K16872), ab69+c113 (K16873), ab69+c114 (K16874), ab69+c115 (K16875), ab69+c116 (K16876), ab69+c117 (K16877), ab69+c118 (K16878), ab69+c119 (K16879), ab69+c120 (K16880), ab69+c121 (K16881), ab69+c122 (K16882), ab69+c123 (K16883), ab69+c124 (K16884), ab69+c125 (K16885), ab69+c126 (K16886), ab69+c127 (K16887), ab69+c128 (K16888), ab69+c129 (K16889), ab69+c130 (K16890), ab69+c131 (K16891), ab69+c132 (K16892), ab69+c133 (K16893), ab69+c134 (K16894), ab69+c135 (K16895), ab69+c136 (K16896), ab69+c137 (K16897), ab69+c138 (K16898), ab69+c139 (K16899), ab69+c140 (K16900), ab69+c141 (K16901), ab69+c142 (K16902), ab69+c143 (K16903), ab69+c144 (K16904), ab69+c145 (K16905), ab69+c146 (K16906), ab69+c147 (K16907), ab69+c148 (K16908), ab69+c149 (K16909), ab69+c150 (K16910), ab69+c151 (K16911), ab69+c152 (K16912), ab69+c153 (K16913), ab69+c154 (K16914), ab69+c155 (K16915), ab69+c156 (K16916), ab69+c157 (K16917), ab69+c158 (K16918), ab69+c159 (K16919), ab69+c160 (K16920), ab69+c161 (K16921), ab69+c162 (K16922), ab69+c163 (K16923), ab69+c164 (K16924), ab69+c165 (K16925), ab69+c166 (K16926), ab69+c167 (K16927), ab69+c168 (K16928), ab69+c169 (K16929), ab69+c170 (K16930), ab69+c171 (K16931), ab69+c172 (K16932), ab69+c173 (K16933), ab69+c174 (K16934), ab69+c175 (K16935), ab69+c176 (K16936), ab69+c177 (K16937), ab69+c178 (K16938), ab69+c179 (K16939), ab69+c180 (K16940), ab69+c181 (K16941), ab69+c182 (K16942), ab69+c183 (K16943), ab69+c184 (K16944), ab69+c185 (K16945), ab69+c186 (K16946), ab69+c187 (K16947), ab69+c188 (K16948), ab69+c189 (K16949), ab69+c190 (K16950), ab69+c191 (K16951), ab69+c192 (K16952), ab69+c193 (K16953), ab69+c194 (K16954), ab69+c195 (K16955), ab69+c196 (K16956), ab69+c197 (K16957), ab69+c198 (K16958), ab69+c199 (K16959), ab69+c200 (K16960), ab70+c1 (K16961), ab70+c2 (K16962), ab70+c3 (K16963), ab70+c4 (K16964), ab70+c5 (K16965), ab70+c6 (K16966), ab70+c7 (K16967), ab70+c8 (K16968), ab70+c9 (K16969), ab70+c10 (K16970), ab70+c11 (K16971), ab70+c12 (K16972), ab70+c13 (K16973), ab70+c14 (K16974), ab70+c15 (K16975), ab70+c16 (K16976), ab70+c17 (K16977), ab70+c18 (K16978), ab70+c19 (K16979), ab70+c20 (K16980), ab70+c21 (K16981), ab70+c22 (K16982), ab70+c23 (K16983), ab70+c24 (K16984), ab70+c25 (K16985), ab70+c26 (K16986), ab70+c27 (K16987), ab70+c28 (K16988), ab70+c29 (K16989), ab70+c30 (K16990), ab70+c31 (K16991), ab70+c32 (K16992), ab70+c33 (K16993), ab70+c34 (K16994), ab70+c35 (K16995), ab70+c36 (K16996), ab70+c37 (K16997), ab70+c38 (K16998), ab70+c39 (K16999), ab70+c40 (K17000), ab70+c41 (K17001), ab70+c42 (K17002), ab70+c43 (K17003), ab70+c44 (K17004), ab70+c45 (K17005), ab70+c46 (K17006), ab70+c47 (K17007), ab70+c48 (K17008), ab70+c49 (K17009), ab70+c50 (K17010), ab70+c51 (K17011), ab70+c52 (K17012), ab70+c53 (K17013), ab70+c54 (K17014), ab70+c55 (K17015), ab70+c56 (K17016), ab70+c57 (K17017), ab70+c58 (K17018), ab70+c59 (K17019), ab70+c60 (K17020), ab70+c61 (K17021), ab70+c62 (K17022), ab70+c63 (K17023), ab70+c64 (K17024), ab70+c65 (K17025), ab70+c66 (K17026), ab70+c67 (K17027), ab70+c68 (K17028), ab70+c69 (K17029), ab70+c70 (K17030), ab70+c71 (K17031), ab70+c72 (K17032), ab70+c73 (K17033), ab70+c74 (K17034), ab70+c75 (K17035), ab70+c76 (K17036), ab70+c77 (K17037), ab70+c78 (K17038), ab70+c79 (K17039), ab70+c80 (K17040), ab70+c81 (K17041), ab70+c82 (K17042), ab70+c83 (K17043), ab70+c84 (K17044), ab70+c85 (K17045), ab70+c86 (K17046), ab70+c87 (K17047), ab70+c88 (K17048), ab70+c89 (K17049), ab70+c90 (K17050), ab70+c91 (K17051), ab70+c92 (K17052), ab70+c93 (K17053), ab70+c94 (K17054), ab70+c95 (K17055), ab70+c96 (K17056), ab70+c97 (K17057), ab70+c98 (K17058), ab70+c99 (K17059), ab70+c100 (K17060), ab70+c101 (K17061), ab70+c102 (K17062), ab70+c103 (K17063), ab70+c104 (K17064), ab70+c105 (K17065), ab70+c106 (K17066), ab70+c107 (K17067), ab70+c108 (K17068), ab70+c109 (K17069), ab70+c110 (K17070), ab70+c111 (K17071), ab70+c112 (K17072), ab70+c113 (K17073), ab70+c114 (K17074), ab70+c115 (K17075), ab70+c116 (K17076), ab70+c117 (K17077), ab70+c118 (K17078), ab70+c119 (K17079), ab70+c120 (K17080), ab70+c121 (K17081), ab70+c122 (K17082), ab70+c123 (K17083), ab70+c124 (K17084), ab70+c125 (K17085), ab70+c126 (K17086), ab70+c127 (K17087), ab70+c128 (K17088), ab70+c129 (K17089), ab70+c130 (K17090), ab70+c131 (K17091), ab70+c132 (K17092), ab70+c133 (K17093), ab70+c134 (K17094), ab70+c135 (K17095), ab70+c136 (K17096), ab70+c137 (K17097), ab70+c138 (K17098), ab70+c139 (K17099), ab70+c140 (K17100), ab70+c141 (K17101), ab70+c142 (K17102), ab70+c143 (K17103), ab70+c144 (K17104), ab70+c145 (K17105), ab70+c146 (K17106), ab70+c147 (K17107), ab70+c148 (K17108), ab70+c149 (K17109), ab70+c150 (K17110), ab70+c151 (K17111), ab70+c152 (K17112), ab70+c153 (K17113), ab70+c154 (K17114), ab70+c155 (K17115), ab70+c156 (K17116), ab70+c157 (K17117), ab70+c158 (K17118), ab70+c159 (K17119), ab70+c160 (K17120), ab70+c161 (K17121), ab70+c162 (K17122), ab70+c163 (K17123), ab70+c164 (K17124), ab70+c165 (K17125), ab70+c166 (K17126), ab70+c167 (K17127), ab70+c168 (K17128), ab70+c169 (K17129), ab70+c170 (K17130), ab70+c171 (K17131), ab70+c172 (K17132), ab70+c173 (K17133), ab70+c174 (K17134), ab70+c175 (K17135), ab70+c176 (K17136), ab70+c177 (K17137), ab70+c178 (K17138), ab70+c179 (K17139), ab70+c180 (K17140), ab70+c181 (K17141), ab70+c182 (K17142), ab70+c183 (K17143), ab70+c184 (K17144), ab70+c185 (K17145), ab70+c186 (K17146), ab70+c187 (K17147), ab70+c188 (K17148), ab70+c189 (K17149), ab70+c190 (K17150), ab70+c191 (K17151), ab70+c192 (K17152), ab70+c193 (K17153), ab70+c194 (K17154), ab70+c195 (K17155), ab70+c196 (K17156), ab70+c197 (K17157), ab70+c198 (K17158), ab70+c199 (K17159), ab70+c200 (K17160), ab71+c1 (K17161), ab71+c2 (K17162), ab71+c3 (K17163), ab71+c4 (K17164), ab71+c5 (K17165), ab71+c6 (K17166), ab71+c7 (K17167), ab71+c8 (K17168), ab71+c9 (K17169), ab71+c10 (K17170), ab71+c11 (K17171), ab71+c12 (K17172), ab71+c13 (K17173), ab71+c14 (K17174), ab71+c15 (K17175), ab71+c16 (K17176), ab71+c17 (K17177), ab71+c18 (K17178), ab71+c19 (K17179), ab71+c20 (K17180), ab71+c21 (K17181), ab71+c22 (K17182), ab71+c23 (K17183), ab71+c24 (K17184), ab71+c25 (K17185), ab71+c26 (K17186), ab71+c27 (K17187), ab71+c28 (K17188), ab71+c29 (K17189), ab71+c30 (K17190), ab71+c31 (K17191), ab71+c32 (K17192), ab71+c33 (K17193), ab71+c34 (K17194), ab71+c35 (K17195), ab71+c36 (K17196), ab71+c37 (K17197), ab71+c38 (K17198), ab71+c39 (K17199), ab71+c40 (K17200), ab71+c41 (K17201), ab71+c42 (K17202), ab71+c43 (K17203), ab71+c44 (K17204), ab71+c45 (K17205), ab71+c46 (K17206), ab71+c47 (K17207), ab71+c48 (K17208), ab71+c49 (K17209), ab71+c50 (K17210), ab71+c51 (K17211), ab71+c52 (K17212), ab71+c53 (K17213), ab71+c54 (K17214), ab71+c55 (K17215), ab71+c56 (K17216), ab71+c57 (K17217), ab71+c58 (K17218), ab71+c59 (K17219), ab71+c60 (K17220), ab71+c61 (K17221), ab71+c62 (K17222), ab71+c63 (K17223), ab71+c64 (K17224), ab71+c65 (K17225), ab71+c66 (K17226), ab71+c67 (K17227), ab71+c68 (K17228), ab71+c69 (K17229), ab71+c70 (K17230), ab71+c71 (K17231), ab71+c72 (K17232), ab71+c73 (K17233), ab71+c74 (K17234), ab71+c75 (K17235), ab71+c76 (K17236), ab71+c77 (K17237), ab71+c78 (K17238), ab71+c79 (K17239), ab71+c80 (K17240), ab71+c81 (K17241), ab71+c82 (K17242), ab71+c83 (K17243), ab71+c84 (K17244), ab71+c85 (K17245), ab71+c86 (K17246), ab71+c87 (K17247), ab71+c88 (K17248), ab71+c89 (K17249), ab71+c90 (K17250), ab71+c91 (K17251), ab71+c92 (K17252), ab71+c93 (K17253), ab71+c94 (K17254), ab71+c95 (K17255), ab71+c96 (K17256), ab71+c97 (K17257), ab71+c98 (K17258), ab71+c99 (K17259), ab71+c100 (K17260), ab71+c101 (K17261), ab71+c102 (K17262), ab71+c103 (K17263), ab71+c104 (K17264), ab71+c105 (K17265), ab71+c106 (K17266), ab71+c107 (K17267), ab71+c108 (K17268), ab71+c109 (K17269), ab71+c110 (K17270), ab71+c111 (K17271), ab71+c112 (K17272), ab71+c113 (K17273), ab71+c114 (K17274), ab71+c115 (K17275), ab71+c116 (K17276), ab71+c117 (K17277), ab71+c118 (K17278), ab71+c119 (K17279), ab71+c120 (K17280), ab71+c121 (K17281), ab71+c122 (K17282), ab71+c123 (K17283), ab71+c124 (K17284), ab71+c125 (K17285), ab71+c126 (K17286), ab71+c127 (K17287), ab71+c128 (K17288), ab71+c129 (K17289), ab71+c130 (K17290), ab71+c131 (K17291), ab71+c132 (K17292), ab71+c133 (K17293), ab71+c134 (K17294), ab71+c135 (K17295), ab71+c136 (K17296), ab71+c137 (K17297), ab71+c138 (K17298), ab71+c139 (K17299), ab71+c140 (K17300), ab71+c141 (K17301), ab71+c142 (K17302), ab71+c143 (K17303), ab71+c144 (K17304), ab71+c145 (K17305), ab71+c146 (K17306), ab71+c147 (K17307), ab71+c148 (K17308), ab71+c149 (K17309), ab71+c150 (K17310), ab71+c151 (K17311), ab71+c152 (K17312), ab71+c153 (K17313), ab71+c154 (K17314), ab71+c155 (K17315), ab71+c156 (K17316), ab71+c157 (K17317), ab71+c158 (K17318), ab71+c159 (K17319), ab71+c160 (K17320), ab71+c161 (K17321), ab71+c162 (K17322), ab71+c163 (K17323), ab71+c164 (K17324), ab71+c165 (K17325), ab71+c166 (K17326), ab71+c167 (K17327), ab71+c168 (K17328), ab71+c169 (K17329), ab71+c170 (K17330), ab71+c171 (K17331), ab71+c172 (K17332), ab71+c173 (K17333), ab71+c174 (K17334), ab71+c175 (K17335), ab71+c176 (K17336), ab71+c177 (K17337), ab71+c178 (K17338), ab71+c179 (K17339), ab71+c180 (K17340), ab71+c181 (K17341), ab71+c182 (K17342), ab71+c183 (K17343), ab71+c184 (K17344), ab71+c185 (K17345), ab71+c186 (K17346), ab71+c187 (K17347), ab71+c188 (K17348), ab71+c189 (K17349), ab71+c190 (K17350), ab71+c191 (K17351), ab71+c192 (K17352), ab71+c193 (K17353), ab71+c194 (K17354), ab71+c195 (K17355), ab71+c196 (K17356), ab71+c197 (K17357), ab71+c198 (K17358), ab71+c199 (K17359), ab71+c200 (K17360), ab1+c201 (K17361), ab1+c202 (K17362), ab1+c203 (K17363), ab1+c204 (K17364), ab1+c205 (K17365), ab1+c206 (K17366), ab1+c207 (K17367), ab1+c208 (K17368), ab1+c209 (K17369), ab1+c210 (K17370), ab1+c211 (K17371), ab2+c201 (K17372), ab2+c202 (K17373), ab2+c203 (K17374), ab2+c204 (K17375), ab2+c205 (K17376), ab2+c206 (K17377), ab2+c207 (K17378), ab2+c208 (K17379), ab2+c209 (K17380), ab2+c210 (K17381), ab2+c211 (K17382), ab3+c201 (K17383), ab3+c202 (K17384), ab3+c203 (K17385), ab3+c204 (K17386), ab3+c205 (K17387), ab3+c206 (K17388), ab3+c207 (K17389), ab3+c208 (K17390), ab3+c209 (K17391), ab3+c210 (K17392), ab3+c211 (K17393), ab4+c201 (K17394), ab4+c202 (K17395), ab4+c203 (K17396), ab4+c204 (K17397), ab4+c205 (K17398), ab4+c206 (K17399), ab4+c207 (K17400), ab4+c208 (K17401), ab4+c209 (K17402), ab4+c210 (K17403), ab4+c211 (K17404), ab5+c201 (K17405), ab5+c202 (K17406), ab5+c203 (K17407), ab5+c204 (K17408), ab5+c205 (K17409), ab5+c206 (K17410), ab5+c207 (K17411), ab5+c208 (K17412), ab5+c209 (K17413), ab5+c210 (K17414), ab5+c211 (K17415), ab6+c201 (K17416), ab6+c202 (K17417), ab6+c203 (K17418), ab6+c204 (K17419), ab6+c205 (K17420), ab6+c206 (K17421), ab6+c207 (K17422), ab6+c208 (K17423), ab6+c209 (K17424), ab6+c210 (K17425), ab6+c211 (K17426), ab7+c201 (K17427), ab7+c202 (K17428), ab7+c203 (K17429), ab7+c204 (K17430), ab7+c205 (K17431), ab7+c206 (K17432), ab7+c207 (K17433), ab7+c208 (K17434), ab7+c209 (K17435), ab7+c210 (K17436), ab7+c211 (K17437), ab8+c201 (K17438), ab8+c202 (K17439), ab8+c203 (K17440), ab8+c204 (K17441), ab8+c205 (K17442), ab8+c206 (K17443), ab8+c207 (K17444), ab8+c208 (K17445), ab8+c209 (K17446), ab8+c210 (K17447), ab8+c211 (K17448), ab9+c201 (K17449), ab9+c202 (K17450), ab9+c203 (K17451), ab9+c204 (K17452), ab9+c205 (K17453), ab9+c206 (K17454), ab9+c207 (K17455), ab9+c208 (K17456), ab9+c209 (K17457), ab9+c210 (K17458), ab9+c211 (K17459), ab10+c201 (K17460), ab10+c202 (K17461), ab10+c203 (K17462), ab10+c204 (K17463), ab10+c205 (K17464), ab10+c206 (K17465), ab10+c207 (K17466), ab10+c208 (K17467), ab10+c209 (K17468), ab10+c210 (K17469), ab10+c211 (K17470), ab11+c201 (K17471), ab11+c202 (K17472), ab11+c203 (K17473), ab11+c204 (K17474), ab11+c205 (K17475), ab11+c206 (K17476), ab11+c207 (K17477), ab11+c208 (K17478), ab11+c209 (K17479), ab11+c210 (K17480), ab11+c211 (K17481), ab12+c201 (K17482), ab12+c202 (K17483), ab12+c203 (K17484), ab12+c204 (K17485), ab12+c205 (K17486), ab12+c206 (K17487), ab12+c207 (K17488), ab12+c208 (K17489), ab12+c209 (K17490), ab12+c210 (K17491), ab12+c211 (K17492), ab13+c201 (K17493), ab13+c202 (K17494), ab13+c203 (K17495), ab13+c204 (K17496), ab13+c205 (K17497), ab13+c206 (K17498), ab13+c207 (K17499), ab13+c208 (K17500), ab13+c209 (K17501), ab13+c210 (K17502), ab13+c211 (K17503), ab14+c201 (K17504), ab14+c202 (K17505), ab14+c203 (K17506), ab14+c204 (K17507), ab14+c205 (K17508), ab14+c206 (K17509), ab14+c207 (K17510), ab14+c208 (K17511), ab14+c209 (K17512), ab14+c210 (K17513), ab14+c211 (K17514), ab15+c201 (K17515), ab15+c202 (K17516), ab15+c203 (K17517), ab15+c204 (K17518) ab15+c205 (K17519), ab15+c206 (K17520), ab15+c207 (K17521), ab15+c208 (K17522), ab15+c209 (K17523), ab15+c210 (K17524), ab15+c211 (K17525), ab16+c201 (K17526), ab16+c202 (K17527), ab16+c203 (K17528), ab16+c204 (K17529), ab16+c205 (K17530), ab16+c206 (K17531), ab16+c207 (K17532), ab16+c208 (K17533), ab16+c209 (K17534), ab16+c210 (K17535), ab16+c211 (K17536), ab17+c201 (K17537), ab17+c202 (K17538), ab17+c203 (K17539), ab17+c204 (K17540), ab17+c205 (K17541), ab17+c206 (K17542), ab17+c207 (K17543), ab17+c208 (K17544), ab17+c209 (K17545), ab17+c210 (K17546), ab17+c211 (K17547), ab18+c201 (K17548), ab18+c202 (K17549), ab18+c203 (K17550), ab18+c204 (K17551), ab18+c205 (K17552), ab18+c206 (K17553), ab18+c207 (K17554), ab18+c208 (K17555), ab18+c209 (K17556), ab18+c210 (K17557), ab18+c211 (K17558), ab19+c201 (K17559), ab19+c202 (K17560), ab19+c203 (K17561), ab19+c204 (K17562), ab19+c205 (K17563), ab19+c206 (K17564), ab19+c207 (K17565), ab19+c208 (K17566), ab19+c209 (K17567), ab19+c210 (K17568), ab19+c211 (K17569), ab20+c201 (K17570), ab20+c202 (K17571), ab20+c203 (K17572), ab20+c204 (K17573), ab20+c205 (K17574), ab20+c206 (K17575), ab20+c207 (K17576), ab20+c208 (K17577), ab20+c209 (K17578), ab20+c210 (K17579), ab20+c211 (K17580), ab21+c201 (K17581), ab21+c202 (K17582), ab21+c203 (K17583), ab21+c204 (K17584), ab21+c205 (K17585), ab21+c206 (K17586), ab21+c207 (K17587), ab21+c208 (K17588), ab21+c209 (K17589), ab21+c210 (K17590), ab21+c211 (K17591), ab22+c201 (K17592), ab22+c202 (K17593) ab22+c203 (K17594), ab22+c204 (K17595), ab22+c205 (K17596), ab22+c206 (K17597), ab22+c207 (K17598), ab22+c208 (K17599), ab22+c209 (K17600), ab22+c210 (K17601), ab22+c211 (K17602), ab23+c201 (K17603), ab23+c202 (K17604), ab23+c203 (K17605), ab23+c204 (K17606), ab23+c205 (K17607), ab23+c206 (K17608), ab23+c207 (K17609), ab23+c208 (K17610), ab23+c209 (K17611), ab23+c210 (K17612), ab23+c211 (K17613), ab24+c201 (K17614), ab24+c202 (K17615), ab24+c203 (K17616), ab24+c204 (K17617), ab24+c205 (K17618), ab24+c206 (K17619), ab24+c207 (K17620), ab24+c208 (K17621), ab24+c209 (K17622), ab24+c210 (K17623), ab24+c211 (K17624), ab25+c201 (K17625), ab25+c202 (K17626), ab25+c203 (K17627), ab25+c204 (K17628), ab25+c205 (K17629), ab25+c206 (K17630), ab25+c207 (K17631), ab25+c208 (K17632), ab25+c209 (K17633), ab25+c210 (K17634), ab25+c211 (K17635), ab26+c201 (K17636), ab26+c202 (K17637), ab26+c203 (K17638), ab26+c204 (K17639), ab26+c205 (K17640), ab26+c206 (K17641), ab26+c207 (K17642), ab26+c208 (K17643), ab26+c209 (K17644), ab26+c210 (K17645), ab26+c211 (K17646), ab27+c201 (K17647), ab27+c202 (K17648), ab27+c203 (K17649), ab27+c204 (K17650), ab27+c205 (K17651), ab27+c206 (K17652), ab27+c207 (K17653), ab27+c208 (K17654), ab27+c209 (K17655), ab27+c210 (K17656), ab27+c211 (K17657), ab28+c201 (K17658), ab28+c202 (K17659), ab28+c203 (K17660), ab28+c204 (K17661), ab28+c205 (K17662), ab28+c206 (K17663), ab28+c207 (K17664), ab28+c208 (K17665), ab28+c209 (K17666), ab28+c210 (K17667), ab28+c211 (K17668), ab29+c201 (K17669), ab29+c202 (K17670), ab29+c203 (K17671), ab29+c204 (K17672), ab29+c205 (K17673), ab29+c206 (K17674), ab29+c207 (K17675), ab29+c208 (K17676), ab29+c209 (K17677), ab29+c210 (K17678), ab29+c211 (K17679), ab30+c201 (K17680), ab30+c202 (K17681), ab30+c203 (K17682), ab30+c204 (K17683), ab30+c205 (K17684), ab30+c206 (K17685), ab30+c207 (K17686), ab30+c208 (K17687), ab30+c209 (K17688), ab30+c210 (K17689), ab30+c211 (K17690), ab31+c201 (K17691), ab31+c202 (K17692), ab31+c203 (K17693), ab31+c204 (K17694), ab31+c205 (K17695), ab31+c206 (K17696), ab31+c207 (K17697), ab31+c208 (K17698), ab31+c209 (K17699), ab31+c210 (K17700), ab31+c211 (K17701), ab32+c201 (K17702), ab32+c202 (K17703), ab32+c203 (K17704), ab32+c204 (K17705), ab32+c205 (K17706), ab32+c206 (K17707), ab32+c207 (K17708), ab32+c208 (K17709), ab32+c209 (K17710), ab32+c210 (K17711), ab32+c211 (K17712), ab33+c201 (K17713), ab33+c202 (K17714), ab33+c203 (K17715), ab33+c204 (K17716), ab33+c205 (K17717), ab33+c206 (K17718), ab33+c207 (K17719), ab33+c208 (K17720), ab33+c209 (K17721), ab33+c210 (K17722), ab33+c211 (K17723), ab34+c201 (K17724), ab34+c202 (K17725), ab34+c203 (K17726), ab34+c204 (K17727), ab34+c205 (K17728), ab34+c206 (K17729), ab34+c207 (K17730), ab34+c208 (K17731), ab34+c209 (K17732), ab34+c210 (K17733), ab34+c211 (K17734), ab35+c201 (K17735), ab35+c202 (K17736), ab35+c203 (K17737), ab35+c204 (K17738), ab35+c205 (K17739), ab35+c206 (K17740), ab35+c207 (K17741), ab35+c208 (K17742), ab35+c209 (K17743), ab35+c210 (K17744), ab35+c211 (K17745), ab36+c201 (K17746), ab36+c202 (K17747), ab36+c203 (K17748), ab36+c204 (K17749), ab36+c205 (K17750), ab36+c206 (K17751), ab36+c207 (K17752), ab36+c208 (K17753), ab36+c209 (K17754), ab36+c210 (K17755), ab36+c211 (K17756), ab37+c201 (K17757), ab37+c202 (K17758), ab37+c203 (K17759), ab37+c204 (K17760), ab37+c205 (K17761), ab37+c206 (K17762), ab37+c207 (K17763), ab37+c208 (K17764), ab37+c209 (K17765), ab37+c210 (K17766), ab37+c211 (K17767), ab38+c201 (K17768), ab38+c202 (K17769), ab38+c203 (K17770), ab38+c204 (K17771), ab38+c205 (K17772), ab38+c206 (K17773), ab38+c207 (K17774), ab38+c208 (K17775), ab38+c209 (K17776), ab38+c210 (K17777), ab38+c211 (K17778), ab39+c201 (K17779), ab39+c202 (K17780), ab39+c203 (K17781), ab39+c204 (K17782), ab39+c205 (K17783), ab39+c206 (K17784), ab39+c207 (K17785), ab39+c208 (K17786), ab39+c209 (K17787), ab39+c210 (K17788), ab39+c211 (K17789), ab40+c201 (K17790), ab40+c202 (K17791), ab40+c203 (K17792), ab40+c204 (K17793), ab40+c205 (K17794), ab40+c206 (K17795), ab40+c207 (K17796), ab40+c208 (K17797), ab40+c209 (K17798), ab40+c210 (K17799), ab40+c211 (K17800), ab41+c201 (K17801), ab41+c202 (K17802), ab41+c203 (K17803), ab41+c204 (K17804), ab41+c205 (K17805), ab41+c206 (K17806), ab41+c207 (K17807), ab41+c208 (K17808), ab41+c209 (K17809), ab41+c210 (K17810), ab41+c211 (K17811), ab42+c201 (K17812), ab42+c202 (K17813), ab42+c203 (K17814), ab42+c204 (K17815), ab42+c205 (K17816), ab42+c206 (K17817), ab42+c207 (K17818), ab42+c208 (K17819), ab42+c209 (K17820), ab42+c210 (K17821), ab42+c211 (K17822), ab43+c201 (K17823), ab43+c202 (K17824), ab43+c203 (K17825), ab43+c204 (K17826), ab43+c205 (K17827), ab43+c206 (K17828), ab43+c207 (K17829), ab43+c208 (K17830), ab43+c209 (K17831), ab43+c210 (K17832), ab43+c211 (K17833), ab44+c201 (K17834), ab44+c202 (K17835), ab44+c203 (K17836), ab44+c204 (K17837), ab44+c205 (K17838), ab44+c206 (K17839), ab44+c207 (K17840), ab44+c208 (K17841), ab44+c209 (K17842), ab44+c210 (K17843), ab44+c211 (K17844), ab45+c201 (K17845), ab45+c202 (K17846), ab45+c203 (K17847), ab45+c204 (K17848), ab45+c205 (K17849), ab45+c206 (K17850), ab45+c207 (K17851), ab45+c208 (K17852), ab45+c209 (K17853), ab45+c210 (K17854), ab45+c211 (K17855), ab46+c201 (K17856), ab46+c202 (K17857), ab46+c203 (K17858), ab46+c204 (K17859), ab46+c205 (K17860), ab46+c206 (K17861), ab46+c207 (K17862), ab46+c208 (K17863), ab46+c209 (K17864), ab46+c210 (K17865), ab46+c211 (K17866), ab47+c201 (K17867), ab47+c202 (K17868), ab47+c203 (K17869), ab47+c204 (K17870), ab47+c205 (K17871), ab47+c206 (K17872), ab47+c207 (K17873), ab47+c208 (K17874), ab47+c209 (K17875), ab47+c210 (K17876), ab47+c211 (K17877), ab48+c201 (K17878), ab48+c202 (K17879), ab48+c203 (K17880), ab48+c204 (K17881), ab48+c205 (K17882), ab48+c206 (K17883), ab48+c207 (K17884), ab48+c208 (K17885), ab48+c209 (K17886), ab48+c210 (K17887), ab48+c211 (K17888), ab49+c201 (K17889), ab49+c202 (K17890), ab49+c203 (K17891), ab49+c204 (K17892), ab49+c205 (K17893), ab49+c206 (K17894), ab49+c207 (K17895), ab49+c208 (K17896), ab49+c209 (K17897), ab49+c210 (K17898), ab49+c211 (K17899), ab50+c201 (K17900), ab50+c202 (K17901), ab50+c203 (K17902), ab50+c204 (K17903), ab50+c205 (K17904), ab50+c206 (K17905), ab50+c207 (K17906), ab50+c208 (K17907), ab50+c209 (K17908), ab50+c210 (K17909), ab50+c211 (K17910), ab51+c201 (K17911), ab51+c202 (K17912), ab51+c203 (K17913), ab51+c204 (K17914), ab51+c205 (K17915), ab51+c206 (K17916), ab51+c207 (K17917), ab51+c208 (K17918), ab51+c209 (K17919), ab51+c210 (K17920), ab51+c211 (K17921), ab52+c201 (K17922), ab52+c202 (K17923), ab52+c203 (K17924), ab52+c204 (K17925), ab52+c205 (K17926), ab52+c206 (K17927), ab52+c207 (K17928), ab52+c208 (K17929), ab52+c209 (K17930), ab52+c210 (K17931), ab52+c211 (K17932), ab53+c201 (K17933), ab53+c202 (K17934), ab53+c203 (K17359), ab53+c204 (K17936), ab53+c205 (K17937), ab53+c206 (K17938), ab53+c207 (K17939), ab53+c208 (K17940), ab53+c209 (K17941), ab53+c210 (K17942), ab53+c211 (K17943), ab54+c201 (K17944), ab54+c202 (K17945), ab54+c203 (K17946), ab54+c204 (K17947), ab54+c205 (K17948), ab54+c206 (K17949), ab54+c207 (K17950), ab54+c208 (K17951), ab54+c209 (K17952), ab54+c210 (K17953), ab54+c211 (K17954), ab55+c201 (K17955), ab55+c202 (K17956), ab55+c203 (K17957), ab55+c204 (K17958), ab55+c205 (K17959), ab55+c206 (K17960), ab55+c207 (K17961), ab55+c208 (K17962), ab55+c209 (K17963), ab55+c210 (K17964), ab55+c211 (K17965), ab56+c201 (K17966), ab56+c202 (K17967), ab56+c203 (K17968), ab56+c204 (K17969), ab56+c205 (K17970), ab56+c206 (K17971), ab56+c207 (K17972), ab56+c208 (K17973), ab56+c209 (K17974), ab56+c210 (K17975), ab56+c211 (K17976), ab57+c201 (K17977), ab57+c202 (K17978), ab57+c203 (K17979), ab57+c204 (K17980), ab57+c205 (K17981), ab57+c206 (K17982), ab57+c207 (K17983), ab57+c208 (K17984), ab57+c209 (K17985), ab57+c210 (K17986), ab57+c211 (K17987), ab58+c201 (K17988), ab58+c202 (K17989), ab58+c203 (K17990), ab58+c204 (K17991), ab58+c205 (K17992), ab58+c206 (K17993), ab58+c207 (K17994), ab58+c208 (K17995), ab58+c209 (K17996), ab58+c210 (K17997), ab58+c211 (K17998), ab59+c201 (K17999), ab59+c202 (K18000), ab59+c203 (K18001), ab59+c204 (K18002), ab59+c205 (K18003), ab59+c206 (K18004), ab59+c207 (K18005), ab59+c208 (K18006), ab59+c209 (K18007), ab59+c210 (K18008), ab59+c211 (K18009), ab60+c201 (K18010), ab60+c202 (K18011), ab60+c203 (K18012), ab60+c204 (K18013), ab60+c205 (K18014), ab60+c206 (K18015), ab60+c207 (K18016), ab60+c208 (K18017), ab60+c209 (K18018), ab60+c210 (K18019), ab60+c211 (K18020), ab61+c201 (K18021), ab61+c202 (K18022), ab61+c203 (K18023), ab61+c204 (K18024), ab61+c205 (K18025), ab61+c206 (K18026), ab61+c207 (K18027), ab61+c208 (K18028), ab61+c209 (K18029), ab61+c210 (K18030), ab61+c211 (K18031), ab62+c201 (K18032), ab62+c202 (K18033), ab62+c203 (K18034), ab62+c204 (K18035), ab62+c205 (K18036), ab62+c206 (K18037), ab62+c207 (K18038), ab62+c208 (K18039), ab62+c209 (K18040), ab62+c210 (K18041), ab62+c211 (K18042), ab63+c201 (K18043), ab63+c202 (K18044), ab63+c203 (K18045), ab63+c204 (K18046), ab63+c205 (K18047), ab63+c206 (K18048), ab63+c207 (K18049), ab63+c208 (K18050), ab63+c209 (K18051), ab63+c210 (K18052), ab63+c211 (K18053), ab64+c201 (K18054), ab64+c202 (K18055), ab64+c203 (K18056), ab64+c204 (K18057), ab64+c205 (K18058), ab64+c206 (K18059), ab64+c207 (K18060), ab64+c208 (K18061), ab64+c209 (K18062), ab64+c210 (K18063), ab64+c211 (K18064), ab65+c201 (K18065), ab65+c202 (K18066), ab65+c203 (K18067), ab65+c204 (K18068), ab65+c205 (K18069), ab65+c206 (K18070), ab65+c207 (K18071), ab65+c208 (K18072), ab65+c209 (K18073), ab65+c210 (K18074), ab65+c211 (K18075), ab66+c201 (K18076), ab66+c202 (K18077), ab66+c203 (K18078), ab66+c204 (K18079), ab66+c205 (K18080), ab66+c206 (K18081), ab66+c207 (K18082), ab66+c208 (K18083), ab66+c209 (K18084), ab66+c210 (K18085), ab66+c211 (K18086), ab67+c201 (K18087), ab67+c202 (K18088), ab67+c203 (K18089), ab67+c204 (K18090), ab67+c205 (K18091), ab67+c206 (K18092), ab67+c207 (K18093), ab67+c208 (K18094), ab67+c209 (K18095), ab67+c210 (K18096), ab67+c211 (K18097), ab68+c201 (K18098), ab68+c202 (K18099), ab68+c203 (K18100), ab68+c204 (K18101), ab68+c205 (K18102), ab68+c206 (K18103), ab68+c207 (K18104), ab68+c208 (K18105), ab68+c209 (K18106), ab68+c210 (K18107), ab68+c211 (K18108), ab69+c201 (K18109), ab69+c202 (K18110), ab69+c203 (K18111), ab69+c204 (K18112), ab69+c205 (K18113), ab69+c206 (K18114), ab69+c207 (K18115), ab69+c208 (K18116), ab69+c209 (K18117), ab69+c210 (K18118), ab69+c211 (K18119), ab70+c201 (K18120), ab70+c202 (K18121), ab70+c203 (K18122), ab70+c204 (K18123), ab70+c205 (K18124), ab70+c206 (K18125), ab70+c207 (K18126), ab70+c208 (K18127), ab70+c209 (K18128), ab70+c210 (K18129), ab70+c211 (K18130), ab71+c201 (K18131), ab71+c202 (K18132), ab71+c203 (K18133), ab71+c204 (K18134), ab71+c205 (K18135), ab71+c206 (K18136), ab71+c207 (K18137), ab71+c208 (K18138), ab71+c209 (K18139), ab71+c210 (K18140), ab71+c211 (K18141), ab72+c1 (K19766), ab72+c2 (K19767), ab72+c3 (K19768), ab72+c4 (K19769), ab72+c5 (K19770), ab72+c6 (K19771), ab72+c7 (K19772), ab72+c8 (K19773), ab72+c9 (K19774), ab72+c10 (K19775), ab72+c11 (K19776), ab72+c12 (K19777), ab72+c13 (K19778), ab72+c14 (K19779), ab72+c15 (K19780), ab72+c16 (K19781), ab72+c17 (K19782), ab72+c18 (K19783), ab72+c19 (K19784), ab72+c20 (K19785), ab72+c21 (K19786), ab72+c22 (K19787), ab72+c23 (K19788), ab72+c24 (K19789), ab72+c25 (K19790), ab72+c26 (K19791), ab72+c27 (K19792), ab72+c28 (K19793), ab72+c29 (K19794), ab72+c30 (K19795), ab72+c31 (K19796), ab72+c32 (K19797), ab72+c33 (K19798), ab72+c34 (K19799), ab72+c35 (K19800), ab72+c36 (K19801), ab72+c37 (K19802), ab72+c38 (K19803), ab72+c39 (K19804), ab72+c40 (K19805), ab72+c41 (K19806), ab72+c42 (K19807), ab72+c43 (K19808), ab72+c44 (K19809), ab72+c45 (K19810), ab72+c46 (K19811), ab72+c47 (K19812), ab72+c48 (K19813), ab72+c49 (K19814), ab72+c50 (K19815), ab72+c51 (K19816), ab72+c52 (K19817), ab72+c53 (K19818), ab72+c54 (K19819), ab72+c55 (K19820), ab72+c56 (K19821), ab72+c57 (K19822), ab72+c58 (K19823), ab72+c59 (K19824), ab72+c60 (K19825), ab72+c61 (K19826), ab72+c62 (K19827), ab72+c63 (K19828), ab72+c64 (K19829), ab72+c65 (K19830), ab72+c66 (K19831), ab72+c67 (K19832), ab72+c68 (K19833), ab72+c69 (K19834), ab72+c70 (K19835), ab72+c71 (K19836), ab72+c72 (K19837), ab72+c73 (K19838), ab72+c74 (K19839), ab72+c75 (K19840), ab72+c76 (K19841), ab72+c77 (K19842), ab72+c78 (K19843), ab72+c79 (K19844), ab72+c80 (K19845), ab72+c81 (K19846), ab72+c82 (K19847), ab72+c83 (K19848), ab72+c84 (K19849), ab72+c85 (K19850), ab72+c86 (K19851), ab72+c87 (K19852), ab72+c88 (K19853), ab72+c89 (K19854), ab72+c90 (K19855), ab72+c91 (K19856), ab72+c92 (K19857), ab72+c93 (K19858), ab72+c94 (K19859), ab72+c95 (K19860), ab72+c96 (K19861), ab72+c97 (K19862), ab72+c98 (K19863), ab72+c99 (K19864), ab72+c100 (K19865), ab72+c101 (K19866), ab72+c102 (K19867), ab72+c103 (K19868), ab72+c104 (K19869), ab72+c105 (K19870), ab72+c106 (K19871), ab72+c107 (K19872), ab72+c108 (K19873), ab72+c109 (K19874), ab72+c110 (K19875), ab72+c111 (K19876), ab72+c112 (K19877), ab72+c113 (K19878), ab72+c114 (K19879), ab72+c115 (K19880), ab72+c116 (K19881), ab72+c117 (K19882), ab72+c118 (K19883), ab72+c119 (K19884), ab72+c120 (K19885), ab72+c121 (K19886), ab72+c122 (K19887), ab72+c123 (K19888), ab72+c124 (K19889), ab72+c125 (K19890), ab72+c126 (K19891), ab72+c127 (K19892), ab72+c128 (K19893), ab72+c129 (K19894), ab72+c130 (K19895), ab72+c131 (K19896), ab72+c132 (K19897), ab72+c133 (K19898), ab72+c134 (K19899), ab72+c135 (K19900), ab72+c136 (K19901), ab72+c137 (K19902), ab72+c138 (K19903), ab72+c139 (K19904), ab72+c140 (K19905), ab72+c141 (K19906), ab72+c142 (K19907), ab72+c143 (K19908), ab72+c144 (K19909), ab72+c145 (K19910), ab72+c146 (K19911), ab72+c147 (K19912), ab72+c148 (K19913), ab72+c149 (K19914), ab72+c150 (K19915), ab72+c151 (K19916), ab72+c152 (K19917), ab72+c153 (K19918), ab72+c154 (K19919), ab72+c155 (K19920), ab72+c156 (K19921), ab72+c157 (K19922), ab72+c158 (K19923), ab72+c159 (K19924), ab72+c160 (K19925), ab72+c161 (K19926), ab72+c162 (K19927), ab72+c163 (K19928), ab72+c164 (K19929), ab72+c165 (K19930), ab72+c166 (K19931), ab72+c167 (K19932), ab72+c168 (K19933), ab72+c169 (K19934), ab72+c170 (K19935), ab72+c171 (K19936), ab72+c172 (K19937), ab72+c173 (K19938), ab72+c174 (K19939), ab72+c175 (K19940), ab72+c176 (K19941), ab72+c177 (K19942), ab72+c178 (K19943), ab72+c179 (K19944), ab72+c180 (K19945), ab72+c181 (K19946), ab72+c182 (K19947), ab72+c183 (K19948), ab72+c184 (K19949), ab72+c185 (K19950), ab72+c186 (K19951), ab72+c187 (K19952), ab72+c188 (K19953), ab72+c189 (K19954), ab72+c190 (K19955), ab72+c191 (K19956), ab72+c192 (K19957), ab72+c193 (K19958), ab72+c194 (K19959), ab72+c195 (K19960), ab72+c196 (K19961), ab72+c197 (K19962), ab72+c198 (K19963), ab72+c199 (K19964), ab72+c200 (K19965), ab72+c201 (K19966), ab72+c202 (K19967), ab72+c203 (K19968), ab72+c204 (K19969), ab72+c205 (K19970), ab72+c206 (K19971), ab72+c207 (K19972), ab72+c208 (K19973), ab72+c209 (K19974), ab72+c210 (K19975), ab72+c211 (K19976), ab73+c1 (K19977), ab73+c2 (K19978), ab73+c3 (K19979), ab73+c4 (K19980), ab73+c5 (K19981), ab73+c6 (K19982), ab73+c7 (K19983), ab73+c8 (K19984), ab73+c9 (K19985), ab73+c10 (K19986), ab73+c11 (K19987), ab73+c12 (K19988), ab73+c13 (K19989), ab73+c14 (K19990), ab73+c15 (K19991), ab73+c16 (K19992), ab73+c17 (K19993), ab73+c18 (K19994), ab73+c19 (K19995), ab73+c20 (K19996), ab73+c21 (K19997), ab73+c22 (K19998), ab73+c23 (K19999), ab73+c24 (K20000), ab73+c25 (K20001), ab73+c26 (K20002), ab73+c27 (K20003), ab73+c28 (K20004), ab73+c29 (K20005), ab73+c30 (K20006), ab73+c31 (K20007), ab73+c32 (K20008), ab73+c33 (K20009), ab73+c34 (K20010), ab73+c35 (K20011), ab73+c36 (K20012), ab73+c37 (K20013), ab73+c38 (K20014), ab73+c39 (K20015), ab73+c40 (K20016), ab73+c41 (K20017), ab73+c42 (K20018), ab73+c43 (K20019), ab73+c44 (K20020), ab73+c45 (K20021), ab73+c46 (K20022), ab73+c47 (K20023), ab73+c48 (K20024), ab73+c49 (K20025), ab73+c50 (K20026), ab73+c51 (K20027), ab73+c52 (K20028), ab73+c53 (K20029), ab73+c54 (K20030), ab73+c55 (K20031), ab73+c56 (K20032), ab73+c57 (K20033), ab73+c58 (K20034), ab73+c59 (K20035), ab73+c60 (K20036), ab73+c61 (K20037), ab73+c62 (K20038), ab73+c63 (K20039), ab73+c64 (K20040), ab73+c65 (K20041), ab73+c66 (K20042), ab73+c67 (K20043), ab73+c68 (K20044), ab73+c69 (K20045), ab73+c70 (K20046), ab73+c71 (K20047), ab73+c72 (K20048), ab73+c73 (K20049), ab73+c74 (K20050), ab73+c75 (K20051), ab73+c76 (K20052), ab73+c77 (K20053), ab73+c78 (K20054), ab73+c79 (K20055), ab73+c80 (K20056), ab73+c81 (K20057), ab73+c82 (K20058), ab73+c83 (K20059), ab73+c84 (K20060), ab73+c85 (K20061), ab73+c86 (K20062), ab73+c87 (K20063), ab73+c88 (K20064), ab73+c89 (K20065), ab73+c90 (K20066), ab73+c91 (K20067), ab73+c92 (K20068), ab73+c93 (K20069), ab73+c94 (K20070), ab73+c95 (K20071), ab73+c96 (K20072), ab73+c97 (K20073), ab73+c98 (K20074), ab73+c99 (K20075), ab73+c100 (K20076), ab73+c101 (K20077), ab73+c102 (K20078), ab73+c103 (K20079), ab73+c104 (K20080), ab73+c105 (K20081), ab73+c106 (K20082), ab73+c107 (K20083), ab73+c108 (K20084), ab73+c109 (K20085), ab73+c110 (K20086), ab73+c111 (K20087), ab73+c112 (K20088), ab73+c113 (K20089), ab73+c114 (K20090), ab73+c115 (K20091), ab73+c116 (K20092), ab73+c117 (K20093), ab73+c118 (K20094), ab73+c119 (K20095), ab73+c120 (K20096), ab73+c121 (K20097), ab73+c122 (K20098), ab73+c123 (K20099), ab73+c124 (K20100), ab73+c125 (K20101), ab73+c126 (K20102), ab73+c127 (K20103), ab73+c128 (K20104), ab73+c129 (K20105), ab73+c130 (K20106), ab73+c131 (K20107), ab73+c132 (K20108), ab73+c133 (K20109), ab73+c134 (K20110), ab73+c135 (K20111), ab73+c136 (K20112), ab73+c137 (K20113), ab73+c138 (K20114), ab73+c139 (K20115), ab73+c140 (K20116), ab73+c141 (K20117), ab73+c142 (K20118), ab73+c143 (K20119), ab73+c144 (K20120), ab73+c145 (K20121), ab73+c146 (K20122), ab73+c147 (K20123), ab73+c148 (K20124), ab73+c149 (K20125), ab73+c150 (K20126), ab73+c151 (K20127), ab73+c152 (K20128), ab73+c153 (K20129), ab73+c154 (K20130), ab73+c155 (K20131), ab73+c156 (K20132), ab73+c157 (K20133), ab73+c158 (K20134), ab73+c159 (K20135), ab73+c160 (K20136), ab73+c161 (K20137), ab73+c162 (K20138), ab73+c163 (K20139), ab73+c164 (K20140), ab73+c165 (K20141), ab73+c166 (K20142), ab73+c167 (K20143), ab73+c168 (K20144), ab73+c169 (K20145), ab73+c170 (K20146), ab73+c171 (K20147), ab73+c172 (K20148), ab73+c173 (K20149), ab73+c174 (K20150), ab73+c175 (K20151), ab73+c176 (K20152), ab73+c177 (K20153), ab73+c178 (K20154), ab73+c179 (K20155), ab73+c180 (K20156), ab73+c181 (K20157), ab73+c182 (K20158), ab73+c183 (K20159), ab73+c184 (K20160), ab73+c185 (K20161), ab73+c186 (K20162), ab73+c187 (K20163), ab73+c188 (K20164), ab73+c189 (K20165), ab73+c190 (K20166), ab73+c191 (K20167), ab73+c192 (K20168), ab73+c193 (K20169), ab73+c194 (K20170), ab73+c195 (K20171), ab73+c196 (K20172), ab73+c197 (K20173), ab73+c198 (K20174), ab73+c199 (K20175), ab73+c200 (K20176), ab73+c201 (K20177), ab73+c202 (K20178), ab73+c203 (K20179), ab73+c204 (K20180), ab73+c205 (K20181), ab73+c206 (K20182), ab73+c207 (K20183), ab73+c208 (K20184), ab73+c209 (K20185), ab73+c210 (K20186), ab73+c211 (K20187), ab74+c1 (K20188), ab74+c2 (K20189), ab74+c3 (K20190), ab74+c4 (K20191), ab74+c5 (K20192), ab74+c6 (K20193), ab74+c7 (K20194), ab74+c8 (K20195), ab74+c9 (K20196), ab74+c10 (K20197), ab74+c11 (K20198), ab74+c12 (K20199), ab74+c13 (K20200), ab74+c14 (K20201), ab74+c15 (K20202), ab74+c16 (K20203), ab74+c17 (K20204), ab74+c18 (K20205), ab74+c19 (K20206), ab74+c20 (K20207), ab74+c21 (K20208), ab74+c22 (K20209), ab74+c23 (K20210), ab74+c24 (K20211), ab74+c25 (K20212), ab74+c26 (K20213), ab74+c27 (K20214), ab74+c28 (K20215), ab74+c29 (K20216), ab74+c30 (K20217), ab74+c31 (K20218), ab74+c32 (K20219), ab74+c33 (K20220), ab74+c34 (K20221), ab74+c35 (K20222), ab74+c36 (K20223), ab74+c37 (K20224), ab74+c38 (K20225), ab74+c39 (K20226), ab74+c40 (K20227), ab74+c41 (K20228), ab74+c42 (K20229), ab74+c43 (K20230), ab74+c44 (K20231), ab74+c45 (K20232), ab74+c46 (K20233), ab74+c47 (K20234), ab74+c48 (K20235), ab74+c49 (K20236), ab74+c50 (K20237), ab74+c51 (K20238), ab74+c52 (K20239), ab74+c53 (K20240), ab74+c54 (K20241), ab74+c55 (K20242), ab74+c56 (K20243), ab74+c57 (K20244), ab74+c58 (K20245), ab74+c59 (K20246), ab74+c60 (K20247), ab74+c61 (K20248), ab74+c62 (K20249), ab74+c63 (K20250), ab74+c64 (K20251), ab74+c65 (K20252), ab74+c66 (K20253), ab74+c67 (K20254), ab74+c68 (K20255), ab74+c69 (K20256), ab74+c70 (K20257), ab74+c71 (K20258), ab74+c72 (K20259), ab74+c73 (K20260), ab74+c74 (K20261), ab74+c75 (K20262), ab74+c76 (K20263), ab74+c77 (K20264), ab74+c78 (K20265), ab74+c79 (K20266), ab74+c80 (K20267), ab74+c81 (K20268), ab74+c82 (K20269), ab74+c83 (K20270), ab74+c84 (K20271), ab74+c85 (K20272), ab74+c86 (K20273), ab74+c87 (K20274), ab74+c88 (K20275), ab74+c89 (K20276), ab74+c90 (K20277), ab74+c91 (K20278), ab74+c92 (K20279), ab74+c93 (K20280), ab74+c94 (K20281), ab74+c95 (K20282), ab74+c96 (K20283), ab74+c97 (K20284), ab74+c98 (K20285), ab74+c99 (K20286), ab74+c100 (K20287), ab74+c101 (K20288), ab74+c102 (K20289), ab74+c103 (K20290), ab74+c104 (K20291), ab74+c105 (K20292), ab74+c106 (K20293), ab74+c107 (K20294), ab74+c108 (K20295), ab74+c109 (K20296), ab74+c110 (K20297), ab74+c111 (K20298), ab74+c112 (K20299), ab74+c113 (K20300), ab74+c114 (K20301), ab74+c115 (K20302), ab74+c116 (K20303), ab74+c117 (K20304), ab74+c118 (K20305), ab74+c119 (K20306), ab74+c120 (K20307), ab74+c121 (K20308), ab74+c122 (K20309), ab74+c123 (K20310), ab74+c124 (K20311), ab74+c125 (K20312), ab74+c126 (K20313), ab74+c127 (K20314), ab74+c128 (K20315), ab74+c129 (K20316), ab74+c130 (K20317), ab74+c131 (K20318), ab74+c132 (K20319), ab74+c133 (K20320), ab74+c134 (K20321), ab74+c135 (K20322), ab74+c136 (K20323), ab74+c137 (K20324), ab74+c138 (K20325), ab74+c139 (K20326), ab74+c140 (K20327), ab74+c141 (K20328), ab74+c142 (K20329), ab74+c143 (K20330), ab74+c144 (K20331), ab74+c145 (K20332), ab74+c146 (K20333), ab74+c147 (K20334), ab74+c148 (K20335), ab74+c149 (K20336), ab74+c150 (K20337), ab74+c151 (K20338), ab74+c152 (K20339), ab74+c153 (K20340), ab74+c154 (K20341), ab74+c155 (K20342), ab74+c156 (K20343), ab74+c157 (K20344), ab74+c158 (K20345), ab74+c159 (K20346), ab74+c160 (K20347), ab74+c161 (K20348), ab74+c162 (K20349), ab74+c163 (K20350), ab74+c164 (K20351), ab74+c165 (K20352), ab74+c166 (K20353), ab74+c167 (K20354), ab74+c168 (K20355), ab74+c169 (K20356), ab74+c170 (K20357), ab74+c171 (K20358), ab74+c172 (K20359), ab74+c173 (K20360), ab74+c174 (K20361), ab74+c175 (K20362), ab74+c176 (K20363), ab74+c177 (K20364), ab74+c178 (K20365), ab74+c179 (K20366), ab74+c180 (K20367), ab74+c181 (K20368), ab74+c182 (K20369), ab74+c183 (K20370), ab74+c184 (K20371), ab74+c185 (K20372), ab74+c186 (K20373), ab74+c187 (K20374), ab74+c188 (K20375), ab74+c189 (K20376), ab74+c190 (K20377), ab74+c191 (K20378), ab74+c192 (K20379), ab74+c193 (K20380), ab74+c194 (K20381), ab74+c195 (K20382), ab74+c196 (K20383), ab74+c197 (K20384), ab74+c198 (K20385), ab74+c199 (K20386), ab74+c200 (K20387), ab74+c201 (K20388), ab74+c202 (K20389), ab74+c203 (K20390), ab74+c204 (K20391), ab74+c205 (K20392), ab74+c206 (K20393), ab74+c207 (K20394), ab74+c208 (K20395), ab74+c209 (K20396), ab74+c210 (K20397), ab74+c211 (K20398), ab75+c1 (K20399), ab75+c2 (K20400), ab75+c3 (K20401), ab75+c4 (K20402), ab75+c5 (K20403), ab75+c6 (K20404), ab75+c7 (K20405), ab75+c8 (K20406), ab75+c9 (K20407), ab75+c10 (K20408), ab75+c11 (K20409), ab75+c12 (K20410), ab75+c13 (K20411), ab75+c14 (K20412), ab75+c15 (K20413), ab75+c16 (K20414), ab75+c17 (K20415), ab75+c18 (K20416), ab75+c19 (K20417), ab75+c20 (K20418), ab75+c21 (K20419), ab75+c22 (K20420), ab75+c23 (K20421), ab75+c24 (K20422), ab75+c25 (K20423), ab75+c26 (K20424), ab75+c27 (K20425), ab75+c28 (K20426), ab75+c29 (K20427), ab75+c30 (K20428), ab75+c31 (K20429), ab75+c32 (K20430), ab75+c33 (K20431), ab75+c34 (K20432), ab75+c35 (K20433), ab75+c36 (K20434), ab75+c37 (K20435), ab75+c38 (K20436), ab75+c39 (K20437), ab75+c40 (K20438), ab75+c41 (K20439), ab75+c42 (K20440), ab75+c43 (K20441), ab75+c44 (K20442), ab75+c45 (K20443), ab75+c46 (K20444), ab75+c47 (K20445), ab75+c48 (K20446), ab75+c49 (K20447), ab75+c50 (K20448), ab75+c51 (K20449), ab75+c52 (K20450), ab75+c53 (K20451), ab75+c54 (K20452), ab75+c55 (K20453), ab75+c56 (K20454), ab75+c57 (K20455), ab75+c58 (K20456), ab75+c59 (K20457), ab75+c60 (K20458), ab75+c61 (K20459), ab75+c62 (K20460), ab75+c63 (K20461), ab75+c64 (K20462), ab75+c65 (K20463), ab75+c66 (K20464), ab75+c67 (K20465), ab75+c68 (K20466), ab75+c69 (K20467), ab75+c70 (K20468), ab75+c71 (K20469), ab75+c72 (K20470), ab75+c73 (K20471), ab75+c74 (K20472), ab75+c75 (K20473), ab75+c76 (K20474), ab75+c77 (K20475), ab75+c78 (K20476), ab75+c79 (K20477), ab75+c80 (K20478), ab75+c81 (K20479), ab75+c82 (K20480), ab75+c83 (K20481), ab75+c84 (K20482), ab75+c85 (K20483), ab75+c86 (K20484), ab75+c87 (K20485), ab75+c88 (K20486), ab75+c89 (K20487), ab75+c90 (K20488), ab75+c91 (K20489), ab75+c92 (K20490), ab75+c93 (K20491), ab75+c94 (K20492), ab75+c95 (K20493), ab75+c96 (K20494), ab75+c97 (K20495), ab75+c98 (K20496), ab75+c99 (K20497), ab75+c100 (K20498), ab75+c101 (K20499), ab75+c102 (K20500), ab75+c103 (K20501), ab75+c104 (K20502), ab75+c105 (K20503), ab75+c106 (K20504), ab75+c107 (K20505), ab75+c108 (K20506), ab75+c109 (K20507), ab75+c110 (K20508), ab75+c111 (K20509), ab75+c112 (K20510), ab75+c113 (K20511), ab75+c114 (K20512), ab75+c115 (K20513), ab75+c116 (K20514), ab75+c117 (K20515), ab75+c118 (K20516), ab75+c119 (K20517), ab75+c120 (K20518), ab75+c121 (K20519), ab75+c122 (K20520), ab75+c123 (K20521), ab75+c124 (K20522), ab75+c125 (K20523), ab75+c126 (K20524), ab75+c127 (K20525), ab75+c128 (K20526), ab75+c129 (K20527), ab75+c130 (K20528), ab75+c131 (K20529), ab75+c132 (K20530), ab75+c133 (K20531), ab75+c134 (K20532), ab75+c135 (K20533), ab75+c136 (K20534), ab75+c137 (K20535), ab75+c138 (K20536), ab75+c139 (K20537), ab75+c140 (K20538), ab75+c141 (K20539), ab75+c142 (K20540), ab75+c143 (K20541), ab75+c144 (K20542), ab75+c145 (K20543), ab75+c146 (K20544), ab75+c147 (K20545), ab75+c148 (K20546), ab75+c149 (K20547), ab75+c150 (K20548), ab75+c151 (K20549), ab75+c152 (K20550), ab75+c153 (K20551), ab75+c154 (K20552), ab75+c155 (K20553), ab75+c156 (K20554), ab75+c157 (K20555), ab75+c158 (K20556), ab75+c159 (K20557), ab75+c160 (K20558), ab75+c161 (K20559), ab75+c162 (K20560), ab75+c163 (K20561), ab75+c164 (K20562), ab75+c165 (K20563), ab75+c166 (K20564), ab75+c167 (K20565), ab75+c168 (K20566), ab75+c169 (K20567), ab75+c170 (K20568), ab75+c171 (K20569), ab75+c172 (K20570), ab75+c173 (K20571), ab75+c174 (K20572), ab75+c175 (K20573), ab75+c176 (K20574), ab75+c177 (K20575), ab75+c178 (K20576), ab75+c179 (K20577), ab75+c180 (K20578), ab75+c181 (K20579), ab75+c182 (K20580), ab75+c183 (K20581), ab75+c184 (K20582), ab75+c185 (K20583), ab75+c186 (K20584), ab75+c187 (K20585), ab75+c188 (K20586), ab75+c189 (K20587), ab75+c190 (K20588), ab75+c191 (K20589), ab75+c192 (K20590), ab75+c193 (K20591), ab75+c194 (K20592), ab75+c195 (K20593), ab75+c196 (K20594), ab75+c197 (K20595), ab75+c198 (K20596), ab75+c199 (K20597), ab75+c200 (K20598), ab75+c201 (K20599), ab75+c202 (K20600), ab75+c203 (K20601), ab75+c204 (K20602), ab75+c205 (K20603), ab75+c206 (K20604), ab75+c207 (K20605), ab75+c208 (K20606), ab75+c209 (K20607), ab75+c210 (K20608), ab75+c211 (K20609), ab76+c1 (K20610), ab76+c2 (K20611), ab76+c3 (K20612), ab76+c4 (K20613), ab76+c5 (K20614), ab76+c6 (K20615), ab76+c7 (K20616), ab76+c8 (K20617), ab76+c9 (K20618), ab76+c10 (K20619), ab76+c11 (K20620), ab76+c12 (K20621), ab76+c13 (K20622), ab76+c14 (K20623), ab76+c15 (K20624), ab76+c16 (K20625), ab76+c17 (K20626), ab76+c18 (K20627), ab76+c19 (K20628), ab76+c20 (K20629), ab76+c21 (K20630), ab76+c22 (K20631), ab76+c23 (K20632), ab76+c24 (K20633), ab76+c25 (K20634), ab76+c26 (K20635), ab76+c27 (K20636), ab76+c28 (K20637), ab76+c29 (K20638), ab76+c30 (K20639), ab76+c31 (K20640), ab76+c32 (K20641), ab76+c33 (K20642), ab76+c34 (K20643), ab76+c35 (K20644), ab76+c36 (K20645), ab76+c37 (K20646), ab76+c38 (K20647), ab76+c39 (K20648), ab76+c40 (K20649), ab76+c41 (K20650), ab76+c42 (K20651), ab76+c43 (K20652), ab76+c44 (K20653), ab76+c45 (K20654), ab76+c46 (K20655), ab76+c47 (K20656), ab76+c48 (K20657), ab76+c49 (K20658), ab76+c50 (K20659), ab76+c51 (K20660), ab76+c52 (K20661), ab76+c53 (K20662), ab76+c54 (K20663), ab76+c55 (K20664), ab76+c56 (K20665), ab76+c57 (K20666), ab76+c58 (K20667), ab76+c59 (K20668), ab76+c60 (K20669), ab76+c61 (K20670), ab76+c62 (K20671), ab76+c63 (K20672), ab76+c64 (K20673), ab76+c65 (K20674), ab76+c66 (K20675), ab76+c67 (K20676), ab76+c68 (K20677), ab76+c69 (K20678), ab76+c70 (K20679), ab76+c71 (K20680), ab76+c72 (K20681), ab76+c73 (K20682), ab76+c74 (K20683), ab76+c75 (K20684), ab76+c76 (K20685), ab76+c77 (K20686), ab76+c78 (K20687), ab76+c79 (K20688), ab76+c80 (K20689), ab76+c81 (K20690), ab76+c82 (K20691), ab76+c83 (K20692), ab76+c84 (K20693), ab76+c85 (K20694), ab76+c86 (K20695), ab76+c87 (K20696), ab76+c88 (K20697), ab76+c89 (K20698), ab76+c90 (K20699), ab76+c91 (K20700), ab76+c92 (K20701), ab76+c93 (K20702), ab76+c94 (K20703), ab76+c95 (K20704), ab76+c96 (K20705), ab76+c97 (K20706), ab76+c98 (K20707), ab76+c99 (K20708), ab76+c100 (K20709), ab76+c101 (K20710), ab76+c102 (K20711), ab76+c103 (K20712), ab76+c104 (K20713), ab76+c105 (K20714), ab76+c106 (K20715), ab76+c107 (K20716), ab76+c108 (K20717), ab76+c109 (K20718), ab76+c110 (K20719), ab76+c111 (K20720), ab76+c112 (K20721), ab76+c113 (K20722), ab76+c114 (K20723), ab76+c115 (K20724), ab76+c116 (K20725), ab76+c117 (K20726), ab76+c118 (K20727), ab76+c119 (K20728), ab76+c120 (K20729), ab76+c121 (K20730), ab76+c122 (K20731), ab76+c123 (K20732), ab76+c124 (K20733), ab76+c125 (K20734), ab76+c126 (K20735), ab76+c127 (K20736), ab76+c128 (K20737), ab76+c129 (K20738), ab76+c130 (K20739), ab76+c131 (K20740), ab76+c132 (K20741), ab76+c133 (K20742), ab76+c134 (K20743), ab76+c135 (K20744), ab76+c136 (K20745), ab76+c137 (K20746), ab76+c138 (K20747), ab76+c139 (K20748), ab76+c140 (K20749), ab76+c141 (K20750), ab76+c142 (K20751), ab76+c143 (K20752), ab76+c144 (K20753), ab76+c145 (K20754), ab76+c146 (K20755), ab76+c147 (K20756), ab76+c148 (K20757), ab76+c149 (K20758), ab76+c150 (K20759), ab76+c151 (K20760), ab76+c152 (K20761), ab76+c153 (K20762), ab76+c154 (K20763), ab76+c155 (K20764), ab76+c156 (K20765), ab76+c157 (K20766), ab76+c158 (K20767), ab76+c159 (K20768), ab76+c160 (K20769), ab76+c161 (K20770), ab76+c162 (K20771), ab76+c163 (K20772), ab76+c164 (K20773), ab76+c165 (K20774), ab76+c166 (K20775), ab76+c167 (K20776), ab76+c168 (K20777), ab76+c169 (K20778), ab76+c170 (K20779), ab76+c171 (K20780), ab76+c172 (K20781), ab76+c173 (K20782), ab76+c174 (K20783), ab76+c175 (K20784), ab76+c176 (K20785), ab76+c177 (K20786), ab76+c178 (K20787), ab76+c179 (K20788), ab76+c180 (K20789), ab76+c181 (K20790), ab76+c182 (K20791), ab76+c183 (K20792), ab76+c184 (K20793), ab76+c185 (K20794), ab76+c186 (K20795), ab76+c187 (K20796), ab76+c188 (K20797), ab76+c189 (K20798), ab76+c190 (K20799), ab76+c191 (K20800), ab76+c192 (K20801), ab76+c193 (K20802), ab76+c194 (K20803), ab76+c195 (K20804), ab76+c196 (K20805), ab76+c197 (K20806), ab76+c198 (K20807), ab76+c199 (K20808), ab76+c200 (K20809), ab76+c201 (K20810), ab76+c202 (K20811), ab76+c203 (K20812), ab76+c204 (K20813), ab76+c205 (K20814), ab76+c206 (K20815), ab76+c207 (K20816), ab76+c208 (K20817), ab76+c209 (K20818), ab76+c210 (K20819), and ab76+c211 (K20820), and wherein the [module (a)+module (b)] protein or peptide, the at least one module (c), and the at least one compound (d) are linked to each other in any arrangement. The conjugates of the present invention optionally comprise a nuclear localization signal.

Preferably, the conjugate comprises, essentially consists of, consists of or contains:

(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
wherein the at least one module (a) and the at least one module (b) are comprised or contained within a [module (a)+module (b)] protein or peptide, and wherein the [module (a)+module (b)] protein or peptide and the at least one module (c) are combined in a combination as indicated by a numerical from K1751 to K4358, K5769 to K18141, or K19766 to K20820, and wherein the combination of the [module (a)+module (b)] protein or peptide and the at least one module (c) are combined with the at least one compound (d) according to the following scheme: KX, in each case combined with at least one compound d1; KX, in each case combined with at least one compound d2; KX, in each case combined with at least one compound d3; KX, in each case combined with at least one compound d4; KX, in each case combined with at least one compound d5; KX, in each case combined with at least one compound d6; KX, in each case combined with at least one compound d7; KX, in each case combined with at least one compound d8; KX, in each case combined with at least one compound d9; KX, in each case combined with at least one compound d10; KX, in each case combined with at least one compound d11; KX, in each case combined with at least one compound d12; KX, in each case combined with at least one compound d13; KX, in each case combined with at least one compound d14; KX, in each case combined with at least one compound d15; KX, in each case combined with at least one compound d16; KX, in each case combined with at least one compound d17; KX, in each case combined with at least one compound d18; KX, in each case combined with at least one compound d19; KX, in each case combined with at least one compound d20; KX, in each case combined with at least one compound d21; KX, in each case combined with at least one compound d22; KX, in each case combined with at least one compound d23; KX, in each case combined with at least one compound d24; KX, in each case combined with at least one compound d25; KX, in each case combined with at least one compound d26; KX, in each case combined with at least one compound d27; KX, in each case combined with at least one compound d28; KX, in each case combined with at least one compound d29; KX, in each case combined with at least one compound d30; KX, in each case combined with at least one compound d31; KX, in each case combined with at least one compound d32; KX, in each case combined with at least one compound d33; KX, in each case combined with at least one compound d34; KX, in each case combined with at least one compound d35; KX, in each case combined with at least one compound d36; KX, in each case combined with at least one compound d37; KX, in each case combined with at least one compound d38; KX, in each case combined with at least one compound d39; KX, in each case combined with at least one compound d40; KX, in each case combined with at least one compound d41; KX, in each case combined with at least one compound d42; KX, in each case combined with at least one compound d43; KX, in each case combined with at least one compound d44; KX, in each case combined with at least one compound d45; KX, in each case combined with at least one compound d46; KX, in each case combined with at least one compound d47; KX, in each case combined with at least one compound d48; KX, in each case combined with at least one compound d49; KX, in each case combined with at least one compound d50; KX, in each case combined with at least one compound d51; KX, in each case combined with at least one compound d52; KX, in each case combined with at least one compound d53; KX, in each case combined with at least one compound d54; KX, in each case combined with at least one compound d55; KX, in each case combined with at least one compound d56; KX, in each case combined with at least one compound d57; KX, in each case combined with at least one compound d58; KX, in each case combined with at least one compound d59; KX, in each case combined with at least one compound d60; KX, in each case combined with at least one compound d61; KX, in each case combined with at least one compound d62; KX, in each case combined with at least one compound d63; KX, in each case combined with at least one compound d64; KX, in each case combined with at least one compound d65; KX, in each case combined with at least one compound d66; KX, in each case combined with at least one compound d67; KX, in each case combined with at least one compound d68; KX, in each case combined with at least one compound d69; KX, in each case combined with at least one compound d70; KX, in each case combined with at least one compound d71; KX, in each case combined with at least one compound d72; KX, in each case combined with at least one compound d73; KX, in each case combined with at least one compound d74; KX, in each case combined with at least one compound d75; KX, in each case combined with at least one compound d76; KX, in each case combined with at least one compound d77; KX, in each case combined with at least one compound d78; KX, in each case combined with at least one compound d79; KX, in each case combined with at least one compound d80; KX, in each case combined with at least one compound d81; KX, in each case combined with at least one compound d82; KX, in each case combined with at least one compound d83; KX, in each case combined with at least one compound d84; KX, in each case combined with at least one compound d85; KX, in each case combined with at least one module d86; KX, in each case combined with at least one compound d87; KX, in each case combined with at least one compound d88; KX, in each case combined with at least one compound d89; KX, in each case combined with at least one compound d90; KX, in each case combined with at least one compound d91; KX, in each case combined with at least one compound d92; KX, in each case combined with at least one compound d93; KX, in each case combined with at least one compound d94; KX, in each case combined with at least one compound d95; KX, in each case combined with at least one module d96; KX, in each case combined with at least one compound d97; KX, in each case combined with at least one compound d98; KX, in each case combined with at least one compound d99; KX, in each case combined with at least one compound d100; KX, in each case combined with at least one compound d101; KX, in each case combined with at least one compound d102; KX, in each case combined with at least one compound d103; KX, in each case combined with at least one compound d104; KX, in each case combined with at least one compound d105; KX, in each case combined with at least one compound d106; KX, in each case combined with at least one compound d107; KX, in each case combined with at least one compound d108; KX, in each case combined with at least one compound d109; KX, in each case combined with at least one compound d110; KX, in each case combined with at least one compound d111; KX, in each case combined with at least one compound d112; KX, in each case combined with at least one compound d113; KX, in each case combined with at least one compound d114; KX, in each case combined with at least one compound d115; KX, in each case combined with at least one compound d116; KX, in each case combined with at least one compound d117; KX, in each case combined with at least one compound d118; KX, in each case combined with at least one compound d119; KX, in each case combined with at least one compound d120; KX, in each case combined with at least one compound d121; KX, in each case combined with at least one compound d122; KX, in each case combined with at least one compound d123; KX, in each case combined with at least one compound d124; KX, in each case combined with at least one compound d125; KX, in each case combined with at least one compound d126; KX, in each case combined with at least one compound d127; KX, in each case combined with at least one compound d128; KX, in each case combined with at least one compound d129; KX, in each case combined with at least one compound d130; KX, in each case combined with at least one compound d131; KX, in each case combined with at least one compound d132; KX, in each case combined with at least one compound d133; KX, in each case combined with at least one compound d134; KX, in each case combined with at least one compound d135; KX, in each case combined with at least one compound d136; KX, in each case combined with at least one compound d137; KX, in each case combined with at least one compound d138; KX, in each case combined with at least one compound d139; KX, in each case combined with at least one compound d140; KX, in each case combined with at least one compound d141; KX, in each case combined with at least one compound d142; KX, in each case combined with at least one compound d143; KX, in each case combined with at least one compound d144; KX, in each case combined with at least one compound d145; KX, in each case combined with at least one compound d146; KX, in each case combined with at least one compound d147; KX, in each case combined with at least one compound d148; KX, in each case combined with at least one compound d149; KX, in each case combined with at least one compound d150; KX, in each case combined with at least one compound d151; KX, in each case combined with at least one compound d152; KX, in each case combined with at least one compound d153; KX, in each case combined with at least one compound d154; KX, in each case combined with at least one compound d155; KX, in each case combined with at least one compound d156; KX, in each case combined with at least one compound d157; KX, in each case combined with at least one compound d158; KX, in each case combined with at least one compound d159; KX, in each case combined with at least one compound d160; KX, in each case combined with at least one compound d161; KX, in each case combined with at least one compound d162; KX, in each case combined with at least one compound d163; KX, in each case combined with at least one compound d164; KX, in each case combined with at least one compound d165; KX, in each case combined with at least one compound d166; KX, in each case combined with at least one compound d167; KX, in each case combined with at least one compound d168; KX, in each case combined with at least one compound d169; KX, in each case combined with at least one compound d170;
wherein X is the combination of the [module (a)+module (b)] protein or peptide and at least one module (c) and has the following meaning: 1751, 1752, 1753, 1754, 1755, 1756, 1757, 1758, 1759, 1760, 1761, 1762, 1763, 1764, 1765, 1766, 1767, 1768, 1769, 1770, 1771, 1772, 1773, 1774, 1775, 1776, 1777, 1778, 1779, 1780, 1781, 1782, 1783, 1784, 1785, 1786, 1787, 1788, 1789, 1790, 1791, 1792, 1793, 1794, 1795, 1796, 1797, 1798, 1799, 1800, 1801, 1802, 1803, 1804, 1805, 1806, 1807, 1808, 1809, 1810, 1811, 1812, 1813, 1814, 1815, 1816, 1817, 1818, 1819, 1820, 1821, 1822, 1823, 1824, 1825, 1826, 1827, 1828, 1829, 1830, 1831, 1832, 1833, 1834, 1835, 1836, 1837, 1838, 1839, 1840, 1841, 1842, 1843, 1844, 1845, 1846, 1847, 1848, 1849, 1850, 1851, 1852, 1853, 1854, 1855, 1856, 1857, 1858, 1859, 1860, 1861, 1862, 1863, 1864, 1865, 1866, 1867, 1868, 1869, 1870, 1871, 1872, 1873, 1874, 1875, 1876, 1877, 1878, 1879, 1880, 1881, 1882, 1883, 1884, 1885, 1886, 1887, 1888, 1889, 1890, 1891, 1892, 1893, 1894, 1895, 1896, 1897, 1898, 1899, 1900, 1901, 1902, 1903, 1904, 1905, 1906, 1907, 1908, 1909, 1910, 1911, 1912, 1913, 1914, 1915, 1916, 1917, 1918, 1919, 1920, 1921, 1922, 1923, 1924, 1925, 1926, 1927, 1928, 1929, 1930, 1931, 1932, 1933, 1934, 1935, 1936, 1937, 1938, 1939, 1940, 1941, 1942, 1943, 1944, 1945, 1946, 1947, 1948, 1949, 1950, 1951, 1952, 1953, 1954, 1955, 1956, 1957, 1958, 1959, 1960, 1961, 1962, 1963, 1964, 1965, 1966, 1967, 1968, 1969, 1970, 1971, 1972, 1973, 1974, 1975, 1976, 1977, 1978, 1979, 1980, 1981, 1982, 1983, 1984, 1985, 1986, 1987, 1988, 1989, 1990, 1991, 1992, 1993, 1994, 1995, 1996, 1997, 1998, 1999, 2000, 2001, 2002, 2003, 2004, 2005, 2006, 2007, 2008, 2009, 2010, 2011, 2012, 2013, 2014, 2015, 2016, 2017, 2018, 2019, 2020, 2021, 2022, 2023, 2024, 2025, 2026, 2027, 2028, 2029, 2030, 2031, 2032, 2033, 2034, 2035, 2036, 2037, 2038, 2039, 2040, 2041, 2042, 2043, 2044, 2045, 2046, 2047, 2048, 2049, 2050, 2051, 2052, 2053, 2054, 2055, 2056, 2057, 2058, 2059, 2060, 2061, 2062, 2063, 2064, 2065, 2066, 2067, 2068, 2069, 2070, 2071, 2072, 2073, 2074, 2075, 2076, 2077, 2078, 2079, 2080, 2081, 2082, 2083, 2084, 2085, 2086, 2087, 2088, 2089, 2090, 2091, 2092, 2093, 2094, 2095, 2096, 2097, 2098, 2099, 2100, 2101, 2102, 2103, 2104, 2105, 2106, 2107, 2108, 2109, 2110, 2111, 2112, 2113, 2114, 2115, 2116, 2117, 2118, 2119, 2120, 2121, 2122, 2123, 2124, 2125, 2126, 2127, 2128, 2129, 2130, 2131, 2132, 2133, 2134, 2135, 2136, 2137, 2138, 2139, 2140, 2141, 2142, 2143, 2144, 2145, 2146, 2147, 2148, 2149, 2150, 2151, 2152, 2153, 2154, 2155, 2156, 2157, 2158, 2159, 2160, 2161, 2162, 2163, 2164, 2165, 2166, 2167, 2168, 2169, 2170, 2171, 2172, 2173, 2174, 2175, 2176, 2177, 2178, 2179, 2180, 2181, 2182, 2183, 2184, 2185, 2186, 2187, 2188, 2189, 2190, 2191, 2192, 2193, 2194, 2195, 2196, 2197, 2198, 2199, 2200, 2201, 2202, 2203, 2204, 2205, 2206, 2207, 2208, 2209, 2210, 2211, 2212, 2213, 2214, 2215, 2216, 2217, 2218, 2219, 2220, 2221, 2222, 2223, 2224, 2225, 2226, 2227, 2228, 2229, 2230, 2231, 2232, 2233, 2234, 2235, 2236, 2237, 2238, 2239, 2240, 2241, 2242, 2243, 2244, 2245, 2246, 2247, 2248, 2249, 2250, 2251, 2252, 2253, 2254, 2255, 2256, 2257, 2258, 2259, 2260, 2261, 2262, 2263, 2264, 2265, 2266, 2267, 2268, 2269, 2270, 2271, 2272, 2273, 2274, 2275, 2276, 2277, 2278, 2279, 2280, 2281, 2282, 2283, 2284, 2285, 2286, 2287, 2288, 2289, 2290, 2291, 2292, 2293, 2294, 2295, 2296, 2297, 2298, 2299, 2300, 2301, 2302, 2303, 2304, 2305, 2306, 2307, 2308, 2309, 2310, 2311, 2312, 2313, 2314, 2315, 2316, 2317, 2318, 2319, 2320, 2321, 2322, 2323, 2324, 2325, 2326, 2327, 2328, 2329, 2330, 2331, 2332, 2333, 2334, 2335, 2336, 2337, 2338, 2339, 2340, 2341, 2342, 2343, 2344, 2345, 2346, 2347, 2348, 2349, 2350, 2351, 2352, 2353, 2354, 2355, 2356, 2357, 2358, 2359, 2360, 2361, 2362, 2363, 2364, 2365, 2366, 2367, 2368, 2369, 2370, 2371, 2372, 2373, 2374, 2375, 2376, 2377, 2378, 2379, 2380, 2381, 2382, 2383, 2384, 2385, 2386, 2387, 2388, 2389, 2390, 2391, 2392, 2393, 2394, 2395, 2396, 2397, 2398, 2399, 2400, 2401, 2402, 2403, 2404, 2405, 2406, 2407, 2408, 2409, 2410, 2411, 2412, 2413, 2414, 2415, 2416, 2417, 2418, 2419, 2420, 2421, 2422, 2423, 2424, 2425, 2426, 2427, 2428, 2429, 2430, 2431, 2432, 2433, 2434, 2435, 2436, 2437, 2438, 2439, 2440, 2441, 2442, 2443, 2444, 2445, 2446, 2447, 2448, 2449, 2450, 2451, 2452, 2453, 2454, 2455, 2456, 2457, 2458, 2459, 2460, 2461, 2462, 2463, 2464, 2465, 2466, 2467, 2468, 2469, 2470, 2471, 2472, 2473, 2474, 2475, 2476, 2477, 2478, 2479, 2480, 2481, 2482, 2483, 2484, 2485, 2486, 2487, 2488, 2489, 2490, 2491, 2492, 2493, 2494, 2495, 2496, 2497, 2498, 2499, 2500, 2501, 2502, 2503, 2504, 2505, 2506, 2507, 2508, 2509, 2510, 2511, 2512, 2513, 2514, 2515, 2516, 2517, 2518, 2519, 2520, 2521, 2522, 2523, 2524, 2525, 2526, 2527, 2528, 2529, 2530, 2531, 2532, 2533, 2534, 2535, 2536, 2537, 2538, 2539, 2540, 2541, 2542, 2543, 2544, 2545, 2546, 2547, 2548, 2549, 2550, 2551, 2552, 2553, 2554, 2555, 2556, 2557, 2558, 2559, 2560, 2561, 2562, 2563, 2564, 2565, 2566, 2567, 2568, 2569, 2570, 2571, 2572, 2573, 2574, 2575, 2576, 2577, 2578, 2579, 2580, 2581, 2582, 2583, 2584, 2585, 2586, 2587, 2588, 2589, 2590, 2591, 2592, 2593, 2594, 2595, 2596, 2597, 2598, 2599, 2600, 2601, 2602, 2603, 2604, 2605, 2606, 2607, 2608, 2609, 2610, 2611, 2612, 2613, 2614, 2615, 2616, 2617, 2618, 2619, 2620, 2621, 2622, 2623, 2624, 2625, 2626, 2627, 2628, 2629, 2630, 2631, 2632, 2633, 2634, 2635, 2636, 2637, 2638, 2639, 2640, 2641, 2642, 2643, 2644, 2645, 2646, 2647, 2648, 2649, 2650, 2651, 2652, 2653, 2654, 2655, 2656, 2657, 2658, 2659, 2660, 2661, 2662, 2663, 2664, 2665, 2666, 2667, 2668, 2669, 2670, 2671, 2672, 2673, 2674, 2675, 2676, 2677, 2678, 2679, 2680, 2681, 2682, 2683, 2684, 2685, 2686, 2687, 2688, 2689, 2690, 2691, 2692, 2693, 2694, 2695, 2696, 2697, 2698, 2699, 2700, 2701, 2702, 2703, 2704, 2705, 2706, 2707, 2708, 2709, 2710, 2711, 2712, 2713, 2714, 2715, 2716, 2717, 2718, 2719, 2720, 2721, 2722, 2723, 2724, 2725, 2726, 2727, 2728, 2729, 2730, 2731, 2732, 2733, 2734, 2735, 2736, 2737, 2738, 2739, 2740, 2741, 2742, 2743, 2744, 2745, 2746, 2747, 2748, 2749, 2750, 2751, 2752, 2753, 2754, 2755, 2756, 2757, 2758, 2759, 2760, 2761, 2762, 2763, 2764, 2765, 2766, 2767, 2768, 2769, 2770, 2771, 2772, 2773, 2774, 2775, 2776, 2777, 2778, 2779, 2780, 2781, 2782, 2783, 2784, 2785, 2786, 2787, 2788, 2789, 2790, 2791, 2792, 2793, 2794, 2795, 2796, 2797, 2798, 2799, 2800, 2801, 2802, 2803, 2804, 2805, 2806, 2807, 2808, 2809, 2810, 2811, 2812, 2813, 2814, 2815, 2816, 2817, 2818, 2819, 2820, 2821, 2822, 2823, 2824, 2825, 2826, 2827, 2828, 2829, 2830, 2831, 2832, 2833, 2834, 2835, 2836, 2837, 2838, 2839, 2840, 2841, 2842, 2843, 2844, 2845, 2846, 2847, 2848, 2849, 2850, 2851, 2852, 2853, 2854, 2855, 2856, 2857, 2858, 2859, 2860, 2861, 2862, 2863, 2864, 2865, 2866, 2867, 2868, 2869, 2870, 2871, 2872, 2873, 2874, 2875, 2876, 2877, 2878, 2879, 2880, 2881, 2882, 2883, 2884, 2885, 2886, 2887, 2888, 2889, 2890, 2891, 2892, 2893, 2894, 2895, 2896, 2897, 2898, 2899, 2900, 2901, 2902, 2903, 2904, 2905, 2906, 2907, 2908, 2909, 2910, 2911, 2912, 2913, 2914, 2915, 2916, 2917, 2918, 2919, 2920, 2921, 2922, 2923, 2924, 2925, 2926, 2927, 2928, 2929, 2930, 2931, 2932, 2933, 2934, 2935, 2936, 2937, 2938, 2939, 2940, 2941, 2942, 2943, 2944, 2945, 2946, 2947, 2948, 2949, 2950, 2951, 2952, 2953, 2954, 2955, 2956, 2957, 2958, 2959, 2960, 2961, 2962, 2963, 2964, 2965, 2966, 2967, 2968, 2969, 2970, 2971, 2972, 2973, 2974, 2975, 2976, 2977, 2978, 2979, 2980, 2981, 2982, 2983, 2984, 2985, 2986, 2987, 2988, 2989, 2990, 2991, 2992, 2993, 2994, 2995, 2996, 2997, 2998, 2999, 3000, 3001, 3002, 3003, 3004, 3005, 3006, 3007, 3008, 3009, 3010, 3011, 3012, 3013, 3014, 3015, 3016, 3017, 3018, 3019, 3020, 3021, 3022, 3023, 3024, 3025, 3026, 3027, 3028, 3029, 3030, 3031, 3032, 3033, 3034, 3035, 3036, 3037, 3038, 3039, 3040, 3041, 3042, 3043, 3044, 3045, 3046, 3047, 3048, 3049, 3050, 3051, 3052, 3053, 3054, 3055, 3056, 3057, 3058, 3059, 3060, 3061, 3062, 3063, 3064, 3065, 3066, 3067, 3068, 3069, 3070, 3071, 3072, 3073, 3074, 3075, 3076, 3077, 3078, 3079, 3080, 3081, 3082, 3083, 3084, 3085, 3086, 3087, 3088, 3089, 3090, 3091, 3092, 3093, 3094, 3095, 3096, 3097, 3098, 3099, 3100, 3101, 3102, 3103, 3104, 3105, 3106, 3107, 3108, 3109, 3110, 3111, 3112, 3113, 3114, 3115, 3116, 3117, 3118, 3119, 3120, 3121, 3122, 3123, 3124, 3125, 3126, 3127, 3128, 3129, 3130, 3131, 3132, 3133, 3134, 3135, 3136, 3137, 3138, 3139, 3140, 3141, 3142, 3143, 3144, 3145, 3146, 3147, 3148, 3149, 3150, 3151, 3152, 3153, 3154, 3155, 3156, 3157, 3158, 3159, 3160, 3161, 3162, 3163, 3164, 3165, 3166, 3167, 3168, 3169, 3170, 3171, 3172, 3173, 3174, 3175, 3176, 3177, 3178, 3179, 3180, 3181, 3182, 3183, 3184, 3185, 3186, 3187, 3188, 3189, 3190, 3191, 3192, 3193, 3194, 3195, 3196, 3197, 3198, 3199, 3200, 3201, 3202, 3203, 3204, 3205, 3206, 3207, 3208, 3209, 3210, 3211, 3212, 3213, 3214, 3215, 3216, 3217, 3218, 3219, 3220, 3221, 3222, 3223, 3224, 3225, 3226, 3227, 3228, 3229, 3230, 3231, 3232, 3233, 3234, 3235, 3236, 3237, 3238, 3239, 3240, 3241, 3242, 3243, 3244, 3245, 3246, 3247, 3248, 3249, 3250, 3251, 3252, 3253, 3254, 3255, 3256, 3257, 3258, 3259, 3260, 3261, 3262, 3263, 3264, 3265, 3266, 3267, 3268, 3269, 3270, 3271, 3272, 3273, 3274, 3275, 3276, 3277, 3278, 3279, 3280, 3281, 3282, 3283, 3284, 3285, 3286, 3287, 3288, 3289, 3290, 3291, 3292, 3293, 3294, 3295, 3296, 3297, 3298, 3299, 3300, 3301, 3302, 3303, 3304, 3305, 3306, 3307, 3308, 3309, 3310, 3311, 3312, 3313, 3314, 3315, 3316, 3317, 3318, 3319, 3320, 3321, 3322, 3323, 3324, 3325, 3326, 3327, 3328, 3329, 3330, 3331, 3332, 3333, 3334, 3335, 3336, 3337, 3338, 3339, 3340, 3341, 3342, 3343, 3344, 3345, 3346, 3347, 3348, 3349, 3350, 3351, 3352, 3353, 3354, 3355, 3356, 3357, 3358, 3359, 3360, 3361, 3362, 3363, 3364, 3365, 3366, 3367, 3368, 3369, 3370, 3371, 3372, 3373, 3374, 3375, 3376, 3377, 3378, 3379, 3380, 3381, 3382, 3383, 3384, 3385, 3386, 3387, 3388, 3389, 3390, 3391, 3392, 3393, 3394, 3395, 3396, 3397, 3398, 3399, 3400, 3401, 3402, 3403, 3404, 3405, 3406, 3407, 3408, 3409, 3410, 3411, 3412, 3413, 3414, 3415, 3416, 3417, 3418, 3419, 3420, 3421, 3422, 3423, 3424, 3425, 3426, 3427, 3428, 3429, 3430, 3431, 3432, 3433, 3434, 3435, 3436, 3437, 3438, 3439, 3440, 3441, 3442, 3443, 3444, 3445, 3446, 3447, 3448, 3449, 3450, 3451, 3452, 3453, 3454, 3455, 3456, 3457, 3458, 3459, 3460, 3461, 3462, 3463, 3464, 3465, 3466, 3467, 3468, 3469, 3470, 3471, 3472, 3473, 3474, 3475, 3476, 3477, 3478, 3479, 3480, 3481, 3482, 3483, 3484, 3485, 3486, 3487, 3488, 3489, 3490, 3491, 3492, 3493, 3494, 3495, 3496, 3497, 3498, 3499, 3500, 3501, 3502, 3503, 3504, 3505, 3506, 3507, 3508, 3509, 3510, 3511, 3512, 3513, 3514, 3515, 3516, 3517, 3518, 3519, 3520, 3521, 3522, 3523, 3524, 3525, 3526, 3527, 3528, 3529, 3530, 3531, 3532, 3533, 3534, 3535, 3536, 3537, 3538, 3539, 3540, 3541, 3542, 3543, 3544, 3545, 3546, 3547, 3548, 3549, 3550, 3551, 3552, 3553, 3554, 3555, 3556, 3557, 3558, 3559, 3560, 3561, 3562, 3563, 3564, 3565, 3566, 3567, 3568, 3569, 3570, 3571, 3572, 3573, 3574, 3575, 3576, 3577, 3578, 3579, 3580, 3581, 3582, 3583, 3584, 3585, 3586, 3587, 3588, 3589, 3590, 3591, 3592, 3593, 3594, 3595, 3596, 3597, 3598, 3599, 3600, 3601, 3602, 3603, 3604, 3605, 3606, 3607, 3608, 3609, 3610, 3611, 3612, 3613, 3614, 3615, 3616, 3617, 3618, 3619, 3620, 3621, 3622, 3623, 3624, 3625, 3626, 3627, 3628, 3629, 3630, 3631, 3632, 3633, 3634, 3635, 3636, 3637, 3638, 3639, 3640, 3641, 3642, 3643, 3644, 3645, 3646, 3647, 3648, 3649, 3650, 3651, 3652, 3653, 3654, 3655, 3656, 3657, 3658, 3659, 3660, 3661, 3662, 3663, 3664, 3665, 3666, 3667, 3668, 3669, 3670, 3671, 3672, 3673, 3674, 3675, 3676, 3677, 3678, 3679, 3680, 3681, 3682, 3683, 3684, 3685, 3686, 3687, 3688, 3689, 3690, 3691, 3692, 3693, 3694, 3695, 3696, 3697, 3698, 3699, 3700, 3701, 3702, 3703, 3704, 3705, 3706, 3707, 3708, 3709, 3710, 3711, 3712, 3713, 3714, 3715, 3716, 3717, 3718, 3719, 3720, 3721, 3722, 3723, 3724, 3725, 3726, 3727, 3728, 3729, 3730, 3731, 3732, 3733, 3734, 3735, 3736, 3737, 3738, 3739, 3740, 3741, 3742, 3743, 3744, 3745, 3746, 3747, 3748, 3749, 3750, 3751, 3752, 3753, 3754, 3755, 3756, 3757, 3758, 3759, 3760, 3761, 3762, 3763, 3764, 3765, 3766, 3767, 3768, 3769, 3770, 3771, 3772, 3773, 3774, 3775, 3776, 3777, 3778, 3779, 3780, 3781, 3782, 3783, 3784, 3785, 3786, 3787, 3788, 3789, 3790, 3791, 3792, 3793, 3794, 3795, 3796, 3797, 3798, 3799, 3800, 3801, 3802, 3803, 3804, 3805, 3806, 3807, 3808, 3809, 3810, 3811, 3812, 3813, 3814, 3815, 3816, 3817, 3818, 3819, 3820, 3821, 3822, 3823, 3824, 3825, 3826, 3827, 3828, 3829, 3830, 3831, 3832, 3833, 3834, 3835, 3836, 3837, 3838, 3839, 3840, 3841, 3842, 3843, 3844, 3845, 3846, 3847, 3848, 3849, 3850, 3851, 3852, 3853, 3854, 3855, 3856, 3857, 3858, 3859, 3860, 3861, 3862, 3863, 3864, 3865, 3866, 3867, 3868, 3869, 3870, 3871, 3872, 3873, 3874, 3875, 3876, 3877, 3878, 3879, 3880, 3881, 3882, 3883, 3884, 3885, 3886, 3887, 3888, 3889, 3890, 3891, 3892, 3893, 3894, 3895, 3896, 3897, 3898, 3899, 3900, 3901, 3902, 3903, 3904, 3905, 3906, 3907, 3908, 3909, 3910, 3911, 3912, 3913, 3914, 3915, 3916, 3917, 3918, 3919, 3920, 3921, 3922, 3923, 3924, 3925, 3926, 3927, 3928, 3929, 3930, 3931, 3932, 3933, 3934, 3935, 3936, 3937, 3938, 3939, 3940, 3941, 3942, 3943, 3944, 3945, 3946, 3947, 3948, 3949, 3950, 3951, 3952, 3953, 3954, 3955, 3956, 3957, 3958, 3959, 3960, 3961, 3962, 3963, 3964, 3965, 3966, 3967, 3968, 3969, 3970, 3971, 3972, 3973, 3974, 3975, 3976, 3977, 3978, 3979, 3980, 3981, 3982, 3983, 3984, 3985, 3986, 3987, 3988, 3989, 3990, 3991, 3992, 3993, 3994, 3995, 3996, 3997, 3998, 3999, 4000, 4001, 4002, 4003, 4004, 4005, 4006, 4007, 4008, 4009, 4010, 4011, 4012, 4013, 4014, 4015, 4016, 4017, 4018, 4019, 4020, 4021, 4022, 4023, 4024, 4025, 4026, 4027, 4028, 4029, 4030, 4031, 4032, 4033, 4034, 4035, 4036, 4037, 4038, 4039, 4040, 4041, 4042, 4043, 4044, 4045, 4046, 4047, 4048, 4049, 4050, 4051, 4052, 4053, 4054, 4055, 4056, 4057, 4058, 4059, 4060, 4061, 4062, 4063, 4064, 4065, 4066, 4067, 4068, 4069, 4070, 4071, 4072, 4073, 4074, 4075, 4076, 4077, 4078, 4079, 4080, 4081, 4082, 4083, 4084, 4085, 4086, 4087, 4088, 4089, 4090, 4091, 4092, 4093, 4094, 4095, 4096, 4097, 4098, 4099, 4100, 4101, 4102, 4103, 4104, 4105, 4106, 4107, 4108, 4109, 4110, 4111, 4112, 4113, 4114, 4115, 4116, 4117, 4118, 4119, 4120, 4121, 4122, 4123, 4124, 4125, 4126, 4127, 4128, 4129, 4130, 4131, 4132, 4133, 4134, 4135, 4136, 4137, 4138, 4139, 4140, 4141, 4142, 4143, 4144, 4145, 4146, 4147, 4148, 4149, 4150, 4151, 4152, 4153, 4154, 4155, 4156, 4157, 4158, 4159, 4160, 4161, 4162, 4163, 4164, 4165, 4166, 4167, 4168, 4169, 4170, 4171, 4172, 4173, 4174, 4175, 4176, 4177, 4178, 4179, 4180, 4181, 4182, 4183, 4184, 4185, 4186, 4187, 4188, 4189, 4190, 4191, 4192, 4193, 4194, 4195, 4196, 4197, 4198, 4199, 4200, 4201, 4202, 4203, 4204, 4205, 4206, 4207, 4208, 4209, 4210, 4211, 4212, 4213, 4214, 4215, 4216, 4217, 4218, 4219, 4220, 4221, 4222, 4223, 4224, 4225, 4226, 4227, 4228, 4229, 4230, 4231, 4232, 4233, 4234, 4235, 4236, 4237, 4238, 4239, 4240, 4241, 4242, 4243, 4244, 4245, 4246, 4247, 4248, 4249, 4250, 4251, 4252, 4253, 4254, 4255, 4256, 4257, 4258, 4259, 4260, 4261, 4262, 4263, 4264, 4265, 4266, 4267, 4268, 4269, 4270, 4271, 4272, 4273, 4274, 4275, 4276, 4277, 4278, 4279, 4280, 4281, 4282, 4283, 4284, 4285, 4286, 4287, 4288, 4289, 4290, 4291, 4292, 4293, 4294, 4295, 4296, 4297, 4298, 4299, 4300, 4301, 4302, 4303, 4304, 4305, 4306, 4307, 4308, 4309, 4310, 4311, 4312, 4313, 4314, 4315, 4316, 4317, 4318, 4319, 4320, 4321, 4322, 4323, 4324, 4325, 4326, 4327, 4328, 4329, 4330, 4331, 4332, 4333, 4334, 4335, 4336, 4337, 4338, 4339, 4340, 4341, 4342, 4343, 4344, 4345, 4346, 4347, 4348, 4349, 4350, 4351, 4352, 4353, 4354, 4355, 4356, 4357, 4358, 5769, 5770, 5771, 5772, 5773, 5774, 5775, 5776, 5777, 5778, 5779, 5780, 5781, 5782, 5783, 5784, 5785, 5786, 5787, 5788, 5789, 5790, 5791, 5792, 5793, 5794, 5795, 5796, 5797, 5798, 5799, 5800, 5801, 5802, 5803, 5804, 5805, 5806, 5807, 5808, 5809, 5810, 5811, 5812, 5813, 5814, 5815, 5816, 5817, 5818, 5819, 5820, 5821, 5822, 5823, 5824, 5825, 5826, 5827, 5828, 5829, 5830, 5831, 5832, 5833, 5834, 5835, 5836, 5837, 5838, 5839, 5840, 5841, 5842, 5843, 5844, 5845, 5846, 5847, 5848, 5849, 5850, 5851, 5852, 5853, 5854, 5855, 5856, 5857, 5858, 5859, 5860, 5861, 5862, 5863, 5864, 5865, 5866, 5867, 5868, 5869, 5870, 5871, 5872, 5873, 5874, 5875, 5876, 5877, 5878, 5879, 5880, 5881, 5882, 5883, 5884, 5885, 5886, 5887, 5888, 5889, 5890, 5891, 5892, 5893, 5894, 5895, 5896, 5897, 5898, 5899, 5900, 5901, 5902, 5903, 5904, 5905, 5906, 5907, 5908, 5909, 5910, 5911, 5912, 5913, 5914, 5915, 5916, 5917, 5918, 5919, 5920, 5921, 5922, 5923, 5924, 5925, 5926, 5927, 5928, 5929, 5930, 5931, 5932, 5933, 5934, 5935, 5936, 5937, 5938, 5939, 5940, 5941, 5942, 5943, 5944, 5945, 5946, 5947, 5948, 5949, 5950, 5951, 5952, 5953, 5954, 5955, 5956, 5957, 5958, 5959, 5960, 5961, 5962, 5963, 5964, 5965, 5966, 5967, 5968, 5969, 5970, 5971, 5972, 5973, 5974, 5975, 5976, 5977, 5978, 5979, 5980, 5981, 5982, 5983, 5984, 5985, 5986, 5987, 5988, 5989, 5990, 5991, 5992, 5993, 5994, 5995, 5996, 5997, 5998, 5999, 6000, 6001, 6002, 6003, 6004, 6005, 6006, 6007, 6008, 6009, 6010, 6011, 6012, 6013, 6014, 6015, 6016, 6017, 6018, 6019, 6020, 6021, 6022, 6023, 6024, 6025, 6026, 6027, 6028, 6029, 6030, 6031, 6032, 6033, 6034, 6035, 6036, 6037, 6038, 6039, 6040, 6041, 6042, 6043, 6044, 6045, 6046, 6047, 6048, 6049, 6050, 6051, 6052, 6053, 6054, 6055, 6056, 6057, 6058, 6059, 6060, 6061, 6062, 6063, 6064, 6065, 6066, 6067, 6068, 6069, 6070, 6071, 6072, 6073, 6074, 6075, 6076, 6077, 6078, 6079, 6080, 6081, 6082, 6083, 6084, 6085, 6086, 6087, 6088, 6089, 6090, 6091, 6092, 6093, 6094, 6095, 6096, 6097, 6098, 6099, 6100, 6101, 6102, 6103, 6104, 6105, 6106, 6107, 6108, 6109, 6110, 6111, 6112, 6113, 6114, 6115, 6116, 6117, 6118, 6119, 6120, 6121, 6122, 6123, 6124, 6125, 6126, 6127, 6128, 6129, 6130, 6131, 6132, 6133, 6134, 6135, 6136, 6137, 6138, 6139, 6140, 6141, 6142, 6143, 6144, 6145, 6146, 6147, 6148, 6149, 6150, 6151, 6152, 6153, 6154, 6155, 6156, 6157, 6158, 6159, 6160, 6161, 6162, 6163, 6164, 6165, 6166, 6167, 6168, 6169, 6170, 6171, 6172, 6173, 6174, 6175, 6176, 6177, 6178, 6179, 6180, 6181, 6182, 6183, 6184, 6185, 6186, 6187, 6188, 6189, 6190, 6191, 6192, 6193, 6194, 6195, 6196, 6197, 6198, 6199, 6200, 6201, 6202, 6203, 6204, 6205, 6206, 6207, 6208, 6209, 6210, 6211, 6212, 6213, 6214, 6215, 6216, 6217, 6218, 6219, 6220, 6221, 6222, 6223, 6224, 6225, 6226, 6227, 6228, 6229, 6230, 6231, 6232, 6233, 6234, 6235, 6236, 6237, 6238, 6239, 6240, 6241, 6242, 6243, 6244, 6245, 6246, 6247, 6248, 6249, 6250, 6251, 6252, 6253, 6254, 6255, 6256, 6257, 6258, 6259, 6260, 6261, 6262, 6263, 6264, 6265, 6266, 6267, 6268, 6269, 6270, 6271, 6272, 6273, 6274, 6275, 6276, 6277, 6278, 6279, 6280, 6281, 6282, 6283, 6284, 6285, 6286, 6287, 6288, 6289, 6290, 6291, 6292, 6293, 6294, 6295, 6296, 6297, 6298, 6299, 6300, 6301, 6302, 6303, 6304, 6305, 6306, 6307, 6308, 6309, 6310, 6311, 6312, 6313, 6314, 6315, 6316, 6317, 6318, 6319, 6320, 6321, 6322, 6323, 6324, 6325, 6326, 6327, 6328, 6329, 6330, 6331, 6332, 6333, 6334, 6335, 6336, 6337, 6338, 6339, 6340, 6341, 6342, 6343, 6344, 6345, 6346, 6347, 6348, 6349, 6350, 6351, 6352, 6353, 6354, 6355, 6356, 6357, 6358, 6359, 6360, 6361, 6362, 6363, 6364, 6365, 6366, 6367, 6368, 6369, 6370, 6371, 6372, 6373, 6374, 6375, 6376, 6377, 6378, 6379, 6380, 6381, 6382, 6383, 6384, 6385, 6386, 6387, 6388, 6389, 6390, 6391, 6392, 6393, 6394, 6395, 6396, 6397, 6398, 6399, 6400, 6401, 6402, 6403, 6404, 6405, 6406, 6407, 6408, 6409, 6410, 6411, 6412, 6413, 6414, 6415, 6416, 6417, 6418, 6419, 6420, 6421, 6422, 6423, 6424, 6425, 6426, 6427, 6428, 6429, 6430, 6431, 6432, 6433, 6434, 6435, 6436, 6437, 6438, 6439, 6440, 6441, 6442, 6443, 6444, 6445, 6446, 6447, 6448, 6449, 6450, 6451, 6452, 6453, 6454, 6455, 6456, 6457, 6458, 6459, 6460, 6461, 6462, 6463, 6464, 6465, 6466, 6467, 6468, 6469, 6470, 6471, 6472, 6473, 6474, 6475, 6476, 6477, 6478, 6479, 6480, 6481, 6482, 6483, 6484, 6485, 6486, 6487, 6488, 6489, 6490, 6491, 6492, 6493, 6494, 6495, 6496, 6497, 6498, 6499, 6500, 6501, 6502, 6503, 6504, 6505, 6506, 6507, 6508, 6509, 6510, 6511, 6512, 6513, 6514, 6515, 6516, 6517, 6518, 6519, 6520, 6521, 6522, 6523, 6524, 6525, 6526, 6527, 6528, 6529, 6530, 6531, 6532, 6533, 6534, 6535, 6536, 6537, 6538, 6539, 6540, 6541, 6542, 6543, 6544, 6545, 6546, 6547, 6548, 6549, 6550, 6551, 6552, 6553, 6554, 6555, 6556, 6557, 6558, 6559, 6560, 6561, 6562, 6563, 6564, 6565, 6566, 6567, 6568, 6569, 6570, 6571, 6572, 6573, 6574, 6575, 6576, 6577, 6578, 6579, 6580, 6581, 6582, 6583, 6584, 6585, 6586, 6587, 6588, 6589, 6590, 6591, 6592, 6593, 6594, 6595, 6596, 6597, 6598, 6599, 6600, 6601, 6602, 6603, 6604, 6605, 6606, 6607, 6608, 6609, 6610, 6611, 6612, 6613, 6614, 6615, 6616, 6617, 6618, 6619, 6620, 6621, 6622, 6623, 6624, 6625, 6626, 6627, 6628, 6629, 6630, 6631, 6632, 6633, 6634, 6635, 6636, 6637, 6638, 6639, 6640, 6641, 6642, 6643, 6644, 6645, 6646, 6647, 6648, 6649, 6650, 6651, 6652, 6653, 6654, 6655, 6656, 6657, 6658, 6659, 6660, 6661, 6662, 6663, 6664, 6665, 6666, 6667, 6668, 6669, 6670, 6671, 6672, 6673, 6674, 6675, 6676, 6677, 6678, 6679, 6680, 6681, 6682, 6683, 6684, 6685, 6686, 6687, 6688, 6689, 6690, 6691, 6692, 6693, 6694, 6695, 6696, 6697, 6698, 6699, 6700, 6701, 6702, 6703, 6704, 6705, 6706, 6707, 6708, 6709, 6710, 6711, 6712, 6713, 6714, 6715, 6716, 6717, 6718, 6719, 6720, 6721, 6722, 6723, 6724, 6725, 6726, 6727, 6728, 6729, 6730, 6731, 6732, 6733, 6734, 6735, 6736, 6737, 6738, 6739, 6740, 6741, 6742, 6743, 6744, 6745, 6746, 6747, 6748, 6749, 6750, 6751, 6752, 6753, 6754, 6755, 6756, 6757, 6758, 6759, 6760, 6761, 6762, 6763, 6764, 6765, 6766, 6767, 6768, 6769, 6770, 6771, 6772, 6773, 6774, 6775, 6776, 6777, 6778, 6779, 6780, 6781, 6782, 6783, 6784, 6785, 6786, 6787, 6788, 6789, 6790, 6791, 6792, 6793, 6794, 6795, 6796, 6797, 6798, 6799, 6800, 6801, 6802, 6803, 6804, 6805, 6806, 6807, 6808, 6809, 6810, 6811, 6812, 6813, 6814, 6815, 6816, 6817, 6818, 6819, 6820, 6821, 6822, 6823, 6824, 6825, 6826, 6827, 6828, 6829, 6830, 6831, 6832, 6833, 6834, 6835, 6836, 6837, 6838, 6839, 6840, 6841, 6842, 6843, 6844, 6845, 6846, 6847, 6848, 6849, 6850, 6851, 6852, 6853, 6854, 6855, 6856, 6857, 6858, 6859, 6860, 6861, 6862, 6863, 6864, 6865, 6866, 6867, 6868, 6869, 6870, 6871, 6872, 6873, 6874, 6875, 6876, 6877, 6878, 6879, 6880, 6881, 6882, 6883, 6884, 6885, 6886, 6887, 6888, 6889, 6890, 6891, 6892, 6893, 6894, 6895, 6896, 6897, 6898, 6899, 6900, 6901, 6902, 6903, 6904, 6905, 6906, 6907, 6908, 6909, 6910, 6911, 6912, 6913, 6914, 6915, 6916, 6917, 6918, 6919, 6920, 6921, 6922, 6923, 6924, 6925, 6926, 6927, 6928, 6929, 6930, 6931, 6932, 6933, 6934, 6935, 6936, 6937, 6938, 6939, 6940, 6941, 6942, 6943, 6944, 6945, 6946, 6947, 6948, 6949, 6950, 6951, 6952, 6953, 6954, 6955, 6956, 6957, 6958, 6959, 6960, 6961, 6962, 6963, 6964, 6965, 6966, 6967, 6968, 6969, 6970, 6971, 6972, 6973, 6974, 6975, 6976, 6977, 6978, 6979, 6980, 6981, 6982, 6983, 6984, 6985, 6986, 6987, 6988, 6989, 6990, 6991, 6992, 6993, 6994, 6995, 6996, 6997, 6998, 6999, 7000, 7001, 7002, 7003, 7004, 7005, 7006, 7007, 7008, 7009, 7010, 7011, 7012, 7013, 7014, 7015, 7016, 7017, 7018, 7019, 7020, 7021, 7022, 7023, 7024, 7025, 7026, 7027, 7028, 7029, 7030, 7031, 7032, 7033, 7034, 7035, 7036, 7037, 7038, 7039, 7040, 7041, 7042, 7043, 7044, 7045, 7046, 7047, 7048, 7049, 7050, 7051, 7052, 7053, 7054, 7055, 7056, 7057, 7058, 7059, 7060, 7061, 7062, 7063, 7064, 7065, 7066, 7067, 7068, 7069, 7070, 7071, 7072, 7073, 7074, 7075, 7076, 7077, 7078, 7079, 7080, 7081, 7082, 7083, 7084, 7085, 7086, 7087, 7088, 7089, 7090, 7091, 7092, 7093, 7094, 7095, 7096, 7097, 7098, 7099, 7100, 7101, 7102, 7103, 7104, 7105, 7106, 7107, 7108, 7109, 7110, 7111, 7112, 7113, 7114, 7115, 7116, 7117, 7118, 7119, 7120, 7121, 7122, 7123, 7124, 7125, 7126, 7127, 7128, 7129, 7130, 7131, 7132, 7133, 7134, 7135, 7136, 7137, 7138, 7139, 7140, 7141, 7142, 7143, 7144, 7145, 7146, 7147, 7148, 7149, 7150, 7151, 7152, 7153, 7154, 7155, 7156, 7157, 7158, 7159, 7160, 7161, 7162, 7163, 7164, 7165, 7166, 7167, 7168, 7169, 7170, 7171, 7172, 7173, 7174, 7175, 7176, 7177, 7178, 7179, 7180, 7181, 7182, 7183, 7184, 7185, 7186, 7187, 7188, 7189, 7190, 7191, 7192, 7193, 7194, 7195, 7196, 7197, 7198, 7199, 7200, 7201, 7202, 7203, 7204, 7205, 7206, 7207, 7208, 7209, 7210, 7211, 7212, 7213, 7214, 7215, 7216, 7217, 7218, 7219, 7220, 7221, 7222, 7223, 7224, 7225, 7226, 7227, 7228, 7229, 7230, 7231, 7232, 7233, 7234, 7235, 7236, 7237, 7238, 7239, 7240, 7241, 7242, 7243, 7244, 7245, 7246, 7247, 7248, 7249, 7250, 7251, 7252, 7253, 7254, 7255, 7256, 7257, 7258, 7259, 7260, 7261, 7262, 7263, 7264, 7265, 7266, 7267, 7268, 7269, 7270, 7271, 7272, 7273, 7274, 7275, 7276, 7277, 7278, 7279, 7280, 7281, 7282, 7283, 7284, 7285, 7286, 7287, 7288, 7289, 7290, 7291, 7292, 7293, 7294, 7295, 7296, 7297, 7298, 7299, 7300, 7301, 7302, 7303, 7304, 7305, 7306, 7307, 7308, 7309, 7310, 7311, 7312, 7313, 7314, 7315, 7316, 7317, 7318, 7319, 7320, 7321, 7322, 7323, 7324, 7325, 7326, 7327, 7328, 7329, 7330, 7331, 7332, 7333, 7334, 7335, 7336, 7337, 7338, 7339, 7340, 7341, 7342, 7343, 7344, 7345, 7346, 7347, 7348, 7349, 7350, 7351, 7352, 7353, 7354, 7355, 7356, 7357, 7358, 7359, 7360, 7361, 7362, 7363, 7364, 7365, 7366, 7367, 7368, 7369, 7370, 7371, 7372, 7373, 7374, 7375, 7376, 7377, 7378, 7379, 7380, 7381, 7382, 7383, 7384, 7385, 7386, 7387, 7388, 7389, 7390, 7391, 7392, 7393, 7394, 7395, 7396, 7397, 7398, 7399, 7400, 7401, 7402, 7403, 7404, 7405, 7406, 7407, 7408, 7409, 7410, 7411, 7412, 7413, 7414, 7415, 7416, 7417, 7418, 7419, 7420, 7421, 7422, 7423, 7424, 7425, 7426, 7427, 7428, 7429, 7430, 7431, 7432, 7433, 7434, 7435, 7436, 7437, 7438, 7439, 7440, 7441, 7442, 7443, 7444, 7445, 7446, 7447, 7448, 7449, 7450, 7451, 7452, 7453, 7454, 7455, 7456, 7457, 7458, 7459, 7460, 7461, 7462, 7463, 7464, 7465, 7466, 7467, 7468, 7469, 7470, 7471, 7472, 7473, 7474, 7475, 7476, 7477, 7478, 7479, 7480, 7481, 7482, 7483, 7484, 7485, 7486, 7487, 7488, 7489, 7490, 7491, 7492, 7493, 7494, 7495, 7496, 7497, 7498, 7499, 7500, 7501, 7502, 7503, 7504, 7505, 7506, 7507, 7508, 7509, 7510, 7511, 7512, 7513, 7514, 7515, 7516, 7517, 7518, 7519, 7520, 7521, 7522, 7523, 7524, 7525, 7526, 7527, 7528, 7529, 7530, 7531, 7532, 7533, 7534, 7535, 7536, 7537, 7538, 7539, 7540, 7541, 7542, 7543, 7544, 7545, 7546, 7547, 7548, 7549, 7550, 7551, 7552, 7553, 7554, 7555, 7556, 7557, 7558, 7559, 7560, 7561, 7562, 7563, 7564, 7565, 7566, 7567, 7568, 7569, 7570, 7571, 7572, 7573, 7574, 7575, 7576, 7577, 7578, 7579, 7580, 7581, 7582, 7583, 7584, 7585, 7586, 7587, 7588, 7589, 7590, 7591, 7592, 7593, 7594, 7595, 7596, 7597, 7598, 7599, 7600, 7601, 7602, 7603, 7604, 7605, 7606, 7607, 7608, 7609, 7610, 7611, 7612, 7613, 7614, 7615, 7616, 7617, 7618, 7619, 7620, 7621, 7622, 7623, 7624, 7625, 7626, 7627, 7628, 7629, 7630, 7631, 7632, 7633, 7634, 7635, 7636, 7637, 7638, 7639, 7640, 7641, 7642, 7643, 7644, 7645, 7646, 7647, 7648, 7649, 7650, 7651, 7652, 7653, 7654, 7655, 7656, 7657, 7658, 7659, 7660, 7661, 7662, 7663, 7664, 7665, 7666, 7667, 7668, 7669, 7670, 7671, 7672, 7673, 7674, 7675, 7676, 7677, 7678, 7679, 7680, 7681, 7682, 7683, 7684, 7685, 7686, 7687, 7688, 7689, 7690, 7691, 7692, 7693, 7694, 7695, 7696, 7697, 7698, 7699, 7700, 7701, 7702, 7703, 7704, 7705, 7706, 7707, 7708, 7709, 7710, 7711, 7712, 7713, 7714, 7715, 7716, 7717, 7718, 7719, 7720, 7721, 7722, 7723, 7724, 7725, 7726, 7727, 7728, 7729, 7730, 7731, 7732, 7733, 7734, 7735, 7736, 7737, 7738, 7739, 7740, 7741, 7742, 7743, 7744, 7745, 7746, 7747, 7748, 7749, 7750, 7751, 7752, 7753, 7754, 7755, 7756, 7757, 7758, 7759, 7760, 7761, 7762, 7763, 7764, 7765, 7766, 7767, 7768, 7769, 7770, 7771, 7772, 7773, 7774, 7775, 7776, 7777, 7778, 7779, 7780, 7781, 7782, 7783, 7784, 7785, 7786, 7787, 7788, 7789, 7790, 7791, 7792, 7793, 7794, 7795, 7796, 7797, 7798, 7799, 7800, 7801, 7802, 7803, 7804, 7805, 7806, 7807, 7808, 7809, 7810, 7811, 7812, 7813, 7814, 7815, 7816, 7817, 7818, 7819, 7820, 7821, 7822, 7823, 7824, 7825, 7826, 7827, 7828, 7829, 7830, 7831, 7832, 7833, 7834, 7835, 7836, 7837, 7838, 7839, 7840, 7841, 7842, 7843, 7844, 7845, 7846, 7847, 7848, 7849, 7850, 7851, 7852, 7853, 7854, 7855, 7856, 7857, 7858, 7859, 7860, 7861, 7862, 7863, 7864, 7865, 7866, 7867, 7868, 7869, 7870, 7871, 7872, 7873, 7874, 7875, 7876, 7877, 7878, 7879, 7880, 7881, 7882, 7883, 7884, 7885, 7886, 7887, 7888, 7889, 7890, 7891, 7892, 7893, 7894, 7895, 7896, 7897, 7898, 7899, 7900, 7901, 7902, 7903, 7904, 7905, 7906, 7907, 7908, 7909, 7910, 7911, 7912, 7913, 7914, 7915, 7916, 7917, 7918, 7919, 7920, 7921, 7922, 7923, 7924, 7925, 7926, 7927, 7928, 7929, 7930, 7931, 7932, 7933, 7934, 7935, 7936, 7937, 7938, 7939, 7940, 7941, 7942, 7943, 7944, 7945, 7946, 7947, 7948, 7949, 7950, 7951, 7952, 7953, 7954, 7955, 7956, 7957, 7958, 7959, 7960, 7961, 7962, 7963, 7964, 7965, 7966, 7967, 7968, 7969, 7970, 7971, 7972, 7973, 7974, 7975, 7976, 7977, 7978, 7979, 7980, 7981, 7982, 7983, 7984, 7985, 7986, 7987, 7988, 7989, 7990, 7991, 7992, 7993, 7994, 7995, 7996, 7997, 7998, 7999, 8000, 8001, 8002, 8003, 8004, 8005, 8006, 8007, 8008, 8009, 8010, 8011, 8012, 8013, 8014, 8015, 8016, 8017, 8018, 8019, 8020, 8021, 8022, 8023, 8024, 8025, 8026, 8027, 8028, 8029, 8030, 8031, 8032, 8033, 8034, 8035, 8036, 8037, 8038, 8039, 8040, 8041, 8042, 8043, 8044, 8045, 8046, 8047, 8048, 8049, 8050, 8051, 8052, 8053, 8054, 8055, 8056, 8057, 8058, 8059, 8060, 8061, 8062, 8063, 8064, 8065, 8066, 8067, 8068, 8069, 8070, 8071, 8072, 8073, 8074, 8075, 8076, 8077, 8078, 8079, 8080, 8081, 8082, 8083, 8084, 8085, 8086, 8087, 8088, 8089, 8090, 8091, 8092, 8093, 8094, 8095, 8096, 8097, 8098, 8099, 8100, 8101, 8102, 8103, 8104, 8105, 8106, 8107, 8108, 8109, 8110, 8111, 8112, 8113, 8114, 8115, 8116, 8117, 8118, 8119, 8120, 8121, 8122, 8123, 8124, 8125, 8126, 8127, 8128, 8129, 8130, 8131, 8132, 8133, 8134, 8135, 8136, 8137, 8138, 8139, 8140, 8141, 8142, 8143, 8144, 8145, 8146, 8147, 8148, 8149, 8150, 8151, 8152, 8153, 8154, 8155, 8156, 8157, 8158, 8159, 8160, 8161, 8162, 8163, 8164, 8165, 8166, 8167, 8168, 8169, 8170, 8171, 8172, 8173, 8174, 8175, 8176, 8177, 8178, 8179, 8180, 8181, 8182, 8183, 8184, 8185, 8186, 8187, 8188, 8189, 8190, 8191, 8192, 8193, 8194, 8195, 8196, 8197, 8198, 8199, 8200, 8201, 8202, 8203, 8204, 8205, 8206, 8207, 8208, 8209, 8210, 8211, 8212, 8213, 8214, 8215, 8216, 8217, 8218, 8219, 8220, 8221, 8222, 8223, 8224, 8225, 8226, 8227, 8228, 8229, 8230, 8231, 8232, 8233, 8234, 8235, 8236, 8237, 8238, 8239, 8240, 8241, 8242, 8243, 8244, 8245, 8246, 8247, 8248, 8249, 8250, 8251, 8252, 8253, 8254, 8255, 8256, 8257, 8258, 8259, 8260, 8261, 8262, 8263, 8264, 8265, 8266, 8267, 8268, 8269, 8270, 8271, 8272, 8273, 8274, 8275, 8276, 8277, 8278, 8279, 8280, 8281, 8282, 8283, 8284, 8285, 8286, 8287, 8288, 8289, 8290, 8291, 8292, 8293, 8294, 8295, 8296, 8297, 8298, 8299, 8300, 8301, 8302, 8303, 8304, 8305, 8306, 8307, 8308, 8309, 8310, 8311, 8312, 8313, 8314, 8315, 8316, 8317, 8318, 8319, 8320, 8321, 8322, 8323, 8324, 8325, 8326, 8327, 8328, 8329, 8330, 8331, 8332, 8333, 8334, 8335, 8336, 8337, 8338, 8339, 8340, 8341, 8342, 8343, 8344, 8345, 8346, 8347, 8348, 8349, 8350, 8351, 8352, 8353, 8354, 8355, 8356, 8357, 8358, 8359, 8360, 8361, 8362, 8363, 8364, 8365, 8366, 8367, 8368, 8369, 8370, 8371, 8372, 8373, 8374, 8375, 8376, 8377, 8378, 8379, 8380, 8381, 8382, 8383, 8384, 8385, 8386, 8387, 8388, 8389, 8390, 8391, 8392, 8393, 8394, 8395, 8396, 8397, 8398, 8399, 8400, 8401, 8402, 8403, 8404, 8405, 8406, 8407, 8408, 8409, 8410, 8411, 8412, 8413, 8414, 8415, 8416, 8417, 8418, 8419, 8420, 8421, 8422, 8423, 8424, 8425, 8426, 8427, 8428, 8429, 8430, 8431, 8432, 8433, 8434, 8435, 8436, 8437, 8438, 8439, 8440, 8441, 8442, 8443, 8444, 8445, 8446, 8447, 8448, 8449, 8450, 8451, 8452, 8453, 8454, 8455, 8456, 8457, 8458, 8459, 8460, 8461, 8462, 8463, 8464, 8465, 8466, 8467, 8468, 8469, 8470, 8471, 8472, 8473, 8474, 8475, 8476, 8477, 8478, 8479, 8480, 8481, 8482, 8483, 8484, 8485, 8486, 8487, 8488, 8489, 8490, 8491, 8492, 8493, 8494, 8495, 8496, 8497, 8498, 8499, 8500, 8501, 8502, 8503, 8504, 8505, 8506, 8507, 8508, 8509, 8510, 8511, 8512, 8513, 8514, 8515, 8516, 8517, 8518, 8519, 8520, 8521, 8522, 8523, 8524, 8525, 8526, 8527, 8528, 8529, 8530, 8531, 8532, 8533, 8534, 8535, 8536, 8537, 8538, 8539, 8540, 8541, 8542, 8543, 8544, 8545, 8546, 8547, 8548, 8549, 8550, 8551, 8552, 8553, 8554, 8555, 8556, 8557, 8558, 8559, 8560, 8561, 8562, 8563, 8564, 8565, 8566, 8567, 8568, 8569, 8570, 8571, 8572, 8573, 8574, 8575, 8576, 8577, 8578, 8579, 8580, 8581, 8582, 8583, 8584, 8585, 8586, 8587, 8588, 8589, 8590, 8591, 8592, 8593, 8594, 8595, 8596, 8597, 8598, 8599, 8600, 8601, 8602, 8603, 8604, 8605, 8606, 8607, 8608, 8609, 8610, 8611, 8612, 8613, 8614, 8615, 8616, 8617, 8618, 8619, 8620, 8621, 8622, 8623, 8624, 8625, 8626, 8627, 8628, 8629, 8630, 8631, 8632, 8633, 8634, 8635, 8636, 8637, 8638, 8639, 8640, 8641, 8642, 8643, 8644, 8645, 8646, 8647, 8648, 8649, 8650, 8651, 8652, 8653, 8654, 8655, 8656, 8657, 8658, 8659, 8660, 8661, 8662, 8663, 8664, 8665, 8666, 8667, 8668, 8669, 8670, 8671, 8672, 8673, 8674, 8675, 8676, 8677, 8678, 8679, 8680, 8681, 8682, 8683, 8684, 8685, 8686, 8687, 8688, 8689, 8690, 8691, 8692, 8693, 8694, 8695, 8696, 8697, 8698, 8699, 8700, 8701, 8702, 8703, 8704, 8705, 8706, 8707, 8708, 8709, 8710, 8711, 8712, 8713, 8714, 8715, 8716, 8717, 8718, 8719, 8720, 8721, 8722, 8723, 8724, 8725, 8726, 8727, 8728, 8729, 8730, 8731, 8732, 8733, 8734, 8735, 8736, 8737, 8738, 8739, 8740, 8741, 8742, 8743, 8744, 8745, 8746, 8747, 8748, 8749, 8750, 8751, 8752, 8753, 8754, 8755, 8756, 8757, 8758, 8759, 8760, 8761, 8762, 8763, 8764, 8765, 8766, 8767, 8768, 8769, 8770, 8771, 8772, 8773, 8774, 8775, 8776, 8777, 8778, 8779, 8780, 8781, 8782, 8783, 8784, 8785, 8786, 8787, 8788, 8789, 8790, 8791, 8792, 8793, 8794, 8795, 8796, 8797, 8798, 8799, 8800, 8801, 8802, 8803, 8804, 8805, 8806, 8807, 8808, 8809, 8810, 8811, 8812, 8813, 8814, 8815, 8816, 8817, 8818, 8819, 8820, 8821, 8822, 8823, 8824, 8825, 8826, 8827, 8828, 8829, 8830, 8831, 8832, 8833, 8834, 8835, 8836, 8837, 8838, 8839, 8840, 8841, 8842, 8843, 8844, 8845, 8846, 8847, 8848, 8849, 8850, 8851, 8852, 8853, 8854, 8855, 8856, 8857, 8858, 8859, 8860, 8861, 8862, 8863, 8864, 8865, 8866, 8867, 8868, 8869, 8870, 8871, 8872, 8873, 8874, 8875, 8876, 8877, 8878, 8879, 8880, 8881, 8882, 8883, 8884, 8885, 8886, 8887, 8888, 8889, 8890, 8891, 8892, 8893, 8894, 8895, 8896, 8897, 8898, 8899, 8900, 8901, 8902, 8903, 8904, 8905, 8906, 8907, 8908, 8909, 8910, 8911, 8912, 8913, 8914, 8915, 8916, 8917, 8918, 8919, 8920, 8921, 8922, 8923, 8924, 8925, 8926, 8927, 8928, 8929, 8930, 8931, 8932, 8933, 8934, 8935, 8936, 8937, 8938, 8939, 8940, 8941, 8942, 8943, 8944, 8945, 8946, 8947, 8948, 8949, 8950, 8951, 8952, 8953, 8954, 8955, 8956, 8957, 8958, 8959, 8960, 8961, 8962, 8963, 8964, 8965, 8966, 8967, 8968, 8969, 8970, 8971, 8972, 8973, 8974, 8975, 8976, 8977, 8978, 8979, 8980, 8981, 8982, 8983, 8984, 8985, 8986, 8987, 8988, 8989, 8990, 8991, 8992, 8993, 8994, 8995, 8996, 8997, 8998, 8999, 9000, 9001, 9002, 9003, 9004, 9005, 9006, 9007, 9008, 9009, 9010, 9011, 9012, 9013, 9014, 9015, 9016, 9017, 9018, 9019, 9020, 9021, 9022, 9023, 9024, 9025, 9026, 9027, 9028, 9029, 9030, 9031, 9032, 9033, 9034, 9035, 9036, 9037, 9038, 9039, 9040, 9041, 9042, 9043, 9044, 9045, 9046, 9047, 9048, 9049, 9050, 9051, 9052, 9053, 9054, 9055, 9056, 9057, 9058, 9059, 9060, 9061, 9062, 9063, 9064, 9065, 9066, 9067, 9068, 9069, 9070, 9071, 9072, 9073, 9074, 9075, 9076, 9077, 9078, 9079, 9080, 9081, 9082, 9083, 9084, 9085, 9086, 9087, 9088, 9089, 9090, 9091, 9092, 9093, 9094, 9095, 9096, 9097, 9098, 9099, 9100, 9101, 9102, 9103, 9104, 9105, 9106, 9107, 9108, 9109, 9110, 9111, 9112, 9113, 9114, 9115, 9116, 9117, 9118, 9119, 9120, 9121, 9122, 9123, 9124, 9125, 9126, 9127, 9128, 9129, 9130, 9131, 9132, 9133, 9134, 9135, 9136, 9137, 9138, 9139, 9140, 9141, 9142, 9143, 9144, 9145, 9146, 9147, 9148, 9149, 9150, 9151, 9152, 9153, 9154, 9155, 9156, 9157, 9158, 9159, 9160, 9161, 9162, 9163, 9164, 9165, 9166, 9167, 9168, 9169, 9170, 9171, 9172, 9173, 9174, 9175, 9176, 9177, 9178, 9179, 9180, 9181, 9182, 9183, 9184, 9185, 9186, 9187, 9188, 9189, 9190, 9191, 9192, 9193, 9194, 9195, 9196, 9197, 9198, 9199, 9200, 9201, 9202, 9203, 9204, 9205, 9206, 9207, 9208, 9209, 9210, 9211, 9212, 9213, 9214, 9215, 9216, 9217, 9218, 9219, 9220, 9221, 9222, 9223, 9224, 9225, 9226, 9227, 9228, 9229, 9230, 9231, 9232, 9233, 9234, 9235, 9236, 9237, 9238, 9239, 9240, 9241, 9242, 9243, 9244, 9245, 9246, 9247, 9248, 9249, 9250, 9251, 9252, 9253, 9254, 9255, 9256, 9257, 9258, 9259, 9260, 9261, 9262, 9263, 9264, 9265, 9266, 9267, 9268, 9269, 9270, 9271, 9272, 9273, 9274, 9275, 9276, 9277, 9278, 9279, 9280, 9281, 9282, 9283, 9284, 9285, 9286, 9287, 9288, 9289, 9290, 9291, 9292, 9293, 9294, 9295, 9296, 9297, 9298, 9299, 9300, 9301, 9302, 9303, 9304, 9305, 9306, 9307, 9308, 9309, 9310, 9311, 9312, 9313, 9314, 9315, 9316, 9317, 9318, 9319, 9320, 9321, 9322, 9323, 9324, 9325, 9326, 9327, 9328, 9329, 9330, 9331, 9332, 9333, 9334, 9335, 9336, 9337, 9338, 9339, 9340, 9341, 9342, 9343, 9344, 9345, 9346, 9347, 9348, 9349, 9350, 9351, 9352, 9353, 9354, 9355, 9356, 9357, 9358, 9359, 9360, 9361, 9362, 9363, 9364, 9365, 9366, 9367, 9368, 9369, 9370, 9371, 9372, 9373, 9374, 9375, 9376, 9377, 9378, 9379, 9380, 9381, 9382, 9383, 9384, 9385, 9386, 9387, 9388, 9389, 9390, 9391, 9392, 9393, 9394, 9395, 9396, 9397, 9398, 9399, 9400, 9401, 9402, 9403, 9404, 9405, 9406, 9407, 9408, 9409, 9410, 9411, 9412, 9413, 9414, 9415, 9416, 9417, 9418, 9419, 9420, 9421, 9422, 9423, 9424, 9425, 9426, 9427, 9428, 9429, 9430, 9431, 9432, 9433, 9434, 9435, 9436, 9437, 9438, 9439, 9440, 9441, 9442, 9443, 9444, 9445, 9446, 9447, 9448, 9449, 9450, 9451, 9452, 9453, 9454, 9455, 9456, 9457, 9458, 9459, 9460, 9461, 9462, 9463, 9464, 9465, 9466, 9467, 9468, 9469, 9470, 9471, 9472, 9473, 9474, 9475, 9476, 9477, 9478, 9479, 9480, 9481, 9482, 9483, 9484, 9485, 9486, 9487, 9488, 9489, 9490, 9491, 9492, 9493, 9494, 9495, 9496, 9497, 9498, 9499, 9500, 9501, 9502, 9503, 9504, 9505, 9506, 9507, 9508, 9509, 9510, 9511, 9512, 9513, 9514, 9515, 9516, 9517, 9518, 9519, 9520, 9521, 9522, 9523, 9524, 9525, 9526, 9527, 9528, 9529, 9530, 9531, 9532, 9533, 9534, 9535, 9536, 9537, 9538, 9539, 9540, 9541, 9542, 9543, 9544, 9545, 9546, 9547, 9548, 9549, 9550, 9551, 9552, 9553, 9554, 9555, 9556, 9557, 9558, 9559, 9560, 9561, 9562, 9563, 9564, 9565, 9566, 9567, 9568, 9569, 9570, 9571, 9572, 9573, 9574, 9575, 9576, 9577, 9578, 9579, 9580, 9581, 9582, 9583, 9584, 9585, 9586, 9587, 9588, 9589, 9590, 9591, 9592, 9593, 9594, 9595, 9596, 9597, 9598, 9599, 9600, 9601, 9602, 9603, 9604, 9605, 9606, 9607, 9608, 9609, 9610, 9611, 9612, 9613, 9614, 9615, 9616, 9617, 9618, 9619, 9620, 9621, 9622, 9623, 9624, 9625, 9626, 9627, 9628, 9629, 9630, 9631, 9632, 9633, 9634, 9635, 9636, 9637, 9638, 9639, 9640, 9641, 9642, 9643, 9644, 9645, 9646, 9647, 9648, 9649, 9650, 9651, 9652, 9653, 9654, 9655, 9656, 9657, 9658, 9659, 9660, 9661, 9662, 9663, 9664, 9665, 9666, 9667, 9668, 9669, 9670, 9671, 9672, 9673, 9674, 9675, 9676, 9677, 9678, 9679, 9680, 9681, 9682, 9683, 9684, 9685, 9686, 9687, 9688, 9689, 9690, 9691, 9692, 9693, 9694, 9695, 9696, 9697, 9698, 9699, 9700, 9701, 9702, 9703, 9704, 9705, 9706, 9707, 9708, 9709, 9710, 9711, 9712, 9713, 9714, 9715, 9716, 9717, 9718, 9719, 9720, 9721, 9722, 9723, 9724, 9725, 9726, 9727, 9728, 9729, 9730, 9731, 9732, 9733, 9734, 9735, 9736, 9737, 9738, 9739, 9740, 9741, 9742, 9743, 9744, 9745, 9746, 9747, 9748, 9749, 9750, 9751, 9752, 9753, 9754, 9755, 9756, 9757, 9758, 9759, 9760, 9761, 9762, 9763, 9764, 9765, 9766, 9767, 9768, 9769, 9770, 9771, 9772, 9773, 9774, 9775, 9776, 9777, 9778, 9779, 9780, 9781, 9782, 9783, 9784, 9785, 9786, 9787, 9788, 9789, 9790, 9791, 9792, 9793, 9794, 9795, 9796, 9797, 9798, 9799, 9800, 9801, 9802, 9803, 9804, 9805, 9806, 9807, 9808, 9809, 9810, 9811, 9812, 9813, 9814, 9815, 9816, 9817, 9818, 9819, 9820, 9821, 9822, 9823, 9824, 9825, 9826, 9827, 9828, 9829, 9830, 9831, 9832, 9833, 9834, 9835, 9836, 9837, 9838, 9839, 9840, 9841, 9842, 9843, 9844, 9845, 9846, 9847, 9848, 9849, 9850, 9851, 9852, 9853, 9854, 9855, 9856, 9857, 9858, 9859, 9860, 9861, 9862, 9863, 9864, 9865, 9866, 9867, 9868, 9869, 9870, 9871, 9872, 9873, 9874, 9875, 9876, 9877, 9878, 9879, 9880, 9881, 9882, 9883, 9884, 9885, 9886, 9887, 9888, 9889, 9890, 9891, 9892, 9893, 9894, 9895, 9896, 9897, 9898, 9899, 9900, 9901, 9902, 9903, 9904, 9905, 9906, 9907, 9908, 9909, 9910, 9911, 9912, 9913, 9914, 9915, 9916, 9917, 9918, 9919, 9920, 9921, 9922, 9923, 9924, 9925, 9926, 9927, 9928, 9929, 9930, 9931, 9932, 9933, 9934, 9935, 9936, 9937, 9938, 9939, 9940, 9941, 9942, 9943, 9944, 9945, 9946, 9947, 9948, 9949, 9950, 9951, 9952, 9953, 9954, 9955, 9956, 9957, 9958, 9959, 9960, 9961, 9962, 9963, 9964, 9965, 9966, 9967, 9968, 9969, 9970, 9971, 9972, 9973, 9974, 9975, 9976, 9977, 9978, 9979, 9980, 9981, 9982, 9983, 9984, 9985, 9986, 9987, 9988, 9989, 9990, 9991, 9992, 9993, 9994, 9995, 9996, 9997, 9998, 9999, 10000, 10001, 10002, 10003, 10004, 10005, 10006, 10007, 10008, 10009, 10010, 10011, 10012, 10013, 10014, 10015, 10016, 10017, 10018, 10019, 10020, 10021, 10022, 10023, 10024, 10025, 10026, 10027, 10028, 10029, 10030, 10031, 10032, 10033, 10034, 10035, 10036, 10037, 10038, 10039, 10040, 10041, 10042, 10043, 10044, 10045, 10046, 10047, 10048, 10049, 10050, 10051, 10052, 10053, 10054, 10055, 10056, 10057, 10058, 10059, 10060, 10061, 10062, 10063, 10064, 10065, 10066, 10067, 10068, 10069, 10070, 10071, 10072, 10073, 10074, 10075, 10076, 10077, 10078, 10079, 10080, 10081, 10082, 10083, 10084, 10085, 10086, 10087, 10088, 10089, 10090, 10091, 10092, 10093, 10094, 10095, 10096, 10097, 10098, 10099, 10100, 10101, 10102, 10103, 10104, 10105, 10106, 10107, 10108, 10109, 10110, 10111, 10112, 10113, 10114, 10115, 10116, 10117, 10118, 10119, 10120, 10121, 10122, 10123, 10124, 10125, 10126, 10127, 10128, 10129, 10130, 10131, 10132, 10133, 10134, 10135, 10136, 10137, 10138, 10139, 10140, 10141, 10142, 10143, 10144, 10145, 10146, 10147, 10148, 10149, 10150, 10151, 10152, 10153, 10154, 10155, 10156, 10157, 10158, 10159, 10160, 10161, 10162, 10163, 10164, 10165, 10166, 10167, 10168, 10169, 10170, 10171, 10172, 10173, 10174, 10175, 10176, 10177, 10178, 10179, 10180, 10181, 10182, 10183, 10184, 10185, 10186, 10187, 10188, 10189, 10190, 10191, 10192, 10193, 10194, 10195, 10196, 10197, 10198, 10199, 10200, 10201, 10202, 10203, 10204, 10205, 10206, 10207, 10208, 10209, 10210, 10211, 10212, 10213, 10214, 10215, 10216, 10217, 10218, 10219, 10220, 10221, 10222, 10223, 10224, 10225, 10226, 10227, 10228, 10229, 10230, 10231, 10232, 10233, 10234, 10235, 10236, 10237, 10238, 10239, 10240, 10241, 10242, 10243, 10244, 10245, 10246, 10247, 10248, 10249, 10250, 10251, 10252, 10253, 10254, 10255, 10256, 10257, 10258, 10259, 10260, 10261, 10262, 10263, 10264, 10265, 10266, 10267, 10268, 10269, 10270, 10271, 10272, 10273, 10274, 10275, 10276, 10277, 10278, 10279, 10280, 10281, 10282, 10283, 10284, 10285, 10286, 10287, 10288, 10289, 10290, 10291, 10292, 10293, 10294, 10295, 10296, 10297, 10298, 10299, 10300, 10301, 10302, 10303, 10304, 10305, 10306, 10307, 10308, 10309, 10310, 10311, 10312, 10313, 10314, 10315, 10316, 10317, 10318, 10319, 10320, 10321, 10322, 10323, 10324, 10325, 10326, 10327, 10328, 10329, 10330, 10331, 10332, 10333, 10334, 10335, 10336, 10337, 10338, 10339, 10340, 10341, 10342, 10343, 10344, 10345, 10346, 10347, 10348, 10349, 10350, 10351, 10352, 10353, 10354, 10355, 10356, 10357, 10358, 10359, 10360, 10361, 10362, 10363, 10364, 10365, 10366, 10367, 10368, 10369, 10370, 10371, 10372, 10373, 10374, 10375, 10376, 10377, 10378, 10379, 10380, 10381, 10382, 10383, 10384, 10385, 10386, 10387, 10388, 10389, 10390, 10391, 10392, 10393, 10394, 10395, 10396, 10397, 10398, 10399, 10400, 10401, 10402, 10403, 10404, 10405, 10406, 10407, 10408, 10409, 10410, 10411, 10412, 10413, 10414, 10415, 10416, 10417, 10418, 10419, 10420, 10421, 10422, 10423, 10424, 10425, 10426, 10427, 10428, 10429, 10430, 10431, 10432, 10433, 10434, 10435, 10436, 10437, 10438, 10439, 10440, 10441, 10442, 10443, 10444, 10445, 10446, 10447, 10448, 10449, 10450, 10451, 10452, 10453, 10454, 10455, 10456, 10457, 10458, 10459, 10460, 10461, 10462, 10463, 10464, 10465, 10466, 10467, 10468, 10469, 10470, 10471, 10472, 10473, 10474, 10475, 10476, 10477, 10478, 10479, 10480, 10481, 10482, 10483, 10484, 10485, 10486, 10487, 10488, 10489, 10490, 10491, 10492, 10493, 10494, 10495, 10496, 10497, 10498, 10499, 10500, 10501, 10502, 10503, 10504, 10505, 10506, 10507, 10508, 10509, 10510, 10511, 10512, 10513, 10514, 10515, 10516, 10517, 10518, 10519, 10520, 10521, 10522, 10523, 10524, 10525, 10526, 10527, 10528, 10529, 10530, 10531, 10532, 10533, 10534, 10535, 10536, 10537, 10538, 10539, 10540, 10541, 10542, 10543, 10544, 10545, 10546, 10547, 10548, 10549, 10550, 10551, 10552, 10553, 10554, 10555, 10556, 10557, 10558, 10559, 10560, 10561, 10562, 10563, 10564, 10565, 10566, 10567, 10568, 10569, 10570, 10571, 10572, 10573, 10574, 10575, 10576, 10577, 10578, 10579, 10580, 10581, 10582, 10583, 10584, 10585, 10586, 10587, 10588, 10589, 10590, 10591, 10592, 10593, 10594, 10595, 10596, 10597, 10598, 10599, 10600, 10601, 10602, 10603, 10604, 10605, 10606, 10607, 10608, 10609, 10610, 10611, 10612, 10613, 10614, 10615, 10616, 10617, 10618, 10619, 10620, 10621, 10622, 10623, 10624, 10625, 10626, 10627, 10628, 10629, 10630, 10631, 10632, 10633, 10634, 10635, 10636, 10637, 10638, 10639, 10640, 10641, 10642, 10643, 10644, 10645, 10646, 10647, 10648, 10649, 10650, 10651, 10652, 10653, 10654, 10655, 10656, 10657, 10658, 10659, 10660, 10661, 10662, 10663, 10664, 10665, 10666, 10667, 10668, 10669, 10670, 10671, 10672, 10673, 10674, 10675, 10676, 10677, 10678, 10679, 10680, 10681, 10682, 10683, 10684, 10685, 10686, 10687, 10688, 10689, 10690, 10691, 10692, 10693, 10694, 10695, 10696, 10697, 10698, 10699, 10700, 10701, 10702, 10703, 10704, 10705, 10706, 10707, 10708, 10709, 10710, 10711, 10712, 10713, 10714, 10715, 10716, 10717, 10718, 10719, 10720, 10721, 10722, 10723, 10724, 10725, 10726, 10727, 10728, 10729, 10730, 10731, 10732, 10733, 10734, 10735, 10736, 10737, 10738, 10739, 10740, 10741, 10742, 10743, 10744, 10745, 10746, 10747, 10748, 10749, 10750, 10751, 10752, 10753, 10754, 10755, 10756, 10757, 10758, 10759, 10760, 10761, 10762, 10763, 10764, 10765, 10766, 10767, 10768, 10769, 10770, 10771, 10772, 10773, 10774, 10775, 10776, 10777, 10778, 10779, 10780, 10781, 10782, 10783, 10784, 10785, 10786, 10787, 10788, 10789, 10790, 10791, 10792, 10793, 10794, 10795, 10796, 10797, 10798, 10799, 10800, 10801, 10802, 10803, 10804, 10805, 10806, 10807, 10808, 10809, 10810, 10811, 10812, 10813, 10814, 10815, 10816, 10817, 10818, 10819, 10820, 10821, 10822, 10823, 10824, 10825, 10826, 10827, 10828, 10829, 10830, 10831, 10832, 10833, 10834, 10835, 10836, 10837, 10838, 10839, 10840, 10841, 10842, 10843, 10844, 10845, 10846, 10847, 10848, 10849, 10850, 10851, 10852, 10853, 10854, 10855, 10856, 10857, 10858, 10859, 10860, 10861, 10862, 10863, 10864, 10865, 10866, 10867, 10868, 10869, 10870, 10871, 10872, 10873, 10874, 10875, 10876, 10877, 10878, 10879, 10880, 10881, 10882, 10883, 10884, 10885, 10886, 10887, 10888, 10889, 10890, 10891, 10892, 10893, 10894, 10895, 10896, 10897, 10898, 10899, 10900, 10901, 10902, 10903, 10904, 10905, 10906, 10907, 10908, 10909, 10910, 10911, 10912, 10913, 10914, 10915, 10916, 10917, 10918, 10919, 10920, 10921, 10922, 10923, 10924, 10925, 10926, 10927, 10928, 10929, 10930, 10931, 10932, 10933, 10934, 10935, 10936, 10937, 10938, 10939, 10940, 10941, 10942, 10943, 10944, 10945, 10946, 10947, 10948, 10949, 10950, 10951, 10952, 10953, 10954, 10955, 10956, 10957, 10958, 10959, 10960, 10961, 10962, 10963, 10964, 10965, 10966, 10967, 10968, 10969, 10970, 10971, 10972, 10973, 10974, 10975, 10976, 10977, 10978, 10979, 10980, 10981, 10982, 10983, 10984, 10985, 10986, 10987, 10988, 10989, 10990, 10991, 10992, 10993, 10994, 10995, 10996, 10997, 10998, 10999, 11000, 11001, 11002, 11003, 11004, 11005, 11006, 11007, 11008, 11009, 11010, 11011, 11012, 11013, 11014, 11015, 11016, 11017, 11018, 11019, 11020, 11021, 11022, 11023, 11024, 11025, 11026, 11027, 11028, 11029, 11030, 11031, 11032, 11033, 11034, 11035, 11036, 11037, 11038, 11039, 11040, 11041, 11042, 11043, 11044, 11045, 11046, 11047, 11048, 11049, 11050, 11051, 11052, 11053, 11054, 11055, 11056, 11057, 11058, 11059, 11060, 11061, 11062, 11063, 11064, 11065, 11066, 11067, 11068, 11069, 11070, 11071, 11072, 11073, 11074, 11075, 11076, 11077, 11078, 11079, 11080, 11081, 11082, 11083, 11084, 11085, 11086, 11087, 11088, 11089, 11090, 11091, 11092, 11093, 11094, 11095, 11096, 11097, 11098, 11099, 11100, 11101, 11102, 11103, 11104, 11105, 11106, 11107, 11108, 11109, 11110, 11111, 11112, 11113, 11114, 11115, 11116, 11117, 11118, 11119, 11120, 11121, 11122, 11123, 11124, 11125, 11126, 11127, 11128, 11129, 11130, 11131, 11132, 11133, 11134, 11135, 11136, 11137, 11138, 11139, 11140, 11141, 11142, 11143, 11144, 11145, 11146, 11147, 11148, 11149, 11150, 11151, 11152, 11153, 11154, 11155, 11156, 11157, 11158, 11159, 11160, 11161, 11162, 11163, 11164, 11165, 11166, 11167, 11168, 11169, 11170, 11171, 11172, 11173, 11174, 11175, 11176, 11177, 11178, 11179, 11180, 11181, 11182, 11183, 11184, 11185, 11186, 11187, 11188, 11189, 11190, 11191, 11192, 11193, 11194, 11195, 11196, 11197, 11198, 11199, 11200, 11201, 11202, 11203, 11204, 11205, 11206, 11207, 11208, 11209, 11210, 11211, 11212, 11213, 11214, 11215, 11216, 11217, 11218, 11219, 11220, 11221, 11222, 11223, 11224, 11225, 11226, 11227, 11228, 11229, 11230, 11231, 11232, 11233, 11234, 11235, 11236, 11237, 11238, 11239, 11240, 11241, 11242, 11243, 11244, 11245, 11246, 11247, 11248, 11249, 11250, 11251, 11252, 11253, 11254, 11255, 11256, 11257, 11258, 11259, 11260, 11261, 11262, 11263, 11264, 11265, 11266, 11267, 11268, 11269, 11270, 11271, 11272, 11273, 11274, 11275, 11276, 11277, 11278, 11279, 11280, 11281, 11282, 11283, 11284, 11285, 11286, 11287, 11288, 11289, 11290, 11291, 11292, 11293, 11294, 11295, 11296, 11297, 11298, 11299, 11300, 11301, 11302, 11303, 11304, 11305, 11306, 11307, 11308, 11309, 11310, 11311, 11312, 11313, 11314, 11315, 11316, 11317, 11318, 11319, 11320, 11321, 11322, 11323, 11324, 11325, 11326, 11327, 11328, 11329, 11330, 11331, 11332, 11333, 11334, 11335, 11336, 11337, 11338, 11339, 11340, 11341, 11342, 11343, 11344, 11345, 11346, 11347, 11348, 11349, 11350, 11351, 11352, 11353, 11354, 11355, 11356, 11357, 11358, 11359, 11360, 11361, 11362, 11363, 11364, 11365, 11366, 11367, 11368, 11369, 11370, 11371, 11372, 11373, 11374, 11375, 11376, 11377, 11378, 11379, 11380, 11381, 11382, 11383, 11384, 11385, 11386, 11387, 11388, 11389, 11390, 11391, 11392, 11393, 11394, 11395, 11396, 11397, 11398, 11399, 11400, 11401, 11402, 11403, 11404, 11405, 11406, 11407, 11408, 11409, 11410, 11411, 11412, 11413, 11414, 11415, 11416, 11417, 11418, 11419, 11420, 11421, 11422, 11423, 11424, 11425, 11426, 11427, 11428, 11429, 11430, 11431, 11432, 11433, 11434, 11435, 11436, 11437, 11438, 11439, 11440, 11441, 11442, 11443, 11444, 11445, 11446, 11447, 11448, 11449, 11450, 11451, 11452, 11453, 11454, 11455, 11456, 11457, 11458, 11459, 11460, 11461, 11462, 11463, 11464, 11465, 11466, 11467, 11468, 11469, 11470, 11471, 11472, 11473, 11474, 11475, 11476, 11477, 11478, 11479, 11480, 11481, 11482, 11483, 11484, 11485, 11486, 11487, 11488, 11489, 11490, 11491, 11492, 11493, 11494, 11495, 11496, 11497, 11498, 11499, 11500, 11501, 11502, 11503, 11504, 11505, 11506, 11507, 11508, 11509, 11510, 11511, 11512, 11513, 11514, 11515, 11516, 11517, 11518, 11519, 11520, 11521, 11522, 11523, 11524, 11525, 11526, 11527, 11528, 11529, 11530, 11531, 11532, 11533, 11534, 11535, 11536, 11537, 11538, 11539, 11540, 11541, 11542, 11543, 11544, 11545, 11546, 11547, 11548, 11549, 11550, 11551, 11552, 11553, 11554, 11555, 11556, 11557, 11558, 11559, 11560, 11561, 11562, 11563, 11564, 11565, 11566, 11567, 11568, 11569, 11570, 11571, 11572, 11573, 11574, 11575, 11576, 11577, 11578, 11579, 11580, 11581, 11582, 11583, 11584, 11585, 11586, 11587, 11588, 11589, 11590, 11591, 11592, 11593, 11594, 11595, 11596, 11597, 11598, 11599, 11600, 11601, 11602, 11603, 11604, 11605, 11606, 11607, 11608, 11609, 11610, 11611, 11612, 11613, 11614, 11615, 11616, 11617, 11618, 11619, 11620, 11621, 11622, 11623, 11624, 11625, 11626, 11627, 11628, 11629, 11630, 11631, 11632, 11633, 11634, 11635, 11636, 11637, 11638, 11639, 11640, 11641, 11642, 11643, 11644, 11645, 11646, 11647, 11648, 11649, 11650, 11651, 11652, 11653, 11654, 11655, 11656, 11657, 11658, 11659, 11660, 11661, 11662, 11663, 11664, 11665, 11666, 11667, 11668, 11669, 11670, 11671, 11672, 11673, 11674, 11675, 11676, 11677, 11678, 11679, 11680, 11681, 11682, 11683, 11684, 11685, 11686, 11687, 11688, 11689, 11690, 11691, 11692, 11693, 11694, 11695, 11696, 11697, 11698, 11699, 11700, 11701, 11702, 11703, 11704, 11705, 11706, 11707, 11708, 11709, 11710, 11711, 11712, 11713, 11714, 11715, 11716, 11717, 11718, 11719, 11720, 11721, 11722, 11723, 11724, 11725, 11726, 11727, 11728, 11729, 11730, 11731, 11732, 11733, 11734, 11735, 11736, 11737, 11738, 11739, 11740, 11741, 11742, 11743, 11744, 11745, 11746, 11747, 11748, 11749, 11750, 11751, 11752, 11753, 11754, 11755, 11756, 11757, 11758, 11759, 11760, 11761, 11762, 11763, 11764, 11765, 11766, 11767, 11768, 11769, 11770, 11771, 11772, 11773, 11774, 11775, 11776, 11777, 11778, 11779, 11780, 11781, 11782, 11783, 11784, 11785, 11786, 11787, 11788, 11789, 11790, 11791, 11792, 11793, 11794, 11795, 11796, 11797, 11798, 11799, 11800, 11801, 11802, 11803, 11804, 11805, 11806, 11807, 11808, 11809, 11810, 11811, 11812, 11813, 11814, 11815, 11816, 11817, 11818, 11819, 11820, 11821, 11822, 11823, 11824, 11825, 11826, 11827, 11828, 11829, 11830, 11831, 11832, 11833, 11834, 11835, 11836, 11837, 11838, 11839, 11840, 11841, 11842, 11843, 11844, 11845, 11846, 11847, 11848, 11849, 11850, 11851, 11852, 11853, 11854, 11855, 11856, 11857, 11858, 11859, 11860, 11861, 11862, 11863, 11864, 11865, 11866, 11867, 11868, 11869, 11870, 11871, 11872, 11873, 11874, 11875, 11876, 11877, 11878, 11879, 11880, 11881, 11882, 11883, 11884, 11885, 11886, 11887, 11888, 11889, 11890, 11891, 11892, 11893, 11894, 11895, 11896, 11897, 11898, 11899, 11900, 11901, 11902, 11903, 11904, 11905, 11906, 11907, 11908, 11909, 11910, 11911, 11912, 11913, 11914, 11915, 11916, 11917, 11918, 11919, 11920, 11921, 11922, 11923, 11924, 11925, 11926, 11927, 11928, 11929, 11930, 11931, 11932, 11933, 11934, 11935, 11936, 11937, 11938, 11939, 11940, 11941, 11942, 11943, 11944, 11945, 11946, 11947, 11948, 11949, 11950, 11951, 11952, 11953, 11954, 11955, 11956, 11957, 11958, 11959, 11960, 11961, 11962, 11963, 11964, 11965, 11966, 11967, 11968, 11969, 11970, 11971, 11972, 11973, 11974, 11975, 11976, 11977, 11978, 11979, 11980, 11981, 11982, 11983, 11984, 11985, 11986, 11987, 11988, 11989, 11990, 11991, 11992, 11993, 11994, 11995, 11996, 11997, 11998, 11999, 12000, 12001, 12002, 12003, 12004, 12005, 12006, 12007, 12008, 12009, 12010, 12011, 12012, 12013, 12014, 12015, 12016, 12017, 12018, 12019, 12020, 12021, 12022, 12023, 12024, 12025, 12026, 12027, 12028, 12029, 12030, 12031, 12032, 12033, 12034, 12035, 12036, 12037, 12038, 12039, 12040, 12041, 12042, 12043, 12044, 12045, 12046, 12047, 12048, 12049, 12050, 12051, 12052, 12053, 12054, 12055, 12056, 12057, 12058, 12059, 12060, 12061, 12062, 12063, 12064, 12065, 12066, 12067, 12068, 12069, 12070, 12071, 12072, 12073, 12074, 12075, 12076, 12077, 12078, 12079, 12080, 12081, 12082, 12083, 12084, 12085, 12086, 12087, 12088, 12089, 12090, 12091, 12092, 12093, 12094, 12095, 12096, 12097, 12098, 12099, 12100, 12101, 12102, 12103, 12104, 12105, 12106, 12107, 12108, 12109, 12110, 12111, 12112, 12113, 12114, 12115, 12116, 12117, 12118, 12119, 12120, 12121, 12122, 12123, 12124, 12125, 12126, 12127, 12128, 12129, 12130, 12131, 12132, 12133, 12134, 12135, 12136, 12137, 12138, 12139, 12140, 12141, 12142, 12143, 12144, 12145, 12146, 12147, 12148, 12149, 12150, 12151, 12152, 12153, 12154, 12155, 12156, 12157, 12158, 12159, 12160, 12161, 12162, 12163, 12164, 12165, 12166, 12167, 12168, 12169, 12170, 12171, 12172, 12173, 12174, 12175, 12176, 12177, 12178, 12179, 12180, 12181, 12182, 12183, 12184, 12185, 12186, 12187, 12188, 12189, 12190, 12191, 12192, 12193, 12194, 12195, 12196, 12197, 12198, 12199, 12200, 12201, 12202, 12203, 12204, 12205, 12206, 12207, 12208, 12209, 12210, 12211, 12212, 12213, 12214, 12215, 12216, 12217, 12218, 12219, 12220, 12221, 12222, 12223, 12224, 12225, 12226, 12227, 12228, 12229, 12230, 12231, 12232, 12233, 12234, 12235, 12236, 12237, 12238, 12239, 12240, 12241, 12242, 12243, 12244, 12245, 12246, 12247, 12248, 12249, 12250, 12251, 12252, 12253, 12254, 12255, 12256, 12257, 12258, 12259, 12260, 12261, 12262, 12263, 12264, 12265, 12266, 12267, 12268, 12269, 12270, 12271, 12272, 12273, 12274, 12275, 12276, 12277, 12278, 12279, 12280, 12281, 12282, 12283, 12284, 12285, 12286, 12287, 12288, 12289, 12290, 12291, 12292, 12293, 12294, 12295, 12296, 12297, 12298, 12299, 12300, 12301, 12302, 12303, 12304, 12305, 12306, 12307, 12308, 12309, 12310, 12311, 12312, 12313, 12314, 12315, 12316, 12317, 12318, 12319, 12320, 12321, 12322, 12323, 12324, 12325, 12326, 12327, 12328, 12329, 12330, 12331, 12332, 12333, 12334, 12335, 12336, 12337, 12338, 12339, 12340, 12341, 12342, 12343, 12344, 12345, 12346, 12347, 12348, 12349, 12350, 12351, 12352, 12353, 12354, 12355, 12356, 12357, 12358, 12359, 12360, 12361, 12362, 12363, 12364, 12365, 12366, 12367, 12368, 12369, 12370, 12371, 12372, 12373, 12374, 12375, 12376, 12377, 12378, 12379, 12380, 12381, 12382, 12383, 12384, 12385, 12386, 12387, 12388, 12389, 12390, 12391, 12392, 12393, 12394, 12395, 12396, 12397, 12398, 12399, 12400, 12401, 12402, 12403, 12404, 12405, 12406, 12407, 12408, 12409, 12410, 12411, 12412, 12413, 12414, 12415, 12416, 12417, 12418, 12419, 12420, 12421, 12422, 12423, 12424, 12425, 12426, 12427, 12428, 12429, 12430, 12431, 12432, 12433, 12434, 12435, 12436, 12437, 12438, 12439, 12440, 12441, 12442, 12443, 12444, 12445, 12446, 12447, 12448, 12449, 12450, 12451, 12452, 12453, 12454, 12455, 12456, 12457, 12458, 12459, 12460, 12461, 12462, 12463, 12464, 12465, 12466, 12467, 12468, 12469, 12470, 12471, 12472, 12473, 12474, 12475, 12476, 12477, 12478, 12479, 12480, 12481, 12482, 12483, 12484, 12485, 12486, 12487, 12488, 12489, 12490, 12491, 12492, 12493, 12494, 12495, 12496, 12497, 12498, 12499, 12500, 12501, 12502, 12503, 12504, 12505, 12506, 12507, 12508, 12509, 12510, 12511, 12512, 12513, 12514, 12515, 12516, 12517, 12518, 12519, 12520, 12521, 12522, 12523, 12524, 12525, 12526, 12527, 12528, 12529, 12530, 12531, 12532, 12533, 12534, 12535, 12536, 12537, 12538, 12539, 12540, 12541, 12542, 12543, 12544, 12545, 12546, 12547, 12548, 12549, 12550, 12551, 12552, 12553, 12554, 12555, 12556, 12557, 12558, 12559, 12560, 12561, 12562, 12563, 12564, 12565, 12566, 12567, 12568, 12569, 12570, 12571, 12572, 12573, 12574, 12575, 12576, 12577, 12578, 12579, 12580, 12581, 12582, 12583, 12584, 12585, 12586, 12587, 12588, 12589, 12590, 12591, 12592, 12593, 12594, 12595, 12596, 12597, 12598, 12599, 12600, 12601, 12602, 12603, 12604, 12605, 12606, 12607, 12608, 12609, 12610, 12611, 12612, 12613, 12614, 12615, 12616, 12617, 12618, 12619, 12620, 12621, 12622, 12623, 12624, 12625, 12626, 12627, 12628, 12629, 12630, 12631, 12632, 12633, 12634, 12635, 12636, 12637, 12638, 12639, 12640, 12641, 12642, 12643, 12644, 12645, 12646, 12647, 12648, 12649, 12650, 12651, 12652, 12653, 12654, 12655, 12656, 12657, 12658, 12659, 12660, 12661, 12662, 12663, 12664, 12665, 12666, 12667, 12668, 12669, 12670, 12671, 12672, 12673, 12674, 12675, 12676, 12677, 12678, 12679, 12680, 12681, 12682, 12683, 12684, 12685, 12686, 12687, 12688, 12689, 12690, 12691, 12692, 12693, 12694, 12695, 12696, 12697, 12698, 12699, 12700, 12701, 12702, 12703, 12704, 12705, 12706, 12707, 12708, 12709, 12710, 12711, 12712, 12713, 12714, 12715, 12716, 12717, 12718, 12719, 12720, 12721, 12722, 12723, 12724, 12725, 12726, 12727, 12728, 12729, 12730, 12731, 12732, 12733, 12734, 12735, 12736, 12737, 12738, 12739, 12740, 12741, 12742, 12743, 12744, 12745, 12746, 12747, 12748, 12749, 12750, 12751, 12752, 12753, 12754, 12755, 12756, 12757, 12758, 12759, 12760, 12761, 12762, 12763, 12764, 12765, 12766, 12767, 12768, 12769, 12770, 12771, 12772, 12773, 12774, 12775, 12776, 12777, 12778, 12779, 12780, 12781, 12782, 12783, 12784, 12785, 12786, 12787, 12788, 12789, 12790, 12791, 12792, 12793, 12794, 12795, 12796, 12797, 12798, 12799, 12800, 12801, 12802, 12803, 12804, 12805, 12806, 12807, 12808, 12809, 12810, 12811, 12812, 12813, 12814, 12815, 12816, 12817, 12818, 12819, 12820, 12821, 12822, 12823, 12824, 12825, 12826, 12827, 12828, 12829, 12830, 12831, 12832, 12833, 12834, 12835, 12836, 12837, 12838, 12839, 12840, 12841, 12842, 12843, 12844, 12845, 12846, 12847, 12848, 12849, 12850, 12851, 12852, 12853, 12854, 12855, 12856, 12857, 12858, 12859, 12860, 12861, 12862, 12863, 12864, 12865, 12866, 12867, 12868, 12869, 12870, 12871, 12872, 12873, 12874, 12875, 12876, 12877, 12878, 12879, 12880, 12881, 12882, 12883, 12884, 12885, 12886, 12887, 12888, 12889, 12890, 12891, 12892, 12893, 12894, 12895, 12896, 12897, 12898, 12899, 12900, 12901, 12902, 12903, 12904, 12905, 12906, 12907, 12908, 12909, 12910, 12911, 12912, 12913, 12914, 12915, 12916, 12917, 12918, 12919, 12920, 12921, 12922, 12923, 12924, 12925, 12926, 12927, 12928, 12929, 12930, 12931, 12932, 12933, 12934, 12935, 12936, 12937, 12938, 12939, 12940, 12941, 12942, 12943, 12944, 12945, 12946, 12947, 12948, 12949, 12950, 12951, 12952, 12953, 12954, 12955, 12956, 12957, 12958, 12959, 12960, 12961, 12962, 12963, 12964, 12965, 12966, 12967, 12968, 12969, 12970, 12971, 12972, 12973, 12974, 12975, 12976, 12977, 12978, 12979, 12980, 12981, 12982, 12983, 12984, 12985, 12986, 12987, 12988, 12989, 12990, 12991, 12992, 12993, 12994, 12995, 12996, 12997, 12998, 12999, 13000, 13001, 13002, 13003, 13004, 13005, 13006, 13007, 13008, 13009, 13010, 13011, 13012, 13013, 13014, 13015, 13016, 13017, 13018, 13019, 13020, 13021, 13022, 13023, 13024, 13025, 13026, 13027, 13028, 13029, 13030, 13031, 13032, 13033, 13034, 13035, 13036, 13037, 13038, 13039, 13040, 13041, 13042, 13043, 13044, 13045, 13046, 13047, 13048, 13049, 13050, 13051, 13052, 13053, 13054, 13055, 13056, 13057, 13058, 13059, 13060, 13061, 13062, 13063, 13064, 13065, 13066, 13067, 13068, 13069, 13070, 13071, 13072, 13073, 13074, 13075, 13076, 13077, 13078, 13079, 13080, 13081, 13082, 13083, 13084, 13085, 13086, 13087, 13088, 13089, 13090, 13091, 13092, 13093, 13094, 13095, 13096, 13097, 13098, 13099, 13100, 13101, 13102, 13103, 13104, 13105, 13106, 13107, 13108, 13109, 13110, 13111, 13112, 13113, 13114, 13115, 13116, 13117, 13118, 13119, 13120, 13121, 13122, 13123, 13124, 13125, 13126, 13127, 13128, 13129, 13130, 13131, 13132, 13133, 13134, 13135, 13136, 13137, 13138, 13139, 13140, 13141, 13142, 13143, 13144, 13145, 13146, 13147, 13148, 13149, 13150, 13151, 13152, 13153, 13154, 13155, 13156, 13157, 13158, 13159, 13160, 13161, 13162, 13163, 13164, 13165, 13166, 13167, 13168, 13169, 13170, 13171, 13172, 13173, 13174, 13175, 13176, 13177, 13178, 13179, 13180, 13181, 13182, 13183, 13184, 13185, 13186, 13187, 13188, 13189, 13190, 13191, 13192, 13193, 13194, 13195, 13196, 13197, 13198, 13199, 13200, 13201, 13202, 13203, 13204, 13205, 13206, 13207, 13208, 13209, 13210, 13211, 13212, 13213, 13214, 13215, 13216, 13217, 13218, 13219, 13220, 13221, 13222, 13223, 13224, 13225, 13226, 13227, 13228, 13229, 13230, 13231, 13232, 13233, 13234, 13235, 13236, 13237, 13238, 13239, 13240, 13241, 13242, 13243, 13244, 13245, 13246, 13247, 13248, 13249, 13250, 13251, 13252, 13253, 13254, 13255, 13256, 13257, 13258, 13259, 13260, 13261, 13262, 13263, 13264, 13265, 13266, 13267, 13268, 13269, 13270, 13271, 13272, 13273, 13274, 13275, 13276, 13277, 13278, 13279, 13280, 13281, 13282, 13283, 13284, 13285, 13286, 13287, 13288, 13289, 13290, 13291, 13292, 13293, 13294, 13295, 13296, 13297, 13298, 13299, 13300, 13301, 13302, 13303, 13304, 13305, 13306, 13307, 13308, 13309, 13310, 13311, 13312, 13313, 13314, 13315, 13316, 13317, 13318, 13319, 13320, 13321, 13322, 13323, 13324, 13325, 13326, 13327, 13328, 13329, 13330, 13331, 13332, 13333, 13334, 13335, 13336, 13337, 13338, 13339, 13340, 13341, 13342, 13343, 13344, 13345, 13346, 13347, 13348, 13349, 13350, 13351, 13352, 13353, 13354, 13355, 13356, 13357, 13358, 13359, 13360, 13361, 13362, 13363, 13364, 13365, 13366, 13367, 13368, 13369, 13370, 13371, 13372, 13373, 13374, 13375, 13376, 13377, 13378, 13379, 13380, 13381, 13382, 13383, 13384, 13385, 13386, 13387, 13388, 13389, 13390, 13391, 13392, 13393, 13394, 13395, 13396, 13397, 13398, 13399, 13400, 13401, 13402, 13403, 13404, 13405, 13406, 13407, 13408, 13409, 13410, 13411, 13412, 13413, 13414, 13415, 13416, 13417, 13418, 13419, 13420, 13421, 13422, 13423, 13424, 13425, 13426, 13427, 13428, 13429, 13430, 13431, 13432, 13433, 13434, 13435, 13436, 13437, 13438, 13439, 13440, 13441, 13442, 13443, 13444, 13445, 13446, 13447, 13448, 13449, 13450, 13451, 13452, 13453, 13454, 13455, 13456, 13457, 13458, 13459, 13460, 13461, 13462, 13463, 13464, 13465, 13466, 13467, 13468, 13469, 13470, 13471, 13472, 13473, 13474, 13475, 13476, 13477, 13478, 13479, 13480, 13481, 13482, 13483, 13484, 13485, 13486, 13487, 13488, 13489, 13490, 13491, 13492, 13493, 13494, 13495, 13496, 13497, 13498, 13499, 13500, 13501, 13502, 13503, 13504, 13505, 13506, 13507, 13508, 13509, 13510, 13511, 13512, 13513, 13514, 13515, 13516, 13517, 13518, 13519, 13520, 13521, 13522, 13523, 13524, 13525, 13526, 13527, 13528, 13529, 13530, 13531, 13532, 13533, 13534, 13535, 13536, 13537, 13538, 13539, 13540, 13541, 13542, 13543, 13544, 13545, 13546, 13547, 13548, 13549, 13550, 13551, 13552, 13553, 13554, 13555, 13556, 13557, 13558, 13559, 13560, 13561, 13562, 13563, 13564, 13565, 13566, 13567, 13568, 13569, 13570, 13571, 13572, 13573, 13574, 13575, 13576, 13577, 13578, 13579, 13580, 13581, 13582, 13583, 13584, 13585, 13586, 13587, 13588, 13589, 13590, 13591, 13592, 13593, 13594, 13595, 13596, 13597, 13598, 13599, 13600, 13601, 13602, 13603, 13604, 13605, 13606, 13607, 13608, 13609, 13610, 13611, 13612, 13613, 13614, 13615, 13616, 13617, 13618, 13619, 13620, 13621, 13622, 13623, 13624, 13625, 13626, 13627, 13628, 13629, 13630, 13631, 13632, 13633, 13634, 13635, 13636, 13637, 13638, 13639, 13640, 13641, 13642, 13643, 13644, 13645, 13646, 13647, 13648, 13649, 13650, 13651, 13652, 13653, 13654, 13655, 13656, 13657, 13658, 13659, 13660, 13661, 13662, 13663, 13664, 13665, 13666, 13667, 13668, 13669, 13670, 13671, 13672, 13673, 13674, 13675, 13676, 13677, 13678, 13679, 13680, 13681, 13682, 13683, 13684, 13685, 13686, 13687, 13688, 13689, 13690, 13691, 13692, 13693, 13694, 13695, 13696, 13697, 13698, 13699, 13700, 13701, 13702, 13703, 13704, 13705, 13706, 13707, 13708, 13709, 13710, 13711, 13712, 13713, 13714, 13715, 13716, 13717, 13718, 13719, 13720, 13721, 13722, 13723, 13724, 13725, 13726, 13727, 13728, 13729, 13730, 13731, 13732, 13733, 13734, 13735, 13736, 13737, 13738, 13739, 13740, 13741, 13742, 13743, 13744, 13745, 13746, 13747, 13748, 13749, 13750, 13751, 13752, 13753, 13754, 13755, 13756, 13757, 13758, 13759, 13760, 13761, 13762, 13763, 13764, 13765, 13766, 13767, 13768, 13769, 13770, 13771, 13772, 13773, 13774, 13775, 13776, 13777, 13778, 13779, 13780, 13781, 13782, 13783, 13784, 13785, 13786, 13787, 13788, 13789, 13790, 13791, 13792, 13793, 13794, 13795, 13796, 13797, 13798, 13799, 13800, 13801, 13802, 13803, 13804, 13805, 13806, 13807, 13808, 13809, 13810, 13811, 13812, 13813, 13814, 13815, 13816, 13817, 13818, 13819, 13820, 13821, 13822, 13823, 13824, 13825, 13826, 13827, 13828, 13829, 13830, 13831, 13832, 13833, 13834, 13835, 13836, 13837, 13838, 13839, 13840, 13841, 13842, 13843, 13844, 13845, 13846, 13847, 13848, 13849, 13850, 13851, 13852, 13853, 13854, 13855, 13856, 13857, 13858, 13859, 13860, 13861, 13862, 13863, 13864, 13865, 13866, 13867, 13868, 13869, 13870, 13871, 13872, 13873, 13874, 13875, 13876, 13877, 13878, 13879, 13880, 13881, 13882, 13883, 13884, 13885, 13886, 13887, 13888, 13889, 13890, 13891, 13892, 13893, 13894, 13895, 13896, 13897, 13898, 13899, 13900, 13901, 13902, 13903, 13904, 13905, 13906, 13907, 13908, 13909, 13910, 13911, 13912, 13913, 13914, 13915, 13916, 13917, 13918, 13919, 13920, 13921, 13922, 13923, 13924, 13925, 13926, 13927, 13928, 13929, 13930, 13931, 13932, 13933, 13934, 13935, 13936, 13937, 13938, 13939, 13940, 13941, 13942, 13943, 13944, 13945, 13946, 13947, 13948, 13949, 13950, 13951, 13952, 13953, 13954, 13955, 13956, 13957, 13958, 13959, 13960, 13961, 13962, 13963, 13964, 13965, 13966, 13967, 13968, 13969, 13970, 13971, 13972, 13973, 13974, 13975, 13976, 13977, 13978, 13979, 13980, 13981, 13982, 13983, 13984, 13985, 13986, 13987, 13988, 13989, 13990, 13991, 13992, 13993, 13994, 13995, 13996, 13997, 13998, 13999, 14000, 14001, 14002, 14003, 14004, 14005, 14006, 14007, 14008, 14009, 14010, 14011, 14012, 14013, 14014, 14015, 14016, 14017, 14018, 14019, 14020, 14021, 14022, 14023, 14024, 14025, 14026, 14027, 14028, 14029, 14030, 14031, 14032, 14033, 14034, 14035, 14036, 14037, 14038, 14039, 14040, 14041, 14042, 14043, 14044, 14045, 14046, 14047, 14048, 14049, 14050, 14051, 14052, 14053, 14054, 14055, 14056, 14057, 14058, 14059, 14060, 14061, 14062, 14063, 14064, 14065, 14066, 14067, 14068, 14069, 14070, 14071, 14072, 14073, 14074, 14075, 14076, 14077, 14078, 14079, 14080, 14081, 14082, 14083, 14084, 14085, 14086, 14087, 14088, 14089, 14090, 14091, 14092, 14093, 14094, 14095, 14096, 14097, 14098, 14099, 14100, 14101, 14102, 14103, 14104, 14105, 14106, 14107, 14108, 14109, 14110, 14111, 14112, 14113, 14114, 14115, 14116, 14117, 14118, 14119, 14120, 14121, 14122, 14123, 14124, 14125, 14126, 14127, 14128, 14129, 14130, 14131, 14132, 14133, 14134, 14135, 14136, 14137, 14138, 14139, 14140, 14141, 14142, 14143, 14144, 14145, 14146, 14147, 14148, 14149, 14150, 14151, 14152, 14153, 14154, 14155, 14156, 14157, 14158, 14159, 14160, 14161, 14162, 14163, 14164, 14165, 14166, 14167, 14168, 14169, 14170, 14171, 14172, 14173, 14174, 14175, 14176, 14177, 14178, 14179, 14180, 14181, 14182, 14183, 14184, 14185, 14186, 14187, 14188, 14189, 14190, 14191, 14192, 14193, 14194, 14195, 14196, 14197, 14198, 14199, 14200, 14201, 14202, 14203, 14204, 14205, 14206, 14207, 14208, 14209, 14210, 14211, 14212, 14213, 14214, 14215, 14216, 14217, 14218, 14219, 14220, 14221, 14222, 14223, 14224, 14225, 14226, 14227, 14228, 14229, 14230, 14231, 14232, 14233, 14234, 14235, 14236, 14237, 14238, 14239, 14240, 14241, 14242, 14243, 14244, 14245, 14246, 14247, 14248, 14249, 14250, 14251, 14252, 14253, 14254, 14255, 14256, 14257, 14258, 14259, 14260, 14261, 14262, 14263, 14264, 14265, 14266, 14267, 14268, 14269, 14270, 14271, 14272, 14273, 14274, 14275, 14276, 14277, 14278, 14279, 14280, 14281, 14282, 14283, 14284, 14285, 14286, 14287, 14288, 14289, 14290, 14291, 14292, 14293, 14294, 14295, 14296, 14297, 14298, 14299, 14300, 14301, 14302, 14303, 14304, 14305, 14306, 14307, 14308, 14309, 14310, 14311, 14312, 14313, 14314, 14315, 14316, 14317, 14318, 14319, 14320, 14321, 14322, 14323, 14324, 14325, 14326, 14327, 14328, 14329, 14330, 14331, 14332, 14333, 14334, 14335, 14336, 14337, 14338, 14339, 14340, 14341, 14342, 14343, 14344, 14345, 14346, 14347, 14348, 14349, 14350, 14351, 14352, 14353, 14354, 14355, 14356, 14357, 14358, 14359, 14360, 14361, 14362, 14363, 14364, 14365, 14366, 14367, 14368, 14369, 14370, 14371, 14372, 14373, 14374, 14375, 14376, 14377, 14378, 14379, 14380, 14381, 14382, 14383, 14384, 14385, 14386, 14387, 14388, 14389, 14390, 14391, 14392, 14393, 14394, 14395, 14396, 14397, 14398, 14399, 14400, 14401, 14402, 14403, 14404, 14405, 14406, 14407, 14408, 14409, 14410, 14411, 14412, 14413, 14414, 14415, 14416, 14417, 14418, 14419, 14420, 14421, 14422, 14423, 14424, 14425, 14426, 14427, 14428, 14429, 14430, 14431, 14432, 14433, 14434, 14435, 14436, 14437, 14438, 14439, 14440, 14441, 14442, 14443, 14444, 14445, 14446, 14447, 14448, 14449, 14450, 14451, 14452, 14453, 14454, 14455, 14456, 14457, 14458, 14459, 14460, 14461, 14462, 14463, 14464, 14465, 14466, 14467, 14468, 14469, 14470, 14471, 14472, 14473, 14474, 14475, 14476, 14477, 14478, 14479, 14480, 14481, 14482, 14483, 14484, 14485, 14486, 14487, 14488, 14489, 14490, 14491, 14492, 14493, 14494, 14495, 14496, 14497, 14498, 14499, 14500, 14501, 14502, 14503, 14504, 14505, 14506, 14507, 14508, 14509, 14510, 14511, 14512, 14513, 14514, 14515, 14516, 14517, 14518, 14519, 14520, 14521, 14522, 14523, 14524, 14525, 14526, 14527, 14528, 14529, 14530, 14531, 14532, 14533, 14534, 14535, 14536, 14537, 14538, 14539, 14540, 14541, 14542, 14543, 14544, 14545, 14546, 14547, 14548, 14549, 14550, 14551, 14552, 14553, 14554, 14555, 14556, 14557, 14558, 14559, 14560, 14561, 14562, 14563, 14564, 14565, 14566, 14567, 14568, 14569, 14570, 14571, 14572, 14573, 14574, 14575, 14576, 14577, 14578, 14579, 14580, 14581, 14582, 14583, 14584, 14585, 14586, 14587, 14588, 14589, 14590, 14591, 14592, 14593, 14594, 14595, 14596, 14597, 14598, 14599, 14600, 14601, 14602, 14603, 14604, 14605, 14606, 14607, 14608, 14609, 14610, 14611, 14612, 14613, 14614, 14615, 14616, 14617, 14618, 14619, 14620, 14621, 14622, 14623, 14624, 14625, 14626, 14627, 14628, 14629, 14630, 14631, 14632, 14633, 14634, 14635, 14636, 14637, 14638, 14639, 14640, 14641, 14642, 14643, 14644, 14645, 14646, 14647, 14648, 14649, 14650, 14651, 14652, 14653, 14654, 14655, 14656, 14657, 14658, 14659, 14660, 14661, 14662, 14663, 14664, 14665, 14666, 14667, 14668, 14669, 14670, 14671, 14672, 14673, 14674, 14675, 14676, 14677, 14678, 14679, 14680, 14681, 14682, 14683, 14684, 14685, 14686, 14687, 14688, 14689, 14690, 14691, 14692, 14693, 14694, 14695, 14696, 14697, 14698, 14699, 14700, 14701, 14702, 14703, 14704, 14705, 14706, 14707, 14708, 14709, 14710, 14711, 14712, 14713, 14714, 14715, 14716, 14717, 14718, 14719, 14720, 14721, 14722, 14723, 14724, 14725, 14726, 14727, 14728, 14729, 14730, 14731, 14732, 14733, 14734, 14735, 14736, 14737, 14738, 14739, 14740, 14741, 14742, 14743, 14744, 14745, 14746, 14747, 14748, 14749, 14750, 14751, 14752, 14753, 14754, 14755, 14756, 14757, 14758, 14759, 14760, 14761, 14762, 14763, 14764, 14765, 14766, 14767, 14768, 14769, 14770, 14771, 14772, 14773, 14774, 14775, 14776, 14777, 14778, 14779, 14780, 14781, 14782, 14783, 14784, 14785, 14786, 14787, 14788, 14789, 14790, 14791, 14792, 14793, 14794, 14795, 14796, 14797, 14798, 14799, 14800, 14801, 14802, 14803, 14804, 14805, 14806, 14807, 14808, 14809, 14810, 14811, 14812, 14813, 14814, 14815, 14816, 14817, 14818, 14819, 14820, 14821, 14822, 14823, 14824, 14825, 14826, 14827, 14828, 14829, 14830, 14831, 14832, 14833, 14834, 14835, 14836, 14837, 14838, 14839, 14840, 14841, 14842, 14843, 14844, 14845, 14846, 14847, 14848, 14849, 14850, 14851, 14852, 14853, 14854, 14855, 14856, 14857, 14858, 14859, 14860, 14861, 14862, 14863, 14864, 14865, 14866, 14867, 14868, 14869, 14870, 14871, 14872, 14873, 14874, 14875, 14876, 14877, 14878, 14879, 14880, 14881, 14882, 14883, 14884, 14885, 14886, 14887, 14888, 14889, 14890, 14891, 14892, 14893, 14894, 14895, 14896, 14897, 14898, 14899, 14900, 14901, 14902, 14903, 14904, 14905, 14906, 14907, 14908, 14909, 14910, 14911, 14912, 14913, 14914, 14915, 14916, 14917, 14918, 14919, 14920, 14921, 14922, 14923, 14924, 14925, 14926, 14927, 14928, 14929, 14930, 14931, 14932, 14933, 14934, 14935, 14936, 14937, 14938, 14939, 14940, 14941, 14942, 14943, 14944, 14945, 14946, 14947, 14948, 14949, 14950, 14951, 14952, 14953, 14954, 14955, 14956, 14957, 14958, 14959, 14960, 14961, 14962, 14963, 14964, 14965, 14966, 14967, 14968, 14969, 14970, 14971, 14972, 14973, 14974, 14975, 14976, 14977, 14978, 14979, 14980, 14981, 14982, 14983, 14984, 14985, 14986, 14987, 14988, 14989, 14990, 14991, 14992, 14993, 14994, 14995, 14996, 14997, 14998, 14999, 15000, 15001, 15002, 15003, 15004, 15005, 15006, 15007, 15008, 15009, 15010, 15011, 15012, 15013, 15014, 15015, 15016, 15017, 15018, 15019, 15020, 15021, 15022, 15023, 15024, 15025, 15026, 15027, 15028, 15029, 15030, 15031, 15032, 15033, 15034, 15035, 15036, 15037, 15038, 15039, 15040, 15041, 15042, 15043, 15044, 15045, 15046, 15047, 15048, 15049, 15050, 15051, 15052, 15053, 15054, 15055, 15056, 15057, 15058, 15059, 15060, 15061, 15062, 15063, 15064, 15065, 15066, 15067, 15068, 15069, 15070, 15071, 15072, 15073, 15074, 15075, 15076, 15077, 15078, 15079, 15080, 15081, 15082, 15083, 15084, 15085, 15086, 15087, 15088, 15089, 15090, 15091, 15092, 15093, 15094, 15095, 15096, 15097, 15098, 15099, 15100, 15101, 15102, 15103, 15104, 15105, 15106, 15107, 15108, 15109, 15110, 15111, 15112, 15113, 15114, 15115, 15116, 15117, 15118, 15119, 15120, 15121, 15122, 15123, 15124, 15125, 15126, 15127, 15128, 15129, 15130, 15131, 15132, 15133, 15134, 15135, 15136, 15137, 15138, 15139, 15140, 15141, 15142, 15143, 15144, 15145, 15146, 15147, 15148, 15149, 15150, 15151, 15152, 15153, 15154, 15155, 15156, 15157, 15158, 15159, 15160, 15161, 15162, 15163, 15164, 15165, 15166, 15167, 15168, 15169, 15170, 15171, 15172, 15173, 15174, 15175, 15176, 15177, 15178, 15179, 15180, 15181, 15182, 15183, 15184, 15185, 15186, 15187, 15188, 15189, 15190, 15191, 15192, 15193, 15194, 15195, 15196, 15197, 15198, 15199, 15200, 15201, 15202, 15203, 15204, 15205, 15206, 15207, 15208, 15209, 15210, 15211, 15212, 15213, 15214, 15215, 15216, 15217, 15218, 15219, 15220, 15221, 15222, 15223, 15224, 15225, 15226, 15227, 15228, 15229, 15230, 15231, 15232, 15233, 15234, 15235, 15236, 15237, 15238, 15239, 15240, 15241, 15242, 15243, 15244, 15245, 15246, 15247, 15248, 15249, 15250, 15251, 15252, 15253, 15254, 15255, 15256, 15257, 15258, 15259, 15260, 15261, 15262, 15263, 15264, 15265, 15266, 15267, 15268, 15269, 15270, 15271, 15272, 15273, 15274, 15275, 15276, 15277, 15278, 15279, 15280, 15281, 15282, 15283, 15284, 15285, 15286, 15287, 15288, 15289, 15290, 15291, 15292, 15293, 15294, 15295, 15296, 15297, 15298, 15299, 15300, 15301, 15302, 15303, 15304, 15305, 15306, 15307, 15308, 15309, 15310, 15311, 15312, 15313, 15314, 15315, 15316, 15317, 15318, 15319, 15320, 15321, 15322, 15323, 15324, 15325, 15326, 15327, 15328, 15329, 15330, 15331, 15332, 15333, 15334, 15335, 15336, 15337, 15338, 15339, 15340, 15341, 15342, 15343, 15344, 15345, 15346, 15347, 15348, 15349, 15350, 15351, 15352, 15353, 15354, 15355, 15356, 15357, 15358, 15359, 15360, 15361, 15362, 15363, 15364, 15365, 15366, 15367, 15368, 15369, 15370, 15371, 15372, 15373, 15374, 15375, 15376, 15377, 15378, 15379, 15380, 15381, 15382, 15383, 15384, 15385, 15386, 15387, 15388, 15389, 15390, 15391, 15392, 15393, 15394, 15395, 15396, 15397, 15398, 15399, 15400, 15401, 15402, 15403, 15404, 15405, 15406, 15407, 15408, 15409, 15410, 15411, 15412, 15413, 15414, 15415, 15416, 15417, 15418, 15419, 15420, 15421, 15422, 15423, 15424, 15425, 15426, 15427, 15428, 15429, 15430, 15431, 15432, 15433, 15434, 15435, 15436, 15437, 15438, 15439, 15440, 15441, 15442, 15443, 15444, 15445, 15446, 15447, 15448, 15449, 15450, 15451, 15452, 15453, 15454, 15455, 15456, 15457, 15458, 15459, 15460, 15461, 15462, 15463, 15464, 15465, 15466, 15467, 15468, 15469, 15470, 15471, 15472, 15473, 15474, 15475, 15476, 15477, 15478, 15479, 15480, 15481, 15482, 15483, 15484, 15485, 15486, 15487, 15488, 15489, 15490, 15491, 15492, 15493, 15494, 15495, 15496, 15497, 15498, 15499, 15500, 15501, 15502, 15503, 15504, 15505, 15506, 15507, 15508, 15509, 15510, 15511, 15512, 15513, 15514, 15515, 15516, 15517, 15518, 15519, 15520, 15521, 15522, 15523, 15524, 15525, 15526, 15527, 15528, 15529, 15530, 15531, 15532, 15533, 15534, 15535, 15536, 15537, 15538, 15539, 15540, 15541, 15542, 15543, 15544, 15545, 15546, 15547, 15548, 15549, 15550, 15551, 15552, 15553, 15554, 15555, 15556, 15557, 15558, 15559, 15560, 15561, 15562, 15563, 15564, 15565, 15566, 15567, 15568, 15569, 15570, 15571, 15572, 15573, 15574, 15575, 15576, 15577, 15578, 15579, 15580, 15581, 15582, 15583, 15584, 15585, 15586, 15587, 15588, 15589, 15590, 15591, 15592, 15593, 15594, 15595, 15596, 15597, 15598, 15599, 15600, 15601, 15602, 15603, 15604, 15605, 15606, 15607, 15608, 15609, 15610, 15611, 15612, 15613, 15614, 15615, 15616, 15617, 15618, 15619, 15620, 15621, 15622, 15623, 15624, 15625, 15626, 15627, 15628, 15629, 15630, 15631, 15632, 15633, 15634, 15635, 15636, 15637, 15638, 15639, 15640, 15641, 15642, 15643, 15644, 15645, 15646, 15647, 15648, 15649, 15650, 15651, 15652, 15653, 15654, 15655, 15656, 15657, 15658, 15659, 15660, 15661, 15662, 15663, 15664, 15665, 15666, 15667, 15668, 15669, 15670, 15671, 15672, 15673, 15674, 15675, 15676, 15677, 15678, 15679, 15680, 15681, 15682, 15683, 15684, 15685, 15686, 15687, 15688, 15689, 15690, 15691, 15692, 15693, 15694, 15695, 15696, 15697, 15698, 15699, 15700, 15701, 15702, 15703, 15704, 15705, 15706, 15707, 15708, 15709, 15710, 15711, 15712, 15713, 15714, 15715, 15716, 15717, 15718, 15719, 15720, 15721, 15722, 15723, 15724, 15725, 15726, 15727, 15728, 15729, 15730, 15731, 15732, 15733, 15734, 15735, 15736, 15737, 15738, 15739, 15740, 15741, 15742, 15743, 15744, 15745, 15746, 15747, 15748, 15749, 15750, 15751, 15752, 15753, 15754, 15755, 15756, 15757, 15758, 15759, 15760, 15761, 15762, 15763, 15764, 15765, 15766, 15767, 15768, 15769, 15770, 15771, 15772, 15773, 15774, 15775, 15776, 15777, 15778, 15779, 15780, 15781, 15782, 15783, 15784, 15785, 15786, 15787, 15788, 15789, 15790, 15791, 15792, 15793, 15794, 15795, 15796, 15797, 15798, 15799, 15800, 15801, 15802, 15803, 15804, 15805, 15806, 15807, 15808, 15809, 15810, 15811, 15812, 15813, 15814, 15815, 15816, 15817, 15818, 15819, 15820, 15821, 15822, 15823, 15824, 15825, 15826, 15827, 15828, 15829, 15830, 15831, 15832, 15833, 15834, 15835, 15836, 15837, 15838, 15839, 15840, 15841, 15842, 15843, 15844, 15845, 15846, 15847, 15848, 15849, 15850, 15851, 15852, 15853, 15854, 15855, 15856, 15857, 15858, 15859, 15860, 15861, 15862, 15863, 15864, 15865, 15866, 15867, 15868, 15869, 15870, 15871, 15872, 15873, 15874, 15875, 15876, 15877, 15878, 15879, 15880, 15881, 15882, 15883, 15884, 15885, 15886, 15887, 15888, 15889, 15890, 15891, 15892, 15893, 15894, 15895, 15896, 15897, 15898, 15899, 15900, 15901, 15902, 15903, 15904, 15905, 15906, 15907, 15908, 15909, 15910, 15911, 15912, 15913, 15914, 15915, 15916, 15917, 15918, 15919, 15920, 15921, 15922, 15923, 15924, 15925, 15926, 15927, 15928, 15929, 15930, 15931, 15932, 15933, 15934, 15935, 15936, 15937, 15938, 15939, 15940, 15941, 15942, 15943, 15944, 15945, 15946, 15947, 15948, 15949, 15950, 15951, 15952, 15953, 15954, 15955, 15956, 15957, 15958, 15959, 15960, 15961, 15962, 15963, 15964, 15965, 15966, 15967, 15968, 15969, 15970, 15971, 15972, 15973, 15974, 15975, 15976, 15977, 15978, 15979, 15980, 15981, 15982, 15983, 15984, 15985, 15986, 15987, 15988, 15989, 15990, 15991, 15992, 15993, 15994, 15995, 15996, 15997, 15998, 15999, 16000, 16001, 16002, 16003, 16004, 16005, 16006, 16007, 16008, 16009, 16010, 16011, 16012, 16013, 16014, 16015, 16016, 16017, 16018, 16019, 16020, 16021, 16022, 16023, 16024, 16025, 16026, 16027, 16028, 16029, 16030, 16031, 16032, 16033, 16034, 16035, 16036, 16037, 16038, 16039, 16040, 16041, 16042, 16043, 16044, 16045, 16046, 16047, 16048, 16049, 16050, 16051, 16052, 16053, 16054, 16055, 16056, 16057, 16058, 16059, 16060, 16061, 16062, 16063, 16064, 16065, 16066, 16067, 16068, 16069, 16070, 16071, 16072, 16073, 16074, 16075, 16076, 16077, 16078, 16079, 16080, 16081, 16082, 16083, 16084, 16085, 16086, 16087, 16088, 16089, 16090, 16091, 16092, 16093, 16094, 16095, 16096, 16097, 16098, 16099, 16100, 16101, 16102, 16103, 16104, 16105, 16106, 16107, 16108, 16109, 16110, 16111, 16112, 16113, 16114, 16115, 16116, 16117, 16118, 16119, 16120, 16121, 16122, 16123, 16124, 16125, 16126, 16127, 16128, 16129, 16130, 16131, 16132, 16133, 16134, 16135, 16136, 16137, 16138, 16139, 16140, 16141, 16142, 16143, 16144, 16145, 16146, 16147, 16148, 16149, 16150, 16151, 16152, 16153, 16154, 16155, 16156, 16157, 16158, 16159, 16160, 16161, 16162, 16163, 16164, 16165, 16166, 16167, 16168, 16169, 16170, 16171, 16172, 16173, 16174, 16175, 16176, 16177, 16178, 16179, 16180, 16181, 16182, 16183, 16184, 16185, 16186, 16187, 16188, 16189, 16190, 16191, 16192, 16193, 16194, 16195, 16196, 16197, 16198, 16199, 16200, 16201, 16202, 16203, 16204, 16205, 16206, 16207, 16208, 16209, 16210, 16211, 16212, 16213, 16214, 16215, 16216, 16217, 16218, 16219, 16220, 16221, 16222, 16223, 16224, 16225, 16226, 16227, 16228, 16229, 16230, 16231, 16232, 16233, 16234, 16235, 16236, 16237, 16238, 16239, 16240, 16241, 16242, 16243, 16244, 16245, 16246, 16247, 16248, 16249, 16250, 16251, 16252, 16253, 16254, 16255, 16256, 16257, 16258, 16259, 16260, 16261, 16262, 16263, 16264, 16265, 16266, 16267, 16268, 16269, 16270, 16271, 16272, 16273, 16274, 16275, 16276, 16277, 16278, 16279, 16280, 16281, 16282, 16283, 16284, 16285, 16286, 16287, 16288, 16289, 16290, 16291, 16292, 16293, 16294, 16295, 16296, 16297, 16298, 16299, 16300, 16301, 16302, 16303, 16304, 16305, 16306, 16307, 16308, 16309, 16310, 16311, 16312, 16313, 16314, 16315, 16316, 16317, 16318, 16319, 16320, 16321, 16322, 16323, 16324, 16325, 16326, 16327, 16328, 16329, 16330, 16331, 16332, 16333, 16334, 16335, 16336, 16337, 16338, 16339, 16340, 16341, 16342, 16343, 16344, 16345, 16346, 16347, 16348, 16349, 16350, 16351, 16352, 16353, 16354, 16355, 16356, 16357, 16358, 16359, 16360, 16361, 16362, 16363, 16364, 16365, 16366, 16367, 16368, 16369, 16370, 16371, 16372, 16373, 16374, 16375, 16376, 16377, 16378, 16379, 16380, 16381, 16382, 16383, 16384, 16385, 16386, 16387, 16388, 16389, 16390, 16391, 16392, 16393, 16394, 16395, 16396, 16397, 16398, 16399, 16400, 16401, 16402, 16403, 16404, 16405, 16406, 16407, 16408, 16409, 16410, 16411, 16412, 16413, 16414, 16415, 16416, 16417, 16418, 16419, 16420, 16421, 16422, 16423, 16424, 16425, 16426, 16427, 16428, 16429, 16430, 16431, 16432, 16433, 16434, 16435, 16436, 16437, 16438, 16439, 16440, 16441, 16442, 16443, 16444, 16445, 16446, 16447, 16448, 16449, 16450, 16451, 16452, 16453, 16454, 16455, 16456, 16457, 16458, 16459, 16460, 16461, 16462, 16463, 16464, 16465, 16466, 16467, 16468, 16469, 16470, 16471, 16472, 16473, 16474, 16475, 16476, 16477, 16478, 16479, 16480, 16481, 16482, 16483, 16484, 16485, 16486, 16487, 16488, 16489, 16490, 16491, 16492, 16493, 16494, 16495, 16496, 16497, 16498, 16499, 16500, 16501, 16502, 16503, 16504, 16505, 16506, 16507, 16508, 16509, 16510, 16511, 16512, 16513, 16514, 16515, 16516, 16517, 16518, 16519, 16520, 16521, 16522, 16523, 16524, 16525, 16526, 16527, 16528, 16529, 16530, 16531, 16532, 16533, 16534, 16535, 16536, 16537, 16538, 16539, 16540, 16541, 16542, 16543, 16544, 16545, 16546, 16547, 16548, 16549, 16550, 16551, 16552, 16553, 16554, 16555, 16556, 16557, 16558, 16559, 16560, 16561, 16562, 16563, 16564, 16565, 16566, 16567, 16568, 16569, 16570, 16571, 16572, 16573, 16574, 16575, 16576, 16577, 16578, 16579, 16580, 16581, 16582, 16583, 16584, 16585, 16586, 16587, 16588, 16589, 16590, 16591, 16592, 16593, 16594, 16595, 16596, 16597, 16598, 16599, 16600, 16601, 16602, 16603, 16604, 16605, 16606, 16607, 16608, 16609, 16610, 16611, 16612, 16613, 16614, 16615, 16616, 16617, 16618, 16619, 16620, 16621, 16622, 16623, 16624, 16625, 16626, 16627, 16628, 16629, 16630, 16631, 16632, 16633, 16634, 16635, 16636, 16637, 16638, 16639, 16640, 16641, 16642, 16643, 16644, 16645, 16646, 16647, 16648, 16649, 16650, 16651, 16652, 16653, 16654, 16655, 16656, 16657, 16658, 16659, 16660, 16661, 16662, 16663, 16664, 16665, 16666, 16667, 16668, 16669, 16670, 16671, 16672, 16673, 16674, 16675, 16676, 16677, 16678, 16679, 16680, 16681, 16682, 16683, 16684, 16685, 16686, 16687, 16688, 16689, 16690, 16691, 16692, 16693, 16694, 16695, 16696, 16697, 16698, 16699, 16700, 16701, 16702, 16703, 16704, 16705, 16706, 16707, 16708, 16709, 16710, 16711, 16712, 16713, 16714, 16715, 16716, 16717, 16718, 16719, 16720, 16721, 16722, 16723, 16724, 16725, 16726, 16727, 16728, 16729, 16730, 16731, 16732, 16733, 16734, 16735, 16736, 16737, 16738, 16739, 16740, 16741, 16742, 16743, 16744, 16745, 16746, 16747, 16748, 16749, 16750, 16751, 16752, 16753, 16754, 16755, 16756, 16757, 16758, 16759, 16760, 16761, 16762, 16763, 16764, 16765, 16766, 16767, 16768, 16769, 16770, 16771, 16772, 16773, 16774, 16775, 16776, 16777, 16778, 16779, 16780, 16781, 16782, 16783, 16784, 16785, 16786, 16787, 16788, 16789, 16790, 16791, 16792, 16793, 16794, 16795, 16796, 16797, 16798, 16799, 16800, 16801, 16802, 16803, 16804, 16805, 16806, 16807, 16808, 16809, 16810, 16811, 16812, 16813, 16814, 16815, 16816, 16817, 16818, 16819, 16820, 16821, 16822, 16823, 16824, 16825, 16826, 16827, 16828, 16829, 16830, 16831, 16832, 16833, 16834, 16835, 16836, 16837, 16838, 16839, 16840, 16841, 16842, 16843, 16844, 16845, 16846, 16847, 16848, 16849, 16850, 16851, 16852, 16853, 16854, 16855, 16856, 16857, 16858, 16859, 16860, 16861, 16862, 16863, 16864, 16865, 16866, 16867, 16868, 16869, 16870, 16871, 16872, 16873, 16874, 16875, 16876, 16877, 16878, 16879, 16880, 16881, 16882, 16883, 16884, 16885, 16886, 16887, 16888, 16889, 16890, 16891, 16892, 16893, 16894, 16895, 16896, 16897, 16898, 16899, 16900, 16901, 16902, 16903, 16904, 16905, 16906, 16907, 16908, 16909, 16910, 16911, 16912, 16913, 16914, 16915, 16916, 16917, 16918, 16919, 16920, 16921, 16922, 16923, 16924, 16925, 16926, 16927, 16928, 16929, 16930, 16931, 16932, 16933, 16934, 16935, 16936, 16937, 16938, 16939, 16940, 16941, 16942, 16943, 16944, 16945, 16946, 16947, 16948, 16949, 16950, 16951, 16952, 16953, 16954, 16955, 16956, 16957, 16958, 16959, 16960, 16961, 16962, 16963, 16964, 16965, 16966, 16967, 16968, 16969, 16970, 16971, 16972, 16973, 16974, 16975, 16976, 16977, 16978, 16979, 16980, 16981, 16982, 16983, 16984, 16985, 16986, 16987, 16988, 16989, 16990, 16991, 16992, 16993, 16994, 16995, 16996, 16997, 16998, 16999, 17000, 17001, 17002, 17003, 17004, 17005, 17006, 17007, 17008, 17009, 17010, 17011, 17012, 17013, 17014, 17015, 17016, 17017, 17018, 17019, 17020, 17021, 17022, 17023, 17024, 17025, 17026, 17027, 17028, 17029, 17030, 17031, 17032, 17033, 17034, 17035, 17036, 17037, 17038, 17039, 17040, 17041, 17042, 17043, 17044, 17045, 17046, 17047, 17048, 17049, 17050, 17051, 17052, 17053, 17054, 17055, 17056, 17057, 17058, 17059, 17060, 17061, 17062, 17063, 17064, 17065, 17066, 17067, 17068, 17069, 17070, 17071, 17072, 17073, 17074, 17075, 17076, 17077, 17078, 17079, 17080, 17081, 17082, 17083, 17084, 17085, 17086, 17087, 17088, 17089, 17090, 17091, 17092, 17093, 17094, 17095, 17096, 17097, 17098, 17099, 17100, 17101, 17102, 17103, 17104, 17105, 17106, 17107, 17108, 17109, 17110, 17111, 17112, 17113, 17114, 17115, 17116, 17117, 17118, 17119, 17120, 17121, 17122, 17123, 17124, 17125, 17126, 17127, 17128, 17129, 17130, 17131, 17132, 17133, 17134, 17135, 17136, 17137, 17138, 17139, 17140, 17141, 17142, 17143, 17144, 17145, 17146, 17147, 17148, 17149, 17150, 17151, 17152, 17153, 17154, 17155, 17156, 17157, 17158, 17159, 17160, 17161, 17162, 17163, 17164, 17165, 17166, 17167, 17168, 17169, 17170, 17171, 17172, 17173, 17174, 17175, 17176, 17177, 17178, 17179, 17180, 17181, 17182, 17183, 17184, 17185, 17186, 17187, 17188, 17189, 17190, 17191, 17192, 17193, 17194, 17195, 17196, 17197, 17198, 17199, 17200, 17201, 17202, 17203, 17204, 17205, 17206, 17207, 17208, 17209, 17210, 17211, 17212, 17213, 17214, 17215, 17216, 17217, 17218, 17219, 17220, 17221, 17222, 17223, 17224, 17225, 17226, 17227, 17228, 17229, 17230, 17231, 17232, 17233, 17234, 17235, 17236, 17237, 17238, 17239, 17240, 17241, 17242, 17243, 17244, 17245, 17246, 17247, 17248, 17249, 17250, 17251, 17252, 17253, 17254, 17255, 17256, 17257, 17258, 17259, 17260, 17261, 17262, 17263, 17264, 17265, 17266, 17267, 17268, 17269, 17270, 17271, 17272, 17273, 17274, 17275, 17276, 17277, 17278, 17279, 17280, 17281, 17282, 17283, 17284, 17285, 17286, 17287, 17288, 17289, 17290, 17291, 17292, 17293, 17294, 17295, 17296, 17297, 17298, 17299, 17300, 17301, 17302, 17303, 17304, 17305, 17306, 17307, 17308, 17309, 17310, 17311, 17312, 17313, 17314, 17315, 17316, 17317, 17318, 17319, 17320, 17321, 17322, 17323, 17324, 17325, 17326, 17327, 17328, 17329, 17330, 17331, 17332, 17333, 17334, 17335, 17336, 17337, 17338, 17339, 17340, 17341, 17342, 17343, 17344, 17345, 17346, 17347, 17348, 17349, 17350, 17351, 17352, 17353, 17354, 17355, 17356, 17357, 17358, 17359, 17360, 17361, 17362, 17363, 17364, 17365, 17366, 17367, 17368, 17369, 17370, 17371, 17372, 17373, 17374, 17375, 17376, 17377, 17378, 17379, 17380, 17381, 17382, 17383, 17384, 17385, 17386, 17387, 17388, 17389, 17390, 17391, 17392, 17393, 17394, 17395, 17396, 17397, 17398, 17399, 17400, 17401, 17402, 17403, 17404, 17405, 17406, 17407, 17408, 17409, 17410, 17411, 17412, 17413, 17414, 17415, 17416, 17417, 17418, 17419, 17420, 17421, 17422, 17423, 17424, 17425, 17426, 17427, 17428, 17429, 17430, 17431, 17432, 17433, 17434, 17435, 17436, 17437, 17438, 17439, 17440, 17441, 17442, 17443, 17444, 17445, 17446, 17447, 17448, 17449, 17450, 17451, 17452, 17453, 17454, 17455, 17456, 17457, 17458, 17459, 17460, 17461, 17462, 17463, 17464, 17465, 17466, 17467, 17468, 17469, 17470, 17471, 17472, 17473, 17474, 17475, 17476, 17477, 17478, 17479, 17480, 17481, 17482, 17483, 17484, 17485, 17486, 17487, 17488, 17489, 17490, 17491, 17492, 17493, 17494, 17495, 17496, 17497, 17498, 17499, 17500, 17501, 17502, 17503, 17504, 17505, 17506, 17507, 17508, 17509, 17510, 17511, 17512, 17513, 17514, 17515, 17516, 17517, 17518, 17519, 17520, 17521, 17522, 17523, 17524, 17525, 17526, 17527, 17528, 17529, 17530, 17531, 17532, 17533, 17534, 17535, 17536, 17537, 17538, 17539, 17540, 17541, 17542, 17543, 17544, 17545, 17546, 17547, 17548, 17549, 17550, 17551, 17552, 17553, 17554, 17555, 17556, 17557, 17558, 17559, 17560, 17561, 17562, 17563, 17564, 17565, 17566, 17567, 17568, 17569, 17570, 17571, 17572, 17573, 17574, 17575, 17576, 17577, 17578, 17579, 17580, 17581, 17582, 17583, 17584, 17585, 17586, 17587, 17588, 17589, 17590, 17591, 17592, 17593, 17594, 17595, 17596, 17597, 17598, 17599, 17600, 17601, 17602, 17603, 17604, 17605, 17606, 17607, 17608, 17609, 17610, 17611, 17612, 17613, 17614, 17615, 17616, 17617, 17618, 17619, 17620, 17621, 17622, 17623, 17624, 17625, 17626, 17627, 17628, 17629, 17630, 17631, 17632, 17633, 17634, 17635, 17636, 17637, 17638, 17639, 17640, 17641, 17642, 17643, 17644, 17645, 17646, 17647, 17648, 17649, 17650, 17651, 17652, 17653, 17654, 17655, 17656, 17657, 17658, 17659, 17660, 17661, 17662, 17663, 17664, 17665, 17666, 17667, 17668, 17669, 17670, 17671, 17672, 17673, 17674, 17675, 17676, 17677, 17678, 17679, 17680, 17681, 17682, 17683, 17684, 17685, 17686, 17687, 17688, 17689, 17690, 17691, 17692, 17693, 17694, 17695, 17696, 17697, 17698, 17699, 17700, 17701, 17702, 17703, 17704, 17705, 17706, 17707, 17708, 17709, 17710, 17711, 17712, 17713, 17714, 17715, 17716, 17717, 17718, 17719, 17720, 17721, 17722, 17723, 17724, 17725, 17726, 17727, 17728, 17729, 17730, 17731, 17732, 17733, 17734, 17735, 17736, 17737, 17738, 17739, 17740, 17741, 17742, 17743, 17744, 17745, 17746, 17747, 17748, 17749, 17750, 17751, 17752, 17753, 17754, 17755, 17756, 17757, 17758, 17759, 17760, 17761, 17762, 17763, 17764, 17765, 17766, 17767, 17768, 17769, 17770, 17771, 17772, 17773, 17774, 17775, 17776, 17777, 17778, 17779, 17780, 17781, 17782, 17783, 17784, 17785, 17786, 17787, 17788, 17789, 17790, 17791, 17792, 17793, 17794, 17795, 17796, 17797, 17798, 17799, 17800, 17801, 17802, 17803, 17804, 17805, 17806, 17807, 17808, 17809, 17810, 17811, 17812, 17813, 17814, 17815, 17816, 17817, 17818, 17819, 17820, 17821, 17822, 17823, 17824, 17825, 17826, 17827, 17828, 17829, 17830, 17831, 17832, 17833, 17834, 17835, 17836, 17837, 17838, 17839, 17840, 17841, 17842, 17843, 17844, 17845, 17846, 17847, 17848, 17849, 17850, 17851, 17852, 17853, 17854, 17855, 17856, 17857, 17858, 17859, 17860, 17861, 17862, 17863, 17864, 17865, 17866, 17867, 17868, 17869, 17870, 17871, 17872, 17873, 17874, 17875, 17876, 17877, 17878, 17879, 17880, 17881, 17882, 17883, 17884, 17885, 17886, 17887, 17888, 17889, 17890, 17891, 17892, 17893, 17894, 17895, 17896, 17897, 17898, 17899, 17900, 17901, 17902, 17903, 17904, 17905, 17906, 17907, 17908, 17909, 17910, 17911, 17912, 17913, 17914, 17915, 17916, 17917, 17918, 17919, 17920, 17921, 17922, 17923, 17924, 17925, 17926, 17927, 17928, 17929, 17930, 17931, 17932, 17933, 17934, 17935, 17936, 17937, 17938, 17939, 17940, 17941, 17942, 17943, 17944, 17945, 17946, 17947, 17948, 17949, 17950, 17951, 17952, 17953, 17954, 17955, 17956, 17957, 17958, 17959, 17960, 17961, 17962, 17963, 17964, 17965, 17966, 17967, 17968, 17969, 17970, 17971, 17972, 17973, 17974, 17975, 17976, 17977, 17978, 17979, 17980, 17981, 17982, 17983, 17984, 17985, 17986, 17987, 17988, 17989, 17990, 17991, 17992, 17993, 17994, 17995, 17996, 17997, 17998, 17999, 18000, 18001, 18002, 18003, 18004, 18005, 18006, 18007, 18008, 18009, 18010, 18011, 18012, 18013, 18014, 18015, 18016, 18017, 18018, 18019, 18020, 18021, 18022, 18023, 18024, 18025, 18026, 18027, 18028, 18029, 18030, 18031, 18032, 18033, 18034, 18035, 18036, 18037, 18038, 18039, 18040, 18041, 18042, 18043, 18044, 18045, 18046, 18047, 18048, 18049, 18050, 18051, 18052, 18053, 18054, 18055, 18056, 18057, 18058, 18059, 18060, 18061, 18062, 18063, 18064, 18065, 18066, 18067, 18068, 18069, 18070, 18071, 18072, 18073, 18074, 18075, 18076, 18077, 18078, 18079, 18080, 18081, 18082, 18083, 18084, 18085, 18086, 18087, 18088, 18089, 18090, 18091, 18092, 18093, 18094, 18095, 18096, 18097, 18098, 18099, 18100, 18101, 18102, 18103, 18104, 18105, 18106, 18107, 18108, 18109, 18110, 18111, 18112, 18113, 18114, 18115, 18116, 18117, 18118, 18119, 18120, 18121, 18122, 18123, 18124, 18125, 18126, 18127, 18128, 18129, 18130, 18131, 18132, 18133, 18134, 18135, 18136, 18137, 18138, 18139, 18140, 18141, 19766, 19767, 19768, 19769, 19770, 19771, 19772, 19773, 19774, 19775, 19776, 19777, 19778, 19779, 19780, 19781, 19782, 19783, 19784, 19785, 19786, 19787, 19788, 19789, 19790, 19791, 19792, 19793, 19794, 19795, 19796, 19797, 19798, 19799, 19800, 19801, 19802, 19803, 19804, 19805, 19806, 19807, 19808, 19809, 19810, 19811, 19812, 19813, 19814, 19815, 19816, 19817, 19818, 19819, 19820, 19821, 19822, 19823, 19824, 19825, 19826, 19827, 19828, 19829, 19830, 19831, 19832, 19833, 19834, 19835, 19836, 19837, 19838, 19839, 19840, 19841, 19842, 19843, 19844, 19845, 19846, 19847, 19848, 19849, 19850, 19851, 19852, 19853, 19854, 19855, 19856, 19857, 19858, 19859, 19860, 19861, 19862, 19863, 19864, 19865, 19866, 19867, 19868, 19869, 19870, 19871, 19872, 19873, 19874, 19875, 19876, 19877, 19878, 19879, 19880, 19881, 19882, 19883, 19884, 19885, 19886, 19887, 19888, 19889, 19890, 19891, 19892, 19893, 19894, 19895, 19896, 19897, 19898, 19899, 19900, 19901, 19902, 19903, 19904, 19905, 19906, 19907, 19908, 19909, 19910, 19911, 19912, 19913, 19914, 19915, 19916, 19917, 19918, 19919, 19920, 19921, 19922, 19923, 19924, 19925, 19926, 19927, 19928, 19929, 19930, 19931, 19932, 19933, 19934, 19935, 19936, 19937, 19938, 19939, 19940, 19941, 19942, 19943, 19944, 19945, 19946, 19947, 19948, 19949, 19950, 19951, 19952, 19953, 19954, 19955, 19956, 19957, 19958, 19959, 19960, 19961, 19962, 19963, 19964, 19965, 19966, 19967, 19968, 19969, 19970, 19971, 19972, 19973, 19974, 19975, 19976, 19977, 19978, 19979, 19980, 19981, 19982, 19983, 19984, 19985, 19986, 19987, 19988, 19989, 19990, 19991, 19992, 19993, 19994, 19995, 19996, 19997, 19998, 19999, 20000, 20001, 20002, 20003, 20004, 20005, 20006, 20007, 20008, 20009, 20010, 20011, 20012, 20013, 20014, 20015, 20016, 20017, 20018, 20019, 20020, 20021, 20022, 20023, 20024, 20025, 20026, 20027, 20028, 20029, 20030, 20031, 20032, 20033, 20034, 20035, 20036, 20037, 20038, 20039, 20040, 20041, 20042, 20043, 20044, 20045, 20046, 20047, 20048, 20049, 20050, 20051, 20052, 20053, 20054, 20055, 20056, 20057, 20058, 20059, 20060, 20061, 20062, 20063, 20064, 20065, 20066, 20067, 20068, 20069, 20070, 20071, 20072, 20073, 20074, 20075, 20076, 20077, 20078, 20079, 20080, 20081, 20082, 20083, 20084, 20085, 20086, 20087, 20088, 20089, 20090, 20091, 20092, 20093, 20094, 20095, 20096, 20097, 20098, 20099, 20100, 20101, 20102, 20103, 20104, 20105, 20106, 20107, 20108, 20109, 20110, 20111, 20112, 20113, 20114, 20115, 20116, 20117, 20118, 20119, 20120, 20121, 20122, 20123, 20124, 20125, 20126, 20127, 20128, 20129, 20130, 20131, 20132, 20133, 20134, 20135, 20136, 20137, 20138, 20139, 20140, 20141, 20142, 20143, 20144, 20145, 20146, 20147, 20148, 20149, 20150, 20151, 20152, 20153, 20154, 20155, 20156, 20157, 20158, 20159, 20160, 20161, 20162, 20163, 20164, 20165, 20166, 20167, 20168, 20169, 20170, 20171, 20172, 20173, 20174, 20175, 20176, 20177, 20178, 20179, 20180, 20181, 20182, 20183, 20184, 20185, 20186, 20187, 20188, 20189, 20190, 20191, 20192, 20193, 20194, 20195, 20196, 20197, 20198, 20199, 20200, 20201, 20202, 20203, 20204, 20205, 20206, 20207, 20208, 20209, 20210, 20211, 20212, 20213, 20214, 20215, 20216, 20217, 20218, 20219, 20220, 20221, 20222, 20223, 20224, 20225, 20226, 20227, 20228, 20229, 20230, 20231, 20232, 20233, 20234, 20235, 20236, 20237, 20238, 20239, 20240, 20241, 20242, 20243, 20244, 20245, 20246, 20247, 20248, 20249, 20250, 20251, 20252, 20253, 20254, 20255, 20256, 20257, 20258, 20259, 20260, 20261, 20262, 20263, 20264, 20265, 20266, 20267, 20268, 20269, 20270, 20271, 20272, 20273, 20274, 20275, 20276, 20277, 20278, 20279, 20280, 20281, 20282, 20283, 20284, 20285, 20286, 20287, 20288, 20289, 20290, 20291, 20292, 20293, 20294, 20295, 20296, 20297, 20298, 20299, 20300, 20301, 20302, 20303, 20304, 20305, 20306, 20307, 20308, 20309, 20310, 20311, 20312, 20313, 20314, 20315, 20316, 20317, 20318, 20319, 20320, 20321, 20322, 20323, 20324, 20325, 20326, 20327, 20328, 20329, 20330, 20331, 20332, 20333, 20334, 20335, 20336, 20337, 20338, 20339, 20340, 20341, 20342, 20343, 20344, 20345, 20346, 20347, 20348, 20349, 20350, 20351, 20352, 20353, 20354, 20355, 20356, 20357, 20358, 20359, 20360, 20361, 20362, 20363, 20364, 20365, 20366, 20367, 20368, 20369, 20370, 20371, 20372, 20373, 20374, 20375, 20376, 20377, 20378, 20379, 20380, 20381, 20382, 20383, 20384, 20385, 20386, 20387, 20388, 20389, 20390, 20391, 20392, 20393, 20394, 20395, 20396, 20397, 20398, 20399, 20400, 20401, 20402, 20403, 20404, 20405, 20406, 20407, 20408, 20409, 20410, 20411, 20412, 20413, 20414, 20415, 20416, 20417, 20418, 20419, 20420, 20421, 20422, 20423, 20424, 20425, 20426, 20427, 20428, 20429, 20430, 20431, 20432, 20433, 20434, 20435, 20436, 20437, 20438, 20439, 20440, 20441, 20442, 20443, 20444, 20445, 20446, 20447, 20448, 20449, 20450, 20451, 20452, 20453, 20454, 20455, 20456, 20457, 20458, 20459, 20460, 20461, 20462, 20463, 20464, 20465, 20466, 20467, 20468, 20469, 20470, 20471, 20472, 20473, 20474, 20475, 20476, 20477, 20478, 20479, 20480, 20481, 20482, 20483, 20484, 20485, 20486, 20487, 20488, 20489, 20490, 20491, 20492, 20493, 20494, 20495, 20496, 20497, 20498, 20499, 20500, 20501, 20502, 20503, 20504, 20505, 20506, 20507, 20508, 20509, 20510, 20511, 20512, 20513, 20514, 20515, 20516, 20517, 20518, 20519, 20520, 20521, 20522, 20523, 20524, 20525, 20526, 20527, 20528, 20529, 20530, 20531, 20532, 20533, 20534, 20535, 20536, 20537, 20538, 20539, 20540, 20541, 20542, 20543, 20544, 20545, 20546, 20547, 20548, 20549, 20550, 20551, 20552, 20553, 20554, 20555, 20556, 20557, 20558, 20559, 20560, 20561, 20562, 20563, 20564, 20565, 20566, 20567, 20568, 20569, 20570, 20571, 20572, 20573, 20574, 20575, 20576, 20577, 20578, 20579, 20580, 20581, 20582, 20583, 20584, 20585, 20586, 20587, 20588, 20589, 20590, 20591, 20592, 20593, 20594, 20595, 20596, 20597, 20598, 20599, 20600, 20601, 20602, 20603, 20604, 20605, 20606, 20607, 20608, 20609, 20610, 20611, 20612, 20613, 20614, 20615, 20616, 20617, 20618, 20619, 20620, 20621, 20622, 20623, 20624, 20625, 20626, 20627, 20628, 20629, 20630, 20631, 20632, 20633, 20634, 20635, 20636, 20637, 20638, 20639, 20640, 20641, 20642, 20643, 20644, 20645, 20646, 20647, 20648, 20649, 20650, 20651, 20652, 20653, 20654, 20655, 20656, 20657, 20658, 20659, 20660, 20661, 20662, 20663, 20664, 20665, 20666, 20667, 20668, 20669, 20670, 20671, 20672, 20673, 20674, 20675, 20676, 20677, 20678, 20679, 20680, 20681, 20682, 20683, 20684, 20685, 20686, 20687, 20688, 20689, 20690, 20691, 20692, 20693, 20694, 20695, 20696, 20697, 20698, 20699, 20700, 20701, 20702, 20703, 20704, 20705, 20706, 20707, 20708, 20709, 20710, 20711, 20712, 20713, 20714, 20715, 20716, 20717, 20718, 20719, 20720, 20721, 20722, 20723, 20724, 20725, 20726, 20727, 20728, 20729, 20730, 20731, 20732, 20733, 20734, 20735, 20736, 20737, 20738, 20739, 20740, 20741, 20742, 20743, 20744, 20745, 20746, 20747, 20748, 20749, 20750, 20751, 20752, 20753, 20754, 20755, 20756, 20757, 20758, 20759, 20760, 20761, 20762, 20763, 20764, 20765, 20766, 20767, 20768, 20769, 20770, 20771, 20772, 20773, 20774, 20775, 20776, 20777, 20778, 20779, 20780, 20781, 20782, 20783, 20784, 20785, 20786, 20787, 20788, 20789, 20790, 20791, 20792, 20793, 20794, 20795, 20796, 20797, 20798, 20799, 20800, 20801, 20802, 20803, 20804, 20805, 20806, 20807, 20808, 20809, 20810, 20811, 20812, 20813, 20814, 20815, 20816, 20817, 20818, 20819, or 20820, and wherein the [module (a)+module (b)] protein or peptide, the at least one module (c), and the at least one compound (d) are linked to each other in any arrangement and stoichiometry.

In a preferred embodiment of the second aspect, the present invention relates to a delivery system for delivery of a compound into a cell comprising or consisting of at least one conjugate comprising, essentially consisting of or consisting of:

(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
wherein the at least one module (b) and the at least one module (c) are comprised or contained within a [module (b)+module (c)] protein or peptide, and wherein the at least one module (a), the [module (b)+module (c)] protein or peptide, and the at least one compound (d) are linked to each other in any arrangement. The conjugates of the present invention optionally comprise a nuclear localization signal.

Preferably, the [module (b)+module (c)] protein or peptide comprises, consists essentially of, consists of or contains a CX1a peptide (SEQ ID NO: 2) (bc1), a CX2a peptide (SEQ ID NO: 3) (bc2), a peptide comprising an amino acid sequence comprising SEQ ID NO: 4 (bc3), a reduced toxicity or non-toxic toxin A-subunit comprising a module (b) protein or peptide (bc4), a reduced toxicity or non-toxic cholera toxin A-subunit (bc5), a reduced toxicity or non-toxic LT A-subunit (bc6), a reduced toxicity or non-toxic LT-II A-subunit (bc7), a reduced toxicity or non-toxic Pseudomonas exotoxin A (bc8), or an AChE protein or peptide comprising an amino acid sequence selected from the group consisting SEQ ID NO: 292 (bc9), SEQ ID NO: 293 (bc10), SEQ ID NO: 294 (bell), SEQ ID NO: 295 (bc12), SEQ ID NO: 296 (bc13), SEQ ID NO: 297 (bc14), SEQ ID NO: 298 (bc15), SEQ ID NO: 299 (bc16), SEQ ID NO: 300 (bc17), SEQ ID NO: 301 (bc18), SEQ ID NO: 302 (bc19), SEQ ID NO: 303 (bc20), and SEQ ID NO: 304 (bc21).

Thus, a preferred conjugate of the present invention comprises, essentially consists of, or consists of or contains:

(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
wherein the at least one module (b) and the at least one module (c) are comprised or contained within a [module (b)+module (c)] protein or peptide that comprises, consists essentially of, consists of or contains bc1, bc2, bc3, bc4, bc5, bc6, bc7, bc8, bc9, bc10, bell, bc12, bc13, bc14, be 15, be 16, be 17, be 18, be 19, bc20, or bc21, and wherein the at least one module (a), the [module (b)+module (c)] protein or peptide, and the at least one compound (d) are arranged in any arrangement and in any stoichiometry. The conjugates of the present invention optionally comprise a nuclear localization signal.

Another preferred conjugate of the present invention comprises, essentially consists of, or consists of or contains:

(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
wherein the at least one module (b) and the at least one module (c) are comprised or contained within a [module (b)+module (c)] protein or peptide, wherein the [module (b)+module (c)] protein or peptide and the at least one module (a) are selected from the group of combinations consisting of bc1+a1 (K4359), bc1+a2 (K4360), bc1+a3 (K4361), bc1+a4 (K4362), bc1+a5 (K4363), bc1+a6 (K4364), bc1+a7 (K4365), bc1+a8 (K4366), bc1+a9 (K4367), bc1+a10 (K4368), bc1+a11 (K4369), bc1+a12 (K4370), bc1+a13 (K4371), bc1+a14 (K4372), bc1+a15 (K4373), bc1+a16 (K4374), bc1+a17 (K4375), bc1+a18 (K4376), bc1+a19 (K4377), bc1+a20 (K4378), bc1+a21 (K4379), bc1+a22 (K4380), bc1+a23 (K4381), bc1+a24 (K4382), bc1+a25 (K4383), bc1+a26 (K4384), bc1+a27 (K4385), bc1+a28 (K4386), bc1+a29 (K4387), bc1+a30 (K4388), bc1+a31 (K4389), bc1+a32 (K4390), bc1+a33 (K4391), bc1+a34 (K4392), bc1+a35 (K4393), bc1+a36 (K4394), bc1+a37 (K4395), bc1+a38 (K4396), bc1+a39 (K4397), bc1+a40 (K4398), bc1+a41 (K4399), bc1+a42 (K4400), bc1+a43 (K4401), bc1+a44 (K4402), bc1+a45 (K4403), bc1+a46 (K4404), bc1+a47 (K4405), bc1+a48 (K4406), bc1+a49 (K4407), bc1+a50 (K4408), bc1+a51 (K4409), bc1+a52 (K4410), bc1+a53 (K4411), bc1+a54 (K4412), bc1+a55 (K4413), bc1+a56 (K4414), bc1+a57 (K4415), bc1+a58 (K4416), bc1+a59 (K4417), bc1+a60 (K4418), bc1+a61 (K4419), bc1+a62 (K4420), bc1+a63 (K4421), bc1+a64 (K4422), bc1+a65 (K4423), bc1+a66 (K4424), bc1+a67 (K4425), bc1+a68 (K4426), bc1+a69 (K4427), bc1+a70 (K4428), bc2+a1 (K4429), bc2+a2 (K4430), bc2+a3 (K4431), bc2+a4 (K4432), bc2+a5 (K4433), bc2+a6 (K4434), bc2+a7 (K4435), bc2+a8 (K4436), bc2+a9 (K4437), bc2+a10 (K4438), bc2+a11 (K4439), bc2+a12 (K4440), bc2+a13 (K4441), bc2+a14 (K4442), bc2+a15 (K4443), bc2+a16 (K4444), bc2+a17 (K4445), bc2+a18 (K4446), bc2+a19 (K4447), bc2+a20 (K4448), bc2+a21 (K4449), bc2+a22 (K4450), bc2+a23 (K4451), bc2+a24 (K4452), bc2+a25 (K4453), bc2+a26 (K4454), bc2+a27 (K4455), bc2+a28 (K4456), bc2+a29 (K4457), bc2+a30 (K4458), bc2+a31 (K4459), bc2+a32 (K4460), bc2+a33 (K4461), bc2+a34 (K4462), bc2+a35 (K4463), bc2+a36 (K4464), bc2+a37 (K4465), bc2+a38 (K4466), bc2+a39 (K4467), bc2+a40 (K4468), bc2+a41 (K4469), bc2+a42 (K4470), bc2+a43 (K4471), bc2+a44 (K4472), bc2+a45 (K4473), bc2+a46 (K4474), bc2+a47 (K4475), bc2+a48 (K4476), bc2+a49 (K4477), bc2+a50 (K4478), bc2+a51 (K4479), bc2+a52 (K4480), bc2+a53 (K4481), bc2+a54 (K4482), bc2+a55 (K4483), bc2+a56 (K4484), bc2+a57 (K4485), bc2+a58 (K4486), bc2+a59 (K4487), bc2+a60 (K4488), bc2+a61 (K4489), bc2+a62 (K4490), bc2+a63 (K4491), bc2+a64 (K4492), bc2+a65 (K4493), bc2+a66 (K4494), bc2+a67 (K4495), bc2+a68 (K4496), bc2+a69 (K4497), bc2+a70 (K4498), bc3+a1 (K4499), bc3+a2 (K4500), bc3+a3 (K4501), bc3+a4 (K4502), bc3+a5 (K4503), bc3+a6 (K4504), bc3+a7 (K4505), bc3+a8 (K4506), bc3+a9 (K4507), bc3+a10 (K4508), bc3+a11 (K4509), bc3+a12 (K4510), bc3+a13 (K4511), bc3+a14 (K4512), bc3+a15 (K4513), bc3+a16 (K4514), bc3+a17 (K4515), bc3+a18 (K4516), bc3+a19 (K4517), bc3+a20 (K4518), bc3+a21 (K4519), bc3+a22 (K4520), bc3+a23 (K4521), bc3+a24 (K4522), bc3+a25 (K4523), bc3+a26 (K4524), bc3+a27 (K4525), bc3+a28 (K4526), bc3+a29 (K4527), bc3+a30 (K4528), bc3+a31 (K4529), bc3+a32 (K4530), bc3+a33 (K4531), bc3+a34 (K4532), bc3+a35 (K4533), bc3+a36 (K4534), bc3+a37 (K4535), bc3+a38 (K4536), bc3+a39 (K4537), bc3+a40 (K4538), bc3+a41 (K4539), bc3+a42 (K4540), bc3+a43 (K4541), bc3+a44 (K4542), bc3+a45 (K4543), bc3+a46 (K4544), bc3+a47 (K4545), bc3+a48 (K4546), bc3+a49 (K4547), bc3+a50 (K4548), bc3+a51 (K4549), bc3+a52 (K4550), bc3+a53 (K4551), bc3+a54 (K4552), bc3+a55 (K4553), bc3+a56 (K4554), bc3+a57 (K4555), bc3+a58 (K4556), bc3+a59 (K4557), bc3+a60 (K4558), bc3+a61 (K4559), bc3+a62 (K4560), bc3+a63 (K4561), bc3+a64 (K4562), bc3+a65 (K4563), bc3+a66 (K4564), bc3+a67 (K4565), bc3+a68 (K4566), bc3+a69 (K4567), bc3+a70 (K4568), bc4+a1 (K4569), bc4+a2 (K4570), bc4+a3 (K4571), bc4+a4 (K4572), bc4+a5 (K4573), bc4+a6 (K4574), bc4+a7 (K4575), bc4+a8 (K4576), bc4+a9 (K4577), bc4+a10 (K4578), bc4+a11 (K4579), bc4+a12 (K4580), bc4+a13 (K4581), bc4+a14 (K4582), bc4+a15 (K4583), bc4+a16 (K4584), bc4+a17 (K4585), bc4+a18 (K4586), bc4+a19 (K4587), bc4+a20 (K4588), bc4+a21 (K4589), bc4+a22 (K4590), bc4+a23 (K4591), bc4+a24 (K4592), bc4+a25 (K4593), bc4+a26 (K4594), bc4+a27 (K4595), bc4+a28 (K4596), bc4+a29 (K4597), bc4+a30 (K4598), bc4+a31 (K4599), bc4+a32 (K4600), bc4+a33 (K4601), bc4+a34 (K4602), bc4+a35 (K4603), bc4+a36 (K4604), bc4+a37 (K4605), bc4+a38 (K4606), bc4+a39 (K4607), bc4+a40 (K4608), bc4+a41 (K4609), bc4+a42 (K4610), bc4+a43 (K4611), bc4+a44 (K4612), bc4+a45 (K4613), bc4+a46 (K4614), bc4+a47 (K4615), bc4+a48 (K4616), bc4+a49 (K4617), bc4+a50 (K4618), bc4+a51 (K4619), bc4+a52 (K4620), bc4+a53 (K4621), bc4+a54 (K4622), bc4+a55 (K4623), bc4+a56 (K4624), bc4+a57 (K4625), bc4+a58 (K4626), bc4+a59 (K4627), bc4+a60 (K4628), bc4+a61 (K4629), bc4+a62 (K4630), bc4+a63 (K4631), bc4+a64 (K4632), bc4+a65 (K4633), bc4+a66 (K4634), bc4+a67 (K4635), bc4+a68 (K4636), bc4+a69 (K4637), bc4+a70 (K4638), bc5+a1 (K4639), bc5+a2 (K4640), bc5+a3 (K4641), bc5+a4 (K4642), bc5+a5 (K4643), bc5+a6 (K4644), bc5+a7 (K4645), bc5+a8 (K4646), bc5+a9 (K4647), bc5+a10 (K4648), bc5+a11 (K4649), bc5+a12 (K4650), bc5+a13 (K4651), bc5+a14 (K4652), bc5+a15 (K4653), bc5+a16 (K4654), bc5+a17 (K4655), bc5+a18 (K4656), bc5+a19 (K4657), bc5+a20 (K4658), bc5+a21 (K4659), bc5+a22 (K4660), bc5+a23 (K4661), bc5+a24 (K4662), bc5+a25 (K4663), bc5+a26 (K4664), bc5+a27 (K4665), bc5+a28 (K4666), bc5+a29 (K4667), bc5+a30 (K4668), bc5+a31 (K4669), bc5+a32 (K4670), bc5+a33 (K4671), bc5+a34 (K4672), bc5+a35 (K4673), bc5+a36 (K4674), bc5+a37 (K4675), bc5+a38 (K4676), bc5+a39 (K4677), bc5+a40 (K4678), bc5+a41 (K4679), bc5+a42 (K4680), bc5+a43 (K4681), bc5+a44 (K4682), bc5+a45 (K4683), bc5+a46 (K4684), bc5+a47 (K4685), bc5+a48 (K4686), bc5+a49 (K4687), bc5+a50 (K4688), bc5+a51 (K4689), bc5+a52 (K4690), bc5+a53 (K4691), bc5+a54 (K4692), bc5+a55 (K4693), bc5+a56 (K4694), bc5+a57 (K4695), bc5+a58 (K4696), bc5+a59 (K4697), bc5+a60 (K4698), bc5+a61 (K4699), bc5+a62 (K4700), bc5+a63 (K4701), bc5+a64 (K4702), bc5+a65 (K4703), bc5+a66 (K4704), bc5+a67 (K4705), bc5+a68 (K4706), bc5+a69 (K4707), bc5+a70 (K4708), bc6+a1 (K4709), bc6+a2 (K4710), bc6+a3 (K4711), bc6+a4 (K4712), bc6+a5 (K4713), bc6+a6 (K4714), bc6+a7 (K4715), bc6+a8 (K4716), bc6+a9 (K4717), bc6+a10 (K4718), bc6+a11 (K4719), bc6+a12 (K4720), bc6+a13 (K4721), bc6+a14 (K4722), bc6+a15 (K4723), bc6+a16 (K4724), bc6+a17 (K4725), bc6+a18 (K4726), bc6+a19 (K4727), bc6+a20 (K4728), bc6+a21 (K4729), bc6+a22 (K4730), bc6+a23 (K4731), bc6+a24 (K4732), bc6+a25 (K4733), bc6+a26 (K4734), bc6+a27 (K4735), bc6+a28 (K4736), bc6+a29 (K4737), bc6+a30 (K4738), bc6+a31 (K4739), bc6+a32 (K4740), bc6+a33 (K4741), bc6+a34 (K4742), bc6+a35 (K4743), bc6+a36 (K4744), bc6+a37 (K4745), bc6+a38 (K4746), bc6+a39 (K4747), bc6+a40 (K4748), bc6+a41 (K4749), bc6+a42 (K4750), bc6+a43 (K4751), bc6+a44 (K4752), bc6+a45 (K4753), bc6+a46 (K4754), bc6+a47 (K4755), bc6+a48 (K4756), bc6+a49 (K4757), bc6+a50 (K4758), bc6+a51 (K4759), bc6+a52 (K4760), bc6+a53 (K4761), bc6+a54 (K4762), bc6+a55 (K4763), bc6+a56 (K4764), bc6+a57 (K4765), bc6+a58 (K4766), bc6+a59 (K4767), bc6+a60 (K4768), bc6+a61 (K4769), bc6+a62 (K4770), bc6+a63 (K4771), bc6+a64 (K4772), bc6+a65 (K4773), bc6+a66 (K4774), bc6+a67 (K4775), bc6+a68 (K4776), bc6+a69 (K4777), bc6+a70 (K4778), bc7+a1 (K4779), bc7+a2 (K4780), bc7+a3 (K4781), bc7+a4 (K4782), bc7+a5 (K4783), bc7+a6 (K4784), bc7+a7 (K4785), bc7+a8 (K4786), bc7+a9 (K4787), bc7+a10 (K4788), bc7+a11 (K4789), bc7+a12 (K4790), bc7+a13 (K4791), bc7+a14 (K4792), bc7+a15 (K4793), bc7+a16 (K4794), bc7+a17 (K4795), bc7+a18 (K4796), bc7+a19 (K4797), bc7+a20 (K4798), bc7+a21 (K4799), bc7+a22 (K4800), bc7+a23 (K4801), bc7+a24 (K4802), bc7+a25 (K4803), bc7+a26 (K4804), bc7+a27 (K4805), bc7+a28 (K4806), bc7+a29 (K4807), bc7+a30 (K4808), bc7+a31 (K4809), bc7+a32 (K4810), bc7+a33 (K4811), bc7+a34 (K4812), bc7+a35 (K4813), bc7+a36 (K4814), bc7+a37 (K4815), bc7+a38 (K4816), bc7+a39 (K4817), bc7+a40 (K4818), bc7+a41 (K4819), bc7+a42 (K4820), bc7+a43 (K4821), bc7+a44 (K4822), bc7+a45 (K4823), bc7+a46 (K4824), bc7+a47 (K4825), bc7+a48 (K4826), bc7+a49 (K4827), bc7+a50 (K4828), bc7+a51 (K4829), bc7+a52 (K4830), bc7+a53 (K4831), bc7+a54 (K4832), bc7+a55 (K4833), bc7+a56 (K4834), bc7+a57 (K4835), bc7+a58 (K4836), bc7+a59 (K4837), bc7+a60 (K4838), bc7+a61 (K4839), bc7+a62 (K4840), bc7+a63 (K4841), bc7+a64 (K4842), bc7+a65 (K4843), bc7+a66 (K4844), bc7+a67 (K4845), bc7+a68 (K4846), bc7+a69 (K4847), bc7+a70 (K4848), bc8+a1 (K4849), bc8+a2 (K4850), bc8+a3 (K4851), bc8+a4 (K4852), bc8+a5 (K4853), bc8+a6 (K4854), bc8+a7 (K4855), bc8+a8 (K4856), bc8+a9 (K4857), bc8+a10 (K4858), bc8+a11 (K4859), bc8+a12 (K4860), bc8+a13 (K4861), bc8+a14 (K4862), bc8+a15 (K4863), bc8+a16 (K4864), bc8+a17 (K4865), bc8+a18 (K4866), bc8+a19 (K4867), bc8+a20 (K4868), bc8+a21 (K4869), bc8+a22 (K4870), bc8+a23 (K4871), bc8+a24 (K4872), bc8+a25 (K4873), bc8+a26 (K4874), bc8+a27 (K4875), bc8+a28 (K4876), bc8+a29 (K4877), bc8+a30 (K4878), bc8+a31 (K4879), bc8+a32 (K4880), bc8+a33 (K4881), bc8+a34 (K4882), bc8+a35 (K4883), bc8+a36 (K4884), bc8+a37 (K4885), bc8+a38 (K4886), bc8+a39 (K4887), bc8+a40 (K4888), bc8+a41 (K4889), bc8+a42 (K4890), bc8+a43 (K4891), bc8+a44 (K4892), bc8+a45 (K4893), bc8+a46 (K4894), bc8+a47 (K4895), bc8+a48 (K4896), bc8+a49 (K4897), bc8+a50 (K4898), bc8+a51 (K4899), bc8+a52 (K4900), bc8+a53 (K4901), bc8+a54 (K4902), bc8+a55 (K4903), bc8+a56 (K4904), bc8+a57 (K4905), bc8+a58 (K4906), bc8+a59 (K4907), bc8+a60 (K4908), bc8+a61 (K4909), bc8+a62 (K4910), bc8+a63 (K4911), bc8+a64 (K4912), bc8+a65 (K4913), bc8+a66 (K4914), bc8+a67 (K4915), bc8+a68 (K4916), bc8+a69 (K4917), bc8+a70 (K4918), bc1+a71 (K18142), bc1+a72 (K18143), bc1+a73 (K18144), bc1+a74 (K18145), bc1+a75 (K18146), bc1+a76 (K18147), bc1+a77 (K18148), bc1+a78 (K18149), bc1+a79 (K18150), bc1+a80 (K18151), bc1+a81 (K18152), bc1+a82 (K18153), bc1+a83 (K18154), bc1+a84 (K18155), bc1+a85 (K18156), bc1+a86 (K18157), bc1+a87 (K18158), bc1+a88 (K18159), bc1+a89 (K18160), bc1+a90 (K18161), bc1+a91 (K18162), bc1+a92 (K18163), bc1+a93 (K18164), bc1+a94 (K18165), bc1+a95 (K18166), bc1+a96 (K18167), bc1+a97 (K18168), bc1+a98 (K18169), bc1+a99 (K18170), bc1+a100 (K18171), bc1+a101 (K18172), bc1+a102 (K18173), bc1+a103 (K18174), bc1+a104 (K18175), bc2+a71 (K18176), bc2+a72 (K18177), bc2+a73 (K18178), bc2+a74 (K18179), bc2+a75 (K18180), bc2+a76 (K18181), bc2+a77 (K18182), bc2+a78 (K18183), bc2+a79 (K18184), bc2+a80 (K18185), bc2+a81 (K18186), bc2+a82 (K18187), bc2+a83 (K18188), bc2+a84 (K18189), bc2+a85 (K18190), bc2+a86 (K18191), bc2+a87 (K18192), bc2+a88 (K18193), bc2+a89 (K18194), bc2+a90 (K18195), bc2+a91 (K18196), bc2+a92 (K18197), bc2+a93 (K18198), bc2+a94 (K18199), bc2+a95 (K18200), bc2+a96 (K18201), bc2+a97 (K18202), bc2+a98 (K18203), bc2+a99 (K18204), bc2+a100 (K18205), bc2+a101 (K18206), bc2+a102 (K18207), bc2+a103 (K18208), bc2+a104 (K18209), bc3+a71 (K18210), bc3+a72 (K18211), bc3+a73 (K18212), bc3+a74 (K18213), bc3+a75 (K18214), bc3+a76 (K18215), bc3+a77 (K18216), bc3+a78 (K18217), bc3+a79 (K18218), bc3+a80 (K18219), bc3+a81 (K18220), bc3+a82 (K18221), bc3+a83 (K18222), bc3+a84 (K18223), bc3+a85 (K18224), bc3+a86 (K18225), bc3+a87 (K18226), bc3+a88 (K18227), bc3+a89 (K18228), bc3+a90 (K18229), bc3+a91 (K18230), bc3+a92 (K18231), bc3+a93 (K18232), bc3+a94 (K18233), bc3+a95 (K18234), bc3+a96 (K18235), bc3+a97 (K18236), bc3+a98 (K18237), bc3+a99 (K18238), bc3+a100 (K18239), bc3+a101 (K18240), bc3+a102 (K18241), bc3+a103 (K18242), bc3+a104 (K18243), bc4+a71 (K18244), bc4+a72 (K18245), bc4+a73 (K18246), bc4+a74 (K18247), bc4+a75 (K18248), bc4+a76 (K18249), bc4+a77 (K18250), bc4+a78 (K18251), bc4+a79 (K18252), bc4+a80 (K18253), bc4+a81 (K18254), bc4+a82 (K18255), bc4+a83 (K18256), bc4+a84 (K18257), bc4+a85 (K18258), bc4+a86 (K18259), bc4+a87 (K18260), bc4+a88 (K18261), bc4+a89 (K18262), bc4+a90 (K18263), bc4+a91 (K18264), bc4+a92 (K18265), bc4+a93 (K18266), bc4+a94 (K18267), bc4+a95 (K18268), bc4+a96 (K18269), bc4+a97 (K18270), bc4+a98 (K18271), bc4+a99 (K18272), bc4+a100 (K18273), bc4+a101 (K18274), bc4+a102 (K18275), bc4+a103 (K18276), bc4+a104 (K18277), bc5+a71 (K18278), bc5+a72 (K18279), bc5+a73 (K18280), bc5+a74 (K18281), bc5+a75 (K18282), bc5+a76 (K18283), bc5+a77 (K18284), bc5+a78 (K18285), bc5+a79 (K18286), bc5+a80 (K18287), bc5+a81 (K18288), bc5+a82 (K18289), bc5+a83 (K18290), bc5+a84 (K18291), bc5+a85 (K18292), bc5+a86 (K18293), bc5+a87 (K18294), bc5+a88 (K18295), bc5+a89 (K18296), bc5+a90 (K18297), bc5+a91 (K18298), bc5+a92 (K18299), bc5+a93 (K18300), bc5+a94 (K18301), bc5+a95 (K18302), bc5+a96 (K18303), bc5+a97 (K18304), bc5+a98 (K18305), bc5+a99 (K18306), bc5+a100 (K18307), bc5+a101 (K18308), bc5+a102 (K18309), bc5+a103 (K18310), bc5+a104 (K18311), bc6+a71 (K18312), bc6+a72 (K18313), bc6+a73 (K18314), bc6+a74 (K18315), bc6+a75 (K18316), bc6+a76 (K18317), bc6+a77 (K18318), bc6+a78 (K18319), bc6+a79 (K18320), bc6+a80 (K18321), bc6+a81 (K18322), bc6+a82 (K18323), bc6+a83 (K18324), bc6+a84 (K18325), bc6+a85 (K18326), bc6+a86 (K18327), bc6+a87 (K18328), bc6+a88 (K18329), bc6+a89 (K18330), bc6+a90 (K18331), bc6+a91 (K18332), bc6+a92 (K18333), bc6+a93 (K18334), bc6+a94 (K18335), bc6+a95 (K18336), bc6+a96 (K18337), bc6+a97 (K18338), bc6+a98 (K18339), bc6+a99 (K18340), bc6+a100 (K18341), bc6+a101 (K18342), bc6+a102 (K18343), bc6+a103 (K18344), bc6+a104 (K18345), bc7+a71 (K18346), bc7+a72 (K18347), bc7+a73 (K18348), bc7+a74 (K18349), bc7+a75 (K18350), bc7+a76 (K18351), bc7+a77 (K18352), bc7+a78 (K18353), bc7+a79 (K18354), bc7+a80 (K18355), bc7+a81 (K18356), bc7+a82 (K18357), bc7+a83 (K18358), bc7+a84 (K18359), bc7+a85 (K18360), bc7+a86 (K18361), bc7+a87 (K18362), bc7+a88 (K18363), bc7+a89 (K18364), bc7+a90 (K18365), bc7+a91 (K18366), bc7+a92 (K18367), bc7+a93 (K18368), bc7+a94 (K18369), bc7+a95 (K18370), bc7+a96 (K18371), bc7+a97 (K18372), bc7+a98 (K18373), bc7+a99 (K18374), bc7+a100 (K18375), bc7+a101 (K18376), bc7+a102 (K18377), bc7+a103 (K18378), bc7+a104 (K18379), bc8+a71 (K18380), bc8+a72 (K18381), bc8+a73 (K18382), bc8+a74 (K18383), bc8+a75 (K18384), bc8+a76 (K18385), bc8+a77 (K18386), bc8+a78 (K18387), bc8+a79 (K18388), bc8+a80 (K18389), bc8+a81 (K18390), bc8+a82 (K18391), bc8+a83 (K18392), bc8+a84 (K18393), bc8+a85 (K18394), bc8+a86 (K18395), bc8+a87 (K18396), bc8+a88 (K18397), bc8+a89 (K18398), bc8+a90 (K18399), bc8+a91 (K18400), bc8+a92 (K18401), bc8+a93 (K18402), bc8+a94 (K18403), bc8+a95 (K18404), bc8+a96 (K18405), bc8+a97 (K18406), bc8+a98 (K18407), bc8+a99 (K18408), bc8+a100 (K18409), bc8+a101 (K18410), bc8+a102 (K18411), bc8+a103 (K18412), bc8+a104 (K18413), bc9+a1 (K18414), bc9+a2 (K18415), bc9+a3 (K18416), bc9+a4 (K18417), bc9+a5 (K18418), bc9+a6 (K18419), bc9+a7 (K18420), bc9+a8 (K18421), bc9+a9 (K18422), bc9+a10 (K18423), bc9+a11 (K18424), bc9+a12 (K18425), bc9+a13 (K18426), bc9+a14 (K18427), bc9+a15 (K18428), bc9+a16 (K18429), bc9+a17 (K18430), bc9+a18 (K18431), bc9+a19 (K18432), bc9+a20 (K18433), bc9+a21 (K18434), bc9+a22 (K18435), bc9+a23 (K18436), bc9+a24 (K18437), bc9+a25 (K18438), bc9+a26 (K18439), bc9+a27 (K18440), bc9+a28 (K18441), bc9+a29 (K18442), bc9+a30 (K18443), bc9+a31 (K18444), bc9+a32 (K18445), bc9+a33 (K18446), bc9+a34 (K18447), bc9+a35 (K18448), bc9+a36 (K18449), bc9+a37 (K18450), bc9+a38 (K18451), bc9+a39 (K18452), bc9+a40 (K18453), bc9+a41 (K18454), bc9+a42 (K18455), bc9+a43 (K18456), bc9+a44 (K18457), bc9+a45 (K18458), bc9+a46 (K18459), bc9+a47 (K18460), bc9+a48 (K18461), bc9+a49 (K18462), bc9+a50 (K18463), bc9+a51 (K18464), bc9+a52 (K18465), bc9+a53 (K18466), bc9+a54 (K18467), bc9+a55 (K18468), bc9+a56 (K18469), bc9+a57 (K18470), bc9+a58 (K18471), bc9+a59 (K18472), bc9+a60 (K18473), bc9+a61 (K18474), bc9+a62 (K18475), bc9+a63 (K18476), bc9+a64 (K18477), bc9+a65 (K18478), bc9+a66 (K18479), bc9+a67 (K18480), bc9+a68 (K18481), bc9+a69 (K18482), bc9+a70 (K18483), bc9+a71 (K18484), bc9+a72 (K18485), bc9+a73 (K18486), bc9+a74 (K18487), bc9+a75 (K18488), bc9+a76 (K18489), bc9+a77 (K18490), bc9+a78 (K18491), bc9+a79 (K18492), bc9+a80 (K18493), bc9+a81 (K18494), bc9+a82 (K18495), bc9+a83 (K18496), bc9+a84 (K18497), bc9+a85 (K18498), bc9+a86 (K18499), bc9+a87 (K18500), bc9+a88 (K18501), bc9+a89 (K18502), bc9+a90 (K18503), bc9+a91 (K18504), bc9+a92 (K18505), bc9+a93 (K18506), bc9+a94 (K18507), bc9+a95 (K18508), bc9+a96 (K18509), bc9+a97 (K18510), bc9+a98 (K18511), bc9+a99 (K18512), bc9+a100 (K18513), bc9+a101 (K18514), bc9+a102 (K18515), bc9+a103 (K18516), bc9+a104 (K18517), bc10+a1 (K18518), bc10+a2 (K18519), bc10+a3 (K18520), bc10+a4 (K18521), bc10+a5 (K18522), bc10+a6 (K18523), bc10+a7 (K18524), bc10+a8 (K18525), bc10+a9 (K18526), bc10+a10 (K18527), bc10+a11 (K18528), bc10+a12 (K18529), bc10+a13 (K18530), bc10+a14 (K18531), bc10+a15 (K18532), bc10+a16 (K18533), bc10+a17 (K18534), bc10+a18 (K18535), bc10+a19 (K18536), bc10+a20 (K18537), bc10+a21 (K18538), bc10+a22 (K18539), bc10+a23 (K18540), bc10+a24 (K18541), bc10+a25 (K18542), bc10+a26 (K18543), bc10+a27 (K18544), bc10+a28 (K18545), bc10+a29 (K18546), bc10+a30 (K18547), bc10+a31 (K18548), bc10+a32 (K18549), bc10+a33 (K18550), bc10+a34 (K18551), bc10+a35 (K18552), bc10+a36 (K18553), bc10+a37 (K18554), bc10+a38 (K18555), bc10+a39 (K18556), bc10+a40 (K18557), bc10+a41 (K18558), bc10+a42 (K18559), bc10+a43 (K18560), bc10+a44 (K18561), bc10+a45 (K18562), bc10+a46 (K18563), bc10+a47 (K18564), bc10+a48 (K18565), bc10+a49 (K18566), bc10+a50 (K18567), bc10+a51 (K18568), bc10+a52 (K18569), bc10+a53 (K18570), bc10+a54 (K18571), bc10+a55 (K18572), bc10+a56 (K18573), bc10+a57 (K18574), bc10+a58 (K18575), bc10+a59 (K18576), bc10+a60 (K18577), bc10+a61 (K18578), bc10+a62 (K18579), bc10+a63 (K18580), bc10+a64 (K18581), bc10+a65 (K18582), bc10+a66 (K18583), bc10+a67 (K18584), bc10+a68 (K18585), bc10+a69 (K18586), bc10+a70 (K18587), bc10+a71 (K18588), bc10+a72 (K18589), bc10+a73 (K18590), bc10+a74 (K18591), bc10+a75 (K18592), bc10+a76 (K18593), bc10+a77 (K18594), bc10+a78 (K18595), bc10+a79 (K18596), bc10+a80 (K18597), bc10+a81 (K18598), bc10+a82 (K18599), bc10+a83 (K18600), bc10+a84 (K18601), bc10+a85 (K18602), bc10+a86 (K18603), bc10+a87 (K18604), bc10+a88 (K18605), bc10+a89 (K18606), bc10+a90 (K18607), bc10+a91 (K18608), bc10+a92 (K18609), bc10+a93 (K18610), bc10+a94 (K18611), bc10+a95 (K18612), bc10+a96 (K18613), bc10+a97 (K18614), bc10+a98 (K18615), bc10+a99 (K18616), bc10+a100 (K18617), bc10+a101 (K18618), bc10+a102 (K18619), bc10+a103 (K18620), bc10+a104 (K18621), bc11+a1 (K18622), bc11+a2 (K18623), bc11+a3 (K18624), bc11+a4 (K18625), bc11+a5 (K18626), bc11+a6 (K18627), bc11+a7 (K18628), bc11+a8 (K18629), bc11+a9 (K18630), bc11+a10 (K18631), bc11+a11 (K18632), bc11+a12 (K18633), bc11+a13 (K18634), bc11+a14 (K18635), bc11+a15 (K18636), bc11+a16 (K18637), bc11+a17 (K18638), bc11+a18 (K18639), bc11+a19 (K18640), bc11+a20 (K18641), bc11+a21 (K18642), bc11+a22 (K18643), bc11+a23 (K18644), bc11+a24 (K18645), bc11+a25 (K18646), bc11+a26 (K18647), bc11+a27 (K18648), bc11+a28 (K18649), bc11+a29 (K18650), bc11+a30 (K18651), bc11+a31 (K18652), bc11+a32 (K18653), bc11+a33 (K18654), bc11+a34 (K18655), bc11+a35 (K18656), bc11+a36 (K18657), bc11+a37 (K18658), bc11+a38 (K18659), bc11+a39 (K18660), bc11+a40 (K18661), bc11+a41 (K18662), bc11+a42 (K18663), bc11+a43 (K18664), bc11+a44 (K18665), bc11+a45 (K18666), bc11+a46 (K18667), bc11+a47 (K18668), bc11+a48 (K18669), bc11+a49 (K18670), bc11+a50 (K18671), bc11+a51 (K18672), bc11+a52 (K18673), bc11+a53 (K18674), bc11+a54 (K18675), bc11+a55 (K18676), bc11+a56 (K18677), bc11+a57 (K18678), bc11+a58 (K18679), bc11+a59 (K18680), bc11+a60 (K18681), bc11+a61 (K18682), bc11+a62 (K18683), bc11+a63 (K18684), bc11+a64 (K18685), bc11+a65 (K18686), bc11+a66 (K18687), bc11+a67 (K18688), bc11+a68 (K18689), bc11+a69 (K18690), bc11+a70 (K18691), bc11+a71 (K18692), bc11+a72 (K18693), bc11+a73 (K18694), bc11+a74 (K18695), bc11+a75 (K18696), bc11+a76 (K18697), bc11+a77 (K18698), bc11+a78 (K18699), bc11+a79 (K18700), bc11+a80 (K18701), bc11+a81 (K18702), bc11+a82 (K18703), bc11+a83 (K18704), bc11+a84 (K18705), bc11+a85 (K18706), bc11+a86 (K18707), bc11+a87 (K18708), bc11+a88 (K18709), bc11+a89 (K18710), bc11+a90 (K18711), bc11+a91 (K18712), bc11+a92 (K18713), bc11+a93 (K18714), bc11+a94 (K18715), bc11+a95 (K18716), bc11+a96 (K18717), bc11+a97 (K18718), bc11+a98 (K18719), bc11+a99 (K18720), bc11+a100 (K18721), bc11+a101 (K18722), bc11+a102 (K18723), bc11+a103 (K18724), bc11+a104 (K18725), bc12+a1 (K18726), bc12+a2 (K18727), bc12+a3 (K18728), bc12+a4 (K18729), bc12+a5 (K18730), bc12+a6 (K18731), bc12+a7 (K18732), bc12+a8 (K18733), bc12+a9 (K18734), bc12+a10 (K18735), bc12+a11 (K18736), bc12+a12 (K18737), bc12+a13 (K18738), bc12+a14 (K18739), bc12+a15 (K18740), bc12+a16 (K18741), bc12+a17 (K18742), bc12+a18 (K18743), bc12+a19 (K18744), bc12+a20 (K18745), bc12+a21 (K18746), bc12+a22 (K18747), bc12+a23 (K18748), bc12+a24 (K18749), bc12+a25 (K18750), bc12+a26 (K18751), bc12+a27 (K18752), bc12+a28 (K18753), bc12+a29 (K18754), bc12+a30 (K18755), bc12+a31 (K18756), bc12+a32 (K18757), bc12+a33 (K18758), bc12+a34 (K18759), bc12+a35 (K18760), bc12+a36 (K18761), bc12+a37 (K18762), bc12+a38 (K18763), bc12+a39 (K18764), bc12+a40 (K18765), bc12+a41 (K18766), bc12+a42 (K18767), bc12+a43 (K18768), bc12+a44 (K18769), bc12+a45 (K18770), bc12+a46 (K18771), bc12+a47 (K18772), bc12+a48 (K18773), bc12+a49 (K18774), bc12+a50 (K18775), bc12+a51 (K18776), bc12+a52 (K18777), bc12+a53 (K18778), bc12+a54 (K18779), bc12+a55 (K18780), bc12+a56 (K18781), bc12+a57 (K18782), bc12+a58 (K18783), bc12+a59 (K18784), bc12+a60 (K18785), bc12+a61 (K18786), bc12+a62 (K18787), bc12+a63 (K18788), bc12+a64 (K18789), bc12+a65 (K18790), bc12+a66 (K18791), bc12+a67 (K18792), bc12+a68 (K18793), bc12+a69 (K18794), bc12+a70 (K18795), bc12+a71 (K18796), bc12+a72 (K18797), bc12+a73 (K18798), bc12+a74 (K18799), bc12+a75 (K18800), bc12+a76 (K18801), bc12+a77 (K18802), bc12+a78 (K18803), bc12+a79 (K18804), bc12+a80 (K18805), bc12+a81 (K18806), bc12+a82 (K18807), bc12+a83 (K18808), bc12+a84 (K18809), bc12+a85 (K18810), bc12+a86 (K18811), bc12+a87 (K18812), bc12+a88 (K18813), bc12+a89 (K18814), bc12+a90 (K18815), bc12+a91 (K18816), bc12+a92 (K18817), bc12+a93 (K18818), bc12+a94 (K18819), bc12+a95 (K18820), bc12+a96 (K18821), bc12+a97 (K18822), bc12+a98 (K18823), bc12+a99 (K18824), bc12+a100 (K18825), bc12+a101 (K18826), bc12+a102 (K18827), bc12+a103 (K18828), bc12+a104 (K18829), bc13+a1 (K18830), bc13+a2 (K18831), bc13+a3 (K18832), bc13+a4 (K18833), bc13+a5 (K18834), bc13+a6 (K18835), bc13+a7 (K18836), bc13+a8 (K18837), bc13+a9 (K18838), bc13+a10 (K18839), bc13+a11 (K18840), bc13+a12 (K18841), bc13+a13 (K18842), bc13+a14 (K18843), bc13+a15 (K18844), bc13+a16 (K18845), bc13+a17 (K18846), bc13+a18 (K18847), bc13+a19 (K18848), bc13+a20 (K18849), bc13+a21 (K18850), bc13+a22 (K18851), bc13+a23 (K18852), bc13+a24 (K18853), bc13+a25 (K18854), bc13+a26 (K18855), bc13+a27 (K18856), bc13+a28 (K18857), bc13+a29 (K18858), bc13+a30 (K18859), bc13+a31 (K18860), bc13+a32 (K18861), bc13+a33 (K18862), bc13+a34 (K18863), bc13+a35 (K18864), bc13+a36 (K18865), bc13+a37 (K18866), bc13+a38 (K18867), bc13+a39 (K18868), bc13+a40 (K18869), bc13+a41 (K18870), bc13+a42 (K18871), bc13+a43 (K18872), bc13+a44 (K18873), bc13+a45 (K18874), bc13+a46 (K18875), bc13+a47 (K18876), bc13+a48 (K18877), bc13+a49 (K18878), bc13+a50 (K18879), bc13+a51 (K18880), bc13+a52 (K18881), bc13+a53 (K18882), bc13+a54 (K18883), bc13+a55 (K18884), bc13+a56 (K18885), bc13+a57 (K18886), bc13+a58 (K18887), bc13+a59 (K18888), bc13+a60 (K18889), bc13+a61 (K18890), bc13+a62 (K18891), bc13+a63 (K18892), bc13+a64 (K18893), bc13+a65 (K18894), bc13+a66 (K18895), bc13+a67 (K18896), bc13+a68 (K18897), bc13+a69 (K18898), bc13+a70 (K18899), bc13+a71 (K18900), bc13+a72 (K18901), bc13+a73 (K18902), bc13+a74 (K18903), bc13+a75 (K18904), bc13+a76 (K18905), bc13+a77 (K18906), bc13+a78 (K18907), bc13+a79 (K18908), bc13+a80 (K18909), bc13+a81 (K18910), bc13+a82 (K18911), bc13+a83 (K18912), bc13+a84 (K18913), bc13+a85 (K18914), bc13+a86 (K18915), bc13+a87 (K18916), bc13+a88 (K18917), bc13+a89 (K18918), bc13+a90 (K18919), bc13+a91 (K18920), bc13+a92 (K18921), bc13+a93 (K18922), bc13+a94 (K18923), bc13+a95 (K18924), bc13+a96 (K18925), bc13+a97 (K18926), bc13+a98 (K18927), bc13+a99 (K18928), bc13+a100 (K18929), bc13+a101 (K18930), bc13+a102 (K18931), bc13+a103 (K18932), bc13+a104 (K18933), bc14+a1 (K18934), bc14+a2 (K18935), bc14+a3 (K18936), bc14+a4 (K18937), bc14+a5 (K18938), bc14+a6 (K18939), bc14+a7 (K18940), bc14+a8 (K18941), bc14+a9 (K18942), bc14+a10 (K18943), bc14+a11 (K18944), bc14+a12 (K18945), bc14+a13 (K18946), bc14+a14 (K18947), bc14+a15 (K18948), bc14+a16 (K18949), bc14+a17 (K18950), bc14+a18 (K18951), bc14+a19 (K18952), bc14+a20 (K18953), bc14+a21 (K18954), bc14+a22 (K18955), bc14+a23 (K18956), bc14+a24 (K18957), bc14+a25 (K18958), bc14+a26 (K18959), bc14+a27 (K18960), bc14+a28 (K18961), bc14+a29 (K18962), bc14+a30 (K18963), bc14+a31 (K18964), bc14+a32 (K18965), bc14+a33 (K18966), bc14+a34 (K18967), bc14+a35 (K18968), bc14+a36 (K18969), bc14+a37 (K18970), bc14+a38 (K18971), bc14+a39 (K18972), bc14+a40 (K18973), bc14+a41 (K18974), bc14+a42 (K18975), bc14+a43 (K18976), bc14+a44 (K18977), bc14+a45 (K18978), bc14+a46 (K18979), bc14+a47 (K18980), bc14+a48 (K18981), bc14+a49 (K18982), bc14+a50 (K18983), bc14+a51 (K18984), bc14+a52 (K18985), bc14+a53 (K18986), bc14+a54 (K18987), bc14+a55 (K18988), bc14+a56 (K18989), bc14+a57 (K18990), bc14+a58 (K18991), bc14+a59 (K18992), bc14+a60 (K18993), bc14+a61 (K18994), bc14+a62 (K18995), bc14+a63 (K18996), bc14+a64 (K18997), bc14+a65 (K18998), bc14+a66 (K18999), bc14+a67 (K19000), bc14+a68 (K19001), bc14+a69 (K19002), bc14+a70 (K19003), bc14+a71 (K19004), bc14+a72 (K19005), bc14+a73 (K19006), bc14+a74 (K19007), bc14+a75 (K19008), bc14+a76 (K19009), bc14+a77 (K19010), bc14+a78 (K19011), bc14+a79 (K19012), bc14+a80 (K19013), bc14+a81 (K19014), bc14+a82 (K19015), bc14+a83 (K19016), bc14+a84 (K19017), bc14+a85 (K19018), bc14+a86 (K19019), bc14+a87 (K19020), bc14+a88 (K19021), bc14+a89 (K19022), bc14+a90 (K19023), bc14+a91 (K19024), bc14+a92 (K19025), bc14+a93 (K19026), bc14+a94 (K19027), bc14+a95 (K19028), bc14+a96 (K19029), bc14+a97 (K19030), bc14+a98 (K19031), bc14+a99 (K19032), bc14+a100 (K19033), bc14+a101 (K19034), bc14+a102 (K19035), bc14+a103 (K19036), bc14+a104 (K19037), bc15+a1 (K19038), bc15+a2 (K19039), bc15+a3 (K19040), bc15+a4 (K19041), bc15+a5 (K19042), bc15+a6 (K19043), bc15+a7 (K19044), bc15+a8 (K19045), bc15+a9 (K19046), bc15+a10 (K19047), bc15+a11 (K19048), bc15+a12 (K19049), bc15+a13 (K19050), bc15+a14 (K19051), bc15+a15 (K19052), bc15+a16 (K19053), bc15+a17 (K19054), bc15+a18 (K19055), bc15+a19 (K19056), bc15+a20 (K19057), bc15+a21 (K19058), bc15+a22 (K19059), bc15+a23 (K19060), bc15+a24 (K19061), bc15+a25 (K19062), bc15+a26 (K19063), bc15+a27 (K19064), bc15+a28 (K19065), bc15+a29 (K19066), bc15+a30 (K19067), bc15+a31 (K19068), bc15+a32 (K19069), bc15+a33 (K19070), bc15+a34 (K19071), bc15+a35 (K19072), bc15+a36 (K19073), bc15+a37 (K19074), bc15+a38 (K19075), bc15+a39 (K19076), bc15+a40 (K19077), bc15+a41 (K19078), bc15+a42 (K19079), bc15+a43 (K19080), bc15+a44 (K19081), bc15+a45 (K19082), bc15+a46 (K19083), bc15+a47 (K19084), bc15+a48 (K19085), bc15+a49 (K19086), bc15+a50 (K19087), bc15+a51 (K19088), bc15+a52 (K19089), bc15+a53 (K19090), bc15+a54 (K19091), bc15+a55 (K19092), bc15+a56 (K19093), bc15+a57 (K19094), bc15+a58 (K19095), bc15+a59 (K19096), bc15+a60 (K19097), bc15+a61 (K19098), bc15+a62 (K19099), bc15+a63 (K19100), bc15+a64 (K19101), bc15+a65 (K19102), bc15+a66 (K19103), bc15+a67 (K19104), bc15+a68 (K19105), bc15+a69 (K19106), bc15+a70 (K19107), bc15+a71 (K19108), bc15+a72 (K19109), bc15+a73 (K19110), bc15+a74 (K19111), bc15+a75 (K19112), bc15+a76 (K19113), bc15+a77 (K19114), bc15+a78 (K19115), bc15+a79 (K19116), bc15+a80 (K19117), bc15+a81 (K19118), bc15+a82 (K19119), bc15+a83 (K19120), bc15+a84 (K19121), bc15+a85 (K19122), bc15+a86 (K19123), bc15+a87 (K19124), bc15+a88 (K19125), bc15+a89 (K19126), bc15+a90 (K19127), bc15+a91 (K19128), bc15+a92 (K19129), bc15+a93 (K19130), bc15+a94 (K19131), bc15+a95 (K19132), bc15+a96 (K19133), bc15+a97 (K19134), bc15+a98 (K19135), bc15+a99 (K19136), bc15+a100 (K19137), bc15+a101 (K19138), bc15+a102 (K19139), bc15+a103 (K19140), bc15+a104 (K19141), bc16+a1 (K19142), bc16+a2 (K19143), bc16+a3 (K19144), bc16+a4 (K19145), bc16+a5 (K19146), bc16+a6 (K19147), bc16+a7 (K19148), bc16+a8 (K19149), bc16+a9 (K19150), bc16+a10 (K19151), bc16+a11 (K19152), bc16+a12 (K19153), bc16+a13 (K19154), bc16+a14 (K19155), bc16+a15 (K19156), bc16+a16 (K19157), bc16+a17 (K19158), bc16+a18 (K19159), bc16+a19 (K19160), bc16+a20 (K19161), bc16+a21 (K19162), bc16+a22 (K19163), bc16+a23 (K19164), bc16+a24 (K19165), bc16+a25 (K19166), bc16+a26 (K19167), bc16+a27 (K19168), bc16+a28 (K19169), bc16+a29 (K19170), bc16+a30 (K19171), bc16+a31 (K19172), bc16+a32 (K19173), bc16+a33 (K19174), bc16+a34 (K19175), bc16+a35 (K19176), bc16+a36 (K19177), bc16+a37 (K19178), bc16+a38 (K19179), bc16+a39 (K19180), bc16+a40 (K19181), bc16+a41 (K19182), bc16+a42 (K19183), bc16+a43 (K19184), bc16+a44 (K19185), bc16+a45 (K19186), bc16+a46 (K19187), bc16+a47 (K19188), bc16+a48 (K19189), bc16+a49 (K19190), bc16+a50 (K19191), bc16+a51 (K19192), bc16+a52 (K19193), bc16+a53 (K19194), bc16+a54 (K19195), bc16+a55 (K19196), bc16+a56 (K19197), bc16+a57 (K19198), bc16+a58 (K19199), bc16+a59 (K19200), bc16+a60 (K19201), bc16+a61 (K19202), bc16+a62 (K19203), bc16+a63 (K19204), bc16+a64 (K19205), bc16+a65 (K19206), bc16+a66 (K19207), bc16+a67 (K19208), bc16+a68 (K19209), bc16+a69 (K19210), bc16+a70 (K19211), bc16+a71 (K19212), bc16+a72 (K19213), bc16+a73 (K19214), bc16+a74 (K19215), bc16+a75 (K19216), bc16+a76 (K19217), bc16+a77 (K19218), bc16+a78 (K19219), bc16+a79 (K19220), bc16+a80 (K19221), bc16+a81 (K19222), bc16+a82 (K19223), bc16+a83 (K19224), bc16+a84 (K19225), bc16+a85 (K19226), bc16+a86 (K19227), bc16+a87 (K19228), bc16+a88 (K19229), bc16+a89 (K19230), bc16+a90 (K19231), bc16+a91 (K19232), bc16+a92 (K19233), bc16+a93 (K19234), bc16+a94 (K19235), bc16+a95 (K19236), bc16+a96 (K19237), bc16+a97 (K19238), bc16+a98 (K19239), bc16+a99 (K19240), bc16+a100 (K19241), bc16+a101 (K19242), bc16+a102 (K19243), bc16+a103 (K19244), bc16+a104 (K19245), bc17+a1 (K19246), bc17+a2 (K19247), bc17+a3 (K19248), bc17+a4 (K19249), bc17+a5 (K19250), bc17+a6 (K19251), bc17+a7 (K19252), bc17+a8 (K19253), bc17+a9 (K19254), bc17+a10 (K19255), bc17+a11 (K19256), bc17+a12 (K19257), bc17+a13 (K19258), bc17+a14 (K19259), bc17+a15 (K19260), bc17+a16 (K19261), bc17+a17 (K19262), bc17+a18 (K19263), bc17+a19 (K19264), bc17+a20 (K19265), bc17+a21 (K19266), bc17+a22 (K19267), bc17+a23 (K19268), bc17+a24 (K19269), bc17+a25 (K19270), bc17+a26 (K19271), bc17+a27 (K19272), bc17+a28 (K19273), bc17+a29 (K19274), bc17+a30 (K19275), bc17+a31 (K19276), bc17+a32 (K19277), bc17+a33 (K19278), bc17+a34 (K19279), bc17+a35 (K19280), bc17+a36 (K19281), bc17+a37 (K19282), bc17+a38 (K19283), bc17+a39 (K19284), bc17+a40 (K19285), bc17+a41 (K19286), bc17+a42 (K19287), bc17+a43 (K19288), bc17+a44 (K19289), bc17+a45 (K19290), bc17+a46 (K19291), bc17+a47 (K19292), bc17+a48 (K19293), bc17+a49 (K19294), bc17+a50 (K19295), bc17+a51 (K19296), bc17+a52 (K19297), bc17+a53 (K19298), bc17+a54 (K19299), bc17+a55 (K19300), bc17+a56 (K19301), bc17+a57 (K19302), bc17+a58 (K19303), bc17+a59 (K19304), bc17+a60 (K19305), bc17+a61 (K19306), bc17+a62 (K19307), bc17+a63 (K19308), bc17+a64 (K19309), bc17+a65 (K19310), bc17+a66 (K19311), bc17+a67 (K19312), bc17+a68 (K19313), bc17+a69 (K19314), bc17+a70 (K19315), bc17+a71 (K19316), bc17+a72 (K19317), bc17+a73 (K19318), bc17+a74 (K19319), bc17+a75 (K19320), bc17+a76 (K19321), bc17+a77 (K19322), bc17+a78 (K19323), bc17+a79 (K19324), bc17+a80 (K19325), bc17+a81 (K19326), bc17+a82 (K19327), bc17+a83 (K19328), bc17+a84 (K19329), bc17+a85 (K19330), bc17+a86 (K19331), bc17+a87 (K19332), bc17+a88 (K19333), bc17+a89 (K19334), bc17+a90 (K19335), bc17+a91 (K19336), bc17+a92 (K19337), bc17+a93 (K19338), bc17+a94 (K19339), bc17+a95 (K19340), bc17+a96 (K19341), bc17+a97 (K19342), bc17+a98 (K19343), bc17+a99 (K19344), bc17+a100 (K19345), bc17+a101 (K19346), bc17+a102 (K19347), bc17+a103 (K19348), bc17+a104 (K19349), bc18+a1 (K19350), bc18+a2 (K19351), bc18+a3 (K19352), bc18+a4 (K19353), bc18+a5 (K19354), bc18+a6 (K19355), bc18+a7 (K19356), bc18+a8 (K19357), bc18+a9 (K19358), bc18+a10 (K19359), bc18+a11 (K19360), bc18+a12 (K19361), bc18+a13 (K19362), bc18+a14 (K19363), bc18+a15 (K19364), bc18+a16 (K19365), bc18+a17 (K19366), bc18+a18 (K19367), bc18+a19 (K19368), bc18+a20 (K19369), bc18+a21 (K19370), bc18+a22 (K19371), bc18+a23 (K19372), bc18+a24 (K19373), bc18+a25 (K19374), bc18+a26 (K19375), bc18+a27 (K19376), bc18+a28 (K19377), bc18+a29 (K19378), bc18+a30 (K19379), bc18+a31 (K19380), bc18+a32 (K19381), bc18+a33 (K19382), bc18+a34 (K19383), bc18+a35 (K19384), bc18+a36 (K19385), bc18+a37 (K19386), bc18+a38 (K19387), bc18+a39 (K19388), bc18+a40 (K19389), bc18+a41 (K19390), bc18+a42 (K19391), bc18+a43 (K19392), bc18+a44 (K19393), bc18+a45 (K19394), bc18+a46 (K19395), bc18+a47 (K19396), bc18+a48 (K19397), bc18+a49 (K19398), bc18+a50 (K19399), bc18+a51 (K19400), bc18+a52 (K19401), bc18+a53 (K19402), bc18+a54 (K19403), bc18+a55 (K19404), bc18+a56 (K19405), bc18+a57 (K19406), bc18+a58 (K19407), bc18+a59 (K19408), bc18+a60 (K19409), bc18+a61 (K19410), bc18+a62 (K19411), bc18+a63 (K19412), bc18+a64 (K19413), bc18+a65 (K19414), bc18+a66 (K19415), bc18+a67 (K19416), bc18+a68 (K19417), bc18+a69 (K19418), bc18+a70 (K19419), bc18+a71 (K19420), bc18+a72 (K19421), bc18+a73 (K19422), bc18+a74 (K19423), bc18+a75 (K19424), bc18+a76 (K19425), bc18+a77 (K19426), bc18+a78 (K19427), bc18+a79 (K19428), bc18+a80 (K19429), bc18+a81 (K19430), bc18+a82 (K19431), bc18+a83 (K19432), bc18+a84 (K19433), bc18+a85 (K19434), bc18+a86 (K19435), bc18+a87 (K19436), bc18+a88 (K19437), bc18+a89 (K19438), bc18+a90 (K19439), bc18+a91 (K19440), bc18+a92 (K19441), bc18+a93 (K19442), bc18+a94 (K19443), bc18+a95 (K19444), bc18+a96 (K19445), bc18+a97 (K19446), bc18+a98 (K19447), bc18+a99 (K19448), bc18+a100 (K19449), bc18+a101 (K19450), bc18+a102 (K19451), bc18+a103 (K19452), bc18+a104 (K19453), bc19+a1 (K19454), bc19+a2 (K19455), bc19+a3 (K19456), bc19+a4 (K19457), bc19+a5 (K19458), bc19+a6 (K19459), bc19+a7 (K19460), bc19+a8 (K19461), bc19+a9 (K19462), bc19+a10 (K19463), bc19+a11 (K19464), bc19+a12 (K19465), bc19+a13 (K19466), bc19+a14 (K19467), bc19+a15 (K19468), bc19+a16 (K19469), bc19+a17 (K19470), bc19+a18 (K19471), bc19+a19 (K19472), bc19+a20 (K19473), bc19+a21 (K19474), bc19+a22 (K19475), bc19+a23 (K19476), bc19+a24 (K19477), bc19+a25 (K19478), bc19+a26 (K19479), bc19+a27 (K19480), bc19+a28 (K19481), bc19+a29 (K19482), bc19+a30 (K19483), bc19+a31 (K19484), bc19+a32 (K19485), bc19+a33 (K19486), bc19+a34 (K19487), bc19+a35 (K19488), bc19+a36 (K19489), bc19+a37 (K19490), bc19+a38 (K19491), bc19+a39 (K19492), bc19+a40 (K19493), bc19+a41 (K19494), bc19+a42 (K19495), bc19+a43 (K19496), bc19+a44 (K19497), bc19+a45 (K19498), bc19+a46 (K19499), bc19+a47 (K19500), bc19+a48 (K19501), bc19+a49 (K19502), bc19+a50 (K19503), bc19+a51 (K19504), bc19+a52 (K19505), bc19+a53 (K19506), bc19+a54 (K19507), bc19+a55 (K19508), bc19+a56 (K19509), bc19+a57 (K19510), bc19+a58 (K19511), bc19+a59 (K19512), bc19+a60 (K19513), bc19+a61 (K19514), bc19+a62 (K19515), bc19+a63 (K19516), bc19+a64 (K19517), bc19+a65 (K19518), bc19+a66 (K19519), bc19+a67 (K19520), bc19+a68 (K19521), bc19+a69 (K19522), bc19+a70 (K19523), bc19+a71 (K19524), bc19+a72 (K19525), bc19+a73 (K19526), bc19+a74 (K19527), bc19+a75 (K19528), bc19+a76 (K19529), bc19+a77 (K19530), bc19+a78 (K19531), bc19+a79 (K19532), bc19+a80 (K19533), bc19+a81 (K19534), bc19+a82 (K19535), bc19+a83 (K19536), bc19+a84 (K19537), bc19+a85 (K19538), bc19+a86 (K19539), bc19+a87 (K19540), bc19+a88 (K19541), bc19+a89 (K19542), bc19+a90 (K19543), bc19+a91 (K19544), bc19+a92 (K19545), bc19+a93 (K19546), bc19+a94 (K19547), bc19+a95 (K19548), bc19+a96 (K19549), bc19+a97 (K19550), bc19+a98 (K19551), bc19+a99 (K19552), bc19+a100 (K19553), bc19+a101 (K19554), bc19+a102 (K19555), bc19+a103 (K19556), bc19+a104 (K19557), bc20+a1 (K19558), bc20+a2 (K19559), bc20+a3 (K19560), bc20+a4 (K19561), bc20+a5 (K19562), bc20+a6 (K19563), bc20+a7 (K19564), bc20+a8 (K19565), bc20+a9 (K19566), bc20+a10 (K19567), bc20+a11 (K19568), bc20+a12 (K19569), bc20+a13 (K19570), bc20+a14 (K19571), bc20+a15 (K19572), bc20+a16 (K19573), bc20+a17 (K19574), bc20+a18 (K19575), bc20+a19 (K19576), bc20+a20 (K19577), bc20+a21 (K19578), bc20+a22 (K19579), bc20+a23 (K19580), bc20+a24 (K19581), bc20+a25 (K19582), bc20+a26 (K19583), bc20+a27 (K19584), bc20+a28 (K19585), bc20+a29 (K19586), bc20+a30 (K19587), bc20+a31 (K19588), bc20+a32 (K19589), bc20+a33 (K19590), bc20+a34 (K19591), bc20+a35 (K19592), bc20+a36 (K19593), bc20+a37 (K19594), bc20+a38 (K19595), bc20+a39 (K19596), bc20+a40 (K19597), bc20+a41 (K19598), bc20+a42 (K19599), bc20+a43 (K19600), bc20+a44 (K19601), bc20+a45 (K19602), bc20+a46 (K19603), bc20+a47 (K19604), bc20+a48 (K19605), bc20+a49 (K19606), bc20+a50 (K19607), bc20+a51 (K19608), bc20+a52 (K19609), bc20+a53 (K19610), bc20+a54 (K19611), bc20+a55 (K19612), bc20+a56 (K19613), bc20+a57 (K19614), bc20+a58 (K19615), bc20+a59 (K19616), bc20+a60 (K19617), bc20+a61 (K19618), bc20+a62 (K19619), bc20+a63 (K19620), bc20+a64 (K19621), bc20+a65 (K19622), bc20+a66 (K19623), bc20+a67 (K19624), bc20+a68 (K19625), bc20+a69 (K19626), bc20+a70 (K19627), bc20+a71 (K19628), bc20+a72 (K19629), bc20+a73 (K19630), bc20+a74 (K19631), bc20+a75 (K19632), bc20+a76 (K19633), bc20+a77 (K19634), bc20+a78 (K19635), bc20+a79 (K19636), bc20+a80 (K19637), bc20+a81 (K19638), bc20+a82 (K19639), bc20+a83 (K19640), bc20+a84 (K19641), bc20+a85 (K19642), bc20+a86 (K19643), bc20+a87 (K19644), bc20+a88 (K19645), bc20+a89 (K19646), bc20+a90 (K19647), bc20+a91 (K19648), bc20+a92 (K19649), bc20+a93 (K19650), bc20+a94 (K19651), bc20+a95 (K19652), bc20+a96 (K19653), bc20+a97 (K19654), bc20+a98 (K19655), bc20+a99 (K19656), bc20+a100 (K19657), bc20+a101 (K19658), bc20+a102 (K19659), bc20+a103 (K19660), bc20+a104 (K19661), bc21+a1 (K19662), bc21+a2 (K19663), bc21+a3 (K19664), bc21+a4 (K19665), bc21+a5 (K19666), bc21+a6 (K19667), bc21+a7 (K19668), bc21+a8 (K19669), bc21+a9 (K19670), bc21+a10 (K19671), bc21+a11 (K19672), bc21+a12 (K19673), bc21+a13 (K19674), bc21+a14 (K19675), bc21+a15 (K19676), bc21+a16 (K19677), bc21+a17 (K19678), bc21+a18 (K19679), bc21+a19 (K19680), bc21+a20 (K19681), bc21+a21 (K19682), bc21+a22 (K19683), bc21+a23 (K19684), bc21+a24 (K19685), bc21+a25 (K19686), bc21+a26 (K19687), bc21+a27 (K19688), bc21+a28 (K19689), bc21+a29 (K19690), bc21+a30 (K19691), bc21+a31 (K19692), bc21+a32 (K19693), bc21+a33 (K19694), bc21+a34 (K19695), bc21+a35 (K19696), bc21+a36 (K19697), bc21+a37 (K19698), bc21+a38 (K19699), bc21+a39 (K19700), bc21+a40 (K19701), bc21+a41 (K19702), bc21+a42 (K19703), bc21+a43 (K19704), bc21+a44 (K19705), bc21+a45 (K19706), bc21+a46 (K19707), bc21+a47 (K19708), bc21+a48 (K19709), bc21+a49 (K19710), bc21+a50 (K19711), bc21+a51 (K19712), bc21+a52 (K19713), bc21+a53 (K19714), bc21+a54 (K19715), bc21+a55 (K19716), bc21+a56 (K19717), bc21+a57 (K19718), bc21+a58 (K19719), bc21+a59 (K19720), bc21+a60 (K19721), bc21+a61 (K19722), bc21+a62 (K19723), bc21+a63 (K19724), bc21+a64 (K19725), bc21+a65 (K19726), bc21+a66 (K19727), bc21+a67 (K19728), bc21+a68 (K19729), bc21+a69 (K19730), bc21+a70 (K19731), bc21+a71 (K19732), bc21+a72 (K19733), bc21+a73 (K19734), bc21+a74 (K19735), bc21+a75 (K19736), bc21+a76 (K19737), bc21+a77 (K19738), bc21+a78 (K19739), bc21+a79 (K19740), bc21+a80 (K19741), bc21+a81 (K19742), bc21+a82 (K19743), bc21+a83 (K19744), bc21+a84 (K19745), bc21+a85 (K19746), bc21+a86 (K19747), bc21+a87 (K19748), bc21+a88 (K19749), bc21+a89 (K19750), bc21+a90 (K19751), bc21+a91 (K19752), bc21+a92 (K19753), bc21+a93 (K19754), bc21+a94 (K19755), bc21+a95 (K19756), bc21+a96 (K19757), bc21+a97 (K19758), bc21+a98 (K19759), bc21+a99 (K19760), bc21+a100 (K19761), bc21+a101 (K19762), bc21+a102 (K19763), bc21+a103 (K19764), and bc21+a104 (K19765), and wherein the at least one module (a), the [module (b)+module (c)] protein or peptide, and the at least one compound (d) are arranged in any arrangement and in any stoichiometry. The conjugates of the present invention optionally comprise a nuclear localization signal.

In a preferred embodiment of the second aspect, a conjugate comprised in the delivery system of the present invention comprises, essentially consists of, consists of or contains:

(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
wherein the at least one module (b) and the at least one module (c) are comprised or contained within a [module (b)+module (c)] protein or peptide, and wherein the [module (b)+module (c)] protein or peptide and the at least one module (a) are combined in a combination as indicated by a numerical from K4359 to K4918 or K18142 to K19765, and wherein the combination of the [module (b)+module (c)] protein or peptide and the at least one module (a) is combined with at least one compound (d) according to the following scheme:
KX, in each case combined with at least one compound d1; KX, in each case combined with at least one compound d2; KX, in each case combined with at least one compound d3; KX, in each case combined with at least one compound d4; KX, in each case combined with at least one compound d5; KX, in each case combined with at least one compound d6; KX, in each case combined with at least one compound d7; KX, in each case combined with at least one compound d8; KX, in each case combined with at least one compound d9; KX, in each case combined with at least one compound d10; KX, in each case combined with at least one compound d11; KX, in each case combined with at least one compound d12; KX, in each case combined with at least one compound d13; KX, in each case combined with at least one compound d14; KX, in each case combined with at least one compound d15; KX, in each case combined with at least one compound d16; KX, in each case combined with at least one compound d17; KX, in each case combined with at least one compound d18; KX, in each case combined with at least one compound d19; KX, in each case combined with at least one compound d20; KX, in each case combined with at least one compound d21; KX, in each case combined with at least one compound d22; KX, in each case combined with at least one compound d23; KX, in each case combined with at least one compound d24; KX, in each case combined with at least one compound d25; KX, in each case combined with at least one compound d26; KX, in each case combined with at least one compound d27; KX, in each case combined with at least one compound d28; KX, in each case combined with at least one compound d29; KX, in each case combined with at least one compound d30; KX, in each case combined with at least one compound d31; KX, in each case combined with at least one compound d32; KX, in each case combined with at least one compound d33; KX, in each case combined with at least one compound d34; KX, in each case combined with at least one compound d35; KX, in each case combined with at least one compound d36; KX, in each case combined with at least one compound d37; KX, in each case combined with at least one compound d38; KX, in each case combined with at least one compound d39; KX, in each case combined with at least one compound d40; KX, in each case combined with at least one compound d41; KX, in each case combined with at least one compound d42; KX, in each case combined with at least one compound d43; KX, in each case combined with at least one compound d44; KX, in each case combined with at least one compound d45; KX, in each case combined with at least one compound d46; KX, in each case combined with at least one compound d47; KX, in each case combined with at least one compound d48; KX, in each case combined with at least one compound d49; KX, in each case combined with at least one compound d50; KX, in each case combined with at least one compound d51; KX, in each case combined with at least one compound d52; KX, in each case combined with at least one compound d53; KX, in each case combined with at least one compound d54; KX, in each case combined with at least one compound d55; KX, in each case combined with at least one compound d56; KX, in each case combined with at least one compound d57; KX, in each case combined with at least one compound d58; KX, in each case combined with at least one compound d59; KX, in each case combined with at least one compound d60; KX, in each case combined with at least one compound d61; KX, in each case combined with at least one compound d62; KX, in each case combined with at least one compound d63; KX, in each case combined with at least one compound d64; KX, in each case combined with at least one compound d65; KX, in each case combined with at least one compound d66; KX, in each case combined with at least one compound d67; KX, in each case combined with at least one compound d68; KX, in each case combined with at least one compound d69; KX, in each case combined with at least one compound d70; KX, in each case combined with at least one compound d71; KX, in each case combined with at least one compound d72; KX, in each case combined with at least one compound d73; KX, in each case combined with at least one compound d74; KX, in each case combined with at least one compound d75; KX, in each case combined with at least one compound d76; KX, in each case combined with at least one compound d77; KX, in each case combined with at least one compound d78; KX, in each case combined with at least one compound d79; KX, in each case combined with at least one compound d80; KX, in each case combined with at least one compound d81; KX, in each case combined with at least one compound d82; KX, in each case combined with at least one compound d83; KX, in each case combined with at least one compound d84; KX, in each case combined with at least one compound d85; KX, in each case combined with at least one module d86; KX, in each case combined with at least one compound d87; KX, in each case combined with at least one compound d88; KX, in each case combined with at least one compound d89; KX, in each case combined with at least one compound d90; KX, in each case combined with at least one compound d91; KX, in each case combined with at least one compound d92; KX, in each case combined with at least one compound d93; KX, in each case combined with at least one compound d94; KX, in each case combined with at least one compound d95; KX, in each case combined with at least one module d96; KX, in each case combined with at least one compound d97; KX, in each case combined with at least one compound d98; KX, in each case combined with at least one compound d99; KX, in each case combined with at least one compound d100; KX, in each case combined with at least one compound d101; KX, in each case combined with at least one compound d102; KX, in each case combined with at least one compound d103; KX, in each case combined with at least one compound d104; KX, in each case combined with at least one compound d105; KX, in each case combined with at least one compound d106; KX, in each case combined with at least one compound d107; KX, in each case combined with at least one compound d108; KX, in each case combined with at least one compound d109; KX, in each case combined with at least one compound d110 KX, in each case combined with at least one compound d111; KX, in each case combined with at least one compound d112; KX, in each case combined with at least one compound d113; KX, in each case combined with at least one compound d114; KX, in each case combined with at least one compound d115; KX, in each case combined with at least one compound d116; KX, in each case combined with at least one compound d117; KX, in each case combined with at least one compound d118; KX, in each case combined with at least one compound d119; KX, in each case combined with at least one compound d120; KX, in each case combined with at least one compound d121; KX, in each case combined with at least one compound d122; KX, in each case combined with at least one compound d123; KX, in each case combined with at least one compound d124; KX, in each case combined with at least one compound d125; KX, in each case combined with at least one compound d126; KX, in each case combined with at least one compound d127; KX, in each case combined with at least one compound d128; KX, in each case combined with at least one compound d129; KX, in each case combined with at least one compound d130; KX, in each case combined with at least one compound d131; KX, in each case combined with at least one compound d132; KX, in each case combined with at least one compound d133; KX, in each case combined with at least one compound d134; KX, in each case combined with at least one compound d135; KX, in each case combined with at least one compound d136; KX, in each case combined with at least one compound d137; KX, in each case combined with at least one compound d138; KX, in each case combined with at least one compound d139; KX, in each case combined with at least one compound d140; KX, in each case combined with at least one compound d141; KX, in each case combined with at least one compound d142; KX, in each case combined with at least one compound d143; KX, in each case combined with at least one compound d144; KX, in each case combined with at least one compound d145; KX, in each case combined with at least one compound d146; KX, in each case combined with at least one compound d147; KX, in each case combined with at least one compound d148; KX, in each case combined with at least one compound d149; KX, in each case combined with at least one compound d150; KX, in each case combined with at least one compound d151; KX, in each case combined with at least one compound d152; KX, in each case combined with at least one compound d153; KX, in each case combined with at least one compound d154; KX, in each case combined with at least one compound d155; KX, in each case combined with at least one compound d156; KX, in each case combined with at least one compound d157; KX, in each case combined with at least one compound d158; KX, in each case combined with at least one compound d159; KX, in each case combined with at least one compound d160; KX, in each case combined with at least one compound d161; KX, in each case combined with at least one compound d162; KX, in each case combined with at least one compound d163; KX, in each case combined with at least one compound d164; KX, in each case combined with at least one compound d165; KX, in each case combined with at least one compound d166; KX, in each case combined with at least one compound d167; KX, in each case combined with at least one compound d168; KX, in each case combined with at least one compound d169; KX, in each case combined with at least one compound d170;
wherein X is the combination of the [module (b)+module (c)] protein or peptide and the at least one module (a) and has the following meaning: 4359, 4360, 4361, 4362, 4363, 4364, 4365, 4366, 4367, 4368, 4369, 4370, 4371, 4372, 4373, 4374, 4375, 4376, 4377, 4378, 4379, 4380, 4381, 4382, 4383, 4384, 4385, 4386, 4387, 4388, 4389, 4390, 4391, 4392, 4393, 4394, 4395, 4396, 4397, 4398, 4399, 4400, 4401, 4402, 4403, 4404, 4405, 4406, 4407, 4408, 4409, 4410, 4411, 4412, 4413, 4414, 4415, 4416, 4417, 4418, 4419, 4420, 4421, 4422, 4423, 4424, 4425, 4426, 4427, 4428, 4429, 4430, 4431, 4432, 4433, 4434, 4435, 4436, 4437, 4438, 4439, 4440, 4441, 4442, 4443, 4444, 4445, 4446, 4447, 4448, 4449, 4450, 4451, 4452, 4453, 4454, 4455, 4456, 4457, 4458, 4459, 4460, 4461, 4462, 4463, 4464, 4465, 4466, 4467, 4468, 4469, 4470, 4471, 4472, 4473, 4474, 4475, 4476, 4477, 4478, 4479, 4480, 4481, 4482, 4483, 4484, 4485, 4486, 4487, 4488, 4489, 4490, 4491, 4492, 4493, 4494, 4495, 4496, 4497, 4498, 4499, 4500, 4501, 4502, 4503, 4504, 4505, 4506, 4507, 4508, 4509, 4510, 4511, 4512, 4513, 4514, 4515, 4516, 4517, 4518, 4519, 4520, 4521, 4522, 4523, 4524, 4525, 4526, 4527, 4528, 4529, 4530, 4531, 4532, 4533, 4534, 4535, 4536, 4537, 4538, 4539, 4540, 4541, 4542, 4543, 4544, 4545, 4546, 4547, 4548, 4549, 4550, 4551, 4552, 4553, 4554, 4555, 4556, 4557, 4558, 4559, 4560, 4561, 4562, 4563, 4564, 4565, 4566, 4567, 4568, 4569, 4570, 4571, 4572, 4573, 4574, 4575, 4576, 4577, 4578, 4579, 4580, 4581, 4582, 4583, 4584, 4585, 4586, 4587, 4588, 4589, 4590, 4591, 4592, 4593, 4594, 4595, 4596, 4597, 4598, 4599, 4600, 4601, 4602, 4603, 4604, 4605, 4606, 4607, 4608, 4609, 4610, 4611, 4612, 4613, 4614, 4615, 4616, 4617, 4618, 4619, 4620, 4621, 4622, 4623, 4624, 4625, 4626, 4627, 4628, 4629, 4630, 4631, 4632, 4633, 4634, 4635, 4636, 4637, 4638, 4639, 4640, 4641, 4642, 4643, 4644, 4645, 4646, 4647, 4648, 4649, 4650, 4651, 4652, 4653, 4654, 4655, 4656, 4657, 4658, 4659, 4660, 4661, 4662, 4663, 4664, 4665, 4666, 4667, 4668, 4669, 4670, 4671, 4672, 4673, 4674, 4675, 4676, 4677, 4678, 4679, 4680, 4681, 4682, 4683, 4684, 4685, 4686, 4687, 4688, 4689, 4690, 4691, 4692, 4693, 4694, 4695, 4696, 4697, 4698, 4699, 4700, 4701, 4702, 4703, 4704, 4705, 4706, 4707, 4708, 4709, 4710, 4711, 4712, 4713, 4714, 4715, 4716, 4717, 4718, 4719, 4720, 4721, 4722, 4723, 4724, 4725, 4726, 4727, 4728, 4729, 4730, 4731, 4732, 4733, 4734, 4735, 4736, 4737, 4738, 4739, 4740, 4741, 4742, 4743, 4744, 4745, 4746, 4747, 4748, 4749, 4750, 4751, 4752, 4753, 4754, 4755, 4756, 4757, 4758, 4759, 4760, 4761, 4762, 4763, 4764, 4765, 4766, 4767, 4768, 4769, 4770, 4771, 4772, 4773, 4774, 4775, 4776, 4777, 4778, 4779, 4780, 4781, 4782, 4783, 4784, 4785, 4786, 4787, 4788, 4789, 4790, 4791, 4792, 4793, 4794, 4795, 4796, 4797, 4798, 4799, 4800, 4801, 4802, 4803, 4804, 4805, 4806, 4807, 4808, 4809, 4810, 4811, 4812, 4813, 4814, 4815, 4816, 4817, 4818, 4819, 4820, 4821, 4822, 4823, 4824, 4825, 4826, 4827, 4828, 4829, 4830, 4831, 4832, 4833, 4834, 4835, 4836, 4837, 4838, 4839, 4840, 4841, 4842, 4843, 4844, 4845, 4846, 4847, 4848, 4849, 4850, 4851, 4852, 4853, 4854, 4855, 4856, 4857, 4858, 4859, 4860, 4861, 4862, 4863, 4864, 4865, 4866, 4867, 4868, 4869, 4870, 4871, 4872, 4873, 4874, 4875, 4876, 4877, 4878, 4879, 4880, 4881, 4882, 4883, 4884, 4885, 4886, 4887, 4888, 4889, 4890, 4891, 4892, 4893, 4894, 4895, 4896, 4897, 4898, 4899, 4900, 4901, 4902, 4903, 4904, 4905, 4906, 4907, 4908, 4909, 4910, 4911, 4912, 4913, 4914, 4915, 4916, 4917, 4918, 18142, 18143, 18144, 18145, 18146, 18147, 18148, 18149, 18150, 18151, 18152, 18153, 18154, 18155, 18156, 18157, 18158, 18159, 18160, 18161, 18162, 18163, 18164, 18165, 18166, 18167, 18168, 18169, 18170, 18171, 18172, 18173, 18174, 18175, 18176, 18177, 18178, 18179, 18180, 18181, 18182, 18183, 18184, 18185, 18186, 18187, 18188, 18189, 18190, 18191, 18192, 18193, 18194, 18195, 18196, 18197, 18198, 18199, 18200, 18201, 18202, 18203, 18204, 18205, 18206, 18207, 18208, 18209, 18210, 18211, 18212, 18213, 18214, 18215, 18216, 18217, 18218, 18219, 18220, 18221, 18222, 18223, 18224, 18225, 18226, 18227, 18228, 18229, 18230, 18231, 18232, 18233, 18234, 18235, 18236, 18237, 18238, 18239, 18240, 18241, 18242, 18243, 18244, 18245, 18246, 18247, 18248, 18249, 18250, 18251, 18252, 18253, 18254, 18255, 18256, 18257, 18258, 18259, 18260, 18261, 18262, 18263, 18264, 18265, 18266, 18267, 18268, 18269, 18270, 18271, 18272, 18273, 18274, 18275, 18276, 18277, 18278, 18279, 18280, 18281, 18282, 18283, 18284, 18285, 18286, 18287, 18288, 18289, 18290, 18291, 18292, 18293, 18294, 18295, 18296, 18297, 18298, 18299, 18300, 18301, 18302, 18303, 18304, 18305, 18306, 18307, 18308, 18309, 18310, 18311, 18312, 18313, 18314, 18315, 18316, 18317, 18318, 18319, 18320, 18321, 18322, 18323, 18324, 18325, 18326, 18327, 18328, 18329, 18330, 18331, 18332, 18333, 18334, 18335, 18336, 18337, 18338, 18339, 18340, 18341, 18342, 18343, 18344, 18345, 18346, 18347, 18348, 18349, 18350, 18351, 18352, 18353, 18354, 18355, 18356, 18357, 18358, 18359, 18360, 18361, 18362, 18363, 18364, 18365, 18366, 18367, 18368, 18369, 18370, 18371, 18372, 18373, 18374, 18375, 18376, 18377, 18378, 18379, 18380, 18381, 18382, 18383, 18384, 18385, 18386, 18387, 18388, 18389, 18390, 18391, 18392, 18393, 18394, 18395, 18396, 18397, 18398, 18399, 18400, 18401, 18402, 18403, 18404, 18405, 18406, 18407, 18408, 18409, 18410, 18411, 18412, 18413, 18414, 18415, 18416, 18417, 18418, 18419, 18420, 18421, 18422, 18423, 18424, 18425, 18426, 18427, 18428, 18429, 18430, 18431, 18432, 18433, 18434, 18435, 18436, 18437, 18438, 18439, 18440, 18441, 18442, 18443, 18444, 18445, 18446, 18447, 18448, 18449, 18450, 18451, 18452, 18453, 18454, 18455, 18456, 18457, 18458, 18459, 18460, 18461, 18462, 18463, 18464, 18465, 18466, 18467, 18468, 18469, 18470, 18471, 18472, 18473, 18474, 18475, 18476, 18477, 18478, 18479, 18480, 18481, 18482, 18483, 18484, 18485, 18486, 18487, 18488, 18489, 18490, 18491, 18492, 18493, 18494, 18495, 18496, 18497, 18498, 18499, 18500, 18501, 18502, 18503, 18504, 18505, 18506, 18507, 18508, 18509, 18510, 18511, 18512, 18513, 18514, 18515, 18516, 18517, 18518, 18519, 18520, 18521, 18522, 18523, 18524, 18525, 18526, 18527, 18528, 18529, 18530, 18531, 18532, 18533, 18534, 18535, 18536, 18537, 18538, 18539, 18540, 18541, 18542, 18543, 18544, 18545, 18546, 18547, 18548, 18549, 18550, 18551, 18552, 18553, 18554, 18555, 18556, 18557, 18558, 18559, 18560, 18561, 18562, 18563, 18564, 18565, 18566, 18567, 18568, 18569, 18570, 18571, 18572, 18573, 18574, 18575, 18576, 18577, 18578, 18579, 18580, 18581, 18582, 18583, 18584, 18585, 18586, 18587, 18588, 18589, 18590, 18591, 18592, 18593, 18594, 18595, 18596, 18597, 18598, 18599, 18600, 18601, 18602, 18603, 18604, 18605, 18606, 18607, 18608, 18609, 18610, 18611, 18612, 18613, 18614, 18615, 18616, 18617, 18618, 18619, 18620, 18621, 18622, 18623, 18624, 18625, 18626, 18627, 18628, 18629, 18630, 18631, 18632, 18633, 18634, 18635, 18636, 18637, 18638, 18639, 18640, 18641, 18642, 18643, 18644, 18645, 18646, 18647, 18648, 18649, 18650, 18651, 18652, 18653, 18654, 18655, 18656, 18657, 18658, 18659, 18660, 18661, 18662, 18663, 18664, 18665, 18666, 18667, 18668, 18669, 18670, 18671, 18672, 18673, 18674, 18675, 18676, 18677, 18678, 18679, 18680, 18681, 18682, 18683, 18684, 18685, 18686, 18687, 18688, 18689, 18690, 18691, 18692, 18693, 18694, 18695, 18696, 18697, 18698, 18699, 18700, 18701, 18702, 18703, 18704, 18705, 18706, 18707, 18708, 18709, 18710, 18711, 18712, 18713, 18714, 18715, 18716, 18717, 18718, 18719, 18720, 18721, 18722, 18723, 18724, 18725, 18726, 18727, 18728, 18729, 18730, 18731, 18732, 18733, 18734, 18735, 18736, 18737, 18738, 18739, 18740, 18741, 18742, 18743, 18744, 18745, 18746, 18747, 18748, 18749, 18750, 18751, 18752, 18753, 18754, 18755, 18756, 18757, 18758, 18759, 18760, 18761, 18762, 18763, 18764, 18765, 18766, 18767, 18768, 18769, 18770, 18771, 18772, 18773, 18774, 18775, 18776, 18777, 18778, 18779, 18780, 18781, 18782, 18783, 18784, 18785, 18786, 18787, 18788, 18789, 18790, 18791, 18792, 18793, 18794, 18795, 18796, 18797, 18798, 18799, 18800, 18801, 18802, 18803, 18804, 18805, 18806, 18807, 18808, 18809, 18810, 18811, 18812, 18813, 18814, 18815, 18816, 18817, 18818, 18819, 18820, 18821, 18822, 18823, 18824, 18825, 18826, 18827, 18828, 18829, 18830, 18831, 18832, 18833, 18834, 18835, 18836, 18837, 18838, 18839, 18840, 18841, 18842, 18843, 18844, 18845, 18846, 18847, 18848, 18849, 18850, 18851, 18852, 18853, 18854, 18855, 18856, 18857, 18858, 18859, 18860, 18861, 18862, 18863, 18864, 18865, 18866, 18867, 18868, 18869, 18870, 18871, 18872, 18873, 18874, 18875, 18876, 18877, 18878, 18879, 18880, 18881, 18882, 18883, 18884, 18885, 18886, 18887, 18888, 18889, 18890, 18891, 18892, 18893, 18894, 18895, 18896, 18897, 18898, 18899, 18900, 18901, 18902, 18903, 18904, 18905, 18906, 18907, 18908, 18909, 18910, 18911, 18912, 18913, 18914, 18915, 18916, 18917, 18918, 18919, 18920, 18921, 18922, 18923, 18924, 18925, 18926, 18927, 18928, 18929, 18930, 18931, 18932, 18933, 18934, 18935, 18936, 18937, 18938, 18939, 18940, 18941, 18942, 18943, 18944, 18945, 18946, 18947, 18948, 18949, 18950, 18951, 18952, 18953, 18954, 18955, 18956, 18957, 18958, 18959, 18960, 18961, 18962, 18963, 18964, 18965, 18966, 18967, 18968, 18969, 18970, 18971, 18972, 18973, 18974, 18975, 18976, 18977, 18978, 18979, 18980, 18981, 18982, 18983, 18984, 18985, 18986, 18987, 18988, 18989, 18990, 18991, 18992, 18993, 18994, 18995, 18996, 18997, 18998, 18999, 19000, 19001, 19002, 19003, 19004, 19005, 19006, 19007, 19008, 19009, 19010, 19011, 19012, 19013, 19014, 19015, 19016, 19017, 19018, 19019, 19020, 19021, 19022, 19023, 19024, 19025, 19026, 19027, 19028, 19029, 19030, 19031, 19032, 19033, 19034, 19035, 19036, 19037, 19038, 19039, 19040, 19041, 19042, 19043, 19044, 19045, 19046, 19047, 19048, 19049, 19050, 19051, 19052, 19053, 19054, 19055, 19056, 19057, 19058, 19059, 19060, 19061, 19062, 19063, 19064, 19065, 19066, 19067, 19068, 19069, 19070, 19071, 19072, 19073, 19074, 19075, 19076, 19077, 19078, 19079, 19080, 19081, 19082, 19083, 19084, 19085, 19086, 19087, 19088, 19089, 19090, 19091, 19092, 19093, 19094, 19095, 19096, 19097, 19098, 19099, 19100, 19101, 19102, 19103, 19104, 19105, 19106, 19107, 19108, 19109, 19110, 19111, 19112, 19113, 19114, 19115, 19116, 19117, 19118, 19119, 19120, 19121, 19122, 19123, 19124, 19125, 19126, 19127, 19128, 19129, 19130, 19131, 19132, 19133, 19134, 19135, 19136, 19137, 19138, 19139, 19140, 19141, 19142, 19143, 19144, 19145, 19146, 19147, 19148, 19149, 19150, 19151, 19152, 19153, 19154, 19155, 19156, 19157, 19158, 19159, 19160, 19161, 19162, 19163, 19164, 19165, 19166, 19167, 19168, 19169, 19170, 19171, 19172, 19173, 19174, 19175, 19176, 19177, 19178, 19179, 19180, 19181, 19182, 19183, 19184, 19185, 19186, 19187, 19188, 19189, 19190, 19191, 19192, 19193, 19194, 19195, 19196, 19197, 19198, 19199, 19200, 19201, 19202, 19203, 19204, 19205, 19206, 19207, 19208, 19209, 19210, 19211, 19212, 19213, 19214, 19215, 19216, 19217, 19218, 19219, 19220, 19221, 19222, 19223, 19224, 19225, 19226, 19227, 19228, 19229, 19230, 19231, 19232, 19233, 19234, 19235, 19236, 19237, 19238, 19239, 19240, 19241, 19242, 19243, 19244, 19245, 19246, 19247, 19248, 19249, 19250, 19251, 19252, 19253, 19254, 19255, 19256, 19257, 19258, 19259, 19260, 19261, 19262, 19263, 19264, 19265, 19266, 19267, 19268, 19269, 19270, 19271, 19272, 19273, 19274, 19275, 19276, 19277, 19278, 19279, 19280, 19281, 19282, 19283, 19284, 19285, 19286, 19287, 19288, 19289, 19290, 19291, 19292, 19293, 19294, 19295, 19296, 19297, 19298, 19299, 19300, 19301, 19302, 19303, 19304, 19305, 19306, 19307, 19308, 19309, 19310, 19311, 19312, 19313, 19314, 19315, 19316, 19317, 19318, 19319, 19320, 19321, 19322, 19323, 19324, 19325, 19326, 19327, 19328, 19329, 19330, 19331, 19332, 19333, 19334, 19335, 19336, 19337, 19338, 19339, 19340, 19341, 19342, 19343, 19344, 19345, 19346, 19347, 19348, 19349, 19350, 19351, 19352, 19353, 19354, 19355, 19356, 19357, 19358, 19359, 19360, 19361, 19362, 19363, 19364, 19365, 19366, 19367, 19368, 19369, 19370, 19371, 19372, 19373, 19374, 19375, 19376, 19377, 19378, 19379, 19380, 19381, 19382, 19383, 19384, 19385, 19386, 19387, 19388, 19389, 19390, 19391, 19392, 19393, 19394, 19395, 19396, 19397, 19398, 19399, 19400, 19401, 19402, 19403, 19404, 19405, 19406, 19407, 19408, 19409, 19410, 19411, 19412, 19413, 19414, 19415, 19416, 19417, 19418, 19419, 19420, 19421, 19422, 19423, 19424, 19425, 19426, 19427, 19428, 19429, 19430, 19431, 19432, 19433, 19434, 19435, 19436, 19437, 19438, 19439, 19440, 19441, 19442, 19443, 19444, 19445, 19446, 19447, 19448, 19449, 19450, 19451, 19452, 19453, 19454, 19455, 19456, 19457, 19458, 19459, 19460, 19461, 19462, 19463, 19464, 19465, 19466, 19467, 19468, 19469, 19470, 19471, 19472, 19473, 19474, 19475, 19476, 19477, 19478, 19479, 19480, 19481, 19482, 19483, 19484, 19485, 19486, 19487, 19488, 19489, 19490, 19491, 19492, 19493, 19494, 19495, 19496, 19497, 19498, 19499, 19500, 19501, 19502, 19503, 19504, 19505, 19506, 19507, 19508, 19509, 19510, 19511, 19512, 19513, 19514, 19515, 19516, 19517, 19518, 19519, 19520, 19521, 19522, 19523, 19524, 19525, 19526, 19527, 19528, 19529, 19530, 19531, 19532, 19533, 19534, 19535, 19536, 19537, 19538, 19539, 19540, 19541, 19542, 19543, 19544, 19545, 19546, 19547, 19548, 19549, 19550, 19551, 19552, 19553, 19554, 19555, 19556, 19557, 19558, 19559, 19560, 19561, 19562, 19563, 19564, 19565, 19566, 19567, 19568, 19569, 19570, 19571, 19572, 19573, 19574, 19575, 19576, 19577, 19578, 19579, 19580, 19581, 19582, 19583, 19584, 19585, 19586, 19587, 19588, 19589, 19590, 19591, 19592, 19593, 19594, 19595, 19596, 19597, 19598, 19599, 19600, 19601, 19602, 19603, 19604, 19605, 19606, 19607, 19608, 19609, 19610, 19611, 19612, 19613, 19614, 19615, 19616, 19617, 19618, 19619, 19620, 19621, 19622, 19623, 19624, 19625, 19626, 19627, 19628, 19629, 19630, 19631, 19632, 19633, 19634, 19635, 19636, 19637, 19638, 19639, 19640, 19641, 19642, 19643, 19644, 19645, 19646, 19647, 19648, 19649, 19650, 19651, 19652, 19653, 19654, 19655, 19656, 19657, 19658, 19659, 19660, 19661, 19662, 19663, 19664, 19665, 19666, 19667, 19668, 19669, 19670, 19671, 19672, 19673, 19674, 19675, 19676, 19677, 19678, 19679, 19680, 19681, 19682, 19683, 19684, 19685, 19686, 19687, 19688, 19689, 19690, 19691, 19692, 19693, 19694, 19695, 19696, 19697, 19698, 19699, 19700, 19701, 19702, 19703, 19704, 19705, 19706, 19707, 19708, 19709, 19710, 19711, 19712, 19713, 19714, 19715, 19716, 19717, 19718, 19719, 19720, 19721, 19722, 19723, 19724, 19725, 19726, 19727, 19728, 19729, 19730, 19731, 19732, 19733, 19734, 19735, 19736, 19737, 19738, 19739, 19740, 19741, 19742, 19743, 19744, 19745, 19746, 19747, 19748, 19749, 19750, 19751, 19752, 19753, 19754, 19755, 19756, 19757, 19758, 19759, 19760, 19761, 19762, 19763, 19764, or 19765, and wherein the [module (b)+module (c)] protein or peptide, the at least one module (a), and the at least one compound (d) are linked to each other in any arrangement and stoichiometry.

In a preferred embodiment of the second aspect, the present invention relates to a delivery system for delivery of a compound into a cell comprising or consisting of at least one conjugate comprising, essentially consisting of or consisting of:

(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
wherein the at least one module (a) and the at least one module (c) are comprised or contained within a [module (a)+module (c)] protein or peptide, and wherein the a [module (a)+module (c)] protein or peptide, the at least one module (b), and the at least one compound (d) are linked to each other in any arrangement. The conjugates of the present invention optionally comprise a nuclear localization signal.

In a preferred embodiment of the second aspect, the present invention relates to a delivery system for delivery of a compound into a cell comprising or consisting of at least one conjugate comprising, essentially consisting of, consisting of or containing:

(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
wherein the at least one module (a), the at least one module (b), and the at least one module (c) are comprised or contained within a [module (a)+module (b)+module (c)] protein or peptide, and wherein the [module (a)+module (b)+module (c)] protein or peptide and the at least one compound (d) are linked to each other in any arrangement or stoichiometry. The conjugates of the present invention optionally comprise a nuclear localization signal.

Preferably, within the conjugates, the [module (a)+module (b)+module (c)] protein or peptide comprises, consists essentially of, consists of or contains a mutated a holo-toxin having reduced or no toxicity (abc1), a toxin protein comprising a subunit having reduced or toxicity (abc2), a toxin protein comprising an A-subunit having reduced or no toxicity (abc3), a ricin holo-toxin having reduced or no toxicity (abc4), a ricin toxin protein comprising a subunit having reduced or no toxicity (abc5), a ricin toxin protein comprising an A-subunit having reduced or no toxicity (abc6), a ricin toxin protein comprising an A-subunit that comprises an R180H mutation (SEQ ID NO: 1) (abc7), a ricin holo-toxin comprising an A-subunit having reduced or no toxicity (abc8), a cholera holo-toxin having reduced or no toxicity (abc9), a cholera toxin protein comprising a subunit having reduced or no toxicity (abc10), a cholera toxin protein comprising an A-subunit having reduced or no toxicity (abc11), a Shiga holo-toxin having reduced or no toxicity (abc12), a Shiga toxin protein comprising a subunit having reduced or no toxicity (abc13), a Shiga toxin protein comprising an A-subunit having reduced or no toxicity (abc14), an Stx1a Shiga holo-toxin having reduced or no toxicity (abc15), an Stx1a Shiga toxin protein comprising a subunit having reduced or no toxicity (abc16), an Stx1a Shiga toxin protein comprising an A-subunit having reduced or no toxicity (abc17), an LT holo-toxin having reduced or no toxicity (abc18), an LT toxin protein comprising a subunit having reduced or no toxicity (abc19), an LT toxin protein comprising an A-subunit having reduced or no toxicity (abc20), a pertussis holo-toxin having reduced or no toxicity (abc21), a pertussis toxin protein comprising a subunit having reduced or no toxicity (abc22), a pertussis toxin protein comprising an A-subunit having reduced or no toxicity (abc23), a Pseudomonas exotoxin A holo-toxin having reduced or no toxicity (abc24), a Pseudomonas exotoxin A protein having reduced or no toxicity (abc25), a hybrid toxin having reduced or no toxicity and comprising a mutated A-subunit of a first AB toxin and a B-subunit of a second and different AB toxin (abc26), a hybrid toxin having reduced or no toxicity and comprising a mutated A1-subunit of a first AB₅ toxin and a B-subunit of a second and different AB₅ toxin (abc27), a hybrid ricin-abrin toxin having reduced or no toxicity (abc28), a hybrid ricin-modeccin toxin having reduced or no toxicity (abc29), a hybrid ricin-viscumin toxin having reduced or no toxicity (abc30), a hybrid ricin-volkensin toxin having reduced or no toxicity (abc31), a hybrid abrin-modeccin toxin having reduced or no toxicity (abc32), a hybrid abrin-viscumin toxin having reduced or no toxicity (abc33), a hybrid abrin-volkensin toxin having reduced or no toxicity (abc34), a hybrid modeccin-viscumin toxin having reduced or no toxicity (abc35), a hybrid modeccin-volkensin toxin having reduced or no toxicity (abc36), a hybrid viscumin-volkensin toxin having reduced or no toxicity (abc37), a hybrid LT-cholera toxin having reduced or no toxicity (abc38), a hybrid cholera-Shiga toxin having reduced or no toxicity (abc39), a hybrid cholera-pertussis toxin having reduced or no toxicity (abc40), a hybrid Shiga-Shiga toxin having reduced or no toxicity (abc41), a hybrid Shiga-LT toxin having reduced or no toxicity (abc42), a hybrid Shiga-pertussis toxin having reduced or no toxicity (abc43), a hybrid LT-pertussis toxin having reduced or no toxicity (abc44), a hybrid A1(LT1)-A2(CT)-B5(CT) toxin protein having reduced or no toxicity (abc45), a hybrid A1(ST)-A2(ST)-B5(ST) toxin protein having reduced or no toxicity (abc46), an Stx1b (VT1b) Shiga holo-toxin having reduced or no toxicity (abc47), an Stx1b (VT1b) Shiga toxin protein comprising a subunit having reduced or no toxicity (abc48), an Stx1b (VT1b) Shiga toxin protein comprising an A-subunit having reduced or no toxicity (abc49), an Stx1c (VT1c) Shiga holo-toxin having reduced or no toxicity (abc50), an Stx1c (VT1c) Shiga toxin protein comprising a subunit having reduced or no toxicity (abc51), an Stx1c (VT1c) Shiga toxin protein comprising an A-subunit having reduced or no toxicity (abc52), an Stx1d (VT1d) Shiga holo-toxin having reduced or no toxicity (abc53), an Stx1d (VT1d) Shiga toxin protein comprising a subunit having reduced or no toxicity (abc54), an Stx1d (VT1d) Shiga toxin protein comprising an A-subunit having reduced or no toxicity (abc55), an Stx2a (VT2a) Shiga holo-toxin having reduced or no toxicity (abc56), an Stx2a (VT2a) Shiga toxin protein comprising a subunit having reduced or no toxicity (abc57), an Stx2a (VT2a) Shiga toxin protein comprising an A-subunit having reduced or no toxicity (abc58), an Stx2b (VT2b) Shiga holo-toxin having reduced or no toxicity (abc59), an Stx2b (VT2b) Shiga toxin protein comprising a subunit having reduced or no toxicity (abc60), an Stx2b (VT2b) Shiga toxin protein comprising an A-subunit having reduced or no toxicity (abc61), an Stx2c (VT2c) Shiga holo-toxin having reduced or no toxicity (abc62), an Stx2c (VT2c) Shiga toxin protein comprising a subunit having reduced or no toxicity (abc63), an Stx2c (VT2c) Shiga toxin protein comprising an A-subunit having reduced or no toxicity (abc64), an Stx2d (VT2d) Shiga holo-toxin having reduced or no toxicity (abc65), an Stx2d (VT2d) Shiga toxin protein comprising a subunit having reduced or no toxicity (abc66), an Stx2d (VT2d) Shiga toxin protein comprising an A-subunit having reduced or no toxicity (abc67), an Stx2e (VT2e) Shiga holo-toxin having reduced or no toxicity (abc68), an Stx2e (VT2e) Shiga toxin protein comprising a subunit having reduced or no toxicity (abc69), an Stx2e (VT2e) Shiga toxin protein comprising an A-subunit having reduced or no toxicity (abc70), an Stx2f (VT2f) Shiga holo-toxin having reduced or no toxicity (abc71), an Stx2f (VT2f) Shiga toxin protein comprising a subunit having reduced or no toxicity (abc72), an Stx2f (VT2f) Shiga toxin protein comprising an A-subunit having reduced or no toxicity (abc73), an Stx2g (VT2g) Shiga holo-toxin having reduced or no toxicity (abc74), an Stx2g (VT2g) Shiga toxin protein comprising a subunit having reduced or no toxicity (abc75), or an Stx2g (VT2g) Shiga toxin protein comprising an A-subunit having reduced or no toxicity (abc76). The conjugates of the present invention optionally comprise a nuclear localization signal.

Preferably when the [module (a)+module (b)+module (c)] protein or peptide is a hybrid Shiga-Shiga toxin having reduced or no toxicity (abc41), the hybrid Shiga-Shiga toxin having reduced or no toxicity is a hybrid of two different Shiga toxins selected from the group consisting of Stx1a, Stx1b (VT1b), Stx1c (VT1c), Stx1d (VT1d), Stx2a (VT2a), Stx2b (VT2b), Stx2c (VT2c), Stx2d (VT2d), Stx2e (VT2e), Stx2f (VT2f) and Stx2g (VT2g).

Preferably, the [module (a)+module (b)+module (c)] protein or peptide of the conjugate of the present invention comprises, consists essentially of, or consists of a hybrid toxin having reduced or no toxicity comprising a mutated A-subunit or mutated A1-subunit of a ricin, an abrin a, an abrin b, an abrin c, an abrin d, a modeccin, a viscumin, a volkensin, a cholera toxin, a Shiga toxin, an Stx1a Shiga toxin, an Stx1b (VT1b) Shiga toxin, an Stx1c (VT1c) Shiga toxin, an Stx1d (VT1d) Shiga toxin, an Stx2a (VT2a) Shiga toxin, an Stx2b (VT12b) Shiga toxin, an Stx2c (VT2c) Shiga toxin, an Stx2d (VT2d) Shiga toxin, an Stx2e (VT2e) Shiga toxin, an Stx2f (VT2f) Shiga toxin, an Stx2g (VT2g) Shiga toxin, an E. coli heat-labile enterotoxin, or a pertussis toxin.

Thus, a preferred conjugate comprises, consists essentially of, consists of or contains:

(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
wherein the at least one module (a), the at least one module (b), and the at least one module (c) are comprised or contained within a [module (a)+module (b)+module (c)] protein or peptide, wherein the [module (a)+module (b)+module (c)] protein or peptide comprises, consists essentially of, consists of or contains abc1, abc2, abc3, abc4, abc5, abc6, abc7, abc8, abc9, abc10, abc11, abc12, abc13, abc14, abc15, abc16, abc17, abc18, abc19, abc20, abc21, abc22, abc23, abc24, abc25, abc26, abc27, abc28, abc29, abc30, abc31, abc32, abc33, abc34, abc35, abc36, abc37, abc38, abc39, abc40, abc41, abc42, abc43, abc44, abc45, abc46, abc47, abc48,abc49, abc50, abc51, abc52, abc53, abc54, abc55, abc56, abc57, abc58, abc59, abc60, abc61, abc62, abc63, abc64, abc65, abc66, abc67, abc68, abc69, abc70, abc71, abc72, abc73, abc74, abc75, or abc76, and wherein the [module (a)+module (b)+module (c)] protein or peptide and the at least one compound (d) are linked to each other in any arrangement or stoichiometry. The conjugates of the present invention optionally comprise a nuclear localization signal.

Preferably, a conjugate comprises, essentially consists of, consists of or contains:

(a) at least one module (a) that mediates cell targeting and facilitates cellular uptake,
(b) at least one module (b) that facilitates transport to the ER,
(c) at least one module (c) that mediates translocation from the ER to the cytosol, and
(d) at least one compound (d),
wherein the at least one module (a), the at least one module (b), and the at least one module (c) are comprised or contained within a [module (a)+module (b)+module (c)] protein or peptide, and wherein the [module (a)+module (b)+module (c)] protein or peptide is abc1, abc2, abc3, abc4, abc5, abc6, abc7, abc8, abc9, abc10, abc11, abc12, abc13, abc14, abc15, abc16, abc17, abc18, abc19, abc20, abc21, abc22, abc23, abc24, abc25, abc26, abc27, abc28, abc29, abc30, abc31, abc32, abc33, abc34, abc35, abc36, abc37, abc38, abc39, abc40, abc41, abc42, abc43, abc44, abc45, abc46, abc47, abc48, abc49, abc50, abc51, abc52, abc53, abc54, abc55, abc56, abc57, abc58, abc59, abc60, abc61, abc62, abc63, abc64, abc65, abc66, abc67, abc68, abc69, abc70, abc71, abc72, abc73, abc74, abc75, or abc76, and is combined with the at least one compound (d) according to the following scheme:
X, in each case combined with at least one compound d1; X, in each case combined with at least one compound d2; X, in each case combined with at least one compound d3; X, in each case combined with at least one compound d4; X, in each case combined with at least one compound d5; X, in each case combined with at least one compound d6; X, in each case combined with at least one compound d7; X, in each case combined with at least one compound d8; X, in each case combined with at least one compound d9; X, in each case combined with at least one compound d10; X, in each case combined with at least one compound d11; X, in each case combined with at least one compound d12; X, in each case combined with at least one compound d13; X, in each case combined with at least one compound d14; X, in each case combined with at least one compound d15; X, in each case combined with at least one compound d16; X, in each case combined with at least one compound d17; X, in each case combined with at least one compound d18; X, in each case combined with at least one compound d19; X, in each case combined with at least one compound d20; X, in each case combined with at least one compound d21; X, in each case combined with at least one compound d22; X, in each case combined with at least one compound d23; X, in each case combined with at least one compound d24; X, in each case combined with at least one compound d25; X, in each case combined with at least one compound d26; X, in each case combined with at least one compound d27; X, in each case combined with at least one compound d28; X, in each case combined with at least one compound d29; X, in each case combined with at least one compound d30; X, in each case combined with at least one compound d31; X, in each case combined with at least one compound d32; X, in each case combined with at least one compound d33; X, in each case combined with at least one compound d34; X, in each case combined with at least one compound d35; X, in each case combined with at least one compound d36; X, in each case combined with at least one compound d37; X, in each case combined with at least one compound d38; X, in each case combined with at least one compound d39; X, in each case combined with at least one compound d40; X, in each case combined with at least one compound d41; X, in each case combined with at least one compound d42; X, in each case combined with at least one compound d43; X, in each case combined with at least one compound d44; X, in each case combined with at least one compound d45; X, in each case combined with at least one compound d46; X, in each case combined with at least one compound d47; X, in each case combined with at least one compound d48; X, in each case combined with at least one compound d49; X, in each case combined with at least one compound d50; X, in each case combined with at least one compound d51; X, in each case combined with at least one compound d52; X, in each case combined with at least one compound d53; X, in each case combined with at least one compound d54; X, in each case combined with at least one compound d55; X, in each case combined with at least one compound d56; X, in each case combined with at least one compound d57; X, in each case combined with at least one compound d58; X, in each case combined with at least one compound d59; X, in each case combined with at least one compound d60; X, in each case combined with at least one compound d61; X, in each case combined with at least one compound d62; X, in each case combined with at least one compound d63; X, in each case combined with at least one compound d64; X, in each case combined with at least one compound d65; X, in each case combined with at least one compound d66; X, in each case combined with at least one compound d67; X, in each case combined with at least one compound d68; X, in each case combined with at least one compound d69; X, in each case combined with at least one compound d70; X, in each case combined with at least one compound d71; X, in each case combined with at least one compound d72; X, in each case combined with at least one compound d73; X, in each case combined with at least one compound d74; X, in each case combined with at least one compound d75; X, in each case combined with at least one compound d76; X, in each case combined with at least one compound d77; X, in each case combined with at least one compound d78; X, in each case combined with at least one compound d79; X, in each case combined with at least one compound d80; X, in each case combined with at least one compound d81; X, in each case combined with at least one compound d82; X, in each case combined with at least one compound d83; X, in each case combined with at least one compound d84; X, in each case combined with at least one compound d85; X, in each case combined with at least one module d86; X, in each case combined with at least one compound d87; X, in each case combined with at least one compound d88; X, in each case combined with at least one compound d89; X, in each case combined with at least one compound d90; X, in each case combined with at least one compound d91; X, in each case combined with at least one compound d92; X, in each case combined with at least one compound d93; X, in each case combined with at least one compound d94; X, in each case combined with at least one compound d95; X, in each case combined with at least one module d96; X, in each case combined with at least one compound d97; X, in each case combined with at least one compound d98; X, in each case combined with at least one compound d99; X, in each case combined with at least one compound d100; X, in each case combined with at least one compound d101; X, in each case combined with at least one compound d102; X, in each case combined with at least one compound d103; X, in each case combined with at least one compound d104; X, in each case combined with at least one compound d105; X, in each case combined with at least one compound d106; X, in each case combined with at least one compound d107; X, in each case combined with at least one compound d108; X, in each case combined with at least one compound d109; X, in each case combined with at least one compound d110; X, in each case combined with at least one compound d111; X, in each case combined with at least one compound d112; X, in each case combined with at least one compound d113; X, in each case combined with at least one compound d114; X, in each case combined with at least one compound d115; X, in each case combined with at least one compound d116; X, in each case combined with at least one compound d117; X, in each case combined with at least one compound d118; X, in each case combined with at least one compound d119; X, in each case combined with at least one compound d120; X, in each case combined with at least one compound d121; X, in each case combined with at least one compound d122; X, in each case combined with at least one compound d123; X, in each case combined with at least one compound d124; X, in each case combined with at least one compound d125; X, in each case combined with at least one compound d126; X, in each case combined with at least one compound d127; X, in each case combined with at least one compound d128; X, in each case combined with at least one compound d129; X, in each case combined with at least one compound d130; X, in each case combined with at least one compound d131; X, in each case combined with at least one compound d132; X, in each case combined with at least one compound d133; X, in each case combined with at least one compound d134; X, in each case combined with at least one compound d135; X, in each case combined with at least one compound d136; X, in each case combined with at least one compound d137; X, in each case combined with at least one compound d138; X, in each case combined with at least one compound d139; X, in each case combined with at least one compound d140; X, in each case combined with at least one compound d141; X, in each case combined with at least one compound d142; X, in each case combined with at least one compound d143; X, in each case combined with at least one compound d144; X, in each case combined with at least one compound d145; X, in each case combined with at least one compound d146; X, in each case combined with at least one compound d147; X, in each case combined with at least one compound d148; X, in each case combined with at least one compound d149; X, in each case combined with at least one compound d150; X, in each case combined with at least one compound d151; X, in each case combined with at least one compound d152; X, in each case combined with at least one compound d153; X, in each case combined with at least one compound d154; X, in each case combined with at least one compound d155; X, in each case combined with at least one compound d156; X, in each case combined with at least one compound d157; X, in each case combined with at least one compound d158; X, in each case combined with at least one compound d159; X, in each case combined with at least one compound d160; X, in each case combined with at least one compound d161; X, in each case combined with at least one compound d162; X, in each case combined with at least one compound d163; X, in each case combined with at least one compound d164; X, in each case combined with at least one compound d165; X, in each case combined with at least one compound d166; X, in each case combined with at least one compound d167; X, in each case combined with at least one compound d168; X, in each case combined with at least one compound d169; X, in each case combined with at least one compound d170;
wherein X is the [module (a)+module (b)+module (c)] protein or peptide and has the following meaning: abc1, abc2, abc3, abc4, abc5, abc6, abc7, abc8, abc9, abc10, abc11, abc12, abc13, abc14, abc15, abc16, abc17, abc18, abc19, abc20, abc21, abc22, abc23, abc24, abc25, abc26, abc27, abc28, abc29, abc30, abc31, abc32, abc33, abc34, abc35, abc36, abc37, abc38, abc39, abc40, abc41, abc42, abc43, abc44, abc45, abc46, abc47, abc48, abc49, abc50, abc51, abc52, abc53, abc54, abc55, abc56, abc57, abc58, abc59, abc60, abc61, abc62, abc63, abc64, abc65, abc66, abc67, abc68, abc69, abc70, abc71, abc72, abc73, abc74, abc75, or abc76, and wherein the [module (a)+module (b)+module (c)] protein or peptide and the at least one compound (d) are linked to each other in any arrangement and stoichiometry.

Particularly Preferred Multi-Module Conjugates of the Invention:

Particularly preferred conjugates of the present invention include: a CTB-COX peptide (SEQ ID NO: 2) conjugate comprising a17+bc1; a CTB-COX peptide (SEQ ID NO: 3) conjugate comprising a17+bc2; a CTB-mCTA conjugate comprising a17+bc4; an LT II A conjugate comprising (one of a33-a35)+bc6; a conjugate comprising abc1; a conjugate comprising abc2; a conjugate comprising abc3; a conjugate comprising abc4; a conjugate comprising abc5; a conjugate comprising abc6; a conjugate comprising abc7; a conjugate comprising abc8; a conjugate comprising abc9; a conjugate comprising abc10; a conjugate comprising abc11; a conjugate comprising abc12; a conjugate comprising abc13; a conjugate comprising abc14; a conjugate comprising abc15; a conjugate comprising abc16; a conjugate comprising abc17; a conjugate comprising abc18; a conjugate comprising abc19; a conjugate comprising abc20; a conjugate comprising abc21; a conjugate comprising abc22; a conjugate comprising abc23; a conjugate comprising abc24; a conjugate comprising abc25; a conjugate comprising abc26; a conjugate comprising abc27; a conjugate comprising abc28; a conjugate comprising abc29; a conjugate comprising abc30; a conjugate comprising abc31; a conjugate comprising abc32; a conjugate comprising abc33; a conjugate comprising abc34; a conjugate comprising abc35; a conjugate comprising abc36; a conjugate comprising abc37; a conjugate comprising abc38; a conjugate comprising abc39; a conjugate comprising abc40; a conjugate comprising abc41; a conjugate comprising abc42; a conjugate comprising abc43; a conjugate comprising abc44; a conjugate comprising abc45; a conjugate comprising abc46; a conjugate comprising abc47; a conjugate comprising abc48; a conjugate comprising abc49, a conjugate comprising abc50; a conjugate comprising abc51; a conjugate comprising abc52; a conjugate comprising abc53; a conjugate comprising abc54; a conjugate comprising abc55; a conjugate comprising abc56; a conjugate comprising abc57; a conjugate comprising abc58; a conjugate comprising abc59; a conjugate comprising abc60; a conjugate comprising abc61; a conjugate comprising abc62; a conjugate comprising abc63; a conjugate comprising abc64; a conjugate comprising abc65; a conjugate comprising abc66; a conjugate comprising abc67; a conjugate comprising abc68; a conjugate comprising abc69; a conjugate comprising abc70; a conjugate comprising abc71; a conjugate comprising abc72; a conjugate comprising abc73; a conjugate comprising abc74; a conjugate comprising abc75; and a conjugate comprising abc76. Preferably, these preferred conjugates of the invention further comprise at least one compound (d). More preferably, these preferred conjugates of the invention further comprise at least one compound (d) selected from the group consisting of a nucleic acid, a DNA molecule, an RNA molecule, a single stranded RNA molecule, a double stranded RNA molecule, an siRNA molecule, an shRNA molecule, a miRNA molecule, a protein, and a peptide.

In a third aspect, the present invention relates to methods of preparing a delivery system or conjugate of the invention. In a preferred embodiment, the method of preparing a conjugate of the invention comprises coupling (i.e., covalently or non-covalently linking, synthesizing, producing recombinantly, and the like) at least one module (a) that mediates cell targeting and facilitates cellular uptake, at least one module (b) that facilitates transport to the endoplasmic reticulum (ER), at least one module (c) that mediates translocation from the ER to the cytosol, and at least one compound (d), wherein the modules (a), (b) and (c) and the compound (d) are linked to each other in any arrangement and in any stoichiometry. The present invention also provides kits comprising at least one component of a conjugate of the invention. Preferably, a kit of the present invention comprises a module (a), a module (b), a module (c), and/or a compound (d). The kit optionally includes a peptide linker and/or a peptide comprising a cleavage site.

In a fourth aspect, the present invention relates to the use of the delivery system or conjugate of the present invention as a pharmaceutical.

In a fifth aspect, the present invention relates to a pharmaceutical composition comprising the conjugate of the present invention or a pharmaceutically acceptable salt thereof and a pharmaceutically acceptable excipient, carrier, and/or diluent. Preferably, the pharmaceutical composition comprises a pharmaceutically acceptable excipient, carrier and/or diluent and a conjugate of the present invention comprising at least one module (a), at least one module (b), at least one module (c) and at least one compound (d), wherein the modules (a), (b) and (c), and the compound (d) are linked to each other in any arrangement.

Suitable reactions for joining modules together:

To make the conjugates of the present invention, one can take advantage of the reactive functional groups that are available in naturally occurring proteins, such as primary amino groups that occur on the side chain of lysine (Lys) residues as well as the N-terminal amino group of the protein, and cysteine (Cys) thiols. Alternatively, peptides may be readily chemically synthesized that comprise or contain one or more additional functionalities such as alkynyl, aminooxy and hydrazino moieties that may be located at the terminus or internally in the synthesized peptide, for instance on the c-amino group of a Lys residue. For example, 6-BOC-HNA can be used to introduce an N-terminal aryl hydrazine and Fmoc-Lys-c-(6-BocHyNic) can be used to introduce an aryl hydrazine functionality internally in a peptide, and both of these reagents are available commercially (SoluLinK).

In a similar fashion, oligonucleotides such as DNA and RNA and their analogues can be synthesized with 5′-terminal, 3′-terminal or internal amino and thiol linkers and there are many suitable reagents commercially available for this purpose and can be employed to prepare the conjugates of the present invention. Furthermore, conjugates of the present invention can be prepared using commercially available reagents that are capable of performing bioorthogonal type conjugation reactions, such as the 4FB-phosphoramidite reagent (SoluLinK), which can be used to introduce a 5′-terminal benzaldehyde moiety, and a large number of alkyne containing compounds (see Glen Research, Base Click & ChemGenes), which can be used for “click-reactions”.

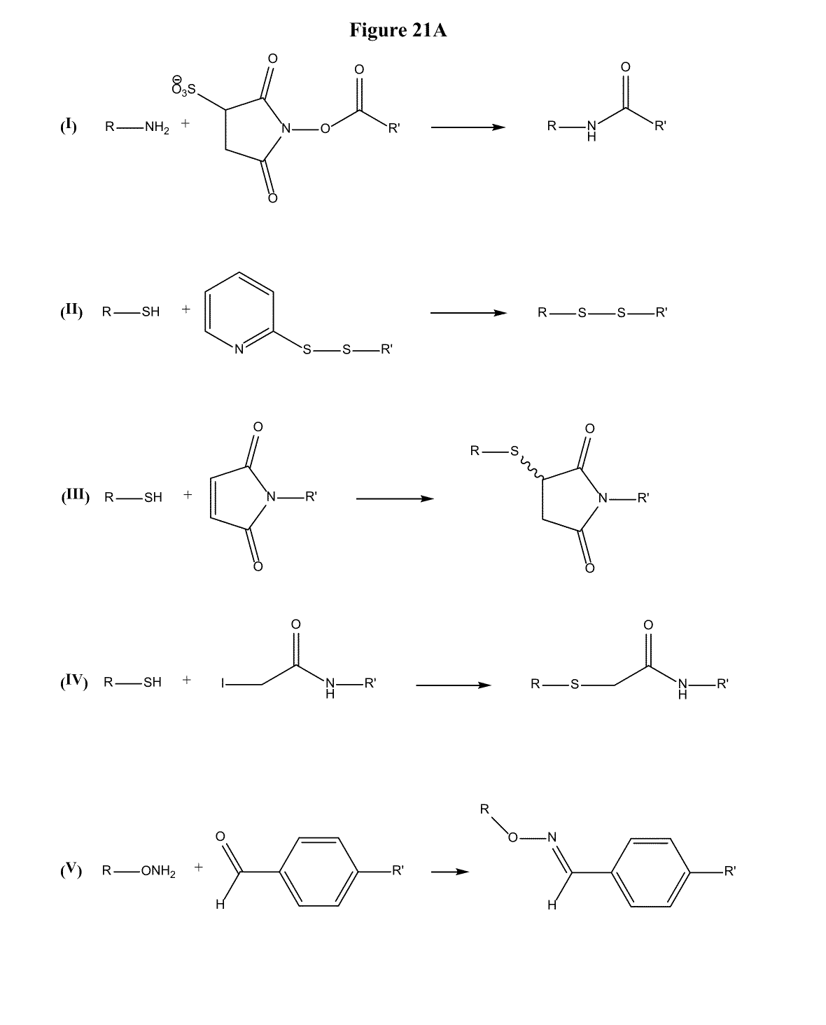

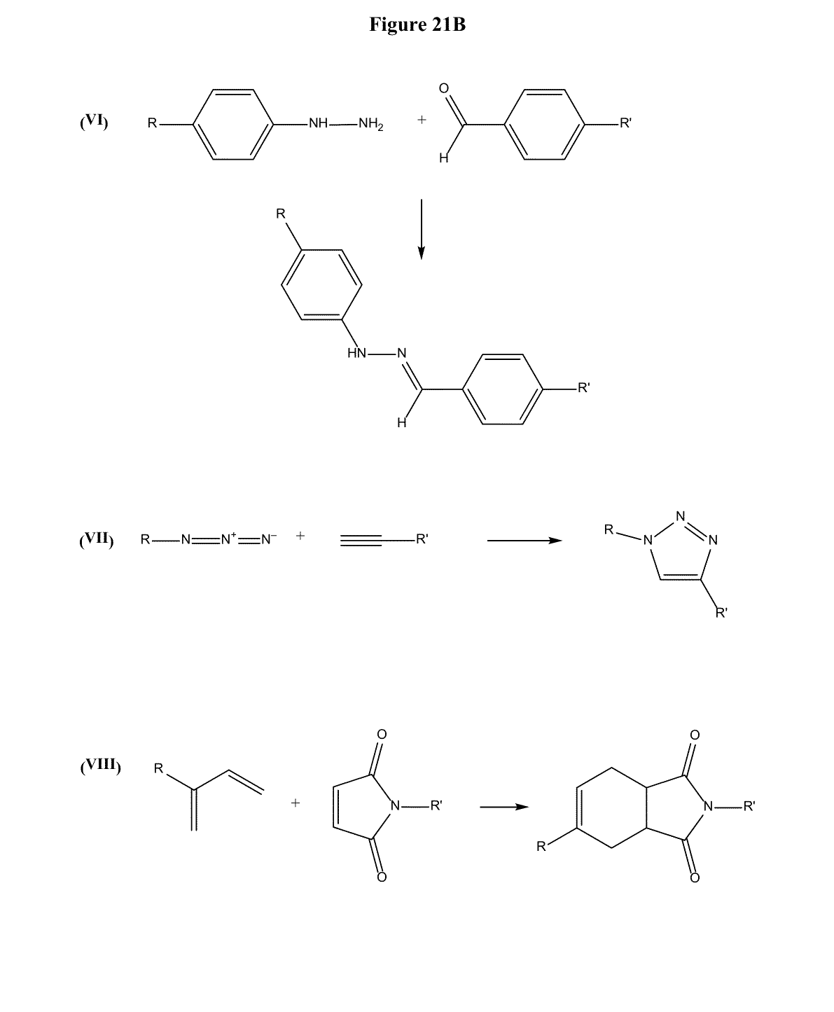

The various modules may be connected together in any order generally in a pairwise fashion using suitable direct coupling reactions between reaction pairs (see FIGS. 21A and 22B) which may or may not require the use of appropriate heterobifunctional crosslinkers to introduce the desired functionalities into the modules that should be connected. FIG. 22 describes eight (8) reaction pairs, all of which are eminently suitable for covalently linking the various modules of the conjugates of the present invention together. Thus, FIG. 21A panel (I) shows the reaction of a primary amine with a sulfo succinimidyl ester to generate a stable amide bond. FIG. 21A panel (II) shows the reaction of a thiol containing molecule with a 2-pyridyldisulfide comprising compound to give a product comprising a cleavable disulfide bond. FIGS. 22A panel (III) and 22A panel (IV) show the reactions of a thiol containing compound with a maleimide and an iodoacetamide, respectively, to give in both cases conjugates comprising stable thioether bonds. FIG. 21A panel (V) shows the reaction of an aminooxy containing compound with an aryl aldehyde to generate a stable aryl oxime. In the same class of reactions, FIG. 21B panel (VI) shows the reaction of an arylhydrazine with an aryl aldehyde to give a stable bis-arylhydrazone. FIG. 21B panel (VII) shows the reaction of an azide with an alkyne, which in the presence of Cu(I) catalysis, generates a very stable 1,2,3-triazine. This reaction is well known in the art as a “click-reaction”. FIG. 21B panel (VIII) shows a reaction, the Diels-Alder 4+2 cycloaddition, in which a suitable 1,3-diene reacts with a dienophile (illustrated here is a maleimide) to generate a cyclohexene ring. As drawn in the reaction schemes in FIGS. 21A and 22B, R′ may comprise a linker bearing at its terminus an orthogonal reactive functionality, in which case those compounds become heterobifunctional crosslinkers. Heterobifunctional crosslinkers and general protocols for their use are described in detail in Chapters 5 and 17 in Bioconjugate Techniques (2^{nd} edition, 2008, ed. Hermanson, G. T., Academic Press). Moreover, chapter 21 of this book shows how these protocols can be applied in the preparation of immunotoxin conjugates.

To make the conjugates of the present invention, one can employ a crosslinker to enable the connection of modules (a), (b), (c) and (d), such as:
Sulfo-LC-SPDP sulfosuccinimidyl 6-[3′-(2-pyridyldithio)-propionamido]hexanoate
Sulfo-LC-SMPT sulfosuccinimidyl-6-[α-methyl-α-(2-pyridyldithio)toluamido]hexanoate
Sulfo-SMCC sulfosuccinimidyl 4-(N-maleimidomethyl)-cyclohexane-1-carboxylate
Sulfo-GMBS N-(γ-maleimidobutyryloxy)sulfosuccinimide ester
Sulfo-SIAB sulfosuccinimidyl (4-iodoacetylamino)benzoate
Sulfo-S-4FB sulfosuccinimidyl 4-formylbenzoate (SoluLinK)
S-SS-4FB similar to S-4FB but has an internal disulfide bond (SoluLinK)
Sulfo-S-HyNic sulfosuccinimidyl 6-hydrazinonicotinate acetone hydrazone (SoluLinK)
M₂C₂H 4-(N-maleimidomethyl)cyclohexane-1-carboxylhydrazide hydrochloride
PDPH 3-(2-pyridyldithio)propionyl hydrazide

For a module (a) protein or peptide that comprises a free thiol moiety, it may be attached to a linkage molecule comprising module (b)+module (c)+linker via Method A as exemplified by Example (1) section (ii) below. Examples of module (a) proteins and peptides that fall into this class include the B subunits of the plant toxins ricin, abrin, modeccin, volkensin and viscumin.

In the absence of a free accessible thiol moiety on a module (a) protein or peptide, then Method B [Example 2, sections (ii) and (iii)] can be employed, which involves reacting a heterobifunctional crosslinker with an accessible amino group, this will generally be the N-terminal amino group or a lysine side chain amino group on the surface of module (a) protein or peptide. Suitable heterobifunctional crosslinkers include sulfo-LC-SPDP and sulfo-LC-SMPT amongst others. An alternative heterobifunctional linker that does not result in a disulfide bond is sulfo-SMCC. The amount of the heterobifunctional crosslinker should be controlled, preferably within the range of about 4-10 molar excess, so as to achieve the desired level of functionalisation of the module (a) protein or peptide. Examples of B-subunit bacterial toxins that fall into the Method B category are the homopentamers of cholera toxin B-subunit (CTB), Shiga toxin B-subunit, and the heteropentamer of Pertussis toxin B-subunit. A synthesis protocol for CTB is illustrated in Example (2) section (ii) below.

In one embodiment, the conjugates of the delivery system of the present invention exclude the following compounds: The conjugates according to FIG. 2A, FIG. 2B, FIG. 4, FIG. 5, FIG. 6A, FIG. 6B, FIG. 7, FIG. 8, FIG. 9, FIG. 10A, FIG. 10B, FIG. 11, FIG. 12, FIG. 13, FIG. 14, (Ricin B)-S—S-Cys(NH₂)-(Ser-Gly)₃-NH—CR₁—C(═O)-(Ser-Gly)₃-AsnAlaSerSerSerArgSerGlyLeuAspAspIleAsnProThrValLeuLeuLysGluArgSerThrGluLeu-OH (Ricin B comprising SEQ ID NO: 115 and CX1 peptide according to SEQ ID NO: 2) or (Ricin B)-S—S-Cys(NH₂)-(Ser-Gly)₃-NH—CR₁—C(═O)-(Ser-Gly)-3-Asn-AlaSerSerSerArgSerGly-LeuAspAspIleAsnProThrValLeuLeuLysAlaLysAspGluLeu-OH (Ricin B comprising SEQ ID NO: 115 and CX2a peptide according to SEQ ID NO: 3), (Ricin B)-S—S-Cys(NH₂)-(dPEG12)-NH—CR₁—C(═O)-(dPEG12)-AsnAlaSerSerSerArgSerGlyLeuAspAspIleAsnProThr-ValLeuLeuLysGluArg-SerThrGluLeu-OH (Ricin B comprising SEQ ID NO: 115 and CX1 peptide according to SEQ ID NO: 2), H₂N-HisLeuAsnIleLeuSerThrLeuTrpLysTyrArg-(Linker)-Cys-S—S-Cys-(dPEG12)-NH—CR₁—C(═O)-(dPEG12)-AsnAlaSerSerSerArgSerGlyLeuAsp-Asp-IleAsnProThrValLeuLeuLysAlaLysAspGluLeu-OH (Tet1 peptide according to SEQ ID NO: 190 and CX2a peptide according to SEQ ID NO: 3, GlnValGlnLeuValGluSerGlyGlyGlyLeuValGlnProGlyGlySerLeuArgLeuProCysAlaAlaSerGly SerIlePheSerLeuAspAlaTrpGlyTrpTyrArgGlnAlaProGlyLysGlnArgGluMetValAlaLeuValGly-SerAspGlySerThrSerTyrAlaAspSerValLysGlyArgPheThrIleSerArgAspAsnAlaAsnAsnThrPhe TyrLeuGlnMetAsnSerLeuLysProGluAspThrAlaValTyrTyrCysTyrAlaArgPheGlnSerLeuTyr-AsnSerTrpGlyGlnGlyThrGlnValThrValSerSerCys-S—S-Cys-(dPEG12)-NH—CR₁—C(═O)-(dPEG12)-AsnAlaSerSerSerArgSerGlyLeuAspAspIleAsnProThrValLeuLeuLysAlaLys-AspGluLeu-OH (anti EGFR single chain antibody according to SEQ ID NO: 196 and CX2a peptide according to SEQ ID NO: 3)

wherein linker is either Gly-Gly-Gly, Ser-GlySer-Gly or Ser-Gly-Ser-Gly-Ser-Gly and R₁ is —CH₂—NH—C(═O)—CH₂—O—N═CH-(para-Phenyl)-C(═O)—NH—(CH₂)₆—O—(PO₂)⁻O—(CH₂)₆—S—S—(CH₂)₆—O—(PO₂)⁻—O—Cy3-siRNA, Human serum trans ferrin-O—(CH₂)₁₂-(dPEG12)-NH—CR₂-(dPEG12)-AsnAlaSerSerSerArgSerGlyLeuAspAspIleAsnProThrValLeuLeuLysAlaLysAspGluLeu-OH (Human serum transferin according to SEQ ID NO: 191 and CX2a peptide according to SEQ ID NO: 3), wherein R₂ is —(CH₂)₄—NH-(dPEG12)-Cys-S-Cys (NH₂)-PhePheMetGluGluLeuAsnThrTyrArgGln-LysGlnGlyValValLeuLysTyrGln-GluLeuProAsnSer-GlyProProHisAspArgArgPheThrPheGln-ValIleIleAspGlyArgGluPheProGluGlyGluGlyArgSerLysLysGluAlaLysAsnAlaAlaAla-LysLeu-AlaValGluIleLeuAsnLysGlu-OH (SEQ ID NO: 192), —(CH₂)₄—NH-(dPEG12)-Cys-S-Cys(OH)-GluLysAsnLeuIleGluValAlaLeuLysAlaAlaAla-Asn-Lys-AlaGluLysLysSerArgGlyGluGlyGluPro-PheGluArgGlyAspIleIleValGlnPheThrPheArg-Arg-AspHisProProGlySerAsnProLeuGluGlnTyr-LysLeuValValGlyGlnLysGlnArgTyrThrAsn-LeuGluGluMetPhePhe-NH₂ (SEQ ID NO: 193), or

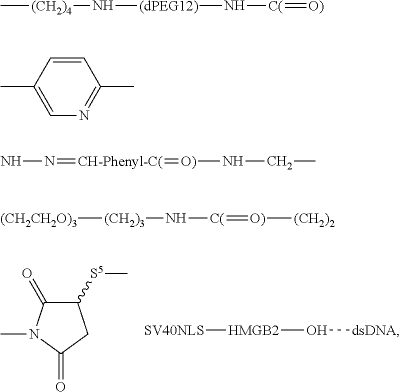

wherein the dashed line indicates non-covalent bond(s) to the dsDNA. In a further embodiment also the constructs of FIG. 3 are excluded.

Any conjugate of the present invention may be admixed with a pharmaceutically acceptable excipient, carrier, or diluent, or a mixture thereof. Even though the conjugates of the present invention (including their pharmaceutically acceptable salts, esters and pharmaceutically acceptable solvates) can be administered alone, they will generally be administered in admixture with a pharmaceutical buffer, diluent, or excipient, particularly for human therapy.

The term “excipient” when used herein is intended to indicate all substances in a pharmaceutical formulation which are not active ingredients such as, e.g., binders, lubricants, thickeners, surface active agents, preservatives, emulsifiers, buffers, decharging agents, flavoring agents, or colorants. Examples of such suitable excipients for the various different forms of pharmaceutical compositions described herein have been previously described [51]. Preferably, to neutralize the high negative charge of the nucleic acids within a conjugate of the present invention, human protamine, spermine, spermidine or other polycations, can be added to the conjugate or a formulation of the conjugate of the present invention.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s). Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol. Examples of suitable lubricants include sodium oleate, sodium stearate, magnesium stearate, sodium benzoate, sodium acetate, sodium chloride and the like. Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

As used herein, “pharmaceutically acceptable carrier” includes any material, which when combined with the conjugate retains the activity of the conjugate activity and is non-reactive with the subject's immune system. Examples include, but are not limited to, any of the standard pharmaceutical carriers such as a phosphate buffered saline solution, water, emulsions such as oil/water emulsion, glycerol, ethanol, and various types of wetting agents. Other carriers may also include sterile solutions, tablets including coated tablets and capsules. Typically such carriers contain excipients such as starch, milk, sugar, glucose, lactose, certain types of clay, gelatin, stearic acid or salts thereof, methyl cellulose, magnesium stearate, mannitol, sorbitol, magnesium or calcium stearate, talc, vegetable fats or oils, gums, glycols, or other known excipients. Such carriers may also include flavor and color additives or other ingredients. Compositions comprising such carriers are formulated by well known conventional methods.

The term “pharmaceutically acceptable salt” refers to a salt of the conjugate of the present invention. Suitable pharmaceutically acceptable salts include acid addition salts which may, for example, be formed by mixing a solution of the conjugate of the present invention with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Illustrative examples of pharmaceutically acceptable salts include, but are not limited to, acetate, adipate, alginate, ascorbate, aspartate, benzenesulfonate, benzoate, bicarbonate, bisulfate, bitartrate, borate, bromide, butyrate, calcium edetate, camphorate, camphorsulfonate, camsylate, carbonate, chloride, citrate, clavulanate, cyclopentanepropionate, digluconate, dihydrochloride, dodecylsulfate, edetate, edisylate, estolate, esylate, ethanesulfonate, formate, fumarate, gluceptate, glucoheptonate, gluconate, glutamate, glycerophosphate, glycolylarsanilate, hemisulfate, heptanoate, hexanoate, hexylresorcinate, hydrabamine, hydrobromide, hydrochloride, hydroiodide, 2-hydroxy-ethanesulfonate, hydroxynaphthoate, iodide, isothionate, lactate, lactobionate, laurate, lauryl sulfate, malate, maleate, malonate, mandelate, mesylate, methanesulfonate, methylsulfate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, 3-phenylpropionate, phosphate/diphosphate, picrate, pivalate, polygalacturonate, propionate, salicylate, sodium, stearate, sulfate, subacetate, succinate, tannate, tartrate, teoclate, tosylate, triethiodide, undecanoate, valerate, and the like [see, for example, 52]. When compound (d) of a conjugate of the present invention is a nucleic acid, the pharmaceutically acceptable salt is preferably a sodium salt.

Pharmaceutical compositions of the invention are suitable for use in a variety of drug delivery systems. Suitable formulations for use in the present invention, including acceptable carrier or diluents for therapeutic use are well known in the pharmaceutical art, and methods for drug delivery are described (see for example [53 and 54]).

The pharmaceutical compositions may be formulated for any appropriate manner of administration to an organism, preferably a mammal, and even more preferably a human. As used herein, “administering” includes topical, transdermal, intradermal, oral, nasal, inhalation, transmucosal, intravenous, intra-arterial, intravascular, intracardiac, intraosseous, intrathecal, intracranial, epidural, intracerebral, intracerebroventricular, intracisternal, intraperitoneal, intralesional, intravesical, intravitreal, intracaverous, intravaginal, vaginal, intrauterine, rectal, subcutaneous or intramuscular administration and the means or the implantation of a slow-release device e.g., an osmotic pump, to the subject. The concentration of a conjugate of the present invention in the pharmaceutical composition will vary upon the particular application, the nature of the disease, the frequency of administration, or the like.

Commonly, the pharmaceutical compositions are administered parenterally, e.g., intravenously. Thus, the invention provides pharmaceutical compositions for parenteral administration that comprise the conjugate of the present invention dissolved or suspended in an acceptable carrier, preferably an aqueous carrier, e.g., water, buffered water, saline, PBS, alcohol, and the like. The pharmaceutical compositions may further comprise pharmaceutically acceptable auxiliary substances as required to approximate physiological conditions, such as pH adjusting and buffering agents, tonicity adjusting agents, wetting agents, detergents and the like.

These pharmaceutical compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably from 5 to 9 and most preferably from 7 and 8.

In some embodiments, the conjugates of the invention can be incorporated into liposomes formed from standard vesicle-forming lipids. A variety of methods are available for preparing liposomes, as described in, e.g., [55-57]; U.S. Pat. Nos. 4,235,871, 4,501,728 and 4,837,028. The targeting of liposomes using a variety of targeting agents is well known in the art (see, e.g., U.S. Pat. Nos. 4,957,773 and 4,603,044). Standard methods for coupling targeting agents to liposomes can be used. These methods generally involve incorporation into liposomes of lipid components, such as phosphatidylethanolamine, which can be activated for attachment of targeting agents, or derivatized lipophilic compounds, such as lipid-derivatized peptides of the invention. Targeting mechanisms generally require that the targeting agents be positioned on the surface of the liposome in such a manner that the target moieties are available for interaction with the target, for example, a cell surface receptor. Commonly used lipid delivery methods that are used to deliver siRNAs have been previously described and may be of use with the conjugates of the present invention [58-61].

In a preferred embodiment, a conjugate of the present invention, particularly wherein the conjugate comprises an siRNA as compound (d), is administered in vivo using a method currently used for therapeutic siRNAs. Such methods include but are not limited to cholesterol conjugation to the conjugate, the use of polycation nanoparticles to deliver the conjugate to a target cell via a cell surface ligand that binds to a receptor on the target cell, encapsulation of the conjugate into a cationic or neutral lipid bilayer using SNALPs (stable nucleic acid lipid particles) that are coated with diffusible PEG-lipid conjugates, masked endosomolytic agent (MEA)-dynamic polyconjugates (DPCs) comprising a ligand to target the conjugate to a specific cell, the use of protamine-tagged (or any other positive charged molecule-tagged) specific antibody to target the conjugate to a specific cell for receptor-mediated uptake, the use of RNA aptamers to target the conjugate to a specific cell, the use of immunoliposomes, Trans-IT TKO, LF2000, and the like [62-64].

The dosage ranges for the administration of the conjugates of the invention are those large enough to produce the desired effect in which the symptoms of the disease or condition to be treated show some degree of amelioration. The dosage should not be so large as to cause adverse side effects. Generally, the dosage will vary with the age, condition, sex and extent of the disease in a subject or patient and can be determined by one of skill in the art. Dosage regimens are adjusted to provide the optimum desired response (e.g., a therapeutic response). For example, a single bolus may be administered, several divided doses may be administered over time or the dose may be proportionally reduced or increased as necessitated by the therapeutic situation. It is especially advantageous to formulate parenteral compositions in dosage unit form for ease of administration and uniformity of dosage.

Preferably, the conjugates of the present invention are administered intravenously at a dose ranging from about 1 to about 4000 nmol/kg, from about 1 to about 3000 nmol/kg, from about 1 to about 2000 nmol/kg, from about 1 to about 1000 nmol/kg, from about 100 to about 4000 nmol/kg, from about 100 to about 3000 nmol/kg, from about 100 to about 2000 nmol/kg, from about 100 to about 1000 nmol/kg, from about 200 to about 4000 nmol/kg, from about 200 to about 3000 nmol/kg, from about 200 to about 2000 nmol/kg, from about 200 to about 1000 nmol/kg, from about 300 to about 4000 nmol/kg, from about 300 to about 3000 nmol/kg, from about 300 to about 2000 nmol/kg, from about 300 to about 1000 nmol/kg, from about 500 to about 4000 nmol/kg, from about 500 to about 3000 nmol/kg, from about 500 to about 2000 nmol/kg, from about 500 to about 1000 nmol/kg, from about 1000 to about 4000 nmol/kg, from about 1000 to about 3000 nmol/kg, from about 1000 to about 2000 nmol/kg, from about 2000 to about 4000 nmol/kg, from about 2000 to about 3000 nmol/kg, from about 3000 to about 4000 nmol/kg, from about 1 to about 500 nmol/kg, from about 1 to about 400 nmol/kg, from about 1 to about 300 nmol/kg, from about 1 to about 200 nmol/kg, from about 1 to about 100 nmol/kg, from about 10 to about 500 nmol/kg, from about 10 to about 400 nmol/kg, from about 10 to about 300 nmol/kg, from about 10 to about 200 nmol/kg, from about 10 to about 100 nmol/kg, from about 100 to about 500 nmol/kg, from about 100 to about 400 nmol/kg, from about 100 to about 300 nmol/kg, from about 100 to about 200 nmol/kg, from about 200 to about 500 nmol/kg, from about 200 to about 400 nmol/kg, from about 200 to about 300 nmol/kg, from about 300 to about 500 nmol/kg, from about 300 to about 400 nmol/kg, from about 400 to about 500 nmol/kg, from about 1 to about 50 nmol/kg, from about 1 to about 40 nmol/kg, from about 1 to about 30 nmol/kg, from about 1 to about 20 nmol/kg, from about 1 to about 10 nmol/kg, from about 1 to about 5 nmol/kg, from about 1 to about 4 nmol/kg, from about 1 to about 3 nmol/kg, from about 1 to about 2 nmol/kg, from about 2 to about 5 nmol/kg, from about 2 to about 4 nmol/kg, from about 2 to about 3 nmol/kg, from about 3 to about 5 nmol/kg, or from about 3 to about 4 nmol/kg.

Preferably, the conjugates of the present invention are administered intracranially or via an osmotic pump at a dose ranging from about 0.001 to about 10 nmol, from about 0.001 to about 5 nmol, from about 0.001 to about 3 nmol, from about 0.001 to about 2 nmol, from about 0.001 to about 1 nmol, from about 0.001 to about 0.5 nmol, from about 0.001 to about 0.3 nmol, from about 0.001 to about 0.2 nmol, from about 0.001 to about 0.1 nmol, from about 0.001 to about 0.05 nmol, from about 0.001 to about 0.03 nmol, from about 0.001 to about 0.02 nmol, from about 0.001 to about 0.01 nmol, from about 0.001 to about 0.005 nmol, from about 0.001 to about 0.003 nmol, from about 0.001 to about 0.002 nmol, from about 0.002 to about 10 nmol, from about 0.002 to about 5 nmol, from about 0.002 to about 3 nmol, from about 0.002 to about 2 nmol, from about 0.002 to about 1 nmol, 0.002 to about 0.5 nmol, from about 0.002 to about 0.3 nmol, from about 0.002 to about 0.2 nmol, from about 0.002 to about 0.1 nmol, from about 0.002 to about 0.05 nmol, from about 0.002 to about 0.03 nmol, from about 0.002 to about 0.02 nmol, from about 0.002 to about 0.01 nmol, from about 0.002 to about 0.005 nmol, from about 0.002 to about 0.003 nmol, from about 0.003 to about 10 nmol, from about 0.003 to about 5 nmol, from about 0.003 to about 3 nmol, from about 0.003 to about 2 nmol, from about 0.003 to about 1 nmol, 0.003 to about 0.5 nmol, from about 0.003 to about 0.3 nmol, from about 0.003 to about 0.2 nmol, from about 0.003 to about 0.1 nmol, from about 0.003 to about 0.05 nmol, from about 0.003 to about 0.03 nmol, from about 0.003 to about 0.02 nmol, from about 0.003 to about 0.01 nmol, from about 0.003 to about 0.005 nmol, from about 0.005 to about 10 nmol, from about 0.005 to about 5 nmol, from about 0.005 to about 3 nmol, from about 0.005 to about 2 nmol, from about 0.005 to about 1 nmol, 0.005 to about 0.5 nmol, from about 0.005 to about 0.3 nmol, from about 0.005 to about 0.2 nmol, from about 0.005 to about 0.1 nmol, from about 0.005 to about 0.05 nmol, from about 0.005 to about 0.03 nmol, from about 0.005 to about 0.02 nmol, from about 0.005 to about 0.01 nmol, from about 0.01 to about 10 nmol, from about 0.01 to about 5 nmol, from about 0.01 to about 3 nmol, from about 0.01 to about 2 nmol, from about 0.01 to about 1 nmol, from about 0.01 to about 0.5 nmol, from about 0.01 to about 0.3 nmol, from about 0.01 to about 0.2 nmol, from about 0.01 to about 0.1 nmol, from about 0.01 to about 0.05 nmol, from about 0.01 to about 0.03 nmol, from about 0.01 to about 0.02 nmol, from about 0.02 to about 10 nmol, from about 0.02 to about 5 nmol, from about 0.02 to about 3 nmol, from about 0.02 to about 2 nmol, from about 0.02 to about 1 nmol, from about 0.02 to about 0.5 nmol, from about 0.02 to about 0.3 nmol, from about 0.02 to about 0.2 nmol, from about 0.02 to about 0.1 nmol, from about 0.02 to about 0.05 nmol, from about 0.02 to about 0.03 nmol, from about 0.03 to about 10 nmol, from about 0.03 to about 5 nmol, from about 0.03 to about 3 nmol, from about 0.03 to about 2 nmol, from about 0.03 to about 1 nmol, from about 0.03 to about 0.5 nmol, from about 0.03 to about 0.3 nmol, from about 0.03 to about 0.2 nmol, from about 0.03 to about 0.1 nmol, from about 0.03 to about 0.05 nmol, from about 0.05 to about 10 nmol, from about 0.05 to about 5 nmol, from about 0.05 to about 3 nmol, from about 0.05 to about 2 nmol, from about 0.05 to about 1 nmol, from about 0.05 to about 0.5 nmol, from about 0.05 to about 0.3 nmol, from about 0.05 to about 0.2 nmol, from about 0.05 to about 0.1 nmol, from about 0.1 to about 10 nmol, from about 0.1 to about 5 nmol, from about 0.1 to about 3 nmol, from about 0.1 to about 2 nmol, from about 0.1 to about 1 nmol, from about 0.1 to about 0.5 nmol, from about 0.1 to about 0.3 nmol, from about 0.1 to about 0.2 nmol, from about 0.2 to about 10 nmol, from about 0.2 to about 5 nmol, from about 0.2 to about 3 nmol, from about 0.2 to about 2 nmol, from about 0.2 to about 1 nmol, from about 0.2 to about 0.5 nmol, from about 0.2 to about 0.3 nmol, from about 0.3 to about 10 nmol, from about 0.3 to about 5 nmol, from about 0.3 to about 3 nmol, from about 0.3 to about 2 nmol, from about 0.3 to about 1 nmol, from about 0.3 to about 0.5 nmol, from about 0.5 to about 10 nmol, from about 0.5 to about 5 nmol, from about 0.5 to about 3 nmol, from about 0.5 to about 2 nmol, from about 0.5 to about 1 nmol, from about 1 to about 10 nmol, from about 1 to about 5 nmol, from about 1 to about 3 nmol, from about 1 to about 2 nmol, from about 2 to about 10 nmol, from about 2 to about 5 nmol, from about 2 to about 3 nmol, from about 3 to about 10 nmol, from about 3 to about 5 nmol, or from about 5 to about 10 nmol.

More preferably, the conjugates of the invention, when administered via an osmotic pump, are administered at a daily dose of about 3 nmol.

Additional pharmaceutical methods may be employed to control the duration of action. Controlled release preparations may be achieved by the use of polymers to conjugate, complex or adsorb the conjugates of the present invention. The controlled delivery may be exercised by selecting appropriate macromolecules (for example, polyesters, polyamino carboxymethylcellulose, and protamine sulfate) and the concentration of macromolecules as well as the methods of incorporation in order to control release. Another possible method to control the duration of action by controlled release preparations is to incorporate the conjugate into particles of a polymeric material such as polyesters, polyamino acids, hydrogels, poly(lactic acid) or ethylene vinylacetate copolymers.

In order to protect the conjugates of the present invention, and the peptides or proteins comprised within said conjugates, from binding with plasma proteins, it is preferred that the conjugates be entrapped in microcapsules prepared, for example, by coacervation techniques or by interfacial polymerization, for example, hydroxymethylcellulose or gelatin-microcapsules and poly(methymethacrylate) microcapsules, respectively, or in colloidal drug delivery systems, for example, liposomes, albumin microspheres, microemulsions, nanoparticles, and nanocapsules or in macroemulsions. Such teachings have been previously described [53].

The conjugates of the invention are well suited for use in targetable drug delivery systems such as synthetic or natural polymers in the form of macromolecular complexes, nanocapsules, microspheres, or beads, meso-particles, and lipid-based systems including oil-in-water emulsions, micelles, mixed micelles, liposomes, and resealed erythrocytes. These systems are known collectively as colloidal drug delivery systems. Typically, such colloidal particles containing the dispersed conjugates are about 50 nm-2 μm in diameter. The size of the colloidal particles allows them to be administered intravenously such as by injection, or as an aerosol. Materials used in the preparation of colloidal systems are typically sterilizable via filter sterilization, nontoxic, and biodegradable, for example albumin, ethylcellulose, casein, gelatin, lecithin, phospholipids, and soybean oil. Polymeric colloidal systems are prepared by a process similar to the coacervation of microencapsulation. The targeted delivery system-encapsulated conjugate may be provided in a formulation comprising other compounds as appropriate and an aqueous physiologically acceptable medium, for example, saline, phosphate buffered saline, or the like.

In an exemplary embodiment, the conjugates of the present invention are components of a liposome, used as a targeted delivery system. When phospholipids are gently dispersed in aqueous media, they swell, hydrate, and spontaneously form multilamellar concentric bilayer vesicles with layers of aqueous media separating the lipid bilayer. Such systems are usually referred to as multilamellar liposomes or multilamellar vesicles (MLVs) and have diameters ranging from about 100 nm to about 4 μm. When MLVs are sonicated, small unilamellar vesicles (SUVS) with diameters in the range of from about 20 nm to about 50 nm are formed, which contain an aqueous solution in the core of the SUV.

Examples of lipids useful in liposome production include phosphatidyl compounds, such as phosphatidylglycerol, phosphatidylcholine, phosphatidylserine, and phosphatidylethanol-amine Particularly useful are diacylphosphatidylglycerols, wherein the lipid moiety comprises from 14-18 carbon atoms, particularly from 16-18 carbon atoms, and are saturated. Illustrative phospholipids include egg phosphatidylcholine, dipalmitoylphosphatidylcholine, and distearoylphosphatidylcholine.

In a sixth aspect, the conjugates of the present invention may be of use as diagnostic reagents. For example, labeled compounds can be used to locate areas of inflammation or tumor metastasis in a patient suspected of having an inflammation. For this use, the compounds can be labeled with ¹²⁵I, ¹⁴C, or tritium.

In a seventh aspect, the present invention relates to the use of the delivery system or conjugate of the invention for the manufacture of a medicament (i.e., a pharmaceutical composition). The pharmaceutical compositions may be used to treat humans or animals, in human and veterinary medicine respectively.

In an eighth aspect, the present invention relates to a method of delivering the compound (d) to a cell, which comprises the steps:
(a) providing a cell,
(b) contacting a conjugate of the present invention with said cell,
   under the conditions that allow the conjugate to be internalized by the cell, thereby delivering compound (d) to the cell. In one embodiment, the cell is an isolated cell or cultured cell.

Preferably, the cell is a eukaryotic cell, an invertebrate cell, a vertebrate cell, a nematode cell, a fungal cell, an Aspergillus cell, a yeast cell, a Sacchromyces cell, a Pichia cell, an insect cell, an Sf9 cell, an animal cell, a non-human animal cell, a Chinese hamster ovary (CHO) cell, a mammalian cell, a non-human mammalian cell, a primate cell, a non-human primate cell, a human cell, or a plant cell. In a preferred embodiment, the method of delivering a compound (d) to a cell results in increased or decreased gene expression and/or protein production in the cell.

In a particularly preferred embodiment, the method of delivering a compound (d) to a cell comprises the steps:
(a) providing a cell,
(b) contacting a conjugate of the present invention with said cell,
   under the conditions that allow the conjugate to be internalized by the cell, thereby delivering compound (d), and whereby gene expression of said cell is modified (i.e., increased or decreased) and/or protein production in said cell is modified (i.e., increased or decreased). Thus, methods of modifying gene expression and/or protein production in a cell using the delivery system or conjugate of the present invention are also provided. Preferably, the cell is an isolated cell or a cultured cell. More preferably, the cell is an isolated cell or cultured cell used for recombinant gene expression, protein production, and/or drug, small molecule, or biological molecule screening. Preferably, the isolated cell or cultured cell is a eukaryotic cell, an invertebrate cell, a vertebrate cell, a nematode cell, a fungal cell, an Aspergillus cell, a yeast cell, a Sacchromyces cell, a Pichia cell, an insect cell, an insect cell, an animal cell, a non-human animal cell, a CHO cell, a mammalian cell, a non-human mammalian cell, a primate cell, a non-human primate cell, a human cell, or a plant cell.

In a ninth aspect, the present invention relates to a method of delivering a compound (d) to an organism comprising the step of administering a sufficient amount of a conjugate of the present invention to a patient, thereby delivering the compound (d) to the organism.

Preferably, the organism is an animal, a mammal, a human, or a plant. In a preferred embodiment, the method of delivering a compound (d) to an organism results in increased or decreased gene expression and/or protein production in a cell of the organism. In another preferred embodiment, the method of delivering a compound (d) to an organism results in increased immunity or an increased immune response in the organism.

In a tenth aspect, the present invention relates to a method of delivering a compound (d) to a patient comprising the step of administering a sufficient amount of a conjugate of the present invention to a patient, thereby delivering the compound (d) to the patient.

As used herein, a “patient” refers to an organism suffering from and/or undergoing treatment for a disorder, disease or condition. The patient can be any animal but is preferably a mammal, such as a cow, horse, mouse, rat, cat, dog, pig, goat, sheep, chicken, or a primate. In a preferred embodiment, the patient is a human. Preferably, the patient is an animal, a non-human animal, a mammal, a non-human mammal, or a human. More preferably, the patient is a human suffering from and/or undergoing treatment for a disorder, disease or condition mediated by increased, decreased, insufficient, aberrant or unwanted target gene expression or protein production. In an another embodiment, the patient is suffering from and/or undergoing treatment for a disorder, disease or condition mediated by decreased, insufficient, or lack of immunity.

In a preferred embodiment, a method of delivering a compound (d) to a patient comprises the step of administering to a patient a sufficient amount of a conjugate comprising, essentially consisting of or consisting of:
(a) at least one module (a) that mediates cell targeting and facilitates cellular uptake,
(b) at least one module (b) that facilitates transport to the endoplasmic reticulum (ER),
(c) at least one module (c) that mediates translocation from the ER to the cytosol, and
(d) at least one compound (d),
   wherein the modules (a), (b) and (c), and the compound (d) are linked to each other in any arrangement, and wherein the conjugate optionally comprises a nuclear localization signal, and thereby delivering the compound (d) to the patient.

Preferably, the compound (d) to be delivered to a patient using a method according to the invention is an siRNA.

In a further aspect, the present invention relates to the conjugates of the present invention for use in therapy and prevention of disease, which can be prevented or treated by the delivery of at least one compound (d).

A “disease” is a state of health of an organism, wherein the organism cannot maintain homeostasis, and wherein if the disease is not ameliorated then the organism's health begins or continues to deteriorate.

Because RNAi mediated silencing is expected to persist for several days after administering a conjugate according to the invention comprising an siRNA as compound (d), in many instances, it is possible to administer the conjugates of the present invention with a frequency of less than once per day, or, for some instances, only once for the entire therapeutic regimen. For example, treatment of some cancer cells may be mediated by a single bolus administration, whereas a chronic viral infection may require regular administration, e.g., once per week or once per month.

The present invention provides conjugates which can effectively deliver compounds such as biologically active macromolecules, nucleic acids or peptides in particular, to a cell, either in culture or within an organism by using endogenous processes that occur ubiquitously within all cells.

Various modifications and variations of the invention will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be encompassed by the present invention.

The invention is now described with reference to the following Examples. These Examples are provided for the purpose of illustration only and the invention should in no way be construed as being limited to these Examples, but rather should be construed to encompass any and all variations which become evident as a result of the teaching provided herein.

### EXAMPLES

Abbreviations used herein include: kilogram (kg), milligram (mg), milliliter (mL), microliter (μL), molar (M), millimolar (mM), micromolar (μM), micromoles (μmol), nanomoles (nmol), hour (h), kiloDalton (kDa), degrees Celsius (° C.), minute (min), millimeter (mm), micron (μm), nanometer (nm), amino acid (aa), wild-type (wt), gravity (g), and intraperitonea1 (i.p.).

### Example (1)

### Synthesis of DARE™ Delivery System Delivery Modules and Preparation of the Modules-siRNA Conjugate DARE™-RTB-COX2-KDEL-siRNA (DARE™ Delivery Vehicle Design 2.03)

(i) Synthesis of the Linkage Molecule Containing Delivery Modules (b) and (c):

A [“module (b)+module (c)”+linker] molecule: H₂N—C(NPyS)(S-G)₃(DprAoa)(S-G)₃NASSSRSGLDDINPTVLLKAKDEL-OH [“module (b)+module (c)” comprise SEQ ID NO: 3; COX2-KDEL] is synthesized commercially by standard solid-phase Fmoc peptide chemistry, deprotected in the standard fashion and purified by reversed phase High Performance Liquid Chromatography (HPLC) to a purity of >95%. The activated cysteine residue is introduced using Boc-Cys(NPys)-OH (Bachem product no. A-2825) as a building block. Fmoc-Dpr(Boc-Aoa)-OH((N-α-Fmoc-N-β-(N-t.-Boc-aminooxyacetyl)-L-diaminopropionic acid; Novabiochem product no. 04-12-1185) is used to introduce the N-β-aminooxyacetyl L-diaminopropionyl residue. Quality control (QC) of the purified peptide is done by amino acid analysis, electrospray mass spectroscopy (ESMS) and analytical reversed phase HPLC.

(ii) Synthesis of the Delivery Carrier Comprising Modules (a), (b) and (c) and the Linker (Method A):

To prepare module (a), recombinant Ricin toxin B subunit [(Ricin B; SEQ ID NO: 115), obtained from Vector Laboratories, Inc., catalog no. L-1290] and supplied as a 1 mg/mL solution in 10 mM aqueous sodium phosphate, 0.15 M NaCl, pH 7.5, containing 0.08% sodium azide and 50 mM 2-mercaptoethanol is supplemented with fresh 50 mM 2-mercaptoethanol and incubated for 1 h at room temperature (RT) to ensure that the Cys residue at position 4 from the C-terminus is completely in the fully reduced form. The solution is then desalted and buffer exchanged against degassed 10 mM sodium phosphate buffer, 150 mM NaCl, pH 7.4 (PBS) using a Vivaspin 20 protein concentrator (10 kDa MWCO, Sartorius Stedim Biotech product no. VS2001). Initially 20 mL of Ricin B solution is concentrated to a volume of 1 mL and the concentrated solution is washed with 6×15 mL of PBS at 4° C. A solution containing Ricin B (16 mg, 500 nmol) in PBS (16 mL) is reacted for 18 h at RT under nitrogen with 6.0 mg (1500 nmol) of the linkage molecule containing modules (b) and (c) from Example 1(i) above. Following a brief centrifugation the desired carrier [modules (a)+(b)+(c)] is then purified in 3 aliquots by preparative gel filtration [Size Exclusion Chromatography (SEC)] using a HiLoad 16/60 Superdex 75 prep grade column (GE Healthcare, part no. 17-1068-01) eluted with PBS containing 15 mM lactose at a flow rate of 1 mL/min. Identification of the desired carrier peak (retention time of 68 min) is enabled by having calibrated the SEC column with Ricin B (retention time 78 min) and with the linker-peptide entity (retention time 82 min) from 21(i). Product containing fractions are pooled and concentrated to a volume of 1 mL using a Vivaspin 20 protein concentrator (10 kDa MWCO). The product (4.3 mg, 120 nmol) is analyzed by ESMS and by native gel electrophoresis and compared to Ricin B and the linker-peptide.

(iii) Preparation of Cargo Compound (d) [an siRNA]:

A double stranded RNA molecule comprised of two 21 mer strands, with a double stranded region of 19 nucleotides in length and 2 nucleotides overhanging at the 3′ end of each strand, and targeting glyceraldehyde 3-phosphate dehydrogenase (GAPDH), wherein the sense strand comprises CCAuCUUCCAGGAGCgAGAuu (SEQ ID NO: 194), wherein lowercase u or g represents a 2′-O-Me-modified nucleotide; and the antisense strand comprises UCUCGCUCCUGgAAGAuGGdTdG (SEQ ID NO: 195), wherein lowercase u or g represents a 2′-O-Me-modified nucleotide and wherein the antisense strand has a 5′-phosphate and deoxynucleotides at its 3′ end (dNdN), is synthesized such that the 5P-terminus of the sense strand is modified with 5′-(C6 aminolinker)-phosphate-(C6-SS—C6 spacer)-phosphate-Cy3. The Cy3 dye is for tracking purposes by fluorescence and the disulfide bond ensures that the cargo can finally be released within the reducing environment of the cell cytoplasm. The single strands are analyzed by ESMS and analytical HPLC for QC prior to annealing. The desalted lyophilized siRNA (100 nmol) is dissolved in sterile sodium tetraborate buffer pH 8.5 and reacted with 10 molar equivalents of the linker molecule SFB (succinimidyl 4-formylbenzoate, Thermo Scientific, catalogue no. 22419) dissolved in 10% by volume of DMSO for 3 h at RT. The siRNA bearing a benzaldehyde function is isolated by dialysis against 100 mM sodium phosphate, 150 mM NaCl, 2 μM EDTA, pH 7 using a Slide-A-Lyzer dialysis cassette with a molecular weight cut-off of 3.5 kDa, volume 0.5-3 mL (Pierce no. 66330). Two dialyses are performed for 2 h each at RT followed by a third dialysis overnight at 4° C. The final solution is concentrated to a final volume of approximately 1 mL using a small ultrafiltration cell. QC of the linker modified siRNA is done by ESMS and analytical HPLC. A small aliquot of the sample is analyzed for the presence of the aldehyde moiety by reaction with an excess of Cascade Blue hydrazide (Molecular Probes, catalog no. C-687) in buffer at pH 5, desalted by ethanol precipitation and analyzed by native anion-exchange HPLC on a MonoQ column (GE Healthcare) using multiwavelength detection (260 nm for the RNA, 399 nm for the Cascade Blue and 550 nm for the Cy3).

(iv) Coupling of the Cargo [Compound (d)] to the Delivery Carrier [Modules (a)+(b)+(c) and a Linker]:

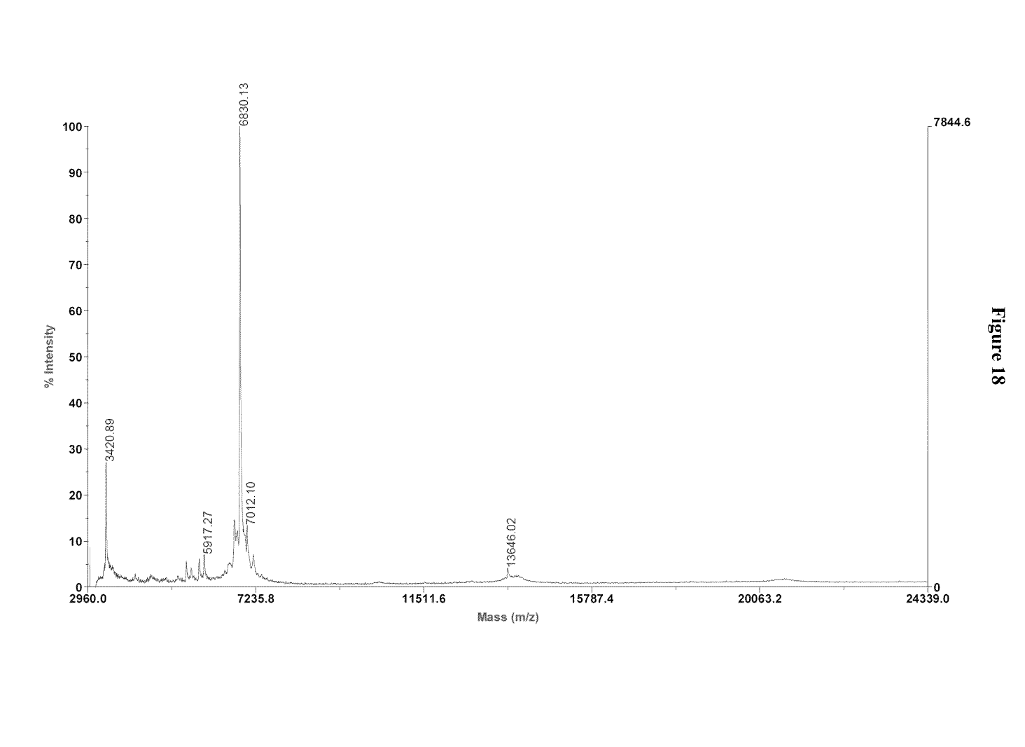

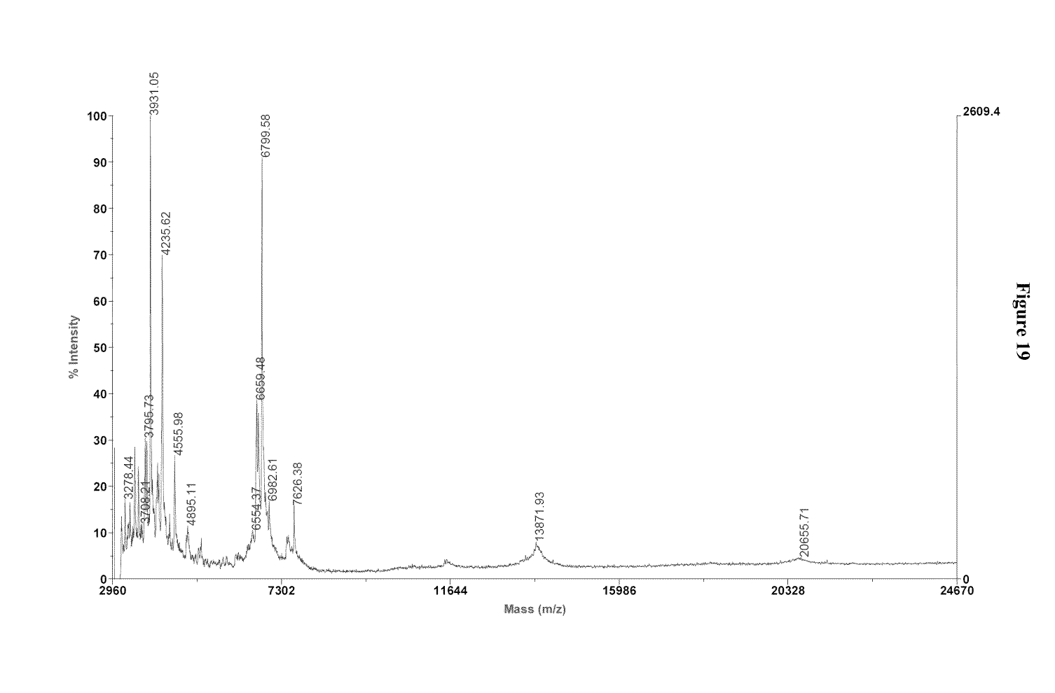

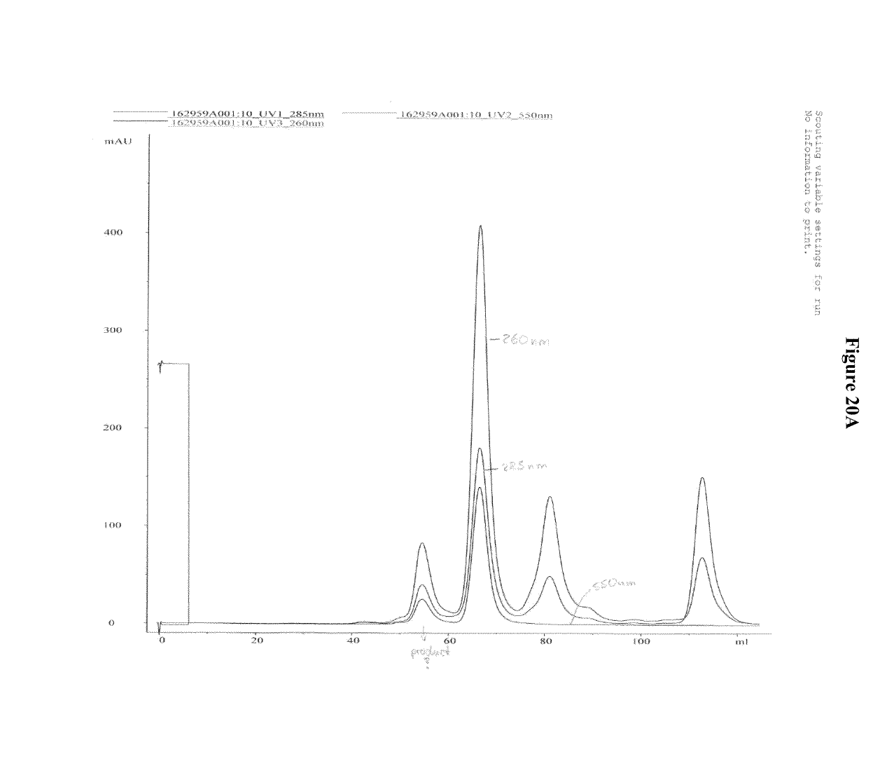

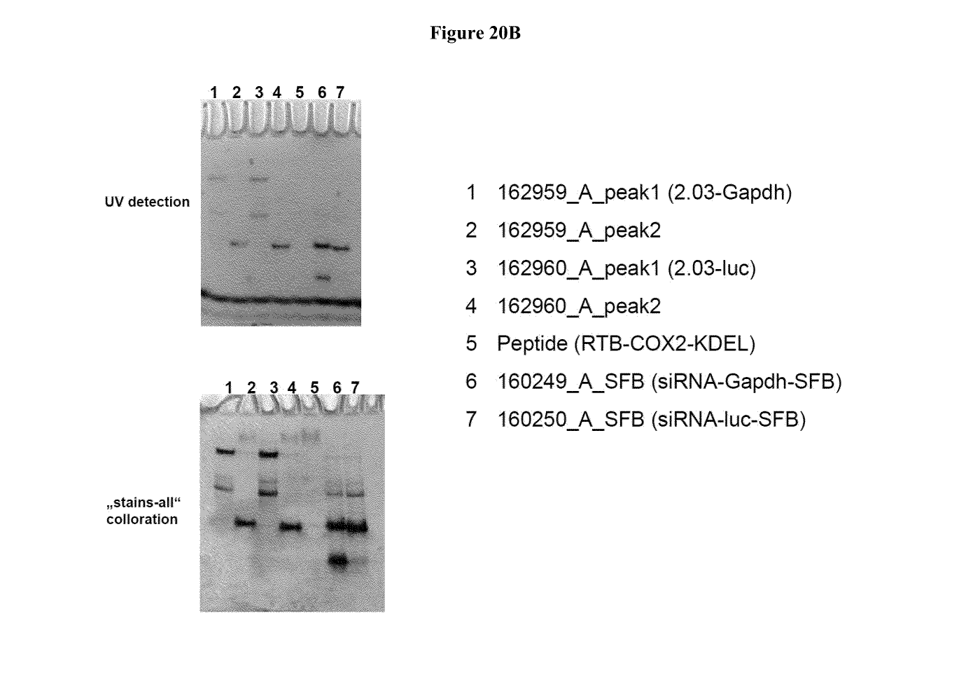

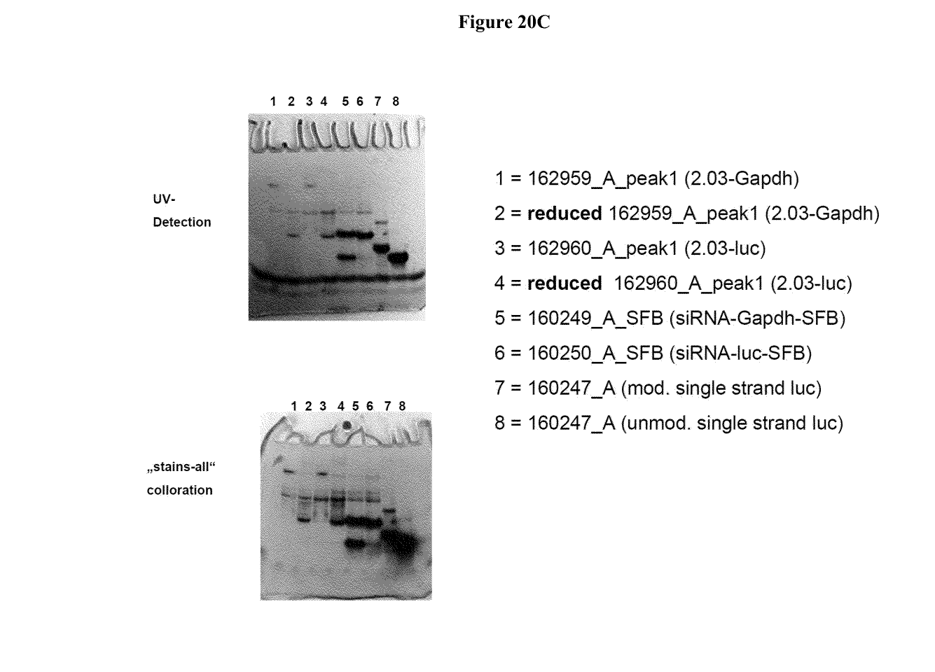

The carrier (50 nmol) from Example 1(ii) above in 500 μL of 100 mM phosphate buffer containing 100 mM aniline pH 7 is mixed with an approximately equimolar amount of the linker-siRNA component (cargo) from Example 1(iii) above and kept for 24 h at RT. The desired conjugate is purified by preparative SEC on a HiLoad 16/60 Superdex 75 prep grade column (GE Healthcare, part no. 17-1068-01) eluted at 1 mL/min with sterile PBS, pH 7.4, see FIG. 20A. The column effluent is monitored at 260, 285 and 550 nm. Calibration of the column is carried out prior to the preparative purification using the individual reaction components, and under these conditions the delivery carrier, RTB-COX2-KDEL elutes at 68 min (no absorbance at 550 nm) and the linker-siRNA elutes at 66 min (absorbance at all 3 wavelengths due to Cy3 presence) with an additional peak at 81 min due to excess antisense strand (no absorbance at 550 nm since no Cy3 attached). Peak 1 (FIG. 20A) elutes at 55 min and is the expected product, followed by peak 2 at 66 min (unreacted delivery carrier and unreacted linker-siRNA) followed by peak 3 at 81 min (excess antisense strand RNA) and finally peak 4 at 113 min (salt peak and aniline). Those fractions containing peak 1 the desired conjugate are combined and concentrated by ultrafiltration using a Vivaspin 20 concentrator (10 kDa MWCO) and the final concentrate is stored at 4° C. QC is performed by native gel electrophoresis (see FIG. 20B) and analytical SEC on Superdex 75 10/300 GL column (GE Healthcare, part no. 17-5174-01).

Since the DARE™ constructs comprise several components linked together covalently (in most cases by 2 disulfide bonds), and comprise polypeptides as well as a cargo molecule, it may be difficult to characterize them as single entities by molecular weight using standard mass spectroscopy (MS) techniques such as matrix assisted lased desorption ionization-time of flight (MALDI-TOF) mass spectroscopy or electrospray mass spectroscopy (ESMS). While characterization by PAGE or gel filtration certainly gives a general indication of their homogeneity, to be sure that the molecule isolated comprises all the expected component parts, it is preferred to incubate the DARE™ construct with a reducing agent such as dithiothreitol (DTT) or tris(2-carboxyethyl)phosphine (TCEP) to cleave all accessible disulfide bonds. This will generate 3 molecules, in the case of 2 disulfide (S—S) bonds, that can be separated by ion-pair reversed phase HPLC (UPLC) and characterized by ESMS. If necessary, the individual components may also be sequenced by special mass spectroscopy techniques such as MS-MS, however in most cases, it should suffice that the measured masses of the components match the expected (calculated) masses.

Thus, a small aliquot of the DARE 2.03-Gapdh product is treated with 15 mM dithiothreitol (DTT) in 0.5×PBS buffer containing 7.5 mM lactose during 20 min at room temperature to reduce the two accessible disulfide bonds to generate 3 reaction products (i.e., ricin B, linker-peptide construct plus adapter and HS—(CH₂)₆—OP(O₂)—O-Cy3-siRNA) that are analyzed by native PAGE and visualization by uv and also by “stains-all”, see FIG. 20C.

It will be apparent to one of skill in the art that the approach described within this Example may be used to attach other cargoes, e.g. a double stranded DNA, a single stranded miRNA antagonist (antagomir), an antisense oligonucleotide, and the like to a delivery carrier (i.e., [module (a)+module (b)+module (c)] of the present invention. It may be advantageous to attach the single stranded cargoes via their 3′-termini. The 3′-modified single strands are made by procedures that are standard to those skilled in the art.

A detailed drawing of conjugate DARE™-2.03, RTB-COX2-KDEL-siRNAas described in Example 1 is shown in FIGS. 2B and 7.

### Example (2)

### Synthesis of DARE™ Delivery Modules and Preparation of a Delivery siRNA Conjugate DARE™-R-CXpeg, (a DARE™ Delivery Vehicle Design 2.0)

(i) Synthesis of the Linkage Molecule Containing Modules (b) and (c):

The module (b)+module (c)+linker peptide H₂N—C(NPyS)(dPEG12)(DprAoa)(dPEG12) NASSSRSGLDDINPTVLLKERSTEL-OH [“module (b)+module (c)” functionalities are provided by a human COX2 peptide comprising an amino acid sequence comprising SEQ ID NO: 2; CXpeg] is synthesized commercially by standard solid-phase Fmoc peptide chemistry, deprotected in the standard fashion and purified by reversed phase HPLC to a purity of >95%. The activated cysteine residue is introduced using Boc-Cys(NPys)-OH (Bachem product no. A-2825) as a building block. Fmoc-Dpr(Boc-Aoa)-OH (Novabiochem product no. 04-12-1185) is used to introduce the N-β-aminooxyacetyl L-diaminopropionyl residue. dPEG12 is introduced using Fmoc-dPEG₁₂-acid (Quanta BioDesign, product no. 10283). QC of the purified peptide is done by amino acid analysis, ESMS and analytical reversed phase HPLC.

(ii) Synthesis of the Delivery Carrier [Linker Plus Modules (a), (b) and (c)]:

The synthesis of the delivery carrier from ricin B and the linker-peptide from Example 2(i) above is described in Example 1(ii) above. Briefly, a ricin B [module (a)] is prepared as described in Example 1(ii), then reacted overnight at RT under nitrogen with a PBS solution containing 1.1 mole equivalent of the [linker-module (c)-module (b)] product of Example 2(i). The delivery carrier [modules (a), (b), and (c) and the linker] is purified and analyzed as described above in Example 1(ii).

(iii) Preparation of the Cargo siRNA [Compound (d)]:

The cargo siRNA [compound (d)] is prepared as described in Example 1, section (iii) above.

(iv) Coupling of Compound (d) to the Carrier Module:

The components from Example 2(ii) and (iii) above are combined and the DARE™-R-CXpeg conjugate is isolated and analyzed as described in Example 1(iv) above.

### Example (3)

### Synthesis of a DARE™ Delivery Vehicle Design 3.1 with a Tet1 Peptide as Module (a) for Delivering an siRNA Cargo

This Example describes the preparation of a conjugate comprising a neuronal cell targeting peptide Tet1 [65, 66] as module (a). Tet1 protein targets neurons and has the same binding characteristics as tetanus toxin [65, 66].

(i.) Synthesis of a Tet1 Peptide Based Module (a):

A Tet1 peptide HLNILSTLWKYR-(flexible linker)-C (SEQ ID NO: 190), wherein the flexible linker is either GGG, SGSG, or SGSGSG, is synthesized by standard solid-phase Fmoc peptide chemistry, deprotected in the standard fashion and purified by reversed phase HPLC to a purity of >95%. QC of the purified peptide is done by amino acid analysis, ESMS and analytical reversed phase HPLC.

(ii.) Synthesis of the Linkage Molecule Containing Modules (b) and (c):

The [module (b)+module (c)+linker] peptide H₂N—C(NPyS)(dPEG12)(DprAoa)(dPEG12) NASSSRSGLDDINPTVLLKAKDEL-OH [the peptide comprising “module (b)+module (c)” comprises an amino acid sequence comprising SEQ ID NO: 3] is synthesized by standard solid-phase Fmoc peptide chemistry, deprotected in the standard fashion and purified by reversed phase HPLC to a purity of >95%. The activated cysteine residue is introduced using Boc-Cys(NPys)-OH (Bachem product no. A-2825) as a building block. Fmoc-Dpr(Boc-Aoa)-OH (Novabiochem product no. 04-12-1185) is used to introduce the N-β-aminooxyacetyl L-diaminopropionyl residue. dPEG12 is introduced using Fmoc-dPEG₁₂-acid (Quanta BioDesign, product no. 10283). QC of the purified peptide is done by amino acid analysis, ESMS and analytical reversed phase HPLC.

(iii.) Synthesis of the Delivery Carrier Comprising Modules (a), (b) and (c) and the Linker:

A solution containing module (a) from Example 3(i) above in 100 mM sodium phosphate, 150 mM NaCl, 2 mM EDTA, pH 7.5 is reacted overnight at RT under nitrogen with a solution containing 1.1 mole equivalents of the linkage molecule containing modules (b) and (c) from Example 3(ii) above in the same buffer. The desired carrier is then purified by preparative gel filtration (SEC) using a HiLoad 16/60 Superdex 75 prep grade column (GE Healthcare, part no. 17-1068-01) eluted with 100 mM sodium dihydrogen phosphate buffer, 100 mM NaCl, 2 μM EDTA, pH 5.0 at a flow rate of 1 mL/min. Identification of the desired carrier peak is enabled by having calibrated the SEC column with the 2 individual starting materials. The product is analyzed by ESMS, native gel electrophoresis and analytical reversed phase HPLC.

(iv.) Preparation of the Cargo siRNA [Compound (d)]:

A Tuschl-style siRNA targeting GAPDH is synthesized, purified and analyzed as described in Example 1(iii) with the 5′-terminus of the sense strand modified with 5′-(C6 aminolinker)-phosphate-(C6-SS—C6 spacer)-phosphate-Cy3.

(v.) Coupling Compound (d) to the Delivery Carrier:

The delivery carrier from Example 3(iii) above is mixed with an approximately equimolar amount of the linker-siRNA component (cargo) from Example 3(iv) above and kept for several hours at RT. The desired conjugate is purified by preparative SEC on a HiLoad 16/60 Superdex 75 prep grade column (GE Healthcare, part no. 17-1068-01) eluted at 1 mL/min with sterile PBS, pH 7.4. The column effluent is monitored at 260 nm and 550 nm. Calibration of the column is carried out prior to the preparative purification using the individual reaction components. Those fractions containing the conjugate are combined and concentrated by ultrafiltration (Vivaspin device) and the final concentrate is stored at 4° C. QC is performed by native gel electrophoresis and analytical SEC on a Superdex 75 10/300 GL column (GE Healthcare, part no. 17-5174-01). Additionally, a small aliquot of the product is treated with DTT to reduce the two accessible disulfide bonds to generate 3 reaction products (i.e., module (a), linker-peptide construct plus linker and HS—(CH₂)₆₋₀P(O₂)—O-Cy3-siRNA) that are analyzed by ESMS, analytical SEC using a Superdex 75 10/300 GL column eluted with PBS, and by analytical reversed phase HPLC.

### Example (4)

### Synthesis of a DARE™ Delivery Vehicle Design 3.2 with a Single Chain Antibody as Module (a) and an siRNA Cargo

(i) Synthesis of Module (a):

An anti-EGFR single chain antibody (SEQ ID NO: 196) is synthesized with an additional cysteine at the C-terminus using solid-phase Fmoc chemistry, deprotected in the standard fashion and purified by reversed phase HPLC to a purity of >95%. QC of the purified peptide is performed using amino acid analysis, ESMS and analytical reversed phase HPLC.

(ii) Synthesis of the Linkage Molecule Containing Modules (b) and (c):

The [module (b)+module (c)+linker] peptide N-acetyl-C(NPyS)(dPEG12)(DprAoa) (dPEG12)NASSSRSGLDDINPTVLLKAKDEL-OH [the peptide comprising “module (b)+module (c)” comprises an amino acid sequence comprising SEQ ID NO: 3] is synthesized by standard solid-phase Fmoc peptide chemistry, deprotected in the standard fashion and purified by reversed phase HPLC to a purity of >95%. The activated cysteine residue is introduced using Boc-Cys(NPys)-OH (Bachem product no. A-2825) as a building block. Fmoc-Dpr(Boc-Aoa)-OH (N-α-Fmoc-N-β-(N-t.-Boc-aminooxyacetyl)-L-diaminopropionic acid; Novabiochem product no. 04-12-1185) is used to introduce the N-β-aminooxyacetyl L-diaminopropionyl residue. dPEG12 is introduced using Fmoc-dPEG₁₂-acid (Quanta BioDesign, product no. 10283). QC of the purified peptide is done by amino acid analysis, ESMS and analytical reversed phase HPLC.

(iii) Synthesis of the Delivery Carrier Comprising Modules (a), (b), (c) and the Linker:

A solution containing module (a) from Example 4(i) above in 100 mM sodium phosphate, 150 mM NaCl, 2 mM EDTA, pH 7.5 is reacted overnight at RT under nitrogen with a solution containing 1.1 mole equivalents of the linkage molecule containing modules (b) and (c) from Example 4(ii) above in the same buffer and containing enough N,N-dimethylformamide (DMF) to ensure solubility of both components. The desired carrier is then purified by preparative gel filtration (SEC) using a HiLoad 16/60 Superdex 75 prep grade column (GE Healthcare, part no. 17-1068-01) eluted with 100 mM citrate buffer, 2 μM EDTA, pH 6.0 at a flow rate of 1 mL/min. Identification of the desired carrier peak is enabled by having calibrated the SEC column with the two individual starting materials. The product is analyzed by ESMS, native gel electrophoresis and analytical reversed phase HPLC.

(iv) Preparation of the Cargo siRNA [Compound (d)]:

A Tuschl-style siRNA targeting GAPDH is synthesized, purified, and analyzed as in Example 1(iii) with the 5′-terminus of the sense strand modified with 5′-(C6 aminolinker)-phosphate-(C6-SS—C6 spacer)-phosphate-Cy3.

(v) Coupling Compound (d) to the Delivery Carrier:

The carrier from Example 4(iii) above is mixed with an approximately equimolar amount of the linker-siRNA (cargo) from Example 4(iv) above and kept overnight at RT. The desired conjugate is purified and analyzed as described in Example 3(v) above. A small aliquot of the product is treated with DTT to reduce the two accessible disulfide bonds to generate 3 reaction products, viz. module (a), linker-peptide construct plus linker and HS—(CH₂)₆₋₀P(O₂)—O-Cy3-siRNA that are analyzed by ESMS, analytical SEC using a Superdex 75 10/300 GL column eluted with PBS, and by analytical reversed phase HPLC.

### Example (5)

### Synthesis of a DARE™ Delivery Vehicle Design 3.3a to Deliver a Non-Covalently Linked siRNA Cargo

(i) Construction of an Aldehyde Modified Transferrin as Module (a)

Human serum transferrin (SEQ ID NO: 191; Sigma, Invitrogen is reacted under mild conditions with sodium periodate to generate reactive aldehyde functionalities on the carbohydrate moieties using the published protocol of d'Alessandro et al. [67]. It has previously been shown that conjugation of peroxidase hydrazide to an aldehyde modified transferrin yields a bioconjugate that is fully recognizable by both anti-transferrin and anti-peroxidase antibodies [67].

(ii) Synthesis of a Linkage Molecule Comprising a Branched Peptide Moiety Containing Modules (b) and (c)

The PEG containing [module (b)+module (c)+linker] peptide construct 12-(aminooxy)dodecanoyl-(dPEG12)-bLys-(dPEG12)NASSSRSGLDDINPTVLLKAKDEL-OH [the peptide comprising “module (b)+module (c)” comprises an amino acid sequence comprising SEQ ID NO: 3], whereby the side chain amine of the branching lysine (bLys) residue in addition carries the sequence (dPEG12)Cys(NPys), is synthesized commercially by standard solid-phase Fmoc peptide chemistry, deprotected in the standard fashion and purified by reversed phase HPLC to a purity of >95%. The N-terminal 12-(aminooxy)-dodecanoyl moiety is introduced using 12-(Boc-aminooxy)-dodecanoic acid (Bachem, catalog no. A-4720). dPEG12 is introduced using Fmoc-dPEG₁₂-acid (Quanta BioDesign, product no. 10283). The branch point lysine residue is introduced using the Fmoc-Lys(ivDde)-OH (Merck Novabiochem, product no. 04-121193) building block. QC of the purified peptide is done by amino acid analysis, ESMS and analytical reversed phase HPLC.

(iii) Production of a Genetically Engineered DRBD Carrying an N-Terminal Cysteine:

A double stranded RNA binding domain (DRBD): FFMEELNTYRQKQGVVLKYQELPNS GPPHDRRFTFQVIIDGREFPEGEGRSKKEAKNAAAKLAVEILNKE (SEQ ID NO: 104) is produced genetically by recombinant engineering with an N-terminal Cys residue CFFMEELNTYRQKQGVVLKYQELPNSGPPHDRRFTFQVIIDGREFPEGEGRSKKEAKNA AAKLAVEILNKE (SEQ ID NO: 192), or alternatively, synthesized with an additional cysteine at the C-terminus FFMEELNTYRQKQGVVLKYQELPNSGPPHDRRFTFQVIIDG REFPEGEGRSKKEAKNAAAKLAVEILNKEC (SEQ ID NO: 193) using solid-phase Fmoc chemistry, deprotected in the standard fashion and purified by reversed phase HPLC to a purity of >95%. QC of the purified peptide is done by amino acid analysis, ESMS and analytical reversed phase HPLC.

(iv) Preparation of the siRNA Cargo Binding Construct Comprising the Targeting Module (a) Linked to the Sorting Modules [(b) and (c)] and the DRBD Adapter:

The aldehyde modified transferrin from Example 5(i) above is first reacted with 2 mole equivalents of the aminooxy bearing linkage molecule containing modules (b) and (c) from Example 5(ii) above in degassed 100 mM citrate buffer at pH 6 and kept overnight at 4° C. The desired intermediate is purified by preparative SEC on a HiLoad 16/60 Superdex 75 prep grade column (GE Healthcare, part no. 17-1068-01) eluted at 1 mL/min with sterile PBS, pH 7.4. This intermediate is then conjugated to the N-terminal cysteine containing DRBD from Example 5(iii) above via disulfide exchange with the Cys(NPys) residue in an overnight reaction in PBS at 4° C. The desired cargo binding modality is purified by preparative SEC on a HiLoad 16/60 Superdex 200 prep grade column (GE Healthcare, part no. 17-1069-01) eluted at 1 mL/min with sterile PBS, pH 7.4. Final QC analysis is performed by gel electrophoresis and ESMS, plus cleavage of the construct by DTT and analysis of the two components.

### Example (6)

### Synthesis of a DARE Delivery Vehicle Design 3.3b to Deliver a Non-Covalently Linked dsDNA Cargo

(i) Construction of an Aldehyde Modified Transferrin as Module (a)

Human serum transferrin (SEQ ID NO: 191; Sigma, Invitrogen) is reacted under mild conditions with sodium periodate to generate reactive aldehyde functionalities on the carbohydrate moieties using the published protocol of d'Alessandro et al. [67]. It has previously been shown that conjugation of peroxidase hydrazide to an aldehyde modified transferrin yields a bioconjugate that is fully recognizable by both anti-transferrin and anti-peroxidase antibodies [67].

(ii) Synthesis of a Linkage Molecule Comprising a Branched Peptide Moiety Containing Modules (b) and (c)

The PEG containing [module (b)+module (c)+linker] peptide construct 12-(aminooxy)dodecanoyl-(dPEG12)-bLys-(dPEG12)NASSSRSGLDDINPTVLLKAKDEL-OH [the peptide comprising “module (b)+module (c)” comprises an amino acid sequence comprising SEQ ID NO: 3], whereby the side chain amine of the branching lysine (bLys) residue in addition carries the sequence (dPEG12), is synthesized by standard solid-phase Fmoc peptide chemistry, deprotected in the standard fashion and purified by reversed phase HPLC to a purity of >95%. The N-terminal 12-(aminooxy)-dodecanoyl moiety is introduced using 12-(Boc-aminooxy)-dodecanoic acid (Bachem, catalog no. A-4720). dPEG12 is introduced using Fmoc-dPEG₁₂-acid (Quanta BioDesign, product no. 10283). The branch point lysine residue is introduced using the Fmoc-Lys(ivDde)-OH (Merck Novabiochem, product no. 04-121193) building block. QC of the purified peptide is done by amino acid analysis, ESMS and analytical reversed phase HPLC.

(iii) Preparation of the Arylhydrazine Containing Construct Comprising the Targeting Module (a) Linked to the Sorting Modules [(b) and (c)] and Linker:

The aldehyde modified transferrin from Example 6(i) above is first reacted with 2 mole equivalents of the aminooxy bearing linkage molecule containing modules (b) and (c) from Example 6(ii) above in degassed 100 mM citrate buffer at pH 6 and kept overnight at 4° C. The desired intermediate is purified by preparative SEC on a HiLoad 16/60 Superdex 75 prep grade column (GE Healthcare, part no. 17-1068-01) eluted at 1 mL/min with sterile PBS, pH 7.4. The primary amino group on the dPEG12 of this intermediate is then reacted with 4 mole equivalents of sulfosuccinimidyl 6-hydrazinonicotinate acetone hydrazone (sulfo-S-HyNic, sulfo-SANH, SoluLink product no. S-1011-010) in 100 mM HEPES, 150 mM NaCl pH 8.0 for 2 h at RT to introduce an arylhydrazine functionality protected as the acetone hydrazone. The activated construct is then desalted using a Vivaspin 2 polyethersulfone (PES) ultrafiltration spin column (molecular weight cut-off 5 kDa, Sartorius Stedim Biotech, part no. VS0211) and buffer exchanged into 100 mM citrate buffer pH 6.0.

(iv) Synthesis of an Aromatic Aldehyde Modified Adapter Molecule Derived from Human High-Mobility Group Protein HMGB2 (a DDBP) Carrying the SV40 NLS at its N-Terminus

SV40_{NLS}-HMGB2₁₈₆ is expressed in Escherichia coli using the published protocol of Sloots et al. [68], which is incorporated herein in its entirety by reference. The purified protein is reacted with 2 mole equivalents of MTFB (SoluLink product no. S-1035) in 100 mM citrate buffer pH 6.0 for 2 h at RT, which functionalizes a cysteine thiol with a 4-formylbenzamide moiety via a (PEG)₃ spacer. The desired activated construct is then desalted using a Vivaspin 2 polyethersulfone (PES) ultrafiltration spin column (molecular weight cut-off 5 kDa, Sartorius Stedim Biotech, part no. VS0211), using 100 mM citrate buffer pH 6.0 for washing.

(v) Synthesis of the dsDNA Cargo Binding Delivery Construct Comprising Module (a) Linked to the Sorting Modules (b) and (c) and the NLS Tagged DDBP Adapter.

The arylhydrazine modified targeting and sorting construct from Example 6(iii) above is mixed with an equimolar amount of the aldehyde modified adapter construct from Example 6(iv) above in 100 mM citrate buffer pH 6.0 and incubated overnight at RT to connect the two components via a stable bis-arylhydrazone bond. The desired dsDNA cargo binding delivery construct is purified by preparative SEC on a HiLoad 16/60 Superdex 200 prep grade column (GE Healthcare, part no. 17-1069-01) eluted at 1 mL/min with sterile PBS, pH 7.4. Final QC analysis is performed by gel electrophoresis.

(vi) Loading with dsDNA Cargo Binding Delivery Construct with a dsDNA Cargo.

The dsDNA cargo binding delivery construct from Example 6(v) above is mixed with a dsDNA (for instance a transcription factor decoy) in PBS pH 7.4 and incubated at RT for 30 min. The amount of dsDNA that can be bound will depend on the sequence length and is able to be determined by titration experiments and monitoring of the reaction by PAGE. The final DARE™ construct is purified on a preparative gel or by ion-exchange HPLC.

An optional biodegradable disulfide bond may also be included in the hydrazone linker fragment that covalently connects the targeting or sorting component to the DDBP adapter by using for example, S—SS-4FB (SoluLink product no. S-1037-010) as an aromatic aldehyde containing entity for modifying a primary amine

### Example (7)

### Use of a Targeted Delivery Carrier-Cargo Conjugate of the Invention to Elicit siRNA-Induced Silencing in Cultured Mammalian Cells

(i) Fluorescent Labeling of Protein Modules

In order to monitor the intracellular trafficking of module (a) alone, and module (a) with modules (b) and (c) by microscopy, the peptide or protein modules (a) can be labeled with a fluorescent dye. By way of example, ricin B is labeled with Cy3 Maleimide Monoreactive dye (GE Healthcare, PA23031) according to the manufacturer's protocol. Briefly, 1 mg/mL of full length ricin B-subunit (Vector Laboratories) is dialyzed against PBS supplemented with 1 mM EDTA. The terminal sulfhydryl group on the ricin B is made available by reduction with 100× molar excess of TCEP. The vial is flushed with nitrogen gas and closed. Sample is mixed thoroughly and incubated for 10 min at RT. An aliquot of Cy3 maleimide monofunctional dye, sufficient for the labeling of 1 mg of protein is dissolved in anhydrous dimethylformamide. The vial is flushed with nitrogen gas and closed. The sample is mixed thoroughly, incubated for 2 h at RT and mixed every 30 min. The reaction is left at 4° C. overnight. Separation of ricin B from the free dye is done by multistep dialysis against PBS. Absorbance of the sample at 552 nm and 280 nm is read in a spectrophotometer and the final dye/protein or dye/peptide ratio is calculated.

(ii) Preparation of a Dye Labeled-Module (a)+Module (b) Construct

Ricin B subunit [SEQ ID NO: 115; module (a)] is labeled with Cy3 NHS ester and then linked through a disulfide bond to a module (b) comprising a KDEL peptide (SEQ ID NO: 25) with a free C-terminus. Briefly, 0.5 mL of 1 mg/mL full length ricin B subunit (Vector Laboratories) in PBS containing 50 mM 2-mercaptoethanol (2-ME) is desalted and then buffer exchanged against sterile 100 mM sodium tetraborate buffer, pH 8.5 containing 5 mM lactose using a Vivaspin 2 polyethersulfone (PES) ultrafiltration spin column (molecular weight cut-off of 5 kDa, Sartorius Stedim Biotech, part no. VS0211) and then stirred in air to dimerize it, to prevent the thiol from potentially reacting with the Cy3 NHS ester in the subsequent reaction. The ricin B dimer is then fluorescently labeled by reaction with 4 molar equivalents (relative to ricin B monomer) of Cy3 NHS ester (GE Healthcare, catalog no. PA13101) dissolved in 25 μL of pure DMSO for 3 h at 10° C. The solution is then desalted on a Vivaspin 2 PES 5 kDa molecular weight cut-off spin column and transferred into PBS containing 5 mM lactose and 1 mM EDTA at pH 7. The Cy3-labeled ricin B dimer is reduced with fresh 50 mM 2-ME and incubated for 1 h at RT. The Cy3-labeled ricin B is recovered using a Vivaspin 2 PES, 5 kDa molecular weight cut-off spin column and buffer exchanged into degassed PBS containing 5 mM lactose and 1 mM EDTA, pH 7 and then reacted overnight at 10° C. under an argon atmosphere with 1.1 mole equivalents of the module (b) peptide, H₂N-Cys(NPys)-(SG)-3-KDEL-OH, prepared by standard solid-phase Fmoc peptide chemistry. The dye-labeled module (a)+module (b) construct is purified by gel electrophoresis.

(iii.) Monitoring Intracellular Sorting of DARE™ Modules in Cultured Cells

The following modules and conjugates are monitored:
Ricin B [module (a)], fluorescently labeled with Cy3 as described under Example 7(i) above
Ricin B [module (a)], including a C-terminally attached KDEL sequence [SEQ ID NO: 25; module (b)], prepared and fluorescently labeled with Cy3 as described under Example 7(ii) above
Ricin B [module (a)], including modules (b) and (c) as described in Example 1(i) and (ii), fluorescently labeled with Cy3 as described under Example 7(ii) above
Ricin B [module (a)], including modules (b) and (c), conjugated to an siRNA molecule as described in Example 1, wherein the siRNA is
   Specific and targeting GAPDH, or
   Non-specific, comprising a firefly luciferase fLuc:

| (SEQ ID NO: 197) | |
|---|---|
| sense: | 5′-CUUACgCUGAGuACUUCGAuu-3′, |
| and | |
| (SEQ ID NO: 198) | |
| antisense: | 5′-UCGAAGUACUCAgCGUAAgdTdG-3′, |

wherein the lowercase u or g represents a 2′-O-Me-modified nucleotide, and wherein the antisense strand has a 5′-phosphate and two deoxynucleotides at its 3′ end (dTdT).

HeLa (human), U2-OS (human) and NIH-3T3 (murine) cells are each grown on collagen coated 384-well plates suitable for microscopy (Aurora Biotechnologies) using Dulbecco's Modified Eagle Media (DMEM) supplemented with 4 mM glutamine (Invitrogen) and 10% fetal bovine serum (Invitrogen) under standard conditions. In order to monitor internalization and intracellular transport of DARE™ modules and conjugates, cells are treated with a range of 1-100 ng/mL of fluorescently labeled module/conjugate for 30 mM on ice, followed by 2-3 washing steps with cold medium, before warming up to 37° C. for different time periods ranging from 30 min to several hours (e.g. 0.5, 1, 2, 4, 6, 8, 16) and up to several days (e.g., 1, 2, 3, 4, 5, 6, 7). Alternatively, cells are incubated with the same amount of module/conjugate at 37° C. for the indicated time periods without a preceding binding and washing step on ice. At the indicated time points, cells are washed five (5) times with PBS, and fixed with 4% paraformaldehyde for 45 min. The cell membranes are permeabilized by incubation with 0.1-0.2% Triton X-100, and 0.01 to 0.02% Saponin in PBS for up to 30 min at RT. Non-specific binding sites are blocked by incubation with 10% fetal calf serum (Invitrogen) in PBS for 30 min. This step can optionally be combined with the permeabilization. The permeabilized cells are incubated with primary antibodies as listed below. Antibody incubations are performed in blocking buffer at 4° C. for up to 16 h. The cells are then washed with PBS and incubated with the appropriate fluorescently labeled (preferably with FITC or Alexa 488) standard secondary antibodies directed to the primary antibody at RT for 2 h, and then washed with PBS. Intracellular sorting of the modules/conjugates is determined by co-staining of the cells for intracellular compartments:
Endosomes:

Early and recycling endosomal compartments are identified through co-internalization of fluorescently labeled transferrin (Invitrogen, Alexa-633 conjugate, Catalog No. T-23362) at 10-100 μg/mL using the same experimental conditions as described for the modules and conjugates.

Late endosomal compartments are identified through co-internalization of fluorescently labeled LDL particles (LDL-DiI, bti inc. Stoughton Mass., USA) at 5-20 μg/mL, using the same experimental conditions as described for the modules and conjugates.
Lysosomes:

Lysosomes are identified by antibody staining using a rat monoclonal antibody (1D4B; ABCAM, Cambridge UK) to murine LAMP1 (lysosomal-associated membrane protein 1) at 0.1-0.5 μg/mL. Human LAMP 1 can be detected by staining using a rabbit polyclonal antibody at 1:500 (Abcam, ab24170).
Trans-Golgi-Network:

The trans-Golgi-network (TGN) is identified by antibody staining using a mouse monoclonal antibody (2F7.1; ABCAM, Cambridge UK) to TGN46 (trans golgi network protein of 46 kDa) at a dilution of 1:100 to 1:500.
Golgi Apparatus:

The Golgi Apparatus are identified by antibody staining using an antibody to mannosidase II (ab12277; ABCAM, Cambridge UK) at a dilution of 1:100 to 1:1000 in mouse cells. In human cells, the Golgi Apparatus can be detected by staining using a mouse monoclonal antibody against Golgin-97 (Invitrogen A-21270) at approximately 1 pg/mL.
Endoplasmic Reticulum (ER):

The ER is identified by antibody staining using a chicken polyclonal antibody to Calreticulin (ABCAM, Cambridge UK, ab14234) at a dilution of 1:500. Alternatively, ER exit sites can be stained by using a rabbit polyclonal antibody against Derlin-1 (Sigma, D4443) at a dilution of 1:200.
Caveolae:

Caveolae are identified by antibody staining using a rabbit monoclonal antibody to Caveolin-1 (New England Biolabs, D46G3) at a dilution of 1:500. Alternatively, caveolar internalization can be visualized by co-internalization with fluorescently labeled AMF (alias GPI, GeneID: 100008744). AMF labelling is done with a Fluorescein-EX labelling kit (Invitrogen). Cells are incubated with labelled AMF at 50 μg/mL [69, 70].
Cytoplasm:

Delivery of the siRNA [compound (d)] to the cytoplasm is followed by microscopy via the fluorescent dye attached to the 5′-end of the sense strand of the siRNA. Preferably the fluorescent dye is Cy3 or Cy5.

Images are acquired using an automated microscope (ImageExpress, Molecular Devices) or an LSM510 confocal microscope (Zeiss), and co-localization between the modules/conjugates and different cellular organelles/compartments is determined by automated image analysis (Cellenger, Definiens).

Alternatively, a multiparametric approach is used to detect colocalization of the conjugate and/or the modules and/or compound (d) of the invention and involves three different analysis techniques. In addition to the basic qualitative approach to identifying colocalization, two statistical methods are employed to quantitate colocalization using a Definiens Enterprise image analysis software.

For qualitative analysis of colocalization, captured channels are pseudo-colored using an appropriate color look-up table provided with the image analysis software, to convert greyscale into color, where x shade of grey equals y color. The Definiens system, for example, can convert a greyscale image into red, green, blue, yellow, violet or turquoise. Thus, if the pixels are co-stained with red and green, then yellow colored pixels indicate colocalization.

Quantitative statistical analyses using intensity correlation coefficient-based techniques are also performed, using two approaches, the Manders' coefficient, which is a modified version of the Pearson's coefficient, and L1's approach. Prior to calculation of coefficients, background is first excluded using a fluorescence intensity threshold, thereby identifying regions of interest. This background threshold is set manually for each assay. The Manders' coefficients, m₁ and m₂, are then calculated for all remaining pixels in each image:

m₁=Σᵢ^{S1i,coloc}/Σᵢ^{S1i}

m₂=Σᵢ^{S2i,coloc}/Σᵢ^{S2i}

Where, S1i,coloc is the sum of the intensities of channel 1 that colocalise with channel 2 and S1i is the sum of the intensities in channel 1. Similarly, S2i,coloc is the sum of the intensities of channel 2 that colocalise with channel 1 and S2i is the sum of the intensities in channel 2. When calculated, a Manders' coefficient of 1 indicates complete colocalisation and a coefficient of 0 indicates complete exclusion.

In contrast, Li's approach assumes that for two sets of random staining intensities of N number of pixels, the sum of the product of their differences will tend towards zero:

Σ_{N}(Aᵢ−a)(Bᵢ−b)˜0

Where a or b is the mean intensity of the distribution with N number of values of Aᵢ or Bᵢ, the intensity of each individual pixel. Intensity counts for each pixel in each image are therefore normalized to give a value between 0 and 1 and are plotted on a graph against the product of (Aᵢ−a)(Bᵢ−b) for each pixel, which varies between minus 1 and plus 1. In these graphs, pixels to the right of x=0 indicate colocalization, while pixels to the left of x=0 indicate complete exclusion.

A positive result using all three methods described above provides a very good assessment of colocalization [71-74].

(iv.) Testing for Degradation of the Delivery Carrier Modules

Cells are treated with a series of titrations of the modules/conjugates described in Example 7(iii) above, for different time periods ranging from 1 to 7 days. At the indicated times (1 h, 8 h, 1, 2, 3, 4, 5, 6, or 7 days), cells are lysed, and equal amounts of total protein are separated by SDS-PAGE. Degradation of the delivery carrier modules is monitored by western blotting and probing with an antibody directed against ricin B (obtained from ABCAM, Cambridge, UK, ab48415, used at a dilution of 1:100 to 1:1000).

(v.) Functional Testing of DARE™ Delivery

Cells are treated with a series of titrations of the modules/conjugates described in Example 7(iii) above, for different time periods ranging from 1 to 7 days. For comparison, cells are transfected with equimolar amounts of the targeting siRNA and the non-targeting control using commercially available transfection reagents, e.g. Dharmafect (ThermoFisher) or RNAiMax (Invitrogen). After the indicated time periods (1, 2, 3, 4, 5, 6, or 7 days), cells are lysed and tested for silencing of the target gene by quantitative RT-PCR (qRT-PCR), which is performed on a SDS7900 Thermocycler (Applied Biosystems) with gene specific validated TaqMan probes (Applied Biosystems), or gene specific primers and the SyBr-Green method, according to the manufacturers' recommendations. Gene expression is normalized to a housekeeping gene (e.g. 18S ribosomal RNA, RPL13A, or a specifically selected set of housekeeping genes if necessary [75]).

(vi.) Testing for Interferon Response Caused by DARE™ Delivery

Activation of the interferon pathway is monitored by determining expression levels of OAS1, OAS2, STAT1, IFNB1, and IFIT2 in treated cells compared to untreated cells by qRT-PCR as described above in Example 7(v.). Primer sequences of use to detect an interferon response by qRT-PCR of OAS1, OAS2, STAT1, IFN-beta and IFIT2 include commercially available Human TaqMan probes: OAS1 (Hs00973637_m1), OAS2 (Hs00942643_m1), STAT1 (Hs01014002_m1), IFN-beta (Hs00277188_s1), IFIT1 (Hs01911452_s1), and IFIT2 (Hs00533665_m1), and Mouse TaqMan probes: OAS1 (Mm00449297_m1), OAS2 (Mm00460961_m1), STAT1 (Mm00439518_m1), IFN-beta (Mm00439552_s1), IFIT1 (Mm00515153_m1), and IFIT2 (Mm00492606_m1) (Applied Biosystems/LifeTechnologies, Inc.).

While this Example illustrates the preparation, use and characterization of a ricin B [module (a)] targeted conjugate of the invention, the teachings of this Example are applicable to any conjugate of the invention. One of ordinary skill in the art will know how to modify the teachings of the Example accordingly and without undue experimentation.

### Example (8)

### Synthesis of a DARE™ Delivery Construct with Target siRNA as Compound (d) but Without the Cell Targeting/Uptake Module (a)

(i) Synthesis of the Linkage Molecule Comprising Modules (b) and (c)

The [module (b)+module (c)+linker] peptide H₂N—(SG)₃-C—(SG)₃-NASSSRSGLDDINPTV LLKAKDEL-OH [“module (b)+module (c)” comprise SEQ ID NO: 3] is synthesized by standard solid-phase Fmoc peptide chemistry, deprotected in the standard fashion and purified by reversed phase HPLC to a purity of >95%. QC of the peptide is done by amino acid analysis, mass spectroscopy and analytical reversed phase HPLC. Activation of the free thiol of the purified peptide is done by reaction in pyridine with 1.5 mole equivalents of 2,2′-dithiobis(5-nitropyridine) (DTNP, Sigma-Aldrich product no. 158194) to give H₂N—(SG)₃-C(pNpys)-(SG)₃-NASSSRSGLDDINPTVLLKAKDEL-OH, which is purified by preparative reversed phase HPLC to >95% purity and analyzed as noted above.

(ii) Preparation of the Cargo siRNA [Compound (d)]

A Tuschl-style siRNA targeting GAPDH is synthesized, purified and analyzed as described in Example 1(iii) except that the 5′-terminus of the sense strand is modified with a 5′-(C₆—SS—C₆)-phosphate-Cy3 entity.

(iii) Preparation of the [Module (b)+Module (c)+Module (d)] Construct in which (d) is siRNA

The cargo siRNA from Example 8(ii) above is treated with 100 mM DTT in PBS containing 1 mM EDTA for 1 h at 37° C. to cleave the disulfide bond. The free thiol containing siRNA is then desalted on a Vivaspin 2 polyethersulfone 3 kDa molecular weight cut-off ultrafiltration spin column (Sartorius Stedim Biotech, part no. VS0292) using degassed PBS containing 1 mM EDTA pH 7 as eluent. The thiol-siRNA is subsequently reacted overnight under argon with 1.1 mole equivalents of the linkage molecule containing modules (b) and (c) from Example 8(i) above in PBS containing 1 mM EDTA pH 7. The desired module (b)+module (c)+module (d) construct is purified by reversed phase HPLC. The product is analyzed by ESMS, native gel electrophoresis and analytical HPLC. Further analysis is done using DTT cleavage to obtain two fragments, the molecule comprising modules (b) and (c), and the HS—(CH₂)₆—OP(O₂)—O-Cy3-siRNA, that can each be separately identified by MS.

Although this specific example describes the use of a COX2 peptide as the ERAD targeting module (c), an AKDEL peptide (SEQ ID NO: 26) as the ER translocation module (b), and the attachment of the siRNA through a disulfide bond to a cysteine residue, one of skill in the art is able to envision and make conjugates comprising other peptide(s) and using a different attachment and/or a different configuration without undue experimentation that are also embodiments of the present invention. For example, (SG)₃ (SEQ ID NO: 98) can be replaced by dPEG12 and the siRNA may be attached via an oxime bond using the aminooxy group on a DprAoa residue (i.e., instead of the cysteine in this Example).

### Example (9)

### Pharmacodynamics of a DARE™ Delivery Conjugate

To evaluate the in vivo activity of a DARE™ delivery conjugate of the present invention, the pharmacodynamics are tested after systemic application. A DARE™ delivery construct of the present invention is administered intravenously in mice via the tail vein (or alternatively intraperitoneally). Bio-distribution is determined in two different mouse models. In one model, an endogenously expressed gene (GAPDH) is targeted; in the second model, an exogenously introduced reporter transgene (firefly luciferase, fLuc) is targeted. A non-silencing siRNA conjugate and a non-targeting [i.e., lacking module (a)] conjugate are also prepared as controls.

(i) Synthesis of the Conjugates

All conjugates are prepared as described in Examples 1 and 6, and the following siRNA sequences are preferably used:

### GAPDH:

sense: SEQ ID NO: 194 and
antisense: SEQ ID NO: 195
fLuc:
sense: SEQ ID NO: 197, and
antisense: SEQ ID NO: 198,
Non-silencing control (targeting NP number 2, a nucleoprotein of influenza virus):
sense: 5′-GGAuCUUAUUUCUuCGGAGuu-3′ (SEQ ID NO: 199), and
antisense: 5′-CUCCGAAGAAAuAAGAuCCdTdT-3′ (SEQ ID NO: 200), wherein “u” and “g” represents 2′-O-Me-modified nucleotides and all antisense strands have a 5′-phosphate.

(ii) In Vivo Testing

The GAPDH targeting conjugate is tested for GAPDH specific knockdown in Balb/c mice [available from Jackson Laboratories (www.jax.org), Charles River (www.criver.com), Taconic (www.taconic.com), or Harlan (www.harlan.com)], while luciferase knockdown is evaluated in a mouse strain that is transgenic for firefly luciferase (Promega pGL3) and expresses high levels of the enzyme in virtually all tissues [76]. Gender matched mice that are 6-10 weeks of age are used.

A dose escalation of the DARE™ delivery construct is performed, for example using a range of 100 to 2000 nmol/kg. The DARE™ delivery construct dose is then injected in a volume of 100-300 μL PBS (or other physiological buffer). As described within this Example, the dose at which the highest knock down of fLuc is achieved, while avoiding lethality, is determined. This dose is preferably used subsequently for all other systemic applications.

Each experiment consists of the following groups with n=10 mice/group.
1. DARE™ delivery construct with target siRNA (directed to either GAPDH or luciferase and corresponding to the in vivo model used) as compound (d), prepared as described in Example 1
2. DARE™ delivery construct with non-target siRNA as compound (d), prepared as described in Example 1
3. DARE™ delivery construct with target siRNA as compound (d) but without a cell targeting/uptake module (a), prepared as described in Example 8 above.

Mice are euthanized at 24-72 h post DARE™ delivery construct dose injection and tissues of interest (e.g. brain, lung, heart, liver, kidney, spleen, muscle, ovaries, uterus, mammary glands, pancreas, lymph nodes, bone, and any other tissue of interest) are sampled and analyzed as described below.

### Luciferase Measurements:

For luciferase protein measurement, tissues are homogenized using a tissue lyser/mixer mill (Qiagen), metal beads and luciferase cell culture lysis reagent (e.g. Promega PR-E1531), and then centrifuged for 5 min at maximum speed (13,000g) in a table top centrifuge before the supernatant is transferred to a new reaction tube. The supernatant is either stored at −80° C. or used immediately to measure luciferase protein levels in a luminometer, using a luciferase assay system (e.g. Promega) according to the manufacturer's instructions.

### RNA Isolation:

Tissue samples are stored in RNAlater (Qiagen) for subsequent qRT-PCR and 5′-RACE analysis or frozen in liquid nitrogen for subsequent luciferase and tissue protein (to normalize for luciferase activity per mg protein) quantification. After euthanasia, the tissues/organs of interest are removed and immediately frozen in liquid nitrogen. RNA is isolated from the tissue samples with the RNeasy kit (Qiagen) according to the manufacturer's instructions and RNA quality is determined with an Agilent 2100 Bioanalyzer using the RNA 6000 Nano kit (Agilent) according to the manufacturers' instructions.

### 5′ RACE-PCR:

5′RACE is performed to detect RNAi specific RNA degradation products. The detection is performed by a modified GeneRacer PCR (Invitrogen, Calsbad, Calif.) as described before [77-79]. Briefly, a 44 mer RNA-oligo, which is a pre-designed kit component (GeneRacer™ RNA Oligo) is ligated to 5′-uncapped, degraded RNA before reverse transcription. Following this, a PCR is performed with a primer set consisting of a gene-specific primer 3′ of the siRNA recognition site and a complementary primer binding to the 44 mer RNA-Oligo sequence:

For GAPDH (human and mouse) the sequences
are as follows:
GAPDH siRNA target sequence:
(SEQ ID NO: 201)
5′-GGTCATCCATGACAACTTT-3′;
GeneRacer 5′ Primer:
(SEQ ID NO: 202)
5′-CGACTGGAGCACGAGGACACTGA-3′;
GAPDH 3′ Primer:
(SEQ ID NO: 203)
5′-ACGCCTGCTTCACCACCTTCTTGATGTC-3′;
GeneRacer 5′ Nested Primer:
(SEQ ID NO: 204)
5′-GGACACTGACATGGACTGAAGGAGTA-3′;
and
GAPDH 3′ Nested Primer:
SEQ ID NO: 205)
5′-AGGCCATGCCAGTGAGCTTCCCGTTCAG-3′.

Agarose gel analysis and sequencing of the amplified DNA is then used to identify the resulting DNA fragment as an RNAi specific degradation product of the gene of interest. In case of low abundant degradation products, a nested PCR is carried out after the primary PCR.

### RT-qPCR:

RT-qPCR is performed on a SDS7900 Thermocycler (Applied Biosystems) with gene specific validated TaqMan probes (Applied Biosystems) according to the manufacturer's recommendations. Gene expression is normalized to a pool of housekeeping genes (e.g. 18S rRNA, RPLPO, Hmbs, Ppib, and/or Pgkl) selected for gene expression analysis in mouse tissue to normalize for natural expression variation in vivo [75].

### GAPDH ELISA and Western Blots:

GAPDH protein expression is determined with a standard GAPDH specific ELISA assay (e.g. from BIOO Scientific). Tissue is lysed by the addition of RIPA (Radio-immunoprecipitation assay; Sigma Aldrich) buffer and total protein concentration is measured by BCA assay (Bicinchoninic acid; Perbio) prior to analysis by ELISA according to the manufacturer's instructions or by western blot analysis according to standard procedures.

### RNA In Situ Hybridization:

In situ RNA detection is performed according to established procedures including Proteinase K digestion and acetic anhydride pre-treatment. The tissue sample is fixed in 4% PFA for 24-30 h after extraction before soaking in 30% sucrose for 24-30 h. It is then cooled to −70° C. in isopentane and 5 μm thick sections are cut in a cryostat-microtome. A GAPDH-specific digoxygenin labeled probe is prepared from a GAPDH cDNA containing plasmid with and SP6 or T7 RNA polymerase with the DIG RNA labeling Kit (Roche Applied Science) according to the manufacturer's recommendations and as described earlier [80]. The probe is incubated on the tissue sections in a humidified chamber at 65° C. overnight. The DIG labeled probe is detected with a sheep anti-DIG antibody conjugated to alkaline phosphatase (AP; Roche). The sections are then developed by the addition of BM purple (Roche) or another AP substrate.

### Immunohistochemistry and Histology:

For distribution analysis of the fluorescently (e.g.Cy3) labeled DARE™ delivery construct and analysis of target protein expression by immunohistochemistry, tissues are fixed in 4% paraformaldehyde, 0.05% glutaraldehyde in PBS for 24 h and then soaked in 30% sucrose for 36 h. The tissues are then frozen at −80° C. for storage, and 7 μm sections are cut at −20° C. and placed on slides. Microscopy analysis is performed as described above in Example 7.

For antibody staining and histology, tissue is fixed overnight in 10% buffered formalin before paraffin embedding and sectioning on a microtome. GAPDH protein expression is detected using a GAPDH specific antibody (rabbit mAB 14C10, Cell Signaling, or similar). Antigen detection is performed according to the manufacturer's recommendations following microwave assisted antigen retrieval using citrate buffer. Detection of primary antibody is done with an anti-rabbit HRP or fluorophore labeled secondary antibody (Abcam) before microscopy analysis using standard protocols or, in the case of a fluorophore labeled secondary antibody, as described above in Example 7.

### Example (10)

### Pharmacokinetics of a DARE™ Delivery Construct

To determine the knock down effect over time, a blood clotting factor, Factor VII (FVII) is targeted in the liver using a DARE™ delivery construct according to the present invention. Published siRNA sequences against FVII [81] or previously in vitro optimized siRNAs against FVII are used as compound (d) in a DARE™ delivery construct and made as described in the Examples above. The optimal knock down dose of the resulting DARE™-FVII conjugate is determined in liver in experiments as described in Example 9. The DARE™-FVII conjugate is then tested in vivo at this optimal knock down dose.

All procedures are done in normal C57BL/6 or Balb/c mice (gender and age matched, 6-10 weeks of age, obtained from Charles River). The optimal knock-down dose of DARE™-FVII is administered intravenously to mice via tail vein injection. Control mice are injected via the tail vein with the same DARE™ delivery construct as DARE™-FVII except that the control DARE™ construct comprises a non-targeting control siRNA as compound (d) instead of the siRNA against FVII. Blood samples are taken retro-orbitally from the DARE™-FVII treated and control treated mice repeatedly, on a twice weekly basis, until 40 days post injection and serum levels of FVII protein are measured using an activity-based chromogenic assay (Biophen FVII; Aniara, Mason, Ohio) [81] to determine the length of time that FVII protein levels remain knocked-down below that of the control mice. Based upon the length of time it takes for the circulating FVII protein levels of the DARE™-FVII treated mice to reach the circulating FVII protein levels of the control treated mice (i.e., baseline FVII levels), repeated administration times can be calculated. For example, if the circulating FVII protein levels of the DARE™-FVII treated mice reach the baseline FVII levels at 30 days post injection, repeated injections of the DARE™-FVII dose will be made every 30 days and retro-orbital blood samples will obtained and analyzed twice weekly. If the circulating FVII levels decrease and increase in similar fashion after a second and third injection of DARE™-FVII, then this indicates that there is no strong immune response against DARE™-FVII.

### Example (11)

### Testing for Immunostimulatory Effects of a DARE™ Delivery Conjugate

SiRNA molecules have been shown to stimulate the immune system via interaction with the toll-like receptors TLR3, TLR7 and/or TLR8 [82]. The immune responses to TLR7/8 can be overcome or at least minimized by chemically modifying the siRNAs Immunological responses resulting from such interactions can be examined in human PBMCs (peripheral blood monocytes) as described [83, 84]. Briefly, buffy coats are obtained from the blood of human donors. PBMCs are purified from the buffy coats by Ficoll density centrifugation. The purified PBMCs are then seeded in 96 well plates at 2×10⁵ cells/well or a different previously optimized density. The cells are then incubated at 37° C. with the siRNA, which is complexed with a transfection reagent or coupled to other molecules enabling transfection, i.e. a DARE™ delivery conjugate (final concentration: up to 1 μM). At different time points (e.g. 4 h and 24 h post transfection), supernatant is removed and the TNFα and/or IFNα concentration is determined via ELISA and compared to untreated PBMCs. The ELISAs are performed using commercially available ELISA kits [TNFα Elisa Jumbo Kit, #1M 11121, Beckman Coulter; and Human IFNα ELISA (multi species), #3169016, Thermo Fisher Scientific].

TLR7 and TLR8 mediate an inflammatory response caused by activation of the innate immune response [82]. TLR8, which is an important mediator of nonspecific siRNA immune effects in human cells, is not fully functional in mice [83]. Consequently, effects related to TLR8 are not relevant to mouse studies. To evaluate possible TLR8 mediated effects, human PBMCs can be used as described above. These cells will produce TNFα, even if the oligonucleotide only stimulates TLR8 but not TLR7 [83]. Thus, incubating human PBMCs with the DARE™ construct (at up to 1 μM final concentration), followed by a TNFα ELISA will be sufficient to evaluate a TLR7 and a TLR8 mediated response.

In addition, immune responses could also result from the DARE™ module(s) that transports the siRNA. Regarding an immediate immune response, the same assays as described above for siRNA will be sufficient for their characterization. If a delayed immune response occurs, e.g. mediated by antibodies, it will be detected when the DARE™ conjugate is administered a second time after approximately 30 days in an animal experiment, and the knock down effect is significantly reduced (see Examples 9 and 10 re: in vivo knock down).

In addition to the above and to further ensure that the effects observed with a DARE™ delivery conjugate of the present invention are sequence specifically mediated by the DARE™-delivery conjugate siRNA [compound (d)] and not by target-unrelated reactions to the siRNA or the DARE™ modules or delivery conjugate, knockout (k.o.) mice of the relevant TLR3 and TLR7 receptors can be used (TLR3 k.o. mice: B6; 129S1-Tlr3^{tmlFlv}/J, http://jaxmice.jax.org/strain/005217.html and TLR7 k.o. mice: B6.129S1-Tlr7^{tmlFlv}/J, http://jaxmice.jax.org/strain/008380.html, both from Jackson Laboratories). Specific effects via the DARE™ delivery conjugate siRNA will be the same in wt mice and in k.o. mice for the TLRs of the same strain (C57BL/6 is the wt strain corresponding with the above k.o. strains, available from Jackson Laboratories www.jax.org, Charles River www.criver.com, Taconic www.taconic.com, or Harlan www.harlan.com). For all experiments, gender and age matched mice (6-10 weeks of age) are used. These animal experiments are helpful to differentiate between the effects attributed to the siRNA [compound (d)] and the effects that may be produced by the immune system or an anti-angiogenic effect.

Specifically, GAPDH (or another endogenous gene) is targeted with an siRNA [compound (d)] of a DARE™ delivery construct according to the present invention. K.o. mice or cells as described above are used to evaluate the effects mediated by TLRs. The mice or cell experiments are analyzed as described in the Examples above by qRT-PCR, 5′RACE, Western blot and/or an enzymatic assay (e.g. KDalert™ GAPDH Assay Kit from Invitrogen/Life Technologies) for GAPDH expression.

Different versions of the modular DARE™ conjugate of the present invention are prepared according to Example 1 and delivered systemically via tail vein injection into mice. Each experimental group consists of 10 animals. Each experiment includes the following groups:
1. DARE™ delivery construct with a non-target siRNA as compound (d)
2. DARE™ delivery construct with a target siRNA as compound (d)
3. DARE™ delivery construct without an siRNA [i.e., lacking compound (d)]
4. Naked target siRNA (i.e., compound (d) only).

The optimal DARE™ dose as determined above in Example 9 is used here to determine whether any of the observed effects of the DARE™ constructs of the present invention are mediated by TLRs. The mice (or cells) are maintained for 2-60 days, depending on when the siRNA mediated effects are expected to occur. If GAPDH is used, the mice are analyzed after 48 h, at which time, the mice are euthanized and tissue samples are collected from the major organs (i.e., liver, spleen, kidney, brain, heart). When a tumor model is used, the mice are observed for up to 60 days. At each time point, animals are euthanized and tissues of interest as well as tumor samples are collected. The collected tissues and tumor samples are processed and analyzed for knock down expression of the targeted gene (i.e. GAPDH) by qRT-PCR, 5′RACE and western blot analysis as described above in Example 9.

### Example (12)

### Analysis of DARE™ Delivery Conjugate Toxicity

The potential toxicity of a DARE™ delivery conjugate of the present invention is assessed by measuring serum levels of liver enzymes and cytokines repeatedly up to 48 h post injection. A DARE™ construct with a non-targeting siRNA as compound (d) and a DARE™ construct without an siRNA [i.e., lacking a compound (d)] will be compared against PBS injection. The DARE™ delivery constructs are injected via tail vein injections as described in Example 9 above. Blood samples are collected retro-orbitally from the mice repeatedly up to 48 h post-injection and serum is obtained. Serum levels of the mouse cytokines TNF-alpha and IL-6 are measured by sandwich ELISA with reagents according to the manufacturer's instructions (R&D Systems, Minneapolis, Minn.). Serum levels of mouse IFN-alpha are measured by using a sandwich ELISA kit according to the manufacturer's instructions (PBL Biomedical, Piscataway, N.J.). Serum levels of alanine aminotransferase (ALT) and aspartate aminotransferase (AST) are measured by using automated systems at a veterinary diagnostic laboratory. If any statistically significant increases in liver enzymes and/or cytokines are detected, then further investigations should be conducted to determine the full toxicological impact of the conjugate.

### Example (13)

### Preparation and Administration of a DARE™ Delivery Conjugate having a VEGF-Specific siRNA as Compound (d) In Vivo: Xenograft Model for Oncology

To demonstrate efficacy of a DARE™ delivery construct of the present invention in a tumor model, a well-established xenograft tumor model is used to study the knockdown of tumor relevant targets.

In this Example, the expression of VEGF (Vascular endothelial growth factor) is knocked down and the effect of this knockdown on tumor vascularization and growth is evaluated [85-92]. The experiments are carried out in two independent tumor models in gender and age matched (6-10 weeks) immunoincompetent mice (preferably athymic nude mice, Harlan-Winkelmann). PC-3 prostate adenocarcinoma cells (ATCC CRL 1435) are injected subcutaneously at 3×10⁶ in 0.1 mL of serum-free F-12K medium (Invitrogen) into the dorsal flank region of the mouse. After the tumors are clearly established and reach a volume of 50-100 mm³, the control siRNA and DARE™ delivery conjugate formulations are delivered systemically by tail vein injections or intratumorally in independent experiments.

The following constructs and conjugates are prepared following the teachings of Examples 1 and 5. Each experiment consists of 5 groups, with n=14 mice/group:
1. DARE™ delivery construct without siRNA [i.e., lacking compound (d)]
2. Naked VEGF Target siRNA Sequence comprising a sense strand comprising 5′-GGAGUACCCUGAUGAGAUCdTdT-3′ (SEQ ID NO: 206), and an antisense strand comprising 5′-GAUCUCAUCAGGGUACUCCdTdT-3′ (SEQ ID NO: 207).
3. Naked non-target (Luciferase) siRNA comprising a sense strand comprising SEQ ID NO: 197, and an antisense strand comprising SEQ ID NO: 198.
4. DARE™ delivery construct with a compound (d) comprising a VEGF siRNA comprising a sense strand comprising SEQ ID NO: 206, and an antisense strand comprising SEQ ID NO: 207.
5. DARE™ delivery construct with a compound (d) comprising a non-target siRNA comprising a sense strand comprising SEQ ID NO: 199, and an antisense strand comprising SEQ ID NO: 200.

siRNA sequences targeting VEGF are selected based on published sequences [92, 93]. Doses range from 100 to 2000 nmol/kg in 100 μL for systemic delivery and 0.05 to 5 nmol in 25 μL for local intratumoral delivery.

To minimize an immunogenic effect on vascularization as previously reported [94], chemically modified siRNA sequences including selective introduction of 2′-O-Me nucleosides into the antisense strand are used [95, 96]. Non-targeting siRNA controls are optimized for this system to match the immunostimulatory effect of the VEGF targeted siRNA [97]. To assess immunostimulatory capacity of the siRNAs, a panel of cytokines and cytokine triggered mRNA is measured from mouse serum and target tissue, respectively. The immuno markers include, but are not limited to, interferon-α (IFNα), IL-6, IFNγ, tumor necrosis factor-α (TNFα), IL-12 and interferon induced tetratricopeptide repeat protein 1 (IFIT-1 or p56) mRNA [98, 99]. Mouse serum is analyzed for cytokines using commercially available ELISA assays, following standard procedures at 1-48 h after siRNA injections. IFIT mRNA levels are assessed at 1-48 h after siRNA injections by RT-qPCR with commercially available TaqMan probes as described in Example 9.

In the first part of this study, 6 animals are used per group for molecular analyses Animals are euthanized 2 days post treatment. In the second part of this study, 8 animals are used per group to analyze tumor growth/remission and vascularization. Animals are observed for up to 3 months or until moribund. Molecular analyses are carried out as follows or as described in Example 9:

### RNA Isolation:

After euthanasia, tumors are removed and immediately frozen in liquid nitrogen. RNA is isolated from tumor tissue with the RNeasy kit (Qiagen) according to the manufacturer's manual and RNA quality is determined with an Agilent 2100 Bioanalyzer using the RNA 6000 Nano kit (Agilent) according to the manufacturer's instructions.

### 5′ RACE-PCR:

5′ RACE-PCR is performed on individual tumor samples as described above in Example (9) using VEGF specific 5′ and 3′ primers and nested primers.

### RT-qPCR:

RT-qPCR is performed on individual tumor samples using an SDS7900 Thermocycler (Applied Biosystems) with gene specific validated VEGF TaqMan probes (Hs00900055 ml, Applied Biosystems) according to the manufacturer's recommendations. Gene expression is normalized to a pool of housekeeping genes (e.g. 18S rRNA, RPLPO, Hmbs, Ppib, and/or Pgkl) selected for gene expression analysis in PC3 tumors to normalize for natural expression variation in vivo as previously described [75].

### VEGF ELISA:

VEGF protein expression is determined for individual tumor samples using a standard ELISA assay. Tumor tissue is lysed by the addition of RIPA buffer (Sigma Aldrich) and concentration measured by BCA assay (Perbio) according to the manufacturer's instructions. VEGF ELISA is performed with a commercial Quantikine human VEGF Immunoassay kit (R&D systems) according to the manufacturer's instructions.

### RNA In Situ Hybridization:

For RNA in situ hybridization, tumors are removed and immediately frozen in liquid nitrogen. Ten (10) μm Microtome sections are placed on microscope slides and fixed with 4% PFA. Detection is performed according to established procedures including Proteinase K digestion and acetic anhydride pre-treatment. A VEGF-specific DIG labeled probe is prepared from a VEGF cDNA containing plasmid with the DIG RNA labeling Kit (Roche Applied Science) according to the manufacturer's recommendations as published before [80]). The probe is incubated on the tissue sections in a humidified chamber at 65° C. overnight. The DIG labeled probe is detected with a sheep anti-DIG antibody conjugated to alkaline phosphatase (AP; Roche). The sections are then developed by the addition of BM purple (Roche) or another AP substrate.

### Efficacy Studies:

To determine the efficacy of the DARE™ delivery conjugate comprising a VEGF siRNA as compound (d), tumor size and the extent of tumor vascularization following treatment are determined. All control groups are similarly monitored for comparison.

### Tumor Growth/Remission:

Tumor size is measured every other day with a calliper, beginning on the date of treatment.

### Tumor Vascularization:

After termination of the experiment to assess tumor growth in response to DARE™-siRNA treatment, the extent of tumor vascularization is assessed as described before [86, 100]. Tumors are fixed in 10% buffered formalin before they are paraffin embedded and cut on a Microtome to obtain 5-15 μm sections. Hematoxylin and eosin (H&E) staining and immunohistochemistry for CD31 (to visualize blood vessels) expression is performed. Tumor tissue sections are pretreated with 0.1% trypsin for 10-15 min at 37° C. before incubation with rat anti-mouse CD31 (mAb MEC13.3, PharMingen, San Diego, Calif.) at a 1:500 dilution overnight at 4° C. Immunoreactivities are preferably visualized with the avidin-biotin complex technique using Vectastain Elite ABC kit (Vector Laboratories, Burlingame, Calif.) with diaminobenzidine as chromogen, or alternatively, by immunofluorescence. For comparison of vascularization, intratumoral CD31 positive vessels are counted per field of view.

### Example (14)

### Preparation and Administration of a DARE™ Delivery Conjugate having a Bcl-xL Specific siRNA as Compound (d) In Vivo: Xenograft Model for Oncology

In this Example, the expression of the anti-apoptotic protein Bcl-xL is knocked down in a well established xenograft tumor model and its effect on tumor growth and apoptosis is determined [101, 102]. The experiments are carried out in gender and age matched, immuno-incompetent mice using PC-3 prostate adenocarcinoma cells (ATCC CRL 1435) as described above in Example 13.

The constructs and conjugates are prepared following the teachings of Examples 1 and 5. Each experiment consists of 5 groups, with n=14 mice/group:
1. DARE™ delivery construct without siRNA [i.e., lacking compound (d)]
2. Naked target Bc1-xL siRNA comprising a sense strand comprising 5′-GGUAUUGGUGAGUCGGAUCdTdT-3′(SEQ ID NO: 208), and an antisense strand comprising 5′-GAUCCGACUCACCAAUACCdTdT-3′ (SEQ ID NO: 209).
3. Naked non-target (Luciferase) siRNA comprising a sense strand comprising SEQ ID NO: 197, and an antisense strand comprising SEQ ID NO: 198.
4. DARE™ delivery construct with a compound (d) comprising target Bc1-xL siRNA comprising a sense strand comprising SEQ ID NO: 208, and an antisense strand comprising SEQ ID NO: 209.
5. DARE™ delivery construct with a compound (d) comprising a non-target siRNA comprising a sense strand comprising SEQ ID NO: 199, and an antisense strand comprising SEQ ID NO: 200.

Doses range from 100 to 2000 nmol/kg in 100 μL for systemic delivery and 0.05 to 5 nmol in 25 μL for local intratumoral delivery.

In the first part of this study, 6 animals are used per group for molecular knock-down analyses and animals are euthanized 2 days post treatment. In the second part of this study, 8 animals are used per group to analyze tumor growth/remission and apoptosis. Animals are observed at least twice weekly for up to 3 months or until moribund. Molecular analyses are carried out as follows or as described in Example 9 and Example 13.

### RNA Isolation:

After euthanasia, tumors are removed and immediately frozen in liquid nitrogen. RNA is isolated from tumor tissue with the RNeasy kit (Qiagen) according to the manufacturer's manual and RNA quality is determined with an Agilent 2100 Bioanalyzer using the RNA 6000 Nano kit (Agilent) according to the manufacturer's instructions.

### 5′ RACE-PCR:

5′ RACE-PCR is performed on individual tumor samples as described above in Example 9 using Bc1-xL specific 5′ and 3′ primers and nested primers.

### RT-qPCR:

RT-qPCR is performed on individual tumor samples using an SDS7900 Thermocycler (Applied Biosystems) with gene specific validated Bc1-xL TaqMan probes (Hs00236329_ml, Applied Biosystems) according to the manufacturer's recommendations. Gene expression is normalized to a pool of housekeeping genes (e.g. 18S rRNA, RPLPO, Hmbs, Ppib, and/or Pgkl) selected for gene expression analysis in PC-3 tumors to normalize for natural expression variation in vivo as previously described [75].

### Bc1-xL ELISA:

Bc1-xL protein expression is determined for individual tumor samples using a standard ELISA assay. Tumor tissue is lysed by the addition of RIPA buffer (Sigma-Aldrich) and concentration measured by BCA assay (Perbio) according to the manufacturer's instructions. Bc1-xL protein levels in the tumors are determined using a commercially available human Total Bc1-xL DuoSet ELISA kit (R&D Systems) according to the manufacturer's instructions.

### RNA In Situ Hybridization:

For RNA in situ hybridization, tumors are removed and immediately frozen in liquid nitrogen. Ten (10) μm Microtome sections are placed on microscope slides and fixed with 4% PFA. Detection is performed according to established procedures including Proteinase K digestion and acetic anhydride pre-treatment. A Bc1-xL-specific DIG labeled probe is prepared from a plasmid containing Bc1-xL cDNA. This is done with a DIG RNA labeling Kit (Roche Applied Science) according to the manufacturer's recommendations as previously described [80]. The probe is incubated on the tissue sections in a humidified chamber at 65° C. overnight. The DIG labeled probe is detected with a sheep anti-DIG antibody conjugated to alkaline phosphatase (AP: Roche). The sections are then developed by the addition of BM purple (Roche) or another AP substrate.

### Efficacy Studies:

To determine the efficacy of the DARE™ delivery conjugate comprising a Bc1-xL siRNA as compound (d), tumor size and the extent of tumor cell apoptosis following treatment are determined. All control groups are similarly monitored for comparison.

### Tumor Growth/Remission:

Tumor size is measured every other day with callipers, beginning on the date of treatment.

### Tumor Cell Apoptosis:

After termination of the experiment to assess tumor growth in response to DARE™-siRNA treatment, tumor cell apoptosis is analyzed using a TUNEL assay (Terminal deoxynucleotidyl transferase-mediated dUTP nick-end labelling) as previously described [102, 103]. For this purpose, tumors are immediately frozen after extraction. Sections of 4 um are cut with a cryostat and fixed in acetone before the TUNEL stain is performed. Total cell numbers are determined by DAPI (Invitrogen) nuclei staining and images of the sections are acquired by fluorescence microscopy. Fractions of apoptotic (TUNEL positive) cells are calculated by automated analysis with Definiens enterprise software (Definiens).

### Example (15)

### Administration of a DARE™ Delivery Conjugate to Deliver Compound (d) In Vivo: Syngeneic Model for Oncology

In addition to the xenograft models in Examples 13 and 14, the DARE™ delivery conjugate of the present invention is examined in a syngeneic tumor model to assess its activity and distribution in an immunocompetent mouse model with more natural vascularization compared to a xenograft model. For this purpose, FVB/N mice are inoculated with firefly luciferase expressing DB7 tumor cells. DB7 tumor cells were originally derived from FVB/NTg(MMTV-PyVmT Y315F/Y322F) mice and have been previously described [104]. To increase tumor take, the cells were passaged through FVB/N mice before implantation. For imaging purposes, DB7 cells were transduced with a retroviral vector [105] expressing a dual function reporter gene (L2G) comprised of firefly luciferase (fLuc) and green fluorescent protein (GFP) driven by a hybrid promoter consisting of the β-actin promoter and the cytomegalovirus enhancer (CAGS). Transduced cells were screened for fLuc expression with an IVIS 50 system (Caliper LifeSciences, Hopkinton, Mass.) and 25 positive clones selected and combined to obtain a population representative of the parental population (DB7luc+).

To study the tumor penetration and efficacy of DARE™-siRNA delivery conjugates of the present invention, gender and age matched mice (6-10 weeks of age) are injected with 2.5×10⁶ DB7luc+ cells subcutaneously. Tumors are allowed to establish for 2 weeks before the conjugates are injected.

The siRNA sequence for luciferase is optimized in vitro or an already described sequence [76] is used. siRNAs are controlled for immunostimulatory effects as described in Example 11.

siRNA and DARE™ construct formulations are prepared as described in Examples 1 and 5 are delivered systemically by tail vein injections or intratumorally in independent experiments. Each experiment consists of 5 groups with n=5 mice/group:
1. DARE™ delivery construct without siRNA[i.e., lacking compound (d)];
2. Naked fLuc siRNA comprising a sense strand comprising SEQ ID NO: 197, and an antisense strand comprising SEQ ID NO198;
3. Naked non-target siRNA comprising a sense strand comprising SEQ ID NO: 199, and an antisense strand comprising SEQ ID NO: 200;
4. DARE™ delivery construct with fLuc siRNA comprising a sense strand comprising SEQ ID NO197, and an antisense strand comprising SEQ ID NO: 198 as compound (d); and
5. DARE™ delivery construct with a non-target siRNA comprising a sense strand comprising SEQ ID NO: 199, and an antisense strand comprising SEQ ID NO: 200 as compound (d).

Doses used range from 100 to 2000 nmol/kg in 100 μL for systemic delivery and 0.05 to 5 nmol in 25 μL for local intratumoral delivery. Mice are euthanized at several time points post DARE™ injection (ranging from 1-7 days) and the tumors removed for molecular analysis as follows or as described above in Example 9. Tumors are stored in RNAlater (Qiagen) for subsequent analysis of fLuc mRNA levels by qRT-PCR and RNAi specific degradation of fLuc mRNA by 5′-RACE using fLuc specific 5′ and 3′ primers and nested primers. For quantification of luciferase and total tissue protein levels (to obtain the amount of luciferase per protein tissue), the tumors are frozen in liquid nitrogen. For luciferase enzyme activity measurement, the tumor is homogenized, using a tissue lyser/mixer mill (Qiagen), metal beads and luciferase cell culture lysis reagent (Promega PR-E1531), centrifuged for 5 min at maximum speed in a table top centrifuge (13,000g) before the supernatant is transferred to a new reaction tube. The supernatant is either stored at −80° C. or used immediately to measure luciferase in a luminometer, using a luciferase assay system (Promega) according to the manufacturer's instructions.

### Example (16)

### Demonstration of DARE™ Conjugate Delivery In Vivo: Local Delivery to the Central Nervous System (CNS)

Different versions of a modular DARE™ delivery conjugate of the present invention are delivered to the brain in a mouse model.

The following siRNA sequences are preferably used:

### GAPDH:

sense: SEQ ID NO: 194;
antisense: SEQ ID NO: 195;

### Non-Silencing Control:

sense: SEQ ID NO: 257, and
antisense: SEQ ID NO: 258.

The constructs and conjugates are prepared as described in Example 1. GAPDH specific knockdown is tested in Balb/c mice. Gender and age matched mice (6-10 weeks of age) are used. Single injections and long-term infusions are performed. Each experiment includes the following groups with n=10 animals/group:
1. DARE™ delivery construct without siRNA [i.e., lacking compound (d)]
2. Naked GAPDH siRNA comprising a sense strand comprising SEQ ID NO: 194, and an antisense strand comprising SEQ ID NO: 195;
3. Naked non-target siRNA comprising a sense strand comprising SEQ ID NO: 199, and an antisense strand comprising SEQ ID NO: 200;
4. DARE™ delivery construct with GAPDH siRNA comprising a sense strand comprising SEQ ID NO: 194, and an antisense strand comprising SEQ ID NO: 195 as compound (d); and
5. DARE™ delivery construct with a non-target siRNA comprising a sense strand comprising SEQ ID NO: 199, and an antisense strand comprising SEQ ID NO: 200 as compound (d).

For local delivery to the caudate putamen, single injections of 1 μL of DARE™ (total doses ranging from 0.05 to 5 nmol) in PBS are injected. Before the injection, animals are anaesthetized preferably by i.p. injection of 3.6% chloral hydrate (10 mL/kg) in H₂O, which is reapplied at half dose in the case where an animal begins to wake up. In preparation for the injection, the animal is then positioned in a stereotaxic apparatus (Axel Semrau, Sprockhoevel, Germany). After opening the skin by a scalpel incision, the skull is cleaned and opened with a fine drill (0.5 mm diameter) in preparation for the injection with a Hamilton syringe. Drilling and injections are performed according to the stereotaxic coordinates previously described [106, 107]. For injections into the caudate putamen, the coordinates for the tip of the syringe are (from bregma): Lateral −1.6 mm, Dorso-Ventral −3.8 mm, Anterior-Posterior −0.5 mm.

For long-term delivery, a DARE™ conjugate of the present invention is delivered via an osmotic pump (Alzet brain infusion kit) into the third ventricle at AP: −0.5 mm; ML: 0 mm, DV: −3 mm, relative to Bregma) as previously described [108, 109]. Briefly, the animals are prepared as above for single injections before a cannula ending at the appropriate coordinates is implanted and fixed to the skull. The osmotic pump is filled with a DARE™ conjugate of the present invention to achieve a delivery rate of 0.01 to 0.5 nmol per day in a daily volume of 5 μL for an infusion period of 2 weeks. The pump is implanted subcutaneously in the neck of the animals and connected to the cannula via silicone tubing.

Following the single injections, the animals are euthanized at 1-7 days post-injection. In the case of the infusions, the animals are euthanized immediately after the 2 weeks of infusion. The brain of each animal is immediately removed and processed for analysis of DARE™ distribution and efficacy as follows or as described above in Example 7 and Example 9. For RNA and protein analysis, the brains are dissected immediately following death of the animal and tissue is collected from different areas of interest and immediately frozen in liquid nitrogen. RNA is isolated with the Qiagen RNeasy Lipid tissue kit according to the manufacturer's manual. RT-PCR and 5′-RACE are performed as described in Example 9 above.

### Immunohistochemistry:

For distribution analysis of the Cy3 labeled DARE™ construct and analysis of protein expression by immunohistochemistry, the brain of each animal is fixed in 4% PFA, 0.05% glutaraldehyde in PBS for 24 h before being soaked in 30% sucrose for 36 h. The brain tissue is then frozen at −80° C. for storage, and 7 μm sections are cut at −20° C. and placed on slides for microscopy analysis. GAPDH protein expression is detected using a GAPDH specific antibody (rabbit mAB 14C10, Cell Signaling, or similar). Antigen detection is performed according to the manufacturer's recommendations following microwave assisted antigen retrieval using citrate buffer. Detection of the primary antibody is done with an anti-rabbit horseradish peroxidise (HRP- or fluorophore-labeled secondary antibody (Abcam) and then analyzed by microscopy using standard protocols or, in the case of a fluorophore labeled secondary antibody, as described above in Example 7.

### RNA In Situ Hybridization:

In situ RNA detection is performed according to established procedures including Proteinase K digestion and acetic anhydride pre-treatment. Brain tissue is fixed in 4% PFA for 24-30h after extraction before soaking in 30% sucrose for 24-30 h. It is then cooled to −70° C. in isopentane and 5 μm thick sections are cut in a cryostat-microtome. A target-specific digoxygenin labeled probe is prepared from a GAPDH cDNA containing plasmid with and SP6 or T7 RNA polymerase with the DIG RNA labeling Kit (Roche Applied Science) according to the manufacturer's recommendations and as described earlier [110]. The probe is incubated on the tissue sections in a humidified chamber at 65° C. overnight. The DIG labeled probe is detected with a sheep anti-DIG antibody conjugated to alkaline phosphatase (AP; Roche). The sections are then developed by the addition of BM purple (Roche) or another AP substrate.

While this Example illustrates the preparation, use and characterization of a specific, ricin B-[i.e., module (a)] targeted conjugate of the invention to deliver a GAPDH targeted siRNA as compound (d), the teachings of this Example are applicable to any conjugate of the invention. In particular, one of skill in the art may replace the GAPDH targeted siRNA with another siRNA directed against a target in which CNS gene expression knockdown is desired. In addition, one of skill in the art can replace the GAPDH targeted siRNA of the conjugate described in this Example with another compound (d) that is desired to be delivered to a cell in the CNS. As described above, modules (a), (b) and (c) can also be modified accordingly by one of skill in the art to suit the intended purpose and target cell within the CNS. These embodiments may be prepared without undue experimentation and are encompassed within the scope of the present invention.

### Example (17)

### Use of Chemical Inhibitors of the Retrograde Pathway to Monitor DARE™ Conjugate Delivery via Retrograde Transport

To monitor DARE™ conjugate delivery via retrograde transport, one can use chemical inhibitors or drugs that interfere in these pathways. These drugs have been commonly used in the literature and include brefeldin A (disrupts Golgi) and monensin (modulates transport to the Golgi, e.g. low concentrations increase ricin toxicity while higher concentrations protect against it) [111]. Thus, one can follow the DARE™ conjugates through the cell via co-stainings for the different organelles.

Retrograde pathway inhibitors are expected to prevent the transport from the endosome to the Golgi. If the inhibitor does indeed inhibit the transport of a conjugate of the present invention, indicated by a reduced RNAi effect and/or by confocal microscopy (i.e., wherein a fluorescently labeled DARE™ construct is no longer able to reach the ER), then this result indicates that the retrograde pathway is used by the DARE™ conjugate to deliver its compound (d) to the cytosol. Thus, if a DARE™ conjugate according to the present invention trafficks through the retrograde pathway to reach the ER, then pre-treatment of the cells with a retrograde pathway inhibitor before DARE™ conjugate addition should result in a reduction in fluorescently labeled DARE™ conjugates in the ER of the cells. Further, if inhibitor pre-treatment results in a reduced RNAi effect, then the DARE™ conjugate most likely uses the retrograde pathway to deliver its compound (d) (i.e., the siRNA cargo) to the cytosol.

Brefeldin A (BFA; Sigma-Aldrich, product no. B5936) is added to the cells with a final concentration of 5 μg/mL. This concentration results in rapid fusion of the Golgi with the ER within 30 min [111, 112]. However, a lower concentration of BFA of 0.5-1 μg/mL is sufficient in some cell lines to inhibit retrograde transport while enhancing cell survival for 1-3 days [111,112]. BFA also causes the fusion of early endosomes and the TGN.

Alternatively, nordihydroguaiaretic acid (NDGA; Sigma-Aldrich, product no. 74540), a lipoxygenase inhibitor, is added to the cells (in serum free medium) with a final concentration of 25 μM. This concentration results in rapid fusion of the Golgi with the ER within 30 min [113-115].

Alternatively, cyclofenil diphenol (CFD; Sigma-Aldrich, product no. C3490-10MG), a non-steroidal estrogen, is added to the cells with a final concentration of 25 μM. This concentration results in rapid fusion of the Golgi with the ER within 30 min.

Alternatively, Retro-1 or Retro-2 (Chembridge, www.chembridge.com) added to the cells with a final concentration of 25 μM. These latter two inhibitors do not cause fusion of cell organelles but specifically inhibit toxins (ricin, Shiga toxin, and the like) from being transported from the endosome to the TGN [new 116].

As a further alternative to the above inhibitors, Golgicide A (Sigma-Aldrich, product no. G0923-5MG, [117]) or other inhibitors of retrograde transport can be used.

The inhibitor of retrograde transport is added 30 min prior to the addition of the DARE™-siRNA construct. Knock down of the target mRNA and the target protein (e.g. GAPDH or luciferase) is evaluated after 6, 24 and 48 h using RT-qPCR and the appropriate protein assays, e.g. standard GAPDH enzyme activity assay or luciferase activity assay, as described in Example 9. Incubation with the inhibitor may be stopped by changing the medium before the incubation period is over if the inhibitor shows excessive cell toxicity; e.g. the inhibitor is removed after 6 h (or earlier) by changing the medium but the RT-qPCR and the protein assays are still performed after 24 and 48 h.

In addition or as an alternative to the RNAi experiments described above, retrograde transport can also be demonstrated via immunohistochemical analysis. NIH-3T3, HeLa or other appropriate cell lines are incubated with the DARE™-siRNA construct, which carries a fluorophore such as Cy3, for 15-60 min, followed by a medium change. At several time points thereafter (e.g. 30 min, 1, 2, 4, 6 and 24 h), the cells are fixed, stained with antibodies for different cell organelles and examined by confocal microscopy. During the incubation with the DARE™-siRNA, the inhibitor is added to half of the wells to demonstrate the use of the retrograde pathway for the transport of the DARE™-siRNA. For organelle markers, the following are used: Transferrin conjugated to a fluorophore to stain the early and recycling endosome (added to the cells when the DARE™-siRNA is added); LAMP1 antibody to stain lysosomes; Mannosidase II antibody to stain the Golgi Apparatus; Calreticulin, Calnexin (or Derlin-1) antibody to stain the ER; and nuclei can be stained with Hoechst dye (Invitrogen).

### Example (18)

### siRNAs Against Key Genes of the Retrograde Pathway

Knock down of key components of the retrograde pathway and ERAD via siRNA(s) that target these key components can also be used to track the pathway of conjugates of the invention. As an alternative to Example 17's use of chemical inhibitors of the retrograde pathway, key proteins for the retrograde transport of the DARE™-siRNA can also be knocked down with an siRNA. The analyses are identical to those described above in Example 17, i.e. reduced knock down by DARE™-siRNA and inhibited retrograde transport of the DARE™ siRNA. One to two days prior to the addition of DARE™-siRNA to the cells, the cells are transfected with an siRNA against one or several of the following genes: KDELR-1 (Accession number 10945), KDELR-2 (Accession number 11014), KDELR-3 (Accession number 11015), Sec61a1 (Accession number 29927), Derlin-1 (also referred to as DERL-1, Accession number 79139), PDIA2 (Accession number 64714), and ErolL (Accession number 30001), comprising one of the following siRNA sequences or an siRNA sequence as prepared by one of skill in the art:

| KDELR-1: | |
|---|---|
| (SEQ ID NO: 210) | |
| sense: | 5′-CUACCUCUAUAUCACCAAATT-3′, |
| (SEQ ID NO: 211) | |
| antisense: | 5′-UUUGGUGAUAUAGAGGUAGAA-3′, |
| KDELR-2: | |
| (SEQ ID NO: 212) | |
| sense: | 5′-AUAGGAGCAGGCAAGGUAGAT-3′, |
| (SEQ ID NO: 213) | |
| antisense: | 5′-CUACCUUGCCUGCUCCUAUTT-3′, |
| KDELR-3: | |
| (SEQ ID NO: 214) | |
| sense: | 5′-ACUGAUUCCAGAUAGAUAGAG-3′, |
| (SEQ ID NO: 215) | |
| antisense: | 5′-CUAUCUAUCUGGAAUCAGUTT-3′, |
| Sec61a: | |
| (SEQ ID NO: 216) | |
| sense: | 5′-GGAAUUUGCCUGCUAAUCATT-3′, |
| (SEQ ID NO: 217) | |
| antisense: | 5′-UGAUUAGCAGGCAAAUUCCAG-3′, |
| Derlin-1: | |
| (SEQ ID NO: 218) | |
| sense: | 5′-GCUUAGCAAUGGAUAUGCATT-3′, |
| (SEQ ID NO: 219) | |
| antisense: | 5′-UGCAUAUCCAUUGCUAAGCCA-3′, |
| PDIA2: | |
| (SEQ ID NO: 220) | |
| sense: | 5′-GUCGGAAGGUGAUUGAAUATT-3′, |
| (SEQ ID NO: 221) | |
| antisense: | 5′-UAUUCAAUCACCUUCCGACCT-3′, |
| Ero1L: | |
| (SEQ ID NO: 222) | |
| sense: | 5′-GGAAUGUCAUCUACGAAGATT-3′, |
| and | |
| (SEQ ID NO: 223) | |
| antisense: | 5′-UCUUCGUAGAUGACAUUCCAT-3′. |

### Example (19)

### DARE™ Conjugates Comprising at Least Two Compound (d) Molecules per Conjugate

This Example describes the preparation of a conjugate comprising 2 compounds (d), wherein the compounds (d) are two of the same target siRNA (see FIG. 14). One of skill in the art can appreciate that by increasing the number of compound (d) molecules conjugated to the conjugate of the present invention, one can increase the potency of the conjugate and thus, the delivery system of the present invention. In the case where the at least 2 compounds (d) are siRNAs, a positively charged molecule (i.e., spermine, spermidine or a positively charged peptide) may need to be added to the formulation, or may need to be used at a higher concentration in the formulation than required for the single siRNA-conjugate of the present invention, to compensate for the increased negative charge due to multiple siRNAs.

(i) Synthesis of the Linkage Molecule Comprising Modules (b) and (c):

The [module (b)+module (c)+2 linkers] peptide H₂N—C(NPys)-(SG)-3-(DprAoa)(dPEG12) (DprAoa)-(SG)₃-NASSSRSGLDDINPTVLLKAKDEL-OH [the peptide comprising “module (b)+module (c)” comprises an amino acid sequence comprising SEQ ID NO: 3] is synthesized commercially by standard solid-phase Fmoc peptide chemistry, deprotected in the standard fashion and purified by reversed phase HPLC to a purity of >95%. QC of the peptide is done by amino acid analysis, mass spectroscopy and analytical reversed phase HPLC. The activated cysteine residue is introduced using Boc-Cys(NPys)-OH (Bachem product no. A-2825) as a building block. Fmoc-Dpr(Boc-Aoa)-OH (Novabiochem product no. 04-12-1185) is used to introduce the N-β-aminooxyacetyl L-diaminopropionyl residue. dPEG12 is introduced using Fmoc-dPEG₁₂-acid (Quanta BioDesign, product no. 10283). QC of the purified peptide is done by ESMS and analytical reversed phase HPLC.

(ii) Synthesis of the Delivery Carrier Comprising Modules (a), (b) and (c) and 2 Linkers:

To prepare module (a), recombinant ricin toxin B subunit (SEQ ID NO: 115; Vector Laboratories, Inc., catalog no. L-1290) and supplied as a 1 mg/mL solution in 10 mM aqueous sodium phosphate, 0.15 M NaCl, pH 7.5, containing 0.08% sodium azide and 50 mM 2-ME is supplemented with fresh 50 mM 2-ME and incubated for 1 h at RT to ensure that the Cys residue at position 4 is fully reduced. The sample is desalted using a Vivaspin 2 polyethersulfone (PES) ultrafiltration spin column (molecular weight cut-off of 5 kDa, Sartorius Stedim Biotech, part no. VS0211) and the buffer exchanged to degassed 10 mM phosphate buffer, 150 mM NaCl, 1 mM EDTA pH 7. The resulting ricin B solution is reacted overnight at 10° C. under argon with 1.1 mole equivalents of the linkage molecule containing modules (b) and (c) from Example 19(i) above. The desired delivery carrier is then purified by preparative gel filtration using a HiLoad 16/60 Superdex 75 prep grade column (GE Healthcare, part no. 17-1068-01), eluted with 50 mM sodium dihydrogen phosphate buffer, 100 mM NaCl, 2 nM EDTA pH 5.0 at a flow rate of 1 mL/min. Identification of the desired carrier peak is enabled by having pre-calibrated the SEC column with ricin B and the linker-peptide entity from Example 19(i). The product is analyzed by native gel electrophoresis and by DTT cleavage into 2 components, each of which are individually analyzed.

(iii) Preparation of the Cargo siRNA [Compound (d)]:

A Tuschl-style siRNA targeting GAPDH is synthesized, purified and analyzed exactly as described in Example 1(iii), wherein the 5′-terminus of the sense strand is modified with a 5′-(C₆-aminolinker)-phosphate-(C₆—SS—C₆)-phosphate-Cy3 entity. The primary amine is further reacted with the linker molecule SFB following the procedure in Example 1(iii) and desalted and buffer exchanged.

(iv) Coupling of a Double siRNA Cargo [2 Compounds (d)] to the Delivery Carrier [Modules (a)+(b)+(c) and 2 Linkers]:

The delivery carrier from Example 19(ii) above is reacted overnight at 10° C. with 3 mole equivalents of the linker-siRNA cargo from Example 19(iii) above in phosphate buffer pH 5. The desired module (a)+module (b)+module (c)+compounds (d) conjugate is purified by preparative SEC on a HiLoad 16/60 Superdex 75 prep grade column (GE Healthcare, part no. 17-1068-01), eluted at 1 mL/min with sterile PBS, pH 7.4. QC is performed by native gel electrophoresis and analytical SEC on a Superdex 75 10/300 GL column (GE Healthcare, part no. 17-5174-01). Further analysis is done by incubating the product with DTT or TCEP to cleave the two accessible disulfide bonds and give three molecules, each of which can be isolated by HPLC, individually characterized by ESMS and, if necessary, sequenced.

It will be apparent to one of skill in the art that the approach described within this Example may be used to attach other cargos, e.g. a nucleic acid, a protein, a peptide, a therapeutic moiety, and the like, to a delivery carrier (i.e., [module (a)+module (b)+module (c)] of the present invention.

### Example (20)

### Synthesis of DARE™ 3.02 constructs (DARE™-T-AK-SGK), Sgk1-TfR-AKDEL-siRNA (see FIG. 11), carrying fLuc and GAPDH targeted siRNAs respectively

(i) Synthesis of the Linkage Molecule Containing Modules (a), (b) and (c), viz, Sgk1-TfR-AKDEL

The [module (a) (SEQ ID NO: 111)+module (b) (SEQ ID NO: 26)+module (c) (SEQ ID NO: 66)+linker peptide (SEQ ID NO: 98)] of sequence MTVKTEAAKGTLTYSRMRGMVAILIAFMKQ-(S-G)-3-Cys-(S-G)₃-THRPPMWSPVWPA KDEL was synthesized by standard solid-phase Fmoc chemistry, deprotected in the standard fashion and purified twice by preparative reversed phase HPLC. The purity was estimated at 57-84% (due to shoulders on the back and front of the peak) by analytical reversed phase HPLC on a Vydac 218TP54 column using a gradient from 0.1% aqueous TFA to 0.1% TFA in 60% acetonitrile during 40 min, eluted at 1 mL/min. The mass measured by matrix assisted laser desorption ionization mass spectroscopy (MALDI-MS) in positive ion mode was 6346.81 Da for M+H⁺; the calculated mass of C₂₇₅H₄₄₂N₇₈O₈₂S₆ is 6345.41 Da. The cysteine thiol was then activated by reaction of the purified peptide (50 mg, ca. 7 μmol) with 5-nitro-2-[(5-nitropyridin-2-yl)disulfanyl]pyridine (6.2 mg, 20 μmol; from Sigma-Aldrich, catalog #43765) in pyridine (5 mL) for 2 h at room temperature with stirring, to give 11 mg of the desired MTVKTEAAKGTLTYSRMRGMVAILIAFMKQ-(S-G)₃-Cys(pNPys)-(S-G)₃-THRPPMWSPV WPAKDEL after two preparative RP-HPLC purifications. The purity of the activated peptide was 78.8% by reversed phase HPLC. MALDI-TOF MS showed the correct M+H⁺ ion at m/z 6500.64; the calculated mass for C₂₈₀H₄₄₄N₈₀O₈₄S₇ is 6499.56 Da.

(ii) Preparation of the siRNA Cargo Compounds (d)

A double stranded RNA molecule comprised of two 21 mer strands, with a double stranded region of 19 nucleotides in length and 2 nucleotides overhanging at the 3′-end of each strand, and targeting glyceraldehyde 3-phosphate dehydrogenase (GAPDH), wherein the sense strand comprises 5′-CCAuCUUCCAGGAGCgAGAuu (SEQ ID NO: 194), wherein lowercase u or g represents a 2′-O-methylribonucleotide; and the antisense strand comprises 5′-UCUCGCUCCUGgAAGAuGGdTdT (SEQ ID NO: 195), wherein lowercase u or g represents a 2′-O-methylribonucleotide and wherein the antisense strand has a 5′-phosphate and deoxynucleotides at its 3′-end (dNdN), was synthesized such that the 5′-terminus of the sense strand was modified with a 5-(C6-SS—C6 spacer)-phosphate-Cy3 moiety. In addition, a double stranded RNA molecule comprised of two 21 mer strands, with a double stranded region of 19 nucleotides in length and 2 nucleotides overhanging at the 3′-end of each strand, and targeting firefly luciferase (fLuc), wherein the sense strand comprises 5′-CUUACgCUGAGuACUUCGAuu (SEQ ID NO: 197), wherein lowercase u or g represents a 2′-O-methylribonucleotide; and the antisense strand comprises 5′-UCGAAGUACUC AgCGUAAgdTdG (SEQ ID NO: 198), wherein lowercase g represents a 2′-O-methylribonucleotide and wherein the antisense strand has a 5′-phosphate and deoxynucleotides at its 3′-end (dNdN), was synthesized such that the 5′-terminus of the sense strand was modified with a 5-(C6-SS—C6 spacer)-phosphate-Cy3 moiety. The four HPLC-purified individual single strands were all analyzed by HPLC and MALDI-TOF MS. In order to prepare the two duplexes for the disulfide exchange reaction with the activated linkage molecule containing modules (a), (b) and (c), 50 A₂₆₀ units of each duplex was dissolved in 0.5 mL of sterile 0.2 M aqueous sodium acetate, pH 6 containing 100 mM dithiothreitol (DTT) and kept at 37° C. for 2 h to cleave the disulfide bond. The solutions were then desalted using degassed water as eluent and lyophilized.

(iii) Coupling of the siRNA Cargo [Compound (d)] to the Delivery Carrier [Modules (a)+(b)+(c) and Linker]

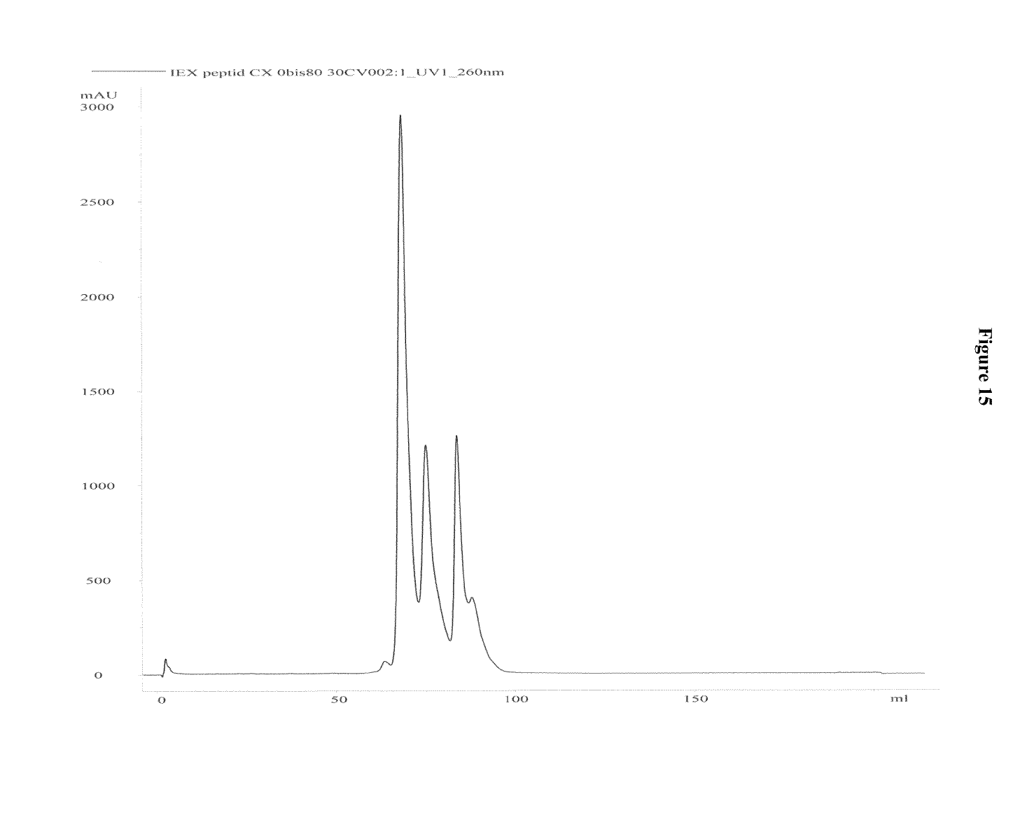

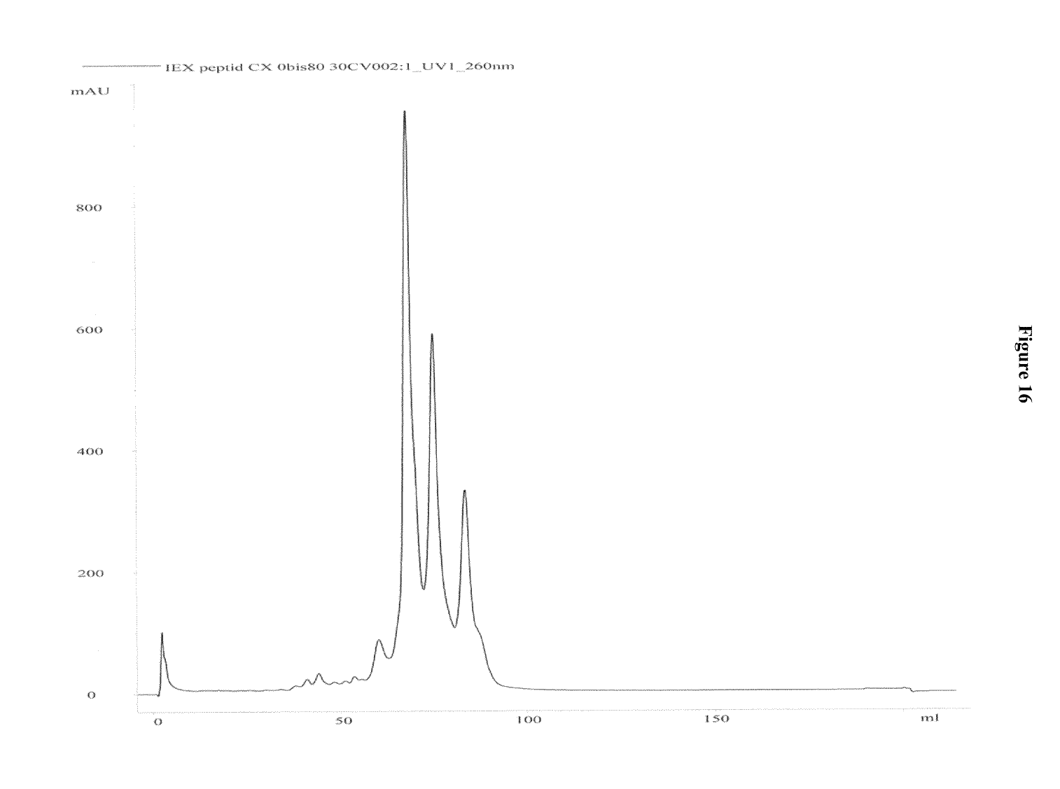

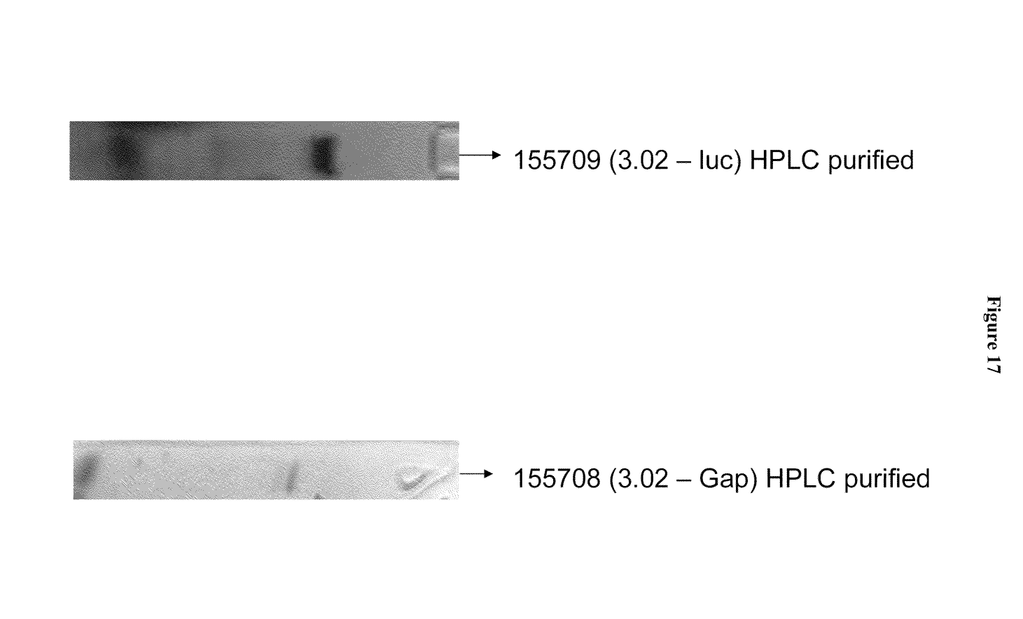

fLuc-siRNA (10 A₂₆₀ units, ˜25 nmol) from Example 20(ii) above was dissolved in 100 μL of 8 M guanidinium chloride in sterile phosphate buffered saline (PBS), pH 7.4 under argon. MTVKTEAAKGTLTYSRMRGMVAILIAFMKQ-(S-G)-3-Cys(pNPys)-(S-G)-3-THRPPMWSP VWPAKDEL (0.5 mg, ˜72 nmol) from Example 20(i) above was dissolved in 100 μL of 8 M guanidinium chloride in degassed sterile water. The peptide solution was added to the fLuc-siRNA solution and the reaction was allowed to proceed for 17 h at 22° C. The solution was then diluted to 1 mL with sterile 50 mM ammonium acetate and loaded into a spin column (0.5 mL, Amicon Ultra with an Ultracel 10 kDa membrane). The column was washed once with 50 mM ammonium acetate followed by water. The desalted sample was removed, lyophilized and then dissolved in 0.5 mL of sterile 25 mM Tris-HCl buffer, pH 7.4 containing 6 M urea (buffer A) and loaded onto a 1 mL Resource Q anion-exchange HPLC column (GE Healthcare, part no. 17-1177-01). The column was eluted with a linear gradient from 0-80% B in 180 column volumes (CV) using a flow rate of 3 mL/min. Buffer B was 25 mM Tris-HCl, 1 M sodium bromide and 6 M urea, pH 7.4 using an Akta purifier HPLC (GE Healthcare). The column effluent was monitored at 260 nm and 550 nm (Cy3 absorbance) and three peaks were observed, the first (major) peak was identified as the desired conjugate by mass spectroscopy. The preparative anion-exchange HPLC trace is shown in FIG. 15. An identical experiment was performed for the GAPDH-siRNA, and the preparative anion-exchange HPLC trace is shown in FIG. 16. The product containing peaks were exhaustively desalted using a spin column and then lyophilized. The yield of the two purified DARE™ 3.02 constructs was in the range of 3-7 nmol. FIG. 17 shows 15% PAGE gels of the fLuc-siRNA and GAPDH-siRNA containing DARE™ 3.02 constructs, performed at 220 V and 25 mA with a running time of 1-1.5 h, using a precast 8×6.5 cm gel (Biostep, part no. 95-70-181) and standard Tris-borate running buffer containing 6 M urea. Confirmation of construct identity was performed by MALDI-TOF mass spectroscopy on a Voyager instrument, see FIGS. 18 (3.03-fLuc) and 19 (3.02-GAPDH).

### Example (21)

### Synthesis of DARE™ Delivery System Delivery Modules and Preparation of the Modules-siRNA Conjugate CTB-COX2-AKDEL-siRNA (DARE™ Delivery Vehicle Design 2.23)

(i) Synthesis of the Linkage Molecule Containing Delivery Modules (b) and (c):

A [“module (b)+module (c)”+linker] molecule: H₂N—C(S-G)₃(DprAoa)(S-G)₃NASSSRSGLDDINPTVLLKAKDEL-OH [“module (b)+module (c)” comprise SEQ ID NO: 3; COX2-AKDEL] is synthesized commercially by standard solid-phase Fmoc peptide chemistry, deprotected in the standard fashion and purified by reversed phase High Performance Liquid Chromatography (HPLC) to a purity of >95%. Fmoc-Dpr(Boc-Aoa)-OH (Novabiochem product no. 04-12-1185) is used to introduce the N-β-aminooxyacetyl L-diaminopropionyl residue. Quality control (QC) of the purified peptide is done by amino acid analysis, electrospray mass spectroscopy (ESMS) and analytical reversed phase HPLC.

(ii) Functionalization of Module (a) (Method B):

To functionalize module (a), 5.8 mg (100 nmol of pentamer) of recombinant cholera toxin subunit B [(CTB; SEQ ID NO: 117), obtained from SBL Vaccin AB, Matfors, Sweden, see www.rctb.net/theproduct.htm] in 0.5 mL of sterile PBS is mixed with a fresh solution of 0.52 mg (1 mmol) of sulfosuccinimidyl 6-[3′-(2-pyridyldithio)-propionamido] hexanoate (sulfo-LC-SPDP) in 75 μL of sterile PBS pH 7.4 in a sterile 1 mL Eppendorf tube and kept 1.5 h at room temperature with occasional mixing. The solution is then desalted and buffer exchanged against degassed 10 mM sodium phosphate buffer, 150 mM NaCl, pH 7.4 (PBS) containing 250 μM EDTA using a Vivaspin 6 centrifugal concentrator (10 kDa MWCO, Sartorius Stedim Biotech product no. VS0601). Initially the functionalized CTB solution is diluted with 5 mL of PBS plus 250 μM EDTA concentrated to a volume of 0.5 mL and then washed with 6×5 mL of PBS plus 250 μM EDTA at room temperature, each time reducing the volume to 0.5 mL. In order to determine the 2-pyridyl-disulfide loading of the functionalized CTB a small aliquot (10 μL) of the solution is diluted to 1 mL with PBS and the absorbance at 343 nm measured using a quartz cuvette with a 1 cm pathlength. A 10 μL aliquot of a 15 mg/mL solution of dithiothreitol (DTT) in PBS is added to the cuvette, the contents are mixed carefully and the absorbance at 343 nm is measured after 15 min at room temperature, enabling the amount of pyridine-2-thione released to be quantitated (ε₃₄₃ₙₘ is 8080 M⁻¹ cm⁻¹, i.e. 1 mmol is equivalent to 8.08 A₃₄₃ units). The 2-pyridyl-disulfide loading of the CTB is generally around 5 μmol per μmol of pentamer. It is clear to one skilled in the art that the loading can be reduced by reducing the excess of sulfo-LC-SPDP used in the functionalization.

(iii) Synthesis of the Delivery Carrier Comprising Functionalized Module (a), Modules (b) and (c) and the Linker (Method B):

250 μL of solution containing functionalized CTB (50 nmol) from Example 21(ii) above is reacted for 18 h at RT under nitrogen with 1.0 mg (250 nmol) of the linkage molecule containing modules (b) and (c) from Example 21(i) above dissolved in 750 μL of 100 mM phosphate buffer pH 7.4. Following a brief centrifugation the desired carrier [modules (a)+(b)+(c)] is then purified by preparative gel filtration [Size Exclusion Chromatography (SEC)] using a HiLoad 16/60 Superdex 75 prep grade column (GE Healthcare, part no. 17-1068-01) eluted with PBS at a flow rate of 1 mL/min. Identification of the desired carrier peak is enabled by having calibrated the SEC column with CTB and with the linker-peptide entity from Example 21(i). Product containing fractions are pooled and concentrated to a volume of 0.5 mL using a Vivaspin 20 protein concentrator (10 kDa MWCO, Sartorius Stedim Biotech product no. VS2001).

(iv) Preparation of the Cargo siRNA [Compound (d)]:

The cargo siRNA [compound (d)] is prepared as described in Example 1(iii) above.

(v) Coupling of the Cargo [Compound (d)] to the Delivery Carrier [Modules (a)+(b)+(c) and a Linker]:

The carrier (40 nmol, based on CTB pentamer) from Example 21(iii) above in 500 μL of 100 mM phosphate buffer containing 100 mM aniline pH 7 is mixed with 200 nmol of the linker-siRNA component (cargo) from Example 21(iv) above in 500 μL of 100 mM phosphate buffer containing 100 mM aniline and kept for 24 h at RT. The desired conjugate is purified by preparative SEC on a HiLoad 16/60 Superdex 200 prep grade column (GE Healthcare, part no. 17-1069-01) eluted at 1 mL/min with sterile PBS, pH 7.4. The column effluent is monitored at 260 nm and 550 nm. Calibration of the column is carried out prior to the preparative purification using the individual reaction components. Those fractions containing the desired conjugate are combined and concentrated by ultrafiltration using a Vivaspin 20 concentrator (30 kDa MWCO, Sartorius Stedim Biotech product no. VS2021) and the final concentrate is stored at 4° C. QC is performed by native gel electrophoresis and analytical SEC on a Superdex 200 10/300 GL column (GE Healthcare, part no. 17-5175-01).

### Example (22)

### Synthesis of DARE™ Delivery System Delivery Modules and Preparation of the Modules-siRNA Conjugate DARE™-mPT-KDEL with a Disulfide Bond in the Linkage Between Module (a)+(c) and Module (b)

(i) Synthesis of the Linkage Molecule Containing Delivery Module (b):

A [“module (b)”+linker] molecule: H₂N—C(S-G)₃(DprAoa)(S-G)₃AKDEL-OH [“module (b)” comprises SEQ ID NO: 25; KDEL and linker comprises SEQ ID NO: 98] is synthesized commercially by standard solid-phase Fmoc peptide chemistry, deprotected in the standard fashion and purified by reversed phase High Performance Liquid Chromatography (HPLC) to a purity of >92%. Fmoc-Dpr(Boc-Aoa)-OH (Novabiochem product no. 04-12-1185) is used to introduce the N-β-aminooxyacetyl L-diaminopropionyl residue. Quality control (QC) of the purified peptide is done by amino acid analysis, electrospray mass spectroscopy (ESMS) and analytical reversed phase HPLC.

(ii) Functionalization of Module (a)+(c):

To functionalize module (a)+(c), 10.6 mg (100 nmol) of recombinant non-toxic mutant pertussis toxin (mPT carrying a mutation in the active site of the A subunit to render it non-toxic) in 1 mL of sterile PBS is mixed with a fresh solution of 0.26 mg (0.5 mmol) of sulfosuccinimidyl 6-[3′-(2-pyridyldithio)-propionamido] hexanoate (sulfo-LC-SPDP) in 100 μL of sterile PBS pH 7.4 in a sterile 2 mL Eppendorf tube and kept 1.5 h at room temperature with occasional mixing. The solution is then desalted and buffer exchanged against degassed 10 mM sodium phosphate buffer, 150 mM NaCl, pH 7.4 (PBS) containing 250 μM EDTA using a Vivaspin 6 centrifugal concentrator (30 kDa MWCO, Sartorius Stedim Biotech product no. VS0621). Initially the functionalized mPT solution is diluted with 5 mL of PBS plus 250 μM EDTA concentrated to a volume of 1 mL and then washed with 6×5 mL of PBS plus 250 μM EDTA at room temperature, each time reducing the volume to 1 mL. In order to determine the 2-pyridyl-disulfide loading of the functionalized mPT a small aliquot (20 μL) of the solution is diluted to 1 mL with PBS and the absorbance at 343 nm measured using a quartz cuvette with a 1 cm pathlength. A 10 μL aliquot of a 15 mg/mL solution of dithiothreitol (DTT) in PBS is added to the cuvette, the contents are mixed carefully and the absorbance at 343 nm is measured after 15 min at room temperature, enabling the amount of pyridine-2-thione released to be quantitated (E343nm is 8080 M⁻¹ cm⁻¹, i.e. 1 mmol is equivalent to 8.08 A₃₄₃ units). The 2-pyridyl-disulfide loading of the mPT is generally around 2.5 μmol per μmol of mPT. It is clear to one skilled in the art that the loading can be altered by increasing or reducing the excess of sulfo-LC-SPDP used in the functionalization.

(iii) Synthesis of the Delivery Carrier Comprising Functionalized Module (a)+(c), Module (b) and the Linker:

1 mL of solution containing functionalized mPT (100 nmol) from Example 22(ii) above is reacted for 18 h at RT under nitrogen with 0.46 mg (250 nmol) of the linkage molecule containing module (b) from Example 22(i) above dissolved in 250 μL of 100 mM phosphate buffer pH 7.4. Following a brief centrifugation the desired carrier [modules (a)+(c)+(b)] is then purified by preparative gel filtration [Size Exclusion Chromatography (SEC)] using a HiLoad 16/60 Superdex 200 prep grade column (GE Healthcare, part no. 17-1069-01) eluted with PBS at a flow rate of 1 mL/min. Identification of the desired carrier peak is enabled by having calibrated the SEC column with mPT and with the linker-peptide entity from Example 22(i). Product containing fractions are pooled and concentrated to a volume of 0.5 mL using a Vivaspin 20 protein concentrator (30 kDa MWCO, Sartorius Stedim Biotech product no. VS0621).

(iv)Preparation of the Cargo siRNA [Compound (d)]:

The cargo siRNA [compound (d)] is prepared as described in Example 1(iii) above.

(v) Coupling of the Cargo [Compound (d)] to the Delivery Carrier [Modules (a)+(c)+(b) and a Linker]:

The carrier (40 nmol) from Example 22(iii) above in 500 μL of 100 mM phosphate buffer containing 100 mM aniline pH 7 is mixed with 120 nmol of the linker-siRNA component (cargo) from Example 22(iv) above in 500 μL of 100 mM phosphate buffer containing 100 mM aniline and kept for 24 h at RT. The desired conjugate is purified by preparative SEC on a HiLoad 16/60 Superdex 200 prep grade column (GE Healthcare, part no. 17-1069-01) eluted at 1 mL/min with sterile PBS, pH 7.4. The column effluent is monitored at 260 nm and 550 nm. Calibration of the column is carried out prior to the preparative purification using the individual reaction components. Those fractions containing the desired conjugate are combined and concentrated by ultrafiltration using a Vivaspin 20 concentrator (30 kDa MWCO) and the final concentrate is stored at 4° C. QC is performed by native gel electrophoresis and analytical SEC on a Superdex 200 10/300 GL column (GE Healthcare, part no. 17-5175-01).

### Example (23)

### Synthesis of DARE™ Delivery System Delivery Modules and Preparation of the Modules-siRNA Conjugate DARE™-mPT-KDEL with a Non-Cleavable Thioether Bond in the Linkage Between Module (a)+(c) and Module (b)

(i) Synthesis of the Linkage Molecule Containing Delivery Module (b):

A [“module (b)”+linker] molecule: H₂N—C(S-G)₃(DprAoa)(S-G)₃AKDEL-OH [“module (b)” comprise SEQ ID NO: 25; KDEL and linker comprises SEQ ID NO: 98] is synthesized commercially by standard solid-phase Fmoc peptide chemistry, deprotected in the standard fashion and purified by reversed phase High Performance Liquid Chromatography (HPLC) to a purity of >92%. Fmoc-Dpr(Boc-Aoa)-OH (Novabiochem product no. 04-12-1185) is used to introduce the N-β-aminooxyacetyl L-diaminopropionyl residue. Quality control (QC) of the purified peptide is done by amino acid analysis, electrospray mass spectroscopy (ESMS) and analytical reversed phase HPLC.

(ii) Functionalization of Module (a)+(c):

To functionalize module (a)+(c), 10.6 mg (100 nmol) of recombinant non-toxic mutant pertussis toxin (mPT carrying a mutation in the active site of the A subunit to render it non-toxic) in 1 mL of sterile 100 mM sodium phosphate, 150 mM sodium chloride, pH 7.2 is mixed with 100 μL of a fresh solution of 2.18 mg (5 mmol) of sulfosuccinimidyl 4-(N-maleimidomethyl)cyclohexane-1-carboxylate (sulfo-SMCC) in 1 mL of sterile 100 mM sodium phosphate buffer pH 7.2 in a sterile 2 mL Eppendorf tube and kept 1 h at room temperature with occasional mixing. The solution is then desalted and buffer exchanged against degassed 100 mM sodium phosphate buffer, 150 mM NaCl, pH 7.2 containing 250 μM EDTA using a Vivaspin 6 centrifugal concentrator (30 kDa MWCO, Sartorius Stedim Biotech product no. VS0621). Initially the functionalized mPT solution is diluted with 5 mL of sterile 100 mM sodium phosphate, 150 mM sodium chloride plus 250 μM EDTA concentrated to a volume of 1 mL and then washed with 6×5 mL of sterile 100 mM sodium phosphate, 150 mM sodium chloride pH 7.2 containing 250 μM EDTA at room temperature, each time reducing the volume to 1 mL. The maleimido loading of the mPT is generally around 2 μmol per μmol of mPT. It is clear to one skilled in the art that the loading can be altered by increasing or reducing the excess of sulfo-SMCC used in the functionalization.

(iii) Synthesis of the Delivery Carrier Comprising Functionalized Module (a)+(c), Module (b) and the Linker:

1 mL of solution containing functionalized mPT (100 nmol) from Example 23(ii) above is reacted overnight at RT under nitrogen with 0.37 mg (200 nmol) of the linkage molecule containing module (b) from Example 23(i) above dissolved in 250 μL of 100 mM phosphate buffer pH 7.2. Following a brief centrifugation the desired carrier [modules (a)+(c)+(b)] is then purified by preparative gel filtration [Size Exclusion Chromatography (SEC)] using a HiLoad 16/60 Superdex 200 prep grade column (GE Healthcare, part no. 17-1069-01) eluted with PBS pH 7 at a flow rate of 1 mL/min. Identification of the desired carrier peak is enabled by having calibrated the SEC column with mPT and with the linker-peptide entity from Example 23(i). Product containing fractions are pooled and concentrated to a volume of 0.5 mL using a Vivaspin 20 protein concentrator (30 kDa MWCO, Sartorius Stedim Biotech product no. 2021).

(iv) Preparation of the Cargo siRNA [Compound (d)]:

The cargo siRNA [compound (d)] is prepared as described in Example 1(iii) above.

(v) Coupling of the Cargo [Compound (d)] to the Delivery Carrier [Modules (a)+(c)+(b) and a Linker]:

The carrier (40 nmol) from Example 23(iii) above in 500 μL of 100 mM phosphate buffer containing 100 mM aniline pH 7 is mixed with 120 nmol of the linker-siRNA component (cargo) from Example 23(iv) above in 500 μL of 100 mM phosphate buffer pH 7 containing 100 mM aniline and kept for 24 h at RT. The desired conjugate is purified by preparative SEC on a HiLoad 16/60 Superdex 200 prep grade column (GE Healthcare, part no. 17-1069-01) eluted at 1 mL/min with sterile PBS, pH 7.4. The column effluent is monitored at 260 nm and 550 nm. Calibration of the column is carried out prior to the preparative purification using the individual reaction components. Those fractions containing the desired conjugate are combined and concentrated by ultrafiltration using a Vivaspin 20 concentrator (30 kDa MWCO, Sartorius Stedim Biotech product no. VS2021) and the final concentrate is stored at 4° C. QC is performed by native gel electrophoresis and analytical SEC on a Superdex 200 10/300 GL column (GE Healthcare, part no. 17-5175-01).

### Example (24)

### Synthesis of DARE™ Delivery System Delivery Modules and Preparation of the Modules-siRNA Conjugate DARE™-mCT

(i) Synthesis of the Linkage Molecule Containing Targeting Module (a):

A [“module (a)”+linker] molecule: H₂N-TPQNITDLCAEYHNTQIYTLNDKIFSYTES-LAGKREMAIITFKNGAIFQVEVPGSQHIDSQKKAIERMKDTLRIAYLTEAKVEKLCV-WNNKTPHAIAAISMANSGSGSG(DprAoa)-OH [“module (a)”+linker comprise SEQ ID NO: 224] is synthesized commercially by solid-phase Fmoc peptide chemistry, deprotected in the standard fashion, and the crude product is purified by reversed phase High Performance Liquid Chromatography (HPLC) to a purity of >80%. Following purification the two cysteines are oxidized to form an intramolecular disulfide bond and the folded peptide is purified once more. Fmoc-Dpr(Boc-Aoa)-OH (Novabiochem product no. 04-12-1185) is used to introduce the N-β-aminooxyacetyl L-diaminopropionyl residue. In solution at pH<3.2 in the presence of 6.5 M the peptide stays as a monomer (see Finkelstein, R. A et al. In J. Immunol., 1974, 113, 145-150). Quality control (QC) of the purified peptide is done by amino acid analysis, electrospray mass spectroscopy (ESMS) and analytical reversed phase HPLC.

(ii) Preparation of Mutant Cholera Toxin Subunit A, Module “(c)+(b)”:

A recombinant non-toxic mutant cholera toxin subunit A (mCTA carrying a mutation in the active site of the A subunit to render it non-toxic) is obtained by fermentation.

(iii) Synthesis of the Delivery Carrier Comprising Functionalized Module “(a)+Linker” and Module “(c)+(b)”:

30 nmol of non-toxic mutant cholera toxin subunit A from Example 24(ii) above and 125 nmol of the linkage molecule containing targeting module (a) are mixed together in 2 mL of sterile 100 mM glycine-HCl buffer, 6.5 M urea, pH 3.2. This solution is then dialyzed against sterile PBS, pH 7 so as to assemble the mutant CT holotoxin molecule containing one mutant A subunit and five B subunits, each of which carries a C-terminal β-aminooxyacetyl L-diaminopropionyl residue. The delivery carrier is purified by preparative SEC on a HiLoad 16/60 Superdex 200 prep grade column (GE Healthcare, part no. 17-1069-01) eluted at 1 mL/min with sterile PBS, pH 7.4. The column effluent is monitored at 220 and 280 nm. Those fractions containing the desired delivery carrier are combined and concentrated to 0.5 mL by ultrafiltration using a Vivaspin 15R concentrator (30 kDa MWCO, Sartorius Stedim Biotech product no. VS15RH21) and the final concentrate is stored at 4° C. QC is performed by native gel electrophoresis and analytical SEC on a Superdex 200 10/300 GL column (GE Healthcare, part no. 17-5175-01).

(iv) Preparation of the Cargo siRNA [Compound (d)]:

The cargo siRNA [compound (d)] is prepared as described in Example 1(iii) above.

(v) Coupling of the Cargo [Compound (d)] to the Delivery Carrier Comprising 5× “Module (a)+Linker” and Module “(c)+(b)”:

The delivery carrier (20 nmol) from Example 24(iii) above in 500 μL of sterile 100 mM phosphate buffer containing 100 mM aniline pH 7 is mixed with 120 nmol of the linker-siRNA component (cargo) from Example 24(iv) above in 500 μL of sterile 100 mM phosphate buffer pH 7 containing 100 mM aniline and kept for 24 h at RT. The desired conjugate is purified by preparative SEC on a HiLoad 16/60 Superdex 200 prep grade column (GE Healthcare, part no. 17-1069-01) eluted at 1 mL/min with sterile PBS, pH 7.4. The column effluent is monitored at 260 nm and 550 nm. Calibration of the column is carried out prior to the preparative purification using the individual reaction components. Those fractions containing the desired conjugate are combined and concentrated by ultrafiltration using a Vivaspin 20 concentrator (30 kDa MWCO, Sartorius Stedim Biotech product no. VS2021) and the final concentrate is stored at 4° C. QC is performed by native gel electrophoresis and analytical SEC on a Superdex 200 10/300 GL column (GE Healthcare, part no. 17-5175-01).

### Example (25)

### Synthesis of DARE™ Delivery System Delivery Modules and Preparation of the Modules-siRNA Conjugate DARE™-CTB-COX2-KDEL with a PEST Motif in the Linkage Between the Delivery Carrier and the siRNA Cargo [Compound (d)]

(i) Synthesis of the Linkage Molecule Containing Delivery Modules (b) and (c):

A “module (b)+module (c)+linker” molecule: N-acetyl-CSGSGSG-bLys-SGSGSG-NASSSRSGLDDINPTVLLKAKDEL-OH, whereby the c-amino group of the branching Lys residue carries in addition the sequence 12-(aminooxy)dodecanoyl-SGKDSSPSSSPSPK-SGSGSG [“module (b)+module (c)+linker” comprise SEQ ID NO: 225; COX2-KDEL-PEST] is synthesized commercially by solid-phase Fmoc peptide chemistry. The N-terminal 12-(aminooxy)dodecanoyl moiety is introduced using 12-(Boc-aminooxy)-dodecanoic acid (Bachem, product no. A-4720). The branch point lysine residue is introduced using Fmoc-Lys(ivDde)-OH (Merck Novabiochem, product no. 8520820001). Peptide synthesis is done up to and including the branch point orthogonally protected lysine residue which is then selectively deprotected with 20% piperidine and the first branch is synthesized with an N-terminal acetyl cap on the Cys residue. The ivDde protecting group on the Lys c-amino group is then removed with 2% hydrazine in DMF enabling synthesis of the second branch terminating in a 12-(Boc-aminooxy)dodecanoyl moiety. Deprotection is performed in the standard fashion and the crude product is purified by reversed phase HPLC to give a purity >95%. Quality control (QC) of the purified peptide is done by amino acid analysis, electrospray mass spectroscopy (ESMS) and analytical reversed phase HPLC.

(ii) Functionalization of Module (a):

To functionalize module (a), 5.8 mg (100 nmol of pentamer) of recombinant cholera toxin subunit B [(CTB; SEQ ID NO: 117), obtained from SBL Vaccin AB, Matfors, Sweden, see www.rctb.net/theproduct.htm] in 0.5 mL of sterile PBS is mixed with a fresh solution of 0.52 mg (1 μmol) of sulfosuccinimidyl 6-[3′-(2-pyridyldithio)-propionamido] hexanoate (sulfo-LC-SPDP) in 75 μL of sterile PBS pH 7.4 in a sterile 1 mL Eppendorf tube and kept 1.5 h at room temperature with occasional mixing. The solution is then desalted and buffer exchanged against degassed 10 mM sodium phosphate buffer, 150 mM NaCl, pH 7.4 (PBS) containing 250 μM EDTA using a Vivaspin 6 centrifugal concentrator (10 kDa MWCO, Sartorius Stedim Biotech product no. VS0601). Initially the functionalized CTB solution is diluted with 5 mL of PBS plus 250 μM EDTA concentrated to a volume of 0.5 mL and then washed with 6×5 mL of PBS plus 250 μM EDTA at room temperature, each time reducing the volume to 0.5 mL. In order to determine the 2-pyridyl-disulfide loading of the functionalized CTB a small aliquot (10 μL) of the solution is diluted to 1 mL with PBS and the absorbance at 343 nm measured using a quartz cuvette with a 1 cm pathlength. A 10 μL aliquot of a 15 mg/mL solution of dithiothreitol (DTT) in PBS is added to the cuvette, the contents are mixed carefully and the absorbance at 343 nm is measured after 15 min at room temperature, enabling the amount of pyridine-2-thione released to be quantitated (ε₃₄₃ₙₘ is 8080 M⁻¹ cm⁻¹, i.e. 1 μmol is equivalent to 8.08 A₃₄₃ units). The 2-pyridyl-disulfide loading of the CTB is generally around 5 μmol per μmol of pentamer. It is clear to one skilled in the art that the loading can be reduced by reducing the excess of sulfo-LC-SPDP used in the functionalization.

(iii) Synthesis of the Delivery Carrier Comprising Functionalized Module (a), Modules (b) and (c) and the Linker:

250 μL of solution containing functionalized CTB (50 nmol) from Example 25(ii) above is reacted for 18 h at RT under nitrogen with 1.44 mg (250 nmol) of the linkage molecule containing modules (b) and (c) from Example 25(i) above dissolved in 750 μL of 100 mM phosphate buffer pH 7.4. Following a brief centrifugation the desired carrier [modules (a)+(b)+(c)] is then purified by preparative gel filtration [Size Exclusion Chromatography (SEC)] using a HiLoad 16/60 Superdex 75 prep grade column (GE Healthcare, part no. 17-1068-01) eluted with PBS at a flow rate of 1 mL/min. Identification of the desired carrier peak is enabled by having calibrated the SEC column with CTB and with the linker-peptide entity from Example 25(i). Product containing fractions are pooled and concentrated to a volume of 0.5 mL using a Vivaspin 20 protein concentrator (10 kDa MWCO, Sartorius Stedim Biotech product no. VS2001).

(iv) Preparation of the Cargo siRNA [Compound (d)]:

A double stranded RNA molecule comprised of two 21 mer strands, with a double stranded region of 19 nucleotides in length and 2 nucleotides overhanging at the 3′ end of each strand, and targeting glyceraldehyde 3-phosphate dehydrogenase (GAPDH), wherein the sense strand comprises CCAuCUUCCAGGAGCgAGAuu (SEQ ID NO: 194), wherein lowercase u or g represents a 2′-O-Me-modified nucleotide; and the antisense strand comprises UCUCGCUCCUGgAAGAuGGdTdG (SEQ ID NO: 195), wherein lowercase u or g represents a 2′-O-Me-modified nucleotide and wherein the antisense strand has a 5′-phosphate and deoxynucleotides at its 3′ end (dNdN), is synthesized such that the 5P-terminus of the sense strand is modified with 5′-(C6 aminolinker)-phosphate-Cy3. The Cy3 dye is for tracking purposes. The single strands are analyzed by ESMS and analytical HPLC for QC prior to annealing. The desalted lyophilized siRNA (200 nmol) is dissolved in 1 mL of sterile 100 mM sodium tetraborate buffer pH 8.5 and reacted with 200 μL of a fresh solution of 3.49 mg (10 μmol) of sulfosuccinimidyl 4-formylbenzoate (sulfo-S-4FB, SoluLink, product no. S-1008-105) dissolved in 500 μL of sterile 100 mM sodium tetraborate buffer, pH 8.5 for 3 h at RT. The siRNA bearing a benzaldehyde function is then desalted and buffer exchanged against degassed 100 mM sodium phosphate buffer, 150 mM NaCl, 500 μM EDTA, pH 7 using a Vivaspin 15 centrifugal concentrator (5 kDa MWCO, Sartorius Stedim Biotech product no. VS1511). Initially the benzaldehyde modified siRNA solution is diluted with 8.5 mL of sterile degassed 100 mM sodium phosphate, 150 mM sodium chloride, 500 μM EDTA, pH 7 concentrated to a volume of 1.5 mL and then washed with 5×8.5 mL of sterile degassed 100 mM sodium phosphate, 150 mM sodium chloride, 500 μM EDTA, pH 7 at room temperature, each time reducing the volume to 1.5 mL. QC of the benzaldehyde modified siRNA is done by ESMS and analytical HPLC. A small aliquot of the sample is analyzed for the presence of the aldehyde moiety by reaction with an excess of Cascade Blue hydrazide (Molecular Probes, catalog no. C-687) in buffer at pH 5, desalted by ethanol precipitation and analyzed by native anion-exchange HPLC on a MonoQ column (GE Healthcare) using multiwavelength detection (260 nm for the RNA, 399 nm for the Cascade Blue and 550 nm for the Cy3).

(v) Coupling of the Cargo [Compound (d)] to the Delivery Carrier [Modules (a)+(b)+(c) and a Linker]:

The carrier (40 nmol, based on CTB pentamer) from Example 25(iii) above in 500 μl of 100 mM phosphate buffer containing 10 mM aniline pH 7 is mixed with 200 nmol of the linker-siRNA component (cargo) from Example 25(iv) above in 500 μl of 100 mM phosphate buffer containing 10 mM aniline and kept for 24 h at RT. The desired conjugate is purified by preparative SEC on a HiLoad 16/60 Superdex 200 prep grade column (GE Healthcare, part no. 17-1069-01) eluted at 1 mL/min with sterile PBS, pH 7.4. The column effluent is monitored at 260 nm and 550 nm. Calibration of the column is carried out prior to the preparative purification using the individual reaction components. Those fractions containing the desired conjugate are combined and concentrated by ultrafiltration using a Vivaspin 20 concentrator (30 kDa MWCO, Sartorius Stedim Biotech product no. VS2021) and the final concentrate is stored at 4° C. QC is performed by native gel electrophoresis and analytical SEC on a Superdex 200 10/300 GL column (GE Healthcare, part no. 17-5175-01).

Synthesising DARE Constructs with Toxins

The following Table 2 indicates how preferred DARE-Constructs of the invention, wherein toxins of the AB₅-type or the AB-type are used, are generated. The reaction types used are to be found in FIGS. 22A and 22B. If both the A and B subunits of a given toxin are used in a DARE construct, it is possible to couple the other components of the DARE construct either to the A subunit or to one B subunit or to two or more B subunits. In these cases, the respective A and B subunits are themselves connected via covalent bonds, usually via Cys-residues in both protein subunit chains. For these preferred DARE constructs the preferred compound (d) is a nucleic acid, preferably a siRNA.

TABLE 2

| Toxin class | Example procedure | Reaction types used |
|---|---|---|
| AB₅ type: | | |
| Cholera toxin B subunit | 21 or 23 | I, II & V or I, III & V |
| Shiga toxins: | | |
| Shigella species Stx1a B subunit | 21 or 23 | I, II & V or I, III & V |
| E. coli Stx1b (VT1b) B subunit | 21 or 23 | I, II & V or I, III & V |
| E. coli Stx1c (VT1c) B subunit | 21 or 23 | I, II & V or I, III & V |
| E. coli Stx1d (VT1d) B subunit | 21 or 23 | I, II & V or I, III & V |
| E. coli Stx2a (VT2a) B subunit | 21 or 23 | I, II & V or I, III & V |
| E. coli Stx2b (VT2b) B subunit | 21 or 23 | I, II & V or I, III & V |
| E. coli Stx2c (VT2c) B subunit | 21 or 23 | I, II & V or I, III & V |
| E. coli Stx2d (VT2d) B subunit | 21 or 23 | I, II & V or I, III & V |
| E. coli Stx2e (VT2e) B subunit | 21 or 23 | I, II & V or I, III & V |
| E. coli Stx2f (VT2f) B subunit | 21 or 23 | I, II & V or I, III & V |
| E. coli Stx2g (VT2g) B subunit | 21 or 23 | I, II & V or I, III & V |
| Heat-labile enterotoxin B subunit | 21 or 23 | I, II & V or I, III & V |
| Heat-labile enterotoxin B subunit | 21 or 23 | I, II & V or I, III & V |
| (LT-B, porcine), B subunit | | |
| Heat-labile enterotoxin IIA, | 21 or 23 | I, II & V or I, III & V |
| B subunit (LT-IIA) | | |
| Heat-labile enterotoxin IIB, | 21 or 23 | I, II & V or I, III & V |
| B subunit (LT-IIB) | | |
| Pertussis toxin, | 21 or 23 | I, II & V or I, III & V |
| heteropentameric B subunit | | |
| E. coli subtilase cytotoxin B subunit | 21 or 23 | I, II & V or I, III & V |
| Cholera toxin, | 21 or 23 | I, II & V or I, III & V |
| mutant A subunit | | |
| Shiga toxins: | | |
| Stx1a mutant A subunit | 21 or 23 | I, II & V or I, III & V |
| Stx1b (VT1b) mutant A subunit | 21 or 23 | I, II & V or I, III & V |
| Stx1c (VT1c) mutant A subunit | 21 or 23 | I, II & V or I, III & V |
| Stx1d (VT1d) mutant A subunit | 21 or 23 | I, II & V or I, III & V |
| Stx2a (VT2a) mutant A subunit | 21 or 23 | I, II & V or I, III & V |
| Stx2b (VT2b) mutant A subunit | 21 or 23 | I, II & V or I, III & V |
| Stx2c (VT2c) mutant A subunit | 21 or 23 | I, II & V or I, III & V |
| Stx2d (VT2d) mutant A subunit | 21 or 23 | I, II & V or I, III & V |
| Stx2e (VT2e) mutant A subunit | 21 or 23 | I, II & V or I, III & V |
| Stx2f (VT2f) mutant A subunit | 21 or 23 | I, II & V or I, III & V |
| Stx2g (VT2g) mutant A subunit | 21 or 23 | I, II & V or I, III & V |
| Heat-labile enterotoxin, | 21 or 23 | I, II & V or I, III & V |
| mutant A subunit | | |
| Heat-labile enterotoxin, | 21 or 23 | I, II & V or I, III & V |
| mutant A subunit (LT-B, porcine) | | |
| Heat-labile enterotoxin IIA, | 21 or 23 | I, II & V or I, III & V |
| mutant A subunit (LT-IIA) | | |
| Heat-labile enterotoxin IIB, | 21 or 23 | I, II & V or I, III & V |
| mutant A subunit (LT-IIB) | | |
| Pertussis toxin, mutant A subunit | 21 or 23 | I, II & V or I, III & V |
| E. coli subtilase cytotoxin, | 21 or 23 | I, II & V or I, III & V |
| mutant A subunit | | |
| AB type: | | |
| Abrin B subunit | 1 | II & V |
| Bodinierin B subunit | 1 | II & V |
| Cinnamomin B subunit | 1 | II & V |
| Modeccin B subunit | 1 | II & V |
| Porrectin B subunit | 1 | II & V |
| Ricin B subunit | 1 | II & V |
| Sambucus ribosome | 1 | II & V |
| inactivating proteins, B subunits | | |
| Viscumin B subunit | 1 | II & V |
| Volkensin B subunit | 1 | II & V |
| Abrin mutant A subunit | 1 | II & V |
| Bodinierin mutant A subunit | 1 | II & V |
| Cinnamomin mutant A subunit | 1 | II & V |
| Modeccin mutant A subunit | 1 | II & V |
| Porrectin mutant A subunit | 1 | II & V |
| Ricin mutant A subunit | 1 | II & V |
| Sambucus ribosome inactivating proteins, | 1 | II & V |
| mutant A subunits | | |
| Viscumin mutant A subunit | 1 | II & V |
| Volkensin mutant A subunit | 1 | II & V |

### Example (26)

### Synthesis of DARE™ Delivery System Delivery Modules and Preparation of the Modules-siRNA Conjugate DARE™-AMF-COX2STEL-siRNA

(i) Synthesis of Module (c)

A module (c) molecule comprising H₂N—C(NPyS)SGSGSG-(DprAoa)SGSGSGNASSSRSG LDDINPTVLLKERSTEL [module (c) comprises SEQ ID NO: 44 (COX2STEL peptide) with dual link positions using two linker peptides each comprising SEQ ID NO: 98] is synthesized commercially by solid-phase Fmoc peptide chemistry. Deprotection is performed in the standard fashion and the crude product is purified by reversed phase HPLC to give a purity >95%. The activated cysteine residue is introduced using Boc-Cys(NPys)-OH (Bachem product no. A-2825) as a building block. Fmoc-Dpr(Boc-Aoa)-OH((N-a-Fmoc-N-β-(N-t.-Boc-aminooxyacetyl)-L-diaminopropionic acid; Novabiochem product no. 04-12-1185) is used to introduce the N-β-aminooxyacetyl L-diaminopropionyl residue. Quality control (QC) of the purified peptide is done by amino acid analysis, electrospray mass spectroscopy (ESMS) and analytical reversed phase HPLC.

(ii) Synthesis of the Delivery Carrier Comprising Functionalized Module [(a)+(b)]+(c)

To continue, 6.27 mg (equivalent to 50 nmol of dimer) of lyophilized rabbit muscle phosphoglucose isomerase (PGI, AMF; from Sigma-Aldrich, product no. P9544) is carefully dissolved in 5 mL of sterile degassed PBS containing 500 μM EDTA, pH 7.4, centrifuged to remove any insoluble material and the supernatant is then desalted and buffer exchanged 5× using a Vivaspin 15R centrifugal concentrator (30 kDa MWCO, Sartorius Stedim Biotech product no. VS15RH21). Initially the protein solution is diluted with 5 mL of the above buffer concentrated to a volume of 1 mL and then washed with 5×9 mL of buffer at room temperature, each time reducing the volume to 1 mL. A solution of 200 nmol (0.79 mg) of module (c), Example 26 (i) dissolved in 0.5 mL of PBS buffer containing 500 μM EDTA, pH 7.4 is then added and the reaction mixture kept for 5 h at room temperature under a nitrogen atmosphere. The desired product is purified by preparative gel filtration [Size Exclusion Chromatography (SEC)] using a HiLoad 16/60 Superdex 200 prep grade column (GE Healthcare, part no. 17-1069-01) eluted with PBS at a flow rate of 1 mL/min. The column effluent is monitored at 280 and 343 nm. Identification of the desired carrier peak is enabled by having previously calibrated the SEC column with AMF and with module (c) Example 26 (i). Product containing fractions are pooled and concentrated to a volume of 1 mL using a Vivaspin 20 protein concentrator (10 kDa MWCO, Sartorius Stedim Biotech product no. VS2001). QC of the delivery carrier is done by mass spectroscopy and by PAGE using silver staining.

(iii) Preparation of the Cargo siRNAs [Compounds (d)]

A double stranded RNA molecule comprised of two 21 mer strands, with a double stranded region of 19 nucleotides in length and 2 nucleotides overhanging at the 3′ end of each strand, and targeting glyceraldehyde 3-phosphate dehydrogenase (GAPDH), wherein the sense strand comprises CCAuCUUCCAGGAGCgAGAuu (SEQ ID NO: 194), wherein lowercase u or g represents a 2′-O-Me-modified nucleotide; and the antisense strand comprises UCUCGCUCCUGgAAGAuGGdTdG (SEQ ID NO: 195), wherein lowercase u or g represents a 2′-O-Me-modified nucleotide and wherein the antisense strand has a 5′-phosphate and deoxynucleotides at its 3′ end (dNdN), is synthesized such that the 5P-terminus of the sense strand is modified with 5′-(C6 aminolinker)-phosphate-(C6-SS—C6 spacer)-phosphate-Cy3. In addition, a double stranded RNA molecule comprised of two 21 mer strands, with a double stranded region of 19 nucleotides in length and 2 nucleotides overhanging at the 3′-end of each strand, and targeting firefly luciferase (fLuc), wherein the sense strand comprises 5′-CUUACgCUGAGuACUUCGAuu (SEQ ID NO: 197), wherein lowercase u or g represents a 2′-O-methylribonucleotide; and the antisense strand comprises 5′-UCGAAGUACUC AgCGUAAgdTdG (SEQ ID NO: 198), wherein lower case “g” represents a 2′-O-methylribonucleotide and wherein the antisense strand has a 5′-phosphate and deoxynucleotides at its 3′-end (dNdN), is synthesized such that the 5′-terminus of the sense strand is modified with a 5-(C6-SS—C6 spacer)-phosphate-Cy3 moiety. The four HPLC-purified individual single strands are all analyzed by HPLC and MALDI-TOF MS. The Cy3 dye is for tracking purposes by fluorescence and the disulfide bond ensures that the cargo can finally be released within the reducing environment of the cell cytoplasm. The single strands are analyzed by ESMS and analytical HPLC for QC prior to annealing. The desalted lyophilized siRNA (200 nmol) is dissolved in 1 mL of sterile 100 mM sodium tetraborate buffer pH 8.5 and reacted with 200 μL, of a fresh solution of 3.49 mg (10 μmol) of sulfosuccinimidyl 4-formylbenzoate (sulfo-S-4FB, SoluLink, product no. S-1008-105) dissolved in 500 μL, of sterile 100 mM sodium tetraborate buffer, pH 8.5 for 3 h at RT. The siRNA bearing a benzaldehyde function is then desalted and buffer exchanged against degassed 100 mM sodium phosphate buffer, 150 mM NaCl, 500 μM EDTA, pH 6 using a Vivaspin 15 centrifugal concentrator (5 kDa MWCO, Sartorius Stedim Biotech product no. VS1511). Initially the benzaldehyde modified siRNA solution is diluted with 8.5 mL of sterile degassed 100 mM sodium phosphate, 150 mM sodium chloride, 500 μM EDTA, pH 6 concentrated to a volume of 1.5 mL and then washed with 5×8.5 mL of sterile degassed 100 mM sodium phosphate, 150 mM sodium chloride, 500 μM EDTA, pH 6 at room temperature, each time reducing the volume to 1.5 mL. QC of the benzaldehyde modified siRNA is done by ESMS and analytical HPLC. A small aliquot of the sample is analyzed for the presence of the aldehyde moiety by reaction with an excess of Cascade Blue hydrazide (Molecular Probes, catalog no. C-687) in buffer at pH 5, desalted by ethanol precipitation and analyzed by native anion-exchange HPLC on a MonoQ column (GE Healthcare) using multiwavelength detection (260 nm for the RNA, 399 nm for the Cascade Blue and 550 nm for the Cy3).

(iv) Coupling of the Cargo [Compound (d)] to the Delivery Carrier [Modules [[(a)+(b)]+(c)]

To prepare the conjugate DARE™-AMF-COX2STEL-siRNA of FIG. 22, the delivery carrier from Example 26 (ii) (max. 25 nmol, based on AMF dimer) in 500 μL, of 100 mM phosphate buffer containing 100 mM aniline pH 7 is mixed with 100 nmol of the linker-siRNA from Example 26(iii) above in 500 μL, of 100 mM phosphate buffer containing 100 mM aniline and kept for 24 h at RT. The desired conjugate is purified by preparative SEC on a HiLoad 16/60 Superdex 200 prep grade column (GE Healthcare, part no. 17-1069-01) eluted at 1 mL/min with sterile PBS, pH 7.4. The column effluent is monitored at 260 nm and 550 nm. Calibration of the column is carried out prior to the preparative purification using the individual reaction components. Those fractions containing the desired conjugate are combined and concentrated to a volume of 1 mL by ultrafiltration using a Vivaspin 20 concentrator (30 kDa MWCO, Sartorius Stedim Biotech product no. VS2021) and the final concentrate is stored at 4° C. This procedure applies to both GAPDH and fLuc. QC of both constructs is performed by mass spectroscopy and by native gel electrophoresis including reaction components as markers. In addition a small aliquot of each construct should be treated with fresh 50 mM DTT solution for 30 min at RT to break each construct into 3 components and analyses by SDS-PAGE should be done with staining by silver (ProteoSilver™ Plus, Sigma) and also by SYPRO® Red using authentic AMF, module (c) and siRNA as markers.

### Example (27)

### Synthesis of DARE™ Delivery System Delivery Modules and Preparation of the Modules-siRNA Conjugate DARE™-AMF-MYCIGMμ-siRNA

(i) Synthesis of Module (c)

A module (c) molecule comprising H₂N—C(NPyS)SGSGSG-(DprAoa)SGSGSGEQKLISEED LGKPTLYQVSLIMSDTGGTSY (module (c) comprises SEQ ID NO: 54 (IgMu), SEQ ID NO: 305 (c-myc epitope tag), and two linker peptides each comprising SEQ ID NO:98 and providing dual link positions) is synthesized commercially by solid-phase Fmoc peptide chemistry. Deprotection is performed in the standard fashion and the crude product is purified by reversed phase HPLC to give a purity >95%. The activated cysteine residue is introduced using Boc-Cys(NPys)-OH (Bachem product no. A-2825) as a building block. Fmoc-Dpr(Boc-Aoa)-OH ((N-α-Fmoc-N-β-(N-t.-Boc-aminooxyacetyl)-L-diaminopropionic acid; Novabiochem product no. 04-12-1185) is used to introduce the N-β-aminooxyacetyl L-diaminopropionyl residue. Quality control (QC) of the purified peptide is done by amino acid analysis, electrospray mass spectroscopy (ESMS) and analytical reversed phase HPLC.

(ii) Synthesis of the Delivery Carrier Comprising Functionalized Module [(a)+(b)]+(c)

To continue, 6.27 mg (equivalent to 50 nmol of dimer) of lyophilized rabbit muscle phosphoglucose isomerase (PGI, AMF; SEQ ID NO: 311; from Sigma-Aldrich, product no. P9544) is carefully dissolved in 5 mL of sterile degassed PBS containing 500 μM EDTA, pH 7.4, centrifuged to remove any insoluble material and the supernatant is then desalted and buffer exchanged 5× using a Vivaspin 15R centrifugal concentrator (30 kDa MWCO, Sartorius Stedim Biotech product no. VS15RH21). Initially the protein solution is diluted with 5 mL of the above buffer concentrated to a volume of 1 mL and then washed with 5×9 mL of buffer at room temperature, each time reducing the volume to 1 mL. A solution of 200 nmol (0.79 mg) of module (c), Example 27 (i) dissolved in 0.5 mL of PBS buffer containing 500 μM EDTA, pH 7.4 is then added and the reaction mixture kept for 5 h at room temperature under a nitrogen atmosphere. The desired product is purified by preparative gel filtration [Size Exclusion Chromatography (SEC)] using a HiLoad 16/60 Superdex 200 prep grade column (GE Healthcare, part no. 17-1069-01) eluted with PBS at a flow rate of 1 mL/min. The column effluent is monitored at 280 and 343 nm. Identification of the desired carrier peak is enabled by having previously calibrated the SEC column with AMF [(a)+(b)] and with module (c) Example 27 (i). Product containing fractions are pooled and concentrated to a volume of 1 mL using a Vivaspin 20 protein concentrator (10 kDa MWCO, Sartorius Stedim Biotech product no. VS2001). QC of the delivery carrier is done by mass spectroscopy and by PAGE using silver staining

(iii) Preparation of the Cargo siRNAs [Compounds (d)]

A double stranded RNA molecule comprised of two 21 mer strands, with a double stranded region of 19 nucleotides in length and 2 nucleotides overhanging at the 3′ end of each strand, and targeting glyceraldehyde 3-phosphate dehydrogenase (GAPDH), wherein the sense strand comprises CCAuCUUCCAGGAGCgAGAuu (SEQ ID NO: 194), wherein lowercase u or g represents a 2′-O-Me-modified nucleotide; and the antisense strand comprises UCUCGCUCCUGgAAGAuGGdTdG (SEQ ID NO: 195), wherein lowercase u or g represents a 2′-O-Me-modified nucleotide and wherein the antisense strand has a 5′-phosphate and deoxynucleotides at its 3′ end (dNdN), is synthesized such that the 5P-terminus of the sense strand is modified with 5′-(C6 aminolinker)-phosphate-(C6-SS—C6 spacer)-phosphate-Cy3. In addition, a double stranded RNA molecule comprised of two 21 mer strands, with a double stranded region of 19 nucleotides in length and 2 nucleotides overhanging at the 3′-end of each strand, and targeting firefly luciferase (fLuc), wherein the sense strand comprises 5′-CUUACgCUGAGuACUUCGAuu (SEQ ID NO: 197), wherein lowercase u or g represents a 2′-O-methylribonucleotide; and the antisense strand comprises 5′-UCGAAGUACUC AgCGUAAgdTdG (SEQ ID NO: 198), wherein lowercase g represents a 2′-O-methylribonucleotide and wherein the antisense strand has a 5′-phosphate and deoxynucleotides at its 3′-end (dNdN), is synthesized such that the 5′-terminus of the sense strand is modified with a 5-(C6-SS—C6 spacer)-phosphate-Cy3 moiety. The four HPLC-purified individual single strands are all analyzed by HPLC and MALDI-TOF MS. The Cy3 dye is for tracking purposes by fluorescence and the disulfide bond ensures that the cargo can finally be released within the reducing environment of the cell cytoplasm. The single strands are analyzed by ESMS and analytical HPLC for QC prior to annealing. The desalted lyophilized siRNA (200 nmol) is dissolved in 1 mL of sterile 100 mM sodium tetraborate buffer pH 8.5 and reacted with 200 μL of a fresh solution of 3.49 mg (10 mmol) of sulfosuccinimidyl 4-formylbenzoate (sulfo-S-4FB, SoluLink, product no. S-1008-105) dissolved in 500 μL of sterile 100 mM sodium tetraborate buffer, pH 8.5 for 3 h at RT. The siRNA bearing a benzaldehyde function is then desalted and buffer exchanged against degassed 100 mM sodium phosphate buffer, 150 mM NaCl, 500 μM EDTA, pH 6 using a Vivaspin 15 centrifugal concentrator (5 kDa MWCO, Sartorius Stedim Biotech product no. VS1511). Initially the benzaldehyde modified siRNA solution is diluted with 8.5 mL of sterile degassed 100 mM sodium phosphate, 150 mM sodium chloride, 500 μM EDTA, pH 6 concentrated to a volume of 1.5 mL and then washed with 5×8.5 mL of sterile degassed 100 mM sodium phosphate, 150 mM sodium chloride, 500 μM EDTA, pH 6 at room temperature, each time reducing the volume to 1.5 mL. QC of the benzaldehyde modified siRNA is done by ESMS and analytical HPLC. A small aliquot of the sample is analyzed for the presence of the aldehyde moiety by reaction with an excess of Cascade Blue hydrazide (Molecular Probes, catalog no. C-687) in buffer at pH 5, desalted by ethanol precipitation and analyzed by native anion-exchange HPLC on a MonoQ column (GE Healthcare) using multiwavelength detection (260 nm for the RNA, 399 nm for the Cascade Blue and 550 nm for the Cy3).

(iv) Coupling of the Cargo [Compound (d)] to the Delivery Carrier [Modules [(a)+(b)]+(c)]

To prepare the conjugate DARE™-AMF-MYCIGKit-siRNA of FIG. 23, the delivery carrier from Example 27 (ii) (max. 25 nmol, based on AMF dimer) in 500 μL of 100 mM phosphate buffer containing 100 mM aniline pH 7 is mixed with 100 nmol of the linker-siRNA from Example 27 (iii) above in 500 μL of 100 mM phosphate buffer containing 100 mM aniline and kept for 24 h at RT. The desired conjugate is purified by preparative SEC on a HiLoad 16/60 Superdex 200 prep grade column (GE Healthcare, part no. 17-1069-01) eluted at 1 mL/min with sterile PBS, pH 7.4. The column effluent is monitored at 260 nm and 550 nm. Calibration of the column is carried out prior to the preparative purification using the individual reaction components. Those fractions containing the desired conjugate are combined and concentrated to a volume of 1 mL by ultrafiltration using a Vivaspin 20 concentrator (30 kDa MWCO, Sartorius Stedim Biotech product no. VS2021) and the final concentrate is stored at 4° C. This procedure applies to both GAPDH and fLuc. QC of both constructs is performed by mass spectroscopy and by native gel electrophoresis including reaction components as markers. In addition a small aliquot of each construct should be treated with fresh 50 mM DTT solution for 30 min at RT to break each construct into 3 components and analyses by SDS-PAGE should be done with staining by silver (ProteoSilver™ Plus, Sigma) and also by SYPRO® Red using authentic AMF, module (c) and siRNA as markers.

### Example (28)

### Synthesis of DARE™ Delivery System Delivery Modules and Preparation of the Modules-siRNA Conjugate DARE™-CTB-COX2STEL-siRNA

(i) Synthesis of Module (c)

A module (c) molecule: H₂N—C(NPyS)SGSGSG-(DprAoa)SGSGSGNASSSR SGLDDINPTVLLKERSTEL [module (c) comprises SEQ ID NO: 44 (COX2STEL peptide) with dual link positions using two linker peptides each comprising SEQ ID NO: 98] is synthesized commercially by solid-phase Fmoc peptide chemistry. Deprotection is performed in the standard fashion and the crude product is purified by reversed phase HPLC to give a purity >95%. The activated cysteine residue is introduced using Boc-Cys(NPys)-OH (Bachem product no. A-2825) as a building block. Fmoc-Dpr(Boc-Aoa)-OH((N-a-Fmoc-N-β-(N-t.-Boc-aminooxyacetyl)-L-diaminopropionic acid; Novabiochem product no. 04-12-1185) is used to introduce the N-β-aminooxyacetyl L-diaminopropionyl residue. Quality control (QC) of the purified peptide is done by amino acid analysis, electrospray mass spectroscopy (ESMS) and analytical reversed phase HPLC. To activate module (c), 600 nmol of (i) is transferred to an Eppendorf vial and treated for 1 h with 15 mg/mL DTT after which it is purified by preparative gel filtration [Size Exclusion Chromatography (SEC)] using a HiLoad™ 16/60 Superdex™ 75 prep grade column (GE Healthcare, part no. 17-1068-01) eluted with PBS (degassed with N₂) at a flow rate of 1 mL/min. Identification of the desired carrier peak is enabled by having calibrated the SEC column with module (c). Product containing fractions are pooled and lyophilised. Deprotected module (c) is employed immediately in step (iii). Alternatively module (c) is treated for 1 h with 15 mg/mL DTT and transferred to a PD Miditrap™ G-10 (GE Healthcare 28-9180-11) pre-equilibrated with PBS, 250 nM EDTA. The fraction containing the deprotected module (c) is employed immediately in step (iii).

(ii) Functionalization of Module [(a)+(b)]:

To functionalize module [(a)+(b)], 23.2 mg (400 nmol of pentamer) of recombinant cholera toxin subunit B [(CTB; SEQ ID NO: 117), obtained from SBL Vaccin AB, Matfors, Sweden, see www.rctb.net/theproduct.htm] in 0.5 mL of sterile PBS is mixed with a fresh solution of 2.1 mg (4 mmol) of sulfosuccinimidyl 6-[3′-(2-pyridyldithio)-propionamido] hexanoate (sulfo-LC-SPDP) in 300 μL of sterile PBS pH 7.4 in a sterile 1 mL Eppendorf tube and kept 1.5 h at room temperature with occasional mixing. The solution is then desalted and buffer exchanged against degassed 10 mM sodium phosphate buffer, 150 mM NaCl, pH 7.4 (PBS) containing 250 nM EDTA using a Vivaspin 6 centrifugal concentrator (10 kDa MWCO, Sartorius Stedim Biotech product no. VS0601). Initially the functionalized CTB solution is diluted with 5 mL of PBS plus 250 μM EDTA concentrated to a volume of 0.5 mL and then washed with 6×5 mL of PBS plus 250 μM EDTA at room temperature, each time reducing the volume to 0.5 mL. In order to determine the 2-pyridyl-disulfide loading of the functionalized CTB a small aliquot (10 μL) of the solution is diluted to 1 mL with PBS and the absorbance at 343 nm measured using a quartz cuvette with a 1 cm pathlength. A 10 μL aliquot of a 15 mg/mL solution of dithiothreitol (DTT) in PBS is added to the cuvette, the contents are mixed carefully and the absorbance at 343 nm is measured after 15 min at room temperature, enabling the amount of pyridine-2-thione released to be quantitated (ε₃₄₃ₙₘ is 8080 M⁻¹ cm⁻¹, i.e. 1 μmol is equivalent to 8.08 A₃₄₃ units). The 2-pyridyl-disulfide loading of the CTB is generally around 5 μmol per μmol of pentamer. It is clear to one skilled in the art that the loading can be reduced by reducing the excess of sulfo-LC-SPDP used in the functionalization.

(iii) Synthesis of the Delivery Carrier Comprising Functionalized Module [(a)+(b)]+(c)

250 μL of solution containing functionalized CTB (50 nmol), [(a)+(b)] from Example 28(ii) above is reacted for 18 h at RT under nitrogen with 1.0 mg (250 nmol) of the linkage molecule containing module (c) from Example 28(i) above dissolved in 750 μL of 100 mM phosphate buffer pH 7.4. Following a brief centrifugation the desired carrier [modules [(a)+(b)]+(c)] is then purified by preparative gel filtration [Size Exclusion Chromatography (SEC)] using a HiLoad™ 16/60 Superdex™ 75 prep grade column (GE Healthcare, part no. 17-1068-01) eluted with PBS at a flow rate of 1 mL/min. Identification of the desired carrier peak is enabled by having calibrated the SEC column with CTB and with the linker-peptide entity from Example 28(i). Product containing fractions are pooled and concentrated to a volume of 0.5 mL using a Vivaspin 20 protein concentrator (10 kDa MWCO, Sartorius Stedim Biotech product no. VS2001).

(iv)Preparation of the Cargo siRNAs [Module d]

A double stranded RNA molecule comprised of two 21 mer strands, with a double stranded region of 19 nucleotides in length and 2 nucleotides overhanging at the 3′ end of each strand, and targeting glyceraldehyde 3-phosphate dehydrogenase (GAPDH), wherein the sense strand comprises CCAuCUUCCAGGAGCgAGAuu (SEQ ID NO: 194), wherein lowercase u or g represents a 2′-O-Me-modified nucleotide; and the antisense strand comprises UCUCGCUCCUGgAAGAuGGdTdG (SEQ ID NO: 195), wherein lowercase u or g represents a 2′-O-Me-modified nucleotide and wherein the antisense strand has a 5′-phosphate and deoxynucleotides at its 3′ end (dNdN), is synthesized such that the 5′-terminus of the sense strand is modified with 5′-(C6 aminolinker)-phosphate-(C6-SS—C6 spacer)-phosphate-Cy3. In addition, a double stranded RNA molecule comprised of two 21 mer strands, with a double stranded region of 19 nucleotides in length and 2 nucleotides overhanging at the 3′-end of each strand, and targeting firefly luciferase (fLuc), wherein the sense strand comprises 5′-CUUACgCUGAGuACUUCGAuu (SEQ ID NO: 197), wherein lowercase u or g represents a 2′-O-methylribonucleotide; and the antisense strand comprises 5′-UCGAAGUACUC AgCGUAAgdTdG (SEQ ID NO: 198), wherein lowercase g represents a 2′-β-methylribonucleotide and wherein the antisense strand has a 5′-phosphate and deoxynucleotides at its 3′-end (dNdN), is synthesized such that the 5′-terminus of the sense strand is modified with a 5-(C6-SS—C6 spacer)-phosphate-Cy3 moiety. The four HPLC-purified individual single strands are all analyzed by HPLC and MALDI-TOF MS. The Cy3 dye is for tracking purposes by fluorescence and the disulfide bond ensures that the cargo can finally be released within the reducing environment of the cell cytoplasm. The single strands are analyzed by ESMS and analytical HPLC for QC prior to annealing. The desalted lyophilized siRNA (200 nmol) is dissolved in 1 mL of sterile 100 mM sodium tetraborate buffer pH 8.5 and reacted with 200 μL of a fresh solution of 3.49 mg (10 mmol) of sulfosuccinimidyl 4-formylbenzoate (sulfo-S-4FB, SoluLink, product no. S-1008-105) dissolved in 500 μL of sterile 100 mM sodium tetraborate buffer, pH 8.5 for 3 h at RT. The siRNA bearing a benzaldehyde function is then desalted and buffer exchanged against degassed 100 mM sodium phosphate buffer, 150 mM NaCl, 500 μM EDTA, pH 6 using a Vivaspin 15 centrifugal concentrator (5 kDa MWCO, Sartorius Stedim Biotech product no. VS1511). Initially the benzaldehyde modified siRNA solution is diluted with 8.5 mL of sterile degassed 100 mM sodium phosphate, 150 mM sodium chloride, 500 μM EDTA, pH 6 concentrated to a volume of 1.5 mL and then washed with 5×8.5 mL of sterile degassed 100 mM sodium phosphate, 150 mM sodium chloride, 500 μM EDTA, pH 6 at room temperature, each time reducing the volume to 1.5 mL. QC of the benzaldehyde modified siRNA is done by ESMS and analytical HPLC. A small aliquot of the sample is analyzed for the presence of the aldehyde moiety by reaction with an excess of Cascade Blue hydrazide (Molecular Probes, catalog no. C-687) in buffer at pH 5, desalted by ethanol precipitation and analyzed by native anion-exchange HPLC on a MonoQ column (GE Healthcare) using multiwavelength detection (260 nm for the RNA, 399 nm for the Cascade Blue and 550 nm for the Cy3). This procedure applies to both GAPDH and fLuciferase.

(v) Coupling of the Cargo [Compound d] to the Delivery Carrier [Modules [(a)+(b)]+(c)]

To prepare the delivery conjugate of FIG. 24, the delivery carrier (40 nmol, based on CTB pentamer) from Example 28 (iii) above in 500 μL of 100 mM phosphate buffer containing 150 mM NaCl, 100 mM aniline pH 7 is mixed with 200 nmol of the adapter-siRNA component (cargo) from Example 28(iv) above in 500 μL of 100 mM phosphate buffer containing 150 mM NaCl, 100 mM aniline and kept for 48 h at RT. The desired conjugate is purified by preparative SEC on a HiLoad™ 16/60 Superdex™ 200 prep grade column (GE Healthcare, part no. 17-1069-01) eluted at 1 mL/min with sterile PBS, pH 7.4. The column effluent is monitored at 260 nm and 550 nm. Calibration of the column is carried out prior to the preparative purification using the individual reaction components. Those fractions containing the desired conjugate are combined and concentrated by ultrafiltration using a Vivaspin 20 concentrator (30 kDa MWCO, Sartorius Stedim Biotech product no. VS2021) and the final concentrate is stored at 4° C. QC is performed by native gel electrophoresis and analytical SEC on a Superdex™ 200 10/300 GL column (GE Healthcare, part no. 17-5175-01). This procedure applies to both GAPDH and fLuciferase.

One of skill in the art may exploit the c-myc epitope used in this Example for purification and/or intracellular detection and localization of the resulting c-myc-tagged module (c) comprising delivery conjugate preferably through the use of a mouse anti-c-myc 1-9e10 antibody (Roche, catalog #11667149001) according to standard methods (see also Frieden et al., 2004. Chem. BioDivers., 1:930-938. and Gottschling et al., 1998. Bioconjugate Chem., 9: 831-837.).

### Example (29)

### Synthesis of DARE™ Delivery System Delivery Modules and Preparation of the Modules-siRNA Conjugate DARE™-CTB-MYCIGMu-siRNA

(i) Synthesis of Module (c)

A module (c) molecule: H₂N—C(NPyS)SGSGSG-(DprAoa)SGSGSGGEQKLISEEDL GKPTLYQVSLIMSDTGGTSY (module (c) comprises SEQ ID NO: 54 (IgMO, SEQ ID NO: 305 (c-myc epitope tag), and two linker peptides each comprising SEQ ID NO:98 and providing dual link positions) is synthesized commercially by solid-phase Fmoc peptide chemistry. Deprotection is performed in the standard fashion and the crude product is purified by reversed phase HPLC to give a purity >95%. The activated cysteine residue is introduced using Boc-Cys(NPys)-OH (Bachem product no. A-2825) as a building block. Fmoc-Dpr(Boc-Aoa)-OH ((N-α-Fmoc-N-β-(N-t.-Boc-aminooxyacetyl)-L-diaminopropionie acid; Novabiochem product no. 04-12-1185) is used to introduce the N-β-aminooxyacetyl L-diaminopropionyl residue. Quality control (QC) of the purified peptide is done by amino acid analysis, electrospray mass spectroscopy (ESMS) and analytical reversed phase HPLC. To activate module (c), 600 nmol of (i) was transferred to an Eppendorf vial and treated for 1 h with 15 mg/mL DTT after which it is purified by preparative gel filtration [Size Exclusion Chromatography (SEC)] using a HiLoad™ 16/60 Superdex™ 75 prep grade column (GE Healthcare, part no. 17-1068-01) eluted with PBS (degassed with N₂) at a flow rate of 1 mL/min. Identification of the desired carrier peak is enabled by having calibrated the SEC column with module (c). Product containing fractions are pooled and lyophilised. Deprotected module (c) is employed immediately in step (iii). Alternatively module (c) was treated for 1 hour with 15 mg/mL DTT and transferred to a PD Miditrap™ G-10 (GE Healthcare 28-9180-11) pre-equilibrated with PBS, 250 uM EDTA. The fraction containing the deprotected module (c) was employed immediately in step (iii).

(ii) Functionalization of Module [(a)+(b)]:

To functionalize module [(a)+(b)], 23.2 mg (400 nmol of pentamer) of recombinant cholera toxin subunit B [(CTB; SEQ ID NO: 117), obtained from SBL Vaccin AB, Matfors, Sweden, see www.rctb.net/theproduct.htm] in 0.5 mL of sterile PBS is mixed with a fresh solution of 2.1 mg (4 μmol) of sulfosuccinimidyl 6-[3′-(2-pyridyldithio)-propionamido] hexanoate (sulfo-LC-SPDP) in 300 μL of sterile PBS pH 7.4 in a sterile 1 mL Eppendorf tube and kept 1.5 h at room temperature with occasional mixing. The solution is then desalted and buffer exchanged against degassed 10 mM sodium phosphate buffer, 150 mM NaCl, pH 7.4 (PBS) containing 250 μM EDTA using a Vivaspin 6 centrifugal concentrator (10 kDa MWCO, Sartorius Stedim Biotech product no. VS0601). Initially the functionalized CTB solution is diluted with 5 mL of PBS plus 250 μM EDTA concentrated to a volume of 0.5 mL and then washed with 6×5 mL of PBS plus 250 μM EDTA at room temperature, each time reducing the volume to 0.5 mL. In order to determine the 2-pyridyl-disulfide loading of the functionalized CTB a small aliquot (10 μL) of the solution is diluted to 1 mL with PBS and the absorbance at 343 nm measured using a quartz cuvette with a 1 cm pathlength. A 10 μL aliquot of a 15 mg/mL solution of dithiothreitol (DTT) in PBS is added to the cuvette, the contents are mixed carefully and the absorbance at 343 nm is measured after 15 min at room temperature, enabling the amount of pyridine-2-thione released to be quantitated (ε₃₄₃ₙₘ is 8080 M⁻¹ cm⁻¹, i.e. 1 μmol is equivalent to 8.08 A₃₄₃ units). The 2-pyridyl-disulfide loading of the CTB is generally around 5 μmol per μmol of pentamer. It is clear to one skilled in the art that the loading can be reduced by reducing the excess of sulfo-LC-SPDP used in the functionalization.

(iii) Synthesis of the Delivery Carrier Comprising Functionalized Module [(a)+(b)]+(c)

250 μL of solution containing functionalized CTB [(a)+(b)] (50 nmol) from Example 29(ii) above is reacted for 18 h at RT under nitrogen with 1.1 mg (250 nmol) of the linkage molecule containing module (c) from Example 29(i) above dissolved in 750 μL of 100 mM phosphate buffer pH 7.4. Following a brief centrifugation the desired carrier [modules [(a)+(b)]+(c)] is then purified by preparative gel filtration [Size Exclusion Chromatography (SEC)] using a HiLoad™ 16/60 Superdex™ 75 prep grade column (GE Healthcare, part no. 17-1068-01) eluted with PBS at a flow rate of 1 mL/min. Identification of the desired carrier peak is enabled by having calibrated the SEC column with CTB and with the linker-peptide entity from Example 29(i). Product containing fractions are pooled and concentrated to a volume of 0.5 mL using a Vivaspin 20 protein concentrator (10 kDa MWCO, Sartorius Stedim Biotech product no. VS2001).

(iv) Preparation of the Cargo siRNAs [Module d]

A double stranded RNA molecule comprised of two 21 mer strands, with a double stranded region of 19 nucleotides in length and 2 nucleotides overhanging at the 3′ end of each strand, and targeting glyceraldehyde 3-phosphate dehydrogenase (GAPDH), wherein the sense strand comprises CCAuCUUCCAGGAGCgAGAuu (SEQ ID NO: 194), wherein lowercase u or g represents a 2′-O-Me-modified nucleotide; and the antisense strand comprises UCUCGCUCCUGgAAGAuGGdTdG (SEQ ID NO: 195), wherein lowercase u or g represents a 2′-O-Me-modified nucleotide and wherein the antisense strand has a 5′-phosphate and deoxynucleotides at its 3′ end (dNdN), is synthesized such that the 5P-terminus of the sense strand is modified with 5′-(C6 aminolinker)-phosphate-(C6-SS—C6 spacer)-phosphate-Cy3. In addition, a double stranded RNA molecule comprised of two 21 mer strands, with a double stranded region of 19 nucleotides in length and 2 nucleotides overhanging at the 3′-end of each strand, and targeting firefly luciferase (fLuc), wherein the sense strand comprises 5′-CUUACgCUGAGuACUUCGAuu (SEQ ID NO: 197), wherein lowercase u or g represents a 2′-O-methylribonucleotide; and the antisense strand comprises 5′-UCGAAGUACUC AgCGUAAgdTdG (SEQ ID NO: 198), wherein lowercase g represents a 2′-O-methylribonucleotide and wherein the antisense strand has a 5′-phosphate and deoxynucleotides at its 3′-end (dNdN), is synthesized such that the 5′-terminus of the sense strand is modified with a 5-(C6-SS—C6 spacer)-phosphate-Cy3 moiety. The four HPLC-purified individual single strands are all analyzed by HPLC and MALDI-TOF MS. The Cy3 dye is for tracking purposes by fluorescence and the disulfide bond ensures that the cargo can finally be released within the reducing environment of the cell cytoplasm. The single strands are analyzed by ESMS and analytical HPLC for QC prior to annealing. The desalted lyophilized siRNA (200 nmol) is dissolved in 1 mL of sterile 100 mM sodium tetraborate buffer pH 8.5 and reacted with 200 μL of a fresh solution of 3.49 mg (10 mmol) of sulfosuccinimidyl 4-formylbenzoate (sulfo-S-4FB, SoluLink, product no. S-1008-105) dissolved in 500 μL of sterile 100 mM sodium tetraborate buffer, pH 8.5 for 3 h at RT. The siRNA bearing a benzaldehyde function is then desalted and buffer exchanged against degassed 100 mM sodium phosphate buffer, 150 mM NaCl, 500 μM EDTA, pH 6 using a Vivaspin 15 centrifugal concentrator (5 kDa MWCO, Sartorius Stedim Biotech product no. VS1511). Initially the benzaldehyde modified siRNA solution is diluted with 8.5 mL of sterile degassed 100 mM sodium phosphate, 150 mM sodium chloride, 500 μM EDTA, pH 6 concentrated to a volume of 1.5 mL and then washed with 5×8.5 mL of sterile degassed 100 mM sodium phosphate, 150 mM sodium chloride, 500 μM EDTA, pH 6 at room temperature, each time reducing the volume to 1.5 mL. QC of the benzaldehyde modified siRNA is done by ESMS and analytical HPLC. A small aliquot of the sample is analyzed for the presence of the aldehyde moiety by reaction with an excess of Cascade Blue hydrazide (Molecular Probes, catalog no. C-687) in buffer at pH 5, desalted by ethanol precipitation and analyzed by native anion-exchange HPLC on a MonoQ column (GE Healthcare) using multiwavelength detection (260 nm for the RNA, 399 nm for the Cascade Blue and 550 nm for the Cy3). This procedure applies to both GAPDH and fLuciferase.

(v) Coupling of the Cargo [Compound d] to the Delivery Carrier [Modules [(a)+(b)]+(c)]

To prepare the delivery conjugate as shown in FIG. 25, the delivery carrier (40 nmol, based on CTB pentamer) from Example 29 (iii) above in 500 μL of 100 mM phosphate buffer containing 150 mM NaCl, 100 mM aniline pH 7 is mixed with 200 nmol of the adapter-siRNA component (cargo) from Example 29(iv) above in 500 μL of 100 mM phosphate buffer containing 150 mM

NaCl, 100 mM aniline and kept for 48 h at RT. The desired conjugate is purified by preparative SEC on a HiLoad™ 16/60 Superdex™ 200 prep grade column (GE Healthcare, part no. 17-1069-01) eluted at 1 mL/min with sterile PBS, pH 7.4. The column effluent is monitored at 260 nm and 550 nm. Calibration of the column is carried out prior to the preparative purification using the individual reaction components. Those fractions containing the desired conjugate are combined and concentrated by ultrafiltration using a Vivaspin 20 concentrator (30 kDa MWCO, Sartorius Stedim Biotech product no. VS2021) and the final concentrate is stored at 4° C. QC is performed by native gel electrophoresis and analytical SEC on a Superdex™ 200 10/300 GL column (GE Healthcare, part no. 17-5175-01). This procedure applies to both GAPDH and fLuciferase.

### Example (30)

### Synthesis of DARE™ Delivery System Delivery Modules and Preparation of the Modules-siRNA Conjugate DARE™-CTB+COX2STEL)(-siRNA)

(i) Synthesis and Activation of Module (c)

A module (c) molecule: H₂N—C(NPyS)SGSGSG-(DprAoa)SGSGSGNASSSR SGLDDINPTVLLKERSTEL [module (c) comprises SEQ ID NO: 44 (COX2STEL peptide) with dual link positions using two linker peptides each comprising SEQ ID NO: 98] is synthesized commercially by solid-phase Fmoc peptide chemistry. Deprotection is performed in the standard fashion and the crude product is purified by reversed phase HPLC to give a purity >95%. The activated cysteine residue is introduced using Boc-Cys(NPys)-OH (Bachem product no. A-2825) as a building block. Fmoc-Dpr(Boc-Aoa)-OH((N-α-Fmoc-N-β-(N-t.-Boc-aminooxyacetyl)-L-diaminopropionic acid; Novabiochem product no. 04-12-1185) is used to introduce the N-β-aminooxyacetyl L-diaminopropionyl residue. Quality control (QC) of the purified peptide is done by amino acid analysis, electrospray mass spectroscopy (ESMS) and analytical reversed phase HPLC. To activate module (c), 600 nmol of (i) is transferred to an Eppendorf vial and treated for 1 h with 15 mg/mL DTT after which it is purified by preparative gel filtration [Size Exclusion Chromatography (SEC)] using a HiLoad™ 16/60 Superdex™ 75 prep grade column (GE Healthcare, part no. 17-1068-01) eluted with PBS (degassed with N₂) at a flow rate of 1 mL/min. Identification of the desired carrier peak is enabled by having calibrated the SEC column with module (c). Product containing fractions are pooled and lyophilised. Deprotected module (c) is employed immediately in step (iii). Alternatively module (c) is treated for 1 h with 15 mg/mL DTT and transferred to a PD Miditrap™ G-10 (GE Healthcare 28-9180-11) pre-equilibrated with PBS, 250 μM EDTA. The fraction containing the deprotected module (c) is employed immediately in step (iii).

(ii) Functionalization of Module [(a)+(b)]:

To functionalize module [(a)+(b)], 5.8 mg (100 nmol of pentamer) of recombinant cholera toxin subunit B [(CTB; SEQ ID NO: 117), obtained from SBL Vaccin AB, Matfors, Sweden, see www.rctb.net/theproduct.htm] in 2.1 mL of sterile PBS is mixed with a fresh solution of 0.26 mg (0.5 mmol) of sulfosuccinimidyl 6-[3′-(2-pyridyldithio)-propionamido] hexanoate (sulfo-LC-SPDP) in 75 μL, of sterile PBS pH 7.4 in a sterile 1 mL Eppendorf tube and kept for 0.5 h at room temperature with occasional mixing. The solution is then desalted and buffer exchanged against degassed 10 mM sodium phosphate buffer, 150 mM NaCl, pH 7.4 (PBS) containing 250 μM EDTA using a Vivaspin 6 centrifugal concentrator (10 kDa MWCO, Sartorius Stedim Biotech product no. VS0601). Initially the functionalized CTB solution is diluted with 5 mL of PBS plus 250 μM EDTA concentrated to a volume of 0.5 mL and then washed with 6×5 mL of PBS plus 250 μM EDTA at room temperature, each time reducing the volume to 0.5 mL. In order to determine the 2-pyridyl-disulfide loading of the functionalized CTB a small aliquot (10 μL) of the solution is diluted to 1 mL with PBS and the absorbance at 343 nm measured using a quartz cuvette with a 1 cm pathlength. A 10 μL aliquot of a 15 mg/mL solution of dithiothreitol (DTT) in PBS is added to the cuvette, the contents are mixed carefully and the absorbance at 343 nm is measured after 15 min at room temperature, enabling the amount of pyridine-2-thione released to be quantitated (ε₃₄₃ₙₘ is 8080 M⁻¹ cm⁻¹, i.e. 1 mmol is equivalent to 8.08 A₃₄₃ units). The 2-pyridyl-disulfide loading of the CTB is generally around 5 mmol per nmol of pentamer.

(iii) Synthesis of the Delivery Carrier Comprising Functionalized Module [(a)+(b)]+(c)

250 μL of solution containing functionalized CTB (50 nmol) from Example 30(ii) above is reacted for 18 h at RT under nitrogen with 1.44 mg (250 nmol) activated module (c) from Example 30(i) dissolved in 750 μL of 100 mM phosphate buffer pH 7.4. Following a brief centrifugation the desired carrier [modules [(a)+(b)]+(c)] is then purified by preparative gel filtration [Size Exclusion Chromatography (SEC)] using a HiLoad™ 16/60 Superdex™ 75 prep grade column (GE Healthcare, part no. 17-1068-01) eluted with PBS at a flow rate of 1 mL/min. Identification of the desired carrier peak is enabled by having calibrated the SEC column with CTB and with module (c) from Example 30(i). Product containing fractions are pooled and concentrated to a volume of 0.5 mL using a Vivaspin 20 protein concentrator (10 kDa MWCO, Sartorius Stedim Biotech product no. VS2001).

(iv) Preparation and Activation of the Cargo siRNAs [Compounds d]

A double stranded RNA molecule comprised of two 21 mer strands, with a double stranded region of 19 nucleotides in length and 2 nucleotides overhanging at the 3′ end of each strand, and targeting glyceraldehyde 3-phosphate dehydrogenase (GAPDH), wherein the sense strand comprises CCAuCUUCCAGGAGCgAGAuu (SEQ ID NO: 194), wherein lowercase u or g represents a 2′-O-Me-modified nucleotide; and the antisense strand comprises UCUCGCUCCUGgAAGAuGGdTdG (SEQ ID NO: 195), wherein lowercase u or g represents a 2′-O-Me-modified nucleotide and wherein the antisense strand has a 5′-phosphate and deoxynucleotides at its 3′ end (dNdN), is synthesized such that the 5P-terminus of the sense strand is modified with 5′-(C6 aminolinker)-phosphate-(C6-SS—C6 spacer)-phosphate-Cy3. In addition, a double stranded RNA molecule comprised of two 21 mer strands, with a double stranded region of 19 nucleotides in length and 2 nucleotides overhanging at the 3′-end of each strand, and targeting firefly luciferase (fLuc), wherein the sense strand comprises 5′-CUUACgCUGAGuACUUCGAuu (SEQ ID NO: 197), wherein lowercase u or g represents a 2′-O-methylribonucleotide; and the antisense strand comprises 5′-UCGAAGUACUC AgCGUAAgdTdG (SEQ ID NO: 198), wherein lowercase g represents a 2′-O-methylribonucleotide and wherein the antisense strand has a 5′-phosphate and deoxynucleotides at its 3′-end (dNdN), is synthesized such that the 5′-terminus of the sense strand is modified with a 5-(C6-SS—C6 spacer)-phosphate-Cy3 moiety. The four HPLC-purified individual single strands are all analyzed by HPLC and MALDI-TOF MS. The Cy3 dye is for tracking purposes by fluorescence and the disulfide bond ensures that the cargo can finally be released within the reducing environment of the cell cytoplasm. The single strands are analyzed by ESMS and analytical HPLC for QC prior to annealing. The desalted lyophilized siRNA (200 nmol) is transferred to an Eppendorf vial and treated for 1 h with 15 mg/mL DTT. The deprotected siRNA is then desalted and buffer exchanged against degassed PBS, 250 μM EDTA using a Vivaspin 15 centrifugal concentrator (5 kDa MWCO, Sartorius Stedim Biotech product no. VS1511). Initially the activated siRNA solution is diluted with 5.0 mL of sterile degassed, 250 μM EDTA, concentrated to a volume of 1.5 mL and then washed with 5×5.0 mL (or until no apparent DTT odor can be smelled anymore) of sterile degassed 250 μM EDTA in PBS, each time reducing the volume to 1.5 mL. The deprotected siRNA is employed immediately in (vi), Example 30. This procedure applies to both GAPDH and fLuciferase siRNAs.

(v) Functionalization of the Delivery Carrier Comprising Module [[(a)+(b)]+(c)]

To functionalize module [[(a)+(b)]+(c)], 50 nmol of module [[(a)+(b)]+(c)], Example 30 (iii), in 0.50 mL of sterile PBS is mixed with 37.5 μL of a fresh stock solution of 0.26 mg (0.5 mmol) of sulfosuccinimidyl 6-[3′-(2-pyridyldithio)-propionamido] hexanoate (sulfo-LC-SPDP) in 75 μL of sterile PBS pH 7.4 in a sterile 1 mL Eppendorf tube and kept 1.5 h at room temperature with occasional mixing. The solution is then desalted and buffer exchanged against degassed 10 mM sodium phosphate buffer, 150 mM NaCl, pH 7.4 (PBS) containing 250 μM EDTA using a Vivaspin 6 centrifugal concentrator (10 kDa MWCO, Sartorius Stedim Biotech product no. VS0601). Initially the functionalized module [[(a)+(b)]+(c)] solution is diluted with 5 mL of PBS plus 250 μM EDTA concentrated to a volume of 0.5 mL and then washed with 6×5 mL of PBS plus 250 μM EDTA at room temperature, each time reducing the volume to 0.5 mL. In order to determine the 2-pyridyl-disulfide loading of the functionalized CTB a small aliquot (10 μL) of the solution is diluted to 1 mL with PBS and the absorbance at 343 nm measured using a quartz cuvette with a 1 cm pathlength. A 10 μL aliquot of a 15 mg/mL solution of dithiothreitol (DTT) in PBS is added to the cuvette, the contents are mixed carefully and the absorbance at 343 nm is measured after 15 min at room temperature, enabling the amount of pyridine-2-thione released to be quantitated (ε₃₄₃ₙₘ is 8080 M⁻¹ cm⁻¹, i.e. 1 mmol is equivalent to 8.08 A₃₄₃ units).

(vi) Coupling of the Cargo [Compound d] to the Delivery Carrier [[Modules (a)+(b)]+(c)]

To prepare the delivery conjugate as shown in FIG. 26, the delivery carrier, (25 nmol, based on CTB pentamer) from Example 30 (v) above in 250 μl of 250 μM EDTA, PBS is mixed with 100 nmol of the adapter-siRNA component (cargo) from Example 30(iv) above in 250 μL PBS, 250 μM EDTA and kept for 24 h at RT. The desired conjugate is purified by preparative SEC on a HiLoad™ 16/60 Superdex™ 200 prep grade column (GE Healthcare, part no. 17-1069-01) eluted at 1 mL/min with sterile PBS, pH 7.4. The column effluent is monitored at 260 nm and 550 nm. Calibration of the column is carried out prior to the preparative purification using the individual reaction components. Those fractions containing the desired conjugate are combined and concentrated by ultrafiltration using a Vivaspin 20 concentrator (30 kDa MWCO, Sartorius Stedim Biotech product no. VS2021) and the final concentrate is stored at 4° C. QC is performed by native gel electrophoresis and analytical SEC on a Superdex™ 200 10/300 GL column (GE Healthcare, part no. 17-5175-01). This procedure applies to both GAPDH and fLuciferase siRNAs.

### Example (31)

### Synthesis of DARE™ Delivery System Delivery Modules and Preparation of the Modules-siRNA Conjugate DARE™-CTB-(-MYCIGMu)(-siRNA)

(i) Synthesis and Activation of Module (c)

A module (c) molecule: H₂N—C(NPyS)SGSGSG-(DprAoa)SGSGSGEQKLISEEDL GKPTLYQVSLIMSDTGGTSY (module (c) comprises SEQ ID NO: 54 (IgMμ), SEQ ID NO: 305 (c-myc epitope tag), and two linker peptides each comprising SEQ ID NO:98 and providing dual link positions) is synthesized commercially by solid-phase Fmoc peptide chemistry. Deprotection is performed in the standard fashion and the crude product is purified by reversed phase HPLC to give a purity >95%. The activated cysteine residue is introduced using Boc-Cys(NPys)-OH (Bachem product no. A-2825) as a building block. Fmoc-Dpr(Boc-Aoa)-OH ((N-α-Fmoc-N-β-(N-t.-Boc-aminooxyacetyl)-L-diaminopropionic acid; Novabiochem product no. 04-12-1185) is used to introduce the N-β-aminooxyacetyl L-diaminopropionyl residue. Quality control (QC) of the purified peptide is done by amino acid analysis, electrospray mass spectroscopy (ESMS) and analytical reversed phase HPLC. To activate module (c), 600 nmol of (i) is transferred to an Eppendorf vial and treated for 1 h with 15 mg/mL DTT after which it is purified by preparative gel filtration [Size Exclusion Chromatography (SEC)] using a HiLoad™ 16/60 Superdex™ 75 prep grade column (GE Healthcare, part no. 17-1068-01) eluted with PBS (degassed with N₂) at a flow rate of 1 mL/min. Identification of the desired carrier peak is enabled by having calibrated the SEC column with module (c). Product containing fractions are pooled and lyophilised. Deprotected module (c) is employed immediately in step (iii).

Alternatively module (c) is treated for 1 h with 15 mg/mL DTT and transferred to a PD Miditrap™ G-10 (GE Healthcare 28-9180-11) pre-equilibrated with PBS, 250 μM EDTA. The fraction containing the deprotected module (c) is employed immediately in step (iii).

(ii) Functionalization of Module (a):

To functionalize module [(a)+(b)], 5.8 mg (100 nmol of pentamer) of recombinant cholera toxin subunit B [(CTB; SEQ ID NO: 117), obtained from SBL Vaccin AB, Matfors, Sweden, see www.rctb.net/theproduct.htm] in 2.1 mL of sterile PBS is mixed with a fresh solution of 0.26 mg (0.5 μmol) of sulfosuccinimidyl 6-[3′-(2-pyridyldithio)-propionamido]hexanoate (sulfo-LC-SPDP) in 75 μL of sterile PBS pH 7.4 in a sterile 1 mL Eppendorf tube and kept for 0.5 h at room temperature with occasional mixing. The solution is then desalted and buffer exchanged against degassed 10 mM sodium phosphate buffer, 150 mM NaCl, pH 7.4 (PBS) containing 250 μM EDTA using a Vivaspin 6 centrifugal concentrator (10 kDa MWCO, Sartorius Stedim Biotech product no. VS0601). Initially the functionalized CTB solution is diluted with 5 mL of PBS plus 250 μM EDTA concentrated to a volume of 0.5 mL and then washed with 6×5 mL of PBS plus 250 μM EDTA at room temperature, each time reducing the volume to 0.5 mL. In order to determine the 2-pyridyl-disulfide loading of the functionalized CTB a small aliquot (10 μL) of the solution is diluted to 1 mL with PBS and the absorbance at 343 nm measured using a quartz cuvette with a 1 cm pathlength. A 10 μL aliquot of a 15 mg/mL solution of dithiothreitol (DTT) in PBS is added to the cuvette, the contents are mixed carefully and the absorbance at 343 nm is measured after 15 min at room temperature, enabling the amount of pyridine-2-thione released to be quantitated (ε₃₄₃ₙₘ is 8080 M⁻¹ cm⁻¹, i.e. 1 μmol is equivalent to 8.08 A₃₄₃ units). The 2-pyridyl-disulfide loading of the CTB is generally around 5 μmol per μmol of pentamer.

(iii) Synthesis of the Delivery Carrier Comprising Functionalized Module [(a)+(b)]+(c)

250 μL of solution containing functionalized CTB [(a)+(b)] (50 nmol) from Example 31 (ii) above is reacted for 18 h at RT under nitrogen with 1.44 mg (250 nmol) activated module (c) from Example 31(i) dissolved in 750 μL of 100 mM phosphate buffer pH 7.4. Following a brief centrifugation the desired carrier [modules [[(a)+(b)]+(c)] is then purified by preparative gel filtration [Size Exclusion Chromatography (SEC)] using a HiLoad™ 16/60 Superdex™ 75 prep grade column (GE Healthcare, part no. 17-1068-01) eluted with PBS at a flow rate of 1 mL/min. Identification of the desired carrier peak is enabled by having calibrated the SEC column with CTB and with module (c) from Example 31(i). Product containing fractions are pooled and concentrated to a volume of 0.5 mL using a Vivaspin 20 protein concentrator (10 kDa MWCO, Sartorius Stedim Biotech product no. VS2001).

(iv) Preparation and Activation of the Cargo siRNAs [Compounds d]

A double stranded RNA molecule comprised of two 21 mer strands, with a double stranded region of 19 nucleotides in length and 2 nucleotides overhanging at the 3′ end of each strand, and targeting glyceraldehyde 3-phosphate dehydrogenase (GAPDH), wherein the sense strand comprises CCAuCUUCCAGGAGCgAGAuu (SEQ ID NO: 194), wherein lowercase u or g represents a 2′-O-Me-modified nucleotide; and the antisense strand comprises UCUCGCUCCUGgAAGAuGGdTdG (SEQ ID NO: 195), wherein lowercase u or g represents a 2′-O-Me-modified nucleotide and wherein the antisense strand has a 5′-phosphate and deoxynucleotides at its 3′ end (dNdN), is synthesized such that the 5P-terminus of the sense strand is modified with 5′-(C6 aminolinker)-phosphate-(C6-SS—C6 spacer)-phosphate-Cy3. In addition, a double stranded RNA molecule comprised of two 21 mer strands, with a double stranded region of 19 nucleotides in length and 2 nucleotides overhanging at the 3′-end of each strand, and targeting firefly luciferase (fLuc), wherein the sense strand comprises 5′-CUUACgCUGAGuACUUCGAuu (SEQ ID NO: 197), wherein lowercase u or g represents a 2′-O-methylribonucleotide; and the antisense strand comprises 5′-UCGAAGUACUC AgCGUAAgdTdG (SEQ ID NO: 198), wherein lowercase g represents a 2′-O-methylribonucleotide and wherein the antisense strand has a 5′-phosphate and deoxynucleotides at its 3′-end (dNdN), is synthesized such that the 5′-terminus of the sense strand is modified with a 5-(C6-SS—C6 spacer)-phosphate-Cy3 moiety. The four HPLC-purified individual single strands are all analyzed by HPLC and MALDI-TOF MS. The Cy3 dye is for tracking purposes by fluorescence and the disulfide bond ensures that the cargo can finally be released within the reducing environment of the cell cytoplasm. The single strands are analyzed by ESMS and analytical HPLC for QC prior to annealing. The desalted lyophilized siRNA (200 nmol) is transferred to an Eppendorf vial and treated for 1 h with 15 mg/mL DTT. The deprotected siRNA is then desalted and buffer exchanged against degassed PBS, 250 μM EDTA using a Vivaspin 15 centrifugal concentrator (5 kDa MWCO, Sartorius Stedim Biotech product no. VS1511). Initially the activated siRNA solution is diluted with 5.0 mL of sterile degassed, 250 μM EDTA, concentrated to a volume of 1.5 mL and then washed with 5×5.0 mL (or until no apparent DTT odor can be smelled anymore) of sterile degassed 250 uM EDTA in PBS, each time reducing the volume to 1.5 mL. The deprotected siRNA is employed immediately in (vi), Example 31. This procedure applies to both GAPDH and fLuciferase siRNAs.

(v) Functionalization of the Delivery Carrier Comprising Module [[(a)+(b)]+(c)]

To functionalize module [[(a)+(b)]+(c)], 50 nmol of module [[(a)+(b)]+(c)], Example 31 (iii), in 0.50 mL of sterile PBS is mixed with 37.5 μL of a fresh stock solution of 0.26 mg (0.5 μmol) of sulfosuccinimidyl 6-[3′-(2-pyridyldithio)-propionamido]hexanoate (sulfo-LC-SPDP) in 75 μL of sterile PBS pH 7.4 in a sterile 1 mL Eppendorf tube and kept 1.5 h at room temperature with occasional mixing. The solution is then desalted and buffer exchanged against degassed 10 mM sodium phosphate buffer, 150 mM NaCl, pH 7.4 (PBS) containing 250 μM EDTA using a Vivaspin 6 centrifugal concentrator (10 kDa MWCO, Sartorius Stedim Biotech product no. VS0601). Initially the functionalized module [[(a)+(b)]+(c)] solution is diluted with 5 mL of PBS plus 250 μM EDTA concentrated to a volume of 0.5 mL and then washed with 6×5 mL of PBS plus 250 μM EDTA at room temperature, each time reducing the volume to 0.5 mL. In order to determine the 2-pyridyl-disulfide loading of the functionalized CTB a small aliquot (10 μL) of the solution is diluted to 1 mL with PBS and the absorbance at 343 nm measured using a quartz cuvette with a 1 cm pathlength. A 10 μL aliquot of a 15 mg/mL solution of dithiothreitol (DTT) in PBS is added to the cuvette, the contents are mixed carefully and the absorbance at 343 nm is measured after 15 min at room temperature, enabling the amount of pyridine-2-thione released to be quantitated (ε₃₄₃ₙₘ is 8080 M⁻¹ cm⁻¹, i.e. 1 μmol is equivalent to 8.08 A₃₄₃ units).

(vi) Coupling of the Cargo [Compound d] to the Delivery Carrier [Modules [(a)+(b)]+(c)]

To prepare the delivery conjugate as shown in FIG. 27, the delivery carrier, (25 nmol, based on CTB pentamer) from Example 31 (v) above in 250 μL of 250 μM EDTA, PBS is mixed with 100 nmol of the adapter-siRNA component (cargo) from Example 31(iv) above in 250 μl PBS, 250 μM EDTA and kept for 24 h at RT. The desired conjugate is purified by preparative SEC on a HiLoad™ 16/60 Superdex™ 200 prep grade column (GE Healthcare, part no. 17-1069-01) eluted at 1 mL/min with sterile PBS, pH 7.4. The column effluent is monitored at 260 nm and 550 nm. Calibration of the column is carried out prior to the preparative purification using the individual reaction components. Those fractions containing the desired conjugate are combined and concentrated by ultrafiltration using a Vivaspin 20 concentrator (30 kDa MWCO, Sartorius Stedim Biotech product no. VS2021) and the final concentrate is stored at 4° C. QC is performed by native gel electrophoresis and analytical SEC on a Superdex™ 200 10/300 GL column (GE Healthcare, part no. 17-5175-01). This procedure applies to both GAPDH and fLuciferase siRNAs.

### Example (32)

### Synthesis of DARE™ Delivery System Delivery Modules and Preparation of the Modules-siRNA Conjugate DARE™-CTB-COX2STEL-siRNA

Example 32 is almost identical to Example 28, except for the decoration of the outer surface of CTB with reduced SPDP instead of intact residual SPDP groups.

### Method A

(i) Synthesis of Module (c)

A module (c) molecule: H₂N—C(NPyS)SGSGSG-(DprAoa)SGSGSGNASSSR SGLDDINPTVLLKERSTEL [module (c) comprises SEQ ID NO: 44 (COX2STEL peptide) with dual link positions using two linker peptides each comprising SEQ ID NO: 98] is synthesized commercially by solid-phase Fmoc peptide chemistry. Deprotection is performed in the standard fashion and the crude product is purified by reversed phase HPLC to give a purity >95%. The activated cysteine residue is introduced using Boc-Cys(NPys)-OH (Bachem product no. A-2825) as a building block. Fmoc-Dpr(Boc-Aoa)-OH((N-α-Fmoc-N-β-(N-t.-Boc-aminooxyacetyl)-L-diaminopropionic acid; Novabiochem product no. 04-12-1185) is used to introduce the N-β-aminooxyacetyl L-diaminopropionyl residue. Quality control (QC) of the purified peptide is done by amino acid analysis, electrospray mass spectroscopy (ESMS) and analytical reversed phase HPLC.

(ii) Functionalization and Activation of Module [(a)+(b)]:

To functionalize module (a), 23.2 mg (400 nmol of pentamer) of recombinant cholera toxin subunit B [(CTB; SEQ ID NO: 117), obtained from SBL Vaccin AB, Matfors, Sweden, see www.rctb.net/theproduct.htm] in 0.5 mL of sterile PBS is mixed with a fresh solution of 2.1 mg (4 μmol) of sulfosuccinimidyl 6-[3′-(2-pyridyldithio)-propionamido] hexanoate (sulfo-LC-SPDP) in 300 μL of sterile PBS pH 7.4 in a sterile 1 mL Eppendorf tube and kept 1.5 h at room temperature with occasional mixing. The solution is then desalted and buffer exchanged against degassed 10 mM sodium phosphate buffer, 150 mM NaCl, pH 7.4 (PBS) containing 250 μM EDTA using a Vivaspin 6 centrifugal concentrator (10 kDa MWCO, Sartorius Stedim Biotech product no. VS0601). Initially the functionalized CTB solution is diluted with 5 mL of PBS plus 250 μM EDTA concentrated to a volume of 0.5 mL and then washed with 6×5 mL of PBS plus 250 μM EDTA at room temperature, each time reducing the volume to 0.5 mL. In order to determine the 2-pyridyl-disulfide loading of the functionalized CTB a small aliquot (10 up of the solution is diluted to 1 mL with PBS and the absorbance at 343 nm measured using a quartz cuvette with a 1 cm pathlength. A 10 μL aliquot of a 15 mg/mL solution of dithiothreitol (DTT) in PBS is added to the cuvette, the contents are mixed carefully and the absorbance at 343 nm is measured after 15 min at room temperature, enabling the amount of pyridine-2-thione released to be quantitated (ε₃₄₃ₙₘ is 8080 M⁻¹ cm⁻¹, i.e. 1 mmol is equivalent to 8.08 A₃₄₃ units). The 2-pyridyl-disulfide loading of the CTB is generally around 5 μmol per μmol of pentamer. It is clear to one skilled in the art that the loading can be reduced by reducing the excess of sulfo-LC-SPDP used in the functionalization.

The SPDP functionalized CTB, 125 μL (100 nmol) is transferred to an Eppendorf vial and treated for 1 h with 15 mg/mL DTT. The thiol activated CTB is then desalted and buffer exchanged against degassed PBS, 250 μM EDTA using a Vivaspin 15 centrifugal concentrator (5 kDa MWCO, Sartorius Stedim Biotech product no. VS1511). Initially the activated CTB solution is diluted with 5.0 mL of sterile degassed, 250 μM EDTA, concentrated to a volume of 1.5 mL and then washed with 5×5.0 mL (or until no apparent DTT odor can be smelled anymore) of sterile degassed 250 μM EDTA in PBS, each time reducing the volume to 0.5 mL. The activated CTB is employed immediately in (iii) or (vii), Example 32.

(iii) Synthesis of the Delivery Carrier Comprising Functionalized Module [(a)+(b)]+(c)

250 μL of solution containing activated CTB (50 nmol) from Example 32(ii) above is reacted for 18 h at RT under nitrogen with 1.0 mg (250 nmol) of the linkage molecule containing module (c) from Example 32(i) above dissolved in 750 μL of 100 mM phosphate buffer pH 7.4. Following a brief centrifugation the desired carrier [modules (a)+(c)] is then purified by preparative gel filtration [Size Exclusion Chromatography (SEC)] using a HiLoad™ 16/60 Superdex™ 75 prep grade column (GE Healthcare, part no. 17-1068-01) eluted with PBS at a flow rate of 1 mL/min. Identification of the desired carrier peak is enabled by having calibrated the SEC column with CTB and with the linker-peptide entity from Example 32(i). Product containing fractions are pooled and concentrated to a volume of 0.5 mL using a Vivaspin 20 protein concentrator (10 kDa MWCO, Sartorius Stedim Biotech product no. VS2001).

(iv)Preparation of the Cargo siRNAs [Module d]

A double stranded RNA molecule comprised of two 21 mer strands, with a double stranded region of 19 nucleotides in length and 2 nucleotides overhanging at the 3′ end of each strand, and targeting glyceraldehyde 3-phosphate dehydrogenase (GAPDH), wherein the sense strand comprises CCAuCUUCCAGGAGCgAGAuu (SEQ ID NO: 194), wherein lowercase u or g represents a 2′-O-Me-modified nucleotide; and the antisense strand comprises UCUCGCUCCUGgAAGAuGGdTdG (SEQ ID NO: 195), wherein lowercase u or g represents a 2′-O-Me-modified nucleotide and wherein the antisense strand has a 5′-phosphate and deoxynucleotides at its 3′ end (dNdN), is synthesized such that the 5′-terminus of the sense strand is modified with 5′-(C6 aminolinker)-phosphate-(C6-SS—C6 spacer)-phosphate-Cy3. In addition, a double stranded RNA molecule comprised of two 21 mer strands, with a double stranded region of 19 nucleotides in length and 2 nucleotides overhanging at the 3′-end of each strand, and targeting firefly luciferase (fLuc), wherein the sense strand comprises 5′-CUUACgCUGAGuACUUCGAuu (SEQ ID NO: 197), wherein lowercase u or g represents a 2′-O-methylribonucleotide; and the antisense strand comprises 5′-UCGAAGUACUC AgCGUAAgdTdG (SEQ ID NO: 198), wherein lowercase g represents a 2′-β-methylribonucleotide and wherein the antisense strand has a 5′-phosphate and deoxynucleotides at its 3′-end (dNdN), is synthesized such that the 5′-terminus of the sense strand is modified with a 5-(C6-SS—C6 spacer)-phosphate-Cy3 moiety. The four HPLC-purified individual single strands are all analyzed by HPLC and MALDI-TOF MS. The Cy3 dye is for tracking purposes by fluorescence and the disulfide bond ensures that the cargo can finally be released within the reducing environment of the cell cytoplasm. The single strands are analyzed by ESMS and analytical HPLC for QC prior to annealing. The desalted lyophilized siRNA (200 nmol) is dissolved in 1 mL of sterile 100 mM sodium tetraborate buffer pH 8.5 and reacted with 200 μL of a fresh solution of 3.49 mg (10 μmol) of sulfosuccinimidyl 4-formylbenzoate (sulfo-S-4FB, SoluLink, product no. S-1008-105) dissolved in 500 μL of sterile 100 mM sodium tetraborate buffer, pH 8.5 for 3 h at RT. The siRNA bearing a benzaldehyde function is then desalted and buffer exchanged against degassed 100 mM sodium phosphate buffer, 150 mM NaCl, 500 μM EDTA, pH 6 using a Vivaspin 15 centrifugal concentrator (5 kDa MWCO, Sartorius Stedim Biotech product no. VS1511). Initially the benzaldehyde modified siRNA solution is diluted with 8.5 mL of sterile degassed 100 mM sodium phosphate, 150 mM sodium chloride, 500 μM EDTA, pH 6 concentrated to a volume of 1.5 mL and then washed with 5×8.5 mL of sterile degassed 100 mM sodium phosphate, 150 mM sodium chloride, 500 μM EDTA, pH 6 at room temperature, each time reducing the volume to 1.5 mL. QC of the benzaldehyde modified siRNA is done by ESMS and analytical HPLC. A small aliquot of the sample is analyzed for the presence of the aldehyde moiety by reaction with an excess of Cascade Blue hydrazide (Molecular Probes, catalog no. C-687) in buffer at pH 5, desalted by ethanol precipitation and analyzed by native anion-exchange HPLC on a MonoQ column (GE Healthcare) using multiwavelength detection (260 nm for the RNA, 399 nm for the Cascade Blue and 550 nm for the Cy3). This procedure applies to both GAPDH and fLuciferase.

(v) Coupling of the Cargo [Compound d] to the Delivery Carrier [[Modules (a)+(b)]+(c)]

The delivery carrier (40 nmol, based on CTB pentamer) from Example 32 (iii) above in 500 μL of 100 mM phosphate buffer containing 150 mM NaCl, 100 mM aniline pH 7 is mixed with 200 nmol of the adapter-siRNA component (cargo) from Example 32(iv) above in 500 μL of 100 mM phosphate buffer containing 150 mM NaCl, 100 mM aniline and kept for 48 h at RT. The desired conjugate is purified by preparative SEC on a HiLoad™ 16/60 Superdex™ 200 prep grade column (GE Healthcare, part no. 17-1069-01) eluted at 1 mL/min with sterile PBS, pH 7.4. The column effluent is monitored at 260 nm and 550 nm. Calibration of the column is carried out prior to the preparative purification using the individual reaction components. Those fractions containing the desired conjugate are combined and concentrated by ultrafiltration using a Vivaspin 20 concentrator (30 kDa MWCO, Sartorius Stedim Biotech product no. VS2021) and the final concentrate is stored at 4° C. QC is performed by native gel electrophoresis and analytical SEC on a Superdex™ 200 10/300 GL column (GE Healthcare, part no. 17-5175-01). This procedure applies to both GAPDH and fLuciferase.

### Method B

(vi) Coupling of the Cargo siRNAs [Compound d] to Module (c)

A benzaldehyde modified siRNA (300 nmol), Example 32 (iv) in 500 μL of 100 mM phosphate buffer containing 150 mM NaCl, 100 mM aniline pH 7 is mixed with 300 nmol of module (c) from Example 32(i) and kept for 48 h at RT. The desired conjugate is purified by preparative SEC on a HiLoad™ 16/60 Superdex™ 75 prep grade column (GE Healthcare, part no. 17-1068-01) eluted at 1 mL/min with sterile PBS, pH 7.4. The column effluent is monitored at 260 nm and 550 nm. Calibration of the column is carried out prior to the preparative purification using the individual reaction components. Those fractions containing the desired conjugate are combined and concentrated by ultrafiltration using a Vivaspin 20 concentrator (30 kDa MWCO, Sartorius Stedim Biotech product no. VS2021) and the final concentrate is stored at 4° C. QC is performed by native gel electrophoresis and analytical SEC on a Superdex™ 200 10/300 GL column (GE Healthcare, part no. 17-5175-01). This procedure applies to both GAPDH and fLuciferase siRNAs.

(vii) Coupling of Activated Module [(a)+(b)] to [(c)-Cargo siRNAs]

To prepare the delivery conjugate of FIG. 28, the activated CTB (50 nmol, based on CTB pentamer) from Example 32 (ii) above in 500 μL degassed (N₂) PBS, 250 μM EDTA is mixed with 250 nmol of the module (c)-siRNA from Example 32(vi) above in 500 μL of PBS and kept for 18 h at RT. The desired conjugate is purified by preparative SEC on a HiLoad™ 16/60 Superdex™ 200 prep grade column (GE Healthcare, part no. 17-1069-01) eluted at 1 mL/min with sterile PBS, pH 7.4. The column effluent is monitored at 260 nm and 550 nm. Calibration of the column is carried out prior to the preparative purification using the individual reaction components. Those fractions containing the desired conjugate are combined and concentrated by ultrafiltration using a Vivaspin 20 concentrator (30 kDa MWCO, Sartorius Stedim Biotech product no. VS2021) and the final concentrate is stored at 4° C. QC is performed by native gel electrophoresis and analytical SEC on a Superdex™ 200 10/300 GL column (GE Healthcare, part no. 17-5175-01). This procedure applies to both GAPDH and fLuciferase.

### Example (33)

### Synthesis of DARE™ Delivery System Delivery Modules and Preparation of the Modules-siRNA Conjugate DARE™-CTB-MYCIGMu-siRNA

Example 33 is almost identical to Example 29, except for the decoration on the outer surface of CTB with reduced SPDP instead of intact residual SPDP groups.

### Method A

(i) Synthesis of Module (c)

A module (c) molecule: H₂N—C(NPyS)SGSGSG-(DprAoa)SGSGSGEQKLISEEDL GKPTLYQVSLIMSDTGGTSY (module (c) comprises SEQ ID NO: 54 (IgMn), SEQ ID NO: 305 (c-myc epitope tag), and two linker peptides each comprising SEQ ID NO:98 and providing dual link positions) is synthesized commercially by solid-phase Fmoc peptide chemistry. Deprotection is performed in the standard fashion and the crude product is purified by reversed phase HPLC to give a purity >95%. The activated cysteine residue is introduced using Boc-Cys(NPys)-OH (Bachem product no. A-2825) as a building block. Fmoc-Dpr(Boc-Aoa)-OH ((N-α-Fmoc-N-β-(N-t.-Boc-aminooxyacetyl)-L-diaminopropionic acid; Novabiochem product no. 04-12-1185) is used to introduce the N-β-aminooxyacetyl L-diaminopropionyl residue. Quality control (QC) of the purified peptide is done by amino acid analysis, electrospray mass spectroscopy (ESMS) and analytical reversed phase HPLC.

(ii) Functionalization and Activation of Module [(a)+(b)]:

To functionalize module [(a)+(b)], 23.2 mg (400 nmol of pentamer) of recombinant cholera toxin subunit B [(CTB; SEQ ID NO: 117), obtained from SBL Vaccin AB, Matfors, Sweden, see www.rctb.net/theproduct.htm] in 0.5 mL of sterile PBS is mixed with a fresh solution of 2.1 mg (4 mmol) of sulfosuccinimidyl 6-[3′-(2-pyridyldithio)-propionamido]hexanoate (sulfo-LC-SPDP) in 300 μL of sterile PBS pH 7.4 in a sterile 1 mL Eppendorf tube and kept 1.5 h at room temperature with occasional mixing. The solution is then desalted and buffer exchanged against degassed 10 mM sodium phosphate buffer, 150 mM NaCl, pH 7.4 (PBS) containing 250 μM EDTA using a Vivaspin 6 centrifugal concentrator (10 kDa MWCO, Sartorius Stedim Biotech product no. VS0601). Initially the functionalized CTB solution is diluted with 5 mL of PBS plus 250 μM EDTA concentrated to a volume of 0.5 mL and then washed with 6×5 mL of PBS plus 250 μM EDTA at room temperature, each time reducing the volume to 0.5 mL. In order to determine the 2-pyridyl-disulfide loading of the functionalized CTB a small aliquot (10 μL) of the solution is diluted to 1 mL with PBS and the absorbance at 343 nm measured using a quartz cuvette with a 1 cm pathlength. A 10 μA aliquot of a 15 mg/mL solution of dithiothreitol (DTT) in PBS is added to the cuvette, the contents are mixed carefully and the absorbance at 343 nm is measured after 15 min at room temperature, enabling the amount of pyridine-2-thione released to be quantitated (ε₃₄₃ₙₘ is 8080 M⁻¹ cm⁻¹, i.e. 1 μmol is equivalent to 8.08 A₃₄₃ units). The 2-pyridyl-disulfide loading of the CTB is generally around 5 μmol per μmol of pentamer. It is clear to one skilled in the art that the loading can be reduced by reducing the excess of sulfo-LC-SPDP used in the functionalization.

The SPDP functionalized CTB, 125 μL (100 nmol) is transferred to an Eppendorf vial and treated for 1 h with 15 mg/mL DTT. The thiol activated CTB is then desalted and buffer exchanged against degassed PBS, 250 μM EDTA using a Vivaspin 15 centrifugal concentrator (5 kDa MWCO, Sartorius Stedim Biotech product no. VS1511). Initially the activated CTB solution is diluted with 5.0 mL of sterile degassed, 250 μM EDTA, concentrated to a volume of 1.5 mL and then washed with 5×5.0 mL (or until no apparent DTT odor can be smelled anymore) of sterile degassed 250 μM EDTA in PBS, each time reducing the volume to 0.5 mL. The activated CTB is employed immediately in (iii) or (vii), Example 33.

(iii) Synthesis of the Delivery Carrier Comprising Functionalized Module [[(a)+(b)+(c)]

250 μL of solution containing activated CTB [(a)+(b)] (50 nmol) from Example 33(ii) above is reacted for 18 h at RT under nitrogen with 1.0 mg (250 nmol) of the linkage molecule containing module (c) from Example 33(i) above dissolved in 750 μL of 100 mM phosphate buffer pH 7.4. Following a brief centrifugation the desired carrier [modules [[(a)+(b)]+(c)] is then purified by preparative gel filtration [Size Exclusion Chromatography (SEC)] using a HiLoad™ 16/60 Superdex™ 75 prep grade column (GE Healthcare, part no. 17-1068-01) eluted with PBS at a flow rate of 1 mL/min. Identification of the desired carrier peak is enabled by having calibrated the SEC column with CTB and with the linker-peptide entity from Example 33(i). Product containing fractions are pooled and concentrated to a volume of 0.5 mL using a Vivaspin 20 protein concentrator (10 kDa MWCO, Sartorius Stedim Biotech product no. VS2001).

(iv)Preparation of the Cargo siRNAs [module d]

A double stranded RNA molecule comprised of two 21 mer strands, with a double stranded region of 19 nucleotides in length and 2 nucleotides overhanging at the 3′ end of each strand, and targeting glyceraldehyde 3-phosphate dehydrogenase (GAPDH), wherein the sense strand comprises CCAuCUUCCAGGAGCgAGAuu (SEQ ID NO: 194), wherein lowercase u or g represents a 2′-O-Me-modified nucleotide; and the antisense strand comprises UCUCGCUCCUGgAAGAuGGdTdG (SEQ ID NO: 195), wherein lowercase u or g represents a 2′-O-Me-modified nucleotide and wherein the antisense strand has a 5′-phosphate and deoxynucleotides at its 3′ end (dNdN), is synthesized such that the 5P-terminus of the sense strand is modified with 5′-(C6 aminolinker)-phosphate-(C6-SS—C6 spacer)-phosphate-Cy3. In addition, a double stranded RNA molecule comprised of two 21 mer strands, with a double stranded region of 19 nucleotides in length and 2 nucleotides overhanging at the 3′-end of each strand, and targeting firefly luciferase (fLuc), wherein the sense strand comprises 5′-CUUACgCUGAGuACUUCGAuu (SEQ ID NO: 197), wherein lowercase u or g represents a 2′-O-methylribonucleotide; and the antisense strand comprises 5′-UCGAAGUACUC AgCGUAAgdTdG (SEQ ID NO: 198), wherein lowercase g represents a 2′-O-methylribonucleotide and wherein the antisense strand has a 5′-phosphate and deoxynucleotides at its 3′-end (dNdN), is synthesized such that the 5′-terminus of the sense strand is modified with a 5-(C6-SS—C6 spacer)-phosphate-Cy3 moiety. The four HPLC-purified individual single strands are all analyzed by HPLC and MALDI-TOF MS. The Cy3 dye is for tracking purposes by fluorescence and the disulfide bond ensures that the cargo can finally be released within the reducing environment of the cell cytoplasm. The single strands are analyzed by ESMS and analytical HPLC for QC prior to annealing. The desalted lyophilized siRNA (200 nmol) is dissolved in 1 mL of sterile 100 mM sodium tetraborate buffer pH 8.5 and reacted with 200 μL of a fresh solution of 3.49 mg (10 mmol) of sulfosuccinimidyl 4-formylbenzoate (sulfo-S-4FB, SoluLink, product no. S-1008-105) dissolved in 500 μL of sterile 100 mM sodium tetraborate buffer, pH 8.5 for 3 h at RT. The siRNA bearing a benzaldehyde function is then desalted and buffer exchanged against degassed 100 mM sodium phosphate buffer, 150 mM NaCl, 500 μM EDTA, pH 6 using a Vivaspin 15 centrifugal concentrator (5 kDa MWCO, Sartorius Stedim Biotech product no. VS1511). Initially the benzaldehyde modified siRNA solution is diluted with 8.5 mL of sterile degassed 100 mM sodium phosphate, 150 mM sodium chloride, 500 μM EDTA, pH 6 concentrated to a volume of 1.5 mL and then washed with 5×8.5 mL of sterile degassed 100 mM sodium phosphate, 150 mM sodium chloride, 500 μM EDTA, pH 6 at room temperature, each time reducing the volume to 1.5 mL. QC of the benzaldehyde modified siRNA is done by ESMS and analytical HPLC. A small aliquot of the sample is analyzed for the presence of the aldehyde moiety by reaction with an excess of Cascade Blue hydrazide (Molecular Probes, catalog no. C-687) in buffer at pH 5, desalted by ethanol precipitation and analyzed by native anion-exchange HPLC on a MonoQ column (GE Healthcare) using multiwavelength detection (260 nm for the RNA, 399 nm for the Cascade Blue and 550 nm for the Cy3). This procedure applies to both GAPDH and fLuciferase.

(v) Coupling of the Cargo [Compound d] to the Delivery Carrier [Modules [[(a)+(b)]+(c)]]

The delivery carrier (40 nmol, based on CTB pentamer) from Example 33 (iii) above in 500 μL, of 100 mM phosphate buffer containing 150 mM NaCl, 100 mM aniline pH 7 is mixed with 200 nmol of the adapter-siRNA component (cargo) from Example 33(iv) above in 500 μL, of 100 mM phosphate buffer containing 150 mM NaCl, 100 mM aniline and kept for 48 h at RT. The desired conjugate is purified by preparative SEC on a HiLoad™ 16/60 Superdex™ 200 prep grade column (GE Healthcare, part no. 17-1069-01) eluted at 1 mL/min with sterile PBS, pH 7.4. The column effluent is monitored at 260 nm and 550 nm. Calibration of the column is carried out prior to the preparative purification using the individual reaction components. Those fractions containing the desired conjugate are combined and concentrated by ultrafiltration using a Vivaspin 20 concentrator (30 kDa MWCO, Sartorius Stedim Biotech product no. VS2021) and the final concentrate is stored at 4° C. QC is performed by native gel electrophoresis and analytical SEC on a Superdex™ 200 10/300 GL column (GE Healthcare, part no. 17-5175-01). This procedure applies to both GAPDH and fLuciferase.

### Method B

(vi) Coupling of the Cargo siRNAs [Compound d] to Module (c)

A benzaldehyde modified siRNA (300 nmol), Example 33 (iv) in 500 μL of 100 mM phosphate buffer containing 150 mM NaCl, 100 mM aniline pH 7 is mixed with 300 nmol of module (c) from Example 33(i) and kept for 48 h at RT. The desired conjugate is purified by preparative SEC on a HiLoad™ 16/60 Superdex™ 75 prep grade column (GE Healthcare, part no. 17-1068-01) eluted at 1 mL/min with sterile PBS, pH 7.4. The column effluent is monitored at 260 nm and 550 nm. Calibration of the column is carried out prior to the preparative purification using the individual reaction components. Those fractions containing the desired conjugate are combined and concentrated by ultrafiltration using a Vivaspin 20 concentrator (30 kDa MWCO, Sartorius Stedim Biotech product no. VS2021) and the final concentrate is stored at 4° C. QC is performed by native gel electrophoresis and analytical SEC on a Superdex™ 200 10/300 GL column (GE Healthcare, part no. 17-5175-01). This procedure applies to both GAPDH and fLuciferase siRNAs.

(vii) Coupling of Activated Module [(a)+(b)] to [(c)-Cargo siRNAs]

To prepare the delivery conjugate as shown in FIG. 29, the activated CTB (50 nmol, based on CTB pentamer) from Example 33 (ii) above in 500 μL degassed (N₂) PBS, 250 μM EDTA is mixed with 250 nmol of the module (c)-siRNA from Example 33(vi) above in 500 μL of PBS and kept for 18 h at RT. The desired conjugate is purified by preparative SEC on a HiLoad™ 16/60 Superdex™ 200 prep grade column (GE Healthcare, part no. 17-1069-01) eluted at 1 mL/min with sterile PBS, pH 7.4. The column effluent is monitored at 260 nm and 550 nm. Calibration of the column is carried out prior to the preparative purification using the individual reaction components. Those fractions containing the desired conjugate are combined and concentrated by ultrafiltration using a Vivaspin 20 concentrator (30 kDa MWCO, Sartorius Stedim Biotech product no. VS2021) and the final concentrate is stored at 4° C. QC is performed by native gel electrophoresis and analytical SEC on a Superdex™ 200 10/300 GL column (GE Healthcare, part no. 17-5175-01). This procedure applies to both GAPDH and fLuciferase.

### Example (34)

### Synthesis of DARE™ Delivery System Delivery Modules and Preparation of the Modules-siRNA Conjugate DARE™-CTB-CTA2-siRNA

(i) Synthesis of the Linker CTA2.

The linker CTA2 molecule: SEQ ID NO: 310; Ac-(L-propargylglycyl)-MSNTSDEKTQSLGVKFLDEYQSKVKRQIFSGYQSDIDTHNRIKDEL is synthesized commercially by solid-phase Fmoc peptide chemistry. The linker also comprises a natural KDEL motive and, thus, comprises a module (b). Deprotection is performed in the standard fashion and the crude product is purified by reversed phase HPLC to give a purity >95%. The L-propargylglycine is introduced using Fmoc-L-propargylglycine-OH (Novabiochem product no. 852360) as a building block. Quality control (QC) of the purified peptide is done by amino acid analysis, electrospray mass spectroscopy (ESMS) and analytical reversed phase HPLC.

(ii) Synthesis of the Delivery Carrier by Non-Covalent Assembly of Module [(a)+(b)+CTA2 Linker]

23.2 mg (400 nmol of pentamer) of recombinant cholera toxin subunit B [(CTB; SEQ ID NO: 117), obtained from SBL Vaccin AB, Matfors, Sweden, see www.rctb.net/theproduct.htm] in PBS was buffer exchanged with 100 mM glycine-HCl buffer, 6.5 M urea, pH 3.2 in a Vivaspin 6 centrifugal concentrator (3 kDa MWCO, Sartorius Stedim Biotech product no VS0691). Initially the CTB solution is diluted with 8.5 mL of 100 mM glycine-HCl buffer, 6.5 M urea, pH 3.2 and concentrated to a volume of 2.0 mL and then washed with 6×8.5 mL 100 mM glycine-HCl buffer, 6.5 M urea, pH 3.2 at room temperature, each time reducing the volume to 2.0 mL. Module (b) (4.4 mg, 800 nmol) in 1 mL of 100 mM glycine-HCl buffer, 6.5 M urea, pH 3.2 is added and the mixture is stirred for 1 h at RT. This solution is then dialyzed against sterile PBS, pH 7 so as to assemble the mutant CT holotoxin molecule containing one modified A2 peptide and five B subunits. The delivery carrier is purified by preparative SEC on a HiLoad™ 16/60 Superdex™ 75 prep grade column (GE Healthcare, part no. 17-1068-01) eluted at 1 mL/min with sterile PBS, pH 7.4. The column effluent is monitored at 220 and 280 nm. Those fractions containing the desired delivery carrier are combined and concentrated to 0.5 mL by ultrafiltration using a Vivaspin 15R concentrator (30 kDa MWCO, Sartorius Stedim Biotech product no. VS15RH21) and the final concentrate is stored at 4° C. QC is performed by native gel electrophoresis and analytical SEC on a Superdex 200 10/300 GL column (GE Healthcare, part no. 17-5175-01).

(iii) Preparation of the Cargo siRNAs [Compounds (d)]

A double stranded RNA molecule comprised of two 21 mer strands, with a double stranded region of 19 nucleotides in length and 2 nucleotides overhanging at the 3′ end of each strand, and targeting glyceraldehyde 3-phosphate dehydrogenase (GAPDH), wherein the sense strand comprises CCAuCUUCCAGGAGCgAGAuu (SEQ ID NO: 194), wherein lowercase u or g represents a 2′-O-Me-modified nucleotide; and the antisense strand comprises UCUCGCUCCUGgAAGAuGGdTdG (SEQ ID NO: 195), wherein lowercase u or g represents a 2′-O-Me-modified nucleotide and wherein the antisense strand has a 5′-phosphate and deoxynucleotides at its 3′ end (dNdN), is synthesized such that the 5P-terminus of the sense strand is modified with 5′-(C6 aminolinker)-phosphate-(C6-SS—C6 spacer)-phosphate-Cy3. In addition, a double stranded RNA molecule comprised of two 21 mer strands, with a double stranded region of 19 nucleotides in length and 2 nucleotides overhanging at the 3′-end of each strand, and targeting firefly luciferase (fLuc), wherein the sense strand comprises 5′-CUUACgCUGAGuACUUCGAuu (SEQ ID NO: 197), wherein lowercase u or g represents a 2′-O-methylribonucleotide; and the antisense strand comprises 5′-UCGAAGUACUCAgCGUAAgdTdG (SEQ ID NO: 198), wherein lowercase g represents a 2′-O-methylribonucleotide and wherein the antisense strand has a 5′-phosphate and deoxynucleotides at its 3′-end (dNdN), is synthesized such that the 5′-terminus of the sense strand is modified with 5′-(C6-SS—C6 spacer)-phosphate-Cy3. The four HPLC-purified individual single strands are analyzed by HPLC and MALDI-TOF MS. The Cy3 dye is for tracking purposes by fluorescence and the disulfide bond ensures that the cargo can finally be released within the reducing environment of the cell cytoplasm. The single strands are analyzed by ESMS and analytical HPLC for QC prior to annealing.

(iv) Preparation of Modified Cargo siRNAs [Modified Module d]

The desalted lyophilized siRNA (200 nmol) is dissolved in 1 mL of PBS and transferred to an Eppendorf tube containing 10 equivalents (0.45 mg, 2 mmol) of 4-azidobutyric acid NHS ester (Interchim product no. ZL5542) in 200 μL PBS, pH 7.4. After 1 h of moderate shaking at room temperature the azido modified siRNA is then desalted and buffer exchanged against degassed 100 mM sodium phosphate buffer, 150 mM NaCl, pH 6 using a Vivaspin 15 centrifugal concentrator (5 kDa MWCO, Sartorius Stedim Biotech product no. VS1511). Initially the modified siRNA solution is diluted with 8.5 mL of sterile degassed 100 mM sodium phosphate, 150 mM sodium chloride, pH 6 concentrated to a volume of 1.5 mL and then washed with 5×8.5 mL of sterile degassed 100 mM sodium phosphate, 150 mM sodium chloride, pH 6 at room temperature, each time reducing the volume to 1.5 mL. QC of the azido modified siRNA is done by ESMS and analytical HPLC. This procedure applies to both GAPDH and fLuciferase.

(v) Coupling of the Modified Cargo to Module [(a)+(b)+CTA2 Linker]

The modified cargo siRNA (200 nmol) from Example 34 (iv) above in 500 μL of nitrogen-degassed click buffer (1:1 PBS/DMSO, 0.5 mM copper(II)-TBTA complex and 0.5 mM ascorbic acid) is mixed with 150 nmol of module [(a)+(b)+CTA2 linker] from Example 34(ii) in 500 μL of the click buffer and kept for 18 h at RT. The resulting mixture is then desalted and buffer exchanged against degassed 100 mM sodium phosphate buffer, 150 mM NaCl, 1 mM EDTA, pH 6 using a Vivaspin 15 centrifugal concentrator (5 kDa MWCO, Sartorius Stedim Biotech product no. VS1511) by first diluting the solution with 8.5 mL of sterile degassed 100 mM sodium phosphate, 150 mM sodium chloride, 1 mM EDTA, pH 6 concentrated to a volume of 1.5 mL (repeat twice) and then washing with 5×8.5 mL of sterile degassed 100 mM sodium phosphate, 150 mM sodium chloride, pH 6 at room temperature, each time reducing the volume to 1.5 mL. The desired conjugate is purified by preparative SEC on a HiLoad™ 16/60 Superdex™ 200 prep grade column (GE Healthcare, part no. 17-1069-01) eluted at 1 mL/min with sterile PBS, pH 7.4. The column effluent is monitored at 260 nm and 550 nm. Calibration of the column is carried out prior to the preparative purification using the individual reaction components. Those fractions containing the desired conjugate are combined and concentrated by ultrafiltration using a Vivaspin 20 concentrator (30 kDa MWCO, Sartorius Stedim Biotech product no. VS2021) and the final concentrate is stored at 4° C. This procedure applies to both GAPDH and fLuciferase. The structure of the construct is shown schematically in FIG. 30.

### REFERENCES

1. Behlke, M A (2006). Mol Ther, 13:644-670.
2. Rozema, D. B., D. L. Lewis, et al. (2007). Proc Natl Acad Sci USA 104(32): 12982-7.
3. Sambrook et al. (1989). Molecular Cloning: A Laboratory Manual, 2d ed. Cold Spring Harbor Laboratory Press, Cold Spring Harbor, N.Y.
4. Leuenberger, H. G. W, Nagel, B. and Kölbl, H. eds. (1995). “A multilingual glossary of biotechnological terms: (IUPAC Recommendations).” Helvetica Chimica Acta, CH-4010 Basel, Switzerland.
5. Karlin and Altschul (1990). Proc. Natl. Acad. Sci. USA 87:2264-2268.
6. Karlin and Altschul (1993). Proc. Natl. Acad. Sci. USA 90:5873-5877.
7. Altschul, et al. (1990). J. Mol. Biol. 215:403-410.
8. Altschul et al. (1997). Nucleic Acids Res. 25:3389-3402.
9. Tung C-W and Ho S-Y, (2008). BMC Bioinformatics 9:310, 1-15.
10. Catic A., Collins C., Church G M, Ploegh HL (2004). Bioinformatics 20(18):3302-3307.
11. Xu P., et al. (2009). Cell 137 (1), 33-147.
12. Thrower, et al. (January 2000). EMBO 19 (1): 94-102.
13. Ivanov (Ed.) (2008). “Exocytosis and Endocytosis”, Series: Methods in Molecular Biology,
Springer Vol. 440, ISBN: 978-1-58829-865-2.
14. Ghosh et al. (1994). J. Cell Science 107:2177-2189.
15. Watson et al. (2005). Adv Drug Deliv Rev 57:43-61.
16. Mallard and Johannes. (2002). Methods Mol. Med. 2002. Shiga Toxin Methods and Protocols (Edited by: D Philpott and F Ebel), 73, (17), p 209-220.
17. Bernardi, et al. (2008). Mol. Biol. Cell 19:877-884.
18. Huston et al. (1988). Proc. Nat'l Acad. Sci. 85:5879.
19. Whitlow et al. (1993). Protein Engineering 6:989.
20. Newton et al. (1996). Biochemistry 35:545.
21. Eguchi, et al. (2009). Nat Biotechnol 27(6):567-71.
22. Bevilacqua and Cech. (1996). Biochemistry 35(31):9983-94.
23. McKenna et al. (2006). J Mol Biol 358(5):1270-85.
24. Tompa et al. (2004). J. Biol. Chem. 279(20):20775-20785.
25. Waehler et al. (2007). Nat Rev Genet. 8(8):573-587.
26. Tang et al. (2007). Gene Ther 14(6):523-532.
27. Tuma and Hubbard. (2003). Physiol Rev 83(3):871-932.
28. Zito et al. (2007). EMBO J. 26(10):2443-2453.
29. Lee et al. (2001). Eur J. Biochem. 268(7):2004-2012.
30. Xia et al. (2000). i Virol. 74(23):11359-11366.
31. Truskey et al. (2009). Transport Phenomena in Biological Systems, Chapter 11. “Cell Surface Ligand-Receptor Kinetics and Molecular Transport within Cells.” 2^{nd} Edition Pearson Education Inc., ISBN 978-O-13-156988-1.
32. Futter, et al. (1996). J. Cell Biol. 132:1011-1023.
33. Mallard et al. (1998) i Cell Biol. 143(4): 973-990
34. Johannes, et al. (1997). J. Biol. Chem. 272 (31):19554-19561.
35. Pelkmans, et al. (2001). Nat Cell Biol. 3(5):473-483.
36. Damm, et al. (2005). J. Cell Biol. 168(3):477-488.
37. Neu, et al. (2009). Virology. 384(2):389-399.
38. Raykhel et al. (2007). J. Cell Biol. 179(6):1193-1204.
39. Song and Smith. (1996). Arch Biochem Biophys. 334(1):67-72.
40. Johannes and Popoff (2008). Cell 135:1175-1187,
41. Sorgjerd et al. (2006). J Mol. Biol. 356(2):469-482.
42. Hirsch et al. (2009). Nature. 458(7237):453-460.
43. LaPointe et al. (2005). J Biol. Chem. 280(24):23310-23318.
44. Yue et al. (2010). Nature Chem. Biol. 6:621-629.
45. Agrawal. (1996). Trends in Biotech. 14:376-387.
46. Choi et al. (2006). Viral Immunol. 19(1):54-63.
47. Haicheur et al. (2000). J. Immunol. 165(6):3301-3308.
48. Smith et al. (2002). J. Immunol. 169(1):99-107.
49. Makkerh et al. (1996). Curr Biol. 6(8):1025-1027.
50. Mudhakir and Harashima (2009). AAPS J. 11(1):65-77.
51. Wade and Weller. (1994). “Handbook of Pharmaceutical Excipients”, 2nd Edition.
52. Berge et al. (1977). J. Pharm. Sci. 66:1-19.
53. Oslo (Ed.) (1985). Remington's Pharmaceutical Sciences. Mace Publishing Company, Philadelphia, Pa., 17th ed.
54. Langer. (1990). Science. 249:1527-1533.
55. Szoka et al. (1980). Ann. Rev. Biophys. Bioeng. 9:467.
56. Deamer, et al. (1983). In LIPOSOMES, Marcel Dekker, New York, p. 27
57. Hope, et al. (1986). Chem. Phys. Lipids. 40:89.
58. Santel et al. (2006). Gene Ther. 13(16):1222-1234.
59. Landen et al. (2005). Cancer Res. 65(15):6910-6918.
60. Spagnou et al. (2004). Biochemistry. 43(42):13348-133456.
61. Bouxsein et al. (2007). Biochemistry. 46(16):4785-4792.
62. Castanotto and Rossi, (2009). Nature. 457(7228):426-33.
63. Dominska and Dykxhoorn. (2010). J Cell Sci. 123(Pt 8):1183-1189.
64. Amarzguioui et al. (2006). Nat. Protoc. 1(2):508-517.
65. Liu et al. (2005). Neurobiol. 19(3):407-418.
66. Park et al. (2007). J Gene Med. 9(8):691-702.
67. d'Alessandro et al. (1998). Clin. Chim. Acta 274(2):189-197.
68. Sloots et al. (2005). FEBS J. 272:4221-4236.
69. Le et al. (2000). J Cell Sci. 113(18):3227-3240.
70. Le and Nabi. (2003). J Cell Sci. 116(6):1059-1071.
71. Bolte and Cordelieres. (2006). J Microsc. 224(3):213-232.
72. Manders et al. (1992). J Cell Sci. 103(3):857-862.
73. Li et al. (2004.) J. Neurosci. 24(16):4070-4081.
74. Zinchuk and Zinchuk. (2008). Curr Protoc Cell Biol. Chapter 4: p. Unit 4 19.
75. Vandesompele et al. (2002). Genome Biol. 3(7): RESEARCH 0034.
76. Svensson et al. (2008). Mol. Ther. 16(12):1995-2001.
77. Frank-Kamenetsky et al. (2008). Proc Natl Acad Sci USA. 105(33):11915-11920.
78. Sun et al. (2008). Nat. Biotechnol. 26(12): 1379-1382.
79. Judge et al. (2009). J Clin Invest. 119(3): 661-73.
80. Marti et al. (1998). Proc Natl Acad. Sci. U.S.A. 95(26):15809-15814.
81. Akinc et al. (2009). Mol. Ther. 17(5): 872-879.
82. Judge and MacLachlan. (2008). Hum Gene Ther. 19(2):111-124.
83. Christensen et al. (2006). Immunity. 25(3):417-428.
84. Forsbach et al. (2008). J. Immunol. 180(6):3729-3738.
85. Dalai et al. (2005). Clin Cancer Res. 11(6):2364-2378.
86. Gerber et al. (2000). Cancer Res. 60(22):6253-6258.
87. Borgstrom et al. (1996). Cancer Res. 56(17):4032-4039.
88. Mordenti et al. (1999). Toxicol Pathol. 27(1):14-21.
89. Takei et al. (2004). Cancer Res. 64(10):3365-3370.
90. Kim et al. (1993). Nature. 362(6423):841-844.
91. Presta et al. (1997). Cancer Res. 57(20):4593-4599.
92. Raskopf et al. (2008). J Hepatol. 49(6):977-984.
93. Guan et al. (2005). Clin. Cancer Res. 11(7):2662-2669.
94. Kleinman et al. (2008). Nature. 452(7187):591-597.
95. Kariko et al. (2005). Immunity. 23(2):165-175.
96. Judge et al. (2006). Mol. Ther. 13(3): 494-505.
97. Geisbert et al. (2006). J Infect Dis. 193(12):1650-1657.
98. Robbins et al. (2008). Hum Gene Ther. 19(10):991-999.
99. Marques et al. (2006). Nat. Biotechnol. 24(5):559-565.
100. Gerber et al. (1999). Development. 126(6):1149-1159.
101. Mu et al. (2009). Int J Cancer. 125(12):2978-2990.
102. Makin and Dive. (2001). Trends Cell Biol. 11(11):S22-26.
103. Takei et al. (2006). Cancer. 107(4):864-873.
104. Borowsky et al. (2005). Clin Exp Metastasis. 22(1):47-59.
105. Helms et al. (2010). Cancer Immunol Immunother. 59(9):1325-1334.
106. Watson and Paxinos (Eds.). (2007). “The rat brain in stereotaxic coordinates”. 6 ed. Elsevier Academic Press, London.
107. Paxinos and Franklin (Eds.). (2007). “The mouse brain in stereotaxic coordinates”. 3rd ed. Academic Press.
108. Thakker et al. (2004). Proc Natl Acad Sci USA. 101(49):17270-17275.
109. Thakker et al. (2005). Mol. Psychiatry. 10(8):782-789.
110. Guo et al. (2002). Oncogene. 21(49):7545-7556.
111. Wesche. (2002). Int J Med. Microbiol. 291(6-7):517-521.
112. Sandvig (1991). J. Cell Biol. 115(4):971-981.
113. Khine et al. (2004). Glycobiology. 14(8):701-712.
114. Arasaki et al. (2007). Mol. Pharmacol. 71(2):454-460.
115. Drecktrah et al. (1998). J Cell Sci. 111(Pt 7):951-965.
116. Stechmann et al. (2010). Cell. 141(2):231-242.
117. Saenz et al. (2009). Nat Chem. Biol. 5(3):157-165.

1. A conjugate for delivery of a compound into a cell comprising or consisting of:
(a) at least one module that mediates cell targeting and facilitates cellular uptake,
(b) at least one module that facilitates transport to the endoplasmic reticulum (ER),
(c) at least one module that mediates translocation from the ER to the cytosol, and
(d) at least one compound,
wherein module (a) is linked to module (c) or to module (b) through a linker; module (c) is linked to module (b) via a peptide linker and compound(s) (d) is(are) linked to the linker connecting module (a) and module (c) or module (b).

2. The conjugate of claim 1, wherein the modules and the compound are linked to each other in the following arrangement: (a)ₓ-(c)_{z}-(b)_{y} or (a)ₓ-(b)_{y}-(c)_{z} and compound(s) (d)ₙ; is(are) linked to the linker connecting module (a) and (c) or module (a) and (b) and wherein
x is an integer of 1 to 5, preferably of 1;
y is an integer of 1 to 5; preferably of 1;
z is an integer of 1 to 5; preferably of 1; and
n is an integer of 1 to 50, preferably of 1, 2, 3, 4, 5, 6, 7, 8, 9, or 10.

3. The conjugate of claim 2, wherein the arrangements in which the modules and the compound are linked to each other are (a)ₓ-(c)_{z}-(b)ₙ, or (a)ₓ-(b)ₙ-(c)_{z}, wherein x is an integer of 1, z is an integer of 1, and n is an integer of 1

4. The conjugate of claim 1, wherein the modules (a) and (c) or the modules (a) and (b) and/or the compound(s) (d) are
(i) linked to each other via a covalent linkage,
(ii) linked to each other via a non-covalent linkage,
(iii) linked to each other via at least one adapter molecule, and/or
(iv) linked to each other via at least one linker molecule that optionally comprises at least one adapter molecule.

5. The conjugate of claim 1, wherein the arrangements in which the modules and the compound are linked to each other are
(i) (a)ₓ, (c)_{z} and (b)_{y}, wherein (a)ₓ is covalently linked via a linker molecule to (c)_{z} and (c)_{z} is covalently linked directly or via a peptide linker to (b)_{y} and (d)ₙ is covalently linked to the linker molecule;
(ii) (a)ₓ, (c)_{z} and (b)_{y}, wherein (a)ₓ is covalently linked via a linker molecule to (c)_{z} and (c)_{z} is covalently linked directly or via a peptide linker to (b)_{y} and (d)ₙ is non-covalently linked to the linker molecule;
(iii) (a)ₓ, (c)_{z}, and (b)_{y}, wherein (a)ₓ is non-covalently linked via a linker molecule to (c)_{z} and (c)_{z} is covalently linked directly or via a peptide linker to (b)_{y} and (d)ₙ is covalently linked to the linker molecule; or
(iv) (a)ₓ, (c)_{z} and (b)_{y}, wherein (a)ₓ is non-covalently linked via a linker molecule to (c)_{z} and (c)_{z} is covalently linked directly or via a peptide linker to (b)_{y} and (d)ₙ is non-covalently linked to the linker molecule,
(v) (a)ₓ, (b)_{y} and (c)_{z}, wherein (a)ₓ is covalently linked via a linker molecule to (b)_{y} and (b)_{y} is covalently linked directly or via a peptide linker to (c)_{z} and (d)ₙ is covalently linked to the linker molecule;
(vi) (a)ₓ, (b)_{y} and (c)_{z}, wherein (a)ₓ is covalently linked via a linker molecule to (b)_{y} and (b)_{y} is covalently linked directly or via a peptide linker to (c)_{z} and (d)ₙ is non-covalently linked to the linker molecule;
(vii) (a)ₓ, (b)_{y} and (c)_{z}, wherein (a)ₓ is non-covalently linked via a linker molecule to (b)_{y} and (b)_{y} is covalently linked directly or via a peptide linker to (c)_{z} and (d)ₙ is covalently linked to the linker molecule; or
(viii) (a)ₓ, (b)_{y} and (c)_{z}, wherein (a)ₓ is non-covalently linked via a linker molecule to (b)_{y} and (b)_{y} is covalently linked directly or via a peptide linker to (c)_{z} and (d)ₙ is non-covalently linked to the linker molecule.

6. The conjugate of claim 4, wherein the covalent linkage is a disulfide-linkage, an amide-linkage, an oxime-linkage or a hydrazone-linkage and, wherein the non-covalent linkage is an ionic linkage or a hydrophobic linkage.

7. The conjugate of claim 4, wherein the linker molecule is a peptide, a modified peptide or a toxin based linker, preferably a peptide covalently bound to polyethylene glycol (PEG) and, wherein the adapter molecule is a double stranded RNA binding protein (DRBP) or a variant thereof.

8. The conjugate of claim 4, wherein the linker molecule comprises
(i) at least one branch point, preferably a lysine side chain, a cysteine side chain, or an unnatural amino acid containing an aminooxy moiety on the side chain, and/or
(ii) at least one cleavage site, preferably a furin or a calpain cleavage site.

9. The conjugate of claim 8, wherein the cleavage site is between module (a) and module (c) or between module (a) and compound (d).

10. The conjugate of claim 8, wherein the compound is covalently linked to the branch point, preferably via an amide-linkage to the lysine side chain, via a disulfide-linkage to the cysteine side chain or via an unnatural amino acid containing an aminooxy moiety on the side chain.

11. The conjugate of claim 8, wherein the compound is non-covalently linked to the branch point via an ionic linkage or via a hydrophobic linkage to DRBD or a variant thereof that is covalently linked via a disulfide linkage to the cysteine side chain.

12. The conjugate of claim 1, wherein
(i) the module (a) comprises a cell surface receptor ligand, an antibody, a sugar, a lipid or a nanoparticle,
(ii) the module (b) comprises an oligopeptide comprising one or more of an amino acid sequence X₁X₂X₃X₄ (SEQ ID NO: 5), wherein
X₁ is E, H, K, N, P, Q, R, or S, preferably K or R,
X₂ is D, E, A, T, V, G, S, or N, preferably D, or E,
X₃ is E, or D, preferably E,
X₄ is L, or F, preferably L, and wherein optionally the N-terminus and/or C-terminus comprises 1 to 3 additional amino acid residues;
(iii) the module (c) comprises
(a) a peptide of a protein selected from the group consisting of COX2, IgM(μ), Sgk1, MATalpha2, MF(alpha)1, CPY, a toxin A subunit, AChE, a fragment thereof, or a variant thereof, or
(b) an amino acid sequence comprising CL1 (SEQ ID NO: 31), CL2 (SEQ ID NO: 32), CL6 (SEQ ID NO: 33), CL9 (SEQ ID NO: 34), CL10 (SEQ ID NO: 35), CL11 (SEQ ID NO: 36), CL12 (SEQ ID NO: 37), CL15 (SEQ ID NO: 38), CL16 (SEQ ID NO: 39) or SL17 (SEQ ID NO: 40), and
(iv) the compound (d) comprises a nucleic acid or a peptide.

13. The conjugate of claim 12, wherein
(i) the cell surface receptor ligand is selected from the group consisting of a growth factor, a lipoprotein, a transferrin, an AMF, a surface binding lectin, a galectin, a c-type lectin, a toxin, a fragment thereof, and a variant thereof,
(ii) the antibody is selected from the group consisting of anti-TGN38/46, anti-transferrin receptor, and anti-growth factor receptor,
(iii) the lipid is selected from the group consisting of a phospholipid, a glycolipid, a sphingolipid, and a sterol lipid, and
(iv) the nanoparticle is selected from the group consisting of a metal, a silicate, and a polymer.

14. The conjugate of claim 13, wherein the cell surface receptor ligand is a toxin selected from the group consisting of B subunit of Ricin, B subunit of Abrin, B subunit of Modeccin, B subunit of Volkensin, B subunit of Cholera toxin, B subunit of Shiga toxin, B subunit of Verotoxin, domains I, II and IV of Pseudomonas Exotoxin A, and B subunit of Escherichia coli heat-labile enterotoxin.

15. The conjugate of claim 13, wherein the module (c) is selected from the from the group consisting of
(i) NX₁SX₂X₃X₄X₅X₆X₇X₈X₉INPTX₁₀X₁₁X₁₂X₁₃ (SEQ ID NO: 45), wherein X₁ is A, S, or V; X₂ is S, A, or T; X₃ is S, or V; X₄ is R, H, or N; X₅ is S, or T; X₆ is G, R, T, or A; X₇ is L, V, or M; X₈ is D, N, or E; X₉ is D, or N; X₁₀ is V, or L; X₁₁ is L, or V; X₁₂ is L, or I; and X₁₃ is K, or N;
(ii) GKPTLYX₁VSLX₂MSDTX₃GTX₄Y (SEQ ID NO: 57), wherein X₁ is N, or Q; X₂ is I, or V; X₃ is G, or A; and X₄ is C, or S;
(iii) MTX₁X₂X₃X₄EX₅X₆X₇X₈X₉X₁₀X₁₁LTYSX₁₂X_{D}RGX₁₄VAX₁₅LX₁₆AFMKQR X₁₇MGLNDFIQKX₁₈X₁₉X₂₀NX₂₁YACKHX₂₂EVQSX₂₃LX₂₄X₂₅ (SEQ ID NO: 67), wherein X₁ is V, or I; X₂ is K, or Q; X₃ is A, or T; X₄ is X (X is zero amino acid) or A; X₅ is A, or T; X₆ is A, or S; X₇ is R, K, G, or V; X₈ is S, G, or P; X₉ is T, P, or A; X₁₀ is X or P; X₁ᵢ is X or D; X₁₂ is R, or K; X₁₃ is M, or T; X₁₄ is M, or L; X₁₅ is I, or N; X₁₆ is I, or S; X₁₇ is R, or K; X₁₈ is I, or L; X₁₉ is A, or S; X₂₀ is S, N, A, or T; X₂₁ is T, or S; X₂₂ is A, P, or T; X₂₃ is I, or Y; X₂₄ is K, or N; and X₂₅ is M, I, or L;
(iv) MRFPSIFTAVLFAASSALAAPVX₁TTTEDETAQIPAEAVIGYLDLEGDFDVA VLPFSX₁STNNGLLFIX₁TTIASIAAKEEGVSLDKREAEAWHWLQLKPGQP MYKREAEAEAWHWLQLKPGQPMYKREADAEAWHWLQLKPGQPMYKR EADAEAWHWLQLKPGQPMY (SEQ ID NO: 87), wherein X₁ is N, or Q;
(v) MNKIPIKDLLNPQITDEFKSSILDINKKLFSICCNLPKLPES VTTEEEVELRDILX₁FLSRAN (SEQ ID NO: 81), wherein X₁ is G, V, or L;
(vi) DTLDEAERQWRAEFHRWSSYMVHWKNQFDHYSKQERX₁SDL (SEQ ID NO: XXX, wherein X₁ is C, or S; and
(vii) ETIDEAERQWKTEFHRWSX₁YX₂MHWKNQFDQYSRHENX₃AEL (SEQ ID NO: XXX), wherein X₁ is C, or S; X₂ is C, or M; X₃ is C, or S.

16. The conjugate of claim 15, wherein module (c) is
(i) NASSSRSGLDDINPTVLLK (SEQ ID NO: 43);
(ii) NASASHSRLDDINPTVLIK (SEQ ID NO: 46);
(iii) NASSSHSGLDDINPTVLLK (SEQ ID NO: 47);
(iv) GKPTLYNVSLIMSDTGGTCY (SEQ ID NO: 51);
(v) GKPTLYNVSLVMSDTAGTCY (SEQ ID NO: 52);
(vi) GKPTLYQVSLIMSDTGGTCY (SEQ ID NO: 53);
(vii) GKPTLYQVSLIMSDTGGTSY (SEQ ID NO: 54);
(viii) MTVKAEAARSTLTYSRMRGMVAILIAFMKQRRMGLNDFIQKIASNTYAC KHAEVQSILKM (SEQ ID NO: 60);
(ix) MTVKTEAAKGTLTYSRMRGMVAILIAFMKQRRMGLNDFIQKIANNSYAC KHPEVQSILKI (SEQ ID NO: 64);
(x) MNKIPIKDLLNPQITDEFKSSILDINKKLFSICCNLPKLPESVTTEEEVELRDI LGFLSRAN (SEQ ID NO: 79);
(xi) MNKIPIKDLLNPQITDEFKSSILDINKKLFSICCNLPKLPESVTTEEEVELRDI LVFLSRAN (SEQ ID NO: 82);
(xii) MNKIPIKDLLNPQITDEFKSSILDINKKLFSICCNLPKLPESVTTEEEVELRDI LLFLSRAN (SEQ ID NO: 83);
(xiii) DTLDEAERQWKAEFHRWSSYMVHWKNQFDHYSKQERCSDL (SEQ ID NO: 280);
(xiv) DTLDEAERQWKAEFHRWSSYMVHWKNQFDHYSKQERSSDL (SEQ ID NO: 281);
(xv) ETIDEAERQWKTEFHRWSSYMMHWKNQFDQYSRHENCAEL (SEQ ID NO: 282);
(xvi) ETIDEAERQWKTEFHRWSSYMMHWKNQFDQYSRHENSAEL (SEQ ID NO: 283);
(xvii) ETIDEAERQWKTEFHRWSCYMMHWKNQFDQYSRHENCAEL (SEQ ID NO: 284);
(xviii) ETIDEAERQWKTEFHRWSCYMMHWKNQFDQYSRHENSAEL (SEQ ID NO: 285);
(xix) ETIDEAERQWKTEFHRWSSYCMHWKNQFDQYSRHENCAEL (SEQ ID NO: 286);
(xx) ETIDEAERQWKTEFHRWSSYCMHWKNQFDQYSRHENSAEL (SEQ ID NO: 287);
(xxi) ETIDEAERQWKTEFHRWSCYCMHWKNQFDQYSRHENCAEL (SEQ ID NO: 288);
(xxii) ETIDEAERQWKTEFHRWSCYCMHWKNQFDQYSRHENSAEL (SEQ ID NO: 289);
(xxiii) DTLDEAERQWRAEFHRWSSYMVHWKNQFDHYSKQERX₁SDL, wherein X₁ is C or S (SEQ ID NO: 290); or
(xxiv) ETIDEAERQWKTEFHRWSX₁YX₂MHWKNQFDQYSRHENX₃AEL, wherein X₁ is C or S; X₂ is C or M; X₃ is C or S (SEQ ID NO: 291).

17. The conjugate of claim 16, wherein module (c) is
(SEQ ID NO: 72)

| | |
|---|---|
| (i) | MRGMVAILIAFMKQRRMGLNDFIQKIASNTYACKHAEVQSILKM; |

(SEQ ID NO: 73)

| | |
|---|---|
| (ii) | MRGMVAILIAFMKQ; |

(SEQ ID NO: 74)

| | |
|---|---|
| (iii) | GMVAILIAF; |

(SEQ ID NO: 77)

| | |
|---|---|
| (iv) | MRGMVAILIAFMKQRRMGLNDFIQKIANNSYACKHPEVQSILKI; |

(SEQ ID NO: 84)

| | |
|---|---|
| (v) | ITDEFKSSILDINKKLFSI; |
| or | |

(SEQ ID NO: 85)

| | |
|---|---|
| (vi) | ITDEFKSSILDINKKLFSICCNLPKLPESV. |

18. The conjugate of claim 12, wherein the nucleic acid is a single stranded DNA, a double stranded DNA, a single stranded RNA, a double stranded RNA, an siRNA, a transfer RNA (tRNA), a messenger RNA (mRNA), a micro RNA (miRNA), a small nuclear RNA (snRNA), a small hairpin RNA (shRNA) or a morpholino-modified iRNA.

19. The conjugate of claim 12, wherein the nucleic acid is chemically modified.

20. A conjugate according to 1 for use as a pharmaceutical.

21. A pharmaceutical composition comprising
(i) a conjugate according to claim 1, and
(ii) a pharmaceutically acceptable excipient, carrier and/or diluent.

22. A method of delivering a compound (d) to a cell comprising the steps of
(a) providing a cell,
(b) contacting a conjugate according to claim 1 comprising the compound (d) with said cell under conditions whereby the conjugate is internalized by the cell, thereby delivering the compound (d) to the cell.

23. The method according to claim 23, wherein the cell is a eukaryotic cell, an invertebrate cell, a vertebrate cell, a nematode cell, a fungal cell, an Aspergillus cell, a yeast cell, a Sacchromyces cell, a Pichia cell, an insect cell, an Sf9 cell, an animal cell, a non-human animal cell, a mammalian cell, a non-human mammalian cell, a CHO, a primate cell, a non-human primate cell, a human cell, or a plant cell.

24. A method of delivering a compound (d) to a patient comprising the step of administering a sufficient amount of a conjugate according to claim 1 to a patient, thereby delivering the compound (d) to the patient.

25. A method of modifying gene expression in a cell comprising the steps of
(a) providing a cell, and
(b) contacting the conjugate according to claim 1 comprising a compound (d) with said cell under conditions whereby the conjugate is internalized by the cell and the compound (d) of the conjugate is delivered to the cell's cytosol or nucleus, wherein the compound (d) is a nucleic acid or a peptide capable of modifying gene expression in the cell, and
(c) upon reaching the cell's cytosol or nucleus, the compound (d) modifies gene expression in the cell.

26. A method of preparing a conjugate comprising coupling at least one module (a) that mediates cell targeting and facilitates cellular uptake, at least one module (b) that facilitates transport to the endoplasmic reticulum (ER), at least one module (c) that mediates translocation from the ER to the cytosol, and at least one compound (d), wherein the modules (a), (b) and (c) and the compound (d) are linked to each other in any arrangement and in any stoichiometry.

27. A kit comprising a component to prepare the conjugate according to claim 1, wherein the kit comprises a module (a), a module (b), a module (c), and/or a compound (d) and wherein the kit comprises an optional peptide linker and/or an optional peptide comprising a cleavage site.

28. A kit comprising a delivery system comprising the conjugate according to claim 1.


SEQUENCE LISTING (311 sequences)

SEQ ID NO: 1 - protein, 267 aa, Ricinus communis
  DOMAIN (1)..(266): Ricin A chain without signal and linker peptide
IFPKQYPIINFTTAGATVQSYTNFIRAVRGRLTTGADVRHEIPVLPNRVGLPINQRFILVELSNHAELSVTLALDVTNAYVVGYRAGNSAYFFHPDNQEDAEAITHLFTDVQNRYTFAFGGNYDRLEQLAGNLRENIELGNGPLEEAISALYYYSTGGTQLPTLARSFIICIQMISEAARFQYIEGEMRTRIRYNRRSAPDPSVITLENSWGRLSTAIQESNQGAFASPIQLQRRNGSKFSVYDVSILIPIIALMVYRCAPPPSSQF

SEQ ID NO: 2 - protein, 25 aa, Homo sapiens
  DOMAIN (1)..(25): corresponding to amino acids 580 through 604 of human COX2
NASSSRSGLDDINPTVLLKERSTEL

SEQ ID NO: 3 - protein, 24 aa, Artificial Sequence
  Cx2a module (b) + (c)
NASSSRSGLDDINPTVLLKAKDEL

SEQ ID NO: 4 - protein, 24 aa, Artificial Sequence
  IgM(mu) peptide +  KDEL peptide
GKPTLYQVSLIMSDTGGTSYKDEL

SEQ ID NO: 5 - protein, 4 aa, Artificial Sequence
  variable peptide based on aa′s 1-4 of EDEL
  VARIANT (1)..(4): Xaa at 1 is E, H, K, N, P, Q, R or S; Xaa at 2 is D, E, A, T, V, G, S or N; Xaa at 3 is E or D; Xaa at 4 is L or F; and optionally the N-terminus and/or C-terminus comprises 1 to 3 additional amino acid residues
XXXX

SEQ ID NO: 6 - protein, 4 aa, Homo sapiens
  DOMAIN (1)..(4): EDEL motif
EDEL

SEQ ID NO: 7 - protein, 4 aa, Homo sapiens
  DOMAIN (1)..(4): HDEL motif
HDEL

SEQ ID NO: 8 - protein, 4 aa, Homo sapiens
  DOMAIN (1)..(4): HEEL motif
HEEL

SEQ ID NO: 9 - protein, 4 aa, Homo sapiens
  DOMAIN (1)..(4): KAEL motif
KAEL

SEQ ID NO: 10 - protein, 4 aa, Homo sapiens
  DOMAIN (1)..(4): KDEF motif
KDEF

SEQ ID NO: 11 - protein, 4 aa, Homo sapiens
  DOMAIN (1)..(4): KEDL motif
KEDL

SEQ ID NO: 12 - protein, 4 aa, Homo sapiens
  DOMAIN (1)..(4): KEEL motif
KEEL

SEQ ID NO: 13 - protein, 4 aa, Homo sapiens
  DOMAIN (1)..(4): KTEL motif
KTEL

SEQ ID NO: 14 - protein, 4 aa, Homo sapiens
  DOMAIN (1)..(4): KVEL motif
KVEL

SEQ ID NO: 15 - protein, 4 aa, Homo sapiens
  DOMAIN (1)..(4): NEDL motif
NEDL

SEQ ID NO: 16 - protein, 4 aa, Homo sapiens
  DOMAIN (1)..(4): PDEL motif
PDEL

SEQ ID NO: 17 - protein, 4 aa, Homo sapiens
  DOMAIN (1)..(4): PGEL motif
PGEL

SEQ ID NO: 18 - protein, 4 aa, Homo sapiens
  DOMAIN (1)..(4): QEDL motif
QEDL

SEQ ID NO: 19 - protein, 4 aa, Homo sapiens
  DOMAIN (1)..(4): QSEL motif
QSEL

SEQ ID NO: 20 - protein, 4 aa, Homo sapiens
  DOMAIN (1)..(4): REDL motif
REDL

SEQ ID NO: 21 - protein, 4 aa, Homo sapiens
  DOMAIN (1)..(4): RNEL motif
RNEL

SEQ ID NO: 22 - protein, 4 aa, Homo sapiens
  DOMAIN (1)..(4): RTDL motif
RTDL

SEQ ID NO: 23 - protein, 4 aa, Homo sapiens
  DOMAIN (1)..(4): RTEL motif
RTEL

SEQ ID NO: 24 - protein, 6 aa, Homo sapiens
  DOMAIN (1)..(6): ERSTEL motif
ERSTEL

SEQ ID NO: 25 - protein, 4 aa, Homo sapiens
  DOMAIN (1)..(4): KDEL motif
KDEL

SEQ ID NO: 26 - protein, 5 aa, Homo sapiens
  DOMAIN (1)..(5): AKDEL motif
AKDEL

SEQ ID NO: 27 - protein, 4 aa, Homo sapiens
  DOMAIN (1)..(4): PTEL motif
PTEL

SEQ ID NO: 28 - protein, 4 aa, Homo sapiens
  DOMAIN (1)..(4): STEL motif
STEL

SEQ ID NO: 29 - protein, 5 aa, Homo sapiens
  DOMAIN (1)..(5): REDLK motif
REDLK

SEQ ID NO: 30 - protein, 4 aa, Homo sapiens
  DOMAIN (1)..(4): RDEL motif
RDEL

SEQ ID NO: 31 - protein, 16 aa, Artificial Sequence
  CL1 deduced C-terminal destablizing seq found in screen
ACKNWFSSLSHFVIHL

SEQ ID NO: 32 - protein, 35 aa, Artificial Sequence
  CL2 deduced C-terminal destablizing seq found in screen
SLISLPLPTRVKFSSLLLIRIMKIITMTFPKKLRS

SEQ ID NO: 33 - protein, 16 aa, Artificial Sequence
  CL6 deduced C-terminal destablizing seq found in screen
FYYPIWFARVLLVHYQ

SEQ ID NO: 34 - protein, 46 aa, Artificial Sequence
  CL9 deduced C-terminal destablizing seq found in screen
SNPFSSLFGASLLIDSVSLKSNWDTSSSSCLISFFSSVMFSSTTRS

SEQ ID NO: 35 - protein, 39 aa, Artificial Sequence
  CL10 deduced C-terminal destablizing seq found in screen
CRQRFSCHLTASYPQSTVTPFLAFLRRDFFFLRHNSSAD

SEQ ID NO: 36 - protein, 46 aa, Artificial Sequence
  CL11 deduced C-terminal destablizing seq found in screen
GAPHVVLFDFELRITNPLSHIQSVSLQITLIFCSLPSLILSKFLQV

SEQ ID NO: 37 - protein, 39 aa, Artificial Sequence
  CL12 deduced C-terminal destablizing seq found in screen
NTPLFSKSFSTTCGVAKKTLLLAQISSLFFLLLSSNIAV

SEQ ID NO: 38 - protein, 45 aa, Artificial Sequence
  CL15 deduced C-terminal destablizing seq found in screen
PTVKNSPKIFCLSSSPYLAFNLEYLSLRIFSTLSKCSNTLLTSLS

SEQ ID NO: 39 - protein, 30 aa, Artificial Sequence
  CL16 deduced C-terminal destablizing seq found in screen
SNQLKRLWLWLLEVRSFDRTLRRPWIHLPS

SEQ ID NO: 40 - protein, 50 aa, Artificial Sequence
  SL17 deduced C-terminal destablizing seq found in screen
SISFVIRSHASIRMGASNDFFHKLYFTKCLTSVILSKFLIHLLLRSTPRV

SEQ ID NO: 41 - protein, 604 aa, Homo sapiens
  DOMAIN (1)..(604): 1-604 COX2 protein
MLARALLLCAVLALSHTANPCCSHPCQNRGVCMSVGFDQYKCDCTRTGFYGENCSTPEFLTRIKLFLKPTPNTVHYILTHFKGFWNVVNNIPFLRNAIMSYVLTSRSHLIDSPPTYNADYGYKSWEAFSNLSYYTRALPPVPDDCPTPLGVKGKKQLPDSNEIVEKLLLRRKFIPDPQGSNMMFAFFAQHFTHQFFKTDHKRGPAFTNGLGHGVDLNHIYGETLARQRKLRLFKDGKMKYQIIDGEMYPPTVKDTQAEMIYPPQVPEHLRFAVGQEVFGLVPGLMMYATIWLREHNRVCDVLKQEHPEWGDEQLFQTSRLILIGETIKIVIEDYVQHLSGYHFKLKFDPELLFNKQFQYQNRIAAEFNTLYHWHPLLPDTFQIHDQKYNYQQFIYNNSILLEHGITQFVESFTRQIAGRVAGGRNVPPAVQKVSQASIDQSRQMKYQSFNEYRKRFMLKPYESFEELTGEKEMSAELEALYGDIDAVELYPALLVEKPRPDAIFGETMVEVGAPFSLKGLMGNVICSPAYWKPSTFGGEVGFQIINTASIQSLICNNVKGCPFTSFSVPDPELIKTVTINASSSRSGLDDINPTVLLKERSTEL

SEQ ID NO: 42 - protein, 101 aa, Homo sapiens
  DOMAIN (1)..(101): 504-604 COX2 peptide
FGETMVEVGAPFSLKGLMGNVICSPAYWKPSTFGGEVGFQIINTASIQSLICNNVKGCPFTSFSVPDPELIKTVTINASSSRSGLDDINPTVLLKERSTEL

SEQ ID NO: 43 - protein, 19 aa, Homo sapiens
  DOMAIN (1)..(19): 580-598 COX2 peptide
NASSSRSGLDDINPTVLLK

SEQ ID NO: 44 - protein, 25 aa, Homo sapiens
NASSSRSGLDDINPTVLLKERSTEL

SEQ ID NO: 45 - protein, 19 aa, Artificial Sequence
  variable peptide based on aa′s 580-598 of COX2 protein
  VARIANT (2)..(19): 2:Xaa is A, S or V; 4:Xaa is S, A or T; 5:Xaa is S or V;  6:Xaa is R, H or N;  7:Xaa is S or T; 8: Xaa is G, R, T or A; 9:Xaa is L, V or M; 10: Xaa is D, N or E; 11:Xaa is D or N; 16:Xaa is V or L; 17:Xaa is L or V; 18:Xaa is L or I; 19:Xaa is K or N
NXSXXXXXXXXINPTXXXX

SEQ ID NO: 46 - protein, 19 aa, Mus musculus
  VARIANT (1)..(19): 580-598 COX2 peptide variant
NASASHSRLDDINPTVLIK

SEQ ID NO: 47 - protein, 19 aa, Felis catus
  VARIANT (1)..(19): 580-598 COX2 peptide variant
NASSSHSGLDDINPTVLLK

SEQ ID NO: 48 - protein, 19 aa, Artificial Sequence
  variable peptide based on aa′s 580-598 of COX2 protein
  VARIANT (2)..(9): 2: Xaa is A, G or V; 5: Xaa is S or A; 6: Xaa is R, H or N; 8: Xaa is G, R or A; 9: Xaa is L or S
NXSSXXSXXDDINPTVLLK

SEQ ID NO: 49 - protein, 455 aa, Mus musculus
  DOMAIN (1)..(455): aa 1-455 of murine IgM(mu)
ESQSFPNVFPLVSCESPLSDKNLVAMGCLARDFLPSTISFTWNYQNNTEVIQGIRTFPTLRTGGKYLATSQVLLSPKSILEGSDEYLVCKIHYGGKNKDLHVPIPAVAEMNPNVNVFVPPRDGFSGPAPRKSKLICEATNFTPKPITVSWLKDGKLVESGFTTDPVTIENKGSTPQTYKVISTLTISEIDWLNLNVYTCRVDHRGLTFLKNVSSTCAASPSTDILTFTIPPSFADIFLSKSANLTCLVSNLATYETLNISWASQSGEPLETKIKIMESHPNGTFSAKGVASVCVEDWNNRKEFVCTVTHRDLPSPQKKFISKPNEVHKHPPAVYLLPPAREQLNLRESATVTCLVKGFSPADISVQWLQRGQLLPQEKYVTSAPMPEPGAPGFYFTHSILTVTEEEWNSGETYTCVVGHEALPHLVTERTVDKSTGKPTLYNVSLIMSDTGGTCY

SEQ ID NO: 50 - protein, 35 aa, Mus musculus
  DOMAIN (1)..(35): aa 421-455 of murine IgM(mu)
ALPHLVTERTVDKSTGKPTLYNVSLIMSDTGGTCY

SEQ ID NO: 51 - protein, 20 aa, Mus musculus
  DOMAIN (1)..(20): aa 436-455 of murine IgM (mu)
GKPTLYNVSLIMSDTGGTCY

SEQ ID NO: 52 - protein, 20 aa, Homo sapiens
  VARIANT (1)..(20): human peptide variant of aa 436-455 of murine IgM(mu)
GKPTLYNVSLVMSDTAGTCY

SEQ ID NO: 53 - protein, 20 aa, Rattus norvegicus
  VARIANT (1)..(20): rat peptide variant I of aa 436-455 of murine IgM(mu)
GKPTLYQVSLIMSDTGGTCY

SEQ ID NO: 54 - protein, 20 aa, Rattus norvegicus
  VARIANT (1)..(20): rat peptide variant II of aa 436-455 of murine IgM(mu)
GKPTLYQVSLIMSDTGGTSY

SEQ ID NO: 55 - protein, 453 aa, Homo sapiens
  DOMAIN (1)..(453): human IgM(mu)
GSLSAPTLFPLVSCENSPSDTSSVAVGCLAQDFLPDSITFSWKYKNNSDISSTRGFPSVLRGGKHAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGTTTYKVTSTLTIKESDWLSQSMFTCRVDHRGLTFQQNASSMCGPDQDTAIRVFSIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 56 - protein, 35 aa, Homo sapiens
  DOMAIN (1)..(35): aa 421-455 of human IgM(mu)
ALPNRVTERTVDKSTGKPTLYNVSLVMSDTAGTCY

SEQ ID NO: 57 - protein, 20 aa, Artificial Sequence
  artificial IgM(mu) peptide
  VARIANT (7)..(19): 7: Xaa is N or Q; 11: Xaa is I or V; 16:  Xaa is G or A; 19: Xaa is C or S
GKPTLYXVSLXMSDTXGTXY

SEQ ID NO: 58 - protein, 431 aa, Mus musculus
  DOMAIN (1)..(431): aa 1-431 of murine Sgk1
MTVKAEAARSTLTYSRMRGMVAILIAFMKQRRMGLNDFIQKIASNTYACKHAEVQSILKMSHPQEPELMNANPSPPPSPSQQINLGPSSNPHAKPSDFHFLKVIGKGSFGKVLLARHKAEEVFYAVKVLQKKAILKKKEEKHIMSERNVLLKNVKHPFLVGLHFSFQTADKLYFVLDYINGGELFYHLQRERCFLEPRARFYAAEIASALGYLHSLNIVYRDLKPENILLDSQGHIVLTDFGLCKENIEHNGTTSTFCGTPEYLAPEVLHKQPYDRTVDWWCLGAVLYEMLYGLPPFYSRNTAEMYDNILNKPLQLKPNITNSARHLLEGLLQKDRTKRLGAKDDFMEIKSHIFFSLINWDDLINKKITPPFNPNVSGPSDLRHFDPEFTEEPVPSSIGRSPDSILVTASVKEAAEAFLGFSYAPPVDSFL

SEQ ID NO: 59 - protein, 100 aa, Mus musculus
  DOMAIN (1)..(100): aa 1-100 of murine Sgk1
MTVKAEAARSTLTYSRMRGMVAILIAFMKQRRMGLNDFIQKIASNTYACKHAEVQSILKMSHPQEPELMNANPSPPPSPSQQINLGPSSNPHAKPSDFHF

SEQ ID NO: 60 - protein, 60 aa, Mus musculus
  DOMAIN (1)..(60): aa 1-60 of murine Sgk1
MTVKAEAARSTLTYSRMRGMVAILIAFMKQRRMGLNDFIQKIASNTYACKHAEVQSILKM

SEQ ID NO: 61 - protein, 33 aa, Mus musculus
  DOMAIN (1)..(33): aa 1-33 of murine Sgk1
MTVKAEAARSTLTYSRMRGMVAILIAFMKQRRM

SEQ ID NO: 62 - protein, 431 aa, Homo sapiens
  DOMAIN (1)..(431): human Sgk1
MTVKTEAAKGTLTYSRMRGMVAILIAFMKQRRMGLNDFIQKIANNSYACKHPEVQSILKISQPQEPELMNANPSPPPSPSQQINLGPSSNPHAKPSDFHFLKVIGKGSFGKVLLARHKAEEVFYAVKVLQKKAILKKKEEKHIMSERNVLLKNVKHPFLVGLHFSFQTADKLYFVLDYINGGELFYHLQRERCFLEPRARFYAAEIASALGYLHSLNIVYRDLKPENILLDSQGHIVLTDFGLCKENIEHNSTTSTFCGTPEYLAPEVLHKQPYDRTVDWWCLGAVLYEMLYGLPPFYSRNTAEMYDNILNKPLQLKPNITNSARHLLEGLLQKDRTKRLGAKDDFMEIKSHVFFSLINWDDLINKKITPPFNPNVSGPNDLRHFDPEFTEEPVPNSIGKSPDSVLVTASVKEAAEAFLGFSYAPPTDSFL

SEQ ID NO: 63 - protein, 100 aa, Homo sapiens
  DOMAIN (1)..(100)
  aa 1-100 of human Sgk1
MTVKTEAAKGTLTYSRMRGMVAILIAFMKQRRMGLNDFIQKIANNSYACKHPEVQSILKISQPQEPELMNANPSPPPSPSQQINLGPSSNPHAKPSDFHF

SEQ ID NO: 64 - protein, 60 aa, Homo sapiens
  DOMAIN (1)..(60): amino acids 1 through 60 of human Sgk1
MTVKTEAAKGTLTYSRMRGMVAILIAFMKQRRMGLNDFIQKIANNSYACKHPEVQSILKI

SEQ ID NO: 65 - protein, 33 aa, Homo sapiens
  DOMAIN (1)..(33): aa 1-33 of human Sgk1
MTVKTEAAKGTLTYSRMRGMVAILIAFMKQRRM

SEQ ID NO: 66 - protein, 30 aa, Homo sapiens
  DOMAIN (1)..(30): 1-30 of human Sgk1 protein
MTVKTEAAKGTLTYSRMRGMVAILIAFMKQ

SEQ ID NO: 67 - protein, 63 aa, Artificial Sequence
  variable peptide based on aa 1-60 of murine Sgk1
  VARIANT (3)..(14): 3: Xaa is V or I; 4: Xaa is K or Q; 5: Xaa is A or T; 6: Xaa is zero (0) aa or A; 8: Xaa is A or T; 9: Xaa is A or S; 10: Xaa is R, K, G or V; 11: Xaa is S, G or P; 12: Xaa is T, P or A; 13: Xaa is X or P; 14: Xaa is X or D;
  VARIANT (19)..(60): 19: Xaa is R or K; 20: Xaa is M or T; 23: Xaa is M or L; 26: Xaa is I or N; 28: Xaa is I or S; 35: Xaa is R or K; 45: Xaa is I or L; 46: Xaa is A or S; 47: Xaa is S, N, A or T; 49: Xaa is T or S; 55: Xaa is A, P or T; 60: Xaa is I or Y;
  VARIANT (62)..(63): 62: Xaa is K or N; and 63: Xaa is M, I or L
MTXXXXEXXXXXXXLTYSXXRGXVAXLXAFMKQRXMGLNDFIQKXXXNXYACKHXEVQSXLXX

SEQ ID NO: 68 - protein, 60 aa, Rattus norvegicus
  VARIANT (1)..(60): rat peptide variant of aa 1-60 of murine Sgk1
MTVKTEAARSTLTYSRMRGMVAILIAFMKQRRMGLNDFIQKLANNSYACKHPEVQSYLKI

SEQ ID NO: 69 - protein, 60 aa, Oryctolagus cuniculus
  VARIANT (1)..(60): rabbit peptide variant of aa 1-60 of murine Sgk1
MTVKTEAARGPLTYSRMRGMVAILIAFMKQRRMGLNDFIQKIANNSYACKHTEVQSILKI

SEQ ID NO: 70 - protein, 60 aa, Gallus gallus
  VARIANT (1)..(60): chicken peptide variant of aa 1-60 of murine Sgk1
MTVKAAEASGPALTYSKMRGMVAILIAFMKQRRMGLNDFIQKIATNSYACKHPEVQSILK

SEQ ID NO: 71 - protein, 60 aa, Danio rerio
  VARIANT (1)..(60)
  zebrafish peptide variant of aa 1-60 of murine Sgk1
MTIQTETSVSAPDLTYSKTRGLVANLSAFMKQRKMGLNDFIQKLSANSYACKHPEVQSIL

SEQ ID NO: 72 - protein, 44 aa, Mus musculus
  DOMAIN (1)..(44)
  aa 17-60 of murine Sgk1
MRGMVAILIAFMKQRRMGLNDFIQKIASNTYACKHAEVQSILKM

SEQ ID NO: 73 - protein, 14 aa, Mus musculus
  DOMAIN (1)..(14)
  aa 17-30 of murine Sgk1
MRGMVAILIAFMKQ

SEQ ID NO: 74 - protein, 9 aa, Mus musculus
  DOMAIN (1)..(9): aa 19-27 of murine Sgk1
GMVAILIAF

SEQ ID NO: 75 - protein, 17 aa, Mus musculus
  DOMAIN (1)..(17)
  aa 17-33 of murine Sgk1
MRGMVAILIAFMKQRRM

SEQ ID NO: 76 - protein, 7 aa, Mus musculus
  DOMAIN (1)..(7): aa 19-25 of murine Sgk1
GMVAILI

SEQ ID NO: 77 - protein, 44 aa, Homo sapiens
  DOMAIN (1)..(44): aa 17-60 of human Sgk1
MRGMVAILIAFMKQRRMGLNDFIQKIANNSYACKHPEVQSILKI

SEQ ID NO: 78 - protein, 210 aa, Saccharomyces cerevisiae
  DOMAIN (1)..(210): aa 1-210 of MAT(alpha)2
MNKIPIKDLLNPQITDEFKSSILDINKKLFSICCNLPKLPESVTTEEEVELRDILGFLSRANKNRKISDEEKKLLQTTSQLTTTITVLLKEMRSIENDRSNYQLTQKNKSADGLVFNVVTQDMINKSTKPYRGHRFTKENVRILESWFAKNIENPYLDTKGLENLMKNTSLSRIQIKNWVSNRRRKEKTITIAPELADLLSGEPLAKKKE

SEQ ID NO: 79 - protein, 100 aa, Saccharomyces cerevisiae
  DOMAIN (1)..(100): aa 1-100 of MAT(alpha)2
MNKIPIKDLLNPQITDEFKSSILDINKKLFSICCNLPKLPESVTTEEEVELRDILGFLSRANKNRKISDEEKKLLQTTSQLTTTITVLLKEMRSIENDRS

SEQ ID NO: 80 - protein, 62 aa, Saccharomyces cerevisiae
  DOMAIN (1)..(62): aa 1-62 of MAT(alpha)2
MNKIPIKDLLNPQITDEFKSSILDINKKLFSICCNLPKLPESVTTEEEVELRDILGFLSRAN

SEQ ID NO: 81 - protein, 62 aa, Artificial Sequence
  variable peptide based on aa 1-62 of MAT(alpha)2
  VARIANT (56)..(56): Xaa at 56 is G, V or L
MNKIPIKDLLNPQITDEFKSSILDINKKLFSICCNLPKLPESVTTEEEVELRDILXFLSRAN

SEQ ID NO: 82 - protein, 62 aa, Artificial Sequence
  peptide variant I based on aa 1-62 of MAT(alpha)2
MNKIPIKDLLNPQITDEFKSSILDINKKLFSICCNLPKLPESVTTEEEVELRDILVFLSRAN

SEQ ID NO: 83 - protein, 62 aa, Artificial Sequence
  peptide variant II based on aa 1-62 of MAT(alpha)2
MNKIPIKDLLNPQITDEFKSSILDINKKLFSICCNLPKLPESVTTEEEVELRDILLFLSRAN

SEQ ID NO: 84 - protein, 19 aa, Saccharomyces cerevisiae
  DOMAIN (1)..(19): Deg1 Determinant peptide of MAT(alpha)2
ITDEFKSSILDINKKLFSI

SEQ ID NO: 85 - protein, 30 aa, Saccharomyces cerevisiae
  DOMAIN (1)..(30): Deg1 + heptad repeat peptide of MAT(alpha)2
ITDEFKSSILDINKKLFSICCNLPKLPESV

SEQ ID NO: 86 - protein, 165 aa, Saccharomyces cerevisiae
  DOMAIN (1)..(165): aa 1-165 of MF(alpha)1
MRFPSIFTAVLFAASSALAAPVNTTTEDETAQIPAEAVIGYLDLEGDFDVAVLPFSNSTNNGLLFINTTIASIAAKEEGVSLDKREAEAWHWLQLKPGQPMYKREAEAEAWHWLQLKPGQPMYKREADAEAWHWLQLKPGQPMYKREADAEAWHWLQLKPGQPMY

SEQ ID NO: 87 - protein, 165 aa, Artificial Sequence
  variable peptide based on aa 1-165 of MF(alpha)1
  VARIANT (1)..(165): Xaa is N or Q
MRFPSIFTAVLFAASSALAAPVXTTTEDETAQIPAEAVIGYLDLEGDFDVAVLPFSXSTNNGLLFIXTTIASIAAKEEGVSLDKREAEAWHWLQLKPGQPMYKREAEAEAWHWLQLKPGQPMYKREADAEAWHWLQLKPGQPMYKREADAEAWHWLQLKPGQPMY

SEQ ID NO: 88 - protein, 165 aa, Saccharomyces cerevisiae
  VARIANT (1)..(165): peptide variant I based on aa 1-165 of MF(alpha)1
MRFPSIFTAVLFAASSALAAPVQTTTEDETAQIPAEAVIGYLDLEGDFDVAVLPFSQSTNNGLLFIQTTIASIAAKEEGVSLDKREAEAWHWLQLKPGQPMYKREAEAEAWHWLQLKPGQPMYKREADAEAWHWLQLKPGQPMYKREADAEAWHWLQLKPGQPMY

SEQ ID NO: 89 - protein, 165 aa, Saccharomyces cerevisiae
  VARIANT (1)..(165): peptide variant II based on aa 1-165 of MF(alpha)1
MRFPSIFTAVLFAASSALAAPVNTTTEDETAQIPAEAVIGYLDLEGDFDVAVLPFSNSTNNGLLFIQTTIASIAAKEEGVSLDKREAEAWHWLQLKPGQPMYKREAEAEAWHWLQLKPGQPMYKREADAEAWHWLQLKPGQPMYKREADAEAWHWLQLKPGQPMY

SEQ ID NO: 90 - protein, 165 aa, Saccharomyces cerevisiae
  VARIANT (1)..(165): peptide variant III based on aa 1-165 of MF(alpha)1
MRFPSIFTAVLFAASSALAAPVQTTTEDETAQIPAEAVIGYLDLEGDFDVAVLPFSNSTNNGLLFINTTIASIAAKEEGVSLDKREAEAWHWLQLKPGQPMYKREAEAEAWHWLQLKPGQPMYKREADAEAWHWLQLKPGQPMYKREADAEAWHWLQLKPGQPMY

SEQ ID NO: 91 - protein, 523 aa, Saccharomyces cerevisiae
  DOMAIN (1)..(523): aa 1-523 of CPY
MILHTYIILSLLTIFPKAIGLSLQMPMALEASYASLVEKATLAVGQEIDAIQKGIQQGWLEVETRFPTIVSQLSYSTGPKFAIKKKDATFWDFYVESQELPNYRLRVKRNNPEVLKVDFTKQYSGYLDVEADDKHFFYWFFESRNDPQNDPIILWLNGGPGCSSLTGLFFELGSSRINENLKPIFNPYSWNGNASIIYLDQPVNVGFSYSSSSVSNTVVAGEDVYAFLQLFFQHFPEYQTNDFHIAGESYAGHYIPVFADEILSQKNRNFNLTSVLIGNGLTDPLTQYRYYEPMACGEGGAPSVLPADECENMLVTQDKCLSLIQACYDSQSAFTCAPAAIYCNNAQMGPYQRTGKNVYDIRKECDGGSLCYKDLEFIDTYLNQKFVQDALGAEVDTYESCNFEINRNFLFAGDWMKPYHEHVSSLLNKGLPVLIYAGDKDFICNWLGNRAWTDVLPWVDADGFEKAEVQDWLVNGRKAGEFKNYSNFTYLRVYDAGHMAPYDQPENSHEMVNRWISGDFSFH

SEQ ID NO: 92 - protein, 5 aa, Artificial Sequence
  Peptide linker I
GGGGS

SEQ ID NO: 93 - protein, 12 aa, Artificial Sequence
  Peptide linker II
GKSSGSGSESKS

SEQ ID NO: 94 - protein, 14 aa, Artificial Sequence
  Peptide linker III
GSTSGSGKSSEGKG

SEQ ID NO: 95 - protein, 18 aa, Artificial Sequence
  Peptide linker IV
GSTSGSGKSSEGSGSTKG

SEQ ID NO: 96 - protein, 18 aa, Artificial Sequence
  Peptide linker V
GSTSGSGKPGSGEGSTKG

SEQ ID NO: 97 - protein, 14 aa, Artificial Sequence
  Peptide linker VI
EGKSSGSGSESKEF

SEQ ID NO: 98 - protein, 6 aa, Artificial Sequence
  peptide linker
SGSGSG

SEQ ID NO: 99 - protein, 41 aa, Hepatitis C virus
  DOMAIN (1)..(41): aa 20 to 60 of F protein derived from the HCV-1 isolate (genotype 1a)
HRTSSSRVAVRSLVEFTCCRAGALDWVCARRGRLPSGRNLE

SEQ ID NO: 100 - protein, 45 aa, Homo sapiens
  DOMAIN (1)..(45): N-terminal domain of thymidilate synthese
MPVAGSELPRRPLPPAAQERDAEPRPPHGELQYLGQIQHILRCGV

SEQ ID NO: 101 - protein, 37 aa, Mus musculus
  DOMAIN (1)..(37): ornithine decarboxylase degron
FPPEVEEQDDGTLPMSCAQESGMDRHPAACASARINV

SEQ ID NO: 102 - protein, 31 aa, Homo sapiens
  DOMAIN (1)..(31): degron
PTSPDRPGSTSPFAPSATDLPSMPEPALTSR

SEQ ID NO: 103 - protein, 34 aa, Saccharomyces cerevisiae
EDEDSDWDSVSNDSEFYADEDDEEYDDYNEEEAD

SEQ ID NO: 104 - protein, 70 aa, Unknown
  DRBD peptide
FFMEELNTYRQKQGVVLKYQELPNSGPPHDRRFTFQVIIDGREFPEGEGRSKKEAKNAAAKLAVEILNKE

SEQ ID NO: 105 - protein, 4 aa, Artificial Sequence
  furin Arg-X-X-Arg
  VARIANT (2)..(3): X (or Xaa, respectively) can be any amino acid
  VARIANT (2)..(3): Xaa can be any amino acid
RXXR

SEQ ID NO: 106 - protein, 4 aa, Artificial Sequence
  furin Arg-X-Lys/Arg-Arg
  VARIANT (2)..(3): Xaa at 2 can be any amino acid, Xaa at 3 can be Lys or Arg
RXXR

SEQ ID NO: 107 - protein, 11 aa, Artificial Sequence
  Peptide with cleavage DOMAIN
TPLKSPPPSPR

SEQ ID NO: 108 - protein, 558 aa, Homo sapiens
  DOMAIN (1)..(558): Full length AMF protein
MAALTRDPQFQKLQQWYREHRSELNLRRLFDANKDRFNHFSLTLNTNHGHILVDYSKNLVTEDVMRMLVDLAKSRGVEAARERMFNGEKINYTEGRAVLHVALRNRSNTPILVDGKDVMPEVNKVLDKMKSFCQRVRSGDWKGYTGKTITDVINIGIGGSDLGPLMVTEALKPYSSGGPRVWYVSNIDGTHIAKTLAQLNPESSLFIIASKTFTTQETITNAETAKEWFLQAAKDPSAVAKHFVALSTNTTKVKEFGIDPQNMFEFWDWVGGRYSLWSAIGLSIALHVGFDNFEQLLSGAHWMDQHFRTTPLEKNAPVLLALLGIWYINCFGCETHAMLPYDQYLHRFAAYFQQGDMESNGKYITKSGTRVDHQTGPIVWGEPGTNGQHAFYQLIHQGTKMIPCDFLIPVQTQHPIRKGLHHKILLANFLAQTEALMRGKSTEEARKELQAAGKSPEDLERLLPHKVFEGNRPTNSIVFTKLTPFMLGALVAMYEHKIFVQGIIWDINSFDQWGVELGKQLAKKIEPELDGSAQVTSHDASTNGLINFIKQQREARVQ

SEQ ID NO: 109 - protein, 558 aa, Mus musculus
  DOMAIN (1)..(558): Full length AMF protein
MAALTRNPQFQKLLEWHRANSANLKLRELFEADPERFNNFSLNLNTNHGHILVDYSKNLVNKEVMQMLVELAKSRGVEAARDNMFSGSKINYTENRAVLHVALRNRSNTPIKVDGKDVMPEVNRVLDKMKSFCQRVRSGDWKGYTGKSITDIINIGIGGSDLGPLMVTEALKPYSKGGPRVWFVSNIDGTHIAKTLASLSPETSLFIIASKTFTTQETITNAETAKEWFLEAAKDPSAVAKHFVALSTNTAKVKEFGIDPQNMFEFWDWVGGRYSLWSAIGLSIALHVGFDHFEQLLSGAHWMDQHFLKTPLEKNAPVLLALLGIWYINCYGCETHALLPYDQYMHRFAAYFQQGDMESNGKYITKSGARVDHQTGPIVWGEPGTNGQHAFYQLIHQGTKMIPCDFLIPVQTQHPIRKGLHHKILLANFLAQTEALMKGKLPEEARKELQAAGKSPEDLEKLLPHKVFEGNRPTNSIVFTKLTPFILGALIAMYEHKIFVQGIMWDINSFDQWGVELGKQLAKKIEPELEGSSAVTSHDSSTNGLISFIKQQRDTKLE

SEQ ID NO: 110 - protein, 374 aa, Homo sapiens
  DOMAIN (1)..(374): Human Sulfatase-modifying factor 1 (SUMF1)
MAAPALGLVCGRCPELGLVLLLLLLSLLCGAAGSQEAGTGAGAGSLAGSCGCGTPQRPGAHGSSAAAHRYSREANAPGPVPGERQLAHSKMVPIPAGVFTMGTDDPQIKQDGEAPARRVTIDAFYMDAYEVSNTEFEKFVNSTGYLTEAEKFGDSFVFEGMLSEQVKTNIQQAVAAAPWWLPVKGANWRHPEGPDSTILHRPDHPVLHVSWNDAVAYCTWAGKRLPTEAEWEYSCRGGLHNRLFPWGNKLQPKGQHYANIWQGEFPVTNTGEDGFQGTAPVDAFPPNGYGLYNIVGNAWEWTSDWWTVHHSVEETLNPKGPPSGKDRVKKGGSYMCHRSYCYRYRCAARSQNTPDSSASNLGFRCAADRLPTMD

SEQ ID NO: 111 - protein, 12 aa, Homo sapiens
  DOMAIN (1)..(12): TfR binding peptide
THRPPMWSPVWP

SEQ ID NO: 112 - protein, 9 aa, Homo sapiens
  DOMAIN (1)..(9): TfR binding peptide
GHKVKRPKG

SEQ ID NO: 113 - protein, 7 aa, Homo sapiens
  DOMAIN (1)..(7): TfR binding peptide
HAIYPRH

SEQ ID NO: 114 - protein, 613 aa, Pseudomonas aeruginosa
  DOMAIN (1)..(613): Exotoxin A
  DOMAIN (1)..(252): IA (required for cell recognition)
  DOMAIN (253)..(364): II (required for translocation in target cell cytoplasm)
  DOMAIN (365)..(404): IB
  DOMAIN (405)..(613): III (required for ADP-ribosyl activity)
AEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKPTVISHRLHFPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAAGECAGPADSGDALLERNYPTGAEFLGDGGDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 115 - protein, 262 aa, Ricinus communis
  DOMAIN (1)..(262): Ricin B chain
ADVCMDPEPIVRIVGRNGLCVDVRDGRFHNGNAIQLWPCKSNTDANQLWTLKRDNTIRSNGKCLTTYGYSPGVYVMIYDCNTAATDATRWQIWDNGTIINPRSSLVLAATSGNSGTTLTVQTNIYAVSQGWLPTNNTQPFVTTIVGLYGLCLQANSGQVWIEDCSSEKAEQQWALYADGSIRPQQNRDNCLTSDSNIRETVVKILSCGPASSGQRWMFKNDGTILNLYSGLVLDVRASDPSLKQIILYPLHGDPNQIWLPLF

SEQ ID NO: 116 - protein, 28 aa, Ricinus communis
  DOMAIN (1)..(28): aa 240-267 of ricin
FSVYDVSILIPIIALMVYRCAPPPSSQF

SEQ ID NO: 117 - protein, 103 aa, Vibrio cholerae
  DOMAIN (1)..(103): Cholera toxin B chain
TPQNITDLCAEYHNTQIYTLNDKIFSYTESLAGKREMAIITFKNGAIFQVEVPGSQHIDSQKKAIERMKDTLRIAYLTEAKVEKLCVWNNKTPHAIAAISMAN

SEQ ID NO: 118 - protein, 33 aa, Vibrio cholerae
  DOMAIN (1)..(33): aa 180-212 of cholera toxin
YGLAGFPPEHRAWREEPWIHHAPPGCGNAPRSS

SEQ ID NO: 119 - protein, 69 aa, Shigella dysenteriae
  DOMAIN (1)..(69): Stx1a B subunit
TPDCVTGKVEYTKYNDDDTFTVKVGDKELFTNRWNLQSLLLSAQITGMTVTIKTNACHNGGGFSEVIFR

SEQ ID NO: 120 - protein, 69 aa, Escherichia coli; identical to SEQ ID NO: 119
  DOMAIN (1)..(69): Stx1b subunit B

SEQ ID NO: 121 - protein, 68 aa, Escherichia coli
  DOMAIN (1)..(68): Stx1c subunit B
PDCVTGKVEYTKYNDDDTFTVKVGDKELFTNRWNLQSLLLSAQITGMTVTIKTNACHNGGGFSEVIFR

SEQ ID NO: 122 - protein, 68 aa, Escherichia coli
  DOMAIN (1)..(68): Stx1d subunit B
PDCVTGKVEYTKYNDDDTFTVKVADKELFTNRWNLQSLLLSAQITGMTVTIKTTACHNGGGFSEVIFR

SEQ ID NO: 123 - protein, 70 aa, Escherichia coli
  DOMAIN (1)..(70): Stx2a subunit B
ADCAKGKIEFSKYNEDDTFTVKVDGKEYWTSRWNLQPLLQSAQLTGMTVTIKSSTCESGSGFAEVQFNND

SEQ ID NO: 124 - protein, 68 aa, Escherichia coli
  DOMAIN (1)..(68): Stx2b subunit B
ADCAKGKIEFSKYNENDTFTVKVAGKEYWTNRWNLQPLLQSAQLTGMTVTIKSNTCASGSGFAEVQFN

SEQ ID NO: 125 - protein, 70 aa, Escherichia coli
  DOMAIN (1)..(70): Stx2c subunit B subtype ref
ADCAKGKIEFSKYNEDDTFTVKVDGKEYWTSRWNLQPLLQSAQLTGMTVTVKSSTCESGSGFAEVQFNND

SEQ ID NO: 126 - protein, 70 aa, Escherichia coli
  DOMAIN (1)..(70): Stx2c subunit B subtype variant
ADCAKGKIEFSKYNENDTFTVKVAGKEYWTSRWNLQPLLQSAQLTGMTVTIKSSTCESGSGFAEVQFNND

SEQ ID NO: 127 - protein, 70 aa, Escherichia coli; identical to SEQ ID NO: 126
  DOMAIN (1)..(70): Stx2d subunit B (subtype ref)

SEQ ID NO: 128 - protein, 70 aa, Escherichia coli
  VARIANT (1)..(70): Stx2d subunit B (subtype variant 1)
ADCAKGKIEFSKYNENDTFTVKVDGKEYWTSRWNLQPLLQSAQLTGMTVTIKSSTCASGSGFAEVQFNND

SEQ ID NO: 129 - protein, 70 aa, Escherichia coli
  VARIANT (1)..(70): Stx2d subunit B (subtype variant 2)
ADCAKGKIEFSKYNENDTFTVKVAGKEYWTSRWNLQPLLQSAQLTGMTVTIKSSTCASGSGFAEVQFNND

SEQ ID NO: 130 - protein, 103 aa, Escherichia coli
  DOMAIN (1)..(103): Heat-labile enterotoxin B chain
APQSITELCSEYHNTQIYTINDKILSYTESMAGKREMVIITFKSGATFQVEVPGSQHIDSQKKAIERMKDTLRITYLTETKIDKLCVWNNKTPNSIAAISMEN

SEQ ID NO: 131 - protein, 103 aa, Escherichia coli
  DOMAIN (1)..(103): Heat-labile enterotoxin B chain (LT-B, porcine), B chain without signal peptide
APQTITELCSEYRNTQIYTINDKILSYTESMAGKREMVIITFKSGETFQVEVPGSQHIDSQKKAIERMKDTLRITYLTETKIDKLCVWNNKTPNSIAAISMKN

SEQ ID NO: 132 - protein, 104 aa, Escherichia coli
  DOMAIN (1)..(104): Heat-labile enterotoxin IIA, B chain (LT-IIA)
QVYAGVSEHFRNICNQTTADIVAGVQLKKYIADVNTNTRGIYVVSNTGGVWYIPGGRDYPDNFLSGEIRKTAMAAILSDTKVNLCAKTSSSPNHIWAMELDRES

SEQ ID NO: 133 - protein, 99 aa, Escherichia coli
  DOMAIN (1)..(99): Heat-labile enterotoxin IIB, B chain (LT-IIB)
GASQFFKDNCNRTTASLVEGVELTKYISDINNNTDGMYVVSSTGGVWRISRAKDYPDNVMTAEMRKIAMAAVLSGMRVNMCASPASSPNVIWAIELEAE

SEQ ID NO: 134 - protein, 267 aa, Abrus precatorius
  DOMAIN (1)..(267): Abrin B chain
IVEKSKICSSRYEPTVRIGGRDGMCVDVYDNGYHNGNRIIMWKCKDRLEENQLWTLKSDKTIRSNGKCLTTYGYAPGSYVMIYDCTSAVAEATYWEIWDNGTIINPKSALVLSAESSSMGGTLTVQTNEYLMRQGWRTGNNTSPFVTSISGYSDLCMQAQGSNVWMADCDSNKKEQQWALYTDGSIRSVQNTNNCLTSKDHKQGSTILLMGCSNGWASQRWVFKNDGSIYSLYDDMVMDVKGSDPSLKQIILWPYTGKPNQIWLTLF

SEQ ID NO: 135 - protein, 528 aa, Abrus precatorius
  DOMAIN (1)..(251): Abrin-a A chain
  DOMAIN (252)..(261): Linker peptide
  DOMAIN (262)..(528): Abrin-a B chain
QDRPIKFSTEGATSQSYKQFIEALRERLRGGLIHDIPVLPDPTTLQERNRYITVELSNSDTESIEVGIDVTNAYVVAYRAGTQSYFLRDAPSSASDYLFTGTDQHSLPFYGTYGDLERWAHQSRQQIPLGLQALTHGISFFRSGGNDNEEKARTLIVIIQMVAEAARFRYISNRVRVSIQTGTAFQPDAAMISLENNWDNLSRGVQESVQDTFPNQVTLTNIRNEPVIVDSLSHPTVAVLALMLFVCNPPNANQSPLLIRSIVEKSKICSSRYEPTVRIGGRDGMCVDVYDNGYHNGNRIIMWKCKDRLEENQLWTLKSDKTIRSNGKCLTTYGYAPGSYVMIYDCTSAVAEATYWEIWDNGTIINPKSALVLSAESSSMGGTLTVQTNEYLMRQGWRTGNNTSPFVTSISGYSDLCMQAQGSNVWMADCDSNKKEQQWALYTDGSIRSVQNTNNCLTSKDHKQGSTILLMGCSNGWASQRWVFKNDGSIYSLYDDMVMDVKGSDPSLKQIILWPYTGKPNQIWLTLF

SEQ ID NO: 136 - protein, 527 aa, Abrus precatorius
  DOMAIN (1)..(250): Abrin-b A chain
  DOMAIN (251)..(260): Linker peptide
  DOMAIN (261)..(527): Abrin-b B chain
QDQVIKFTTEGATSQSYKQFIEALRQRLTGGLIHGIPVLPDPTTLQERNRYISVELSNSDTESIEAGIDVSNAYVVAYRAGNRSYFLRDAPTSASRYLFTGTQQYSLRFNGSYIDLERLARQTRQQIPLGLQALRHAISFLQSGTDDQEIARTLIVIIQMASEAARYRFISYRVGVSIRTNTAFQPDAAMISLENNWDNLSGGVQQSVQDTFPNAVTLRSVNNQPVIVDSLTHQSVAVLALMLFVCNPPNANQSPLLIRSIVEKSKICSSRYEPTVRIGGRNGMCVDVYDDGYHNGNRIIAWKCKDRLEENQLWTLKSDKTIRSNGKCLTTEGYAPGNYVMIYDCTSAVAEATYWEIWDNGTIINPKSALVLSAESSSMGGTLTVQTNEYLMRQGWRTGNNTSPFVTSISGYSDLCMQAQGSNVWLAYCDNNKKEQQWALYTDGSIRSVQNTNNCLTSKDHKQGSPIVLMACSNGWASQRWLFRNDGSIYNLHDDMVMDVKRSDPSLKEIILHPYHGKPNQIWLTLF

SEQ ID NO: 137 - protein, 562 aa, Abrus precatorius
  DOMAIN (1)..(34): signal peptide
  DOMAIN (35)..(285): Abrin-c A chain
  DOMAIN (286)..(295): Linker peptide
  DOMAIN (296)..(562): Abrin-c B chain
MDKTLKLLILCLAWTCSFSALRCAARTYPPVATNQDQVIKFTTEGATSQSYKQFIEALRQRLTGGLIHDIPVLPDPTTVEERNRYITVELSNSERESIEVGIDVTNAYVVAYRAGSQSYFLRDAPASASTYLFPGTQRYSLRFDGSYGDLERWAHQTREEISLGLQALTHAISFLRSGASNDEEKARTLIVIIQMASEAARYRYISNRVGVSIRTGTAFQPDPAMLSLENNWDNLSGGVQQSVQDTFPNNVILSSINRQPVVVDSLSHPTVAVLALMLFVCNPPNANQSPLLIRSIVEESKICSSRYEPTVRIGGRDGMCVDVYDDGYHNGNRIIAWKCKDRLEENQLWTLKSDKTIRSNGKCLTTEGYAPGNYVMIYDCTSAVAEATYWEIWDNGTIINPKSALVLSAESSSMGGTLTVQTNEYLMRQGWRTGNNTSPFVTSISGYSDLCMQAQGSNVWLADCDNNKKEQQWALYTDGSIRSVQNTNNCLTSKDHKQGSPIVLMACSNGWASQRWLFKNDGSIYNLHDDMVMDVKRSDPSLKEIILHPYHGKPNQIWLTLF

SEQ ID NO: 138 - protein, 528 aa, Abrus precatorius
  DOMAIN (1)..(251): Abrin-d A chain
  DOMAIN (252)..(261): Linker peptide
  DOMAIN (262)..(528): Abrin-d B chain
QDQVIKFTTEGATSQSYKQFIEALRQRLTGGLIHDIPVLPDPTTVEERNRYITVELSNSERESIEVGIDVTNAYVVAYRAGSQSYFLRDAPASASTYLFPGTQRYSLRFDGSYGDLERWAHQTREEISLGLQALTHAISFLRSGASNDEEKARTLIVIIQMASEAARYRCISNRVGVSIRTGTAFQPDPAMLSLENNWDNLSGGVQQSVQDAFPNNVILSSINRQPVVVDSLSHPTVAVLALMLFVCNPPNANQSPLLIRSIVEESKICSSRYEPTVRIGGRDGMCVDVYDDGYHNGNRIIAWKCKDRLEENQLWTLKSDLTIRSNGKCLTTEGYAPGNYVMIYDCTSAVAEATYWEIWDNGTIINPKSALVLSAESSSMGGTLTVQTNEYLMRQGWRTGNNTSPFVTSISGYSDLCMQAQGSNVWLADCDNNKKEQQWALYTDGSIRSVQNTNNCLTSKDHKQGSPIVLMACSNGWASQRWLFKNDGSIYSLYDDMVMDVKGSDPSLKQIILWPYTGKPNQIWLTLF

SEQ ID NO: 139 - protein, 199 aa, Bordetella pertussis
  DOMAIN (1)..(199): Pertussis toxin subunit 2 (PTX S2)
STPGIVIPPQEQITQHGGPYGRCANKTRALTVAELRGSGDLQEYLRHVTRGWSIFALYDGTYLGGEYGGVIKDGTPGGAFDLKTTFCIMTTRNTGQPATDHYYSNVTATRLLSSTNSRLCAVFVRSGQPVIGACTSPYDGKYWSMYSRLRKMLYLIYVAGISVRVHVSKEEQYYDYEDATFETYALTGISICNPGSSLC

SEQ ID NO: 140 - protein, 199 aa, Bordetella pertussis
  DOMAIN (1)..(199)
  Pertussis toxin subunit 3 (PTX S3)
VAPGIVIPPKALFTQQGGAYGRCPNGTRALTVAELRGNAELQTYLRQITPGWSIYGLYDGTYLGQAYGGIIKDAPPGAGFIYRETFCITTIYKTGQPAADHYYSKVTATRLLASTNSRLCAVFVRDGQSVIGACASPYEGRYRDMYDALRRLLYMIYMSGLAVRVHVSKEEQYYDYEDATFQTYALTGISLCNPAASIC

SEQ ID NO: 141 - protein, 110 aa, Bordetella pertussis [sequence omitted]

SEQ ID NO: 142 - protein, 99 aa, Bordetella pertussis
  DOMAIN (1)..(99): Pertussis toxin subunit 5 (PTX S5)
GLPTHLYKNFTVQELALKLKGKNQEFCLTAFMSGRSLVRACLSDAGHEHDTWFDTMLGFAISAYALKSRIALTVEDSPYPGTPGDLLELQICPLNGYCE

SEQ ID NO: 143 - protein, 141 aa, Escherichia coli
  DOMAIN (1)..(141): C7SSG6 (C7SSG6_ECOLX)
MTIKRFFVCAGVMGCLSLNPAMAEWTGDARDGMFSGVVIDQFHTGQIDNNPYFCIEGKQPGGSSIRACSMKNSSVWGPSFSTLYNQALYFYTTGQLVRIYYEPGVWTYPPFVKALTSNALVGLSTCATSTECFGPDRKKNS

SEQ ID NO: 144 - protein, 141 aa, Escherichia coli
  DOMAIN (1)..(141): Q3ZTX8 (Q3ZTX8_ECOLX)
MTIKRFFVCAGIMGCLSLNPAMAEWTGDARDGMFSGVVITQFHTGQIDNKPYFCIEGKQSAGSSISACSMKNSSVWGASFSTLYNQALYFYTTGQPVRIYYEPGVWTYPPFVKALTSNALVGLSTCTTSTECFGPDRKKNS

SEQ ID NO: 145 - skipped

SEQ ID NO: 146 - protein, 258 aa, Adenia volkensii
  DOMAIN (1)..(258): Volkensin B subunit
DPVCPSGETTAFIVGRDGRCVDVKVEEFFDGNKVQMWPCKSSQNANQLWTLKRDGTIRCQGKCLTVRSPQLYAMIWDCTTFYAPATKWEVWDNGTIINPASGRVLTAPTGEAGVTLNLQFNEYAASQAWRVTNVTVPTVTTIVGYDDLCLETNGNGVWLANCVKGKAQQRWTLYADGTIRSQSTLSKCLACSGSCVKLAKIVNTDCAGSANSRWYFDNYGGIVNLRTGMVMDVKESNPSLNEIIAHPWHGNSNQQWFL

SEQ ID NO: 147 - protein, 263 aa, Viscum album
  DOMAIN (1)..(263): Viscumin B subunit
DDVTCSASEPTVRIVGRNGMCVDVRDDDFHDGNQIQLWPSKSNNDPNQLWTIKRDGTIRSNGSCLTTYGYTAGVYVMIFDCNTAVREATLWEIWGNGTIINPRSNLVLAASSGIKGTTLTVQTLDYTLGQGWLAGNDTAPREVTIYGFRDLCMESNGGSVWVETCVISQQNQRWALYGDGSIRPKQNQDQCLTCGRDSVSTVINIVSCSAGSSGQRWVFTNEGAILNLKNGLAMDVAQANPKLRRIIIYPATGKPNQMWLPVP

SEQ ID NO: 148 - protein, 452 aa, Clostridium tetani
  DOMAIN (1)..(452): Tetanus toxin C-fragment
MKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 149 - protein, 451 aa, Clostridium tetani
  DOMAIN (1)..(451): Tetanus toxin C-fragment
KNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYDTNEYSIISSMKKYSLSIGSGWSVSLKGNNLIWTLKDSAGEVRQITFRDLSDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVAYSSKDVQLKNITDYMYLTNAPSYTNGKLNIYYRRLYSGLKFIIKRYTPNNEIDSFVRSGDFIKLYVSYNNNEHIVGYPKDGNAFNNLDRILRVGYNAPGIPLYKKMEAVKLRDLKTYSVQLKLYDDKDASLGLVGTHNGQIGNDPNRDILIASNWYFNHLKDKTLTCDWYFVPTDEGWTND

SEQ ID NO: 150 - protein, 370 aa, Homo sapiens
  DOMAIN (1)..(370): Proto-oncogene Wnt-1
MGLWALLPGWVSATLLLALAALPAALAANSSGRWWGIVNVASSTNLLTDSKSLQLVLEPSLQLLSRKQRRLIRQNPGILHSVSGGLQSAVRECKWQFRNRRWNCPTAPGPHLFGKIVNRGCRETAFIFAITSAGVTHSVARSCSEGSIESCTCDYRRRGPGGPDWHWGGCSDNIDFGRLFGREFVDSGEKGRDLRFLMNLHNNEAGRTTVFSEMRQECKCHGMSGSCTVRTCWMRLPTLRAVGDVLRDRFDGASRVLYGNRGSNRASRAELLRLEPEDPAHKPPSPHDLVYFEKSPNFCTYSGRLGTAGTAGRACNSSSPALDGCELLCCGRGHRTRTQRVTERCNCTFHWCCHVSCRNCTHTRVLHECL

SEQ ID NO: 151 - protein, 366 aa, Homo sapiens
  DOMAIN (1)..(366): Apolipoprotein A-V
MASMAAVLTWALALLSAFSATQARKGFWDYFSQTSGDKGRVEQIHQQKMAREPATLKDSLEQDLNNMNKFLEKLRPLSGSEAPRLPQDPVGMRRQLQEELEEVKARLQPYMAEAHELVGWNLEGLRQQLKPYTMDLMEQVALRVQELQEQLRVVGEDTKAQLLGGVDEAWALLQGLQSRVVHHTGRFKELFHPYAESLVSGIGRHVQELHRSVAPHAPASPARLSRCVQVLSRKLTLKAKALHARIQQNLDQLREELSRAFAGTGTEEGAGPDPQMLSEEVRQRLQAFRQDTYLQIAAFTRAIDQETEEVQQQLAPPPPGHSAFAPEFQQTDSGKVLSKLQARLDDLWEDITHSLHDQGHSHLGDP

SEQ ID NO: 152 - protein, 770 aa, Homo sapiens
  DOMAIN (1)..(770): Amyloid beta A4 protein
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 153 - protein, 194 aa, Vibrio cholerae
  DOMAIN (1)..(194): Cholera toxin subunit A1
NDDKLYRADSRPPDEIKQSGGLMPRGQSEYFDRGTQMNINLYDHARGTQTGFVRHDDGYVSTSISLRSAHLVGQTILSGHSTYYIYVIATAPNMFNVNDVLGAYSPHPDEQEVSALGGIPYSQIYGWYRVHFGVLDEQLHRNRGYRDRYYSNLDIAPAADGYGLAGFPPEHRAWREEPWIHHAPPGCGNAPRSS

SEQ ID NO: 154 - protein, 6 aa, Artificial Sequence
  Cholera toxin mutant amino acid insertions that renders the mutant Cholera toxin less toxic
APRPGP

SEQ ID NO: 155 - protein, 16 aa, Artificial Sequence
  Cholera toxin mutant amino acid insertions that renders the mutant Cholera toxin less toxic
ASRCAELCCNPACPAP

SEQ ID NO: 156 - protein, 23 aa, Artificial Sequence
  Cholera toxin mutant amino acid insertions that renders the mutant Cholera toxin less toxic
ANSSNYCCELCCNPACTGCYPGP

SEQ ID NO: 157 - protein, 251 aa, Shigella dysenteriae
  DOMAIN (1)..(251): Stx1a A1 (subtype ref)
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVAR

SEQ ID NO: 158 - protein, 251 aa, Enterobacteria phage H19B; identical to SEQ ID NO: 157
  DOMAIN (1)..(251): Stx1b A1 (subtype ref)

SEQ ID NO: 159 - protein, 28 aa, Escherichia coli
  DOMAIN (1)..(28): aa 224-251 of Slt-1A1
ISFGSINAILGSVALILNCHHHASRVAR

SEQ ID NO: 160 - protein, 22 aa, Escherichia coli
  DOMAIN (1)..(22): aa 224-245 of Slt-1A1
ISFGSINAILGSVALILNCHHH

SEQ ID NO: 161 - protein, 17 aa, Escherichia coli
  DOMAIN (1)..(17): aa 224-240 of Slt-1A1
ISFGSINAILGSVALIL

SEQ ID NO: 162 - protein, 315 aa, Shigella dysenteriae [sequence omitted]

SEQ ID NO: 163 - protein, 315 aa, Enterobacteria phage H-19B
  DOMAIN (1)..(315): Stx1b subunit A (subtype ref)
MKIIIFRVLTFFFVIFSVNVVAKEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARMASDEFPSMCPADGRVRGITHNKILWDSSTLGAILMRRTISS

SEQ ID NO: 164 - protein, 315 aa, Escherichia coli [sequence omitted]

SEQ ID NO: 165 - protein, 315 aa, Escherichia coli
  DOMAIN (1)..(315): Stx1c subunit A (ref)
MKIIIFRVLTFFFVIFSVNVVAKEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSVNAILGSVALILNCHHHASRVARIVPNEFPSMCPVDGRVRGITHNKILWDSSTLGAILIRRAISS

SEQ ID NO: 166 - protein, 315 aa, Escherichia coli
  DOMAIN (1)..(315): Stx1d subunit A (subtype ref)
MKIMIFRALTFFFVIFSVNAIAKEFTLDFSTAKKYVDSLNVIRSAIGTPLQTISSGGTSLLMIDSGTGDNLFAVDIMGLEPEEERFNNLRLIVERNNLYVTGFVNRTNNVFYRFADFSHVTFPGTRAVTLSGDSSYTTLQRVAGISRTGMQINRHSLTTSYLDLMSYSGTSLTQSVARAMLRFVTVTAEALRFRQIQRGFRTTLDDLSGRSYVMTAEDVDLTLNWGRLSSILPDYHGQDSVRVGRISFGSINAILGSVALILNCHHHASRVARMTPDEFPSMCPTDGSGRGITHNKILWDSSTLGAILIRRTISS

SEQ ID NO: 167 - protein, 240 aa, Escherichia coli
  DOMAIN (1)..(240): E. coli Heat-labile enterotoxin LT A chain (human strain), A chain without signal peptide
NGDKLYRADSRPPDEIKRSGGLMPRGHNEYFDRGTQMNINLYDHARGTQTGFVRYDDGYVSTSLSLRSAHLAGQSILSGYSTYYIYVIATAPNMFNVNDVLGVYSPHPYEQEVSALGGIPYSQIYGWYRVNFGVIDERLHRNREYRDRYYRNLNIAPAEDGYRLAGFPPDHQAWREEPWIHHAPQGCGNSSRTITGDTCNEETQNLSTIYLRKYQSKVKRQIFSDYQSEVDIYNRIRNEL

SEQ ID NO: 168 - protein, 240 aa, Escherichia coli
  DOMAIN (1)..(240): E. coli Heat-labile enterotoxin LT A chain (porcine strain), A chain without signal peptide
NGDRLYRADSRPPDEIKRSGGLMPRGHNEYFDRGTQMNINLYDHARGTQTGFVRYDDGYVSTSLSLRSAHLAGQSILSGYSTYYIYVIATAPNMFNVNDVLGVYSPHPYEQEVSALGGIPYSQIYGWYRVNFGVIDERLHRNREYRDRYYRNLNIAPAEDGYRLAGFPPDHQAWREEPWIHHAPQGCGNSSRTITGDTCNEETQNLSTIYLREYQSKVKRQIFSDYQSEVDIYNRIRDEL

SEQ ID NO: 169 - protein, 241 aa, Escherichia coli
  DOMAIN (1)..(241): E. coli Heat-labile enterotoxin LT-IIa A chain, A chain without signal peptide
NDFFRADSRTPDEIRRAGGLLPRGQQEAYERGTPININLYEHARGTVTGNTRYNDGYVSTTVTLRQAHLIGQNILGSYNEYYIYVVAPAPNLFDVNGVLGRYSPYPSENEFAALGGIPLSQIIGWYRVSFGAIEGGMQRNRDYRGDLFRGLTVAPNEDGYQLAGFPSNFPAWREMPWSTFAPEQCVPNNKEFKGGVCISATNVLSKYDLMNFKKLLKRRLALTFFMSEDDFIGVHGERDEL

SEQ ID NO: 170 - protein, 31 aa, Escherichia coli
  DOMAIN (1)..(31): aa 178-202 of C166LT-II
YQLAGFPSNFPAWREMPWSTFAPEQCVPNNK

SEQ ID NO: 171 - protein, 243 aa, Escherichia coli
  DOMAIN (1)..(243): E. coli Heat-labile enterotoxin LT-IIb A chain, A chain without signal peptide
NDYFRADSRTPDEVRRSGGLIPRGQDEAYERGTPININLYDHARGTATGNTRYNDGYVSTTTTLRQAHLLGQNMLGGYNEYYIYVVAAAPNLFDVNGVLGRYSPYPSENEYAALGGIPLSQIIGWYRVSFGAIEGGMHRNRDYRRDLFRGLSAAPNEDGYRIAGFPDGFPAWEEVPWREFAPNSCLPNNKASSDTTCASLTNKLSQHDLADFKKYIKRKFTLMTLLSINNDGFFSNNGGKDEL

SEQ ID NO: 172 - protein, 235 aa, Bordetella pertussis
  DOMAIN (1)..(235): Pertussis toxin subunit 1 (= PTX S1)
DDPPATVYRYDSRPPEDVFQNGFTAWGNNDNVLDHLTGRSCQVGSSNSAFVSTSSSRRYTEVYLEHRMQEAVEAERAGRGTGHFIGYIYEVRADNNFYGAASSYFEYVDTYGDNAGRILAGALATYQSEYLAHRRIPPENIRRVTRVYHNGITGETTTTEYSNARYVSQQTRANPNPYTSRRSVASIVGTLVRMAPVIGACMARQAESSEAMAAWSERAGEAMVLVYYESIAYSF

SEQ ID NO: 173 - protein, 347 aa, Escherichia coli
  DOMAIN (1)..(347): Q6EZC2 (Q6EZC2_ECOLX)
MLKILWTYILFLLFISASARAEKPWYFDAIGLTETTMSLTDKNTPVVVSVVDSGVAFIGGLSDSEFAKFSFTQDGSPFPVKKSEALYIHGTAMASLIASRYGIYGVYPHALISSRRVIPDGVQDSWIRAIESIMSNVFLAPGEEKIINISGGQKGVASASVWTELLSRMGRNNDRLIVAAVGNDGADIRKLSAQQRIWPAAYHPVSSVNKKQDPVIRVAALAQYRKGETPVLHGGGITGSRFGNNWVDIAAPGQNITFLRPDAKTGTGSGTSEATAIVSGVLAAMTSCNPRATATELKRTLLESADKYPSLVDKVTEGRVLNAEKAISMFCKKNYIPVRQGRMSEEL

SEQ ID NO: 174 - protein, 347 aa, Escherichia coli
  DOMAIN (1)..(347): Q3ZTX7 (Q3ZTX7_ECOLX)
MLKILWTYILFLLFISASARAEKPWYFDAIGLTETTMSLTDKNTPVVVSVVDSGVAFIGGLSDSEFAKFSFTQDGSPFPVKKSEALYIHGTAMASLIASRYGIYGVYPHALISSRRVIPDGVQDSWIRAIESIMSNVFLAPGEEKIINISGGQKGVASASVWTELLSRMGRNNDRLIVAAVGNDGADIRKLSAQQRIWPAAYHPVSSVNKKQDPVIRVAALAQYRKGETPVLHGGGITGSRFGNNWVDIAAPGQNITFLRPDGKTGTGSGTSEATAIVSGVLAAMTSCNPRATATELKRTLLESADKYPSLVDKVTEGRVLNAEKAISMFCKKNYIPVRQGRMSEEL

SEQ ID NO: 175 - protein, 351 aa, Escherichia coli
  DOMAIN (1)..(351): C7SSG5 (C7SSG5_ECOLX)
MEDIVLKNLRLHILFLLFISVSVRAEKPWYFDAIGLTETTMSLTDKNTPVVVSVVDSGVAFVGGLSDSEFAKFSFTQDGSPFPVKEPEALYIHGTAMASLIASRHEVYGVYPHALISSRRVIPDGVQDSWIRATESIMSNVFLAPGEEKIINISGGQKGISSASVWSELLSRMGRNNERLIVAAVGNDGADIRKLSAQQRIWPAAYHPVSSVNKKQDPVIRVAALAQYRKGETPVLHGGGVTGSRFGNGWVDIAAPGQNITFLKPDGKTGIGSGTSEATAIVSGVLAAMVSCNPRATATELKRTLLESADKYPSLADKVTEGRVLNAEKAISMFCKKNYIPVRQGRMSEEL

SEQ ID NO: 176 - protein, 250 aa, Adenia volkensii
  DOMAIN (1)..(250): Volkensin A subunit
VFPKVPFDVPKATVESYTRFIRVLRDELAGGVSPQGIRRLRNPAEIQPSQGFILIQLTGYVGSVTLIMDVRNAYLLGYLSHNVLYHFNDVSASSIASVFPDAQRRQLPFGGGYPSMRNYAPERDQIDHGIVELAYAVDRLYYSQNNNQIALGLVICAGMVAEASRFRYIEGLVRQSIVGPGDYRTFRPDALMYSIVTQWQTLSERIQGSFNGAFQPVQLGYASDPFYWDNVAQAITRLSLMLFVCSQPPR

SEQ ID NO: 177 - protein, 254 aa, Viscum album
  DOMAIN (1)..(254): Viscumin A subunit
YERLRLRVTHQTTGEEYFRFITLLRDYVSSGSFSNEIPLLRQSTIPVSDAQRFVLVELTNEGGDSITAAIDVTNLYVVAYQAGDQSYFLRDAPRGAETHLFTGTTRSSLPFNGSYPDLERYAGHRDQIPLGIDQLIQSVTALRFPGGSTRTQARSILILIQMISEAARFNPILWRARQYINSGASFLPDVYMLELETSWGQQSTQVQQSTDGVFNNPIRLAIPPGNFVTLTNVRDVIASLAIMLFVCGERPSSS

SEQ ID NO: 178 - protein, 271 aa, Cinnamomum camphora
  DOMAIN (1)..(271): Cinnamomin I A chain
YQTVTFTTKNATKTSYTQFIEALRAQLASGEEPHGIPVMRERSTVPDSKRFILVELSNWAADSPVTLAVDVTNAYVVAYRTGSQSFFLREDNPDPAIENLLPDTKRYTFPFSGSYTDLEGVAGERREEILLGMDPLENAISALWISNLNQQRALARSLIVVIQMVAEAVRFRFIEYRVRGSISRAEMFRPDPAMLSLENKWSALSNAVQQSNQGGVFSSPVELRSISNKPVYVGSVSDRVISGLAIMLFICRSTDRASSDQFIDHMLMIRP

SEQ ID NO: 179 - protein, 271 aa, Cinnamomum camphora
  DOMAIN (1)..(271): Cinnamomin II A chain
YQTVTFTTKNATKTSYTQFIEALRAQLASGEEPHGIPVMRDGSTVPDSKRFILVELSNWAADSPVALAVDVTNAYVVAYRTGSQSFFLREDNPDPAIENLLPDTKRYTFPFSGSYTDLERVAGELREEILLGMDPLENAISALWTSNLNQQRALARSLIVVIQMVAEAVRFRFIEYRVRESITRAEMFRPDPAMLSLENKWSALSNAVQQSNQGGVFSSPVELRSISNKPVYVGSVSDRVISGLAIMLFICRSSDRTSSDQFIDHLLMIRP

SEQ ID NO: 180 - protein, 271 aa, Cinnamomum camphora
  DOMAIN (1)..(271): Cinnamomin III A chain
YQTVTFTTKNATKTSYTQFIEALRAQLASGEEPHGIPVMRERSTVPDSKRFILVELSNWAADSPVTLAVDVTNAYVVAYRTGSQSFFLREDNPDPAIENLLPDTKRYTFPFSGSYTDLERVAGERREEILLGMDPLENAISALWISNLNQQRALARSLIVVIQMVAEAVRFRFIEYRVRESITRAEMFRPDPAMLSLENKWSALSNAVQQSNQGGVFSSPVELRSISNKPVYVGSVSDRVISGLAIMLFICRSTDRASSDQFIDHLLMIRP

SEQ ID NO: 181 - protein, 263 aa, Sambucus nigra
  DOMAIN (1)..(263): Ribosome-inactivating protein SNAI′ A chain
APPTVYPSVSFNLTEANSNEYRHFLQELRGKVILGSHRAFDLPVLNPESKVSDSDRFVLVRLTNPSRKKVTLAIDVVTFYVVAFAQNDRSYFFSGSSEVQRENLFVDTTQEDLNFKGDYTSLEHQVGFGRVYIPLGPKSLAQSISSLSTYKSSAGDNKRLARSLLVVIQMVSEAARFRYIQLRIQASITDAKEFTPDLLMLSMENKWSSMSSEIQQAQPGGAFAQVVKLLDQRNHPIDVTNFRRLFQLTSVAVLLHGCPTVTK

SEQ ID NO: 182 - protein, 273 aa, Sambucus ebulus
  DOMAIN (1)..(273): Ebulin 1 Ribosome-inactivating protein (ebu1) A chain
IDYPSVSFNLAGAKSTTYRDFLKNLRDRVATGTYEVNGLPVLRRESEVQVKNRFVLVRLTNYNGDTVTSAVDVTNLYLVAFSANGNSYFFKDATELQKSNLFLGTTQHTLSFTGNYDNLETAAGTRRESIELGPNPLDGAITSLWYDGGVARSLLVLIQMVPEAARFRYIEQEVRRSLQQLTSFTPNALMLSMENNWSSMSLEVQLSGDNVSPFSGTVQLQNYDHTPRLVDNFEELYKITGIAILLFRCVATKTTHNAIRMPHVLVGEDNKFN

SEQ ID NO: 183 - protein, 280 aa, Sambucus nigra
  DOMAIN (1)..(280): Type 2 ribosome-inactivating protein SNAIf A chain
VTPPVYPSVSFNLTGADTYGPFLRALQEKVILGNHTAFDLPVLNPESQVSDSNRFVLVPLTNPSGDTVTLAIDVVNLYVVAFSSNGRSYFFSGSTAVQRDNLFVDTTQEELNFTGNYISLERQVGFGRVYIPLGPKSLAQAISSLRTYTLSAGDTKPLARGLLVVIQMVSEAARFRYIELRIRTSITDASEFTPDLLMLSMENNWSSMSSEIQQAQPGGIFPGVVQLRDERNNPIEVTNFRRLFELTYIAVLLYGCAPVTSNSYTNNAIDAQIIKMPVFR

SEQ ID NO: 184 - protein, 280 aa, Sambucus nigra
  DOMAIN (1)..(280): Lectin (Q41358 (Q41358_SAMNI)) A chain
VTPPVYPSVSFNLTGADTYEPFLRALQEKVILGNHTAFDLPVLNPESQVSDSNRFVLVPLTNPSGDTVTLAIDVVNLYVVAFSSNGKSYFFSGSTAVQRDNLFVDTTQEELNFTGNYTSLERQVGFGRVYIPLGPKSLDQAISSLRTYTLTAGDTKPLARGLLVVIQMVSEAARFRYIELRIRTSITDASEFTPDLLMLSMENNWSSMSSEIQQAQPGGIFAGVVQLRDERNNSIEVTNFRRLFELTYIAVLLYGCAPVTSSSYSNNAIDAQIIKMPVFR

SEQ ID NO: 185 - protein, 272 aa, Sambucus nigra
  DOMAIN (1)..(272): Ribosome-inactivating protein (AVl) A chain
IDYPSVSFNLDGAKSATYRDFLSNLRKTVATGTYEVNGLPVLRRESEVQVKSRFVLVPLTNYNGNTVTLAVDVTNLYVVAFSGNANSYFFKDATEVQKSNLFVGTKQNTLSFTGNYDNLETAANTRRESIELGPSPLDGAITSLYHGDSVARSLLVVIQMVSEAARFRYIEQEVRRSLQQATSFTPNALMLSMENNWSSMSLEIQQAGNNVSPFFGTVQLLNYDHTHRLVDNFEELYKITGIAILLFRCSSPSNDNAIRMPLDLAGGDNKYN

SEQ ID NO: 186 - protein, 272 aa, Sambucus nigra
  DOMAIN (1)..(272): Type 2 ribosome-inactivating protein Nigrin l A chain
IDYPSVSFNLDGAKSATYRDFLSNLRKTVATGTYEVNGLPVLRRESEVQVKSRFVLVPLTNYNGNTVTLAVDVTNLYVVAFSGNANSYFFKDATEVQKSNLFVGTKQNTLSFTGNYDNLETAANTRRESIELGPSPLDGAITSLYHGDSVARSLLVVIQMVSEAARFRYIEQEVRRSLQQATSFTPNASMLSMENNWSSMSLEIQQAGNNVSPFSGTVQLLNYDHTHRLVDNFEELYKITGIAILLFRCSSPSNDNAIRMPLDLAGEDNKYN

SEQ ID NO: 187 - protein, 272 aa, Sambucus nigra
  DOMAIN (1)..(272): Type 2 ribosome-inactivating protein Nigrin b
IDYPSVSFNLDGAKSATYRDFLSNLRKTVATGTYEVNGLPVLRRESEVQVKSRFVLVPLTNYNGNTVTLAVDVTNLYVVAFSGNANSYFFKDATEVQKSNLFVGTKQNTLSFTGNYDNLETAANTRRESIELGPSPLDGAITSLYHGDSVARSLLVVIQMVSEAARFRYIEQEVRRSLQQATSFTPNALMLSMENNWSSMSLEIQQAGNNVSPFFGTVQLLNYDHTHRLVDNFEELYKITGIAILLFRCSSPSNDNAIRMPLDLAGEDNKYN

SEQ ID NO: 188 - protein, 7 aa, Simian virus 40
  DOMAIN (1)..(7): nuclear localization signal peptide of SV40 Large T-antigen
PKKKRKV

SEQ ID NO: 189 - protein, 16 aa, Unknown
  nuclear localization signal peptide of nucleoplasmin
KRPAATKKAGQAKKKK

SEQ ID NO: 190 - protein, 14 aa, Artificial Sequence
  variable Tet1-flexible linker peptide
  LEGEND (1)..(14): Xaa represents a flexible linker with the sequence GGG, SGSG, or SGSGSG
HLNILSTLWKYRXC

SEQ ID NO: 191 - protein, 679 aa, Homo sapiens
  DOMAIN (1)..(679): serum transferin
VPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 192 - protein, 71 aa, Artificial Sequence
  DRBD peptide with N-terminal Cys
CFFMEELNTYRQKQGVVLKYQELPNSGPPHDRRFTFQVIIDGREFPEGEGRSKKEAKNAAAKLAVEILNKE

SEQ ID NO: 193 - protein, 71 aa, Artificial Sequence
  DRBD peptide with C-terminal Cys
FFMEELNTYRQKQGVVLKYQELPNSGPPHDRRFTFQVIIDGREFPEGEGRSKKEAKNAAAKLAVEILNKEC

SEQ ID NO: 194 - RNA, 21 nt, Artificial Sequence
  GAPDH targeted siRNA - sense strand
  LEGEND (1)..(21): m represents a 2′-O-ME-modified U nucleotide and y represents a 2′-O-ME-modified G nucleotide
ccamcuuccaggagcyagamm

SEQ ID NO: 195 - RNA, 21 nt, Artificial Sequence
  GAPDH targeted siRNA - antisense strand
  LEGEND (1)..(21): m represents a 2′-O-ME-modified U nucleotide and y represents a 2′-O-ME-modified G nucleotide, and wherein the sequence has a 5′-phosphate and deoxy-nucleotides at its 3′ end (dNdN)
ucucgcuccugyaagamggdd

SEQ ID NO: 196 - protein, 116 aa, Unknown
  anti-EGF-R single chain antibody
QVQLVESGGGLVQPGGSLRLPCAASGSIFSLDAWGWYRQAPGKQREMVALVGSDGSTSYADSVKGRFTISRDNANNTFYLQMNSLKPEDTAVYYCYARFQSLYNSWGQGTQVTVSS

SEQ ID NO: 197 - RNA, 21 nt, Artificial Sequence
  fLuc targeted siRNA- sense strand
  LEGEND (1)..(21): m represents a 2′-O-ME-modified U nucleotide and y represents a 2′-O-ME-modified G nucleotide
cuuacycugagmacuucgamm

SEQ ID NO: 198 - RNA, 21 nt, Artificial Sequence
  fLuc targeted siRNA - antisense strand
  LEGEND (1)..(21): Y represents a 2′-O-Me-modified G nucleotide, DD represents two deoxynucleotides at its 3′end (dTdG), sequence has a 5′-phosphate
ucgaaguacucaycguaaydd

SEQ ID NO: 199 - RNA, 21 nt, Artificial Sequence
  non-silencing siRNA control - sense
  LEGEND (1)..(21): m represents a 2′-O-Me-modified U nucleotide
ggamcuuauuucumcggagmm

SEQ ID NO: 200 - RNA, 21 nt, Artificial Sequence
  non-silencing siRNA control - antisense
  LEGEND (1)..(21): m represents a 2′-O-Me-modified U nucleotide, DD represents two deoxynucleotides at its 3′end (dTdT), sequence has a 5′-phosphate
cuccgaagaaamaagamccdd

SEQ ID NO: 201 - DNA, 19 bp, Unknown
  GAPDH siRNA target nucleic acid
ggtcatccatgacaacttt

SEQ ID NO: 202 - DNA, 23 bp, Artificial Sequence
  Gene Racer 5′ primer
cgactggagcacgaggacactga

SEQ ID NO: 203 - DNA, 28 bp, Artificial Sequence
  GAPDH 3′ primer
acgcctgcttcaccaccttcttgatgtc

SEQ ID NO: 204 - DNA, 26 bp, Artificial Sequence
  GAPDH 5′ nested primer
ggacactgacatggactgaaggagta

SEQ ID NO: 205 - DNA, 28 bp, Artificial Sequence
  GAPDH 3′ nested primer
aggccatgccagtgagcttcccgttcag

SEQ ID NO: 206 - RNA, 21 nt, Artificial Sequence
  VEGF targeted siRNA - sense
  LEGEND (1)..(21): DD represents two deoxynucleotides (dTdT)
ggaguacccugaugagaucdd

SEQ ID NO: 207 - RNA, 21 nt, Artificial Sequence
  VEGF targeted siRNA - antisense
  LEGEND (1)..(21): DD represents two deoxynucleotides (dTdT)
gaucucaucaggguacuccdd

SEQ ID NO: 208 - RNA, 21 nt, Artificial Sequence
  Bcl-xL targeted siRNA - sense
  LEGEND (1)..(21): DD represents two deoxynucleotides (dTdT)
gguauuggugagucggaucdd

SEQ ID NO: 209 - RNA, 21 nt, Artificial Sequence
  Bcl-xL targeted siRNA - antisense
  LEGEND (1)..(21): DD represents two deoxynucleotides (dTdT)
gauccgacucaccaauaccdd

SEQ ID NO: 210 - DNA, 21 bp, Artificial Sequence
  KDELR-1 targeted siRNA - sense
cuaccucuauaucaccaaatt

SEQ ID NO: 211 - RNA, 21 nt, Artificial Sequence
  KDELR-1 targeted siRNA - antisense
uuuggugauauagagguagaa

SEQ ID NO: 212 - DNA, 21 bp, Artificial Sequence
  KDELR-2 targeted siRNA - sense
auaggagcaggcaagguagat

SEQ ID NO: 213 - DNA, 21 bp, Artificial Sequence
  KDELR-2 targeted siRNA - antisense
cuaccuugccugcuccuautt

SEQ ID NO: 214 - RNA, 21 nt, Artificial Sequence
  KDELR-3 targeted siRNA - sense
acugauuccagauagauagag

SEQ ID NO: 215 - DNA, 21 bp, Artificial Sequence
  KDELR-3 targeted siRNA - antisense
cuaucuaucuggaaucagutt

SEQ ID NO: 216 - DNA, 21 bp, Artificial Sequence
  Sec61a targeted siRNA - sense
ggaauuugccugcuaaucatt

SEQ ID NO: 217 - RNA, 21 nt, Artificial Sequence
  Sec61a targeted siRNA - antisense
ugauuagcaggcaaauuccag

SEQ ID NO: 218 - DNA, 21 bp, Artificial Sequence
  Derlin-1 targeted siRNA - sense
gcuuagcaauggauaugcatt

SEQ ID NO: 219 - RNA, 21 nt, Artificial Sequence
  Derlin-1 targeted siRNA - antisense
ugcauauccauugcuaagcca

SEQ ID NO: 220 - DNA, 21 bp, Artificial Sequence
  PDIA2 targeted siRNA - sense
gucggaaggugauugaauatt

SEQ ID NO: 221 - DNA, 21 bp, Artificial Sequence
  PDIA2 targeted C239siRNA - antisense
uauucaaucaccuuccgacct

SEQ ID NO: 222 - DNA, 21 bp, Artificial Sequence
  Ero1L targeted siRNA - sense
ggaaugucaucuacgaagatt

SEQ ID NO: 223 - DNA, 21 bp, Artificial Sequence
  Ero1L targeted siRNA - antisense
ucuucguagaugacauuccat

SEQ ID NO: 224 - protein, 110 aa, Artificial Sequence
  module (a)“  + linker
  DOMAIN (8)..(8)
  The lysin at position 8 carries an activatable side chain as branching point
TPQNITDLCAEYHNTQIYTLNDKIFSYTESLAGKREMAIITFKNGAIFQVEVPGSQHIDSQKKAIERMKDTLRIAYLTEAKVEKLCVWNNKTPHAIAAISMANSGSGSGD

SEQ ID NO: 225 - protein, 38 aa, Artificial Sequence
  module (b) + module (c) + linker
  DOMAIN (8)..(8): whereby the epsilon-amino group of the branching Lys residue carries in addition the sequence 12-(aminooxy)dodecanoyl-SGKDSSPSSSPSPK-SGSGSG
CSGSGSGKSGSGSGNASSSRSGLDDINPTVLLKAKDEL

SEQ ID NO: 226 - protein, 30 aa, Artificial Sequence
  Human c-myc tagged IgM-mu peptide
EQKLISEEDLGKPTLYQVSLIMSDTGGTSY

SEQ ID NO: 227 - protein, 70 aa, Escherichia coli
  VARIANT (1)..(70): Stx2d subunit B (subtype variant 3)
ADCAKGKIEFSKYNEDDTFTVKVDGKEYWTSRWNLQPLLQSAQLTGMTVTIKSSTCASGSGFAEVQFNND

SEQ ID NO: 228 - protein, 68 aa, Escherichia coli
  DOMAIN (1)..(68): Stx2e subunit B (subtype ref)
ADCAKGKIEFSKYNEDNTFTVKVSGREYWTNRWNLQPLLQSAQLTGMTVTIISNTCSSGSGFAQVKFN

SEQ ID NO: 229 - protein, 68 aa, Escherichia coli
  DOMAIN (1)..(68): Stx2f subunit B (subtype ref)
ADCAVGKIEFSKYNEDDTFTVKVSGREYWTNRWNLQPLLQSAQLTGMTVTIISNTCSSGSGFAQVKFN

SEQ ID NO: 230 - protein, 68 aa, Escherichia coli
  VARIANT (1)..(68): Stx2f subunit B (subtype variant)
ADCAVGKIEFSKYNEDNTFTVRVSGREYWTNRWNLQPLLQSAQLTGMTVTIISNTCSSGSGFAQVKFN

SEQ ID NO: 231 - protein, 70 aa, Escherichia coli
  DOMAIN (1)..(70): Stx2g subunit B (subtype ref)
ADCAKGKIEFSKYNGDNTFTVKVDGKEYWTNRWNLQPLLQSAQLTGMTVTIKSNTCESGSGFAEVQFNND

SEQ ID NO: 232 - protein, 251 aa, Escherichia coli
  VARIANT (1)..(251): Stx1b A1 (subtype variant)
KEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVAR

SEQ ID NO: 233 - protein, 251 aa, Escherichia coli
  DOMAIN (1)..(251): Stx1c A1 (ref)
KEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSVNAILGSVALILNCHHHASRVAR

SEQ ID NO: 234 - protein, 251 aa, Escherichia coli
  DOMAIN (1)..(251): Stx1d A1 (subtype ref)
KEFTLDFSTAKKYVDSLNVIRSAIGTPLQTISSGGTSLLMIDSGTGDNLFAVDIMGLEPEEERFNNLRLIVERNNLYVTGFVNRTNNVFYRFADFSHVTFPGTRAVTLSGDSSYTTLQRVAGISRTGMQINRHSLTTSYLDLMSYSGTSLTQSVARAMLRFVTVTAEALRFRQIQRGFRTTLDDLSGRSYVMTAEDVDLTLNWGRLSSILPDYHGQDSVRVGRISFGSINAILGSVALILNCHHHASRVAR

SEQ ID NO: 235 - protein, 250 aa, Escherichia coli
  DOMAIN (1)..(250): Stx2a A1 (subtype ref)
REFTIDFSTQQSYVSSLNSIRTEISTPLEHISQGTTSVSVINHTPPGSYFAVDIRGLDVYQARFDHLRLIIEQNNLYVAGFVNTATNTFYRFSDFTHISVPGVTTVSMTTDSSYTTLQRVAALERSGMQISRHSLVSSYLALMEFSGNTMTRDASRAVLRFVTVTAEALRFRQIQREFRQALSETAPVYTMTPGDVDLTLNWGRISNVLPEYRGEDGVRVGRISFNNISAILGTVAVILNCHHQGARSVR

SEQ ID NO: 236 - protein, 250 aa, Escherichia coli
  DOMAIN (1)..(250): Stx2b A1 (ref)
REFTIDFSTQQSYVSSLNSIRTEISTPLEHISQGTTSVSVINHTPPGSYFAVDIRGLDVYQARFDHLRLIIERNNLYVAGFVNTATNTSYRFSDFAHISVPGVTTVSMTTDSSYTTLQRVAALERSGMQISRHSLVSSYLALMEFSGNAMTRDASRAVLRFVTVTAEALRFRQIQREFRLALSETAPVYTMTPEEVELTLNWGRISNVLPEFRGEGGVKMGRISFNNISAILGTVAVILNCHHQGARSVR

SEQ ID NO: 237 - protein, 250 aa, Escherichia coli; identical to SEQ ID NO: 235
  DOMAIN (1)..(250): Stx2c A1 (subtype ref)

SEQ ID NO: 238 - protein, 250 aa, Escherichia coli; identical to SEQ ID NO: 235
  VARIANT (1)..(250): Stx2c A1 (subtype variant 1)

SEQ ID NO: 239 - protein, 250 aa, Escherichia coli
  VARIANT (1)..(250): Stx2c A1 (subtype variant 2)
REFTIDFSTQQSYVSSLNTIRTEISTPLEHISQGTTSVSVINHTPPGSYFAVDIRGLDVYQARFDHLRLIIEQNNLYVAGFVNTATNTFYRFSDFTHISVPGVTTVSMTTDSSYTTLQRVAALERSGMQISRHSLVSSYLALMEFSGNTMTRDASRAVLRFVTVTAEALRFRQIQREFRQALSETAPVYTMTPGDVDLTLNWGRISNVLPEYRGEDGVRVGRISFNNISAILGTVAVILNCHHQGARSVR

SEQ ID NO: 240 - protein, 250 aa, Escherichia coli; identical to SEQ ID NO: 235
  DOMAIN (1)..(250): Stx2d A1 (subtype ref)

SEQ ID NO: 241 - protein, 250 aa, Escherichia coli; identical to SEQ ID NO: 235
  VARIANT (1)..(250): Stx2d A1 (subtype variant 1)

SEQ ID NO: 242 - protein, 250 aa, Escherichia coli
  VARIANT (1)..(250): Stx2d A1 (subtype variant 2)
REFTIDFSTQQSYVSSLNSIRTEISTPLEHISQGTTSVSVINHTPPGSYFAVDIRGLDVYQARFDHLRLIIEQNNLYVAGFVNTATNTFYRFSDFAHISVPGVTTVSMTTDSSYTTLQRVAALERSGMQISRHSLVSSYLALMEFSGNTMTRDASRAVLRFVTVTAEALRFRQIQREFRQALSETAPVYTMTPGDVDLTLNWGRISNVLPEYRGEDGVRVGRISFNNISAILGTVAVILNCHHQGARSVR

SEQ ID NO: 243 - protein, 250 aa, Escherichia coli; identical to SEQ ID NO: 239
  VARIANT (1)..(250): Stx2d A1 (subtype variant 3)

SEQ ID NO: 244 - protein, 250 aa, Escherichia coli
  VARIANT (1)..(250): Stx2d A1 (subtype variant 4)
REFTIDFSTQQSYVSSLNSIRTEISTPLEHISQGTTSVSVINHTPPGSYFAVDIRGLDVYQARFDHLRLIIEQNNLYVAGFVNTATNTFYRFSDFTHISVPGVTTVSMTTDSSYTTLQRVAALERSGMQISRHSLVSSYLALMEFSGNTMTRDASRAVLRFVTVTAEALRFRQIQREFRQVLSETAPVYTMTPGDVDLTLNWGRISNVLPEYRGEDGVRVGRISFNNISAILGTVAVILNCHHQGARSVR

SEQ ID NO: 245 - protein, 250 aa, Escherichia coli
  VARIANT (1)..(250): Stx2d A1 (subtype variant 5)
REFTIDFSTQQSYVSSLNSIRAEISTPLEHISQGTTSVSVINHTPPGSYFAVDIRGLDVYQARFDHLRLIIEQNNLYVAGFVNTATNTFYRFSDFTHISVPGVTTVSMTTDSSYTTLQRVAALERSGMQISRHSLVSSYLALMEFSGNTMTRDASRAVLRFVTVTAEALRFRQIQREFRQALSETAPVYTMMPGDVDLTLNWGRISNVLPEYRGEDGVRVGRISFNNISAILGTVAVILNCHHQGARSVR

SEQ ID NO: 246 - protein, 250 aa, Escherichia coli
  DOMAIN (1)..(250): Stx2e A1 (subtype ref)
QEFTIDFSTQQSYVSSLNSIRTAISTPLEHISQGATSVSVINHTPPGSYISVGIRGLDVYQERFDHLRLIIERNNLYVAGFVNTTTNTFYRFSDFAHISLPGVTTISMTTDSSYTTLQRVAALERSGMQISRHSLVSSYLALMEFSGNTMTRDASRAVLRFVTVTAEALRFRQIQREFRLALSETAPVYTMTPEDVDLTLNWGRISNVLPEYRGEAGVRVGRISFNNISAILGTVAVILNCHHQGARSVR

SEQ ID NO: 247 - protein, 250 aa, Escherichia coli; identical to SEQ ID NO: 246
  VARIANT (1)..(250): Stx2e A1 (subtype variant 1)

SEQ ID NO: 248 - protein, 250 aa, Escherichia coli; identical to SEQ ID NO: 246
  VARIANT (1)..(250): Stx2e A1 (subtype variant 2)

SEQ ID NO: 249 - protein, 250 aa, Escherichia coli
  VARIANT (1)..(250): Stx2e A1 (subtype variant 3)
REFTIDFSTQQSYVSSLNSIRTAISTPLEHISQGATSVSVINHTPPGSYISVGIRGLDVYQERFDHLRLIIERNNLYVAGFVNTTTNTFYRFSDFAHISLPGVTTISMTTDSSYTTLQRVAALERSGMQISRHSLVSSYLALMEFSGNTMTRDASRAVLRFVTVTAEALRFRQIQREFRLALSETAPVYTMTPEDVDLTLNWGRISNVLPEYRGEAGVRVGRISFNNISAILGTVAVILNCHHQGARSVR

SEQ ID NO: 250 - protein, 250 aa, Escherichia coli
  VARIANT (1)..(250): Stx2e A1 (subtype variant 4)
QEFTIDFSTQQSYVSSLNSIRTAISTPLEHISQGATSVSVINHTPLGSYISVGIRGLDVYQERFDHLRLIIERNNLYVAGFVNTTTNTFYRFSDFAHISLPGVTTISMTTDSSYTTLQRVAALERSGMQISRHSLVSSYLALMEFSGNTMTRDASRAVLRFVTVTAEALRFRQIQREFRLALSETAPVYTMTPEDVDLTLNWGRISNVLPEYRGEAGVRVGRISFNNISAILGTVAVILNCHHQGARSVR

SEQ ID NO: 251 - protein, 250 aa, Escherichia coli
  VARIANT (1)..(250): Stx2e A1 (subtype variant 5)
QEFTIDFSTQQSYVSSLNSIRTAISTPLEHISQGATSVSVINHTPPGSYISVGIRGLDVYQERFDHLRLIIERNNLYVAGFVNTTTNTFYRFSDFAHISLPGVTTISMTTDSSYTTLQRVAALERSGMQISRHSLVSSYLALMEFSGNTMTRDASRAVLRFVTVTAEALRFRQIQREFRLALSETAPVYTMTPEDVDLTLNWGRISNVLPEYRGEAGVRVGRIFFNNISAILGTVAVILNCHHQGARSVR

SEQ ID NO: 252 - protein, 250 aa, Escherichia coli
  DOMAIN (1)..(250): Stx2f A1 (subtype ref)
DEFTVDFSSQKSYVDSLNSIRSAISTPLGNISQGGVSVSVINHVPGGNYISLNVRGLDPYSERFNHLRLIMERNNLYVAGFINTETNTFYRFSDFSHISVPDVITVSMTTDSSYSSLQRIADLERTGMQIGRHSLVGSYLDLMEFRGRSMTRASSRAMLRFVTVIAEALRFRQIQRGFRPALSEASPLYTMTAQDVDLTLNWGRISNVLPEYRGEEGVRIGRISFNSLSAILGSVAVILNCHSTGSYSVR

SEQ ID NO: 253 - protein, 250 aa, Escherichia coli
  VARIANT (1)..(250): Stx2f A1 (subtype variant)
DEFTVDFSSQKSYVDSLNSIRSAISTPLGNISQGGISVSVINHVPGGNYISLNVRGLEPYSERFNHLRLIMERNNLYVAGFINTETNTFYRFSDFSHISVPDVITVSMTTDSSYSSLQRIADLERTGMQIGRHSLVGSYLDLMEFRGRSMTRASSRAMLRFVTVIAEALRFRQIQRGFRPALSEASPLYTMTAQDVDLTLNWGRISNVLPEYRGEEGVRIGRISFNSLSAILGSVAVILNCHSTGSYSVR

SEQ ID NO: 254 - protein, 250 aa, Escherichia coli
  DOMAIN (1)..(250): Stx2g A1 (subtype ref assigned arbitrarily)
REFTIDFSTQQTYVSSLNSIRTEISTPLEHISQGATSVSVINHTPPGSYISVDIRGLDVYQARFDHLRLIIEQNNLYVAGFVNTATNTFYRFSDFTHISVPGVTTVSMTTDSSYTTLQRVAALERSGMQISRHSLVSSYLALMEFSGNTMTRDASRAVLRFVTVTAEALRFRQIQREFRLALSETAPVYTMTPEDVDLTLNWGRISNVLPEYRGEDGVRVGRISFNNISAILGTVAVILNCHHQGARSVR

SEQ ID NO: 255 - protein, 250 aa, Escherichia coli
  VARIANT (1)..(250): Stx2g A1 (subtype variant 1)
REFTIDFSTQQSYVSSLNSIRTEISTSLEHISQGATSVSVINHTPPGSYISVDIRGLDVYQARFDHLRLIIEQNNLYVAGFVNTATNTFYRFSDFTHISVPGVTTVSMTTDSSYTTQQRVAALERSGMQISRHSLVSSYLALMEFSGNTMTRDASRAVLRFVTVTAEALRFRQIQREFRLALSETAPVYTMTPEDVDLTLNWGRISNVLPEYRGEDSVRVGRISFNNISAILGTVAVILNCHHQGTRSVR

SEQ ID NO: 256 - protein, 250 aa, Escherichia coli; identical to SEQ ID NO: 235
  VARIANT (1)..(250): Stx2 A1 (sequence variant)

SEQ ID NO: 257 - protein, 319 aa, Escherichia coli
  DOMAIN (1)..(319): Stx2a subunit A (subtype ref)
MKCILFKWVLCLLLGFSSVSYSREFTIDFSTQQSYVSSLNSIRTEISTPLEHISQGTTSVSVINHTPPGSYFAVDIRGLDVYQARFDHLRLIIEQNNLYVAGFVNTATNTFYRFSDFTHISVPGVTTVSMTTDSSYTTLQRVAALERSGMQISRHSLVSSYLALMEFSGNTMTRDASRAVLRFVTVTAEALRFRQIQREFRQALSETAPVYTMTPGDVDLTLNWGRISNVLPEYRGEDGVRVGRISFNNISAILGTVAVILNCHHQGARSVRAVNEESQPECQITGDRPVIKINNTLWESNTAAAFLNRKSQFLYTTGK

SEQ ID NO: 258 - protein, 319 aa, Escherichia coli
  DOMAIN (1)..(319): Stx2b subunit A (ref)
MKCILLKWVLCLLLGFSSVSYSREFTIDFSTQQSYVSSLNSIRTEISTPLEHISQGTTSVSVINHTPPGSYFAVDIRGLDVYQARFDHLRLIIERNNLYVAGFVNTATNTSYRFSDFAHISVPGVTTVSMTTDSSYTTLQRVAALERSGMQISRHSLVSSYLALMEFSGNAMTRDASRAVLRFVTVTAEALRFRQIQREFRLALSETAPVYTMTPEEVELTLNWGRISNVLPEFRGEGGVKMGRISFNNISAILGTVAVILNCHHQGARSVRAVNEEIQPECQITGDRPVIRINNTLWESNTAAAFLNRRAHSLNTSGE

SEQ ID NO: 259 - protein, 319 aa, Escherichia coli
  DOMAIN (1)..(319): Stx2c subunit A (subtype ref)
MKCILFKWVLCLLLGFSSVSYSREFTIDFSTQQSYVSSLNSIRTEISTPLEHISQGTTSVSVINHTPPGSYFAVDIRGLDVYQARFDHLRLIIEQNNLYVAGFVNTATNTFYRFSDFTHISVPGVTTVSMTTDSSYTTLQRVAALERSGMQISRHSLVSSYLALMEFSGNTMTRDASRAVLRFVTVTAEALRFRQIQREFRQALSETAPVYTMTPGDVDLTLNWGRISNVLPEYRGEDGVRVGRISFNNISAILGTVAVILNCHHQGARSVRAVNEDSQPECQITGDRPVIKINNTLWESNTAAAFLNRKSQFLYTTGK

SEQ ID NO: 260 - protein, 319 aa, Escherichia coli; identical to SEQ ID NO: 257
  VARIANT (1)..(319): Stx2c subunit A (subtype variant 1)

SEQ ID NO: 261 - protein, 319 aa, Escherichia coli
  VARIANT (1)..(319): Stx2c subunit A (subtype variant 2)
MKCILFKWVLCLLLGFSSVSYSREFTIDFSTQQSYVSSLNTIRTEISTPLEHISQGTTSVSVINHTPPGSYFAVDIRGLDVYQARFDHLRLIIEQNNLYVAGFVNTATNTFYRFSDFTHISVPGVTTVSMTTDSSYTTLQRVAALERSGMQISRHSLVSSYLALMEFSGNTMTRDASRAVLRFVTVTAEALRFRQIQREFRQALSETAPVYTMTPGDVDLTLNWGRISNVLPEYRGEDGVRVGRISFNNISAILGTVAVILNCHHQGARSVRAVNEDSQPECQITGDRPVIKINNTLWESNTAAAFLNRKSQFLYTTGK

SEQ ID NO: 262 - protein, 319 aa, Escherichia coli
  DOMAIN (1)..(319): Stx2d subunit A (subtype ref)
MKCILFKWVLCLLLGFSSVSYSREFTIDFSTQQSYVSSLNSIRTEISTPLEHISQGTTSVSVINHTPPGSYFAVDIRGLDVYQARFDHLRLIIEQNNLYVAGFVNTATNTFYRFSDFTHISVPGVTTVSMTTDSSYTTLQRVAALERSGMQISRHSLVSSYLALMEFSGNTMTRDASRAVLRFVTVTAEALRFRQIQREFRQALSETAPVYTMTPGDVDLTLNWGRISNVLPEYRGEDGVRVGRISFNNISAILGTVAVILNCHHQGARSVRAVNEESQPECQITGDRPVIKINNTLWESNTAAAFLNRKSQSLYTTGE

SEQ ID NO: 263 - protein, 319 aa, Escherichia coli
  VARIANT (1)..(319)
  Stx2d subunit A (subtype variant 1)
MKCILLKWVLCLLLGFSSVSYSREFTIDFSTQQSYVSSLNSIRTEISTPLEHISQGTTSVSVINHTPPGSYFAVDIRGLDVYQARFDHLRLIIEQNNLYVAGFVNTATNTFYRFSDFTHISVPGVTTVSMTTDSSYTTLQRVAALERSGMQISRHSLVSSYLALMEFSGNTMTRDASRAVLRFVTVTAEALRFRQIQREFRQALSETAPVYTMTPGDVDLTLNWGRISNVLPEYRGEDGVRVGRISFNNISAILGTVAVILNCHHQGARSVRAVNEESQPECQITGDRPVIKINNTLWESNTAAAFLNRKSQSLYTTGE

SEQ ID NO: 264 - protein, 319 aa, Escherichia coli
  VARIANT (1)..(319): Stx2d subunit A (subtype variant 2)
MKCILFKWVLCLLLGFSSVSYSREFTIDFSTQQSYVSSLNSIRTEISTPLEHISQGTTSVSVINHTPPGSYFAVDIRGLDVYQARFDHLRLIIEQNNLYVAGFVNTATNTFYRFSDFAHISVPGVTTVSMTTDSSYTTLQRVAALERSGMQISRHSLVSSYLALMEFSGNTMTRDASRAVLRFVTVTAEALRFRQIQREFRQALSETAPVYTMTPGDVDLTLNWGRISNVLPEYRGEDGVRVGRISFNNISAILGTVAVILNCHHQGARSVRAVNEESQPECQITGDRPVIKINNTLWESNTAAAFLNRKSQSLYTTGE

SEQ ID NO: 265 - protein, 319 aa, Escherichia coli
  VARIANT (1)..(319): Stx2d subunit A (subtype variant 3)
MKCILFKWVLCLLLGFSSVSYSREFTIDFSTQQSYVSSLNTIRTEISTPLEHISQGTTSVSVINHTPPGSYFAVDIRGLDVYQARFDHLRLIIEQNNLYVAGFVNTATNTFYRFSDFTHISVPGVTTVSMTTDSSYTTLQRVAALERSGMQISRHSLVSSYLALMEFSGNTMTRDASRAVLRFVTVTAEALRFRQIQREFRQALSETAPVYTMTPGDVDLTLNWGRISNVLPEYRGEDGVRVGRISFNNISAILGTVAVILNCHHQGARSVRAVNEESQPECQITGDRPVIKINNTLWESNTAAAFLNRKSQSLYTTGE

SEQ ID NO: 266 - protein, 319 aa, Escherichia coli
  VARIANT (1)..(319): Stx2d subunit A (subtype variant 4)
MKCILFKWVLCLLLGFSSVSYSREFTIDFSTQQSYVSSLNSIRTEISTPLEHISQGTTSVSVINHTPPGSYFAVDIRGLDVYQARFDHLRLIIEQNNLYVAGFVNTATNTFYRFSDFTHISVPGVTTVSMTTDSSYTTLQRVAALERSGMQISRHSLVSSYLALMEFSGNTMTRDASRAVLRFVTVTAEALRFRQIQREFRQVLSETAPVYTMTPGDVDLTLNWGRISNVLPEYRGEDGVRVGRISFNNISAILGTVAVILNCHHQGARSVRAVNEDSQPECQITGDRPVIKINNTLWESNTAAAFLNRKSQSLYTTGE

SEQ ID NO: 267 - protein, 319 aa, Escherichia coli
  VARIANT (1)..(319): Stx2d subunit A (subtype variant 5)
MKCILFKWVLCLLLGFSSVSYSREFTIDFSTQQSYVSSLNSIRAEISTPLEHISQGTTSVSVINHTPPGSYFAVDIRGLDVYQARFDHLRLIIEQNNLYVAGFVNTATNTFYRFSDFTHISVPGVTTVSMTTDSSYTTLQRVAALERSGMQISRHSLVSSYLALMEFSGNTMTRDASRAVLRFVTVTAEALRFRQIQREFRQALSETAPVYTMMPGDVDLTLNWGRISNVLPEYRGEDGVRVGRISFNNISAILGTVAVILNCHHQGARSVRAVNEESQPECQITGDRPVIKINNTLWESNTAAAFLNRKSQSLYTTGE

SEQ ID NO: 268 - protein, 319 aa, Escherichia coli
  DOMAIN (1)..(319): Stx2e subunit A (subtype ref)
MKCILLKWILCLLLGFSSVSYSQEFTIDFSTQQSYVSSLNSIRTAISTPLEHISQGATSVSVINHTPPGSYISVGIRGLDVYQERFDHLRLIIERNNLYVAGFVNTTTNTFYRFSDFAHISLPGVTTISMTTDSSYTTLQRVAALERSGMQISRHSLVSSYLALMEFSGNTMTRDASRAVLRFVTVTAEALRFRQIQREFRLALSETAPVYTMTPEDVDLTLNWGRISNVLPEYRGEAGVRVGRISFNNISAILGTVAVILNCHHQGARSVRAVNEESQPECQITGDRPVIKINNTLWESNTAAAFLNRKSQPLYTTGE

SEQ ID NO: 269 - protein, 319 aa, Escherichia coli
  DOMAIN (1)..(319): Stx2e subunit A (subtype variant 1)
MKCILLKWILCLLLGFSSVSYSQEFTIDFSTQQSYVSSLNSIRTAISTPLEHISQGATSVSVINHTPPGSYISVGIRGLDVYQERFDHLRLIIERNNLYVAGFVNTTTNTFYRFSDFAHISLPGVTTISMTTDSSYTTLQRVAALERSGMQISRHSLVSSYLALMEFSGNTMTRDASRAVLRFVTVTAEALRFRQIQREFRLALSETAPVYTMTPEDVDLTLNWGRISNVLPEYRGEAGVRVGRISFNNISAILGTVAVILNCHHQGARSVRAVNEESQPECQITGDRPVIKINNKLWESNTAAAFLNRKSQPLYTTGE

SEQ ID NO: 270 - protein, 319 aa, Escherichia coli
  VARIANT (1)..(319): Stx2e subunit A (subtype variant 2)
MKCILLKWILCLLLGFSSVSYSQEFTIDFSTQQSYVSSLNSIRTAISTPLEHISQGATSVSVINHTPPGSYISVGIRGLDVYQERFDHLRLIIERNNLYVAGFVNTTTNTFYRFSDFAHISLPGVTTISMTTDSSYTTLQRVAALERSGMQISRHSLVSSYLALMEFSGNTMTRDASRAVLRFVTVTAEALRFRQIQREFRLALSETAPVYTMTPEDVDLTLNWGRISNVLPEYRGEAGVRVGRISFNNISAILGTVAVILNCHHQGARSVRAVNEESQPECQITGDRPVIKINNTLWESNTAAAFLNRKSQSLYTTGE

SEQ ID NO: 271 - protein, 319 aa, Escherichia coli
  VARIANT (1)..(319): Stx2e subunit A (subtype variant 3)
MKCILLKWILCLLLGFSSVSYSREFTIDFSTQQSYVSSLNSIRTAISTPLEHISQGATSVSVINHTPPGSYISVGIRGLDVYQERFDHLRLIIERNNLYVAGFVNTTTNTFYRFSDFAHISLPGVTTISMTTDSSYTTLQRVAALERSGMQISRHSLVSSYLALMEFSGNTMTRDASRAVLRFVTVTAEALRFRQIQREFRLALSETAPVYTMTPEDVDLTLNWGRISNVLPEYRGEAGVRVGRISFNNISAILGTVAVILNCHHQGARSVRAVNEESQPECQITGDRPVIKINNTLWESNTAAAFLNRKSQSLYTTGE

SEQ ID NO: 272 - protein, 319 aa, Escherichia coli
  VARIANT (1)..(319): Stx2e subunit A (subtype variant 4)
MKCILLKWILCLLLGFSSVSYSQEFTIDFSTQQSYVSSLNSIRTAISTPLEHISQGATSVSVINHTPLGSYISVGIRGLDVYQERFDHLRLIIERNNLYVAGFVNTTTNTFYRFSDFAHISLPGVTTISMTTDSSYTTLQRVAALERSGMQISRHSLVSSYLALMEFSGNTMTRDASRAVLRFVTVTAEALRFRQIQREFRLALSETAPVYTMTPEDVDLTLNWGRISNVLPEYRGEAGVRVGRISFNNISAILGTVAVILNCHHQGARSVRAVNEESQPECQITGDRPVIKINNTLWESNTAAAFLNRKSQPLYTTGE

SEQ ID NO: 273 - protein, 319 aa, Escherichia coli
  VARIANT (1)..(319): Stx2e subunit A (subtype variant 5)
MKCILLKWILCLLLGFSSVSYSQEFTIDFSTQQSYVSSLNSIRTAISTPLEHISQGATSVSVINHTPPGSYISVGIRGLDVYQERFDHLRLIIERNNLYVAGFVNTTTNTFYRFSDFAHISLPGVTTISMTTDSSYTTLQRVAALERSGMQISRHSLVSSYLALMEFSGNTMTRDASRAVLRFVTVTAEALRFRQIQREFRLALSETAPVYTMTPEDVDLTLNWGRISNVLPEYRGEAGVRVGRIFFNNISAILGTVAVILNCHHQGARSVRAVNEESQPECQITGDRPVIKINNTLWESNTAAAFLNRKSQPLYTTGE

SEQ ID NO: 274 - protein, 319 aa, Escherichia coli
  DOMAIN (1)..(319): Stx2f subunit A (subtype ref)
MRHILLKLVLFFCVCLSSASYADEFTVDFSSQKSYVDSLNSIRSAISTPLGNISQGGVSVSVINHVPGGNYISLNVRGLDPYSERFNHLRLIMERNNLYVAGFINTETNTFYRFSDFSHISVPDVITVSMTTDSSYSSLQRIADLERTGMQIGRHSLVGSYLDLMEFRGRSMTRASSRAMLRFVTVIAEALRFRQIQRGFRPALSEASPLYTMTAQDVDLTLNWGRISNVLPEYRGEEGVRIGRISFNSLSAILGSVAVILNCHSTGSYSVRSVSQKQKTECQIVGDRAAIKVNNVLWEANTIAALLNRKPQDLTEPNQ

SEQ ID NO: 275 - protein, 319 aa, Escherichia coli
  VARIANT (1)..(319): Stx2f subunit A (subtype variant 1)
MRHILLKLVLFFCVCLSSVSYADEFTVDFSSQKSYVDSLNSIRSAISTPLGNISQGGISVSVINHVPGGNYISLNVRGLEPYSERFNHLRLIMERNNLYVAGFINTETNTFYRFSDFSHISVPDVITVSMTTDSSYSSLQRIADLERTGMQIGRHSLVGSYLDLMEFRGRSMTRASSRAMLRFVTVIAEALRFRQIQRGFRPALSEASPLYTMTAQDVDLTLNWGRISNVLPEYRGEEGVRIGRISFNSLSAILGSVAVILNCHSTGSYSVRSVSQKQKTECQIVGDRAAIKVNNVLWEANTIAALLNRKPQGLTEPNQ

SEQ ID NO: 276 - protein, 319 aa, Escherichia coli
  VARIANT (1)..(319): Stx2f subunit A (subtype variant 2)
MRYILLKLVLFFCVCLSSASYADEFTVDFSSQKSYVDSLNSIRSAISTPLGNISQGGVSVSVINHVPGGNYISLNVRGLDPYSERFNHLRLIMERNNLYVAGFINTETNTFYRFSDFSHISVPDVITVSMTTDSSYSSLQRIADLERTGMQIGRHSLVGSYLDLMEFRGRSMTRASSRAMLRFVTVIAEALRFRQIQRGFRPALSEASPLYTMTAQDVDLTLNWGRISNVLPEYRGEEGVRIGRISFNSLSAILGSVAVILNCHSTGSYSVRSVSQKQKTECQIVGDRAAIKVNNVLWEANTIAALLNRKPQDLTEPNQ

SEQ ID NO: 277 - protein, 319 aa, Escherichia coli
  DOMAIN (1)..(319): Stx2g subunit A (subtype ref assigned arbitrarily)
MKCILFKWVLCLLLGFSSVSYSREFTIDFSTQQTYVSSLNSIRTEISTPLEHISQGATSVSVINHTPPGSYISVDIRGLDVYQARFDHLRLIIEQNNLYVAGFVNTATNTFYRFSDFTHISVPGVTTVSMTTDSSYTTLQRVAALERSGMQISRHSLVSSYLALMEFSGNTMTRDASRAVLRFVTVTAEALRFRQIQREFRLALSETAPVYTMTPEDVDLTLNWGRISNVLPEYRGEDGVRVGRISFNNISAILGTVAVILNCHHQGARSVRYVNEEMQPKCQISGDRPVIKINNTLWESNTAAAFLNRKSQSLYTTGE

SEQ ID NO: 278 - protein, 319 aa, Escherichia coli
  VARIANT (1)..(319): Stx2g subunit A (subtype variant 1)
MKCILFKWVLCLLLGFSSVSYSREFTIDFSTQQSYVSSLNSIRTEISTSLEHISQGATSVSVINHTPPGSYISVDIRGLDVYQARFDHLRLIIEQNNLYVAGFVNTATNTFYRFSDFTHISVPGVTTVSMTTDSSYTTQQRVAALERSGMQISRHSLVSSYLALMEFSGNTMTRDASRAVLRFVTVTAEALRFRQIQREFRLALSETAPVYTMTPEDVDLTLNWGRISNVLPEYRGEDSVRVGRISFNNISAILGTVAVILNCHHQGTRSVRYVNEEMQPECQISGDRPVIKINNTLWESNTAAAFLNRKSQSLYTTGE

SEQ ID NO: 279 - protein, 319 aa, Escherichia coli; identical to SEQ ID NO: 262
  VARIANT (1)..(319): Stx2 subunit A (sequence variant)

SEQ ID NO: 280 - protein, 40 aa, Rattus norvegicus
  DOMAIN (1)..(40): AChE peptide
DTLDEAERQWKAEFHRWSSYMVHWKNQFDHYSKQERCSDL

SEQ ID NO: 281 - protein, 40 aa, Rattus norvegicus
  DOMAIN (1)..(40): AChE peptide
DTLDEAERQWKAEFHRWSSYMVHWKNQFDHYSKQERSSDL

SEQ ID NO: 282 - protein, 40 aa, Torpedo californica
  DOMAIN (1)..(40): AChE peptide
ETIDEAERQWKTEFHRWSSYMMHWKNQFDQYSRHENCAEL

SEQ ID NO: 283 - protein, 40 aa, Torpedo californica
  DOMAIN (1)..(40): AChE peptide
ETIDEAERQWKTEFHRWSSYMMHWKNQFDQYSRHENSAEL

SEQ ID NO: 284 - protein, 40 aa, Torpedo californica
  DOMAIN (1)..(40): AChE peptide
ETIDEAERQWKTEFHRWSCYMMHWKNQFDQYSRHENCAEL

SEQ ID NO: 285 - protein, 40 aa, Torpedo californica
  DOMAIN (1)..(40): AChE peptide
ETIDEAERQWKTEFHRWSCYMMHWKNQFDQYSRHENSAEL

SEQ ID NO: 286 - protein, 40 aa, Torpedo californica
  DOMAIN (1)..(40): AChE peptide
ETIDEAERQWKTEFHRWSSYCMHWKNQFDQYSRHENCAEL

SEQ ID NO: 287 - protein, 40 aa, Torpedo californica
  DOMAIN (1)..(40): AChE peptide
ETIDEAERQWKTEFHRWSSYCMHWKNQFDQYSRHENSAEL

SEQ ID NO: 288 - protein, 40 aa, Torpedo californica
  DOMAIN (1)..(40): AChE peptide
ETIDEAERQWKTEFHRWSCYCMHWKNQFDQYSRHENCAEL

SEQ ID NO: 289 - protein, 40 aa, Torpedo californica
  DOMAIN (1)..(40): AChE peptide
ETIDEAERQWKTEFHRWSCYCMHWKNQFDQYSRHENSAEL

SEQ ID NO: 290 - protein, 40 aa, Rattus norvegicus
  DOMAIN (1)..(40): AChE peptide
  DOMAIN (37)..(37): Xaa is Cys or Ser
DTLDEAERQWRAEFHRWSSYMVHWKNQFDHYSKQERXSDL

SEQ ID NO: 291 - protein, 40 aa, Torpedo californica
  DOMAIN (1)..(40): AChE peptide
  DOMAIN (19)..(19): Xaa is Cys or Ser
  DOMAIN (21)..(21): Xaa is Cys or Met
  DOMAIN (27)..(27): Xaa is Cys or Ser
ETIDEAERQWKTEFHRWSXYXMHWKNQFDQYSRHENXAEL

SEQ ID NO: 292 - protein, 40 aa, Artificial Sequence
  Mutated AChE peptide derived from Rattus norvegicus
  DOMAIN (1)..(40): Mutated AChE peptide
DTLDEAERQWRAEFHRWSSYMVHWKNQFDHYSKQERKDEL

SEQ ID NO: 293 - protein, 40 aa, Artificial Sequence
  Mutated AChE peptide derived from Torpedo californica
  DOMAIN (1)..(40): Mutated AChE peptide
ETIDEAERQWKTEFHRWSSYMMHWKNQFDQYSRHENKDEL

SEQ ID NO: 294 - protein, 40 aa, Artificial Sequence
  Mutated AChE peptide derived from Torpedo californica
  DOMAIN (1)..(40): Mutated AChE peptide
ETIDEAERQWKTEFHRWSCYMMHWKNQFDQYSRHENKDEL

SEQ ID NO: 295 - protein, 40 aa, Artificial Sequence
  Mutated AChE peptide derived from Torpedo californica
  DOMAIN (1)..(40): Mutated AChE peptide
ETIDEAERQWKTEFHRWSSYCMHWKNQFDQYSRHENKDEL

SEQ ID NO: 296 - protein, 40 aa, Artificial Sequence
  Mutated AChE peptide derived from Torpedo californica
  DOMAIN (1)..(40): Mutated AChE peptide
ETIDEAERQWKTEFHRWSCYCMHWKNQFDQYSRHENKDEL

SEQ ID NO: 297 - protein, 32 aa, Artificial Sequence
  Mutated AChE peptide derived from Torpedo californica
  DOMAIN (1)..(32): Mutated AChE peptide
ETIDEAERQWKTEFHRWSSYMMHWKNQFKDEL

SEQ ID NO: 298 - protein, 32 aa, Artificial Sequence
  Mutated AChE peptide derived from Torpedo californica
  DOMAIN (1)..(32): Mutated AChE peptide
ETIDEAERQWKTEFHRWSCYMMHWKNQFKDEL

SEQ ID NO: 299 - protein, 32 aa, Artificial Sequence
  Mutated AChE peptide derived from Torpedo californica
  DOMAIN (1)..(32): Mutated AChE peptide
ETIDEAERQWKTEFHRWSSYCMHWKNQFKDEL

SEQ ID NO: 300 - protein, 32 aa, Artificial Sequence
  Mutated AChE peptide derived from Torpedo californica
  DOMAIN (1)..(32): Mutated AChE peptide
ETIDEAERQWKTEFHRWSCYCMHWKNQFKDEL

SEQ ID NO: 301 - protein, 35 aa, Artificial Sequence
  Mutated AChE peptide derived from Torpedo californica
  DOMAIN (1)..(35): Mutated AChE peptide
ETIDEAERQWKTEFHRWSSYMMHWKNQFDQYKDEL

SEQ ID NO: 302 - protein, 35 aa, Artificial Sequence
  Mutated AChE peptide derived from Torpedo californica
ETIDEAERQWKTEFHRWSCYMMHWKNQFDQYKDEL

SEQ ID NO: 303 - protein, 35 aa, Artificial Sequence
  Mutated AChE peptide derived from Torpedo californica
ETIDEAERQWKTEFHRWSSYCMHWKNQFDQYKDEL

SEQ ID NO: 304 - protein, 35 aa, Artificial Sequence
  Mutated AChE peptide derived from Torpedo californica
ETIDEAERQWKTEFHRWSCYCMHWKNQFDQYKDEL

SEQ ID NO: 305 - protein, 10 aa, Homo sapiens
  DOMAIN (1)..(10): C-Myc tag
EQKLISEEDL

SEQ ID NO: 306 - protein, 319 aa, Escherichia coli; identical to SEQ ID NO: 258
  DOMAIN (1)..(319): Stx1b/VT1b A subunit

SEQ ID NO: 307 - protein, 319 aa, Escherichia coli; identical to SEQ ID NO: 259
  DOMAIN (1)..(319): Stx2e/VT2e A subunit peptide

SEQ ID NO: 308 - protein, 42 aa, Artificial Sequence
  Stx1b-A2-modified peptide linker in which the naturally occurring C at position 10 is replaced by the isosteric S
MASDEFPSMSPADGRVRGITHNKILWDSSTLGAILMRRTISS

SEQ ID NO: 309 - protein, 47 aa, Artificial Sequence
  Stx2a-A2-modified peptide linker in which the naturally occurring C at position 10 is replaced by the isosteric S
AVNEESQPESQITGDRPVIKINNTLWESNTAAAFLNRKSQFLYTTGK

SEQ ID NO: 310 - protein, 46 aa, Artificial Sequence
  Cholera toxin-A2-modified peptide linker
MSNTSDEKTQSLGVKFLDEYQSKVKRQIFSGYQSDIDTHNRIKDEL

SEQ ID NO: 311 - protein, 558 aa, Oryctolagus cuniculus
  DOMAIN (1)..(558): AMF
MAALTRNPQFQKLQQWHREHGSELNLRHLFDTDKERFNHFSLTLNTNHGHILLDYSKNLVTEEVMHMLLDLAKSRGVEAARESMFNGEKINSTEDRAVLHVALRNRSNTPIVVDGKDVMPEVNKVLDKMKAFCQRVRSGDWKGYTGKTITDVINIGIGGSDLGPLMVTEALKPYSSGGPRVWFVSNIDGTHIAKTLACLNPESSLFIIASKTFTTQETITNAKTAKDWFLLSAKDPSTVAKHFVALSTNTAKVKEFGIDPQNMFEFWDWVGGRYSLWSAIGLSIALHVGFDNFEQLLSGAHWMDQHFRTTPLEKNAPVLLAMLGIWYINCFGCETQAVLPYDQYLHRFAAYFQQGDMESNGKYITKSGARVDHQTGPIVWGEPGTNGQHAFYQLIHQGTKMIPCDFLIPVQTQHPIRKGLHHKILLANFLAQTEALMKGKSTEEARKELQAAGKSPEDLMKLLPHKVFEGNRPTNSIVFTKLTPFILGALIAMYEHKIFVQGVVWDINSFDQWGVELGKQLAKKIEPELDGSSPVTSHDSSTNGLINFIKQQREAKIQ